(19) United States
(12) Patent Application Publication
(10) Pub. No.: US 2024/0140957 A1
(43) Pub. Date: May 2, 2024

(54) BRIDGED COMPOUNDS AS KRAS G12D INHIBITOR AND DEGRADER AND THE USE THEREOF

(71) Applicant: BeiGene Switzerland GmbH, Basel (CH)

(72) Inventors: Qi JI, Beijing (CN); Chao YU, Beijing (CN); Ce WANG, Beijing (CN); Hanzi sun, Beijing (CN); Hao YUAN, Beijing (CN); Zhiwei WANG, Beijing (CN)

(21) Appl. No.: 18/260,649

(22) PCT Filed: Jan. 7, 2022
(86) PCT No.: PCT/CN2022/070676

§ 371 Date: Jul. 7, 2023

(30) Foreign Application Priority Data
Jan. 8, 2021 (WO) PCT/CN2021/070898
Feb. 5, 2021 (WO) PCT/CN2021/075582
Apr. 12, 2021 (WO) PCT/CN2021/086638
Jul. 1, 2021 (WO) PCT/CN2021/103945

(51) Int. Cl. C07D 487/08; C07D 239/95; C07D 403/04; C07D 417/14; C07D 471/08; C07D 498/08; C07D 519/00

(52) U.S. Cl. CPC C07D 487/08; C07D 239/95; C07D 403/04; C07D 417/14; C07D 471/08; C07D 498/08; C07D 519/00

(57) ABSTRACT

Provided herein are Bridged Compounds having the following structures:

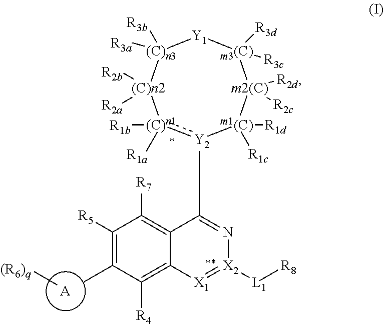

wherein R₁ₐ, R_{1b}, R_{1c}, R_{1d}, R₂ₐ, R_{2b}, R_{2c}, R_{2d}, R₃ₐ, R_{3b}, R_{3c}, R_{3d}, R₄, R₅, R₆, R₇, R₈, m1, m2, m3, n2, n3, n4, q, X₁, X₂, Y₁, Y₂, L₁ and ring A are as defined herein, compositions comprising an effective amount of a Bridged Compound, and methods for treating or preventing various diseases, e.g., pancreatic cancer, or a condition treatable or preventable by inhibition of the function of KRAS protein. In another aspect, a Bridged Compound is useful for treating or preventing a condition treatable or preventable by inhibition of the function of KRAS protein with G12D mutation. In another aspect, a Bridged Compound is useful for treating or preventing a condition treatable or preventable by inhibition of a RAS/MAPK pathway.

### FIELD

Provided herein are certain bridged compounds, compositions comprising an effective amount of such compounds, and methods for treating or preventing various diseases, e.g., pancreatic cancer, or a condition treatable or preventable by inhibition of the function of KRAS protein, comprising administering an effective amount of such compounds to a subject in need thereof.

### BACKGROUND

Ras is a family of proteins which are associated with cell membrane through their C-terminal membrane targeting region and well known as the molecular switch in intracellular signaling network (Cox A D, Der C J. Ras history: The saga continues. Small GTPases. 2010; 1(1):2-27). Ras proteins bind with either GTP or GDP and switch between “on” and “off” states. When Ras proteins bind with GDP, it is in the off (or inactive) state. And when Ras is switched on by certain growth promoting stimuli like growth factors, Ras proteins will be induced to exchange its bound GDP for a GTP and turn into on (or active) state (Malumbres M, Barbacid M. RAS oncogenes: the first 30 years. Nat Rev Cancer. 2003; 3(6):459-465). By switching to active state, Ras protein can interact with different downstream proteins and activate related signaling pathways (Berndt N, Hamilton A D, Sebti S M. Targeting protein prenylation for cancer therapy. Nat Rev Cancer. 2011; 11(11):775-791). Ras superfamily contains different subfamilies including Ras, Ral, Rap, Rheb, Rad, Rit and Miro (Wennerberg K, Rossman K L, Der C J. The Ras superfamily at a glance. J Cell Sci. 2005;118(Pt 5):843-846). HRas, NRas and KRas are the most well studied proteins in Ras family since these proteins are the most common oncogenes in human cancers (O'Bryan J P. Pharmacological targeting of RAS: Recent success with direct inhibitors. Pharmacol Res. 2019; 139:503-511).

KRas is one of the most frequently mutated genes in human cancers. Based on data from Catalogue of Somatic Mutations (COSMIC) database, KRas mutation can be found in about 20% of human cancers, including pancreatic cancer, colorectal cancer, lung cancer, skin cancer etc. (O'Bryan J P. Pharmacological targeting of RAS: Recent success with direct inhibitors. Pharmacol Res. 2019; 139:503-511). And the most common KRas mutations are found at position G12 and G13 by blocking the GTPase activating proteins (GAP) stimulated GTP hydrolysis activity of KRas (Wang W, Fang G, Rudolph J. Ras inhibition via direct Ras binding—is there a path forward?. Bioorg Med Chem Lett. 2012; 22(18):5766-5776). That results in the over activation of KRas protein and ultimately lead to uncontrolled cell proliferation and cancer.

Among different cancers, pancreatic cancer is considered as the most KRas-addicted cancer type. KRas mutation is found in 94.1% of pancreatic ductal adenocarcinoma (PDAC). G12D (41%) and G12V (34%) mutations of KRas are the two most predominant mutations in all the KRas mutated PDAC (Waters A M, Der C J. KRAS: The Critical Driver and Therapeutic Target for Pancreatic Cancer. Cold Spring Harb Perspect Med. 2018;8(9):a031435). In vivo data generated by mouse models proves that the progression and maintenance of pancreatic cancer are highly rely on the constitutive activation of KRas downstream signaling (Siveke J T, Schmid R M. Chromosomal instability in mouse metastatic pancreatic cancer—it's Kras and Tp53 after all. Cancer Cell. 2005; 7(5):405-407). Which indicates that mutated KRas protein is a highly attractive drug target for pancreatic cancer and also other cancers with KRas mutation. Since WT KRas protein also plays a critical role in the function of normal tissue and WT KRas function is demonstrated to be essential for adult hematopoiesis (Malumbres M, Barbacid M. RAS oncogenes: the first 30 years. Nat Rev Cancer. 2003; 3(6):459-465). It is highly deserved that a potential drug molecule can selectively inhibit mutated Kras protein in cancer cells and spare its WT companion in normal cells. Because Kras protein is generally considered as a non-druggable target, there is no therapeutics which can selectively target Kras protein with G12D mutation in clinic.

There are two strategies a drug molecule can adopt to selectively eliminate the over activated Kras signaling which induced by Kras mutations. One way is to directly bind with the mutated Kras protein, either by stabilizing its GDP bound form (the inactive form) or by blocking the interaction between GTP bound form and its downstream target protein. Another strategy is to hijack the protein degradation mechanism in cell and leverage E3 ligases' (like VHL, CRBN or IAPs) substrate specificity through a bi-functional molecule called Proteolysis targeting chimera (PROTAC) (Winter G E, Buckley D L, Paulk J, Roberts J M, Souza A, Dhe-Paganon S, Bradner J E. DRUG DEVELOPMENT. Phthalimide conjugation as a strategy for in vivo target protein degradation. Science. 2015 Jun. 19; 348(6241):1376-81). Which can bind with both mutated Kras protein and E3 ligase, create interactions between those two proteins and induced Kras degradation.

Thus, KRas G12D mutation is a highly attractive target for pancreatic cancer and other cancers with this mutation. As such, small-molecule therapeutic agents that are capable to selectively bind with Kras G12D and inhibit its function would be very useful. And Kras G12D targeting bi-functional PROTAC is also an attractive strategy to target cancers with this mutation.

Citation or identification of any reference in this section of this application is not to be construed as an admission that the reference is prior art to the present application.

### SUMMARY

Provided herein are compounds having the following formula (I):

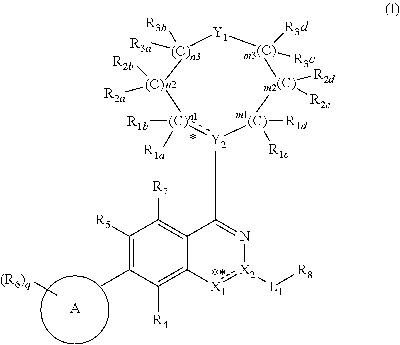

and pharmaceutically acceptable salts, tautomers, isotopologues, stereoisomers and prodrugs thereof, wherein R₁ₐ, R_{1b}, R_{1c}, R₁ₐ, R₂ₐ, R_{2b}, R_{2c}, R_{2d}, R₃ₐ, R_{3b}, R_{3c}, R_{3d}, R₄, R₅, R₆, R₇, R₈, m1, m2, m3, n2, n3, n4, q, X₁, X₂, Y₁, Y₂, L₁ and ring A are as defined herein.

Provided herein are compounds having the following formula (II):

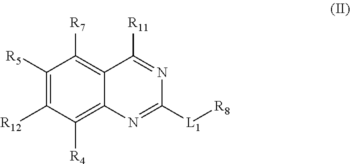

and pharmaceutically acceptable salts, tautomers, isotopologues, stereoisomers and prodrugs thereof, wherein R₄, R₅, R₇, R₈, R₁₁, R₁₂ and L₁ are as defined herein.

A compound of formula (I) or formula (II) or a pharmaceutically acceptable salt, tautomer, isotopologue, stereoisomer or prodrug thereof (each being referred to herein as “Bridged Compounds”) is useful for treating or preventing various diseases, e.g., pancreatic cancer, as described herein. In another aspect, a Bridged Compound is useful for treating or preventing pancreatic cancer as described herein. In another aspect, a Bridged Compound is useful for treating or preventing a condition treatable or preventable by inhibition of the function of KRAS protein, as described herein. In another aspect, a Bridged Compound is useful for treating or preventing a condition treatable or preventable by inhibition of the function of KRAS protein with G12D mutation, as described herein. In another aspect, a Bridged Compound is useful for treating or preventing a condition treatable or preventable by inhibition of a RAS/MAPK pathway, as described herein.

In one aspect, provided herein are Bridged Compounds as described in the instant disclosure, such as, for example, in Table 1, Table 2, and Table 6.

In one aspect, provided herein are pharmaceutical compositions comprising an effective amount of a Bridged Compound as described herein, and a pharmaceutically acceptable carrier, excipient or vehicle. In some embodiments the pharmaceutical composition is suitable for oral, parenteral, mucosal, transdermal or topical administration.

In one aspect, provided herein are methods for treating or preventing pancreatic cancer, comprising administering to a subject in need thereof an effective amount of a Bridged Compound disclosed herein; or the use of the Bridged Compound disclosed herein in the manufacture of a medicament for treating or preventing pancreatic cancer; or the Bridged Compound disclosed herein for use in treating or preventing pancreatic cancer. In another aspect, provided herein are methods for treating or preventing a condition treatable or preventable by inhibition of a RAS/MAPK pathway, comprising administering to a subject in need thereof an effective amount of a Bridged Compound as described herein. In one aspect, provided herein are methods for inhibiting the function of a protein, for example KRAS protein, in a cell expressing said protein, comprising contacting said cell with an effective amount of a Bridged Compound as described herein. In one aspect, provided herein are methods for inhibiting the function of a protein, for example KRAS protein with G12D mutation, in a cell expressing said protein, comprising contacting said cell with an effective amount of a Bridged Compound as described herein.

In another aspect provided herein are methods for preparing Bridged Compounds as described herein.

### DETAILED DESCRIPTION

### Definitions

As used herein, “KRAS gene” refers to a gene selected from the group consisting of: DIRAS1; DIRAS2; DIRAS3; ERAS; GEM; HRAS; KRAS; MRAS; NKIRAS1; NKIRAS2; NRAS; RALA; RALB; RAP1A; RAP1B; RAP2A; RAP2B; RAP2C; RASD1; RASD2; RASL10A; RASL10B; RASL11A; RASL11B; RASL12; REM1; REM2; RERG; RERGL; RRAD; RRAS; RRAS2, and mutants thereof.

As used herein, “KRAS protein” refers to a protein or an isoform thereof expressed by a KRAS gene (Scolnick E M, Papageoege A G, Shih T Y (1979), “Guanine nucleotide-binding activity for src protein of rat-derived murine sarcoma viruses,” Proc Natl Acad Sci USA. 76 (5): 5355-5559; Kranenburg O (November 2005) “The KRAS oncogene: past, present, and future,” Biochimica et Biophysica Acta (BBA)—Reviews on Cancer, 1756 (2): 81-2).

As used herein, “G12D mutation” refers to that amino acid position 12 of the KRAS protein is occupied by aspartic acid.

As used herein, “KRAS G12D” refers to KRAS protein with G12D mutation.

As used herein, “bridged bicyclic ring” refers to a cyclic structure comprising two rings sharing three or more atoms, separating the two bridgehead atoms by a bridge containing at least one atom. In some embodiments, the bridged bicyclic ring may optionally comprise one or two double bonds in the ring structure. In some embodiments, the bridged bicyclic ring may independently comprise one or more, preferably one to two, heteroatoms independently selected from nitrogen, oxygen, and sulfur. Examples of bridged bicyclic rings include, but are not limited to, 1-bicyclo[1.1.1]pentyl, bicyclo[2.1.1]hexyl, bicyclo[2.2.1]heptyl, bicyclo[2.2.2]octyl, bicyclo[3.3.1]nonyl, adamantyl, 3,8-diazabicyclo[3.2.1]octane, 2,5-diazabicyclo[2.2.1]heptane, 2,5-diazabicyclo[2.2.2]octane, 3,6-diazabicyclo[3.1.1]heptane, 3-oxa-7,9-diazabicyclo[3.3.1]nonane, 7-azabicyclo[2.2.1]heptane, 8-azabicyclo[3.2.1]oct-2-ene-3-yl, and the like, which may independently comprise one or more, preferably one to two, heteroatoms independently selected from nitrogen, oxygen, and sulfur.

As used herein, “spirocyclic ring” refers to two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocyclic rings may be substituted or unsubstituted cycloalkyl, substituted or unsubstituted cycloalkylene, substituted or unsubstituted heterocycloalkyl or substituted or unsubstituted heterocycloalkelyene and individual rings within a spirocyclic ring group may be any of the immediately previous list, including having all rings of one type (e.g. all rings being substituted heterocycloalkelyene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₇CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

A “cycloalkyl” group is a saturated, partially saturated, or partially unsaturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

The term “bridged bicyclic ring” herein refers to a cyclic structure comprising two rings sharing three or more atoms, separating the two bridgehead atoms by a bridge containing at least one atom. In some embodiments, the bridged bicyclic ring may optionally comprise one or two double bonds in the ring structure. In some embodiments, the bridged bicyclic ring may independently comprise one or more, preferably one to two, heteroatoms independently selected from nitrogen, oxygen, and sulfur.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aryl ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1H-benzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4-dihydroisoquinolin-1(2H)-onyl), tetrahydroquinolinyl, quinoxalinyl, and quinazolinyl groups.

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocycloalkyl group can be substituted or unsubstituted. Heterocyclyl groups encompass unsaturated, partially saturated and saturated ring systems, such as, for example, imidazolyl, imidazolinyl and imidazolidinyl (e.g., imidazolidin-4-one or imidazolidin-2,4-dionyl) groups. The phrase heterocyclyl includes fused ring species, including those comprising fused aromatic and non-aromatic groups, such as, for example, 1- and 2-aminotetraline, benzotriazolyl (e.g., 1H-benzo[d][1,2,3]triazolyl), benzimidazolyl (e.g., 1H-benzo[d]imidazolyl), 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, azepanyl, oxetanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, benzisoxazolyl (e.g., benzo[d]isoxazolyl), thiazolyl, thiazolinyl, isothiazolyl, thiadiazolyl, oxadiazolyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dioxyl, dithianyl, pyranyl, pyridyl, pyrimidyl, pyridazinyl, pyrazinyl, triazinyl, dihydropyridyl, dihydrodithiinyl, dihydrodithionyl, 1,4-dioxaspiro[4.5]decanyl, homopiperazinyl, quinuclidyl, indolyl (e.g., indolyl-2-onyl or isoindolin-1-onyl), indolinyl, isoindolyl, isoindolinyl, azaindolyl (pyrrolopyridyl or 1H-pyrrolo[2,3-b]pyridyl), indazolyl, indolizinyl, benzotriazolyl (e.g. 1H-benzo[d][1,2,3]triazolyl), benzimidazolyl (e.g., 1H-benzo[d]imidazolyl or 1H-benzo[d]imidazol-2(3H)-onyl), benzofuranyl, benzothiophenyl, benzothiazolyl, benzoxadiazolyl, benzoxazinyl, benzodithiinyl, benzoxathiinyl, benzothiazinyl, benzoxazolyl (i.e., benzo[d]oxazolyl), benzothiazolyl, benzothiadiazolyl, benzo[1,3]dioxolyl, pyrazolopyridyl (for example, 1H-pyrazolo[3,4-b]pyridyl, 1H-pyrazolo[4,3-b]pyridyl), imidazopyridyl (e.g., azabenzimidazolyl or 1H-imidazo[4,5-b]pyridyl), triazolopyridyl, isoxazolopyridyl, purinyl, xanthinyl, adeninyl, guaninyl, quinolinyl, isoquinolinyl (e.g., 3,4-dihydroisoquinolin-1(2H)-onyl), quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, tetrahydropyrimidin-2(1H)-one and tetrahydroquinolinyl groups. Representative non-aromatic heterocyclyl groups do not include fused ring species that comprise a fused aromatic group. Examples of non-aromatic heterocyclyl groups include aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, ortetrahydropyrimidin-2(1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “cycloalkylalkyl” group is a radical of the formula: -alkyl-cycloalkyl, wherein alkyl and cycloalkyl are as defined above. Substituted cycloalkylalkyl groups may be substituted at the alkyl, the cycloalkyl, or both the alkyl and the cycloalkyl portions of the group. Representative cycloalkylalkyl groups include but are not limited to methylcyclopropyl, methylcyclobutyl, methylcyclopentyl, methylcyclohexyl, ethylcyclopropyl, ethylcyclobutyl, ethylcyclopentyl, ethylcyclohexyl, propylcyclopentyl, propylcyclohexyl and the like.

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethyl-indanyl.

An “heterocyclylalkyl” group is a radical of the formula: -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocylylalkyl groups include but are not limited to 4-ethyl-morpholinyl, 4-propylmorpholinyl, furan-2-yl methyl, furan-3-yl methyl, pyridin-3-yl methyl, tetrahydrofuran-2-yl ethyl, and indol-2-yl propyl.

An “heteroaralkyl” group is a radical of the formula: -alkyl-heteroaryl, wherein alkyl and heteroaryl are defined above. Substituted heteroaralkyl groups may be substituted at the alkyl, the heteroaryl, or both the alkyl and the heteroaryl portions of the group.

An “-alkyl-bridged bicyclic ring” group is a radical of the formula: -alkyl-bridged bicyclic ring, wherein alkyl and bridged bicyclic ring are defined above. Substituted -alkyl-bridged bicyclic ring groups may be substituted at the alkyl, the bridged bicyclic ring, or both the alkyl and the bridged bicyclic ring portions of the group.

An “-alkyl-spirocyclic ring” group is a radical of the formula: -alkyl-spirocyclic ring, wherein alkyl and spirocyclic ring are defined above. Substituted -alkyl-spirocyclic ring groups may be substituted at the alkyl, the spirocyclic ring, or both the alkyl and the spirocyclic ring portions of the group.

A “halogen” is fluorine, chlorine, bromine or iodine.

A “hydroxyalkyl” group is an alkyl group as described above substituted with one or more hydroxy groups.

An “alkoxy” group is —O-(alkyl), wherein alkyl is defined above.

An “alkoxyalkyl” group is -(alkyl)-O-(alkyl), wherein alkyl is defined above.

An “amino” group is a radical of the formula: —NH₂.

An “alkylamino” group is a radical of the formula: —NH-alkyl or —N(alkyl)₂, wherein each alkyl is independently as defined above.

A “carboxy” group is a radical of the formula: —C(O)OH.

An “aminocarbonyl” group is a radical of the formula: —C(O)N(R^{#})₂, —C(O)NH(R^{#}) or —C(O)NH₂, wherein each R^{#} is independently a substituted or unsubstituted alkyl, cycloalkyl, aryl, aralkyl, heterocyclyl or heterocyclyl group as defined herein.

An “acylamino” group is a radical of the formula: —NHC(O)(R^{#}) or —N(alkyl)C(O)(R^{#}), wherein each alkyl and R^{#} are independently as defined above.

A “sulfonylamino” group is a radical of the formula: —NHSO₂(R^{#}) or —N(alkyl)SO₂(R^{#}), wherein each alkyl and R^{#} are defined above.

A “urea” group is a radical of the formula: —N(alkyl)C(O)N(R^{#})₂, —N(alkyl)C(O)NH(R^{#}), —N(alkyl)C(O)NH₂, —NHC(O)N(R^{#})₂, —NHC(O)NH(R^{#}), or —NH(CO)NHR #, wherein each alkyl and R^{#} are independently as defined above.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (—O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

As used herein, the term “Bridged Compound” refers to the compounds of Formulas (I)-(XV) as well as to further embodiments provided herein. In one embodiment, a “Bridged Compound” is a compound set forth in Table 1, Table 2, and Table 6. The term “Bridged Compound” includes pharmaceutically acceptable salts, tautomers, isotopologues, stereoisomers, and prodrugs of the compounds provided herein.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable salts of the Bridged Compounds of formulas (I) and (II) include, but are not limited to, those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

As used herein and unless otherwise indicated, the term “prodrug” means a Bridged Compound that can hydrolyze, oxidize, or otherwise react under biological conditions (in vitro or in vivo) to provide an active compound, particularly a Bridged Compound or a compound of formulas (I) and (II). Examples of prodrugs include, but are not limited to, derivatives and metabolites of a Bridged Compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a Bridged Compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The Bridged Compounds can have chiral centers and can occur as racemates, individual enantiomers or diastereomers, and mixtures thereof. All such isomeric forms are included within the embodiments disclosed herein, including mixtures thereof.

The use of stereomerically pure forms of such Bridged Compounds, as well as the use of mixtures of those forms, are encompassed by the embodiments disclosed herein. For example, mixtures comprising equal or unequal amounts of the enantiomers of a particular Bridged Compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., i Tables of Resolving Agents and Optical Resolutions p. 268 (E. L. Eliel, Ed., Univ. of Notre Dame Press, Notre Dame, IN, 1972).

It should also be noted the Bridged Compounds can include E and Z isomers, or a mixture thereof, and cis and trans isomers or a mixture thereof. In certain embodiments, the Bridged Compounds are isolated as either the E or Z isomer. In other embodiments, the Bridged Compounds are a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms which are referred to as tautomers of each other:

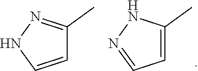

As readily understood by one skilled in the art, a wide variety of functional groups and other structures may exhibit tautomerism and all tautomers of compounds of formula (I) are within the scope of the present invention.

It should also be noted the Bridged Compounds can contain unnatural proportions of atomic isotopes at one or more of the atoms. For example, the Bridged Compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the Bridged Compounds as described herein whether radioactive or not, are intended to be encompassed within the scope of the embodiments provided herein. In some embodiments, there are provided isotopologues of the Bridged Compounds, for example, the isotopologues are deuterium, carbon-13, or nitrogen-15 enriched Bridged Compounds.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In one embodiment, the disorder is pancreatic cancer. In some embodiments, “treating” means an alleviation, in whole or in part, of pancreatic cancer. In one embodiment, the symptom is pancreatic cancer. In another embodiment, “treating” means and alleviation, in whole or in part, of a disorder, disease or condition, or symptoms associated with a condition, treatable or preventable by inhibition of a RAS/MAPK pathway.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the disorder is pancreatic cancer, as described herein, or symptoms thereof. In another, the disorder is a condition, treatable or preventable by inhibition of a RAS/MAPK pathway.

The term “effective amount” in connection with a Bridged Compound means an amount capable of treating or preventing a disorder, disease or condition, or symptoms thereof, disclosed herein.

The term “subject” includes an animal, including, but not limited to, an animal such a cow, monkey, horse, sheep, pig, chicken, turkey, quail, cat, dog, mouse, rat, rabbit or guinea pig, in one embodiment a mammal, in another embodiment a human. In one embodiment, a subject is a human having or at risk for having pancreatic cancer, or a condition, treatable or preventable by inhibition of a RAS/MAPK pathway, or a symptom thereof.

### Bridged Compounds

Aspect 1: Provided herein are bridged compounds having the following formula (I):

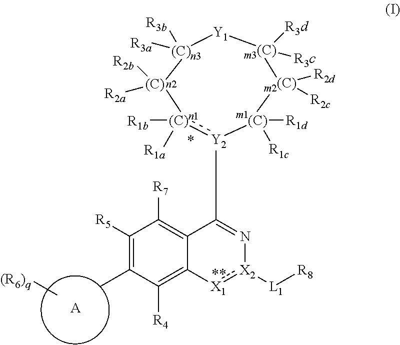

and pharmaceutically acceptable salts, tautomers, isotopologues, stereoisomers and prodrugs thereof, wherein:
ring A is an aryl group or a 5- to 7-membered monocyclic heteroaryl or 8- to 12-membered bicyclic heteroaryl group;
[symbol] and [symbol] are each independently a single bond or double bond;
Y₁ is —NH— or —C(R_{Y1a})(NHR_{Y1b});
Y₂ is C and R_{1b} is absent in the case that [symbol] is a double bond; or Y₂ is N or CR_{Y2} in the case that [symbol] is a single bond;
X₁ is N, CH or CO; X₂ is C or N;
provided that when [symbol] is a double bond, X₁ is N or CH, X₂ is C; or
   when [symbol] is a single bond, X₁ is CO, X₂ is N;
n1, n2, n3, m1, m2, and m3 are each independently 0 or 1, provided that at least one of n1, n2 and n3 is 1; and at least one of m1, m2 and m3 is 1;
q is 0, 1, 2, 3, 4, 5, or 6;
R₁ₐ, R_{1b}, R₂ₐ, R_{2b}, R₃ₐ, R_{3b}, R_{1c}, R₁ₐ, R_{2c}, R_{2d}, R_{3c}, R_{3d}, R_{Y1a}, R_{Y1b} and R_{Y2}, if present, are each independently hydrogen, halogen, —C₁₋₈-alkyl, C₃-C₈cycloalkyl, 3- to 8-membered heterocyclyl, C₆-C₁₂aryl, 5- to 12-membered heteroaryl, —CN, —OR₁ₑ, or —NR₁ₑR_{1f}; each of —C₁₋₈alkyl, C₃-C₈cycloalkyl, 3- to 8-membered heterocyclyl, C₆-C₁₂aryl or 5- to 12-membered heteroaryl is optionally substituted with at least one substituent R_{1g}; and
at least one pair of (R₁ₐ and R_{1c}), (R₁ₐ and R_{2c}), (R₁ₐ and R_{3c}), (R₂ₐ and R_{1c}), (R₂ₐ and R_{2c}), (R₂ₐ and R_{3c}), (R₃ₐ and R_{1c}), (R₃ₐ and R_{2c}), (R₃ₐ and R_{3c}), (R_{Y1a} and R_{Y2}), (R_{Y1a} and R₁ₐ), (R_{Y1a} and R₂ₐ), (R_{Y1a} and R_{1c}), (R_{Y1a} and R_{2c}), (R_{Y1b} and R₁ₐ), (R_{Y1b} and R₂ₐ), (R_{Y1b} and R_{1c}), (R_{Y1b} and R_{2c}), and (R_{Y1b} and R_{Y2}) form a bridge containing one, two, three, or four —CH₂— moieties in addition to the two bridgeheads, wherein one of the —CH₂— moiety is optionally replaced with —O—, —S— or —NH— and wherein said bridge is optionally substituted with at least one substituent R_{1g};
optionally, (R_{Y1a} and R_{Y1b}), (R_{Y1a} and R₃ₐ), (R_{Y1a} and R_{3c}), (R_{Y1b} and R₃ₐ), or (R_{Y1b} and R_{3c}) form 3- to 12-membered ring, the said ring comprises 0-3 heteroatoms selected from nitrogen, sulfur and oxygen and the said bridge is optionally substituted with at least one substituent R_{1g};
R₁ₑ and R_{1f} are each independently selected from hydrogen, —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl;
R_{1g}, at each occurrence, is independently halogen, —C₁₋₈-alkyl, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl, —C₁₋₈haloalkyl, C₁₋₈alkoxy-C₁₋₈alkyl-, —CN, —OH, —NH₂, —C₁₋₈alkoxyl;
R₆ is hydrogen, halogen, —C₁₋₈-alkyl, —C₂₋₈alkenyl, —C₂₋₈alkynyl, C₃-C₈cycloalkyl, 3- to 8-membered heterocyclyl, C₆-C₁₂aryl, 5- to 12-membered heteroaryl, —CN, oxo, —OR₆ₐ, —SR₆ₐ, —SO₂R₆ₐ, —SO₂NR₆ₐR_{6b}, —COR₆ₐ, —CO₂R₆ₐ, —CONR₆ₐR_{6b}, —NR₆ₐR_{6b}, —NR₆ₐCOR_{6b}, —NR₆ₐCO₂R_{6b}, or —NR₆ₐSO₂R_{6b}; each of said —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl is optionally substituted with at least one substituent R_{6c}; or
two R₆ together with the atoms to which they are attached, form a 5, 6, 7 or 8-membered unsaturated (preferred aromatic) or saturated ring, said ring comprising 0-3 heteroatoms independently selected from nitrogen, oxygen or sulfur; said ring is optionally substituted with at least one substituent R_{6d};
R₆ₐ and R_{6b} are each independently selected from hydrogen, —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl, wherein each of said —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl is optionally substituted with at least one substituent R_{6d};
R_{6c}, at each occurrence, is independently halogen, —C₁₋₈-alkyl, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R_{6d} is hydrogen, halogen, —C₁₋₈-alkyl, —C₂₋₈alkenyl, —C₂₋₈alkynyl, C₃-C₈cycloalkyl, 3- to 8-membered heterocyclyl, C₆-C₁₂aryl, 5- to 12-membered heteroaryl, —CN, oxo, —OR₆ₑ, —SO₂R₆ₑ, —SO₂NR₆ₑR_{6f}, —COR₆ₑ, —CO₂R₆ₑ, —CONR₆ₑR_{6f}, —NR₆ₑR_{6f}, —NR₆ₑCOR_{6f}, —NR₆ₑCO₂R_{6f}, or —NR₆ₑSO₂R_{6f}; each of —C₁₋₈alkyl, —C₂-8alkenyl, —C₂₋₈alkynyl, C₃-C₈cycloalkyl, 3- to 8-membered heterocyclyl, C₆-C₁₂aryl or 5- to 12-membered heteroaryl is optionally substituted with at least one substituent R_{6g};
R₆ₑ and R_{6f} are each independently selected from hydrogen, —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl, each of said —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl is optionally substituted with at least one substituent R_{6g};
R_{6g}, at each occurrence, is independently halogen, —C₁₋₈-alkyl, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R₄, R₅, and R₇ are each independently hydrogen, halogen, —C₁₋₈-alkyl, C₃-C₈cycloalkyl, —C₂₋₈alkenyl, —C₂₋₈alkynyl, 3- to 8-membered heterocyclyl, C₆-C₁₂aryl, 5- to 12-membered heteroaryl, —CN, oxo, —OR₄ₐ, —SR₄ₐ, —SO₂R₄ₐ, —SO₂NR₄ₐR_{4b}, —COR₄ₐ, —CO₂R₄ₐ, —CONR₄ₐR_{4b}, —NR₄ₐR_{4b}, —NR₄ₐCOR_{4b}, —NR₄ₐCO₂R_{4b}, or —NR₄ₐSO₂R_{4b}; wherein each of —C₁₋₈alkyl, C₃-C₈cycloalkyl, —C₂₋₈alkenyl, —C₂₋₈alkynyl, 3- to 8-membered heterocyclyl, C₆-C₁₂aryl or 5- to 12-membered heteroaryl is optionally substituted with at least one substituent R_{4c},
R₄ₐ and R_{4b} are each independently selected from hydrogen, —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl; wherein each of —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl is optionally substituted with at least one substituent R_{4d};
R_{4c} and R_{4d}, at each occurrence, are each independently halogen, —C₁₋₈-alkyl, —C₂₋₈alkenyl, —C₂-8alkynyl, —C₃-C₈cycloalkyl, 3- to 8-membered heterocyclyl, —C₆-C₁₂aryl, or 5- to 12-membered heteroaryl;
L₁ is selected from a single bond, —O—, —S—, —NR^{L1a}—, —C(O)—, —C₁₋₈alkylene-, *^{L1}—O—C₁₋₈alkylene-**^{L1}, *^{L1}—C₁₋₈alkylene-O—**^{L1}, *^{L1}—S—C₁₋₈alkylene-**^{L1}, *^{L1}—C₁₋₈alkylene-S—**^{L1}, —C₃-C₈cycloalkylene-, *^{L1}—O—C₃-C₈cycloalkylene-**^{L1}, *^{L1}—C₃-C₈cycloalkylene-O—**^{L1}, *^{L1}—S—C₃-C₈cycloalkylene-**^{L1}, *^{L1}—C₃-C₈cycloalkylene-S—**^{L1}, *^{L1}—O—C₁₋₈alkylene-NR^{L1a}—**^{L1}, *^{L1}—NR^{L1a}—C₁₋₈alkylene-O—**^{L1}, *^{L1}—S—C₁₋₈alkylene-NR^{L1a}—**^{L1}, *^{L1}—NR^{L1a}—C₁₋₈alkylene-S—**^{L1}, *^{L1}—O—C₁₋₈alkylene-C(O)—**^{L1}, *^{L1}—C(O)—C₁₋₈alkylene-O—**^{L1}, *^{L1}—S—C₁₋₈alkylene-C(O)—**^{L1}, *^{L1}—C(O)—C₁₋₈alkylene-S—**^{L1}, *^{L1}—C(O)—C₁₋₈alkylene-**^{L1}, *^{L1}—C₁₋₈alkylene-C(O)—**^{L1}, *^{L1}—NR^{L1a}—C₁₋₈alkylene-**^{L1}, *^{L1}—C₁₋₈alkylene-NR^{L1a}—**^{L1}, *^{L1}—C(O)NR^{L1a}—C₁₋₈alkylene-**^{L1}, *^{L1}—C₁₋₈alkylene-C(O)NR^{L1a}—**^{L1}, *^{L1}—NR^{L1a}C(O)—C₁₋₈alkylene-**^{L1}, *^{L1}—C₁₋₈alkylene-NR^{L1a}C(O)—**^{L1}, *^{L1}—C(O)O—C₁₋₈alkylene-**^{L1}, *^{L1}—C₁₋₈alkylene-C(O)O—**^{L1}, *^{L1}—OC(O)—C₁₋₈alkylene-**^{L1}, *^{L1}—C₁₋₈alkylene-OC(O)—**^{L1},

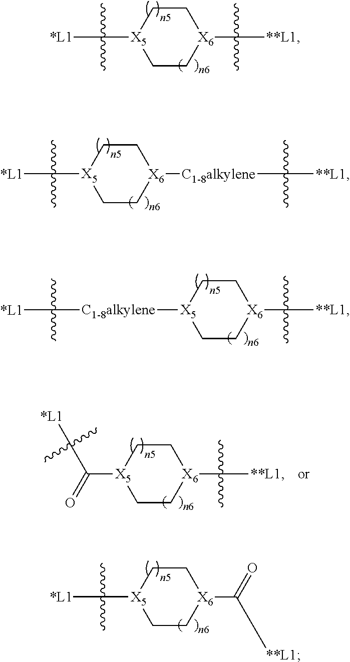

each of said single bond, —C₁₋₈alkylene-, *^{L1}—O—C₁₋₈alkylene-**^{L1}, *^{L1}—C₁₋₈alkylene-O—**^{L1}, *^{L1}—S—C₁₋₈alkylene-**^{L1}, *^{L1}—C₁₋₈alkylene-S—**^{L1}, —C₃-C₈cycloalkylene-, *^{L1}—O—C₃-C₈cycloalkylene-**^{L1}, *^{L1}—C₃-C₈cycloalkylene-O—**^{L1}, *^{L1}—S—C₃-C₈cycloalkylene-**^{L1}, *^{L1}—C₃-C₈cycloalkylene-S—**^{L1}, *^{L1}—O—C₁₋₈alkylene-NR^{L1a}—**^{L1}, *^{L1}—NR^{L1a}—C₁₋₈alkylene-O—**^{L1}, *^{L1}—S—C₁₋₈alkylene-NR^{L1a}—**^{L1}, *^{L1}—NR^{L1a}—C₁₋₈alkylene-S—**^{L1}, *^{L1}—O—C₁₋₈alkylene-C(O)—**^{L1}, *^{L1}—C(O)—C₁₋₈alkylene-O—**^{L1}, *^{L1}—S—C₁₋₈alkylene-C(O)—**^{L1}, *^{L1}—C(O)—C₁₋₈alkylene-S—**^{L1}, *^{L1}—C(O)—C₁₋₈alkylene-**^{L1}, *^{L1}—C₁₋₈alkylene-C(O)—**^{L1}, *^{L1}—NR^{L1a}—C₁₋₈alkylene-**^{L1}, *^{L1}—C₁₋₈alkylene-NR^{L1a}—**^{L1}, *^{L1}—C(O)NR^{L1a}—C₁₋₈alkylene-**^{L1}, *^{L1}—C₁₋₈alkylene-C(O)NR^{L1a}—**^{L1}, *^{L1}—NR^{L1a}C(O)—C₁₋₈alkylene-**^{L1}, *^{L1}—C₁₋₈alkylene-NR^{L1a}C(O)—**^{L1}, *^{L1}—C(O)O—C₁₋₈alkylene-**^{L1}, *^{L1}—C₁₋₈alkylene-C(O)O—**^{L1}, *^{L1}—OC(O)—C₁₋₈alkylene-**^{L1}, *^{L1}—C₁₋₈alkylene-OC(O)—**^{L1},

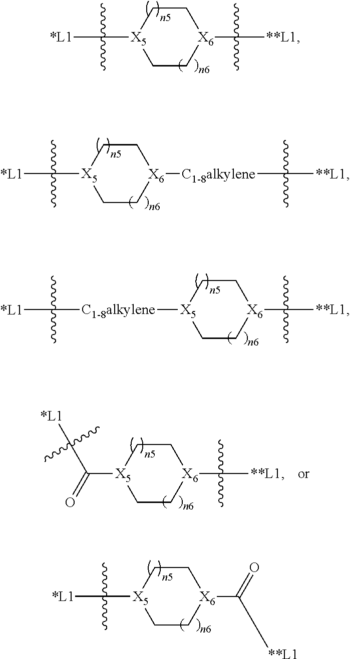

are optionally substituted with at least one R^{L1b};
wherein **^{L1} refers to the position attached to the

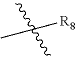

moiety, and *^{L1} refers to the position attached to the other side;
R^{L1a} is selected from hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl, each of said —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl is optionally substituted with at least one substituent R^{L1c};
each of said R^{L1b} and R^{L1c} are independently halogen, hydroxy, —C₁₋₈alkyl, —C₁₋₈alkoxy, —C₂₋₈alkenyl, —C₂₋₈alkynyl, C₃-C₈cycloalkyl, 3- to 8-membered heterocyclyl, C₆-C₁₂aryl or 5- to 12-membered heteroaryl; or
two R^{L1b} or two R^{L1c} together with the atoms to which they are attached, form a 3- to 6-membered unsaturated or saturated ring, said ring comprising 0-3 heteroatoms independently selected from nitrogen, oxygen or sulfur; said ring is optionally substituted with at least one substituent halogen, hydroxy, —C₁₋₈alkyl, —C₁₋₈alkoxy, —C₂₋₈alkenyl, —C₂₋₈alkynyl, C₃-C₈cycloalkyl, 3- to 8-membered heterocyclyl, C₆-C₁₂aryl or 5- to 12-membered heteroaryl;
each of X₅ and X₆ are selected from CH or N;
n5 and n6 are each independently 0, 1 or 2;
R₈ is hydrogen, halogen, —C₁₋₈alkyl, C₃-C₈cycloalkyl, 3- to 12-membered heterocyclyl, C₆-C₁₂aryl, 5- to 12-membered heteroaryl, oxo, —CN, —OR₃ₐ, —COR₈ₐ, —CO₂R₈ₐ, —CONR₈ₐR_{8b}, —NR₈ₐR_{8b}, —NR₈ₐCOR_{8b} or —NR₈ₐCO₂R_{8b}, wherein each of —C₁₋₈alkyl, C₃-C₈cycloalkyl, 3- to 12-membered heterocyclyl, C₆-C₁₂aryl or 5- to 12-membered heteroaryl is optionally substituted with at least one substituent Rse;
R₈ₐ and R_{8b} are each independently selected from hydrogen, —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl, 5- to 12-membered heteroaryl or oxo; wherein each of said —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl, 5- to 12-membered heteroaryl is optionally substituted with at least one substituent R_{8d}; or
R₈ₐ and R_{8b} together with the carbon atoms to which they are attached, form a 3- to 8-membered unsaturated or saturated ring, said ring comprising 0-3 heteroatoms independently selected from nitrogen, oxygen or sulfur; said ring is optionally substituted with at least one substituent R_{8c};
R_{8c}, at each occurrence, is independently halogen, —C₁₋₈-alkyl, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl, oxo, —CN, —OR₈ₑ, —COR₈ₑ, —CO₂R₈ₑ, —CONR₈ₑR_{8f}, —NR₈ₑR_{8f}, —NR₈ₑCOR_{8f} or —NR₈ₑCO₂R_{8f}, wherein each of said —C₁₋₈alkyl, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl is optionally substituted with at least one substituent R_{8d};
two R_{8c} together with the carbon atoms to which they are attached, form a 3- to 8-membered unsaturated or saturated ring, said ring comprising 0-3 heteroatoms independently selected from nitrogen, oxygen or sulfur; said ring is optionally substituted with at least one substituent R_{8d}; or two R_{8c} together with the atoms to which they are attached form a bridge containing one, two, three, or four —CH2- moieties in addition to the two bridgeheads, wherein one of the —CH₂— moiety is optionally replaced with —O—, —S— or —NH—;
R_{8d} is hydrogen, halogen, —C₁₋₈alkyl, C₃-C₈cycloalkyl, 3- to 8-membered heterocyclyl, C₆-C₁₂aryl, 5- to 12-membered heteroaryl, oxo, —CN, —OR_{8g}, —COR_{8g}, —CO₂R_{8g}, —CONR_{8g}R₈ₕ, —NR_{8g}R₈ₕ, —NR_{8g}COR₈ₕ or —NR_{8g}CO₂R₈ₕ, wherein each of —C₁₋₈alkyl, C₃-C₈cycloalkyl, 3- to 8-membered heterocyclyl, C₆-C₁₂aryl or 5- to 12-membered heteroaryl is optionally substituted with at least one substituent R₈ᵢ;
R₈ₑ, R_{8f}, R_{8g}, R₈ₕ and R₈ᵢ are each independently selected from hydrogen, —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl.

Aspect 2: In some embodiments, wherein Y₁ is selected from —NH— or —C(R_{Y1a})(NH₂)—.

Aspect 3: In some embodiments, wherein R₁ₐ, R_{1b}, R₂ₐ, R_{2b}, R₃ₐ, R_{3b}, R_{1c}, R₁ₐ, R_{2c}, R_{2d}, R_{3c}, R_{3d}, R_{Y1a}, R_{Y1b} and R_{Y2}, if present, are each independently hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —OR₁ₑ or —NR₁ₑR_{1f}; each of methyl, ethyl, propyl, butyl, pentyl, hexyl, heptyl, octyl, cyclopropyl, cyclobutyl, cyclopentyl, cyclohexyl, cycloheptyl or cyclooctyl is optionally substituted with at least one substituent R_{1g}, one pair of (R₁ₐ and R_{1c}), (R₁ₐ and R_{2c}), (R₁ₐ and R_{3c}), (R₂ₐ and R_{1c}), (R₂ₐ and R_{2c}), (R₂ₐ and R_{3c}), (R₃ₐ and R_{1c}), (R₃ₐ and R_{2c}), (R₃ₐ and R_{3c}), (R_{Y1a} and R_{Y2}), and (R_{Y1b} and R_{Y2}) form a bridge containing one, two, three, or four —CH₂— moieties in addition to the two bridgeheads, wherein one of the —CH₂— moiety is optionally replaced with —O—, —S— or —NH— and wherein said bridge is optionally substituted with at least one substituent R_{1g};
R₁ₑ and R_{1f} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, —C₂₋₈alkenyl, —C₂₋₈alkynyl, C₁₋₈alkoxy-C₁₋₈alkyl-, 3- to 8-membered heterocyclyl, phenyl or 5- to 12-membered heteroaryl;
R_{1g}, at each occurrence, is independently —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, phenyl, or 5- to 12-membered heteroaryl; preferably, R₁ₐ, R_{1b}, R₂ₐ, R_{2b}, R₃ₐ, R_{3b}, R_{1c}, R_{1d}, R_{2c}, R_{2d}, R_{3c}, R_{3d}, R_{Y1a}, R_{Y1b} and R_{Y2}, if present, are each independently hydrogen, —F, —Cl, —Br, —I, methyl, ethyl, propyl, butyl, cyclopropyl, cyclobutyl, cyclopentyl, cyclohexyl, —NH₂, —NHCH₃, —NHC₂H₅, —NHC₃H₇, —NHC₄H₉, —NHC₅H₁₁; and one pair of (R₁ₐ and R_{1c}), (R₁ₐ and R_{2c}), (R₁ₐ and R_{3c}), (R₂ₐ and R_{1c}), (R₂ₐ and R_{2c}), (R₂ₐ and R_{3c}), (R₃ₐ and R_{1c}), (R₃ₐ and R_{2c}), (R₃ₐ and R_{3c}), (R_{Y1a} and R_{Y2}), and (R_{Y1b} and R_{Y2}) form a bridge containing one, two, three, or four —CH₂— moieties in addition to the two bridgeheads, wherein one of the —CH₂— moiety is optionally replaced with —O—, —S— or —NH—.

Aspect 4: In some embodiments, wherein the ring

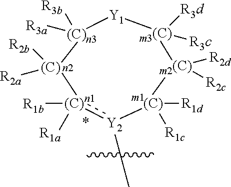

is a bridged bicyclic ring.

Aspect 5: In some embodiments, wherein
a. Y₁ is NH, Y₂ is N, [symbol] is a single bond, R₃ₐ and R_{3c} form a bridge containing one, two, or three —CH₂— moieties in addition to the two bridgeheads (nitrogen), wherein one of the —CH₂— moiety is optionally replaced with —O—, —S— or —NH— and wherein said bridge is optionally substituted with halogen (preferably F) or alkyl, and n3=m3=n1=m1=1 and n2=m2=0; or
b. Y₁ is NH, Y₂ is N, [symbol] is a single bond, R₂ₐ and R_{2c} form a bridge containing one, two, or three —CH₂— moieties in addition to the two bridgeheads (nitrogen), wherein one of the —CH₂— moiety is optionally replaced with —O—, —S— or —NH—, and n1=m1=n2=m2=n3=m3=1; or
c. Y₁ is NH, Y₂ is N, [symbol] is a single bond, R₁ₐ and R_{1c} form a bridge containing one, two, or three —CH₂— moieties in addition to the two bridgeheads (nitrogen), wherein one of the —CH₂— moiety is optionally replaced with —O—, —S— or —NH—, and n3=m3=n1=m1=1 and n2=m2=0; or
d. Y₁ is NH, Y₂ is N, [symbol] is a single bond, R₁ₐ and R_{1c} form a bridge containing one, two, or three —CH₂— moieties in addition to the two bridgeheads (nitrogen), wherein one of the —CH₂— moiety is optionally replaced with —O—, —S— or —NH—, and n2=m2=n3=m3=0 and n1=m1=1; or
e. Y₁ is NH, Y₂ is CH, [symbol] is a single bond, or Y₂ is C, [symbol] is a double bond, R₂ₐ and R_{1c} form a bridge containing one, two, or three —CH₂— moieties in addition to the two bridgeheads (one nitrogen and one carbon atom), wherein one of the —CH₂— moiety is optionally replaced with —O—, —S— or —NH—, and n1=n2=n3=m1=1, and m2=m3=0; or
f. Y₁ is NH, Y₂ is CH, [symbol] is a single bond, or Y₂ is C, [symbol] is a double bond, R₃ₐ and R_{3c} form a bridge containing one, two, or three —CH₂— moieties in addition to the two bridgeheads (one nitrogen and one carbon atom), wherein one of the —CH₂— moiety is optionally replaced with —O—, —S— or —NH—, and n1=m1=n3=m3=1, and n2=m2=0; or n3=m3=m1=1, and n1=n2=m2=0; or
g. Y₁ is CH, Y₂ is CH, [symbol] is a single bond, R_{Y1a} and R_{Y2} form a bridge containing one, two, or three —CH₂— moieties in addition to the two bridgeheads (two carbon atoms), wherein one of the —CH₂— moiety is optionally replaced with —O—, —S— or —NH—, and n1=m1=n3=m3=0, and n2=m2=1; or
h. Y₁ is NH, Y₂ is N, [symbol] is a single bond, R₃ₐ and R_{1c} form a bridge containing one, two, or three —CH₂— moieties in addition to the two bridgeheads (nitrogen), wherein one of the —CH₂— moiety is optionally replaced with —O—, —S— or —NH—, n1=m1=n3=m3=1, and n2=m2=0; or
i. Y₁ is NH, Y₂ is —CH(NH₂), [symbol] is a single bond, R₁ₐ and R_{1c} form a bridge containing one, two, or three —CH₂— moieties in addition to the two bridgeheads (one nitrogen and one carbon atom), wherein one of the —CH₂— moiety is optionally replaced with —O—, —S— or —NH—, n1=m1=n3=1, and n2=m2=m3=0.

Aspect 6: In some embodiments, wherein the ring

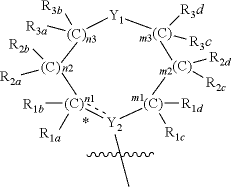

is

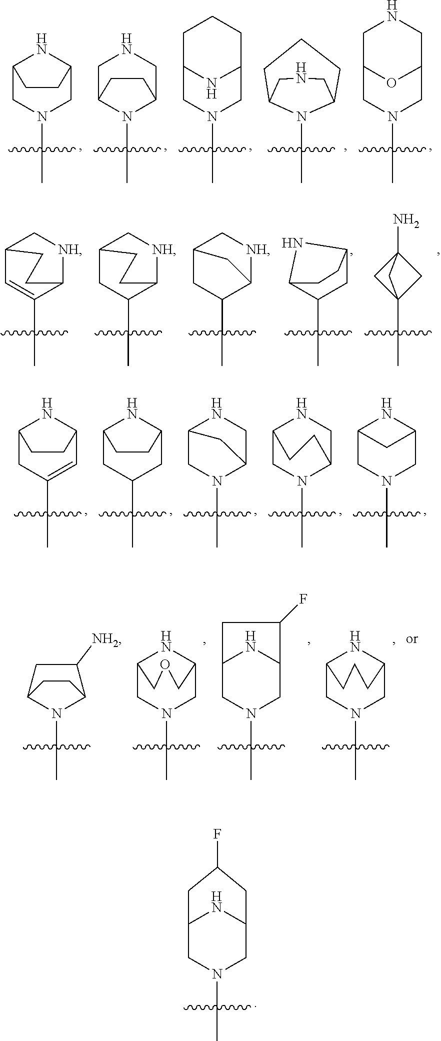

Aspect 6a: In some embodiments, wherein the ring

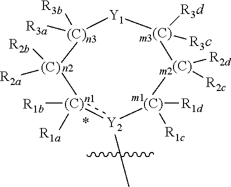

is

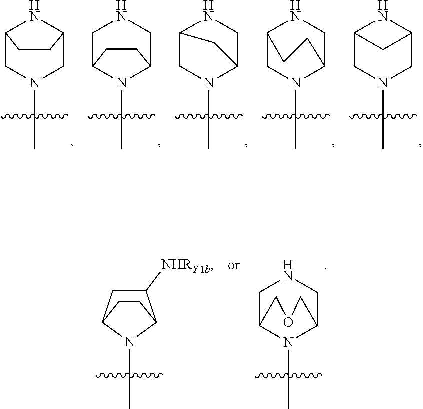

Aspect 6b: In some embodiments, wherein the ring

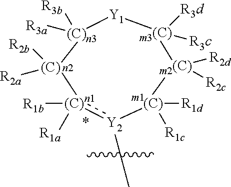

is

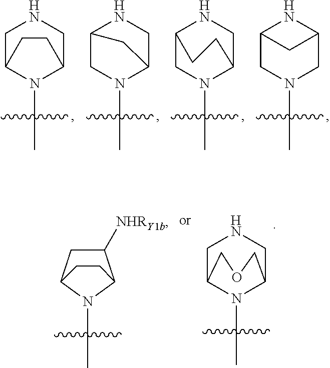

Aspect 6c: In some embodiments, wherein the ring

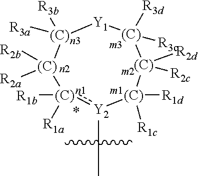

is

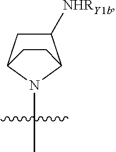

R_{Y1b} is —C₁₋₅alkyl is optionally substituted with fluoro or alkoxyl.

Aspect 7: In some embodiments,
wherein [symbol] is a double bond, X₁ is N, X₂ is C; or [symbol] is a sing bond, X₁ is C═O, X₂ is N.

Aspect 8: In some embodiments, wherein R₆ is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl, hexynyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, —CN, oxo, —OR₆ₐ, —SR₆ₐ or —NR₆ₐR_{6b}; each of methyl, ethyl, propyl, butyl, pentyl, hexyl, heptyl, octyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl, hexynyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with at least one substituent R_{6c};
R₆ₐ and R_{6b} are each independently selected from hydrogen, methyl, ethyl, propyl, butyl, pentyl, hexyl, heptyl, octyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl; wherein each of methyl, ethyl, propyl, butyl, pentyl, hexyl, heptyl, octyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R_{6d};
R_{6c}, at each occurrence, is independently —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; or two R₆ together with the atoms to which they are attached, form a 5- or 6-membered unsaturated (preferred aromatic) or saturated ring, said ring comprising 0, 1, 2, or 3 heteroatoms independently selected from nitrogen, oxygen or sulfur; said ring is optionally substituted with at least one substituent R_{6d};
R_{6d} is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CN, oxo, —OR₆ₑ or —NR₆ₑR_{6f}; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R_{6g};
R₆ₑ and R_{6f} are each independently selected from hydrogen, methyl, ethyl, propyl, butyl, pentyl, hexyl, heptyl, octyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl;
R_{6g}, at each occurrence, is independently —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, phenyl, or 5- to 12-membered heteroaryl;
preferably, R₆ is —OH, —NH₂, —F, —Cl, —Br, —I, —CH₃, —CH₂CH₃, —CH═CH, —C═CH, —CH₂CH₂CH₃, —CF₃, —CHF₂, —CH₂F, —CF₂CH₃, —CF₂CF₃, —OCHF₂, —OCF₃, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl.

Aspect 9: In some embodiments, wherein R₄, R₅ and R₇ are each independently hydrogen, —F, —Cl, —Br, —I, methyl, ethyl, propyl, butyl, pentyl, hexyl, heptyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl, hexynyl, —CN, oxo, —OR₄ₐ, —SR₄ₐ or —NR₄ₐR_{4b}; each of methyl, ethyl, propyl, butyl, pentyl, hexyl, heptyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl or hexynyl is optionally substituted with at least one substituent R_{4c},
R₄ₐ and R_{4b} are each independently selected from hydrogen, methyl, ethyl, propyl, butyl, pentyl, hexyl, heptyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl, hexynyl; wherein each of methyl, ethyl, propyl, butyl, pentyl, hexyl, heptyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl or hexynyl is optionally substituted with at least one substituent R_{4d};
R_{4c} and R_{4d}, at each occurrence, are each independently —F, —Cl, —Br, —I, methyl, ethyl, propyl, butyl, pentyl, hexyl, heptyl, vinyl, propylenyl, allyl, butenyl, butadienyl, pentenyl, pentadienyl, hexenyl, hexadienyl, ethynyl, propynyl, butynyl, pentynyl, hexynyl, cyclopropyl, cyclobutyl, cyclopentyl, cyclohexyl, cycloheptyl, cyclooctyl, 3- to 8-membered heterocyclyl, —C₆-C₁₂aryl, or 5- to 12-membered heteroaryl.

Aspect 10. The compound of any one of Aspects 1-9, wherein R₄, R₅ and R₇ are each independently hydrogen, —F, —Cl, —Br, —I, methyl, ethyl, propyl, iso-propyl, iso-butyl, tert-butyl, —CF₃, —CHF₂, —CH₂F, —OCHF₂, —OCF₃, cyclopropyl, cyclobutyl, cyclopentyl, vinyl(—CH═CH), propylenyl(such as —C(CH₃)═CH), allyl, ethynyl, propynyl, —Sme, —SCF₃, —CF₂CH₃, —SCHF₂, —CF═CH, or —SCH₂F.

Aspect 10a: In some embodiments, wherein wherein R₄, R₅ and R₇ are each independently hydrogen, —F, —Cl, —Br, —I, methyl, ethyl, propyl, iso-propyl, iso-butyl, tert-butyl, —CF₃, —CHF₂, —CH₂F, —OCHF₂, —OCF₃, cyclopropyl, methylcyclopropyl, fluorocyclopropyl, difluorocyclopropyl, fluoromethylcyclopropyl, cyclobutyl, cyclopentyl, vinyl(—CH═CH), propylenyl(such as —C(CH₃)═CH), allyl, ethynyl, propynyl, —SMe, —SCF₃, —CF₂CH₃, —CH(OH)CH₃—CHFCH₃, —SCHF₂, —CF═CH, CH₂CF₃—, —CH₂CF₂, or —SCH₂F.

Aspect 11. The compound of any one of Aspects 1-10, wherein the

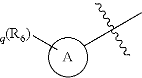

moiety is an aryl group selected from phenyl or naphthyl substituted with one or two R₆. In some embodiments, R₆ is selected from —CN, OH, —CF₃, —CHF₂, —CH₂F, —CF₂CH₃, —CF₂CF₃, —OCHF₂, —OCF₃, vinyl(—CH═CH), -propylenyl (such as —C(CH₃)═CH), —CF═CH, aryl, heteroaryl, ethynyl, propynyl, butynyl, pentynyl, hexynyl, cyclopropyl, cyclobutyl, cyclopentyl, cyclohexyl, cycloheptyl, cyclooctyl, methyl, ethyl, propyl, butyl, pentyl, hexyl, heptyl, octyl, vinyl, propylenyl, allyl, butenyl, butadienyl, pentenyl, pentadienyl, hexenyl, hexadienyl, methoxy, ethoxy, propoxy, isopropoxy, butoxy, tert-butoxy, pentoxy, or hexoxy.

Aspect 11a: In some embodiments, wherein the

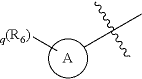

moiety is an aryl group selected from phenyl or naphthyl substituted with one or two R₆. In some embodiments, R₆ is selected from —CN, OH, —CF₃, —CHF₂, —CH₂F, —CF₂CH₃, —CF₂CF₃, —OCHF₂, —OCF₃, vinyl(—CH═CH), -propylenyl(such as —C(CH₃)═CH), —CF═CH, aryl, heteroaryl, ethynyl, propynyl, butynyl, pentynyl, hexynyl, cyclopropyl, methylcyclopropyl, fluorocyclopropyl, difluorocyclopropyl, fluoromethylcyclopropyl, cyclobutyl, cyclopentyl, cyclohexyl, cycloheptyl, cyclooctyl, methyl, ethyl, propyl, butyl, pentyl, hexyl, heptyl, octyl, vinyl, propylenyl, allyl, butenyl, butadienyl, pentenyl, pentadienyl, hexenyl, hexadienyl, methoxy, ethoxy, propoxy, isopropoxy, butoxy, tert-butoxy, pentoxy, or hexoxy.

Aspect 12. The compound of any one of Aspects 1-10, wherein the

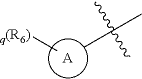

moiety is a 5- to 7-membered monocyclic heteroaryl or 8- to 12-membered bicyclic heteroaryl group substituted with one or two R6. In some embodiments, R₆ is selected from F, Cl, Br, C(CH₃)═CH or —CH═CH, —CF═CH, —CN, OH, NH2, —CF3, —CHF2, —CH2F, —CF2CH3, —CF2CF3, —OCHF₂, —OCF₃, aryl, ethynyl, propynyl, butynyl, pentynyl, hexynyl, cyclopropyl, cyclobutyl, cyclopentyl, cyclohexyl, cycloheptyl, cyclooctyl, methyl, ethyl, propyl, butyl, pentyl, hexyl, heptyl, octyl, vinyl, propylenyl, allyl, butenyl, butadienyl, pentenyl, pentadienyl, hexenyl, hexadienyl, methoxy, ethoxy, propoxy, isopropoxy, butoxy, tert-butoxy, pentoxy, or hexoxy. In some preferred embodiments, ring A is pyridyl, benzothiazolyl, quinolinyl, isoquinolinyl, pyrazolopyridinyl, benzoimidazolyl, quinazolinyl, or quinazolinyl.

Aspect 12a: In some embodiments, wherein the

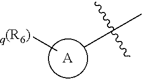

moiety is a 5- to 7-membered monocyclic heteroaryl or 8- to 12-membered bicyclic heteroaryl group substituted with one or two R₆. In some embodiments, R₆ is selected from F, Cl, Br, C(CH₃)═CH or —CH═CH, —CF═CH, —CN, OH, NH₂, —CF₃, —CHF₂, —CH₂F, —CF₂CH₃, —CF₂CF₃, —OCHF₂, —OCF₃, aryl, ethynyl, propynyl, butynyl, pentynyl, hexynyl, cyclopropyl, methylcyclopropyl, fluorocyclopropyl, difluorocyclopropyl, fluoromethylcyclopropyl, cyclobutyl, cyclopentyl, cyclohexyl, cycloheptyl, cyclooctyl, methyl, ethyl, propyl, butyl, pentyl, hexyl, heptyl, octyl, vinyl, propylenyl, allyl, butenyl, butadienyl, pentenyl, pentadienyl, hexenyl, hexadienyl, methoxy, ethoxy, propoxy, isopropoxy, butoxy, tert-butoxy, pentoxy, or hexoxy. In some preferred embodiments, ring A is pyridyl, benzothiazolyl, quinolinyl, isoquinolinyl, pyrazolopyridinyl, benzoimidazolyl, quinazolinyl, or quinazolinyl.

Aspect 13. The compound of any one of Aspects 1-12, wherein the

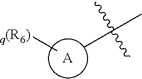

moiety is

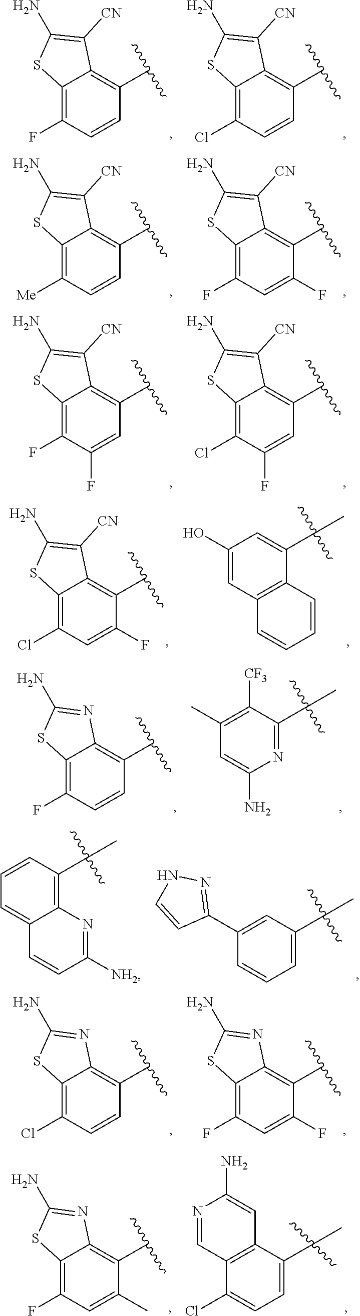

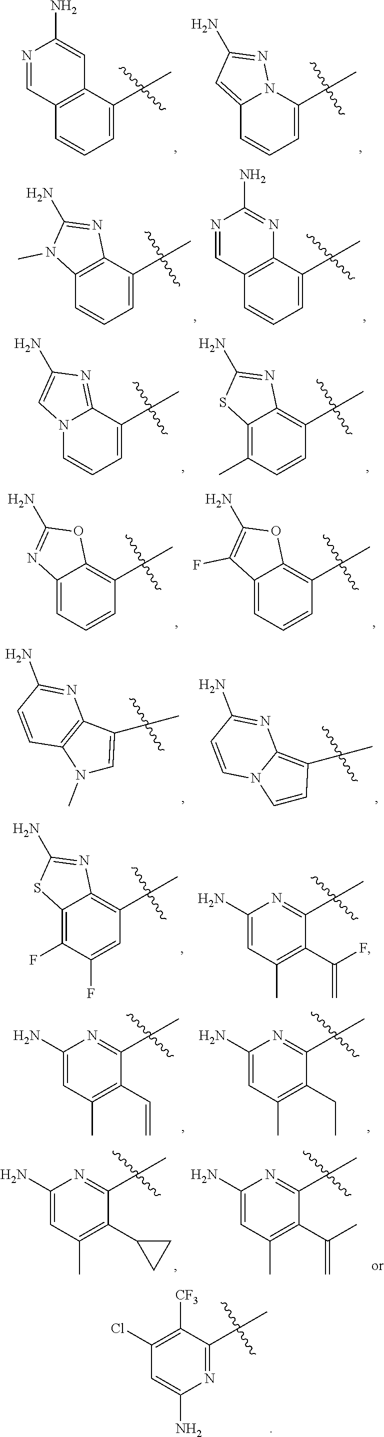

Aspect 13a: In some embodiments, wherein the

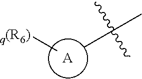

moiety is

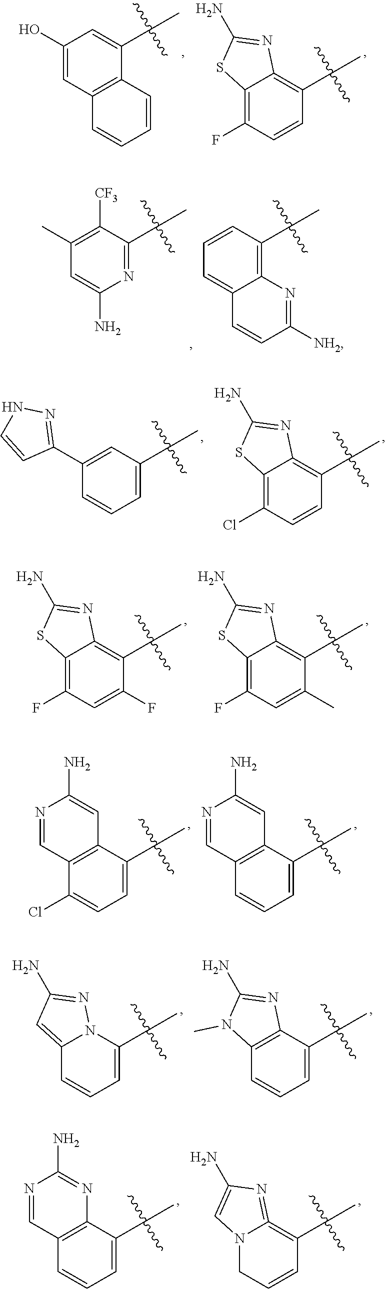

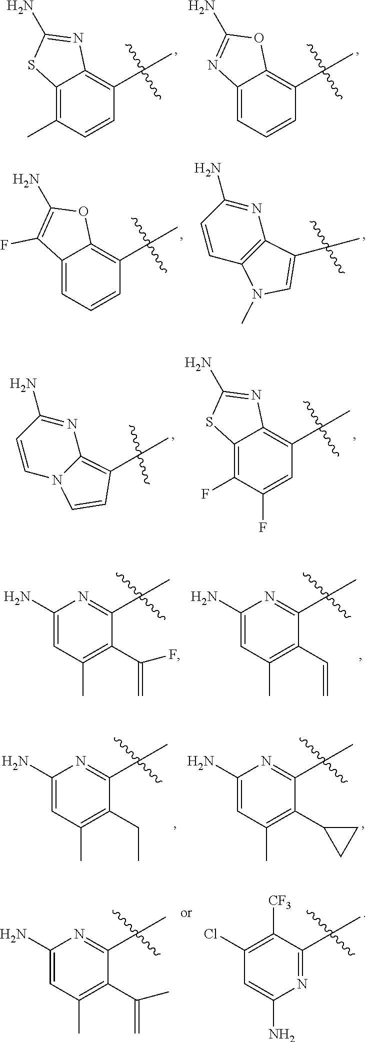

In some embodiments, wherein the

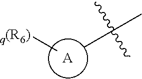

moiety is

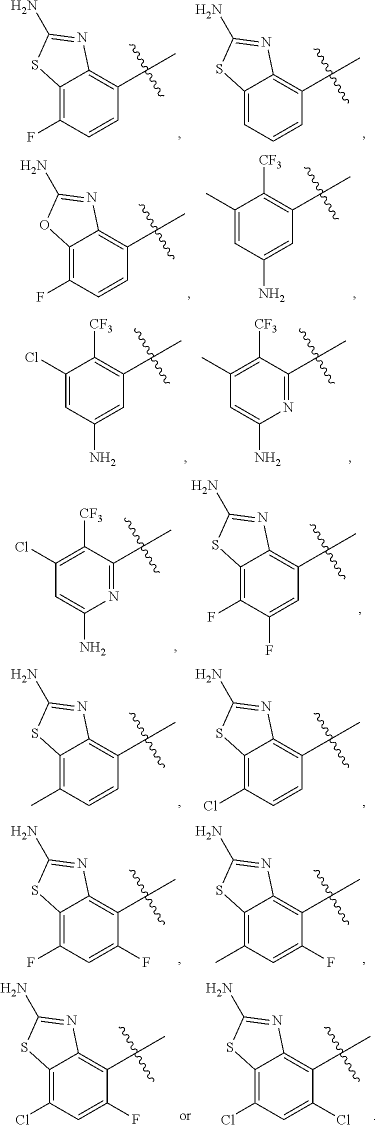

Aspect 13b. The compound of any one of Aspects 1-12, wherein the

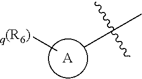

moiety is

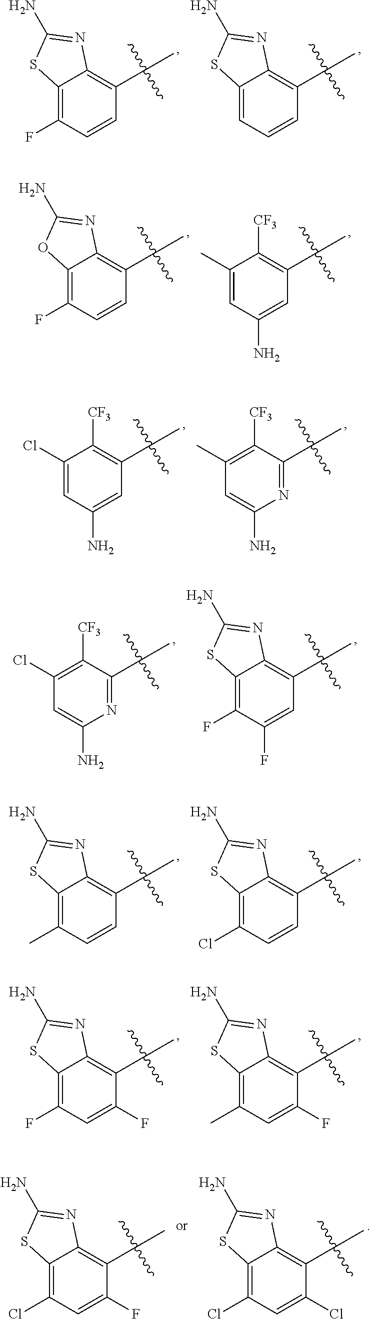

Aspect 14. The compound of any one of Aspects 1-13, wherein X₁ is N and X₂ is C.

Aspect 14a: In some embodiments, wherein [symbol] is a double bond, X₁ is N and X₂ is C.

Aspect 15. The compound of any one of Aspects 1-14, wherein the

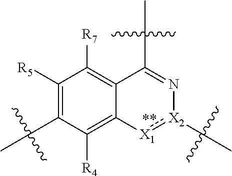

moiety is

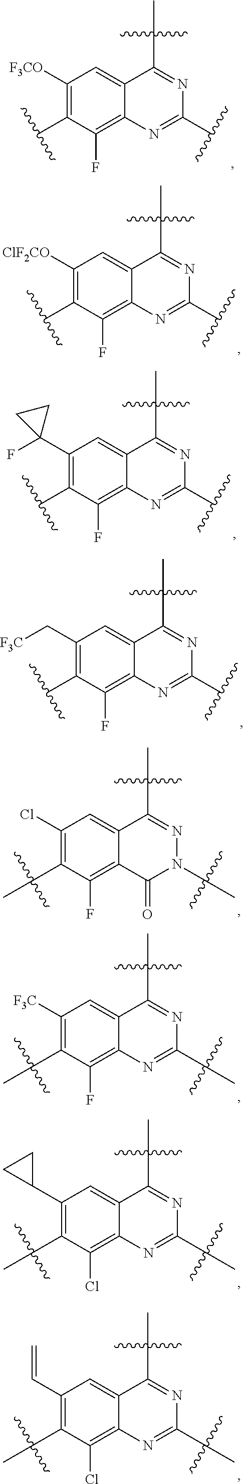

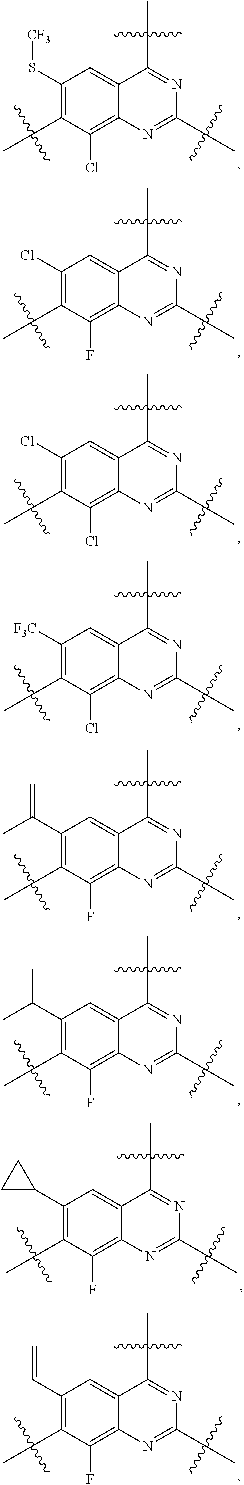

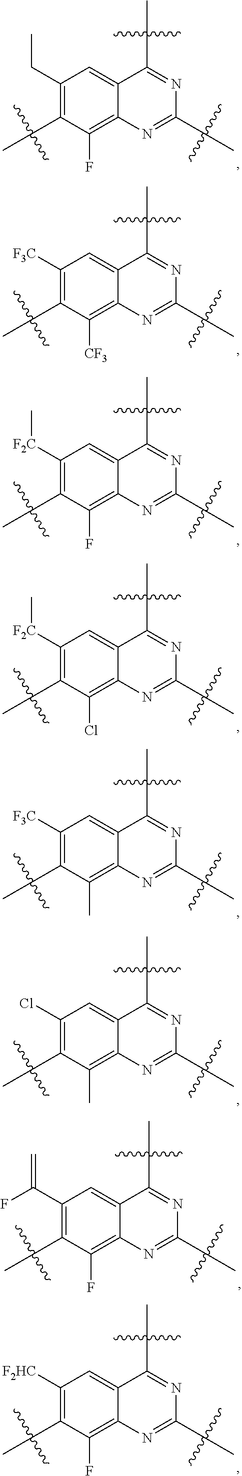

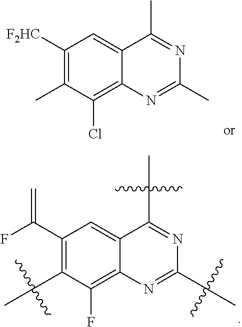 [SMILES: Cc1nc(C)c2cc(C(F)F)c(C)c(Cl)c2n1 ; *c1nc(*)c2cc(C(=C)F)c(*)c(F)c2n1]

Aspect 15a: In some embodiments wherein the

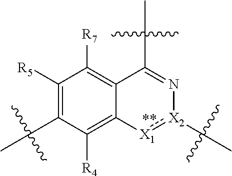

moiety is,

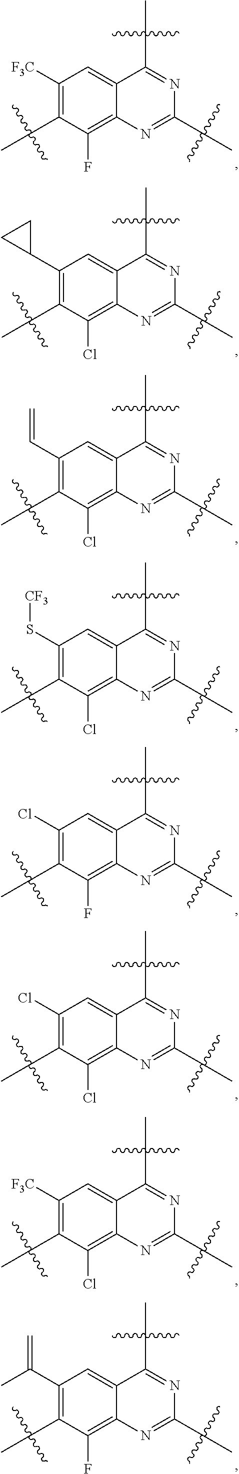

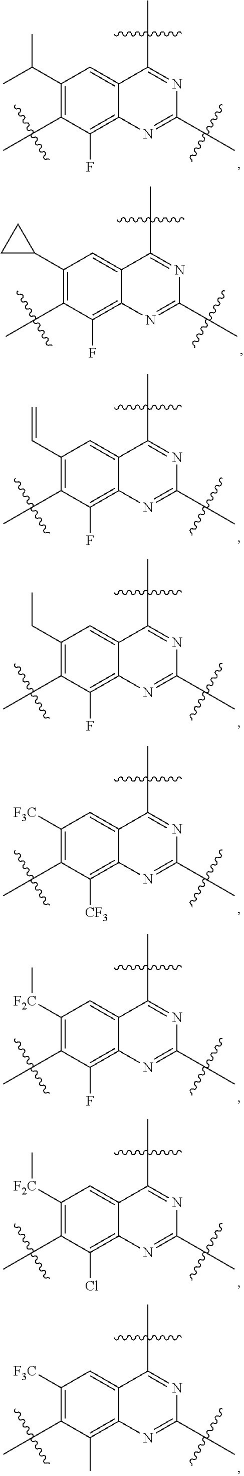

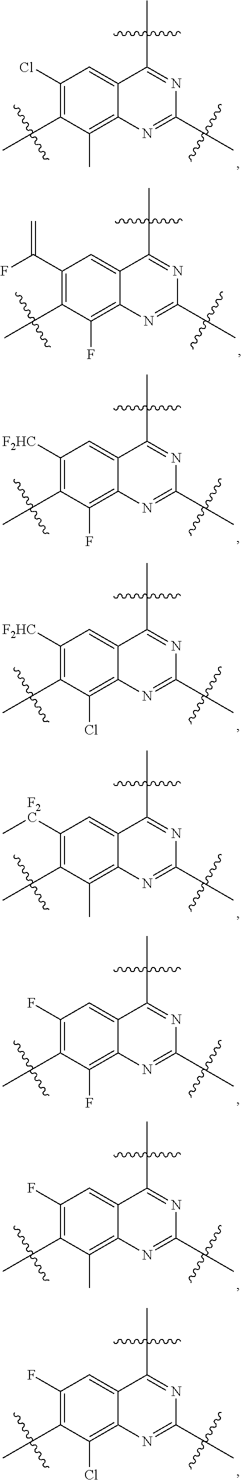

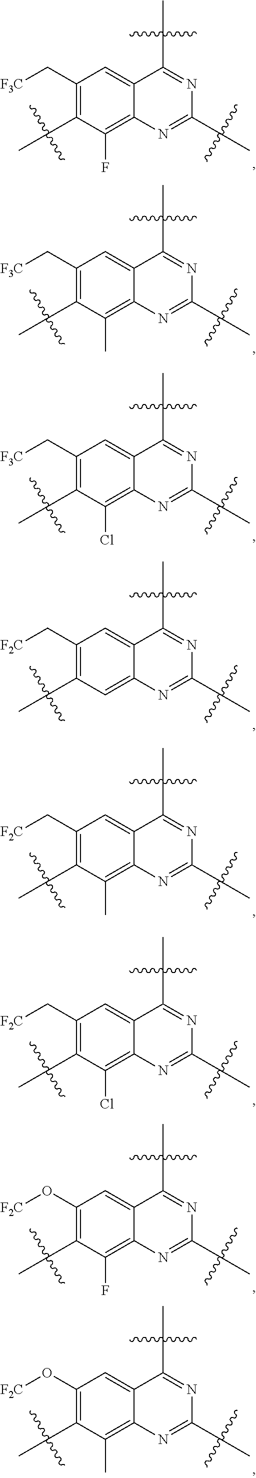

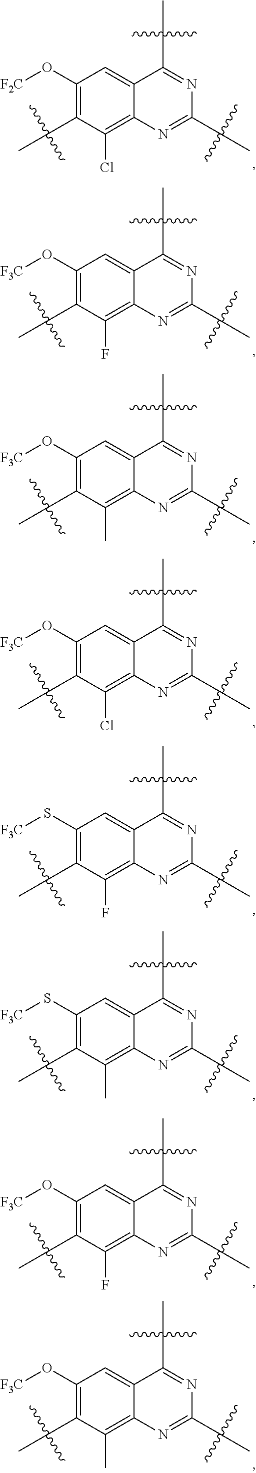

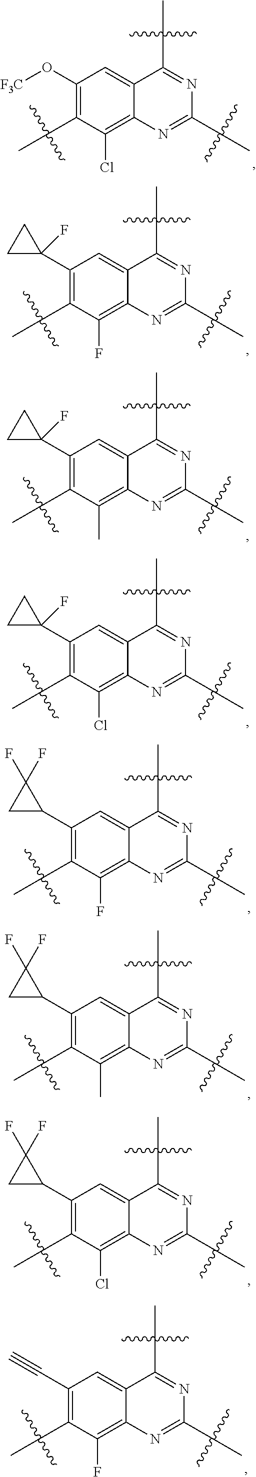

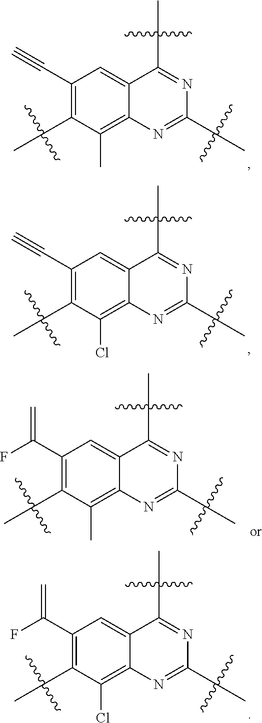 [SMILES: *c1nc(*)c2cc(C#C)c(*)c(C)c2n1 ; *c1nc(*)c2cc(C#C)c(*)c(Cl)c2n1 ; *c1nc(*)c2cc(C(=C)F)c(*)c(C)c2n1 ; *c1nc(*)c2cc(C(=C)F)c(*)c(Cl)c2n1]

Aspect 16: In some embodiments, wherein L₁ is a single bond, —O—, —S—, —NR^{L1a}—, —C(O)—, —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH₂CH₂CH₂CH₂CH₂CH₂CH₂—, —CH₂CH₂CH₂CH₂CH₂CH₂CH₂CH₂—, —C₃-C₈cycloalkylene-, *^{L1}—O—CH₂—**^{L1}, *^{L1}—O—CH₂CH₂—**^{L1}, *^{L1}—O—CH₂CH₂CH₂—**^{L1}, *^{L1}—CH₂CH₂CH₂CH₂—**^{L1}, *^{L1}—O—CH₂CH₂CH₂CH₂CH₂—**^{L1}, *^{L1}—O—CH₂CH₂CH₂CH₂CH₂CH₂—**^{L1}, *^{L1}—CH₂CH₂CH₂CH₂CH₂CH₂CH₂—**^{L1}, *^{L1}—O—CH₂CH₂CH₂CH₂CH₂CH₂CH₂CH₂—**^{L1}, *^{L1}—CH₂—O—**^{L1}, *^{L1}—CH₂CH₂—O—**^{L1}, *^{L1}—CH₂CH₂CH₂—O—**^{L1}, *^{L1}—CH₂CH₂CH₂CH₂—O—**^{L1}, *^{L1}—CH₂CH₂CH₂CH₂CH₂—O—**^{L1}, *^{L1}—CH₂CH₂CH₂CH₂CH₂CH₂—O—**^{L1}, *^{L1}—CH₂CH₂CH₂CH₂CH₂CH₂CH₂—O—**^{L1}, *^{L1}—CH₂CH₂CH₂CH₂CH₂CH₂CH₂CH₂—O—**^{L1}, *^{L1}—S—CH₂—**^{L1}, *^{L1}—S—CH₂CH₂—**^{L1}, *^{L1}—S—CH₂CH₂CH₂—**^{L1}, *^{L1}—S—CH₂CH₂CH₂CH₂—**^{L1}, *^{L1}—S—CH₂CH₂CH₂CH₂CH₂—**^{L1}, *^{L1}—S—CH₂CH₂CH₂CH₂CH₂CH₂—**^{L1}, *^{L1}—S—CH₂CH₂CH₂CH₂CH₂CH₂CH₂—**^{L1}, *^{L1}—S—CH₂CH₂CH₂CH₂CH₂CH₂CH₂CH₂—**^{L1}, *^{L1}—CH₂—S—**^{L1}, *^{L1}—CH₂CH₂—S—**^{L1}, *^{L1}—CH₂CH₂CH₂—S—**^{L1}, *^{L1}—CH₂CH₂CH₂CH₂—S—**^{L1}, *^{L1}—CH₂CH₂CH₂CH₂CH₂—S—**^{L1}, *^{L1}—CH₂CH₂CH₂CH₂CH₂CH₂—S—**^{L1}, *^{L1}—CH₂CH₂CH₂CH₂CH₂CH₂CH₂—S—**^{L1}, *^{L1}—CH₂CH₂CH₂CH₂CH₂CH₂CH₂CH₂—S—**^{L1}, *^{L1}—O—C₃-C₈cycloalkylene-**^{L1}, *^{L1}—C₃-C₈cycloalkylene-O—**^{L1}, *^{L1}—S—C₃-C₈cycloalkylene-**^{L1}, *^{L1}—C₃-C₈cycloalkylene-S—**^{L1}, *^{L1}—O—CH₂—NR^{L1a}—**^{L1}, *^{L1}—O—CH₂CH₂—NR^{L1a}—**^{L1}, *^{L1}—O—CH₂CH₂CH₂—NR^{L1a}—**^{L1}, *^{L1}—O—CH₂CH₂CH₂CH₂—NR^{L1a}—**^{L1}, *^{L1}—CH₂CH₂CH₂CH₂CH₂—NR^{L1a}—**^{L1}, *^{L1}—O—CH₂CH₂CH₂CH₂CH₂CH₂—NR^{L1a}—**^{L1}, *^{L1}—CH₂CH₂CH₂CH₂CH₂CH₂CH₂—NR^{L1a}—**^{L1}, *^{L1}—CH₂CH₂CH₂CH₂CH₂CH₂CH₂CH₂—NR^{L1a}—**^{L1}, *^{L1}—NR^{L1a}—CH₂—O—**^{L1}, *^{L1}—NR^{L1a}—CH₂CH₂—O—**^{L1}, *^{L1}—NR^{L1a}—CH₂CH₂CH₂—O—**^{L1}, *^{L1}—NR^{L1a}—CH₂CH₂CH₂CH₂—O—**^{L1}, *^{L1}—NR^{L1a}—CH₂CH₂CH₂CH₂CH₂—O—**^{L1}, *^{L1}—NR^{L1a}—CH₂CH₂CH₂CH₂CH₂CH₂—O—**^{L1}, *^{L1}—NR^{L1a}—CH₂CH₂CH₂CH₂CH₂CH₂CH—O—**^{L1}, *^{L1}—NR^{L1a}—CH₂CH₂CH₂CH₂CH₂CH₂CH₂CH₂—**^{L1}, *^{L1}—S—CH₂—NR^{L1a}—**^{L1}, *^{L1}—S—CH₂CH₂—NR^{L1a}—**^{L1}, *^{L1}—S—CH₂CH₂CH₂—NR^{L1a}—**^{L1}, *^{L1}—S—CH₂CH₂CH₂CH₂—NR^{L1a}—**^{L1}, *^{L1}—S—CH₂CH₂CH₂CH₂CH₂—NR^{L1a}—**^{L1}, *^{L1}—S—CH₂CH₂CH₂CH₂CH₂CH₂—NR^{L1a}—**^{L1}, *^{L1}—S—CH₂CH₂CH₂CH₂CH₂CH₂CH₂—NR^{L1a}—**^{L1}, *^{L1}—S—CH₂CH₂CH₂CH₂CH₂CH₂CH₂CH₂—NR^{L1a}—**^{L1}, *^{L1}—NR^{L1a}—CH₂—S—**^{L1}, *^{L1}—NR^{L1a}—CH₂CH₂—S—**^{L1}, *^{L1}—NR^{L1a}—CH₂CH₂CH₂—S—**^{L1}, *NR^{L1a}—CH₂CH₂CH₂CH₂—S—**^{L1}, *^{L1}—NR^{L1a}—CH₂CH₂CH₂CH₂CH₂—S—**^{L1}, *^{L1}—NR^{L1a}—CH₂CH₂CH₂CH₂CH₂CH₂—S—**^{L1}, *^{L1}—NR^{L1a}—CH₂CH₂CH₂CH₂CH₂CH₂CH₂—S—**^{L1}, *^{L1}—NR^{L1a}—CH₂CH₂CH₂CH₂CH₂CH₂CH₂CH₂—S—**^{L1}, *^{L1}—O—CH₂—CO—**^{L1}, *^{L1}—O—CH₂CH₂—CO—**^{L1}, *^{L1}—O—CH₂CH₂CH₂—CO—**^{L1}, *^{L1}—O—CH₂CH₂CH₂CH₂—CO—**^{L1}, *^{L1}—O—CH₂CH₂CH₂CH₂CH₂—CO—**^{L1}, *^{L1}—O—CH₂CH₂CH₂CH₂CH₂CH₂—CO—**^{L1}, *^{L1}—O—CH₂CH₂CH₂CH₂CH₂CH₂CH₂—CO—**^{L1}, *^{L1}—O—CH₂CH₂CH₂CH₂CH₂CH₂CH₂CH₂—CO—**^{L1}, *^{L1}—CO—CH₂—O—**^{L1}—CO—CH₂CH₂—O—**^{L1}, *^{L1}—CO—CH₂CH₂CH₂—O—**^{L1}, *^{L1}—CO—CH₂CH₂CH₂CH₂—O—**^{L1}, *^{L1}—CO—CH₂CH₂CH₂CH₂CH₂—O—**^{L1}, *^{L1}—CO—CH₂CH₂CH₂CH₂CH₂CH₂—O—**^{L1}, *^{L1}—CO—CH₂CH₂CH₂CH₂CH₂CH₂CH₂—O—**^{L1}, *^{L1}—CO—CH₂CH₂CH₂CH₂CH₂CH₂CH₂CH₂—O—**^{L1}, *^{L1}—S—CH₂—CO—**^{L1}, *^{L1}—S—CH₂CH₂—CO—**^{L1}, *^{L1}—S—CH₂CH₂CH₂—CO—**^{L1}, *^{L1}—S—CH₂CH₂CH₂CH₂—CO—**^{L1}, *^{L1}—S—CH₂CH₂CH₂CH₂CH₂—CO—**^{L1}, *^{L1}—S—CH₂CH₂CH₂CH₂CH₂CH₂—CO—**^{L1}, *^{L1}—S—CH₂CH₂CH₂CH₂CH₂CH₂CH₂—CO—**^{L1}, *^{L1}—S—CH₂CH₂CH₂CH₂CH₂CH₂CH₂CH₂—CO—**^{L1}, *^{L1}—CO—CH₂—S—**^{L1}, *^{L1}—CO—CH₂CH₂—S—**^{L1}, *^{L1}—CO—CH₂CH₂CH₂—S—**^{L1}, *^{L1}—CO—CH₂CH₂CH₂CH₂—S—**^{L1}, *^{L1}—CO—CH₂CH₂CH₂CH₂CH₂—S—**^{L1}, *^{L1}—CO—CH₂CH₂CH₂CH₂CH₂CH₂—S—**^{L1}, *^{L1}—CO—CH₂CH₂CH₂CH₂CH₂CH₂CH₂—S—**^{L1}, *^{L1}—CO—CH₂CH₂CH₂CH₂CH₂CH₂CH₂CH₂—S—**^{L1}, *^{L1}—C(O)—CH₂—**^{L1}, *^{L1}—C(O)—CH₂CH₂—**^{L1}, *^{L1}—C(O)—CH₂CH₂CH₂—**^{L1}, *^{L1}—C(O)—CH₂CH₂CH₂CH₂—**^{L1}, *^{L1}—C(O)—CH₂CH₂CH₂CH₂CH₂—**^{L1}, *^{L1}—C(O)—CH₂CH₂CH₂CH₂CH₂CH₂—**^{L1}, *^{L1}—CH₂CH₂CH₂CH₂CH₂CH₂CH₂—**^{L1}, *^{L1}—C(O)—CH₂CH₂CH₂CH₂CH₂CH₂CH₂CH₂—**^{L1}, *^{L1}—CH₂—C(O)—**^{L1}, *^{L1}—CH₂CH₂—C(O)—**^{L1}, *^{L1}—CH₂CH₂CH₂—C(O)—**^{L1}, *^{L1}—CH₂CH₂CH₂CH₂—C(O)—**^{L1}, *^{L1}—CH₂CH₂CH₂CH₂CH₂—C(O)—**^{L1}, *^{L1}—CH₂CH₂CH₂CH₂CH₂CH₂—C(O)—**^{L1}, *^{L1}—CH₂CH₂CH₂CH₂CH₂CH₂CH₂—C(O)—**^{L1}, *^{L1}—CH₂CH₂CH₂CH₂CH₂CH₂CH₂CH₂—C(O)—**^{L1}, *^{L1}—CH₂—NR^{L1a}—**^{L1}, *^{L1}—CH₂CH₂—NR^{L1a}—**^{L1}, *^{L1}—CH₂CH₂CH₂—NR^{L1a}—**^{L1}, *^{L1}—CH₂CH₂CH₂CH₂—NR^{L1a}—**^{L1}, *^{L1}—CH₂CH₂CH₂CH₂CH₂—NR^{L1a}—**^{L1}, *^{L1}—CH₂CH₂CH₂CH₂CH₂CH₂—NR^{L1a}—**^{L1}, *^{L1}—CH₂CH₂CH₂CH₂CH₂CH₂CH₂—NR^{L1a}—**^{L1}, *^{L1}—CH₂CH₂CH₂CH₂CH₂CH₂CH₂CH₂—NR^{L1a}—**^{L1}, *^{L1}—NR^{L1a}—CH₂—**^{L1}, *^{L1}—NR^{L1a}—CH₂CH₂—**^{L1}, *^{L1}—NR^{L1a}—CH₂CH₂CH₂—**^{L1}, *^{L1}—NR^{L1a}—CH₂CH₂CH₂CH₂—**^{L1}, *^{L1}—NR^{L1a}—CH₂CH₂CH₂CH₂CH₂—**^{L1}, *^{L1}—NR^{L1a}—CH₂CH₂CH₂CH₂CH₂CH₂—**^{L1}, *^{L1}—NR^{L1a}—CH₂CH₂CH₂CH₂CH₂CH₂CH₂—**^{L1}, *^{L1}—NR^{L1a}—CH₂CH₂CH₂CH₂CH₂CH₂CH₂CH₂—**^{L1}, *^{L1}—CH₂—C(O)NR^{L1a}—**^{L1}, *^{L1}—CH₂CH₂—C(O)NR^{L1a}—**^{L1}, *^{L1}—CH₂CH₂CH₂—C(O)NR^{L1a}—**^{L1}, *^{L1}—CH₂CH₂CH₂CH₂—C(O)NR^{L1a}—**^{L1}, *^{L1}—CH₂CH₂CH₂CH₂CH₂—C(O)NR^{L1a}—**^{L1}, *^{L1}—CH₂CH₂CH₂CH₂CH₂CH₂—C(O)NR^{L1a}—**^{L1}, *^{L1}—CH₂CH₂CH₂CH₂CH₂CH₂CH₂—C(O)NR^{L1a}—**^{L1}, *^{L1}—CH₂CH₂CH₂CH₂CH₂CH₂CH₂CH₂—C(O)NR^{L1a}—**^{L1}, *^{L1}—C(O)NR^{L1a}—CH₂—**^{L1}, *^{L1}—C(O)NR^{L1a}—CH₂CH₂—**^{L1}, *^{L1}—C(O)NR^{L1a}—CH₂CH₂CH₂—**^{L1}, *^{L1}—C(O)NR^{L1a}—CH₂CH₂CH₂CH₂—**^{L1}, *^{L1}—C(O)NR^{L1a}—CH₂CH₂CH₂CH₂CH₂—**^{L1}, *^{L1}—C(O)NR^{L1a}—CH₂CH₂CH₂CH₂CH₂CH₂—**^{L1}, *^{L1}—C(O)NR^{L1a}—CH₂CH₂CH₂CH₂CH₂CH₂CH₂—**^{L1}, *^{L1}—C(O)NR^{L1a}—CH₂CH₂CH₂CH₂CH₂CH₂CH₂CH₂—**^{L1}, *^{L1}—CH₂—NR^{L1a}C(O)—**^{L1}, *^{L1}—CH₂CH₂—NR^{L1a}C(O)—**^{L1}, *^{L1}—CH₂CH₂CH₂—NR^{L1a} C(O)—**^{L1}, *^{L1}—CH₂CH₂CH₂CH₂—NR^{L1a}C(O)—**^{L1}, *^{L1}—CH₂CH₂CH₂CH₂CH₂—NR^{L1a}C(O)—**^{L1}, *^{L1}—CH₂CH₂CH₂CH₂CH₂CH₂—NR^{L1a}C(O)—**^{L1}, *^{L1}—CH₂CH₂CH₂CH₂CH₂CH₂CH₂—NR^{L1a}C(O)—**^{L1}, *^{L1}—CH₂CH₂CH₂CH₂CH₂CH₂CH₂CH₂—NR^{L1a}C(O)—**^{L1}, *^{L1}—NR^{L1a}C(O)—CH₂—**^{L1}, *^{L1}—NR^{L1a}C(O)—CH₂CH₂—**^{L1}, *^{L1}—NR^{L1a}C(O)—CH₂CH₂CH₂—**^{L1}, *^{L1}—NR^{L1a}C(O)—CH₂CH₂CH₂CH₂—**^{L1}, *^{L1}—NR^{L1a}C(O)—CH₂CH₂CH₂CH₂CH₂—**^{L1}, *^{L1}—NR^{L1a}C(O)—CH₂CH₂CH₂CH₂CH₂CH₂—**^{L1}, *^{L1}—NR^{L1a}C(O)—CH₂CH₂CH₂CH₂CH₂CH₂CH₂—**^{L1}, *^{L1}—NR^{L1a}C(O)—CH₂CH₂CH₂CH₂CH₂CH₂CH₂CH₂—**^{L1}, *^{L1}—C(O)O—CH₂—**^{L1}, *^{L1}—C(O)O—CH₂CH₂—**^{L1}, *^{L1}—C(O)O—CH₂CH₂CH₂—**^{L1}, *^{L1}—C(O)O—CH₂CH₂CH₂CH₂—**^{L1}, *^{L1}—C(O)O—CH₂CH₂CH₂CH₂CH₂—**^{L1}, *^{L1}—C(O)O—CH₂CH₂CH₂CH₂CH₂CH₂—**^{L1}, *^{L1}—C(O)O CH₂CH₂CH₂CH₂CH₂CH₂CH₂—**^{L1}, *^{L1}—C(O)O—CH₂CH₂CH₂CH₂CH₂CH₂CH₂CH₂—**^{L1}, *^{L1}—CH₂—C(O)O—**^{L1}, *^{L1}—CH₂CH₂—C(O)O—**^{L1}, *^{L1}—CH₂CH₂CH₂—C(O)O—**^{L1}, *^{L1}—CH₂CH₂CH₂CH₂—C(O)O—**^{L1}, *^{L1}—CH₂CH₂CH₂CH₂CH₂—C(O)O—**^{L1}, *^{L1}—CH₂CH₂CH₂CH₂CH₂CH₂—C(O)O—**^{L1}, *^{L1}—CH₂CH₂CH₂CH₂CH₂CH₂CH₂—C(O)O—**^{L1}, *^{L1}—CH₂CH₂CH₂CH₂CH₂CH₂CH₂CH₂—C(O)O—**^{L1}, *^{L1}—OC(O)—CH₂—**^{L1}, *^{L1}—OC(O)—CH₂CH₂—**^{L1}, *^{L1}—OC(O)—CH₂CH₂CH₂—**^{L1}, *^{L1}—OC(O)—CH₂CH₂CH₂CH₂— **^{L1}, *^{L1}—OC(O)—CH₂CH₂CH₂CH₂CH₂—**^{L1}, *^{L1}—OC(O)—CH₂CH₂CH₂CH₂CH₂CH₂—**^{L1}, *^{L1}—OC(O)—CH₂CH₂CH₂CH₂CH₂CH₂CH₂—**^{L1}, *^{L1}—OC(O)—CH₂CH₂CH₂CH₂CH₂CH₂CH₂CH₂—**^{L1}, *^{L1}—CH₂—OC(O)—**^{L1}, *^{L1}—CH₂CH₂—OC(O)—**^{L1}, *^{L1}—CH₂CH₂CH₂—OC(O)—**^{L1}, *^{L1}—CH₂CH₂CH₂CH₂—OC(O)—**^{L1}, *^{L1}—CH₂CH₂CH₂CH₂CH₂—OC(O)—**^{L1}, *^{L1}—CH₂CH₂CH₂CH₂CH₂CH₂—OC(O)—**^{L1}, *^{L1}—CH₂CH₂CH₂CH₂CH₂CH₂CH₂—OC(O)—**^{L1}, *^{L1}—CH₂CH₂CH₂CH₂CH₂CH₂CH₂CH₂—OC(O)—**^{L1},

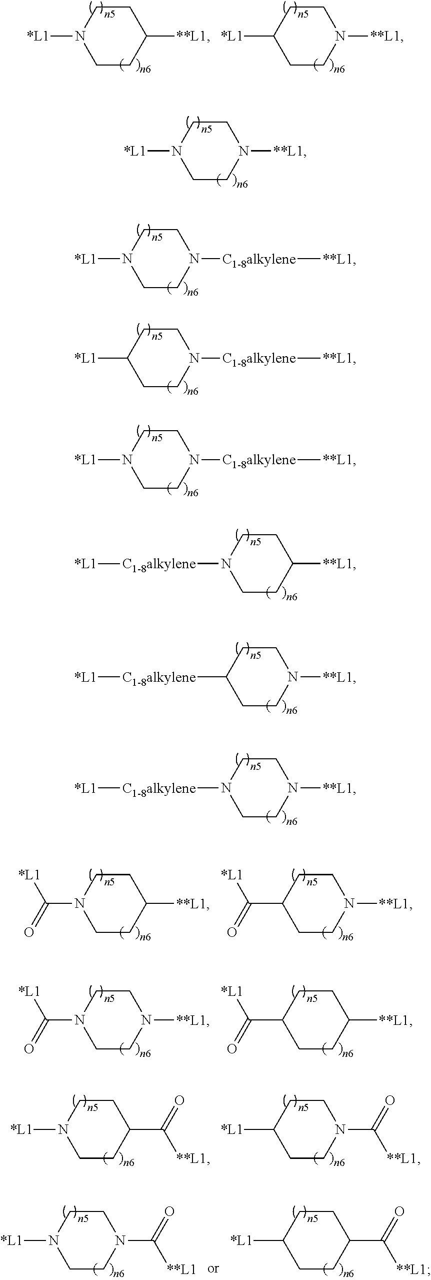

each of said —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH₂CH₂CH₂CH₂CH₂CH₂CH₂—, —CH₂CH₂CH₂CH₂CH₂CH₂CH₂CH₂—, —C₃-C₈cycloalkylene-, *^{L1}—O—CH₂—**^{L1}, *^{L1}—O—CH₂CH₂—**^{L1}, *^{L1}—O—CH₂CH₂CH₂—**^{L1}—CH₂CH₂CH₂CH₂—**^{L1}, *^{L1}—O—CH₂CH₂CH₂CH₂CH₂—**^{L1}, *^{L1}—O—CH₂CH₂CH₂CH₂CH₂CH₂—**^{L1}, *^{L1}—CH₂CH₂CH₂CH₂CH₂CH₂CH₂—**^{L1}, *^{L1}—O—CH₂CH₂CH₂CH₂CH₂CH₂CH₂CH₂—**^{L1}, *^{L1}—CH₂O**^{L1}, *^{L1}—CH₂CH₂—O—**^{L1}, *^{L1}—CH₂CH₂CH₂—O—**^{L1}, *^{L1}—CH₂CH₂CH₂CH₂—O—**^{L1}, *^{L1}—CH₂CH₂CH₂CH₂CH₂—O—**^{L1}, *^{L1}—CH₂CH₂CH₂CH₂CH₂CH₂—O—**^{L1}, *^{L1}—CH₂CH₂CH₂CH₂CH₂CH₂CH₂—O—**^{L1}, *^{L1}—CH₂CH₂CH₂CH₂CH₂CH₂CH₂CH₂—O—**^{L1}, *^{L1}—S—CH₂—**^{L1}, *^{L1}—S—CH₂CH₂—**^{L1}, *^{L1}—S—CH₂CH₂CH₂—**^{L1}, *^{L1}—S—CH₂CH₂CH₂CH₂**^{L1}, *^{L1}—SCH₂CH₂CH₂CH₂CH₂**^{L1}, *^{L1}—SCH₂CH₂CH₂CH₂CH₂CH₂—**^{L1}, *^{L1}—S—CH₂CH₂CH₂CH₂CH₂CH₂CH₂—**^{L1}, *^{L1}—S—CH₂CH₂CH₂CH₂CH₂CH₂CH₂CH₂—**^{L1}, *^{L1}—CH₂—S—**^{L1}, *^{L1}—CH₂CH₂—S—**^{L1}, *^{L1}—CH₂CH₂CH₂—S—**^{L1}, *^{L1}—CH₂CH₂CH₂CH₂—S—**^{L1}, *^{L1}—CH₂CH₂CH₂CH₂CH₂—S—**^{L1}, *^{L1}—CH₂CH₂CH₂CH₂CH₂CH₂—S—**^{L1}, *^{L1}—CH₂CH₂CH₂CH₂CH₂CH₂CH₂—S—**^{L1}, *^{L1}—CH₂CH₂CH₂CH₂CH₂CH₂CH₂CH₂—S—**^{L1}, *^{L1}—O—C₃-C₈cycloalkylene-**^{L1}, *^{L1}—C₃-C₈cycloalkylene-O—**^{L1}, *^{L1}—S—C₃-C₈cycloalkylene-**^{L1}, *^{L1}—C₃-C₈cycloalkylene-S—**^{L1}, *^{L1}—O—CH₂—NR^{L1a}—**^{L1}, *^{L1}—CH₂CH₂—NR^{L1a}—**^{L1}, *^{L0}—CH₂CH₂CH₂—NR^{L1a}—**^{L1}, *^{L1}—O—CH₂CH₂CH₂CH₂—NR^{L1a}—**^{L1}, *^{L1}—O—CH₂CH₂CH₂CH₂CH₂—NR^{L1a}—**^{L1}, *^{L1}—O—CH₂CH₂CH₂CH₂CH₂CH₂—NR^{L1a}—**^{L1}, *^{L1}—O—CH₂CH₂CH₂CH₂CH₂CH₂CH₂—NR^{L1a}—**^{L1}, *^{L1}—O—CH₂CH₂CH₂CH₂CH₂CH₂CH₂CH₂—NR^{L1a}—**^{L1}, *^{L1}—NR^{L1a}—CH₂—O—**^{L1}, *^{L1}—NR^{L1a}—CH₂CH₂—O—**^{L1}, *^{L1}—NR^{L1a}—CH₂CH₂CH₂—O—**^{L1}, *NR^{L1a}—CH₂CH₂CH₂CH₂—O—**^{L1}, *^{L1}—NR^{L1a}—CH₂CH₂CH₂CH₂CH₂—O—**^{L1}, *^{L1}—NR^{L1a}—CH₂CH₂CH₂CH₂CH₂CH₂—O—**^{L1}, *^{L1}—NR^{L1a}—CH₂CH₂CH₂CH₂CH₂CH₂CH—O—**^{L1}, *^{L1}—NR^{L1a}—CH₂CH₂CH₂CH₂CH₂CH₂CH₂CH₂—**^{L1}, *^{L1}—S—CH₂—NR^{L1a}—**^{L1}, *^{L1}—S—CH₂CH₂—NR^{L1a}—**^{L1}, *^{L1}—S—CH₂CH₂CH₂—NR^{L1a}—**^{L1}, *^{L1}—S—CH₂CH₂CH₂CH₂—NR^{L1a}—**^{L1}, *^{L1}—S—CH₂CH₂CH₂CH₂CH₂—NR^{L1a}—**^{L1}, *^{L1}—S—CH₂CH₂CH₂CH₂CH₂CH₂—NR^{L1a}—**^{L1}, *^{L1}—S—CH₂CH₂CH₂CH₂CH₂CH₂CH₂—NR^{L1a}—**^{L1}, *^{L1}—S—CH₂CH₂CH₂CH₂CH₂CH₂CH₂CH₂—NR^{L1a}—**^{L1}, *^{L1}—NR^{L1a}—CH₂—S—**^{L1}, *^{L1}—NR^{L1a}—CH₂CH₂—S—**^{L1}, *^{L1}—NR^{L1a}—CH₂CH₂CH—S—**^{L1}, *^{L1}—NR^{L1a}—CH₂CH₂CH₂CH₂—S—**^{L1}, *^{L1}—NR^{L1a}—CH₂CH₂CH₂CH₂CH₂—S—**^{L1}, *^{L1}—NR^{L1a}—CH₂CH₂CH₂CH₂CH₂CH₂—S—**^{L1}, *^{L1}—NR^{L1a}—CH₂CH₂CH₂CH₂CH₂CH₂CH₂—S—**^{L1}, *NR^{L1a}—CH₂CH₂CH₂CH₂CH₂CH₂CH₂CH₂—S—**^{L1}, *^{L1}—O—CH₂—CO—**^{L1}, *^{L1}—O—CH₂CH₂—CO—**^{L1}, *^{L1}—O—CH₂CH₂CH₂—CO—**^{L1}, *^{L1}—O—CH₂CH₂CH₂CH₂—CO—**^{L1}, *^{L1}—O—CH₂CH₂CH₂CH₂CH₂—CO—**^{L1}, *^{L1}—O—CH₂CH₂CH₂CH₂CH₂CH₂—CO—**^{L1}, *^{L1}—O—CH₂CH₂CH₂CH₂CH₂CH₂CH₂—CO—**^{L1}, *^{L1}—O—CH₂CH₂CH₂CH₂CH₂CH₂CH₂CH₂—CO—**^{L1}, *^{L1}—CO—CH₂—O—**^{L1}—CO—CH₂CH₂—O—**^{L1}, *^{L1}—CO—CH₂CH₂CH₂—O—**^{L1}, *^{L1}—CO—CH₂CH₂CH₂CH₂—O—**^{L1}, *^{L1}—CO—CH₂CH₂CH₂CH₂CH₂—O—**^{L1}, *^{L1}—CO—CH₂CH₂CH₂CH₂CH₂CH₂—O—**^{L1}, *^{L1}—CO—CH₂CH₂CH₂CH₂CH₂CH₂CH₂—O—**^{L1}, *^{L1}—CO—CH₂CH₂CH₂CH₂CH₂CH₂CH₂CH₂—O—**^{L1}, *^{L1}—S—CH₂—CO—**^{L1}, *^{L1}—S—CH₂CH₂—CO—**^{L1}, *^{L1}—S—CH₂CH₂CH₂—CO—**^{L1}, *^{L1}—S—CH₂CH₂CH₂CH₂—CO—**^{L1}, *^{L1}—S—CH₂CH₂CH₂CH₂CH₂—CO—**^{L1}, *^{L1}—S—CH₂CH₂CH₂CH₂CH₂CH₂—CO—**^{L1}, *^{L1}—S—CH₂CH₂CH₂CH₂CH₂CH₂CH₂—CO—**^{L1}, *^{L1}—S—CH₂CH₂CH₂CH₂CH₂CH₂CH₂CH₂—CO—**^{L1}, *^{L1}—CO—CH₂—S—**^{L1}, *^{L1}—CO—CH₂CH₂—S—**^{L1}, *^{L1}—CO—CH₂CH₂CH₂—S—**^{L1}, *^{L1}—CO—CH₂CH₂CH₂CH₂—S—**^{L1}, *^{L1}—CO—CH₂CH₂CH₂CH₂CH₂—S—**^{L1}, *^{L1}—CO—CH₂CH₂CH₂CH₂CH₂CH₂—S—**^{L1}, *^{L1}—CO—CH₂CH₂CH₂CH₂CH₂CH₂CH₂—S—**^{L1}, *^{L1}—CO—CH₂CH₂CH₂CH₂CH₂CH₂CH₂CH₂—S—**^{L1}, *^{L1}—C(O)—CH₂— **^{L1}, *^{L1}—C(O)—CH₂CH₂—**^{L1}, *^{L1}—C(O)—CH₂CH₂CH₂—**^{L1}, *^{L1}—C(O)—CH₂CH₂CH₂CH₂—**^{L1}, *^{L1}—C(O)—CH₂CH₂CH₂CH₂CH₂—**^{L1}, *^{L1}—C(O)—CH₂CH₂CH₂CH₂CH₂CH₂—**^{L1}, *^{L1}—C(O)—CH₂CH₂CH₂CH₂CH₂CH₂CH₂—**^{L1}, *^{L1}—C(O)—CH₂CH₂CH₂CH₂CH₂CH₂CH₂CH₂—**^{L1}, *^{L1}—CH₂—C(O)—**^{L1}, *^{L1}—CH₂CH₂—C(O)—**^{L1}, *^{L1}—CH₂CH₂CH₂—C(O)—**^{L1}, *^{L1}—CH₂CH₂CH₂CH₂—C(O)—**^{L1}, *^{L1}—CH₂CH₂CH₂CH₂CH₂—C(O)—**^{L1}, *^{L1}—CH₂CH₂CH₂CH₂CH₂CH₂—C(O)—**^{L1}, *^{L1}—CH₂CH₂CH₂CH₂CH₂CH₂CH₂—C(O)—**^{L1}, *^{L1}—CH₂CH₂CH₂CH₂CH₂CH₂CH₂CH₂—C(O)—**^{L1}, *^{L1}—CH₂—NR^{L1a}—**^{L1}, *^{L1}—CH₂CH₂—NR^{L1a}—**^{L1}, *^{L1}—CH₂CH₂CH₂—NR^{L1a}—**^{L1}, *^{L1}—CH₂CH₂CH₂CH₂—NR^{L1a}—**^{L1}, *^{L1}—CH₂CH₂CH₂CH₂CH₂—NR^{L1a}—**^{L1}, *^{L1}—CH₂CH₂CH₂CH₂CH₂CH₂—NR^{L1a}—**^{L1}, *^{L1}—CH₂CH₂CH₂CH₂CH₂CH₂CH₂—NR^{L1a}—**^{L1}, *^{L1}—CH₂CH₂CH₂CH₂CH₂CH₂CH₂CH₂—NR^{L1a}—**^{L1}, *^{L1}—NR^{L1a}—CH₂—**^{L1}, *^{L1}—NR^{L1a}—CH₂CH₂—**^{L1}, *^{L1}—NR^{L1a}—CH₂CH₂CH₂—**^{L1}, *^{L1}—NR^{L1a}—CH₂CH₂CH₂CH₂—**^{L1}, *^{L1}—NR^{L1a}—CH₂CH₂CH₂CH₂CH₂—**^{L1}, *^{L1}—NR^{L1a}—CH₂CH₂CH₂CH₂CH₂CH₂—**^{L1}, *^{L1}—NR^{L1a}—CH₂CH₂CH₂CH₂CH₂CH₂CH₂—**^{L1}, *^{L1}—NR^{L1a}—CH₂CH₂CH₂CH₂CH₂CH₂CH₂CH₂—**^{L1}, *^{L1}—CH₂—C(O)NR^{L1a}—**^{L1}, *^{L1}—CH₂CH₂—C(O)NR^{L1a}—**^{L1},*^{L1}—CH₂CH₂CH₂—C(O)NR^{L1a}—**^{L1}, *^{L1}—CH₂CH₂CH₂CH₂—C(O)NR^{L1a}—**^{L1}, *^{L1}—CH₂CH₂CH₂CH₂CH₂—C(O)NR^{L1a}**^{L1}, *^{L1}—CH₂CH₂CH₂CH₂CH₂CH₂—C(O)NR^{L1a}—**^{L1}, *^{L1}—CH₂CH₂CH₂CH₂CH₂CH₂CH₂—C(O)NR^{L1a}—**^{L1}, *^{L1}—CH₂CH₂CH₂CH₂CH₂CH₂CH₂CH₂—C(O)NR^{L1a}—**^{L1}, *^{L1}—C(O)NR^{L1a}—CH₂—**^{L1}, *^{L1}—C(O)NR^{L1a}—CH₂CH₂—**^{L1}, *^{L1}—C(O)NR^{L1a}—CH₂CH₂CH₂—**^{L1}, *^{L1}—C(O)NR^{L1a}—CH₂CH₂CH₂CH₂—**^{L1}, *^{L1}—C(O)NR^{L1a}—CH₂CH₂CH₂CH₂CH₂—**^{L1}, *^{L1}—C(O)NR^{L1a}—CH₂CH₂CH₂CH₂CH₂CH₂—**^{L1}, *^{L1}—C(O)NR^{L1a}—CH₂CH₂CH₂CH₂CH₂CH₂CH₂—**^{L1}, *^{L1}—C(O)NR^{L1a}—CH₂CH₂CH₂CH₂CH₂CH₂CH₂CH₂—**^{L1}, *^{L1}—CH₂—NR^{L1a}C(O)—**^{L1}, *^{L1}—CH₂CH₂—NR^{L1a}C(O)—**^{L1}, *^{L1}—CH₂CH₂CH₂—NR^{L1a} C(O)—**^{L1}, *^{L1}—CH₂CH₂CH₂CH₂—NR^{L1a}C(O)—**^{L1}, *^{L1}—CH₂CH₂CH₂CH₂CH₂—NR^{L1a}C(O)—**^{L1}, *^{L1}—CH₂CH₂CH₂CH₂CH₂CH₂—NR^{L1a}C(O)—**^{L1}, *^{L1}—CH₂CH₂CH₂CH₂CH₂CH₂CH₂—NR^{L1a}C(O)—**^{L1}, *^{L1}—CH₂CH₂CH₂CH₂CH₂CH₂CH₂CH₂—NR^{L1a}C(O)—**^{L1}, *^{L1}—NR^{L1a}C(O)—CH₂—**^{L1}, *^{L1}—NR^{L1a}C(O)—CH₂CH₂—**^{L1}, *^{L1}—NR^{L1a}C(O)—CH₂CH₂CH₂—**^{L1}, *^{L1}—NR^{L1a}C(O)—CH₂CH₂CH₂CH₂—**^{L1}, *^{L1}—NR^{L1a}C(O)—CH₂CH₂CH₂CH₂CH₂—**^{L1}, *^{L1}—NR^{L1a}C(O)—CH₂CH₂CH₂CH₂CH₂CH₂—**^{L1}, *^{L1}—NR^{L1a}C(O)—CH₂CH₂CH₂CH₂CH₂CH₂CH₂—**^{L1}, *^{L1}—NR^{L1a}C(O)—CH₂CH₂CH₂CH₂CH₂CH₂CH₂CH₂—**^{L1}, *^{L1}—C(O)O—CH₂—**^{L1}, *^{L1}—C(O)O—CH₂CH₂—**^{L1}, *^{L1}—C(O)O—CH₂CH₂CH₂—**^{L1}, *^{L1}—C(O)O—CH₂CH₂CH₂CH₂—**^{L1}, *^{L1}—C(O)O—CH₂CH₂CH₂CH₂CH₂—**^{L1}, *^{L1}—C(O)O—CH₂CH₂CH₂CH₂CH₂CH₂—**^{L1}, *^{L1}—C(O)O CH₂CH₂CH₂CH₂CH₂CH₂CH₂—**^{L1}, *^{L1}—C(O)O—CH₂CH₂CH₂CH₂CH₂CH₂CH₂CH₂—**^{L1}, *^{L1}—CH₂—C(O)O—**^{L1}, *^{L1}—CH₂CH₂—C(O)O—**^{L1}, *^{L1}—CH₂CH₂CH₂—C(O)O—**^{L1}, *^{L1}—CH₂CH₂CH₂CH₂—C(O)O—**^{L1}, *^{L1}—CH₂CH₂CH₂CH₂CH₂—C(O)O—**^{L1}, *^{L1}—CH₂CH₂CH₂CH₂CH₂CH₂—C(O)O—**^{L1}, *^{L1}—CH₂CH₂CH₂CH₂CH₂CH₂CH₂—C(O)O—**^{L1}, *^{L1}—CH₂CH₂CH₂CH₂CH₂CH₂CH₂CH₂—C(O)O—**^{L1}, *^{L1}—OC(O)—CH₂—**^{L1}, *^{L1}—OC(O)—CH₂CH₂—**^{L1}, *^{L1}—OC(O)—CH₂CH₂CH₂—**^{L1}, *^{L1}—OC(O)—CH₂CH₂CH₂CH₂—**^{L1}, *^{L1}—OC(O)—CH₂CH₂CH₂CH₂CH₂—**^{L1}, *^{L1}—OC(O)—CH₂CH₂CH₂CH₂CH₂CH₂—**^{L1}, *^{L1}—OC(O)—CH₂CH₂CH₂CH₂CH₂CH₂CH₂—**^{L1}, *^{L1}—OC(O)—CH₂CH₂CH₂CH₂CH₂CH₂CH₂CH₂—**^{L1}, *^{L1}—CH₂—OC(O)—**^{L1}, *^{L1}—CH₂CH₂—OC(O)—**^{L1}, *^{L1}—CH₂CH₂CH₂—OC(O)—**^{L1}, *^{L1}—CH₂CH₂CH₂CH₂—OC(O)—**^{L1}, *^{L1}—CH₂CH₂CH₂CH₂CH₂—OC(O)—**^{L1}, *^{L1}—CH₂CH₂CH₂CH₂CH₂CH₂—OC(O)—**^{L1}, *^{L1}—CH₂CH₂CH₂CH₂CH₂CH₂CH₂—OC(O)—**^{L1}, *^{L1}—CH₂CH₂CH₂CH₂CH₂CH₂CH₂CH₂—OC(O)—**^{L1},

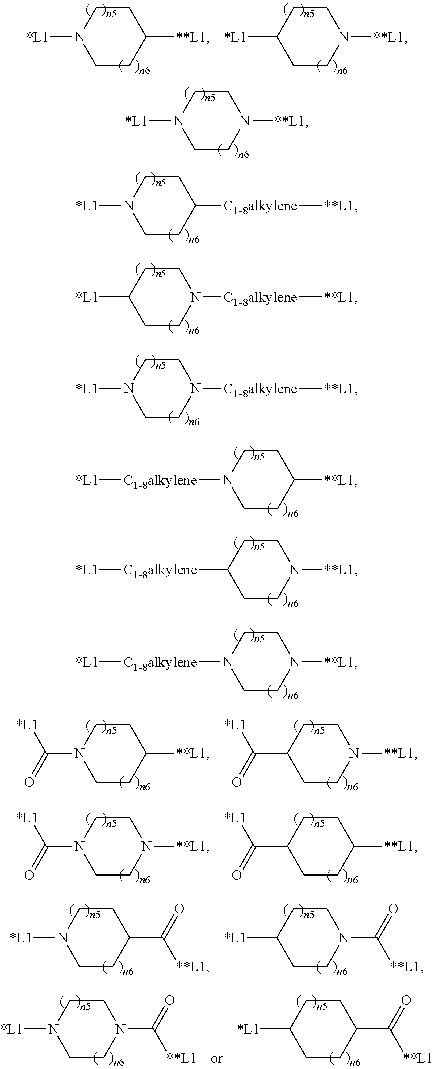

are optionally substituted with at least one R^{L1b};
n5 and n6 are each independently 0 or 1;
R^{L1a} is selected from hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl, each of said —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl is optionally substituted with at least one substituent R^{L1c};
each of said R^{L1b} and R^{L1c} are independently halogen, hydroxy, —C₁₋₈alkyl, —C₁₋₈alkoxy, —C₂₋₈alkenyl, —C₂₋₈alkynyl, C₃-C₈cycloalkyl, 3- to 8-membered heterocyclyl, C₆-C₁₂aryl or 5- to 12-membered heteroaryl; or
two R^{L1b} or two R^{L1c} together with the atoms to which they are attached, form a 3- to 6-membered unsaturated or saturated ring, said ring comprising 0-3 heteroatoms independently selected from nitrogen, oxygen or sulfur; said ring is optionally substituted with at least one substituent halogen, hydroxy, —C₁₋₈alkyl, —C₁₋₈alkoxy, —C₂₋₈alkenyl, —C₂₋₈alkynyl, C₃-C₈cycloalkyl, 3- to 8-membered heterocyclyl, C₆-C₁₂aryl or 5- to 12-membered heteroaryl.

Aspect 17: In some embodiments, wherein L₁ is a single bond,

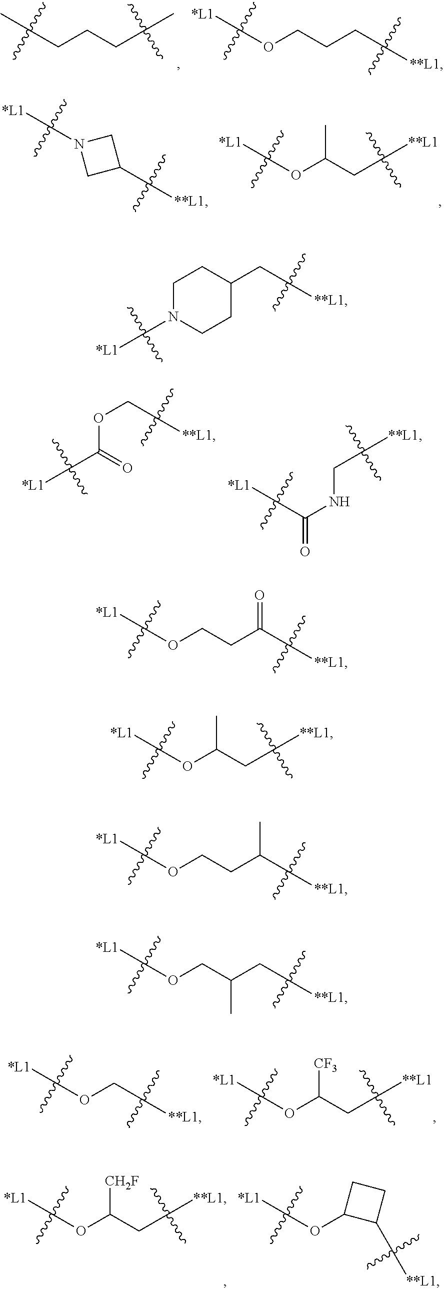

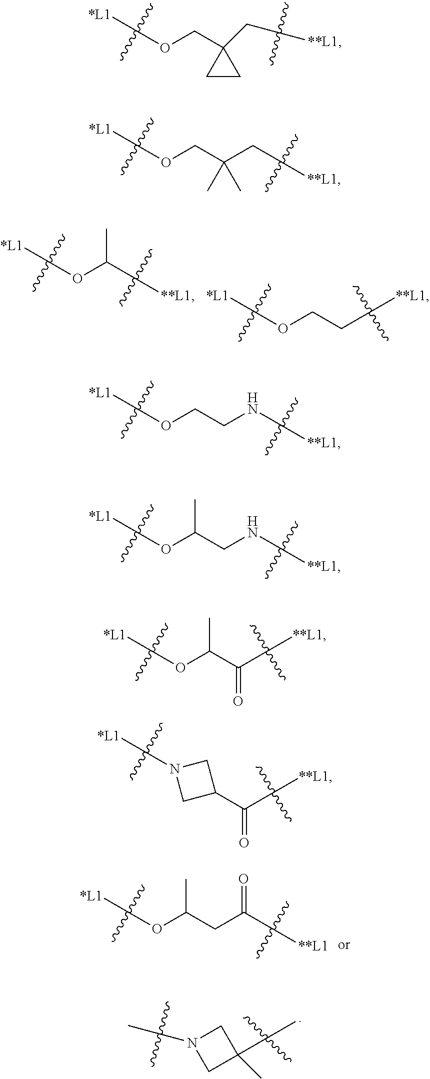

Aspect 18: In some embodiments, wherein R₈ is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 7- to 9-membered spiro-heterocylic ring comprising one or two or three nitrogen atoms as the ring members, oxazolidinyl, imidazolidinyl, thiazolidinyl, pyrazolidinyl, morpholinyl, thiomorpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, phenyl, tetrahydropyridinyl, azetidinyl, pyrrolidinyl, octahydroindolizinyl, octahydroquinolizinyl, hexahydro-1H-pyrrolizinyl, tetrahydroisoquinolinyl, tetrahydropyridyl, oxo, —CN, —OR₈ₐ or —NR₈ₐR_{8b}; wherein each of methyl, ethyl, propyl, butyl, pentyl, hexyl, heptyl, octyl, cyclopropyl, cyclobutyl, cyclopentyl, cyclohexyl, cycloheptyl, cyclooctyl, 7- to 9-membered spiro-heterocylic ring comprising one or two or three nitrogen atoms as the ring members, oxazolidinyl, imidazolidinyl, thiazolidinyl, pyrazolidinyl, morpholinyl, thiomorpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, phenyl or tetrahydropyridinyl optionally substituted with at least one substituent R_{8c};
R₈ₐ and R_{8b} are each independently hydrogen, methyl, ethyl, propyl, butyl, pentyl, hexyl, heptyl, —C₂₋₈alkenyl, —C₂₋₈alkynyl, C₁₋₈alkoxy-C₁₋₈alkyl-, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl or oxo; or
R₈ₐ and R_{8b} together with the carbon atoms to which they are attached, form a 3- to 8-membered unsaturated or saturated ring, said ring comprising 0, 1, 2, or 3 heteroatoms independently selected from nitrogen, oxygen or sulfur; said ring is optionally substituted with at least one substituent R_{8c};
R_{8c}, at each occurrence, is independently halogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, —C₂₋₈alkenyl, —C₂₋₈alkynyl, oxo, —CN, —OR₈ₑ or —NR₈ₑR_{8f}; or
two R_{8c} together with the carbon atoms to which they are attached, form a 3- to 8-membered unsaturated or saturated ring, said ring comprising 0, 1, 2, or 3 heteroatoms independently selected from nitrogen, oxygen or sulfur; said ring is optionally substituted with at least one substituent R_{8d}; or two R_{8c} together with the atoms to which they are attached form a bridge containing one, two, three, or four —CH2-moieties in addition to the two bridgeheads, wherein one of the —CH₂— moiety is optionally replaced with —O—, —S— or —NH—;
R₈ₐ is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, oxo, —CN, —OR_{8g}, —COR_{8g}, —CO₂R_{8g}, —CONR_{8g}R₈ₕ, —NR_{8g}R₈ₕ, —NR_{8g}COR₈ₕ or —NR_{8g}CO₂R₈ₕ, wherein each of methyl, ethyl, propyl, butyl, pentyl, hexyl, heptyl, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl optionally substituted with at least one substituent R₈ᵢ;
R₈ₑ, R_{8f}, R_{8g}, R₈ₕ and R₈ᵢ are each independently hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, —C₂₋₈alkenyl, —C₂₋₈alkynyl, C₁₋₈alkoxy-C₁₋₈alkyl- or C₃-C₈cycloalkyl.

Aspect 19. The compound of any one of Aspects 1-18, wherein R₈ is

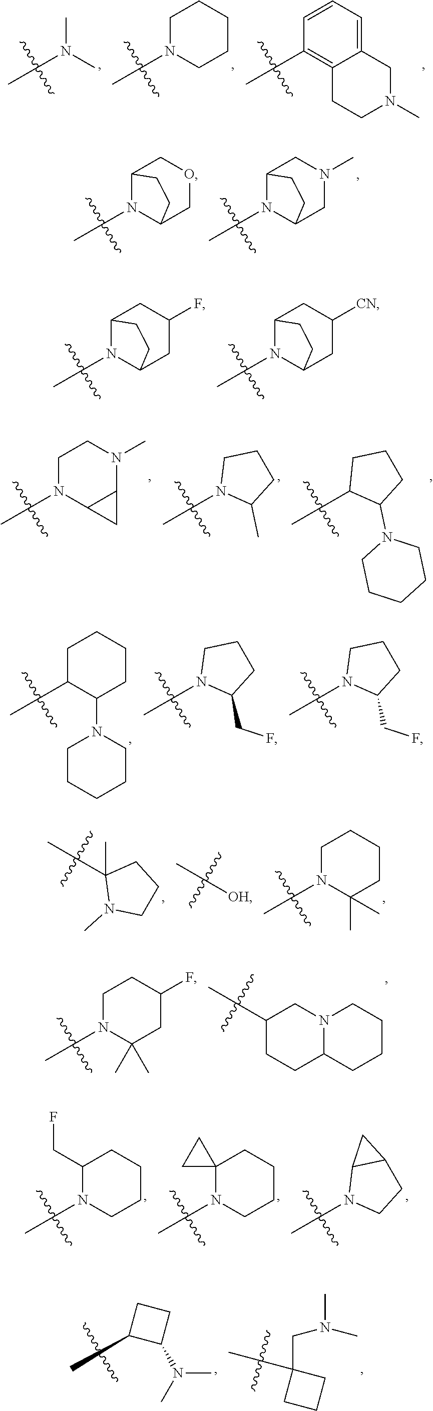

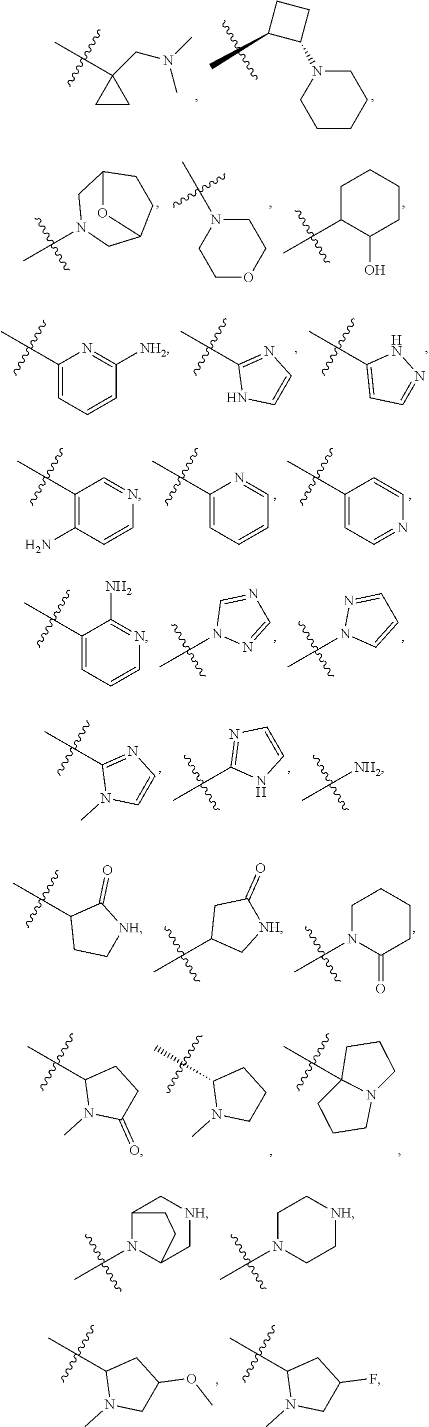

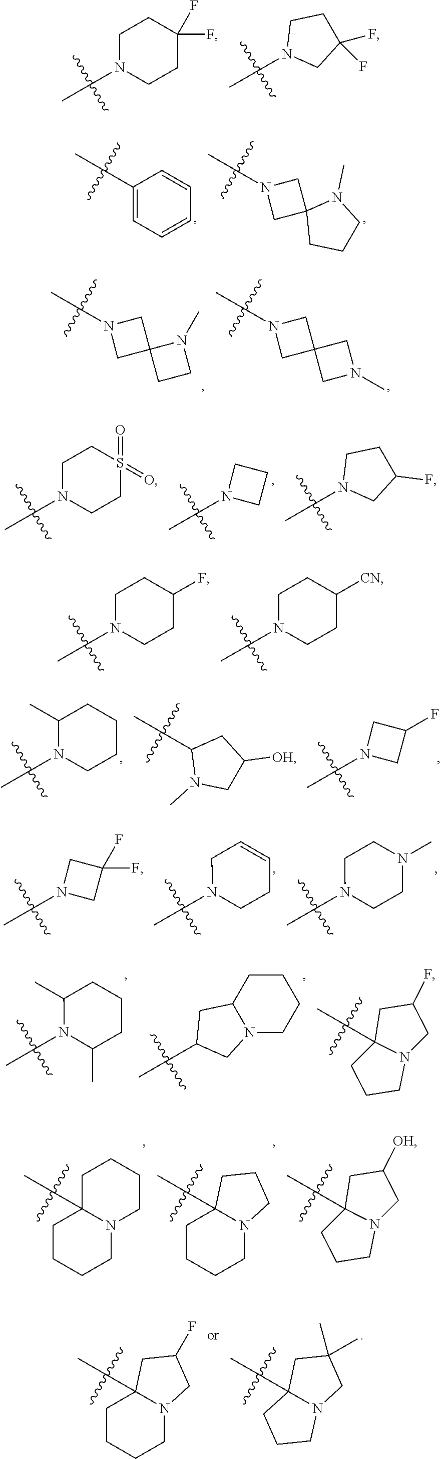

Aspect 19a: In some embodiments, wherein R₈ is

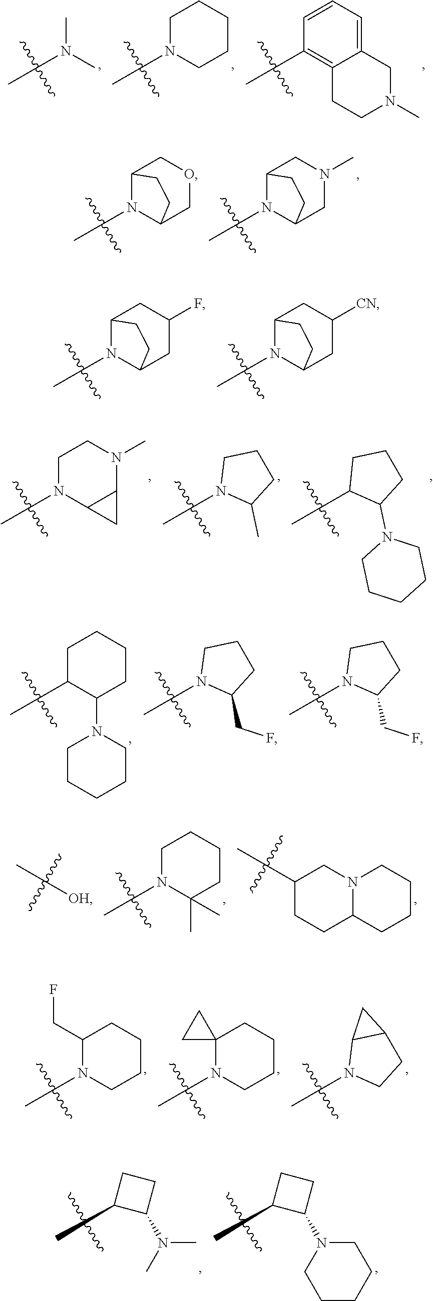

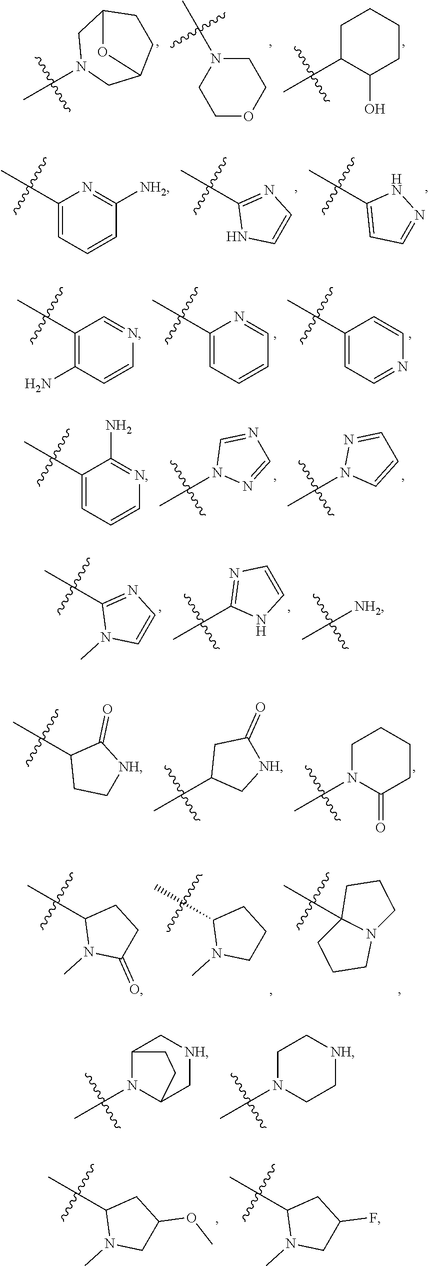

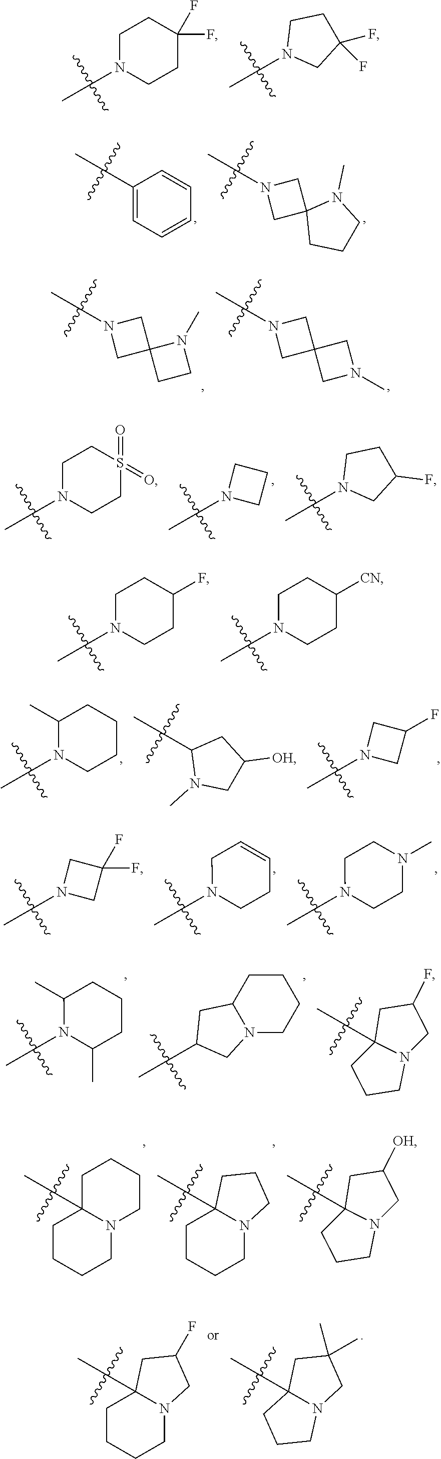

In some embodiments, wherein -L₁-R₈ is

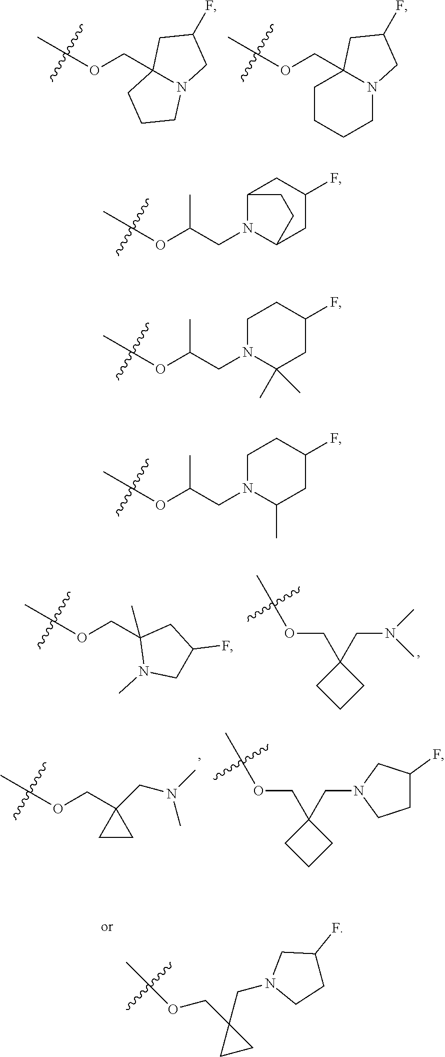

Aspect 19b: The compound of any one of Aspects 1-18, wherein -L₁-R₈ is

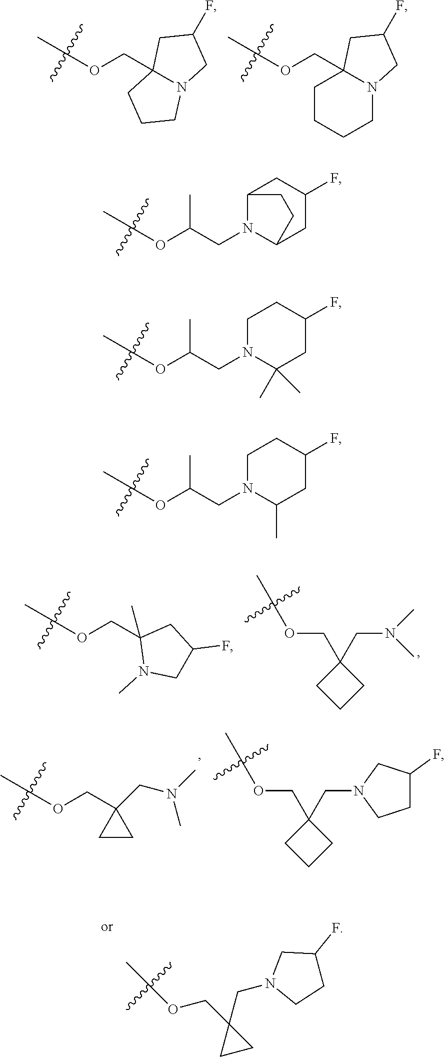

Aspect 20: Provided herein are bridged compounds having the following formula (VIII):

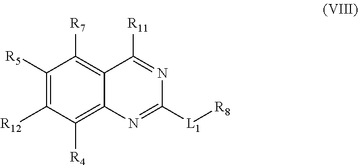

and pharmaceutically acceptable salts, tautomers, isotopologues, stereoisomers and prodrugs thereof, wherein R₄, R₅, R₇, R₈ and L₁ are as defined herein and wherein R₁₁ and R₁₂ are independently substituted or unsubstituted C₁₋₈ alkyl, substituted or unsubstituted cycloalkyl, substituted or unsubstituted aryl, substituted or unsubstituted non-aromatic heterocyclyl, or substituted or unsubstituted heteroaryl.

In one embodiment, when the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

In one embodiment, when the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (—O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

Aspect 20b: Provided herein are bridged compounds having the formula (VIII) and pharmaceutically acceptable salts, tautomers, isotopologues, stereoisomers and prodrugs thereof,
wherein R₄ is methyl, halogen;
R₅ is H, halogen, substituted or unsubstituted C₁₋₈alkyl, substituted or unsubstituted C₃₋₁₀cycloalkyl, substituted or unsubstituted C₂₋₁₀alkenyl, halogen, —CF₃, —O—CF₃ or —S—CF₃;
R₇ is hydrogen;
R₈ is substituted or unsubstituted C₃₋₁₀cycloalkyl, substituted or unsubstituted C₆₋₁₀aryl substituted or unsubstituted heterocyclyl, substituted or unsubstituted heteroaryl, —NH₂, substituted or unsubstituted —NHC₁₋₈alkyl, substituted or unsubstituted —N(C₁₋₈alkyl)₂, aminocarbonyl, hydroxyl, substituted or unsubstituted —NH-heteroaryl, —CF₃ or hydroxyl;
L₁ is —O—, substituted or unsubstituted —OC₁₋₈alkyl, substituted or unsubstituted heterocyclyl, or substituted or unsubstituted heteroaryl;
R₁₁ is substituted or unsubstituted C₃₋₁₀cycloalkyl, substituted or unsubstituted C₆₋₁₀aryl substituted or unsubstituted heterocyclyl, or substituted or unsubstituted heteroaryl; and
R₁₂ is substituted or unsubstituted C₃₋₁₀cycloalkyl, substituted or unsubstituted C₆₋₁₀aryl substituted or unsubstituted heterocyclyl, or substituted or unsubstituted heteroaryl.

Aspect 20c: Provided herein are bridged compounds having the formula (VIII) and pharmaceutically acceptable salts, tautomers, isotopologues, stereoisomers and prodrugs thereof,
wherein R₄ is methyl, halogen;
R₅ is H, halogen, substituted or unsubstituted C₁₋₈alkyl, substituted or unsubstituted C₃₋₁₀cycloalkyl, substituted or unsubstituted C₂₋₁₀alkenyl, halogen, —CF₃, —O—CF₃ or —S—CF₃;
R₇ is hydrogen;
R₁₁ is substituted or unsubstituted C₃₋₁₀cycloalkyl, substituted or unsubstituted C₆₋₁₀aryl substituted or unsubstituted heterocyclyl, or substituted or unsubstituted heteroaryl;
R₁₂ is substituted or unsubstituted C₃₋₁₀cycloalkyl, substituted or unsubstituted C₆₋₁₀aryl substituted or unsubstituted heterocyclyl, or substituted or unsubstituted heteroaryl; and
-L₁-R₈ is

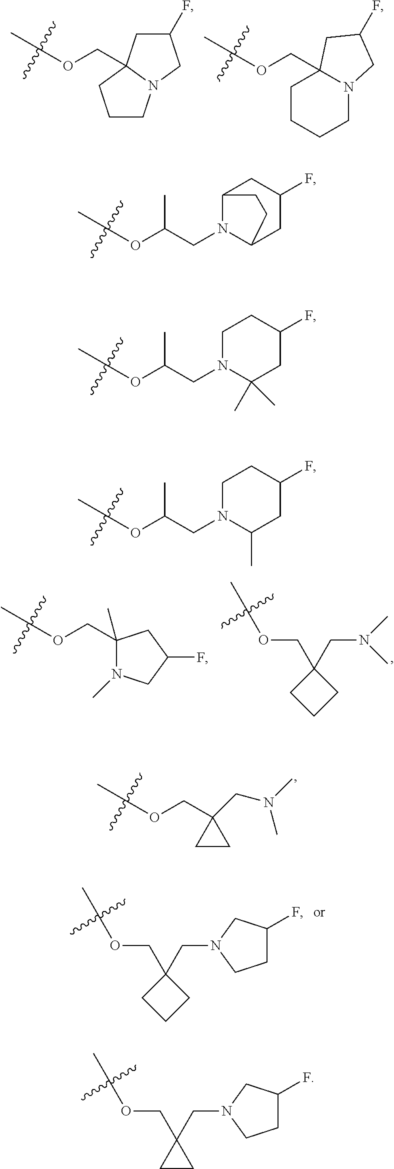

Aspect 20d: Provided herein are bridged compounds having the formula (VIII) and pharmaceutically acceptable salts, tautomers, isotopologues, stereoisomers and prodrugs thereof,
wherein R₄ is methyl, halogen;
R₅ is H, halogen, substituted or unsubstituted C₁₋₈alkyl, substituted or unsubstituted C₃₋₁₀cycloalkyl, substituted or unsubstituted C₂₋₁₀alkenyl, halogen, —CF₃, —O—CF₃, or —S—CF₃;
R₇ is hydrogen;
R₈ is substituted or unsubstituted C₃₋₁₀cycloalkyl, substituted or unsubstituted C₆₋₁₀aryl substituted or unsubstituted heterocyclyl, substituted or unsubstituted heteroaryl, —NH₂, substituted or unsubstituted —NHC₁₋₈alkyl, substituted or unsubstituted —N(C₁₋₈alkyl)₂, aminocarbonyl, hydroxyl, substituted or unsubstituted —NH-heteroaryl, —CF₃ or hydroxyl;
L₁ is —O—, substituted or unsubstituted —OC₁₋₈alkyl, substituted or unsubstituted heterocyclyl, or substituted or unsubstituted heteroaryl;
R₁₁ is substituted or unsubstituted C₃₋₁₀cycloalkyl, substituted or unsubstituted C₆₋₁₀aryl substituted or unsubstituted heterocyclyl or substituted or unsubstituted heteroaryl; and
R₁₂ is

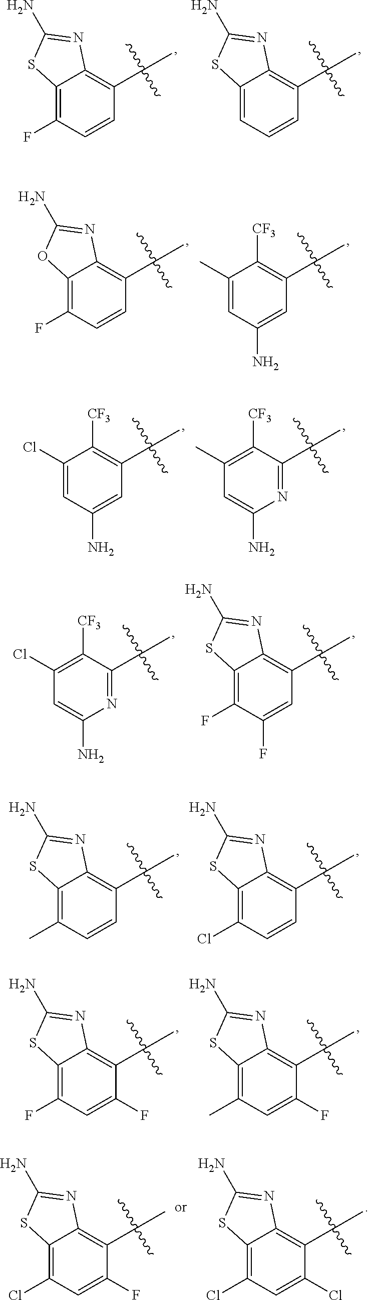

Aspect 20e: Provided herein are bridged compounds having the formula (VIII) and pharmaceutically acceptable salts, tautomers, isotopologues, stereoisomers and prodrugs thereof,
wherein R₄ is methyl, halogen;
R₅ is substituted or unsubstituted C₁₋₈alkyl, substituted or unsubstituted C₃₋₁₀cycloalkyl, substituted or unsubstituted C₂₋₁₀alkenyl, halogen, —CF₃, or —S—CF₃;
R₇ is hydrogen;
R₈ is substituted or unsubstituted C₃₋₁₀cycloalkyl, substituted or unsubstituted C₆₋₁₀aryl substituted or unsubstituted heterocyclyl, substituted or unsubstituted heteroaryl, —NH₂, substituted or unsubstituted —NHC₁₋₈alkyl, substituted or unsubstituted —N(C₁₋₈alkyl)₂, aminocarbonyl, hydroxyl, substituted or unsubstituted —NH-heteroaryl, —CF₃ or hydroxyl;
L₁ is —O—, substituted or unsubstituted —OC₁₋₈alkyl, substituted or unsubstituted heterocyclyl, or substituted or unsubstituted heteroaryl;
R₁₁ is a substituted or unsubstituted bridged bicyclic ring; and
R₁₂ is substituted or unsubstituted C₃₋₁₀cycloalkyl, substituted or unsubstituted C₆₋₁₀aryl substituted or unsubstituted heterocyclyl, or substituted or unsubstituted heteroaryl.

Aspect 20f: Provided herein are bridged compounds having the formula (VIII) and pharmaceutically acceptable salts, tautomers, isotopologues, stereoisomers and prodrugs thereof,
wherein R₄ is methyl, halogen;
R₅ is substituted or unsubstituted C₁₋₈alkyl, substituted or unsubstituted C₃₋₁₀cycloalkyl, substituted or unsubstituted C₂₋₁₀alkenyl, halogen, —CF₃, or —S—CF₃;
R₇ is hydrogen;
R₈ is substituted or unsubstituted C₃₋₁₀cycloalkyl, substituted or unsubstituted C₆₋₁₀aryl substituted or unsubstituted heterocyclyl, substituted or unsubstituted heteroaryl, —NH₂, substituted or unsubstituted —NHC₁₋₈alkyl, substituted or unsubstituted —N(C₁₋₈alkyl)₂, aminocarbonyl, hydroxyl, substituted or unsubstituted —NH-heteroaryl, —CF₃ or hydroxyl;
L₁ is —O—, substituted or unsubstituted —OC₁₋₈alkyl, substituted or unsubstituted heterocyclyl, or substituted or unsubstituted heteroaryl;
R₁₁ is a substituted or unsubstituted bridged bicyclic ring, wherein R₁₁ does not contain an acryloyl group or substituted acryloyl group; and
R₁₂ is substituted or unsubstituted C₆₋₁₀aryl, substituted or unsubstituted 5- to 7-membered monocyclic heteroaryl, or substituted or unsubstituted 8- to 12-membered bicyclic heterocyclyl.

Aspect 21: Provided herein are bridged compounds having the formula (VIII) and pharmaceutically acceptable salts, tautomers, isotopologues, stereoisomers and prodrugs thereof,
wherein R₄ is methyl, halogen;
R₅ is substituted or unsubstituted C₁₋₈alkyl, substituted or unsubstituted C₃₋₁₀cycloalkyl, substituted or unsubstituted C₂₋₁₀alkenyl, halogen, CF₃, or —S—CF₃;
R₇ is hydrogen;
R₈ is substituted or unsubstituted C₃₋₁₀cycloalkyl, substituted or unsubstituted heterocyclyl, substituted or unsubstituted heteroaryl, —NH₂, substituted or unsubstituted —NHC₁₋₈alkyl, substituted or unsubstituted —N(C₁₋₈alkyl)₂, aminocarbonyl, hydroxyl, substituted or unsubstituted —NH-heteroaryl, —CF₃ or hydroxyl;
L₁ is —O—, substituted or unsubstituted —OC₁₋₈alkyl, substituted or unsubstituted heterocyclyl, or substituted or unsubstituted heteroaryl;
R₁₁ is substituted or unsubstituted heterocyclyl; and
R₁₂ is substituted or unsubstituted aryl, or substituted or unsubstituted heteroaryl.

Aspect 21b: Provided herein are bridged compounds having the formula (VIII) and pharmaceutically acceptable salts, tautomers, isotopologues, stereoisomers and prodrugs thereof,
wherein R₄ is methyl, halogen;
R₅ is substituted or unsubstituted C₁₋₈alkyl, substituted or unsubstituted C₃₋₁₀cycloalkyl, substituted or unsubstituted C₂₋₁₀alkenyl, halogen, CF₃, or —S—CF₃;
R₇ is hydrogen;
R₈ is substituted or unsubstituted C₃₋₁₀cycloalkyl, substituted or unsubstituted heterocyclyl, substituted or unsubstituted heteroaryl, —NH₂, substituted or unsubstituted —NHC₁₋₈alkyl, substituted or unsubstituted —N(C₁₋₈alkyl)₂, aminocarbonyl, hydroxyl, substituted or unsubstituted —NH-heteroaryl, —CF₃ or hydroxyl;
L₁ is —O—, substituted or unsubstituted —OC₁₋₈alkyl, substituted or unsubstituted heterocyclyl, or substituted or unsubstituted heteroaryl;
R₁₁ is a substituted or unsubstituted bridged bicyclic ring; and
R₁₂ is substituted or unsubstituted aryl, or substituted or unsubstituted heteroaryl.

Aspect 22: Provided herein are bridged compounds having the formula (VIII) and pharmaceutically acceptable salts, tautomers, isotopologues, stereoisomers and prodrugs thereof,
wherein R₄ is halogen;
R₅ is C₁₋₈alkyl, C₃₋₁₀cycloalkyl, C₂₋₁₀alkenyl, halogen, CF₃, or —S—CF₃, wherein the C₁₋₈alkyl, C₃₋₁₀cycloalkyl, or C₂₋₁₀alkenyl is optionally substituted with one, two or three halogen;
R₇ is hydrogen;
R₈ is C₃₋₁₀cycloalkyl, heterocyclyl, heteroaryl, —NH₂, —NHC₁₋₈alkyl, —N(C₁₋₈alkyl)₂, aminocarbonyl, hydroxyl, —NH-heteroaryl, —CF₃ or hydroxyl, optionally substituted with halogen, nitril, hydroxyl, C₁₋₈alkyl, SO₂, —OC₁₋₈alkyl, heterocyclyl, —CH₂F, C₃₋₁₀cycloalkyl, —NH₂, —NHC₁₋₈alkyl, —N(C₁₋₈alkyl)₂, C₂₋₁₀alkenyl or oxo (═O);
L₁ is —O—, —OC₁₋₈alkyl, heterocyclyl, or heteroaryl, optionally substituted with oxo (═O), —CF₃, —CH₂F, or C₃₋₁₀cycloalkyl;
R₁₁ is heterocyclyl, optionally substituted with —NH₂, —NHR_{Y1b}, or —N(R_{Y1b})₂, where R_{Y1b} is C₁₋₈alkyl optionally substituted with one, two or three halogen or R_{Y1b} is C₁₋₈alkyl optionally substituted with one —OCH₃; and
R₁₂ is aryl, or heteroaryl, optionally substituted with hydroxyl, amino, halogen, C₁₋₈alkyl, CF₃, heteroaryl, C₃₋₁₀cycloalkyl, —NH₂, —NHC₁₋₈alkyl, —N(C₁₋₈alkyl)₂, or C₂₋₁₀alkenyl.

Aspect 22b: Provided herein are bridged compounds having the formula (VIII) and pharmaceutically acceptable salts, tautomers, isotopologues, stereoisomers and prodrugs thereof,
wherein R₄ is halogen;
R₅ is C₁₋₈alkyl, C₃₋₁₀cycloalkyl, C₂₋₁₀alkenyl, halogen, CF₃, or —S—CF₃, optionally substituted with one, two or three halogen;
R₇ is hydrogen;
R₈ is C₃₋₁₀cycloalkyl, heterocyclyl, heteroaryl, —NH₂, —NHC₁₋₈alkyl, —N(C₁₋₈alkyl)₂, aminocarbonyl, hydroxyl, —NH-heteroaryl, —CF₃ or hydroxyl, optionally substituted with halogen, nitril, hydroxyl, C₁₋₈alkyl, SO₂, —OC₁₋₈alkyl, heterocyclyl, —CH₂F, C₃₋₁₀cycloalkyl, —NH₂, —NHC₁₋₈alkyl, —N(C₁₋₈alkyl)₂, C₂₋₁₀alkenyl or oxo (═O);
L₁ is —O—, —OC₁₋₈alkyl, heterocyclyl, or heteroaryl, optionally substituted with oxo (═O), —CF₃, —CH₂F, or C₃₋₁₀cycloalkyl;
R₁₁ is a bridged bicyclic ring, optionally substituted with —NH₂, —NHR_{Y1b}, or —N(R_{Y1b})₂, where R_{Y1b} is C₁₋₈alkyl optionally substituted with one, two or three halogen or R_{Y1b} is C₁₋₈alkyl optionally substituted with one —OCH₃; and
R₁₂ is aryl, or heteroaryl, optionally substituted with hydroxyl, amino, halogen, C₁₋₈alkyl, CF₃, heteroaryl, C₃₋₁₀cycloalkyl, —NH₂, —NHC₁₋₈alkyl, —N(C₁₋₈alkyl)₂, or C₂₋₁₀alkenyl.

Aspect 23: Provided herein are bridged compounds having the formula (VIII) and pharmaceutically acceptable salts, tautomers, isotopologues, stereoisomers and prodrugs thereof,
wherein R₄ is chloro, fluoro;
R₅ is C₁₋₈alkyl, C₃₋₁₀cycloalkyl, C₂₋₁₀alkenyl, chloro, CF₃, or —S—CF₃, optionally substituted with one, two or three fluoro;
R₇ is hydrogen;
R₈ is C₃₋₁₀cycloalkyl, heterocyclyl, heteroaryl, —NH₂, —NHC₁₋₈alkyl, —N(C₁₋₈alkyl)₂, aminocarbonyl, hydroxyl, —NH-heteroaryl, —CF₃ or hydroxyl, optionally substituted with fluoro, nitril, hydroxyl, C₁₋₈alkyl, SO₂, —OC₁₋₈alkyl, heterocyclyl, —CH₂F, C₃₋₁₀cycloalkyl, —NH₂, —NHC₁₋₈alkyl, —N(C₁₋₈alkyl)₂, C₂₋₁₀alkenyl or oxo (═O);
L₁ is —O—, —OC₁₋₈alkyl, heterocyclyl, or heteroaryl, optionally substituted with oxo (═O), —CF₃, —CH₂F, or C₃₋₁₀cycloalkyl;
R₁₁ is heterocyclyl, optionally substituted with —NH₂, —NHR_{Y1b}, or —N(R_{Y1b})₂, where R_{Y1b} is C₁₋₈alkyl optionally substituted with one, two or three fluoro or R_{Y1b} is C₁₋₈alkyl optionally substituted with one —OCH₃; and
R₁₂ is aryl, or heteroaryl, optionally substituted with hydroxyl, amino, halogen, C₁₋₈alkyl, CF₃, heteroaryl, C₃₋₁₀cycloalkyl, —NH₂, —NHC₁₋₈alkyl, —N(C₁₋₈alkyl)₂, or C₂₋₁₀alkenyl.

Aspect 23b: Provided herein are bridged compounds having the formula (VIII) and pharmaceutically acceptable salts, tautomers, isotopologues, stereoisomers and prodrugs thereof,
wherein R₄ is chloro, fluoro;
R₅ is C₁₋₈alkyl, C₃₋₁₀cycloalkyl, C₂₋₁₀alkenyl, chloro, CF₃, or —S—CF₃, optionally substituted with one, two or three fluoro;
R₇ is hydrogen;
R₈ is C₃₋₁₀cycloalkyl, heterocyclyl, heteroaryl, —NH₂, —NHC₁₋₈alkyl, —N(C₁₋₈alkyl)₂, aminocarbonyl, hydroxyl, —NH-heteroaryl, —CF₃ or hydroxyl, optionally substituted with fluoro, nitril, hydroxyl, C₁₋₈alkyl, SO₂, —OC₁₋₈alkyl, heterocyclyl, —CH₂F, C₃₋₁₀cycloalkyl, —NH₂, —NHC₁₋₈alkyl, —N(C₁₋₈alkyl)₂, C₂₋₁₀alkenyl or oxo (═O);
L₁ is —O—, —OC₁₋₈alkyl, heterocyclyl, or heteroaryl, optionally substituted with oxo (═O), —CF₃, —CH₂F, or C₃₋₁₀cycloalkyl;
R₁₁ is a bridged bicyclic ring, optionally substituted with —NH₂, —NHR_{Y1b}, or —N(R_{Y1b})₂, where R_{Y1b} is C₁₋₈alkyl optionally substituted with one, two or three fluoro or R_{Y1b} is C₁₋₈alkyl optionally substituted with one —OCH₃; and
R₁₂ is aryl, or heteroaryl, optionally substituted with hydroxyl, amino, halogen, C₁₋₈alkyl, CF₃, heteroaryl, C₃₋₁₀cycloalkyl, —NH₂, —NHC₁₋₈alkyl, —N(C₁₋₈alkyl)₂, or C₂₋₁₀alkenyl.

Aspect 24: Provided herein are bridged compounds having the formula (VIII) and pharmaceutically acceptable salts, tautomers, isotopologues, stereoisomers and prodrugs thereof,
wherein R₄ is halogen;
R₅ is —CF₃, —CF₂CH₃;
R₇ is hydrogen;
R₈ is C₃₋₁₀cycloalkyl, heterocyclyl, heteroaryl, —NH₂, —NHC₁₋₈alkyl, —N(C₁₋₈alkyl)₂, aminocarbonyl, hydroxyl, —NH-heteroaryl, —CF₃ or hydroxyl, optionally substituted with halogen, nitril, hydroxyl, C₁₋₈alkyl, SO₂, —OC₁₋₈alkyl, heterocyclyl, —CH₂F, C₃₋₁₀cycloalkyl, —NH₂, —NHC₁₋₈alkyl, —N(C₁₋₈alkyl)₂, C₂₋₁₀alkenyl or oxo (═O);
L₁ is —O—, —OC₁₋₈alkyl, heterocyclyl, or heteroaryl, optionally substituted with oxo (═O), —CF₃, —CH₂F, or C₃₋₁₀cycloalkyl;
R₁₁ is heterocyclyl, optionally substituted with —NH₂, —NHR_{Y1b}, or —N(R_{Y1b})₂, where R_{Y1b} is C₁₋₈alkyl optionally substituted with one, two or three halogen or R_{Y1b} is C₁₋₈alkyl optionally substituted with one —OCH₃; and
R₁₂ is aryl, or heteroaryl, optionally substituted with hydroxyl, amino, halogen, C₁₋₈alkyl, CF₃, heteroaryl, C₃₋₁₀cycloalkyl, —NH₂, —NHC₁₋₈alkyl, —N(C₁₋₈alkyl)₂, or C₂₋₁₀alkenyl.

Aspect 24b: Provided herein are bridged compounds having the formula (VIII) and pharmaceutically acceptable salts, tautomers, isotopologues, stereoisomers and prodrugs thereof,
wherein R₄ is halogen;
R₅ is —CF₃, —CF₂CH₃;
R₇ is hydrogen;
R₈ is C₃₋₁₀cycloalkyl, heterocyclyl, heteroaryl, —NH₂, —NHC₁₋₈alkyl, —N(C₁₋₈alkyl)₂, aminocarbonyl, hydroxyl, —NH-heteroaryl, —CF₃ or hydroxyl, optionally substituted with halogen, nitril, hydroxyl, C₁₋₈alkyl, SO₂, —OC₁₋₈alkyl, heterocyclyl, —CH₂F, C₃₋₁₀cycloalkyl, —NH₂, —NHC₁₋₈alkyl, —N(C₁₋₈alkyl)₂, C₂₋₁₀alkenyl or oxo (═O);
L₁ is —O—, —OC₁₋₈alkyl, heterocyclyl, or heteroaryl, optionally substituted with oxo (═O), —CF₃, —CH₂F, or C₃₋₁₀cycloalkyl;
R₁₁ is a bridged bicyclic ring, optionally substituted with —NH₂, —NHR_{Y1b}, or —N(R_{Y1b})₂, where R_{Y1b} is C₁₋₈alkyl optionally substituted with one, two or three halogen or R_{Y1b} is C₁₋₈alkyl optionally substituted with one —OCH₃; and
R₁₂ is aryl, or heteroaryl, optionally substituted with hydroxyl, amino, halogen, C₁₋₈alkyl, CF₃, heteroaryl, C₃₋₁₀cycloalkyl, —NH₂, —NHC₁₋₈alkyl, —N(C₁₋₈alkyl)₂, or C₂₋₁₀alkenyl.

Aspect 25a: Provided herein are bridged compounds having the formula (VIII) and pharmaceutically acceptable salts, tautomers, isotopologues, stereoisomers and prodrugs thereof,
wherein R₄ is methyl, halogen;
R₅ is C₁₋₈alkyl, C₃₋₁₀cycloalkyl, C₂₋₁₀alkenyl, halogen, CF₃, or —S—CF₃, optionally substituted with one, two or three halogen;
R₇ is hydrogen;
R₈ is C₃₋₁₀cycloalkyl, heterocyclyl, heteroaryl, —NH₂, —NHC₁₋₈alkyl, —N(C₁₋₈alkyl)₂, aminocarbonyl, hydroxyl, —NH-heteroaryl, —CF₃ or hydroxyl, optionally substituted with halogen, nitril, hydroxyl, C₁₋₈alkyl, SO₂, —OC₁₋₈alkyl, heterocyclyl, —CH₂F, C₃₋₁₀cycloalkyl, —NH₂, —NHC₁₋₈alkyl, —N(C₁₋₈alkyl)₂, C₂₋₁₀alkenyl or oxo (═O);
L₁ is —O—, —OC₁₋₈alkyl, heterocyclyl, or heteroaryl, optionally substituted with oxo (═O), —CF₃, —CH₂F, or C₃₋₁₀cycloalkyl;
R₁₁ is -3,8-diazabicyclo[3.2.1]octane,

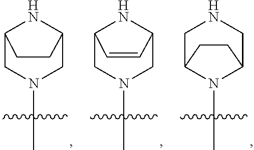

3-oxa-7,9-diazabicyclo[3.3.1]nonane,

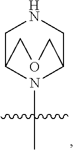 [SMILES: *N1C2CNCC1COC2]

3,6-diazabicyclo[3.1.1]heptane,

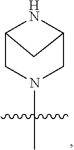 [SMILES: *N1CC2CC(C1)N2]

2,5-diazabicyclo[2.2.2]octane,

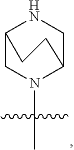 [SMILES: *N1CC2CCC1CN2]

2,5-diazabicyclo[2.2.1]heptane,

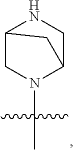 [SMILES: *N1CC2CC1CN2]

7-azabicyclo[2.2.1]heptan-7-yl,

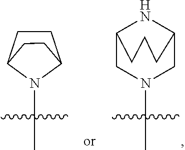 [SMILES: *N1C2CCC1CC2 ; *N1CC2CCCC(C1)N2]

optionally substituted with —NH₂, —NHR_{Y1b}, or —N(R_{Y1b})₂, where R_{Y1b} is C₁₋₈alkyl optionally substituted with one, two or three halogen or R_{Y1b} is C₁₋₈alkyl optionally substituted with one —OCH₃; and
R₁₂ is aryl, or heteroaryl, optionally substituted with hydroxyl, amino, halogen, C₁₋₈alkyl, CF₃, heteroaryl, C₃₋₁₀cycloalkyl, —NH₂, —NHC₁₋₈alkyl, —N(C₁₋₈alkyl)₂, or C₂₋₁₀alkenyl.

Aspect 25: Provided herein are bridged compounds having the formula (VIII) and pharmaceutically acceptable salts, tautomers, isotopologues, stereoisomers and prodrugs thereof,
wherein R₄ is methyl, halogen;
R₅ is C₁₋₈alkyl, C₃₋₁₀cycloalkyl, C₂₋₁₀alkenyl, halogen, CF₃, or —S—CF₃, optionally substituted with one, two or three halogen;
R₇ is hydrogen;
R₈ is C₃₋₁₀cycloalkyl, heterocyclyl, heteroaryl, —NH₂, —NHC₁₋₈alkyl, —N(C₁₋₈alkyl)₂, aminocarbonyl, hydroxyl, —NH-heteroaryl, —CF₃ or hydroxyl, optionally substituted with halogen, nitril, hydroxyl, C₁₋₈alkyl, SO₂, —OC₁₋₈alkyl, heterocyclyl, —CH₂F, C₃₋₁₀cycloalkyl, —NH₂, —NHC₁₋₈alkyl, —N(C₁₋₈alkyl)₂, C₂₋₁₀alkenyl or oxo (═O);
L₁ is —O—, —OC₁₋₈alkyl, heterocyclyl, or heteroaryl, optionally substituted with oxo (═O), —CF₃, —CH₂F, or C₃₋₁₀cycloalkyl;
R₁₁ is 3-oxa-7,9-diazabicyclo[3.3.1]nonane,

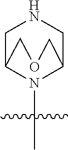 [SMILES: *N1C2CNCC1COC2]

3,6-diazabicyclo[3.1.1]heptane,

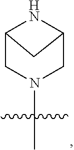 [SMILES: *N1CC2CC(C1)N2]

2,5-diazabicyclo[2.2.2]octane,

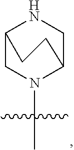 [SMILES: *N1CC2CCC1CN2]

2,5-diazabicyclo[2.2.1]heptane,

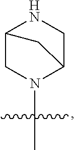 [SMILES: *N1CC2CC1CN2]

7-azabicyclo[2.2.1]heptan-7-yl,

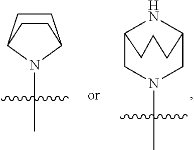 [SMILES: *N1C2CCC1CC2 ; *N1CC2CCCC(C1)N2]

optionally substituted with —NH₂, —NHR_{Y1b}, or —N(R_{Y1b})₂, where R_{Y1b} is C₁₋₈alkyl optionally substituted with one, two or three halogen or R_{Y1b} is C₁₋₈alkyl optionally substituted with one —OCH₃; and
R₁₂ is aryl, or heteroaryl, optionally substituted with hydroxyl, amino, halogen, C₁₋₈alkyl, CF₃, heteroaryl, C₃₋₁₀cycloalkyl, —NH₂, —NHC₁₋₈alkyl, —N(C₁₋₈alkyl)₂, or C₂₋₁₀alkenyl.

Aspect 25b: Provided herein are bridged compounds having the formula (VIII) and pharmaceutically acceptable salts, tautomers, isotopologues, stereoisomers and prodrugs thereof,
wherein R₄ is methyl, halogen;
R₅ is C₁₋₈alkyl, C₃₋₁₀cycloalkyl, C₂₋₁₀alkenyl, halogen, CF₃, or —S—CF₃, optionally substituted with one, two or three halogen;
R₇ is hydrogen;
R₈ is C₃₋₁₀cycloalkyl, heterocyclyl, heteroaryl, —NH₂, —NHC₁₋₈alkyl, —N(C₁₋₈alkyl)₂, aminocarbonyl, hydroxyl, —NH-heteroaryl, —CF₃ or hydroxyl, optionally substituted with halogen, nitril, hydroxyl, C₁₋₈alkyl, SO₂, —OC₁₋₈alkyl, heterocyclyl, —CH₂F, C₃₋₁₀cycloalkyl, —NH₂, —NHC₁₋₈alkyl, —N(C₁₋₈alkyl)₂, C₂₋₁₀alkenyl or oxo (═O);
L₁ is —O—, —OC₁₋₈alkyl, heterocyclyl, or heteroaryl, optionally substituted with oxo (═O), —CF₃, —CH₂F, or C₃₋₁₀cycloalkyl;
R₁₁ is -3,8-diazabicyclo[3.2.1]octane,

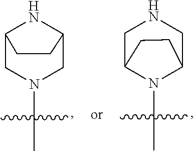 [SMILES: *N1CC2CCC(C1)N2 ; *N1C2CCC1CNC2]

and
R₁₂ is aryl, or heteroaryl, optionally substituted with hydroxyl, amino, halogen, C₁₋₈alkyl, CF₃, heteroaryl, C₃₋₁₀cycloalkyl, —NH₂, —NHC₁₋₈alkyl, —N(C₁₋₈alkyl)₂, or C₂₋₁₀alkenyl.

Aspect 25c: Provided herein are bridged compounds having the formula (VIII) and pharmaceutically acceptable salts, tautomers, isotopologues, stereoisomers and prodrugs thereof,
wherein R₄ is methyl, halogen;
R₅ is C₁₋₈alkyl, C₃₋₁₀cycloalkyl, C₂₋₁₀alkenyl, halogen, CF₃, or —S—CF₃, optionally substituted with one, two or three halogen;
R₇ is hydrogen;
R₈ is C₃₋₁₀cycloalkyl, heterocyclyl, heteroaryl, —NH₂, —NHC₁₋₈alkyl, —N(C₁₋₈alkyl)₂, aminocarbonyl, hydroxyl, —NH-heteroaryl, —CF₃ or hydroxyl, optionally substituted with halogen, nitril, hydroxyl, C₁₋₈alkyl, SO₂, —OC₁₋₈alkyl, heterocyclyl, —CH₂F, C₃₋₁₀cycloalkyl, —NH₂, —NHC₁₋₈alkyl, —N(C₁₋₈alkyl)₂, C₂₋₁₀alkenyl or oxo (═O);
L₁ is —O—, —OC₁₋₈alkyl, heterocyclyl, or heteroaryl, optionally substituted with oxo (═O), —CF₃, —CH₂F, or C₃₋₁₀cycloalkyl;
R₁₁ is -3,8-diazabicyclo[3.2.1]octane,

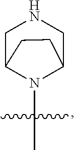 [SMILES: *N1C2CCC1CNC2]

and
R₁₂ is aryl, or heteroaryl, optionally substituted with hydroxyl, amino, halogen, C₁₋₈alkyl, CF₃, heteroaryl, C₃₋₁₀cycloalkyl, —NH₂, —NHC₁₋₈alkyl, —N(C₁₋₈alkyl)₂, or C₂₋₁₀alkenyl.

Aspect 25d: Provided herein are bridged compounds having the formula (VIII) and pharmaceutically acceptable salts, tautomers, isotopologues, stereoisomers and prodrugs thereof,
wherein R₄ is methyl, halogen;
R₅ is C₁₋₈alkyl, C₃₋₁₀cycloalkyl, C₂₋₁₀alkenyl, halogen, CF₃, or —S—CF₃, optionally substituted with one, two or three halogen;
R₇ is hydrogen;
R₈ is C₃₋₁₀cycloalkyl, heterocyclyl, heteroaryl, —NH₂, —NHC₁₋₈alkyl, —N(C₁₋₈alkyl)₂, aminocarbonyl, hydroxyl, —NH-heteroaryl, —CF₃ or hydroxyl, optionally substituted with halogen, nitril, hydroxyl, C₁₋₈alkyl, SO₂, —OC₁₋₈alkyl, heterocyclyl, —CH₂F, C₃₋₁₀cycloalkyl, —NH₂, —NHC₁₋₈alkyl, —N(C₁₋₈alkyl)₂, C₂₋₁₀alkenyl or oxo (═O);
L₁ is —O—, —OC₁₋₈alkyl, heterocyclyl, or heteroaryl, optionally substituted with oxo (═O), —CF₃, —CH₂F, or C₃₋₁₀cycloalkyl;
R₁₁ is -3,8-diazabicyclo[3.2.1]octane, or

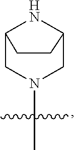 [SMILES: *N1CC2CCC(C1)N2]

and
R₁₂ is aryl, or heteroaryl, optionally substituted with hydroxyl, amino, halogen, C₁₋₈alkyl, CF₃, heteroaryl, C₃₋₁₀cycloalkyl, —NH₂, —NHC₁₋₈alkyl, —N(C₁₋₈alkyl)₂, or C₂₋₁₀alkenyl.

Aspect 26: Provided herein are bridged compounds having the formula (VIII) and pharmaceutically acceptable salts, tautomers, isotopologues, stereoisomers and prodrugs thereof,
wherein R₄ is chloro, fluoro;
R₅ is C₁₋₈alkyl, C₃₋₁₀cycloalkyl, C₂₋₁₀alkenyl, chloro, CF₃, or —S—CF₃, optionally substituted with one, two or three fluoro;
R₇ is hydrogen,
R₈ is C₃₋₁₀cycloalkyl, heterocyclyl, heteroaryl, —NH₂, —NHC₁₋₈alkyl, —N(C₁₋₈alkyl)₂, aminocarbonyl, hydroxyl, —NH-heteroaryl, —CF₃ or hydroxyl, optionally substituted with fluoro, nitril, hydroxyl, C₁₋₈alkyl, SO₂, —OC₁₋₈alkyl, heterocyclyl, —CH₂F, C₃₋₁₀cycloalkyl, —NH₂, —NHC₁₋₈alkyl, —N(C₁₋₈alkyl)₂, C₂₋₁₀alkenyl or oxo (═O);
L₁ is —O—, —OC₁₋₈alkyl, heterocyclyl, or heteroaryl, optionally substituted with oxo (═O), —CF₃, —CH₂F, or C₃₋₁₀cycloalkyl;
R₁₁ is 3-oxa-7,9-diazabicyclo[3.3.1]nonane,

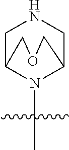 [SMILES: *N1C2CNCC1COC2]

3,6-diazabicyclo[3.1.1]heptane,

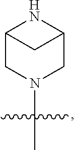 [SMILES: *N1CC2CC(C1)N2]

2,5-diazabicyclo[2.2.2]octane

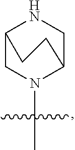 [SMILES: *N1CC2CCC1CN2]

2,5-diazabicyclo[2.2.1]heptane,

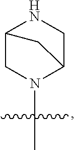 [SMILES: *N1CC2CC1CN2]

7-azabicyclo[2.2.1]heptan-7-yl,

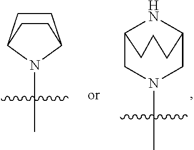 [SMILES: *N1C2CCC1CC2 ; *N1CC2CCCC(C1)N2]

optionally substituted with —NH₂, —NHR_{Y1b}, or —N(R_{Y1b})₂, where R_{Y1b} is C₁₋₈alkyl optionally substituted with one, two or three fluoro or R_{Y1b} is C₁₋₈alkyl optionally substituted with one —OCH₃; and
R₁₂ is aryl, or heteroaryl, optionally substituted with hydroxyl, amino, halogen, C₁₋₈alkyl, CF₃, heteroaryl, C₃₋₁₀cycloalkyl, —NH₂, —NHC₁₋₈alkyl, —N(C₁₋₈alkyl)₂, or C₂₋₁₀alkenyl.

Aspect 26b: Provided herein are bridged compounds having the formula (VIII) and pharmaceutically acceptable salts, tautomers, isotopologues, stereoisomers and prodrugs thereof,
wherein R₄ is chloro, fluoro;
R₅ is C₁₋₈alkyl, C₃₋₁₀cycloalkyl, C₂₋₁₀alkenyl, chloro, CF₃, or —S—CF₃, optionally substituted with one, two or three fluoro;
R₇ is hydrogen,
-L₁-R₈

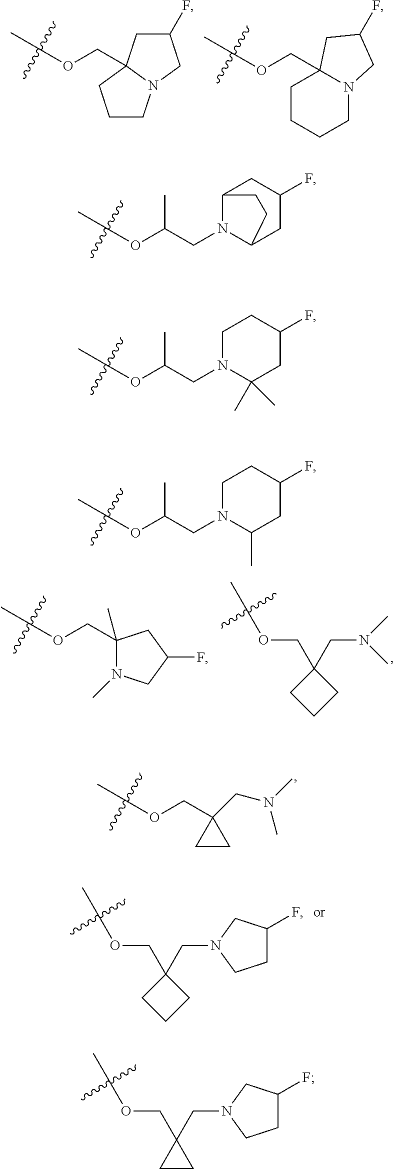

R₁₁ is 3-oxa-7,9-diazabicyclo[3.3.1]nonane,

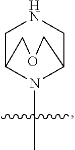 [SMILES: *N1C2CNCC1COC2]

3,6-diazabicyclo[3.1.1]heptane,

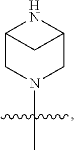 [SMILES: *N1CC2CC(C1)N2]

2,5-diazabicyclo[2.2.2]octane,

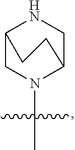 [SMILES: *N1CC2CCC1CN2]

2,5-diazabicyclo[2.2.1]heptane,

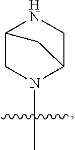 [SMILES: *N1CC2CC1CN2]

7-azabicyclo[2.2.1]heptan-7-yl,

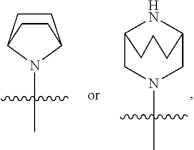 [SMILES: *N1C2CCC1CC2 ; *N1CC2CCCC(C1)N2]

optionally substituted with —NH₂, —NHR_{Y1b}, or —N(R_{Y1b})₂, where R_{Y1b} is C₁₋₈alkyl optionally substituted with one, two or three fluoro or R_{Y1b} is C₁₋₈alkyl optionally substituted with one —OCH₃; and
R₁₂ is aryl, or heteroaryl, optionally substituted with hydroxyl, amino, halogen, C₁₋₈alkyl, CF₃, heteroaryl, C₃₋₁₀cycloalkyl, —NH₂, —NHC₁₋₈alkyl, —N(C₁₋₈alkyl)₂, or C₂₋₁₀alkenyl.

Aspect 26c: Provided herein are bridged compounds having the formula (VIII) and pharmaceutically acceptable salts, tautomers, isotopologues, stereoisomers and prodrugs thereof,
wherein R₄ is chloro, fluoro;
R₅ is C₁₋₈alkyl, C₃₋₁₀cycloalkyl, C₂₋₁₀alkenyl, chloro, CF₃, or —S—CF₃, optionally substituted with one, two or three fluoro;
R₇ is hydrogen,
R₈ is C₃₋₁₀cycloalkyl, heterocyclyl, heteroaryl, —NH₂, —NHC₁₋₈alkyl, —N(C₁₋₈alkyl)₂, aminocarbonyl, hydroxyl, —NH-heteroaryl, —CF₃ or hydroxyl, optionally substituted with fluoro, nitril, hydroxyl, C₁₋₈alkyl, SO₂, —OC₁₋₈alkyl, heterocyclyl, —CH₂F, C₃₋₁₀cycloalkyl, —NH₂, —NHC₁₋₈alkyl, —N(C₁₋₈alkyl)₂, C₂₋₁₀alkenyl or oxo (═O);
L₁ is —O—, —OC₁₋₈alkyl, heterocyclyl, or heteroaryl, optionally substituted with oxo (═O), —CF₃, —CH₂F, or C₃₋₁₀cycloalkyl;
R₁₁ is 3-oxa-7,9-diazabicyclo[3.3.1]nonane,

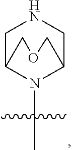 [SMILES: *N1C2CNCC1COC2]

3,6-diazabicyclo[3.1.1]heptane,

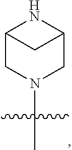 [SMILES: *N1CC2CC(C1)N2]

2,5-diazabicyclo[2.2.2]octane,

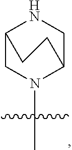 [SMILES: *N1CC2CCC1CN2]

2,5-diazabicyclo[2.2.1]heptane,

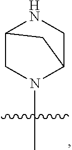 [SMILES: *N1CC2CC1CN2]

7-azabicyclo[2.2.1]heptan-7-yl,

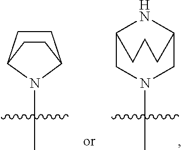 [SMILES: *N1C2CCC1CC2 ; *N1CC2CCCC(C1)N2]

optionally substituted with —NH₂, —NHR_{Y1b}, or —N(R_{Y1b})₂, where R_{Y1b} is C₁₋₈alkyl optionally substituted with one, two or three fluoro or R_{Y1b} is C₁₋₈alkyl optionally substituted with one —OCH₃; and
R₁₂ is

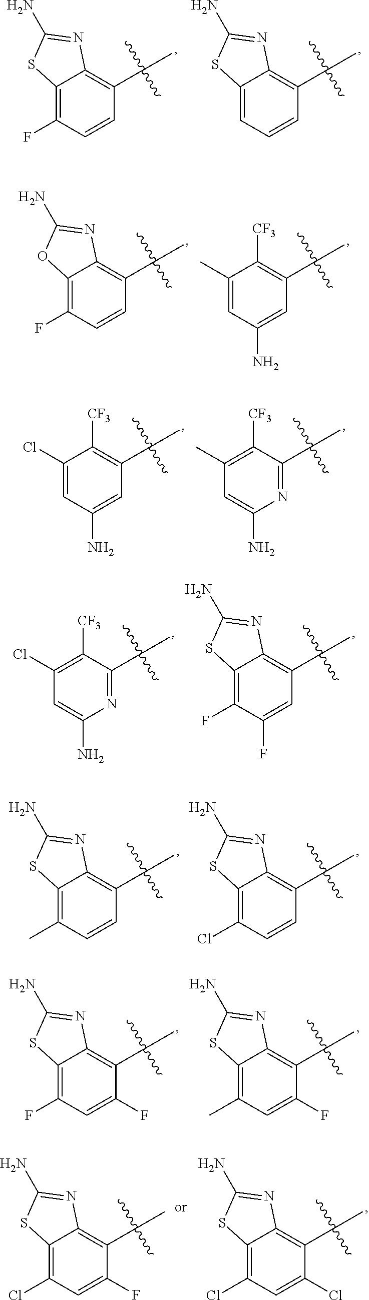

optionally substituted with hydroxyl, amino, halogen, C₁₋₈alkyl, CF₃, heteroaryl, C₃₋₁₀cycloalkyl, —NH₂, —NHC₁₋₈alkyl, —N(C₁₋₈alkyl)₂, or C₂₋₁₀alkenyl.

Aspect 26d: Provided herein are bridged compounds having the formula (VIII) and pharmaceutically acceptable salts, tautomers, isotopologues, stereoisomers and prodrugs thereof,
wherein R₄ is fluoro;
R₅ is C₁₋₈alkyl, C₃₋₁₀cycloalkyl, C₂₋₁₀alkenyl, chloro, CF₃, or —S—CF₃, optionally substituted with one, two or three fluoro;
R₇ is hydrogen;
-L₁-R₈ is

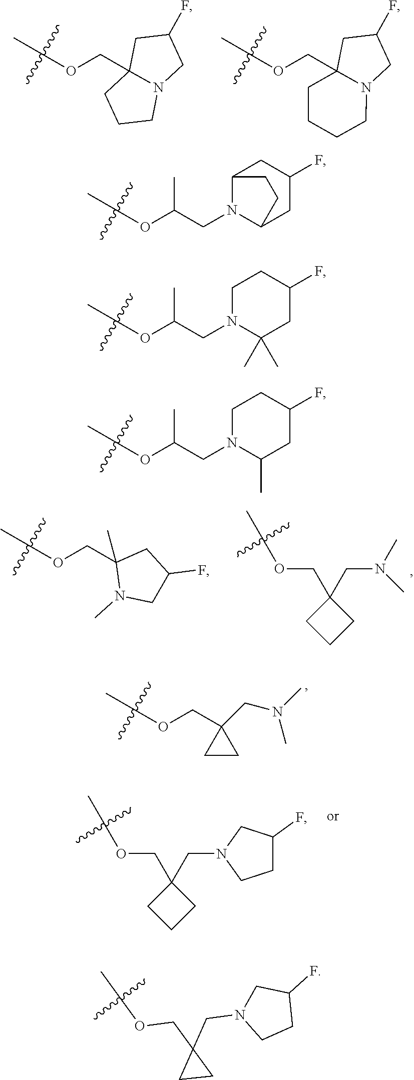

R₁₁ is 3-oxa-7,9-diazabicyclo[3.3.1]nonane,

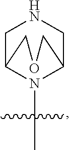

3,6-diazabicyclo[3.1.1]heptane,

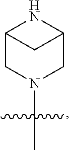

2,5-diazabicyclo[2.2.2]octane,

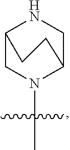

2,5-diazabicyclo[2.2.1]heptane,

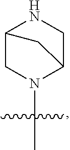

7-azabicyclo[2.2.1]heptan-7-yl,

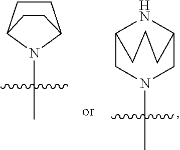

optionally substituted with —NH₂, —NHR_{Y1b}, or —N(R_{Y1b})₂, where R_{Y1b} is C₁₋₈alkyl optionally substituted with one, two or three fluoro or R_{Y1b} is C₁₋₈alkyl optionally substituted with one —OCH₃; and
R₁₂ is

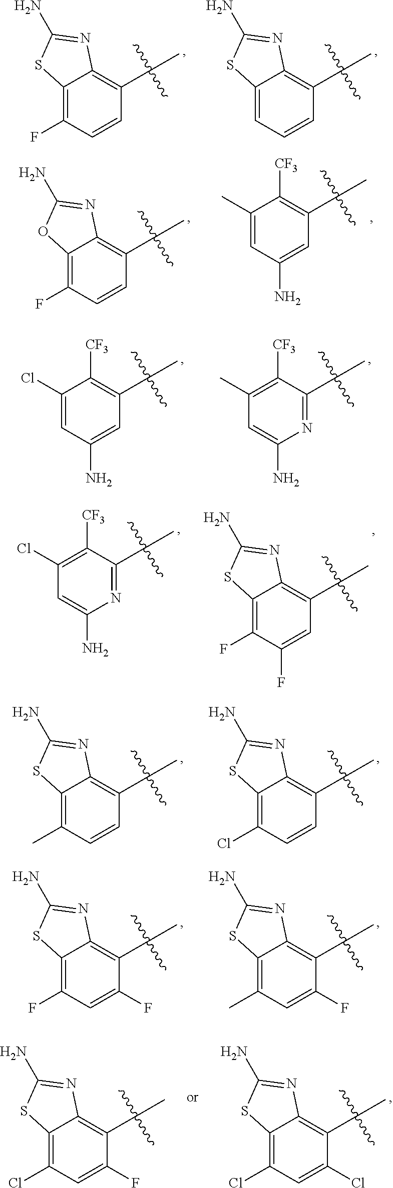

optionally substituted with hydroxyl, amino, halogen, C₁₋₈alkyl, CF₃, heteroaryl, C₃₋₁₀cycloalkyl, —NH₂, —NHC₁₋₈alkyl, —N(C₁₋₈alkyl)₂, or C₂₋₁₀alkenyl.

Aspect 27: Provided herein are bridged compounds having the formula (VIII) and pharmaceutically acceptable salts, tautomers, isotopologues, stereoisomers and prodrugs thereof,
wherein R₄ is halogen;
R₅ is C₁₋₈alkyl, C₃₋₁₀cycloalkyl, C₂₋₁₀alkenyl, halogen, CF₃, or —S—CF₃, optionally substituted with one, two or three halogen;
R₇ is hydrogen;
R₈ is C₃₋₁₀cycloalkyl, heterocyclyl, heteroaryl, —NH₂, —NHC₁₋₈alkyl, —N(C₁₋₈alkyl)₂, aminocarbonyl, hydroxyl, —NH-heteroaryl, —CF₃ or hydroxyl, optionally substituted with halogen, nitril, hydroxyl, C₁₋₈alkyl, SO₂, —OC₁₋₈alkyl, heterocyclyl, —CH₂F, C₃₋₁₀cycloalkyl, —NH₂, —NHC₁₋₈alkyl, —N(C₁₋₈alkyl)₂, C₂₋₁₀alkenyl or oxo (═O);
L₁ is —O—, —OC₁₋₈alkyl, heterocyclyl, or heteroaryl, optionally substituted with oxo (═O), —CF₃, —CH₂F, or C₃₋₁₀cycloalkyl;
R₁₁ is 3-oxa-7,9-diazabicyclo[3.3.1]nonane,

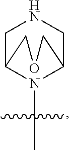

3,6-diazabicyclo[3.1.1]heptane,

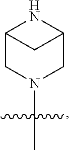

2,5-diazabicyclo[2.2.2]octane,

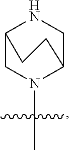

2,5-diazabicyclo[2.2.1]heptane,

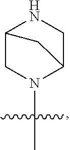

7-azabicyclo[2.2.1]heptan-7-yl,

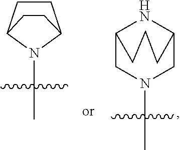

optionally substituted with —NH₂, —NHR_{Y1b}, or —N(R_{Y1b})₂, where R_{Y1b} is C₁₋₈alkyl optionally substituted with one, two or three halogen or R_{Y1b} is C₁₋₈alkyl optionally substituted with one —OCH₃; and
R₁₂ is 2-amino-7-fluorobenzo[d]thiazolyl or

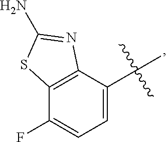

2-amino-5,7-fluorobenzo[d]thiazolyl or

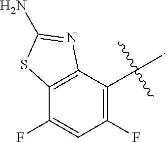

Aspect 28: Provided herein are bridged compounds having the formula (VIII) and pharmaceutically acceptable salts, tautomers, isotopologues, stereoisomers and prodrugs thereof,
wherein R₄ is chloro, fluoro;
R₅ is C₁₋₈alkyl, C₃₋₁₀cycloalkyl, C₂₋₁₀alkenyl, chloro, CF₃, or —S—CF₃, optionally substituted with one, two or three fluoro;
R₇ is hydrogen,
R₈ is C₃₋₁₀cycloalkyl, heterocyclyl, heteroaryl, —NH₂, —NHC₁₋₈alkyl, —N(C₁₋₈alkyl)₂, aminocarbonyl, hydroxyl, —NH-heteroaryl, —CF₃ or hydroxyl, optionally substituted with fluoro, nitril, hydroxyl, C₁₋₈alkyl, SO₂, —OC₁₋₈alkyl, heterocyclyl, —CH₂F, C₃₋₁₀cycloalkyl, —NH₂, —NHC₁₋₈alkyl, —N(C₁₋₈alkyl)₂, C₂₋₁₀alkenyl or oxo (═O);
L₁ is —O—, —OC₁₋₈alkyl, heterocyclyl, or heteroaryl, optionally substituted with oxo (═O), —CF₃, —CH₂F, or C₃₋₁₀cycloalkyl;
R₁₁ is 3-oxa-7,9-diazabicyclo[3.3.1]nonane,

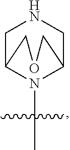

3,6-diazabicyclo[3.1.1]heptane,

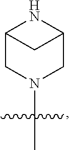

2,5-diazabicyclo[2.2.2]octane,

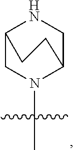 [SMILES: *N1CC2CCC1CN2]

2,5-diazabicyclo[2.2.1]heptane,

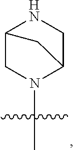 [SMILES: *N1CC2CC1CN2]

7-azabicyclo[2.2.1]heptan-7-yl,

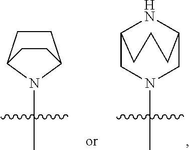 [SMILES: *N1C2CCC1CC2 ; *N1CC2CCCC(C1)N2]

optionally substituted with —NH₂, —NHR_{Y1b}, or —N(R_{Y1b})₂, where R_{Y1b} is C₁₋₈alkyl optionally substituted with one, two or three fluoro or R_{Y1b} is C₁₋₈alkyl optionally substituted with one —OCH₃; and
R₁₂ 2-amino-7-fluorobenzo[d]thiazol or

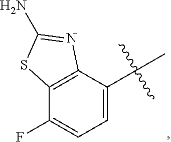 [SMILES: *c1ccc(F)c2sc(N)nc12]

2-amino-5,7-fluorobenzo[d]thiazol or

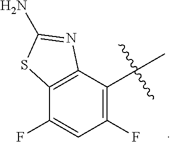 [SMILES: *c1c(F)cc(F)c2sc(N)nc12]

Aspect 29: Provided herein are bridged compounds having the formula (VIII) and pharmaceutically acceptable salts, tautomers, isotopologues, stereoisomers and prodrugs thereof,
wherein R₄ is halogen;
R₅ is C₁₋₈alkyl, C₃₋₁₀cycloalkyl, C₂₋₁₀alkenyl, halogen, CF₃, or —S—CF₃, optionally substituted with one, two or three halogen;
R₇ is hydrogen;
R₈ is (2R,7aS)-2-fluorotetrahydro-1H-pyrrolizin-7a(5H)-yl or

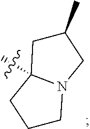

L₁ is —OCH₂—;
R₁₁ is 3-oxa-7,9-diazabicyclo[3.3.1]nonane,

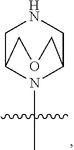 [SMILES: *N1C2CNCC1COC2]

3,6-diazabicyclp[3.1.1]heptane,

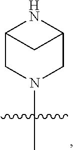 [SMILES: *N1CC2CC(C1)N2]

2,5-diazabicyclo[2.2.2]octane,

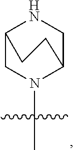 [SMILES: *N1CC2CCC1CN2]

2,5-diazabicyclo[2.2.1]heptane,

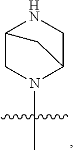 [SMILES: *N1CC2CC1CN2]

7-azabicyclo[2.2.1]heptan-7-yl,

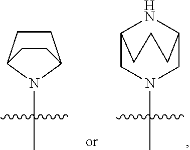 [SMILES: *N1C2CCC1CC2 ; *N1CC2CCCC(C1)N2]

optionally substituted with —NH₂, —NHR_{Y1b}, or —N(R_{Y1b})₂, where R_{Y1b} is C₁₋₈alkyl optionally substituted with one, two or three halogen or R_{Y1b} is C₁₋₈alkyl optionally substituted with one —OCH₃; and
R₁₂ is aryl, or heteroaryl, optionally substituted with hydroxyl, amino, halogen, C₁₋₈alkyl, CF₃, heteroaryl, C₃₋₁₀cycloalkyl, —NH₂, —NHC₁₋₈alkyl, —N(C₁₋₈alkyl)₂, or C₂₋₁₀alkenyl.

Aspect 30: Provided herein are bridged compounds having the formula (VIII) and pharmaceutically acceptable salts, tautomers, isotopologues, stereoisomers and prodrugs thereof,
wherein R₄ is chloro, fluoro;
R₅ is C₁₋₈alkyl, C₃₋₁₀cycloalkyl, C₂₋₁₀alkenyl, chloro, CF₃, or —S—CF₃, optionally substituted with one, two or three fluoro;
R₇ is hydrogen,
R₈ is (2R,7aS)-2-fluorotetrahydro-1H-pyrrolizin-7a(5H)-yl or

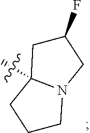

L₁ is —OCH₂—;
R₁₁ is 3-oxa-7,9-diazabicyclo[3.3.1]nonane,

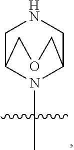 [SMILES: *N1C2CNCC1COC2]

3,6-diazabicyclo[3.1.1]heptane,

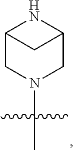 [SMILES: *N1CC2CC(C1)N2]

2,5-diazabicyclo[2.2.2]octane,

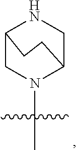 [SMILES: *N1CC2CCC1CN2]

2,5-diazabicyclo[2.2.1]heptane,

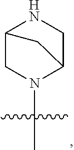 [SMILES: *N1CC2CC1CN2]

7-azabicyclo[2.2.1]heptan-7-yl,

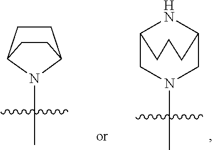 [SMILES: *N1C2CCC1CC2 ; *N1CC2CCCC(C1)N2]

optionally substituted with —NH₂, —NHR_{Y1b}, or —N(R_{Y1b})₂, where R_{Y1b} is C₁₋₈alkyl optionally substituted with one, two or three fluoro or R_{Y1b} is C₁₋₈alkyl optionally substituted with one —OCH₃; and
R₁₂ is aryl, or heteroaryl, optionally substituted with hydroxyl, amino, halogen, C₁₋₈alkyl, CF₃, heteroaryl, C₃₋₁₀cycloalkyl, —NH₂, —NHC₁₋₈alkyl, —N(C₁₋₈alkyl)₂, or C₂₋₁₀alkenyl.

Aspect 31: Provided herein are bridged compounds having the formula (VIII) and pharmaceutically acceptable salts, tautomers, isotopologues, stereoisomers and prodrugs thereof,
wherein R₄ is halogen;
R₅ is C₁₋₈alkyl, C₃₋₁₀cycloalkyl, C₂₋₁₀alkenyl, halogen, CF₃, or —S—CF₃, optionally substituted with one, two or three halogen;
R₇ is hydrogen;
R₈ is (2R,7aS)-2-fluorotetrahydro-1H-pyrrolizin-7a(5H)-yl or

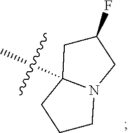

L₁ is —OCH₂—;
R₁₁ is 3-oxa-7,9-diazabicyclo[3.3.1]nonane,

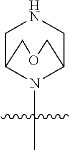 [SMILES: *N1C2CNCC1COC2]

3,6-diazabicyclo[3.1.1]heptane,

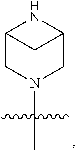 [SMILES: *N1CC2CC(C1)N2]

2,5-diazabicyclo[2.2.2]octane,

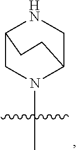 [SMILES: *N1CC2CCC1CN2]

2,5-diazabicyclo[2.2.1]heptane,

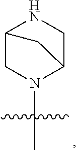 [SMILES: *N1CC2CC1CN2]

7-azabicyclo[2.2.1]heptan-7-yl,

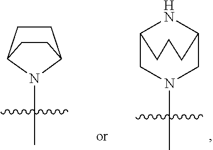 [SMILES: *N1C2CCC1CC2 ; *N1CC2CCCC(C1)N2]

optionally substituted with —NH₂, —NHR_{Y1b}, or —N(R_{Y1b})₂, where R_{Y1b} is C₁₋₈alkyl optionally substituted with one, two or three halogen or R_{Y1b} is C₁₋₈alkyl optionally substituted with one —OCH₃; and
R₁₂ is 2-amino-7-fluorobenzo[d]thiazol or

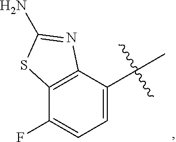 [SMILES: *c1ccc(F)c2sc(N)nc12]

2-amino-5,7-fluorobenzo[d]thiazol or

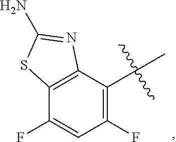 [SMILES: *c1c(F)cc(F)c2sc(N)nc12]

Aspect 32: Provided herein are bridged compounds having the formula (VIII) and pharmaceutically acceptable salts, tautomers, isotopologues, stereoisomers and prodrugs thereof,
wherein R₄ is fluoro;
R₅ is C₁₋₈alkyl, C₃₋₁₀cycloalkyl, C₂₋₁₀alkenyl, chloro, CF₃, or —S—CF₃, optionally substituted with one, two or three fluoro;
R₇ is hydrogen,
R₈ is (2R,7aS)-2-fluorotetrahydro-1H-pyrrolizin-7a(5H)-yl or

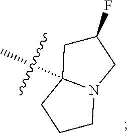

L₁ is —OCH₂—;
R₁₁ is 3-oxa-7,9-diazabicyclo[3.3.1]nonane,

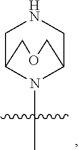 [SMILES: *N1C2CNCC1COC2]

3,6-diazabicyclo[3.1.1]heptane,

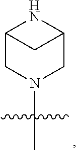 [SMILES: *N1CC2CC(C1)N2]

2,5-diazabicyclo[2.2.2]octane,

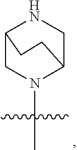 [SMILES: *N1CC2CCC1CN2]

2,5-diazabicyclo[2.2.1]heptane,

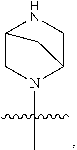 [SMILES: *N1CC2CC1CN2]

7-azabicyclo[2.2.1]heptan-7-yl,

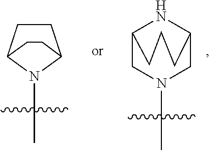 [SMILES: *N1C2CCC1CC2 ; *N1CC2CCCC(C1)N2]

optionally substituted with —NH₂, —NHR_{Y1b}, or —N(R_{Y1b})₂, where R_{Y1b} is C₁₋₈alkyl optionally substituted with one, two or three fluoro or R_{Y1b} is C₁₋₈alkyl optionally substituted with one —OCH₃; and
R₁₂ is 2-amino-7-fluorobenzo[d]thiazol or

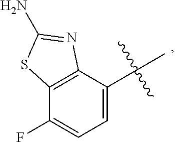 [SMILES: *c1ccc(F)c2sc(N)nc12]

2-amino-5,7-fluorobenzo[d]thiazol or

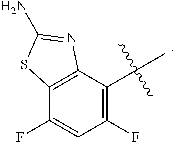 [SMILES: *c1c(F)cc(F)c2sc(N)nc12]

Aspect 33: Provided herein are bridged compounds having the formula (VIII) and pharmaceutically acceptable salts, tautomers, isotopologues, stereoisomers and prodrugs thereof,
wherein R₄ is halogen;
R₅ is —CF₃;
R₇ is hydrogen;
R₈ is (2R,7aS)-2-fluorotetrahydro-1H-pyrrolizin-7a(5H)-yl or

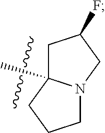

L₁ is —OCH₂—;
R₁₁ is 3-oxa-7,9-diazabicyclo[3.3.1]nonane or

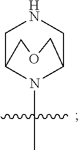 [SMILES: *N1C2CNCC1COC2]

and
R₁₂ is 2-amino-7-fluorobenzo[d]thiazol or

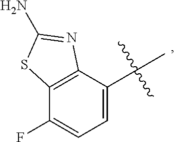 [SMILES: *c1ccc(F)c2sc(N)nc12]

2-amino-5,7-fluorobenzo[d]thiazol or

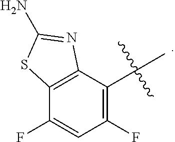 [SMILES: *c1c(F)cc(F)c2sc(N)nc12]

Aspect 34: Provided herein are bridged compounds having the formula (VIII) and pharmaceutically acceptable salts, tautomers, isotopologues, stereoisomers and prodrugs thereof,
wherein R₄ is chloro, fluoro;
R₅ is —CF₃;
R₇ is hydrogen,
R₈ is (2R,7aS)-2-fluorotetrahydro-1H-pyrrolizin-7a(5H)-yl or

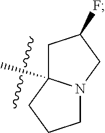

L₁ is —OCH₂—;
R₁₁ is 7-azabicyclo[2.2.1]heptan-7-yl or

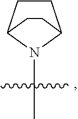 [SMILES: *N1C2CCC1CC2]

optionally substituted with —NH₂, —NHR_{Y1b}, or —N(R_{Y1b})₂, where R_{Y1b} is C₁₋₈alkyl optionally substituted with one, two or three fluoro or R_{Y1b} is C₁₋₈alkyl optionally substituted with one —OCH₃; and
R₂ is 2-amino-7-fluorobenzo[d]thiazol or

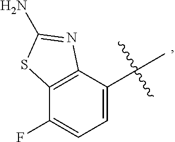 [SMILES: *c1ccc(F)c2sc(N)nc12]

2-amino-5,7-fluorobenzo[d]thiazol or

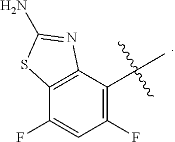 [SMILES: *c1c(F)cc(F)c2sc(N)nc12]

Aspect 34b: Provided herein are bridged compounds having the formula (VIII) and pharmaceutically acceptable salts, tautomers, isotopologues, stereoisomers and prodrugs thereof,
wherein R₄ is chloro, fluoro;
R₅ is —CF₃;
R₇ is hydrogen,
R₈ is (2R,7aS)-2-fluorotetrahydro-1H-pyrrolizin-7a(5H)-yl or

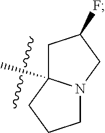

L₁ is —OCH₂—;
R₁₁ is

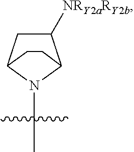

wherein R_{Y2a} and R_{Y2b} are independently hydrogen or C₁₋₈alkyl, wherein the C₁₋₈alkyl is optionally substituted with one, two or three fluoro or one —OCH₃; and
R₁₂ is 2-amino-7-fluorobenzo[d]thiazol,

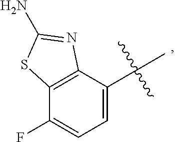 [SMILES: *c1ccc(F)c2sc(N)nc12]

2-amino-5,7-fluorobenzo[d]thiazol or

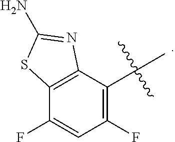 [SMILES: *c1c(F)cc(F)c2sc(N)nc12]

Aspect 35: Provided herein are bridged compounds having the formula (VIII) and pharmaceutically acceptable salts, tautomers, isotopologues, stereoisomers and prodrugs thereof,
wherein R₄ is chloro, fluoro;
R₅ is —CF₃;
R₇ is hydrogen,
R₈ is (2R,7aS)-2-fluorotetrahydro-1H-pyrrolizin-7a(5H)-yl or

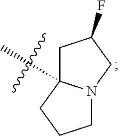

L₁ is —OCH₂—;
R₁₁ is 7-azabicyclo[2.2.1]heptan-7-yl or

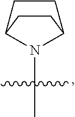 [SMILES: *N1C2CCC1CC2]

optionally substituted with —NH₂, —NHCH₃, —NHCH₂CH₂OCH₃, —NHCH₂CH₂CF₃, —NHCH₂CH₂CHF₂, —NHCH₂CHFCH₃, —NHCH₂CHF₂, —NHCH₂CH₂F; and
R₁₂ is 2-amino-7-fluorobenzo[d]thiazol or

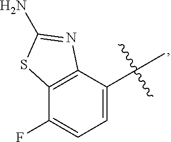 [SMILES: *c1ccc(F)c2sc(N)nc12]

2-amino-5,7-fluorobenzo[d]thiazol or

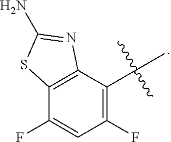 [SMILES: *c1c(F)cc(F)c2sc(N)nc12]

Aspect 36: Provided herein are bridged compounds having the formula (VIII) and pharmaceutically acceptable salts, tautomers, isotopologues, stereoisomers and prodrugs thereof,
wherein R₄ is chloro, fluoro;
R₅ is —CF₃;
R₇ is hydrogen,
R₈ is (2R,7aS)-2-fluorotetrahydro-1H-pyrrolizin-7a(5H)-yl or

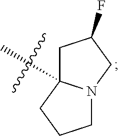

L₁ is —OCH₂—;
R₁₁ is 7-azabicyclo[2.2.1]heptan-7-yl or

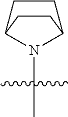 [SMILES: *N1C2CCC1CC2]

substituted with —NHCH₂CH₂OCH₃; and
R₁₂ is 2-amino-7-fluorobenzo[d]thiazol or

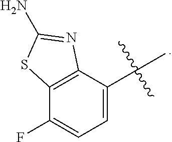 [SMILES: *c1ccc(F)c2sc(N)nc12]

Aspect 38 The compound of any one of Aspects 1-19, wherein the compound is selected from the following table:

TABLE 1

| | |
|---|---|
| 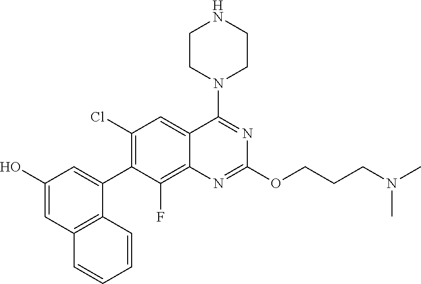 [SMILES: CN(C)CCCOc1nc(N2CCNCC2)c2cc(Cl)c(-c3cc(O)cc4ccccc34)c(F)c2n1] | E1 |
| 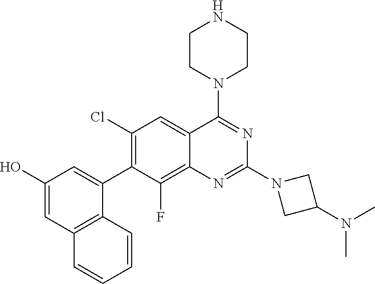 [SMILES: CN(C)C1CN(c2nc(N3CCNCC3)c3cc(Cl)c(-c4cc(O)cc5ccccc45)c(F)c3n2)C1] | E2 |
| 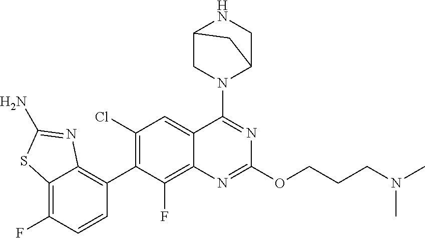 [SMILES: CN(C)CCCOc1nc(N2CC3CC2CN3)c2cc(Cl)c(-c3ccc(F)c4sc(N)nc34)c(F)c2n1] | E3 |
| 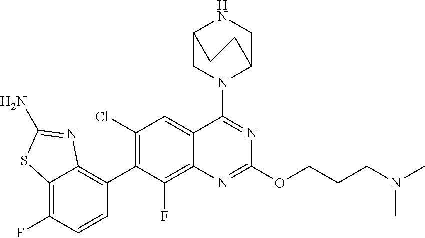 [SMILES: CN(C)CCCOc1nc(N2CC3CCC2CN3)c2cc(Cl)c(-c3ccc(F)c4sc(N)nc34)c(F)c2n1] | E4 |
| 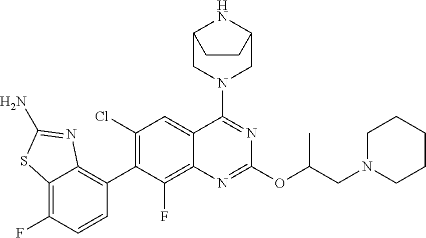 [SMILES: CC(CN1CCCCC1)Oc1nc(N2CC3CCC(C2)N3)c2cc(Cl)c(-c3ccc(F)c4sc(N)nc34)c(F)c2n1] | E5 |
| 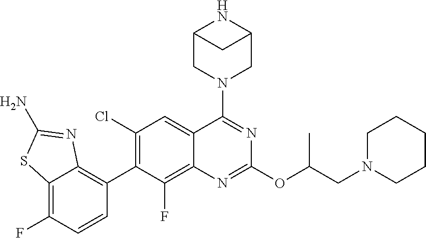 [SMILES: CC(CN1CCCCC1)Oc1nc(N2CC3CC(C2)N3)c2cc(Cl)c(-c3ccc(F)c4sc(N)nc34)c(F)c2n1] | E6 |
| 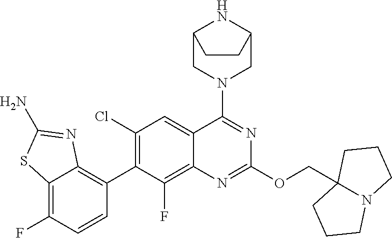 [SMILES: Nc1nc2c(-c3c(Cl)cc4c(N5CC6CCC(C5)N6)nc(OCC56CCCN5CCC6)nc4c3F)ccc(F)c2s1] | E7 |
| 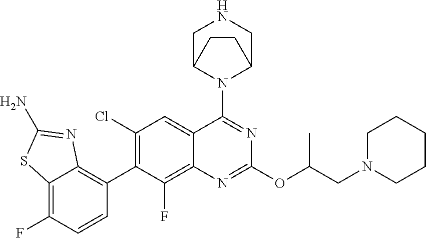 [SMILES: CC(CN1CCCCC1)Oc1nc(N2C3CCC2CNC3)c2cc(Cl)c(-c3ccc(F)c4sc(N)nc34)c(F)c2n1] | E8 |
| 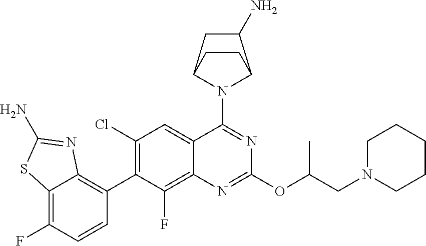 [SMILES: CC(CN1CCCCC1)Oc1nc(N2C3CCC2C(N)C3)c2cc(Cl)c(-c3ccc(F)c4sc(N)nc34)c(F)c2n1] | E9 |
| 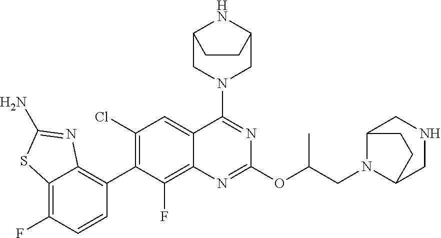 [SMILES: CC(CN1C2CCC1CNC2)Oc1nc(N2CC3CCC(C2)N3)c2cc(Cl)c(-c3ccc(F)c4sc(N)nc34)c(F)c2n1] | E10 |
| 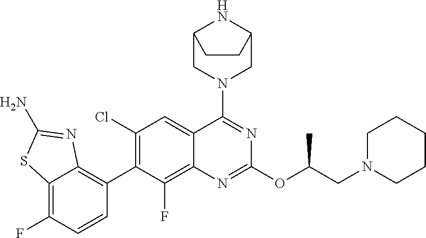 [SMILES: C[C@@H](CN1CCCCC1)Oc1nc(N2CC3CCC(C2)N3)c2cc(Cl)c(-c3ccc(F)c4sc(N)nc34)c(F)c2n1] | E11 |
| 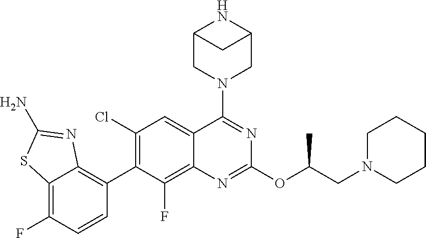 [SMILES: C[C@@H](CN1CCCCC1)Oc1nc(N2CC3CC(C2)N3)c2cc(Cl)c(-c3ccc(F)c4sc(N)nc34)c(F)c2n1] | E12 |
| 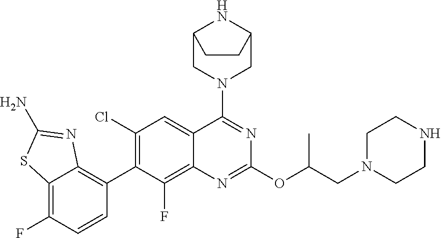 [SMILES: CC(CN1CCNCC1)Oc1nc(N2CC3CCC(C2)N3)c2cc(Cl)c(-c3ccc(F)c4sc(N)nc34)c(F)c2n1] | E13 |
| 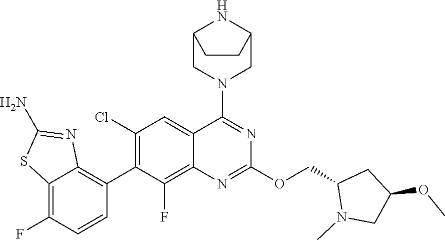 [SMILES: CO[C@@H]1C[C@@H](COc2nc(N3CC4CCC(C3)N4)c3cc(Cl)c(-c4ccc(F)c5sc(N)nc45)c(F)c3n2)N(C)C1] | E14 |
| 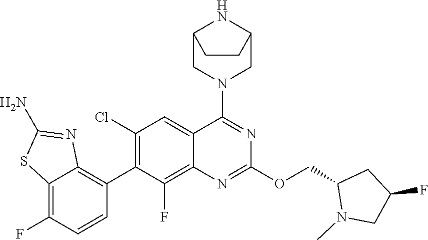 [SMILES: CN1C[C@H](F)C[C@H]1COc1nc(N2CC3CCC(C2)N3)c2cc(Cl)c(-c3ccc(F)c4sc(N)nc34)c(F)c2n1] | E15 |
| 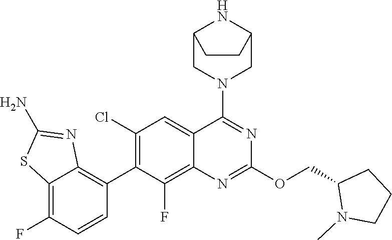 [SMILES: CN1CCC[C@H]1COc1nc(N2CC3CCC(C2)N3)c2cc(Cl)c(-c3ccc(F)c4sc(N)nc34)c(F)c2n1] | E16 |
| 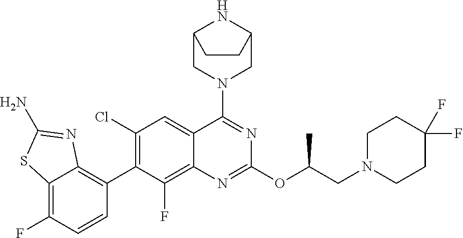 [SMILES: C[C@@H](CN1CCC(F)(F)CC1)Oc1nc(N2CC3CCC(C2)N3)c2cc(Cl)c(-c3ccc(F)c4sc(N)nc34)c(F)c2n1] | E17 |
| 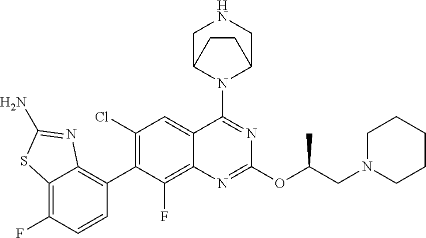 [SMILES: C[C@@H](CN1CCCCC1)Oc1nc(N2C3CCC2CNC3)c2cc(Cl)c(-c3ccc(F)c4sc(N)nc34)c(F)c2n1] | E18 |
| 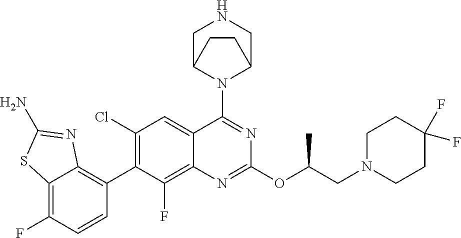 [SMILES: C[C@@H](CN1CCC(F)(F)CC1)Oc1nc(N2C3CCC2CNC3)c2cc(Cl)c(-c3ccc(F)c4sc(N)nc34)c(F)c2n1] | E19 |
| 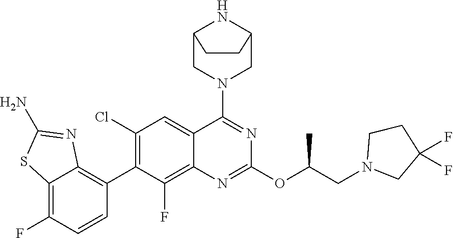 [SMILES: C[C@@H](CN1CCC(F)(F)C1)Oc1nc(N2CC3CCC(C2)N3)c2cc(Cl)c(-c3ccc(F)c4sc(N)nc34)c(F)c2n1] | E20 |
| 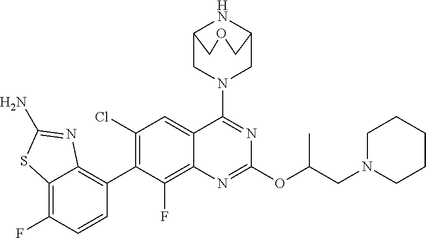 [SMILES: CC(CN1CCCCC1)Oc1nc(N2CC3COCC(C2)N3)c2cc(Cl)c(-c3ccc(F)c4sc(N)nc34)c(F)c2n1] | E21 |
| 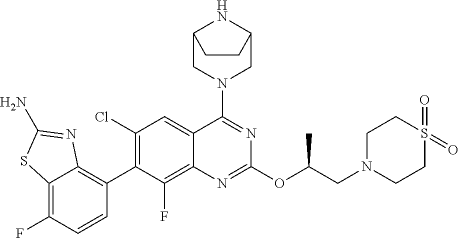 [SMILES: C[C@@H](CN1CCS(=O)(=O)CC1)Oc1nc(N2CC3CCC(C2)N3)c2cc(Cl)c(-c3ccc(F)c4sc(N)nc34)c(F)c2n1] | E22 |
| 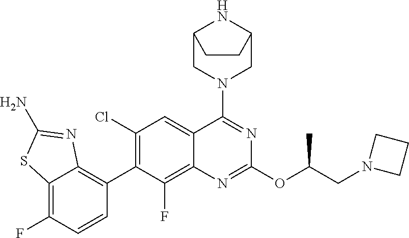 [SMILES: C[C@@H](CN1CCC1)Oc1nc(N2CC3CCC(C2)N3)c2cc(Cl)c(-c3ccc(F)c4sc(N)nc34)c(F)c2n1] | E23 |
| 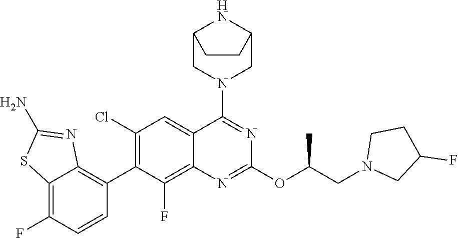 [SMILES: C[C@@H](CN1CCC(F)C1)Oc1nc(N2CC3CCC(C2)N3)c2cc(Cl)c(-c3ccc(F)c4sc(N)nc34)c(F)c2n1] | E24 |
| 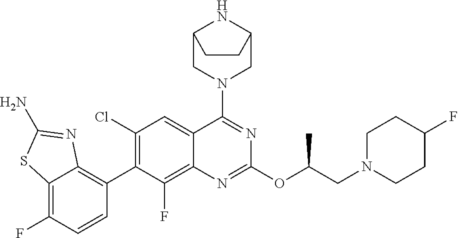 [SMILES: C[C@@H](CN1CCC(F)CC1)Oc1nc(N2CC3CCC(C2)N3)c2cc(Cl)c(-c3ccc(F)c4sc(N)nc34)c(F)c2n1] | E25 |
| 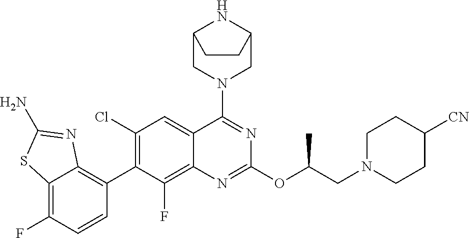 [SMILES: C[C@@H](CN1CCC(C#N)CC1)Oc1nc(N2CC3CCC(C2)N3)c2cc(Cl)c(-c3ccc(F)c4sc(N)nc34)c(F)c2n1] | E26 |
| 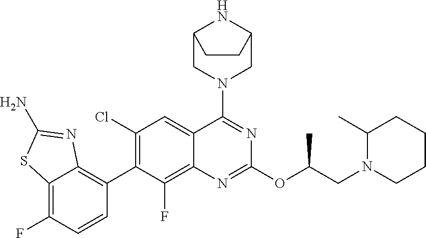 [SMILES: CC1CCCCN1C[C@H](C)Oc1nc(N2CC3CCC(C2)N3)c2cc(Cl)c(-c3ccc(F)c4sc(N)nc34)c(F)c2n1] | E27 |
| 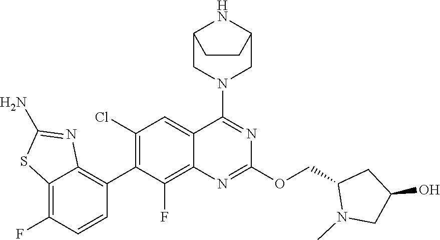 [SMILES: CN1C[C@H](O)C[C@H]1COc1nc(N2CC3CCC(C2)N3)c2cc(Cl)c(-c3ccc(F)c4sc(N)nc34)c(F)c2n1] | E28 |
| 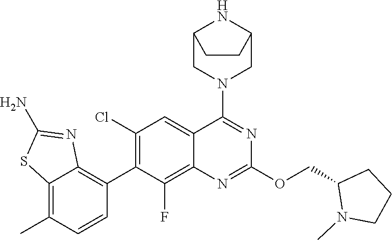 [SMILES: Cc1ccc(-c2c(Cl)cc3c(N4CC5CCC(C4)N5)nc(OC[C@@H]4CCCN4C)nc3c2F)c2nc(N)sc12] | |
| 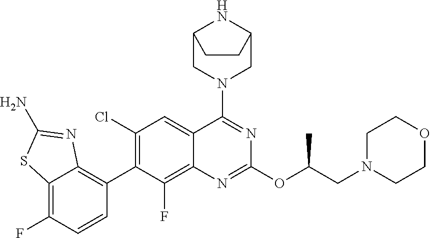 [SMILES: C[C@@H](CN1CCOCC1)Oc1nc(N2CC3CCC(C2)N3)c2cc(Cl)c(-c3ccc(F)c4sc(N)nc34)c(F)c2n1] | E29 |
| 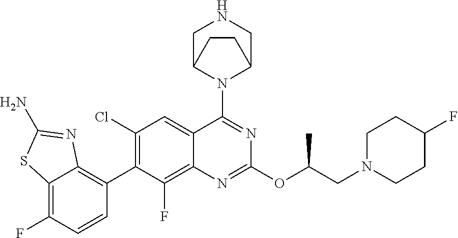 [SMILES: C[C@@H](CN1CCC(F)CC1)Oc1nc(N2C3CCC2CNC3)c2cc(Cl)c(-c3ccc(F)c4sc(N)nc34)c(F)c2n1] | E30 |
| 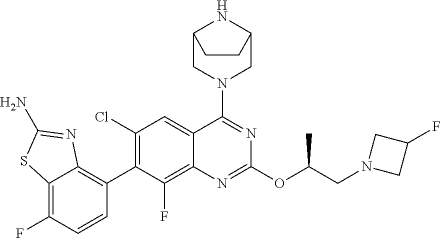 [SMILES: C[C@@H](CN1CC(F)C1)Oc1nc(N2CC3CCC(C2)N3)c2cc(Cl)c(-c3ccc(F)c4sc(N)nc34)c(F)c2n1] | E31 |
| 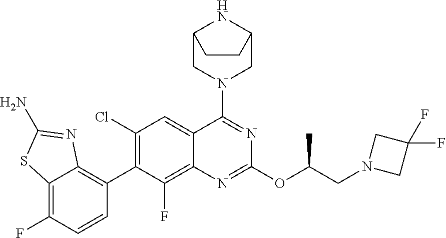 [SMILES: C[C@@H](CN1CC(F)(F)C1)Oc1nc(N2CC3CCC(C2)N3)c2cc(Cl)c(-c3ccc(F)c4sc(N)nc34)c(F)c2n1] | E32 |
| 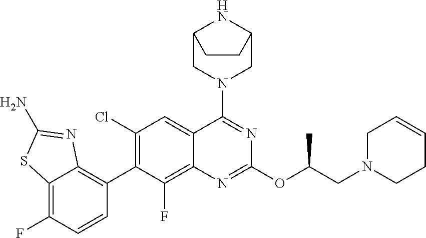 [SMILES: C[C@@H](CN1CC=CCC1)Oc1nc(N2CC3CCC(C2)N3)c2cc(Cl)c(-c3ccc(F)c4sc(N)nc34)c(F)c2n1] | E33 |
| 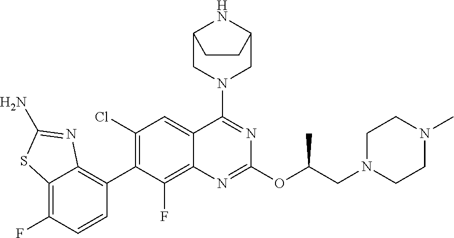 [SMILES: C[C@@H](CN1CCN(C)CC1)Oc1nc(N2CC3CCC(C2)N3)c2cc(Cl)c(-c3ccc(F)c4sc(N)nc34)c(F)c2n1] | E34 |
| 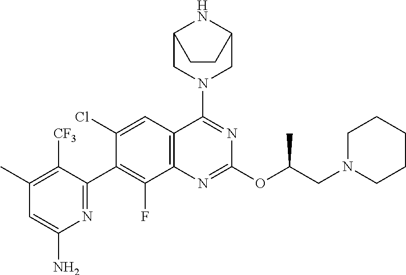 [SMILES: Cc1cc(N)nc(-c2c(Cl)cc3c(N4CC5CCC(C4)N5)nc(O[C@@H](C)CN4CCCCC4)nc3c2F)c1C(F)(F)F] | E35 |
| 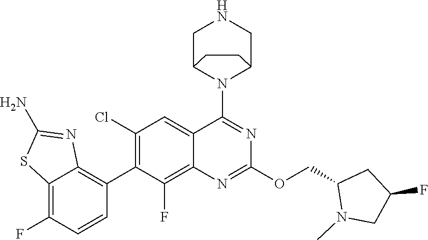 [SMILES: CN1C[C@H](F)C[C@H]1COc1nc(N2C3CCC2CNC3)c2cc(Cl)c(-c3ccc(F)c4sc(N)nc34)c(F)c2n1] | E36 |
| 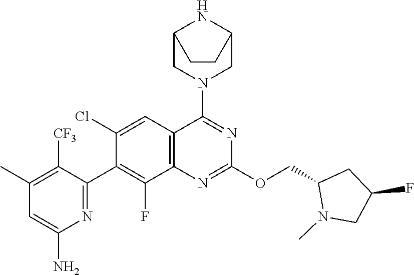 | E37 |
| 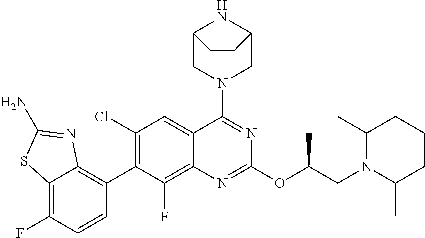 [SMILES: CC1CCCC(C)N1C[C@H](C)Oc1nc(N2CC3CCC(C2)N3)c2cc(Cl)c(-c3ccc(F)c4sc(N)nc34)c(F)c2n1] | E38 |
| 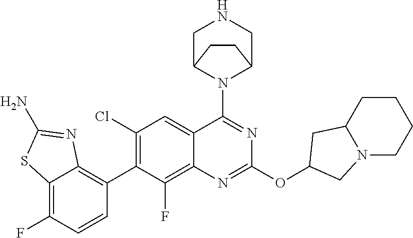 [SMILES: Nc1nc2c(-c3c(Cl)cc4c(N5C6CCC5CNC6)nc(OC5CC6CCCCN6C5)nc4c3F)ccc(F)c2s1] | E39 |
| 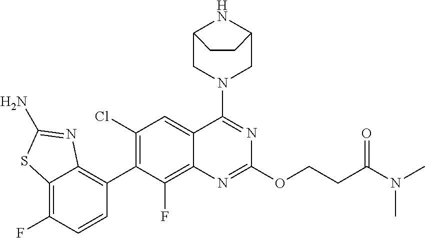 [SMILES: CN(C)C(=O)CCOc1nc(N2CC3CCC(C2)N3)c2cc(Cl)c(-c3ccc(F)c4sc(N)nc34)c(F)c2n1] | E40 |
| 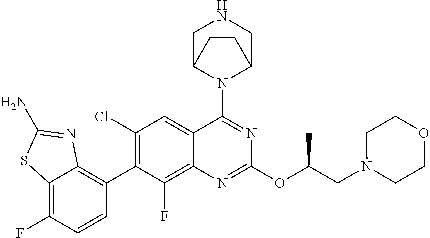 [SMILES: C[C@@H](CN1CCOCC1)Oc1nc(N2C3CCC2CNC3)c2cc(Cl)c(-c3ccc(F)c4sc(N)nc34)c(F)c2n1] | E41 |
| 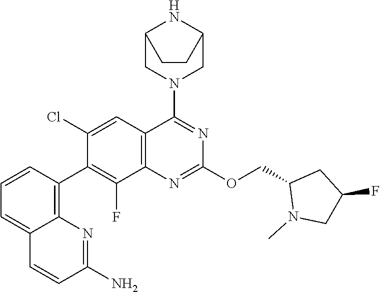 [SMILES: CN1C[C@H](F)C[C@H]1COc1nc(N2CC3CCC(C2)N3)c2cc(Cl)c(-c3cccc4ccc(N)nc34)c(F)c2n1] | E42 |
| 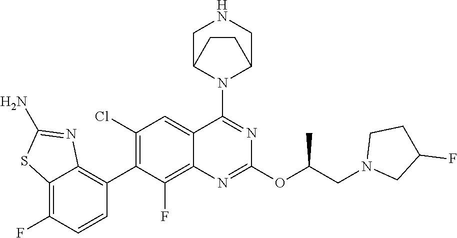 [SMILES: C[C@@H](CN1CCC(F)C1)Oc1nc(N2C3CCC2CNC3)c2cc(Cl)c(-c3ccc(F)c4sc(N)nc34)c(F)c2n1] | E43 |
| 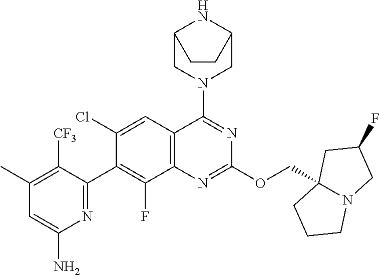 [SMILES: Cc1cc(N)nc(-c2c(Cl)cc3c(N4CC5CCC(C4)N5)nc(OC[C@@]45CCCN4C[C@H](F)C5)nc3c2F)c1C(F)(F)F] | E49 |
| 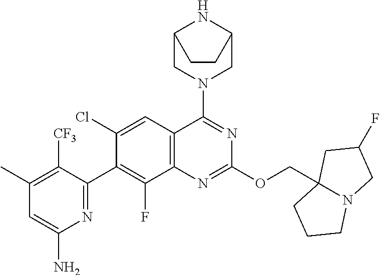 [SMILES: Cc1cc(N)nc(-c2c(Cl)cc3c(N4CC5CCC(C4)N5)nc(OCC45CCCN4CC(F)C5)nc3c2F)c1C(F)(F)F] | E50 |
| 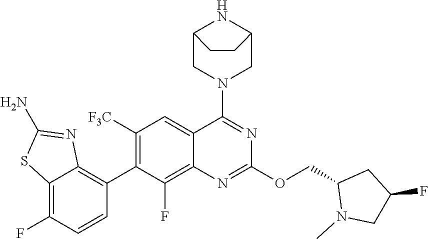 [SMILES: CN1C[C@H](F)C[C@H]1COc1nc(N2CC3CCC(C2)N3)c2cc(C(F)(F)F)c(-c3ccc(F)c4sc(N)nc34)c(F)c2n1] | E51 |
| 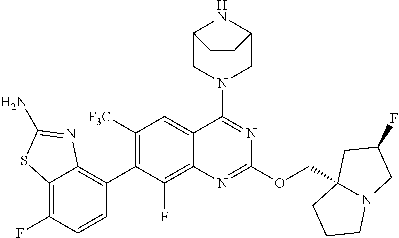 [SMILES: Nc1nc2c(-c3c(C(F)(F)F)cc4c(N5CC6CCC(C5)N6)nc(OC[C@@]56CCCN5C[C@H](F)C6)nc4c3F)ccc(F)c2s1] | E52 |
| 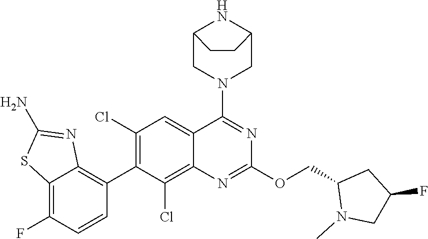 [SMILES: CN1C[C@H](F)C[C@H]1COc1nc(N2CC3CCC(C2)N3)c2cc(Cl)c(-c3ccc(F)c4sc(N)nc34)c(Cl)c2n1] | E53 |
| 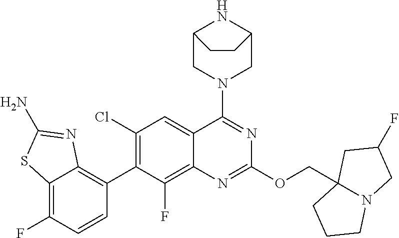 [SMILES: Nc1nc2c(-c3c(Cl)cc4c(N5CC6CCC(C5)N6)nc(OCC56CCCN5CC(F)C6)nc4c3F)ccc(F)c2s1] | E54 |
| 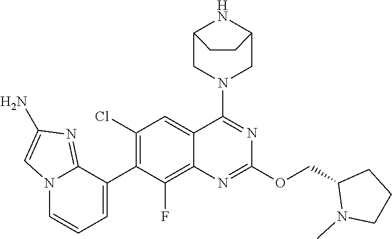 [SMILES: CN1CCC[C@H]1COc1nc(N2CC3CCC(C2)N3)c2cc(Cl)c(-c3cccn4cc(N)nc34)c(F)c2n1] | |
| 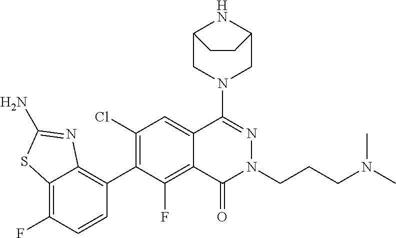 [SMILES: CN(C)CCCn1nc(N2CC3CCC(C2)N3)c2cc(Cl)c(-c3ccc(F)c4sc(N)nc34)c(F)c2c1=O] | |
| 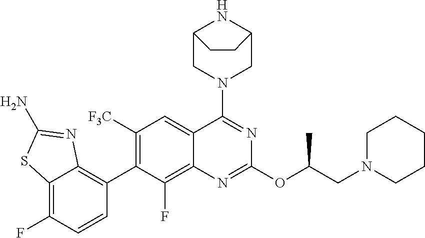 [SMILES: C[C@@H](CN1CCCCC1)Oc1nc(N2CC3CCC(C2)N3)c2cc(C(F)(F)F)c(-c3ccc(F)c4sc(N)nc34)c(F)c2n1] | |
| 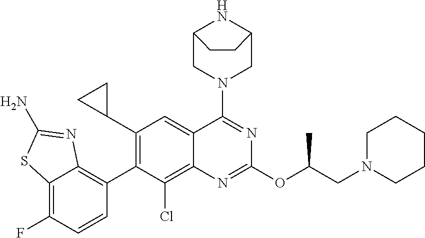 [SMILES: C[C@@H](CN1CCCCC1)Oc1nc(N2CC3CCC(C2)N3)c2cc(C3CC3)c(-c3ccc(F)c4sc(N)nc34)c(Cl)c2n1] | |
| 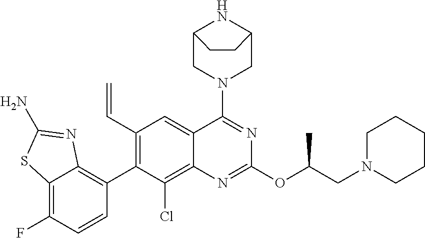 [SMILES: C=Cc1cc2c(N3CC4CCC(C3)N4)nc(O[C@@H](C)CN3CCCCC3)nc2c(Cl)c1-c1ccc(F)c2sc(N)nc12] | |
| 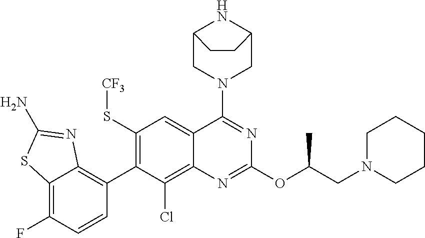 [SMILES: C[C@@H](CN1CCCCC1)Oc1nc(N2CC3CCC(C2)N3)c2cc(SC(F)(F)F)c(-c3ccc(F)c4sc(N)nc34)c(Cl)c2n1] | |
| 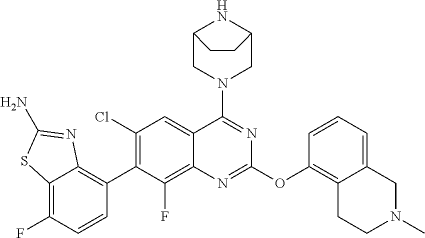 [SMILES: CN1CCc2c(cccc2Oc2nc(N3CC4CCC(C3)N4)c3cc(Cl)c(-c4ccc(F)c5sc(N)nc45)c(F)c3n2)C1] | |
| 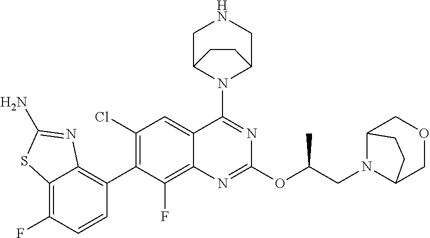 [SMILES: C[C@@H](CN1C2CCC1COC2)Oc1nc(N2C3CCC2CNC3)c2cc(Cl)c(-c3ccc(F)c4sc(N)nc34)c(F)c2n1] | |
| 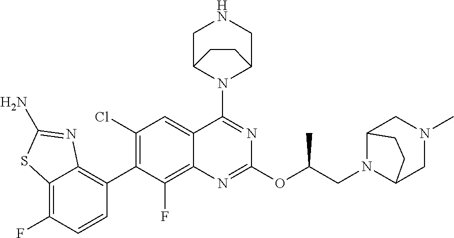 [SMILES: C[C@@H](CN1C2CCC1CN(C)C2)Oc1nc(N2C3CCC2CNC3)c2cc(Cl)c(-c3ccc(F)c4sc(N)nc34)c(F)c2n1] | |
| 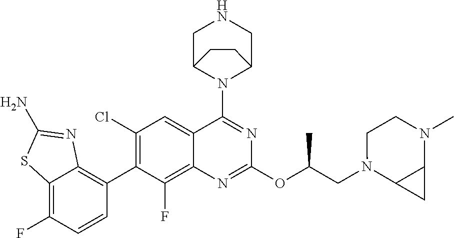 [SMILES: C[C@@H](CN1CCN(C)C2CC21)Oc1nc(N2C3CCC2CNC3)c2cc(Cl)c(-c3ccc(F)c4sc(N)nc34)c(F)c2n1] | |
| 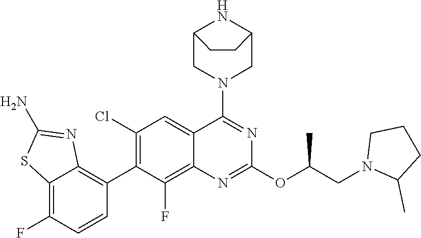 [SMILES: CC1CCCN1C[C@H](C)Oc1nc(N2CC3CCC(C2)N3)c2cc(Cl)c(-c3ccc(F)c4sc(N)nc34)c(F)c2n1] | |
| 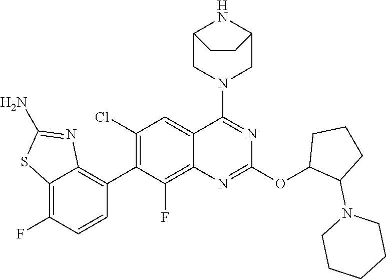 [SMILES: Nc1nc2c(-c3c(Cl)cc4c(N5CC6CCC(C5)N6)nc(OC5CCCC5N5CCCCC5)nc4c3F)ccc(F)c2s1] | |
| 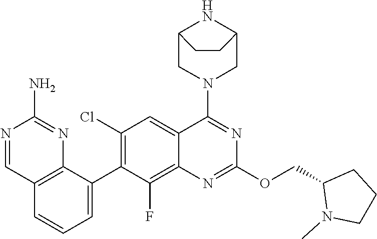 [SMILES: CN1CCC[C@H]1COc1nc(N2CC3CCC(C2)N3)c2cc(Cl)c(-c3cccc4cnc(N)nc34)c(F)c2n1] | |
| 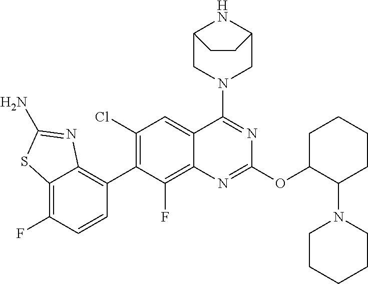 [SMILES: Nc1nc2c(-c3c(Cl)cc4c(N5CC6CCC(C5)N6)nc(OC5CCCCC5N5CCCCC5)nc4c3F)ccc(F)c2s1] | |
| 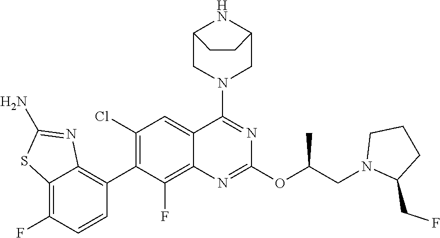 [SMILES: C[C@@H](CN1CCC[C@H]1CF)Oc1nc(N2CC3CCC(C2)N3)c2cc(Cl)c(-c3ccc(F)c4sc(N)nc34)c(F)c2n1] | |
| 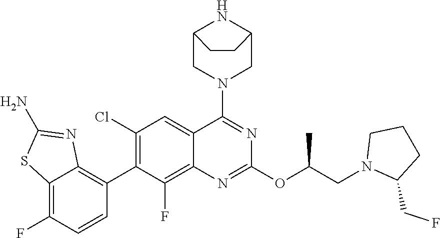 [SMILES: C[C@@H](CN1CCC[C@@H]1CF)Oc1nc(N2CC3CCC(C2)N3)c2cc(Cl)c(-c3ccc(F)c4sc(N)nc34)c(F)c2n1] | |
| 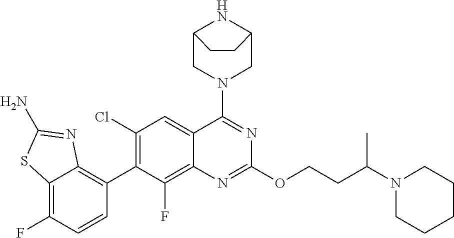 [SMILES: CC(CCOc1nc(N2CC3CCC(C2)N3)c2cc(Cl)c(-c3ccc(F)c4sc(N)nc34)c(F)c2n1)N1CCCCC1] | |
| 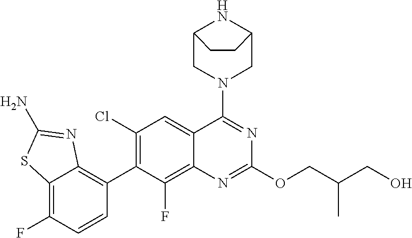 [SMILES: CC(CO)COc1nc(N2CC3CCC(C2)N3)c2cc(Cl)c(-c3ccc(F)c4sc(N)nc34)c(F)c2n1] | |
| 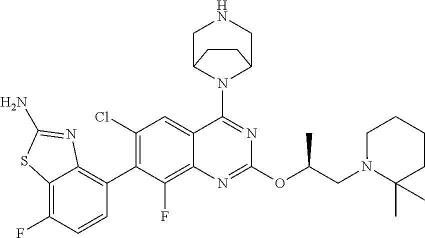 [SMILES: C[C@@H](CN1CCCCC1(C)C)Oc1nc(N2C3CCC2CNC3)c2cc(Cl)c(-c3ccc(F)c4sc(N)nc34)c(F)c2n1] | |
| 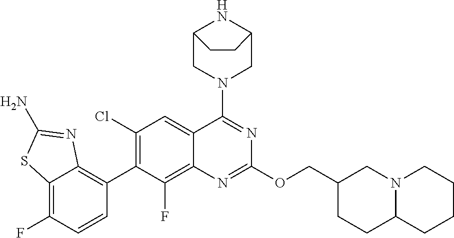 [SMILES: Nc1nc2c(-c3c(Cl)cc4c(N5CC6CCC(C5)N6)nc(OCC5CCC6CCCCN6C5)nc4c3F)ccc(F)c2s1] | |
| 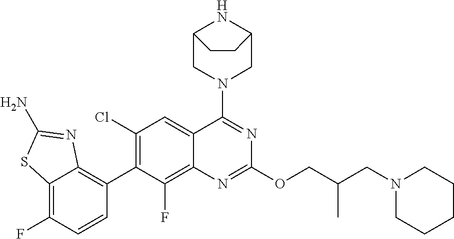 [SMILES: CC(COc1nc(N2CC3CCC(C2)N3)c2cc(Cl)c(-c3ccc(F)c4sc(N)nc34)c(F)c2n1)CN1CCCCC1] | |
| 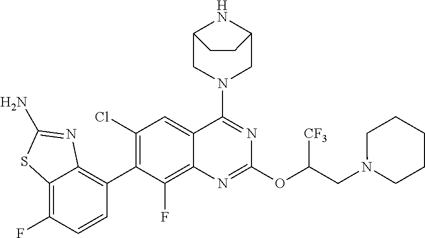 [SMILES: Nc1nc2c(-c3c(Cl)cc4c(N5CC6CCC(C5)N6)nc(OC(CN5CCCCC5)C(F)(F)F)nc4c3F)ccc(F)c2s1] | |
| 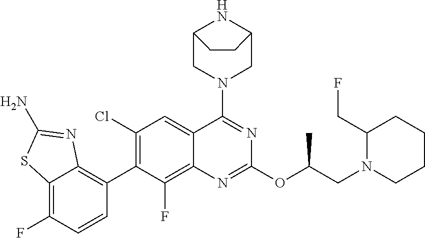 [SMILES: C[C@@H](CN1CCCCC1CF)Oc1nc(N2CC3CCC(C2)N3)c2cc(Cl)c(-c3ccc(F)c4sc(N)nc34)c(F)c2n1] | |
| 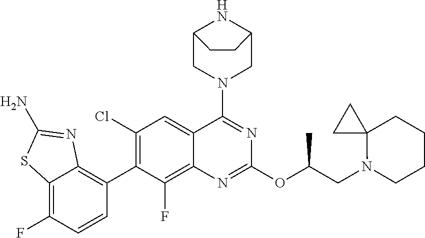 [SMILES: C[C@@H](CN1CCCCC12CC2)Oc1nc(N2CC3CCC(C2)N3)c2cc(Cl)c(-c3ccc(F)c4sc(N)nc34)c(F)c2n1] | |
| 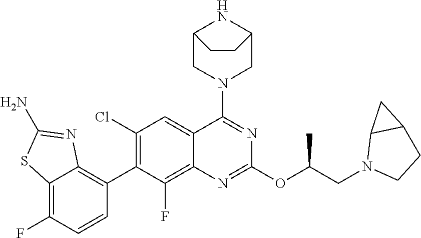 [SMILES: C[C@@H](CN1CCC2CC21)Oc1nc(N2CC3CCC(C2)N3)c2cc(Cl)c(-c3ccc(F)c4sc(N)nc34)c(F)c2n1] | |
| 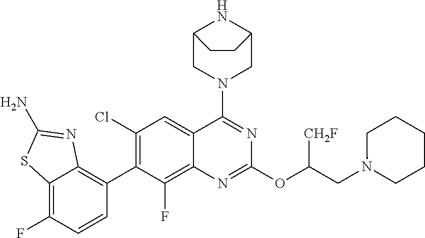 | |
| 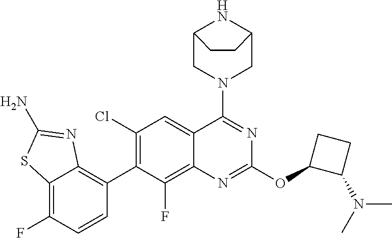 [SMILES: CN(C)[C@H]1CC[C@@H]1Oc1nc(N2CC3CCC(C2)N3)c2cc(Cl)c(-c3ccc(F)c4sc(N)nc34)c(F)c2n1] | |
| 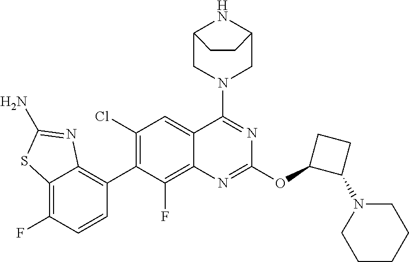 [SMILES: Nc1nc2c(-c3c(Cl)cc4c(N5CC6CCC(C5)N6)nc(O[C@H]5CC[C@@H]5N5CCCCC5)nc4c3F)ccc(F)c2s1] | |
| 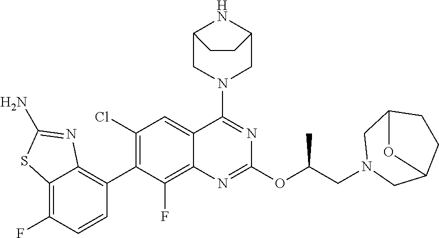 [SMILES: C[C@@H](CN1CC2CCC(C1)O2)Oc1nc(N2CC3CCC(C2)N3)c2cc(Cl)c(-c3ccc(F)c4sc(N)nc34)c(F)c2n1] | |
| 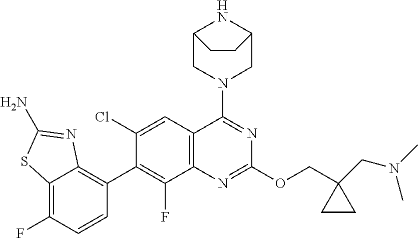 [SMILES: CN(C)CC1(COc2nc(N3CC4CCC(C3)N4)c3cc(Cl)c(-c4ccc(F)c5sc(N)nc45)c(F)c3n2)CC1] | |
| 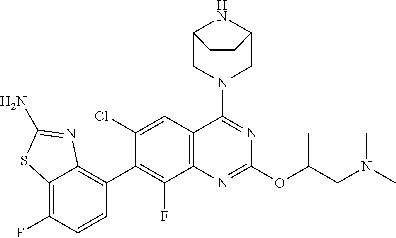 [SMILES: CC(CN(C)C)Oc1nc(N2CC3CCC(C2)N3)c2cc(Cl)c(-c3ccc(F)c4sc(N)nc34)c(F)c2n1] | |
| 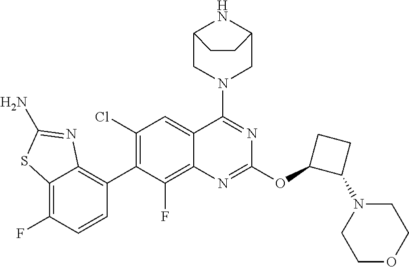 [SMILES: Nc1nc2c(-c3c(Cl)cc4c(N5CC6CCC(C5)N6)nc(O[C@H]5CC[C@@H]5N5CCOCC5)nc4c3F)ccc(F)c2s1] | |
| 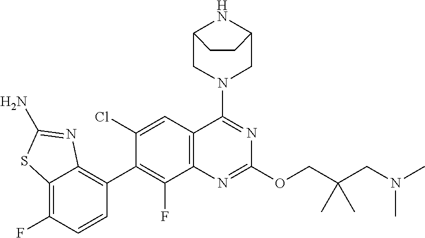 [SMILES: CN(C)CC(C)(C)COc1nc(N2CC3CCC(C2)N3)c2cc(Cl)c(-c3ccc(F)c4sc(N)nc34)c(F)c2n1] | |
| 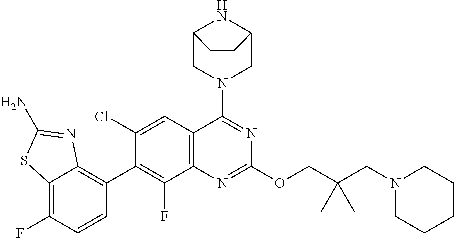 [SMILES: CC(C)(COc1nc(N2CC3CCC(C2)N3)c2cc(Cl)c(-c3ccc(F)c4sc(N)nc34)c(F)c2n1)CN1CCCCC1] | |
| 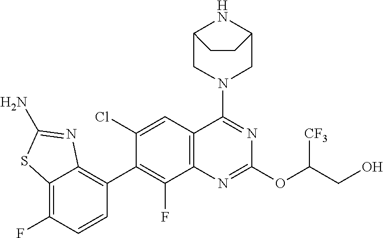 [SMILES: Nc1nc2c(-c3c(Cl)cc4c(N5CC6CCC(C5)N6)nc(OC(CO)C(F)(F)F)nc4c3F)ccc(F)c2s1] | |
| 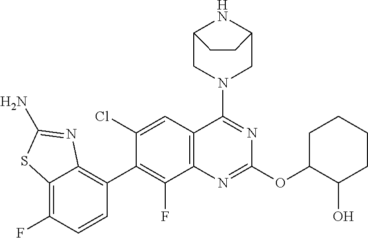 [SMILES: Nc1nc2c(-c3c(Cl)cc4c(N5CC6CCC(C5)N6)nc(OC5CCCCC5O)nc4c3F)ccc(F)c2s1] | |
| 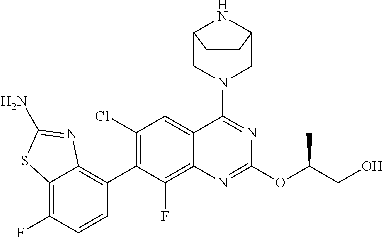 [SMILES: C[C@@H](CO)Oc1nc(N2CC3CCC(C2)N3)c2cc(Cl)c(-c3ccc(F)c4sc(N)nc34)c(F)c2n1] | |
| 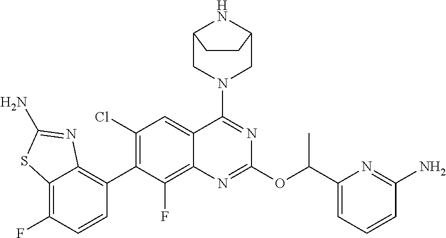 [SMILES: CC(Oc1nc(N2CC3CCC(C2)N3)c2cc(Cl)c(-c3ccc(F)c4sc(N)nc34)c(F)c2n1)c1cccc(N)n1] | |
| 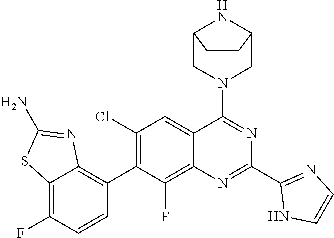 [SMILES: Nc1nc2c(-c3c(Cl)cc4c(N5CC6CCC(C5)N6)nc(-c5ncc[nH]5)nc4c3F)ccc(F)c2s1] | |
| 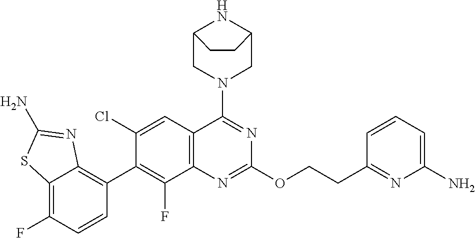 [SMILES: Nc1cccc(CCOc2nc(N3CC4CCC(C3)N4)c3cc(Cl)c(-c4ccc(F)c5sc(N)nc45)c(F)c3n2)n1] | |
| 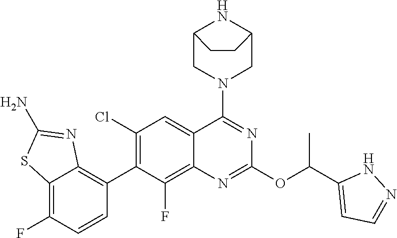 [SMILES: CC(Oc1nc(N2CC3CCC(C2)N3)c2cc(Cl)c(-c3ccc(F)c4sc(N)nc34)c(F)c2n1)c1ccn[nH]1] | |
| 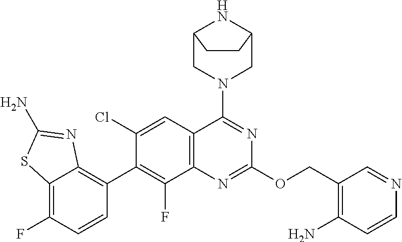 [SMILES: Nc1nc2c(-c3c(Cl)cc4c(N5CC6CCC(C5)N6)nc(OCc5cnccc5N)nc4c3F)ccc(F)c2s1] | |
| 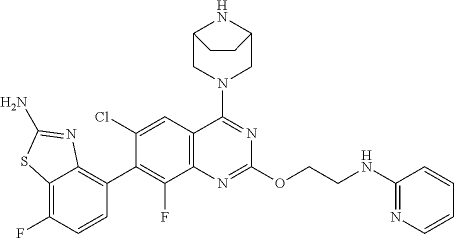 [SMILES: Nc1nc2c(-c3c(Cl)cc4c(N5CC6CCC(C5)N6)nc(OCCNc5ccccn5)nc4c3F)ccc(F)c2s1] | |
| 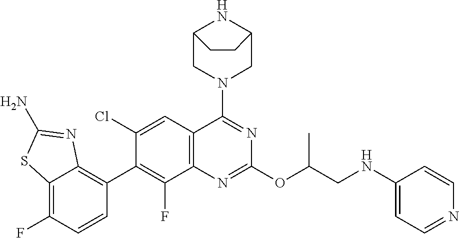 [SMILES: CC(CNc1ccncc1)Oc1nc(N2CC3CCC(C2)N3)c2cc(Cl)c(-c3ccc(F)c4sc(N)nc34)c(F)c2n1] | |
| 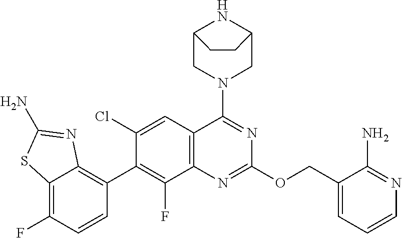 [SMILES: Nc1nc2c(-c3c(Cl)cc4c(N5CC6CCC(C5)N6)nc(OCc5cccnc5N)nc4c3F)ccc(F)c2s1] | |
| 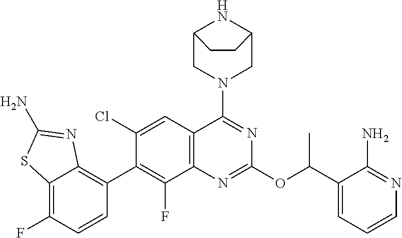 [SMILES: CC(Oc1nc(N2CC3CCC(C2)N3)c2cc(Cl)c(-c3ccc(F)c4sc(N)nc34)c(F)c2n1)c1cccnc1N] | |
| 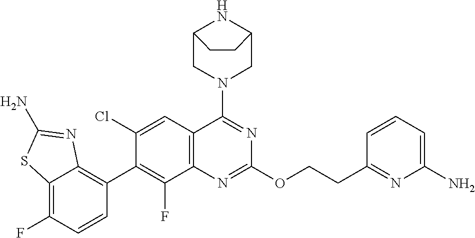 [SMILES: Nc1cccc(CCOc2nc(N3CC4CCC(C3)N4)c3cc(Cl)c(-c4ccc(F)c5sc(N)nc45)c(F)c3n2)n1] | |
| 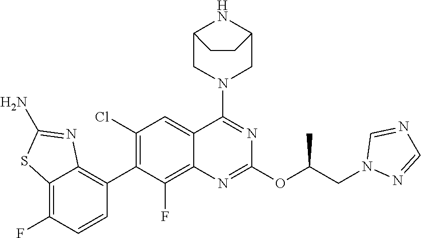 [SMILES: C[C@@H](Cn1cncn1)Oc1nc(N2CC3CCC(C2)N3)c2cc(Cl)c(-c3ccc(F)c4sc(N)nc34)c(F)c2n1] | |
| 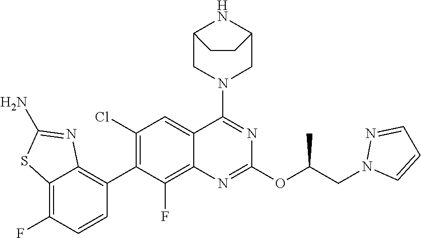 [SMILES: C[C@@H](Cn1cccn1)Oc1nc(N2CC3CCC(C2)N3)c2cc(Cl)c(-c3ccc(F)c4sc(N)nc34)c(F)c2n1] | |
| 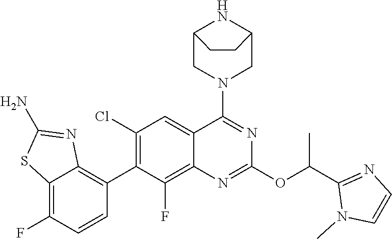 [SMILES: CC(Oc1nc(N2CC3CCC(C2)N3)c2cc(Cl)c(-c3ccc(F)c4sc(N)nc34)c(F)c2n1)c1nccn1C] | |
| 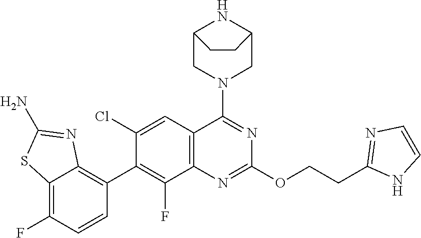 [SMILES: Nc1nc2c(-c3c(Cl)cc4c(N5CC6CCC(C5)N6)nc(OCCc5ncc[nH]5)nc4c3F)ccc(F)c2s1] | |
| 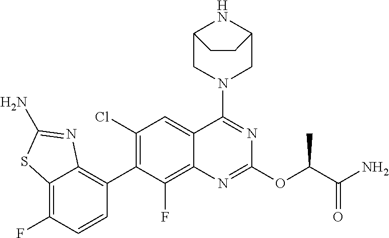 [SMILES: C[C@H](Oc1nc(N2CC3CCC(C2)N3)c2cc(Cl)c(-c3ccc(F)c4sc(N)nc34)c(F)c2n1)C(N)=O] | |
| 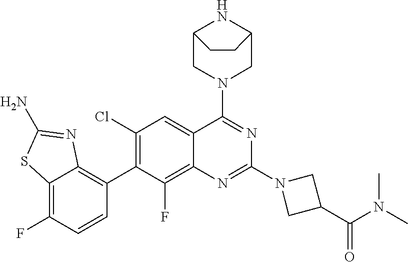 [SMILES: CN(C)C(=O)C1CN(c2nc(N3CC4CCC(C3)N4)c3cc(Cl)c(-c4ccc(F)c5sc(N)nc45)c(F)c3n2)C1] | |
| 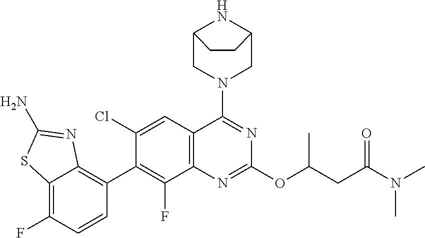 [SMILES: CC(CC(=O)N(C)C)Oc1nc(N2CC3CCC(C2)N3)c2cc(Cl)c(-c3ccc(F)c4sc(N)nc34)c(F)c2n1] | |
| 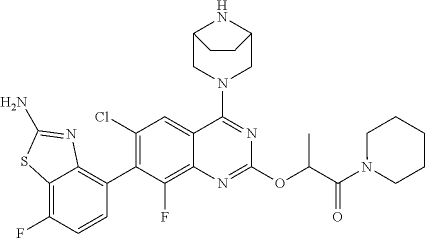 [SMILES: CC(Oc1nc(N2CC3CCC(C2)N3)c2cc(Cl)c(-c3ccc(F)c4sc(N)nc34)c(F)c2n1)C(=O)N1CCCCC1] | |
| 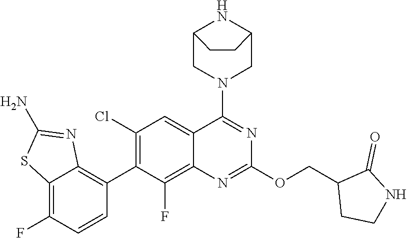 [SMILES: Nc1nc2c(-c3c(Cl)cc4c(N5CC6CCC(C5)N6)nc(OCC5CCNC5=O)nc4c3F)ccc(F)c2s1] | |
| 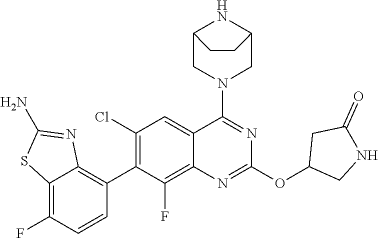 [SMILES: Nc1nc2c(-c3c(Cl)cc4c(N5CC6CCC(C5)N6)nc(OC5CNC(=O)C5)nc4c3F)ccc(F)c2s1] | |
| 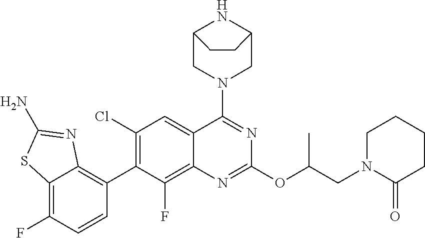 [SMILES: CC(CN1CCCCC1=O)Oc1nc(N2CC3CCC(C2)N3)c2cc(Cl)c(-c3ccc(F)c4sc(N)nc34)c(F)c2n1] | |
| 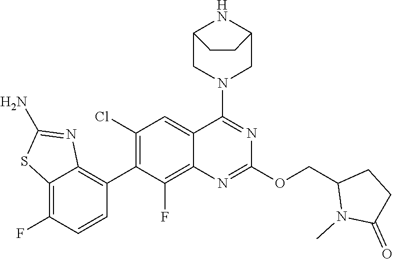 [SMILES: CN1C(=O)CCC1COc1nc(N2CC3CCC(C2)N3)c2cc(Cl)c(-c3ccc(F)c4sc(N)nc34)c(F)c2n1] | |
| 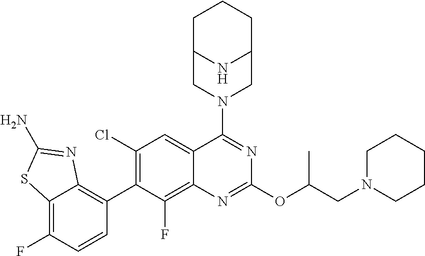 [SMILES: CC(CN1CCCCC1)Oc1nc(N2CC3CCCC(C2)N3)c2cc(Cl)c(-c3ccc(F)c4sc(N)nc34)c(F)c2n1] | |
| 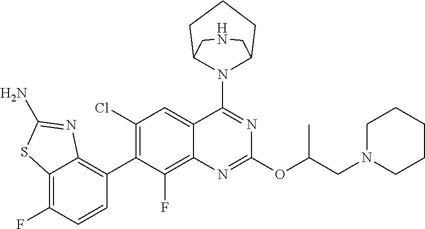 [SMILES: CC(CN1CCCCC1)Oc1nc(N2C3CCCC2CNC3)c2cc(Cl)c(-c3ccc(F)c4sc(N)nc34)c(F)c2n1] | |
| 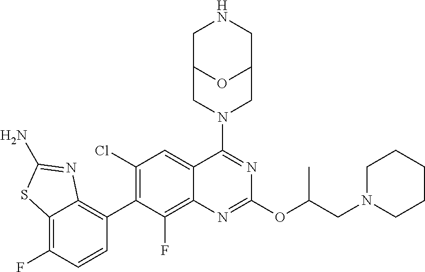 [SMILES: CC(CN1CCCCC1)Oc1nc(N2CC3CNCC(C2)O3)c2cc(Cl)c(-c3ccc(F)c4sc(N)nc34)c(F)c2n1] | |
| 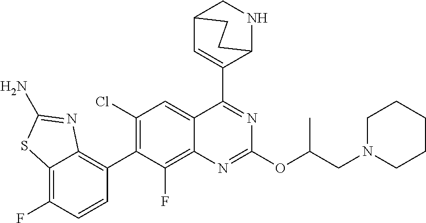 [SMILES: CC(CN1CCCCC1)Oc1nc(C2=CC3CCC2NC3)c2cc(Cl)c(-c3ccc(F)c4sc(N)nc34)c(F)c2n1] | |
| 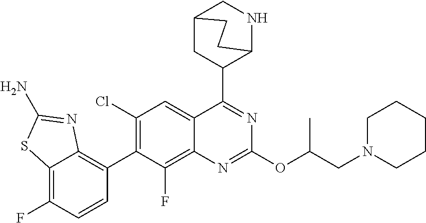 [SMILES: CC(CN1CCCCC1)Oc1nc(C2CC3CCC2NC3)c2cc(Cl)c(-c3ccc(F)c4sc(N)nc34)c(F)c2n1] | |
| 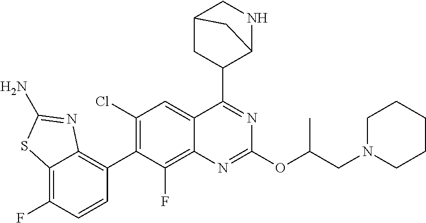 [SMILES: CC(CN1CCCCC1)Oc1nc(C2CC3CNC2C3)c2cc(Cl)c(-c3ccc(F)c4sc(N)nc34)c(F)c2n1] | |
| 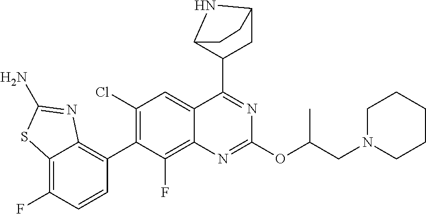 [SMILES: CC(CN1CCCCC1)Oc1nc(C2CC3CCC2N3)c2cc(Cl)c(-c3ccc(F)c4sc(N)nc34)c(F)c2n1] | |
| 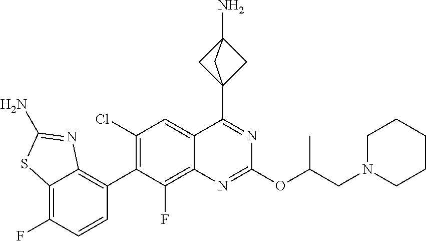 [SMILES: CC(CN1CCCCC1)Oc1nc(C23CC(N)(C2)C3)c2cc(Cl)c(-c3ccc(F)c4sc(N)nc34)c(F)c2n1] | |
| 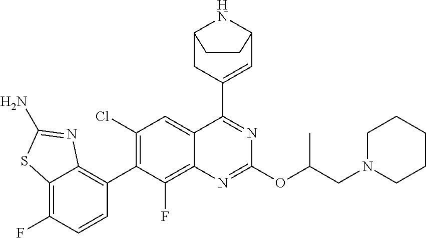 [SMILES: CC(CN1CCCCC1)Oc1nc(C2=CC3CCC(C2)N3)c2cc(Cl)c(-c3ccc(F)c4sc(N)nc34)c(F)c2n1] | |
| 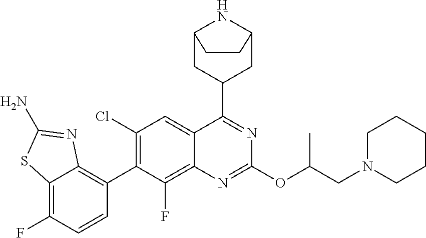 [SMILES: CC(CN1CCCCC1)Oc1nc(C2CC3CCC(C2)N3)c2cc(Cl)c(-c3ccc(F)c4sc(N)nc34)c(F)c2n1] | |
| 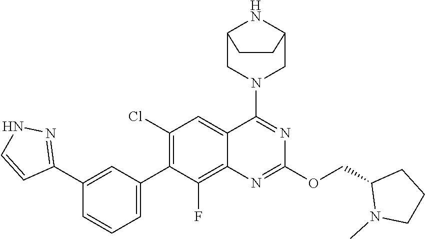 [SMILES: CN1CCC[C@H]1COc1nc(N2CC3CCC(C2)N3)c2cc(Cl)c(-c3cccc(-c4cc[nH]n4)c3)c(F)c2n1] | |
| 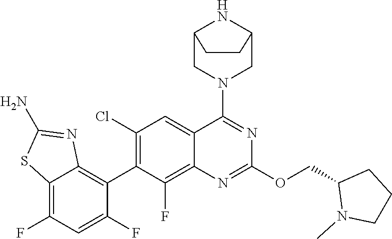 [SMILES: CN1CCC[C@H]1COc1nc(N2CC3CCC(C2)N3)c2cc(Cl)c(-c3c(F)cc(F)c4sc(N)nc34)c(F)c2n1] | |
| 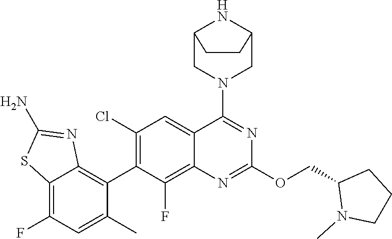 [SMILES: Cc1cc(F)c2sc(N)nc2c1-c1c(Cl)cc2c(N3CC4CCC(C3)N4)nc(OC[C@@H]3CCCN3C)nc2c1F] | |
| 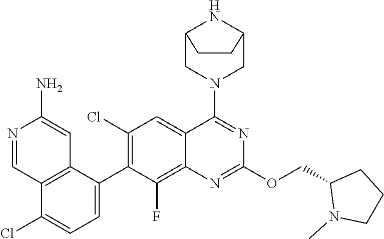 [SMILES: CN1CCC[C@H]1COc1nc(N2CC3CCC(C2)N3)c2cc(Cl)c(-c3ccc(Cl)c4cnc(N)cc34)c(F)c2n1] | |
| 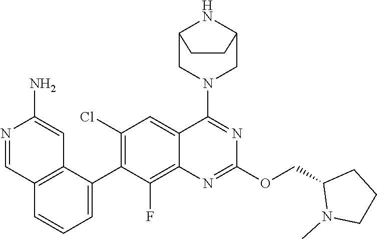 [SMILES: CN1CCC[C@H]1COc1nc(N2CC3CCC(C2)N3)c2cc(Cl)c(-c3cccc4cnc(N)cc34)c(F)c2n1] | |
| 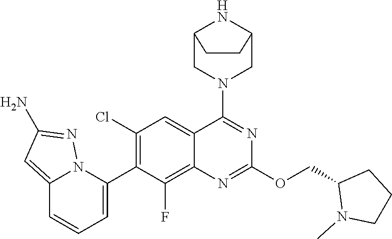 [SMILES: CN1CCC[C@H]1COc1nc(N2CC3CCC(C2)N3)c2cc(Cl)c(-c3cccc4cc(N)nn34)c(F)c2n1] | |
| 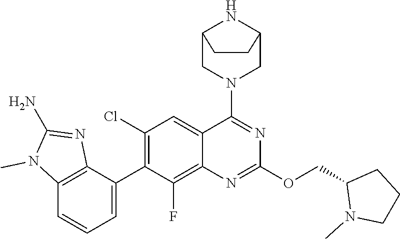 [SMILES: CN1CCC[C@H]1COc1nc(N2CC3CCC(C2)N3)c2cc(Cl)c(-c3cccc4c3nc(N)n4C)c(F)c2n1] | |
| 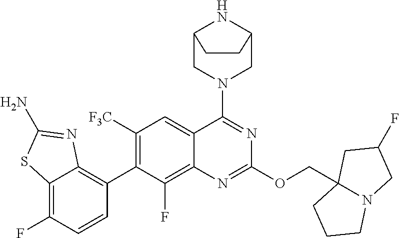 [SMILES: Nc1nc2c(-c3c(C(F)(F)F)cc4c(N5CC6CCC(C5)N6)nc(OCC56CCCN5CC(F)C6)nc4c3F)ccc(F)c2s1] | |
| 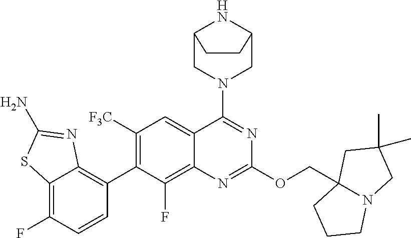 [SMILES: CC1(C)CN2CCCC2(COc2nc(N3CC4CCC(C3)N4)c3cc(C(F)(F)F)c(-c4ccc(F)c5sc(N)nc45)c(F)c3n2)C1] | |
| 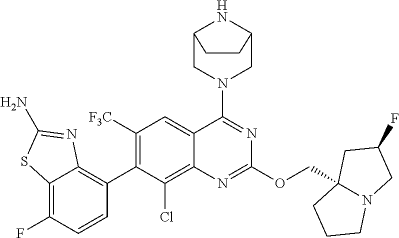 [SMILES: Nc1nc2c(-c3c(C(F)(F)F)cc4c(N5CC6CCC(C5)N6)nc(OC[C@@]56CCCN5C[C@H](F)C6)nc4c3Cl)ccc(F)c2s1] | |
| 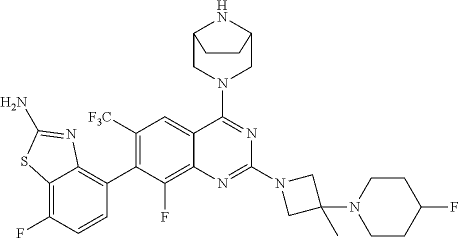 [SMILES: CC1(N2CCC(F)CC2)CN(c2nc(N3CC4CCC(C3)N4)c3cc(C(F)(F)F)c(-c4ccc(F)c5sc(N)nc45)c(F)c3n2)C1] | |
| 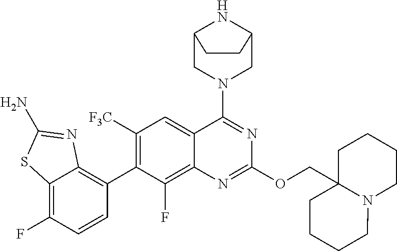 [SMILES: Nc1nc2c(-c3c(C(F)(F)F)cc4c(N5CC6CCC(C5)N6)nc(OCC56CCCCN5CCCC6)nc4c3F)ccc(F)c2s1] | |
| 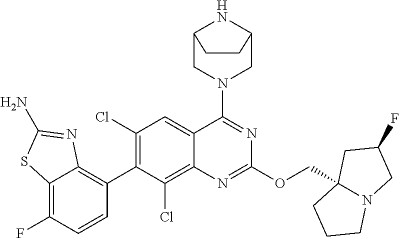 [SMILES: Nc1nc2c(-c3c(Cl)cc4c(N5CC6CCC(C5)N6)nc(OC[C@@]56CCCN5C[C@H](F)C6)nc4c3Cl)ccc(F)c2s1] | |
| 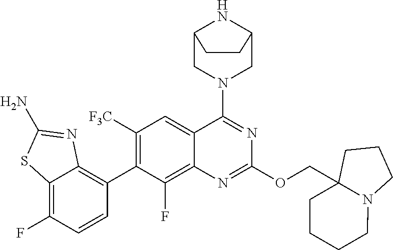 [SMILES: Nc1nc2c(-c3c(C(F)(F)F)cc4c(N5CC6CCC(C5)N6)nc(OCC56CCCCN5CCC6)nc4c3F)ccc(F)c2s1] | |
| 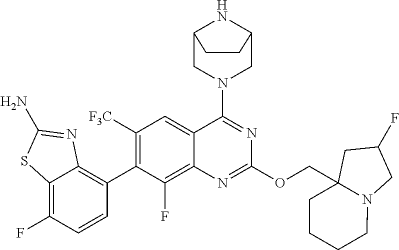 [SMILES: Nc1nc2c(-c3c(C(F)(F)F)cc4c(N5CC6CCC(C5)N6)nc(OCC56CCCCN5CC(F)C6)nc4c3F)ccc(F)c2s1] | |
| 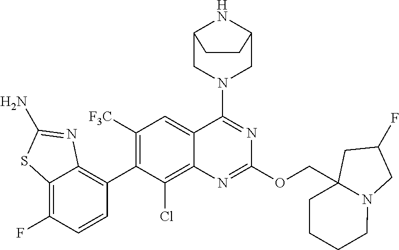 [SMILES: Nc1nc2c(-c3c(C(F)(F)F)cc4c(N5CC6CCC(C5)N6)nc(OCC56CCCCN5CC(F)C6)nc4c3Cl)ccc(F)c2s1] | |
| 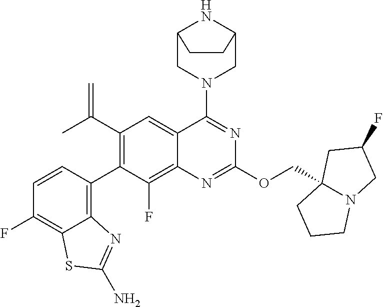 [SMILES: C=C(C)c1cc2c(N3CC4CCC(C3)N4)nc(OC[C@@]34CCCN3C[C@H](F)C4)nc2c(F)c1-c1ccc(F)c2sc(N)nc12] | |
| 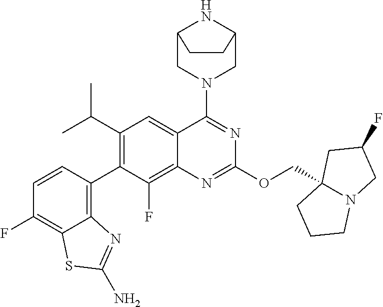 [SMILES: CC(C)c1cc2c(N3CC4CCC(C3)N4)nc(OC[C@@]34CCCN3C[C@H](F)C4)nc2c(F)c1-c1ccc(F)c2sc(N)nc12] | |
| 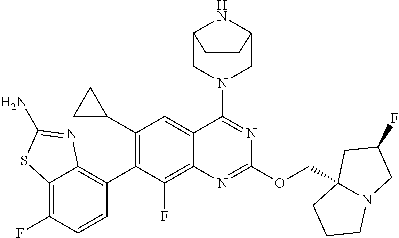 [SMILES: Nc1nc2c(-c3c(C4CC4)cc4c(N5CC6CCC(C5)N6)nc(OC[C@@]56CCCN5C[C@H](F)C6)nc4c3F)ccc(F)c2s1] | |
| 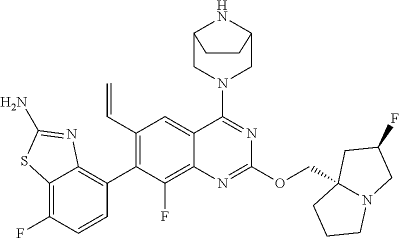 [SMILES: C=Cc1cc2c(N3CC4CCC(C3)N4)nc(OC[C@@]34CCCN3C[C@H](F)C4)nc2c(F)c1-c1ccc(F)c2sc(N)nc12] | |
| 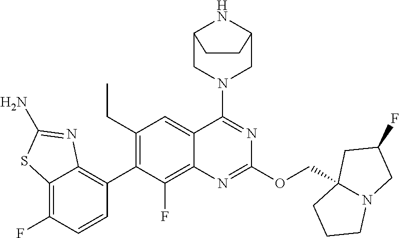 [SMILES: CCc1cc2c(N3CC4CCC(C3)N4)nc(OC[C@@]34CCCN3C[C@H](F)C4)nc2c(F)c1-c1ccc(F)c2sc(N)nc12] | |
| 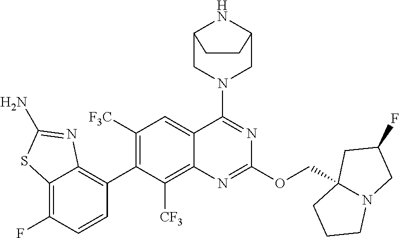 [SMILES: Nc1nc2c(-c3c(C(F)(F)F)cc4c(N5CC6CCC(C5)N6)nc(OC[C@@]56CCCN5C[C@H](F)C6)nc4c3C(F)(F)F)ccc(F)c2s1] | |
| 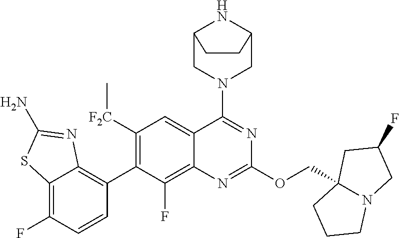 [SMILES: CC(F)(F)c1cc2c(N3CC4CCC(C3)N4)nc(OC[C@@]34CCCN3C[C@H](F)C4)nc2c(F)c1-c1ccc(F)c2sc(N)nc12] | |
| 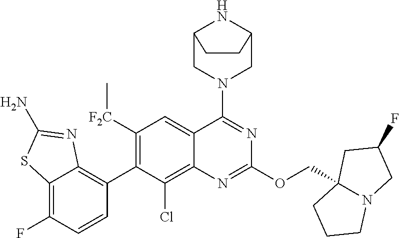 [SMILES: CC(F)(F)c1cc2c(N3CC4CCC(C3)N4)nc(OC[C@@]34CCCN3C[C@H](F)C4)nc2c(Cl)c1-c1ccc(F)c2sc(N)nc12] | |
| 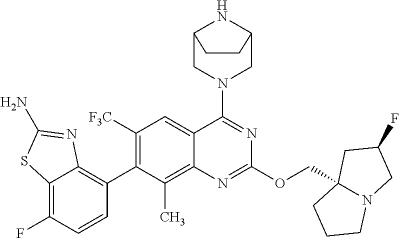 [SMILES: Cc1c(-c2ccc(F)c3sc(N)nc23)c(C(F)(F)F)cc2c(N3CC4CCC(C3)N4)nc(OC[C@@]34CCCN3C[C@H](F)C4)nc12] | |
| 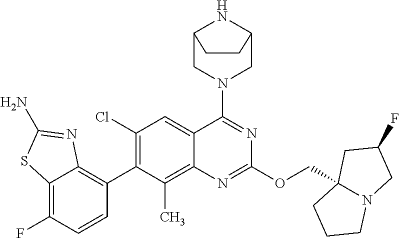 [SMILES: Cc1c(-c2ccc(F)c3sc(N)nc23)c(Cl)cc2c(N3CC4CCC(C3)N4)nc(OC[C@@]34CCCN3C[C@H](F)C4)nc12] | |
| 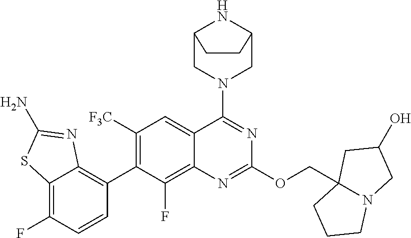 [SMILES: Nc1nc2c(-c3c(C(F)(F)F)cc4c(N5CC6CCC(C5)N6)nc(OCC56CCCN5CC(O)C6)nc4c3F)ccc(F)c2s1] | |
| 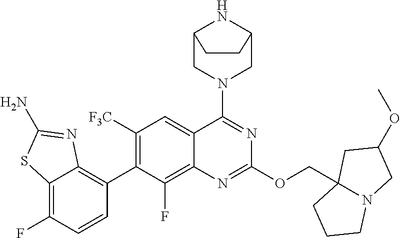 [SMILES: COC1CN2CCCC2(COc2nc(N3CC4CCC(C3)N4)c3cc(C(F)(F)F)c(-c4ccc(F)c5sc(N)nc45)c(F)c3n2)C1] | |
| 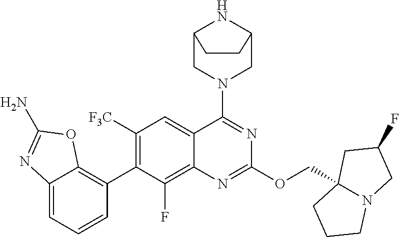 [SMILES: Nc1nc2cccc(-c3c(C(F)(F)F)cc4c(N5CC6CCC(C5)N6)nc(OC[C@@]56CCCN5C[C@H](F)C6)nc4c3F)c2o1] | |
| 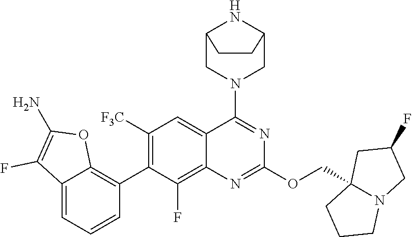 [SMILES: Nc1oc2c(-c3c(C(F)(F)F)cc4c(N5CC6CCC(C5)N6)nc(OC[C@@]56CCCN5C[C@H](F)C6)nc4c3F)cccc2c1F] | |
| 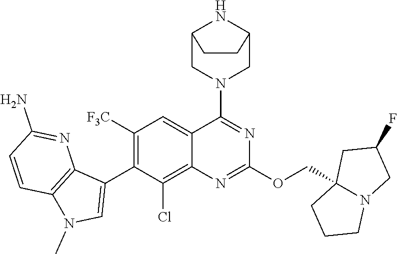 [SMILES: Cn1cc(-c2c(C(F)(F)F)cc3c(N4CC5CCC(C4)N5)nc(OC[C@@]45CCCN4C[C@H](F)C5)nc3c2Cl)c2nc(N)ccc21] | |
| 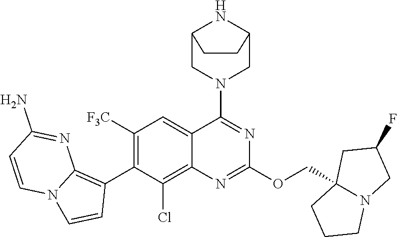 [SMILES: Nc1ccn2ccc(-c3c(C(F)(F)F)cc4c(N5CC6CCC(C5)N6)nc(OC[C@@]56CCCN5C[C@H](F)C6)nc4c3Cl)c2n1] | |
| 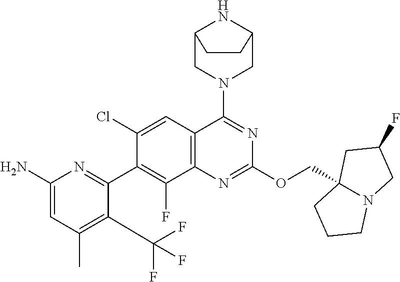 [SMILES: Cc1cc(N)nc(-c2c(Cl)cc3c(N4CC5CCC(C4)N5)nc(OC[C@@]45CCCN4C[C@H](F)C5)nc3c2F)c1C(F)(F)F] | |
| 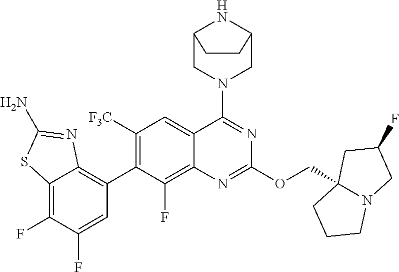 [SMILES: Nc1nc2c(-c3c(C(F)(F)F)cc4c(N5CC6CCC(C5)N6)nc(OC[C@@]56CCCN5C[C@H](F)C6)nc4c3F)cc(F)c(F)c2s1] | |
| 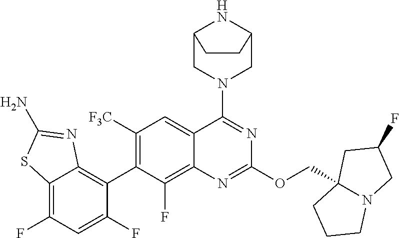 [SMILES: Nc1nc2c(-c3c(C(F)(F)F)cc4c(N5CC6CCC(C5)N6)nc(OC[C@@]56CCCN5C[C@H](F)C6)nc4c3F)c(F)cc(F)c2s1] | |
| 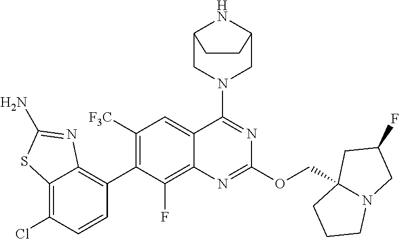 [SMILES: Nc1nc2c(-c3c(C(F)(F)F)cc4c(N5CC6CCC(C5)N6)nc(OC[C@@]56CCCN5C[C@H](F)C6)nc4c3F)ccc(Cl)c2s1] | |
| 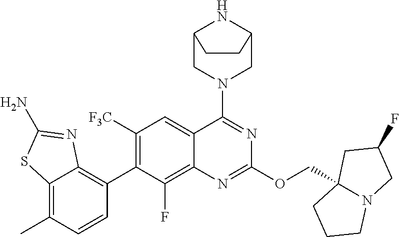 [SMILES: Cc1ccc(-c2c(C(F)(F)F)cc3c(N4CC5CCC(C4)N5)nc(OC[C@@]45CCCN4C[C@H](F)C5)nc3c2F)c2nc(N)sc12] | |
| 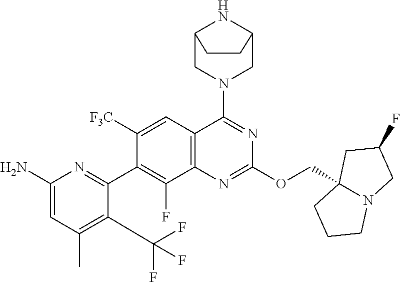 [SMILES: Cc1cc(N)nc(-c2c(C(F)(F)F)cc3c(N4CC5CCC(C4)N5)nc(OC[C@@]45CCCN4C[C@H](F)C5)nc3c2F)c1C(F)(F)F] | |
| 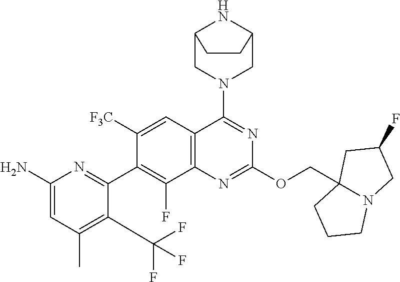 [SMILES: Cc1cc(N)nc(-c2c(C(F)(F)F)cc3c(N4CC5CCC(C4)N5)nc(OCC45CCCN4C[C@H](F)C5)nc3c2F)c1C(F)(F)F] | |
| 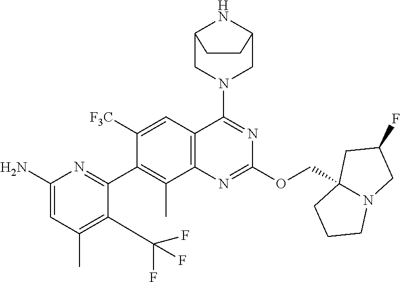 [SMILES: Cc1cc(N)nc(-c2c(C(F)(F)F)cc3c(N4CC5CCC(C4)N5)nc(OC[C@@]45CCCN4C[C@H](F)C5)nc3c2C)c1C(F)(F)F] | |
| 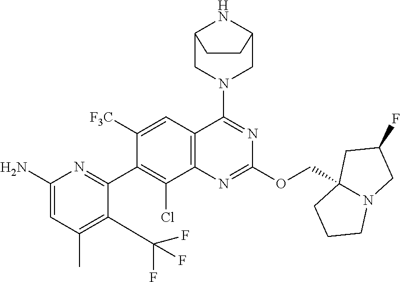 [SMILES: Cc1cc(N)nc(-c2c(C(F)(F)F)cc3c(N4CC5CCC(C4)N5)nc(OC[C@@]45CCCN4C[C@H](F)C5)nc3c2Cl)c1C(F)(F)F] | |
| 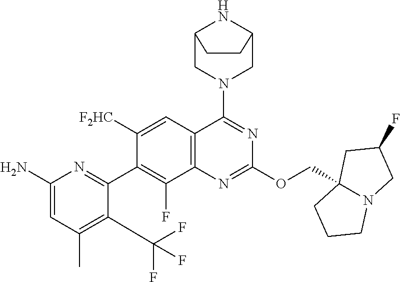 [SMILES: Cc1cc(N)nc(-c2c(C(F)F)cc3c(N4CC5CCC(C4)N5)nc(OC[C@@]45CCCN4C[C@H](F)C5)nc3c2F)c1C(F)(F)F] | |
| 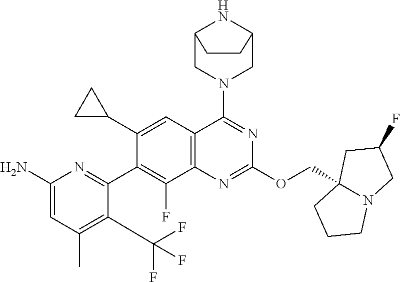 [SMILES: Cc1cc(N)nc(-c2c(C3CC3)cc3c(N4CC5CCC(C4)N5)nc(OC[C@@]45CCCN4C[C@H](F)C5)nc3c2F)c1C(F)(F)F] | |
| 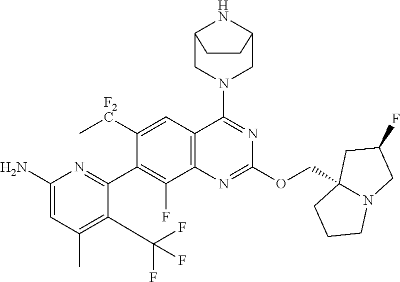 [SMILES: Cc1cc(N)nc(-c2c(C(C)(F)F)cc3c(N4CC5CCC(C4)N5)nc(OC[C@@]45CCCN4C[C@H](F)C5)nc3c2F)c1C(F)(F)F] | |
| 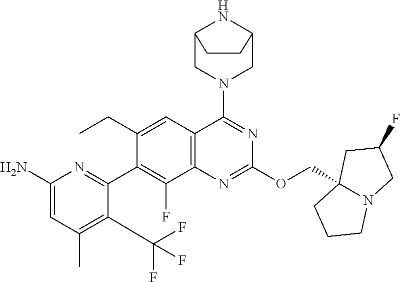 [SMILES: CCc1cc2c(N3CC4CCC(C3)N4)nc(OC[C@@]34CCCN3C[C@H](F)C4)nc2c(F)c1-c1nc(N)cc(C)c1C(F)(F)F] | |
| 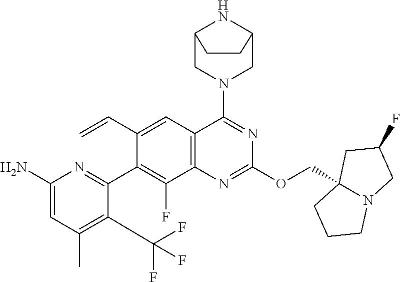 [SMILES: C=Cc1cc2c(N3CC4CCC(C3)N4)nc(OC[C@@]34CCCN3C[C@H](F)C4)nc2c(F)c1-c1nc(N)cc(C)c1C(F)(F)F] | |
| 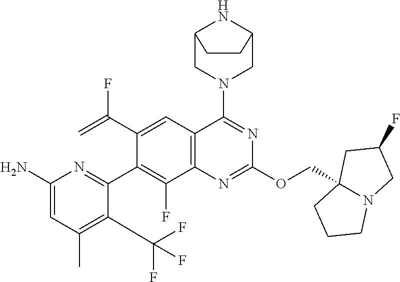 [SMILES: C=C(F)c1cc2c(N3CC4CCC(C3)N4)nc(OC[C@@]34CCCN3C[C@H](F)C4)nc2c(F)c1-c1nc(N)cc(C)c1C(F)(F)F] | |
| 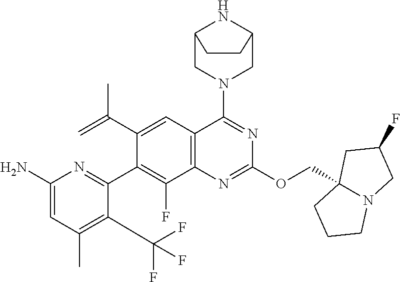 [SMILES: C=C(C)c1cc2c(N3CC4CCC(C3)N4)nc(OC[C@@]34CCCN3C[C@H](F)C4)nc2c(F)c1-c1nc(N)cc(C)c1C(F)(F)F] | |
| 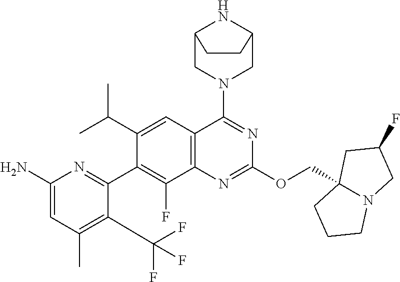 [SMILES: Cc1cc(N)nc(-c2c(C(C)C)cc3c(N4CC5CCC(C4)N5)nc(OC[C@@]45CCCN4C[C@H](F)C5)nc3c2F)c1C(F)(F)F] | |
| 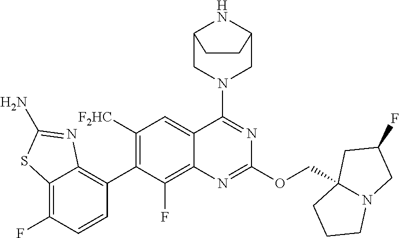 [SMILES: Nc1nc2c(-c3c(C(F)F)cc4c(N5CC6CCC(C5)N6)nc(OC[C@@]56CCCN5C[C@H](F)C6)nc4c3F)ccc(F)c2s1] | |
| 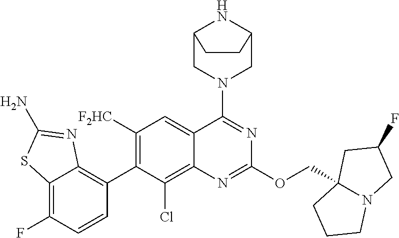 [SMILES: Nc1nc2c(-c3c(C(F)F)cc4c(N5CC6CCC(C5)N6)nc(OC[C@@]56CCCN5C[C@H](F)C6)nc4c3Cl)ccc(F)c2s1] | |
| 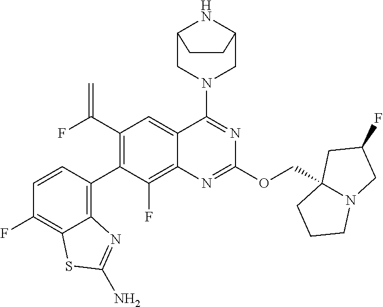 [SMILES: C=C(F)c1cc2c(N3CC4CCC(C3)N4)nc(OC[C@@]34CCCN3C[C@H](F)C4)nc2c(F)c1-c1ccc(F)c2sc(N)nc12] | |
| 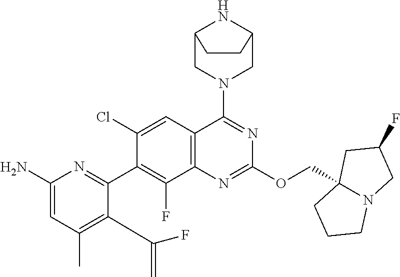 [SMILES: C=C(F)c1c(C)cc(N)nc1-c1c(Cl)cc2c(N3CC4CCC(C3)N4)nc(OC[C@@]34CCCN3C[C@H](F)C4)nc2c1F] | |
| 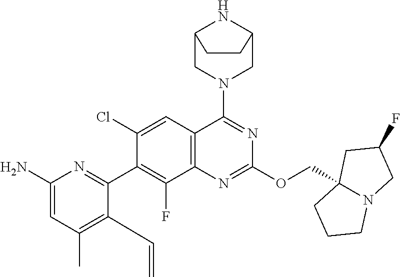 [SMILES: C=Cc1c(C)cc(N)nc1-c1c(Cl)cc2c(N3CC4CCC(C3)N4)nc(OC[C@@]34CCCN3C[C@H](F)C4)nc2c1F] | |
| 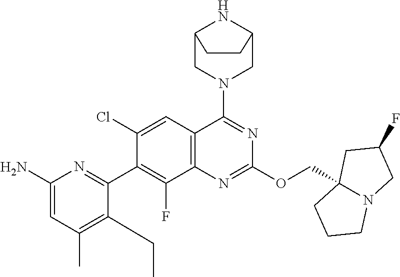 [SMILES: CCc1c(C)cc(N)nc1-c1c(Cl)cc2c(N3CC4CCC(C3)N4)nc(OC[C@@]34CCCN3C[C@H](F)C4)nc2c1F] | |
| 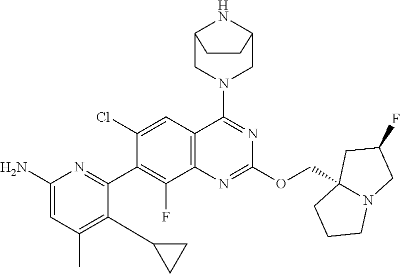 [SMILES: Cc1cc(N)nc(-c2c(Cl)cc3c(N4CC5CCC(C4)N5)nc(OC[C@@]45CCCN4C[C@H](F)C5)nc3c2F)c1C1CC1] | |
| 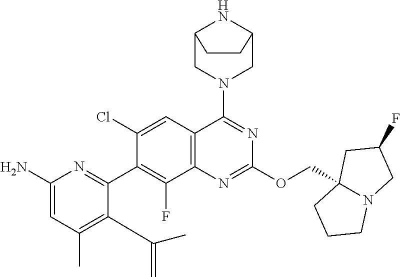 [SMILES: C=C(C)c1c(C)cc(N)nc1-c1c(Cl)cc2c(N3CC4CCC(C3)N4)nc(OC[C@@]34CCCN3C[C@H](F)C4)nc2c1F] | |
| 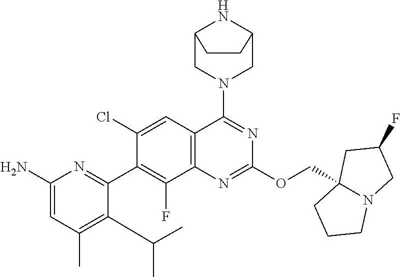 [SMILES: Cc1cc(N)nc(-c2c(Cl)cc3c(N4CC5CCC(C4)N5)nc(OC[C@@]45CCCN4C[C@H](F)C5)nc3c2F)c1C(C)C] | |
| 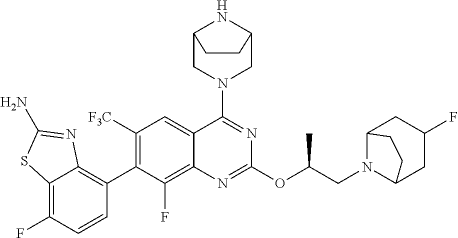 [SMILES: C[C@@H](CN1C2CCC1CC(F)C2)Oc1nc(N2CC3CCC(C2)N3)c2cc(C(F)(F)F)c(-c3ccc(F)c4sc(N)nc34)c(F)c2n1] | |
| 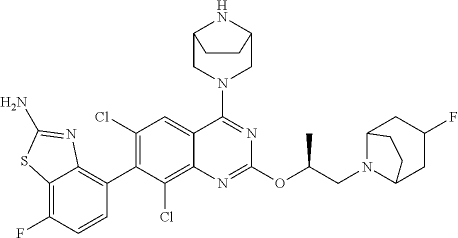 [SMILES: C[C@@H](CN1C2CCC1CC(F)C2)Oc1nc(N2CC3CCC(C2)N3)c2cc(Cl)c(-c3ccc(F)c4sc(N)nc34)c(Cl)c2n1] | |
| 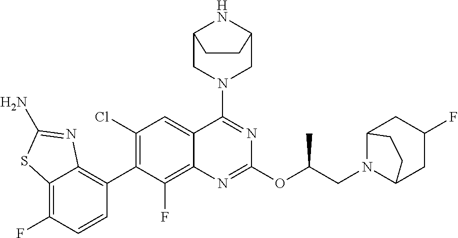 [SMILES: C[C@@H](CN1C2CCC1CC(F)C2)Oc1nc(N2CC3CCC(C2)N3)c2cc(Cl)c(-c3ccc(F)c4sc(N)nc34)c(F)c2n1] | |
| 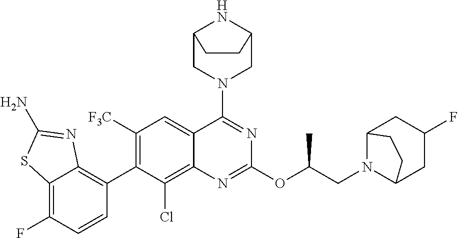 [SMILES: C[C@@H](CN1C2CCC1CC(F)C2)Oc1nc(N2CC3CCC(C2)N3)c2cc(C(F)(F)F)c(-c3ccc(F)c4sc(N)nc34)c(Cl)c2n1] | |
| 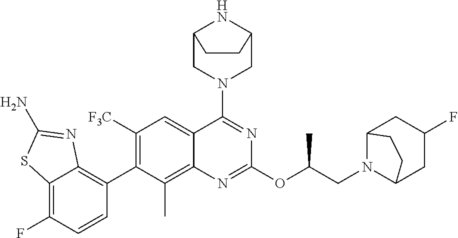 [SMILES: Cc1c(-c2ccc(F)c3sc(N)nc23)c(C(F)(F)F)cc2c(N3CC4CCC(C3)N4)nc(O[C@@H](C)CN3C4CCC3CC(F)C4)nc12] | |
| 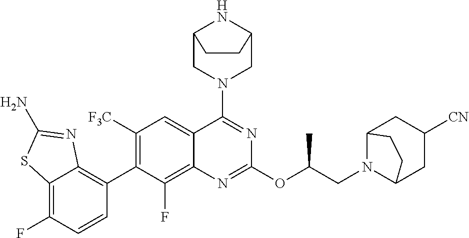 [SMILES: C[C@@H](CN1C2CCC1CC(C#N)C2)Oc1nc(N2CC3CCC(C2)N3)c2cc(C(F)(F)F)c(-c3ccc(F)c4sc(N)nc34)c(F)c2n1] | |
| 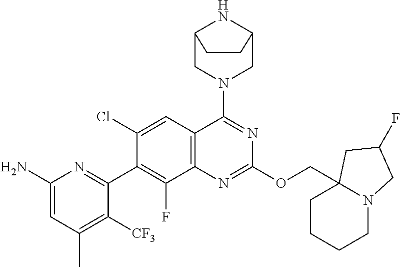 [SMILES: Cc1cc(N)nc(-c2c(Cl)cc3c(N4CC5CCC(C4)N5)nc(OCC45CCCCN4CC(F)C5)nc3c2F)c1C(F)(F)F] | |
| 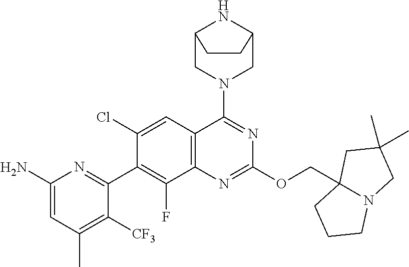 [SMILES: Cc1cc(N)nc(-c2c(Cl)cc3c(N4CC5CCC(C4)N5)nc(OCC45CCCN4CC(C)(C)C5)nc3c2F)c1C(F)(F)F] | |
| 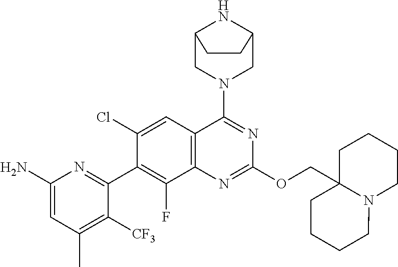 [SMILES: Cc1cc(N)nc(-c2c(Cl)cc3c(N4CC5CCC(C4)N5)nc(OCC45CCCCN4CCCC5)nc3c2F)c1C(F)(F)F] | |
| 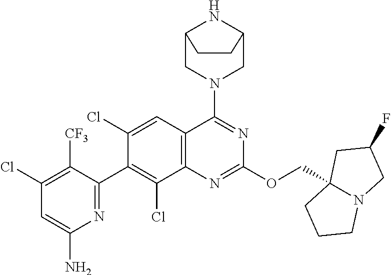 [SMILES: Nc1cc(Cl)c(C(F)(F)F)c(-c2c(Cl)cc3c(N4CC5CCC(C4)N5)nc(OC[C@@]45CCCN4C[C@H](F)C5)nc3c2Cl)n1] | |
| 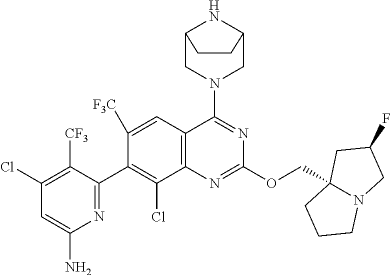 [SMILES: Nc1cc(Cl)c(C(F)(F)F)c(-c2c(C(F)(F)F)cc3c(N4CC5CCC(C4)N5)nc(OC[C@@]45CCCN4C[C@H](F)C5)nc3c2Cl)n1] | |

Aspect 38a: In some embodiments, representative compounds of formulas (I) and (VIII) are set forth in the following table:

TABLE 2

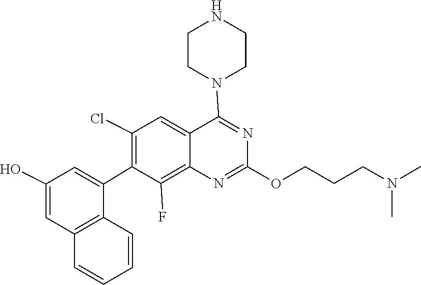 [SMILES: CN(C)CCCOc1nc(N2CCNCC2)c2cc(Cl)c(-c3cc(O)cc4ccccc34)c(F)c2n1]

E1: 4-(6-chloro-2-(3-
(dimethylamino)propoxy)-8-
fluoro-4-(piperazin-1-
yl)quinazolin-7-yl)naphthalen-2-
ol

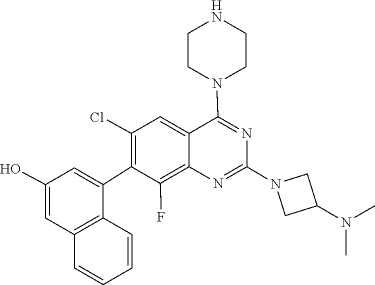 [SMILES: CN(C)C1CN(c2nc(N3CCNCC3)c3cc(Cl)c(-c4cc(O)cc5ccccc45)c(F)c3n2)C1]

E2: 4-(6-chloro-2-(3-
(dimethylamino)azetidin-1-
yl)-8-fluoro-4-(piperazin-1-
yl)quinazolin-7-yl)naphthalen-
2-ol

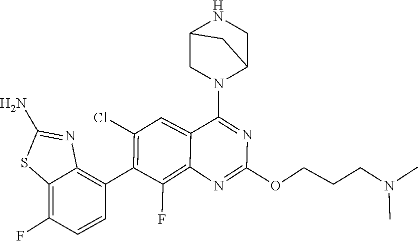 [SMILES: CN(C)CCCOc1nc(N2CC3CC2CN3)c2cc(Cl)c(-c3ccc(F)c4sc(N)nc34)c(F)c2n1]

E3: 4-(4-(2,5-
diazabicyclo[2.2.1]heptan-2-yl)-
6-chloro-2-(3-
(dimethylamino)propoxy)-8-
fluoroquinazolin-7-yl)-7-
fluorobenzo[d]thiazol-2-amine

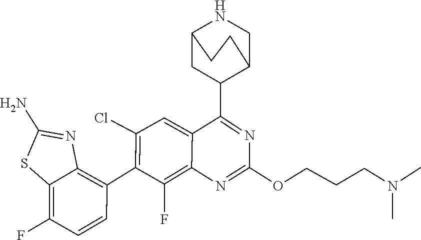 [SMILES: CN(C)CCCOc1nc(C2CC3CCC2CN3)c2cc(Cl)c(-c3ccc(F)c4sc(N)nc34)c(F)c2n1]

E4: 4-(4-(2,5-
diazabicyclo[2.2.2]octan-2-yl)-6-
chloro-2-(3-
(dimethylamino)propoxy)-8-
fluoroquinazolin-7-yl)-7-
fluorobenzo[d]thiazol-2-amine

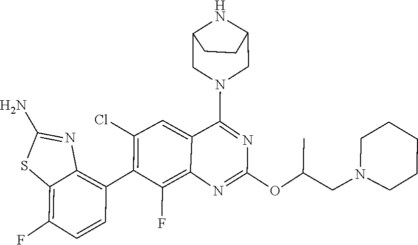 [SMILES: CC(CN1CCCCC1)Oc1nc(N2CC3CCC(C2)N3)c2cc(Cl)c(-c3ccc(F)c4sc(N)nc34)c(F)c2n1]

E5: 4-(4-(3,8-
diazabicyclo[3.2.1]octan-3-
yl)-6-chloro-8-fluoro-2-((1-
(piperidin-1-yl)propan-2-
yl)oxy)quinazolin-7-yl)-7-
fluorobenzo[d]thiazol-2-amine

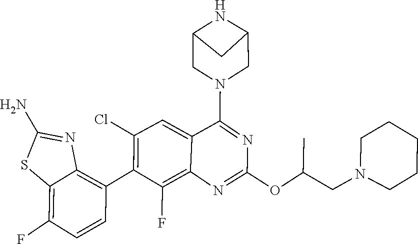 [SMILES: CC(CN1CCCCC1)Oc1nc(N2CC3CC(C2)N3)c2cc(Cl)c(-c3ccc(F)c4sc(N)nc34)c(F)c2n1]

E6: 4-(4-(3,6-
diazabicyclo[3.1.1]heptan-3-yl)-
6-chloro-8-fluoro-2-((1-
(piperidin-1-yl)propan-2-
yl)oxy)quinazolin-7-yl)-7-
fluorobenzo[d]thiazol-2-amine

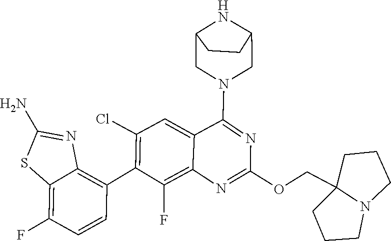 [SMILES: Nc1nc2c(-c3c(Cl)cc4c(N5CC6CCC(C5)N6)nc(OCC56CCCN5CCC6)nc4c3F)ccc(F)c2s1]

E7: 4-(4-(3,8-
diazabicyclo[3.2.1]octan-3-yl)-6-
chloro-8-fluoro-2-((tetrahydro-
1H-pyrrolizin-7a(5H)-
yl)methoxy)quinazolin-7-yl)-7-
fluorobenzo[d]thiazol-2-amine

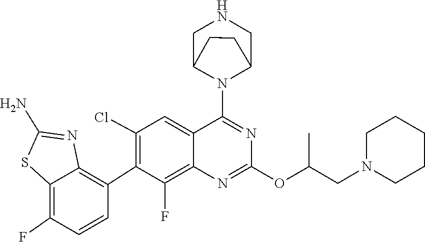 [SMILES: CC(CN1CCCCC1)Oc1nc(N2C3CCC2CNC3)c2cc(Cl)c(-c3ccc(F)c4sc(N)nc34)c(F)c2n1]

E8: 4-(4-(3,8-
diazabicyclo[3.2.1]octan-8-
yl)-6-chloro-8-fluoro-2-((1-
(piperidin-1-yl)propan-2-
yl)oxy)quinazolin-7-yl)-7-
fluorobenzo[d]thiazol-2-amine

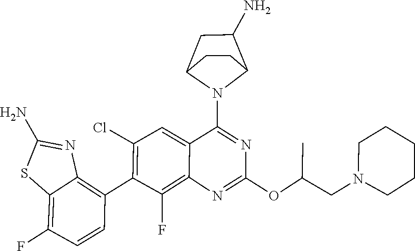 [SMILES: CC(CN1CCCCC1)Oc1nc(N2C3CCC2C(N)C3)c2cc(Cl)c(-c3ccc(F)c4sc(N)nc34)c(F)c2n1]

E9: 4-(4-(2-amino-7-
azabicyclo[2.2.1]heptan-7-yl)-6-
chloro-8-fluoro-2-((1-(piperidin-
1-yl)propan-2-yl)oxy)quinazolin-
7-yl)-7-fluorobenzo[d]thiazol-2-
amine

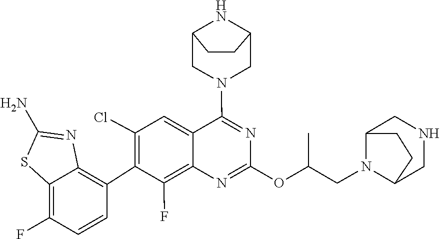 [SMILES: CC(CN1C2CCC1CNC2)Oc1nc(N2CC3CCC(C2)N3)c2cc(Cl)c(-c3ccc(F)c4sc(N)nc34)c(F)c2n1]

E10: 4-(2-((1-(3,8-
diazabicyclo[3.2.1]octan-8-
yl)propan-2-yl)oxy)-4-(3,8-
diazabicyclo[3.2.1]octan-3-yl)-6-
chloro-8-fluoroquinazolin-7-yl)-
7-fluorobenzo[d]thiazol-2-amine

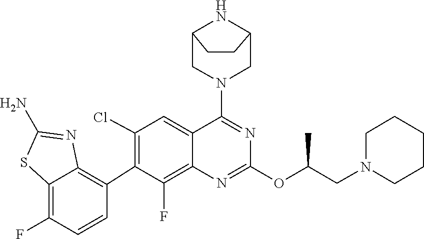 [SMILES: C[C@@H](CN1CCCCC1)Oc1nc(N2CC3CCC(C2)N3)c2cc(Cl)c(-c3ccc(F)c4sc(N)nc34)c(F)c2n1]

E11: 4-(4-(3,8-
diazabicyclo[3.2.1]octan-3-
yl)-6-chloro-8-fluoro-2-(((S)-
1-(piperidin-1-yl)propan-2-
yl)oxy)quinazolin-7-yl)-7-
fluorobenzo[d]thiazol-2-amine

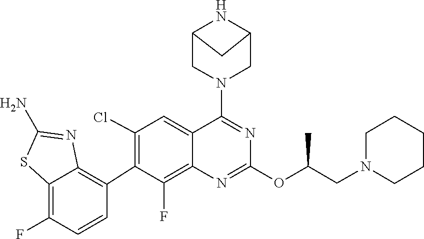 [SMILES: C[C@@H](CN1CCCCC1)Oc1nc(N2CC3CC(C2)N3)c2cc(Cl)c(-c3ccc(F)c4sc(N)nc34)c(F)c2n1]

E12: 4-(4-(3,6-
diazabicyclo[3.1.1]heptan-3-yl)-
6-chloro-8-fluoro-2-(((S)-1-
(piperidin-1-yl)propan-2-
yl)oxy)quinazolin-7-yl)-7-
fluorobenzo[d]thiazol-2-amine

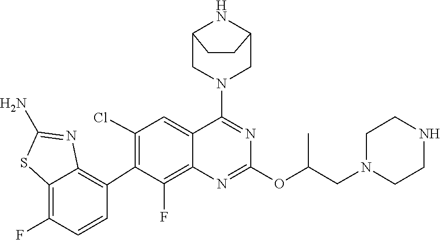 [SMILES: CC(CN1CCNCC1)Oc1nc(N2CC3CCC(C2)N3)c2cc(Cl)c(-c3ccc(F)c4sc(N)nc34)c(F)c2n1]

E13: 4-(4-(3,8-
diazabicyclo[3.2.1]octan-3-yl)-6-
chloro-8-fluoro-2-((1-(piperazin-
1-yl)propan-2-yl)oxy)quinazolin-
7-yl)-7-fluorobenzo[d]thiazol-2-
amine

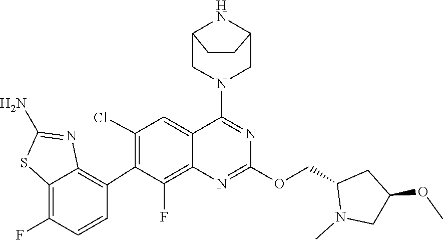 [SMILES: CO[C@@H]1C[C@@H](COc2nc(N3CC4CCC(C3)N4)c3cc(Cl)c(-c4ccc(F)c5sc(N)nc45)c(F)c3n2)N(C)C1]

E14: 4-(4-(3,8-
diazabicyclo[3.2.1]octan-3-
yl)-6-chloro-8-fluoro-2-
(((2S,4R)-4-methoxy-1-
methylpyrrolidin-2-
yl)methoxy)quinazolin-7-yl)-
7-fluorobenzo[d]thiazol-2-
amine

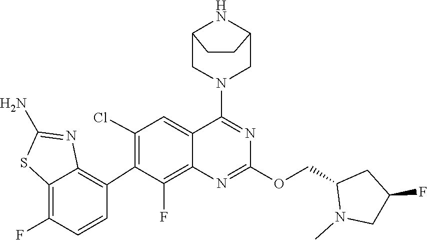 [SMILES: CN1C[C@H](F)C[C@H]1COc1nc(N2CC3CCC(C2)N3)c2cc(Cl)c(-c3ccc(F)c4sc(N)nc34)c(F)c2n1]

E15: 4-(4-(3,8-
diazabicyclo[3.2.1]octan-3-yl)-6-
chloro-8-fluoro-2-(((2S,4R)-4-
fluoro-1-methylpyrrolidin-2-
yl)methoxy)quinazolin-7-yl)-7-
fluorobenzo[d]thiazol-2-amine

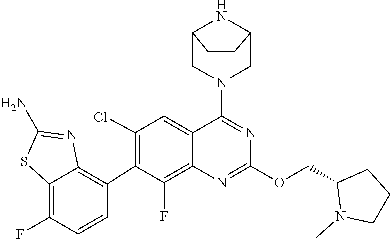 [SMILES: CN1CCC[C@H]1COc1nc(N2CC3CCC(C2)N3)c2cc(Cl)c(-c3ccc(F)c4sc(N)nc34)c(F)c2n1]

E16: 4-(4-(3,8-
diazabicyclo[3.2.1]octan-3-yl)-6-
chloro-8-fluoro-2-(((S)-1-
methylpyrrolidin-2-
yl)methoxy)quinazolin-7-yl)-7-
fluorobenzo[d]thiazol-2-amine

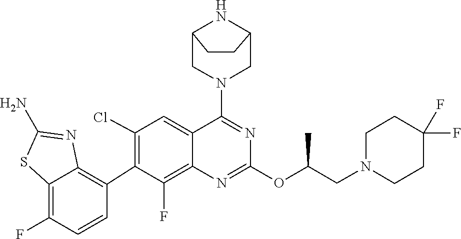 [SMILES: C[C@@H](CN1CCC(F)(F)CC1)Oc1nc(N2CC3CCC(C2)N3)c2cc(Cl)c(-c3ccc(F)c4sc(N)nc34)c(F)c2n1]

E17: 4-(4-(3,8-
diazabicyclo[3.2.1]octan-3-
yl)-6-chloro-2-(((S)-1-(4,4-
difluoropiperidin-1-yl)propan-
2-yl)oxy)-8-fluoroquinazolin-
7-yl)-7-fluorobenzo[d]thiazol-
2-amine

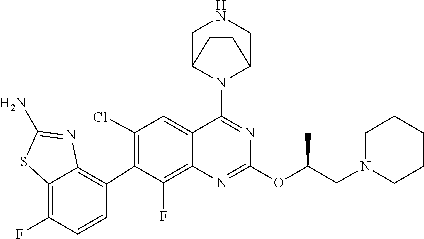 [SMILES: C[C@@H](CN1CCCCC1)Oc1nc(N2C3CCC2CNC3)c2cc(Cl)c(-c3ccc(F)c4sc(N)nc34)c(F)c2n1]

E18: 4-(4-(3,8-
diazabicyclo[3.2.1]octan-8-yl)-6-
chloro-8-fluoro-2-(((S)-1-
(piperidin-1-yl)propan-2-
yl)oxy)quinazolin-7-yl)-7-
fluorobenzo[d]thiazol-2-amine

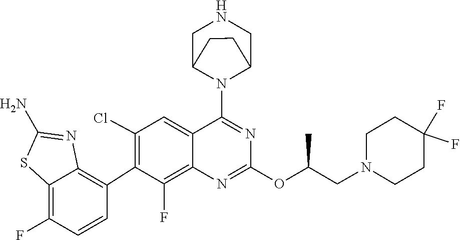 [SMILES: C[C@@H](CN1CCC(F)(F)CC1)Oc1nc(N2C3CCC2CNC3)c2cc(Cl)c(-c3ccc(F)c4sc(N)nc34)c(F)c2n1]

E19: 4-(4-(3,8-
diazabicyclo[3.2.1]octan-8-yl)-6-
chloro-2-(((S)-1-(4,4-
difluoropiperidin-1-yl)propan-2-
yl)oxy)-8-fluoroquinazolin-7-yl)-
7-fluorobenzo[d]thiazol-2-amine

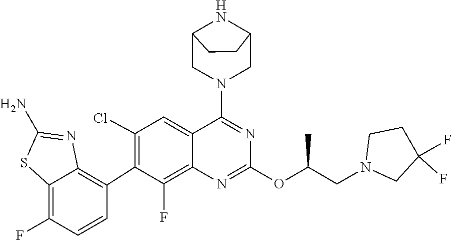 [SMILES: C[C@@H](CN1CCC(F)(F)C1)Oc1nc(N2CC3CCC(C2)N3)c2cc(Cl)c(-c3ccc(F)c4sc(N)nc34)c(F)c2n1]

E20: 4-(4-(3,8-
diazabicyclo[3.2.1]octan-3-
yl)-6-chloro-2-(((S)-1-(3,3-
difluoropyrrolidin-1-
yl)propan-2-yl)oxy)-8-
fluoroquinazolin-7-yl)-7-
fluorobenzo[d]thiazol-2-amine

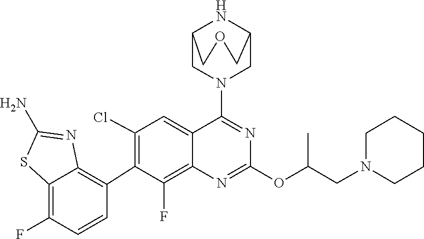 [SMILES: CC(CN1CCCCC1)Oc1nc(N2CC3COCC(C2)N3)c2cc(Cl)c(-c3ccc(F)c4sc(N)nc34)c(F)c2n1]

E21: 4-(4-(3-oxa-7,9-
diazabicyclo[3.3.1]nonan-7-yl)-6-
chloro-8-fluoro-2-((1-(piperidin-
1-yl)propan-2-yl)oxy)quinazolin-
7-yl)-7-fluorobenzo[d]thiazol-2-
amine

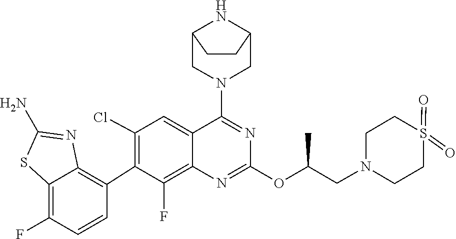 [SMILES: C[C@@H](CN1CCS(=O)(=O)CC1)Oc1nc(N2CC3CCC(C2)N3)c2cc(Cl)c(-c3ccc(F)c4sc(N)nc34)c(F)c2n1]

E22: 4-((2S)-2-((7-(2-amino-7-
fluorobenzo[d]thiazol-4-yl)-4-
(3,8-diazabicyclo[3.2.1]octan-3-
yl)-6-chloro-8-fluoroquinazolin-
2-yl)oxy)propyl)thiomorpholine
1,1-dioxide

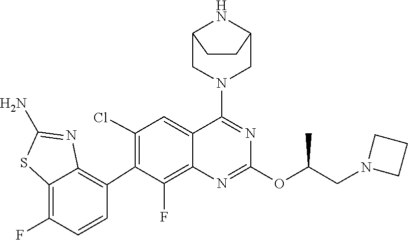 [SMILES: C[C@@H](CN1CCC1)Oc1nc(N2CC3CCC(C2)N3)c2cc(Cl)c(-c3ccc(F)c4sc(N)nc34)c(F)c2n1]

E23: 4-(2-(((S)-1-(azetidin-1-
yl)propan-2-y1)oxy)-4-(3,8-
diazabicyclo[3.2.1]octan-3-
yl)-6-chloro-8-
fluoroquinazolin-7-yl)-7-
fluorobenzo[d]thiazol-2-amine

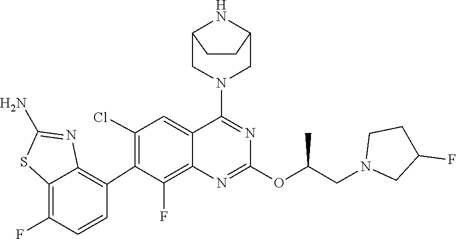 [SMILES: C[C@@H](CN1CCC(F)C1)Oc1nc(N2CC3CCC(C2)N3)c2cc(Cl)c(-c3ccc(F)c4sc(N)nc34)c(F)c2n1]

E24: 4-(4-(3,8-
diazabicyclo[3.2.1]octan-3-yl)-6-
chloro-8-fluoro-2-(((2S)-1-(3-
fluoropyrrolidin-1-yl)propan-2-
yl)oxy)quinazolin-7-yl)-7-
fluorobenzo[d]thiazol-2-amine

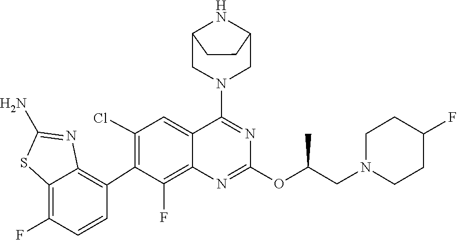 [SMILES: C[C@@H](CN1CCC(F)CC1)Oc1nc(N2CC3CCC(C2)N3)c2cc(Cl)c(-c3ccc(F)c4sc(N)nc34)c(F)c2n1]

E25: 4-(4-(3,8-
diazabicyclo[3.2.1]octan-3-yl)-6-
chloro-8-fluoro-2-(((S)-1-(4-
fluoropiperidin-1-yl)propan-2-
yl)oxy)quinazolin-7-yl)-7-
fluorobenzo[d]thiazol-2-amine

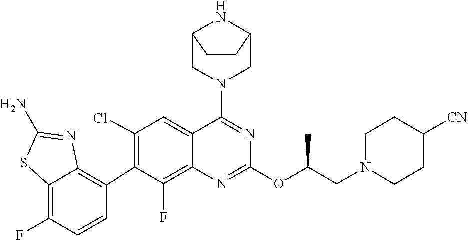 [SMILES: C[C@@H](CN1CCC(C#N)CC1)Oc1nc(N2CC3CCC(C2)N3)c2cc(Cl)c(-c3ccc(F)c4sc(N)nc34)c(F)c2n1]

E26: 1-((2S)-2-((7-(2-amino-
7-fluorobenzo[d]thiazol-4-yl)-
4-(3,8-
diazabicyclo[3.2.1]octan-3-
yl)-6-chloro-8-
fluoroquinazolin-2-
yl)oxy)propyl)piperidine-4-
carbonitrile

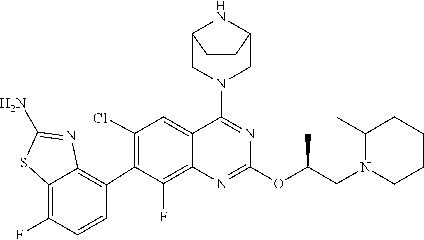 [SMILES: CC1CCCCN1C[C@H](C)Oc1nc(N2CC3CCC(C2)N3)c2cc(Cl)c(-c3ccc(F)c4sc(N)nc34)c(F)c2n1]

E27: 4-(4-(3,8-
diazabicyclo[3.2.1]octan-3-yl)-6-
chloro-8-fluoro-2-(((2S)-1-(2-
methylpiperidin-1-yl)propan-2-
yl)oxy)quinazolin-7-yl)-7-
fluorobenzo[d]thiazol-2-amine

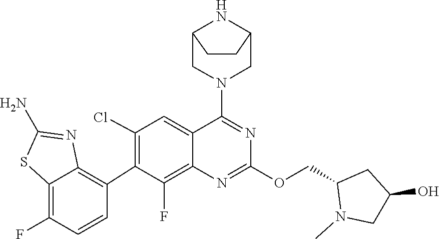 [SMILES: CN1C[C@H](O)C[C@H]1COc1nc(N2CC3CCC(C2)N3)c2cc(Cl)c(-c3ccc(F)c4sc(N)nc34)c(F)c2n1]

E28: (3R,5S)-5-(((7-(2-amino-7-
fluorobenzo[d]thiazol-4-yl)-4-
(3,8-diazabicyclo[3.2.1]octan-3-
yl)-6-chloro-8-fluoroquinazolin-
2-yl)oxy)methyl)-1-
methylpyrrolidin-3-ol

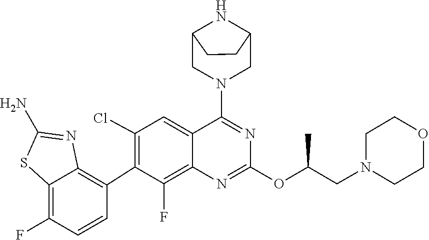 [SMILES: C[C@@H](CN1CCOCC1)Oc1nc(N2CC3CCC(C2)N3)c2cc(Cl)c(-c3ccc(F)c4sc(N)nc34)c(F)c2n1]

E29: 4-(4-(3,8-
diazabicyclo[3.2.1]octan-3-
yl)-6-chloro-8-fluoro-2-(((S)-
1-morpholinopropan-2-
yl)oxy)quinazolin-7-yl)-7-
fluorobenzo[d]thiazol-2-amine

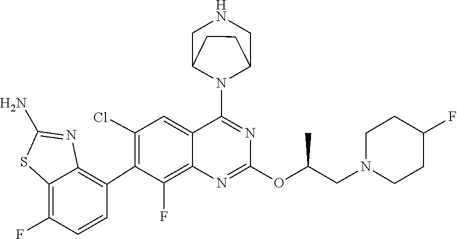 [SMILES: C[C@@H](CN1CCC(F)CC1)Oc1nc(N2C3CCC2CNC3)c2cc(Cl)c(-c3ccc(F)c4sc(N)nc34)c(F)c2n1]

E30: 4-(4-(3,8-
diazabicyclo[3.2.1]octan-8-yl)-6-
chloro-8-fluoro-2-(((S)-1-(4-
fluoropiperidin-1-yl)propan-2-
yl)oxy)quinazolin-7-yl)-7-
fluorobenzo[d]thiazol-2-amine

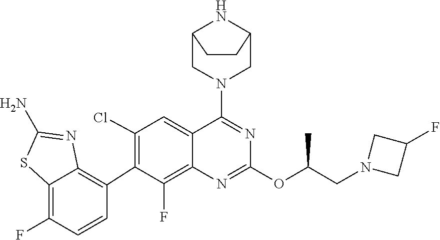 [SMILES: C[C@@H](CN1CC(F)C1)Oc1nc(N2CC3CCC(C2)N3)c2cc(Cl)c(-c3ccc(F)c4sc(N)nc34)c(F)c2n1]

E31: 4-(4-(3,8-
diazabicyclo[3.2.1]octan-3-yl)-6-
chloro-8-fluoro-2-(((S)-1-(3-
fluoroazetidin-1-yl)propan-2-
yl)oxy)quinazolin-7-yl)-7-
fluorobenzo[d]thiazol-2-amine

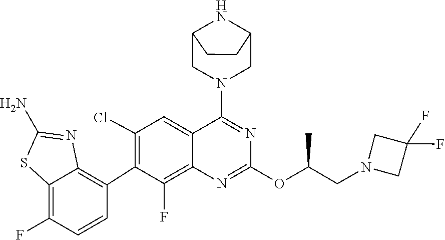 [SMILES: C[C@@H](CN1CC(F)(F)C1)Oc1nc(N2CC3CCC(C2)N3)c2cc(Cl)c(-c3ccc(F)c4sc(N)nc34)c(F)c2n1]

E32: 4-(4-(3,8-
diazabicyclo[3.2.1]octan-3-
yl)-6-chloro-2-(((S)-1-(3,3-
difluoroazetidin-1-yl)propan-
2-yl)oxy)-8-fluoroquinazolin-
7-yl)-7-fluorobenzo[d]thiazol-
2-amine

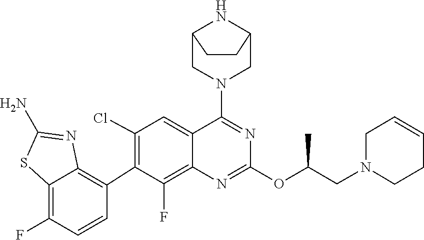 [SMILES: C[C@@H](CN1CC=CCC1)Oc1nc(N2CC3CCC(C2)N3)c2cc(Cl)c(-c3ccc(F)c4sc(N)nc34)c(F)c2n1]

E33: 4-(4-(3,8-
diazabicyclo[3.2.1]octan-3-yl)-6-
chloro-2-(((S)-1-(3,6-
dihydropyridin-1(2H)-yl)propan-
2-yl)oxy)-8-fluoroquinazolin-7-
yl)-7-fluorobenzo[d]thiazol-2-
amine

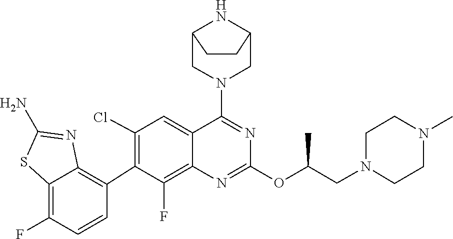 [SMILES: C[C@@H](CN1CCN(C)CC1)Oc1nc(N2CC3CCC(C2)N3)c2cc(Cl)c(-c3ccc(F)c4sc(N)nc34)c(F)c2n1]

E34: 4-(4-(3,8-
diazabicyclo[3.2.1]octan-3-yl)-6-
chloro-8-fluoro-2-(((S)-1-(4-
methylpiperazin-1-yl)propan-2-
yl)oxy)quinazolin-7-yl)-7-
fluorobenzo[d]thiazol-2-amine

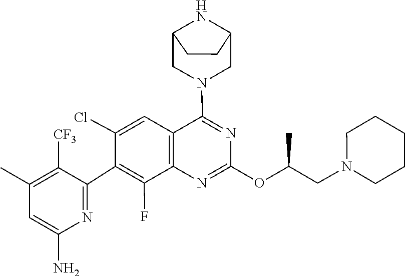 [SMILES: Cc1cc(N)nc(-c2c(Cl)cc3c(N4CC5CCC(C4)N5)nc(O[C@@H](C)CN4CCCCC4)nc3c2F)c1C(F)(F)F]

E35: 6-(4-(3,8-
diazabicyclo[3.2.1]octan-3-
yl)-6-chloro-8-fluoro-2-(((S)-
1-(piperidin-1-yl)propan-2-
yl)oxy)quinazolin-7-yl)-4-
methyl-5-
(trifluoromethyl)pyridin-2-
amine

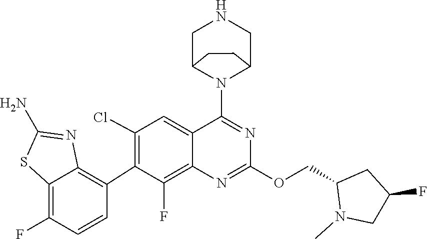 [SMILES: CN1C[C@H](F)C[C@H]1COc1nc(N2C3CCC2CNC3)c2cc(Cl)c(-c3ccc(F)c4sc(N)nc34)c(F)c2n1]

E36: 4-(4-(3,8-
diazabicyclo[3.2.1]octan-8-yl)-6-
chloro-8-fluoro-2-(((2S,4R)-4-
fluoro-1-methylpyrrolidin-2-
yl)methoxy)quinazolin-7-yl)-7-
fluorobenzo[d]thiazol-2-amine

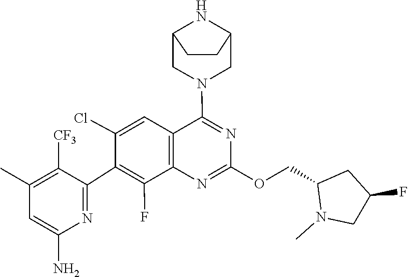 [SMILES: Cc1cc(N)nc(-c2c(Cl)cc3c(N4CC5CCC(C4)N5)nc(OC[C@@H]4C[C@@H](F)CN4C)nc3c2F)c1C(F)(F)F]

E37: 6-(4-(3,8-
diazabicyclo[3.2.1]octan-3-yl)-6-
chloro-8-fluoro-2-(((2S,4R)-4-
fluoro-1-methylpyrrolidin-2-
yl)methoxy)quinazolin-7-yl)-4-
methyl-5-
(trifluoromethyl)pyridin-2-amine

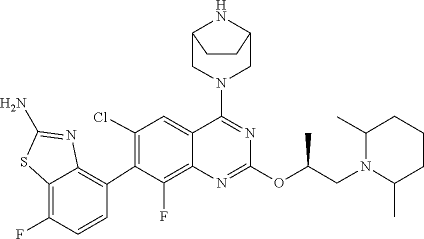 [SMILES: CC1CCCC(C)N1C[C@H](C)Oc1nc(N2CC3CCC(C2)N3)c2cc(Cl)c(-c3ccc(F)c4sc(N)nc34)c(F)c2n1]

E38: 4-(4-(3,8-
diazabicyclo[3.2.1]octan-3-
yl)-6-chloro-2-(((2S)-1-(2,6-
dimethylpiperidin-1-
yl)propan-2-yl)oxy)-8-
fluoroquinazolin-7-yl)-7-
fluorobenzo[d]thiazol-2-amine

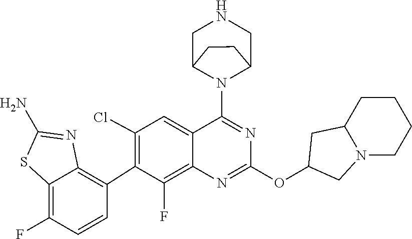 [SMILES: Nc1nc2c(-c3c(Cl)cc4c(N5C6CCC5CNC6)nc(OC5CC6CCCCN6C5)nc4c3F)ccc(F)c2s1]

E39: 4-(4-(3,8-
diazabicyclo[3.2.1]octan-8-yl)-6-
chloro-8-fluoro-2-
((octahydroindolizin-2-
yl)oxy)quinazolin-7-yl)-7-
fluorobenzo[d]thiazol-2-amine

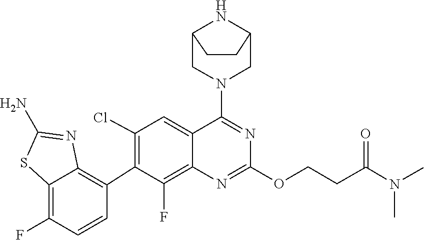 [SMILES: CN(C)C(=O)CCOc1nc(N2CC3CCC(C2)N3)c2cc(Cl)c(-c3ccc(F)c4sc(N)nc34)c(F)c2n1]

E40: 3-((7-(2-amino-7-
fluorobenzo[d]thiazol-4-yl)-4-
(3,8-diazabicyclo[3.2.1]octan-3-
yl)-6-chloro-8-fluoroquinazolin-
2-yl)oxy)-N,N-
dimethylpropanamide

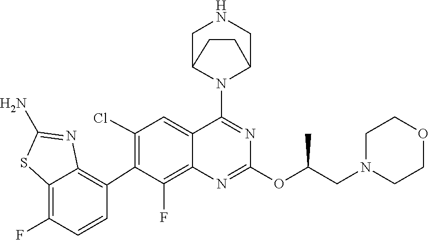 [SMILES: C[C@@H](CN1CCOCC1)Oc1nc(N2C3CCC2CNC3)c2cc(Cl)c(-c3ccc(F)c4sc(N)nc34)c(F)c2n1]

E41: 4-(4-(3,8-
diazabicyclo[3.2.1]octan-8-
yl)-6-chloro-8-fluoro-2-(((S)-
1-morpholinopropan-2-
yl)oxy)quinazolin-7-yl)-7-
fluorobenzo[d]thiazol-2-amine

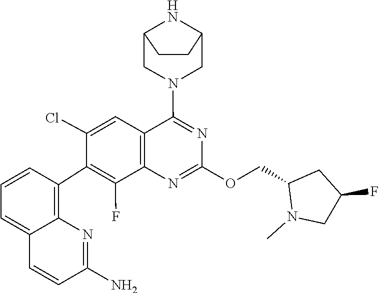 [SMILES: CN1C[C@H](F)C[C@H]1COc1nc(N2CC3CCC(C2)N3)c2cc(Cl)c(-c3cccc4ccc(N)nc34)c(F)c2n1]

E42: 8-(4-(3,8-
diazabicyclo[3.2.1]octan-3-yl)-6-
chloro-8-fluoro-2-(((2S,4R)-4-
fluoro-1-methylpyrrolidin-2-
yl)methoxy)quinazolin-7-
yl)quinolin-2-amine

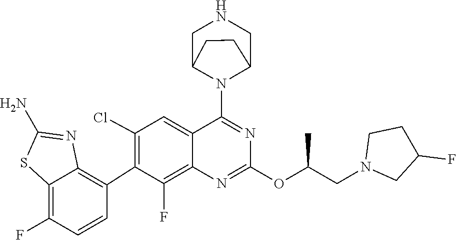 [SMILES: C[C@@H](CN1CCC(F)C1)Oc1nc(N2C3CCC2CNC3)c2cc(Cl)c(-c3ccc(F)c4sc(N)nc34)c(F)c2n1]

E43: 4-(4-(3,8-
diazabicyclo[3.2.1]octan-8-yl)-6-
chloro-8-fluoro-2-(((2S)-1-(3-
fluoropyrrolidin-1-yl)propan-2-
yl)oxy)quinazolin-7-yl)-7-
fluorobenzo[d]thiazol-2-amine

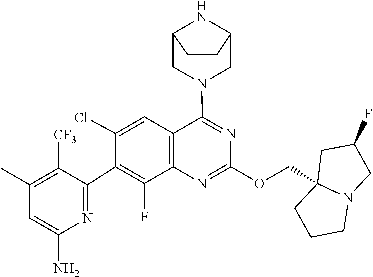 [SMILES: Cc1cc(N)nc(-c2c(Cl)cc3c(N4CC5CCC(C4)N5)nc(OC[C@@]45CCCN4C[C@H](F)C5)nc3c2F)c1C(F)(F)F]

E49: 6-(4-(3,8-
diazabicyclo[3.2.1]octan-3-
yl)-6-chloro-8-fluoro-2-
(((2R,7aS)-2-fluorotetrahydro-
1H-pyrrolizin-7a(5H)-
yl)methoxy)quinazolin-7-yl)-
4-methyl-5-
(trifluoromethyl)pyridin-2-
amine

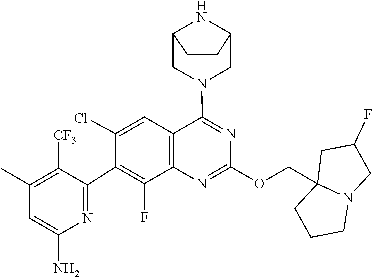 [SMILES: Cc1cc(N)nc(-c2c(Cl)cc3c(N4CC5CCC(C4)N5)nc(OCC45CCCN4CC(F)C5)nc3c2F)c1C(F)(F)F]

E50: 6-(4-(3,8-
diazabicyclo[3.2.1]octan-3-yl)-6-
chloro-8-fluoro-2-((2-
fluorotetrahydro-1H-pyrrolizin-
7a(5H)-yl)methoxy)quinazolin-7-
yl)-4-methyl-5-
(trifluoromethyl)pyridin-2-amine

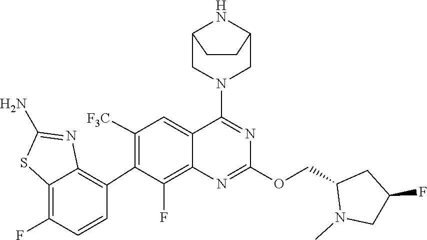 [SMILES: CN1C[C@H](F)C[C@H]1COc1nc(N2CC3CCC(C2)N3)c2cc(C(F)(F)F)c(-c3ccc(F)c4sc(N)nc34)c(F)c2n1]

E51: 4-(4-(3,8-
diazabicyclo[3.2.1]octan-3-yl)-8-
fluoro-2-(((2S,4R)-4-fluoro-1-
methylpyrrolidin-2-yl)methoxy)-
6-(trifluoromethyl)quinazolin-7-
yl)-7-fluorobenzo[d]thiazol-2-
amine

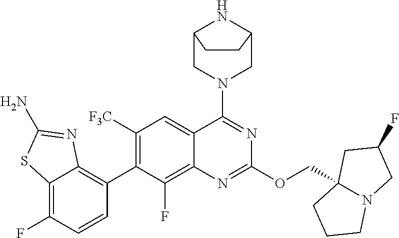 [SMILES: Nc1nc2c(-c3c(C(F)(F)F)cc4c(N5CC6CCC(C5)N6)nc(OC[C@@]56CCCN5C[C@H](F)C6)nc4c3F)ccc(F)c2s1]

E52: 4-(4-(3,8-
diazabicyclo[3.2.1]octan-3-
yl)-8-fluoro-2-(((2R,7aS)-2-
fluorotetrahydro-1H-
pyrrolizin-7a(5H)-
yl)methoxy)-6-
(trifluoromethyl)quinazolin-7-
yl)-7-fluorobenzo[d]thiazol-2-
amine

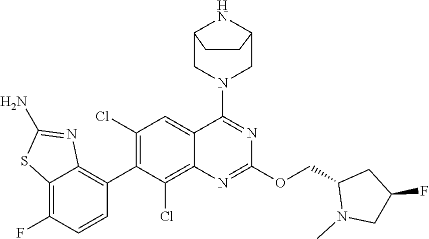 [SMILES: CN1C[C@H](F)C[C@H]1COc1nc(N2CC3CCC(C2)N3)c2cc(Cl)c(-c3ccc(F)c4sc(N)nc34)c(Cl)c2n1]

E53: 4-(4-(3,8-
diazabicyclo[3.2.1]octan-3-yl)-
6,8-dichloro-2-(((2S,4R)-4-
fluoro-1-methylpyrrolidin-2-
yl)methoxy)quinazolin-7-yl)-7-
fluorobenzo[d]thiazol-2-amine

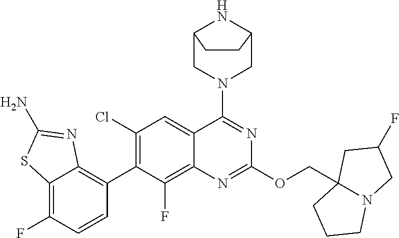 [SMILES: Nc1nc2c(-c3c(Cl)cc4c(N5CC6CCC(C5)N6)nc(OCC56CCCN5CC(F)C6)nc4c3F)ccc(F)c2s1]

E54: 4-(4-(3,8-
diazabicyclo[3.2.1]octan-3-yl)-6-
chloro-8-fluoro-2-((2-
fluorotetrahydro-1H-pyrrolizin-
7a(5H)-yl)methoxy)quinazolin-7-
yl)-7-fluorobenzo[d]thiazol-2-
amine

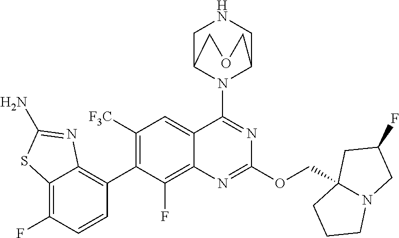 [SMILES: Nc1nc2c(-c3c(C(F)(F)F)cc4c(N5C6CNCC5COC6)nc(OC[C@@]56CCCN5C[C@H](F)C6)nc4c3F)ccc(F)c2s1]

E55: 4-(4-(3-oxa-7,9-
diazabicyclo[3.3.1]nonan-9-
yl)-8-fluoro-2-(((2R,7aS)-2-
fluorotetrahydro-1H-
pyrrolizin-7a(5H)-
yl)methoxy)-6-
(trifluoromethyl)quinazolin-7-
yl)-7-fluorobenzo[d]thiazol-2-
amine

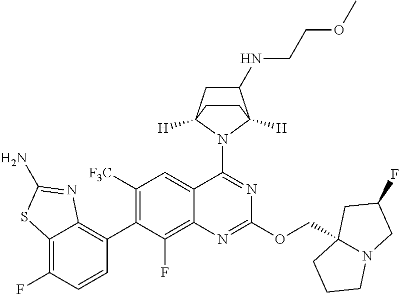 [SMILES: COCCNC1C[C@H]2CC[C@@H]1N2c1nc(OC[C@@]23CCCN2C[C@H](F)C3)nc2c(F)c(-c3ccc(F)c4sc(N)nc34)c(C(F)(F)F)cc12]

E58: 7-fluoro-4-(8-fluoro-2-
(((2R,7aS)-2-fluorotetrahydro-
1H-pyrrolizin-7a(5H)-
yl)methoxy)-4-((1S,4R)-2-((2-
methoxyethyl)amino)-7-
azabicyclo[2.2.1]heptan-7-yl)-6-
(trifluoromethyl)quinazolin-7-
yl)benzo[d]thiazol-2-amine

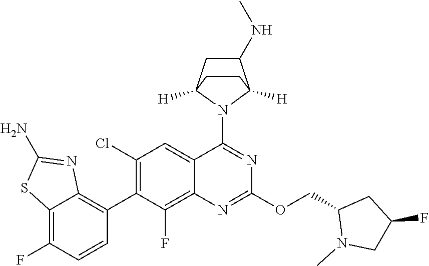 [SMILES: CNC1C[C@H]2CC[C@@H]1N2c1nc(OC[C@@H]2C[C@@H](F)CN2C)nc2c(F)c(-c3ccc(F)c4sc(N)nc34)c(Cl)cc12]

E59: 4-(6-chloro-8-fluoro-2-
(((2S,4R)-4-fluoro-1-
methylpyrrolidin-2-yl)methoxy)-
4-((1S,4R)-2-(methylamino)-7-
azabicyclo[2.2.1]heptan-7-
yl)quinazolin-7-yl)-7-
fluorobenzo[d]thiazol-2-amine

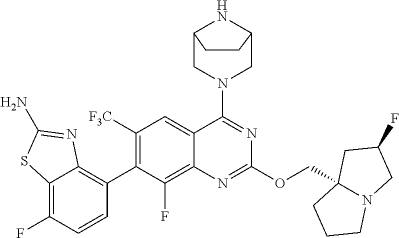 [SMILES: Nc1nc2c(-c3c(C(F)(F)F)cc4c(N5CC6CCC(C5)N6)nc(OC[C@@]56CCCN5C[C@H](F)C6)nc4c3F)ccc(F)c2s1]

E60: 4-(4-(3,8-
diazabicyclo[3.2.1]octan-8-
yl)-8-fluoro-2-(((2R,7aS)-2-
fluorotetrahydro-1H-
pyrrolizin-7a(5H)-
yl)methoxy)-6-
(trifluoromethyl)quinazolin-7-
yl)-7-fluorobenzo[d]thiazol-2-
amine

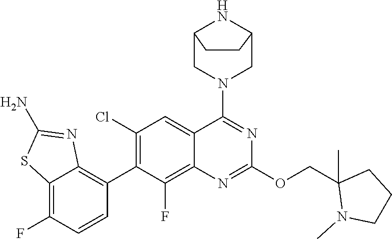 [SMILES: CN1CCCC1(C)COc1nc(N2CC3CCC(C2)N3)c2cc(Cl)c(-c3ccc(F)c4sc(N)nc34)c(F)c2n1]

E61: 4-(4-(3,8-
diazabicyclo[3.2.1]octan-3-yl)-6-
chloro-2-((1,2-
dimethylpyrrolidin-2-
yl)methoxy)-8-fluoroquinazolin-
7-yl)-7-fluorobenzo[d]thiazol-2-
amine

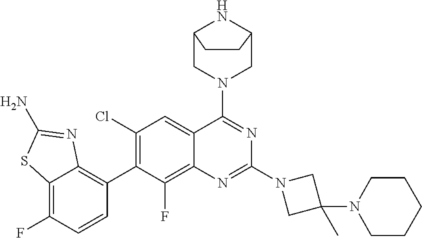 [SMILES: CC1(N2CCCCC2)CN(c2nc(N3CC4CCC(C3)N4)c3cc(Cl)c(-c4ccc(F)c5sc(N)nc45)c(F)c3n2)C1]

E62: 4-(4-(3,8-
diazabicyclo[3.2.1]octan-3-yl)-6-
chloro-8-fluoro-2-(3-methyl-3-
(piperidin-1-yl)azetidin-1-
yl)quinazolin-7-yl)-7-
fluorobenzo[d]thiazol-2-amine

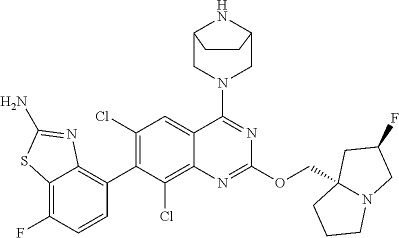 [SMILES: Nc1nc2c(-c3c(Cl)cc4c(N5CC6CCC(C5)N6)nc(OC[C@@]56CCCN5C[C@H](F)C6)nc4c3Cl)ccc(F)c2s1]

E63: 4-(4-(3,8-
diazabicyclo[3.2.1]octan-3-
yl)-6,8-dichloro-2-(((2R,7aS)-
2-fluorotetrahydro-1H-
pyrrolizin-7a(5H)-
yl)methoxy)quinazolin-7-yl)-
7-fluorobenzo[d]thiazol-2-
amine

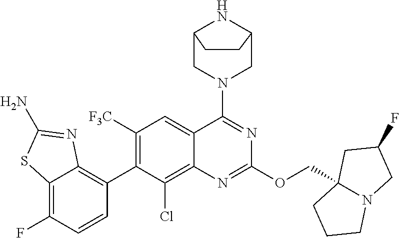 [SMILES: Nc1nc2c(-c3c(C(F)(F)F)cc4c(N5CC6CCC(C5)N6)nc(OC[C@@]56CCCN5C[C@H](F)C6)nc4c3Cl)ccc(F)c2s1]

E64: 4-(4-(3,8-
diazabicyclo[3.2.1]octan-3-yl)-8-
chloro-2-(((2R,7aS)-2-
fluorotetrahydro-1H-pyrrolizin-
7a(5H)-yl)methoxy)-6-
(trifluoromethyl)quinazolin-7-yl)-
7-fluorobenzo[d]thiazol-2-amine

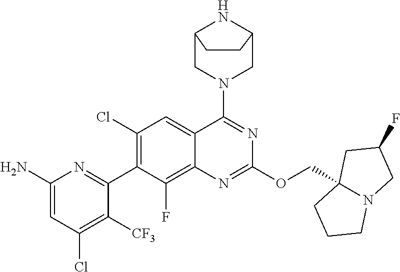 [SMILES: Nc1cc(Cl)c(C(F)(F)F)c(-c2c(Cl)cc3c(N4CC5CCC(C4)N5)nc(OC[C@@]45CCCN4C[C@H](F)C5)nc3c2F)n1]

E65: 6-(4-(3,8-
diazabicyclo[3.2.1]octan-3-yl)-6-
chloro-8-fluoro-2-(((2R,7aS)-2-
fluorotetrahydro-1H-pyrrolizin-
7a(5H)-yl)methoxy)quinazolin-7-
yl)-4-chloro-5-
(trifluoromethyl)pyridin-2-amine

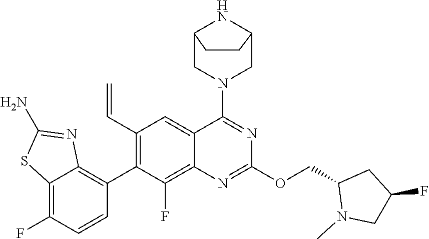 [SMILES: C=Cc1cc2c(N3CC4CCC(C3)N4)nc(OC[C@@H]3C[C@@H](F)CN3C)nc2c(F)c1-c1ccc(F)c2sc(N)nc12]

E66: 4-(4-(3,8-
diazabicyclo[3.2.1]octan-3-
yl)-8-fluoro-2-(((2S,4R)-4-
fluoro-1-methylpyrrolidin-2-
yl)methoxy)-6-
vinylquinazolin-7-yl)-7-
fluorobenzo[d]thiazol-2-amine

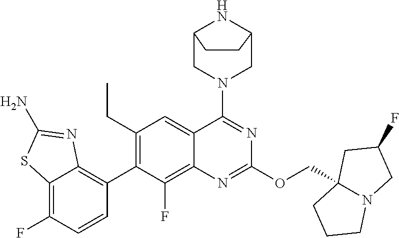 [SMILES: CCc1cc2c(N3CC4CCC(C3)N4)nc(OC[C@@]34CCCN3C[C@H](F)C4)nc2c(F)c1-c1ccc(F)c2sc(N)nc12]

E67: 4-(4-(3,8-
diazabicyclo[3.2.1]octan-3-yl)-6-
ethyl-8-fluoro-2-(((2R,7aS)-2-
fluorotetrahydro-1H-pyrrolizin-
7a(5H)-yl)methoxy)quinazolin-7-
yl)-7-fluorobenzo[d]thiazol-2-
amine

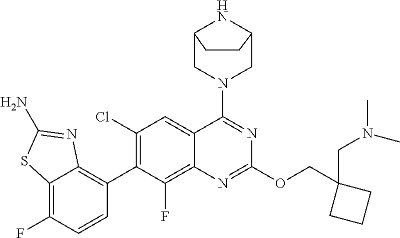 [SMILES: CN(C)CC1(COc2nc(N3CC4CCC(C3)N4)c3cc(Cl)c(-c4ccc(F)c5sc(N)nc45)c(F)c3n2)CCC1]

E68: 4-(4-(3,8-
diazabicyclo[3.2.1]octan-3-yl)-6-
chloro-2-((1-
((dimethylamino)methyl)
cyclobutyl)methoxy)-8-
fluoroquinazolin-7-yl)-7-
fluorobenzo[d]thiazol-2-amine

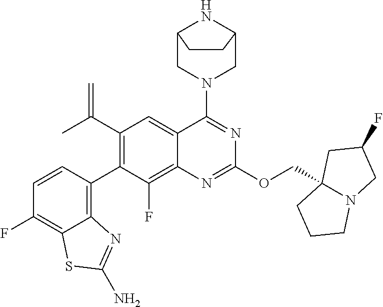 [SMILES: C=C(C)c1cc2c(N3CC4CCC(C3)N4)nc(OC[C@@]34CCCN3C[C@H](F)C4)nc2c(F)c1-c1ccc(F)c2sc(N)nc12]

E69: 4-(4-(3,8-
diazabicyclo[3.2.1]octan-3-
yl)-8-fluoro-2-(((2R,7aS)-2-
fluorotetrahydro-1H-
pyrrolizin-7a(5H)-
yl)methoxy)-6-(prop-1-en-2-
yl)quinazolin-7-yl)-7-
fluorobenzo[d]thiazol-2-amine

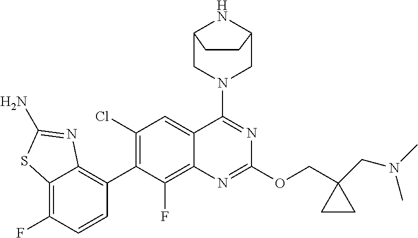 [SMILES: CN(C)CC1(COc2nc(N3CC4CCC(C3)N4)c3cc(Cl)c(-c4ccc(F)c5sc(N)nc45)c(F)c3n2)CC1]

E70: 4-(4-(3,8-
diazabicyclo[3.2.1]octan-3-yl)-6-
chloro-2-((1-
((dimethylamino)methyl)
cyclopropyl)methoxy)-8-
fluoroquinazolin-7-yl)-7-
fluorobenzo[d]thiazol-2-amine

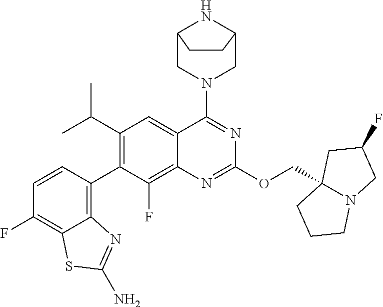 [SMILES: CC(C)c1cc2c(N3CC4CCC(C3)N4)nc(OC[C@@]34CCCN3C[C@H](F)C4)nc2c(F)c1-c1ccc(F)c2sc(N)nc12]

E71: 4-(4-(3,8-
diazabicyclo[3.2.1]octan-3-yl)-8-
fluoro-2-(((2R,7aS)-2-
fluorotetrahydro-1H-pyrrolizin-
7a(5H)-yl)methoxy)-6-
isopropylquinazolin-7-yl)-7-
fluorobenzo[d]thiazol-2-amine

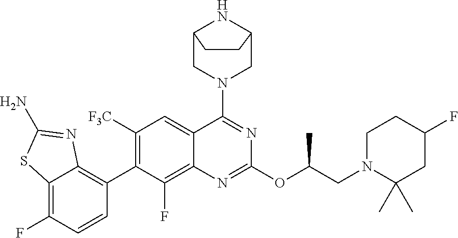 [SMILES: C[C@@H](CN1CCC(F)CC1(C)C)Oc1nc(N2CC3CCC(C2)N3)c2cc(C(F)(F)F)c(-c3ccc(F)c4sc(N)nc34)c(F)c2n1]

E72: 4-(4-(3,8-
diazabicyclo[3.2.1]octan-3-
yl)-8-fluoro-2-(((2S)-1-(4-
fluoro-2,2-dimethylpiperidin-
1-yl)propan-2-yl)oxy)-6-
(trifluoromethyl)quinazolin-7-
yl)-7-fluorobenzo[d]thiazol-2-
amine

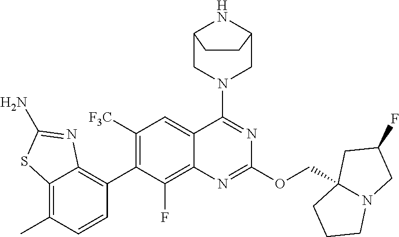 [SMILES: Cc1ccc(-c2c(C(F)(F)F)cc3c(N4CC5CCC(C4)N5)nc(OC[C@@]45CCCN4C[C@H](F)C5)nc3c2F)c2nc(N)sc12]

E73: 4-(4-(3,8-
diazabicyclo[3.2.1]octan-3-yl)-8-
fluoro-2-(((2R,7aS)-2-
fluorotetrahydro-1H-pyrrolizin-
7a(5H)-yl)methoxy)-6-
(trifluoromethyl)quinazolin-7-yl)-
7-methylbenzo[d]thiazol-2-amine

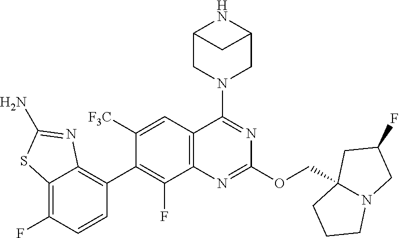 [SMILES: Nc1nc2c(-c3c(C(F)(F)F)cc4c(N5CC6CC(C5)N6)nc(OC[C@@]56CCCN5C[C@H](F)C6)nc4c3F)ccc(F)c2s1]

E74: 4-(4-(3,6-
diazabicyclo[3.1.1]heptan-3-yl)-
8-fluoro-2-(((2R,7aS)-2-
fluorotetrahydro-1H-pyrrolizin-
7a(5H)-yl)methoxy)-6-
(trifluoromethyl)quinazolin-7-
yl)-7-fluorobenzo[d]thiazol-2-
amine

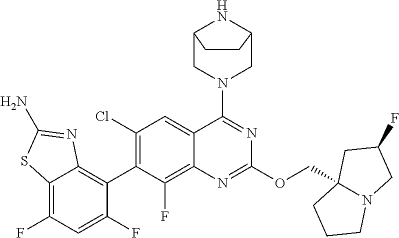 [SMILES: Nc1nc2c(-c3c(Cl)cc4c(N5CC6CCC(C5)N6)nc(OC[C@@]56CCCN5C[C@H](F)C6)nc4c3F)c(F)cc(F)c2s1]

E75: 4-(4-(3,8-
diazabicyclo[3.2.1]octan-3-
yl)-6-chloro-8-fluoro-2-
(((2R,7aS)-2-fluorotetrahydro-
1H-pyrrolizin-7a(5H)-
yl)methoxy)quinazolin-7-yl)-
5,7-difluorobenzo[d]thiazol-2-
amine

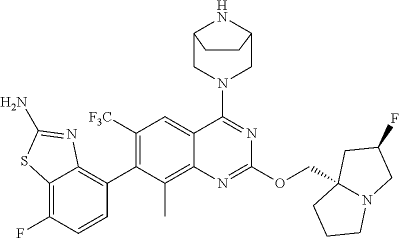 [SMILES: Cc1c(-c2ccc(F)c3sc(N)nc23)c(C(F)(F)F)cc2c(N3CC4CCC(C3)N4)nc(OC[C@@]34CCCN3C[C@H](F)C4)nc12]

E76: 4-(4-(3,8-
diazabicyclo[3.2.1]octan-3-yl)-2-
(((2R,7aS)-2-fluorotetrahydro-
1H-pyrrolizin-7a(5H)-
yl)methoxy)-8-methyl-6-
(trifluoromethyl)quinazolin-7-yl)-
7-fluorobenzo[d]thiazol-2-amine

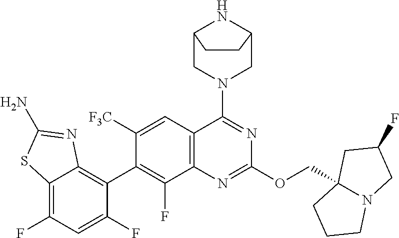 [SMILES: Nc1nc2c(-c3c(C(F)(F)F)cc4c(N5CC6CCC(C5)N6)nc(OC[C@@]56CCCN5C[C@H](F)C6)nc4c3F)c(F)cc(F)c2s1]

T1: 4-(4-(3,8-
diazabicyclo[3.2.1]octan-3-yl)-8-
fluoro-2-(((2R,7aS)-2-
fluorotetrahydro-1H-pyrrolizin-
7a(5H)-yl)methoxy)-6-
(trifluoromethyl)quinazolin-7-
yl)-5,7-difluorobenzo[d]thiazol-
2-amine

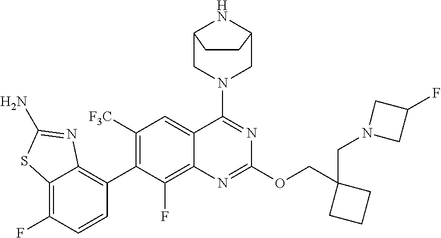 [SMILES: Nc1nc2c(-c3c(C(F)(F)F)cc4c(N5CC6CCC(C5)N6)nc(OCC5(CN6CC(F)C6)CCC5)nc4c3F)ccc(F)c2s1]

T2: 4-(4-(3,8-
diazabicyclo[3.2.1]octan-3-
yl)-8-fluoro-2-((1-((3-
fluoroazetidin-1-
yl)methyl)cyclobutyl)methoxy)-
6-
(trifluoromethyl)quinazolin-7-
yl)-7-fluorobenzo[d]thiazol-2-
amine

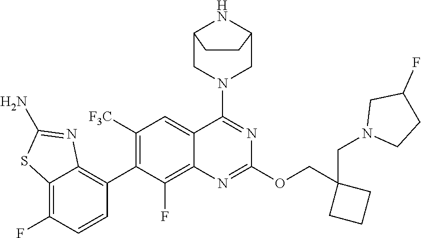 [SMILES: Nc1nc2c(-c3c(C(F)(F)F)cc4c(N5CC6CCC(C5)N6)nc(OCC5(CN6CCC(F)C6)CCC5)nc4c3F)ccc(F)c2s1]

T3: 4-(4-(3,8-
diazabicyclo[3.2.1]octan-3-yl)-8-
fluoro-2-((1-((3-fluoropyrrolidin-
1-
yl)methyl)cyclobutyl)methoxy)-
6-(trifluoromethyl)quinazolin-7-
yl)-7-fluorobenzo[d]thiazol-2-
amine

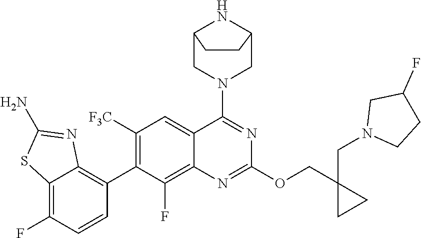 [SMILES: Nc1nc2c(-c3c(C(F)(F)F)cc4c(N5CC6CCC(C5)N6)nc(OCC5(CN6CCC(F)C6)CC5)nc4c3F)ccc(F)c2s1]

T4: 4-(4-(3,8-
diazabicyclo[3.2.1]octan-3-yl)-8-
fluoro-2-((1-((3-fluoropyrrolidin-
1-
yl)methyl)cyclopropyl)methoxy)-
6-(trifluoromethyl)quinazolin-7-
yl)-7-fluorobenzo[d]thiazol-2-
amine

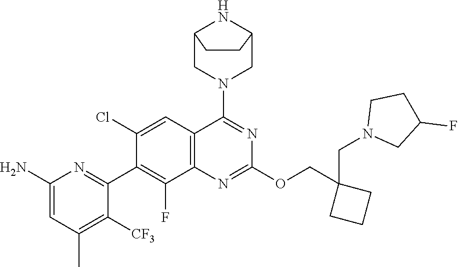 [SMILES: Cc1cc(N)nc(-c2c(Cl)cc3c(N4CC5CCC(C4)N5)nc(OCC4(CN5CCC(F)C5)CCC4)nc3c2F)c1C(F)(F)F]

T5: 6-(4-(3,8-
diazabicyclo[3.2.1]octan-3-
yl)-6-chloro-8-fluoro-2-((1-
((3-fluoropyrrolidin-1-
yl)methyl)cyclobutyl)methoxy)
quinazolin-7-yl)-4-methyl-5-
(trifluoromethyl)pyridin-2-
amine

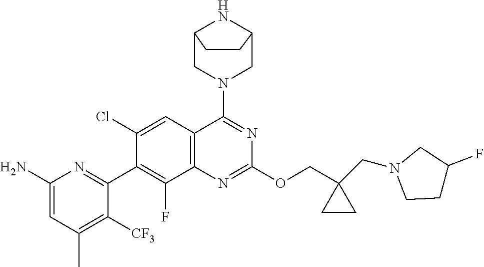 [SMILES: Cc1cc(N)nc(-c2c(Cl)cc3c(N4CC5CCC(C4)N5)nc(OCC4(CN5CCC(F)C5)CC4)nc3c2F)c1C(F)(F)F]

T6: 6-(4-(3,8-
diazabicyclo[3.2.1]octan-3-yl)-6-
chloro-8-fluoro-2-((1-((3-
fluoropyrrolidin-1-
yl)methyl)cyclopropyl)methoxy)
quinazolin-7-yl)-4-methyl-5-
(trifluoromethyl)pyridin-2-amine

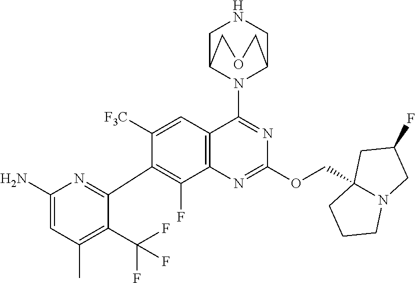 [SMILES: Cc1cc(N)nc(-c2c(C(F)(F)F)cc3c(N4C5CNCC4COC5)nc(OC[C@@]45CCCN4C[C@H](F)C5)nc3c2F)c1C(F)(F)F]

T7: 6-(4-(3-oxa-7,9-
diazabicyclo[3.3.1]nonan-9-yl)-
8-fluoro-2-(((2R,7aS)-2-
fluorotetrahydro-1H-pyrrolizin-
7a(5H)-yl)methoxy)-6-
(trifluoromethyl)quinazolin-7-
yl)-4-methyl-5-
(trifluoromethyl)pyridin-2-amine

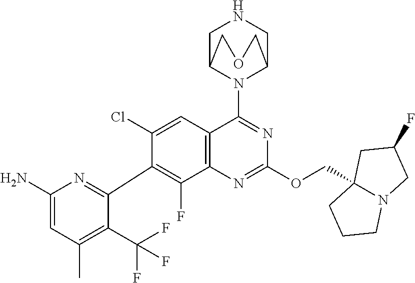 [SMILES: Cc1cc(N)nc(-c2c(Cl)cc3c(N4C5CNCC4COC5)nc(OC[C@@]45CCCN4C[C@H](F)C5)nc3c2F)c1C(F)(F)F]

T8: 6-(4-(3-oxa-7,9-
diazabicyclo[3.3.1]nonan-9-
yl)-6-chloro-8-fluoro-2-
(((2R,7aS)-2-fluorotetrahydro-
1H-pyrrolizin-7a(5H)-
yl)methoxy)quinazolin-7-yl)-
4-methyl-5-
(trifluoromethyl)pyridin-2-
amine

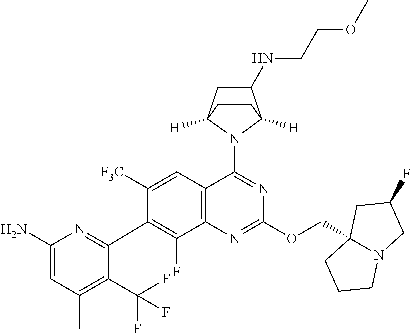 [SMILES: COCCNC1C[C@H]2CC[C@@H]1N2c1nc(OC[C@@]23CCCN2C[C@H](F)C3)nc2c(F)c(-c3nc(N)cc(C)c3C(F)(F)F)c(C(F)(F)F)cc12]

T9: (1S,4R)-7-(7-(6-amino-4-
methyl-3-
(trifluoromethyl)pyridin-2-yl)-8-
fluoro-2-(((2R,7aS)-2-
fluorotetrahydro-1H-pyrrolizin-
7a(5H)-yl)methoxy)-6-
(trifluoromethyl)quinazolin-4-yl)-
N-(2-methoxyethyl)-7-
azabicyclo[2.2.1]heptan-2-amine

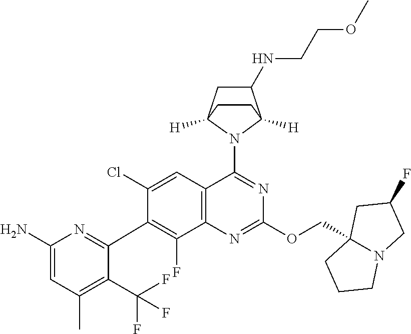 [SMILES: COCCNC1C[C@H]2CC[C@@H]1N2c1nc(OC[C@@]23CCCN2C[C@H](F)C3)nc2c(F)c(-c3nc(N)cc(C)c3C(F)(F)F)c(Cl)cc12]

T10: (1S,4R)-7-(7-(6-amino-4-
methyl-3-
(trifluoromethyl)pyridin-2-yl)-6-
chloro-8-fluoro-2-(((2R,7aS)-2-
fluorotetrahydro-1H-pyrrolizin-
7a(5H)-yl)methoxy)quinazolin-4-
yl)-N-(2-methoxyethyl)-7-
azabicyclo[2.2.1]heptan-2-amine

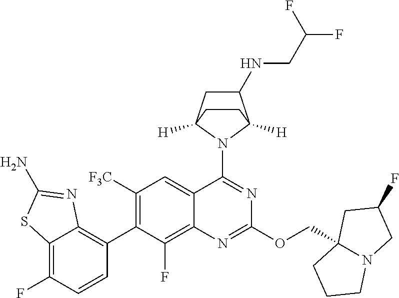 [SMILES: Nc1nc2c(-c3c(C(F)(F)F)cc4c(N5[C@@H]6CC[C@H]5C(NCC(F)F)C6)nc(OC[C@@]56CCCN5C[C@H](F)C6)nc4c3F)ccc(F)c2s1]

T11: 4-(4-((1S,4R)-2-((2,2-
difluoroethyl)amino)-7-
azabicyclo[2.2.1]heptan-7-yl)-
8-fluoro-2-(((2R,7aS)-2-
fluorotetrahydro-1H-
pyrrolizin-7a(5H)-
yl)methoxy)-6-
(trifluoromethyl)quinazolin-7-
yl)-7-fluorobenzo[d]thiazol-2-
amine

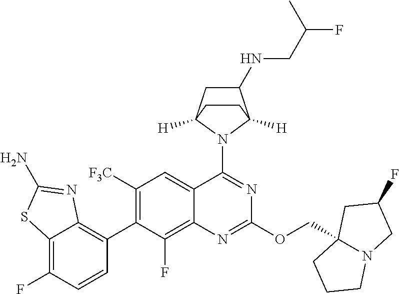 [SMILES: CC(F)CNC1C[C@H]2CC[C@@H]1N2c1nc(OC[C@@]23CCCN2C[C@H](F)C3)nc2c(F)c(-c3ccc(F)c4sc(N)nc34)c(C(F)(F)F)cc12]

T12: 7-fluoro-4-(8-fluoro-4-
((1S,4R)-2-((2-
fluoropropyl)amino)-7-
azabicyclo[2.2.1]heptan-7-yl)-2-
(((2R,7aS)-2-fluorotetrahydro-
1H-pyrrolizin-7a(5H)-
yl)methoxy)-6-
(trifluoromethyl)quinazolin-7-
yl)benzo[d]thiazol-2-amine

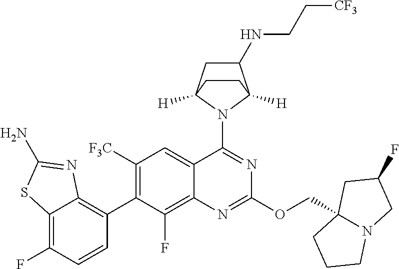 [SMILES: Nc1nc2c(-c3c(C(F)(F)F)cc4c(N5[C@@H]6CC[C@H]5C(NCCC(F)(F)F)C6)nc(OC[C@@]56CCCN5C[C@H](F)C6)nc4c3F)ccc(F)c2s1]

T13: 7-fluoro-4-(8-fluoro-2-
(((2R,7aS)-2-fluorotetrahydro-
1H-pyrrolizin-7a(5H)-
yl)methoxy)-6-(trifluoromethyl)-
4-((1S,4R)-2-((3,3,3-
trifluoropropyl)amino)-7-
azabicyclo[2.2.1]heptan-7-
yl)quinazolin-7-
yl)benzo[d]thiazol-2-amine

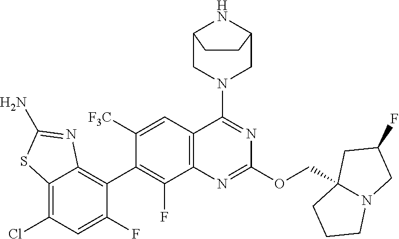 [SMILES: Nc1nc2c(-c3c(C(F)(F)F)cc4c(N5CC6CCC(C5)N6)nc(OC[C@@]56CCCN5C[C@H](F)C6)nc4c3F)c(F)cc(Cl)c2s1]

T14: 4-(4-(3,8-
diazabicyclo[3.2.1]octan-3-
yl)-8-fluoro-2-(((2R,7aS)-2-
fluorotetrahydro-1H-
pyrrolizin-7a(5H)-
yl)methoxy)-6-
(trifluoromethyl)quinazolin-7-
yl)-7-chloro-5-
fluorobenzo[d]thiazol-2-amine

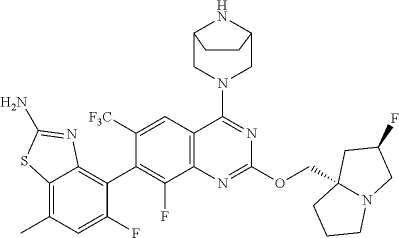 [SMILES: Cc1cc(F)c(-c2c(C(F)(F)F)cc3c(N4CC5CCC(C4)N5)nc(OC[C@@]45CCCN4C[C@H](F)C5)nc3c2F)c2nc(N)sc12]

T15: 4-(4-(3,8-
diazabicyclo[3.2.1]octan-3-yl)-8-
fluoro-2-(((2R,7aS)-2-
fluorotetrahydro-1H-pyrrolizin-
7a(5H)-yl)methoxy)-6-
(trifluoromethyl)quinazolin-7-yl)-
5-fluoro-7-
methylbenzo[d]thiazol-2-amine

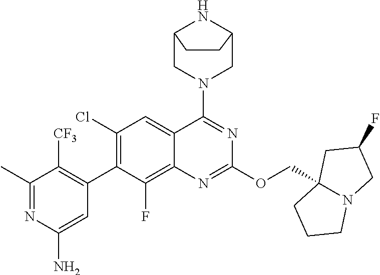 [SMILES: Cc1nc(N)cc(-c2c(Cl)cc3c(N4CC5CCC(C4)N5)nc(OC[C@@]45CCCN4C[C@H](F)C5)nc3c2F)c1C(F)(F)F]

T16: 4-(4-(3,8-
diazabicyclo[3.2.1]octan-3-yl)-6-
chloro-8-fluoro-2-(((2R,7aS)-2-
fluorotetrahydro-1H-pyrrolizin-
7a(5H)-yl)methoxy)quinazolin-7-
yl)-6-methyl-5-
(trifluoromethyl)pyridin-2-amine

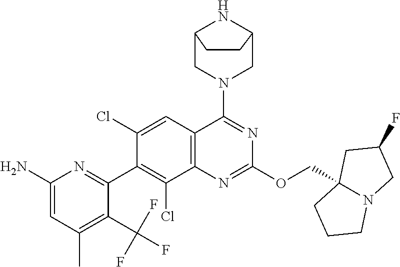 [SMILES: Cc1cc(N)nc(-c2c(Cl)cc3c(N4CC5CCC(C4)N5)nc(OC[C@@]45CCCN4C[C@H](F)C5)nc3c2Cl)c1C(F)(F)F]

T17: 6-(4-(3,8-
diazabicyclo[3.2.1]octan-3-
yl)-6,8-dichloro-2-(((2R,7aS)-
2-fluorotetrahydro-1H-
pyrrolizin-7a(5H)-
yl)methoxy)quinazolin-7-yl)-
4-methyl-5-
(trifluoromethyl)pyridin-2-
amine

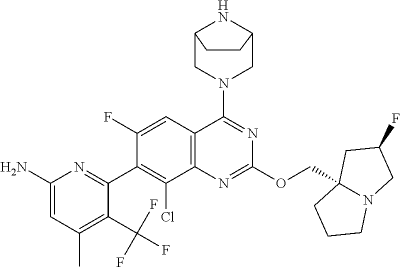 [SMILES: Cc1cc(N)nc(-c2c(F)cc3c(N4CC5CCC(C4)N5)nc(OC[C@@]45CCCN4C[C@H](F)C5)nc3c2Cl)c1C(F)(F)F]

T18: 6-(4-(3,8-
diazabicyclo[3.2.1]octan-3-yl)-8-
chloro-6-fluoro-2-(((2R,7aS)-2-
fluorotetrahydro-1H-pyrrolizin-
7a(5H)-yl)methoxy)quinazolin-7-
yl)-4-methyl-5-
(trifluoromethyl)pyridin-2-amine

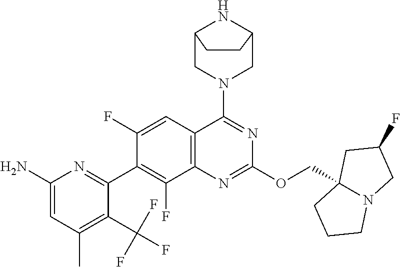 [SMILES: Cc1cc(N)nc(-c2c(F)cc3c(N4CC5CCC(C4)N5)nc(OC[C@@]45CCCN4C[C@H](F)C5)nc3c2F)c1C(F)(F)F]

T19: 6-(4-(3,8-
diazabicyclo[3.2.1]octan-3-yl)-
6,8-difluoro-2-(((2R,7aS)-2-
fluorotetrahydro-1H-pyrrolizin-
7a(5H)-yl)methoxy)quinazolin-7-
yl)-4-methyl-5-
(trifluoromethyl)pyridin-2-amine

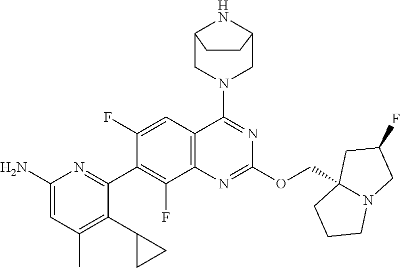 [SMILES: Cc1cc(N)nc(-c2c(F)cc3c(N4CC5CCC(C4)N5)nc(OC[C@@]45CCCN4C[C@H](F)C5)nc3c2F)c1C1CC1]

T20: 6-(4-(3,8-
diazabicyclo[3.2.1]octan-3-
yl)-6,8-difluoro-2-(((2R,7aS)-
2-fluorotetrahydro-1H-
pyrrolizin-7a(5H)-
yl)methoxy)quinazolin-7-yl)-
5-cyclopropyl-4-
methylpyridin-2-amine

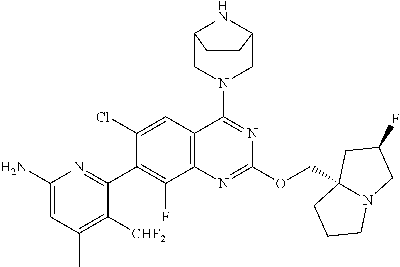 [SMILES: Cc1cc(N)nc(-c2c(Cl)cc3c(N4CC5CCC(C4)N5)nc(OC[C@@]45CCCN4C[C@H](F)C5)nc3c2F)c1C(F)F]

T21: 6-(4-(3,8-
diazabicyclo[3.2.1]octan-3-yl)-6-
chloro-8-fluoro-2-(((2R,7aS)-2-
fluorotetrahydro-1H-pyrrolizin-
7a(5H)-yl)methoxy)quinazolin-7-
yl)-5-(difluoromethyl)-4-
methylpyridin-2-amine

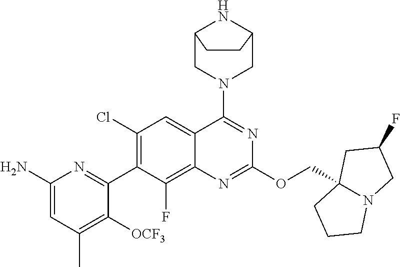 [SMILES: Cc1cc(N)nc(-c2c(Cl)cc3c(N4CC5CCC(C4)N5)nc(OC[C@@]45CCCN4C[C@H](F)C5)nc3c2F)c1OC(F)(F)F]

T22: 6-(4-(3,8-
diazabicyclo[3.2.1]octan-3-yl)-6-
chloro-8-fluoro-2-(((2R,7aS)-2-
fluorotetrahydro-1H-pyrrolizin-
7a(5H)-yl)methoxy)quinazolin-7-
yl)-4-methyl-5-
(trifluoromethoxy)pyridin-2-
amine

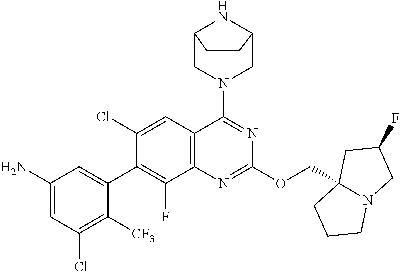 [SMILES: Nc1cc(Cl)c(C(F)(F)F)c(-c2c(Cl)cc3c(N4CC5CCC(C4)N5)nc(OC[C@@]45CCCN4C[C@H](F)C5)nc3c2F)c1]

T23: 3-(4-(3,8-
diazabicyclo[3.2.1]octan-3-
yl)-6-chloro-8-fluoro-2-
(((2R,7aS)-2-fluorotetrahydro-
1H-pyrrolizin-7a(5H)-
yl)methoxy)quinazolin-7-yl)-
5-chloro-4-
(trifluoromethyl)aniline

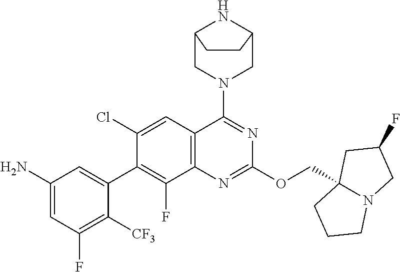 [SMILES: Nc1cc(F)c(C(F)(F)F)c(-c2c(Cl)cc3c(N4CC5CCC(C4)N5)nc(OC[C@@]45CCCN4C[C@H](F)C5)nc3c2F)c1]

T24: 3-(4-(3,8-
diazabicyclo[3.2.1]octan-3-yl)-6-
chloro-8-fluoro-2-(((2R,7aS)-2-
fluorotetrahydro-1H-pyrrolizin-
7a(5H)-yl)methoxy)quinazolin-7-
yl)-5-fluoro-4-
(trifluoromethyl)aniline

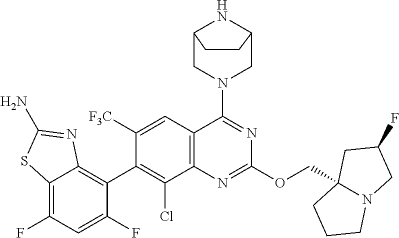 [SMILES: Nc1nc2c(-c3c(C(F)(F)F)cc4c(N5CC6CCC(C5)N6)nc(OC[C@@]56CCCN5C[C@H](F)C6)nc4c3Cl)c(F)cc(F)c2s1]

T25: 4-(4-(3,8-
diazabicyclo[3.2.1]octan-3-yl)-8-
chloro-2-(((2R,7aS)-2-
fluorotetrahydro-1H-pyrrolizin-
7a(5H)-yl)methoxy)-6-
(trifluoromethyl)quinazolin-7-
yl)-5,7-difluorobenzo[d]thiazol-
2-amine

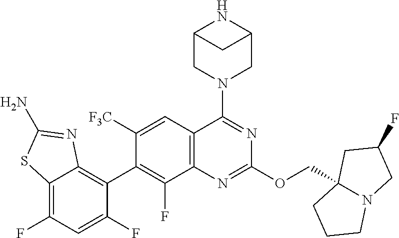 [SMILES: Nc1nc2c(-c3c(C(F)(F)F)cc4c(N5CC6CC(C5)N6)nc(OC[C@@]56CCCN5C[C@H](F)C6)nc4c3F)c(F)cc(F)c2s1]

T26: 4-(4-(3,6-
diazabicyclo[3.1.1]heptan-3-
yl)-8-fluoro-2-(((2R,7aS)-2-
fluorotetrahydro-1H-
pyrrolizin-7a(5H)-
yl)methoxy)-6-
(trifluoromethyl)quinazolin-7-
yl)-5,7-
difluorobenzo[d]thiazol-2-
amine

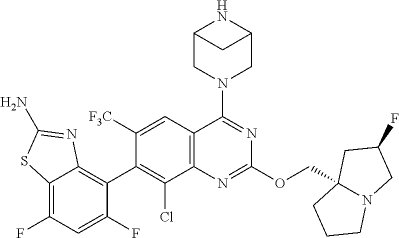 [SMILES: Nc1nc2c(-c3c(C(F)(F)F)cc4c(N5CC6CC(C5)N6)nc(OC[C@@]56CCCN5C[C@H](F)C6)nc4c3Cl)c(F)cc(F)c2s1]

T27: 4-(4-(3,6-
diazabicyclo[3.1.1]heptan-3-yl)-
8-chloro-2-(((2R,7aS)-2-
fluorotetrahydro-1H-pyrrolizin-
7a(5H)-yl)methoxy)-6-
(trifluoromethyl)quinazolin-7-yl)-
5,7-difluorobenzo[d]thiazol-2-
amine

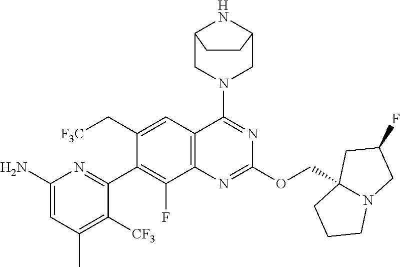 [SMILES: Cc1cc(N)nc(-c2c(CC(F)(F)F)cc3c(N4CC5CCC(C4)N5)nc(OC[C@@]45CCCN4C[C@H](F)C5)nc3c2F)c1C(F)(F)F]

T28: 6-(4-(3,8-
diazabicyclo[3.2.1]octan-3-yl)-8-
fluoro-2-(((2R,7aS)-2-
fluorotetrahydro-1H-pyrrolizin-
7a(5H)-yl)methoxy)-6-(2,2,2-
trifluoroethyl)quinazolin-7-yl)-4-
methyl-5-
(trifluoromethyl)pyridin-2-amine

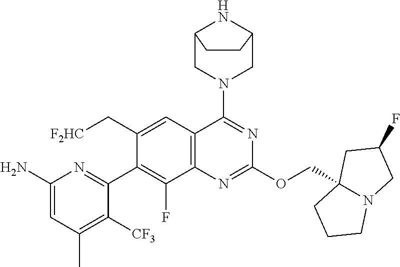 [SMILES: Cc1cc(N)nc(-c2c(CC(F)F)cc3c(N4CC5CCC(C4)N5)nc(OC[C@@]45CCCN4C[C@H](F)C5)nc3c2F)c1C(F)(F)F]

T29: 6-(4-(3,8-
diazabicyclo[3.2.1]octan-3-
yl)-6-(2,2-difluoroethyl)-8-
fluoro-2-(((2R,7aS)-2-
fluorotetrahydro-1H-
pyrrolizin-7a(5H)-
yl)methoxy)quinazolin-7-yl)-
4-methyl-5-
(trifluoromethyl)pyridin-2-
amine

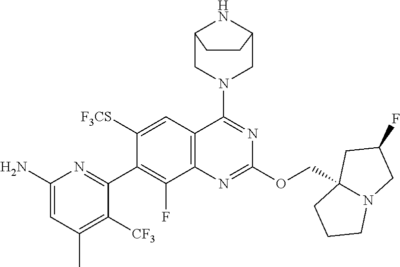

T30: 6-(4-(3,8-
diazabicyclo[3.2.1]octan-3-yl)-8-
fluoro-2-(((2R,7aS)-2-
fluorotetrahydro-1H-pyrrolizin-
7a(5H)-yl)methoxy)-6-
((trifluoromethyl)thio)quinazolin-
7-yl)-4-methyl-5-
(trifluoromethyl)pyridin-2-amine

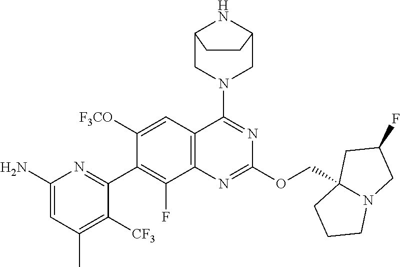 [SMILES: Cc1cc(N)nc(-c2c(OC(F)(F)F)cc3c(N4CC5CCC(C4)N5)nc(OC[C@@]45CCCN4C[C@H](F)C5)nc3c2F)c1C(F)(F)F]

T31: 6-(4-(3,8-
diazabicyclo[3.2.1]octan-3-yl)-8-
fluoro-2-(((2R,7aS)-2-
fluorotetrahydro-1H-pyrrolizin-
7a(5H)-yl)methoxy)-6-
(trifluoromethoxy)quinazolin-7-
yl)-4-methyl-5-
(trifluoromethyl)pyridin-2-amine

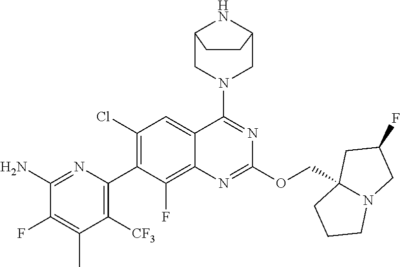 [SMILES: Cc1c(F)c(N)nc(-c2c(Cl)cc3c(N4CC5CCC(C4)N5)nc(OC[C@@]45CCCN4C[C@H](F)C5)nc3c2F)c1C(F)(F)F]

T32: 6-(4-(3,8-
diazabicyclo[3.2.1]octan-3-
yl)-6-chloro-8-fluoro-2-
(((2R,7aS)-2-fluorotetrahydro-
1H-pyrrolizin-7a(5H)-
yl)methoxy)quinazolin-7-yl)-
3-fluoro-4-methyl-5-
(trifluoromethyl)pyridin-2-
amine

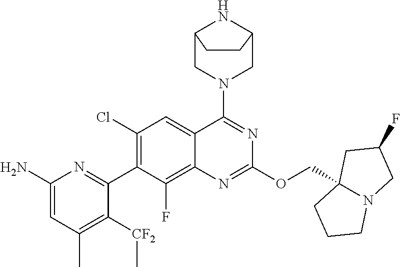 [SMILES: Cc1cc(N)nc(-c2c(Cl)cc3c(N4CC5CCC(C4)N5)nc(OC[C@@]45CCCN4C[C@H](F)C5)nc3c2F)c1C(C)(F)F]

T33: 6-(4-(3,8-
diazabicyclo[3.2.1]octan-3-yl)-6-
chloro-8-fluoro-2-(((2R,7aS)-2-
fluorotetrahydro-1H-pyrrolizin-
7a(5H)-yl)methoxy)quinazolin-7-
yl)-5-(1,1-difluoroethyl)-4-
methylpyridin-2-amine

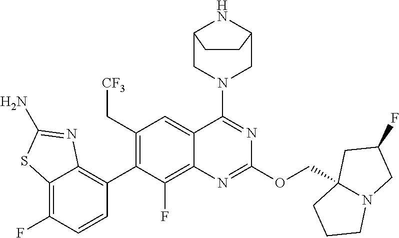 [SMILES: Nc1nc2c(-c3c(CC(F)(F)F)cc4c(N5CC6CCC(C5)N6)nc(OC[C@@]56CCCN5C[C@H](F)C6)nc4c3F)ccc(F)c2s1]

T34: 4-(4-(3,8-
diazabicyclo[3.2.1]octan-3-yl)-8-
fluoro-2-(((2R, 7aS)-2-
fluorotetrahydro-1H-pyrrolizin-
7a(5H)-yl)methoxy)-6-(2,2,2-
trifluoroethyl)quinazolin-7-yl)-7-
fluorobenzo[d]thiazol-2-amine

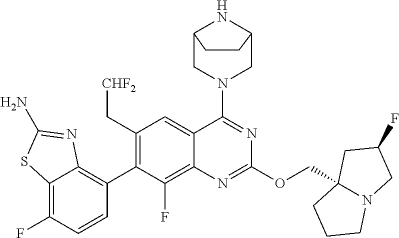 [SMILES: Nc1nc2c(-c3c(CC(F)F)cc4c(N5CC6CCC(C5)N6)nc(OC[C@@]56CCCN5C[C@H](F)C6)nc4c3F)ccc(F)c2s1]

T35: 4-(4-(3,8-
diazabicyclo[3.2.1]octan-3-
yl)-6-(2,2-difluoroethyl)-8-
fluoro-2-(((2R,7aS)-2-
fluorotetrahydro-1H-
pyrrolizin-7a(5H)-
yl)methoxy)quinazolin-7-yl)-
7-fluorobenzo[d]thiazol-2-
amine

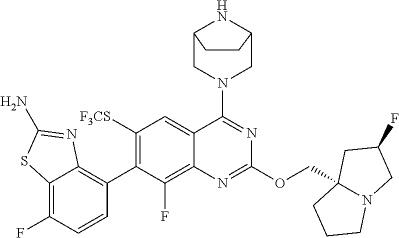

T36: 4-(4-(3,8-
diazabicyclo[3.2.1]octan-3-yl)-8-
fluoro-2-(((2R,7aS)-2-
fluorotetrahydro-1H-pyrrolizin-
7a(5H)-yl)methoxy)-6-
((trifluoromethyl)thio)quinazolin-
7-yl)-7-fluorobenzo[d]thiazol-2-
amine

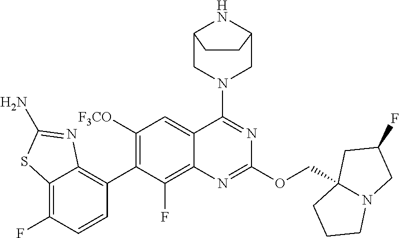 [SMILES: Nc1nc2c(-c3c(OC(F)(F)F)cc4c(N5CC6CCC(C5)N6)nc(OC[C@@]56CCCN5C[C@H](F)C6)nc4c3F)ccc(F)c2s1]

T37: 4-(4-(3,8-
diazabicyclo[3.2.1]octan-3-yl)-8-
fluoro-2-(((2R,7aS)-2-
fluorotetrahydro-1H-pyrrolizin-
7a(5H)-yl)methoxy)-6-
(trifluoromethoxy)quinazolin-7-
yl)-7-fluorobenzo[d]thiazol-2-
amine

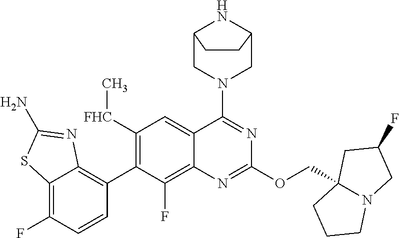

T38: 4-(4-(3,8-
diazabicyclo[3.2.1]octan-3-
yl)-8-fluoro-6-(1-fluoroethyl)-
2-(((2R,7aS)-2-
fluorotetrahydro-1H-
pyrrolizin-7a(5H)-
yl)methoxy)quinazolin-7-yl)-
7-fluorobenzo[d]thiazol-2-
amine

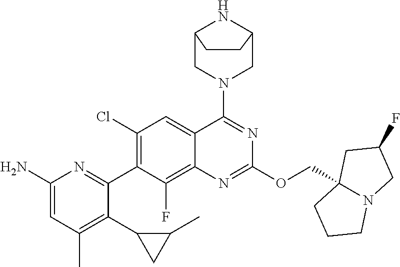 [SMILES: Cc1cc(N)nc(-c2c(Cl)cc3c(N4CC5CCC(C4)N5)nc(OC[C@@]45CCCN4C[C@H](F)C5)nc3c2F)c1C1CC1C]

T39: 6-(4-(3,8-
diazabicyclo[3.2.1]octan-3-yl)-6-
chloro-8-fluoro-2-(((2R,7aS)-2-
fluorotetrahydro-1H-pyrrolizin-
7a(5H)-yl)methoxy)quinazolin-7-
yl)-4-methyl-5-(2-
methylcyclopropyl)pyridin-2-
amine

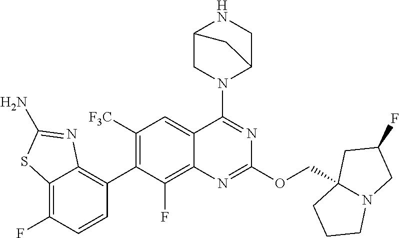 [SMILES: Nc1nc2c(-c3c(C(F)(F)F)cc4c(N5CC6CC5CN6)nc(OC[C@@]56CCCN5C[C@H](F)C6)nc4c3F)ccc(F)c2s1]

T40: 4-(4-(2,5-
diazabicyclo[2.2.1]heptan-2-yl)-
8-fluoro-2-(((2R,7aS)-2-
fluorotetrahydro-1H-pyrrolizin-
7a(5H)-yl)methoxy)-6-
(trifluoromethyl)quinazolin-7-
yl)-7-fluorobenzo[d]thiazol-2-
amine

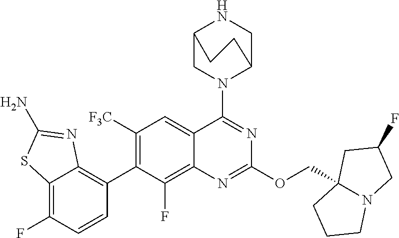 [SMILES: Nc1nc2c(-c3c(C(F)(F)F)cc4c(N5CC6CCC5CN6)nc(OC[C@@]56CCCN5C[C@H](F)C6)nc4c3F)ccc(F)c2s1]

T41: 4-(4-(2,5-
diazabicyclo[2.2.2]octan-2-
yl)-8-fluoro-2-(((2R,7aS)-2-
fluorotetrahydro-1H-
pyrrolizin-7a(5H)-
yl)methoxy)-6-
(trifluoromethyl)quinazolin-7-
yl)-7-fluorobenzo[d]thiazol-2-
amine

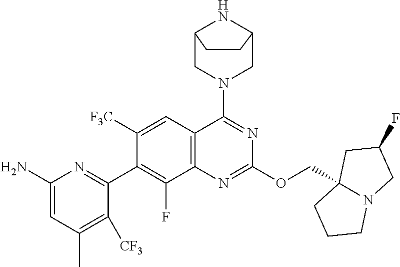 [SMILES: Cc1cc(N)nc(-c2c(C(F)(F)F)cc3c(N4CC5CCC(C4)N5)nc(OC[C@@]45CCCN4C[C@H](F)C5)nc3c2F)c1C(F)(F)F]

E77 (Final product of Example
77)

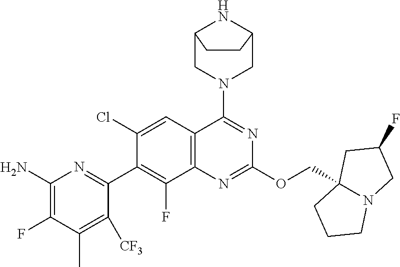 [SMILES: Cc1c(F)c(N)nc(-c2c(Cl)cc3c(N4CC5CCC(C4)N5)nc(OC[C@@]45CCCN4C[C@H](F)C5)nc3c2F)c1C(F)(F)F]

E78

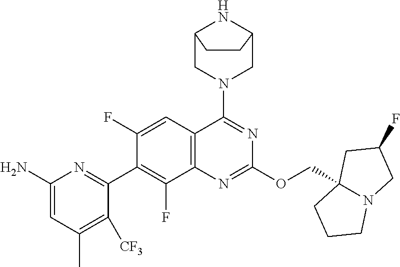 [SMILES: Cc1cc(N)nc(-c2c(F)cc3c(N4CC5CCC(C4)N5)nc(OC[C@@]45CCCN4C[C@H](F)C5)nc3c2F)c1C(F)(F)F]

E79

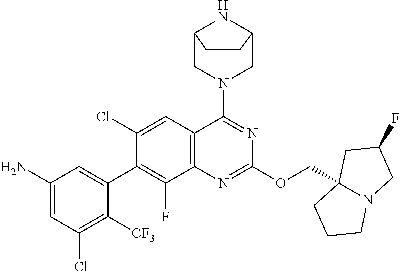 [SMILES: Nc1cc(Cl)c(C(F)(F)F)c(-c2c(Cl)cc3c(N4CC5CCC(C4)N5)nc(OC[C@@]45CCCN4C[C@H](F)C5)nc3c2F)c1]

E80

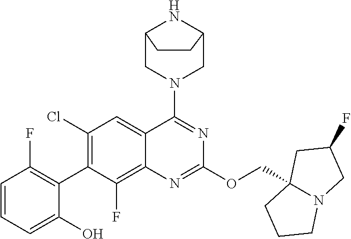 [SMILES: Oc1cccc(F)c1-c1c(Cl)cc2c(N3CC4CCC(C3)N4)nc(OC[C@@]34CCCN3C[C@H](F)C4)nc2c1F]

E81

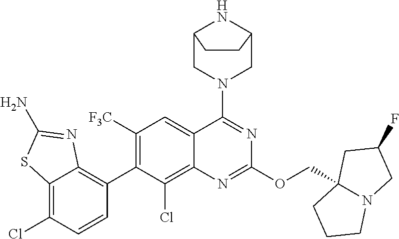 [SMILES: Nc1nc2c(-c3c(C(F)(F)F)cc4c(N5CC6CCC(C5)N6)nc(OC[C@@]56CCCN5C[C@H](F)C6)nc4c3Cl)ccc(Cl)c2s1]

E82

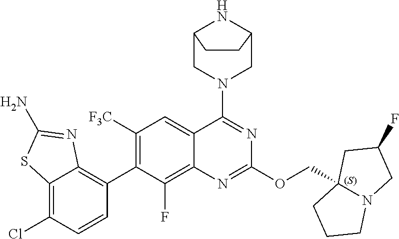

E83

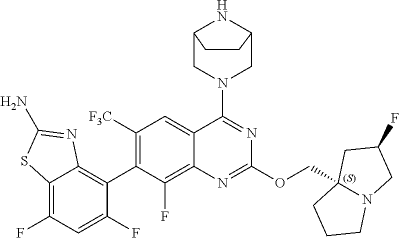

E84

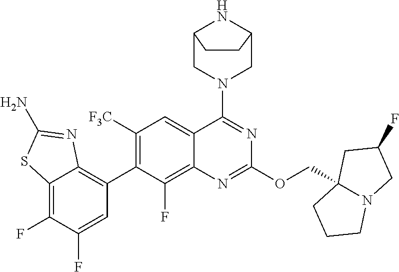 [SMILES: Nc1nc2c(-c3c(C(F)(F)F)cc4c(N5CC6CCC(C5)N6)nc(OC[C@@]56CCCN5C[C@H](F)C6)nc4c3F)cc(F)c(F)c2s1]

E85

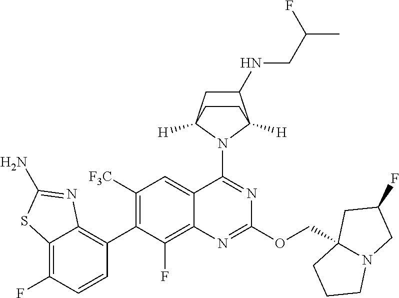 [SMILES: CC(F)CNC1C[C@H]2CC[C@@H]1N2c1nc(OC[C@@]23CCCN2C[C@H](F)C3)nc2c(F)c(-c3ccc(F)c4sc(N)nc34)c(C(F)(F)F)cc12]

E86

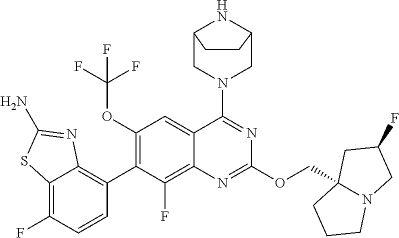 [SMILES: Nc1nc2c(-c3c(OC(F)(F)F)cc4c(N5CC6CCC(C5)N6)nc(OC[C@@]56CCCN5C[C@H](F)C6)nc4c3F)ccc(F)c2s1]

E87

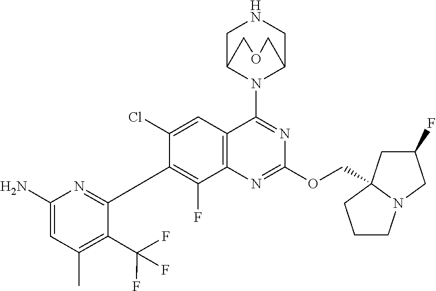 [SMILES: Cc1cc(N)nc(-c2c(Cl)cc3c(N4C5CNCC4COC5)nc(OC[C@@]45CCCN4C[C@H](F)C5)nc3c2F)c1C(F)(F)F]

E88

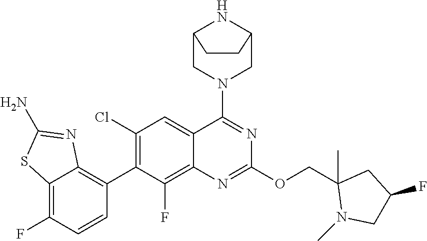 [SMILES: CN1C[C@H](F)CC1(C)COc1nc(N2CC3CCC(C2)N3)c2cc(Cl)c(-c3ccc(F)c4sc(N)nc34)c(F)c2n1]

E89

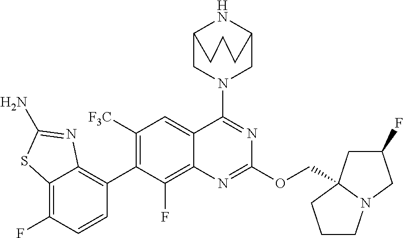 [SMILES: Nc1nc2c(-c3c(C(F)(F)F)cc4c(N5CC6CCCC(C5)N6)nc(OC[C@@]56CCCN5C[C@H](F)C6)nc4c3F)ccc(F)c2s1]

E90

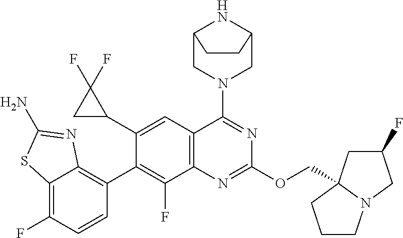 [SMILES: Nc1nc2c(-c3c(C4CC4(F)F)cc4c(N5CC6CCC(C5)N6)nc(OC[C@@]56CCCN5C[C@H](F)C6)nc4c3F)ccc(F)c2s1]

E91

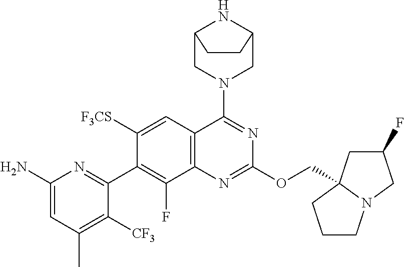

E92

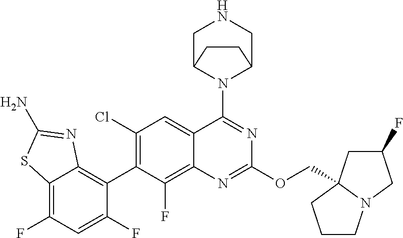 [SMILES: Nc1nc2c(-c3c(Cl)cc4c(N5C6CCC5CNC6)nc(OC[C@@]56CCCN5C[C@H](F)C6)nc4c3F)c(F)cc(F)c2s1]

E93

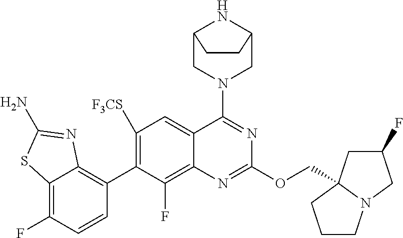

E94

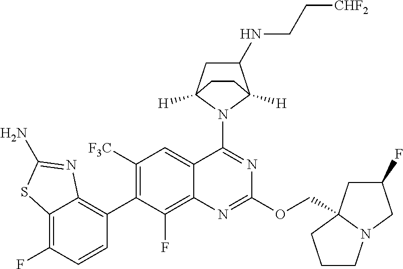 [SMILES: Nc1nc2c(-c3c(C(F)(F)F)cc4c(N5[C@@H]6CC[C@H]5C(NCCC(F)F)C6)nc(OC[C@@]56CCCN5C[C@H](F)C6)nc4c3F)ccc(F)c2s1]

E95

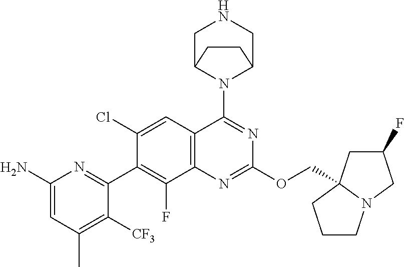 [SMILES: Cc1cc(N)nc(-c2c(Cl)cc3c(N4C5CCC4CNC5)nc(OC[C@@]45CCCN4C[C@H](F)C5)nc3c2F)c1C(F)(F)F]

E96

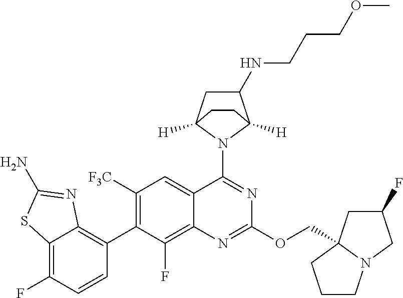 [SMILES: COCCCNC1C[C@H]2CC[C@@H]1N2c1nc(OC[C@@]23CCCN2C[C@H](F)C3)nc2c(F)c(-c3ccc(F)c4sc(N)nc34)c(C(F)(F)F)cc12]

E97

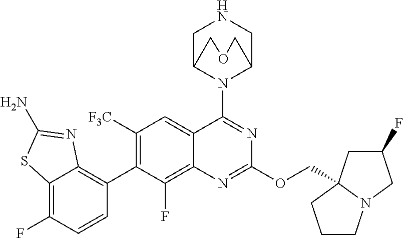 [SMILES: Nc1nc2c(-c3c(C(F)(F)F)cc4c(N5C6CNCC5COC6)nc(OC[C@@]56CCCN5C[C@H](F)C6)nc4c3F)ccc(F)c2s1]

E98

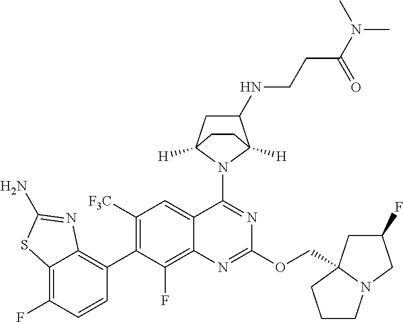 [SMILES: CN(C)C(=O)CCNC1C[C@H]2CC[C@@H]1N2c1nc(OC[C@@]23CCCN2C[C@H](F)C3)nc2c(F)c(-c3ccc(F)c4sc(N)nc34)c(C(F)(F)F)cc12]

E99

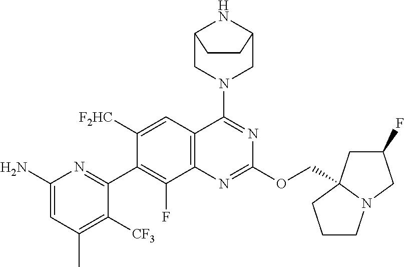 [SMILES: Cc1cc(N)nc(-c2c(C(F)F)cc3c(N4CC5CCC(C4)N5)nc(OC[C@@]45CCCN4C[C@H](F)C5)nc3c2F)c1C(F)(F)F]

E100

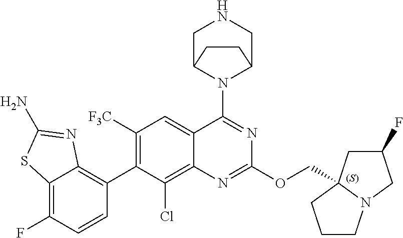

E101

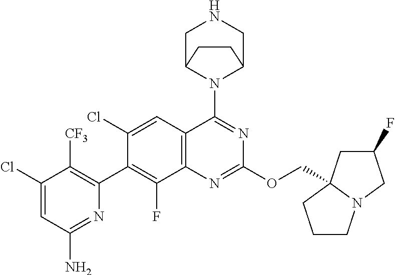 [SMILES: Nc1cc(Cl)c(C(F)(F)F)c(-c2c(Cl)cc3c(N4C5CCC4CNC5)nc(OC[C@@]45CCCN4C[C@H](F)C5)nc3c2F)n1]

E102

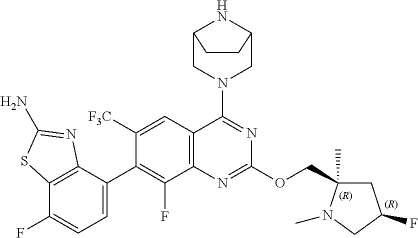

E103

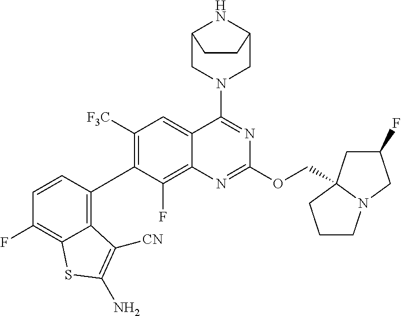 [SMILES: N#Cc1c(N)sc2c(F)ccc(-c3c(C(F)(F)F)cc4c(N5CC6CCC(C5)N6)nc(OC[C@@]56CCCN5C[C@H](F)C6)nc4c3F)c12]

E104

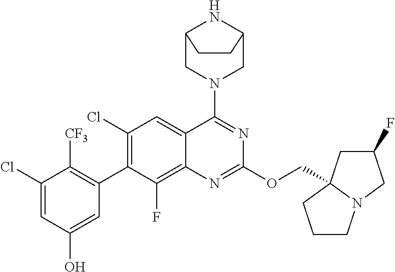 [SMILES: Oc1cc(Cl)c(C(F)(F)F)c(-c2c(Cl)cc3c(N4CC5CCC(C4)N5)nc(OC[C@@]45CCCN4C[C@H](F)C5)nc3c2F)c1]

E105

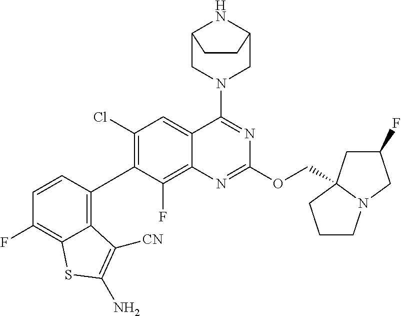 [SMILES: N#Cc1c(N)sc2c(F)ccc(-c3c(Cl)cc4c(N5CC6CCC(C5)N6)nc(OC[C@@]56CCCN5C[C@H](F)C6)nc4c3F)c12]

E106

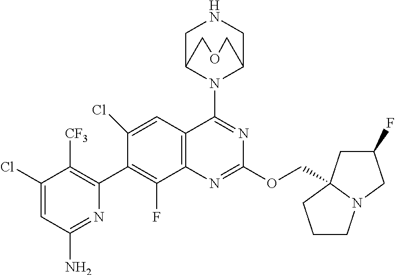 [SMILES: Nc1cc(Cl)c(C(F)(F)F)c(-c2c(Cl)cc3c(N4C5CNCC4COC5)nc(OC[C@@]45CCCN4C[C@H](F)C5)nc3c2F)n1]

E107

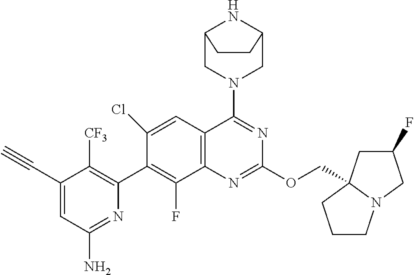 [SMILES: C#Cc1cc(N)nc(-c2c(Cl)cc3c(N4CC5CCC(C4)N5)nc(OC[C@@]45CCCN4C[C@H](F)C5)nc3c2F)c1C(F)(F)F]

E108

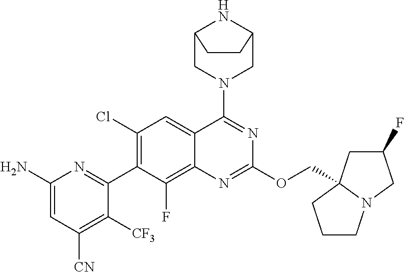 [SMILES: N#Cc1cc(N)nc(-c2c(Cl)cc3c(N4CC5CCC(C4)N5)nc(OC[C@@]45CCCN4C[C@H](F)C5)nc3c2F)c1C(F)(F)F]

E109

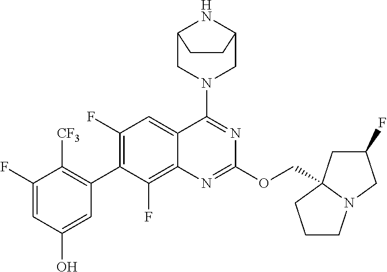 [SMILES: Oc1cc(F)c(C(F)(F)F)c(-c2c(F)cc3c(N4CC5CCC(C4)N5)nc(OC[C@@]45CCCN4C[C@H](F)C5)nc3c2F)c1]

E110

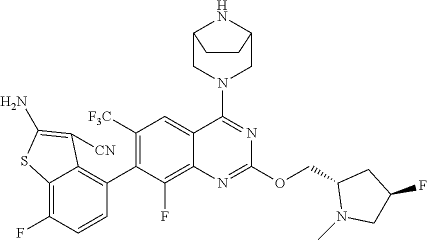 [SMILES: CN1C[C@H](F)C[C@H]1COc1nc(N2CC3CCC(C2)N3)c2cc(C(F)(F)F)c(-c3ccc(F)c4sc(N)c(C#N)c34)c(F)c2n1]

E111

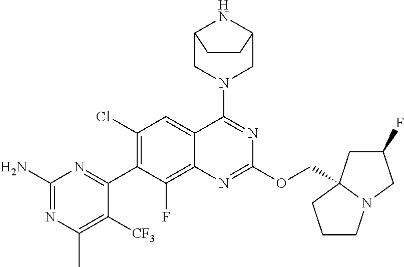 [SMILES: Cc1nc(N)nc(-c2c(Cl)cc3c(N4CC5CCC(C4)N5)nc(OC[C@@]45CCCN4C[C@H](F)C5)nc3c2F)c1C(F)(F)F]

E112

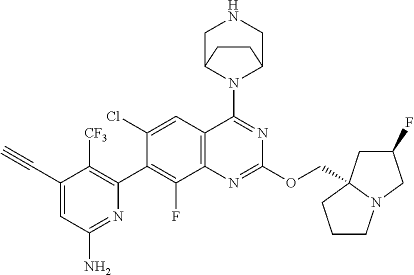 [SMILES: C#Cc1cc(N)nc(-c2c(Cl)cc3c(N4C5CCC4CNC5)nc(OC[C@@]45CCCN4C[C@H](F)C5)nc3c2F)c1C(F)(F)F]

E113

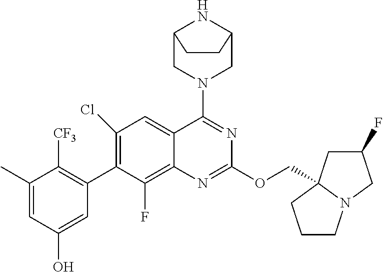 [SMILES: Cc1cc(O)cc(-c2c(Cl)cc3c(N4CC5CCC(C4)N5)nc(OC[C@@]45CCCN4C[C@H](F)C5)nc3c2F)c1C(F)(F)F]

E114

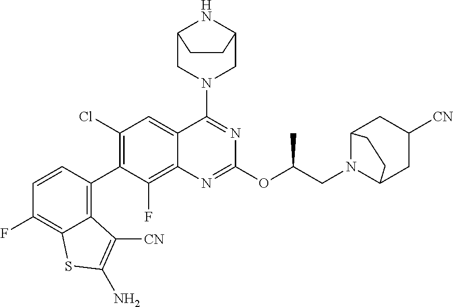 [SMILES: C[C@@H](CN1C2CCC1CC(C#N)C2)Oc1nc(N2CC3CCC(C2)N3)c2cc(Cl)c(-c3ccc(F)c4sc(N)c(C#N)c34)c(F)c2n1]

E115

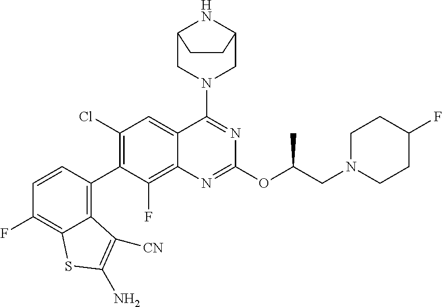 [SMILES: C[C@@H](CN1CCC(F)CC1)Oc1nc(N2CC3CCC(C2)N3)c2cc(Cl)c(-c3ccc(F)c4sc(N)c(C#N)c34)c(F)c2n1]

E116

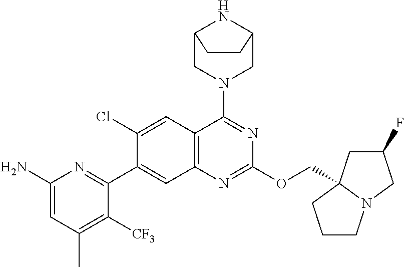 [SMILES: Cc1cc(N)nc(-c2cc3nc(OC[C@@]45CCCN4C[C@H](F)C5)nc(N4CC5CCC(C4)N5)c3cc2Cl)c1C(F)(F)F]

E117

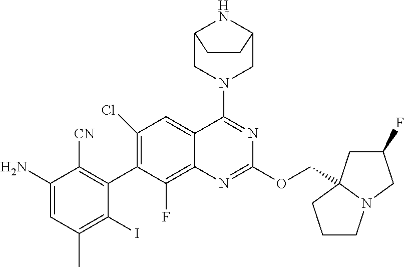

E118

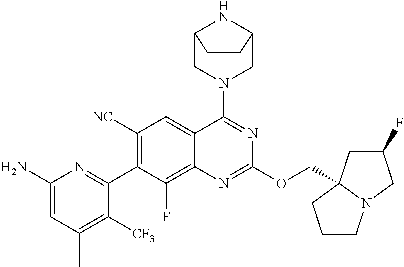 [SMILES: Cc1cc(N)nc(-c2c(C#N)cc3c(N4CC5CCC(C4)N5)nc(OC[C@@]45CCCN4C[C@H](F)C5)nc3c2F)c1C(F)(F)F]

E119

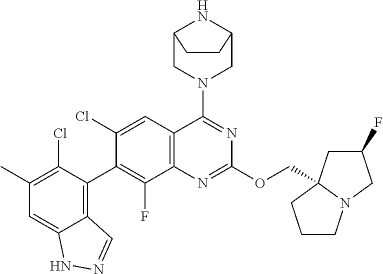 [SMILES: Cc1cc2[nH]ncc2c(-c2c(Cl)cc3c(N4CC5CCC(C4)N5)nc(OC[C@@]45CCCN4C[C@H](F)C5)nc3c2F)c1Cl]

E120

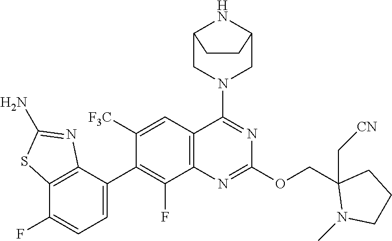 [SMILES: CN1CCCC1(CC#N)COc1nc(N2CC3CCC(C2)N3)c2cc(C(F)(F)F)c(-c3ccc(F)c4sc(N)nc34)c(F)c2n1]

E121

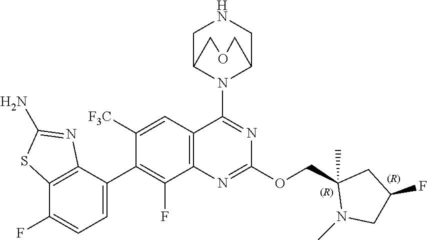

E122

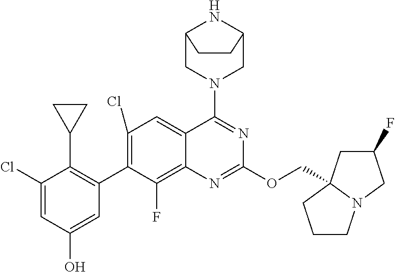 [SMILES: Oc1cc(Cl)c(C2CC2)c(-c2c(Cl)cc3c(N4CC5CCC(C4)N5)nc(OC[C@@]45CCCN4C[C@H](F)C5)nc3c2F)c1]

E123

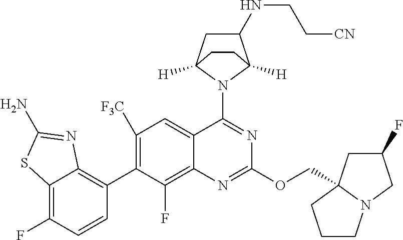 [SMILES: N#CCCNC1C[C@H]2CC[C@@H]1N2c1nc(OC[C@@]23CCCN2C[C@H](F)C3)nc2c(F)c(-c3ccc(F)c4sc(N)nc34)c(C(F)(F)F)cc12]

E124

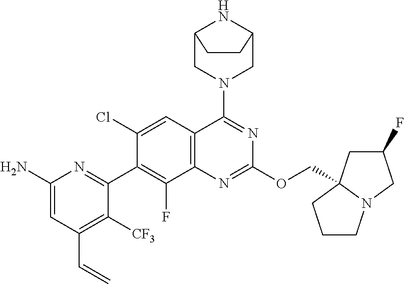 [SMILES: C=Cc1cc(N)nc(-c2c(Cl)cc3c(N4CC5CCC(C4)N5)nc(OC[C@@]45CCCN4C[C@H](F)C5)nc3c2F)c1C(F)(F)F]

E125

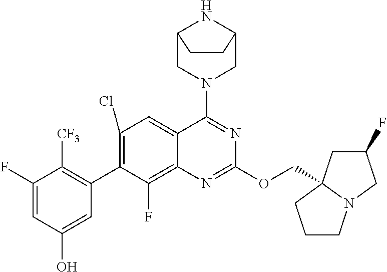 [SMILES: Oc1cc(F)c(C(F)(F)F)c(-c2c(Cl)cc3c(N4CC5CCC(C4)N5)nc(OC[C@@]45CCCN4C[C@H](F)C5)nc3c2F)c1]

E126

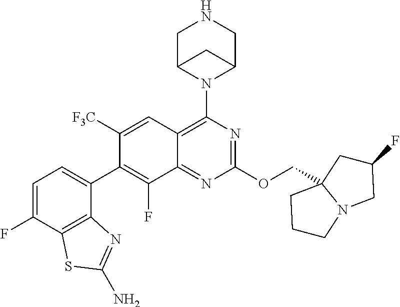

E127

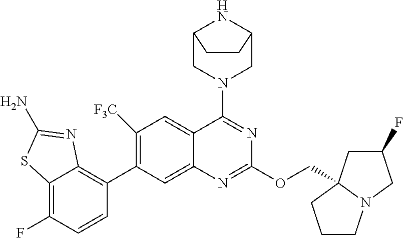 [SMILES: Nc1nc2c(-c3cc4nc(OC[C@@]56CCCN5C[C@H](F)C6)nc(N5CC6CCC(C5)N6)c4cc3C(F)(F)F)ccc(F)c2s1]

E128

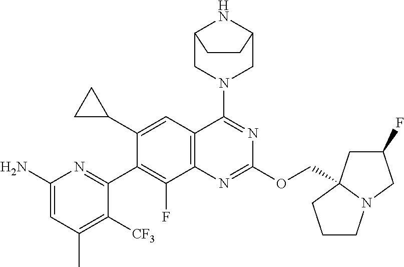 [SMILES: Cc1cc(N)nc(-c2c(C3CC3)cc3c(N4CC5CCC(C4)N5)nc(OC[C@@]45CCCN4C[C@H](F)C5)nc3c2F)c1C(F)(F)F]

E129

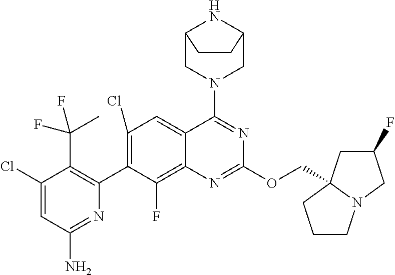 [SMILES: CC(F)(F)c1c(Cl)cc(N)nc1-c1c(Cl)cc2c(N3CC4CCC(C3)N4)nc(OC[C@@]34CCCN3C[C@H](F)C4)nc2c1F]

E130

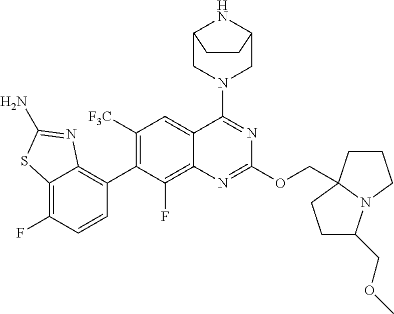 [SMILES: COCC1CCC2(COc3nc(N4CC5CCC(C4)N5)c4cc(C(F)(F)F)c(-c5ccc(F)c6sc(N)nc56)c(F)c4n3)CCCN12]

E131

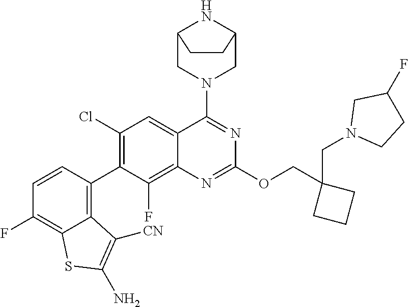 [SMILES: N#Cc1c(N)sc2c(F)ccc(-c3c(Cl)cc4c(N5CC6CCC(C5)N6)nc(OCC5(CN6CCC(F)C6)CCC5)nc4c3F)c12]

E132

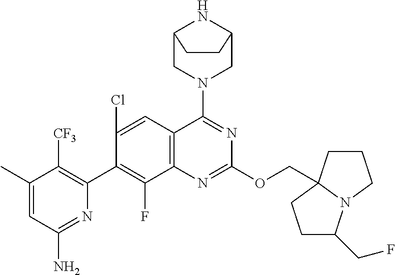 [SMILES: Cc1cc(N)nc(-c2c(Cl)cc3c(N4CC5CCC(C4)N5)nc(OCC45CCCN4C(CF)CC5)nc3c2F)c1C(F)(F)F]

E133

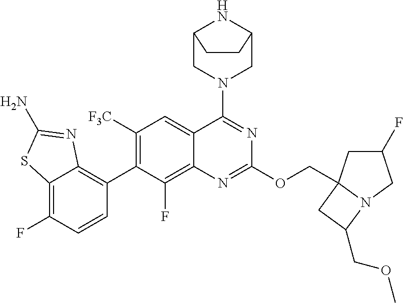 [SMILES: COCC1CC2(COc3nc(N4CC5CCC(C4)N5)c4cc(C(F)(F)F)c(-c5ccc(F)c6sc(N)nc56)c(F)c4n3)CC(F)CN12]

E134

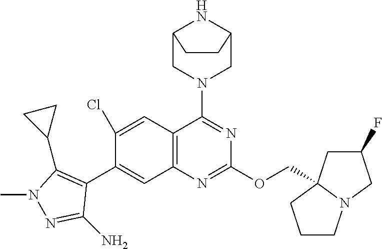 [SMILES: Cn1nc(N)c(-c2cc3nc(OC[C@@]45CCCN4C[C@H](F)C5)nc(N4CC5CCC(C4)N5)c3cc2Cl)c1C1CC1]

E135

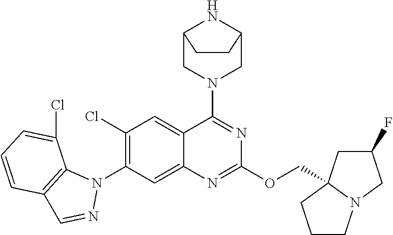 [SMILES: F[C@H]1CN2CCC[C@@]2(COc2nc(N3CC4CCC(C3)N4)c3cc(Cl)c(-n4ncc5cccc(Cl)c54)cc3n2)C1]

E136

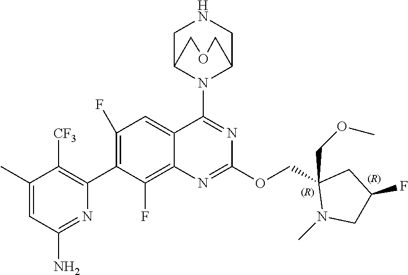

E137

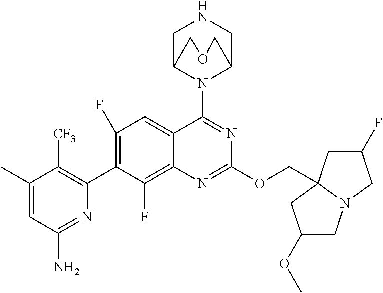 [SMILES: COC1CN2CC(F)CC2(COc2nc(N3C4CNCC3COC4)c3cc(F)c(-c4nc(N)cc(C)c4C(F)(F)F)c(F)c3n2)C1]

E138

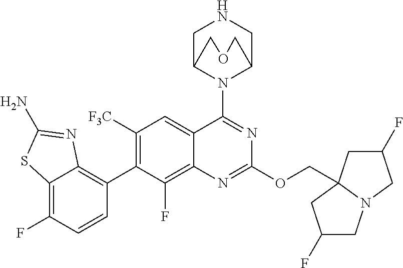 [SMILES: Nc1nc2c(-c3c(C(F)(F)F)cc4c(N5C6CNCC5COC6)nc(OCC56CC(F)CN5CC(F)C6)nc4c3F)ccc(F)c2s1]

E139

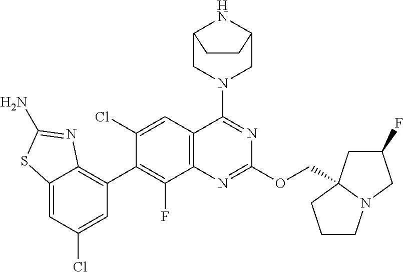 [SMILES: Nc1nc2c(-c3c(Cl)cc4c(N5CC6CCC(C5)N6)nc(OC[C@@]56CCCN5C[C@H](F)C6)nc4c3F)cc(Cl)cc2s1]

E140

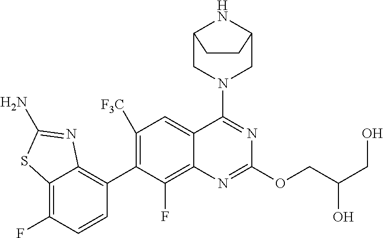 [SMILES: Nc1nc2c(-c3c(C(F)(F)F)cc4c(N5CC6CCC(C5)N6)nc(OCC(O)CO)nc4c3F)ccc(F)c2s1]

E141

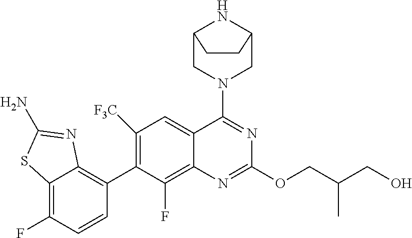 [SMILES: CC(CO)COc1nc(N2CC3CCC(C2)N3)c2cc(C(F)(F)F)c(-c3ccc(F)c4sc(N)nc34)c(F)c2n1]

E142

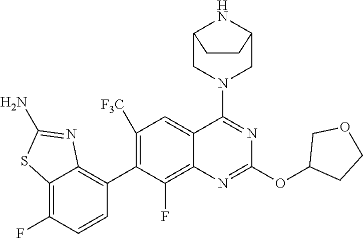 [SMILES: Nc1nc2c(-c3c(C(F)(F)F)cc4c(N5CC6CCC(C5)N6)nc(OC5CCOC5)nc4c3F)ccc(F)c2s1]

E143

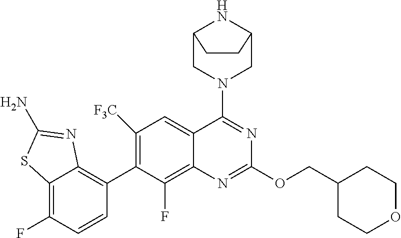 [SMILES: Nc1nc2c(-c3c(C(F)(F)F)cc4c(N5CC6CCC(C5)N6)nc(OCC5CCOCC5)nc4c3F)ccc(F)c2s1]

E144

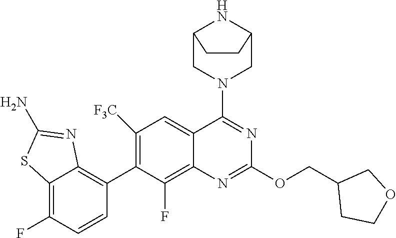 [SMILES: Nc1nc2c(-c3c(C(F)(F)F)cc4c(N5CC6CCC(C5)N6)nc(OCC5CCOC5)nc4c3F)ccc(F)c2s1]

E145

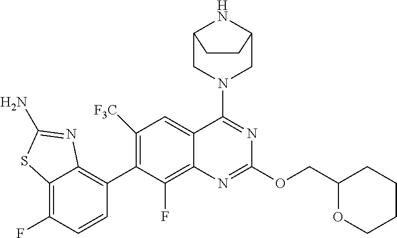 [SMILES: Nc1nc2c(-c3c(C(F)(F)F)cc4c(N5CC6CCC(C5)N6)nc(OCC5CCCCO5)nc4c3F)ccc(F)c2s1]

E146

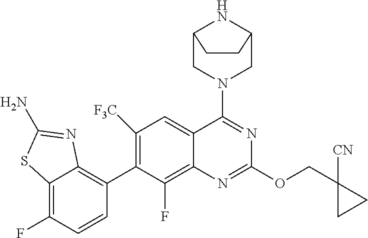 [SMILES: N#CC1(COc2nc(N3CC4CCC(C3)N4)c3cc(C(F)(F)F)c(-c4ccc(F)c5sc(N)nc45)c(F)c3n2)CC1]

E147

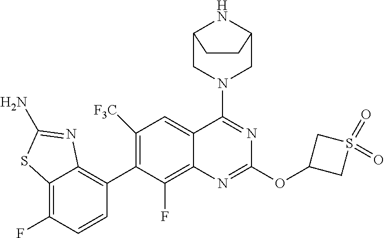 [SMILES: Nc1nc2c(-c3c(C(F)(F)F)cc4c(N5CC6CCC(C5)N6)nc(OC5CS(=O)(=O)C5)nc4c3F)ccc(F)c2s1]

E148

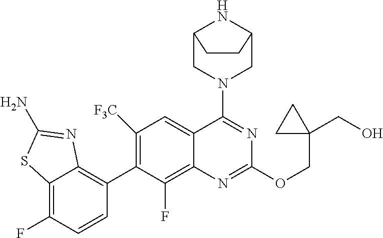 [SMILES: Nc1nc2c(-c3c(C(F)(F)F)cc4c(N5CC6CCC(C5)N6)nc(OCC5(CO)CC5)nc4c3F)ccc(F)c2s1]

E149

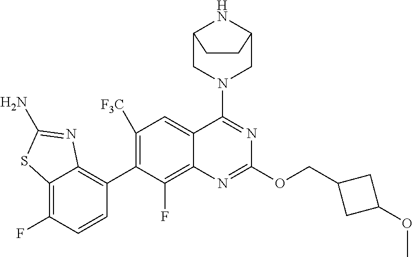 [SMILES: COC1CC(COc2nc(N3CC4CCC(C3)N4)c3cc(C(F)(F)F)c(-c4ccc(F)c5sc(N)nc45)c(F)c3n2)C1]

E150

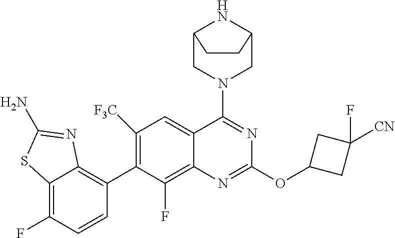 [SMILES: N#CC1(F)CC(Oc2nc(N3CC4CCC(C3)N4)c3cc(C(F)(F)F)c(-c4ccc(F)c5sc(N)nc45)c(F)c3n2)C1]

E151

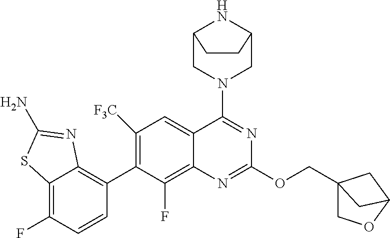 [SMILES: Nc1nc2c(-c3c(C(F)(F)F)cc4c(N5CC6CCC(C5)N6)nc(OCC56COC(C5)C6)nc4c3F)ccc(F)c2s1]

E152

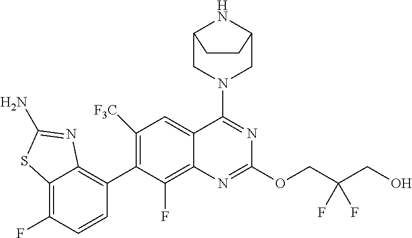 [SMILES: Nc1nc2c(-c3c(C(F)(F)F)cc4c(N5CC6CCC(C5)N6)nc(OCC(F)(F)CO)nc4c3F)ccc(F)c2s1]

E153

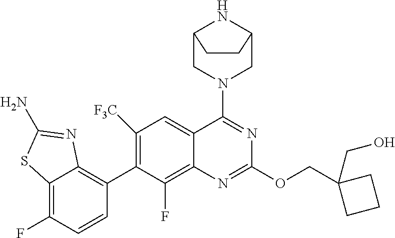 [SMILES: Nc1nc2c(-c3c(C(F)(F)F)cc4c(N5CC6CCC(C5)N6)nc(OCC5(CO)CCC5)nc4c3F)ccc(F)c2s1]

E154

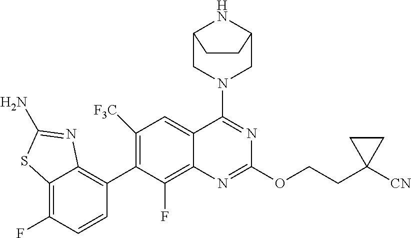 [SMILES: N#CC1(CCOc2nc(N3CC4CCC(C3)N4)c3cc(C(F)(F)F)c(-c4ccc(F)c5sc(N)nc45)c(F)c3n2)CC1]

E155

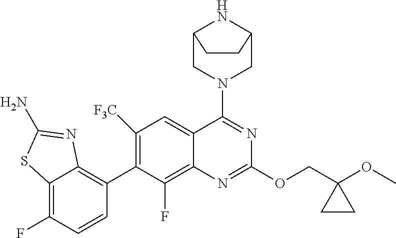 [SMILES: COC1(COc2nc(N3CC4CCC(C3)N4)c3cc(C(F)(F)F)c(-c4ccc(F)c5sc(N)nc45)c(F)c3n2)CC1]

E156

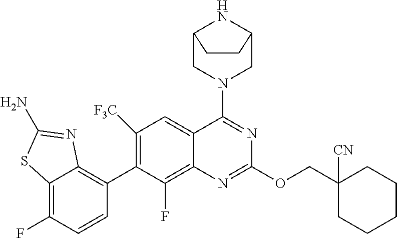 [SMILES: N#CC1(COc2nc(N3CC4CCC(C3)N4)c3cc(C(F)(F)F)c(-c4ccc(F)c5sc(N)nc45)c(F)c3n2)CCCCC1]

E157

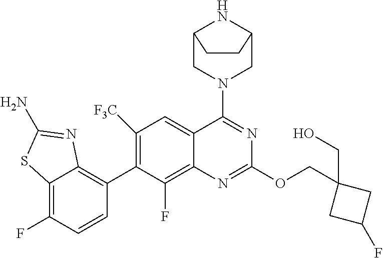 [SMILES: Nc1nc2c(-c3c(C(F)(F)F)cc4c(N5CC6CCC(C5)N6)nc(OCC5(CO)CC(F)C5)nc4c3F)ccc(F)c2s1]

E158

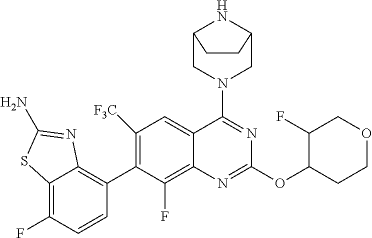 [SMILES: Nc1nc2c(-c3c(C(F)(F)F)cc4c(N5CC6CCC(C5)N6)nc(OC5CCOCC5F)nc4c3F)ccc(F)c2s1]

E159

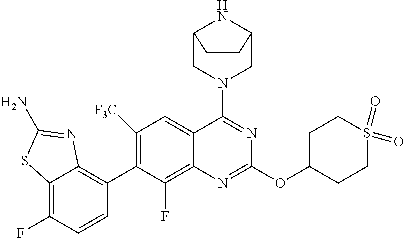 [SMILES: Nc1nc2c(-c3c(C(F)(F)F)cc4c(N5CC6CCC(C5)N6)nc(OC5CCS(=O)(=O)CC5)nc4c3F)ccc(F)c2s1]

E160

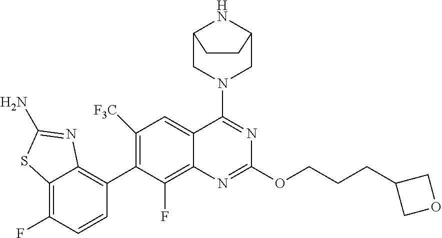 [SMILES: Nc1nc2c(-c3c(C(F)(F)F)cc4c(N5CC6CCC(C5)N6)nc(OCCCC5COC5)nc4c3F)ccc(F)c2s1]

E161

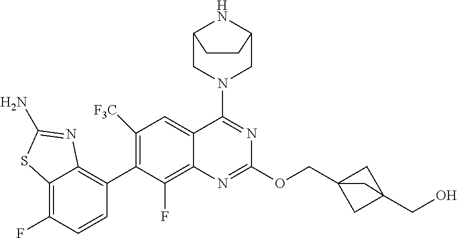 [SMILES: Nc1nc2c(-c3c(C(F)(F)F)cc4c(N5CC6CCC(C5)N6)nc(OCC56CC(CO)(C5)C6)nc4c3F)ccc(F)c2s1]

E162

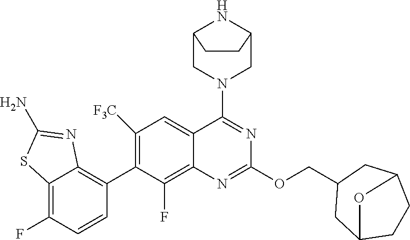 [SMILES: Nc1nc2c(-c3c(C(F)(F)F)cc4c(N5CC6CCC(C5)N6)nc(OCC5CC6CCC(C5)O6)nc4c3F)ccc(F)c2s1]

E163

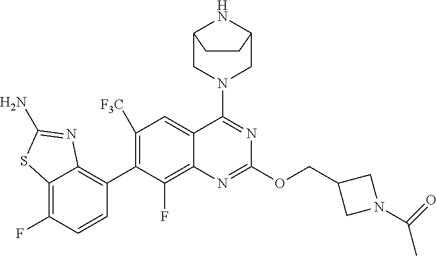 [SMILES: CC(=O)N1CC(COc2nc(N3CC4CCC(C3)N4)c3cc(C(F)(F)F)c(-c4ccc(F)c5sc(N)nc45)c(F)c3n2)C1]

E164

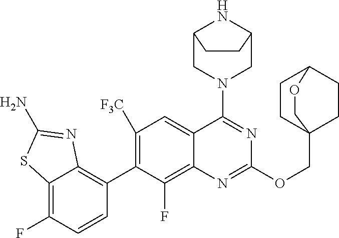 [SMILES: Nc1nc2c(-c3c(C(F)(F)F)cc4c(N5CC6CCC(C5)N6)nc(OCC56CCC(CC5)OC6)nc4c3F)ccc(F)c2s1]

E165

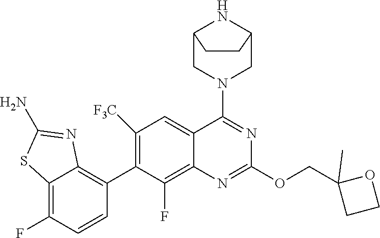 [SMILES: CC1(COc2nc(N3CC4CCC(C3)N4)c3cc(C(F)(F)F)c(-c4ccc(F)c5sc(N)nc45)c(F)c3n2)CCO1]

E166

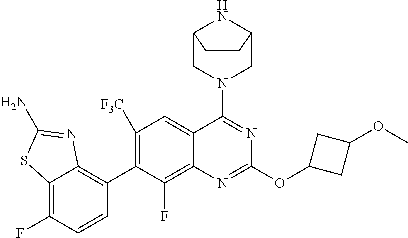 [SMILES: COC1CC(Oc2nc(N3CC4CCC(C3)N4)c3cc(C(F)(F)F)c(-c4ccc(F)c5sc(N)nc45)c(F)c3n2)C1]

E167

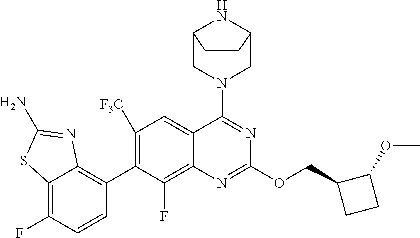 [SMILES: CO[C@@H]1CC[C@H]1COc1nc(N2CC3CCC(C2)N3)c2cc(C(F)(F)F)c(-c3ccc(F)c4sc(N)nc34)c(F)c2n1]

E168

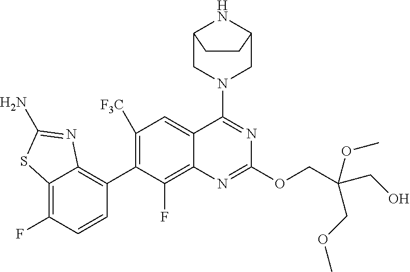 [SMILES: COCC(CO)(COc1nc(N2CC3CCC(C2)N3)c2cc(C(F)(F)F)c(-c3ccc(F)c4sc(N)nc34)c(F)c2n1)OC]

E169

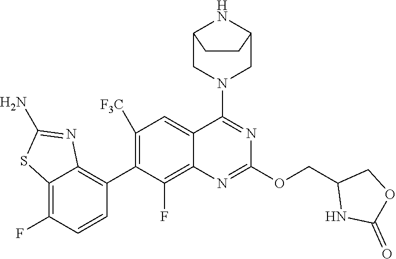 [SMILES: Nc1nc2c(-c3c(C(F)(F)F)cc4c(N5CC6CCC(C5)N6)nc(OCC5COC(=O)N5)nc4c3F)ccc(F)c2s1]

E170

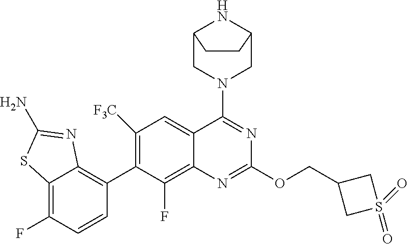 [SMILES: Nc1nc2c(-c3c(C(F)(F)F)cc4c(N5CC6CCC(C5)N6)nc(OCC5CS(=O)(=O)C5)nc4c3F)ccc(F)c2s1]

E171

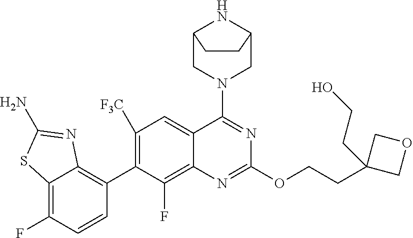 [SMILES: Nc1nc2c(-c3c(C(F)(F)F)cc4c(N5CC6CCC(C5)N6)nc(OCCC5(CCO)COC5)nc4c3F)ccc(F)c2s1]

E172

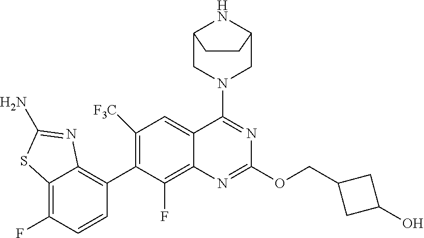 [SMILES: Nc1nc2c(-c3c(C(F)(F)F)cc4c(N5CC6CCC(C5)N6)nc(OCC5CC(O)C5)nc4c3F)ccc(F)c2s1]

E173

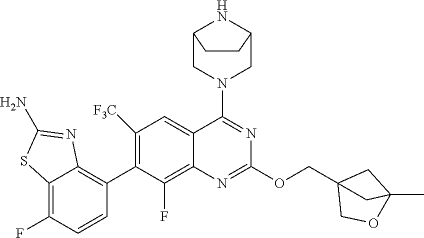 [SMILES: CC12CC(COc3nc(N4CC5CCC(C4)N5)c4cc(C(F)(F)F)c(-c5ccc(F)c6sc(N)nc56)c(F)c4n3)(CO1)C2]

E174

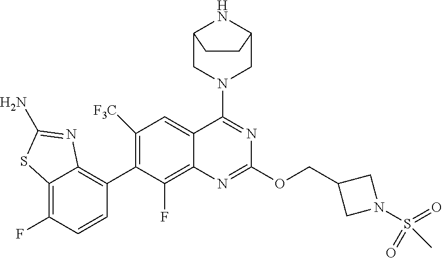 [SMILES: CS(=O)(=O)N1CC(COc2nc(N3CC4CCC(C3)N4)c3cc(C(F)(F)F)c(-c4ccc(F)c5sc(N)nc45)c(F)c3n2)C1]

E175

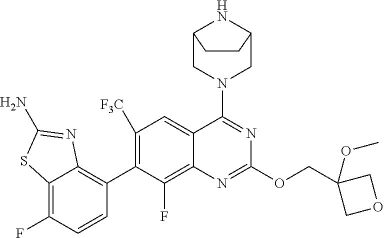 [SMILES: COC1(COc2nc(N3CC4CCC(C3)N4)c3cc(C(F)(F)F)c(-c4ccc(F)c5sc(N)nc45)c(F)c3n2)COC1]

E176

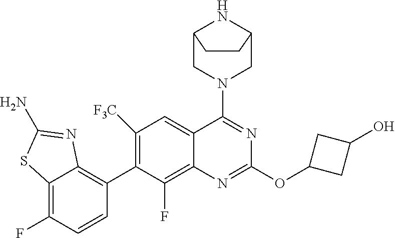 [SMILES: Nc1nc2c(-c3c(C(F)(F)F)cc4c(N5CC6CCC(C5)N6)nc(OC5CC(O)C5)nc4c3F)ccc(F)c2s1]

E177

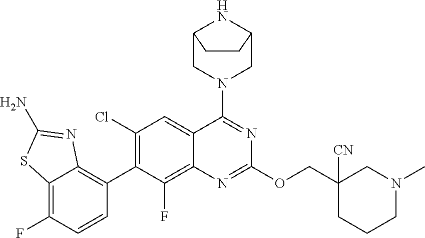 [SMILES: CN1CCCC(C#N)(COc2nc(N3CC4CCC(C3)N4)c3cc(Cl)c(-c4ccc(F)c5sc(N)nc45)c(F)c3n2)C1]

E178

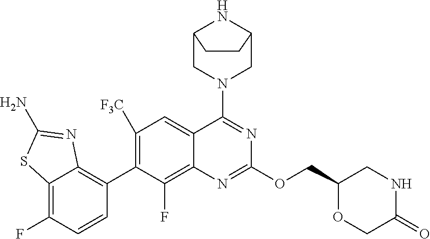 [SMILES: Nc1nc2c(-c3c(C(F)(F)F)cc4c(N5CC6CCC(C5)N6)nc(OC[C@H]5CNC(=O)CO5)nc4c3F)ccc(F)c2s1]

E179

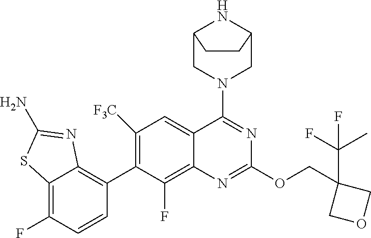 [SMILES: CC(F)(F)C1(COc2nc(N3CC4CCC(C3)N4)c3cc(C(F)(F)F)c(-c4ccc(F)c5sc(N)nc45)c(F)c3n2)COC1]

E180

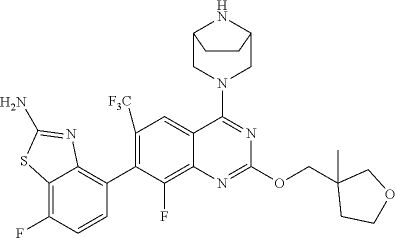 [SMILES: CC1(COc2nc(N3CC4CCC(C3)N4)c3cc(C(F)(F)F)c(-c4ccc(F)c5sc(N)nc45)c(F)c3n2)CCOC1]

E181

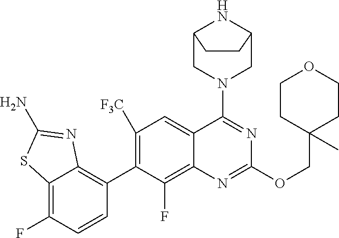 [SMILES: CC1(COc2nc(N3CC4CCC(C3)N4)c3cc(C(F)(F)F)c(-c4ccc(F)c5sc(N)nc45)c(F)c3n2)CCOCC1]

E182

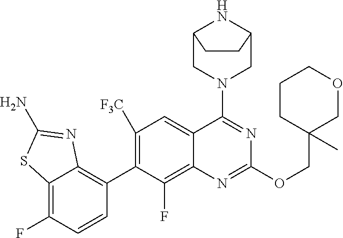 [SMILES: CC1(COc2nc(N3CC4CCC(C3)N4)c3cc(C(F)(F)F)c(-c4ccc(F)c5sc(N)nc45)c(F)c3n2)CCCOC1]

E183

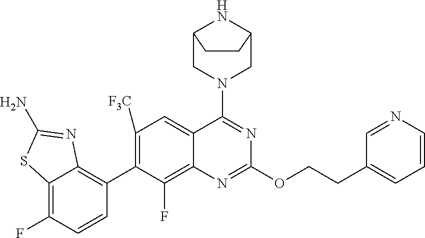 [SMILES: Nc1nc2c(-c3c(C(F)(F)F)cc4c(N5CC6CCC(C5)N6)nc(OCCc5cccnc5)nc4c3F)ccc(F)c2s1]

E184

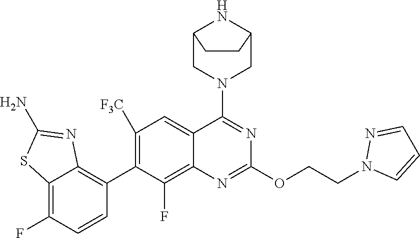 [SMILES: Nc1nc2c(-c3c(C(F)(F)F)cc4c(N5CC6CCC(C5)N6)nc(OCCn5cccn5)nc4c3F)ccc(F)c2s1]

E185

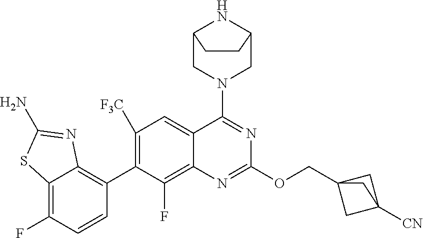 [SMILES: N#CC12CC(COc3nc(N4CC5CCC(C4)N5)c4cc(C(F)(F)F)c(-c5ccc(F)c6sc(N)nc56)c(F)c4n3)(C1)C2]

E186

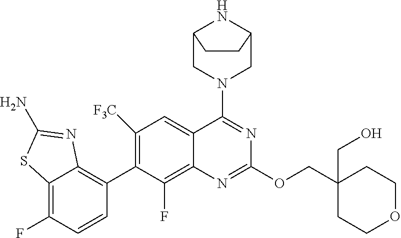 [SMILES: Nc1nc2c(-c3c(C(F)(F)F)cc4c(N5CC6CCC(C5)N6)nc(OCC5(CO)CCOCC5)nc4c3F)ccc(F)c2s1]

E187

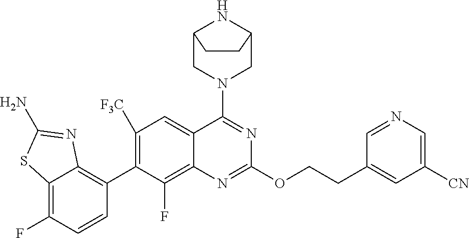 [SMILES: N#Cc1cncc(CCOc2nc(N3CC4CCC(C3)N4)c3cc(C(F)(F)F)c(-c4ccc(F)c5sc(N)nc45)c(F)c3n2)c1]

E188

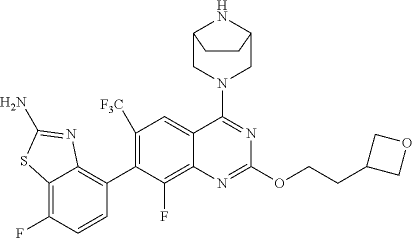 [SMILES: Nc1nc2c(-c3c(C(F)(F)F)cc4c(N5CC6CCC(C5)N6)nc(OCCC5COC5)nc4c3F)ccc(F)c2s1]

E189

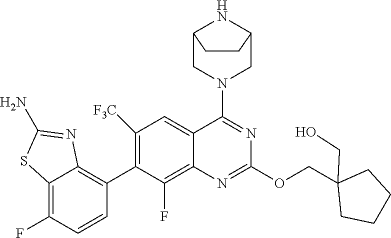 [SMILES: Nc1nc2c(-c3c(C(F)(F)F)cc4c(N5CC6CCC(C5)N6)nc(OCC5(CO)CCCC5)nc4c3F)ccc(F)c2s1]

E190

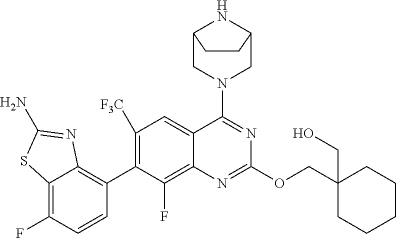 [SMILES: Nc1nc2c(-c3c(C(F)(F)F)cc4c(N5CC6CCC(C5)N6)nc(OCC5(CO)CCCCC5)nc4c3F)ccc(F)c2s1]

E191

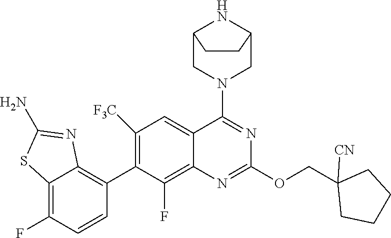 [SMILES: N#CC1(COc2nc(N3CC4CCC(C3)N4)c3cc(C(F)(F)F)c(-c4ccc(F)c5sc(N)nc45)c(F)c3n2)CCCC1]

E192

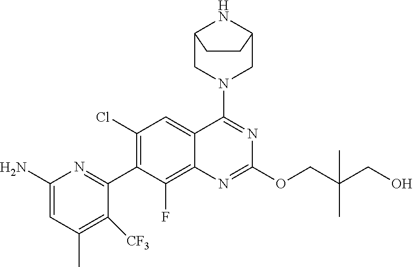 [SMILES: Cc1cc(N)nc(-c2c(Cl)cc3c(N4CC5CCC(C4)N5)nc(OCC(C)(C)CO)nc3c2F)c1C(F)(F)F]

E193

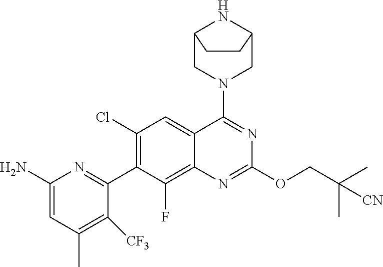 [SMILES: Cc1cc(N)nc(-c2c(Cl)cc3c(N4CC5CCC(C4)N5)nc(OCC(C)(C)C#N)nc3c2F)c1C(F)(F)F]

E194

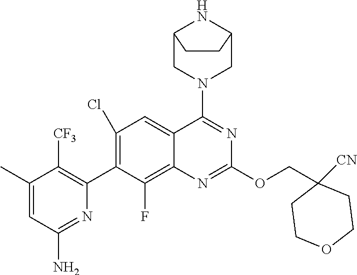 [SMILES: Cc1cc(N)nc(-c2c(Cl)cc3c(N4CC5CCC(C4)N5)nc(OCC4(C#N)CCOCC4)nc3c2F)c1C(F)(F)F]

E195

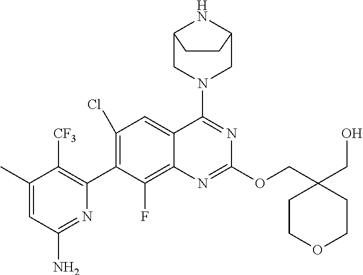 [SMILES: Cc1cc(N)nc(-c2c(Cl)cc3c(N4CC5CCC(C4)N5)nc(OCC4(CO)CCOCC4)nc3c2F)c1C(F)(F)F]

E196

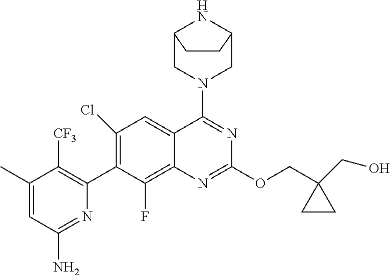 [SMILES: Cc1cc(N)nc(-c2c(Cl)cc3c(N4CC5CCC(C4)N5)nc(OCC4(CO)CC4)nc3c2F)c1C(F)(F)F]

E197

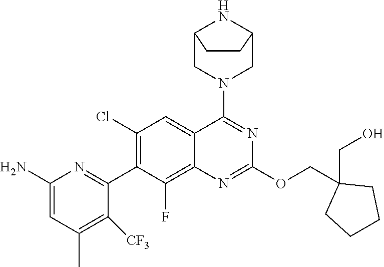 [SMILES: Cc1cc(N)nc(-c2c(Cl)cc3c(N4CC5CCC(C4)N5)nc(OCC4(CO)CCCC4)nc3c2F)c1C(F)(F)F]

E198

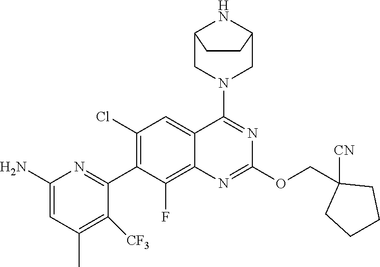 [SMILES: Cc1cc(N)nc(-c2c(Cl)cc3c(N4CC5CCC(C4)N5)nc(OCC4(C#N)CCCC4)nc3c2F)c1C(F)(F)F]

E199

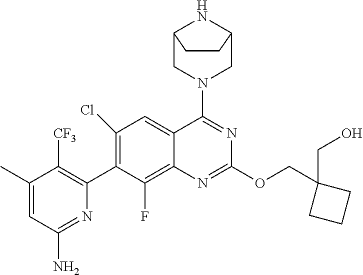 [SMILES: Cc1cc(N)nc(-c2c(Cl)cc3c(N4CC5CCC(C4)N5)nc(OCC4(CO)CCC4)nc3c2F)c1C(F)(F)F]

E200

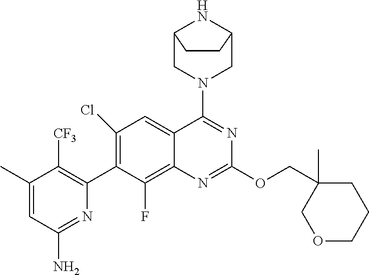 [SMILES: Cc1cc(N)nc(-c2c(Cl)cc3c(N4CC5CCC(C4)N5)nc(OCC4(C)CCCOC4)nc3c2F)c1C(F)(F)F]

E201

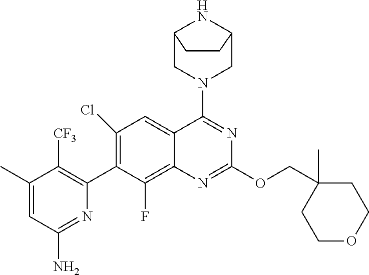 [SMILES: Cc1cc(N)nc(-c2c(Cl)cc3c(N4CC5CCC(C4)N5)nc(OCC4(C)CCOCC4)nc3c2F)c1C(F)(F)F]

E202

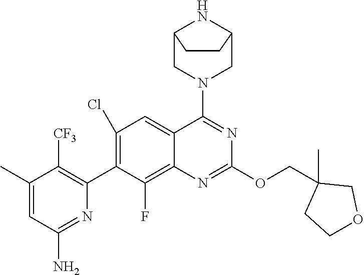 [SMILES: Cc1cc(N)nc(-c2c(Cl)cc3c(N4CC5CCC(C4)N5)nc(OCC4(C)CCOC4)nc3c2F)c1C(F)(F)F]

E203

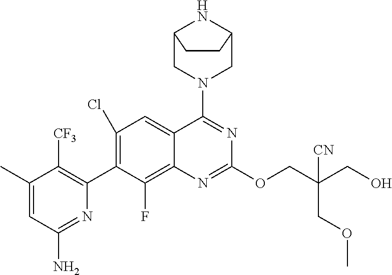 [SMILES: COCC(C#N)(CO)COc1nc(N2CC3CCC(C2)N3)c2cc(Cl)c(-c3nc(N)cc(C)c3C(F)(F)F)c(F)c2n1]

E204

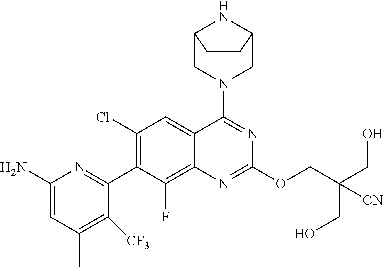 [SMILES: Cc1cc(N)nc(-c2c(Cl)cc3c(N4CC5CCC(C4)N5)nc(OCC(C#N)(CO)CO)nc3c2F)c1C(F)(F)F]

E205

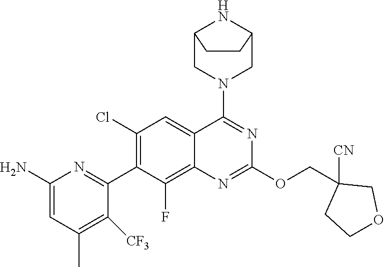 [SMILES: Cc1cc(N)nc(-c2c(Cl)cc3c(N4CC5CCC(C4)N5)nc(OCC4(C#N)CCOC4)nc3c2F)c1C(F)(F)F]

E206

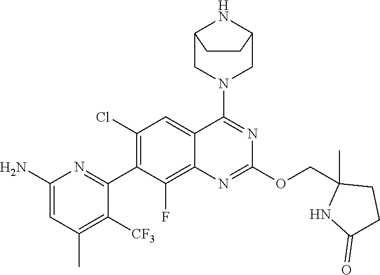 [SMILES: Cc1cc(N)nc(-c2c(Cl)cc3c(N4CC5CCC(C4)N5)nc(OCC4(C)CCC(=O)N4)nc3c2F)c1C(F)(F)F]

E207

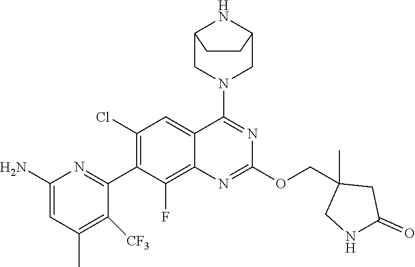 [SMILES: Cc1cc(N)nc(-c2c(Cl)cc3c(N4CC5CCC(C4)N5)nc(OCC4(C)CNC(=O)C4)nc3c2F)c1C(F)(F)F]

E208

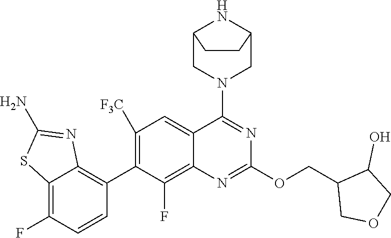 [SMILES: Nc1nc2c(-c3c(C(F)(F)F)cc4c(N5CC6CCC(C5)N6)nc(OCC5COCC5O)nc4c3F)ccc(F)c2s1]

E209

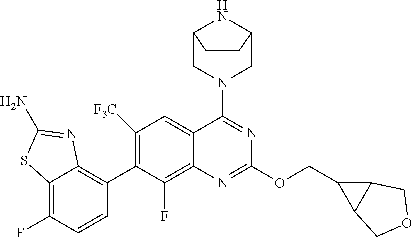 [SMILES: Nc1nc2c(-c3c(C(F)(F)F)cc4c(N5CC6CCC(C5)N6)nc(OCC5C6COCC65)nc4c3F)ccc(F)c2s1]

E210

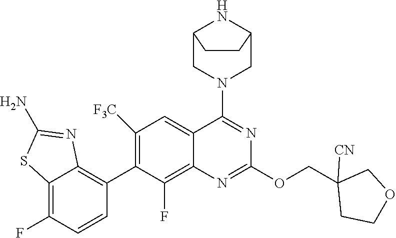 [SMILES: N#CC1(COc2nc(N3CC4CCC(C3)N4)c3cc(C(F)(F)F)c(-c4ccc(F)c5sc(N)nc45)c(F)c3n2)CCOC1]

E211

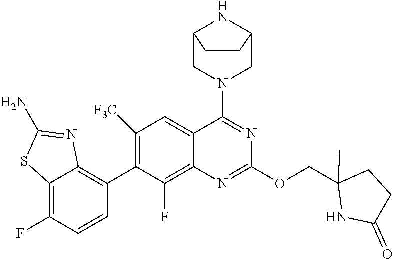 [SMILES: CC1(COc2nc(N3CC4CCC(C3)N4)c3cc(C(F)(F)F)c(-c4ccc(F)c5sc(N)nc45)c(F)c3n2)CCC(=O)N1]

E212

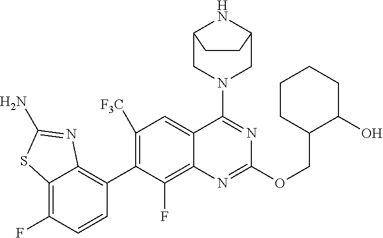 [SMILES: Nc1nc2c(-c3c(C(F)(F)F)cc4c(N5CC6CCC(C5)N6)nc(OCC5CCCCC5O)nc4c3F)ccc(F)c2s1]

E213

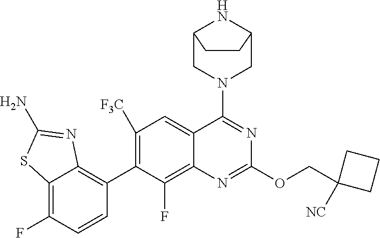 [SMILES: N#CC1(COc2nc(N3CC4CCC(C3)N4)c3cc(C(F)(F)F)c(-c4ccc(F)c5sc(N)nc45)c(F)c3n2)CCC1]

E214

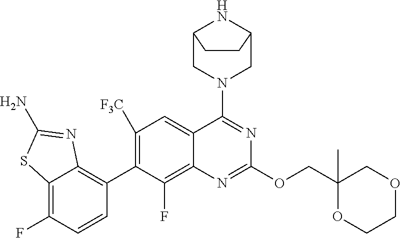 [SMILES: CC1(COc2nc(N3CC4CCC(C3)N4)c3cc(C(F)(F)F)c(-c4ccc(F)c5sc(N)nc45)c(F)c3n2)COCCO1]

E215

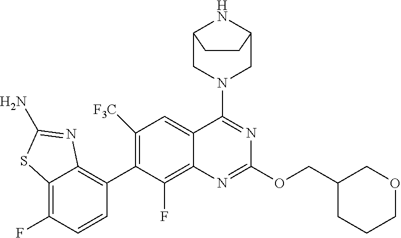 [SMILES: Nc1nc2c(-c3c(C(F)(F)F)cc4c(N5CC6CCC(C5)N6)nc(OCC5CCCOC5)nc4c3F)ccc(F)c2s1]

E216

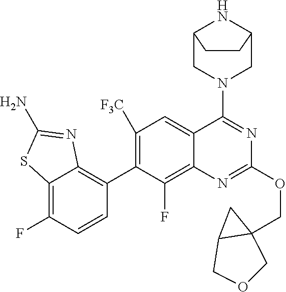 [SMILES: Nc1nc2c(-c3c(C(F)(F)F)cc4c(N5CC6CCC(C5)N6)nc(OCC56COCC5C6)nc4c3F)ccc(F)c2s1]

E217

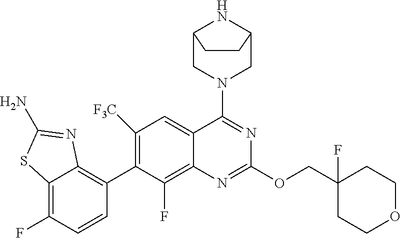 [SMILES: Nc1nc2c(-c3c(C(F)(F)F)cc4c(N5CC6CCC(C5)N6)nc(OCC5(F)CCOCC5)nc4c3F)ccc(F)c2s1]

E218

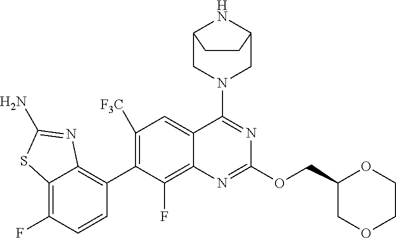 [SMILES: Nc1nc2c(-c3c(C(F)(F)F)cc4c(N5CC6CCC(C5)N6)nc(OC[C@@H]5COCCO5)nc4c3F)ccc(F)c2s1]

E219

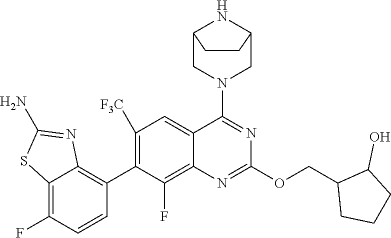 [SMILES: Nc1nc2c(-c3c(C(F)(F)F)cc4c(N5CC6CCC(C5)N6)nc(OCC5CCCC5O)nc4c3F)ccc(F)c2s1]

E220

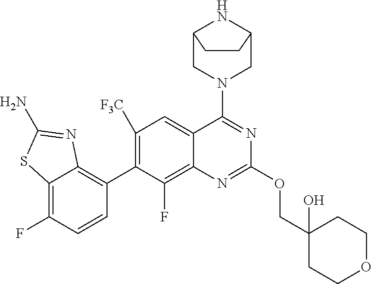 [SMILES: Nc1nc2c(-c3c(C(F)(F)F)cc4c(N5CC6CCC(C5)N6)nc(OCC5(O)CCOCC5)nc4c3F)ccc(F)c2s1]

E221

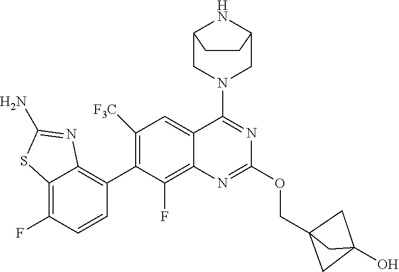 [SMILES: Nc1nc2c(-c3c(C(F)(F)F)cc4c(N5CC6CCC(C5)N6)nc(OCC56CC(O)(C5)C6)nc4c3F)ccc(F)c2s1]

E222

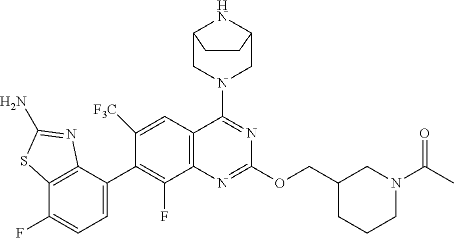 [SMILES: CC(=O)N1CCCC(COc2nc(N3CC4CCC(C3)N4)c3cc(C(F)(F)F)c(-c4ccc(F)c5sc(N)nc45)c(F)c3n2)C1]

E223

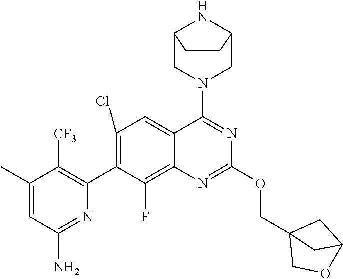 [SMILES: Cc1cc(N)nc(-c2c(Cl)cc3c(N4CC5CCC(C4)N5)nc(OCC45COC(C4)C5)nc3c2F)c1C(F)(F)F]

E224

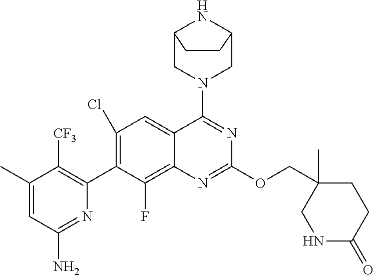 [SMILES: Cc1cc(N)nc(-c2c(Cl)cc3c(N4CC5CCC(C4)N5)nc(OCC4(C)CCC(=O)NC4)nc3c2F)c1C(F)(F)F]

E225

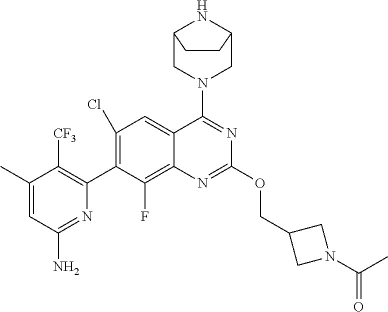 [SMILES: CC(=O)N1CC(COc2nc(N3CC4CCC(C3)N4)c3cc(Cl)c(-c4nc(N)cc(C)c4C(F)(F)F)c(F)c3n2)C1]

E226

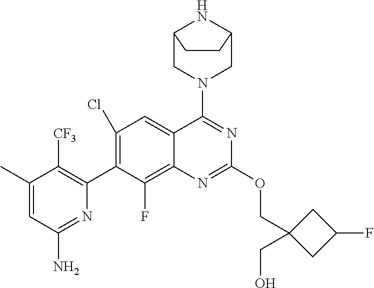 [SMILES: Cc1cc(N)nc(-c2c(Cl)cc3c(N4CC5CCC(C4)N5)nc(OCC4(CO)CC(F)C4)nc3c2F)c1C(F)(F)F]

E227

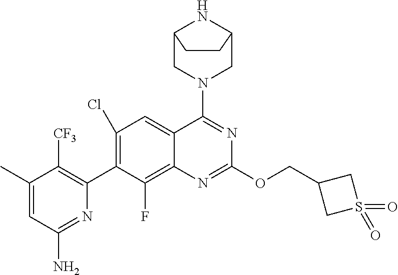 [SMILES: Cc1cc(N)nc(-c2c(Cl)cc3c(N4CC5CCC(C4)N5)nc(OCC4CS(=O)(=O)C4)nc3c2F)c1C(F)(F)F]

E228

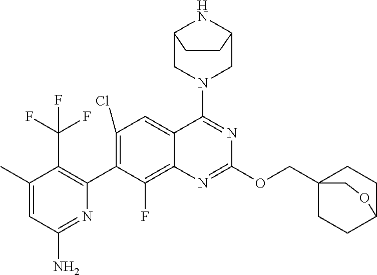 [SMILES: Cc1cc(N)nc(-c2c(Cl)cc3c(N4CC5CCC(C4)N5)nc(OCC45CCC(CC4)OC5)nc3c2F)c1C(F)(F)F]

E229

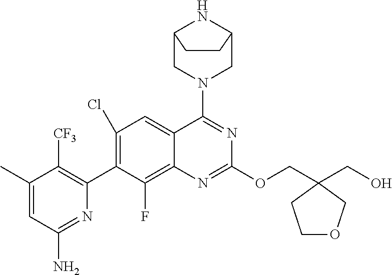 [SMILES: Cc1cc(N)nc(-c2c(Cl)cc3c(N4CC5CCC(C4)N5)nc(OCC4(CO)CCOC4)nc3c2F)c1C(F)(F)F]

E230

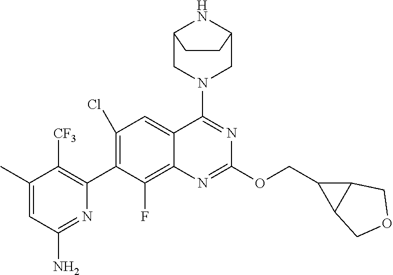 [SMILES: Cc1cc(N)nc(-c2c(Cl)cc3c(N4CC5CCC(C4)N5)nc(OCC4C5COCC54)nc3c2F)c1C(F)(F)F]

E231

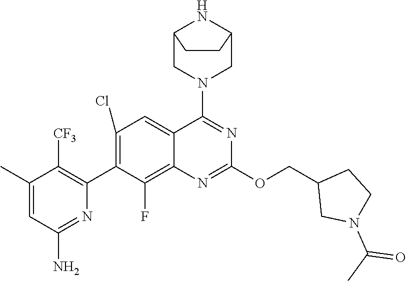 [SMILES: CC(=O)N1CCC(COc2nc(N3CC4CCC(C3)N4)c3cc(Cl)c(-c4nc(N)cc(C)c4C(F)(F)F)c(F)c3n2)C1]

E232

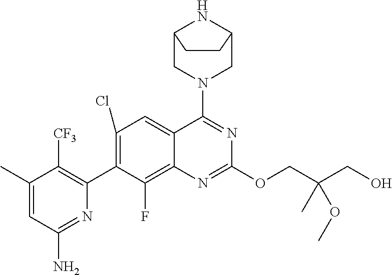 [SMILES: COC(C)(CO)COc1nc(N2CC3CCC(C2)N3)c2cc(Cl)c(-c3nc(N)cc(C)c3C(F)(F)F)c(F)c2n1]

E233

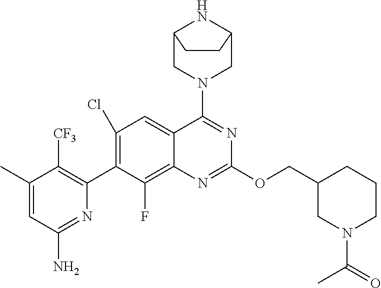 [SMILES: CC(=O)N1CCCC(COc2nc(N3CC4CCC(C3)N4)c3cc(Cl)c(-c4nc(N)cc(C)c4C(F)(F)F)c(F)c3n2)C1]

E234

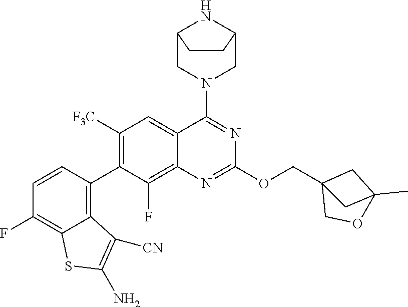 [SMILES: CC12CC(COc3nc(N4CC5CCC(C4)N5)c4cc(C(F)(F)F)c(-c5ccc(F)c6sc(N)c(C#N)c56)c(F)c4n3)(CO1)C2]

E235

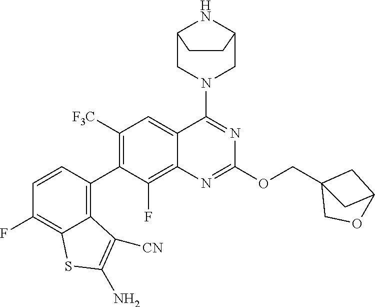 [SMILES: N#Cc1c(N)sc2c(F)ccc(-c3c(C(F)(F)F)cc4c(N5CC6CCC(C5)N6)nc(OCC56COC(C5)C6)nc4c3F)c12]

E236

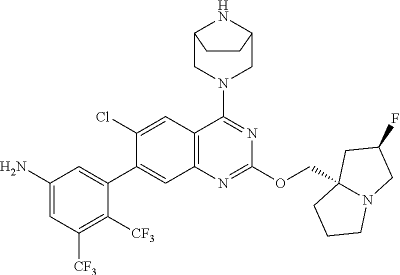 [SMILES: Nc1cc(-c2cc3nc(OC[C@@]45CCCN4C[C@H](F)C5)nc(N4CC5CCC(C4)N5)c3cc2Cl)c(C(F)(F)F)c(C(F)(F)F)c1]

E237

Note: The E numbers of the compounds correspond to the example numbers where the compounds are the final products, respectively.

In some embodiments, the compound provided herein is 4-(4-(3-oxa-7,9-diazabicyclo[3.3.1]nonan-9-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine, 7-fluoro-4-(8-fluoro-2-(((2R,7aS)-2-fluorotetrahydro-1H-pyrrolizin-7a(5H)-yl)methoxy)-4-((1S,4R)-2-((2-methoxyethyl)amino)-7-azabicyclo[2.2.1]heptan-7-yl)-6-(trifluoromethyl)quinazolin-7-yl)benzo[d]thiazol-2-amine, 4-(4-(3,6-diazabicyclo[3.1.1]heptan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine, 4-(4-(3,8-diazabicyclo[3.2.1]octan-8-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine,

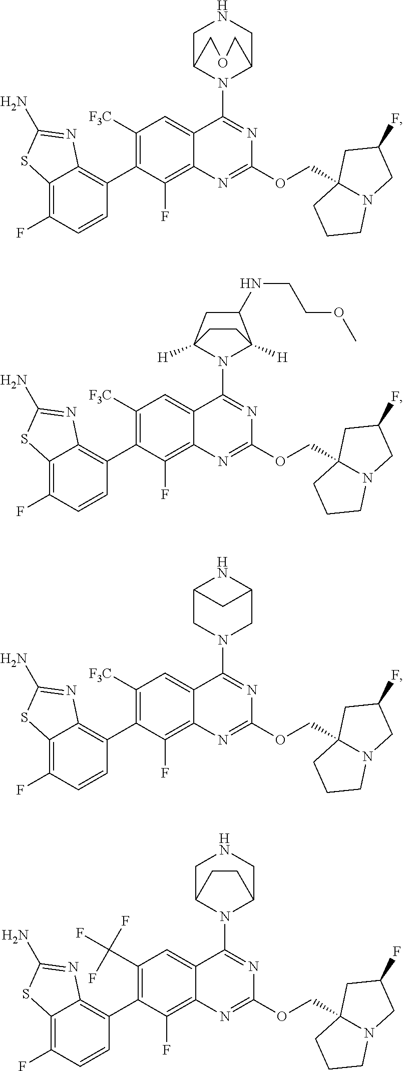 [SMILES: Nc1nc2c(-c3c(C(F)(F)F)cc4c(N5C6CNCC5COC6)nc(OC[C@@]56CCCN5C[C@H](F)C6)nc4c3F)ccc(F)c2s1 ; COCCNC1C[C@H]2CC[C@@H]1N2c1nc(OC[C@@]23CCCN2C[C@H](F)C3)nc2c(F)c(-c3ccc(F)c4sc(N)nc34)c(C(F)(F)F)cc12 ; Nc1nc2c(-c3c(C(F)(F)F)cc4c(N5CC6CC(C5)N6)nc(OC[C@@]56CCCN5C[C@H](F)C6)nc4c3F)ccc(F)c2s1 ; Nc1nc2c(-c3c(C(F)(F)F)cc4c(N5C6CCC5CNC6)nc(OC[C@@]56CCCN5C[C@H](F)C6)nc4c3F)ccc(F)c2s1]

or a pharmaceutically acceptable salt, tautomer, stereoisomer, enantiomer, isotopologue, or prodrug thereof.

In some embodiments, the compound provided herein is 4-(4-(3,6-diazabicyclo[3.1.1]heptan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine,

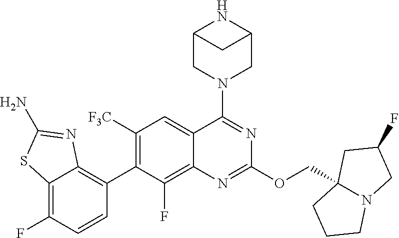 [SMILES: Nc1nc2c(-c3c(C(F)(F)F)cc4c(N5CC6CC(C5)N6)nc(OC[C@@]56CCCN5C[C@H](F)C6)nc4c3F)ccc(F)c2s1]

or a pharmaceutically acceptable salt, tautomer, stereoisomer, enantiomer, isotopologue, or prodrug thereof.

In some embodiments, the compound provided herein is 7-(2-amino-7-fluorobenzo[d]thiazol-4-yl)-4-(3,8-diazabicyclo[3.2.1]octan-3-yl)-6-chloro-2-(3-(dimethylamino)propyl)-8-fluorophthalazin-1(2H)-one,

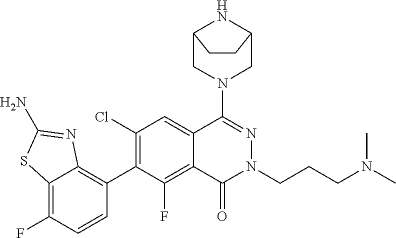 [SMILES: CN(C)CCCn1nc(N2CC3CCC(C2)N3)c2cc(Cl)c(-c3ccc(F)c4sc(N)nc34)c(F)c2c1=O]

or a pharmaceutically acceptable salt, tautomer, stereoisomer, enantiomer, isotopologue, or prodrug thereof.

Aspect 39a: Provided herein are bridged compounds having the following formula (IX):

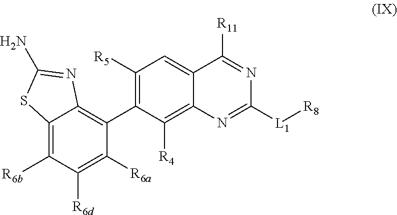

and pharmaceutically acceptable salts, tautomers, isotopologues, stereoisomers and prodrugs thereof, wherein R₄ is F, Cl or methyl;
R₅ is —CF₃, —Cl, —OCF₃, —SCF₃, —CF₂CH₃, —CH₂CHF₂, —CN, —CHF₂, ethyl, isopropyl,

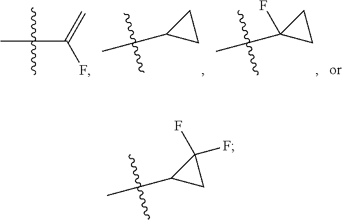

R₆ₐ is H, F, Cl or methyl;
R_{6b} is H, F, Cl or methyl;
R_{6d} is H, F, Cl or methyl;
-L₁-R₈ is

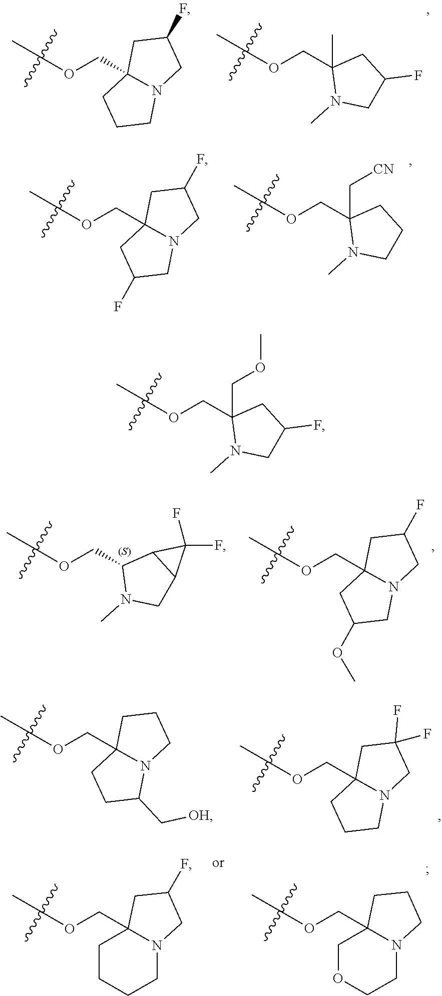

R₁₁ is

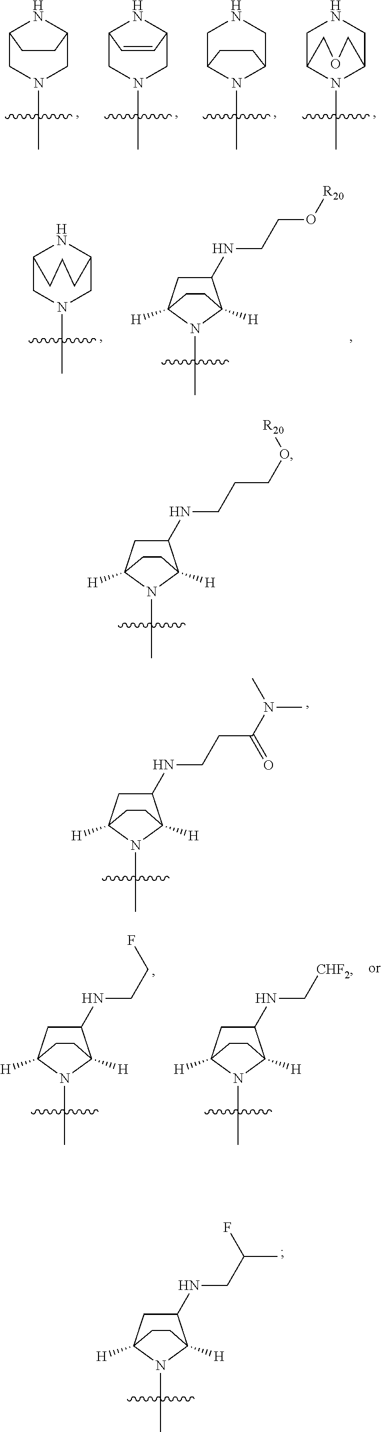

and
R₂₀ is methyl, —CHF₂, CF₃, —CH₂F, or —CD₃.

Aspect 39b: Provided herein are bridged compounds having the formula (IX) and pharmaceutically acceptable salts, tautomers, isotopologues, stereoisomers and prodrugs thereof,
wherein R₄ is F, Cl or methyl;
R₅ is —CF₃;
R₆ₐ is H, F, Cl or methyl;
R_{6b} is F, Cl or methyl;
R_{6d} is H, F or Cl;
-L₁-R₈ is

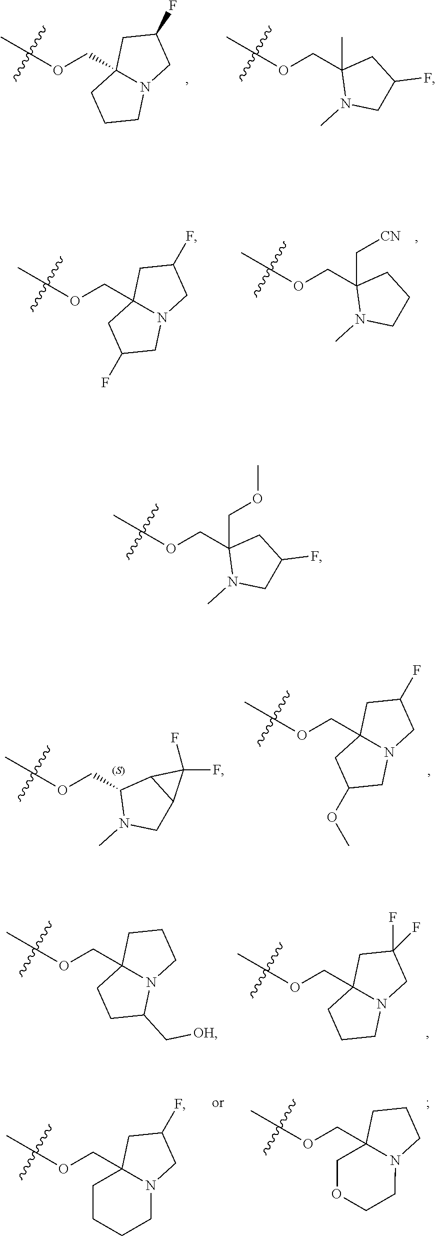

R₁₁ is

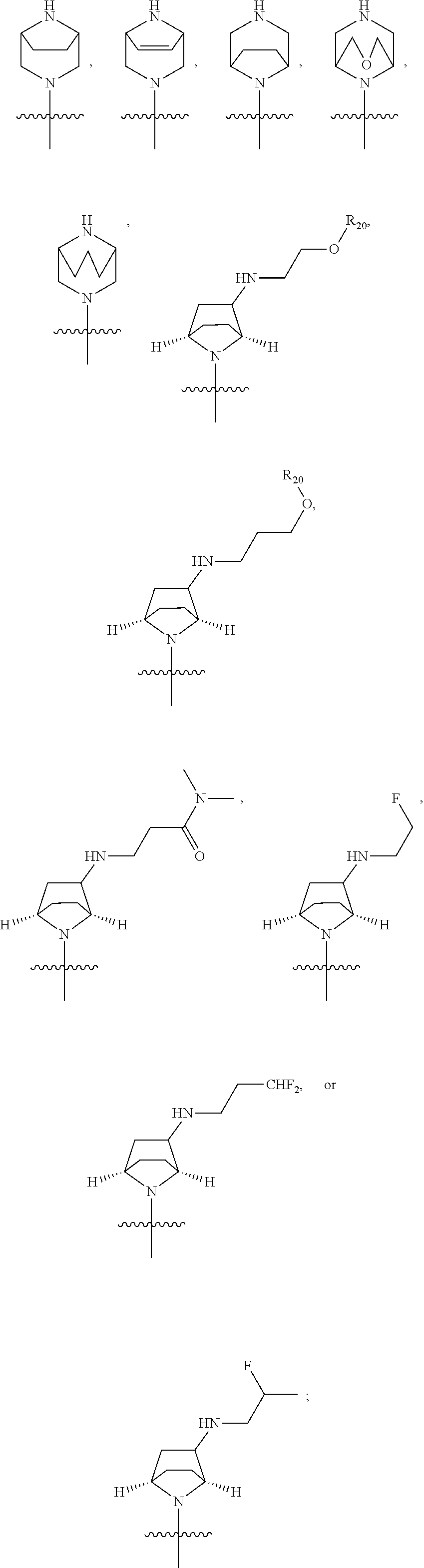

and
R₂₀ is methyl, —CHF₂, CF₃, —CH₂F, or —CD₃.

Aspect 39c: Provided herein are bridged compounds having the formula (IX) and pharmaceutically acceptable salts, tautomers, isotopologues, stereoisomers and prodrugs thereof,
wherein R₄ is F, Cl or methyl;
R₅ is —CF₃, —Cl, —OCF₃, —SCF₃, —CF₂CH₃, —CH₂CHF₂, —CN, —CHF₂, ethyl, isopropyl,

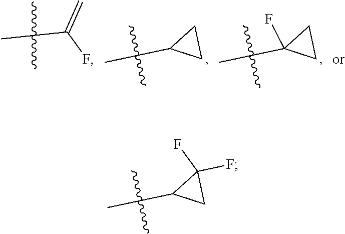

R₆ₐ is H, F, Cl or methyl;
R_{6b} is F, Cl or methyl;
R_{6d} is H, F or Cl;
-L₁-R₈ is

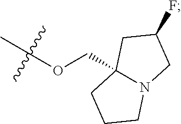 [SMILES: *OC[C@@]12CCCN1C[C@H](F)C2]

R₁₁ is

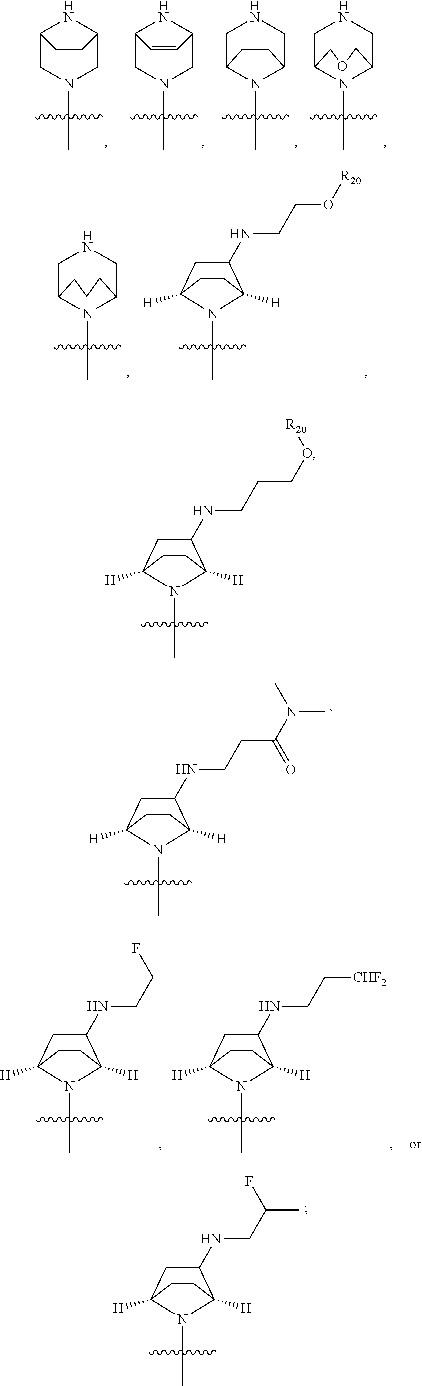

and
R₂₀ is methyl, —CHF₂, CF₃, —CH₂F, or —CD₃.

Aspect 39d: Provided herein are bridged compounds having the formula (IX) and pharmaceutically acceptable salts, tautomers, isotopologues, stereoisomers and prodrugs thereof,
wherein R₄ is F, Cl or methyl;
R₅ is —CF₃, —Cl, —OCF₃, —SCF₃, —CF₂CH₃, —CH₂CHF₂, —CN, —CHF₂, ethyl, isopropyl

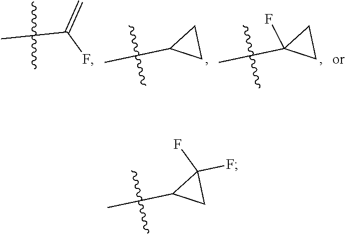

R₆ₐ is H, F, Cl or methyl;
R_{6b} is F, Cl or methyl;
R_{6d} is H;
-L₁-R₈ is

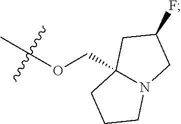 [SMILES: *OC[C@@]12CCCN1C[C@H](F)C2]

R₁₁ is

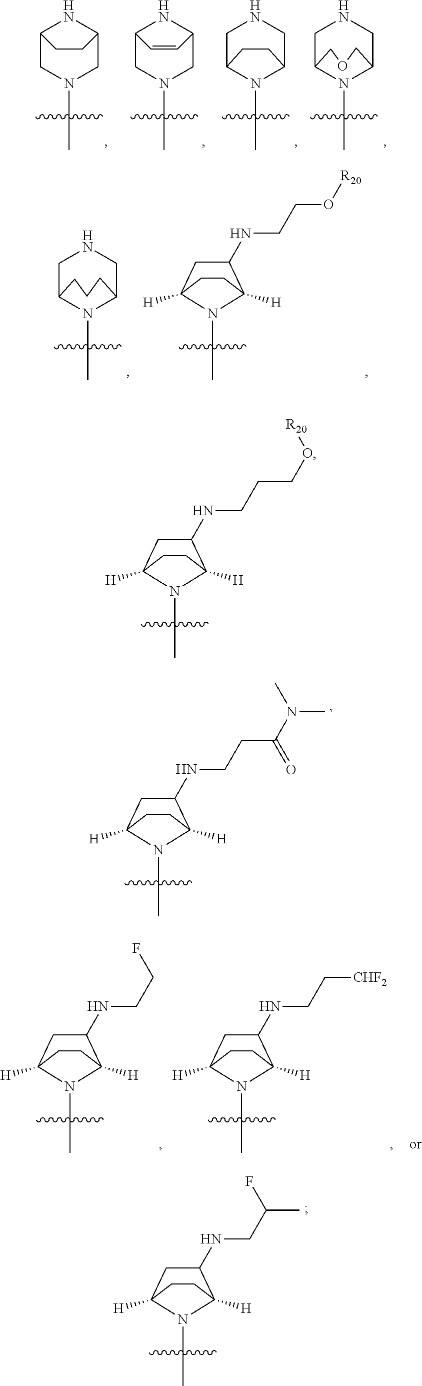

and
R₂₀ is methyl, —CHF₂, CF₃, —CH₂F, or —CD₃.

Aspect 39e: Provided herein are bridged compounds having the formula (IX) and pharmaceutically acceptable salts, tautomers, isotopologues, stereoisomers and prodrugs thereof,
wherein R₄ is F, Cl or methyl;
R₅ is —CF₃, —Cl, —OCF₃, —SCF₃, —CF₂CH₃, —CH₂CHF₂, —CN, —CHF₂, ethyl, isopropyl

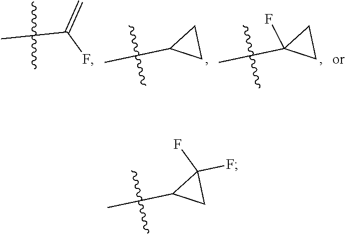

R₆ₐ is H, F, Cl or methyl;
R_{6b} is F, Cl or methyl;
R_{6d} is F, or Cl;
-L₁-R₈ is

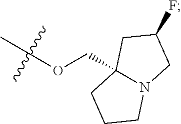 [SMILES: *OC[C@@]12CCCN1C[C@H](F)C2]

R₁₁ is

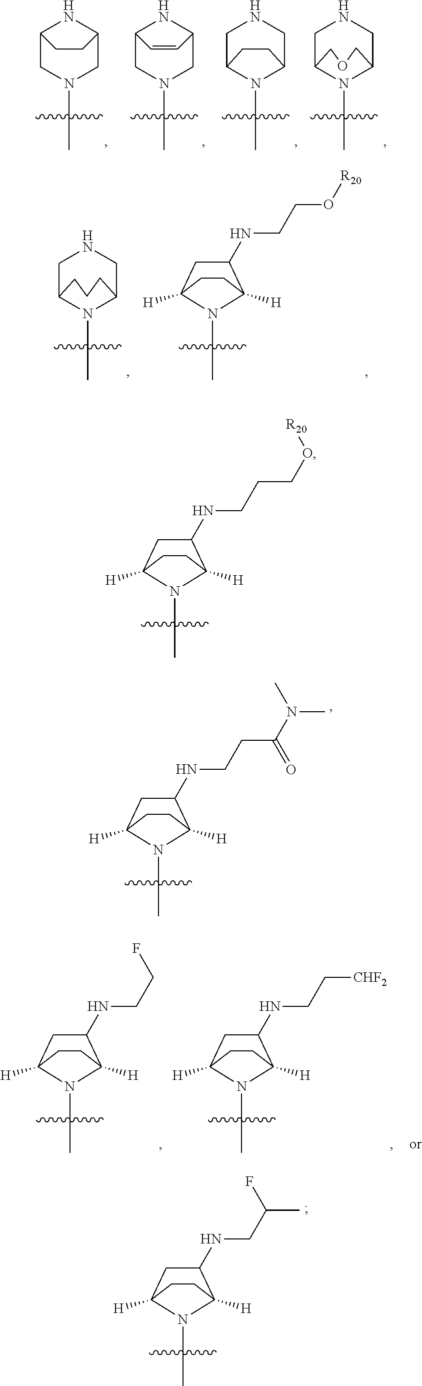

and
R₂₀ is methyl, —CHF₂, CF₃, —CH₂F, or —CD₃.

Aspect 39f: Provided herein are bridged compounds having formula (IX) and pharmaceutically acceptable salts, tautomers, isotopologues, stereoisomers and prodrugs thereof,
wherein R₄ is F, Cl or methyl;
R₅ is —CF₃;
R₆ₐ is H, F, Cl or methyl;
R_{6b} is F, Cl or methyl;
R_{6d} is H;
-L₁-R₈ is

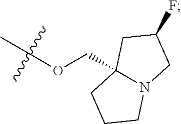 [SMILES: *OC[C@@]12CCCN1C[C@H](F)C2]

R₁₁ is

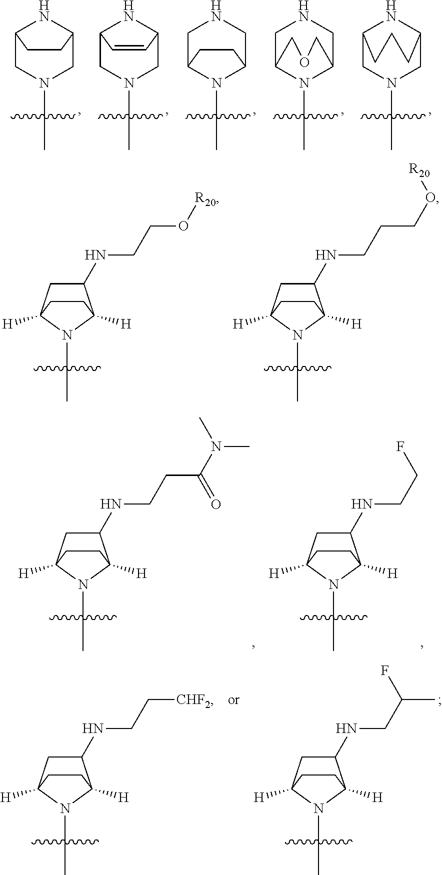

and
R₂₀ is methyl, —CHF₂, CF₃, —CH₂F, or —CD₃.

Aspect 39g: Provided herein are bridged compounds having formula (IX) and pharmaceutically acceptable salts, tautomers, isotopologues, stereoisomers and prodrugs thereof, wherein the bridged compound is selected from the compounds in Table 1, Table 2, and Table 6.

Aspect 40a: Provided herein are bridged compounds having the following formulas (Xa) or (Xb):

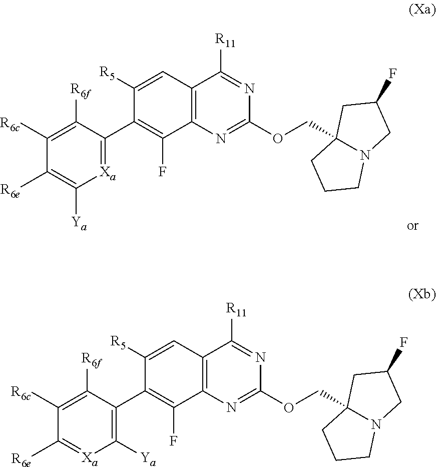

and pharmaceutically acceptable salts, tautomers, isotopologues, stereoisomers and prodrugs thereof, wherein Xₐ is N, or CH;
Yₐ is H, F, Cl, —NH₂ or —OH;
R₅ is —CF₃, —F, —Cl, —OCF₃, —SCF₃, —CF₂CH₃, —CH₂CHF₂, —CN, —CHF₂, ethyl, isopropyl

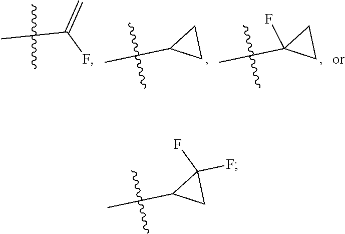

R_{6c} is H, F, Cl, methyl, ethyl, CHF₂, or CF₃;
R₆ₑ is H, F, or Cl;
R_{6f} is H, F, Cl, methyl, —CF₃, —CF₂CH₃, isopropyl, cyclopropyl, or methylcyclopropyl;
R₁₁ is

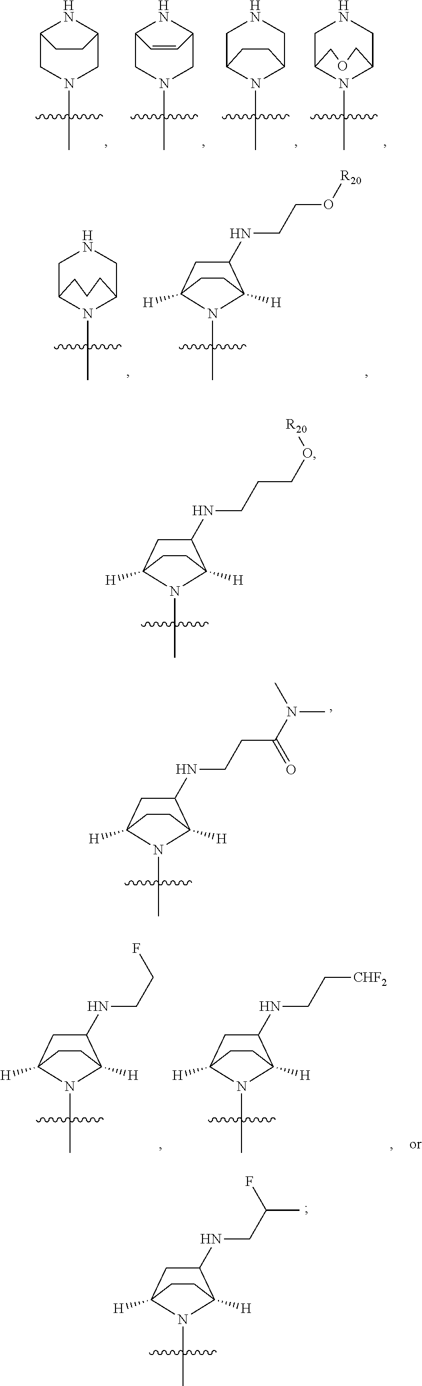

and
R₂₀ is methyl, —CHF₂, CF₃, —CH₂F, or —CD₃.

Aspect 40b: Provided herein are bridged compounds having formulas (Xa) or (Xb) and pharmaceutically acceptable salts, tautomers, isotopologues, stereoisomers and prodrugs thereof,
wherein Xₐ is N, or CH;
Yₐ is —NH₂ or —OH;
R₅ is —CF₃, —F, or —Cl;
R_{6c} is H, F, Cl, methyl, ethyl, CHF₂, or CF₃;
R₆ₑ is H, F, or Cl;
R_{6f} is F, Cl, methyl, —CF₃, —CF₂CH₃, isopropyl, cyclopropyl, or methylcyclopropyl;
R₁₁ is

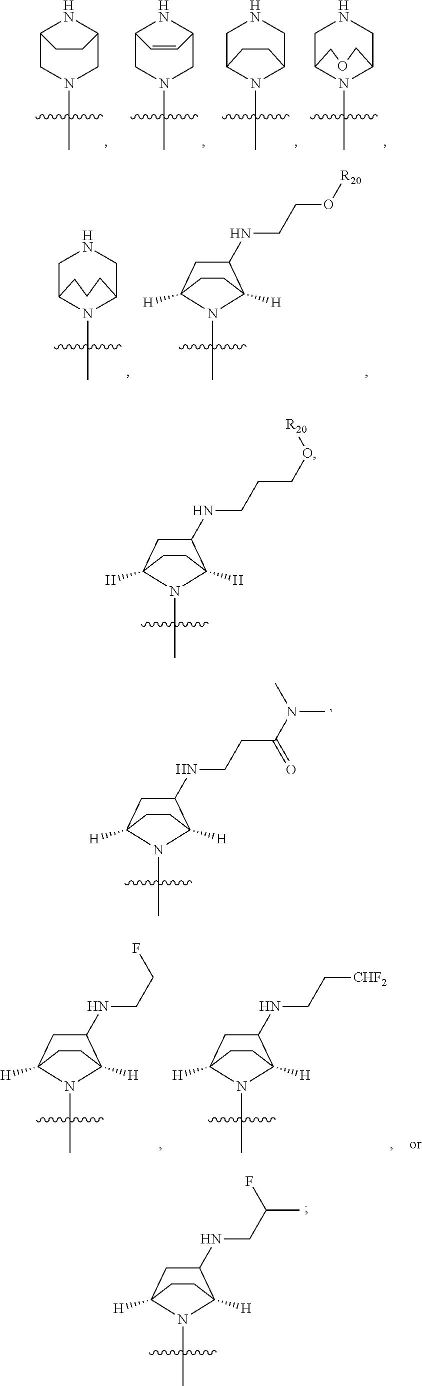

and
R₂₀ is methyl, —CHF₂, CF₃, —CH₂F, or —CD₃.

Aspect 40c: Provided herein are bridged compounds having formulas (Xa) or (Xb) and pharmaceutically acceptable salts, tautomers, isotopologues, stereoisomers and prodrugs thereof,
wherein Xₐ is N, or CH;
Yₐ is —NH₂ or —OH;
R₅ is —CF₃, —F, or —Cl;
R_{6c} is F, Cl, methyl, ethyl, CHF₂, or CF₃;
R₆ₑ is H;
R_{6f} is F, Cl, methyl, —CF₃, —CF₂CH₃, isopropyl, cyclopropyl, or methylcyclopropyl;
R₁₁ is

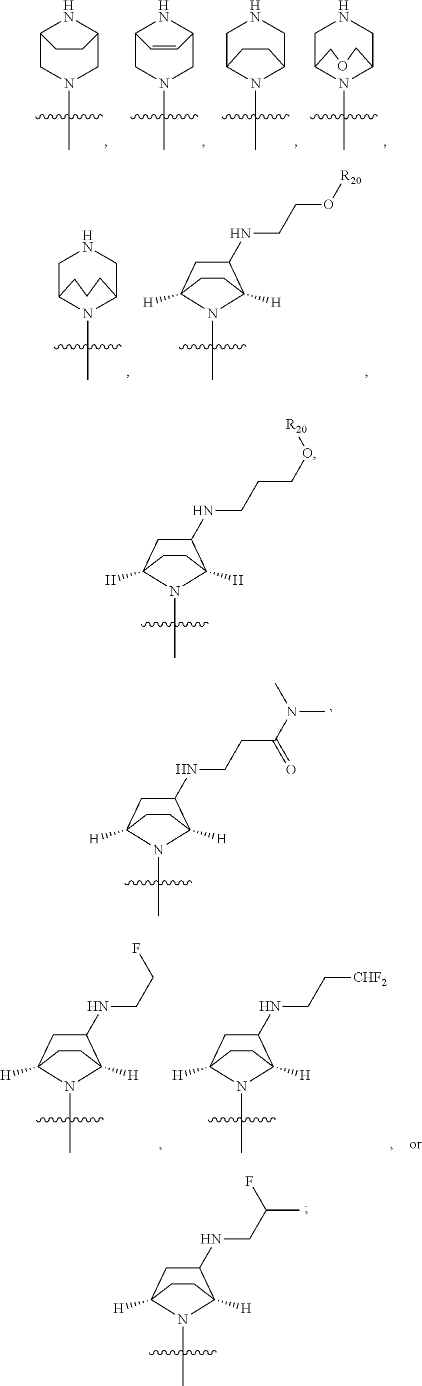

and
R₂₀ is methyl, —CHF₂, CF₃, —CH₂F, or —CD₃.

Aspect 40d: Provided herein are bridged compounds having formulas (Xa) or (Xb) and pharmaceutically acceptable salts, tautomers, isotopologues, stereoisomers and prodrugs thereof,
wherein Xₐ is N, or CH;
Yₐ is —NH₂ or —OH;
R₅ is —CF₃, —F, or —Cl;
R_{6c} is F, Cl, methyl, ethyl, CHF₂, or CF₃;
R₆ₑ is H;
R_{6f} is F, Cl, —CF₃;
R₁₁ is

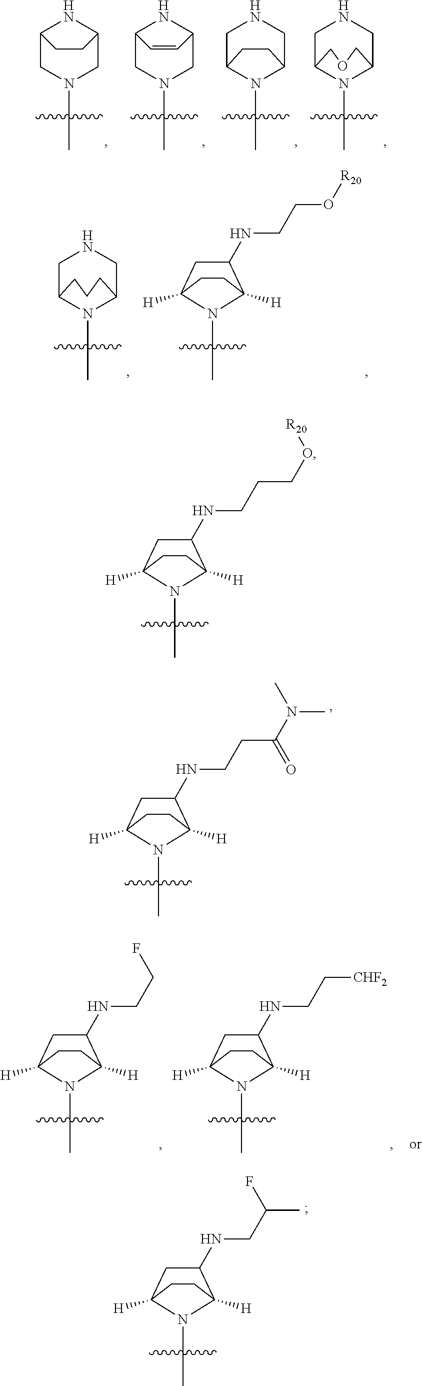

and
R₂₀ is methyl, —CHF₂, CF₃, —CH₂F, or —CD₃.

Aspect 40e: Provided herein are bridged compounds having formula (Xa) and pharmaceutically acceptable salts, tautomers, isotopologues, stereoisomers and prodrugs thereof,
wherein Xₐ is N, or CH;
Yₐ is —NH₂;
R₅ is —CF₃, —F, or —Cl;
R_{6c} is F, Cl, methyl, ethyl, CHF₂, or CF₃;
R₆ₑ is H;
R_{6f} is —CF₃;
R₁₁ is

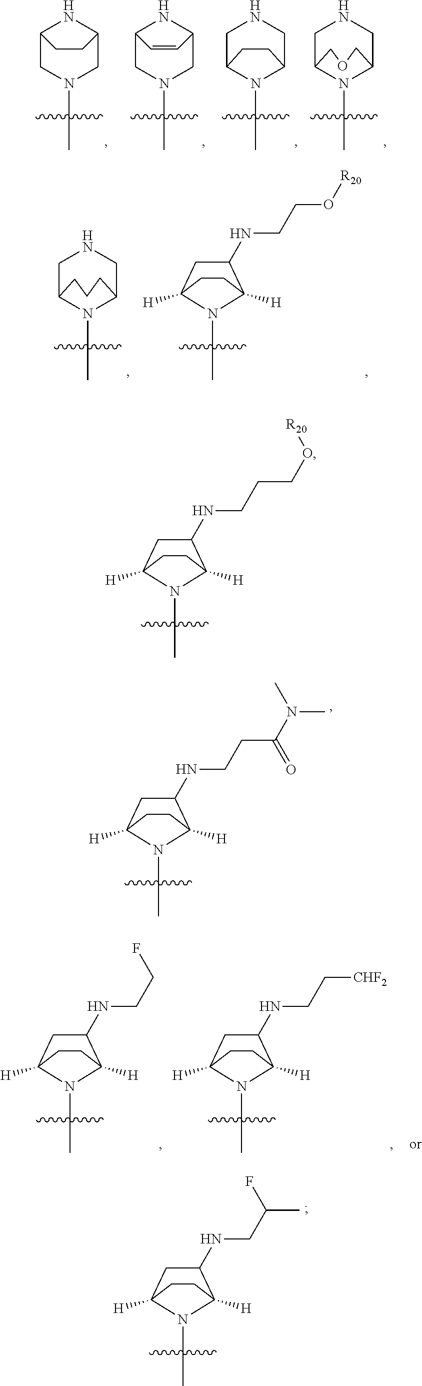

and
R₂₀ is methyl, —CHF₂, CF₃, —CH₂F, or —CD₃.

Aspect 40f: Provided herein are bridged compounds having formulas (IX), (Xa) or (Xb) and pharmaceutically acceptable salts, tautomers, isotopologues, stereoisomers and prodrugs thereof, wherein the compound is

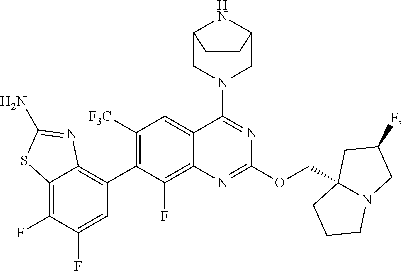 [SMILES: Nc1nc2c(-c3c(C(F)(F)F)cc4c(N5CC6CCC(C5)N6)nc(OC[C@@]56CCCN5C[C@H](F)C6)nc4c3F)cc(F)c(F)c2s1]

4-(4-(3,8-
diazabicyclo[3.2.1]octan-3-yl)-8-fluoro-2-
(((2R,7aS)-2-fluorotetrahydro-1H-pyrrolizin-
7a(5H)-yl)methoxy)-6-
(trifluoromethyl)quinazolin-7-yl)-6,7-
difluorobenzo[d]thiazol-2-amine

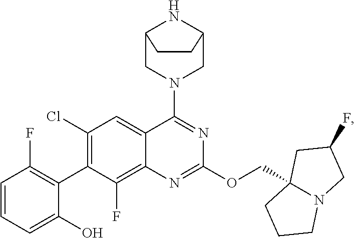 [SMILES: Oc1cccc(F)c1-c1c(Cl)cc2c(N3CC4CCC(C3)N4)nc(OC[C@@]34CCCN3C[C@H](F)C4)nc2c1F]

2-(4-(3,8-
diazabicyclo[3.2.1]octan-3-yl)-6-chloro-8-fluoro-
2-(((2R,7aS)-2-fluorotetrahydro-1H-pyrrolizin-
7a(5H)-yl)methoxy)quinazolin-7-yl)-3-
fluorophenol

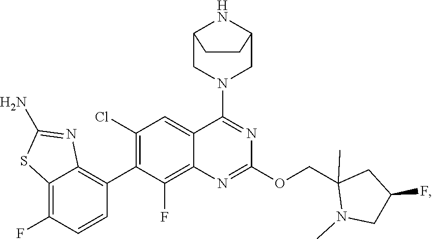 [SMILES: CN1C[C@H](F)CC1(C)COc1nc(N2CC3CCC(C2)N3)c2cc(Cl)c(-c3ccc(F)c4sc(N)nc34)c(F)c2n1]

4-(4-
(3,8-diazabicyclo[3.2.1]octan-3-yl)-6-chloro-8-
fluoro-2-(((4R)-4-fluoro-1,2-dimethylpyrrolidin-
2-yl)methoxy)quinazolin-7-yl)-7-
fluorobenzo[d]thiazol-2-amine

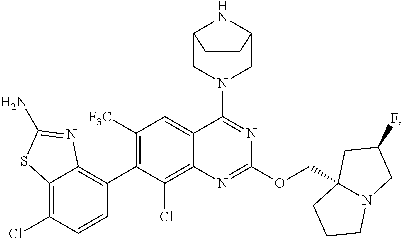 [SMILES: Nc1nc2c(-c3c(C(F)(F)F)cc4c(N5CC6CCC(C5)N6)nc(OC[C@@]56CCCN5C[C@H](F)C6)nc4c3Cl)ccc(Cl)c2s1]

4-(4-(3,8-
diazabicyclo[3.2.1]octan-3-yl)-8-chloro-2-
(((2R,7aS)-2-fluorotetrahydro-1H-pyrrolizin-
7a(5H)-yl)methoxy)-6-
(trifluoromethyl)quinazolin-7-yl)-7-
chlorobenzo[d]thiazol-2-amine

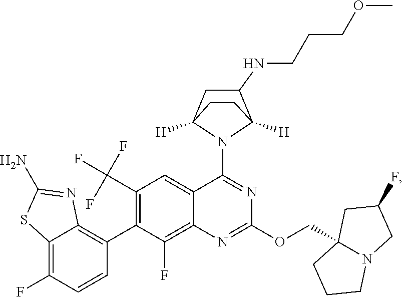 [SMILES: COCCCNC1C[C@H]2CC[C@@H]1N2c1nc(OC[C@@]23CCCN2C[C@H](F)C3)nc2c(F)c(-c3ccc(F)c4sc(N)nc34)c(C(F)(F)F)cc12]

7-fluoro-4-
(8-fluoro-2-(((2R,7aS)-2-fluorotetrahydro-1H-
pyrrolizin-7a(5H)-yl)methoxy)-4-((1S,4R)-2-((3-
methoxypropyl)amino)-7-
azabicyclo[2.2.1]heptan-7-yl)-6-
(trifluoromethyl)quinazolin-7-yl)benzo[d]thiazol-
2-amine

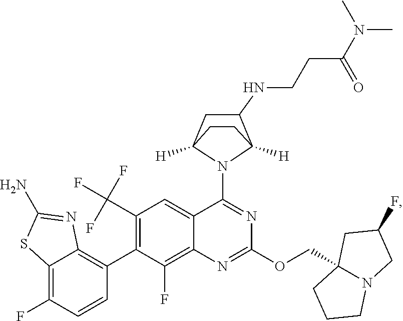 [SMILES: CN(C)C(=O)CCNC1C[C@H]2CC[C@@H]1N2c1nc(OC[C@@]23CCCN2C[C@H](F)C3)nc2c(F)c(-c3ccc(F)c4sc(N)nc34)c(C(F)(F)F)cc12]

3-(((1S,4R)-7-(7-
(2-amino-7-fluorobenzo[d]thiazol-
4-yl)-8-fluoro-2-(((2R,7aS)-2-fluorotetrahydro-
1H-pyrrolizin-7a(5H)-yl)methoxy)-6-
(trifluoromethyl)quinazolin-4-yl)-7-
azabicyclo[2.2.1]heptan-2-yl)amino)-N,N-
dimethylpropanamide

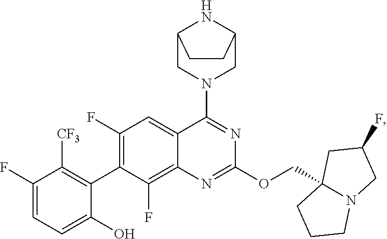 [SMILES: Oc1ccc(F)c(C(F)(F)F)c1-c1c(F)cc2c(N3CC4CCC(C3)N4)nc(OC[C@@]34CCCN3C[C@H](F)C4)nc2c1F]

2-(4-(3,8-diazabicyclo[3.2.1]octan-3-yl)-6,8-
difluoro-2-(((2R,7aS)-2-fluorotetrahydro-1H-
pyrrolizin-7a(5H)-yl)methoxy)quinazolin-7-yl)-4-
fluoro-3-(trifluoromethyl)phenol

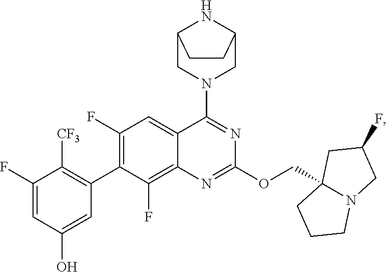 [SMILES: Oc1cc(F)c(C(F)(F)F)c(-c2c(F)cc3c(N4CC5CCC(C4)N5)nc(OC[C@@]45CCCN4C[C@H](F)C5)nc3c2F)c1]

3-(4-(3,8-diazabicyclo[3.2.1]octan-3-yl)-6,8-
difluoro-2-(((2R,7aS)-2-fluorotetrahydro-1H-
pyrrolizin-7a(5H)-yl)methoxy)quinazolin-7-yl)-5-
fluoro-4-(trifluoromethyl)phenol

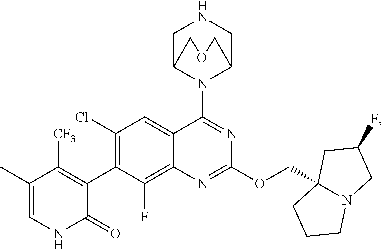 [SMILES: Cc1c[nH]c(=O)c(-c2c(Cl)cc3c(N4C5CNCC4COC5)nc(OC[C@@]45CCCN4C[C@H](F)C5)nc3c2F)c1C(F)(F)F]

3-(4-(3-oxa-7,9-diazabicyclo[3.3.1]nonan-9-yl)-6-
chloro-8-fluoro-2-(((2R,7aS)-2-fluorotetrahydro-
1H-pyrrolizin-7a(5H)-yl)methoxy)quinazolin-7-
yl)-5-methyl-4-(trifluoromethyl)pyridin-2(1H)-
one

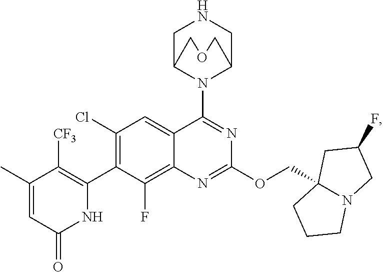 [SMILES: Cc1cc(=O)[nH]c(-c2c(Cl)cc3c(N4C5CNCC4COC5)nc(OC[C@@]45CCCN4C[C@H](F)C5)nc3c2F)c1C(F)(F)F]

6-(4-(3-oxa-7,9-diazabicyclo[3.3.1]nonan-9-yl)-6-
chloro-8-fluoro-2-(((2R,7aS)-2-fluorotetrahydro-
1H-pyrrolizin-7a(5H)-yl)methoxy)quinazolin-7-
yl)-4-methyl-5-(trifluoromethyl)pyridin-2(1H)-
one

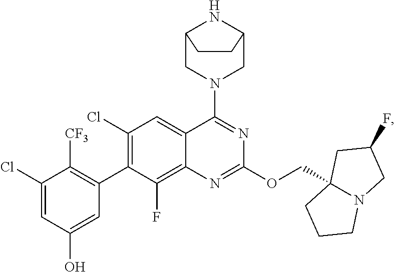 [SMILES: Oc1cc(Cl)c(C(F)(F)F)c(-c2c(Cl)cc3c(N4CC5CCC(C4)N5)nc(OC[C@@]45CCCN4C[C@H](F)C5)nc3c2F)c1]

3-(4-(3,8-diazabicyclo[3.2.1]octan-3-yl)-6-chloro-
8-fluoro-2-(((2R,7aS)-2-fluorotetrahydro-1H-
pyrrolizin-7a(5H)-yl)methoxy)quinazolin-7-yl)-5-
fluoro-4-(trifluoromethyl)phenol

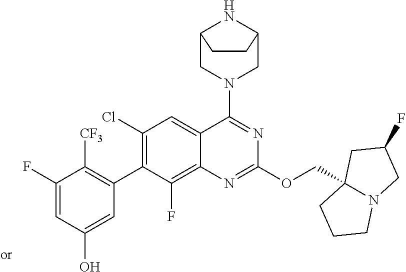 [SMILES: Oc1cc(F)c(C(F)(F)F)c(-c2c(Cl)cc3c(N4CC5CCC(C4)N5)nc(OC[C@@]45CCCN4C[C@H](F)C5)nc3c2F)c1]

3-(4-(3,8-diazabicyclo[3.2.1]octan-3-yl)-6-chloro-
8-fluoro-2-(((2R,7aS)-2-fluorotetrahydro-1H-
pyrrolizin-7a(5H)-yl)methoxy)quinazolin-7-yl)-5-
chloro-4-(trifluoromethyl)phenol.

Aspect 40g: Provided herein are bridged compounds having formulas (Xa) or (Xb) and pharmaceutically acceptable salts, tautomers, isotopologues, stereoisomers and prodrugs thereof, wherein the bridged compound is selected from the compounds in Table 1, Table 2, and Table 6.

Aspect 41a: Provided herein are bridged compounds having the following formula (XI):

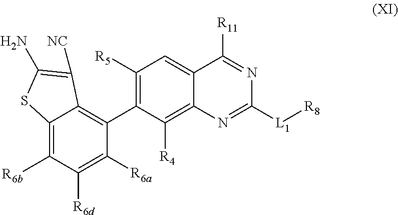

and pharmaceutically acceptable salts, tautomers, isotopologues, stereoisomers and prodrugs thereof, wherein R₄ is F, Cl or methyl;

R₅ is —CF₃, —Cl, —OCF₃, —SCF₃, —CF₂CH₃, —CH₂CHF₂, —CN, —CHF₂, ethyl, isopropyl

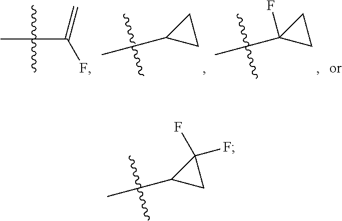

R₆ₐ is H, F, Cl or methyl;
R_{6b} is H, F, Cl or methyl;
R_{6d} is H, F, Cl or methyl;
-L₁-R₈ is

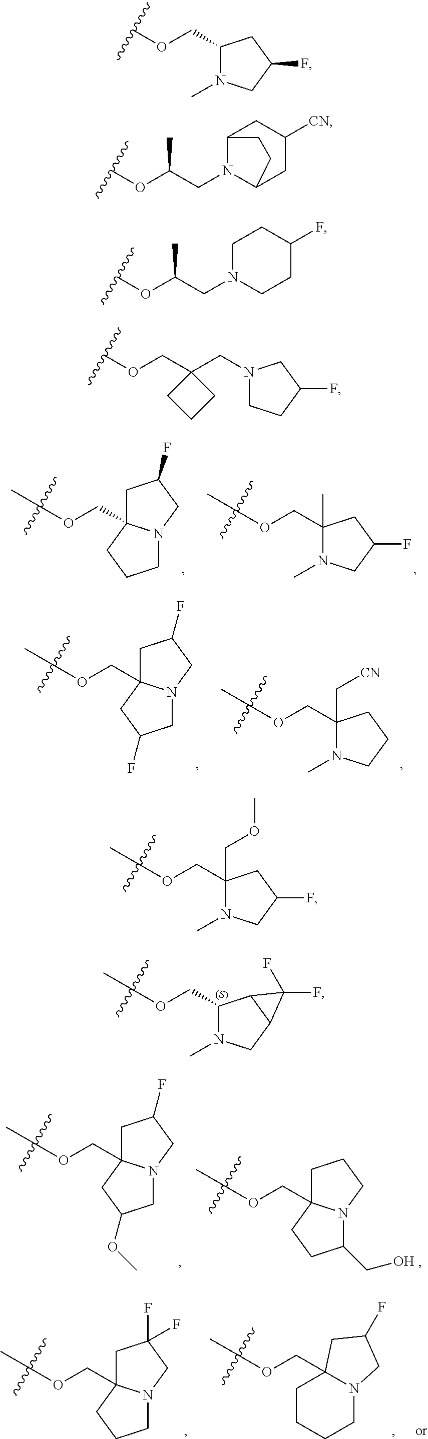

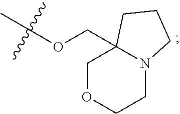 [SMILES: *OCC12CCCN1CCOC2]

R₁₁ is

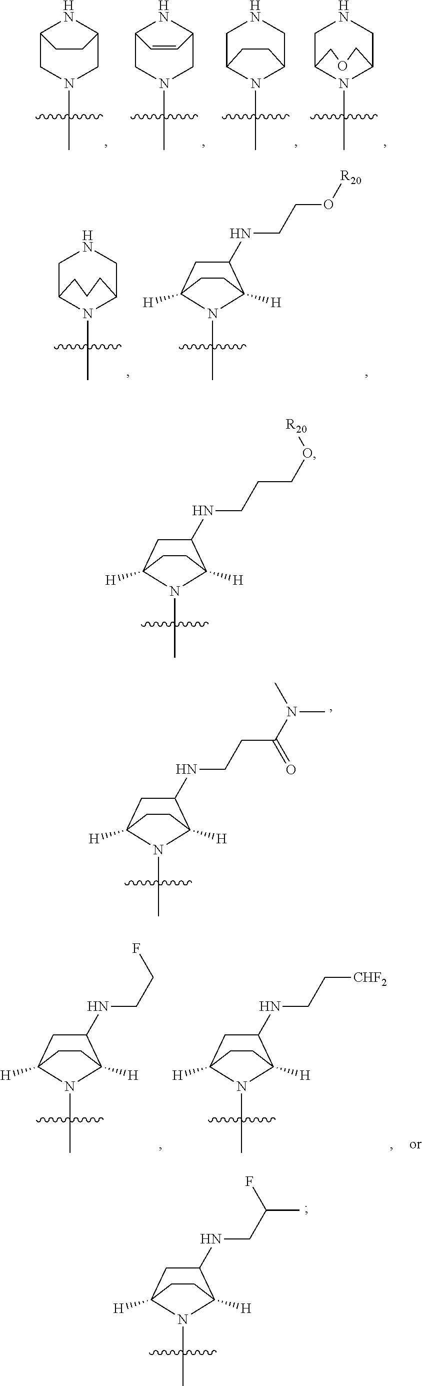

and
R₂₀ is methyl, —CHF₂, CF₃, —CH₂F, or —CD₃.

Aspect 41b: Provided herein are bridged compounds having the following formula (XII):

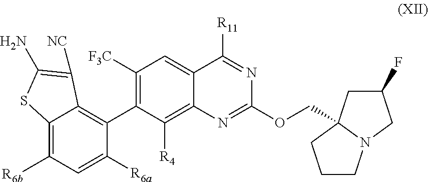

and pharmaceutically acceptable salts, tautomers, isotopologues, stereoisomers and prodrugs thereof,
wherein R₄ is F, Cl or methyl;
R₆ₐ is H, F, Cl or methyl;
R_{6b} is F, Cl or methyl;
R₁₁ is

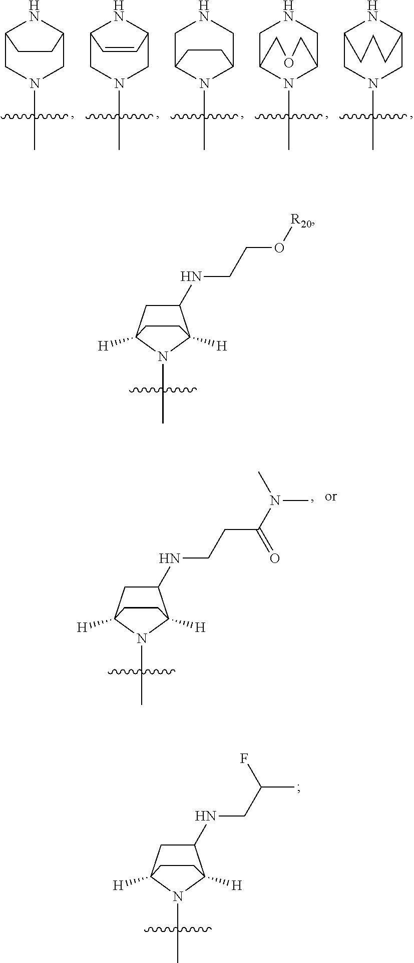

and
R₂₀ is methyl, —CHF₂, CF₃, —CH₂F, or —CD₃.

Aspect 42a: Provided herein are bridged compounds having the following formula (XIII):

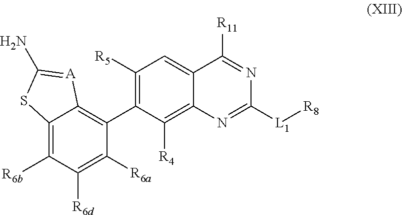

and pharmaceutically acceptable salts, tautomers, isotopologues, stereoisomers and prodrugs thereof,
wherein A is N or C—CN;
R₄ is H, F, Cl or methyl;
R₅ is —CF₃, —Cl, —OCF₃, —OCF₂Cl, —SCF₃, —H, —F, —Br, —I, —CF₂CH₃, —CH₂CHF₂, —CH₂CF₃, —CN, —CHF₂, ethyl, isopropyl

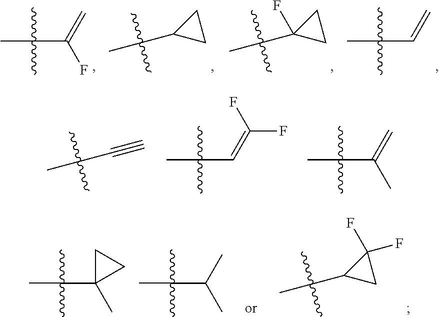

R₆ₐ is H, F, Cl or methyl;
R_{6b} is H, F, Cl or methyl;
R_{6d} is H, F, Cl or methyl;
-L₁- is —O—;
R₈ is unsubstituted or substituted alkyl, unsubstituted or substituted cycloalkyl, unsubstituted or substituted cycloalkylalkyl, unsubstituted or substituted aryl, unsubstituted or substituted aralkyl, unsubstituted or substituted heterocyclyl, unsubstituted or substituted heterocyclylalkyl, unsubstituted or substituted heteroaryl, unsubstituted or substituted heteroaralkyl, unsubstituted or substituted spirocyclic ring or unsubstituted or substituted -alkyl-spirocyclic ring,
R₁₁ is

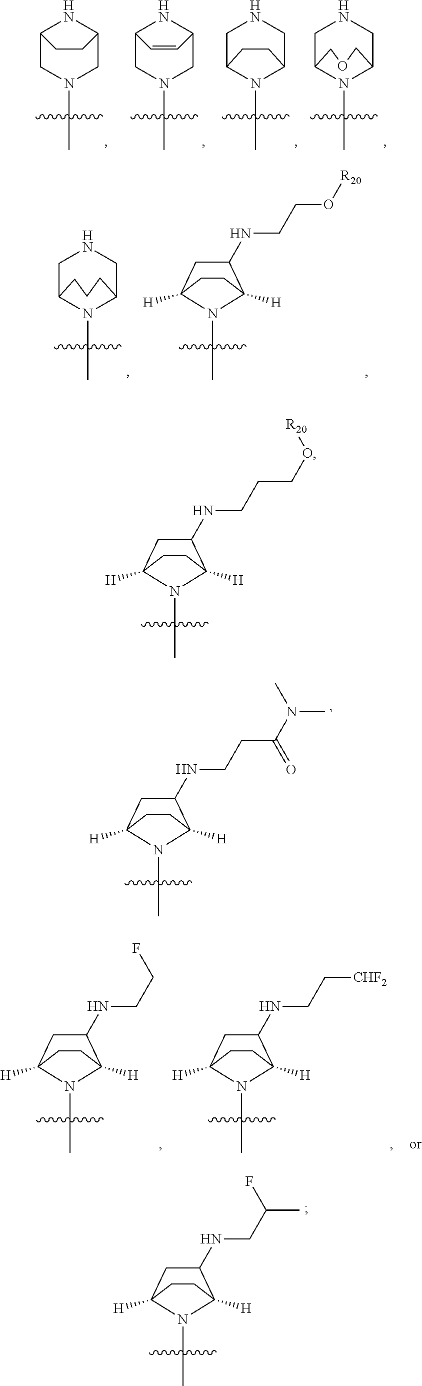

and
R₂₀ is methyl, —CHF₂, CF₃, —CH₂F, or —CD₃.

Aspect 42b: Provided herein are bridged compounds having the formula (XIII) and pharmaceutically acceptable salts, tautomers, isotopologues, stereoisomers and prodrugs thereof, wherein A is N or C—CN;
R₄ is F, Cl;
R₅ is —CF₃;
R₆ₐ is H;
R_{6b} is F;
R_{6d} is H;
-L₁- is —O—;
R₈ is unsubstituted or substituted alkyl, unsubstituted or substituted cycloalkyl, unsubstituted or substituted cycloalkylalkyl, unsubstituted or substituted aryl, unsubstituted or substituted aralkyl, unsubstituted or substituted heterocyclyl, unsubstituted or substituted heterocyclylalkyl, unsubstituted or substituted heteroaryl, unsubstituted or substituted heteroaralkyl, unsubstituted or substituted spirocyclic ring or unsubstituted or substituted -alkyl-spirocyclic ring;
R₁₁ is

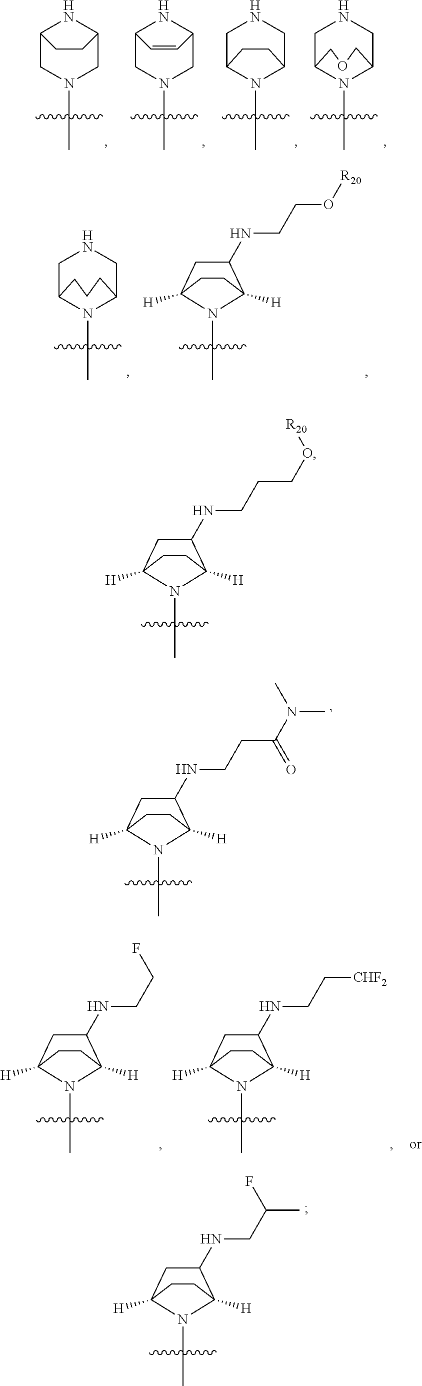

and
R₂₀ is methyl, —CHF₂, CF₃, —CH₂F, or —CD₃.

Aspect 42c: Provided herein are bridged compounds having the formula (XIII) and pharmaceutically acceptable salts, tautomers, isotopologues, stereoisomers and prodrugs thereof,
wherein A is N or C—CN;
R₄ is F;
R₅ is —CF₃;
R₆ₐ is H;
R_{6b} is F;
R_{6d} is H;
-L₁- is —O—;
R₈ is unsubstituted or substituted alkyl, unsubstituted or substituted cycloalkyl, unsubstituted or substituted cycloalkylalkyl, unsubstituted or substituted aryl, unsubstituted or substituted aralkyl, unsubstituted or substituted heterocyclyl, unsubstituted or substituted heterocyclylalkyl, unsubstituted or substituted heteroaryl, unsubstituted or substituted heteroaralkyl, unsubstituted or substituted spirocyclic ring, or unsubstituted or substituted -alkyl-spirocyclic ring;
R₁₁ is

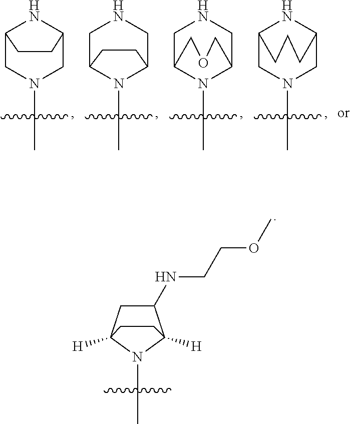

Aspect 42d: Provided herein are bridged compounds having the formula (XIII) and pharmaceutically acceptable salts, tautomers, isotopologues, stereoisomers and prodrugs thereof,
wherein A is N or C—CN;
R₄ is F, Cl or methyl;
R₅ is —CF₃, —Cl, —OCF₃, —OCF₂Cl, —SCF₃, —H, —F, —Br, —I, —CF₂CH₃, —CH₂CHF₂, —CH₂CF₃, —CN, —CHF₂, ethyl, isopropyl,

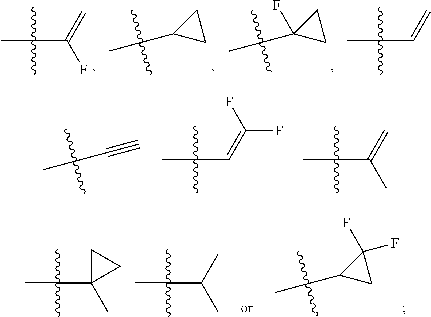

R₆ₐ is H, F, Cl or methyl;
R_{6b} is F, Cl or methyl;
R_{6d} is H or F;
-L₁-R₈ is

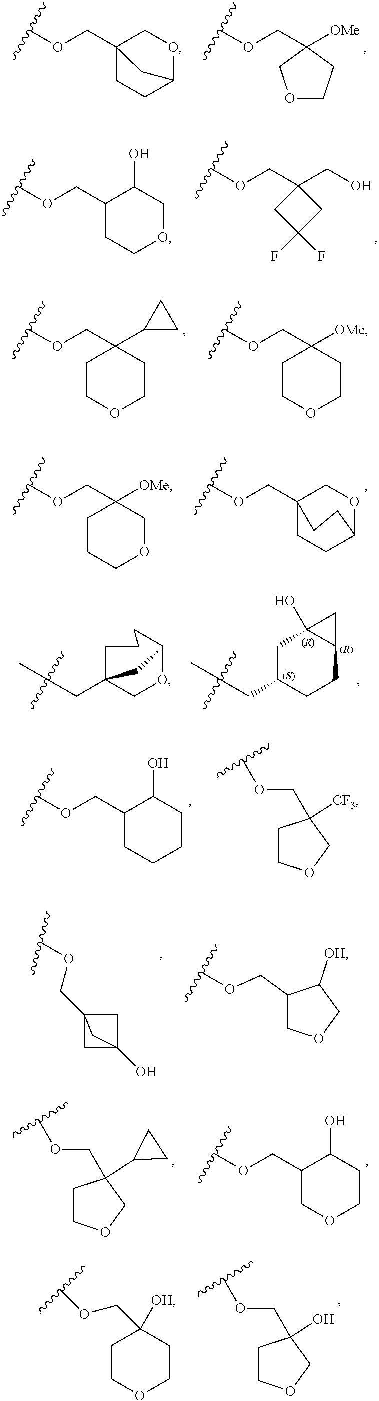

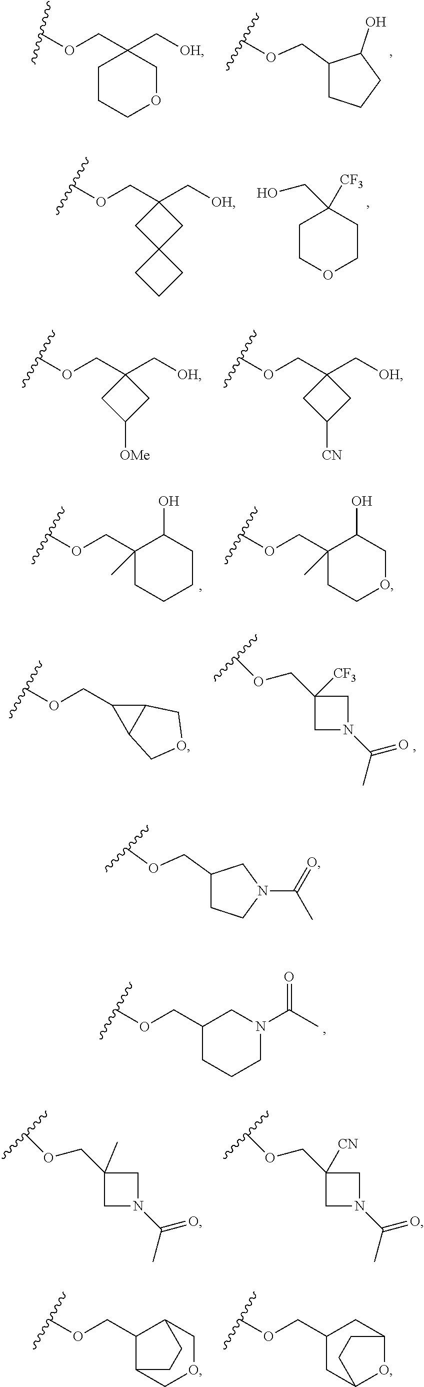

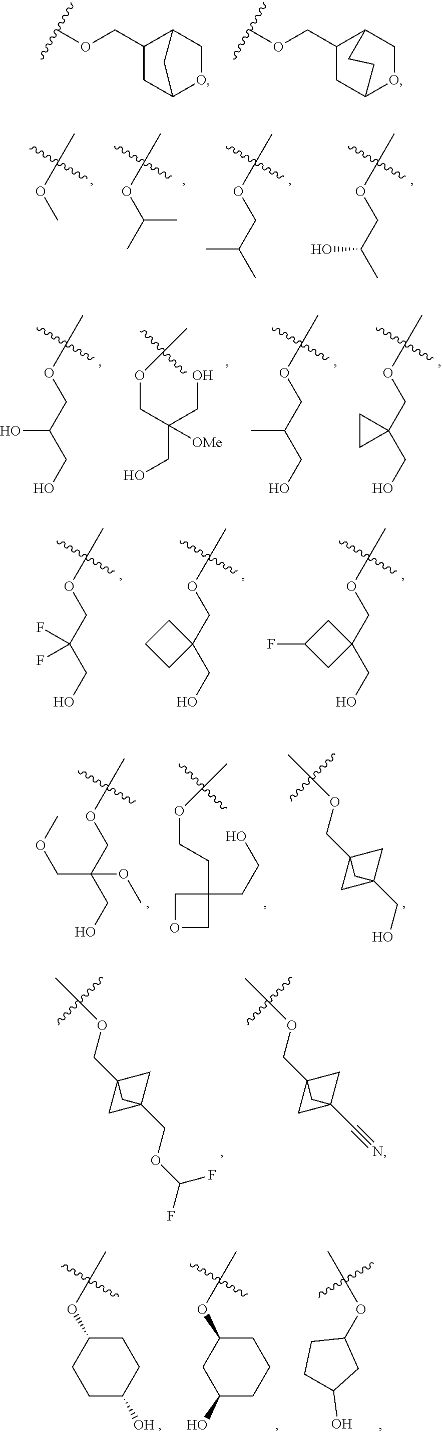

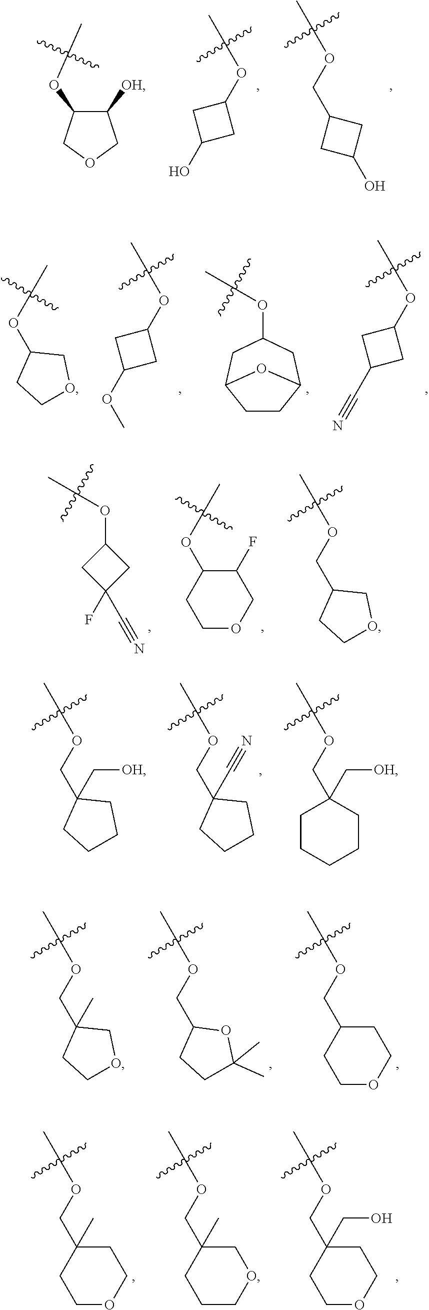

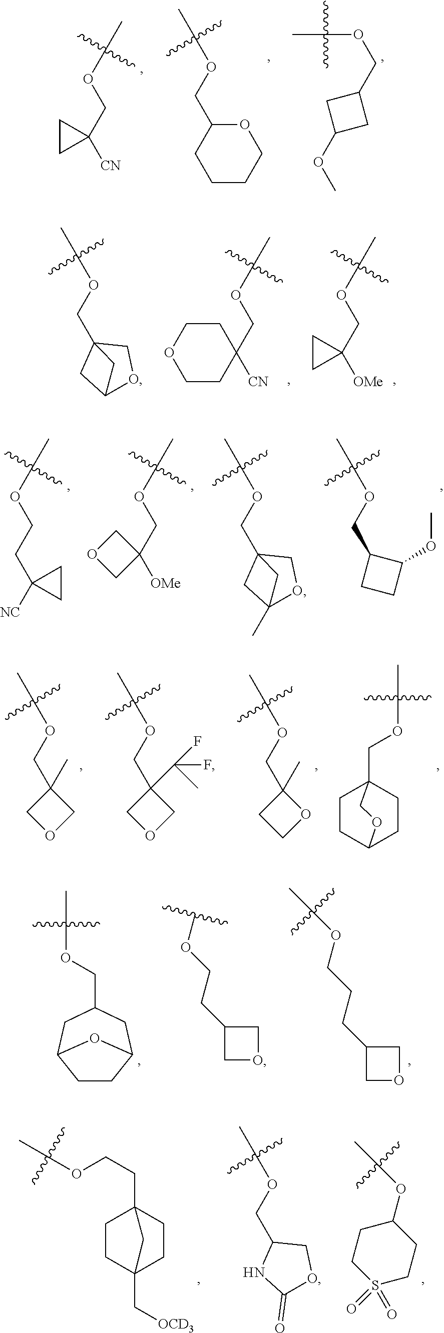

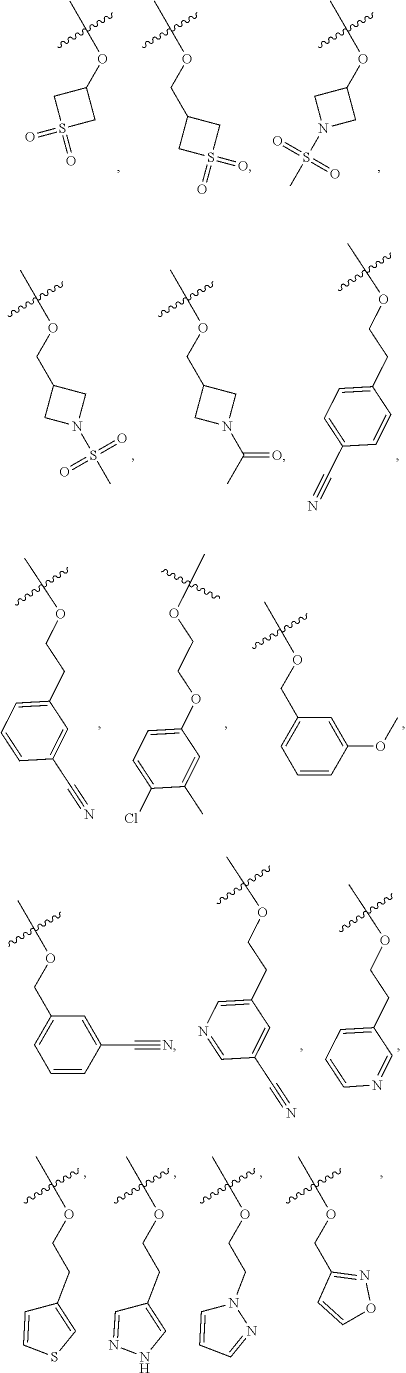

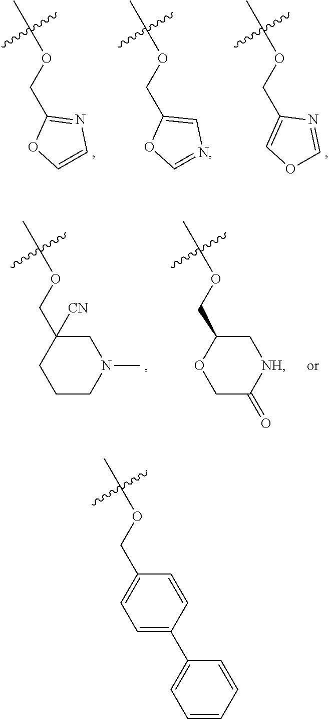 [SMILES: *OCc1ncco1 ; *OCc1cnco1 ; *OCc1cocn1 ; *OCC1(C#N)CCCN(C)C1 ; *OC[C@H]1CNC(=O)CO1 ; *OCc1ccc(-c2ccccc2)cc1]

R₁₁ is

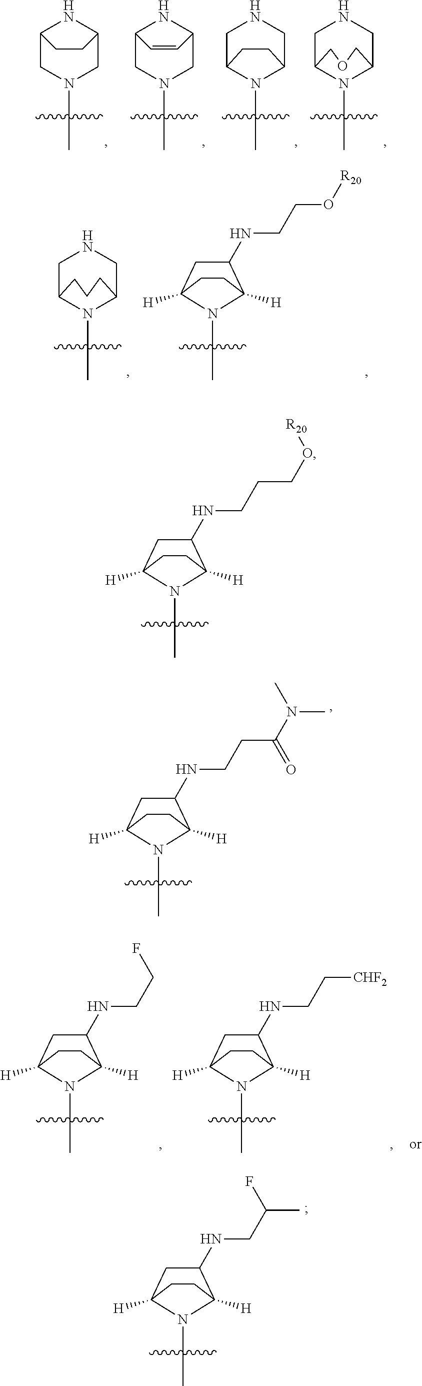

and
R₂₀ is methyl, —CHF₂, CF₃, —CH₂F, or —CD₃.

In one embodiment, A is N. In another embodiment, A is C—CN.

Aspect 42e: Provided herein are bridged compounds having the formula (XIII) and pharmaceutically acceptable salts, tautomers, isotopologues, stereoisomers and prodrugs thereof,
wherein A is N or C—CN;
R₄ is F;
R₅ is —CF₃;
R₆ₐ is H;
R_{6b} is F;
R_{6d} is H;
-L₁-R₈ is

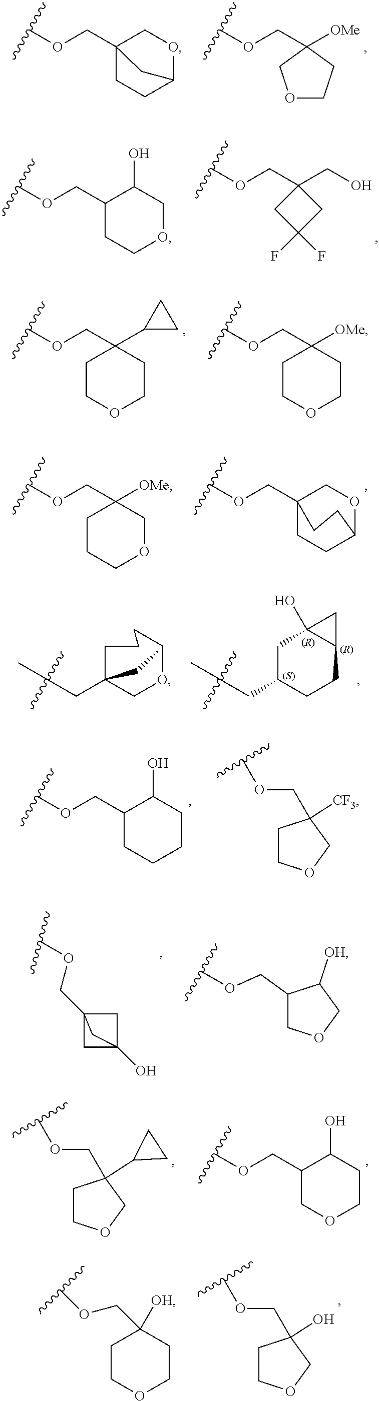

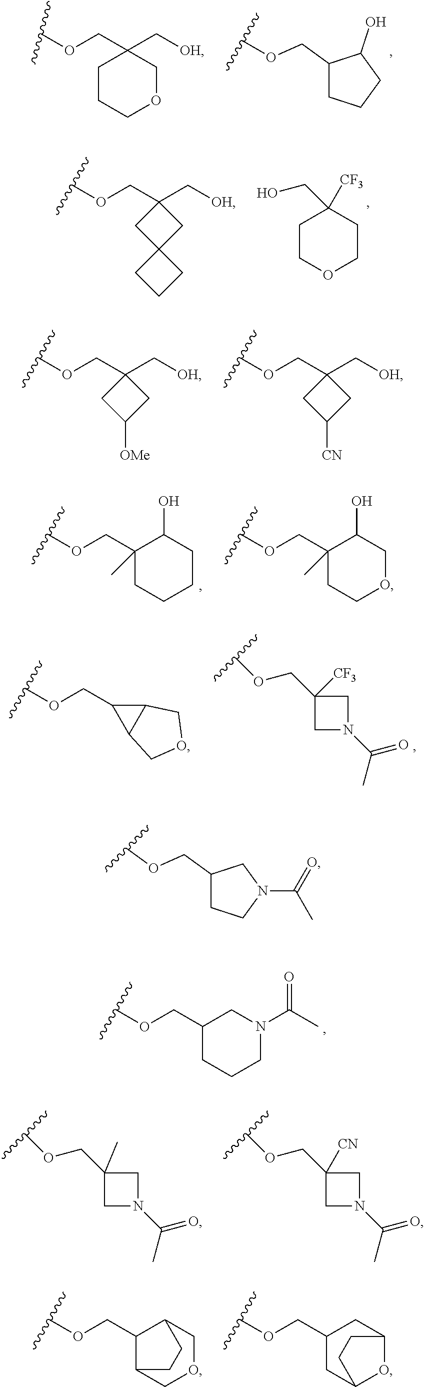

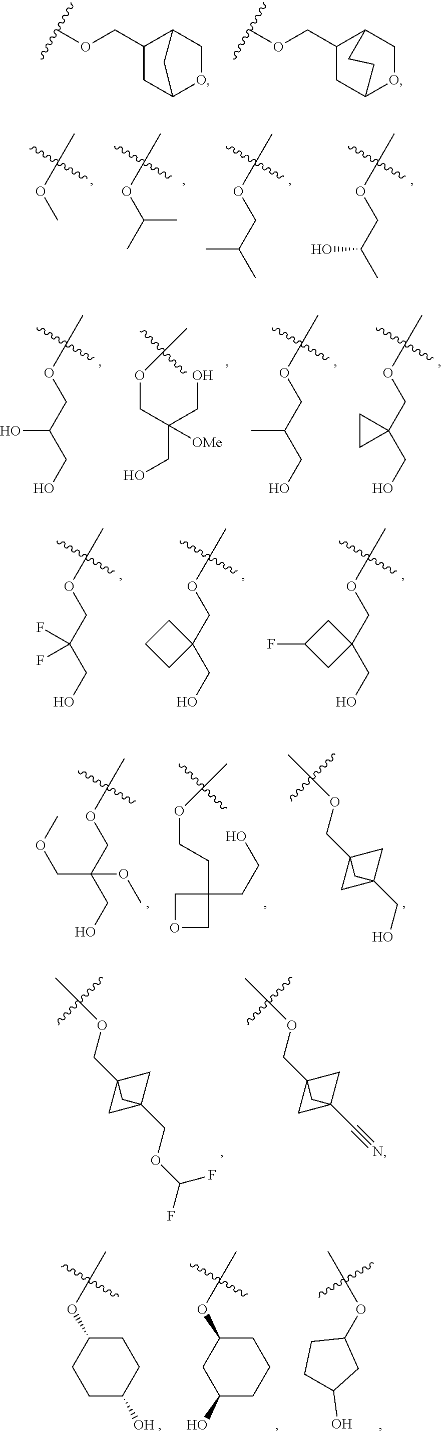

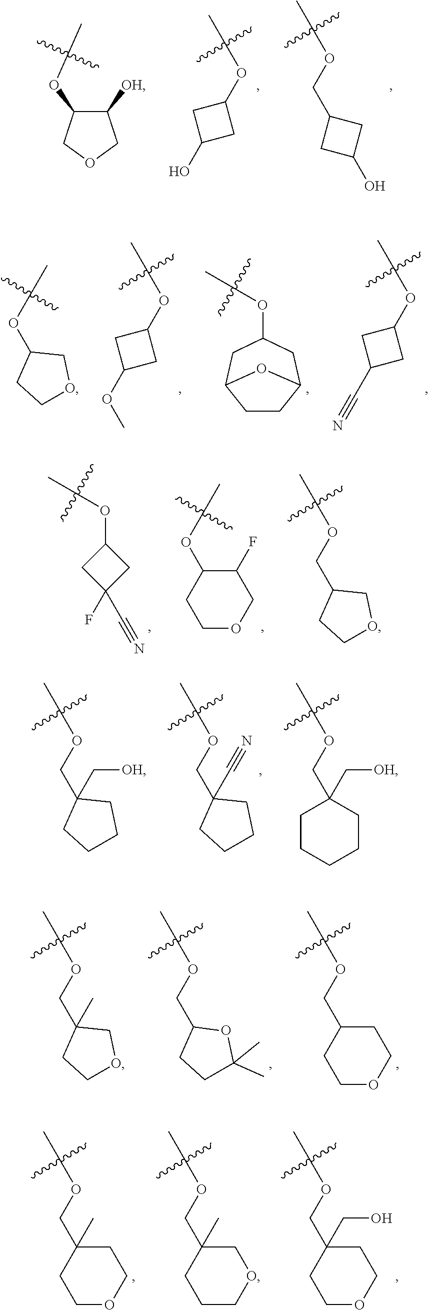

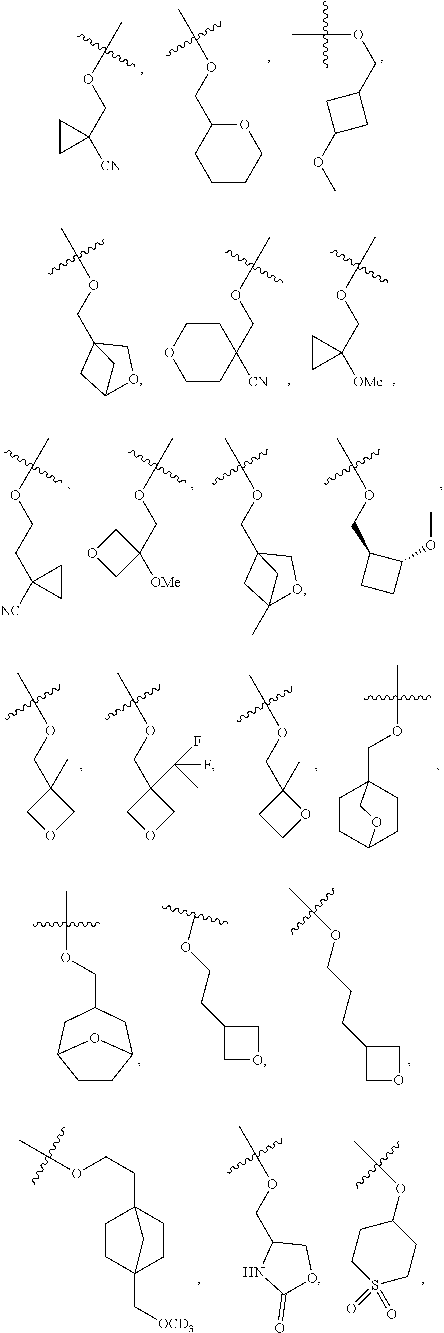

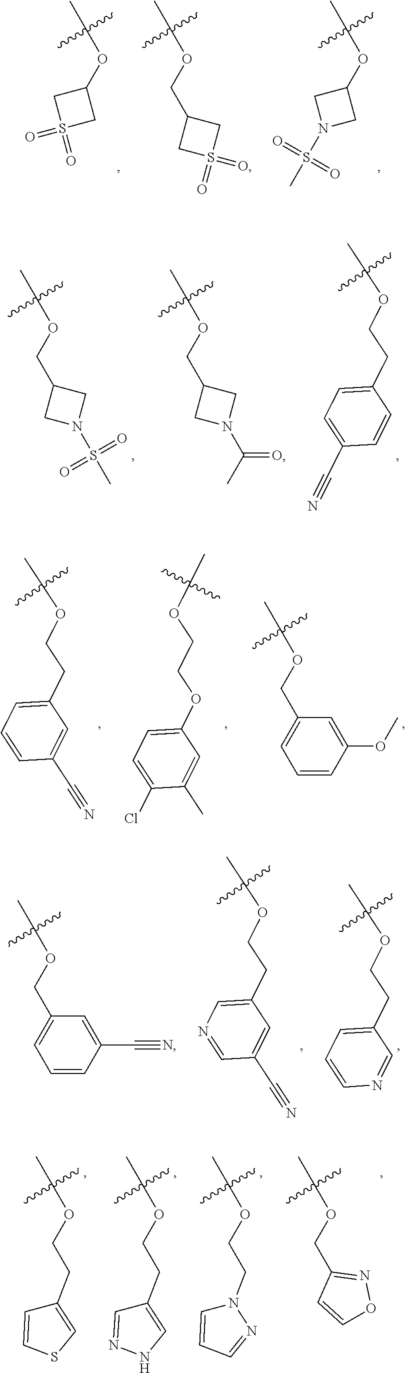

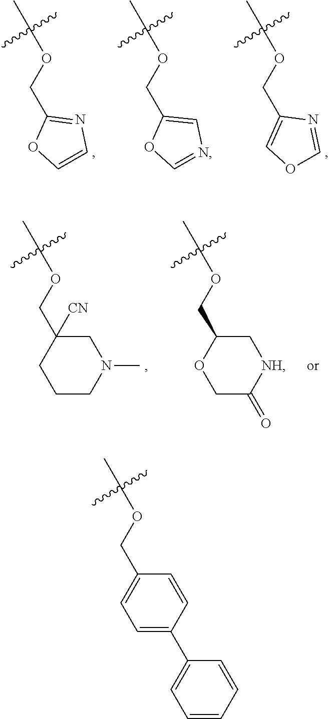 [SMILES: *OCc1ncco1 ; *OCc1cnco1 ; *OCc1cocn1 ; *OCC1(C#N)CCCN(C)C1 ; *OC[C@H]1CNC(=O)CO1 ; *OCc1ccc(-c2ccccc2)cc1]

R₁₁ is

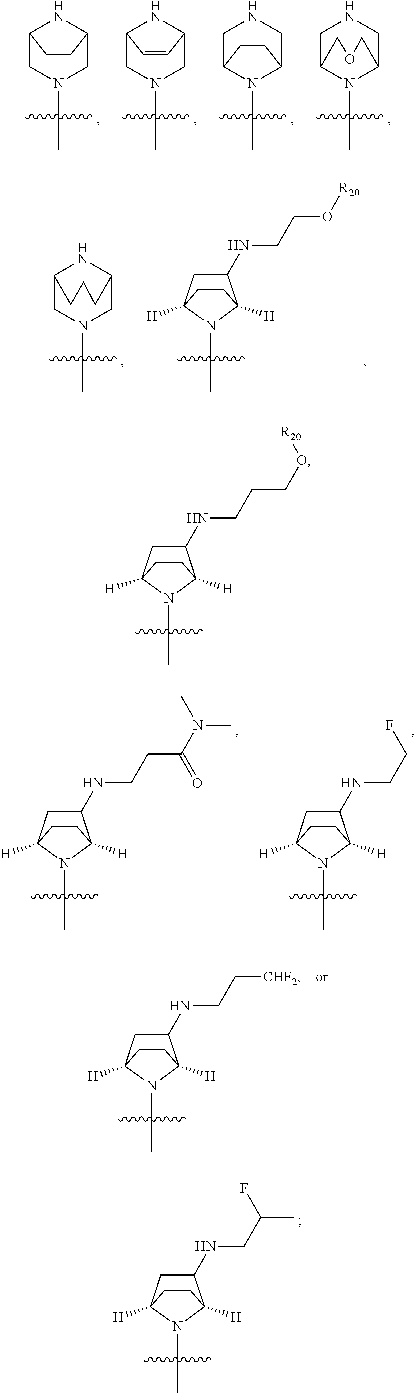

and
R₂₀ is methyl, —CHF₂, CF₃, —CH₂F, or —CD₃.

In one embodiment, A is N. In another embodiment, A is C—CN.

Aspect 42f: Provided herein are bridged compounds having formula (XIII) and pharmaceutically acceptable salts, tautomers, isotopologues, stereoisomers and prodrugs thereof,
wherein A is N or C—CN;
R₄ is F;
R₅ is —CF₃;
R₆ₐ is H;
R_{6b} is F;
R_{6d} is H;
L₁-R₈ is

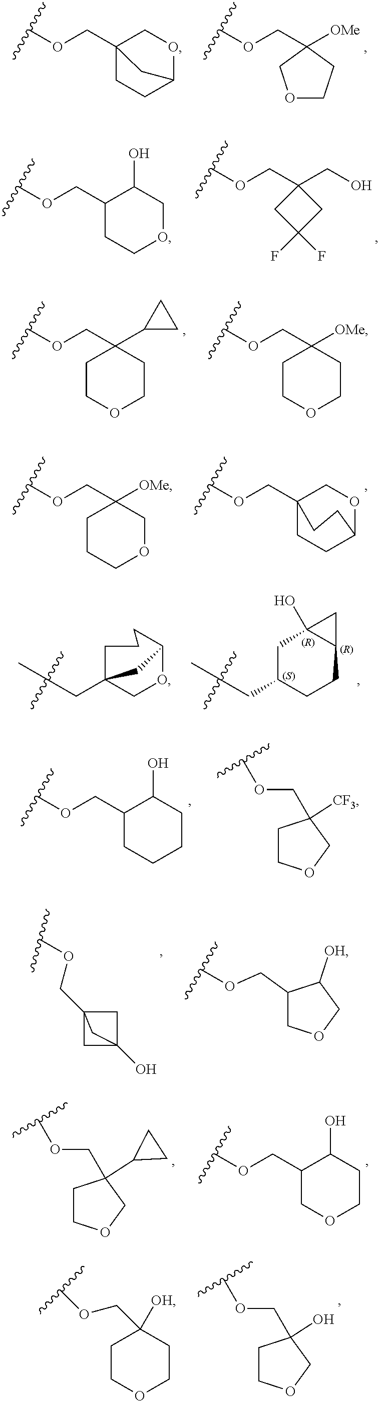

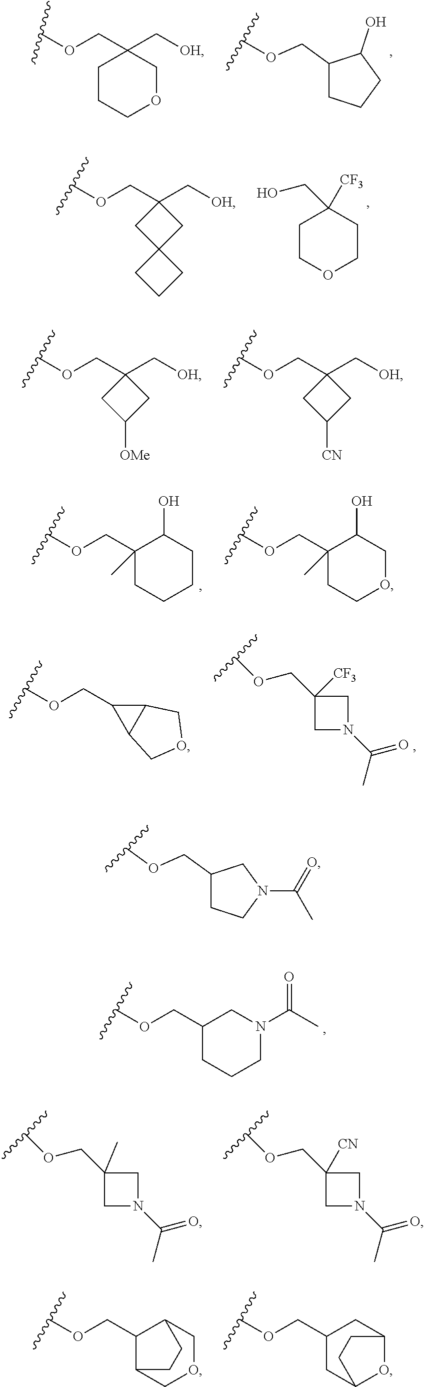

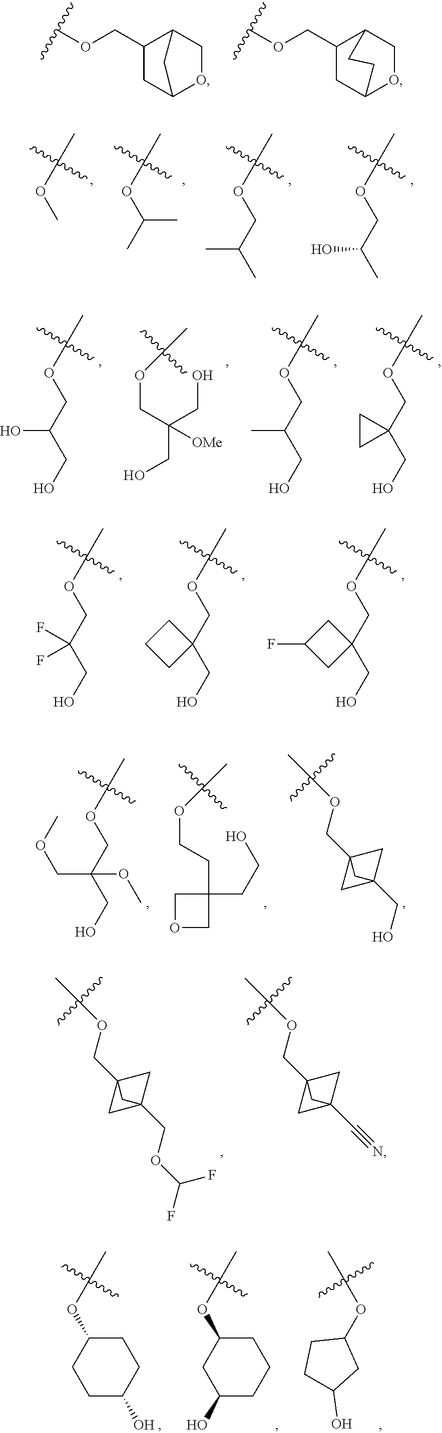

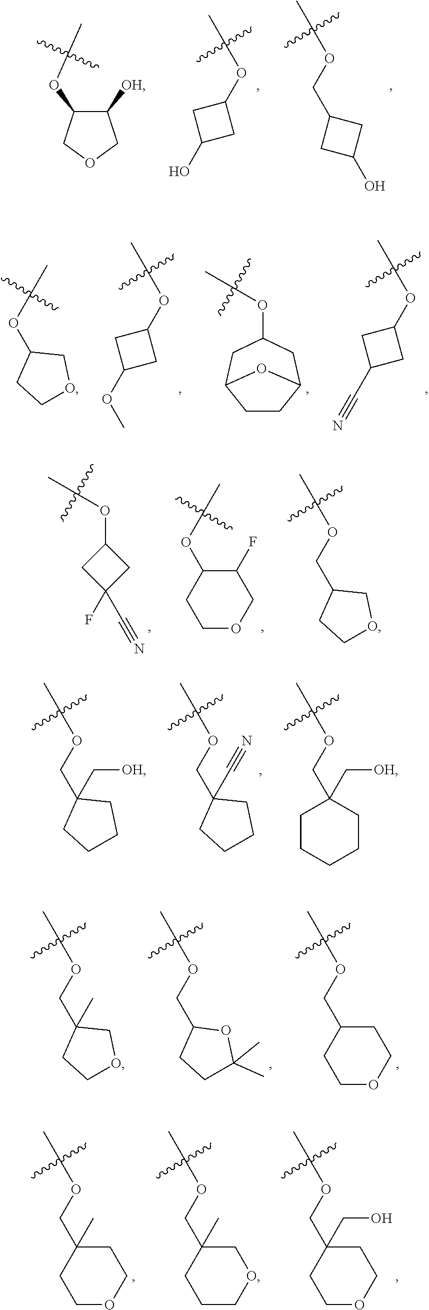

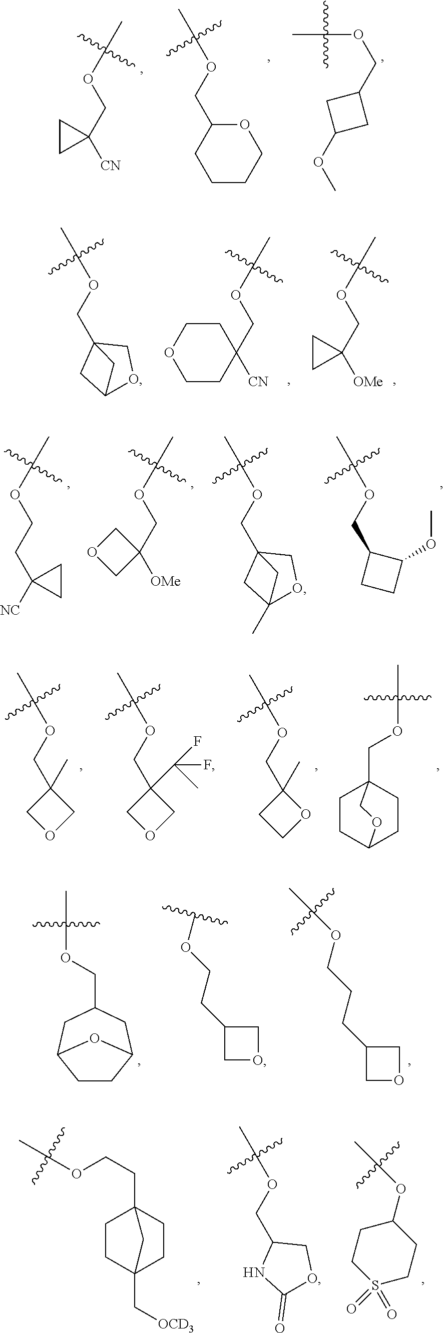

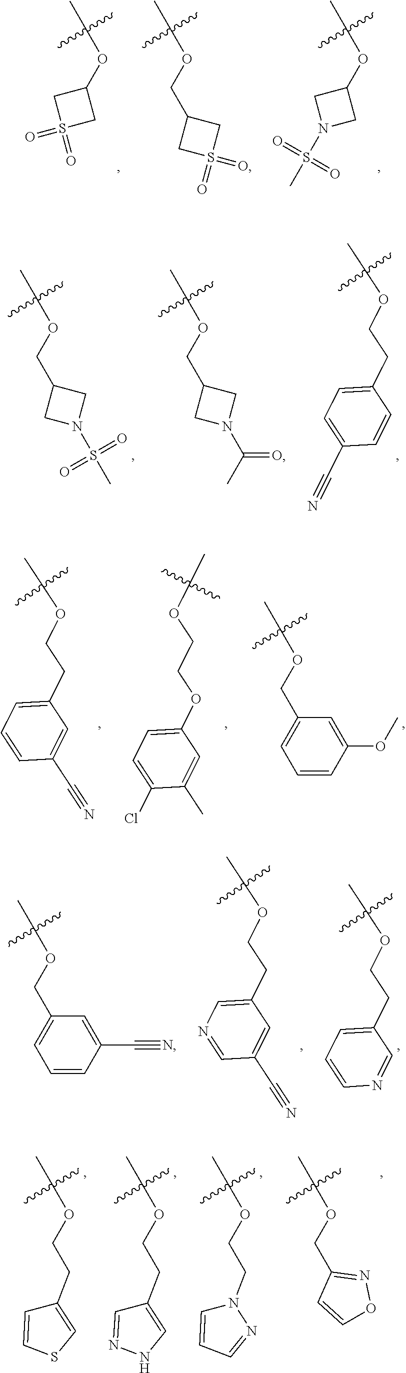

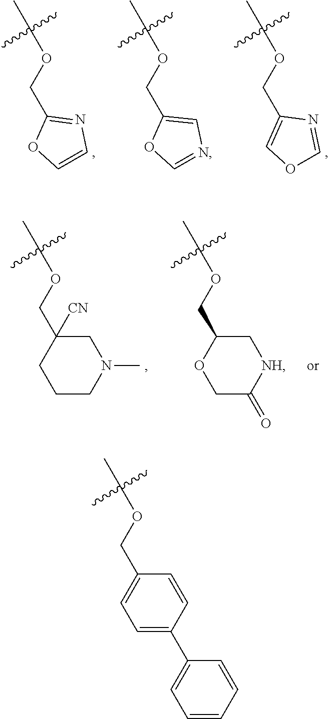 [SMILES: *OCc1ncco1 ; *OCc1cnco1 ; *OCc1cocn1 ; *OCC1(C#N)CCCN(C)C1 ; *OC[C@H]1CNC(=O)CO1 ; *OCc1ccc(-c2ccccc2)cc1]

R₁₁ is

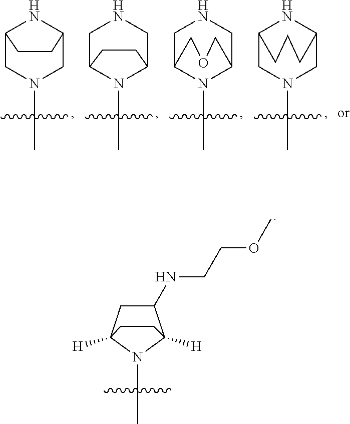

In one embodiment, A is N. In another embodiment, A is C—CN.

Aspect 42g: Provided herein are bridged compounds having formula (XIII) and pharmaceutically acceptable salts, tautomers, isotopologues, stereoisomers and prodrugs thereof,
wherein A is N or C—CN;
R₄ is F;
R₅ is —CF₃;
R₆ₐ is H;
R_{6b} is F;
R_{6d} is H;
L₁-R₈ is

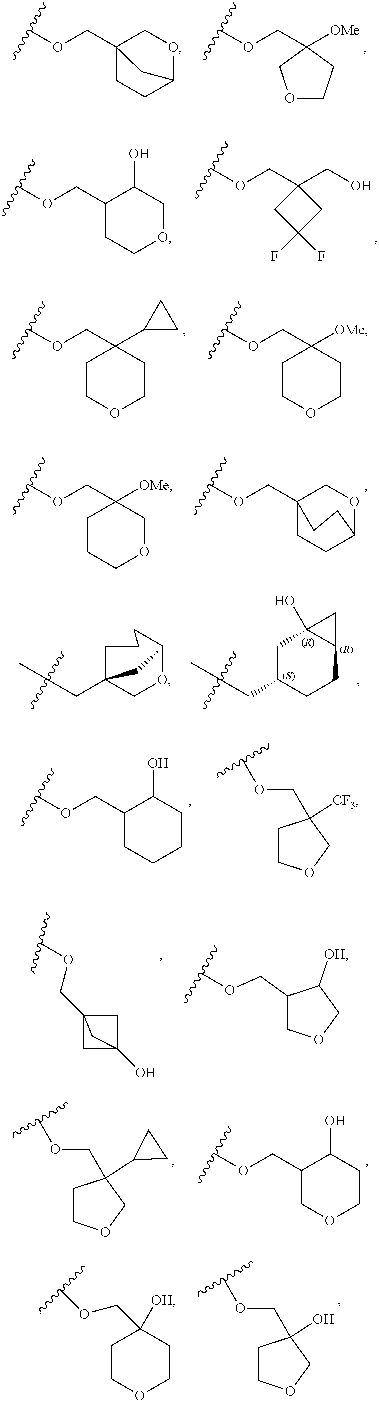

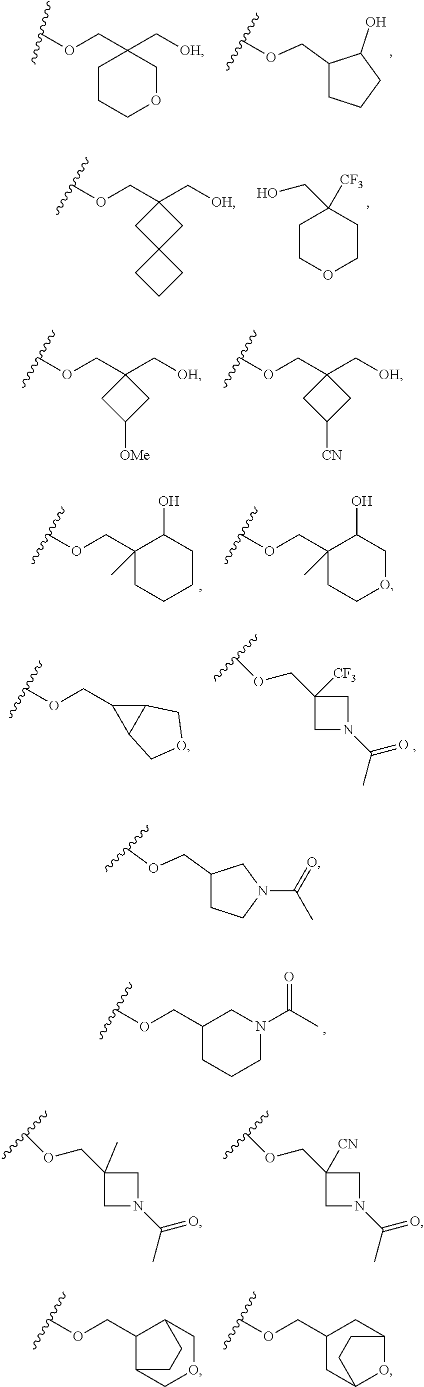

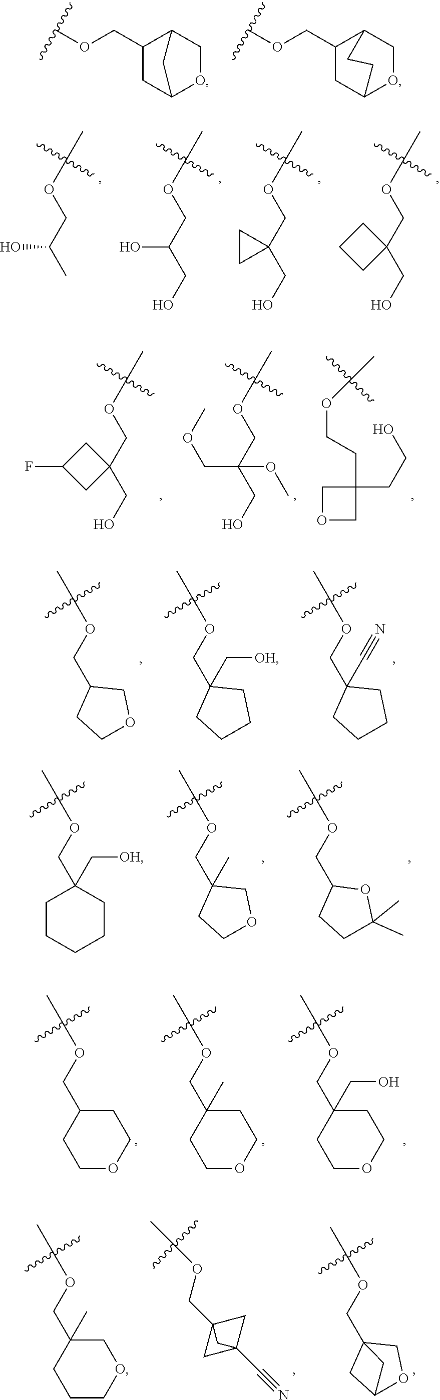

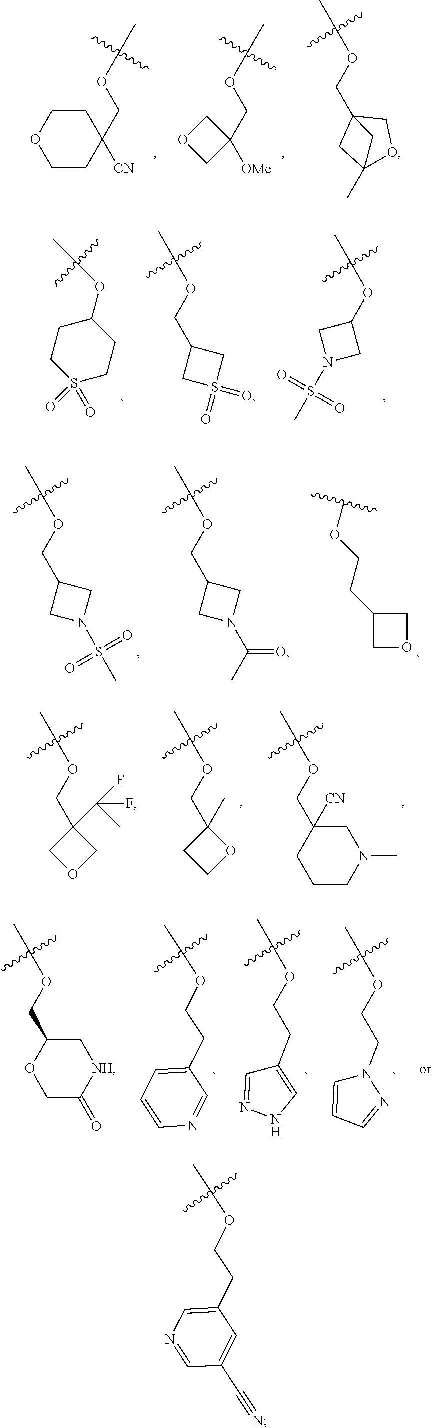

R₁₁ is

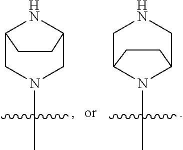 [SMILES: *N1CC2CCC(C1)N2 ; *N1C2CCC1CNC2]

In one embodiment, A is N. In another embodiment, A is C—CN.

Aspect 42h: Provided herein are bridged compounds having formula (XIII) and pharmaceutically acceptable salts, tautomers, isotopologues, stereoisomers and prodrugs thereof,
wherein A is N or C—CN;
R₄ is F;
R₅ is —CF₃;
R₆ₐ is H;
R_{6b} is F;
R_{6d} is H;
-L₁-R₈ is

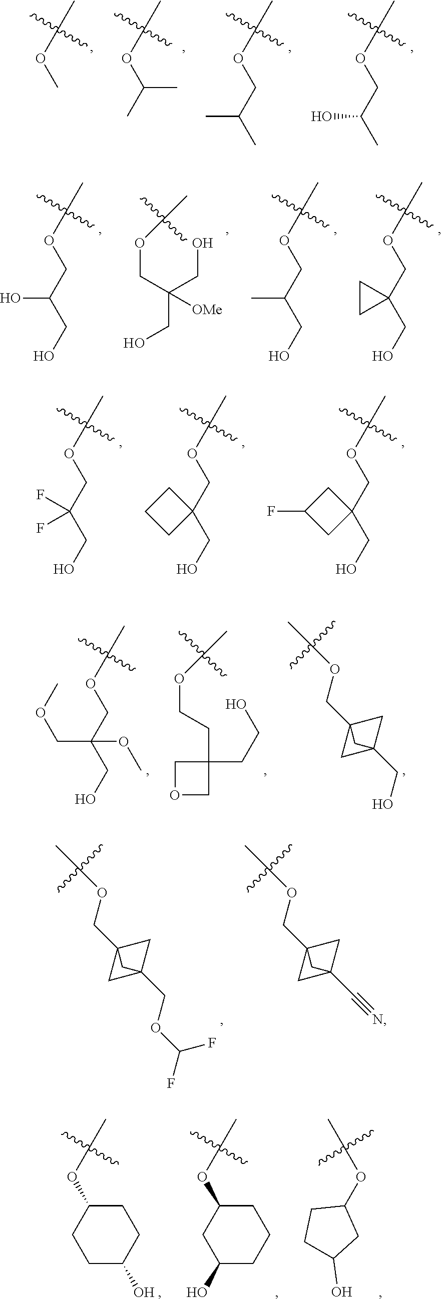

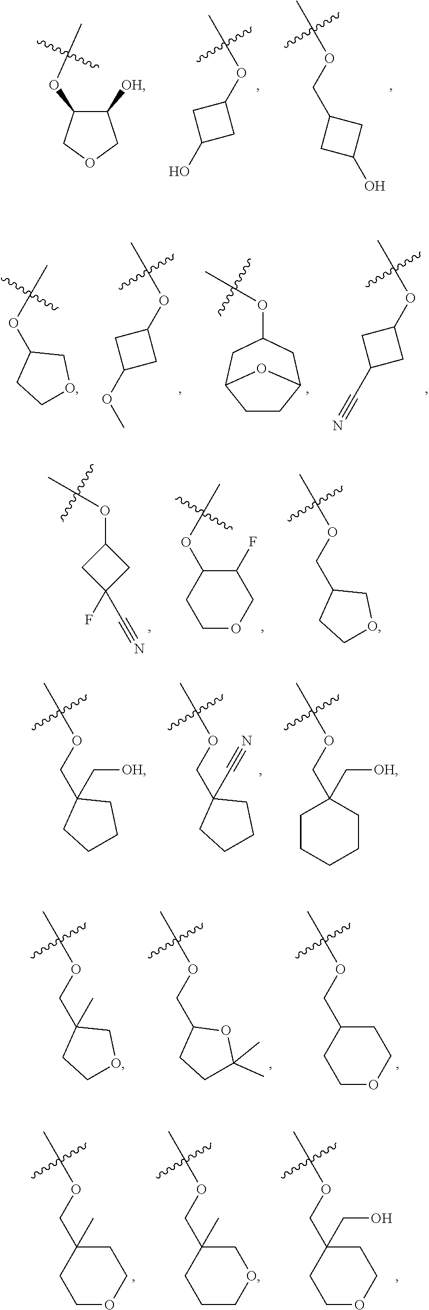

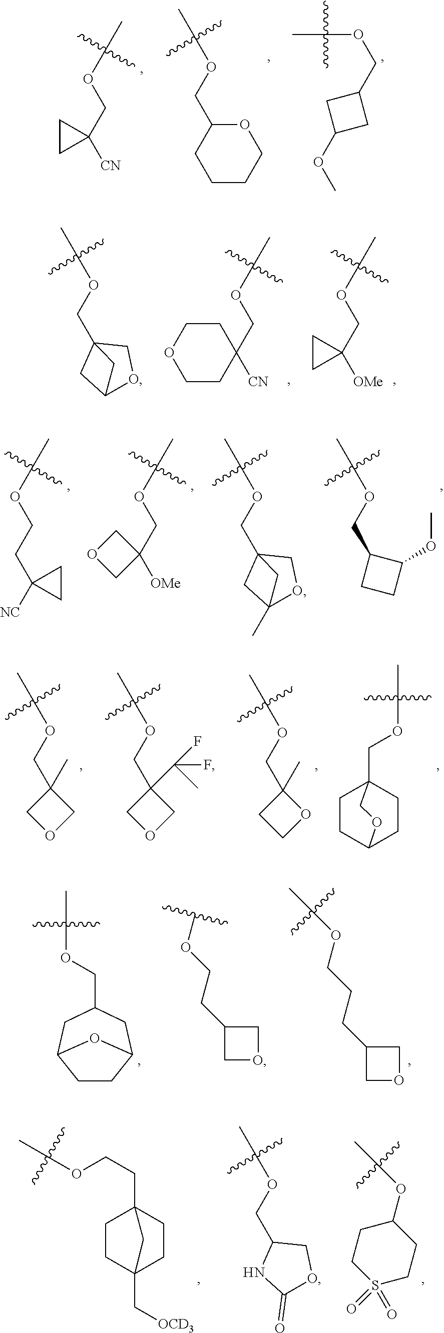

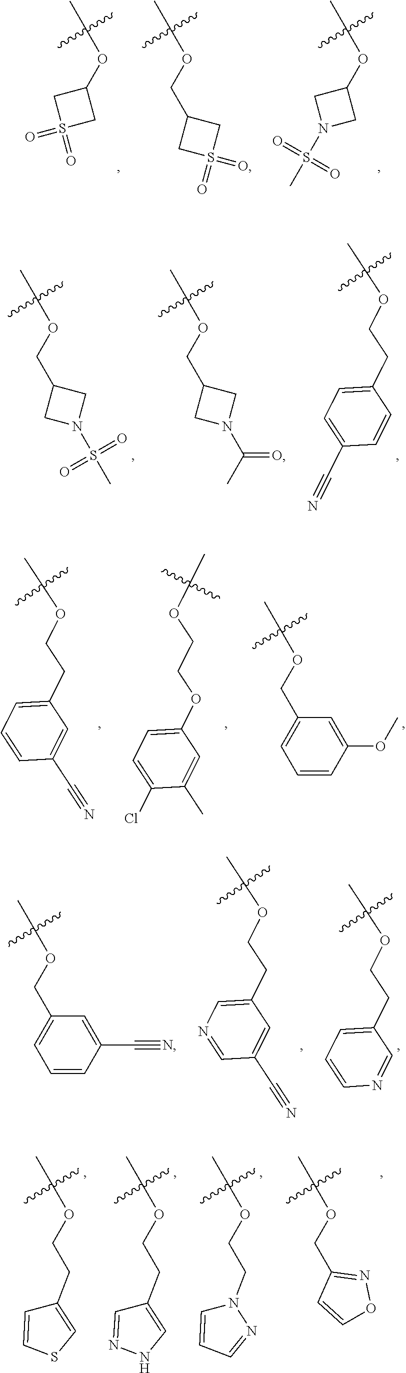

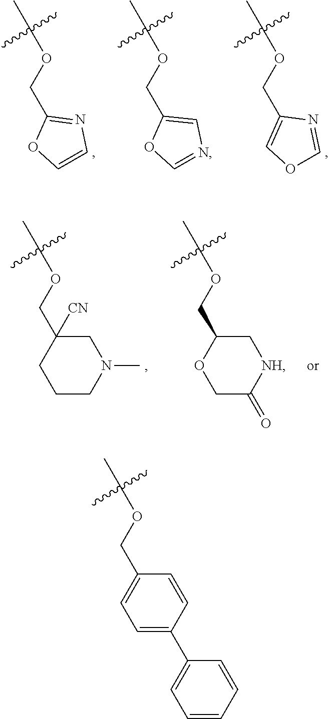 [SMILES: *OCc1ncco1 ; *OCc1cnco1 ; *OCc1cocn1 ; *OCC1(C#N)CCCN(C)C1 ; *OC[C@H]1CNC(=O)CO1 ; *OCc1ccc(-c2ccccc2)cc1]

R₁₁ is

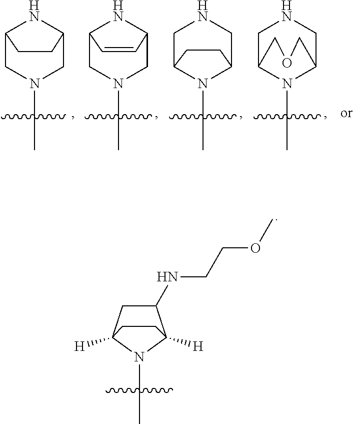

In one embodiment, A is N. In another embodiment, A is C—CN.

Aspect 42i: Provided herein are bridged compounds having formula (XIII) and pharmaceutically acceptable salts, tautomers, isotopologues, stereoisomers and prodrugs thereof,
wherein A is N or C—CN;
R₄ is F;
R₅ is —CF₃;
R₆ₐ is H;
R_{6b} is F;
R_{6d} is H;
L₁-R₈ is

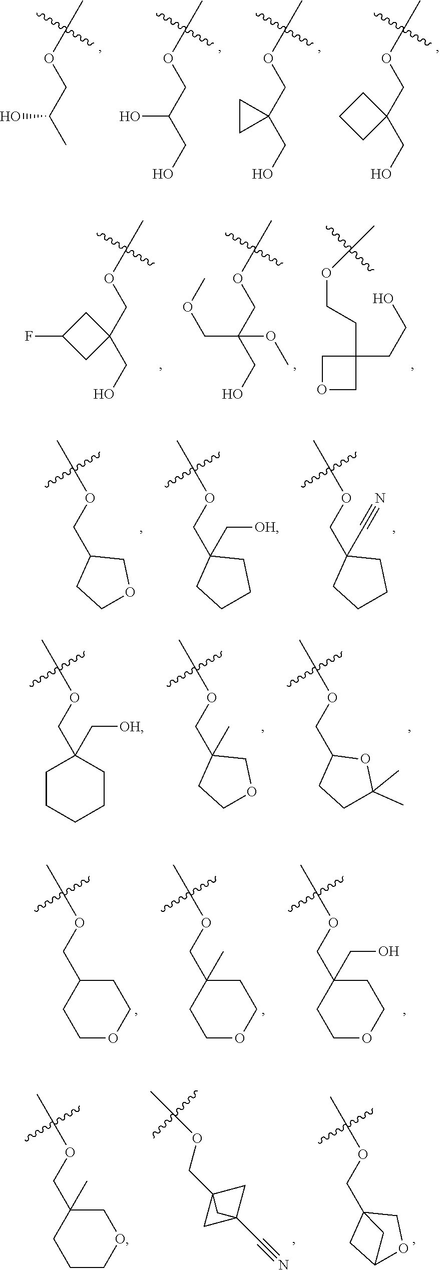

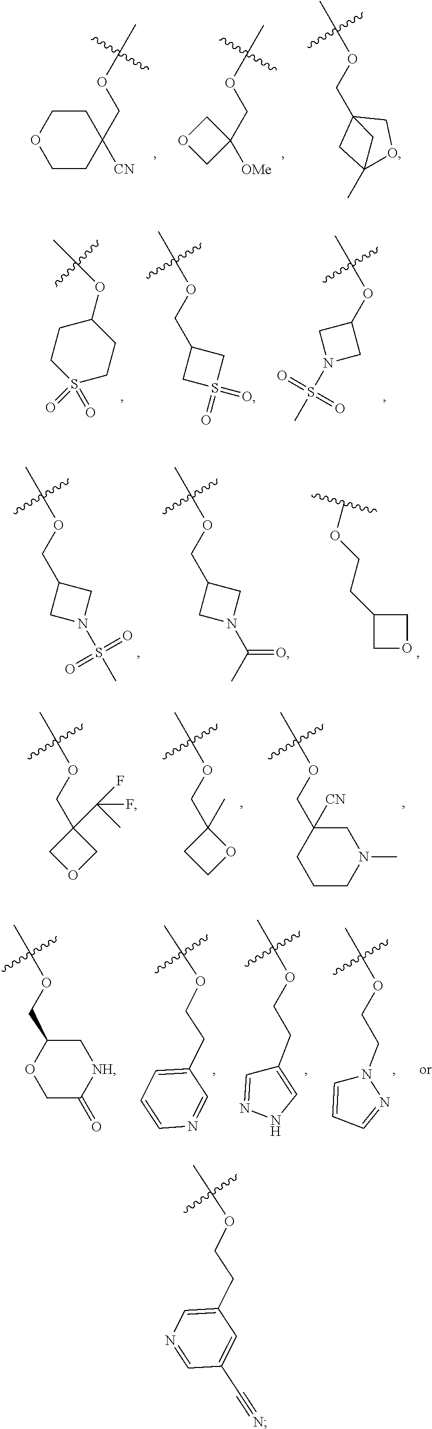

R₁₁ is

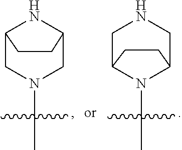 [SMILES: *N1CC2CCC(C1)N2 ; *N1C2CCC1CNC2]

In one embodiment, A is N. In another embodiment, A is C—CN.

Aspect 42j: Provided herein are bridged compounds having formula (XIII) and pharmaceutically acceptable salts, tautomers, isotopologues, stereoisomers and prodrugs thereof, wherein the bridged compound is selected from the compounds in Table 1, Table 2, and Table 6.

Aspect 42k: Provided herein are bridged compounds having formula (XIII) and pharmaceutically acceptable salts, tautomers, isotopologues, stereoisomers and prodrugs thereof, wherein the bridged compound is selected from the compounds in Table 1, Table 2, and Table 6.

Aspect 42l: In one embodiment, the bridged compound is a compound having formula (XIII) and pharmaceutically acceptable salts, tautomers, isotopologues, stereoisomers and prodrugs thereof, wherein A is N. In one embodiment, the bridged compound is a compound having formula (XIII) and pharmaceutically acceptable salts, tautomers, isotopologues, stereoisomers and prodrugs thereof, wherein A is C—CN.

Aspect 43a: Provided herein are bridged compounds having the following formula (XIV):

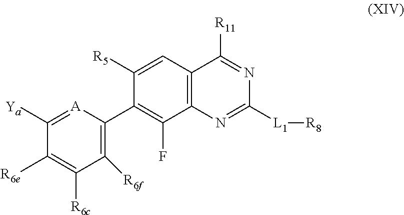

and pharmaceutically acceptable salts, tautomers, isotopologues, stereoisomers and prodrugs thereof,
wherein A is N, CH or C—CN;
Yₐ is —NH₂ or —OH;
R₅ is —CF₃, —Cl, —OCF₃, —OCF₂Cl, —SCF₃, —H, —F, —Br, —I, —CF₂CH₃, —CH₂CHF₂, —CH₂CF₃, —CN, —CHF₂, ethyl, isopropyl,

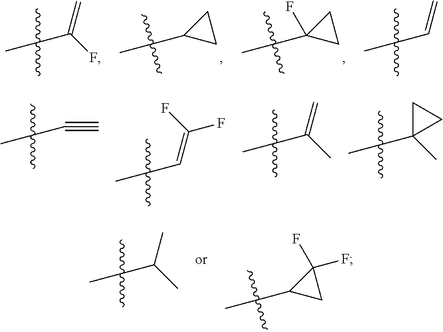

R_{6c} is H, F, Cl, methyl, ethyl, CHF₂, CN, Br,

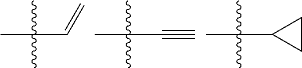

### or CF₃;

R₆ₑ is H, F, or Cl;
R_{6f} is F, Cl, methyl, —CF₃, Br, I, —CF₂CH₃, —CH₂CF₃, —OCF₃, —OCF₂Cl, —SCF₃, isopropyl, cyclopropyl, or methylcyclopropyl;
-L₁- is —O—;
R₈ is unsubstituted or substituted alkyl, unsubstituted or substituted cycloalkyl, unsubstituted or substituted cycloalkylalkyl, unsubstituted or substituted aryl, unsubstituted or substituted aralkyl, unsubstituted or substituted heterocyclyl, unsubstituted or substituted heterocyclylalkyl, unsubstituted or substituted heteroaryl, unsubstituted or substituted heteroaralkyl, unsubstituted or substituted spirocyclic ring, or unsubstituted or substituted -alkyl-spirocyclic ring,
R₁₁ is

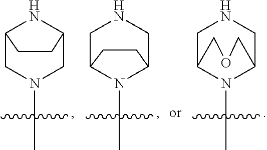

Aspect 43b: Provided herein are bridged compounds having formula (XIV) and pharmaceutically acceptable salts, tautomers, isotopologues, stereoisomers and prodrugs thereof, wherein the bridged compound is selected from the compounds in Table 1, Table 2, and Table 6.

Aspect 44a: Provided herein are bridged compounds having the following formula (XIII):

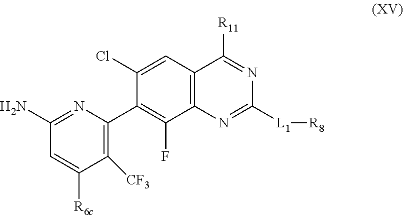

and pharmaceutically acceptable salts, tautomers, isotopologues, stereoisomers and prodrugs thereof,
wherein
R_{6c} is CH₃ or Cl;
-L₁- is —O—;
R₈ is unsubstituted or substituted alkyl, unsubstituted or substituted cycloalkyl, unsubstituted or substituted cycloalkylalkyl, unsubstituted or substituted aryl, unsubstituted or substituted aralkyl, unsubstituted or substituted heterocyclyl, unsubstituted or substituted heterocyclylalkyl, unsubstituted or substituted heteroaryl, unsubstituted or substituted heteroaralkyl, unsubstituted or substituted spirocyclic-ring, or unsubstituted or substituted -alkyl-spirocyclic ring,
R₁₁ is

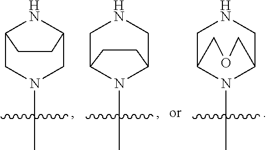

Aspect 44b: Provided herein are bridged compounds having formula (XV) and pharmaceutically acceptable salts, tautomers, isotopologues, stereoisomers and prodrugs thereof,
wherein
R_{6c} is CH₃ or Cl;
-L₁-R₈ is

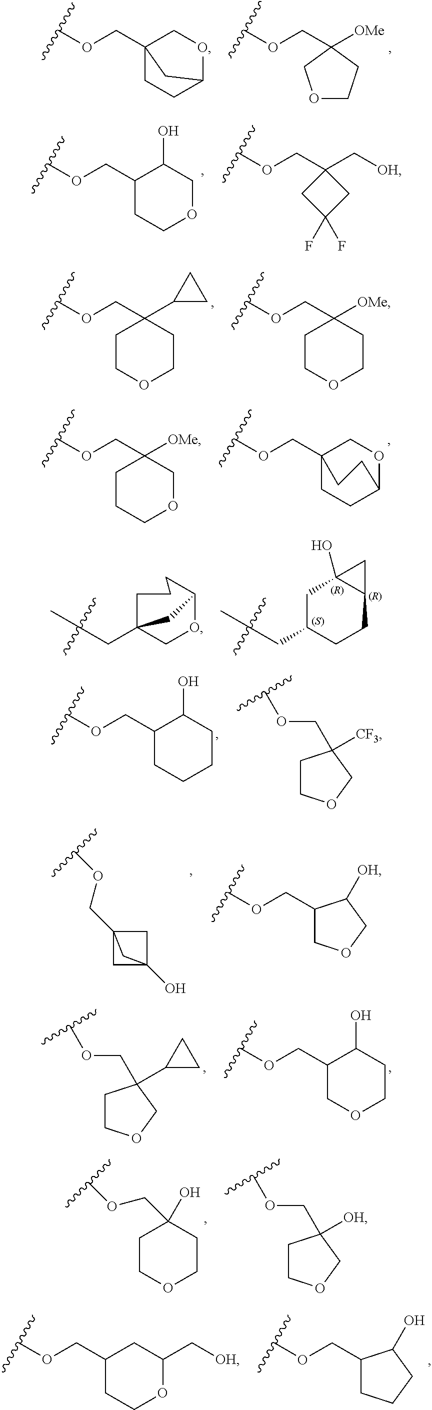

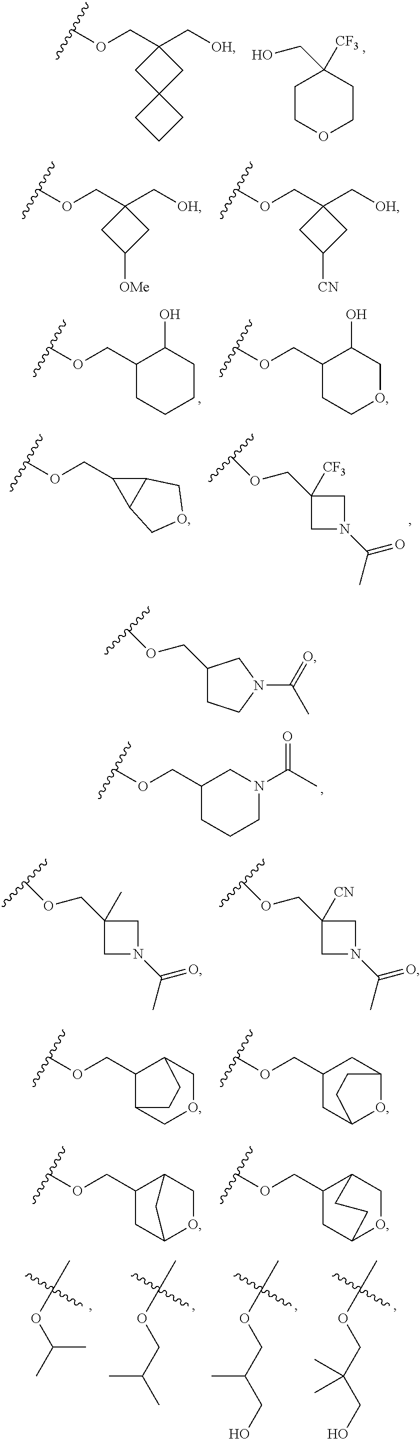

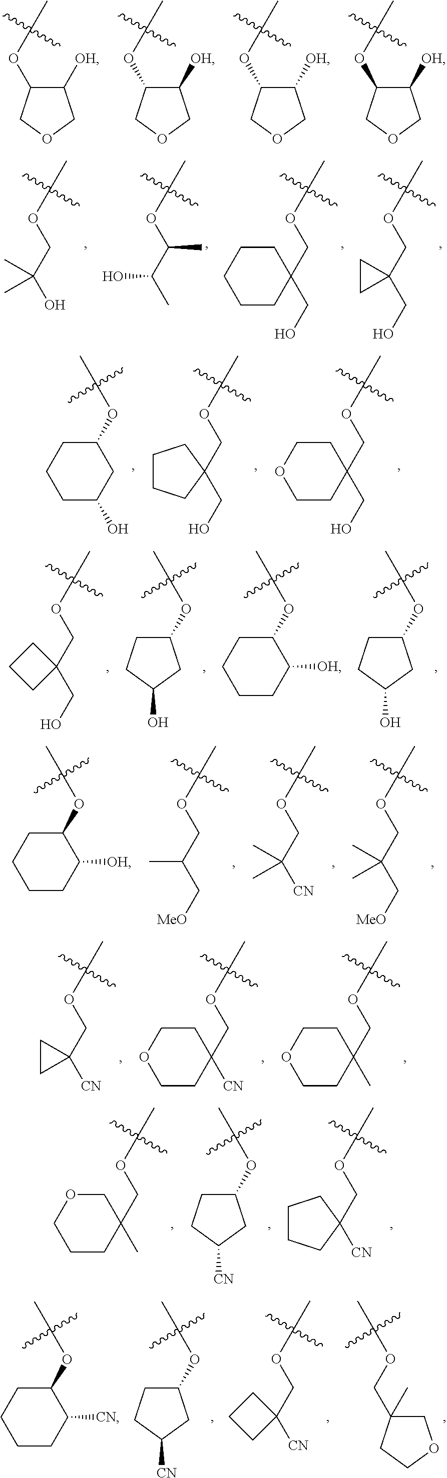

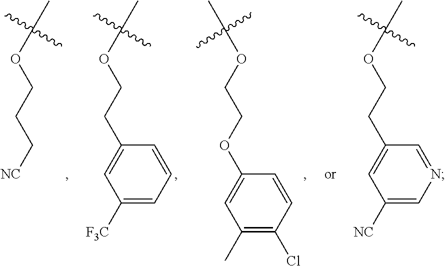 [SMILES: *OCCCC#N ; *OCCc1cccc(C(F)(F)F)c1 ; *OCCOc1ccc(Cl)c(C)c1 ; *OCCc1cncc(C#N)c1]

R₁₁ is

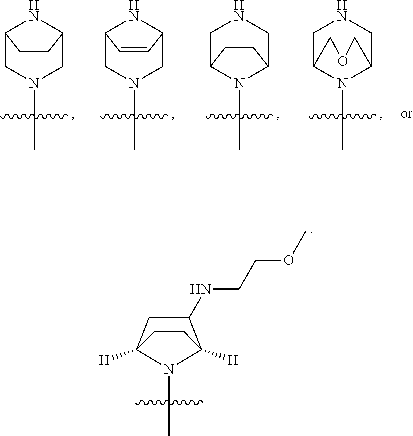

Aspect 44c: Provided herein are bridged compounds having formula (XV) and pharmaceutically acceptable salts, tautomers, isotopologues, stereoisomers and prodrugs thereof,
wherein
R_{6c} is CH₃ or Cl;
-L₁-R₈ is

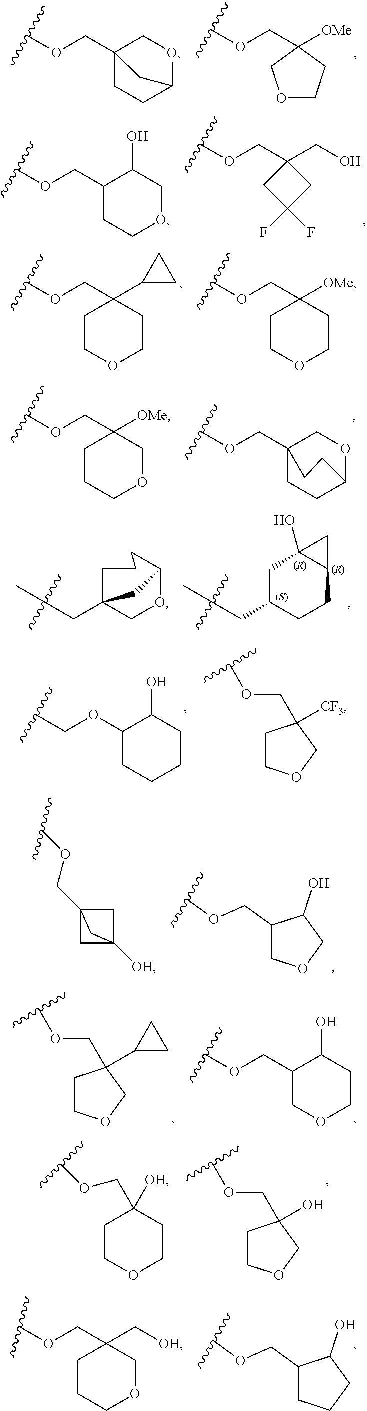

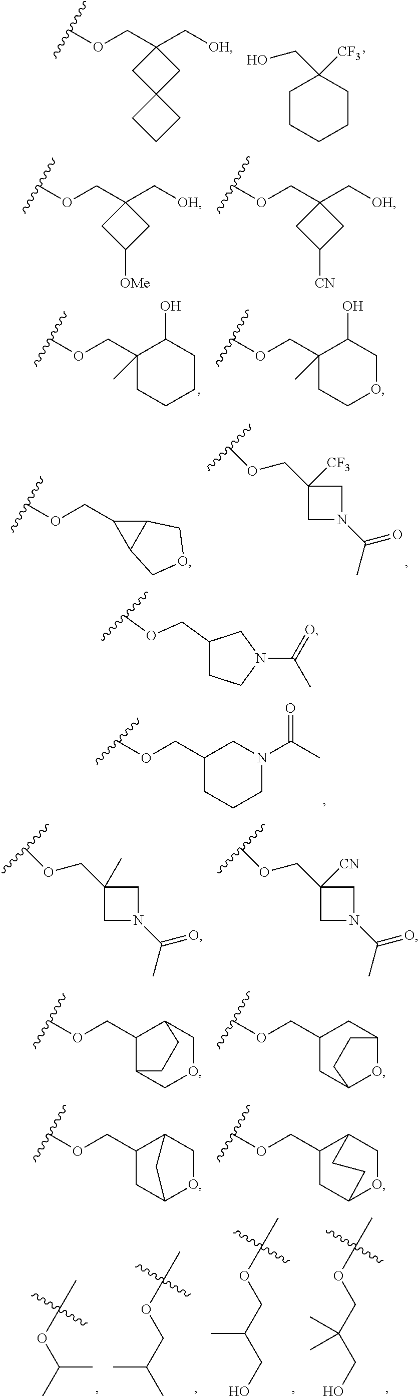

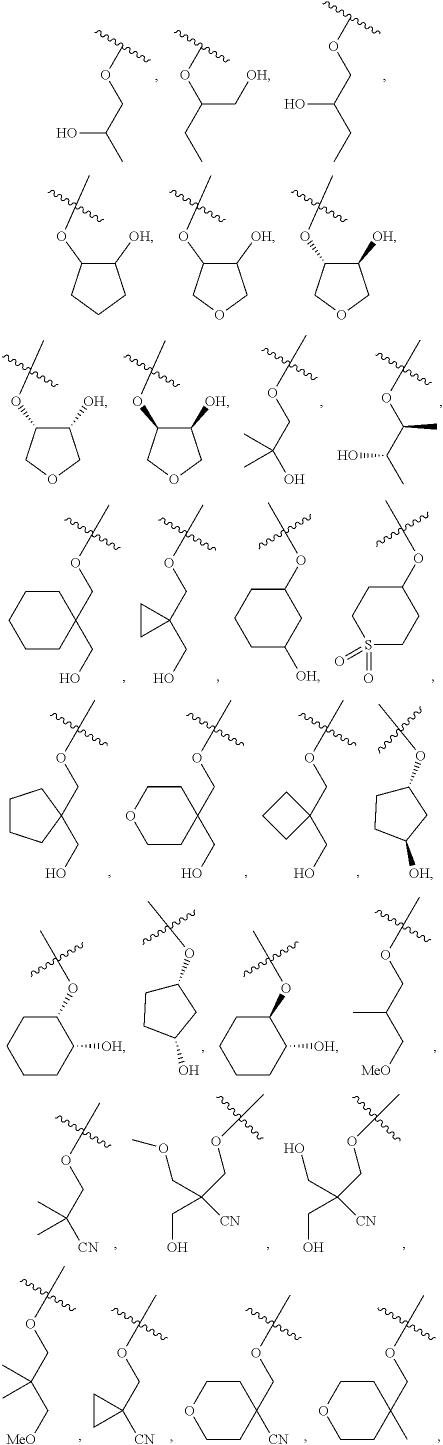

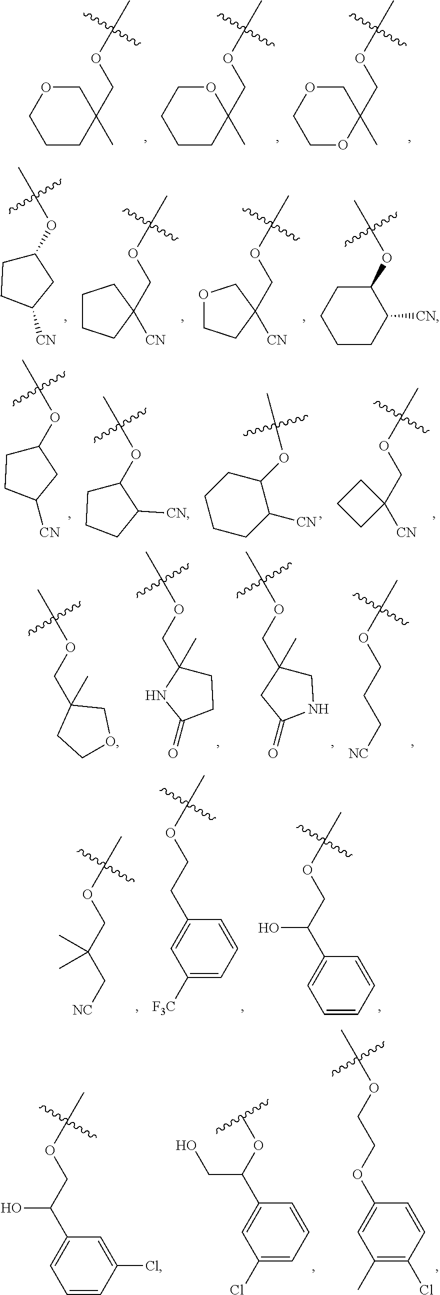

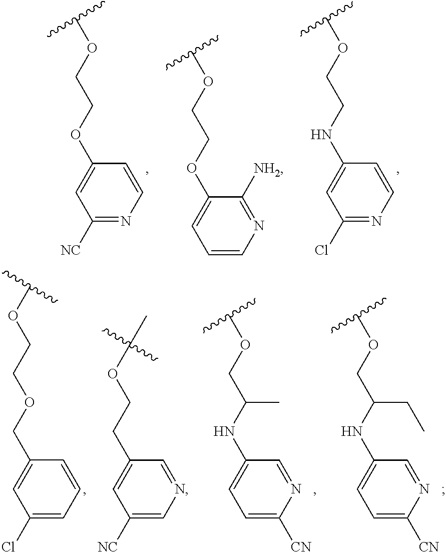

R₁₁ is

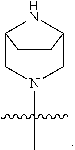 [SMILES: *N1CC2CCC(C1)N2]

Aspect 44d: Provided herein are bridged compounds having formula (XV) and pharmaceutically acceptable salts, tautomers, isotopologues, stereoisomers and prodrugs thereof,
wherein
R_{6c} is CH₃ or Cl;
-L₁-R₈ is

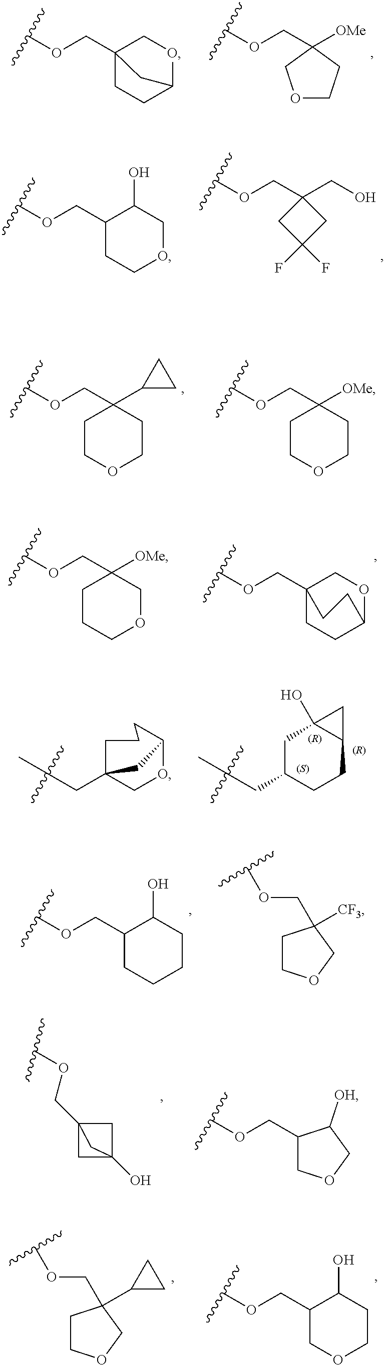

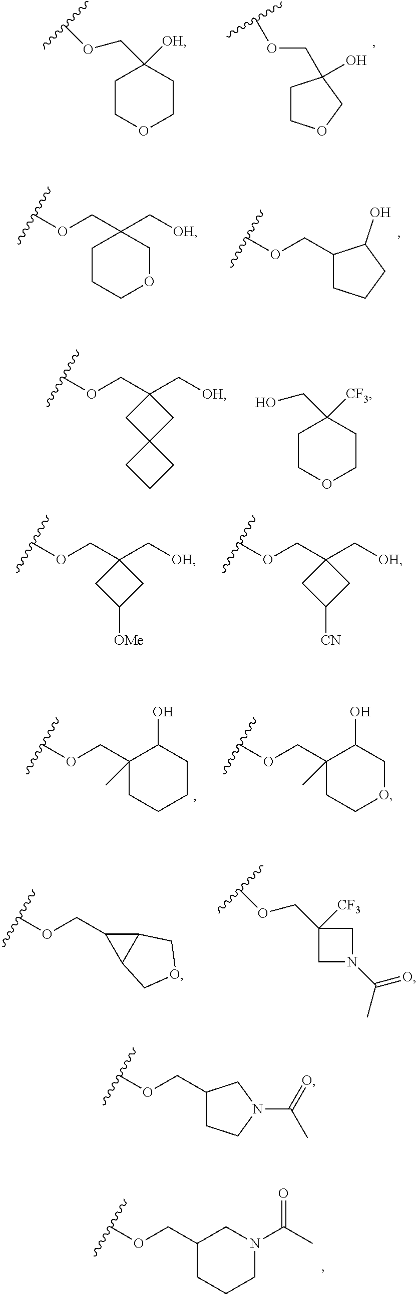

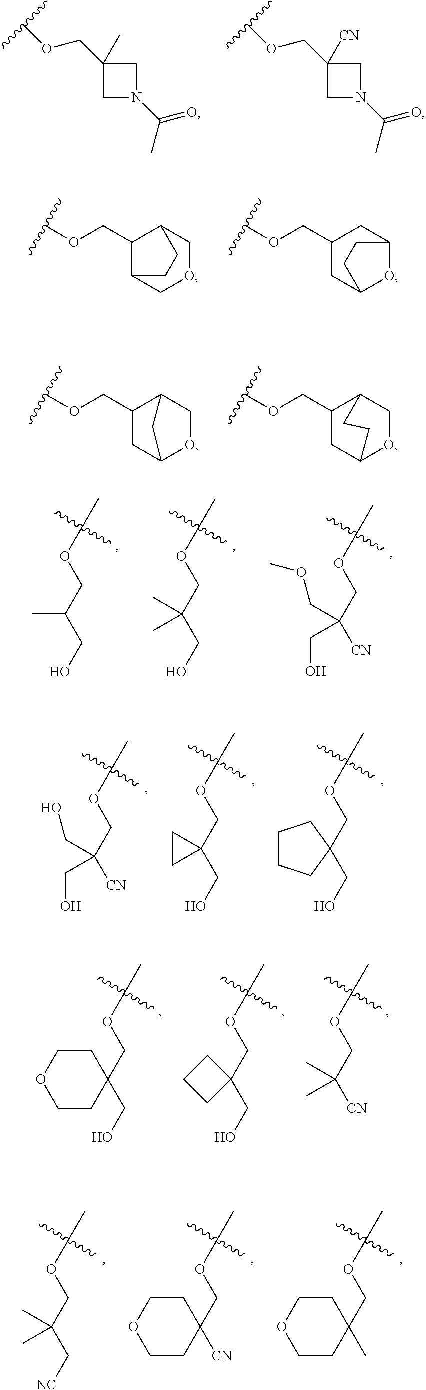

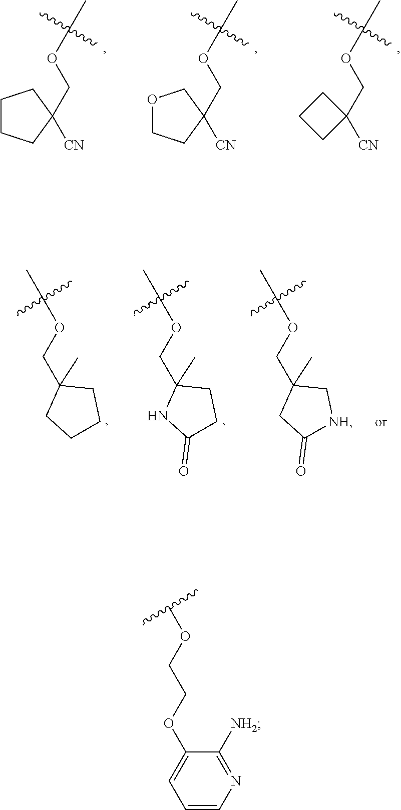

R₁₁ is

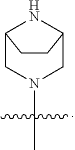 [SMILES: *N1CC2CCC(C1)N2]

Aspect 44e: Provided herein are bridged compounds having formula (XV) and pharmaceutically acceptable salts, tautomers, isotopologues, stereoisomers and prodrugs thereof,
wherein
R_{6c} is CH₃;
-L₁-R₈ is

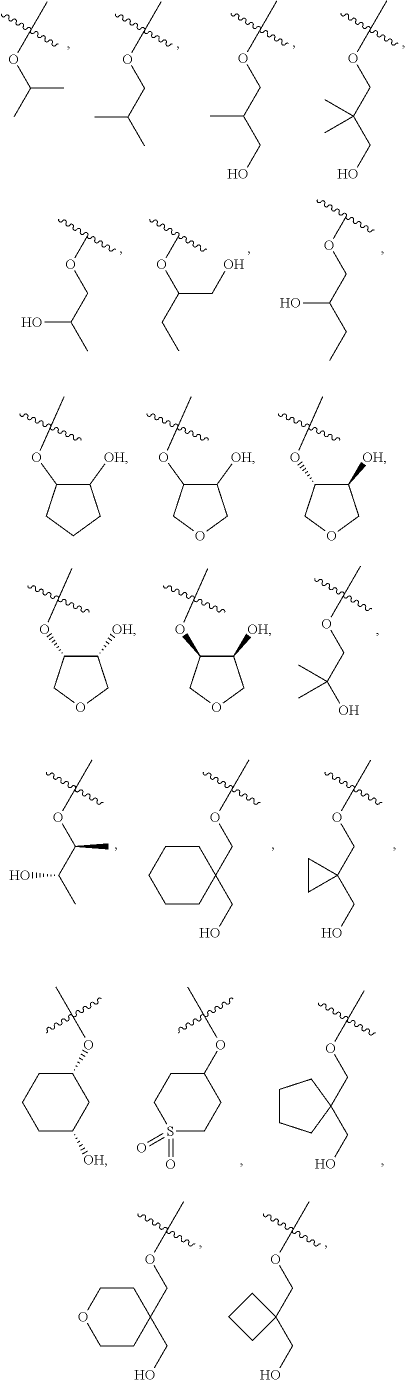

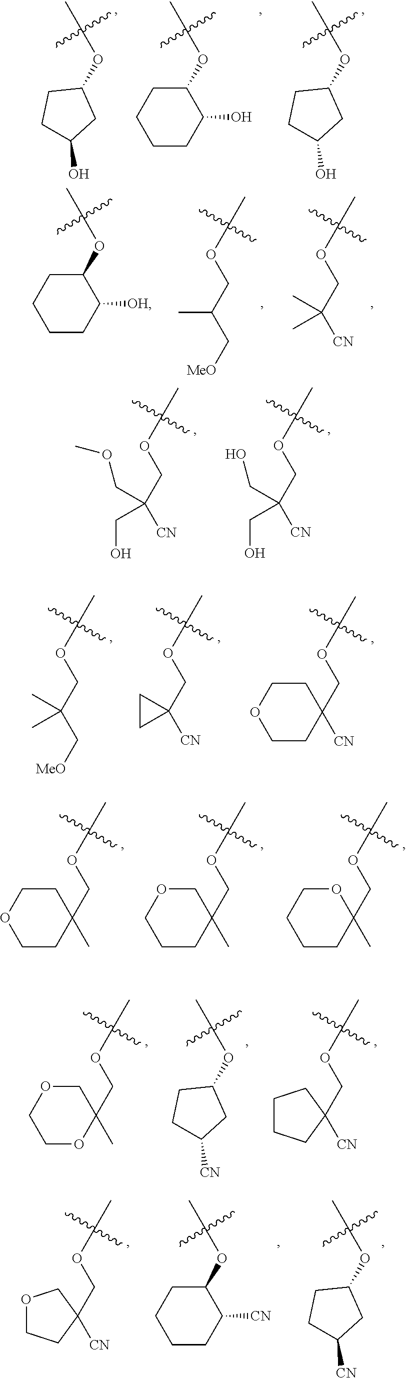

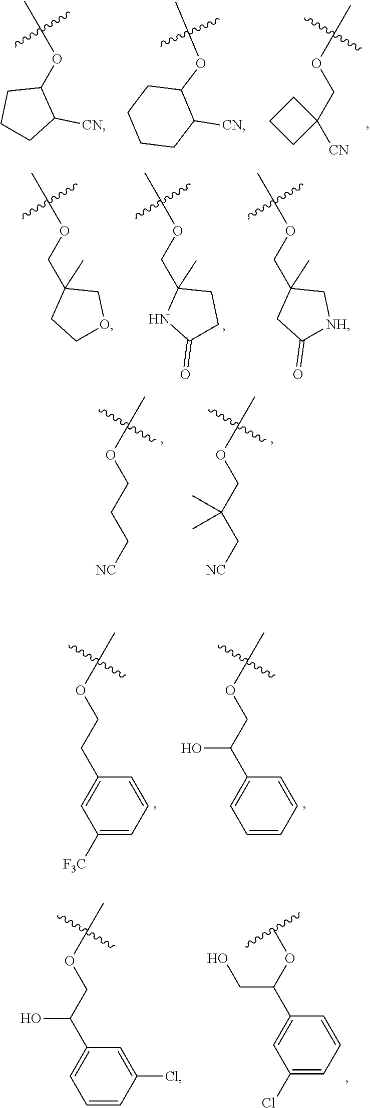

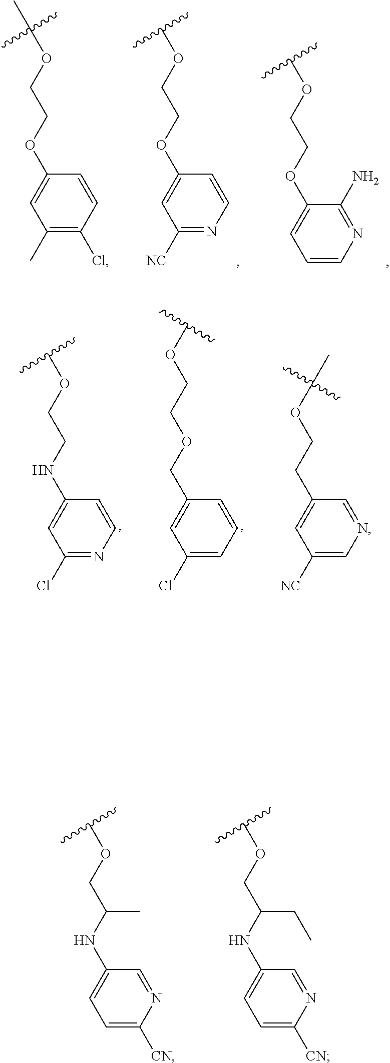

R₁₁ is

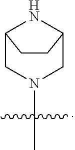 [SMILES: *N1CC2CCC(C1)N2]

Aspect 44f: Provided herein are bridged compounds having formula (XV) and pharmaceutically acceptable salts, tautomers, isotopologues, stereoisomers and prodrugs thereof,
wherein
R_{6c} is CH₃;
-L₁-R₈ is

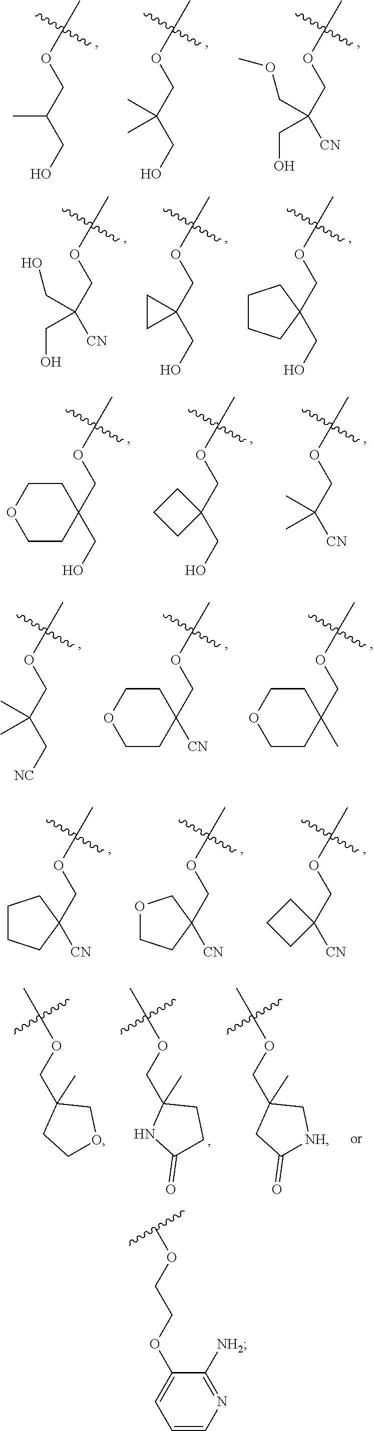

R₁₁ is

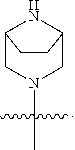 [SMILES: *N1CC2CCC(C1)N2]

Aspect 44g: Provided herein are bridged compounds having formula (XV) and pharmaceutically acceptable salts, tautomers, isotopologues, stereoisomers and prodrugs thereof, wherein the bridged compound is selected from the compounds in Table 1, Table 2, and Table 6.

Aspect 45: Provided herein are bridged compounds and pharmaceutically acceptable salts, tautomers, isotopologues, stereoisomers and prodrugs thereof, wherein the bridged compound is selected from the compounds in the tables in Table 1, Table 2, and Table 6.

Further embodiments provided herein include combinations of one or more of the particular embodiments set forth above.

### Methods for Making Bridged Compounds

The Bridged Compounds can be made using conventional organic syntheses and commercially available starting materials. By way of example and not limitation, Bridged Compounds can be prepared as outlined in Schemes A and B shown below as well as in the examples set forth herein. It should be noted that one skilled in the art would know how to modify the procedures set forth in the illustrative schemes and examples to arrive at the desired products.

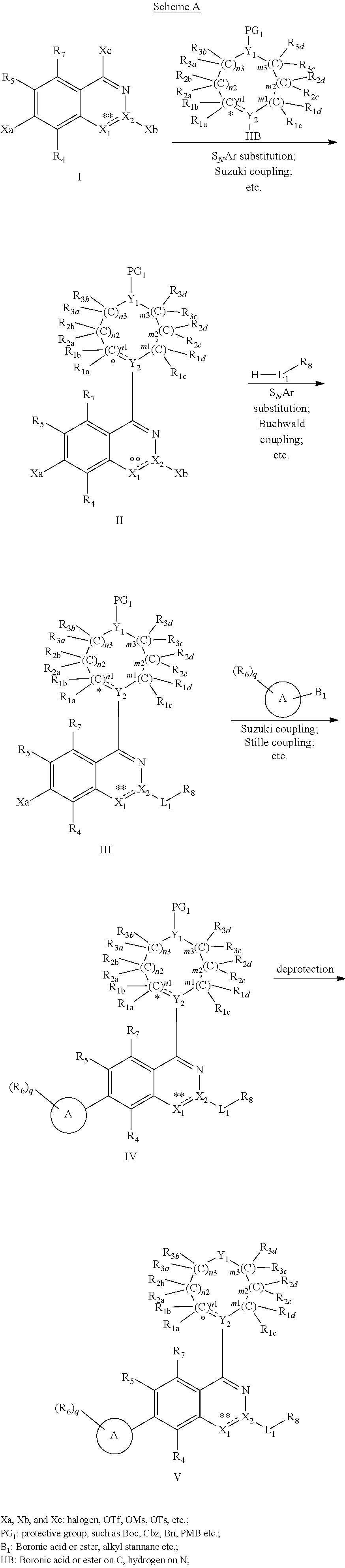

As shown in Scheme A, top piece was attached to intermediate I by S_{N}Ar substitution or Suzuki coupling etc. to give intermediate II. Then Xb was substituted by R₃-L₁-H via S_{N}Ar substitution or Buchwald coupling etc. to give intermediate III. The following Suzuki or Stille coupling etc of intermediate III gave intermediate IV, which was further deprotected to give the target compound V.

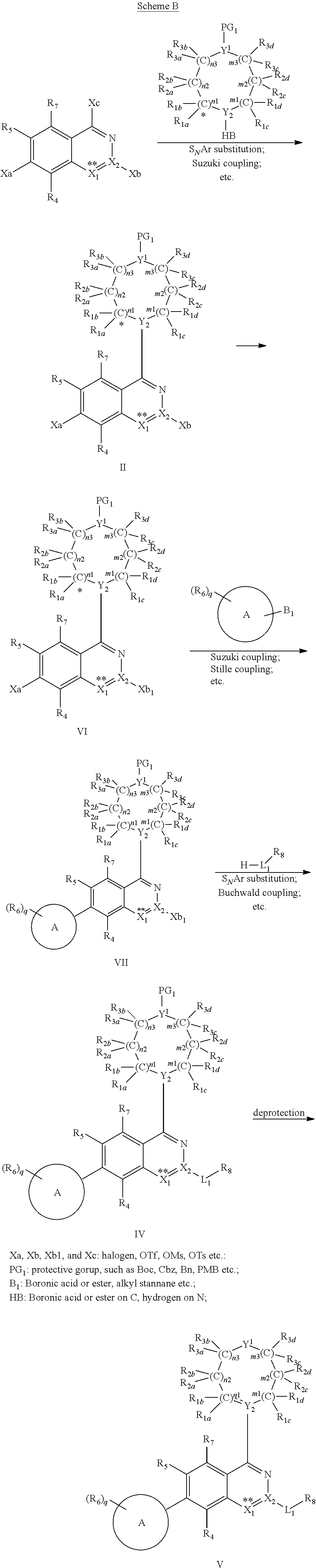

Alternatively, Bridged Compounds can be prepared as outlined in Scheme B.

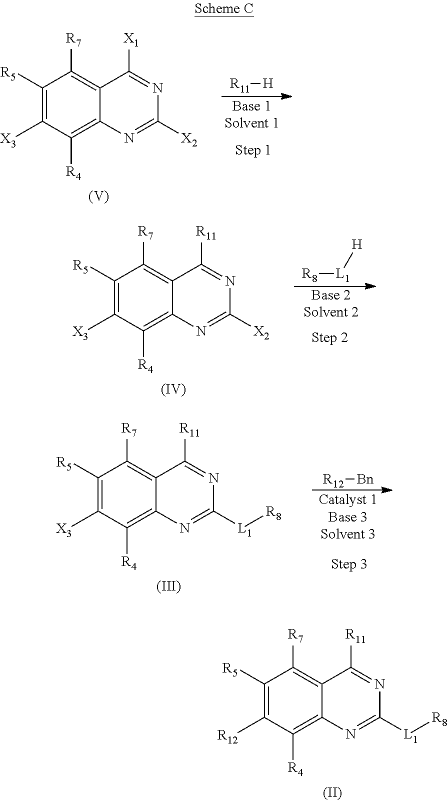

As shown in Scheme C, in certain embodiments, provided herein are methods for preparing a Bridged Compound of formula (II), comprising:
(1) contacting a compound of formula (V) with R₁₁—H in the presence of a base (Base 1, e.g., DIPEA, TEA, N-ethyl-N-isopropylpropan-2-amine, N-methylmorpholine, 1,8-diazabicyclo[5.4.0]undec-7-ene, cesium carbonate, sodium carbonate, sodium bicarbonate, potassium carbonate, or potassium phosphate) at ambient temperature (for example 0-40° C.) in an organic solvent (Solvent 1, e.g, dioxane, DMF, DMSO, NMP, or DMA), wherein X₁ is a halogen (e.g., F, Cl, Br or I) to give a compound of formula (IV);
(2) contacting a compound of formula (IV) with R₈-L₁-H in the presence of a base (Base 2, e.g., KF, DIPEA, TEA, N-methylmorpholine, 1,8-diazabicyclo[5.4.0]undec-7-ene, cesium carbonate, sodium carbonate, sodium bicarbonate, potassium carbonate, or potassium phosphate) at elevated temperature (for example 80-120° C.) in an organic solvent (Solvent 2, e.g, DMF, DMSO, NMP, or DMA), wherein X₂ is a halogen (e.g., F, Cl, Br or I) to give a compound of formula (III); and
(3) contacting a compound of formula (III) with R₁₂-Bn in the presence of a palladium catalyst (Catalyst 1, e.g., Pd(dppf)Cl₂) and a base (Base 3, e.g., DIPEA, TEA, N-methylmorpholine, 1,8-diazabicyclo[5.4.0]undec-7-ene, cesium carbonate, sodium carbonate, sodium bicarbonate, potassium carbonate, or potassium phosphate) at elevated temperature (for example 80-120° C.) in a solvent (Solvent 3, e.g, dioxane, DMF, DMSO, NMP, DMA, water or a mixture thereof), wherein X₃ is a halogen (e.g., F, Cl, Br or I) and Bn is an organoborane (e.g., —B(OH)₂ and bis(pinacolato)diboron) or organotin (e.g., Sn(Bu)₃) to give a compound of formula (II),
wherein R₄, R₅, R₇, R₈, R₁₁, R₁₂ and L₁ are as defined herein.

Provided herein are methods of preparing a Bridged Compound of formula (II), the method comprising contacting a compound of formula (III) with R₁₂-Bn in the presence of a palladium catalyst (Catalyst 1, e.g., Pd(dppf)Cl₂) and a base (Base 3, e.g., DIPEA, TEA, N-methylmorpholine, 1,8-diazabicyclo[5.4.0]undec-7-ene, cesium carbonate, sodium carbonate, sodium bicarbonate, potassium carbonate, or potassium phosphate) at elevated temperature (for example 80-120° C.) in a solvent (Solvent 3, e.g., dioxane, DMF, DMSO, NMP, DMA, water or a mixture thereof), wherein X₃ is a halogen (e.g., F, Cl, Br or I) and Bn is an organoborane (e.g., —B(OH)₂ and bis(pinacolato)diboron) or organotin (e.g., Sn(Bu)₃).

Provided herein are methods of preparing a compound of formula (III), the method comprising contacting a compound of formula (IV) with R₈-L₁-H in the presence of a base (Base 2, e.g., KF, DIPEA, TEA, N-methylmorpholine, 1,8-diazabicyclo[5.4.0]undec-7-ene, cesium carbonate, sodium carbonate, sodium bicarbonate, potassium carbonate, or potassium phosphate) at elevated temperature (for example 80-120° C.) in an organic solvent (Solvent 2, e.g, DMF, DMSO, NMP, or DMA), wherein X₂ is a halogen (e.g., F, Cl, Br or I).

Provided herein are methods of preparing a compound of formula (IV), the method comprising contacting a compound of formula (V) with R₁₁—H in the presence of a base (Base 1, e.g., dioxane, DIPEA, TEA, N-methylmorpholine, 1,8-diazabicyclo[5.4.0]undec-7-ene, cesium carbonate, sodium carbonate, sodium bicarbonate, potassium carbonate, or potassium phosphate) at ambient temperature (for example 0-40° C.) in an organic solvent (Solvent 1, e.g, DMF, DMSO, NMP, or DMA), wherein X₁ is a halogen (e.g., F, Cl, Br or I).

As shown in Scheme C, in certain embodiments, provided herein are methods for preparing 4-(4-(3-oxa-7,9-diazabicyclo[3.3.1]nonan-9-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine, comprising:
(1) contacting 7-bromo-2,4-dichloro-8-fluoro-6-(trifluoromethyl)quinazoline with tert-butyl 3-oxa-7,9-diazabicyclo[3.3.1]nonane-7-carboxylate in the presence of TEA at room temperature in CH₂Cl₂;
(2) contacting tert-butyl 9-(7-bromo-2,8-difluoro-6-(trifluoromethyl)quinazolin-4-yl)-3-oxa-7,9-diazabicyclo[3.3.1]nonane-7-carboxylate with ((2R,7aS)-2-fluorotetrahydro-1H-pyrrolizin-7a(5H)-yl)methanol in the presence of NaH at room temperature in THF; and
(3) contacting tert-butyl 9-(7-bromo-8-fluoro-2-(((2R,7aS)-2-fluorotetrahydro-1H-pyrrolizin-7a(5H)-yl)methoxy)-6-(trifluoromethyl)quinazolin-4-yl)-3-oxa-7,9-diazabicyclo[3.3.1]nonane-7-carboxylate with (2-amino-7-fluorobenzo[d]thiazol-4-yl)boronic acid in the presence of Pd(dtbpf)Cl₂ and K₃PO₄ at elevated temperature (for example 80-120° C.) in a mixture of dioxane and H₂O.

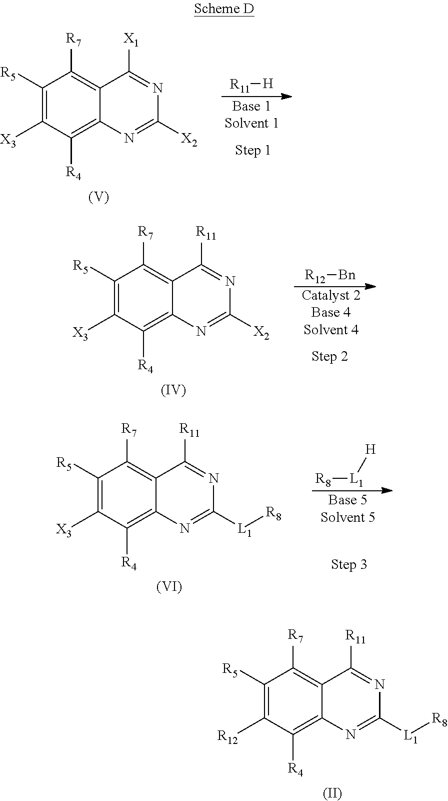

As shown in Scheme D, in certain embodiments, provided herein are methods for preparing a Bridged Compound of formula (II), comprising:
(1) contacting a compound of formula (V) with R₁₁—H in the presence of a base (Base 1, e.g., dioxane, DIPEA, TEA, N-methylmorpholine, 1,8-diazabicyclo[5.4.0]undec-7-ene, cesium carbonate, sodium carbonate, sodium bicarbonate, potassium carbonate, or potassium phosphate) at ambient temperature (for example 0-40° C.) in an organic solvent (Solvent 1, e.g, DMF, DMSO, NMP, or DMA), wherein X₁ is a halogen (e.g., F, Cl, Br or I) to give a compound of formula (IV);
(2) contacting a compound of formula (IV) with R₁₂-Bn in the presence of a palladium catalyst (Catalyst 2, e.g., Pd(PPh₃)₄ and/or CuI; or Pd(dppf)Cl₂) and a base (Base 4, e.g., LiCl, DIPEA, TEA, N-methylmorpholine, 1,8-diazabicyclo[5.4.0]undec-7-ene, cesium carbonate, sodium carbonate, sodium bicarbonate, potassium carbonate, or potassium phosphate) at elevated temperature (for example 80-120° C.) in a solvent (Solvent 4, e.g, dioxane, DMF, DMSO, NMP, DMA, water or a mixture thereof), wherein X₃ is a halogen (e.g., F, Cl, Br or I) and Bn is an organoborane (e.g., —B(OH)₂ and bis(pinacolato)diboron) or organotin (e.g., Sn(Bu)₃) to give a compound of formula (VI),
(3) contacting a compound of formula (VI) with R₈-L₁-H in the presence of a base (Base 5, e.g., NaH, KF, DIPEA, TEA, N-methylmorpholine, 1,8-diazabicyclo[5.4.0]undec-7-ene, cesium carbonate, sodium carbonate, sodium bicarbonate, potassium carbonate, or potassium phosphate) at ambient temperature (for example 0-40° C.) in an organic solvent (Solvent 5, e.g, THF, DMF, DMSO, NMP, or DMA), wherein X₂ is a halogen (e.g., F, Cl, Br or I) to give a compound of formula (II); and
wherein R₄, R₅, R₇, R₈, R₁₁, R₁₂ and L₁ are as defined herein.

Provided herein are methods of preparing a Bridged Compound of formula (II), the method comprising contacting a compound of formula (VI) with R₈-L₁-H in the presence of a base (Base 5, e.g., NaH, KF, DIPEA, TEA, N-methylmorpholine, 1,8-diazabicyclo[5.4.0]undec-7-ene, cesium carbonate, sodium carbonate, sodium bicarbonate, potassium carbonate, or potassium phosphate) at ambient temperature (for example 0-40° C.) in an organic solvent (Solvent 5, e.g, THF, DMF, DMSO, NMP, or DMA), wherein X₃ is a halogen (e.g., F, Cl, Br or I) and Bn is an organoborane (e.g., —B(OH)₂ and bis(pinacolato)diboron).

Provided herein are methods of preparing a compound of formula (VI), the method comprising contacting a compound of formula (IV) with R₁₂-Bn in the presence of a palladium catalyst (Catalyst 2, e.g., Pd(PPh₃)₄ and/or CuI; or Pd(dppf)Cl₂) and a base (Base 4, e.g., LiCl, DIPEA, TEA, N-methylmorpholine, 1,8-diazabicyclo[5.4.0]undec-7-ene, cesium carbonate, sodium carbonate, sodium bicarbonate, potassium carbonate, or potassium phosphate) at elevated temperature (for example 80-120° C.) in a solvent (Solvent 4, e.g, dioxane, DMF, DMSO, NMP, DMA, water or a mixture thereof), wherein X₃ is a halogen (e.g., F, Cl, Br or I) and Bn is an organoborane (e.g., —B(OH)₂ and bis(pinacolato)diboron) or organotin (e.g., Sn(Bu)₃) to give a compound of formula (VI).

Provided herein are methods of preparing a compound of formula (IV), the method comprising contacting a compound of formula (V) with R₁₁—H in the presence of a base (Base 1, e.g., dioxane, DIPEA, TEA, N-methylmorpholine, 1,8-diazabicyclo[5.4.0]undec-7-ene, cesium carbonate, sodium carbonate, sodium bicarbonate, potassium carbonate, or potassium phosphate) at ambient temperature (for example 0-40° C.) in an organic solvent (Solvent 1, e.g, DMF, DMSO, NMP, or DMA), wherein X₁ is a halogen (e.g., F, Cl, Br or I).

In certain embodiments, the palladium catalyst (Catalyst 1) is chloro(2-dicyclohexylphosphino-2′,6′-di-i-propoxy-1,1′-biphenyl)[2-(2-aminoethylphenyl)]palladium(II), chloro(2-dicyclohexylphosphino-2′,6′-di-i-propoxy-1,1′-biphenyl)(2-amino-1,1′-biphenyl-2-yl)palladium(II), chloro(2-dicyclohexylphosphino-2′,6′-dimethoxy-1,1′-biphenyl)[2-(2-aminoethylphenyl)]palladium(II) dichloromethane adduct, chloro(2-dicyclohexylphosphino-2′,6′-dimethoxy-1,1′-biphenyl)(2′-amino-1,1′-biphenyl-2-yl) palladium(II), chloro(2-dicyclohexylphosphino-2′,4′,6′-tri-i-propyl-1,1′-biphenyl)[2-(2-aminoethyl)phenyl]palladium(II), chloro(2-dicyclohexylphosphino-2′,4′,6′-triisopropyl-1,1′-biphenyl)[2-(2′-amino-1,1′-biphenyl)]palladium(II), chloro[2-(dicyclohexylphosphino)-3,6-dimethoxy-2′-4′-6′-tri-i-propyl-1,1′-biphenyl][2-(2-aminoethyl)phenyl]palladium(II), [1,1′-bis(di-tert-butylphosphino)ferrocene]dichloropalladium, [1,1′-bis(diphenylphosphino)ferrocene]dichloropalladium, bis(triphenylphosphine)palladium(II) chloride complex, tetrakis(triphenylphosphine)palladium(0), dichlorobis(tricyclohexylphoshine)palladium(II), palladium(II) chloride with 2′-(dicyclohexylphosphino)acetophenone ethylene ketal ligand, palladium (II) chloride with 1,2,3,4,5-pentaphenyl-1′-(di-tert-butylphosphino)ferrocene ligand, palladium (II) chloride with 2-dicyclohexylphosphino-2′-(N,N-dimethylamino)biphenyl ligand, palladium (II) chloride with 4,5-bis(diphenylphosphino)-9,9-dimethylxanthene ligand or palladium (II) chloride with 2-dicyclohexylphosphino-2′-methylbiphenyl ligand.

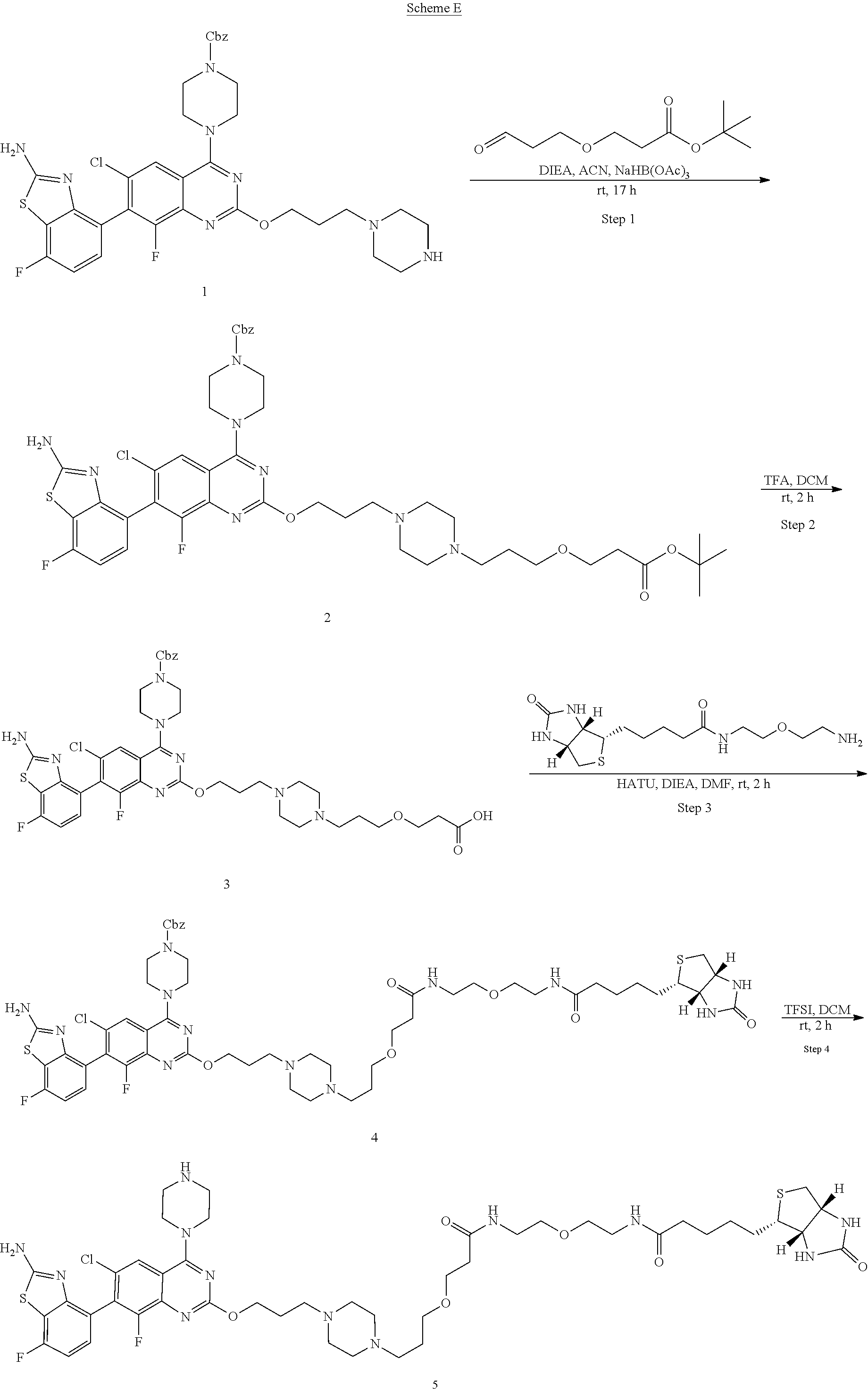

As shown in Scheme E, in certain embodiments, provided herein are methods for preparing N-(2-(2-(3-(3-(4-(3-((7-(2-amino-7-fluorobenzo[d]thiazol-4-yl)-6-chloro-8-fluoro-4-(piperazin-1-yl)quinazolin-2-yl)oxy)propyl)piperazin-1-yl)propoxy)propanamido)ethoxy)ethyl)-5-((3aS,4S,6aR)-2-oxohexahydro-1H-thieno[3,4-d]imidazol-4-yl)pentanamide (5), comprising:
(1) contacting benzyl 4-(7-(2-amino-7-fluorobenzo[d]thiazol-4-yl)-6-chloro-8-fluoro-2-(3-(piperazin-1-yl)propoxy)quinazolin-4-yl)piperazine-1-carboxylate (1) in CH₃CN in the presence of DIPEA and NaHB(AcO)₃ at room temperature; and
(2) contacting 3-(3-(4-(3-((7-(2-amino-7-fluorobenzo[d]thiazol-4-yl)-4-(4-((benzyloxy)carbonyl)piperazin-1-yl)-6-chloro-8-fluoroquinazolin-2-yl)oxy)propyl)piperazin-1-yl)propoxy)propanoic acid (3), N-(2-(2-aminoethoxy)ethyl)-5-((3aS,4S,6aR)-2-oxohexahydro-1H-thieno[3,4-d]imidazol-4-yl)pentanamide and HATU in DMF in the presence of DIPEA at room temperature.

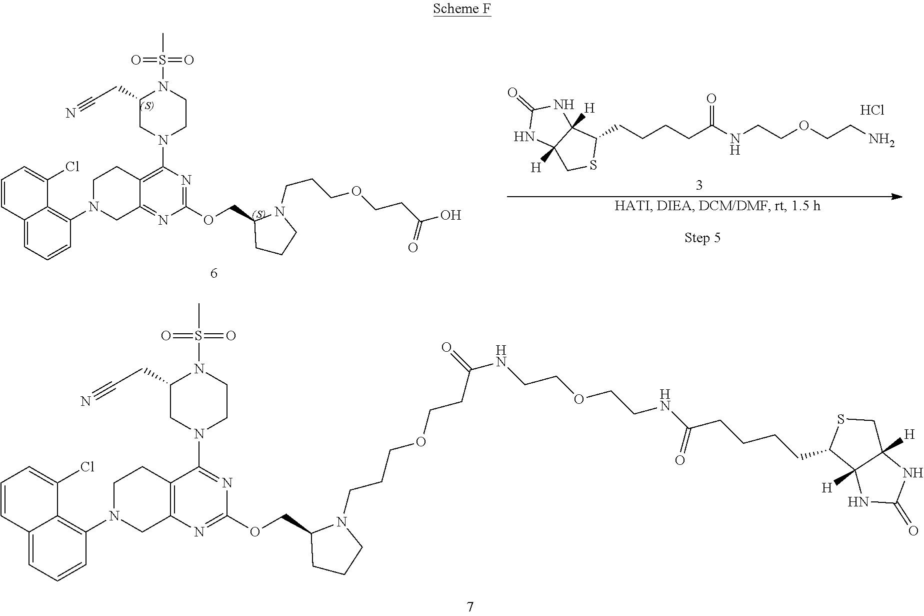

As shown in Scheme F, in certain embodiments, provided herein are methods for preparing N-(2-(2-(3-(3-((S)-2-(((7-(8-chloronaphthalen-1-yl)-4-((S)-3-(cyanomethyl)-4-(methylsulfonyl)piperazin-1-yl)-5,6,7,8-tetrahydropyrido[3,4-d]pyrimidin-2-yl)oxy)methyl)pyrrolidin-1-yl)propoxy)propanamido)ethoxy)ethyl)-5-((3aS,4S,6aR)-2-oxohexahydro-H-thieno[3,4-d]imidazol-4-yl)pentanamide (7), comprising:
contacting 3-(3-((S)-2-(((7-(8-chloronaphthalen-1-yl)-4-((S)-3-(cyanomethyl)-4-(methylsulfonyl)piperazin-1-yl)-5,6,7,8-tetrahydropyrido[3,4-d]pyrimidin-2-yl)oxy)methyl)pyrrolidin-1-yl)propoxy)propanoic acid (6), N-(2-(2-aminoethoxy)ethyl)-5-((3aS,4S,6aR)-2-oxohexahydro-1H-thieno[3,4-d]imidazol-4-yl)pentanamide hydrochloride and HATU in a mixture of DCM and DMF in the presence of DIPEA at room temperature.

### Methods of Use

Provided herein is a method of inhibiting KRAS G12D activity, which comprises administering to an individual the compound disclosed herein, or a pharmaceutically acceptable salt thereof, including the compound of formulas (I) or (II) or the specific compounds exemplified herein.

Provided herein is a method of treating a disease or disorder in a patient comprising administering to the patient a therapeutically effective amount of the compound disclosed herein, or a pharmaceutically acceptable salt thereof as a KRAS G12D inhibitor, wherein the compound disclosed herein includes the compound of formula (I) or the specific compounds exemplified herein. In some embodiments, the disease or disorder is associated with inhibition of KRAS G12D interaction. Preferably, the disease or disorder is pancreatic cancer.

Provided herein is a bifunctional compound composed of a target protein (i.e., KRAS G12D)-binding moiety and an E3 ubiquitin ligase-binding moiety, which has been shown to induce proteasome-mediated degradation of selected proteins. In some embodiments, the bifunctional compound disclosed herein is composed of a target protein (i.e., KRAS G12D)-binding moiety disclosed herein and an E3 ubiquitin ligase-binding moiety known in the art. In some embodiments, disclosed herein is the use of the compound disclosed herein in the preparation of degrading a target protein compound by using chemical modification of the compound disclosed herein.

In one aspect, provided herein are methods for treating or preventing pancreatic cancer, comprising administering to a subject in need thereof an effective amount of a Bridged Compound. Also provided herein are methods for treating or preventing a condition treatable or preventable by inhibition of a RAS/MAPK pathway, comprising administering to a subject in need thereof an effective amount of a Bridged Compound.

In one aspect, provided herein are methods for inhibiting the function of a protein, for example KRAS protein, in a cell expressing said protein, comprising contacting said cell with an effective amount of a Bridged Compound. In one aspect, provided herein are methods for inhibiting the function of a protein, for example KRas protein with G12D mutation, in a cell expressing said protein, comprising contacting said cell with an effective amount of a Bridged Compound.

A Bridged Compound is useful for treating or preventing various diseases, e.g., pancreatic cancer, as described herein. In another aspect, a Bridged Compound is useful for treating or preventing pancreatic cancer as described herein. In another aspect, a Bridged Compound is useful for treating or preventing a condition treatable or preventable by inhibition of the function of KRAS protein, as described herein. In another aspect, a Bridged Compound is useful for treating or preventing a condition treatable or preventable by inhibition of the function of KRAS protein with G12D mutation, as described herein. In another aspect, a Bridged Compound is useful for treating or preventing a condition treatable or preventable by inhibition of a RAS/MAPK pathway, as described herein.

Provided herein is a method of testing biological activity of a KRAS inhibitor, comprising
(i) incubating a KRAS protein with the to-be-tested compound in a solution;
(ii) adding a biotinylated probe (I) as depicted in Example 57 for KRAS protein to the system of (i) following the incubation resulting a solution,
wherein the KI Moiety is a KRAS inhibitor moiety;
   the Linker moiety is a divalent linker between KI Moiety and Biotin;
   the Biotin is a biotin moiety linked to the Linker moiety with an amide group or ester group;
(iii) adding Mab Anti-GST-Tb cryptate and Streptavidin-XL665 to the system of (ii) resulting a solution;
(iv) reading two different wavelength TR-FRET signals emitted by the system of (iii) while using a light as incident light; and
wherein the biological activity of the KRAS inhibitor are calculated based on the ratio of the two different wavelength TR-FRET signals.

In one embodiment, the KRAS protein is a GDP-loaded KRAS protein; preferably the KRAS protein is a WT KRAS protein or KRAS G12D protein; preferably the KRAS protein is a GST-tagged GDP-loaded WT KRAS or GST-tagged GDP-loaded KRAS G12D; preferably the KRAS protein is a protein or an isoform thereof expressed by a KRAS gene.

In one embodiment, the WT KRAS protein concentration is 1-5 nM or the KRAS G12D protein concentration is 0.1-0.9 nM; preferably, the WT KRAS protein concentration is 2-4 nM or the KRAS G12D protein concentration is 0.3-0.8 nM; preferably the WT KRAS protein concentration is 3 nM or the KRAS G12D protein concentration is 0.5 nM.

In one embodiment, the KI Moiety is a wild type KRAS inhibitor moiety or a KRAS G12D inhibitor moiety; preferably the KI Moiety is

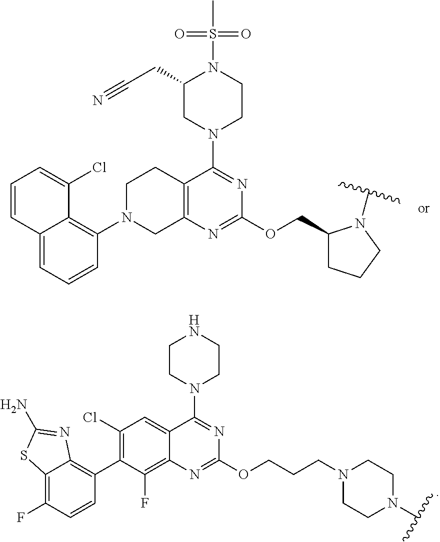 [SMILES: *N1CCC[C@H]1COc1nc2c(c(N3CCN(S(C)(=O)=O)[C@@H](CC#N)C3)n1)CCN(c1cccc3cccc(Cl)c13)C2 ; *N1CCN(CCCOc2nc(N3CCNCC3)c3cc(Cl)c(-c4ccc(F)c5sc(N)nc45)c(F)c3n2)CC1]

In one embodiment, the Linker moiety is

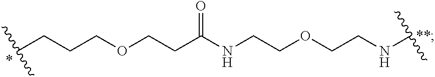 [SMILES: *CCCOCCC(=O)NCCOCCN*]

wherein * refers to the position attached to the KI Moiety, and ** refers to the position attached to the Biotin moiety.

In one embodiment, the Biotin moiety is

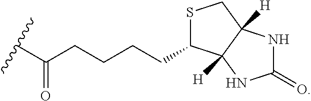 [SMILES: *C(=O)CCCC[C@@H]1SC[C@@H]2NC(=O)N[C@@H]21]

In one embodiment, the biotinylated probe (I) is

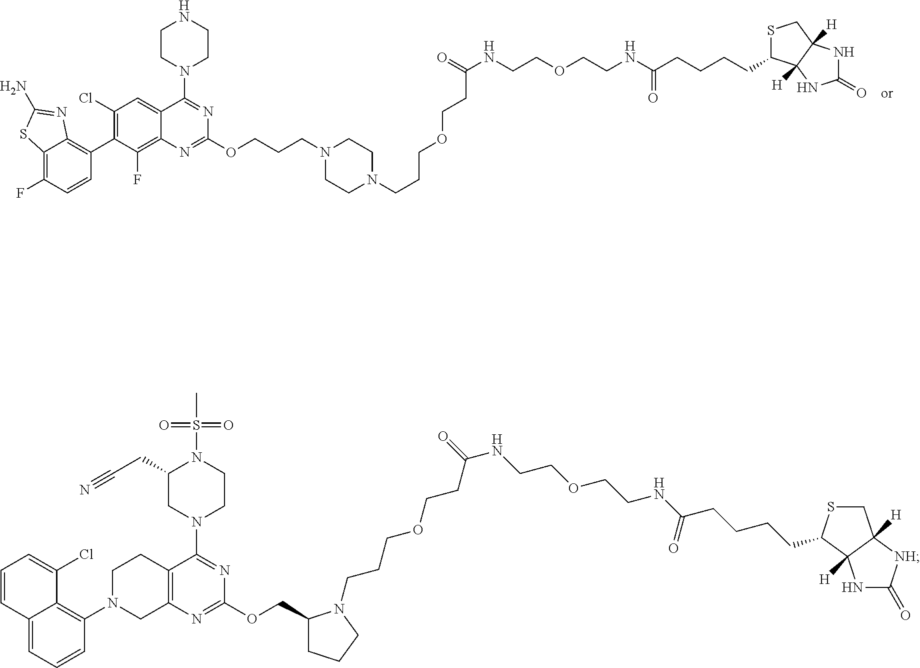 [SMILES: Nc1nc2c(-c3c(Cl)cc4c(N5CCNCC5)nc(OCCCN5CCN(CCCOCCC(=O)NCCOCCNC(=O)CCCC[C@@H]6SC[C@@H]7NC(=O)N[C@@H]76)CC5)nc4c3F)ccc(F)c2s1 ; CS(=O)(=O)N1CCN(c2nc(OC[C@@H]3CCCN3CCCOCCC(=O)NCCOCCNC(=O)CCCC[C@@H]3SC[C@@H]4NC(=O)N[C@@H]43)nc3c2CCN(c2cccc4cccc(Cl)c24)C3)C[C@@H]1CC#N]

preferably wherein the concentration of

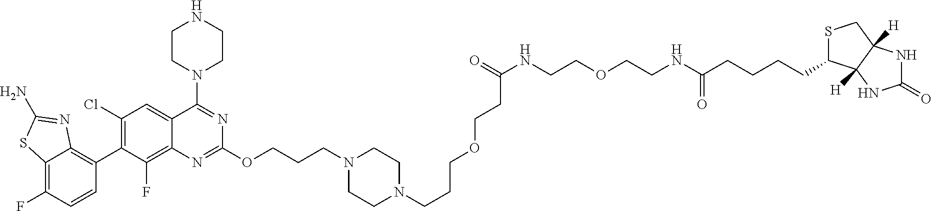 [SMILES: Nc1nc2c(-c3c(Cl)cc4c(N5CCNCC5)nc(OCCCN5CCN(CCCOCCC(=O)NCCOCCNC(=O)CCCC[C@@H]6SC[C@@H]7NC(=O)N[C@@H]76)CC5)nc4c3F)ccc(F)c2s1]

is 2-6 nM; or the concentration of

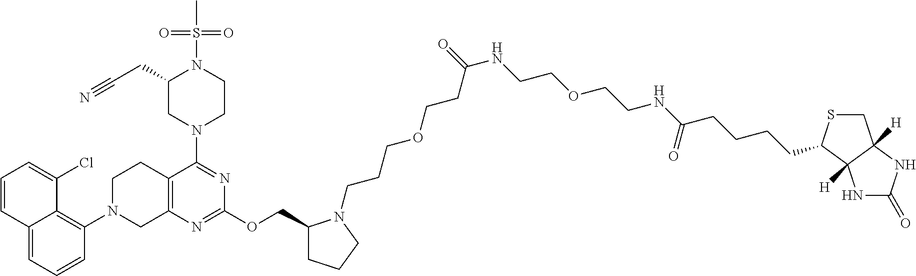 [SMILES: CS(=O)(=O)N1CCN(c2nc(OC[C@@H]3CCCN3CCCOCCC(=O)NCCOCCNC(=O)CCCC[C@@H]3SC[C@@H]4NC(=O)N[C@@H]43)nc3c2CCN(c2cccc4cccc(Cl)c24)C3)C[C@@H]1CC#N]

is 40-80 nM; preferably wherein the concentration of

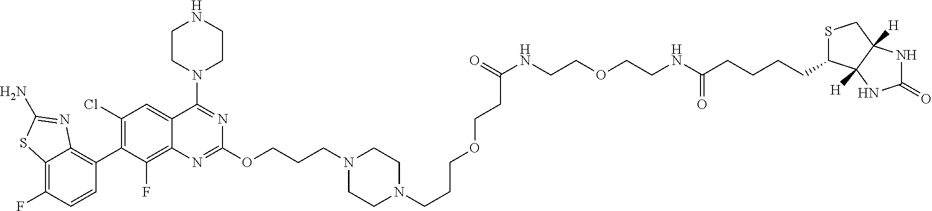 [SMILES: Nc1nc2c(-c3c(Cl)cc4c(N5CCNCC5)nc(OCCCN5CCN(CCCOCCC(=O)NCCOCCNC(=O)CCCC[C@@H]6SC[C@@H]7NC(=O)N[C@@H]76)CC5)nc4c3F)ccc(F)c2s1]

is 4 nM; or the concentration of

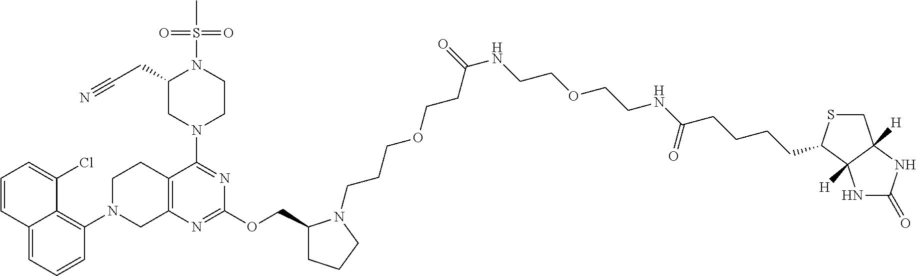 [SMILES: CS(=O)(=O)N1CCN(c2nc(OC[C@@H]3CCCN3CCCOCCC(=O)NCCOCCNC(=O)CCCC[C@@H]3SC[C@@H]4NC(=O)N[C@@H]43)nc3c2CCN(c2cccc4cccc(Cl)c24)C3)C[C@@H]1CC#N]

is 60 nM.

In one embodiment, the wavelengths of the two TR-FRET signals emitted in step (iv) are 640-690 nm/600-640 nm; preferably the two wavelengths are 650-680 nm/610-630 nm; preferably the two wavelengths are 665 nm/620 nm; and the wavelength of the incident light in step (iv) is 300-374 nm, preferably the wavelength of incident light of step (iv) is 327-347 nm, preferably the wavelength of incident light of step (iv) is 337 nm.

In one embodiment, the solutions of steps (i), (ii) and (iii) are prepared in a buffer; preferably the solutions are prepared in a buffer containing 50 mM HEPES pH7.5, 50 mM NaCl, 1 mM MgCl₂, 1 mM TCEP, 0.01% BSA, and 0.008% Brij-35.

In one embodiment, the solution of step (i) is incubated at 20-30° C. for 0.5-1.5 hr; preferably the solution of step (i) is incubated at 24° C. for 1 hr; and/or the solution of step (ii) is incubated at 20-30° C. for 0.5-1.5 hr; preferably the solution of step (ii) is incubated at 24° C. for 1 hr; and/or the solution of (iii) is incubated at 20-30° C. for 0.5-1.5 hr; preferably the solution of step (iii) is incubated at 24° C. for 1 hr.

### EXAMPLES

The following Examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless otherwise indicated. The compounds provided herein are named using the automatic name generating tool provided in Chemdraw Ultra 9.0 (Cambridgesoft), which generates systematic names for chemical structures, with support for the Cahn-Ingold-Prelog rules for stereochemistry. One skilled in the art can modify the procedures set forth in the illustrative examples to arrive at the desired products.

Unless otherwise indicated, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

Unless otherwise indicated, column chromatography purification was conducted on a Biotage system (Manufacturer: Dyax Corporation) having a silica gel column or on a silica SepPak cartridge (Waters), or was conducted on a Teledyne Isco Combiflash purification system using prepacked silica gel cartridges.

¹H NMR spectra were recorded on a Varian instrument operating at 400 MHz or 500 MHz. ¹H-NMR spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDC₃: 7.25 ppm; CD₃OD: 3.31 ppm; D₂O: 4.79 ppm; d₆-DMSO: 2.50 ppm; d₆-acetone: 2.05; (CD₃)₂CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz). All compound names except the reagents were generated by ChemDraw version 18.0.

In the following Examples, the abbreviations below are used:

| | |
|---|---|
| Ac | Acetyl |
| AcOH | Acetic acid |
| Aq | Aqueous |
| BCA | Bicinchoninic Acid |
| Brine | Saturated aqueous sodium chloride solution |
| Bn | Benzyl |
| BnBr | Benzyl Bromide |
| Boc | Tert-butyloxycarbonyl |
| BSA | Bovine serum albumin |
| Cbz | benzyloxycarbonyl |
| CH₂Cl₂ | Dichloromethane |
| DMF | N,N-Dimethylformamide |
| Dppf | 1,1″-bis(diphenylphosphino)ferrocene |
| DPBS | Dulbecco's Phosphate Buffered Saline |
| DCM | Dichloromethane |
| DIEA or DIPEA | N,N-diisopropylethylamine, N-ethyl-N- |
| | isopropylpropan-2-amine |
| DMSO | Dimethyl sulfoxide |
| EA or EtOAc | Ethyl acetate |
| ESI | electrospray ionization |
| EtOH | Ethanol |
| Et₂O or ether | Diethyl ether |
| Et₃N | Triethylamine |
| FA | Formic acid |
| G | Grams |
| GDP | Guanosine diphosphate |
| GST | Glutathione S-transferase |
| h or hr | Hour |
| HATU | O-(7-Azabenzotriazol-1-yl)-N,N,N′,N′- |
| | tetramethyluronium hexafluorophosphate |
| HBTU | O-(7-Azabenzotriazol-1-yl)-N,N,N′,N′- |
| | tetramethyluronium hexafluorophosphate |
| HCl | Hydrochloric acid |
| HPLC | High-performance liquid chromatography |
| i-PrOH | Isopropyl alcohol |
| LHMDS | Lithium bis(trimethylsilyl)amide |
| mg | Milligrams |
| mL | Milliliters |
| Mmol | Millimole |
| MeCN | Acetonitrile |
| MeOH | Methanol |
| Min | Minutes |
| ms or MS | Mass spectrum |
| NMR | Nuclear magnetic resonance |
| Pd/C | Palladium on carbon |
| PBS | Phosphate buffered solution |
| PE | Petroleum ether |
| Ph₃PO | Triphenyl phosphorus oxide |
| PMB | 4-Methoxybenzyl, p-Methoxybenzyl |
| PPh | Triphenylphosphine |
| PTSA | p-toluenesulfonic acid |
| Rt | Retention time |
| Rt., RT. Or rt. | Room temperature |
| RT-FRET | Real Time-Fluorescence Resonance Energy Transfer |
| Ru-Phos/Ru-PHOS | 2-dicyclohexylphosphino-2′,6′-di-i-propoxy- |
| | 1,1′-biphenyl |
| Sn(Bu)3 | tributylstannyl |
| STAB | Sodium triacetoxyborohydride |
| TBAI | Tetrabutylammonium iodide |
| TFA | Trifluoroacetic acid |
| THF | Tetrahydrofuran |
| TBST | Tris buffered saline with Tween 20 |
| μL | Microliters |
| UV | Ultraviolet |
| WT | Wild Type |

### Compound Synthesis

### Example 1: 4-(6-chloro-2-(3-(dimethylamino)propoxy)-8-fluoro-4-(piperazin-1-)quinazolin-7-yl)naphthalen-2-ol

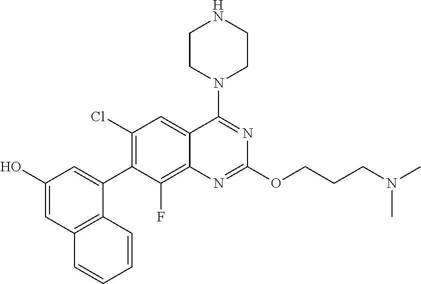 [SMILES: CN(C)CCCOc1nc(N2CCNCC2)c2cc(Cl)c(-c3cc(O)cc4ccccc34)c(F)c2n1]

Synthetic Route

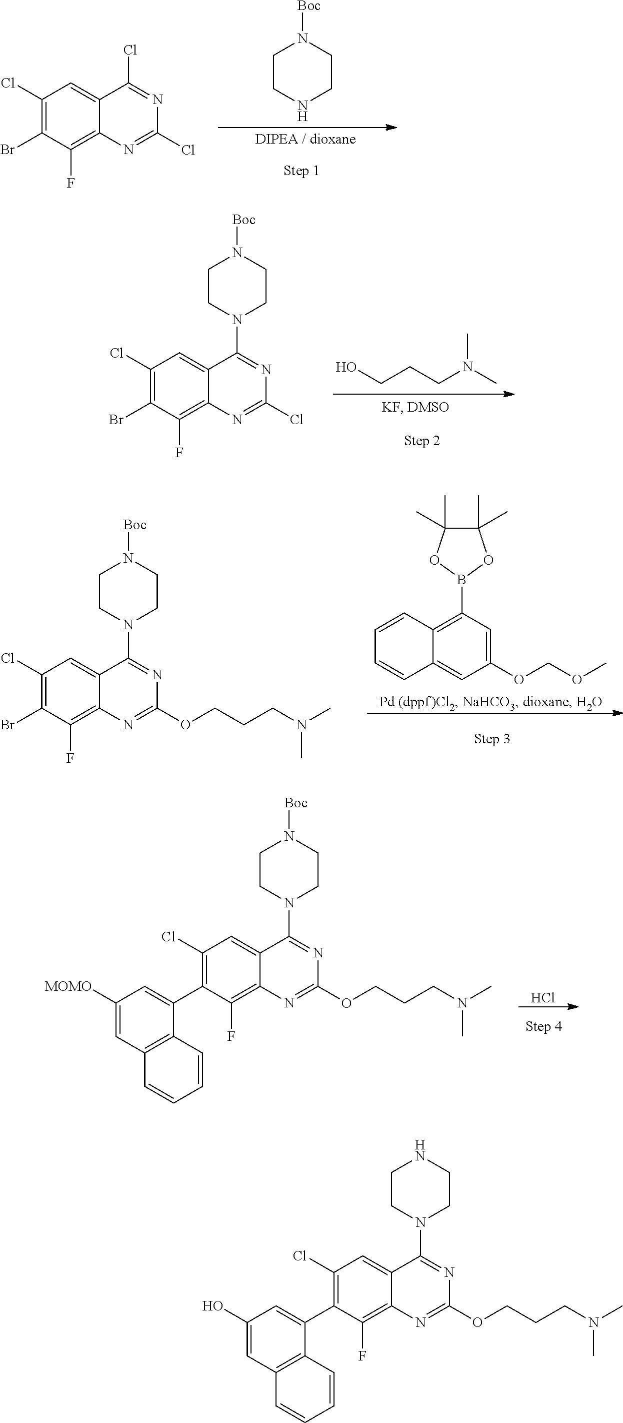

### Step 1: tert-butyl 4-(7-bromo-2,6-dichloro-8-fluoroquinazolin-4-yl)piperazine-1-carboxylate

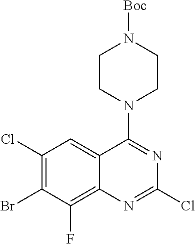 [SMILES: CC(C)(C)OC(=O)N1CCN(c2nc(Cl)nc3c(F)c(Br)c(Cl)cc23)CC1]

To a solution of 7-bromo-2,4,6-trichloro-8-fluoroquinazoline (4.0 g, 12.1 mmol), tert-butyl piperazine-1-carboxylate (2.25 g, 12.1 mmol) in dioxane (50 mL) was added N-ethyl-N-isopropylpropan-2-amine (3.1 g, 24.2 mmol) for 2 min at 0° C. and the reaction mixture was stirred for 5 h at room temperature. After completion of the reaction, brine (50 mL) was added to the reaction and the resulting mixture was extracted with EtOAc (150 mL). The organic phase was washed with brine (100 mL×3), dried over anhydrous Na₂SO₄ and the solvent was removed. The crude product was purified by chromatography on silica (PE/EtOAc=10/1) to give the title compound (4 g, 69%). MS (ESI, m/e) [M+H]⁺ 481.2.

### Step 2: tert-butyl 4-(7-bromo-6-chloro-2-(3-(dimethylamino)propoxy)-8-fluoroquinazolin-4-yl)piperazine-1-carboxylate

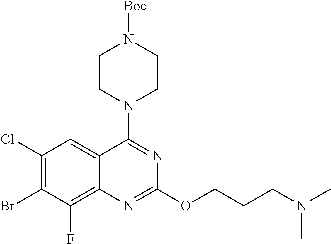 [SMILES: CN(C)CCCOc1nc(N2CCN(C(=O)OC(C)(C)C)CC2)c2cc(Cl)c(Br)c(F)c2n1]

To a solution of 3-(dimethylamino)propan-1-ol (1.72 g, 16.67 mmol), tert-butyl 4-(7-bromo-2,6-dichloro-8-fluoroquinazolin-4-yl)piperazine-1-carboxylate (4 g, 8.33 mmol) in dry DMSO (20 mL) was added KF (1.45 g, 25 mmol). The resulting mixture was stirred for 2 h at 100° C. Brine (20 mL) was added and the resulting mixture was extracted with EtOAc (100 mL). The organic phase was washed with brine (3×50 mL), dried over anhydrous Na₂SO₄ and the solvent was removed. The crude product was purified by chromatography on silica (DCM/MeOH=10/1) to give the title compound (3.2 g, 71%). MS (ESI, m/e) [M+1]⁺546.1.

### Step 3: tert-butyl 4-(6-chloro-2-(3-(dimethylamino)propoxy)-8-fluoro-7-(3-(methoxymethoxy)naphthalen-1-yl)quinazolin-4-yl)piperazine-1-carboxylate

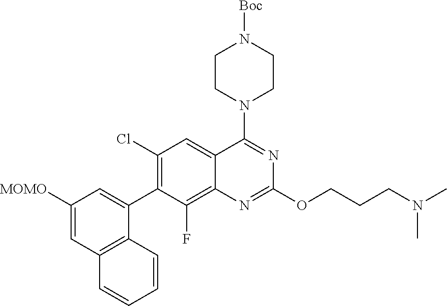

The solution of methyl tert-butyl 4-(7-bromo-6-chloro-2-(3-(dimethylamino)propoxy)-8-fluoroquinazolin-4-yl)piperazine-1-carboxylate (150 mg, 0.27 mmol), 2-(3-(methoxymethoxy)naphthalen-1-yl)-4,4,5,5-tetramethyl-1,3,2-dioxaborolane (86 mg, 0.27 mmol), Pd(dppf)Cl₂ (40 mg, 0.055 mmol), and NaHCO₃ (58 mg, 0.55 mmol) in dioxane (10 mL) and H₂O (2 mL) was stirred for 2 h at 100° C. Brine (10 mL) was added to the reaction and the resulting mixture was extracted with EtOAc (60 mL). The organic phase was washed with brine (3×20 mL), dried over anhydrous Na₂SO₄ and the solvent was removed. The crude product was purified by chromatography on silica (DCM/MeOH=10/1) to give the title compound (50 mg, 28%). MS (ESI, m/e) [M+1]⁺655.2.

### Step 4: 4-(6-chloro-2-(3-(dimethylamino)propoxy)-8-fluoro-4-(piperazin-1-yl)quinazolin-7-yl)naphthalen-2-ol

To a solution of tert-butyl 4-(6-chloro-2-(3-(dimethylamino)propoxy)-8-fluoro-7-(3-(methoxymethoxy)naphthalen-1-yl)quinazolin-4-yl)piperazine-1-carboxylate (50 mg, 0.076 mmol) in DCM (5 mL) was added HCl (4N in dioxane, 5 mL). The mixture was stirred at room temperature for 5 hrs. The solution was concentrated and diluted with MeOH (10 mL). Then Na₂CO₃ (100 mg) was added and the mixture was stirred for 1 h. Solid was filtrated off and the organic layer was purified by Prep-HPLC to give the title product (5 mg, 13%). ¹H NMR (400 MHz, DMSO-d₆) δ 8.24-8.19 (m, 1H), 7.98-7.92 (m, 1H), 7.83-7.77 (m, 1H), 7.47-7.39 (m, 1H), 7.30-7.26 (m, 1H), 7.25-7.17 (m, 2H), 7.09-7.04 (m, 1H), 4.43-4.30 (m, 2H), 3.84-3.74 (m, 4H), 3.01-2.92 (m, 4H), 2.46-2.38 (m, 2H), 2.19 (s, 6H), 1.95-1.82 (m, 2H). MS (ESI, m/e) [M+H]⁺ 510.2.

### Example 2: 4-(6-chloro-2-(3-(dimethylamino)azetidin-1-yl)-8-fluoro-4-(piperazin-1-yl)quinazolin-7-yl)naphthalen-2-ol

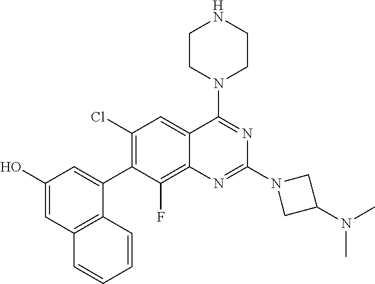 [SMILES: CN(C)C1CN(c2nc(N3CCNCC3)c3cc(Cl)c(-c4cc(O)cc5ccccc45)c(F)c3n2)C1]

### Step 1: tert-butyl 4-(7-bromo-6-chloro-2-(3-(dimethylamino)azetidin-1-yl)-8-fluoroquinazolin-4-yl)piperazine-1-carboxylate

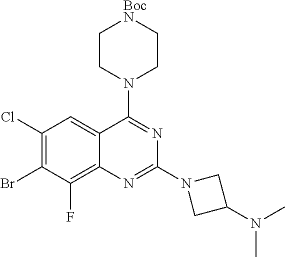 [SMILES: CN(C)C1CN(c2nc(N3CCN(C(=O)OC(C)(C)C)CC3)c3cc(Cl)c(Br)c(F)c3n2)C1]

To a mixture of tert-butyl 4-(7-bromo-2,6-dichloro-8-fluoroquinazolin-4-yl)piperazine-1-carboxylate (100 mg, 0.21 mmol) and N,N-dimethylazetidin-3-amine hydrochloride (54 g, 0.31 mmol) in DMSO (100 mL) was added KF (58 g, 0.63 mmol). The mixture was stirred for 16 hrs at room temperature. The reaction mixture was diluted with EtOAc and water, extracted with EtOAc twice. Combined organic layer was dried over sodium sulfate and evaporated. The residue was purified by chromatography column on silica (eluting with MeOH/DCM=5-7%) to give the title product (100 mg, 87.7%). MS (ESI, m/e) [M+1]⁺544.8.

### Step 2: tert-butyl 4-(6-chloro-2-(3-(dimethylamino)azetidin-1-yl)-8-fluoro-7-(3-(methoxymethoxy)naphthalen-1-yl)quinazolin-4-yl)piperazine-1-carboxylate

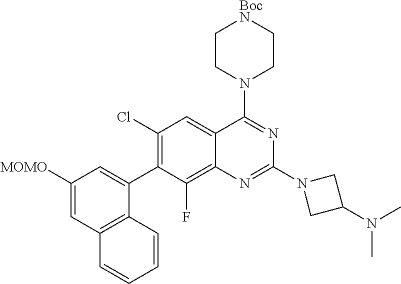

To a solution of tert-butyl 4-(7-bromo-6-chloro-2-(3-(dimethylamino)azetidin-1-yl)-8-fluoroquinazolin-4-yl)piperazine-1-carboxylate (100 mg, 0.185 mmol) and 2-(3-(methoxymethoxy)naphthalen-1-yl)-4,4,5,5-tetramethyl-1,3,2-dioxaborolane (85 mg, 0.27 mmol) in dioxane (10 mL) was added 1,1′-Bis(diphenylphosphino)ferrocene-palladium(II)dichloride (14 mg, 2.45 mmol) and NaHCO₃/H₂O (47 mg/2 mL), then stirred at 90° C. for 3 hrs. The mixture was then concentrated and diluted with EtOAc and water, extracted with EtOAc twice, and the organic layer was combined, dried over sodium sulfate and evaporated. The residue was purified by chromatography column on silica (eluting with MeOH/DCM=10%) to give the title product (50 mg, 41.5%). MS (ESI, m/e) [M+1]⁺651.3.

### Step 3: 4-(6-chloro-2-(3-(dimethylamino)azetidin-1-yl)-8-fluoro-4-(piperazin-1-yl)quinazolin-7-yl)naphthalen-2-ol

To a solution of tert-butyl 4-(6-chloro-2-(3-(dimethylamino)azetidin-1-yl)-8-fluoro-7-(3-(methoxymethoxy)naphthalen-1-yl)quinazolin-4-yl)piperazine-1-carboxylate (50 mg, 0.077 mmol) in EtOAc (20 mL) was added HCl/dioxane (2 mL, 4 N). The mixture was stirred for 4 hrs at room temperature. Then, the resulting mixture was filtered and the filter cake was dissolved with water, pH was adjusted to 9 and extracted with DCM/MeOH (v/v=10/1), and the organic layer was combined. Solvent were removed and the residue was purified by chromatography column on silica (eluting with MeOH/DCM=20-25%) to give the title product (33 mg, 85%). ¹H NMR (400 MHz, CD₃OD) δ 8.13 (s, 1H), 7.77 (d, J=8.2 Hz, 1H), 7.44 (s, 1H), 7.27 (d, J=26.5 Hz, 3H), 7.06 (s, 1H), 4.80-4.68 (m, 4H), 4.51-4.31 (m, 5H), 3.62-3.46 (m, 4H), 2.96 (s, 6H). MS (ESI, m/e) [M+H]⁺ 507.5.

### Example 3:4-(4-(2,5-diazabicyclo[2.2.1]heptan-2-yl)-6-chloro-2-(3-(dimethylamino)propoxy)-8-fluoroquinazolin-7-yl)-7-fluorobenzo[d]thiazol-2-amine

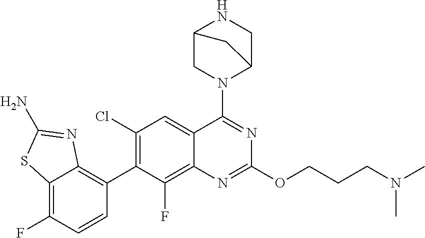 [SMILES: CN(C)CCCOc1nc(N2CC3CC2CN3)c2cc(Cl)c(-c3ccc(F)c4sc(N)nc34)c(F)c2n1]

Example 3 was prepared by similar procedure as described in Example 4 from tert-butyl 2,5-diazabicyclo[2.2.1]heptane-2-carboxylate. ¹H NMR (400 MHz, CD₃OD) δ 8.01 (s, 1H), 7.21 (dd, J=8.3, 5.4 Hz, 1H), 6.99 (t, J=8.8 Hz, 1H), 5.38 (s, 1H), 4.63-4.38 (m, 4H), 4.06 (d, J=10.5 Hz, 1H), 3.62 (d, J=10.1 Hz, 1H), 3.46 (d, J=9.7 Hz, 1H), 3.30-3.26 (m, 2H), 2.89 (s, 6H), 2.46-2.07 (m, 4H). MS (ESI, m/e) [M+1]⁺546.4.

### Example 4: 4-(4-(2,5-diazabicyclo[2.2.2] octan-2-yl)-6-chloro-2-(3-(dimethylamino) propoxy)-8-fluoroquinazolin-7-yl)-7-fluorobenzo[d]thiazol-2-amine

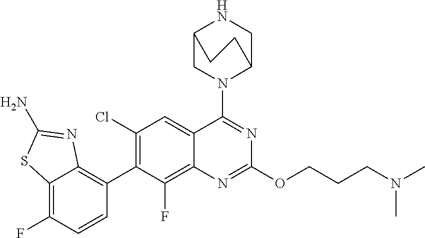 [SMILES: CN(C)CCCOc1nc(N2CC3CCC2CN3)c2cc(Cl)c(-c3ccc(F)c4sc(N)nc34)c(F)c2n1]

Synthetic Route

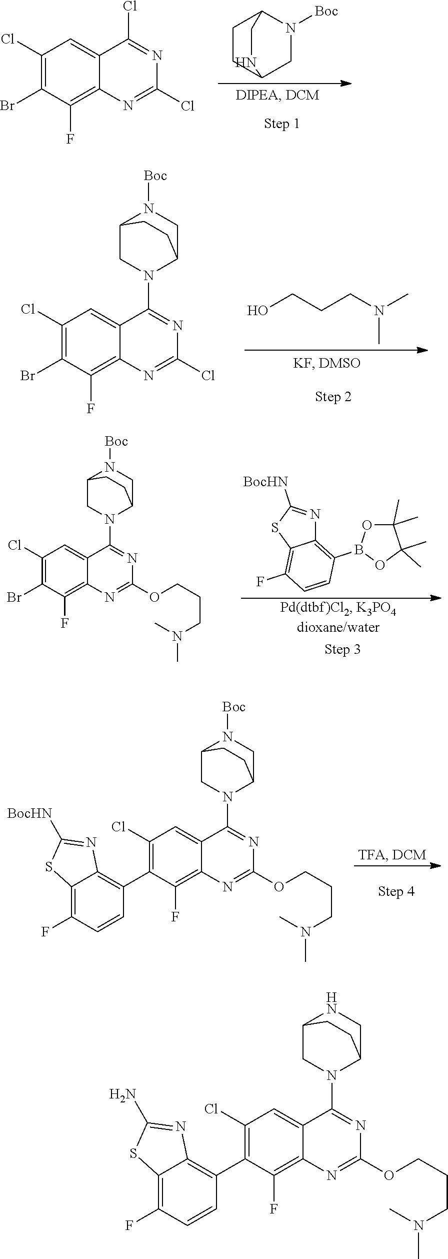

### Step 1: tert-butyl 5-(7-bromo-2,6-dichloro-8-fluoroquinazolin-4-yl)-2,5-diazabicyclo [2.2.2] octane-2-carboxylate

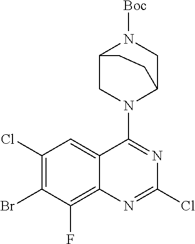 [SMILES: CC(C)(C)OC(=O)N1CC2CCC1CN2c1nc(Cl)nc2c(F)c(Br)c(Cl)cc12]

To a solution of 7-bromo-2,4,6-trichloro-8-fluoroquinazoline (300 mg, 0.91 mmol) in DCM (30 mL) was added tert-butyl 2,5-diazabicyclo [2.2.2] octane-2-carboxylate (290 mg, 1.365 mmol) and Et₃N (276 mg, 2.73 mmol), and the mixture was stirred at room temperature for 1 h. The resulting mixture was concentrated and the crude product was purified by chromatography column on silica (eluting with PE/EtOAc=5/1) to give the title product (370 mg, 80%). MS (ESI, m/e) [M+1]⁺507.0.

### Step 2: tert-butyl 5-(7-bromo-6-chloro-2-(3-(dimethylamino) propoxy)-8-fluoroquinazolin-4-yl)-2,5-diazabicyclo [2.2.2] octane-2-carboxylate

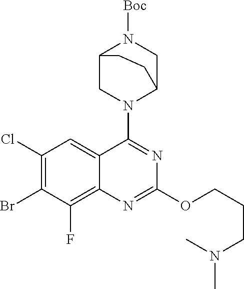 [SMILES: CN(C)CCCOc1nc(N2CC3CCC2CN3C(=O)OC(C)(C)C)c2cc(Cl)c(Br)c(F)c2n1]

To a solution of tert-butyl 5-(7-bromo-2,6-dichloro-8-fluoroquinazolin-4-yl)-2,5-diazabicyclo [2.2.2] octane-2-carboxylate (370 mg, 0.73 mmol) in DMSO (20 mL) was added 3-(dimethylamino) propan-1-ol (300.7 mg, 2.92 mmol) and KF (254 mg, 4.38 mmol), and the mixture was stirred at 60° C. for overnight. The resulting mixture was diluted with water (30 mL) and extracted with DCM (60 ml×3). Combined organic layer was concentrated and purified by chromatography column on silica (eluting with PE/EtOAc=5/1) to give the title product (370 mg, 88%). MS (ESI, m/e) [M+1]⁺574.1.

### Step 3: tert-butyl 5-(7-(2-((tert-butoxycarbonyl)amino)-7-fluorobenzo[d]thiazol-4-yl)-6-chloro-2-(3-(dimethylamino)propoxy)-8-fluoroquinazolin-4-yl)-2,5-diazabicyclo[2.2.2]octane-2-carboxylate

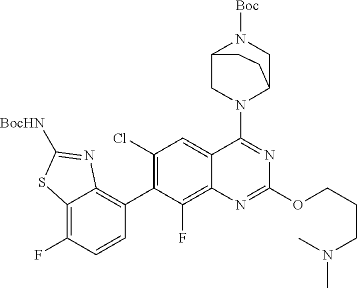 [SMILES: CN(C)CCCOc1nc(N2CC3CCC2CN3C(=O)OC(C)(C)C)c2cc(Cl)c(-c3ccc(F)c4sc(NC(=O)OC(C)(C)C)nc34)c(F)c2n1]

To a solution of tert-butyl 5-(7-bromo-6-chloro-2-(3-(dimethylamino) propoxy)-8-fluoroquinazolin-4-yl)-2,5-diazabicyclo [2.2.2] octane-2-carboxylate (200 mg, 0.344 mmol) in dioxane (30 mL) was added tert-butyl (7-fluoro-4-(4,4,5,5-tetramethyl-1,3,2-dioxaborolan-2-yl)benzo[d]thiazol-2-yl)carbamate (220 mg, 0.556 mmol), Pd(dppf)Cl₂ (28 mg, 0.0344 mmol), NaHCO₃ (88 mg, 1.044 mmol) and water (6 mL) at room temperature, and the mixture was stirred at 100° C. for 2 hrs. The resulting mixture was concentrated and the crude product was purified by chromatography column on silica (eluting with DCM/MeOH=20/1) to give the crude product which was used in the next step without further purification. MS (ESI, m/e) [M+1]⁺760.3.

### Step 4: 4-(4-(2,5-diazabicyclo [2.2.2] octan-2-yl)-6-chloro-2-(3-(dimethylamino) propoxy)-8-fluoroquinazolin-7-yl)-7-fluorobenzo[d]thiazol-2-amine

To a solution of tert-butyl 5-(7-(2-((tert-butoxycarbonyl)amino)-7-fluorobenzo[d]thiazol-4-yl)-6-chloro-2-(3-(dimethylamino)propoxy)-8-fluoroquinazolin-4-yl)-2,5-diazabicyclo[2.2.2]octane-2-carboxylate (300 mg, 0.4 mmol) in DCM (30 mL) was added TFA (6 mL), and the mixture was stirred at room temperature for 1 h. The resulting mixture was concentrated and pH was adjusted to 7 with Na₂CO₃. The organic layer was concentrated to give a residue which was further purified by Prep-HPLC to give title product (23 mg, 10%). H NMR (400 MHz, CD₃OD) δ 8.05 (s, 1H), 7.21 (m, 4.8 Hz, 1H), 7.00 (t, J=8.8 Hz, 1H), 5.00 (s, 1H), 4.53 (t, J=5.8 Hz, 2H), 4.43 (d, J=11.9 Hz, 1H), 4.28 (d, J=11.9 Hz, 1H), 3.84-3.67 (m, 2H), 3.47 (d, J=12.6 Hz, 1H), 3.27 (t, J=7.4 Hz, 2H), 2.88 (s, 6H), 2.41 (m, 1H), 2.27-2.15 (m, 3H), 2.13-1.96 (m, 2H). MS (ESI, m/e) [M+1]⁺560.1.

### Example 5: 4-(4-(3,8-diazabicyclo [3.2.1] octan-3-yl)-6-chloro-8-fluoro-2-((1-(piperidin-1-yl) propan-2-yl)_oxy) quinazolin-7-yl)-7-fluorobenzo[d]thiazol-2-amine

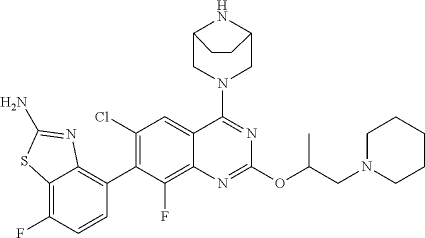 [SMILES: CC(CN1CCCCC1)Oc1nc(N2CC3CCC(C2)N3)c2cc(Cl)c(-c3ccc(F)c4sc(N)nc34)c(F)c2n1]

### Step 1: tert-butyl 3-(7-bromo-2,6-dichloro-8-fluoroquinazolin-4-yl)-3,8-diazabicyclo [3.2.1] octane-8-carboxylate

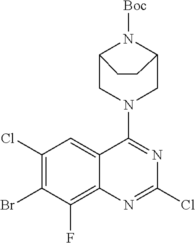 [SMILES: CC(C)(C)OC(=O)N1C2CCC1CN(c1nc(Cl)nc3c(F)c(Br)c(Cl)cc13)C2]

To a solution of 7-bromo-2,4,6-trichloro-8-fluoroquinazoline (500 mg, 1.52 mmol) in DCM (40 mL) was added tert-butyl 3,8-diazabicyclo[3.2.1]octane-8-carboxylate (483 mg, 2.28 mmol) and DIPEA (588 mg, 4.56 mmol), and the mixture was stirred at room temperature for 1 h. The resulting mixture was concentrated and the crude product was purified by chromatography column on silica (eluting with PE/EtOAc=5/1) to give the title product (540 mg, 71%). MS (ESI, m/e) [M+1]⁺507.0.

### Step 2: tert-butyl 3-(7-bromo-6-chloro-2,8-difluoroquinazolin-4-yl)-3,8-diazabicyclo [3.2.1]octane-8-carboxylate

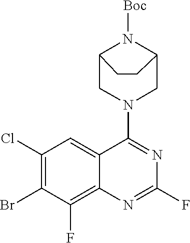 [SMILES: CC(C)(C)OC(=O)N1C2CCC1CN(c1nc(F)nc3c(F)c(Br)c(Cl)cc13)C2]

To a solution of tert-butyl 3-(7-bromo-2,6-dichloro-8-fluoroquinazolin-4-yl)-3,8-diazabicyclo [3.2.1] octane-8-carboxylate (500 mg, 0.99 mmol) in DMSO (20 mL) was added KF (574 mg, 9.9 mmol), and the mixture was stirred at 100° C. for overnight. The resulting mixture was diluted with water (50 mL) and extracted with DCM (60 mL×3), and the combined organic layer was concentrated and purified by chromatography column on silica (eluting with PE/EtOAc=5/1) to give the title product (344 mg, 71%). MS (ESI, m/e) [M+1]⁺491.0.

### Step 3: tert-butyl 3-(7-bromo-6-chloro-8-fluoro-2-((1-(piperidin-1-yl) propan-2-yl)oxy)quinazolin-4-yl)-3,8-diazabicyclo[3.2.1]octane-8-carboxylate

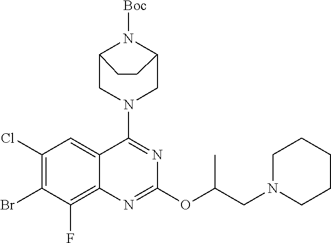 [SMILES: CC(CN1CCCCC1)Oc1nc(N2CC3CCC(C2)N3C(=O)OC(C)(C)C)c2cc(Cl)c(Br)c(F)c2n1]

NaH (58.4 mg, 1.46 mmol) was added to a solution of 1-(piperidin-1-yl) propan-2-ol (300 mg, 0.61 mmol) in THF (15 mL), and the mixture was stirred at 0° C. for 0.5 h. Then a solution of tert-butyl 3-(7-bromo-6-chloro-2,8-difluoroquinazolin-4-yl)-3,8-diazabicyclo [3.2.1] octane-8-carboxylate (300 mg, 0.61 mmol) in THF (10 mL) was added in and the mixture was stirred at room temperature for 1 h. The mixture was then quenched with ice water (15 mL) and extracted with EtOAc (40 mL×3). Combined organic layer was concentrated and purified by chromatography column on silica (eluting with DCM/MeOH=10/1) to give the title product (294 mg, 78%). MS (ESI, m/e) [M+1]⁺614.1.

### Step 4: tert-butyl 3-(7-(2-((tert-butoxycarbonyl)amino)-7-fluorobenzo[d]thiazol-4-yl)-6-chloro-8-fluoro-2-((1-(piperidin-1-yl)propan-2-yl)oxy)quinazolin-4-yl)-3,8-diazabicyclo[3.2.1]octane-8-carboxylate

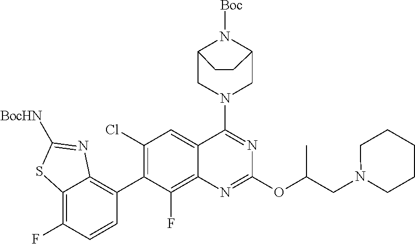 [SMILES: CC(CN1CCCCC1)Oc1nc(N2CC3CCC(C2)N3C(=O)OC(C)(C)C)c2cc(Cl)c(-c3ccc(F)c4sc(NC(=O)OC(C)(C)C)nc34)c(F)c2n1]

To a solution of tert-butyl 3-(7-bromo-6-chloro-8-fluoro-2-((1-(piperidin-1-yl) propan-2-yl)oxy)quinazolin-4-yl)-3,8-diazabicyclo[3.2.1]octane-8-carboxylate (284 mg, 0.463 mmol) in dioxane (30 mL) was added tert-butyl (7-fluoro-4-(4,4,5,5-tetramethyl-1,3,2-dioxaborolan-2-yl)benzo[d]thiazol-2-yl)carbamate (310 mg, 0.787 mmol), Pd(dppf)Cl₂ (38 mg, 0.0463 mmol), NaHCO₃ (117 mg, 1.389 mmol) and water (6 mL), and the mixture was stirred at 100° C. for 2 hrs. The resulting mixture was concentrated and the crude product was purified by chromatography column on silica (eluting with DCM/MeOH=20/1) to give the crude product which was used in the next step without further purification. MS (ESI, m/e) [M+1]⁺800.3.

### Step 5: 4-(4-(3,8-diazabicyclo [3.2.1] octan-3-yl)-6-chloro-8-fluoro-2-((1-(piperidin-1-yl) propan-2-yl) oxy) quinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-6-chloro-8-fluoro-2-((1-(piperidin-1-yl)propan-2-yl)oxy)quinazolin-4-yl)-3,8-diazabicyclo[3.2.1]octane-8-carboxylate (200 mg, 0.25 mmol) in DCM (30 mL) was added TFA (6 mL), and the mixture was stirred at room temperature for 1 h. The resulting mixture was concentrated and pH was adjusted to 7 with Na₂CO₃. The organic layer was concentrated to give a residue which was further purified by Prep-HPLC to give title product (33.5 mg, 22%). ¹H NMR (500 MHz, CD₃OD) δ 7.93 (d, J=3.5 Hz, 1H), 7.21 (ddd, J=10.4, 8.4, 5.4 Hz, 1H), 7.01 (td, J=8.8, 2.9 Hz, 1H), 5.67 (s, 1H), 4.65-4.54 (m, 2H), 4.07 (d, J=20.9 Hz, 2H), 3.80 (m, 2H), 3.50-3.31 (m, 4H), 3.20 (m, 2H), 2.06 (m, 4H), 1.80 (m, 4H), 1.63 (m, 2H), 1.47 (dd, J=6.2, 2.5 Hz, 3H). MS (ESI, m/e) [M+1]⁺600.2.

### Example 5i: tert-butyl (7-fluoro-4-(4,4,5,5-tetramethyl-1,3,2-dioxaborolan-2-yl)benzo[d]thiazol-2-yl)carbamate

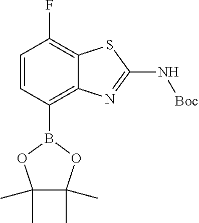 [SMILES: CC(C)(C)OC(=O)Nc1nc2c(B3OC(C)(C)C(C)(C)O3)ccc(F)c2s1]

Synthetic Route

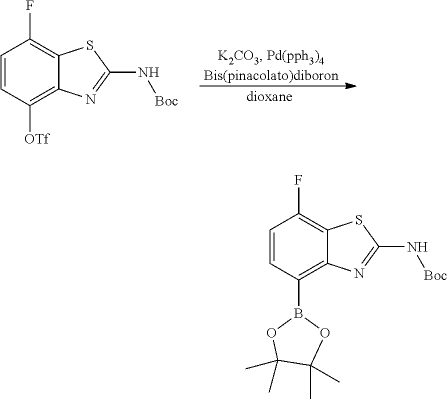 [SMILES: CC(C)(C)OC(=O)Nc1nc2c(OS(=O)(=O)C(F)(F)F)ccc(F)c2s1 ; CC(C)(C)OC(=O)Nc1nc2c(B3OC(C)(C)C(C)(C)O3)ccc(F)c2s1]

A mixture of 2-((tert-butoxycarbonyl)amino)-7-fluorobenzo[d]thiazol-4-yl trifluoromethanesulfonate (30.0 g, 72.1 mmol) and bis(pinacolato)diboron (146 g, 576 mmol), potassium acetate (35.4 g, 360 mmol), Pd(PPh₃)₄ (8.33 g, 7.21 mmol) in dioxane (600 mL) was stirred at 80° C. for 12 hrs. Upon completion, solvents were removed and the residue was partitioned between ethyl acetate (500 mL×3) and 1N aq. HCl (500 mL). The combined organic layer was washed with water, dried over Na₂SO₄ and evaporated to dryness. The residue was purified by column chromatography (SiO₂, Petroleum ether/Ethyl acetate=1/0 to 0/1) to afford the titled product (25.0 g, 29%). ¹H NMR (400 MHz, DMSO-d₆) δ 12.1 (s, 1H), 7.75 (dd, J=6.6, 8.2 Hz, 1H), 7.14 (dd, J=8.4, 9.2 Hz, 1H), 1.50 (s, 9H), 1.31 (s, 12H). MS (ESI, m/e) [M+1]⁺313.0.

### Example 6: 4-(4-(3,6-diazabicyclo[3.1.1]heptan-3-yl)-6-chloro-8-fluoro-2-((1-(piperidin-1-yl)propan-2-yl)oxy)quinazolin-7-yl)-7-fluorobenzo[d]thiazol-2-amine

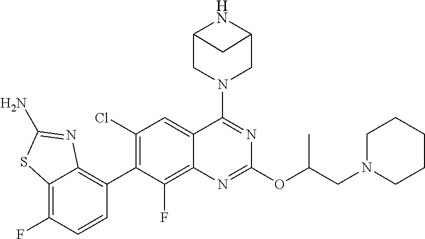 [SMILES: CC(CN1CCCCC1)Oc1nc(N2CC3CC(C2)N3)c2cc(Cl)c(-c3ccc(F)c4sc(N)nc34)c(F)c2n1]

### Step 1: tert-butyl 3-(7-bromo-2,6-dichloro-8-fluoroquinazolin-4-yl)-3,6-diazabicyclo [3.1.1]heptane-6-carboxylate

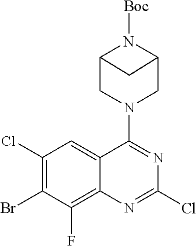 [SMILES: CC(C)(C)OC(=O)N1C2CC1CN(c1nc(Cl)nc3c(F)c(Br)c(Cl)cc13)C2]

To a solution of 7-bromo-2,4,6-trichloro-8-fluoroquinazoline (800 mg, 2.42 mmol) in DCM (48 mL) was added tert-butyl 3,6-diazabicyclo[3.1.1]heptane-6-carboxylate (544 mg, 2.66 mmol) and DIPEA (1.3 mL, 7.26 mmol). The mixture was stirred at room temperature for about 22.5 hrs. The mixture was filtered and the filtrate was concentrated in vacuo to give the crude product (1.18 g, 99%) which was used directly in the next step.

### Step 2: tert-butyl 3-(7-bromo-6-chloro-2,8-difluoroquinazolin-4-yl)-3,6-diazabicyclo[3.1.1]heptane-6-carboxylate

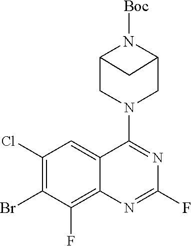 [SMILES: CC(C)(C)OC(=O)N1C2CC1CN(c1nc(F)nc3c(F)c(Br)c(Cl)cc13)C2]

To a solution of tert-butyl 3-(7-bromo-2,6-dichloro-8-fluoroquinazolin-4-yl)-3,6-diazabicyclo[3.1.1] heptane-6-carboxylate (580 mg, 1.18 mmol) in DMSO (24 mL) was added KF (683 mg, 11.8 mmol). The mixture was stirred at 100° C. for 39 hrs. After cooled to room temperature, the mixture was diluted with DCM and H₂O, extracted by DCM. The combined organic layer was washed by H₂O and brine, dried over anhydrous Na₂SO₄, concentrated in vacuo. The residue was purified by chromatography (PE/EtOAc=4:1 to 0:1) to give the product (506 mg, 90%). MS (ESI, m/e) [M+H]⁺ 474.7.

### Step 3: tert-butyl 3-(7-(2-((tert-butoxycarbonyl)amino)-7-fluorobenzo[d]thiazol-4-yl)-6-chloro-2,8-difluoroquinazolin-4-yl)-3,6-diazabicyclo[3.1.1]heptane-6-carboxylate

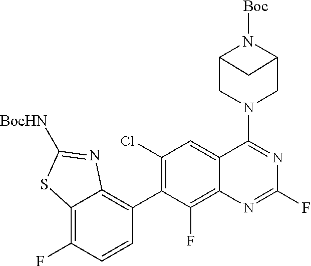 [SMILES: CC(C)(C)OC(=O)Nc1nc2c(-c3c(Cl)cc4c(N5CC6CC(C5)N6C(=O)OC(C)(C)C)nc(F)nc4c3F)ccc(F)c2s1]

To a solution of tert-butyl 3-(7-bromo-6-chloro-2,8-difluoroquinazolin-4-yl)-3,6-diazabicyclo[3.1.1]heptane-6-carboxylate (100 mg, 0.21 mmol) in dioxane/H₂O (10 mL/2 mL) was added tert-butyl (7-fluoro-4-(4,4,5,5-tetramethyl-1,3,2-dioxaborolan-2-yl) benzo[d] thiazol-2-yl)carbamate (100 mg, 0.25 mmol), Pd(dppf)₂Cl₂.DCM (17 mg, 0.021 mmol) and NaHCO₃ (58 mg, 0.69 mmol). The mixture was stirred at 100° C. for 2 hrs. After cooled to room temperature, the mixture was diluted with DCM and H₂O, extracted by DCM. The combined organic layer was washed with brine, dried over anhydrous Na₂SO₄, and concentrated in vacuo. The residue was purified by chromatography (PE/EtOAc=4:1 to 0:1) to give the product (130 mg, 93%). MS (ESI, m/e) [M+H]⁺ 663.3.

### Step 4: tert-butyl 3-(7-(2-((tert-butoxycarbonyl)amino)-7-fluorobenzo[d]thiazol-4-yl)-6-chloro-8-fluoro-2-((1-(piperidin-1-yl)propan-2-yl)oxy)quinazolin-4-yl)-3,6-diazabicyclo[3.1.1] heptane-6-carboxylate

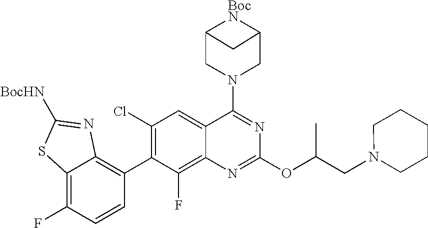 [SMILES: CC(CN1CCCCC1)Oc1nc(N2CC3CC(C2)N3C(=O)OC(C)(C)C)c2cc(Cl)c(-c3ccc(F)c4sc(NC(=O)OC(C)(C)C)nc34)c(F)c2n1]

To a solution of 1-(piperidin-1-yl)propan-2-ol (140 mg, 0.982 mmol) in THF (5 mL) was added TBAI (7 mg, 0.02 mmol) and NaH (47 mg, 1.176 mmol). The mixture was stirred at room temperature for about 3.5 h. Then, tert-butyl 3-(7-(2-((tert-butoxycarbonyl)amino)-7-fluorobenzo[d]thiazol-4-yl)-6-chloro-2,8-difluoroquinazolin-4-yl)-3,6-diazabicyclo[3.1.1]heptane-6-carboxylate (130 mg, 0.196 mmol) in THF (5 mL) was added. The mixture was stirred at 40° C. for 2 h and quenched by saturated NH₄Cl aqueous solution, extracted by ethyl acetate. The organic layers were washed by brine, dried over anhydrous Na₂SO₄, evaporated in vacuo. The residue was purified by chromatography (PE/EtOAc=2:1 to 0:1, then DCM/MeOH=1:0 to 4:1) to give the product (77 mg, 50%). MS (ESI, m/e) [M+H]⁺786.4.

### Step 5: 4-(4-(3,6-diazabicyclo[3.1.1]heptan-3-yl)-6-chloro-8-fluoro-2-((1-(piperidin-1-yl)propan-2-yl)oxy)quinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-6-chloro-8-fluoro-2-((1-(piperidin-1-yl)propan-2-yl)oxy)quinazolin-4-yl)-3,6-diazabicyclo[3.1.1]heptane-6-carboxylate (77 mg, 0.098 mmol) in DCM (20 mL) was added 4 M HCl in dioxane (3.5 mL). The mixture was stirred at room temperature for overnight. Then, the mixture was evaporated in vacuo. The residue was purified by Prep-HPLC to give the product (26.34 mg, 42%). ¹H NMR (500 MHz, CD₃OD) δ 8.48 (s, 1H), 8.40 (s, 1H), 7.30-7.15 (m, 1H), 7.10-6.82 (m, 1H), 5.80-5.45 (m, 1H), 4.64-4.49 (m, 2H), 4.47-4.35 (m, 2H), 4.25-4.05 (m, 2H), 3.30-3.05 (m, 6H), 3.0-2.80 (m, 1H), 1.90-1.70 (m, 5H), 1.70-1.53 (m, 2H), 1.47 (dd, J=6.2, 2.6 Hz, 3H). MS (ESI, m/e) [M+H]⁺ 586.0.

### Example 7: 4-(4-(3,8-diazabicyclo[3.2.1]octan-3-yl)-6-chloro-8-fluoro-2-((tetrahydro-1H-pyrrolizin-7a(5H)-yl)methoxy)quinazolin-7-yl)-7-fluorobenzo[d]thiazol-2-amine

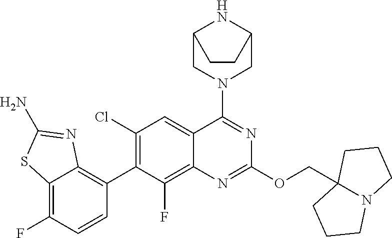 [SMILES: Nc1nc2c(-c3c(Cl)cc4c(N5CC6CCC(C5)N6)nc(OCC56CCCN5CCC6)nc4c3F)ccc(F)c2s1]

### Step 1: tert-butyl-3-(7-(2-((tert-butoxycarbonyl)amino)-7-fluorobenzo[d]thiazol-4-yl)-6-chloro-8-fluoro-2-((tetrahydro-1H-pyrrolizin-7a(5H)-yl)methoxy)quinazolin-4-yl)-3,8-diazabicyclo[3.2.1]octane-8-carboxylate

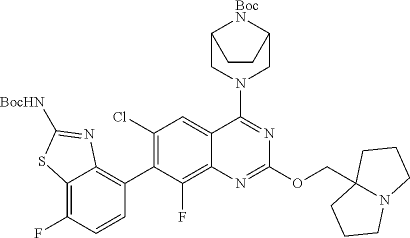 [SMILES: CC(C)(C)OC(=O)Nc1nc2c(-c3c(Cl)cc4c(N5CC6CCC(C5)N6C(=O)OC(C)(C)C)nc(OCC56CCCN5CCC6)nc4c3F)ccc(F)c2s1]

To a solution of (tetrahydro-1H-pyrrolizin-7a(5H)-yl)methanol hydrogen chloride (177 mg, 1.0 mmol) in water (5 mL) was added potassium carbonate (276 mg, 2.0 mmol). The mixture was stirred at room temperature for 30 min. Then it was diluted with a mixture of dichloromethane:methanol=10:1 solvent (5 mL). The organic layer was combined, dried over sodium sulfate and evaporated to give (tetrahydro-1H-pyrrolizin-7a(5H)-yl)methanol (23 mg). To a solution of (tetrahydro-1H-pyrrolizin-7a(5H)-yl)methanol (23 mg, 0.16 mmol) in tetrahydrofuran (3 mL) was added sodium hydride (60%) (12 mg, 0.30 mmol) at 0° C. The mixture was stirred at room temperature for 1 h. Then a solution of tert-butyl 3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (68 mg, 0.10 mmol) in 1 mL tetrahydrofuran was added dropwise and it was stirred at room temperature for another 1 h. The mixture was diluted with dichloromethane and water. Combined organic layer was dried over sodium sulfate and evaporated. The residue was purified by preparative thin layer chromatography to give the title product (20 mg, 25%). MS (ESI, m/e) [M+1]⁺798.4.

### Step 2: 4-(4-(3,8-diazabicyclo[3.2.1]octan-3-yl)-6-chloro-8-fluoro-2-((tetrahydro-1H-pyrrolizin-7a(5H)-yl)methoxy)quinazolin-7-yl)-7-fluorobenzo[d]thiazol-2-amine

To a solution of tert-butyl-3-(7-(2-((tert-butoxycarbonyl)amino)-7-fluorobenzo[d]thiazol-4-yl)-6-chloro-8-fluoro-2-((tetrahydro-1H-pyrrolizin-7a(5H)-yl)methoxy)quinazolin-4-yl)-3,8-diazabicyclo[3.2.1]octane-8-carboxylate (20 mg, 0.025 mmol) in dichloromethane (1.5 mL) was added trifluoroacetic acid (1 mL) and it was stirred at room temperature for 2 h. Solvent was evaporated and the residue was adjusted to pH=11 with aq potassium carbonate. The mixture was diluted with dichloromethane:methanol=10:1 solvent and water. Combined organic layer was dried over sodium sulfate and evaporated. The residue was lyophilized to give the title product (7 mg, 46.7%). ¹H NMR (500 MHz, CD₃OD) δ 7.88 (s, 1H), 7.24-7.17 (m, 1H), 7.02-6.94 (m, 1H), 4.55-4.52 (m, 1H), 4.45-4.42 (m, 1H), 4.32 (s, 2H), 3.65-3.57 (m, 4H), 3.32-3.20 (m, 2H), 2.88-2.77 (m, 2H), 2.15-2.05 (m, 2H), 2.03-1.87 (m, 4H), 1.86-1.71 (m, 6H). MS (ESI, m/e) [M+H]⁺ 598.4.

### Example 8: 4-(4-(3,8-diazabicyclo[3.2.1]octan-8-yl)-6-chloro-8-fluoro-2-((1-(piperidin-1-yl)propan-2-yl)oxy)quinazolin-7-yl)-7-fluorobenzo[d]thiazol-2-amine

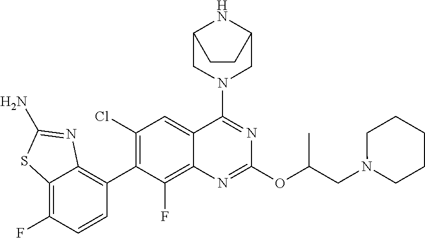 [SMILES: CC(CN1CCCCC1)Oc1nc(N2CC3CCC(C2)N3)c2cc(Cl)c(-c3ccc(F)c4sc(N)nc34)c(F)c2n1]

### Step 1: tert-butyl 8-(7-bromo-2,6-dichloro-8-fluoroquinazolin-4-yl)-3,8-diazabicyclo [3.2.1]octane-3-carboxylate

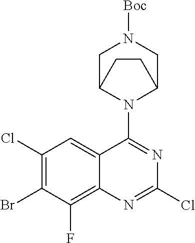 [SMILES: CC(C)(C)OC(=O)N1CC2CCC(C1)N2c1nc(Cl)nc2c(F)c(Br)c(Cl)cc12]

To a solution of 7-bromo-2,4,6-trichloro-8-fluoroquinazoline (200 mg, 0.605 mmol) in DCM (12 mL) was added tert-butyl 3,8-diazabicyclo[3.2.1]octane-3-carboxylate (146 mg, 0.666 mmol) and DIPEA (0.3 mL, 1.815 mmol). The mixture was stirred at room temperature for about 26.5 hrs. The mixture was filtered and the filtrate was concentrated in vacuo to give the crude product (280 mg, 91%) which was used directly in the next step.

### Step 2: tert-butyl 8-(7-bromo-6-chloro-2,8-difluoroquinazolin-4-yl)-3,8-diazabicyclo [3.2.1]octane-3-carboxylate

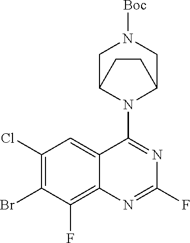 [SMILES: CC(C)(C)OC(=O)N1CC2CCC(C1)N2c1nc(F)nc2c(F)c(Br)c(Cl)cc12]

To a solution of tert-butyl 8-(7-bromo-2,6-dichloro-8-fluoroquinazolin-4-yl)-3,8-diaza bicyclo[3.2.1]octane-3-carboxylate (280 mg, 0.553 mmol) in DMSO (11 mL) was added KF (32 mg, 5.53 mmol). The mixture was stirred at 100° C. for 20 hrs. After cooled to room temperature, the mixture was diluted with DCM and H₂O. The organic layer was washed by H₂O and brine, dried over anhydrous Na₂SO₄, concentrated in vacuo. The residue was purified by chromatography (PE/EtOAc=20:1 to 0:1) to give the product (110 mg, 41%). MS (ESI, m/e) [M+H]⁺ 489.3.

### Step 3: tert-butyl 8-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-3-carboxylate

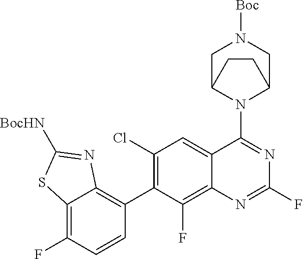 [SMILES: CC(C)(C)OC(=O)Nc1nc2c(-c3c(Cl)cc4c(N5C6CCC5CN(C(=O)OC(C)(C)C)C6)nc(F)nc4c3F)ccc(F)c2s1]

To a solution of tert-butyl 8-(7-bromo-6-chloro-2,8-difluoroquinazolin-4-yl)-3,8-diaza bicyclo[3.2.1]octane-3-carboxylate (200 mg, 0.408 mmol) in dioxane/H₂O (10 mL/2 mL) was added tert-butyl (7-fluoro-4-(4,4,5,5-tetramethyl-1,3,2-dioxaborolan-2-yl) benzo[d] thiazol-2-yl)carbamate (194 mg, 0.49 mmol), Pd(dppf)₂Cl₂.DCM (34 mg, 0.0408 mmol) and NaHCO₃ (114 mg, 1.36 mmol). The mixture was stirred at 100° C. for 2 hrs. After cooled to room temperature, the mixture was diluted with DCM and H₂O, extracted by DCM. The organic layer was washed by brine, dried over anhydrous Na₂SO₄, and concentrated in vacuo. The residue was purified by chromatography (PE/EtOAc=8:1 to 0:1) to give the product (234 mg, 85%). MS (ESI, m/e) [M+H]⁺ 677.3.

### Step 4: tert-butyl 8-(7-(2-((tert-butoxycarbonyl)amino)-7-fluorobenzo[d]thiazol-4-yl)-6-chloro-8-fluoro-2-((1-(piperidin-1-yl)propan-2-yl)oxy)quinazolin-4-yl)-3,8-diazabicyclo[3.2.1] octane-3-carboxylate

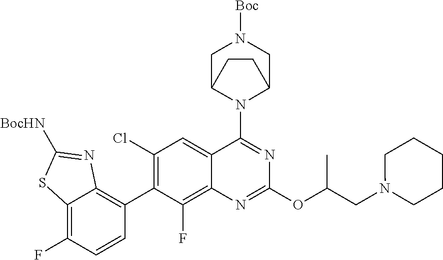 [SMILES: CC(CN1CCCCC1)Oc1nc(N2C3CCC2CN(C(=O)OC(C)(C)C)C3)c2cc(Cl)c(-c3ccc(F)c4sc(NC(=O)OC(C)(C)C)nc34)c(F)c2n1]

To a solution of 1-(piperidin-1-yl)propan-2-ol (106 mg, 0.74 mmol) in THF (7.5 mL) was added TBAI (5 mg, 0.015 mmol) and NaH (36 mg, 0.888 mmol) at room temperature. The mixture was stirred at room temperature for about 3.5 h. Then, tert-butyl 8-(7-(2-((tert-butoxycarbonyl)amino)-7-fluorobenzo[d]thiazol-4-yl)-6-chloro-2,8-difluoro quinazolin-4-yl)-3,8-diazabicyclo[3.2.1]octane-3-carboxylate (120 mg, 0.178 mmol) in THF (5 mL) was added to the mixture. The mixture was stirred at 40° C. for 3 hrs, quenched with saturated NH₄Cl aqueous solution, and extracted by ethyl acetate. The organic layers were washed by brine, dried over anhydrous Na₂SO₄, and evaporated in vacuo. The residue was purified by chromatography (PE/EtOAc=2:1 to 0:1, then DCM/MeOH=1:0 to 4:1) to give the product (121 mg, 92%). MS (ESI, m/e) [M+H]⁺ 800.4.

### Step 5: 4-(4-(3,8-diazabicyclo[3.2.1]octan-8-yl)-6-chloro-8-fluoro-2-((1-(piperidin-1-yl) propan-2-yl)oxy)quinazolin-7-yl)-7-fluorobenzo[d]thiazol-2-amine

To a solution of tert-butyl 8-(7-(2-((tert-butoxycarbonyl)amino)-7-fluorobenzo[d]thiazol-4-yl)-6-chloro-8-fluoro-2-((1-(piperidin-1-yl)propan-2-yl)oxy)quinazolin-4-yl)-3,8-diazabicyclo [3.2.1]octane-3-carboxylate (109 mg, 0.136 mmol) in DCM (10 mL) was added TFA (1 mL). The mixture was stirred at room temperature for overnight. Then, the mixture was evaporated in vacuo. The residue was re-dissolved in MeOH and 7 M NH₃ in MeOH was added to adjust pH >7. Then, the solvent was removed in vacuo and the residue was purified by Prep-HPLC to give the product (27.65 mg, 34%). ¹H NMR (500 MHz, CD₃OD) δ 7.99 (s, 1H), 7.24 (dd, J=8.4, 5.4 Hz, 1H), 7.02 (t, J=8.8 Hz, 1H), 5.74-5.51 (m, 1H), 5.06 (d, J=14.4 Hz, 2H), 3.50 (dd, J=13.6, 9.9 Hz, 4H), 3.38 (d, J=13.7 Hz, 2H), 3.27-3.15 (m, 4H), 2.23 (d, J=5.8 Hz, 2H), 2.12 (d, J=8.4 Hz, 2H), 1.90-1.70 (m, 4H), 1.70-1.50 (m, 2H), 1.49 (d, J=6.3 Hz, 3H). MS (ESI, m/e) [M+H]⁺ 600.3.

### Example 9: 4-(4-(2-amino-7-azabicyclo[2.2.1]heptan-7-yl)-6-chloro-8-fluoro-2-((1-(piperidin-1-yl)propan-2-yl)oxy)quinazolin-7-yl)-7-fluorobenzo[d]thiazol-2-amine

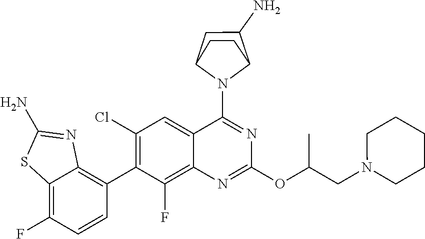 [SMILES: CC(CN1CCCCC1)Oc1nc(N2C3CCC2C(N)C3)c2cc(Cl)c(-c3ccc(F)c4sc(N)nc34)c(F)c2n1]

### Step 1: tert-butyl (7-(7-bromo-2,6-dichloro-8-fluoroquinazolin-4-yl)-7-azabicyclo [2.2.1]heptan-2-yl) carbamate

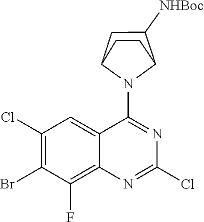 [SMILES: CC(C)(C)OC(=O)NC1CC2CCC1N2c1nc(Cl)nc2c(F)c(Br)c(Cl)cc12]

To a solution of 7-bromo-2,4,6-trichloro-8-fluoroquinazoline (330 mg, 1 mmol) in DCM (40 mL) was added tert-butyl (7-azabicyclo[2.2.1]heptan-2-yl)carbamate (318 mg, 1.5 mmol) and DIPEA (387 mg, 3 mmol), and the mixture was stirred at room temperature for 1 h. The resulting mixture was concentrated and the product was purified by chromatography column on silica (eluting with PE/EtOAc=5/1) to give the title product (130 mg, 26%). MS (ESI, m/e) [M+1]⁺507.0.

### Step 2: tert-butyl (7-(7-bromo-6-chloro-2,8-difluoroquinazolin-4-yl)-7-azabicyclo [2.2.1]heptan-2-yl) carbamate

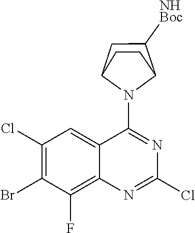 [SMILES: CC(C)(C)OC(=O)NC1CC2CCC1N2c1nc(Cl)nc2c(F)c(Br)c(Cl)cc12]

To a solution of tert-butyl (7-(7-bromo-2,6-dichloro-8-fluoroquinazolin-4-yl)-7-azabicyclo [2.2.1] heptan-2-yl) carbamate (130 mg, 0.257 mmol) in DMSO (10 mL) was added KF (149 mg, 2.57 mmol), and the mixture was stirred at 100° C. for overnight. The resulting mixture was diluted with water (20 mL) and extracted with DCM (30 mL×3), and the combined organic layer was concentrated to give a residue which was purified by chromatography column on silica (eluting with PE/EtOAc=5/1) to give the title product (124 mg, 100%). MS (ESI, m/e) [M+1]⁺491.0.

### Step 3: tert-butyl (7-(7-bromo-6-chloro-8-fluoro-2-((1-(piperidin-1-yl)propan-2-yl)oxy)quinazolin-4-yl)-7-azabicyclo[2.2.1]heptan-2-yl

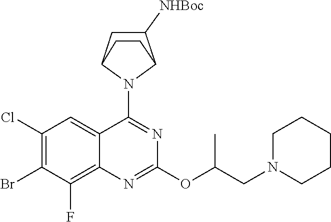 [SMILES: CC(CN1CCCCC1)Oc1nc(N2C3CCC2C(NC(=O)OC(C)(C)C)C3)c2cc(Cl)c(Br)c(F)c2n1]

NaH (51 mg, 1.2/8 mmol) was added to a solution of 1-(piperidin-1-yl) propan-2-ol (104 mg, 0.213 mmol) in THF (5 mL), and the mixture was stirred at 0° C. for 0.5 h. Then tert-butyl (7-(7-bromo-6-chloro-2,8-difluoroquinazolin-4-yl)-7-azabicyclo [2.2.1] heptan-2-yl) carbamate (104 mg, 0.213 mmol) in THF (50 mL) was added and the mixture was stirred at room temperature for 1 h. The resulting mixture was quenched with ice water (5 mL) and extracted with EtOAc (10 mL×3), and the combined organic layer was concentrated to give a residue which was purified by chromatography column on silica (eluting with DCM/MeOH=10/1) to give the title product (115 mg, 88%). MS (ESI, m/e) [M+1]⁺614.1.

### Step 4: tert-butyl (7-(7-(2-((tert-butoxycarbonyl)amino)-7-fluorobenzo[d]thiazol-4-yl)-6-chloro-8-fluoro-2-((1-(piperidin-1-yl)propan-2-yl)oxy)quinazolin-4-yl)-7-azabicyclo[2.2.1]heptan-2-yl)carbamate

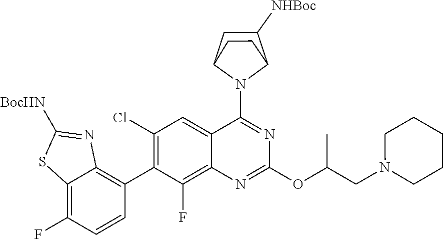 [SMILES: CC(CN1CCCCC1)Oc1nc(N2C3CCC2C(NC(=O)OC(C)(C)C)C3)c2cc(Cl)c(-c3ccc(F)c4sc(NC(=O)OC(C)(C)C)nc34)c(F)c2n1]

To a solution of tert-butyl (7-(7-bromo-6-chloro-8-fluoro-2-((1-(piperidin-1-yl)propan-2-yl)oxy)quinazolin-4-yl)-7-azabicyclo[2.2.1]heptan-2-yl)carbamate (100 mg, 0.164 mmol) in dioxane (15 mL) was added tert-butyl (7-fluoro-4-(4,4,5,5-tetramethyl-1,3,2-dioxaborolan-2-yl)benzo[d]thiazol-2-yl)carbamate (104 mg, 0.262 mmol), Pd(dppf)Cl₂ (13.5 mg, 0.0164 mmol), NaHCO₃ (41 mg, 0.492 mmol) and water (3 mL), and the mixture was stirred at 100° C. for 2 h. The resulting mixture was concentrated and the crude product was purified by chromatography column on silica (eluting with DCM/MeOH=20/1) to give the crude product which was used in the next step directly. MS (ESI, m/e) [M+1]⁺800.3.

### Step 5: 4-(4-(2-amino-7-azabicyclo[2.2.1]heptan-7-yl)-6-chloro-8-fluoro-2-((1-(piperidin-1-yl)propan-2-yl)oxy)quinazolin-7-yl)-7-fluorobenzo[d]thiazol-2-amine

To a solution of tert-butyl (7-(7-(2-((tert-butoxycarbonyl)amino)-7-fluorobenzo[d]thiazol-4-yl)-6-chloro-8-fluoro-2-((1-(piperidin-1-yl)propan-2-yl)oxy)quinazolin-4-yl)-7-azabicyclo[2.2.1]heptan-2-yl)carbamate (20 mg, 0.025 mmol) in DCM (6 mL) was added TFA (1.5 mL), the mixture was stirred at room temperature for 1 h. The resulting mixture was concentrated and pH was adjusted to 7 with Na₂CO₃. Organic layer was concentrated to give a residue which was further purified by Prep-HPLC to give the title product (8.1 mg, 53%). ¹H NMR (500 MHz, CD₃OD) δ 8.14-7.97 (m, 1H), 7.25-7.18 (m, 1H), 7.00 (m, 2.5 Hz, 1H), 5.69 (m, 1H), 5.03-4.90 (m, 2H), 3.39 (m, 1H), 3.21-3.11 (m, 2H), 2.04 (m, 3H), 1.79 (m, 6H), 1.70-1.54 (m, 4H), 1.46 (m, 4H), 1.30 (m, 2H). MS (ESI, m/e) [M+1]⁺600.2.

### Example 10: 4-(2-((1-(3,8-diazabicyclo[3.2.1]octan-8-yl)propan-2-yl)oxy)-4-(3,8-diaza bicyclo[3.2.1]octan-3-yl)-6-chloro-8-fluoroquinazolin-7-yl)-7-fluorobenzo[d]thiazol-2-amine

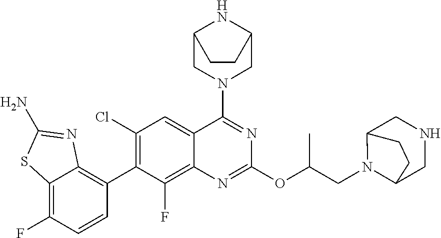 [SMILES: CC(CN1C2CCC1CNC2)Oc1nc(N2CC3CCC(C2)N3)c2cc(Cl)c(-c3ccc(F)c4sc(N)nc34)c(F)c2n1]

### Step 1: tert-butyl 8-(2-hydroxypropyl)-3,8-diazabicyclo[3.2.1]octane-3-carboxylate

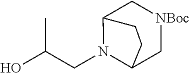 [SMILES: CC(O)CN1C2CCC1CN(C(=O)OC(C)(C)C)C2]

To a solution of tert-butyl 3,8-diazabicyclo[3.2.1]octane-3-carboxylate (500 mg, 2.36 mmol) in H₂O (24 mL) was added 2-methyloxirane (0.25 mL, 3.53 mmol). The mixture was stirred at room temperature for about 17 h. The mixture was extracted by ethyl acetate, washed by brine, dried over anhydrous Na₂SO₄ and concentrated in vacuo to give the crude product which was used directly in the next step. MS (ESI, m/e) [M+H]⁺ 271.2.

### Step 2: tert-butyl 8-(2-((4-(8-(tert-butoxycarbonyl)-3,8-diazabicyclo[3.2.1]octan-3-yl)-7-(2-((tert-butoxycarbonyl)amino)-7-fluorobenzo[d]thiazol-4-yl)-6-chloro-8-fluoroquinazolin-2-yl)oxy)propyl)-3,8-diazabicyclo[3.2.1]octane-3-carboxylate

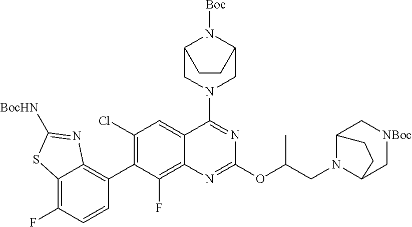 [SMILES: CC(CN1C2CCC1CN(C(=O)OC(C)(C)C)C2)Oc1nc(N2CC3CCC(C2)N3C(=O)OC(C)(C)C)c2cc(Cl)c(-c3ccc(F)c4sc(NC(=O)OC(C)(C)C)nc34)c(F)c2n1]

To a solution of tert-butyl 8-(2-hydroxypropyl)-3,8-diazabicyclo[3.2.1]octane-3-carboxylate (120 mg, 0.444 mmol) in THF (7.5 mL) was added TBAI (6 mg, 0.0148 mmol) and NaH (24 mg, 0.592 mmol). The mixture was stirred at room temperature for about 20 mins. Then, tert-butyl 3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1] octane-8-carboxylate (100 mg, 0.148 mmol) in THF (7.5 mL) was added to the mixture. The mixture was stirred at 40° C. for about 2 hrs and quenched by saturated NH₄Cl aqueous solution, extracted by ethyl acetate. The organic layers were washed by brine, dried over anhydrous Na₂SO₄ and evaporated in vacuo. The residue was purified by chromatography (PE/EtOAc=2:1 to 0:1, then DCM/MeOH=1:0 to 4:1) to give the crude product (112 mg, 81%).

### Step 3: 4-(2-((1-(3,8-diazabicyclo[3.2.1]octan-8-yl)propan-2-yl)oxy)-4-(3,8-diazabicyclo [3.2.1]octan-3-yl)-6-chloro-8-fluoroquinazolin-7-yl)-7-fluorobenzo[d]thiazol-2-amine

To a solution of tert-butyl 8-(2-((4-(8-(tert-butoxycarbonyl)-3,8-diazabicyclo[3.2.1]octan-3-yl)-7-(2-((tert-butoxycarbonyl)amino)-7-fluorobenzo[d]thiazol-4-yl)-6-chloro-8-fluoroquinazolin-2-yl)oxy)propyl)-3,8-diazabicyclo[3.2.1]octane-3-carboxylate (112 mg, 0.12 mmol) in DCM (10 mL) was added TFA (2 mL). The mixture was stirred at room temperature for overnight. Then, the mixture was evaporated in vacuo. The residue was re-dissolved in MeOH and 7 M NH₃ in MeOH was added to adjust pH >7. Then, the solvent was removed in vacuo and the residue was purified by Prep-HPLC to give the product (17 mg, 20%). ¹H NMR (500 MHz, CD₃OD) δ 8.47 (s, 1H), 7.88 (s, 1H), 7.21 (dd, J=8.3, 5.4 Hz, 1H), 7.00 (t, J=8.8 Hz, 1H), 5.52-5.33 (m, 1H), 4.54 (dd, J=22.7, 14.2 Hz, 2H), 4.13-3.92 (m, 2H), 3.74 (t, J=13.7 Hz, 2H), 3.67-3.61 (m, 1H), 3.61-3.48 (m, 1H), 3.09 (d, J=11.3 Hz, 2H), 3.01 (d, J=12.2 Hz, 2H), 2.80 (dd, J=13.1, 7.0 Hz, 1H), 2.60 (d, J=13.0 Hz, 1H), 2.26-2.10 (m, 2H), 2.08-1.97 (m, 4H), 1.83 (d, J=8.7 Hz, 2H), 1.45 (d, J=6.2 Hz, 3H). MS (ESI, m/e) [M+H]⁺ 627.4.

### Example 11: 4-(4-(3,8-diazabicyclo[3.2.1]octan-3-yl)-6-chloro-8-fluoro-2-(((S)-1-(piperidin-1-yl)propan-2-yl)oxy)quinazolin-7-yl)-7-fluorobenzo[d]thiazol-2-amine

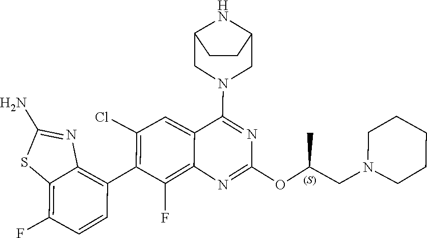

### Step 1: tert-butyl 3-(7-(2-((tert-butoxycarbonyl)amino)-7-fluorobenzo[d]thiazol-4-yl)-6-chloro-8-fluoro-2-(((S)-1-(piperidin-1-yl)propan-2-yl)oxy)quinazolin-4-yl)-3,8-diazabicyclo[3.2.1]octane-8-carboxylate

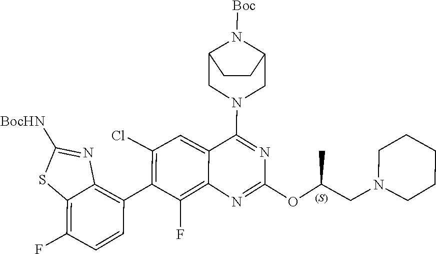

To a solution of (S)-1-(piperidin-1-yl)propan-2-ol (63 mg, 0.444 mmol) in THF (7.5 mL) was added TBAI (6 mg, 0.0148 mmol) and NaH (24 mg, 0.592 mmol). The mixture was stirred at room temperature for about 20 mins. Then, tert-butyl 3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (100 mg, 0.148 mmol) in THF (7.5 mL) was added to the mixture. The mixture was stirred at 40° C. for about 2 hrs and quenched by saturated NH₄Cl aqueous solution, extracted by ethyl acetate. The organic layers were washed by brine, dried over anhydrous Na₂SO₄ and evaporated in vacuo. The residue was purified by chromatography (PE/EtOAc=2:1 to 0:1, then DCM/MeOH=1:0 to 4:1) to give the crude product which was used directly in the next step.

### Step 2: 4-(4-(3,8-diazabicyclo[3.2.1]octan-3-yl)-6-chloro-8-fluoro-2-(((S)-1-(piperidin-1-yl)propan-2-yl)oxy)quinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-6-chloro-8-fluoro-2-(((S)-1-(piperidin-1-yl)propan-2-yl)oxy)quinazolin-4-yl)-3,8-diazabicyclo[3.2.1] octane-8-carboxylate (150 mg, 0.188 mmol) in DCM (10 mL) was added TFA (2 mL). The mixture was stirred at room temperature for overnight. Then, the mixture was evaporated in vacuo. The residue was re-dissolved in MeOH and 7 M NH₃ in MeOH was added to adjust pH >7. The solvent was removed in vacuo and the residue was purified by Prep-HPLC to give the product (2.58 mg, 2%). ¹H NMR (500 MHz, d4-MeOH) δ 8.48 (s, 2H), 7.93 (s, 1H), 7.23 (dd, J=8.4, 5.4 Hz, 1H), 7.01 (t, J=8.8 Hz, 1H), 5.76-5.58 (m, 1H), 4.60 (dd, J=29.2, 13.4 Hz, 2H), 4.21-3.96 (m, 2H), 3.93-3.71 (m, 2H), 3.47 (dd, J=13.8, 9.5 Hz, 2H), 3.38-3.31 (m, 2H), 3.30-3.09 (m, 2H), 2.06 (t, J=9.7 Hz, 4H), 1.92-1.73 (m, 4H), 1.70-1.55 (m, 2H), 1.48 (d, J=6.2 Hz, 3H). MS (ESI, m/e) [M+H]⁺ 600.3.

### Example 12: 4-(4-(3,6-diazabicyclo[3.1.1]heptan-3-yl)-6-chloro-8-fluoro-2-(((S)-1-(piperidin-1-yl)propan-2-yloxy)quinazolin-7-yl)-7-fluorobenzo[d]thiazol-2-amine

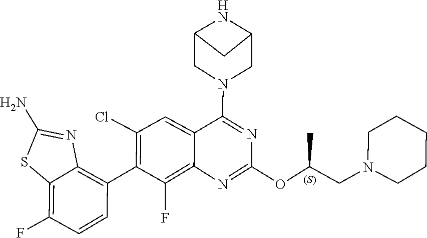

### Step 1: tert-butyl 3-(7-(2-((tert-butoxycarbonyl)amino)-7-fluorobenzo[d]thiazol-4-yl)-6-chloro-8-fluoro-2-(((S)-1-(piperidin-1-yl)propan-2-yl)oxy)quinazolin-4-yl)-3,6-diazabicyclo [3.1.1]heptane-6-carboxylate

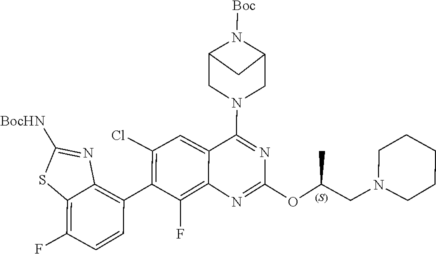

To a solution of (S)-1-(piperidin-1-yl)propan-2-ol (64 mg, 0.448 mmol) in THF (7.5 mL) was added TBAI (6 mg, 0.0148 mmol) and NaH (24 mg, 0.592 mmol). The mixture was stirred at room temperature for about 20 mins. Then, tert-butyl 3-(7-(2-((tert-butoxycarbonyl)amino)-7-fluorobenzo[d]thiazol-4-yl)-6-chloro-2,8-difluoroquinazolin-4-yl)-3,6-diazabicyclo[3.1.1]heptane-6-carboxylate (100 mg, 0.151 mmol) in THF (7.5 mL) was added to the mixture. The mixture was stirred at 40° C. for about 1.5 hrs and quenched by saturated NH₄Cl aqueous solution, extracted by ethyl acetate. The organic layers were washed by brine, dried over anhydrous Na₂SO₄ and evaporated in vacuo. The residue was purified by chromatography (PE/EtOAc=2:1 to 0:1, then DCM/MeOH=1:0 to 4:1) to give the crude product which was used directly in the next step.

### Step 2: 4-(4-(3,6-diazabicyclo[3.1.1]heptan-3-yl)-6-chloro-8-fluoro-2-(((S)-1-(piperidin-1-yl)propan-2-yl)oxy)quinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-6-chloro-8-fluoro-2-(((S)-1-(piperidin-1-yl)propan-2-yl)oxy)quinazolin-4-yl)-3,6-diazabicyclo[3.1.1]heptane-6-carboxylate (88 mg, 0.113 mmol) in DCM (10 mL) was added TFA (2 mL). The mixture was stirred at room temperature for overnight. Then, the mixture was evaporated in vacuo. The residue was re-dissolved in MeOH and 7 M NH₃ in MeOH was added to adjust pH >7. The solvent was removed in vacuo and the residue was purified by Prep-HPLC to give the product (19.84 mg, 28%). ¹H NMR (500 MHz, CD₃OD) δ 8.53 (s, 1H), 8.41 (s, 1H), 7.23 (dd, J=8.4, 5.4 Hz, 1H), 7.00 (t, J=8.8 Hz, 1H), 5.69-5.53 (m, 1H), 4.47 (d, J=12.5 Hz, 2H), 4.37 (d, J=12.4, 2H), 4.11-3.89 (m, 2H), 3.30-3.17 (m, 2H), 3.17-2.91 (m, 4H), 2.86-2.78 (m, 1H), 1.74 (dd, J=11.6, 7.3 Hz, 5H), 1.64-1.50 (m, 2H), 1.45 (d, J=6.3 Hz, 3H). MS (ESI, m/e) [M+H]⁺ 586.3.

### Example 13: 4-(4-(3,8-diazabicyclo[3.2.1]octan-3-yl)-6-chloro-8-fluoro-2-((1-(piperazin-1-yl)propan-2-yl)oxy)quinazolin-7-yl)-7-fluorobenzo[d]thiazol-2-amine

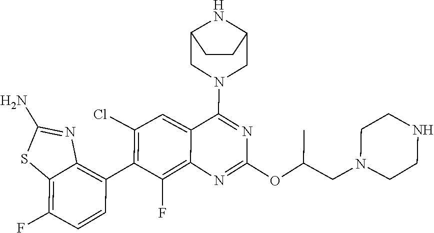 [SMILES: CC(CN1CCNCC1)Oc1nc(N2CC3CCC(C2)N3)c2cc(Cl)c(-c3ccc(F)c4sc(N)nc34)c(F)c2n1]

### Step 1: tert-butyl 3-(7-(2-((tert-butoxycarbonyl)amino)-7-fluorobenzo[d]thiazol-4-yl)-2-((1-(4-(tert-butoxycarbonyl)piperazin-1-yl)propan-2-yl)oxy)-6-chloro-8-fluoroquinazolin-4-yl)-3,8-diazabicyclo[3.2.1]octane-8-carboxylate

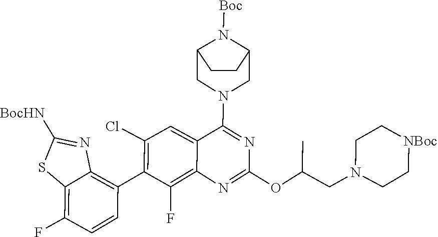 [SMILES: CC(CN1CCN(C(=O)OC(C)(C)C)CC1)Oc1nc(N2CC3CCC(C2)N3C(=O)OC(C)(C)C)c2cc(Cl)c(-c3ccc(F)c4sc(NC(=O)OC(C)(C)C)nc34)c(F)c2n1]

To a solution of tert-butyl 4-(2-hydroxypropyl)piperazine-1-carboxylate (108 mg, 0.443 mmol) in THF (7.5 mL) was added TBAI (5 mg, 0.0135 mmol) and NaH (24 mg, 0.592 mmol). The mixture was stirred at room temperature for about 10 mins. Then, tert-butyl 3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (113 mg, 0.167 mmol) in THF (7.5 mL) was added to the mixture. The mixture was stirred at 40° C. for about 1 h and quenched by saturated NH₄Cl aqueous solution, extracted by ethyl acetate. The organic layers were washed by brine, dried over anhydrous Na₂SO₄ and evaporated in vacuo. The residue was purified by chromatography (PE/EtOAc=2:1 to 0:1, then DCM/MeOH=1:0 to 4:1) to give the title product (141 mg, 94%).

### Step 2: 4-(4-(3,8-diazabicyclo[3.2.1]octan-3-yl)-6-chloro-8-fluoro-2-((1-(piperazin-1-yl)propan-2-yl)oxy)quinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-2-((1-(4-(tert-butoxycarbonyl)piperazin-1-yl)propan-2-yl)oxy)-6-chloro-8-fluoroquinazolin-4-yl)-3,8-diazabicyclo[3.2.1]octane-8-carboxylate (100 mg, 0.111 mmol) in DCM (10 mL) was added TFA (2 mL). The mixture was stirred at room temperature for overnight. Then, the mixture was evaporated in vacuo. The residue was re-dissolved in MeOH and 7 M NH₃ in MeOH was added to adjust pH >7. Then, the solvent was removed in vacuo and the residue was purified by Prep-HPLC to give the product (11.35 mg, 15%). ¹H NMR (500 MHz, CD₃OD) δ 8.41 (s, 2H), 7.88 (s, 1H), 7.21 (dd, J=8.4, 5.5 Hz, 1H), 7.00 (t, J=8.8 Hz, 1H), 5.60-5.47 (m, 1H), 4.53 (t, J=11.7 Hz, 2H), 4.16-4.00 (m, 2H), 3.84-3.68 (m, 2H), 3.19-3.07 (m, 4H), 2.94-2.69 (m, 5H), 2.62 (dd, J=13.4, 4.3 Hz, 1H), 2.17-1.99 (m, 4H), 1.41 (d, J=6.3 Hz, 3H). MS (ESI, m/e) [M+H]⁺ 601.3.

### Example 14: 4-(4-(3,8-diazabicyclo[3.2.1]octan-3-yl)-6-chloro-8-fluoro-2-(((2S,4R)-4-methoxy-1-methylpyrrolidin-2-yl)methoxy)quinazolin-7-yl)-7-fluorobenzo[d]thiazol-2-amine

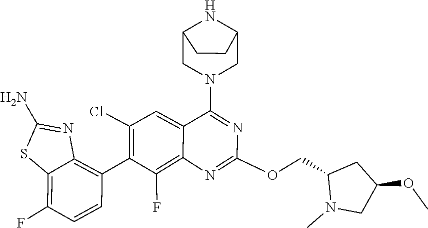 [SMILES: CO[C@@H]1C[C@@H](COc2nc(N3CC4CCC(C3)N4)c3cc(Cl)c(-c4ccc(F)c5sc(N)nc45)c(F)c3n2)N(C)C1]

### Step 1: tert-butyl 3-(7-(2-((tert-butoxycarbonyl)amino)-7-fluorobenzo[d]thiazol-4-yl)-6-chloro-8-fluoro-2-(((2S,4R)-4-methoxy-1-methylpyrrolidin-2-yl)methoxy)quinazolin-4-yl)-3,8-diazabicyclo[3.2.1]octane-8-carboxylate

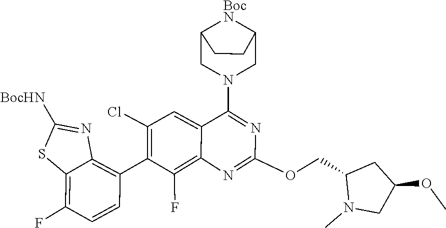 [SMILES: CO[C@@H]1C[C@@H](COc2nc(N3CC4CCC(C3)N4C(=O)OC(C)(C)C)c3cc(Cl)c(-c4ccc(F)c5sc(NC(=O)OC(C)(C)C)nc45)c(F)c3n2)N(C)C1]

To a solution of ((2S,4R)-4-methoxy-1-methylpyrrolidin-2-yl)methanol (60 mg, 0.41 mmol) in tetrahydrofuran (5 mL) was added sodium hydride (60%) (24 mg, 0.60 mmol) at 0° C. The mixture was stirred at room temperature for 1 h. Then tert-butyl-3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (67 mg, 0.10 mmol) in tetrahydrofuran (1 mL) was added dropwise and it was stirred at room temperature for another 1 h. The mixture was diluted with dichloromethane and water. The organic layer was combined, dried over sodium sulfate and evaporated. The residue was purified by preparative thin layer chromatography to give the title product (40 mg, 49.8%). MS (ESI, m/e) [M+1]⁺802.4.

### Step 2: 4-(4-(3,8-diazabicyclo[3.2.1]octan-3-yl)-6-chloro-8-fluoro-2-(((2S,4R)-4-methoxy-1-methylpyrrolidin-2-yl)methoxy)quinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-6-chloro-8-fluoro-2-(((2S,4R)-4-methoxy-1-methylpyrrolidin-2-yl)methoxy)quinazolin-4-yl)-3,8-diazabicyclo[3.2.1]octane-8-carboxylate (40 mg, 0.05 mmol) in dichloromethane (2 mL) was added trifluoroacetic acid (1.5 mL) and it was stirred at room temperature for 3 hrs. Solvent was evaporated and the residue was adjusted to pH=11 with aq. Potassium carbonate. The mixture was diluted with dichloromethane:methanol=10:1 solvent and water. Combined organic layer was dried over sodium sulfate and evaporated. The residue was lyophilized to give the title product (20 mg, 66.6%). ¹H NMR (500 MHz, CD₃OD) δ 7.88-7.85 (m, 1H), 7.24-7.18 (m, 1H), 7.01-6.94 (m, 1H), 4.54-4.36 (m, 4H), 4.00-3.94 (m, 1H), 3.69-3.53 (m, 5H), 3.43-3.36 (m, 1H), 3.26-3.18 (m, 1H), 3.00-2.94 (m, 1H), 2.83-2.74 (m, 1H), 2.40-2.30 (m, 2H), 2.12-2.04 (m, 1H), 1.98=1.88 (m, 1H), 1.86-1.73 (m, 6H). MS (ESI, m/e) [M+H]⁺ 602.4.

### Example 15: 4-(4-(3,8-diazabicyclo[3.2.1]octan-3-yl)-6-chloro-8-fluoro-2-(((2S,4R)-4-fluoro-1-methylpyrrolidin-2-yl)methoxy)quinazolin-7-yl)-7-fluorobenzo[d]thiazol-2-amine

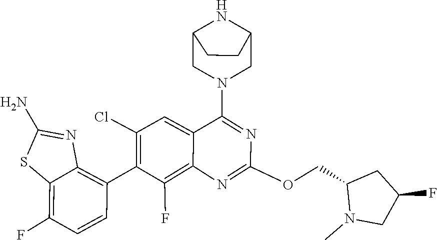 [SMILES: CN1C[C@H](F)C[C@H]1COc1nc(N2CC3CCC(C2)N3)c2cc(Cl)c(-c3ccc(F)c4sc(N)nc34)c(F)c2n1]

### Step 1: tert-butyl (3-(7-(2-((tert-butoxycarbonyl)amino)-7-fluorobenzo[d]thiazol-4-yl)-6-chloro-8-fluoro-2-(((2S,4R)-4-fluoro-1-methylpyrrolidin-2-yl)methoxy)quinazolin-4-yl)-3,8-diazabicyclo[3.2.1]octane-8-carboxylate

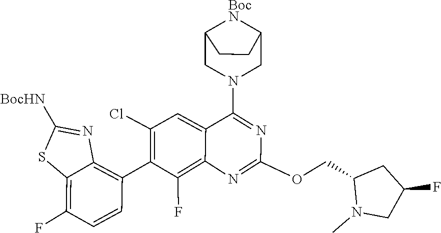 [SMILES: CN1C[C@H](F)C[C@H]1COc1nc(N2CC3CCC(C2)N3C(=O)OC(C)(C)C)c2cc(Cl)c(-c3ccc(F)c4sc(NC(=O)OC(C)(C)C)nc34)c(F)c2n1]

To a solution of ((2S,4R)-4-fluoro-1-methylpyrrolidin-2-yl)methanol (52 mg, 0.40 mmol) in THF (5 mL) was added sodium hydride (60%) (24 mg, 0.60 mmol) at 0° C. The mixture was stirred at room temperature for 1 h. Then tert-butyl-3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (67 mg, 0.10 mmol) in tetrahydrofuran (1 mL) was added dropwise and it was stirred at room temperature for another 1 h. The mixture was diluted with dichloromethane and water. The organic layer was combined, dried over sodium sulfate and evaporated. The residue was purified by preparative thin layer chromatography to give the title product (20 mg, 25%). MS (ESI, m/e) [M+1]⁺790.4.

### Step 2: 4-(4-(3,8-diazabicyclo[3.2.1]octan-3-yl)-6-chloro-8-fluoro-2-(((2S,4R)-4-fluoro-1-methylpyrrolidin-2-yl)methoxy)quinazolin-7-yl)-7-fluorobenzo[d]thiazol-2-amine

To a solution of tert-butyl-3-(7-(2-((tert-butoxycarbonyl)amino)-7-fluorobenzo[d]thiazol-4-yl)-6-chloro-8-fluoro-2-(((2S,4R)-4-fluoro-1-methylpyrrolidin-2-yl)methoxy)quinazolin-4-yl)-3,8-diazabicyclo[3.2.1]octane-8-carboxylate (20 mg, 0.025 mmol) in dichloromethane (2 mL) was added trifluoroacetic acid (1.5 mL) and it was stirred at room temperature for 3 hrs. Solvent was evaporated and the residue was adjusted to pH=11 with aq. Potassium carbonate, and it was diluted with dichloromethane:methanol=10:1 solvent and water. Combined organic layer was dried over sodium sulfate and evaporated. The residue was purified by preparative thin layer chromatography to give the title product (3 mg, 20%). ¹H NMR (500 MHz, CD₃OD) δ 7.88-7.85 (m, 1H), 7.24-7.18 (m, 1H), 7.01-6.94 (m, 1H), 5.26-5.10 (m, 1H), 4.59-4.44 (m, 4H), 3.84-3.57 (m, 4H), 3.60-3.44 (m, 1H), 3.22-3.11 (m, 1H), 2.75-2.60 (m, 1H), 2.38-2.15 (m, 1H), 2.40-2.30 (m, 2H), 2.07-1.83 (m, 6H). MS (ESI, m/e) [M+H]⁺ 590.4.

### Example 16: 4-(4-(3,8-diazabicyclo[3.2.1]octan-3-yl)-6-chloro-8-fluoro-2-(((S)-1-methyl pyrrolidin-2-yl)methoxy)quinazolin-7-yl)-7-fluorobenzo[d]thiazol-2-amine

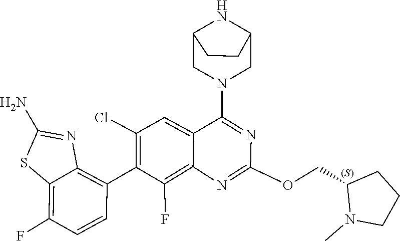

### Step 1: tert-butyl 3-(7-(2-((tert-butoxycarbonyl)amino)-7-fluorobenzo[d]thiazol-4-yl)-6-chloro-8-fluoro-2-(((S)-1-methylpyrrolidin-2-yl)methoxy)quinazolin-4-yl)-3,8-diazabicyclo[3.2.1]octane-8-carboxylate

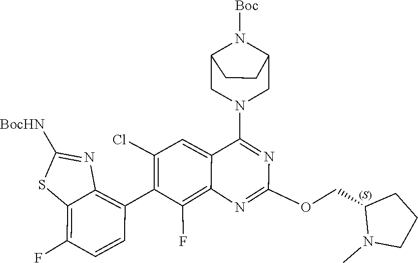

To a solution of (S)-(1-methylpyrrolidin-2-yl)methanol (51 mg, 0.444 mmol) in THF (7.5 mL) was added TBAI (5.5 mg, 0.0148 mmol) and NaH (24 mg, 0.592 mmol). The mixture was stirred at room temperature for about 20 mins. Then, tert-butyl 3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (100 mg, 0.148 mmol) in THF (7.5 mL) was added. The mixture was stirred at 40° C. for about 1.5 hrs and quenched by saturated NH₄Cl aqueous solution, extracted by ethyl acetate. The organic layers were washed by brine, dried over anhydrous Na₂SO₄ and evaporated in vacuo. The residue was purified by chromatography (PE/EtOAc=2:1 to 0:1, then DCM/MeOH=1:0 to 4:1) to give the crude product which was used directly in the next step.

### Step 2: 4-(4-(3,8-diazabicyclo[3.2.1]octan-3-yl)-6-chloro-8-fluoro-2-(((S)-1-methyl pyrrolidin-2-yl)methoxy)quinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-6-chloro-8-fluoro-2-(((S)-1-methylpyrrolidin-2-yl)methoxy)quinazolin-4-yl)-3,8-diazabicyclo[3.2.1]octane-8-carboxylate (100 mg, 0.130 mmol) in DCM (10 mL) was added TFA (2 mL). The mixture was stirred at room temperature for 21 hrs. Then, the mixture was evaporated in vacuo. The residue was re-dissolved in MeOH and 7 M NH₃ in MeOH was added to adjust pH >7. The solvent was removed in vacuo and the residue was purified by Prep-HPLC to give the product (26.76 mg, 36%). ¹H NMR (500 MHz, CD₃OD) δ 7.95 (s, 1H), 7.22 (dd, J=8.4, 5.4 Hz, 1H), 7.02 (t, J=8.6 Hz, 1H), 4.94-4.88 (m, 1H), 4.75-4.59 (m, 3H), 4.29-4.13 (m, 2H), 3.96-3.79 (m, 3H), 3.78-3.64 (m, 1H), 3.17 (m, 1H), 3.10 (s, 3H), 2.50-2.33 (m, 1H), 2.29-2.18 (m, 1H), 2.14 (s, 4H), 2.11-1.96 (m, 2H). MS (ESI, m/e) [M+H]⁺ 572.3.

### Example 17: 4-(4-(3,8-diazabicyclo[3.2.1]octan-3-yl)-6-chloro-2-(((S)-1-(4,4-difluoropiperidin-1-yl)propan-2-yl)oxy)-8-fluoroquinazolin-7-yl)-7-fluorobenzo[d]thiazol-2-amine

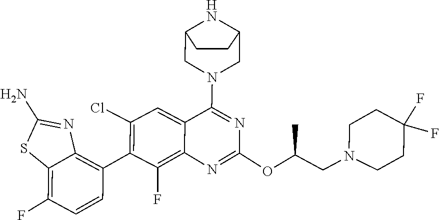 [SMILES: C[C@@H](CN1CCC(F)(F)CC1)Oc1nc(N2CC3CCC(C2)N3)c2cc(Cl)c(-c3ccc(F)c4sc(N)nc34)c(F)c2n1]

### Step 1: (5)-1-(4,4-difluoropiperidin-1-yl)propan-2-ol

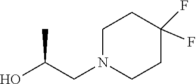

To a solution of 4,4-difluoropiperidine (600 mg, 5.0 mmol) in water (5 mL) was added (S)-2-methyloxirane (290 mg, 5 mmol) dropwise at 0° C. The mixture was stirred at room temperature for 16 h. The reaction was extracted with ethyl ester (15 mL), the organic layer was combined, dried over sodium sulfate and evaporated to give crude product which was used directly in the next step. MS (ESI, m/e) [M+1]⁺180.1.

### Step 2: tert-butyl 3-(7-(2-((tert-butoxycarbonyl)amino)-7-fluorobenzo[d]thiazol-4-yl)-6-chloro-2-(((S)-1-(4,4-difluoropiperidin-1-yl)propan-2-yl)oxy)-8-fluoroquinazolin-4-yl)-3,8-diazabicyclo[3.2.1]octane-8-carboxylate

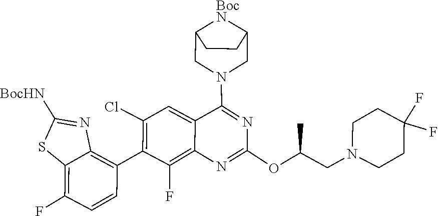 [SMILES: C[C@@H](CN1CCC(F)(F)CC1)Oc1nc(N2CC3CCC(C2)N3C(=O)OC(C)(C)C)c2cc(Cl)c(-c3ccc(F)c4sc(NC(=O)OC(C)(C)C)nc34)c(F)c2n1]

To a solution of (S)-1-(4,4-difluoropiperidin-1-yl)propan-2-ol (200 mg, 1.11 mmol) in 3 mL tetrahydrofuran was added sodium hydride (60%) (60 mg, 1.50 mmol) at 0° C. The mixture was stirred at room temperature for 1 h. Then tert-butyl-3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (130 mg, 0.19 mmol) in THF (1 mL) was added dropwise and it was stirred at room temperature for another 1 h. The mixture was diluted with dichloromethane and water. The organic layer was combined, dried over sodium sulfate and evaporated. The residue was purified by preparative thin layer chromatography to give the title product (40 mg, 25%). MS (ESI, m/e) [M+1]⁺836.4.

### Step 3: 4-(4-(3,8-diazabicyclo[3.2.1]octan-3-yl)-6-chloro-2-(((S)-1-(4,4-difluoropiperidin-1-yl)propan-2-yl)oxy)-8-fluoroquinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-6-chloro-2-(((S)-1-(4,4-difluoropiperidin-1-yl)propan-2-yl)oxy)-8-fluoroquinazolin-4-yl)-3,8-diazabicyclo[3.2.1]octane-8-carboxylate (40 mg, 0.048 mmol) in dichloromethane (3 mL) was added trifluoroacetic acid (1.5 mL) and the mixture was stirred at room temperature for 3 h. Solvent was evaporated and the residue was adjusted to pH=11 with aq. Potassium carbonate and it was diluted with dichloromethane:methanol=10:1 solvent and water. Combined organic layer was dried over sodium sulfate and evaporated. The residue was lyophilized to give the title product (10 mg, 33%). ¹H NMR (500 MHz, CD₃OD) δ 7.86 (s, 1H), 7.23-7.16 (m, 1H), 7.02-6.94 (m, 1H), 5.55-5.46 (m, 1H), 4.55-4.35 (m, 2H), 3.69-3.54 (m, 4H), 2.86-2.76 (m, 1H), 2.76-2.63 (m, 4H), 2.61-2.54 (m, 1H), 1.96-1.75 (m, 8H), 1.38-1.37 (d, J=1.4 Hz, 3H). MS (ESI, m/e) [M+H]⁺ 636.3.

### Example 18: 4-(4-(3,8-diazabicyclo[3.2.1]octan-8-yl)-6-chloro-8-fluoro-2-(((S)-1-(piperidin-1-yl)propan-2-yl)oxy)quinazolin-7-yl)-7-fluorobenzo[d]thiazol-2-amine

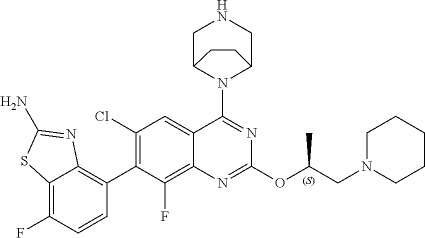

### Step 1: tert-butyl 8-(7-(2-((tert-butoxycarbonyl)amino)-7-fluorobenzo[d]thiazol-4-yl)-6-chloro-8-fluoro-2-(((S)-1-(piperidin-1-yl)propan-2-yl)oxy)quinazolin-4-yl)-3,8-diazabicyclo [3.2.1]octane-3-carboxylate

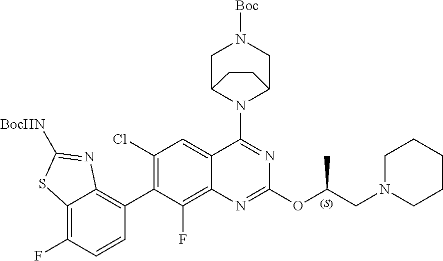

To a solution of known compound (S)-1-(piperidin-1-yl)propan-2-ol (127 mg, 0.888 mmol) in THF (15 mL) was added TBAI (11 mg, 0.03 mmol) and NaH (47 mg, 1.184 mmol). The mixture was stirred at room temperature for about 40 mins. Then, tert-butyl 8-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-3-carboxylate (200 mg, 0.296 mmol) in THF (15 mL) was added to the mixture. The mixture was stirred at 40° C. for about 2.5 hrs and quenched by saturated NH₄Cl aqueous solution, extracted by ethyl acetate. The organic layers were washed by brine, dried over anhydrous Na₂SO₄, evaporated in vacuo. The residue was purified by chromatography (PE/EtOAc=2:1 to 0:1, then DCM/MeOH=1:0 to 4:1) to give the product which was used directly in the next step.

### Step 2: 4-(4-(3,8-diazabicyclo[3.2.1]octan-8-yl)-6-chloro-8-fluoro-2-(((S)-1-(piperidin-1-yl)propan-2-yl)oxy)quinazolin-7-yl)-7-fluorobenzo[d]thiazol-2-amine

To a solution of tert-butyl 8-(7-(2-((tert-butoxycarbonyl)amino)-7-fluorobenzo[d]thiazol-4-yl)-6-chloro-8-fluoro-2-(((S)-1-(piperidin-1-yl)propan-2-yl)oxy)quinazolin-4-yl)-3,8-diazabicyclo[3.2.1]octane-3-carboxylate (255 mg, 0.136 mmol) in DCM (20 mL) was added TFA (4 mL). The mixture was stirred at room temperature for overnight. Then, the mixture was evaporated in vacuo. The residue was re-dissolved in MeOH and 7 M NH₃ in MeOH was added to adjust pH >7. Then, the solvent was removed in vacuo and the residue was purified by Prep-HPLC to give the product (39.32 mg, 19%). ¹H NMR (500 MHz, CD₃OD) δ 8.40 (s, 1H), 7.98 (s, 1H), 7.24 (dd, J=8.2, 5.3 Hz, 1H), 7.01 (t, J=8.8 Hz, 1H), 5.72-5.49 (m, 1H), 5.05 (d, J=15.4 Hz, 2H), 3.58-3.42 (m, 4H), 3.36 (d, J=13.8 Hz, 2H), 3.22 (d, J=12.6 Hz, 4H), 2.22-2.17 (m, 2H), 2.13 (d, J=8.3 Hz, 2H), 1.83 (d, J=4.8 Hz, 4H), 1.70-1.55 (m, 2H), 1.48 (d, J=6.2 Hz, 3H). MS (ESI, m/e) [M+H]⁺ 600.3.

### Example 19: 4-(4-(3,8-diazabicyclo[3.2.1]octan-8-yl)-6-chloro-2-(((S)-1-(4,4-difluoropiperidin-1-yl)propan-2-yl)oxy)-8-fluoroquinazolin-7-yl)-7-fluorobenzo[d]thiazol-2-amine

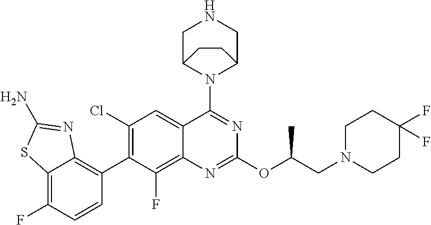 [SMILES: C[C@@H](CN1CCC(F)(F)CC1)Oc1nc(N2C3CCC2CNC3)c2cc(Cl)c(-c3ccc(F)c4sc(N)nc34)c(F)c2n1]

### Step 1: tert-butyl 8-(7-(2-((tert-butoxycarbonyl)amino)-7-fluorobenzo[d]thiazol-4-yl)-6-chloro-2-(((S)-1-(4,4-difluoropiperidin-1-yl)propan-2-yl)oxy)-8-fluoroquinazolin-4-yl)-3,8-diazabicyclo[3.2.1]octane-3-carboxylate

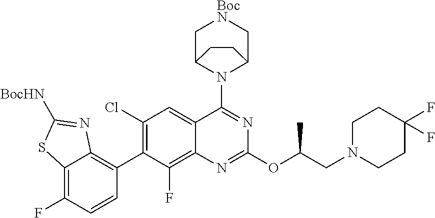 [SMILES: C[C@@H](CN1CCC(F)(F)CC1)Oc1nc(N2C3CCC2CN(C(=O)OC(C)(C)C)C3)c2cc(Cl)c(-c3ccc(F)c4sc(NC(=O)OC(C)(C)C)nc34)c(F)c2n1]

To a solution of (S)-1-(4,4-difluoropiperidin-1-yl)propan-2-ol (160 mg, 0.89 mmol) in 3 mL tetrahydrofuran was added sodium hydride (60%) (48 mg, 1.20 mmol) at 0° C. The mixture was stirred at room temperature for 1 h. Then tert-butyl (8-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-3-carboxylate (110 mg, 0.16 mmol) in 1 mL tetrahydrofuran was added dropwise and it was stirred at room temperature for another 1 h. And it was diluted with dichloromethane and water. The organic layer was combined, dried over sodium sulfate and evaporated. The residue was purified by preparative thin layer chromatography to give the title product (36 mg, 26%). MS (ESI, m/e) [M+1]⁺836.4.

### Step 2: 4-(4-(3,8-diazabicyclo[3.2.1]octan-8-yl)-6-chloro-2-(((S)-1-(4,4-difluoropiperidin-1-yl)propan-2-yl)oxy)-8-fluoroquinazolin-7-yl)-7-fluorobenzo[d]thiazol-2-amine

To a solution of tert-butyl 8-(7-(2-((tert-butoxycarbonyl)amino)-7-fluorobenzo[d]thiazol-4-yl)-6-chloro-2-(((S)-1-(4,4-difluoropiperidin-1-yl)propan-2-yl)oxy)-8-fluoroquinazolin-4-yl)-3,8-diazabicyclo[3.2.1]octane-3-carboxylate (30 mg, 0.036 mmol) in dichloromethane (3 mL) was added trifluoroacetic acid (1.5 mL) and it was stirred at room temperature for 4 hrs. Solvent was evaporated and the residue was adjusted pH=11 with aq. Potassium carbonate and it was diluted with dichloromethane:methanol=10:1 solvent and water. Combined organic layer was dried over sodium sulfate and evaporated. The residue was lyophilized to give the title product (22 mg, 88%). ¹H NMR (500 MHz, CD₃OD) δ 7.89 (s, 1H), 7.23-7.16 (m, 1H), 7.02-6.94 (m, 1H), 5.55-5.46 (m, 1H), 4.92-4.86 (m, 1H), 3.27-3.20 (m, 2H), 3.13-3.03 (m, 1H), 2.93-2.87 (m, 2H), 2.74-2.61 (m, 4H), 2.60-2.54 (m, 1H), 2.12-1.75 (m, 9H), 1.38-1.37 (d, J=1.4 Hz, 3H). MS (ESI, m/e) [M+H]⁺ 636.3.

### Example 20: 4-(4-(3,8-diazabicyclo[3.2.1]octan-3-yl)-6-chloro-2-(((S)-1-(3,3-difluoropyrrolidin-1-yl)propan-2-yl)oxy)-8-fluoroquinazolin-7-yl)-7-fluorobenzo[d]thiazol-2-amine

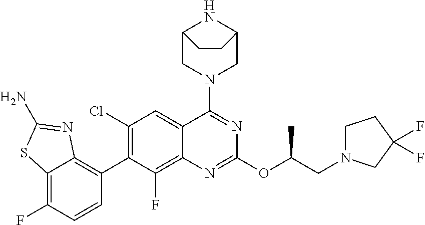 [SMILES: C[C@@H](CN1CCC(F)(F)C1)Oc1nc(N2CC3CCC(C2)N3)c2cc(Cl)c(-c3ccc(F)c4sc(N)nc34)c(F)c2n1]

### Step 1 (S)-1-(3,3-difluoropyrrolidin-1-yl)propan-2-ol

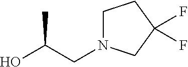

To a solution of 3,3-difluoropyrrolidine hydrogen chloride (600 mg, 4.20 mmol) in water (5 mL) was adjusted pH to 11 with potassium carbonate and it was added (S)-2-methyloxirane (300 mg, 5.00 mmol) at 0° C. The mixture was stirred at room temperature for 16 h. Then it was diluted with ethyl ether. The organic layer was combined, dried over sodium sulfate and evaporated to give crude product which was used directly in the next step. MS (ESI, m/e) [M+1]⁺166.1.

### Step 2: tert-butyl 3-(7-(2-((tert-butoxycarbonyl)amino)-7-fluorobenzo[d]thiazol-4-yl)-6-chloro-2-(((S)-1-(3,3-difluoropyrrolidin-1-yl)propan-2-yl)oxy)-8-fluoroquinazolin-4-yl)-3,8-diazabicyclo[3.2.1]octane-8-carboxylate

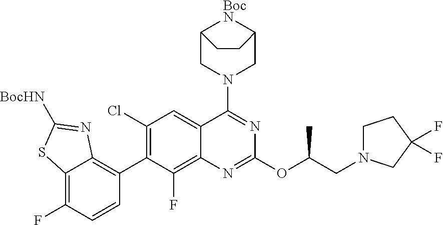 [SMILES: C[C@@H](CN1CCC(F)(F)C1)Oc1nc(N2CC3CCC(C2)N3C(=O)OC(C)(C)C)c2cc(Cl)c(-c3ccc(F)c4sc(NC(=O)OC(C)(C)C)nc34)c(F)c2n1]

To a solution of (S)-1-(3,3-difluoropyrrolidin-1-yl)propan-2-ol (140 mg, 0.88 mmol) in THF (5 mL) was added sodium hydride (60%) (48 mg, 1.20 mmol) at 0° C. The mixture was stirred at room temperature for 1 h. Then tert-butyl-3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (110 mg, 0.16 mmol) in 1 mL THF was added dropwise and it was stirred at room temperature for another 1 h. And it was diluted with dichloromethane and water. The organic layer was combined, dried over sodium sulfate and evaporated. The residue was purified by preparative thin layer chromatography to give the title product (30 mg, 22%). MS (ESI, m/e) [M+1]⁺822.4.

### Step 3: 4-(4-(3,8-diazabicyclo[3.2.1]octan-3-yl)-6-chloro-2-(((S)-1-(3,3-difluoropyrrolidin-1-yl)propan-2-yl)oxy)-8-fluoroquinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-6-chloro-2-(((S)-1-(3,3-difluoropyrrolidin-1-yl)propan-2-yl)oxy)-8-fluoroquinazolin-4-yl)-3,8-diazabicyclo[3.2.1]octane-8-carboxylate (30 mg, 0.036 mmol) in dichloromethane (3 mL) was added trifluoroacetic acid (1.5 mL) and it was stirred at room temperature for 4 h. Solvent was evaporated and the residue was adjusted to pH=11 with aq. Potassium carbonate and it was diluted with dichloromethane:methanol=10:1 solvent and water. Combined organic layer was dried over sodium sulfate and evaporated. The residue was lyophilized to give the title product (20 mg, 88%). ¹H NMR (400 MHz, CD₃OD) δ 7.86 (s, 1H), 7.23-7.16 (m, 1H), 7.02-6.94 (m, 1H), 5.48-5.43 (m, 1H), 4.56-4.35 (m, 2H), 3.69-3.54 (m, 4H), 3.08-2.93 (m, 2H), 2.91-2.78 (m, 3H), 2.77-2.69 (m, 1H), 2.25-2.10 (m, 2H), 1.90-1.72 (m, 4H), 1.39-1.38 (d, J=1.4 Hz, 3H). MS (ESI, m/e) [M+H]⁺ 622.3.

### Example 21: 4-(4-(3-oxa-7,9-diazabicyclo[3.3.1]nonan-7-yl)-6-chloro-8-fluoro-2-((1-(piperidin-1-yl)propan-2-yl)oxy)quinazolin-7-yl)-7-fluorobenzo[d]thiazol-2-amine

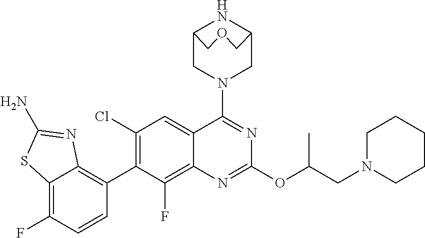 [SMILES: CC(CN1CCCCC1)Oc1nc(N2CC3COCC(C2)N3)c2cc(Cl)c(-c3ccc(F)c4sc(N)nc34)c(F)c2n1]

### Step 1: tert-butyl 7-(7-bromo-2,6-dichloro-8-fluoroquinazolin-4-yl)-3-oxa-7,9-diazabicyclo[3.3.1]nonane-9-carboxylate

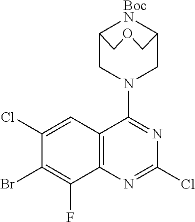 [SMILES: CC(C)(C)OC(=O)N1C2COCC1CN(c1nc(Cl)nc3c(F)c(Br)c(Cl)cc13)C2]

To a solution of 7-bromo-2,4,6-trichloro-8-fluoroquinazoline (500 mg, 1.51 mmol) and DIPEA (0.65 mL, 3.77 mmol) in DCM (50 mL) was added tert-butyl 3-oxa-7,9-diazabicyclo[3.3.1]nonane-9-carboxylate (380 mg, 1.67 mmol), and the mixture was stirred at room temperature for 3 hrs. Upon completion, solvent was removed and crude product was purified by chromatography column on silica (eluting with DCM/MeOH=40/1) to give the title product (600 mg, 76% yield). MS (ESI, m/e) [M+1]⁺521.4.

### Step 2: tert-butyl 7-(7-bromo-6-chloro-2,8-difluoroquinazolin-4-yl)-3-oxa-7,9-diazabicyclo[3.3.1]nonane-9-carboxylate

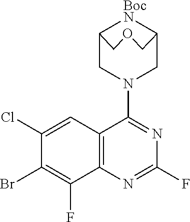 [SMILES: CC(C)(C)OC(=O)N1C2COCC1CN(c1nc(F)nc3c(F)c(Br)c(Cl)cc13)C2]

A solution of tert-butyl 7-(7-bromo-2,6-dichloro-8-fluoroquinazolin-4-yl)-3-oxa-7,9-diazabicyclo[3.3.1]nonane-9-carboxylate (300 mg, 0.574 mmol) and KF (333 mg, 5.74 mmol) in DMSO (50 mL) was stirred at 95° C. overnight. Upon completion, solvent was removed and crude product was purified by chromatography column on silica (eluting with DCM/MeOH=20/1) to give the title product (180 mg, 62% yield). MS (ESI, m/e) [M+1]⁺507.4.

### Step 3: tert-butyl 7-(7-bromo-6-chloro-8-fluoro-2-((1-(piperidin-1-yl)propan-2-yl)oxy)quinazolin-4-yl)-3-oxa-7,9-diazabicyclo[3.3.1]nonane-9-carboxylate

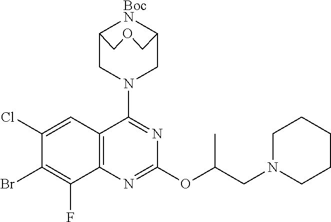 [SMILES: CC(CN1CCCCC1)Oc1nc(N2CC3COCC(C2)N3C(=O)OC(C)(C)C)c2cc(Cl)c(Br)c(F)c2n1]

To a solution of 1-(piperidin-1-yl)propan-2-ol (113 mg, 0.79 mmol) in THF (20 mL) was added NaH (32 mg, 0.79 mmol) in THF (50 mL), and the mixture was stirred at room temperature for 1 h. Then tert-butyl 7-(7-bromo-6-chloro-2,8-difluoroquinazolin-4-yl)-3-oxa-7,9-diazabicyclo[3.3.1]nonane-9-carboxylate (200 mg, 0.39 mmol) was added, and the mixture was stirred at room temperature for 5 hrs. Upon completion, solvent was removed and crude product was purified by chromatography column on silica (eluting with DCM/MeOH=20/1) to give the title product (156 mg, 63% yield). MS (ESI, m/e) [M+1]⁺628.4.

### Step 4: tert-butyl 7-(7-(2-((tert-butoxycarbonyl)amino)-7-fluorobenzo[d]thiazol-4-yl)-6-chloro-8-fluoro-2-((1-(piperidin-1-yl)propan-2-yl)oxy)quinazolin-4-yl)-3-oxa-7,9-diazabicyclo[33.3.1]nonane-9-carboxylate

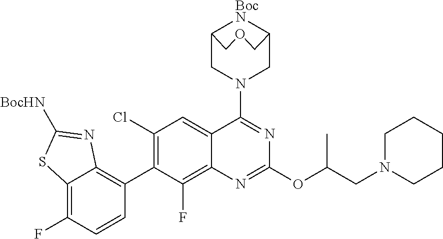 [SMILES: CC(CN1CCCCC1)Oc1nc(N2CC3COCC(C2)N3C(=O)OC(C)(C)C)c2cc(Cl)c(-c3ccc(F)c4sc(NC(=O)OC(C)(C)C)nc34)c(F)c2n1]

A solution of tert-butyl 7-(7-bromo-6-chloro-8-fluoro-2-((1-(piperidin-1-yl)propan-2-yl)oxy)quinazolin-4-yl)-3-oxa-7,9-diazabicyclo[3.3.1]nonane-9-carboxylate (85 mg, 0.14 mmol), (2-((tert-butoxycarbonyl)amino)-7-fluorobenzo[d]thiazol-4-yl)boronic acid (65 mg, 0.21 mmol), Pd(dppf)₂Cl₂ (12 mg, 0.014 mmol) and NaHCO₃ (30 mg, 0.35 mmol) in dioxane/H₂O (10 mL/1 mL) was stirred at 85° C. overnight. Upon completion, solvent was removed and crude product was purified by chromatography column on silica (eluting with DCM/MeOH=20/1) to give the title product (60 mg, 53% yield). MS (ESI, m/e) [M+1]⁺816.4.

### Step 5: 4-(4-(3-oxa-7,9-diazabicyclo[3.3.1]nonan-7-yl)-6-chloro-8-fluoro-2-((1-(piperidin-1-yl)propan-2-yl)oxy)quinazolin-7-yl)-7-fluorobenzo[d]thiazol-2-amine

To a solution of tert-butyl 7-(7-(2-((tert-butoxycarbonyl)amino)-7-fluorobenzo[d]thiazol-4-yl)-6-chloro-8-fluoro-2-((1-(piperidin-1-yl)propan-2-yl)oxy)quinazolin-4-yl)-3-oxa-7,9-diazabicyclo[3.3.1]nonane-9-carboxylate (60 mg, 0.074 mmol) in DCM (5 mL) was added TFA (5 mL), and the mixture was stirred at room temperature for 2 h. Upon completion, solvent was removed and crude product was purified by prep-HPLC to give the title product (15 mg, 33% yield). ¹H NMR (400 MHz, CD₃OD) δ 7.99 (d, J=3.9 Hz, 1H), 7.22 (td, J=8.3, 5.5 Hz, 1H), 7.00 (td, J=8.8, 2.3 Hz, 1H), 5.68-5.61 (m, 1H), 5.04-4.93 (m, 2H), 4.09-3.97 (m, 6H), 3.55-3.50 (m, 2H), 3.44-3.39 (m, 2H), 3.35-3.30 (m, 4H), 1.93-1.84 (m, 6H), 1.48 (d, J=6.3 Hz, 3H). MS (ESI, m/e) [M+1]⁺616.4.

### Example 22: 4-((2S)-2-((7-(2-amino-7-fluorobenzo[d]thiazol-4-yl)-4-(3,8-diazabicyclo[3.2.1]octan-3-yl)-6-chloro-8-fluoroquinazolin-2-yl)oxy)propyl)thiomorpholine 1,1-dioxide

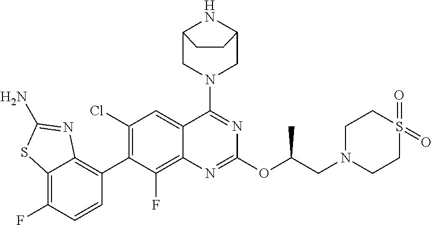 [SMILES: C[C@@H](CN1CCS(=O)(=O)CC1)Oc1nc(N2CC3CCC(C2)N3)c2cc(Cl)c(-c3ccc(F)c4sc(N)nc34)c(F)c2n1]

### Step 1: (S)-4-(2-hydroxypropyl)thiomorpholine 1,1-dioxide

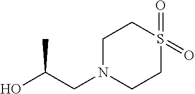

To a solution of thiomorpholine 1,1-dioxide (700 mg, 5.22 mmol) in water (5 mL) was added (S)-2-methyloxirane (290 mg, 5 mmol) dropwise at 0° C. The mixture was stirred at room temperature for 16 hrs. The reaction was diluted with dichloromethane:methanol=10:1 solvent. Combined organic layer was dried over sodium sulfate and evaporated to give crude product which was used directly in the next step. MS (ESI, m/e) [M+1]⁺193.9.

### Step 2: tert-butyl 3-(7-(2-((tert-butoxycarbonyl)amino)-7-fluorobenzo[d]thiazol-4-yl)-6-chloro-2-(((S)-1-(1,1-dioxidothiomorpholino)propan-2-yl)oxy)-8-fluoroquinazolin-4-yl)-3,8-diazabicyclo[3.2.1]octane-8-carboxylate

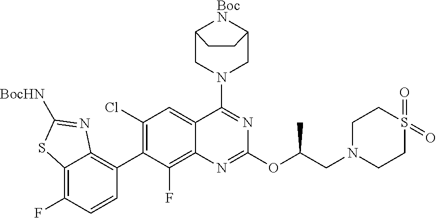 [SMILES: C[C@@H](CN1CCS(=O)(=O)CC1)Oc1nc(N2CC3CCC(C2)N3C(=O)OC(C)(C)C)c2cc(Cl)c(-c3ccc(F)c4sc(NC(=O)OC(C)(C)C)nc34)c(F)c2n1]

To a solution of (S)-4-(2-hydroxypropyl)thiomorpholine 1,1-dioxide (150 mg, 0.77 mmol) in 5 mL tetrahydrofuran was added sodium hydride (60%) (40 mg, 1.00 mmol) at 0° C. The mixture was stirred at room temperature for 1 h. Then tert-butyl-3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (100 mg, 0.15 mmol) in 1 mL tetrahydrofuran was added dropwise and it was stirred at room temperature for another 1 h. The mixture was diluted with dichloromethane and water. Combined organic layer was dried over sodium sulfate and evaporated. The residue was purified by preparative thin layer chromatography to give the title product (60 mg, 48%). MS (ESI, m/e) [M+1]⁺850.4.

### Step 3: 4-((2S)-2-((7-(2-amino-7-fluorobenzo[d]thiazol-4-yl)-4-(3,8-diazabicyclo[3.2.1]octan-3-yl)-6-chloro-8-fluoroquinazolin-2-yl)oxy)propyl)thiomorpholine 1,1-dioxide

To a solution of tert-butyl 3-(7-(2-((tert-butoxycarbonyl)amino)-7-fluorobenzo[d]thiazol-4-yl)-6-chloro-2-(((S)-1-(1,1-dioxidothiomorpholino)propan-2-yl)oxy)-8-fluoroquinazolin-4-yl)-3,8-diazabicyclo[3.2.1]octane-8-carboxylate (40 mg, 0.048 mmol) in dichloromethane (1.5 mL) was added trifluoroacetic acid (1.0 mL) and it was stirred at room temperature for 3 hrs. Solvent was evaporated and the residue was adjusted to pH=11 with aq. Potassium carbonate and it was diluted with dichloromethane:methanol=10:1 solvent and water. Combined organic layer was dried over sodium sulfate and evaporated. The residue was lyophilized to give the title product (25 mg, 82%). ¹H NMR (500 MHz, CD₃OD) δ 7.86 (s, 1H), 7.25-7.18 (m, 1H), 7.01-6.94 (m, 1H), 5.58-5.46 (m, 1H), 4.48-4.37 (m, 2H), 3.67-3.54 (m, 4H), 3.27-3.14 (m, 2H), 3.12-2.94 (m, 6H), 2.93-2.84 (m, 1H), 2.75-2.63 (m, 1H), 1.90-1.76 (m, 4H), 1.39-1.38 (d, J=1.4 Hz, 3H). MS (ESI, m/e) [M+H]⁺ 650.4.

### Example 23: 4-(2-(((S)-1-(azetidin-1-yl)propan-2-yl)oxy)-4-(3,8-diazabicyclo[3.2.1]octan-3-yl)-6-chloro-8-fluoroquinazolin-7-yl)-7-fluorobenzo[d]thiazol-2-amine

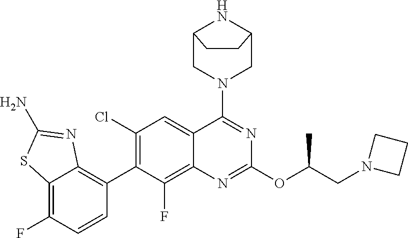 [SMILES: C[C@@H](CN1CCC1)Oc1nc(N2CC3CCC(C2)N3)c2cc(Cl)c(-c3ccc(F)c4sc(N)nc34)c(F)c2n1]

### Step 1: (S)-1-(azetidin-1-yl)propan-2-ol

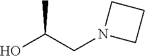

To a solution of azetidine (290 mg, 5.00 mmol) in 5 mL water was added (S)-2-methyloxirane (290 mg, 5 mmol) dropwise at 0° C. The mixture was stirred at room temperature for 16 h. The reaction was diluted with dichloromethane:methanol=10:1 solvent, the organic layer was combined, dried over sodium sulfate and evaporated to give crude product which was used directly in the next step. MS (ESI, m/e) [M+1]⁺116.2.

### Step 2: tert-butyl 3-(2-(((S)-1-(azetidin-1-yl)propan-2-yl)oxy)-7-(2-((tert-butoxycarbonyl)amino)-7-fluorobenzo[d]thiazol-4-yl)-6-chloro-8-fluoroquinazolin-4-yl)-3,8-diazabicyclo[3.2.1]octane-8-carboxylate

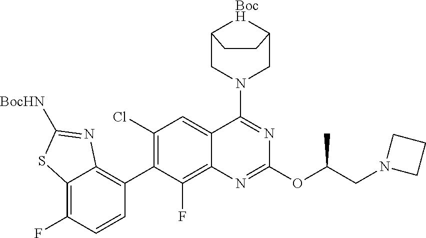

To a solution of (S)-1-(azetidin-1-yl)propan-2-ol (30 mg, 0.26 mmol) in 2 mL tetrahydrofuran was added sodium hydride (60%) (12 mg, 0.30 mmol) at 0° C. The mixture was stirred at room temperature for 1 h. Then tert-butyl 3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (55 mg, 0.081 mmol) in 1 mL tetrahydrofuran was added dropwise and the mixture was stirred at room temperature for another 1 h. The mixture was diluted with dichloromethane and water. Combined organic layer was dried over sodium sulfate and evaporated. The residue was purified by preparative thin layer chromatography to give the title product (16 mg, 25%). MS (ESI, m/e) [M+1]⁺772.4.

### Step 3: 4-(2-(((S)-1-(azetidin-1-yl)propan-2-yl)oxy)-4-(3,8-diazabicyclo[3.2.1]octan-3-yl)-6-chloro-8-fluoroquinazolin-7-yl)-7-fluorobenzo[d]thiazol-2-amine

To a solution of tert-butyl 3-(2-(((S)-1-(azetidin-1-yl)propan-2-yl)oxy)-7-(2-((tert-butoxycarbonyl)amino)-7-fluorobenzo[d]thiazol-4-yl)-6-chloro-8-fluoroquinazolin-4-yl)-3,8-diazabicyclo[3.2.1]octane-8-carboxylate (16 mg, 0.02 mmol) in dichloromethane (1.5 mL) was added trifluoroacetic acid (1.0 mL) and it was stirred at room temperature for 3 hrs. Solvent was evaporated and the residue was adjusted to pH=11 with aq. Potassium carbonate and it was diluted with dichloromethane:methanol=10:1 solvent and water. Combined organic layer was dried over sodium sulfate and evaporated. The residue was lyophilized to give the title product (10 mg, 84%) as white solid. ¹H NMR (500 MHz, CD₃OD) δ 7.86 (s, 1H), 7.23-7.17 (m, 1H), 7.01-6.96 (m, 1H), 5.35-5.29 (m, 1H), 4.59-4.39 (m, 2H), 3.70-3.50 (m, 4H), 3.49-3.35 (m, 4H), 2.95-2.70 (m, 2H), 2.16-2.02 (m, 2H), 1.90-1.67 (m, 4H) 1.34-1.33 (d, J=1.4 Hz, 3H). MS (ESI, m/e) [M+H]⁺ 572.4.

### Example 24: 4-(4-(3,8-diazabicyclo[3.2.1]octan-3-yl)-6-chloro-8-fluoro-2-(((2S)-1-(3-fluoropyrrolidin-1-yl)propan-2-yl)oxy)quinazolin-7-yl)-7-fluorobenzo[d]thiazol-2-amine

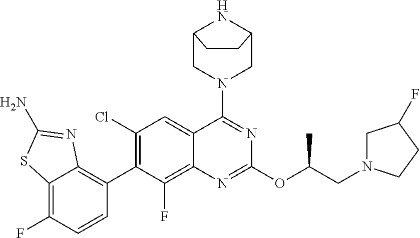 [SMILES: C[C@@H](CN1CCC(F)C1)Oc1nc(N2CC3CCC(C2)N3)c2cc(Cl)c(-c3ccc(F)c4sc(N)nc34)c(F)c2n1]

### Step 1: (2S)-1-(3-fluoropyrrolidin-1-yl)propan-2-ol

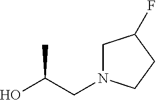

A solution of 3-fluoropyrrolidine hydrogen chloride (600 mg, 4.80 mmol) in water (5 mL) was adjusted pH to 11 with potassium carbonate. (S)-2-methyloxirane (300 mg, 5.00 mmol) was then added at 0° C. and the mixture was stirred at room temperature for 16 hrs. The mixture was diluted with dichloromethane. Combined organic layer was dried over sodium sulfate and evaporated to give crude product which was used directly in the next step. MS (ESI, m/e) [M+1]⁺148.2.

### Step 2: tert-butyl 3-(7-(2-((tert-butoxycarbonyl)amino)-7-fluorobenzo[d]thiazol-4-yl)-6-chloro-8-fluoro-2-(((2S)-1-(3-fluoropyrrolidin-1-yl)propan-2-yl)oxy)quinazolin-4-yl)-3,8-diazabicyclo[3.2.1]octane-8-carboxylate

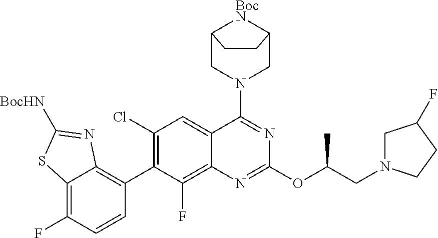 [SMILES: C[C@@H](CN1CCC(F)C1)Oc1nc(N2CC3CCC(C2)N3C(=O)OC(C)(C)C)c2cc(Cl)c(-c3ccc(F)c4sc(NC(=O)OC(C)(C)C)nc34)c(F)c2n1]

To a solution of (2S)-1-(3-fluoropyrrolidin-1-yl)propan-2-ol (110 mg, 0.75 mmol) in THF (5 mL) was added sodium hydride (60%) (40 mg, 1.00 mmol) at 0° C. The mixture was stirred at room temperature for 1 h. Then tert-butyl-3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (110 mg, 0.16 mmol) in 1 mL tetrahydrofuran was added dropwise and it was stirred at room temperature for another 1 h. The mixture was diluted with dichloromethane and water. Combined organic layer was dried over sodium sulfate and evaporated. The residue was purified by preparative thin layer chromatography to give the title product (30 mg, 23%). MS (ESI, m/e) [M+1]⁺804.4.

### Step 3: 4-(4-(3,8-diazabicyclo[3.2.1]octan-3-yl)-6-chloro-8-fluoro-2-(((2S)-1-(3-fluoropyrrolidin-1-yl)propan-2-yl)oxy)quinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-6-chloro-8-fluoro-2-(((2S)-1-(3-fluoropyrrolidin-1-yl)propan-2-yl)oxy)quinazolin-4-yl)-3,8-diazabicyclo[3.2.1]octane-8-carboxylate (30 mg, 0.037 mmol) in dichloromethane (1.5 mL) was added trifluoroacetic acid (1.0 mL) and it was stirred at room temperature for 3 hrs. Solvent was evaporated and the residue was adjusted to pH=11 with aq. Potassium carbonate and it was diluted with dichloromethane:methanol=10:1 solvent and water. Combined organic layer was dried over sodium sulfate and evaporated. The residue was lyophilized to give the title product (10 mg, 44%). ¹H NMR (400 MHz, CD₃OD) δ 7.86 (s, 1H), 7.23-7.16 (m, 1H), 7.02-6.94 (m, 1H), 5.52-5.43 (m, 1H), 5.22-5.00 (m, 1H), 4.57-4.35 (m, 2H), 3.72-3.48 (m, 4H), 3.10-2.72 (m, 5H), 2.65-2.44 (m, 1H), 2.25-2.06 (m, 1H), 2.01-1.72 (m, 5H), 1.39-1.38 (d, J=1.4 Hz, 3H). MS (ESI, m/e) [M+H]⁺ 604.3.

### Example 25: 4-(4-(3,8-diazabicyclo[3.2.1]octan-3-yl)-6-chloro-8-fluoro-2-(((S)-1-(4-fluoropiperidin-1-yl)propan-2-yl)oxy)quinazolin-7-yl-7-fluorobenzo[d]thiazol-2-amine

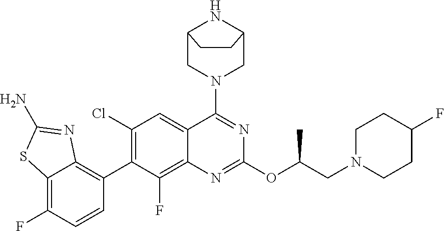 [SMILES: C[C@@H](CN1CCC(F)CC1)Oc1nc(N2CC3CCC(C2)N3)c2cc(Cl)c(-c3ccc(F)c4sc(N)nc34)c(F)c2n1]

### Step 1: (S)-1-(4-fluoropiperidin-1-yl)propan-2-ol

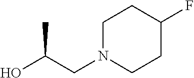

A solution of 4-fluoropiperidine hydrogen chloride (700 mg, 5.01 mmol) in 5 mL water was adjusted pH to 11 with potassium carbonate. (S)-2-methyloxirane (300 mg, 5.00 mmol) was then added at 0° C. The mixture was stirred at room temperature for 16 hrs. Then it was diluted with dichloromethane. Combined organic layer was dried over sodium sulfate and evaporated to give crude product which was used directly. MS (ESI, m/e) [M+1]⁺162.2.

### Step 2: tert-butyl 3-(7-(2-((tert-butoxycarbonyl)amino)-7-fluorobenzo[d]thiazol-4-yl)-6-chloro-8-fluoro-2-(((S)-1-(4-fluoropiperidin-1-yl)propan-2-yl)oxy)quinazolin-4-yl)-3,8-diazabicyclo[3.2.1]octane-8-carboxylate

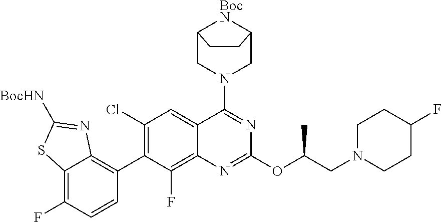 [SMILES: C[C@@H](CN1CCC(F)CC1)Oc1nc(N2CC3CCC(C2)N3C(=O)OC(C)(C)C)c2cc(Cl)c(-c3ccc(F)c4sc(NC(=O)OC(C)(C)C)nc34)c(F)c2n1]

To a solution of (S)-1-(4-fluoropiperidin-1-yl)propan-2-ol (110 mg, 0.68 mmol) in 5 mL tetrahydrofuran was added sodium hydride (60%) (40 mg, 1.00 mmol) at 0° C. The mixture was stirred at room temperature for 1 h. Then tert-butyl-3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (110 mg, 0.16 mmol) in 1 mL tetrahydrofuran was added dropwise and it was stirred at room temperature for another 1 h. The mixture was diluted with dichloromethane and water. Combined organic layer was dried over sodium sulfate and evaporated. The residue was purified by preparative thin layer chromatography to give the title product (30 mg, 22%). MS (ESI, m/e) [M+1]⁺818.4.

### Step 3: 4-(4-(3,8-diazabicyclo[3.2.1]octan-3-yl)-6-chloro-8-fluoro-2-(((S)-1-(4-fluoropiperidin-1-yl)propan-2-yl)oxy)quinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-6-chloro-8-fluoro-2-(((S)-1-(4-fluoropiperidin-1-yl)propan-2-yl)oxy)quinazolin-4-yl)-3,8-diazabicyclo[3.2.1]octane-8-carboxylate (30 mg, 0.037 mmol) in dichloromethane (1.5 mL) was added trifluoroacetic acid (1.0 mL) and it was stirred at room temperature for 3 h. Solvent was evaporated and the residue was adjusted to pH=11 with aq. Potassium carbonate. The mixture was diluted with dichloromethane:methanol=10:1 solvent and water. Combined organic layer was dried over sodium sulfate and evaporated. The residue was lyophilized to give the title product (10 mg, 44%). ¹H NMR (400 MHz, CD₃OD) δ 7.85 (s, 1H), 7.24-7.18 (m, 1H), 7.01-6.94 (m, 1H), 5.58-5.49 (m, 1H), 4.67-4.35 (m, 3H), 3.68-3.53 (m, 4H), 2.81-2.66 (m, 3H), 2.57-2.46 (m, 3H), 1.90-1.68 (m, 8H), 1.38-1.37 (d, J=1.4 Hz, 3H). MS (ESI, m/e) [M+H]⁺ 618.5.

### Example 26: 1-((2S)-2-((7-(2-amino-7-fluorobenzo[d]thiazol-4-yl)-4-(3,8-diazabicyclo[3.2.1]octan-3-yl)-6-chloro-8-fluoroquinazolin-2-yl)oxy)propyl)piperidine-4-carbonitrile

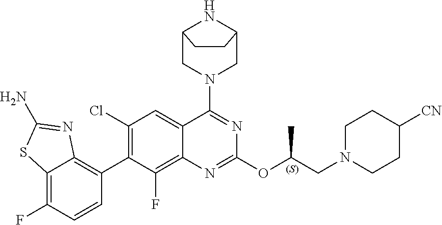

### Step 1: (S)-1-(2-hydroxypropyl)piperidine-4-carbonitrile

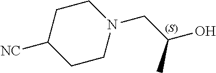

To a solution of piperidine-4-carbonitrile (220 mg, 2 mmol) in water (15 mL) was added (S)-2-methyloxirane (93 mg, 1.6 mmol), and the mixture was stirred at room temperature for 2 hrs. The resulting mixture was extracted with DCM (30 mL×3), and the combined organic layer was concentrated to give a residue (230 mg) which was used in next step without further purification. MS (ESI, m/e) [M+1]⁺169.1.

### Step 2: tert-butyl 3-(7-(2-((tert-butoxycarbonyl)amino)-7-fluorobenzo[d]thiazol-4-yl)-6-chloro-2-(((S)-1-(4-cyanopiperidin-1-yl)propan-2-yl)oxy)-8-fluoroquinazolin-4-yl)-3,8-diazabicyclo[3.2.1]octane-8-carboxylate

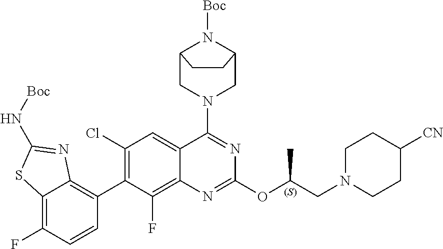

NaH (14 mg, 0.089 mmol) was added to a solution of (S)-1-(2-hydroxypropyl)piperidine-4-carbonitrile (30 mg, 0.178 mmol) in THF (10 mL), and the mixture was stirred at 0° C. for 0.5 h. Then a solution of tert-butyl 3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (107 mg, 0.16 mmol) in THF (10 mL) was added in, and the mixture was stirred at room temperature for 1 h. The resulting mixture was quenched with ice water (10 ml) and extracted with EtOAc (30 mL×3), and the combined organic layer was concentrated to give a residue which was purified by chromatography column on silica (eluting with DCM/MeOH=10/1) to give the title product (50 mg, 38%). MS (ESI, m/e) [M+1]⁺824.3.

### Step 3: 1-((2S)-2-((7-(2-amino-7-fluorobenzo[d]thiazol-4-yl)-4-(3,8-diazabicyclo[3.2.1]octan-3-yl)-6-chloro-8-fluoroquinazolin-2-yl)oxy)propyl)piperidine-4-carbonitrile

To a solution of tert-butyl 3-(7-(2-((tert-butoxycarbonyl)amino)-7-fluorobenzo[d]thiazol-4-yl)-6-chloro-2-(((S)-1-(4-cyanopiperidin-1-yl)propan-2-yl)oxy)-8-fluoroquinazolin-4-yl)-3,8-diazabicyclo[3.2.1]octane-8-carboxylate (50 mg, 0.025 mmol) in DCM (8 mL) was added TFA (3 mL), and the mixture was stirred at room temperature for 1 h. The resulting mixture was concentrated at room temperature and pH was adjusted to 7 with Na₂CO₃, and the organic layer was concentrated to give a residue which was further purified by Prep-HPLC to give the title product (26 mg, 68%). ¹H NMR (500 MHz, CD₃OD) δ 7.86 (s, 1H), 7.20 (m, 1H), 6.98 (t, J=8.8 Hz, 1H), 5.52 (m, 1H), 4.51 (t, J=12.9 Hz, 1H), 4.41 (t, J=12.0 Hz, 1H), 3.63 (m, 4H), 2.87-2.70 (m, 4H), 2.57-2.38 (m, 3H), 1.88 (m, 4H), 1.83-1.67 (m, 3H), 1.37 (m, 3H), 1.31 (m, 1H). MS (ESI, m/e) [M+1]⁺624.2.

### Example 27: 4-(4-(3,8-diazabicyclo[3.2.1]octan-3-yl)-6-chloro-8-fluoro-2-(((2S)-1-(2-methylpiperidin-1-yl)propan-2-yl)oxy)quinazolin-7-yl)-7-fluorobenzo[d]thiazol-2-amine

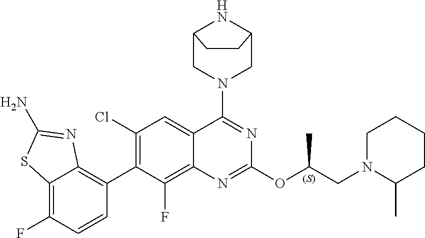

### Step 1: (2S)-1-(2-methylpiperidin-1-yl)propan-2-ol

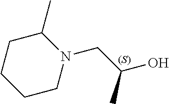

To a solution of 2-methylpiperidine (198 mg, 2 mmol) in water (10 mL) was added (S)-2-methyloxirane (93 mg, 1.6 mmol), and the mixture was stirred at room temperature for 2 hrs. The resulting mixture was extracted with DCM (30 mL×3), and the combined organic layer was concentrated to get the crude product which was used in next step directly. MS (ESI, m/e) [M+1]⁺158.1.

### Step 2: tert-butyl 3-(7-(2-((tert-butoxycarbonyl)amino)-7-fluorobenzo[d]thiazol-4-yl)-6-chloro-8-fluoro-2-(((2S)-1-(2-methylpiperidin-1-yl)propan-2-yl)oxy)quinazolin-4-yl)-3,8-diazabicyclo[3.2.1]octane-8-carboxylate

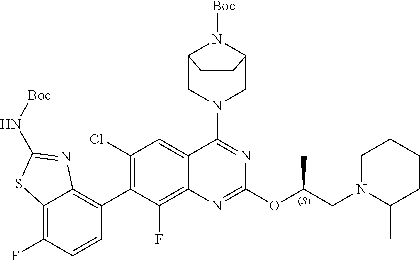

NaH (68 mg, 1.7 mmol) was added to a solution of (2S)-1-(2-methylpiperidin-1-yl)propan-2-ol (104 mg, 0.213 mmol) in THF (15 mL), and the mixture was stirred at 0° C. for 0.5 h. Then tert-butyl 3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (133 mg, 0.85 mmol) in THF (10 mL) was added in and the mixture was stirred at room temperature for 1 h. The resulting mixture was quenched with ice water (10 ml) and extracted with EtOAc (30 mL×3), and the combined organic layer was concentrated and the crude product was purified by chromatography column on silica (eluting with DCM/MeOH=10/1) to give the title product (115 mg, 88%). MS (ESI, m/e) [M+1]⁺814.3.

### Step 3: 4-(4-(3,8-diazabicyclo[3.2.1]octan-3-yl)-6-chloro-8-fluoro-2-(((2S)-1-(2-methylpiperidin-1-yl)propan-2-yl)oxy)quinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-6-chloro-8-fluoro-2-(((2S)-1-(2-methylpiperidin-1-yl)propan-2-yl)oxy)quinazolin-4-yl)-3,8-diazabicyclo [3.2.1]octane-8-carboxylate (115 mg, 0.14 mmol) in DCM (10 mL) was added TFA (3 mL), and the mixture was stirred at room temperature for 1 h. The resulting mixture was concentrated and pH was adjusted to 7 with Na₂CO₃, and the organic layer was concentrated to give a residue which was further purified by Prep-HPLC to give title product (35.5 mg, 41%). ¹H NMR (500 MHz, CD₃OD) δ 7.89 (s, 1H), 7.21 (dt, J=8.4, 5.2 Hz, 1H), 6.99 (t, J=8.8 Hz, 1H), 5.56 (m, 1H), 4.48 (m, 2H), 3.71-3.56 (m, 4H), 3.34 (m, 1H), 2.93 (m, 1H), 2.74 (m, 1H), 1.78 (m, 9H), 1.45-1.39 (m, 4H), 1.31 (m, 1H), 1.28 (m, 4H). MS (ESI, m/e) [M+1]⁺614.5.

### Example 28: (3R,5S)-5-(((7-(2-amino-7-fluorobenzo[d]thiazol-4-yl)-4-(3,8-diazabicyclo[3.2.1]octan-3-yl)-6-chloro-8-fluoroquinazolin-2-yl)oxy)methyl)-1-methylpyrrolidin-3-ol

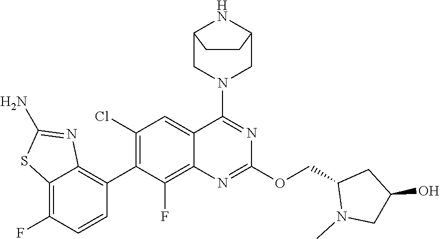 [SMILES: CN1C[C@H](O)C[C@H]1COc1nc(N2CC3CCC(C2)N3)c2cc(Cl)c(-c3ccc(F)c4sc(N)nc34)c(F)c2n1]

### Step 1: tert-butyl 3-(7-(2-((tert-butoxycarbonyl)amino)-7-fluorobenzo[d]thiazol-4-yl)-6-chloro-8-fluoro-2-(((2S,4R)-4-hydroxy-1-methylpyrrolidin-2-yl)methoxy)quinazolin-4-yl)-3,8-diazabicyclo[3.2.1]octane-8-carboxylate

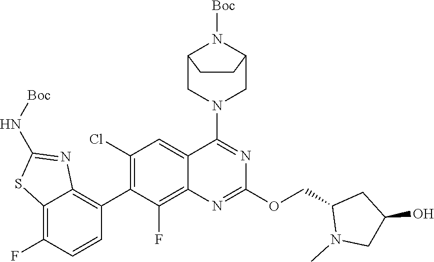 [SMILES: CN1C[C@H](O)C[C@H]1COc1nc(N2CC3CCC(C2)N3C(=O)OC(C)(C)C)c2cc(Cl)c(-c3ccc(F)c4sc(NC(=O)OC(C)(C)C)nc34)c(F)c2n1]

A solution of tert-butyl 3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (2.01 g, 2.98 mmol) in THF (500 mL) was cooled to 0° C., NaH (0.26 g, 6.55 mmol) was added, and the mixture was stirred for 3 hours at 0° C. room temperature. The cooled resulting mixture was diluted with EtOAc (300 mL) and saturated NH₄Cl solution (200 ML). Aqueous layer was extracted with EtOAc, and the combined organic layer was washed with brine, dried over Na₂SO₄, concentrated. The crude product was purified by flash to give the title compound (1.11 g). MS (ESI, m/e) [M+1]⁺788.9.

### Step 2: (3R,5S)-5-(((7-(2-amino-7-fluorobenzo[d]thiazol-4-yl)-4-(3,8-diazabicyclo [3.2.1]octan-3-yl)-6-chloro-8-fluoroquinazolin-2-yl)oxy)methyl)-1-methylpyrrolidin-3-ol

To a solution of tert-butyl 3-(7-(2-((tert-butoxycarbonyl)amino)-7-fluorobenzo[d]thiazol-4-yl)-6-chloro-8-fluoro-2-(((2S,4R)-4-hydroxy-1-methylpyrrolidin-2-yl)methoxy)quinazolin-4-yl)-3,8-diazabicyclo[3.2.1]octane-8-carboxylate (50 mg, 0.006 mmol) in DCM (10 mL) was added BF₃Et₂O (2 drops), and the mixture was stirred at room temperature for 4 hrs. The reaction mixture was quenched with 5 mL of MeOH, then concentrated to dryness. Crude product was purified by Prep-HPLC to give title compound (25 mg). ¹H NMR (500 MHz, DMSO-d₆) δ 9.33 (s, 1H), 7.90 (s, 3H), 7.21 (dd, J=8.5, 8.0 Hz, 1H), 7.07 (t, J=8.8 Hz, 1H), 5.05 (s, 1H), 4.44 (d, J=12.1 Hz, 4H), 4.25 (s, 1H), 4.11 (s, 2H), 3.73-3.54 (m, 3H), 3.24-3.07 (m, 1H), 2.56 (s, 3H), 1.91 (s, 6H). MS (ESI, m/e) [M+1]⁺588.1.

### Example 29: 4-(4-(3,8-diazabicyclo[3.2.1]octan-3-yl)-6-chloro-8-fluoro-2-(((S)-1-morpholinopropan-2-yl)oxy)quinazolin-7-yl)-7-fluorobenzo[d]thiazol-2-amine

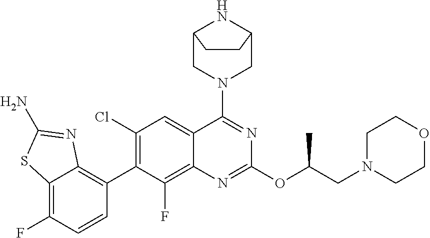 [SMILES: C[C@@H](CN1CCOCC1)Oc1nc(N2CC3CCC(C2)N3)c2cc(Cl)c(-c3ccc(F)c4sc(N)nc34)c(F)c2n1]

### Step 1: (S)-1-morpholinopropan-2-ol

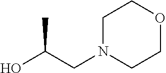

To a mixture of (S)-2-methyloxirane (580 mg, 10.0 mmol) in H₂O (5 mL) was added 1-methylpiperazine (870 mg, 10.0 mmol), The mixture was stirred for 16 hrs at room temperature. The reaction mixture was extracted with DCM/MeOH (v/v=10/1), and the organic layer was combined, dried over sodium sulfate and evaporated to give the crude product which was used directly in next step. MS (ESI, m/e) [M+1]⁺146.

### Step 2: tert-butyl 3-(7-(2-((tert-butoxycarbonyl)amino)-7-fluorobenzo[d]thiazol-4-yl)-6-chloro-8-fluoro-2-(((S)-1-morpholinopropan-2-yl)oxy)quinazolin-4-yl)-3,8-diazabicyclo[3.2.1]octane-8-carboxylate

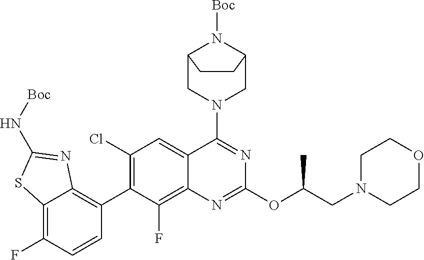 [SMILES: C[C@@H](CN1CCOCC1)Oc1nc(N2CC3CCC(C2)N3C(=O)OC(C)(C)C)c2cc(Cl)c(-c3ccc(F)c4sc(NC(=O)OC(C)(C)C)nc34)c(F)c2n1]

To a solution of (S)-1-morpholinopropan-2-ol (21 mg, 0.148 mmol) in THF (3 mL) was added NaH (9 mg, 0.222 mmol), stirred at room temperature for 0.5 hour, then added tert-butyl 3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (100 mg, 0.148 mmol) in THF (0.5 mL), then the mixture was stirred for more 4 hrs at room temperature. The reaction mixture was diluted with EtOAc and water, extracted with EtOAc twice, and the organic layer was combined, dried over sodium sulfate and evaporated. The residue was purified by chromatography column on silica (eluting with MeOH/DCM=5-7%) to give the title product (60 mg, 50%). MS (ESI, m/e) [M+1]⁺802.

### Step 3: 4-(4-(3,8-diazabicyclo[3.2.1]octan-3-yl)-6-chloro-8-fluoro-2-(((S)-1-morpholinopropan-2-yl)oxy)quinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-6-chloro-8-fluoro-2-(((S)-1-morpholinopropan-2-yl)oxy)quinazolin-4-yl)-3,8-diazabicyclo[3.2.1]octane-8-carboxylate (60 mg, 0.075 mmol) in EtOAc (20 mL) was added HCl/dioxane (2 mL, 4 M) and stirred for 4 hrs at room temperature. Then the resulting mixture was filtered to give the crude product. The crude product was mixed with water, and the pH was adjusted to 9 and extracted with DCM/MeOH (v=10/1). Combined organic layer was concentrated and the residue was purified by chromatography column on silica (eluting with MeOH/DCM=25%) to give the title product (19.6 mg, 43%). ¹H NMR (500 MHz, CD₃OD) δ 7.86 (s, 1H), 7.25-7.17 (m, 1H), 6.98 (t, J=8.8 Hz, 1H), 5.60-5.49 (m, 1H), 4.56-4.35 (m, 2H), 3.70-3.53 (m, 9H), 2.82-2.66 (m, 1H), 2.66-2.47 (m, 5H), 1.93-1.74 (m, 4H), 1.38 (d, J=6.3 Hz, 3H). MS (ESI, m/e) [M+H]⁺ 602.3.

### Example 30: 4-(4-(3,8-diazabicyclo[3.2.1]octan-8-yl)-6-chloro-8-fluoro-2-(((S)-1-(4-fluoropiperidin-1-yl)propan-2-yl)oxy)quinazolin-7-yl)-7-fluorobenzo[d]thiazol-2-amine

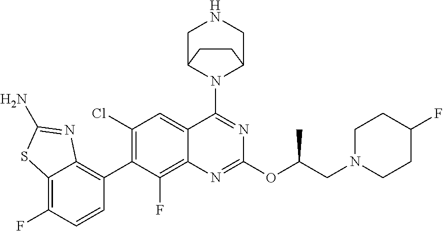 [SMILES: C[C@@H](CN1CCC(F)CC1)Oc1nc(N2C3CCC2CNC3)c2cc(Cl)c(-c3ccc(F)c4sc(N)nc34)c(F)c2n1]

### Step 1: tert-butyl 8-(7-(2-((tert-butoxycarbonyl)amino)-7-fluorobenzo[d]thiazol-4-yl)-6-chloro-8-fluoro-2-(((S)-1-(4-fluoropiperidin-1-yl)propan-2-yl)oxy)quinazolin-4-yl)-3,8-diazabicyclo[3.2.1]octane-3-carboxylate

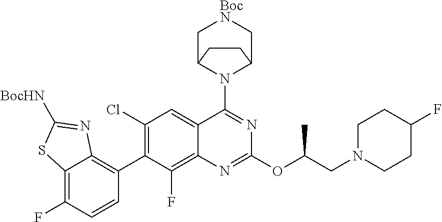 [SMILES: C[C@@H](CN1CCC(F)CC1)Oc1nc(N2C3CCC2CN(C(=O)OC(C)(C)C)C3)c2cc(Cl)c(-c3ccc(F)c4sc(NC(=O)OC(C)(C)C)nc34)c(F)c2n1]

To a solution of (S)-1-(4-fluoropiperidin-1-yl)propan-2-ol (100 mg, 0.62 mmol) in THF (3 mL) was added sodium hydride (60%) (36 mg, 0.9 mmol) at 0° C. The mixture was stirred at room temperature for 1 h. Then tert-butyl-8-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-3-carboxylate (105 mg, 0.16 mmol) in 1 mL tetrahydrofuran was added dropwise and it was stirred at room temperature for another 1 h. The mixture was diluted with dichloromethane and water. Combined organic layer was dried over sodium sulfate and evaporated. The residue was purified by preparative thin layer chromatography to give the title product (30 mg, 24%). MS (ESI, m/e) [M+1]⁺818.4.

### Step 3: 4-(4-(3,8-diazabicyclo[3.2.1]octan-8-yl)-6-chloro-8-fluoro-2-(((S)-1-(4-fluoropiperidin-1-yl)propan-2-yl)oxy)quinazolin-7-yl)-7-fluorobenzo[d]thiazol-2-amine

To a solution of tert-butyl 8-(7-(2-((tert-butoxycarbonyl)amino)-7-fluorobenzo[d]thiazol-4-yl)-6-chloro-8-fluoro-2-(((S)-1-(4-fluoropiperidin-1-yl)propan-2-yl)oxy)quinazolin-4-yl)-3,8-diazabicyclo[3.2.1]octane-3-carboxylate (30 mg, 0.037 mmol) in dichloromethane (1.5 mL) was added trifluoroacetic acid (1.0 mL) and it was stirred at room temperature for 3 h. Solvent was evaporated and the residue was adjusted to pH=11 with aq. Potassium carbonate. The mixture was diluted with dichloromethane:methanol=10:1 solvent and water. Combined organic layer was dried over sodium sulfate and evaporated. The residue was lyophilized to give the title product (5 mg, 22%). ¹H NMR (400 MHz, CD₃OD) δ 7.98-7.97 (m, 1H), 7.24-7.18 (m, 1H), 7.01-6.94 (m, 1H), 5.70-5.64 (m, 1H), 5.10-5.04 (m, 1H), 3.95-3.72 (m, 2H), 3.69-3.54 (m, 3H), 3.52-3.36 (m, 6H), 3.30-3.16 (m, 3H), 2.30-1.94 (m, 6H), 1.47-1.46 (d, J=1.4 Hz, 3H). MS (ESI, m/e) [M+H]⁺ 618.4.

### Example 31: 4-(4-(3,8-diazabicyclo[3.2.1]octan-3-yl)-6-chloro-8-fluoro-2-(((S)-1-(3-fluoroazetidin-1-yl)propan-2-yl)oxy)quinazolin-7-yl)-7-fluorobenzo[d]thiazol-2-amine

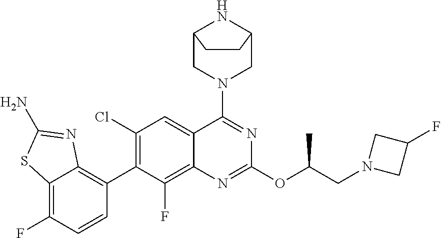 [SMILES: C[C@@H](CN1CC(F)C1)Oc1nc(N2CC3CCC(C2)N3)c2cc(Cl)c(-c3ccc(F)c4sc(N)nc34)c(F)c2n1]

### Step 1: (S)-1-(3-fluoroazetidin-1-yl)propan-2-ol

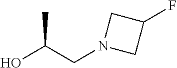

A solution of 3-fluoroazetidine hydrogen chloride (625 mg, 5.63 mmol) in 5 mL water was adjusted pH to 8 with sodium bicarbonate. (S)-2-methyloxirane (300 mg, 5.00 mmol) was added at 0° C. The mixture was stirred at room temperature for 16 hrs. Then it was diluted with dichloromethane. Combined organic layer was dried over sodium sulfate and evaporated to give crude product which was used directly. MS (ESI, m/e) [M+1]⁺134.2.

### Step 2: tert-butyl 3-(7-(2-((tert-butoxycarbonyl)amino)-7-fluorobenzo[d]thiazol-4-yl)-6-chloro-8-fluoro-2-(((S)-1-(3-fluoroazetidin-1-yl)propan-2-yl)oxy)quinazolin-4-yl)-3,8-diazabicyclo[3.2.1]octane-8-carboxylate

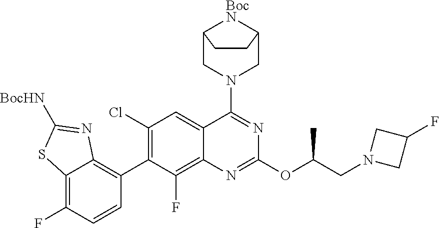 [SMILES: C[C@@H](CN1CC(F)C1)Oc1nc(N2CC3CCC(C2)N3C(=O)OC(C)(C)C)c2cc(Cl)c(-c3ccc(F)c4sc(NC(=O)OC(C)(C)C)nc34)c(F)c2n1]

To a solution of (S)-1-(3-fluoroazetidin-1-yl)propan-2-ol (80 mg, 0.60 mmol) in THF (2 mL) was added sodium hydride (60%) (36 mg, 0.9 mmol) at 0° C. The mixture was stirred at room temperature for 1 h. Then tert-butyl 3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (100 mg, 0.15 mmol) in 1 mL tetrahydrofuran was added dropwise and it was stirred at room temperature for another 1 h. And it was diluted with dichloromethane and water. Combined organic layer was dried over sodium sulfate and evaporated. The residue was purified by preparative thin layer chromatography to give the title product (30 mg, 26%). MS (ESI, m/e) [M+1]⁺790.4.

### Step 3: 4-(4-(3,8-diazabicyclo[3.2.1]octan-3-yl)-6-chloro-8-fluoro-2-(((S)-1-(3-fluoroazetidin-1-yl)propan-2-yl)oxy)quinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-6-chloro-8-fluoro-2-(((S)-1-(3-fluoroazetidin-1-yl)propan-2-yl)oxy)quinazolin-4-yl)-3,8-diazabicyclo[3.2.1]octane-8-carboxylate (30 mg, 0.038 mmol) in dichloromethane (1.5 mL) was added trifluoroacetic acid (1.0 mL) and it was stirred at room temperature for 3 hrs. Solvent was evaporated and the residue was adjusted to pH=11 with aq. Potassium carbonate and it was diluted with dichloromethane:methanol=10:1 solvent and water. Combined organic layer was dried over sodium sulfate and evaporated. The residue was lyophilized to give the title product (12 mg, 53%). ¹H NMR (400 MHz, CD₃OD) δ 7.86 (s, 1H), 7.24-7.18 (m, 1H), 7.01-6.94 (m, 1H), 5.39-5.29 (m, 1H), 5.17-5.00 (m, 1H), 4.57-4.35 (m, 2H), 3.78-3.53 (m, 5H), 3.44-3.36 (m, 1H), 3.35-3.30 (m, 2H), 2.90-2.76 (m, 2H), 1.90-1.68 (m, 4H), 1.36-1.35 (d, J=1.4 Hz, 3H). MS (ESI, m/e) [M+H]⁺ 590.4.

### Example 32: 4-(4-(3,8-diazabicyclo[3.2.1]octan-3-yl)-6-chloro-2-(((S)-1-(3,3-difluoroazetidin-1-yl)propan-2-yl)oxy)-8-fluoroquinazolin-7-yl)-7-fluorobenzo[d]thiazol-2-amine

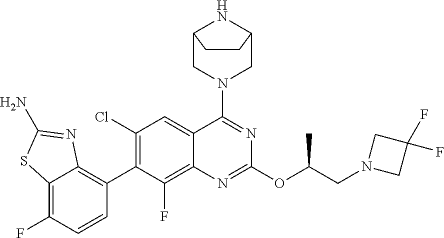 [SMILES: C[C@@H](CN1CC(F)(F)C1)Oc1nc(N2CC3CCC(C2)N3)c2cc(Cl)c(-c3ccc(F)c4sc(N)nc34)c(F)c2n1]

### Step 1: (5)-1-(3,3-difluoroazetidin-1-yl)propan-2-ol

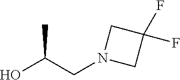

A solution of 3,3-difluoroazetidine hydrogen chloride (600 mg, 4.61 mmol) in 5 mL water was adjusted pH to 8 with sodium bicarbonate and (S)-2-methyloxirane (240 mg, 4.00 mmol) was added at 0° C. The mixture was stirred at room temperature for 16 hrs. Then it was diluted with dichloromethane. Combined organic layer was dried over sodium sulfate and evaporated to give crude product which was used directly. MS (ESI, m/e) [M+1]⁺152.2.

### Step 2: tert-butyl 3-(7-(2-((tert-butoxycarbonyl)amino)-7-fluorobenzo[d]thiazol-4-yl)-6-chloro-2-(((S)-1-(3,3-difluoroazetidin-1-yl)propan-2-yl)oxy)-8-fluoroquinazolin-4-yl)-3,8-diazabicyclo[3.2.1]octane-8-carboxylate

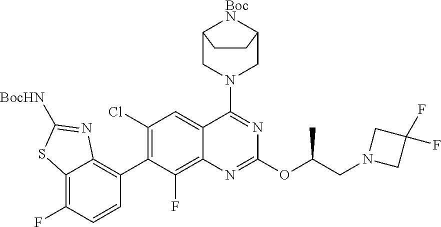 [SMILES: C[C@@H](CN1CC(F)(F)C1)Oc1nc(N2CC3CCC(C2)N3C(=O)OC(C)(C)C)c2cc(Cl)c(-c3ccc(F)c4sc(NC(=O)OC(C)(C)C)nc34)c(F)c2n1]

To a solution of (S)-1-(3,3-difluoroazetidin-1-yl)propan-2-ol (100 mg, 0.66 mmol) in 3 mL tetrahydrofuran was added sodium hydride (60%) (36 mg, 0.9 mmol) at 0° C. The mixture was stirred at room temperature for 1 h. Then tert-butyl 3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (100 mg, 0.15 mmol) in 1 mL tetrahydrofuran was added dropwise and it was stirred at room temperature for another 1 h. The mixture was diluted with dichloromethane and water. Combined organic layer was dried over sodium sulfate and evaporated. The residue was purified by preparative thin layer chromatography to give the title product (30 mg, 25%). MS (ESI, m/e) [M+1]⁺808.4.

### Step 3: 4-(4-(3,8-diazabicyclo[3.2.1]octan-3-yl)-6-chloro-2-(((S)-1-(3,3-difluoroazetidin-1-yl)propan-2-yl)oxy)-8-fluoroquinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-6-chloro-2-(((S)-1-(3,3-difluoroazetidin-1-yl)propan-2-yl)oxy)-8-fluoroquinazolin-4-yl)-3,8-diazabicyclo[3.2.1]octane-8-carboxylate (30 mg, 0.037 mmol) in dichloromethane (1.5 mL) was added trifluoroacetic acid (1.0 mL) and it was stirred at room temperature for 3 h. Solvent was evaporated and the residue was adjusted to pH=11 with aq. Potassium carbonate. The mixture was diluted with dichloromethane:methanol=10:1 solvent and water. Combined organic layer was dried over sodium sulfate and evaporated. The residue was lyophilized to give the title product (7 mg, 31%). ¹H NMR (400 MHz, CD₃OD) δ 7.86 (s, 1H), 7.24-7.18 (m, 1H), 7.01-6.94 (m, 1H), 5.40-5.31 (m, 1H), 4.57-4.35 (m, 2H), 3.81-3.53 (m, 8H), 2.96-2.80 (m, 2H), 1.91-1.70 (m, 4H), 1.37-1.36 (d, J=1.4 Hz, 3H). MS (ESI, m/e) [M+H]⁺ 608.4.

### Example 33: 4-(4-(3,8-diazabicyclo[3.2.1]octan-3-yl)-6-chloro-2-(((S)-1-(3,6-dihydropyridin-1(2H)-yl)propan-2-yl)oxy)-8-fluoroquinazolin-7-yl)-7-fluorobenzo[d]thiazol-2-amine

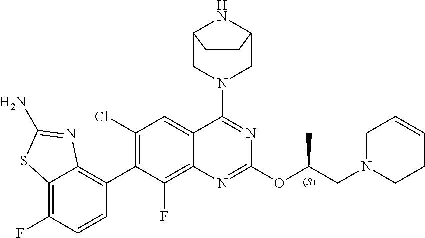

### Step 1: (S)-1-(3,6-dihydropyridin-1(2H)-yl)propan-2-ol

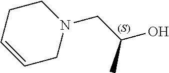

To a solution of 1,2,3,6-tetrahydropyridine (166 mg, 2 mmol) in water (10 mL) was added (S)-2-methyloxirane (93 mg, 1.6 mmol), and the mixture was stirred at room temperature for 2 h. The resulting mixture was extracted with DCM (30 mL×3), and the combined organic layer was concentrated to get the crude product which was used next step without future purification. MS (ESI, m/e) [M+1]⁺142.1.

### Step 2: tert-butyl 3-(7-(2-((tert-butoxycarbonyl)amino)-7-fluorobenzo[d]thiazol-4-yl)-6-chloro-2-(((S)-1-(3,6-dihydropyridin-1(2H)-yl)propan-2-yl)oxy)-8-fluoroquinazolin-4-yl)-3,8-diazabicyclo[3.2.1]octane-8-carboxylate

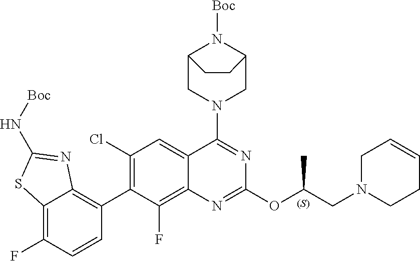

NaH (60 mg, 1.52 mmol) was added to a solution of (S)-1-(3,6-dihydropyridin-1(2H)-yl)propan-2-ol (107 mg, 0.76 mmol) in THF (15 mL), and the mixture was stirred at 0° C. for 0.5 h. Then a solution of tert-butyl 3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (460 mg, 0.68 mmol) in THF (20 mL) was added in, and the mixture was stirred at room temperature for 1 h. The resulting mixture was quenched with ice water (10 mL) and extracted with EtOAc (30 mL×3), and the combined organic layer was concentrated and purified by chromatography column on silica (eluting with DCM/MeOH=10/1) to give the title product (150 mg, 28%). MS (ESI, m/e) [M+1]⁺798.3.

### Step 3: 4-(4-(3,8-diazabicyclo[3.2.1]octan-3-yl)-6-chloro-2-(((S)-1-(3,6-dihydropyridin-1(2H)-yl)propan-2-yl)oxy)-8-fluoroquinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-6-chloro-2-(((S)-1-(3,6-dihydropyridin-1(2H)-yl)propan-2-yl)oxy)-8-fluoroquinazolin-4-yl)-3,8-diazabicyclo[3.2.1]octane-8-carboxylate (150 mg, 0.19 mmol) in DCM (10 mL) was added TFA (3 mL), and the mixture was stirred at room temperature for 1 h. The resulting mixture was concentrated at room temperature and pH was adjusted to 7 with Na₂CO₃, and the organic layer was concentrated to give a residue which was further purified by Prep-HPLC to give title product (1.02 mg, 1%). ¹H NMR (500 MHz, CD₃OD) δ 7.90 (s, 1H), 7.21 (dd, J=14.0, 6.1 Hz, 1H), 7.00 (t, J=8.7 Hz, 1H), 5.84 (m, 1H), 5.67 (m, 2H), 4.56 (m, 3H), 4.02 (m, 2H), 3.81-3.71 (m, 2H), 3.55 (m, 2H), 3.15-3.07 (m, 3H), 2.31 (m, 2H), 2.04 (m, 4H), 1.45 (m, 3H). MS (ESI, m/e) [M+1]⁺598.4.

### Example 34: 4-(4-(3,8-diazabicyclo[3.2.1]octan-3-yl)-6-chloro-8-fluoro-2-(((S)-1-(4-methylpiperazin-1-yl)propan-2-yl)oxy)quinazolin-7-yl)-7-fluorobenzo[d]thiazol-2-amine

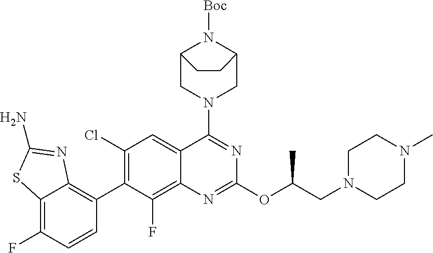 [SMILES: C[C@@H](CN1CCN(C)CC1)Oc1nc(N2CC3CCC(C2)N3C(=O)OC(C)(C)C)c2cc(Cl)c(-c3ccc(F)c4sc(N)nc34)c(F)c2n1]

### Step 1: tert-butyl 3-(7-(2-((tert-butoxycarbonyl)amino)-7-fluorobenzo[d]thiazol-4-yl)-6-chloro-8-fluoro-2-(((S)-1-(4-methylpiperazin-1-yl)propan-2-yl)oxy)quinazolin-4-yl)-3,8-diazabicyclo[3.2.1]octane-8-carboxylate

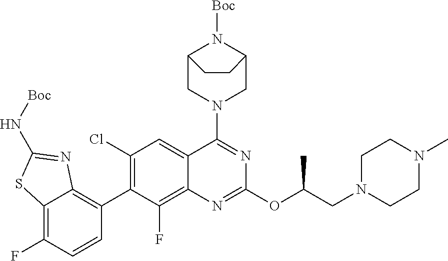 [SMILES: C[C@@H](CN1CCN(C)CC1)Oc1nc(N2CC3CCC(C2)N3C(=O)OC(C)(C)C)c2cc(Cl)c(-c3ccc(F)c4sc(NC(=O)OC(C)(C)C)nc34)c(F)c2n1]

To a solution of (S)-1-(4-methylpiperazin-1-yl)propan-2-ol (35 mg, 0.223 mmol) in THF (3 mL) was added NaH (10 mg, 0.245 mmol), and the mixture was stirred at room temperature for 0.5 h. then tert-butyl 3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (150 mg, 0.223 mmol) in THF (0.5 mL) was added, and the mixture was stirred for another 4 hrs at room temperature. The reaction mixture was diluted with EtOAc and water, extracted with EtOAc twice, and the organic layer was combined, dried over sodium sulfate and evaporated. The residue was purified by chromatography column on silica (eluting with MeOH/DCM=5-7%) to give the title product (50 mg, 27%). MS (ESI, m/e) [M+1]⁺815.

### Step 2: 4-(4-(3,8-diazabicyclo[3.2.1]octan-3-yl)-6-chloro-8-fluoro-2-(((S)-1-(4-methylpiperazin-1-yl)propan-2-yl)oxy)quinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-6-chloro-8-fluoro-2-(((S)-1-(4-methylpiperazin-1-yl)propan-2-yl)oxy)quinazolin-4-yl)-3,8-diazabicyclo[3.2.1]octane-8-carboxylate (50 mg, 0.061 mmol) in EtOAc (20 mL) was added HCl/dioxane (2 mL, 4 M) and the mixture was stirred for 4 hrs at room temperature. Then the resulting mixture was filtered to give the crude product. The crude product was mixed with water, and the pH was adjusted to 9 and extracted with DCM/MeOH (v=10/1). Combined organic layer was concentrated and the residue was purified by chromatography column on silica (eluting with MeOH/DCM=20%) to give the title product (14.8 mg, 39%). ¹H NMR (400 MHz, DMSO-d₆) δ 7.91 (s, 2H), 7.84 (s, 1H), 7.26-7.16 (m, 1H), 7.06 (t, J=8.8 Hz, 1H), 5.39-5.29 (m, 1H), 4.29 (t, J=10.0 Hz, 2H), 3.69 (s, 2H), 3.61-3.52 (m, 2H), 2.63 (t, J=9.5 Hz, 1H), 2.28 (s, 3H), 2.12 (d, J=7.0 Hz, 3H), 1.98 (s, 1H), 1.71 (s, 3H), 1.29 (d, J=6.2 Hz, 3H), 1.24 (s, 6H), 0.85 (t, J=6.7 Hz, 1H). MS (ESI, m/e) [M+H]⁺ 615.3.

### Example 35: 6-(4-(3,8-diazabicyclo[3.2.1]octan-3-yl)-6-chloro-8-fluoro-2-(((S)-1-(piperidin-1-yl)propan-2-yl)oxy)quinazolin-7-yl)-4-methyl-5-(trifluoromethyl)pyridin-2-amine

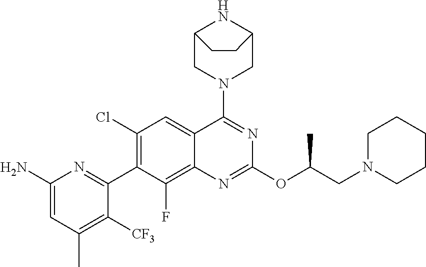 [SMILES: Cc1cc(N)nc(-c2c(Cl)cc3c(N4CC5CCC(C4)N5)nc(O[C@@H](C)CN4CCCCC4)nc3c2F)c1C(F)(F)F]

Synthetic Route:

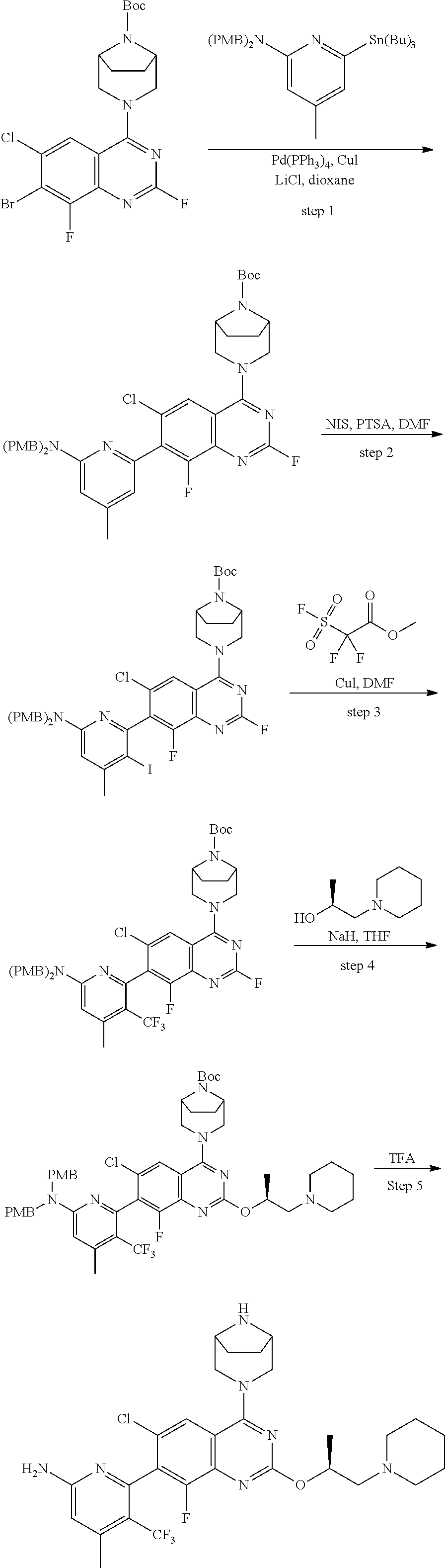

### Step 1: tert-butyl 3-(7-(6-(bis(4-methoxybenzyl)amino)-4-methylpyridin-2-yl)-6-chloro-2,8-difluoroquinazolin-4-yl)-3,8-diazabicyclo[3.2.1]octane-8-carboxylate

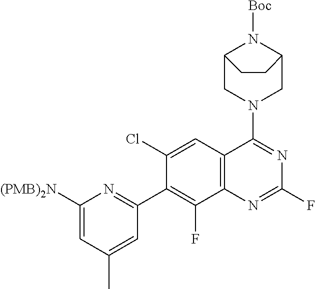

A mixture of tert-butyl 3-(7-bromo-6-chloro-2,8-difluoroquinazolin-4-yl)-3,8-diazabicyclo[3.2.1]octane-8-carboxylate (4.50 g, 9.19 mmol), dioxane (120 mL), N,N-bis[(4-methoxyphenyl)methyl]-4-methyl-6-(tributylstannyl)pyridin-2-amine (8.79 g, 13.78 mmol), Pd(PPh₃)₄ (5.31 g, 4.59 mmol), CuI (997.5 mg, 5.24 mmol), LiCl (973.8 mg, 22.97 mmol) was stirred overnight at 100° C. Upon completion, the resulting mixture was concentrated under vacuum and the crude product was purified by Prep-HPLC to give the title product (3.5 g, 50%). MS (ESI, m/e) [M+H]⁺ 757.

### Step 2: tert-butyl 3-(7-(6-(bis(4-methoxybenzyl)amino)-3-iodo-4-methylpyridin-2-yl)-6-chloro-2,8-difluoroquinazolin-4-yl)-3,8-diazabicyclo[3.2.1]octane-8-carboxylate

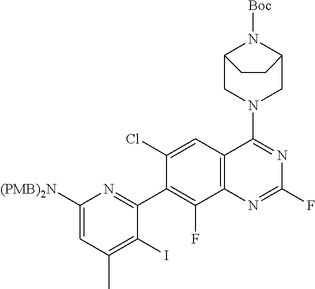

To a solution of tert-butyl 3-(7-(6-(bis(4-methoxybenzyl)amino)-4-methylpyridin-2-yl)-6-chloro-2,8-difluoroquinazolin-4-yl)-3,8-diazabicyclo[3.2.1]octane-8-carboxylate (3.50 g, 4.62 mmol) in DMF (80 mL) was added PTSA (477.5 mg, 2.77 mmol) in portions at room temperature in 1 min. The resulting solution was stirred for 5 min at room temperature. To this was added NIS (2.08 g, 9.244 mmol) and the resulting solution was stirred overnight at room temperature. The resulting solution was diluted with 50 mL of H₂O and extracted with 80 mL of ethyl acetate. The resulting mixture was washed with brine (45 mL×3) and the organic later was concentrated under vacuum. The crude product was purified by Prep-HPLC to give the title product (2.1 g, 51%). MS (ESI, m/e) [M+H]⁺ 883.

### Step 3: tert-butyl 3-(7-(6-(bis(4-methoxybenzyl)amino)-4-methyl-3-(trifluoromethyl)pyridin-2-yl)-6-chloro-2,8-difluoroquinazolin-4-yl)-3,8-diazabicyclo[3.2.1]octane-8-carboxylate

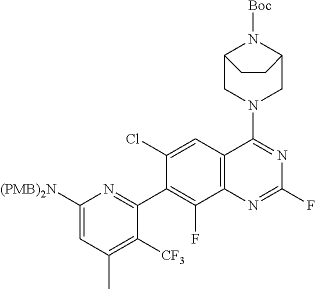

A mixture of tert-butyl 3-(7-(6-(bis(4-methoxybenzyl)amino)-3-iodo-4-methylpyridin-2-yl)-6-chloro-2,8-difluoroquinazolin-4-yl)-3,8-diazabicyclo[3.2.1]octane-8-carboxylate (2.10 g, 2.38 mmol), DMF (90 mL), CuI (1.81 g, 9.51 mmol), methyl 2,2-difluoro-2-(fluorosulfonyl)acetate (4.57 g, 23.78 mmol) was stirred overnight at 90° C. The mixture was then diluted with 60 mL of H₂O and extracted with 90 mL of ethyl acetate. The resulting mixture was washed with brine (45 mL×3) and the organic later was concentrated under vacuum. The crude product was purified by Prep-HPLC to give the title product (1.3 g, 66%). MS (ESI, m/e) [M+H]⁺ 825.3.

### Step 4: tert-butyl 3-(7-(6-(bis(4-methoxybenzyl)amino)-4-methyl-3-(trifluoromethyl)pyridin-2-yl)-6-chloro-8-fluoro-2-(((S)-1-(piperidin-1-yl)propan-2-yl)oxy)quinazolin-4-yl)-3,8-diazabicyclo[3.2.1]octane-8-carboxylate

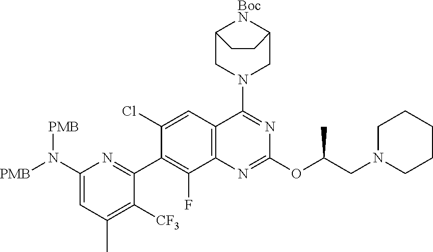

To a solution of (S)-1-(piperidin-1-yl)propan-2-ol (72 mg, 0.50 mmol) in 5 mL tetrahydrofuran was added sodium hydride (60%) (36 mg, 0.90 mmol) at 0° C. The mixture was stirred at room temperature for 1 h. Then tert-butyl 3-(7-(6-(bis(4-methoxybenzyl)amino)-4-methyl-3-(trifluoromethyl)pyridin-2-yl)-6-chloro-2,8-difluoroquinazolin-4-yl)-3,8-diazabicyclo[3.2.1]octane-8-carboxylate (100 mg, 0.12 mmol) in 1 mL tetrahydrofuran was added dropwise and the mixture was stirred at room temperature for another 1 h. The mixture was diluted with dichloromethane and water. Combined organic layer was dried over sodium sulfate and evaporated. The residue was purified by preparative thin layer chromatography to give the title product (70 mg, 49%). MS (ESI, m/e) [M+1]⁺948.6.

### Step 5: 6-(4-(3,8-diazabicyclo[3.2.1]octan-3-yl)-6-chloro-8-fluoro-2-(((S)-1-(piperidin-1-yl)propan-2-yl)oxy)quinazolin-7-yl)-4-methyl-5-(trifluoromethyl)pyridin-2-amine

To a solution of tert-butyl 3-(7-(6-(bis(4-methoxybenzyl)amino)-4-methyl-3-(trifluoromethyl)pyridin-2-yl)-6-chloro-8-fluoro-2-(((S)-1-(piperidin-1-yl)propan-2-yl)oxy)quinazolin-4-yl)-3,8-diazabicyclo[3.2.1]octane-8-carboxylate (40 mg, 0.042 mmol) in trifluoroacetic acid (5 mL) and the mixture was stirred at 40° C. for 24 hrs. Solvent was evaporated and the residue was adjusted to pH=11 with aq. Potassium carbonate. The mixture was diluted with dichloromethane:methanol=10:1 solvent and water. Combined organic layer was dried over sodium sulfate and evaporated. The residue was lyophilized to give the title product (25 mg, 97%). ¹H NMR (500 MHz, CD₃OD) δ 7.83 (s, 1H), 6.60 (s, 1H), 5.62-5.51 (m, 1H), 4.51-4.34 (m, 2H), 3.89-3.52 (m, 7H), 2.96-2.59 (m, 3H), 2.44 (s, 3H), 1.89-1.75 (m, 4H), 1.65-1.52 (m, 4H), 1.50-1.42 (m, 2H), 1.39-1.38 (d, J=1.4 Hz, 3H). MS (ESI, m/e) [M+H]⁺ 608.5.

### Example 36: 4-(4-(3,8-diazabicyclo[3.2.1]octan-8-yl)-6-chloro-8-fluoro-2-(((2S,4R)-4-fluoro-1-methylpyrrolidin-2-yl)methoxy)quinazolin-7-yl)-7-fluorobenzo[d]thiazol-2-amine

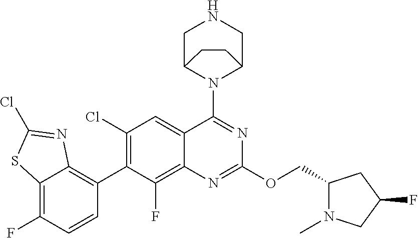 [SMILES: CN1C[C@H](F)C[C@H]1COc1nc(N2C3CCC2CNC3)c2cc(Cl)c(-c3ccc(F)c4sc(Cl)nc34)c(F)c2n1]

Example 36 was prepared by similar procedure as described in Example 19 by replacing (S)-1-(4,4-difluoropiperidin-1-yl)propan-2-ol with ((2S,4R)-4-fluoro-1-methylpyrrolidin-2-yl)methanol. ¹H NMR (400 MHz, CD₃OD) δ 7.98 (s, 1H), 7.22 (ddd, J=8.4, 5.4, 3.1 Hz, 1H), 7.01 (t, J=8.8 Hz, 1H), 5.36 (d, J=53.1 Hz, 1H), 5.14-5.07 (m, 2H), 4.85-4.79 (m, 2H), 4.67-4.61 (m, 1H), 3.91-3.75 (m, 2H), 3.66-3.59 (m, 2H), 3.38-3.34 (m, 2H), 2.96-2.92 (m, 3H), 2.59-2.47 (m, 1H), 2.32-2.24 (m, 3H), 2.13-2.11 (m, 2H). MS (ESI, m/e) [M+1]⁺590.4.

### Example 37: 6-(4-(3,8-diazabicyclo[3.2.1]octan-3-yl)-6-chloro-8-fluoro-2-(((2S,4R)-4-fluoro-1-methylpyrrolidin-2-yl)methoxy)quinazolin-7-yl)-4-methyl-5-(trifluoromethyl)pyridin-2-amine

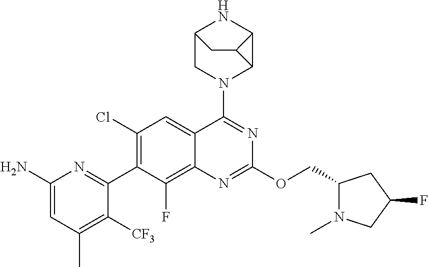 [SMILES: Cc1cc(N)nc(-c2c(Cl)cc3c(N4CC5CC6C(N5)C64)nc(OC[C@@H]4C[C@@H](F)CN4C)nc3c2F)c1C(F)(F)F]

Example 37 was prepared by similar procedure as described in Example 35 by replacing (S)-1-(piperidin-1-yl)propan-2-ol with ((2S,4R)-4-fluoro-1-methylpyrrolidin-2-yl)methanol. ¹H NMR (400 MHz, CD₃OD) δ 7.95 (d, J=1.6 Hz, 1H), 6.68 (s, 1H), 5.50 (d, J=52.4 Hz, 1H), 4.98 (dd, J=13.2, 2.4 Hz, 1H), 4.78-4.64 (m, 3H), 4.26 (s, 3H), 4.09 (d, J=25.8 Hz, 1H), 3.97-3.81 (m, 2H), 3.71 (d, J=19.2 Hz, 1H), 3.21 (s, 3H), 2.70 (m, 1H), 2.48 (q, J=2.2 Hz, 4H), 2.15 (s, 4H). MS (ESI, m/e) [M+1]⁺598.05

### Example 38: 4-(4-(3,8-diazabicyclo[3.2.1]octan-3-yl)-6-chloro-2-(((2S)-1-(2,6-dimethylpiperidin-1-yl)propan-2-yl)oxy-8-fluoroquinazolin-7-yl)-7-fluorobenzo[d]thiazol-2-amine

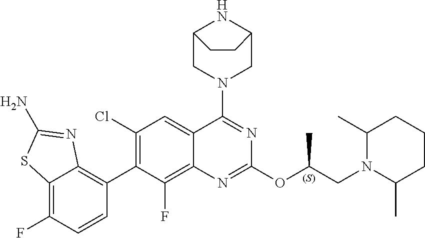

### Step 1: tert-butyl 3-(7-(2-((tert-butoxycarbonyl)amino)-7-fluorobenzo[d]thiazol-4-yl)-6-chloro-2-(((2S)-1-(2,6-dimethylpiperidin-1-yl)propan-2-yl)oxy)-8-fluoroquinazolin-4-yl)-3,8-diazabicyclo[3.2.1]octane-8-carboxylate

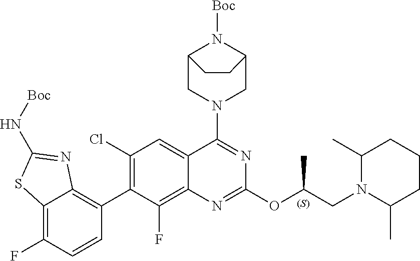

NaH (39.2 mg, 0.98 mmol) was added to a solution of (2S)-1-(2,6-dimethylpiperidin-1-yl) propan-2-ol (84 mg, 0.49 mmol) in THF (15 mL), and the mixture was stirred at 0° C. for 0.5 h. Then a solution of tert-butyl 3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (298 mg, 0.44 mmol) in THF (20 mL) was added and the mixture was stirred at room temperature for 1 h. The resulting mixture was quenched with ice water (10 mL) and extracted with EtOAc (30 mL×3), and the combined organic layer was concentrated to give a residue which purified by chromatography column on silica (eluting with DCM/MeOH=10/1) to give the title product (190 mg, 52%). MS (ESI, m/e) [M+1]⁺828.3.

### Step 2: 4-(4-(3,8-diazabicyclo[3.2.1]octan-3-yl)-6-chloro-2-(((2S)-1-(2,6-dimethylpiperidin-1-yl)propan-2-yl)oxy)-8-fluoroquinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-6-chloro-2-(((2S)-1-(2,6-dimethylpiperidin-1-yl)propan-2-yl)oxy)-8-fluoroquinazolin-4-yl)-3,8-diazabicyclo[3.2.1]octane-8-carboxylate (50 mg, 0.06 mmol) in DCM (15 mL) was added TFA (4 mL), the mixture was stirred at room temperature for 1 h. The resulting mixture was concentrated at room temperature and adjusted pH to 7 with Na₂CO₃, and the organic layer was concentrated to give a residue which was further purified by Prep-HPLC to give title product (25.5 mg, 67%). ¹H NMR (500 MHz, CD₃OD) δ 7.94 (d, J=5.1 Hz, 1H), 7.28-7.17 (m, 1H), 7.00 (m, 1H), 5.50 (m, 1H), 4.60 (m, 2H), 4.02 (m, 2H), 3.90-3.72 (m, 2H), 3.65-3.35 (m, 4H), 2.08-1.95 (m, 4H), 1.79 (m, 3H), 1.68-1.55 (m, 3H), 1.52 (m, 3H), 1.43 (m, 6H). MS (ESI, m/e) [M+1]⁺628.4.

### Example 39: 4-(4-(3,8-diazabicyclo[3.2.1]octan-8-yl)-6-chloro-8-fluoro-2-((octahydroindolizin-2-yl)oxy)quinazolin-7-yl)-7-fluorobenzo[d]thiazol-2-amine

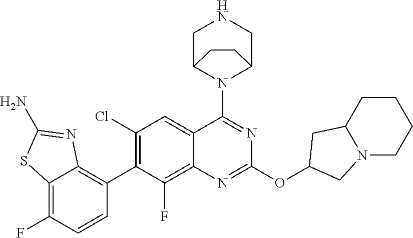 [SMILES: Nc1nc2c(-c3c(Cl)cc4c(N5C6CCC5CNC6)nc(OC5CC6CCCCN6C5)nc4c3F)ccc(F)c2s1]

### Step 1: tert-butyl 8-(7-(2-((tert-butoxycarbonyl)amino)-7-fluorobenzo[d]thiazol-4-yl)-6-chloro-8-fluoro-2-((octahydroindolizin-2-yl)oxy)quinazolin-4-yl)-3,8-diazabicyclo[3.2.1]octane-3-carboxylate

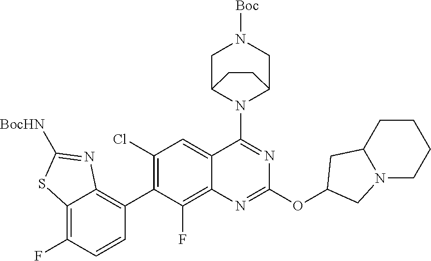 [SMILES: CC(C)(C)OC(=O)Nc1nc2c(-c3c(Cl)cc4c(N5C6CCC5CN(C(=O)OC(C)(C)C)C6)nc(OC5CC6CCCCN6C5)nc4c3F)ccc(F)c2s1]

To a solution of (S)-1-(piperidin-1-yl)propan-2-ol (61 mg, 0.433 mmol) in THF (7.5 mL) was added TBAI (5.5 mg, 0.0148 mmol) and NaH (51 mg, 1.275 mmol). The mixture was stirred at room temperature for about 30 mins. Then, tert-butyl 8-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-3-carboxylate (100 mg, 0.148 mmol) in THF (7.5 mL) was added to the mixture. The mixture was stirred at 40° C. for about 3 hrs and quenched by saturated NH₄Cl aqueous solution, extracted with ethyl acetate. Combined organic layers were washed by brine, dried over anhydrous Na₂SO₄, and evaporated in vacuo. The residue was purified by chromatography (PE/EtOAc=2:1 to 0:1, then DCM/MeOH=1:0 to 4:1) to give the crude product which was used directly in the next step. MS (ESI, m/e) [M+H]⁺ 798.3.

### Step 2: 4-(4-(3,8-diazabicyclo[3.2.1]octan-8-yl)-6-chloro-8-fluoro-2-((octahydroindolizin-2-yl)oxy)quinazolin-7-yl)-7-fluorobenzo[d]thiazol-2-amine

To a solution of tert-butyl 8-(7-(2-((tert-butoxycarbonyl)amino)-7-fluorobenzo[d]thiazol-4-yl)-6-chloro-8-fluoro-2-((octahydroindolizin-2-yl)oxy)quinazolin-4-yl)-3,8-diazabicyclo[3.2.1]octane-3-carboxylate (126 mg, 0.158 mmol) in DCM (20 mL) was added TFA (4 mL). The mixture was stirred at room temperature for overnight. Then, the mixture was evaporated in vacuo. The residue was re-dissolved in MeOH and 7 M NH₃ in MeOH was added to adjust pH >7. Then, the solvent was removed in vacuo and the residue was purified by Prep-HPLC to give the product (25 mg, 27%). ¹H NMR (500 MHz, CD₃OD) δ 7.96 (s, 1H), 7.26-7.17 (m, 1H), 7.00 (t, J=8.8 Hz, 1H), 5.70 (dd, J=12.6, 6.8 Hz, 1H), 5.05 (d, J=6.2 Hz, 2H), 3.79 (d, J=12.9 Hz, 1H), 3.58 (dd, J=24.6, 11.7 Hz, 3H), 3.49 (dd, J=12.3, 6.9 Hz, 1H), 3.37-3.34 (m, 1H), 3.25-3.08 (m, 1H), 2.99-2.87 (m, 2H), 2.66 (s, 1H), 2.26 (dd, J=11.4, 5.4 Hz, 2H), 2.14 (t, J=12.9 Hz, 3H), 1.95 (d, J=13.9 Hz, 3H), 1.78 (d, J=14.8 Hz, 1H), 1.70-1.54 (m, 2H). MS (ESI, m/e) [M+H]⁺ 598.3.

### Example 40: 3-((7-(2-amino-7-fluorobenzo[d]thiazol-4-yl)-4-(3,8-diazabicyclo[3.2.1]octan-3-yl)-6-chloro-8-fluoroquinazolin-2-yl)oxy)-N,N-dimethylpropanamide

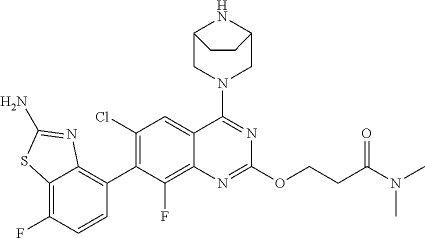 [SMILES: CN(C)C(=O)CCOc1nc(N2CC3CCC(C2)N3)c2cc(Cl)c(-c3ccc(F)c4sc(N)nc34)c(F)c2n1]

### Step 1: tert-butyl 3-(7-(2-((tert-butoxycarbonyl)amino)-7-fluorobenzo[d]thiazol-4-yl)-6-chloro-2-(3-(dimethylamino)-3-oxopropoxy)-8-fluoroquinazolin-4-yl)-3,8-diazabicyclo[3.2.1]octane-8-carboxylate

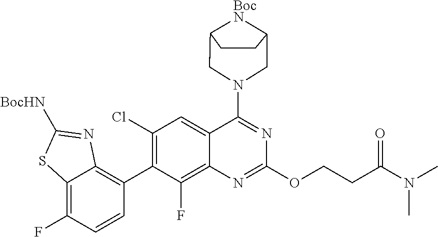 [SMILES: CN(C)C(=O)CCOc1nc(N2CC3CCC(C2)N3C(=O)OC(C)(C)C)c2cc(Cl)c(-c3ccc(F)c4sc(NC(=O)OC(C)(C)C)nc34)c(F)c2n1]

To a solution of 3-hydroxy-N,N-dimethylpropanamide (58 mg, 0.50 mmol) in THF (5 mL) was added Lithium bis(trimethylsilyl)amide (0.8 mL 1.0 M in THF) dropwise at 0° C. The mixture was stirred at 0° C. for 0.5 h. Then tert-butyl 3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (100 mg, 0.15 mmol) in 1 mL tetrahydrofuran was added dropwise and it was stirred at 0° C. for another 0.5 h. The mixture was diluted with dichloromethane and water. The combined organic layer was dried over sodium sulfate and evaporated. The residue was purified by preparative thin layer chromatography to give the title product (30 mg, 26%). MS (ESI, m/e) [M+1]⁺774.4.

### Step 2: 3-((7-(2-amino-7-fluorobenzo[d]thiazol-4-yl)-4-(3,8-diazabicyclo[3.2.1]octan-3-yl)-6-chloro-8-fluoroquinazolin-2-yl)oxy)-N,N-dimethylpropanamide

To a solution of tert-butyl 3-(7-(2-((tert-butoxycarbonyl)amino)-7-fluorobenzo[d]thiazol-4-yl)-6-chloro-2-(3-(dimethylamino)-3-oxopropoxy)-8-fluoroquinazolin-4-yl)-3,8-diazabicyclo[3.2.1]octane-8-carboxylate (30 mg, 0.039 mmol) in 1.5 mL dichloromethane was added 1.0 mL trifluoroacetic acid. The mixture was stirred at room temperature for 4 hrs. Then, solvent was evaporated and the residue was adjusted to pH=11 with aq. Potassium carbonate. The mixture was diluted with dichloromethane:methanol=10:1 solvent and water. Combined organic layer was dried over sodium sulfate and evaporated. The residue was purified by preparative thin layer chromatography to give the product (10 mg, 45%). ¹H NMR (500 MHz, CD₃OD) δ 7.87-7.86 (m, 1H), 7.23-7.17 (m, 1H), 7.01-6.94 (m, 1H), 4.72-4.66 (m, 2H), 4.56-4.39 (m, 2H), 3.71-3.56 (m, 4H), 3.08 (s, 3H), 2.95 (s, 3H), 2.93-2.89 (m, 2H), 1.89-1.75 (m, 4H). MS (ESI, m/e) [M+H]⁺ 574.2.

### Example 41: 4-(4-(3,8-diazabicyclo[3.2.1]octan-8-yl)-6-chloro-8-fluoro-2-(((S)-1-morpholinopropan-2-yl)oxy)quinazolin-7-yl)-7-fluorobenzo[d]thiazol-2-amine

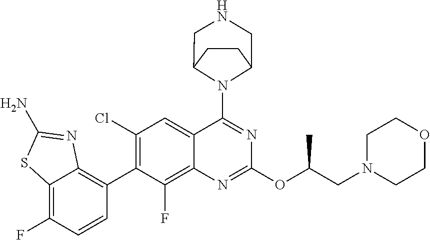 [SMILES: C[C@@H](CN1CCOCC1)Oc1nc(N2C3CCC2CNC3)c2cc(Cl)c(-c3ccc(F)c4sc(N)nc34)c(F)c2n1]

### Step 1: tert-butyl 8-(7-(2-((tert-butoxycarbonyl)amino)-7-fluorobenzo[d]thiazol-4-yl)-6-chloro-8-fluoro-2-(((S)-1-morpholinopropan-2-yl)oxy)quinazolin-4-yl)-3,8-diazabicyclo[3.2.1]octane-3-carboxylate

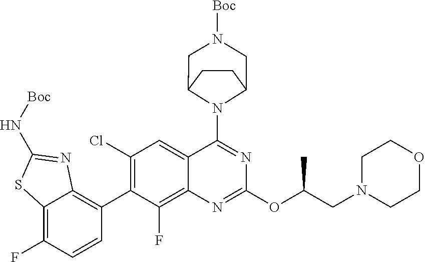 [SMILES: C[C@@H](CN1CCOCC1)Oc1nc(N2C3CCC2CN(C(=O)OC(C)(C)C)C3)c2cc(Cl)c(-c3ccc(F)c4sc(NC(=O)OC(C)(C)C)nc34)c(F)c2n1]

To a solution of (S)-1-morpholinopropan-2-ol (21 mg, 0.148 mmol) in THF (3 mL) was added NaH (9 mg, 0.222 mmol). The mixture was stirred at room temperature for 0.5 h, then a solution of tert-butyl 8-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-3-carboxylate (100 mg, 0.148 mmol) in THF (0.5 mL) was added and the mixture was stirred for another 4 hrs at room temperature. The reaction mixture was diluted with EtOAc and water, extracted with EtOAc twice. Combined organic layer was dried over sodium sulfate and evaporated. The residue was purified by chromatography column on silica (eluting with MeOH/DCM=5-7%) to give the title product (50 mg, 42%). MS (ESI, m/e) [M+1]⁺802.

### Step 2: 4-(4-(3,8-diazabicyclo[3.2.1]octan-8-yl)-6-chloro-8-fluoro-2-(((S)-1-morpholinopropan-2-yl)oxy)quinazolin-7-yl)-7-fluorobenzo[d]thiazol-2-amine

To a solution of tert-butyl 8-(7-(2-((tert-butoxycarbonyl)amino)-7-fluorobenzo[d]thiazol-4-yl)-6-chloro-8-fluoro-2-(((S)-1-morpholinopropan-2-yl)oxy)quinazolin-4-yl)-3,8-diazabicyclo[3.2.1]octane-3-carboxylate (50 mg, 0.062 mmol) in EtOAc (20 mL) was added HCl/dioxane (2 mL, 4 M) and stirred for 4 hrs at room temperature. Then the resulting mixture was filtered to give the crude product, which was purified by prep-HPLC to give the title product (2.4 mg, 6%). ¹H NMR (500 MHz, CD₃OD) δ 7.93 (s, 1H), 7.24-7.17 (m, 1H), 6.99 (t, J=8.8 Hz, 1H), 5.64-5.52 (m, 1H), 5.02 (s, 2H), 3.69-3.48 (m, 6H), 3.27 (s, 2H), 2.92-2.80 (m, 1H), 2.76-2.57 (m, 5H), 2.23 (dd, J=14.7, 9.5 Hz, 2H), 2.11 (d, J=8.2 Hz, 2H), 1.40 (dd, J=6.2, 1.3 Hz, 3H). MS (ESI, m/e) [M+H]⁺ 602.5.

### Example 42: 8-(4-(3,8-diazabicyclo[3.2.1]octan-3-yl)-6-chloro-8-fluoro-2-(((2S,4R)-4-fluoro-1-methylpyrrolidin-2-yl)methoxy)quinazolin-7-yl)quinolin-2-amine

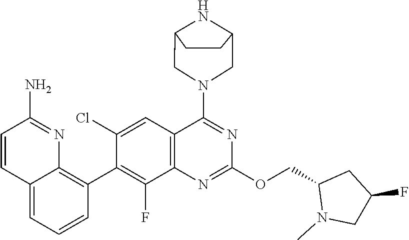 [SMILES: CN1C[C@H](F)C[C@H]1COc1nc(N2CC3CCC(C2)N3)c2cc(Cl)c(-c3cccc4ccc(N)nc34)c(F)c2n1]

### Step 1: tert-butyl 3-(7-(2-aminoquinolin-8-yl)-6-chloro-2,8-difluoroquinazolin-4-yl)-3,8-diazabicyclo[3.2.1]octane-8-carboxylate

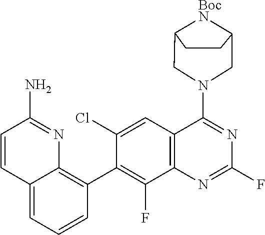 [SMILES: CC(C)(C)OC(=O)N1C2CCC1CN(c1nc(F)nc3c(F)c(-c4cccc5ccc(N)nc45)c(Cl)cc13)C2]

A mixture of 2-aminoquinolin-8-yl boronic acid (207 mg), dioxane (30 mL), H₂O (3 mL), tert-butyl 3-(7-bromo-6-chloro-2,8-difluoroquinazolin-4-yl)-3,8-diazabicyclo[3.2.1]octane-8-carboxylate (240 mg), Pd(dppf)Cl₂ (46.6 mg), and Na₂CO₃ (135 mg) was stirred for 2 hrs at 100° C. The resulting mixture was concentrated under vacuum and the residue was applied onto a silica gel column with ethyl acetate/petroleum ether (1:1) to give the title product (70 mg, 26%). MS (ESI, m/e) [M+H]⁺ 553.

### Step 2: tert-butyl 3-(7-(2-aminoquinolin-8-yl)-6-chloro-8-fluoro-2-(((2S,4R)-4-fluoro-1-methylpyrrolidin-2-yl)methoxy)quinazolin-4-yl)-diazabicyclo[3.2.1]octane-8-carboxylate

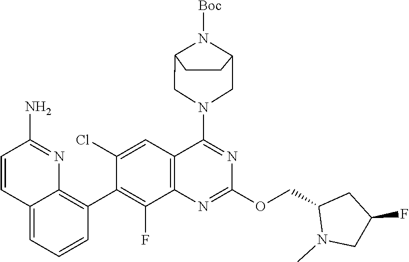 [SMILES: CN1C[C@H](F)C[C@H]1COc1nc(N2CC3CCC(C2)N3C(=O)OC(C)(C)C)c2cc(Cl)c(-c3cccc4ccc(N)nc34)c(F)c2n1]

A mixture of [(2S,4R)-4-fluoro-1-methylpyrrolidin-2-yl]methanol (23.5 mg, 0.18 mmol), DMF (5 mL), and NaH (6 mg, 60% wt) was stirred for 30 min at room temperature. Then, tert-butyl 3-(7-(2-aminoquinolin-8-yl)-6-chloro-2,8-difluoroquinazolin-4-yl)-3,8-diazabicyclo[3.2.1]octane-8-carboxylate (75 mg, 0.14 mmol) was added and the mixture was stirred for 2 hrs at room temperature. Solvents were removed and the residue was applied onto a silica gel column with dichloromethane/methanol (1:10) to give the title product (60 mg, 66%). MS (ESI, m/e) [M+H]⁺ 666.2.

### Step 3: 8-(4-(3,8-diazabicyclo[3.2.1]octan-3-yl)-6-chloro-8-fluoro-2-(((2S,4R)-4-fluoro-1-methylpyrrolidin-2-yl)methoxy)quinazolin-7-yl)quinolin-2-amine

To a solution of tert-butyl 3-(7-(2-aminoquinolin-8-yl)-6-chloro-8-fluoro-2-(((2S,4R)-4-fluoro-1-methylpyrrolidin-2-yl)methoxy)quinazolin-4-yl)-3,8-diazabicyclo[3.2.1]octane-8-carboxylate (60 mg, 0.09 mmol) in DCM (4 mL) was added TFA (2 mL). The resulting solution was stirred for 1 h at room temperature and concentrated under vacuum. Crude product was purified by Prep-HPLC to give the title product (21 mg, 34%). ¹H NMR (400 MHz, CD₃OD) δ 8.47 (dd, J=9.6, 2.4 Hz, 1H), 8.15-7.98 (m, 2H), 7.78-7.66 (m, 2H), 7.16 (m, 1H), 5.47 (m, 1H), 5.15-4.94 (m, 1H), 4.83-4.61 (m, 3H), 4.28 (d, J=4.8 Hz, 3H), 4.10 (t, J=18.4 Hz, 1H), 3.99-3.80 (m, 2H), 3.64 (q, J=17.6 Hz, 1H), 3.21 (d, J=6.8 Hz, 3H), 2.77-2.56 (m, 1H), 2.56-2.32 (m, 1H), 2.19 (m, 4H). MS (ESI, m/e) [M+H]⁺ 566.2.

### Example 43: 4-(4-(3,8-diazabicyclo[3.2.1]octan-8-yl)-6-chloro-8-fluoro-2-(((2S)-1-(3-fluoropyrrolidin-1-yl)propan-2-yl)oxy)quinazolin-7-yl)-7-fluorobenzo[d]thiazol-2-amine

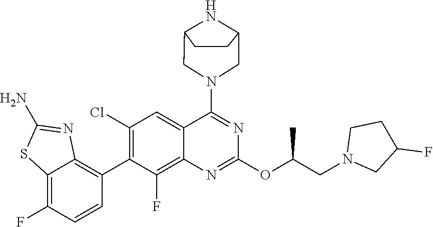 [SMILES: C[C@@H](CN1CCC(F)C1)Oc1nc(N2CC3CCC(C2)N3)c2cc(Cl)c(-c3ccc(F)c4sc(N)nc34)c(F)c2n1]

### Step 1: tert-butyl 8-(7-(2-((tert-butoxycarbonyl)amino)-7-fluorobenzo[d]thiazol-4-yl)-6-chloro-8-fluoro-2-(((2S)-1-(3-fluoropyrrolidin-1-yl)propan-2-yl)oxy)quinazolin-4-yl)-3,8-diazabicycl[3.2.1]octane-3-carboxylate

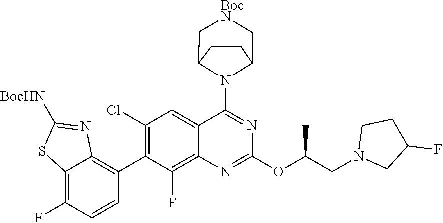 [SMILES: C[C@@H](CN1CCC(F)C1)Oc1nc(N2C3CCC2CN(C(=O)OC(C)(C)C)C3)c2cc(Cl)c(-c3ccc(F)c4sc(NC(=O)OC(C)(C)C)nc34)c(F)c2n1]

To a solution of (2S)-1-(3-fluoropyrrolidin-1-yl)propan-2-ol (100 mg, 0.70 mmol) in 3 mL tetrahydrofuran was added sodium hydride (60%) (36 mg, 0.9 mmol) at 0° C. The mixture was stirred at room temperature for 1 h. Then, tert-butyl (8-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-3-carboxylate (100 mg, 0.15 mmol) in 1 mL tetrahydrofuran was added dropwise. The mixture was stirred at room temperature for another 1 h and diluted with dichloromethane and water. Combined organic layer was dried over sodium sulfate and evaporated. The residue was purified by preparative thin layer chromatography to give the title product (20 mg, 16.8%). MS (ESI, m/e) [M+1]⁺804.4.

### Step 2: 4-(4-(3,8-diazabicyclo[3.2.1]octan-8-yl)-6-chloro-8-fluoro-2-(((2S)-1-(3-fluoropyrrolidin-1-yl)propan-2-yl)oxy)quinazolin-7-yl)-7-fluorobenzo[d]thiazol-2-amine

To a solution of tert-butyl 8-(7-(2-((tert-butoxycarbonyl)amino)-7-fluorobenzo[d]thiazol-4-yl)-6-chloro-8-fluoro-2-(((2S)-1-(3-fluoropyrrolidin-1-yl)propan-2-yl)oxy)quinazolin-4-yl)-3,8-diazabicyclo[3.2.1]octane-3-carboxylate (20 mg, 0.024 mmol) in dichloromethane (1.5 mL) was added 1.0 mL trifluoroacetic acid and the mixture was stirred at room temperature for 3 hrs. Solvent was evaporated and the residue was adjusted to pH=11 with aq. Potassium carbonate and then diluted with dichloromethane:methanol=10:1 solvent and water. Combined organic layer was dried over sodium sulfate and evaporated. The residue was lyophilized to give the title product (7 mg, 47%). ¹H NMR (400 MHz, CD₃OD) δ 7.90 (s, 1H), 7.25-7.18 (m, 1H), 7.01-6.94 (m, 1H), 5.55-5.43 (m, 1H), 5.22-5.03 (m, 1H), 4.96-4.87 (m, 2H), 3.25-3.16 (m, 2H), 3.08-2.73 (m, 7H), 2.66-2.45 (m, 2H), 2.20-1.90 (m, 7H), 1.37-1.36 (d, J=1.4 Hz, 3H). MS (ESI, m/e) [M+H]⁺ 604.4.

### Example 44: (2S,4R)-1-((2S)-2-(2-((1-(2-((7-(2-amino-7-fluorobenzo[d]thiazol-4-yl)-4-(3,8-diazabicyclo[3.2.1]octan-3-yl)-6-chloro-8-fluoroquinazolin-2-yl)oxy)propyl)piperidin-4-yl)oxy)acetamido)-3,3-dimethylbutanoyl)-4-hydroxy-N—((S)-1-(4-(4-methylthiazol-5-yl)phenyl)ethyl)pyrrolidine-2-carboxamide

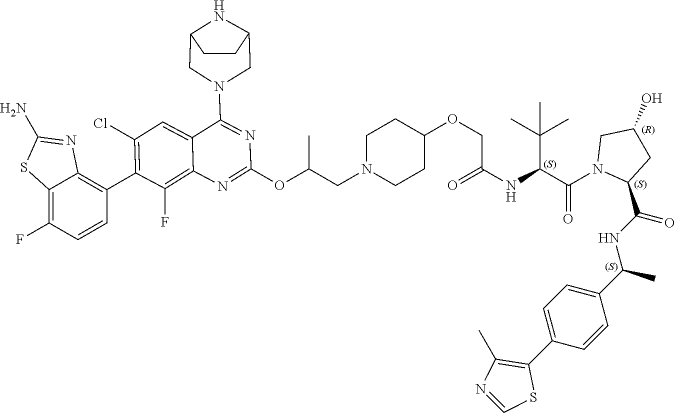

Synthetic Route:

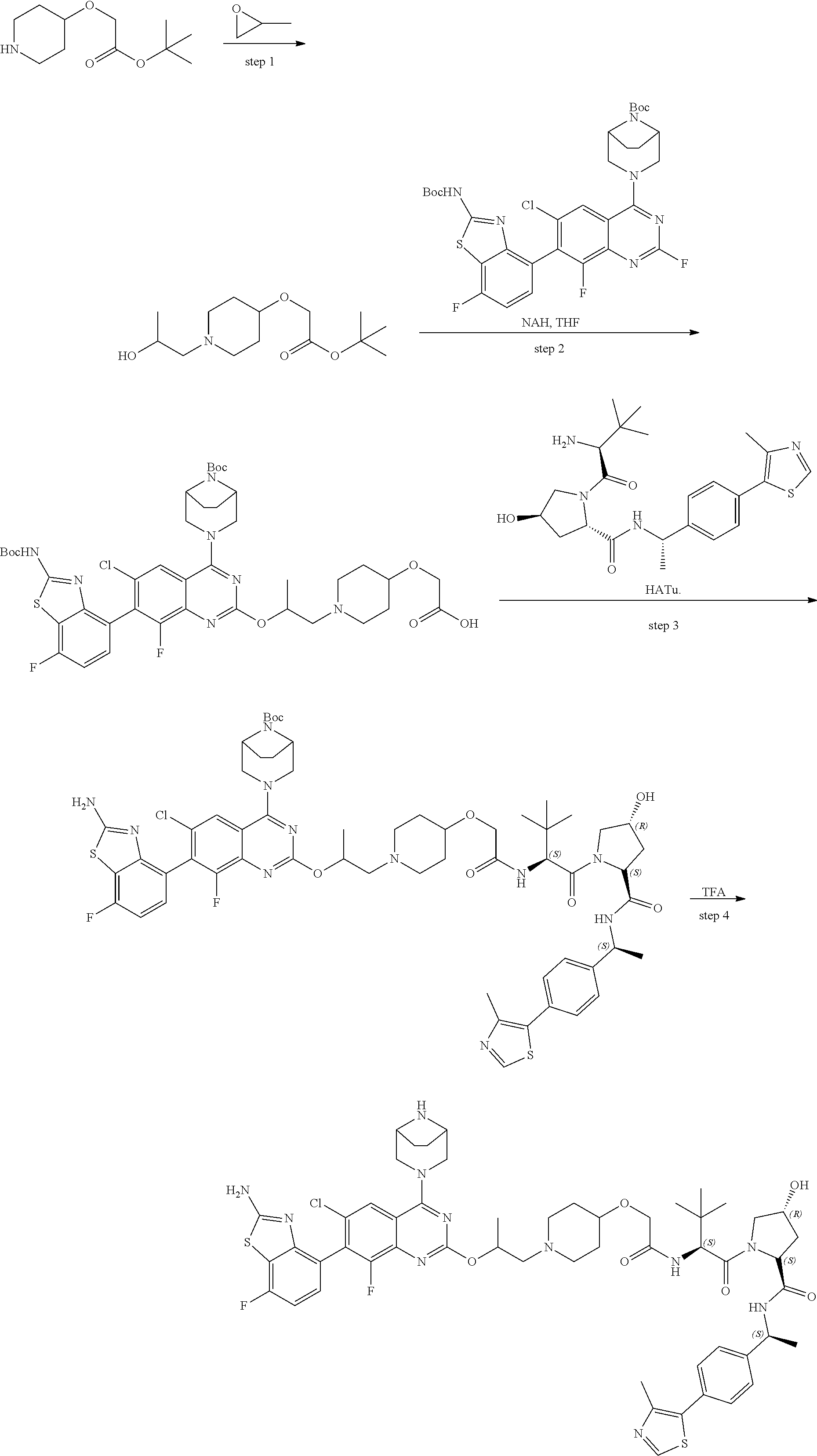

### Step 1: tert-butyl 2-((1-(2-hydroxypropyl)piperidin-4-yl)oxy)acetate

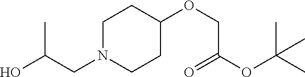 [SMILES: CC(O)CN1CCC(OCC(=O)OC(C)(C)C)CC1]

To a solution of tert-butyl 2-(piperidin-4-yloxy)acetate (500 mg, 2.31 mmol) in 4 mL water and 1 mL tetrahydrofuran was added 2-methyloxirane (120 mg, 2.07 mmol) at 0° C. The mixture was stirred at room temperature for 16 hrs and then diluted with dichloromethane. Combined organic layer was dried over sodium sulfate and evaporated. The residue was purified by chromatography column on silica to give the title compound (300 mg, 53%). MS (ESI, m/e) [M+1]⁺274.2.

### Step 2: 2-((1-(2-((4-(8-(tert-butoxycarbonyl)-3,8-diazabicyclo[3.2.1]octan-3-yl)-7-(2-((tert-butoxycarbonyl)amino)-7-fluorobenzo[d]thiazol-4-yl)-6-chloro-8-fluoroquinazolin-2-yl)oxy)propyl)piperidin-4-yl)oxy)acetic acid

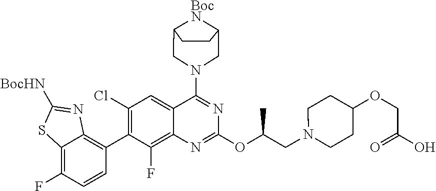 [SMILES: C[C@@H](CN1CCC(OCC(=O)O)CC1)Oc1nc(N2CC3CCC(C2)N3C(=O)OC(C)(C)C)c2cc(Cl)c(-c3ccc(F)c4sc(NC(=O)OC(C)(C)C)nc34)c(F)c2n1]

To a solution of tert-butyl 2-((1-(2-hydroxypropyl)piperidin-4-yl)oxy)acetate (300 mg, 1.10 mmol) in THF (10 mL) was added sodium hydride (60%) (72 mg, 1.8 mmol) at 0° C. The mixture was stirred at room temperature for 1 h. Then, tert-butyl 3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (200 mg, 0.30 mmol) in 1 mL tetrahydrofuran was added dropwise. The mixture was stirred at room temperature for 1 h and diluted with dichloromethane and water. Combined organic layer was dried over sodium sulfate and evaporated. The residue was purified by preparative thin layer chromatography to give the title compound (40 mg, 15%). MS (ESI, m/e) [M+1]⁺874.5.

### Step 3: tert-butyl 3-(7-(2-((tert-butoxycarbonyl)amino)-7-fluorobenzo[d]thiazol-4-yl)-6-chloro-8-fluoro-2-((1-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)piperidin-1-yl)propan-2-yl)oxy)quinazolin-4-yl)-3,8-diazabicyclo[3.2.1]octane-8-carboxylate

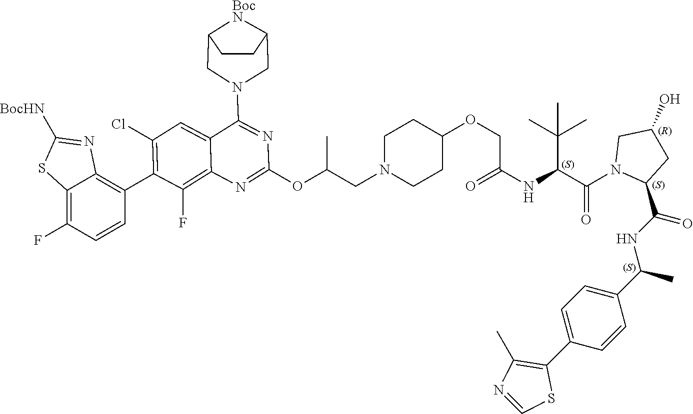

To a solution of 2-((1-(2-((4-(8-(tert-butoxycarbonyl)-3,8-diazabicyclo[3.2.1]octan-3-yl)-7-(2-((tert-butoxycarbonyl)amino)-7-fluorobenzo[d]thiazol-4-yl)-6-chloro-8-fluoroquinazolin-2-yl)oxy)propyl)piperidin-4-yl)oxy)acetic acid (40 mg, 0.046 mmol) in dichloromethane (3 mL) was added HATU (23 mg, 0.06 mmol) and DIPEA (0.1 mL). The mixture was stirred at room temperature for 1 h. Then, (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (24 mg, 0.05 mmol) was added and the mixture was stirred at room temperature for 4 hrs, and it was diluted with dichloromethane and water. Combined organic layer was dried over sodium sulfate and evaporated. The residue was purified by preparative thin layer chromatography to give the title compound (40 mg, 67%). MS (ESI, m/e) [M+1]⁺1300.4.

### Step 4: (2S,4R)-1-((2S)-2-(2-((1-(2-((7-(2-amino-7-fluorobenzo[d]thiazol-4-yl)-4-(3,8-diazabicyclo[3.2.1]octan-3-yl)-6-chloro-8-fluoroquinazolin-2-yl)oxy)propyl)piperidin-4-yl)oxy)acetamido)-3,3-dimethylbutanoyl)-4-hydroxy-N—((S)-1-(4-(4-methylthiazol-5-yl)phenyl)ethyl)pyrrolidine-2-carboxamide

To a solution of tert-butyl 3-(7-(2-((tert-butoxycarbonyl)amino)-7-fluorobenzo[d]thiazol-4-yl)-6-chloro-8-fluoro-2-((1-(4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)piperidin-1-yl)propan-2-yl)oxy)quinazolin-4-yl)-3,8-diazabicyclo[3.2.1]octane-8-carboxylate (40 mg, 0.031 mmol) in dichloromethane (1.5 mL) was added trifluoroacetic acid (1.0 mL) and the mixture was stirred at room temperature for 4 hrs. Solvent was evaporated and the residue was purified by Prep-HPLC to give the title compound (13 mg, 38.4%). ¹H NMR (400 MHz, CD₃OD) δ 8.87 (s, 1H), 7.89 (s, 1H), 7.47-7.33 (m, 4H), 7.24-7.18 (m, 1H), 7.03-6.96 (m, 1H), 5.65-5.57 (m, 1H), 5.04-4.96 (m, 1H), 4.64-4.40 (m, 5H), 4.09-3.92 (m, 4H), 3.85-3.68 (m, 4H), 3.67-3.48 (m, 2H), 3.22-3.04 (m, 2H), 2.97-2.72 (m, 2H), 2.49-2.42 (m, 3H), 2.09-1.89 (m, 8H), 1.86-1.65 (m, 2H), 1.54-1.36 (m, 7H), 1.05-0.97 (m, 9H). MS (ESI, m/e) [M+H]⁺ 1100.5.

### Example 45: (2S,4R)-1-((2S)-2-(7-((2S)-2-(((7-(2-amino-7-fluorobenzo[d]thiazol-4-yl)-4-(3,8-diazabicyclo[3.2.1]octan-8-yl)-6-chloro-8-fluoroquinazolin-2-yl)oxy)methyl)pyrrolidin-1-yl)heptanamido)-3,3-dimethylbutanoyl)-4-hydroxy-N—((S)-1-(4-(4-methylthiazol-5-yl)phenyl)ethyl)pyrrolidine-2-carboxamide

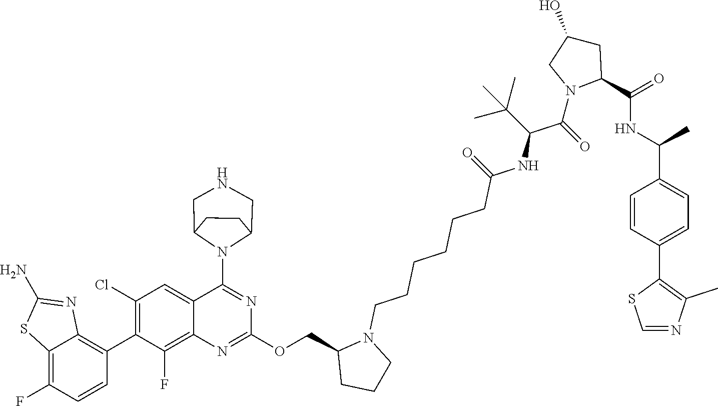 [SMILES: Cc1ncsc1-c1ccc([C@H](C)NC(=O)[C@@H]2C[C@@H](O)CN2C(=O)[C@@H](NC(=O)CCCCCCN2CCC[C@H]2COc2nc(N3C4CCC3CNC4)c3cc(Cl)c(-c4ccc(F)c5sc(N)nc45)c(F)c3n2)C(C)(C)C)cc1]

Synthetic Route

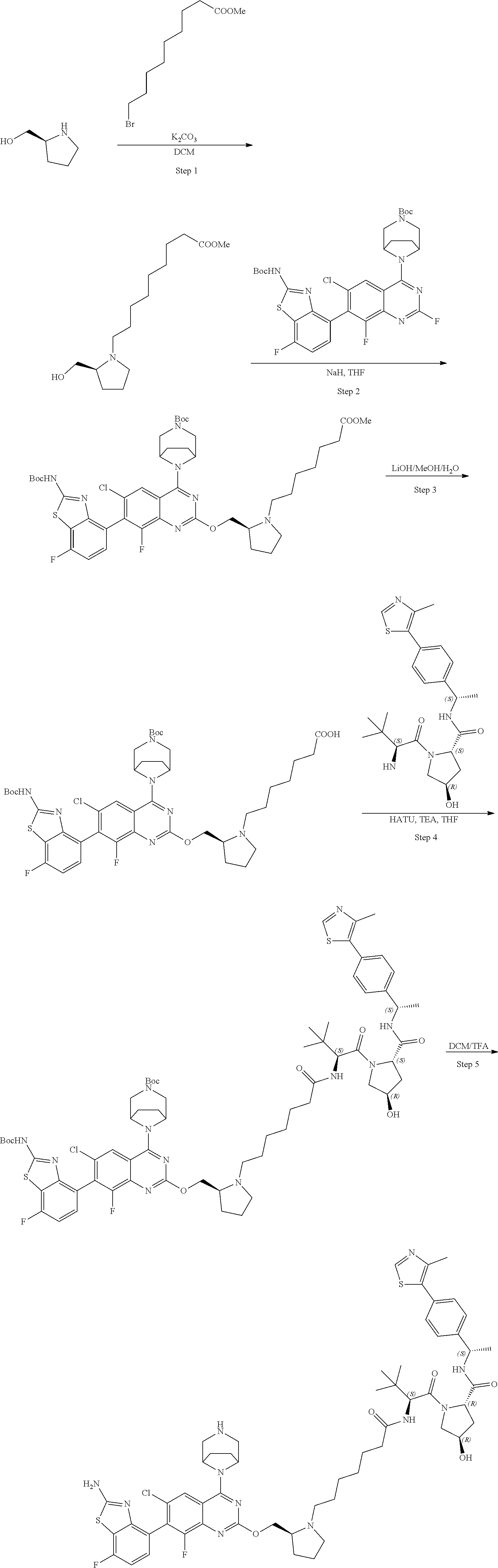

### Step 1: methyl (S)-7-(2-(hydroxymethyl)pyrrolidin-1-yl)heptanoate

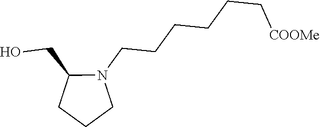 [SMILES: COC(=O)CCCCCCN1CCC[C@H]1CO]

To a mixture of (S)-pyrrolidin-2-ylmethanol (1.01 g, 10 mmol) and K₂CO₃ (2.76 g, 20 mmol) in DMF (20 ml) was added methyl 7-bromoheptanoate (2.51 g, 10 mmol). The resulting mixture was stirred for 16 hrs at room temperature. After completion, the reaction mixture was diluted with EtOAc (100 mL), and washed with saturated NaCl (30 mL×3). Combined organic layer was dried over anhydrous Na₂SO₄, filtered and the filtrate was concentrated to give the crude product which was used directly in next step. MS (ESI, m/e) [M+1]⁺244.2.

### Step 2: tert-butyl 8-(7-(2-((tert-butoxycarbonyl)amino)-7-fluorobenzo[d]thiazol-4-yl)-6-chloro-8-fluoro-2-(((S)-1-(7-methoxy-7-oxoheptyl)pyrrolidin-2-yl)methoxy)quinazolin-4-yl)-3,8-diazabicyclo[3.2.1]octane-3-carboxylate

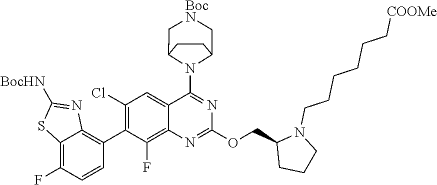 [SMILES: COC(=O)CCCCCCN1CCC[C@H]1COc1nc(N2C3CCC2CN(C(=O)OC(C)(C)C)C3)c2cc(Cl)c(-c3ccc(F)c4sc(NC(=O)OC(C)(C)C)nc34)c(F)c2n1]

To a solution of methyl (S)-7-(2-(hydroxymethyl)pyrrolidin-1-yl)heptanoate (1 g, 4.09 mmol) in THF (20 mL) was added sodium hydride (163 mg, 4.09 mmol) at 0° C. The resulting mixture was stirred at 0° C. for 1 h. Then, tert-butyl 8-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-3-carboxylate (923 mg, 1.36 mmol) was added and the reaction mixture was stirred for 16 hrs. The reaction mixture was quenched with water (20 ml), and extracted with DCM (50 ml×3). Combined organic layer was dried over anhydrous Na₂SO₄, filtered and the filtrate was concentrated under reduced pressure to give crude product, which was then purified by silica gel column chromatography, eluted with 0-10% MeOH in DCM to give the title product (200 mg, 16%). MS (ESI, m/e) [M+1]⁺900.4.

### Step 3: 7-((2S)-2-(((4-(3-(tert-butoxycarbonyl)-3,8-diazabicyclo[3.2.1]octan-8-yl)-7-(2-((tert-butoxycarbonyl)amino)-7-fluorobenzo[d]thiazol-4-yl)-6-chloro-8-fluoroquinazolin-2-yl)oxy)methyl)pyrrolidin-1-yl)heptanoic acid

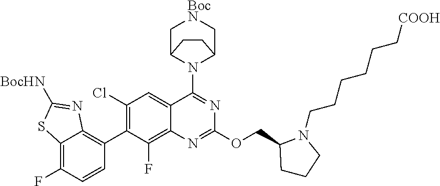 [SMILES: CC(C)(C)OC(=O)Nc1nc2c(-c3c(Cl)cc4c(N5C6CCC5CN(C(=O)OC(C)(C)C)C6)nc(OC[C@@H]5CCCN5CCCCCCC(=O)O)nc4c3F)ccc(F)c2s1]

To a mixture of tert-butyl 8-(7-(2-((tert-butoxycarbonyl)amino)-7-fluorobenzo[d]thiazol-4-yl)-6-chloro-8-fluoro-2-(((S)-1-(7-methoxy-7-oxoheptyl)pyrrolidin-2-yl)methoxy)quinazolin-4-yl)-3,8-diazabicyclo[3.2.1]octane-3-carboxylate (200 mg, 0.222 mmol) in MeOH (3 mL) and THF (3 mL) was added 1 M LiOH solution (3 mL, 3 mmol). The resulting mixture was stirred for 16 hrs at room temperature. Upon completion, the reaction mixture was neutralized with 1 M HCl, and then extracted with DCM (40 mL×3). The organic layer was dried over anhydrous Na₂SO₄, filtered and the filtrate was concentrated to give the title product (150 mg, 76%). MS (ESI, m/e) [M+1]⁺886.4.

### Step 4: tert-butyl 8-(7-(2-((tert-butoxycarbonyl)amino)-7-fluorobenzo[d]thiazol-4-yl)-6-chloro-8-fluoro-2-(((S)-1-(7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptyl)pyrrolidin-2-yl)methoxy)quinazolin-4-yl)-3,8-diazabicyclo [3.2.1]octane-3-carboxylate

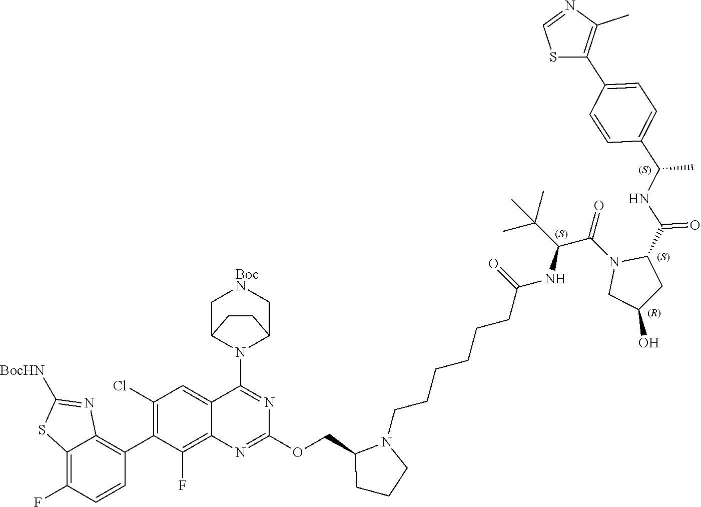

To a mixture of 7-((2S)-2-(((4-(3-(tert-butoxycarbonyl)-3,8-diazabicyclo[3.2.1]octan-8-yl)-7-(2-((tert-butoxycarbonyl)amino)-7-fluorobenzo[d]thiazol-4-yl)-6-chloro-8-fluoroquinazolin-2-yl)oxy)methyl)pyrrolidin-1-yl)heptanoic acid (70 mg, 0.079 mmol), (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride (76.06 mg, 0.158 mmol) and TEA (0.1 mL) in THF (10 mL) was added HATU (60.08 mg, 0.158 mmol). The resulting mixture was stirred at room temperature for 15 hrs. Upon completion, the reaction mixture was purified by Prep-HPLC to give the title product (60 mg, 58%). MS (ESI, m/e) [M+H]⁺ 1312.56.

### Step 5: (2S,4R)-1-((2S)-2-(7-((2S)-2-(((7-(2-amino-7-fluorobenzo[d]thiazol-4-yl)-4-(3,8-diazabicyclo[3.2.1]octan-8-yl)-6-chloro-8-fluoroquinazolin-2-yl)oxy)methyl)pyrrolidin-1-yl)heptanamido)-3,3-dimethylbutanoyl)-4-hydroxy-N—((S)-1-(4-(4-methylthiazol-5-yl)phenyl)ethyl)pyrrolidine-2-carboxamide

To a solution of tert-butyl 8-(7-(2-((tert-butoxycarbonyl)amino)-7-fluorobenzo[d]thiazol-4-yl)-6-chloro-8-fluoro-2-(((S)-1-(7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptyl)pyrrolidin-2-yl)methoxy)quinazolin-4-yl)-3,8-diazabicyclo[3.2.1]octane-3-carboxylate

(30 mg, 0.023 mmol) in DCM (10 mL) was added TFA (2 mL). The resulting solution was stirred for 16 hrs at room temperature. Upon completion, the reaction mixture was concentrated to give a residue which was further purified by Prep-HPLC to give the title product (4.3 mg, 17%). ¹H NMR (500 MHz, DMSO-d₆) δ 8.98 (s, 1H), 8.36 (d, J=10.0 Hz, 1H), 7.92 (s, 2H), 7.86 (s, 1H), 7.76 (d, J=10 Hz, 1H), 7.44-7.37 (m, 4H), 7.24-7.22 (m, 1H), 7.07-7.04 (m, 1H), 4.93-4.90 (m, 1H), 4.80-4.78 (m, 2H), 4.51-4.49 (m, 1H), 4.43-4.40 (m, 1H), 4.35-4.32 (m, 1H), 4.28-4.26 (m, 1H), 4.11-4.03 (m, 1H), 3.62-3.57 (m, 2H), 3.08-3.03 (m, 3H), 2.83-2.77 (m, 4H), 2.45 (s, 3H), 2.33-2.16 (m, 3H), 2.11-1.86 (m, 7H), 1.82-1.76 (m, 1H), 1.74-1.62 (m, 3H), 1.53-1.36 (m, 7H), 1.23-1.17 (m, 4H), 0.91 (s, 9H). MS (ESI, m/e) [M+H]⁺ 1112.8.

### Example 46: (2S,4R)-1-((2S)-2-(7-((2S)-2-(((7-(2-amino-7-fluorobenzo[d]thiazol-4-yl)-4-(3,8-diazabicyclo[3.2.1]octan-3-yl)-6-chloro-8-fluoroquinazolin-2-yl)oxy)methyl)pyrrolidin-1-yl)heptanamido)-3,3-dimethylbutanoyl)-4-hydroxy-N—((S)-1-(4-(4-methylthiazol-5-yl)phenyl)ethyl)pyrrolidine-2-carboxamide

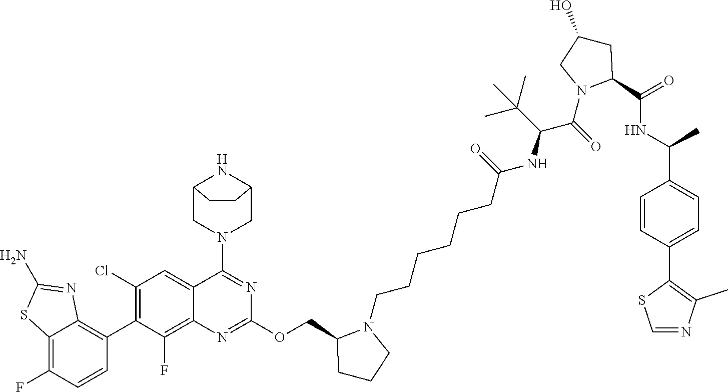 [SMILES: Cc1ncsc1-c1ccc([C@H](C)NC(=O)[C@@H]2C[C@@H](O)CN2C(=O)[C@@H](NC(=O)CCCCCCN2CCC[C@H]2COc2nc(N3CC4CCC(C3)N4)c3cc(Cl)c(-c4ccc(F)c5sc(N)nc45)c(F)c3n2)C(C)(C)C)cc1]

Example 46 was prepared by similar procedure as described in Example 44 from tert-butyl 3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate, methyl (S)-7-(2-(hydroxymethyl)pyrrolidin-1-yl)heptanoate and (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide. ¹H NMR (500 MHz, DMSO-d₆) δ 8.99 (s, 1H), 8.36 (d, J=10.0 Hz, 1H), 7.91-7.89 (m, 3H), 7.77 (d, J=10.0 Hz, 1H), 7.44-7.37 (m, 4H), 7.22-7.19 (m, 1H), 7.09-7.05 (m, 1H), 5.11-4.09 (m, 1H), 4.93-4.88 (m, 1H), 4.51-4.39 (m, 5H), 4.28 (s, 1H), 4.12 (s, 2H), 3.74-3.71 (m, 2H), 3.60-3.52 (m, 2H), 3.30-2.82 (m, 4H), 2.45 (s, 3H), 2.28-2.21 (m, 1H), 2.14-1.97 (m, 3H), 1.95-1.71 (m, 7H), 1.55-1.45 (m, 6H), 1.37 (d, J=10 Hz, 3H), 1.28-1.22 (m, 4H), 0.91 (s, 9H). MS (ESI, m/e) [M+H]⁺ 1112.8.

### Example 47: (2S,4R)-1-((2S)-2-(4-(1-((2S)-2-((7-(2-amino-7-fluorobenzo[d]thiazol-4-yl)-4-(3,8-diazabicyclo[3.2.1]octan-3-yl)-6-chloro-8-fluoroquinazolin-2-yl)oxy)propyl)piperidin-4-yl)butanamido)-3,3-dimethylbutanoyl)-4-hydroxy-N—((S)-1-(4-(4-methylthiazol-5-yl)phenyl)ethyl)pyrrolidine-2-carboxamide

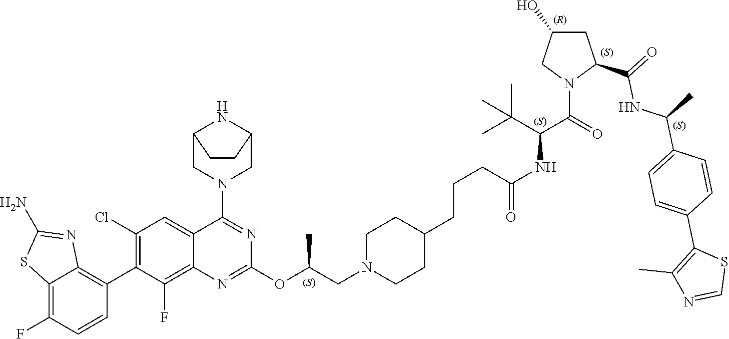

### Step 1: tert-butyl 3-(7-(2-((tert-butoxycarbonyl)amino)-7-fluorobenzo[d]thiazol-4-yl)-6-chloro-8-fluoro-2-(((S)-1-(4-(4-methoxy-4-oxobutyl)piperidin-1-yl)propan-2-yl)oxy)quinazolin-4-yl)-3,8-diazabicyclo[3.2.1]octane-8-carboxylate

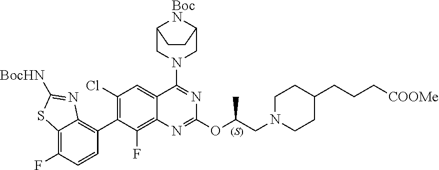

NaH (67 mg, 1.675 mmol) was added to a solution of methyl (S)-4-(1-(2-hydroxypropyl)piperidin-4-yl)butanoate (240 mg, 1 mmol) in THF (20 mL), and the mixture was stirred at 0° C. for 0.5 h. Then, a solution of tert-butyl 3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (450 mg, 0.67 mmol) in THF (10 mL) was added, and the mixture was stirred at room temperature for 1 h. The resulting mixture was quenched with ice water (15 mL) and extracted with EtOAc (40 mL×3). Combined organic layer was concentrated and the crude product was purified by chromatography column on silica (eluting with DCM/MeOH=50/1) to give the title product (478 mg, 80%). MS (ESI, m/e) [M+1]⁺900.3.

### Step 2: 4-(1-((2S)-2-((4-(8-(tert-butoxycarbonyl)-3,8-diazabicyclo[3.2.1]octan-3-yl)-7-(2-((tert-butoxycarbonyl)amino)-7-fluorobenzo[d]thiazol-4-yl)-6-chloro-8-fluoroquinazolin-2-yl)oxy)propyl)piperidin-4-yl)butanoic acid

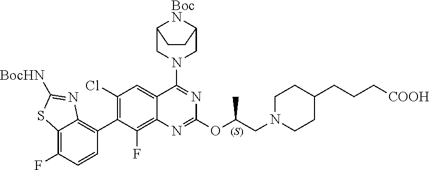

To a solution of tert-butyl 3-(7-(2-((tert-butoxycarbonyl)amino)-7-fluorobenzo[d]thiazol-4-yl)-6-chloro-8-fluoro-2-(((S)-1-(4-(4-methoxy-4-oxobutyl)piperidin-1-yl)propan-2-yl)oxy)quinazolin-4-yl)-3,8-diazabicyclo[3.2.1]octane-8-carboxylate (180 mg, 0.2 mmol) in MeOH/THF/H₂O (6 mL/6 mL/2 mL) was added 3 mL LiOH (1M in water), and the mixture was stirred at room temperature for 1 h. The resulting mixture was concentrated and pH was adjusted to 7 with 1 N HCl. Then diluted with water and extracted with DCM (20 mL×3). Combined organic layer was concentrated to give the crude product which was used directly in the next step. MS (ESI, m/e) [M+1]⁺886.3.

### Step 3: tert-butyl 3-(7-(2-((tert-butoxycarbonyl)amino)-7-fluorobenzo[d]thiazol-4-yl)-6-chloro-8-fluoro-2-(((S)-1-(4-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)piperidin-1-yl)propan-2-yl)oxy)quinazolin-4-yl)-3,8-diazabicyclo[3.2.1]octane-8-carboxylate

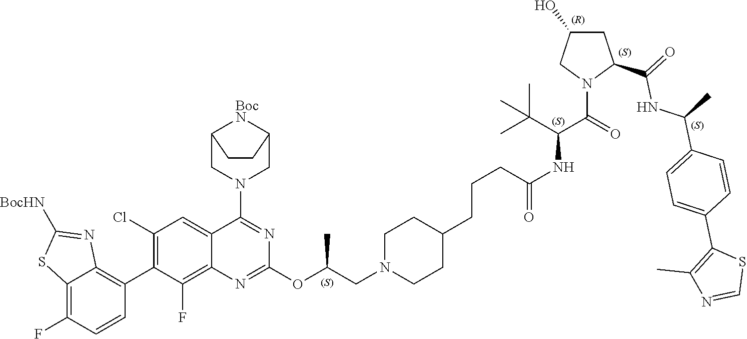

To a solution of 4-(1-((2S)-2-((4-(8-(tert-butoxycarbonyl)-3,8-diazabicyclo[3.2.1]octan-3-yl)-7-(2-((tert-butoxycarbonyl)amino)-7-fluorobenzo[d]thiazol-4-yl)-6-chloro-8-fluoroquinazolin-2-yl)oxy)propyl)piperidin-4-yl)butanoic acid (65 mg, 0.073 mmol) in THF (15 mL) was added (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (53 mg, 0.11 mmol), HATU (42 mg, 0.11 mmol) and DIPEA (23.5 mg, 0.18 mmol), and the mixture was stirred at room temperature for 0.5 h. The resulting mixture was concentrated and the crude product was purified by chromatography column on silica (eluting with DCM/MeOH=10/1) to give the title product (81 mg, 84%). MS (ESI, m/e) [M+1]⁺1312.5.

### Step 4: (2S,4R)-1-((2S)-2-(4-(1-((2S)-2-((7-(2-amino-7-fluorobenzo[d]thiazol-4-yl)-4-(3,8-diazabicyclo[3.2.1]octan-3-yl)-6-chloro-8-fluoroquinazolin-2-yl)oxy)propyl)piperidin-4-yl)butanamido)-3,3-dimethylbutanoyl)-4-hydroxy-N—((S)-1-(4-(4-methylthiazol-5-yl)phenyl)ethyl)pyrrolidine-2-carboxamide

To a solution of tert-butyl 3-(7-(2-((tert-butoxycarbonyl)amino)-7-fluorobenzo[d]thiazol-4-yl)-6-chloro-8-fluoro-2-(((S)-1-(4-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutyl)piperidin-1-yl)propan-2-yl)oxy)quinazolin-4-yl)-3,8-diazabicyclo[3.2.1]octane-8-carboxylate (81 mg, 0.062 mmol) in DCM (10 mL) was added TFA (3 mL) at room temperature, and the mixture was stirred at room temperature for 1 h. The resulting mixture was concentrated and pH was adjusted to 7 with Na₂CO₃. The organic layer was concentrated to give a residue which was further purified by Prep-HPLC to give title product (10.6 mg, 15%). H NMR (500 MHz, CD₃OD) δ 8.86 (d, J=7.8 Hz, 1H), 7.93 (s, 1H), 7.45-7.35 (m, 4H), 7.23 (m, 1H), 7.00 (m, 1H), 5.58 (m, 1H), 5.00 (q, J=6.8 Hz, 1H), 4.61 (m, 2H), 4.57-4.51 (m, 2H), 4.39 (d, J=28.7 Hz, 1H), 3.98 (s, 2H), 3.86 (d, J=11.0 Hz, 1H), 3.79 (m, 1H), 3.76-3.72 (m, 2H), 3.54-3.40 (m, 2H), 2.92 (m, 2H), 2.46 (d, J=5.6 Hz, 3H), 2.21 (m, 3H), 2.06-1.84 (m, 8H), 1.58 (m, 3H), 1.49 (m, 6H), 1.38-1.27 (m, 4H), 1.26-1.18 (m, 2H), 1.02 (d, J=8.4 Hz, 9H). MS (ESI, m/e) [M+1]⁺1112.8.

### Example 48: (2S,4R)-1-((2S)-2-(7-((2S)-2-(((7-(6-amino-4-methyl-3-(trifluoromethyl)pyridin-2-yl)-4-(3,8-diazabicyclo[3.2.1]octan-3-yl)-6-chloro-8-fluoroquinazolin-2-yl)oxy)methyl)pyrrolidin-1-yl)heptanamido)-3,3-dimethylbutanoyl)-4-hydroxy-N—((S)-1-(4-(4-methylthiazol-5-yl)phenyl)ethyl)pyrrolidine-2-carboxamide

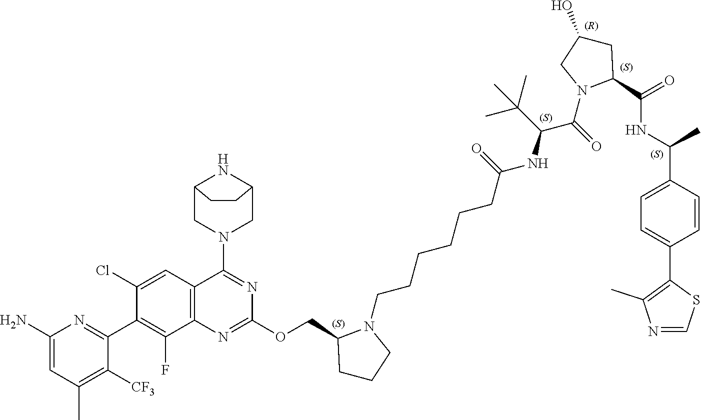

Synthetic Route

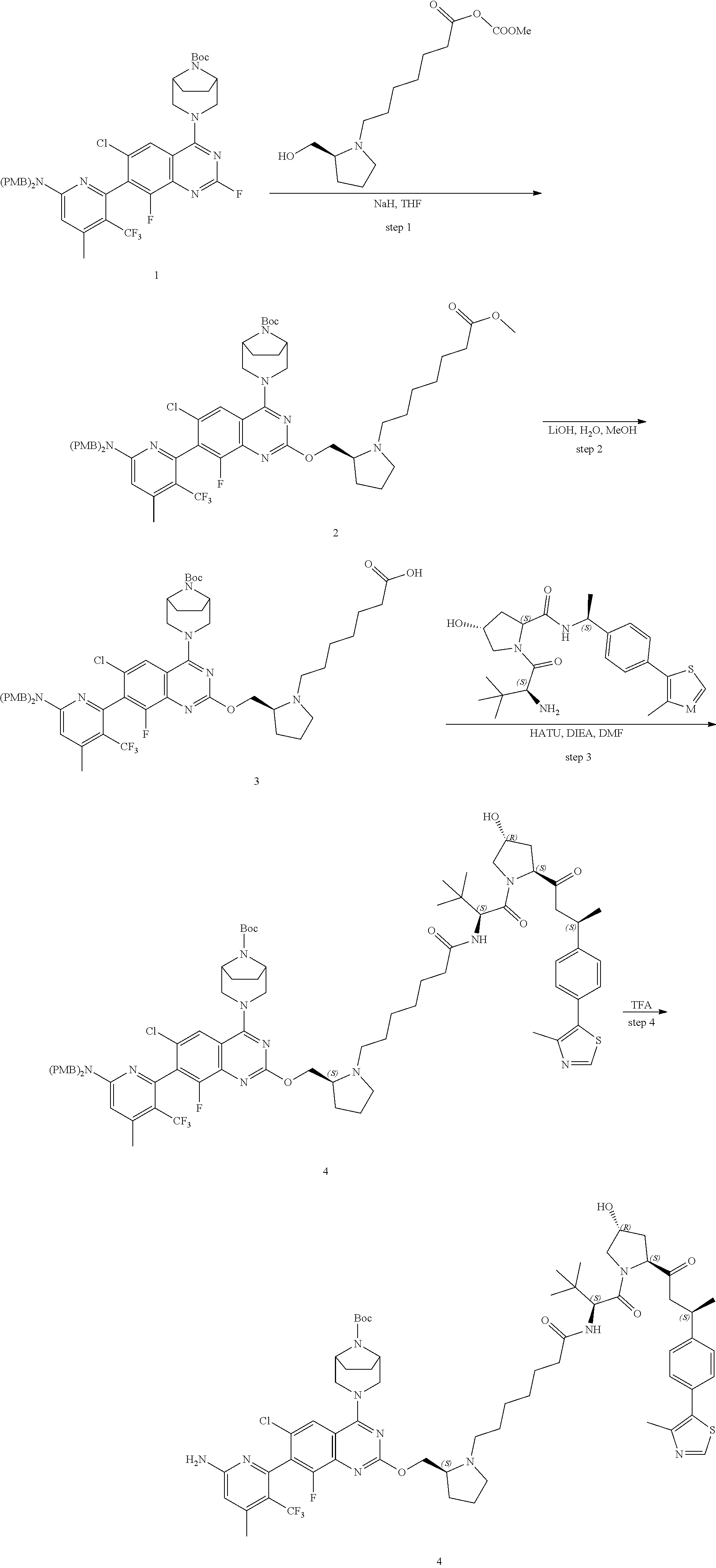

### Step 1: tert-butyl 3-(7-(6-(bis(4-methoxybenzyl)amino)-4-methyl-3-(trifluoromethyl)pyridin-2-yl)-6-chloro-8-fluoro-2-(((S)-1-(7-methoxy-7-oxoheptyl)pyrrolidin-2-yl)methoxy)quinazolin-4-yl)-3,8-diazabicyclo[3.2.1]octane-8-carboxylate

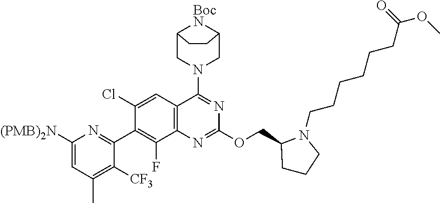

To a solution of methyl (S)-7-(2-(hydroxymethyl)pyrrolidin-1-yl)heptanoate (200 mg, 0.8 mmol) in THF (200 mL) was added NaH (50 mg, 1.2 mmol). The mixture was stirred for 15 mins at room temperature. Then, tert-butyl 3-(7-(6-(bis(4-methoxybenzyl)amino)-4-methyl-3-(trifluoromethyl)pyridin-2-yl)-6-chloro-2,8-difluoroquinazolin-4-yl)-3,8-diazabicyclo[3.2.1]octane-8-carboxylate (500 mg, 0.6 mmol) was added and the reaction mixture was stirred for 8 hrs at room temperature. Upon completion, the mixture was quenched by H₂O and purified by silica column (eluting with DCM/MeOH=9/1) to give the title product (200 mg, 31%). MS (ESI, m/e) [M+1]⁺1048.5.

### Step 2: 7-((2S)-2-(((7-(6-(bis(4-methoxybenzyl)amino)-4-methyl-3-(trifluoromethyl)pyridin-2-yl)-4-(8-(tert-butoxycarbonyl)-3,8-diazabicyclo[3.2.1]octan-3-yl)-6-chloro-8-fluoroquinazolin-2-yl)oxy)methyl)pyrrolidin-1-yl)heptanoic acid

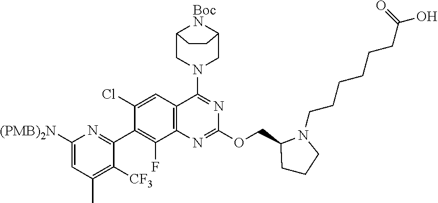

To a solution of tert-butyl 3-(7-(6-(bis(4-methoxybenzyl)amino)-4-methyl-3-(trifluoromethyl)pyridin-2-yl)-6-chloro-8-fluoro-2-(((S)-1-(7-methoxy-7-oxoheptyl)pyrrolidin-2-yl)methoxy)quinazolin-4-yl)-3,8-diazabicyclo[3.2.1]octane-8-carboxylate (200 mg, 0.2 mmol) in MeOH (5 mL) was added LiOH (40 mg, 1 mmol) in H₂O (5 mL), then the mixture was stirred at room temperature for 2.6 hrs. Upon completion, solvent was removed and the residue was purified by chromatography column on silica (eluting with DCM/MeOH=3/2) to give the title product (100 mg, 51%). MS (ESI, m/e) [M+1]⁺1034.6.

### Step 3: tert-butyl 3-(7-(6-(bis(4-methoxybenzyl)amino)-4-methyl-3-(trifluoromethyl)pyridin-2-yl)-6-chloro-8-fluoro-2-(((S)-1-(7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptyl)pyrrolidin-2-yl)methoxy)quinazolin-4-yl)-3,8-diazabicyclo[3.2.1]octane-8-carboxylate

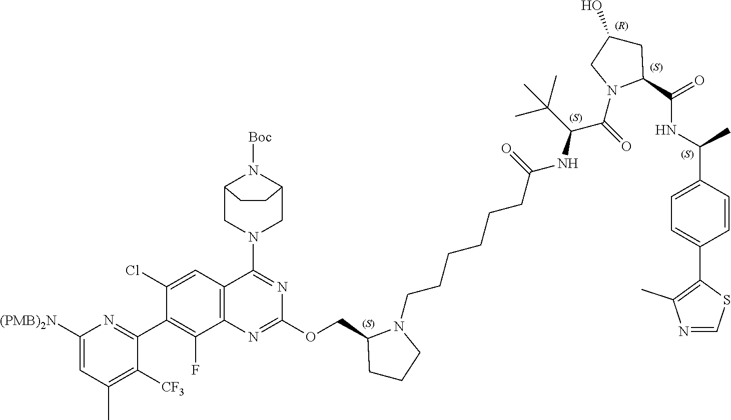

A mixture of 7-((2S)-2-(((7-(6-(bis(4-methoxybenzyl)amino)-4-methyl-3-(trifluoromethyl)pyridin-2-yl)-4-(8-(tert-butoxycarbonyl)-3,8-diazabicyclo[3.2.1]octan-3-yl)-6-chloro-8-fluoroquinazolin-2-yl)oxy)methyl)pyrrolidin-1-yl)heptanoic acid (100 mg, 0.10 mmol), (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrogen chloride (50 mg, 0.11 mmol), DIEA (0.3 mL), and DMF (6 mL) was stirred at room temperature for 10 mins. Then HATU (50 mg, 0.13 mmol) was added and the mixture was stirred for 3 hrs. The mixture was extracted with DCM/MeOH=9/1 and combined organic layer was concentrated. The residue was purified by column chromatography (DCM/MeOH=10/1) to give the title product (80 mg, 57%). MS (ESI, m/e) [M+1]⁺1460.8.

### Step 4: (2S,4R)-1-((2S)-2-(7-((2S)-2-(((7-(6-amino-4-methyl-3-(trifluoromethyl)pyridin-2-yl)-4-(3,8-diazabicyclo[3.2.1]octan-3-yl)-6-chloro-8-fluoroquinazolin-2-yl)oxy)methyl)pyrrolidin-1-yl)heptanamido)-3,3-dimethylbutanoyl)-4-hydroxy-N—((S)-1-(4-(4-methylthiazol-5-yl)phenyl)ethyl)pyrrolidine-2-carboxamide

A mixture of tert-butyl 3-(7-(6-(bis(4-methoxybenzyl)amino)-4-methyl-3-(trifluoromethyl)pyridin-2-yl)-6-chloro-8-fluoro-2-(((S)-1-(7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptyl)pyrrolidin-2-yl)methoxy)quinazolin-4-yl)-3,8-diazabicyclo[3.2.1]octane-8-carboxylate (80 mg, 0.05 mmol) and TFA (6 mL) was stirred at 40° C. for 6 hrs. Upon completion, Solvent was removed and adjusted to pH=8 with MeOH/NH₃. Then the mixture was concentrated to give a residue which was further purified by Prep-HPLC to give the title product (2.35 mg, 4%). ¹H NMR (500 MHz, DMSO-d₆) δ 8.88 (s, 1H), 8.47 (s, 1H), 7.89 (s, 1H), 7.43-7.40 (m, 4H), 6.61 (s, 1H), 5.03-4.96 (m, 1H), 4.89-4.83 (m, 1H), 4.65-4.50 (m, 4H), 4.45-4.41 (m, 1H), 3.98-3.92 (m, 2H), 3.78-3.74 (m, 2H), 3.64-3.46 (m, 3H), 3.43-3.35 (m, 1H), 3.52-3.45 (m, 1H), 3.45-3.16 (m, 2H), 2.47-2.38 (m, 6H), 2.44-2.27 (m, 4H), 2.20-1.92 (m, 8H), 1.748-1.72 (m, 2H), 1.68-1.54 (m, 2H), 1.51-1.18 (m, 3H), 1.43-1.34 (m, 4H), 1.09-0.97 (m, 10H). MS (ESI, m/e) [M+H]⁺ 1120.80.

### Example 49: 6-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-4-methyl-5-(trifluoromethyl)pyridin-2-amine

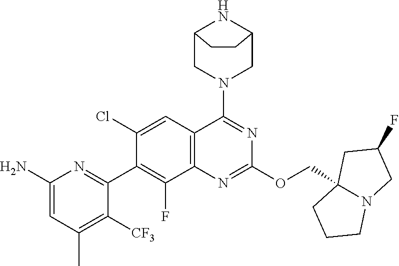 [SMILES: Cc1cc(N)nc(-c2c(Cl)cc3c(N4CC5CCC(C4)N5)nc(OC[C@@]45CCCN4C[C@H](F)C5)nc3c2F)c1C(F)(F)F]

### Step 1: tert-butyl-3-(7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

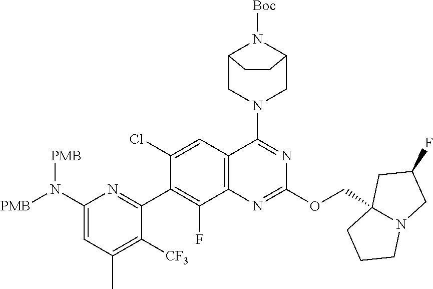

To a solution of ((2R,7aS)-2-fluorotetrahydro-1H-pyrrolizin-7a(5H)-yl)methanol (110 mg, 0.69 mmol) in THF (15 mL) was added NaH (100 mg). The mixture was stirred for 15 mins at room temperature and then tert-butyl-3-(7-(6-(bis(4-methoxybenzyl)amino)-4-methyl-3-(trifluoromethyl)pyridin-2-yl)-6-chloro-2,8-difluoroquinazolin-4-yl)-3,8-diazabicyclo[3.2.1]octane-8-carboxylate (200 mg, 0.24 mmol) was added. The reaction mixture was stirred for 3 hours at room temperature. After completion, the reaction mixture was quenched by H₂O and solvents were removed. Crude product was purified by silica column (eluting with DCM/MeOH=10/1) to give the title product (150 mg, 64%). MS (ESI, m/e) [M+1]⁺964.3.

### Step 2: 6-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-4-methyl-5-(trifluoromethyl)pyridin-2-amine

A solution of tert-butyl-3-(7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (150 mg, 0.156 mmol) in TFA (6 mL) was stirred for 16 hours at 40° C. After completion, the reaction mixture was concentrated, then adjusted pH to 9 with DIPEA. The crude product was purified by C18 column (eluting with H₂O/CH₃CN=4/1) to afford the title product (54 mg, FA salt). ¹H NMR (500 MHz, CD₃OD) δ 8.42 (s, 2H), 7.89 (s, 1H), 6.61 (s, 1H), 5.42-5.52 (m, 1H), 4.62-4.47 (m, 4H), 4.03 (s, 2H), 3.77-3.60 (m, 5H), 2.57-1.92 (m, 14H). MS (ESI, m/e) [M+H]⁺ 624.3.

### Example 50: 6-(4-(3,8-diazabicyclo[3.2.1]octan-3-yl)-6-chloro-8-fluoro-2-((2-fluorotetrahydro-1H-pyrrolizin-7a(5H)-yl)methoxy)quinazolin-7-yl)-4-methyl-5-(trifluoromethyl)pyridin-2-amine

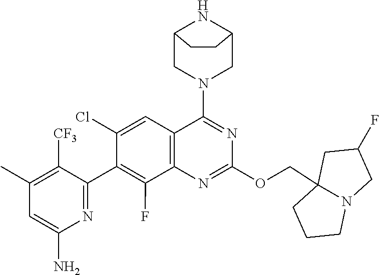 [SMILES: Cc1cc(N)nc(-c2c(Cl)cc3c(N4CC5CCC(C4)N5)nc(OCC45CCCN4CC(F)C5)nc3c2F)c1C(F)(F)F]

Example 50 (TFA salt) was prepared by similar procedure as described in Example 49 by replacing ((2R,7aS)-2-fluorotetrahydro-1H-pyrrolizin-7a(5H)-yl)methanol with (2-fluorotetrahydro-1H-pyrrolizin-7a(5H)-yl)methanol. ¹H NMR (500 MHz, CD₃OD) δ 7.94 (s, 1H), 6.67 (s, 1H), 5.68-5.45 (m, 1H), 4.79-4.64 (m, 4H), 4.30-4.20 (m, 2H), 4.01-3.80 (m, 5H), 2.80-2.15 (m, 14H). MS (ESI, m/e) [M+H]⁺ 624.1.

### Example 51: 4-(4-(3,8-diazabicyclo[3.2.1]octan-3-yl)-8-fluoro-2-(((2S,4R)-4-fluoro-1-methylpyrrolidin-2-yl)methoxy)-6-(trifluoromethyl)quinazolin-7-yl)-7-fluorobenzo[d]thiazol-2-amine

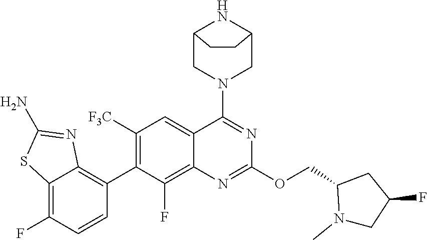 [SMILES: CN1C[C@H](F)C[C@H]1COc1nc(N2CC3CCC(C2)N3)c2cc(C(F)(F)F)c(-c3ccc(F)c4sc(N)nc34)c(F)c2n1]

### Step 1: methyl 2-amino-4-bromo-3-fluorobenzoate

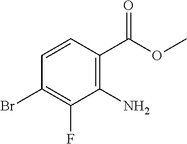

To a solution of 2-amino-4-bromo-3-fluorobenzoic acid (37.0 g, 158 mmol) in methanol (300 mL) was added sulfurous dichloride (50 mL) at 0° C. . The reaction mixture was then stirred at reflux for 16 hrs. After completion, solvents were evaporated and the residue was mixed with dichloromethane and water. The organic layer was dried over sodium sulfate and evaporated. The residue was purified by column chromatography on silica to give the title compound (36.0 g, 91%). MS (ESI, m/e) [M+1]⁺247.9.

### Step 2: methyl 2-amino-4-bromo-3-fluoro-5-iodobenzoate

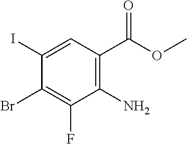

To a solution of methyl 2-amino-4-bromo-3-fluorobenzoate (36.0 g, 144.5 mmol) in ethanol (1.0 L) was added iodine (73.7 g, 290.7 mmol) and silver sulfate (59.0 g, 189.2 mmol) and the reaction mixture was stirred at room temperature for 16 hrs. Then it was filtered, and the filtrate was concentrated in vacuo. The residue was purified by column chromatography on silica to give the title compound (35.0 g, 65%). MS (ESI, m/e) [M+H]⁺ 373.9.

### Step 3: methyl 2-acetamido-4-bromo-3-fluoro-5-iodobenzoate

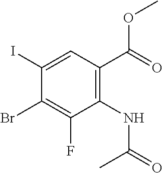 [SMILES: COC(=O)c1cc(I)c(Br)c(F)c1NC(C)=O]

To a solution of methyl 2-amino-4-bromo-3-fluoro-5-iodobenzoate (35.0 g, 93.8 mmol) in dichloromethane (500 mL) was added pyridine (14.0 g, 177.0 mmol) at 0° C. and acetyl chloride (14.6 g, 186.0 mmol) was added dropwise. After the addition, the reaction mixture was stirred at room temperature for 16 hrs. Then it was washed with brine, and the organic layer was dried over sodium sulfate and evaporated. The residue was purified by flash column on silica gel to give the title compound (22.0 g, 56%). MS (ESI, m/e) [M+H]⁺ 415.9.

### Step 4: methyl 2-acetamido-4-bromo-3-fluoro-5-(trifluoromethyl)benzoate

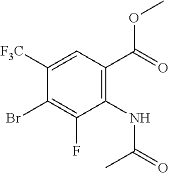 [SMILES: COC(=O)c1cc(C(F)(F)F)c(Br)c(F)c1NC(C)=O]

To a solution of methyl 2-acetamido-4-bromo-3-fluoro-5-iodobenzoate (8.50 g, 20.4 mmol) in N-methyl pyrrolidone (90 mL) was added methyl 2,2-difluoro-2-(fluorosulfonyl)acetate (8.0 g, 41.6 mmol) and cuprous iodide (1.20 g, 6.30 mmol) at room temperature. After the addition, the mixture was stirred at 80° C. for 6 hrs. Then it was cooled to room temperature and diluted with ethyl acetate and water. The combined organic layer rotovaped and the residue was purified by column chromatography on silica gel to give the title compound (4.5 g, 59%). MS (ESI, m/e) [M+H]⁺ 358.0.

### Step 5: 2-acetamido-4-bromo-3-fluoro-5-(trifluoromethyl)benzoic acid

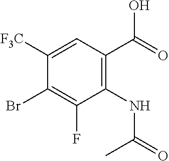 [SMILES: CC(=O)Nc1c(C(=O)O)cc(C(F)(F)F)c(Br)c1F]

To a solution of methyl 2-acetamido-4-bromo-3-fluoro-5-(trifluoromethyl)benzoate (4.50 g, 10.8 mmol) in tetrahydrofuran (20 mL) was added lithium hydroxide (20 mL, 1M in water). After the addition, the mixture was stirred at room temperature for 6 hrs. pH was adjusted to 3 with hydrogen chloride in water (2 N). The mixture was diluted with ethyl acetate and water. Combined organic layer was dried over sodium sulfate and evaporated to give the crude title compound (2.50 g) which was used directly in the next step. MS (ESI, m/e) [M+H]⁺ 344.0.

### Step 6: 2-amino-4-bromo-3-fluoro-5-(trifluoromethyl)benzoic acid

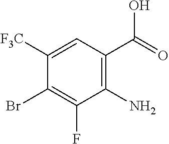 [SMILES: Nc1c(C(=O)O)cc(C(F)(F)F)c(Br)c1F]

To a solution of 2-acetamido-4-bromo-3-fluoro-5-(trifluoromethyl)benzoic acid (2.50 g, 7.31 mmol) in methanol (15 mL) was added hydrogen chloride in isopropanol (15 mL, 4M). After the addition, the mixture was stirred at room temperature for 8 hrs. Then, solvent was evaporated to give the crude title compound (1.60 g) which was used directly in the next step. MS (ESI, m/e) [M+H]⁺ 301.9.

### Step 7: 7-bromo-8-fluoro-6-(trifluoromethyl)quinazoline-2,4-diol

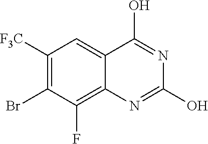 [SMILES: Oc1nc(O)c2cc(C(F)(F)F)c(Br)c(F)c2n1]

2-amino-4-bromo-3-fluoro-5-(trifluoromethyl)benzoic acid (1.60 g, 5.29 mmol) and urea (12.5 g, 208 mmol) was stirred at 190° C. overnight. More urea (2.5 g, 41 mmol) was added and the mixture was stirred for additional 4 hrs at 190° C. Then, the mixture was cooled to 60° C. and hot water (60° C., 50 mL) was added and the mixture was stirred at room temperature for 0.5 h. Solid was filtered and washed with dichloromethane:methanol=20:1 and the aqueous layer was also washed with dichloromethane:methanol=20:1. Combined organic layer was evaporated and the residue was dissolved in dichloromethane:ethyl acetate:petroleum ether=1:1:20 solvent. Solid was filtered and washed with dichloromethane:ethyl acetate=1:1 solvent for 5 times. Combined organic layer was dried over sodium sulfate and evaporate. The residue was purified by column chromatography on silica to give to give the title compound (300 mg, 17%). MS (ESI, m/e) [M+H]⁺ 326.9.

### Step 8: 7-bromo-2,4-dichloro-8-fluoro-6-(trifluoromethyl)quinazoline

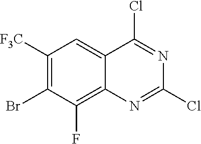 [SMILES: Fc1c(Br)c(C(F)(F)F)cc2c(Cl)nc(Cl)nc12]

To a solution of 7-bromo-8-fluoro-6-(trifluoromethyl)quinazoline-2,4-diol (360 mg, 1.10 mmol) in phosphorus oxychloride (6 mL) was added DIPEA (1.30 g, 10.0 mmol) at room temperature. After the addition, the mixture was stirred at 100° C. for 4 hrs. The mixture was cooled to room temperature and poured into ice water. The mixture was diluted with dichloromethane and water. Combined organic layer was dried over sodium sulfate and evaporated to give the title compound (360 mg) which was used directly in the next step. MS (ESI, m/e) [M+H]⁺ 363.0.

### Step 9: tert-butyl-3-(7-bromo-2-chloro-8-fluoro-6-(trifluoromethyl)quinazolin-4-yl)-3,8-diazabicyclo[3.2.1]octane-8-carboxylate

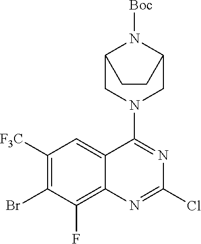 [SMILES: CC(C)(C)OC(=O)N1C2CCC1CN(c1nc(Cl)nc3c(F)c(Br)c(C(F)(F)F)cc13)C2]

To a solution of 7-bromo-2,4-dichloro-8-fluoro-6-(trifluoromethyl)quinazoline (360 mg, 0.99 mmol) in dichloromethane (5 mL) was added DIPEA (1 mL) and tert-butyl-3,8-diazabicyclo[3.2.1]octane-8-carboxylate (210 mg, 0.99 mmol) at 0° C. After the addition, the mixture was stirred at 0° C. for 1 h. Then the mixture was diluted with dichloromethane and water. Combined organic layer was dried over sodium sulfate and evaporated. The residue was purified by column chromatography on silica to give the title compound (300 mg, 56%). MS (ESI, m/e) [M+H]⁺ 539.2.

### Step 10: tert-butyl-3-(7-bromo-2,8-difluoro-6-(trifluoromethyl)quinazolin-4-yl)-3,8-diazabicyclo[3.2.1]octane-8-carboxylate

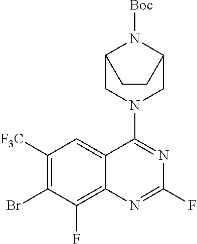 [SMILES: CC(C)(C)OC(=O)N1C2CCC1CN(c1nc(F)nc3c(F)c(Br)c(C(F)(F)F)cc13)C2]

To a solution of tert-butyl-3-(7-bromo-2-chloro-8-fluoro-6-(trifluoromethyl)quinazolin-4-yl)-3,8-diazabicyclo[3.2.1]octane-8-carboxylate (300 mg, 0.56 mmol) in dimethylsulfoxide (5 mL) was added potassium fluoride (290 mg, 5.0 mmol). After the addition, the mixture was stirred at 100° C. for 4.5 hrs. Then it was cooled to room temperature and diluted with dichloromethane and water. Combined organic layer was dried over sodium sulfate and evaporated. The residue was purified by column chromatography on silica to give the title compound (200 mg, 68%). MS (ESI, m/e) [M+H]⁺ 523.1.

### Step 11: tert-butyl-3-(7-(2-((tert-butoxycarbonyl)amino)-7-fluorobenzo[d]thiazol-4-yl)-2,8-difluoro-6-(trifluoromethyl)quinazolin-4-yl)-3,8-diazabicyclo[3.2.1]octane-8-carboxylate

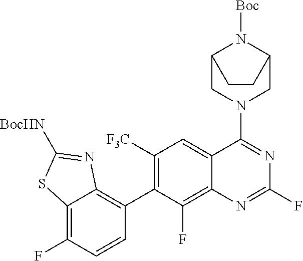 [SMILES: CC(C)(C)OC(=O)Nc1nc2c(-c3c(C(F)(F)F)cc4c(N5CC6CCC(C5)N6C(=O)OC(C)(C)C)nc(F)nc4c3F)ccc(F)c2s1]

To a solution of tert-butyl-3-(7-bromo-2,8-difluoro-6-(trifluoromethyl)quinazolino-4-yl)-3,8-diazabicyclo[3.2.1]octane-8-carboxylate (175 mg, 0.33 mmol) in dioxane:water (10 mL: 2 mL) was added (2-((tert-butoxycarbonyl)amino)-7-fluorobenzo[d]thiazol-4-yl)boronic acid (130 mg, 0.42 mmol), 1,1′-bis(diphenylphosphino)ferrocene-palladium(II)dichloride dichloromethane complex (26 mg, 0.03 mmol) and sodium bicarbonate (80 mg, 0.95 mmol) at room temperature. After the addition, the mixture was stirred at 90° C. for 1.5 hrs. Then it was cooled to room temperature, and the mixture was diluted with dichloromethane and water. Combined organic layer was dried over sodium sulfate and evaporated. The residue was purified by column chromatography on silica to give the title compound (100 mg, 42%). MS (ESI, m/e) [M+H]⁺ 711.3.

### Step 12: tert-butyl-3-(7-(2-((tert-butoxycarbonyl)amino)-7-fluorobenzo[d]thiazol-4-yl)-8-fluoro-2-(((2S,4R)-4-fluoro-1-methylpyrrolidin-2-yl)methoxy)-6-(trifluoromethyl)quinazolin-4-yl)-3,8-diazabicyclo[3.2.1]octane-8-carboxylate

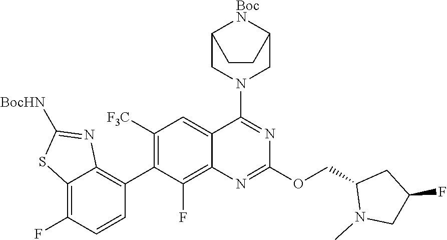 [SMILES: CN1C[C@H](F)C[C@H]1COc1nc(N2CC3CCC(C2)N3C(=O)OC(C)(C)C)c2cc(C(F)(F)F)c(-c3ccc(F)c4sc(NC(=O)OC(C)(C)C)nc34)c(F)c2n1]

To a solution of ((2S,4R)-4-fluoro-1-methylpyrrolidin-2-yl)methanol (40 mg, 0.30 mmol) was added sodium hydride (60%) (20 mg, 0.49 mmol) at 0° C. and the mixture was stirred at room temperature for 0.5 h. Then tert-butyl-3-(7-(2-((tert-butoxycarbonyl)amino)-7-fluorobenzo[d]thiazol-4-yl)-8-fluoro-2-(((2S,4R)-4-fluoro-1-methylpyrrolidin-2-yl)methoxy)-6-(trifluoromethyl)quinazolin-4-yl)-3,8-diazabicyclo[3.2.1]octane-8-carboxylate (50 mg, 0.07 mmol) in tetrahydrofuran (1 mL) was added dropwise. The mixture was stirred at room temperature for another 1 h and then quenched with aq ammonium chloride at 0° C. The mixture was diluted with dichloromethane and water and the organic layer was combined, dried over sodium sulfate and evaporated. The residue was purified by preparative thin layer chromatography on silica to give the title compound (20 mg, 35%). MS (ESI, m/e) [M+H]⁺ 824.5.

### Step 13: 4-(4-(3,8-diazabicyclo[3.2.1]octan-3-yl)-8-fluoro-2-(((2S,4R)-4-fluoro-1-methylpyrrolidin-2-yl)methoxy)-6-(trifluoromethyl)quinazolin-7-yl)-7-fluorobenzo[d]thiazol-2-amine

To a solution of tert-butyl-3-(7-(2-((tert-butoxycarbonyl)amino)-7-fluorobenzo[d]thiazol-4-yl)-8-fluoro-2-(((2S,4R)-4-fluoro-1-methylpyrrolidin-2-yl)methoxy)-6-(trifluoromethyl)quinazolin-4-yl)-3,8-diazabicyclo[3.2.1]octane-8-carboxylate (20 mg, 0.024 mmol) in dichloromethane (2 mL) was added trifluoroacetic acid (1 mL) at room temperature. After the addition, the mixture was stirred at room temperature for 4 hrs. Then the solution was evaporated, and the residue was adjusted to pH=10 with aq sodium carbonate. The mixture was mixed with dichloromethane:methanol=10:1, and the organic layer was dried over sodium sulfate and evaporated. The residue was lyophilized to give the title compound (7 mg, 46%). ¹H NMR (500 MHz, CD₃OD) δ 8.12 (s, 1H), 7.22-7.16 (m, 1H), 6.99-6.92 (m, 1H), 5.26-5.09 (m, 1H), 4.58-4.44 (m, 4H), 3.75-3.61 (m, 4H), 3.55-3.43 (m, 1H), 3.18-3.10 (m, 1H), 2.71-2.59 (m, 1H), 2.55 (s, 3H), 2.34-2.22 (m, 1H), 2.06-1.92 (m, 1H), 1.86-1.74 (m, 4H). MS (ESI, m/e) [M+H]⁺ 624.4.

### Example 52: 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzoI[d]thiazol-2-amine

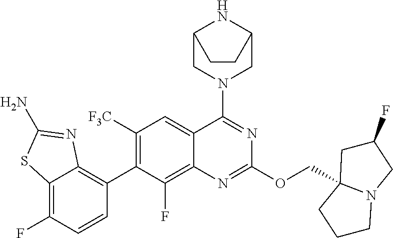 [SMILES: Nc1nc2c(-c3c(C(F)(F)F)cc4c(N5CC6CCC(C5)N6)nc(OC[C@@]56CCCN5C[C@H](F)C6)nc4c3F)ccc(F)c2s1]

### Step 1: tert-butyl-3-(7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate

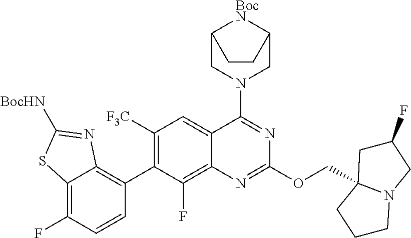 [SMILES: CC(C)(C)OC(=O)Nc1nc2c(-c3c(C(F)(F)F)cc4c(N5CC6CCC(C5)N6C(=O)OC(C)(C)C)nc(OC[C@@]56CCCN5C[C@H](F)C6)nc4c3F)ccc(F)c2s1]

To a solution of ((2R,7aS)-2-fluorotetrahydro-1H-pyrrolizin-7a(5H)-yl)methanol (67.18 mg, 0.42 mmol) in THF (5 mL) was added sodium hydride (16.19 mg, 0.42 mmol) at 0° C. The resulting mixture was stirred at 0° C. for 1 h. Then tert-butyl 3-(7-(2-((tert-butoxycarbonyl)amino)-7-fluorobenzo[d]thiazol-4-yl)-2,8-difluoro-6-(trifluoromethyl)quinazolin-4-yl)-3,8-diazabicyclo[3.2.1]octane-8-carboxylate (100 mg, 0.14 mmol) was added to the reaction mixture and stirred for 2 hrs at room temperature. After completion, the reaction mixture was quenched with water (20 mL), extracted with DCM (50 mL×3), and the organic layers was dried over anhydrous Na₂SO₄, filtered and the filtrate was concentrated under reduced pressure to give the crude title product (150 mg). MS (ESI, m/e) [M+1]⁺850.3.

### Step 2: 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

To a solution of tert-butyl-3-(7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate (150 mg) in DCM (10 mL) was added TFA (2 mL) at room temperature. The resulting solution was stirred for 4 hrs at room temperature. After completion, the reaction mixture was concentrated to give a residue, which was purified by reverse phase prep-HPLC at room temperature with the following conditions: Column: waters sunfire C18 gel column (19×150 mm), 5 um; Mobile Phase A: 1% aq. HCOOH; Mobile Phase B: MeCN; (Gradient: 17% B hold 11 min, then increased to 90% B within 0.2 min, and 90% B hold 5 min); Flow rate: 17 mL/min; Detector: UV 254 & 280 nm; retention time: 6.8 min. The fractions containing product were collected and lyophilized overnight to give the title product (7.3 mg, FA salt). ¹H NMR (500 MHz, CD₃OD) δ 8.42 (s, 1H), 8.15 (s, 1H), 7.20-7.17 (m, 1H), 6.99-6.96 (m, 1H), 5.52-5.42 (m, 1H), 4.67-4.65 (m, 2H), 4.55-4.51 (m, 2H), 4.04 (s, 2H), 3.87-3.67 (m, 5H), 3.34-3.33 (m, 1H), 2.55-2.45 (m, 2H), 2.33-2.31 (m, 1H), 2.22-2.19 (m, 2H), 2.05-1.97 (m, 5H). MS (ESI, m/e) [M+H]⁺ 650.3.

### Example 53: 4-(4-(3,8-diazabicyclo[3.2.1]octan-3-yl)-6,8-dichloro-2-(((2S,4R)-4-fluoro-1-methylpyrrolidin-2-yl)methoxy)quinazolin-7-yl)-7-fluorobenzo[d]thiazol-2-amine

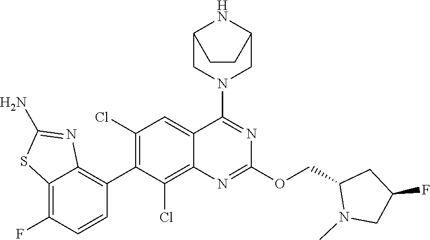 [SMILES: CN1C[C@H](F)C[C@H]1COc1nc(N2CC3CCC(C2)N3)c2cc(Cl)c(-c3ccc(F)c4sc(N)nc34)c(Cl)c2n1]

### Step 1: N-(3-bromo-2-chlorophenyl)-2-(hydroxyimino)acetamide

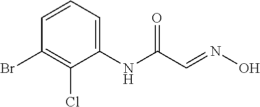 [SMILES: O=C(/C=N/O)Nc1cccc(Br)c1Cl]

To a mixture of 3-bromo-2-chloroaniline (30 g, 0.146 mol) in water (300 mL) and HCl (30 mL) was added hydroxylamine hydrochloride (30 g, 0.435 mol) and 2,2,2-trichloroethane-1,1-diol (30 g, 0.183 mol) stepwise. The mixture was stirred for 6 hours at 100° C. After completion, the resulting mixture was cooled, and the solid was filtered and collected as the crude product (20 g) which was used directly in next step. MS (ESI, m/e) [M+1]⁺276.9.

### Step 2: 6-bromo-7-chloroindoline-2,3-dione

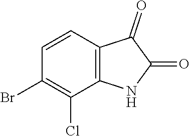 [SMILES: O=C1Nc2c(ccc(Br)c2Cl)C1=O]

A solution of N-(3-bromo-2-chlorophenyl)-2-(hydroxyimino)acetamide (20 g, 72 mmol) in H₂SO₄ (100 mL) was stirred for 4 hours at 100° C. The resulting solution was cooled and poured into ice-water, and extracted with EtOAc. The combined organic layers was concentrated to give crude product (16 g) which was used directly in the next step. MS (ESI, m/e) [M+1]⁺259.9.

### Step 3: 2-amino-4-bromo-3-chlorobenzoic acid

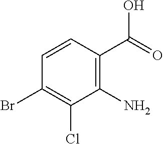 [SMILES: Nc1c(C(=O)O)ccc(Br)c1Cl]

To a mixture of 6-bromo-7-chloroindoline-2,3-dione (16 g, 62 mmol) in NaOH (300 mL, 155 mmol) was added H₂O₂ (14 mL) and the mixture was stirred for 4 hours at room temperature. After completion, the resulting mixture was extracted with EtOAc. The aqueous layer was adjusted to pH=3 with HCl (aq). The solid was filtered and collected to give the title product (5.6 g). MS (ESI, m/e) [M+1]⁺249.9.

### Step 4: 2-amino-4-bromo-3,5-dichlorobenzoic acid

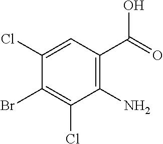 [SMILES: Nc1c(C(=O)O)cc(Cl)c(Br)c1Cl]

To a mixture of 2-amino-4-bromo-3-chlorobenzoic acid (5.6 g, 22 mmol) in DMF (80 mL) was added N-chlorosuccinimide (3.9 g, 29 mmol) and the mixture was stirred for 6 hours at 70° C. After completion, the resulting mixture was poured into water and extracted with EtOAc. The organic layer was combined and concentrated. Then the crude product was further purified by C18 column (eluting with H₂O/CH₃CN=1/1) to afford the title product (2 g, 32%). MS (ESI, m/e) [M+1]⁺284.1.

### Step 5: 7-bromo-6,8-dichloroquinazoline-2,4-diol

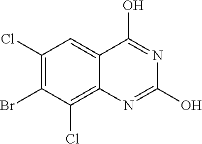 [SMILES: Oc1nc(O)c2cc(Cl)c(Br)c(Cl)c2n1]

A mixture of 2-amino-4-bromo-3,5-dichlorobenzoic acid (2 g, 7 mmol) and urea (10 g) was stirred for 8 hours at 150° C. After completion, the resulting mixture was poured into hot water (80° C.) and filtered. The solids were further purified by silica column (eluting with DCM/MeOH=19/1) to afford the title product (1.2 g, 56%). MS (ESI, m/e) [M+1]⁺309.1.

### Step 6: 7-bromo-2,4,6,8-tetrachloroquinazoline

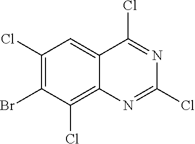 [SMILES: Clc1nc(Cl)c2cc(Cl)c(Br)c(Cl)c2n1]

To a mixture of 7-bromo-6,8-dichloroquinazoline-2,4-diol (1.2 g, 3.9 mmol) in POCl₃ (16 mL) was added DIPEA (4 mL) and the mixture was stirred for 3 hours at 120° C. After completion, the resulting mixture was poured into ice-water and extracted with DCM. The organic layer was concentrated, and the crude product was further purified by silica column (eluting with DCM/MeOH=19/1) to afford the title product (700 mg, 51%). MS (ESI, m/e) [M+1]⁺344.8.

### Step 7: tert-butyl-3-(7-bromo-2,6,8-trichloroquinazolin-4-yl)-3,8-diazabicyclo[3.2.1]octane-8-carboxylate

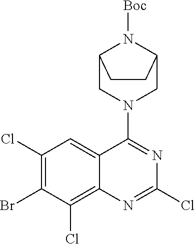 [SMILES: CC(C)(C)OC(=O)N1C2CCC1CN(c1nc(Cl)nc3c(Cl)c(Br)c(Cl)cc13)C2]

A mixture of 7-bromo-2,4,6,8-tetrachloroquinazoline (700 mg, 2 mmol), tert-butyl-3,8-diazabicyclo[3.2.1]octane-8-carboxylate (431 mg, 2 mmoL) in DCM (20 mL) and DIPEA (1 mL) was stirred for 2 hours at room temperature. After completion, solvents were evaporated and the crude product was purified by silica column (eluting with PE/EtOAc=1/1) to afford the title product (700 mg, 65%). MS (ESI, m/e) [M+1]⁺520.3.

### Step 8: tert-butyl-3-(7-(2-((tert-butoxycarbonyl)amino)-7-fluorobenzo[d]thiazol-4-yl)-2,6,8-trichloroquinazolin-4-yl)-3,8-diazabicyclo [3.2.1]octane-8-carboxylate

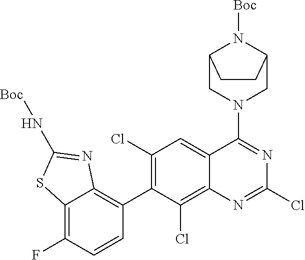 [SMILES: CC(C)(C)OC(=O)Nc1nc2c(-c3c(Cl)cc4c(N5CC6CCC(C5)N6C(=O)OC(C)(C)C)nc(Cl)nc4c3Cl)ccc(F)c2s1]

To a solution of tert-butyl-3-(7-bromo-2,6,8-trichloroquinazolin-4-yl)-3,8-diazabicyclo[3.2.1]octane-8-carboxylate (700 mg, 1.3 mmol) in dioxane (20 mL) and H₂O (2 mL) was added Pd(dppf)Cl₂ (100 mg), NaHCO₃ (400 mg) and (2-((tert-butoxycarbonyl)amino)-7-fluorobenzo[d]thiazol-4-yl)boronic acid (630 mg, 0.195 mol) stepwise. The reaction mixture was stirred for 3 h at 100° C. After completion, solvents were evaporated and the crude product was purified by silica column (eluting with PE/EtOAc=3/2) to give the title product (380 mg, 41%). MS (ESI, m/e) [M+1]⁺709.2.

### Step 9: tert-butyl-3-(7-(2-((tert-butoxycarbonyl)amino)-7-fluorobenzo[d]thiazol-4-yl)-6,8-dichloro-2-fluoroquinazolin-4-yl)-3,8-diazabicyclo[3.2.1]octane-8-carboxylate

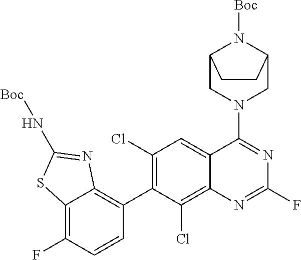 [SMILES: CC(C)(C)OC(=O)Nc1nc2c(-c3c(Cl)cc4c(N5CC6CCC(C5)N6C(=O)OC(C)(C)C)nc(F)nc4c3Cl)ccc(F)c2s1]

A mixture of tert-butyl-3-(7-(2-((tert-butoxycarbonyl)amino)-7-fluorobenzo[d]thiazol-4-yl)-2,6,8-trichloroquinazolin-4-yl)-3,8-diazabicyclo[3.2.1]octane-8-carboxylate (300 mg, 0.42 mmol) in DMSO (10 mL), then added KF (300 mg) into the solution and stirred for 1 day at 100° C. After completion, the resulting mixture was poured into water and extracted with EtOAc. The organic layer was combined and concentrated. Then the crude product was further purified by silica column (eluting with PE/EtOAc=3/2) to give the title product (140 mg, 48%). MS (ESI, m/e) [M+1]⁺693.3.

### Step 10: tert-butyl-3-(7-(2-((tert-butoxycarbonyl)amino)-7-fluorobenzo[d]thiazol-4-yl)-6,8-dichloro-2-(((2S,4R)-4-fluoro-1-methylpyrrolidin-2-yl)methoxy)quinazolin-4-yl)-3,8-diazabicyclo[3.2.1]octane-8-carboxylate

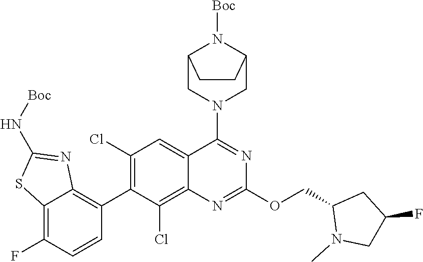 [SMILES: CN1C[C@H](F)C[C@H]1COc1nc(N2CC3CCC(C2)N3C(=O)OC(C)(C)C)c2cc(Cl)c(-c3ccc(F)c4sc(NC(=O)OC(C)(C)C)nc34)c(Cl)c2n1]

To a solution of ((2S,4R)-4-fluoro-1-methylpyrrolidin-2-yl)methanol (50 mg, 0.37 mmol) in THF (10 mL) was added NaH (50 mg). The mixture was stirred for 15 mins at room temperature and then tert-butyl-3-(7-(2-((tert-butoxycarbonyl)amino)-7-fluorobenzo[d]thiazol-4-yl)-6,8-dichloro-2-fluoroquinazolin-4-yl)-3,8-diazabicyclo[3.2.1]octane-8-carboxylate (140 mg, 0.2 mmol) was added. The reaction mixture was stirred for 3 hours at 60° C. After completion, the reaction was quenched by H₂O. Solvents were evaporated and the crude product was purified by silica column (eluting with DCM/MeOH=10/1) to give the title product (80 mg, 49%). MS (ESI, m/e) [M+1]⁺806.2.

### Step 11: 4-(4-(3,8-diazabicyclo[3.2.1]octan-3-yl)-6,8-dichloro-2-(((2S,4R)-4-fluoro-1-methylpyrrolidin-2-yl)methoxy)quinazolin-7-yl)-7-fluorobenzo[d]thiazol-2-amine

A mixture of tert-butyl-3-(7-(2-((tert-butoxycarbonyl)amino)-7-fluorobenzo[d]thiazol-4-yl)-6,8-dichloro-2-(((2S,4R)-4-fluoro-1-methylpyrrolidin-2-yl)methoxy)quinazolin-4-yl)-3,8-diazabicyclo[3.2.1]octane-8-carboxylate (80 mg, 0.1 mmol) in DCM (4 mL) and TFA (2 mL) was stirred for 2 hours at room temperature. After completion, adjusted pH=9 with DIPEA. Then the crude product was purified by C18 column (eluting with H₂O/CH₃CN=3/1) to afford the title product (52 mg, FA salt). ¹H NMR (500 MHz, CD₃OD) δ 8.44 (s, 1H), 8.00 (s, 1H), 7.12 (dd, J=2.8 10 Hz, 1H), 7.00 (t, 3H), 5.28-5.18 (m, 1H), 4.16-4.51 (m, 4H), 4.11 (s, 2H), 3.83-3.76 (m, 2H), 3.64-3.58 (m, 1H), 3.34-3.33 (m, 1H), 2.88-2.80 (m, 1H), 2.67 (s, 3H), 2.40-2.30 (m, 1H), 2.18-2.20 (m, 5H). MS (ESI, m/e) [M+H]⁺ 605.3.

### Example 54: 4-(4-(3,8-diazabicyclo[3.2.1]octan-3-yl)-6-chloro-8-fluoro-2-((2-fluorotetrahydro-1H-pyrrolizin-7a(5H)-yl)methoxy)quinazolin-7-yl)-7-fluorobenzo[d]thiazol-2-amine

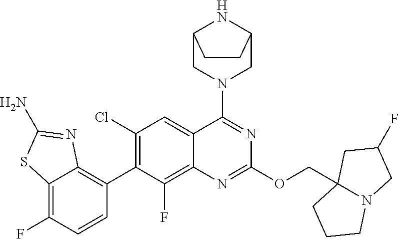 [SMILES: Nc1nc2c(-c3c(Cl)cc4c(N5CC6CCC(C5)N6)nc(OCC56CCCN5CC(F)C6)nc4c3F)ccc(F)c2s1]

### Step 1: tert-butyl-3-(7-(2-((tert-butoxycarbonyl)amino)-7-fluorobenzo[d]thiazol-4-yl)-6-chloro-8-fluoro-2-((2-fluorotetrahydro-1H-pyrrolizin-7a(5H)-yl)methoxy)quinazolin-4-yl)-3,8-diazabicyclo[3.2.1]octane-8-carboxylate

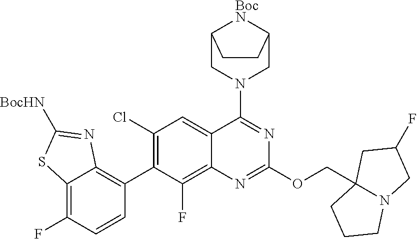 [SMILES: CC(C)(C)OC(=O)Nc1nc2c(-c3c(Cl)cc4c(N5CC6CCC(C5)N6C(=O)OC(C)(C)C)nc(OCC56CCCN5CC(F)C6)nc4c3F)ccc(F)c2s1]

To a solution of (2-fluorotetrahydro-1H-pyrrolizin-7a(5H)-yl)methanol (47.7 mg, 0.3 mmol) in THF (5 mL) was added sodium hydride (11.5 mg, 0.3 mmol) at 0° C. The resulting mixture was stirred at 0° C. for 1 h. Then tert-butyl-3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (67.6 mg, 0.1 mmol) was added to the reaction mixture and stirred for 2 hrs at room temperature. After completion, the reaction mixture was quenched with water (20 mL), extracted with DCM (50 mL×3), and the organic layers was dried over anhydrous Na₂SO₄, filtered and the filtrate was concentrated under reduced pressure to give the crude title product (100 mg). MS (ESI, m/e) [M+1]⁺816.3.

### Step 2: 4-(4-(3,8-diazabicyclo[3.2.1]octan-3-yl)-6-chloro-8-fluoro-2-((2-fluorotetrahydro-1H-pyrrolizin-7a(5H)-yl)methoxy)quinazolin-7-yl)-7-fluorobenzo[d]thiazol-2-amine

To a solution of tert-butyl-3-(7-(2-((tert-butoxycarbonyl)amino)-7-fluorobenzo[d]thiazol-4-yl)-6-chloro-8-fluoro-2-((2-fluorotetrahydro-1H-pyrrolizin-7a(5H)-yl)methoxy)quinazolin-4-yl)-3,8-diazabicyclo[3.2.1]octane-8-carboxylate (100 mg) in DCM (10 mL) was added TFA (2 mL). The resulting solution was stirred for 4 hrs at room temperature. After completion, the reaction mixture was concentrated to give a residue, which was purified by reverse phase prep-HPLC at room temperature with the following conditions: Column: waters sunfire C18 gel column (19×150 mm), 5 um; Mobile Phase A: 1% aq. HCOOH; Mobile Phase B: MeCN; (Gradient: 17% B increased to 90% B within 11 min, then increased to 90% B within 0.2 min, and 90% B hold 5 min); Flow rate: 17 mL/min; Detector: UV 254 & 280 nm; retention time: 10.1 min. The product containing fractions were collected, rotovaped in vacuo and then lyophilized overnight to give title product (11.6 mg, FA salt). ¹H NMR (500 MHz, DMSO-d₆) δ 8.12 (s, 1H), 7.91 (s, 2H), 7.87 (s, 1H), 7.23-7.20 (m, 1H), 7.09-7.05 (m, 1H), 5.42-5.33 (m, 1H), 4.39 (d, J=10 Hz, 2H), 4.13-4.01 (m, 4H), 3.66 (t, J=15 Hz, 2H), 3.11-3.07 (m, 2H), 2.95-2.85 (m, 2H), 2.13-2.07 (m, 2H), 1.92-1.78 (m, 8H). MS (ESI, m/e) [M+H]⁺ 616.3.

### Example 55: 4-(4-(3-oxa-7,9-diazabicyclo[3.3.1]nonan-9-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

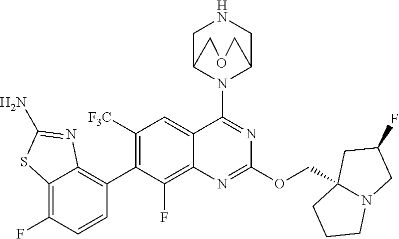 [SMILES: Nc1nc2c(-c3c(C(F)(F)F)cc4c(N5C6CNCC5COC6)nc(OC[C@@]56CCCN5C[C@H](F)C6)nc4c3F)ccc(F)c2s1]

Synthetic Route

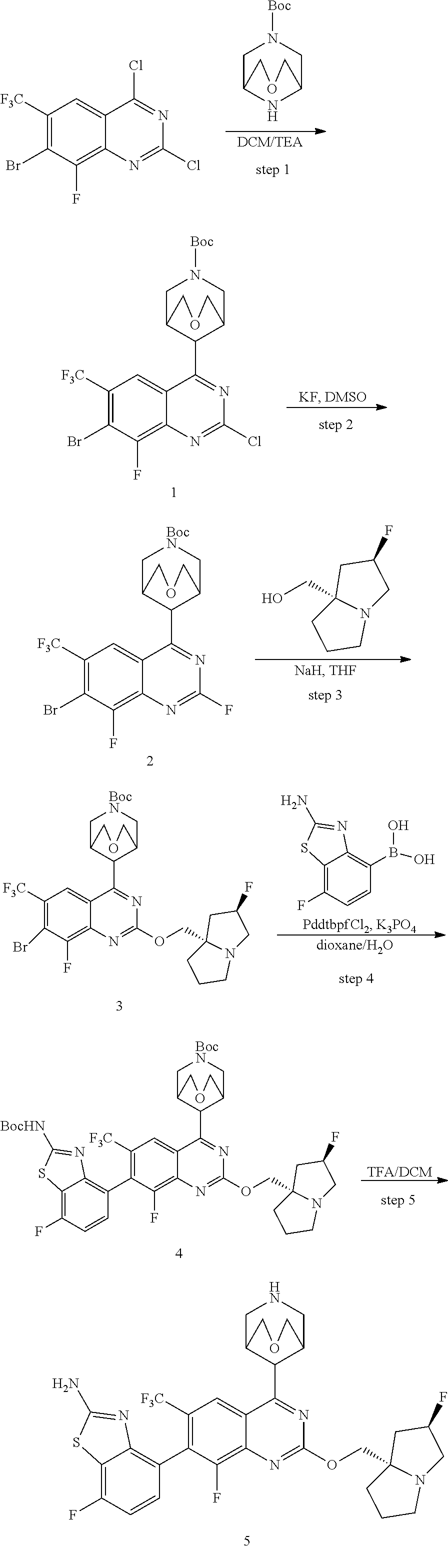

### Step 1: tert-butyl 9-(7-bromo-2-chloro-8-fluoro-6-(trifluoromethyl)quinazolin-4-yl)-3-oxa-7,9-diazabicyclo[3.3.1]nonane-7-carboxylate

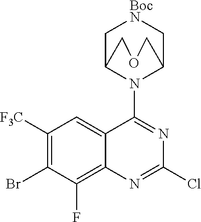 [SMILES: CC(C)(C)OC(=O)N1CC2COCC(C1)N2c1nc(Cl)nc2c(F)c(Br)c(C(F)(F)F)cc12]

To a solution of 7-bromo-2,4-dichloro-8-fluoro-6-(trifluoromethyl)quinazoline (300 mg, 0.83 mmol), tert-butyl 3-oxa-7,9-diazabicyclo[3.3.1]nonane-7-carboxylate (189.1 mg, 0.83 mmol) in DCM (5 mL) was added TEA (167.44 mg, 1.66 mmol) at room temperature. The resulting mixture was stirred at room temperature for 1 hrs. After completion. The reaction mixture was diluted with EA (50 mL), washed with saturated NaCl (15 mL×3). The organic layer was dried over anhydrous Na₂SO₄, filtered and the filtrate was concentrated to give the residue. The residue was purified by Prep-TLC (PE:EA=3:1) to give the title product (300 mg, 0.54 mmol). MS (ESI, m/e) [M+1]⁺555.0, 557.0.

### Step 2: tert-butyl 9-(7-bromo-2,8-difluoro-6-(trifluoromethyl)quinazolin-4-yl)-3-oxa-7,9-diazabicyclo[3.3.1]nonane-7-carboxylate

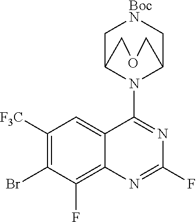 [SMILES: CC(C)(C)OC(=O)N1CC2COCC(C1)N2c1nc(F)nc2c(F)c(Br)c(C(F)(F)F)cc12]

To a solution of tert-butyl 9-(7-bromo-2-chloro-8-fluoro-6-(trifluoromethyl)quinazolin-4-yl)-3-oxa-7,9-diazabicyclo[3.3.1]nonane-7-carboxylate (300 mg, 0.54 mmol) in DMSO (5 mL) was added KF (314.6 mg, 5.4 mmol) at room temperature. The resulting solution was stirred for 16 hrs at 120° C. After completion. The reaction mixture was diluted with EA (50 mL), washed with saturated NaCl (10 mL×3). The organic layer was dried over anhydrous Na₂SO₄, filtered and the filtrate was concentrated to give a residue. the residue was purified by silica gel column chromatography, eluted with 0-30% EA in PE to give the title product (170 mg, 0.32 mmol). MS (ESI, m/e) [M+1]⁺539.1, 541.1.

### Step 3: tert-butyl 9-(7-bromo-8-fluoro-2-(((2R,7aS)-2-fluorotetrahydro-1H-pyrrolizin-7a(5H)-yl)methoxy)-6-(trifluoromethyl)quinazolin-4-yl)-3-oxa-7,9-diazabicyclo[3.3.1]nonane-7-carboxylate

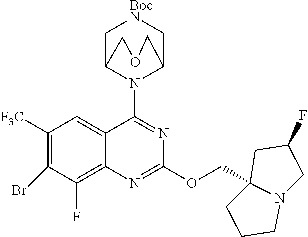 [SMILES: CC(C)(C)OC(=O)N1CC2COCC(C1)N2c1nc(OC[C@@]23CCCN2C[C@H](F)C3)nc2c(F)c(Br)c(C(F)(F)F)cc12]

To a solution of ((2R,7aS)-2-fluorotetrahydro-1H-pyrrolizin-7a(5H)-yl)methanol (101.8 mg, 0.64 mmol) in THF (5 mL) was added sodium hydride (101.8 mg, 0.64 mmol) at room temperature. The resulting mixture was stirred at room temperature for 1 hr, Then tert-butyl 9-(7-bromo-2,8-difluoro-6-(trifluoromethyl)quinazolin-4-yl)-3-oxa-7,9-diazabicyclo[3.3.1]nonane-7-carboxylate (170 mg, 0.32 mmol) was added to reaction mixture and stirred for 2 hrs at room temperature. After completion. The reaction mixture was quenched with water (10 mL), extracted with EA (30 mL×3). The combined organic layer was dried over anhydrous Na₂SO₄, filtered and the filtrate was concentrated to give a residue. The residue was purified by silica gel column chromatography, eluted with 0-80% EA in PE to give the title product (70 mg, 0.1 mmol). MS (ESI, m/e) [M+1]⁺678.2, 679.2.

### Step 4: tert-butyl 9-(7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3-oxa-7,9-diazabicyclo[3.3.1]nonane-7-carboxylate

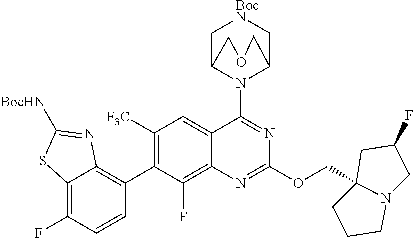 [SMILES: CC(C)(C)OC(=O)Nc1nc2c(-c3c(C(F)(F)F)cc4c(N5C6COCC5CN(C(=O)OC(C)(C)C)C6)nc(OC[C@@]56CCCN5C[C@H](F)C6)nc4c3F)ccc(F)c2s1]

To a solution of tert-butyl 9-(7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3-oxa-7,9-diazabicyclo[3.3.1]nonane-7-carboxylate (35 mg, 0.05 mmol), (2-amino-7-fluorobenzo[d]thiazol-4-yl)boronic acid (46.45 mg, 0.15 mmol) and Pd(dtbpf)Cl₂ (3.25 mg, 0.005 mmol) in dioxane (5 mL) and H₂O (1 mL) was added K₃PO₄ (63.6 mg, 0.15 mmol) at room temperature, The resulting mixture was stirred at 100° C. for 16 hrs under N₂ atmosphere. After completion. The reaction mixture was diluted with EA (30 mL), washed with saturated NaCl (250 mL×3). The organic layer was dried over anhydrous Na₂SO₄, filtered and the filtrate was concentrated to give the residue. The residue was purified by Prep-TLC (pure EA) to give the title product (15 mg, 0.017 mmol), MS (ESI, m/e) [M+1]⁺866.3.

### Step 5: 4-(4-(3-oxa-7,9-diazabicyclo[3.3.1]nonan-9-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

To a solution of tert-butyl 9-(7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3-oxa-7,9-diazabicyclo[3.3.1]nonane-7-carboxylate (15 mg, 0.017 mmol) in DCM (5 mL) was added TFA (2 mL) at room temperature. The resulting mixture was stirred at room temperature for 3 hrs. After completion. The reaction mixture was concentrated to give the residue. The residue was purified by Prep-HPLC to give the title product (1.77 mg). ¹H NMR (500 MHz, CD₃OD) δ 8.02 (s, 1H), 7.20-7.17 (m, 1H), 7.0-6.96 (m, 1H), 5.51-5.38 (m, 1H), 4.71 (s, 2H), 4.54-4.47 (m, 2H), 4.30-4.27 (m, 4H), 3.79-3.57 (m, 8H), 2.53-2.41 (m, 2H), 2.29-2.18 (m, 3H), 2.04-2.02 (m, 1H) MS (ESI, m/e) [M+1]⁺666.4

### Intermediate 156: benzyl 4-(7-(2-amino-7-fluorobenzo[d]thiazol-4-yl)-6-chloro-8-fluoro-2-(3-(piperazin-1-yl)propoxy)quinazolin-4-yl)piperazine-1-carboxylate

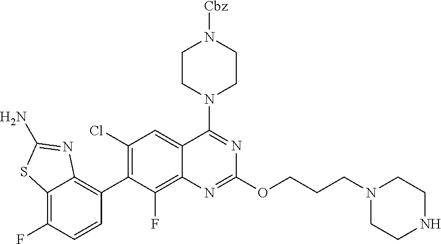 [SMILES: Nc1nc2c(-c3c(Cl)cc4c(N5CCN(C(=O)OCc6ccccc6)CC5)nc(OCCCN5CCNCC5)nc4c3F)ccc(F)c2s1]

Synthetic Route

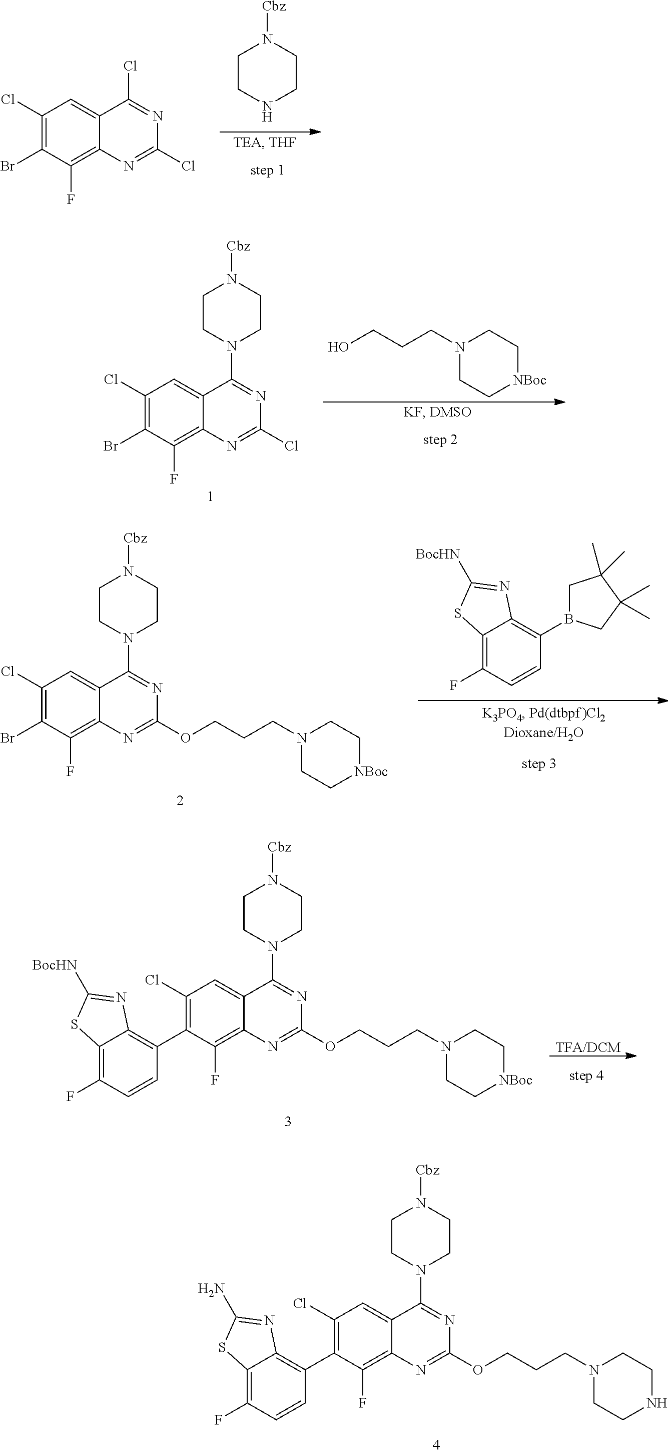

### Step 1: benzyl 4-(7-bromo-2,6-dichloro-8-fluoroquinazolin-4-yl)piperazine-1-carboxylate

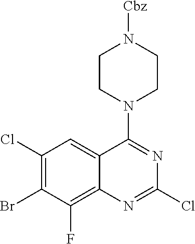 [SMILES: O=C(OCc1ccccc1)N1CCN(c2nc(Cl)nc3c(F)c(Br)c(Cl)cc23)CC1]

To a solution of 7-bromo-2,4,6-trichloro-8-fluoroquinazoline (32.8 g, 100 mmol), benzyl piperazine-1-carboxylate (22.1 g, 100 mmol) in THF (500 mL) was added TEA (267 mL, 200 mmol) at room temperature. The resulting mixture was stirred at room temperature for 1 hr. After completion. The reaction mixture was poured to water, filtered to give the title product (43.0 g, 83.8 mmol). MS (ESI, m/e) [M+1]⁺513.0, 515.0.

### Step 2: benzyl 4-(7-bromo-2-(3-(4-(tert-butoxycarbonyl)piperazin-1-yl)propoxy)-6-chloro-8-fluoroquinazolin-4-yl)piperazine-1-carboxylate

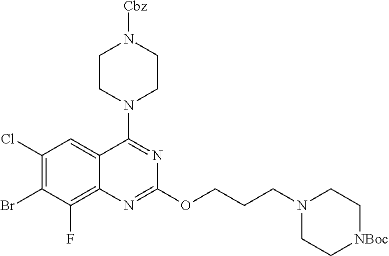 [SMILES: CC(C)(C)OC(=O)N1CCN(CCCOc2nc(N3CCN(C(=O)OCc4ccccc4)CC3)c3cc(Cl)c(Br)c(F)c3n2)CC1]

To a solution of benzyl 4-(7-bromo-2,6-dichloro-8-fluoroquinazolin-4-yl)piperazine-1-carboxylate (43.0 g, 83.8 mmol), tert-butyl 4-(3-hydroxypropyl)piperazine-1-carboxylate (26.3 g, 117.3 mmol) in DMSO (500 mL) was added KF (14.6 g, 251.2 mmol) at room temperature. The resulting solution was stirred for 16 hrs at 120° C. After completion. The reaction mixture was diluted with EA (1 L), washed with saturated NaCl (250 mL×3). The organic layer was dried over anhydrous Na₂SO₄, filtered and the filtrate was concentrated to give the residue. The residue was purified by silica gel column chromatography, eluted with 0-30% EA in PE to give the title product (24.0 g, 33.3 mmol). MS (ESI, m/e) [M+1]⁺721.2, 723.2.

### Step 3: benzyl 4-(7-(2-((tert-butoxycarbonyl)amino)-7-fluorobenzo[d]thiazol-4-yl)-2-(3-(4-(tert-butoxycarbonyl)piperazin-1-yl)propoxy)-6-chloro-8-fluoroquinazolin-4-yl)piperazine-1-carboxylate

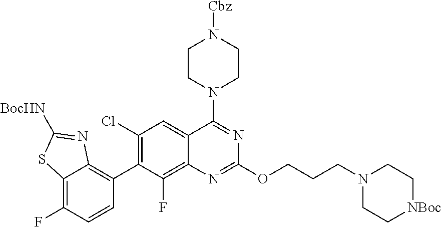 [SMILES: CC(C)(C)OC(=O)Nc1nc2c(-c3c(Cl)cc4c(N5CCN(C(=O)OCc6ccccc6)CC5)nc(OCCCN5CCN(C(=O)OC(C)(C)C)CC5)nc4c3F)ccc(F)c2s1]

To a solution of benzyl 4-(7-bromo-2-(3-(4-(tert-butoxycarbonyl)piperazin-1-yl)propoxy)-6-chloro-8-fluoroquinazolin-4-yl)piperazine-1-carboxylate (24.0 g, 33.3 mmol), tert-butyl (7-fluoro-4-(4,4,5,5-tetramethyl-1,3,2-dioxaborolan-2-yl)benzo[d]thiazol-2-yl)carbamate (15.77 g, 40 mmol) and Pd(dtbpf)Cl₂ (2.17 g, 3.33 mmol) in dioxane (500 mL) and H₂O (100 mL) was added K₃PO₄ (21.2 g, 100 mmol) at room temperature. The resulting mixture was stirred at 100° C. for 16 hrs, After completion, The reaction mixture was diluted with EA (1.5 L), washed with saturated NaCl (250 mL×3). The organic layer was dried over anhydrous Na₂SO₄, filtered and the filtrate was concentrated to give the residue. The residue was purified by silica gel column chromatography, eluted with 0-80% EA in PE to give the title product (22.0 g, 24.2 mmol). MS (ESI, m/e) [M+1]⁺909.3.

### Step 4: benzyl 4-(7-(2-amino-7-fluorobenzo[d]thiazol-4-yl)-6-chloro-8-fluoro-2-(3-(piperazin-1-yl)propoxy)quinazolin-4-yl)piperazine-1-carboxylate

To a solution of benzyl 4-(7-(2-((tert-butoxycarbonyl)amino)-7-fluorobenzo[d]thiazol-4-yl)-2-(3-(4-(tert-butoxycarbonyl)piperazin-1-yl)propoxy)-6-chloro-8-fluoroquinazolin-4-yl)piperazine-1-carboxylate (22.0 g, 24.2 mmol) in DCM (100 mL) was added TFA (50 mL) at room temperature. The resulting solution was stirred for 4 hrs at room temperature. After completion. The reaction mixture was concentrated to give the residue. Then NH₃·H₂O (50 mL) was added to the residue, filtered to give the title product (15.0 g, 21.1 mmol). MS (ESI, m/e) [M+H]⁺ 709.2

### Example 56: N-(2-(2-(3-(3-(4-(3-((7-(2-amino-7-fluorobenzo[d]thiazol-4-yl)-6-chloro-8-fluoro-4-(piperazin-1-yl)quinazolin-2-yl)oxy)propyl)piperazin-1-yl)propoxy)propanamido)ethoxy)ethyl)-5-((3 aS, 4S,6aR)-2-oxohexahydro-1H-thieno[3,4-d]imidazol-4-yl)pentanamide

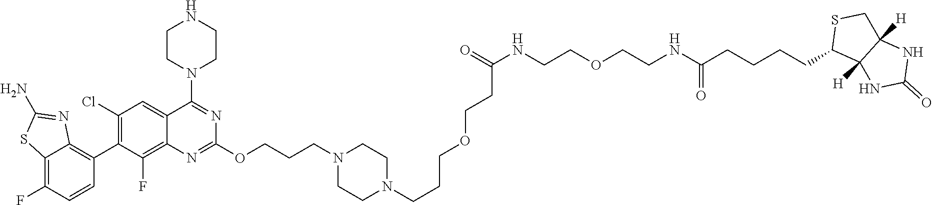 [SMILES: Nc1nc2c(-c3c(Cl)cc4c(N5CCNCC5)nc(OCCCN5CCN(CCCOCCC(=O)NCCOCCNC(=O)CCCC[C@@H]6SC[C@@H]7NC(=O)N[C@@H]76)CC5)nc4c3F)ccc(F)c2s1]

Synthetic Route

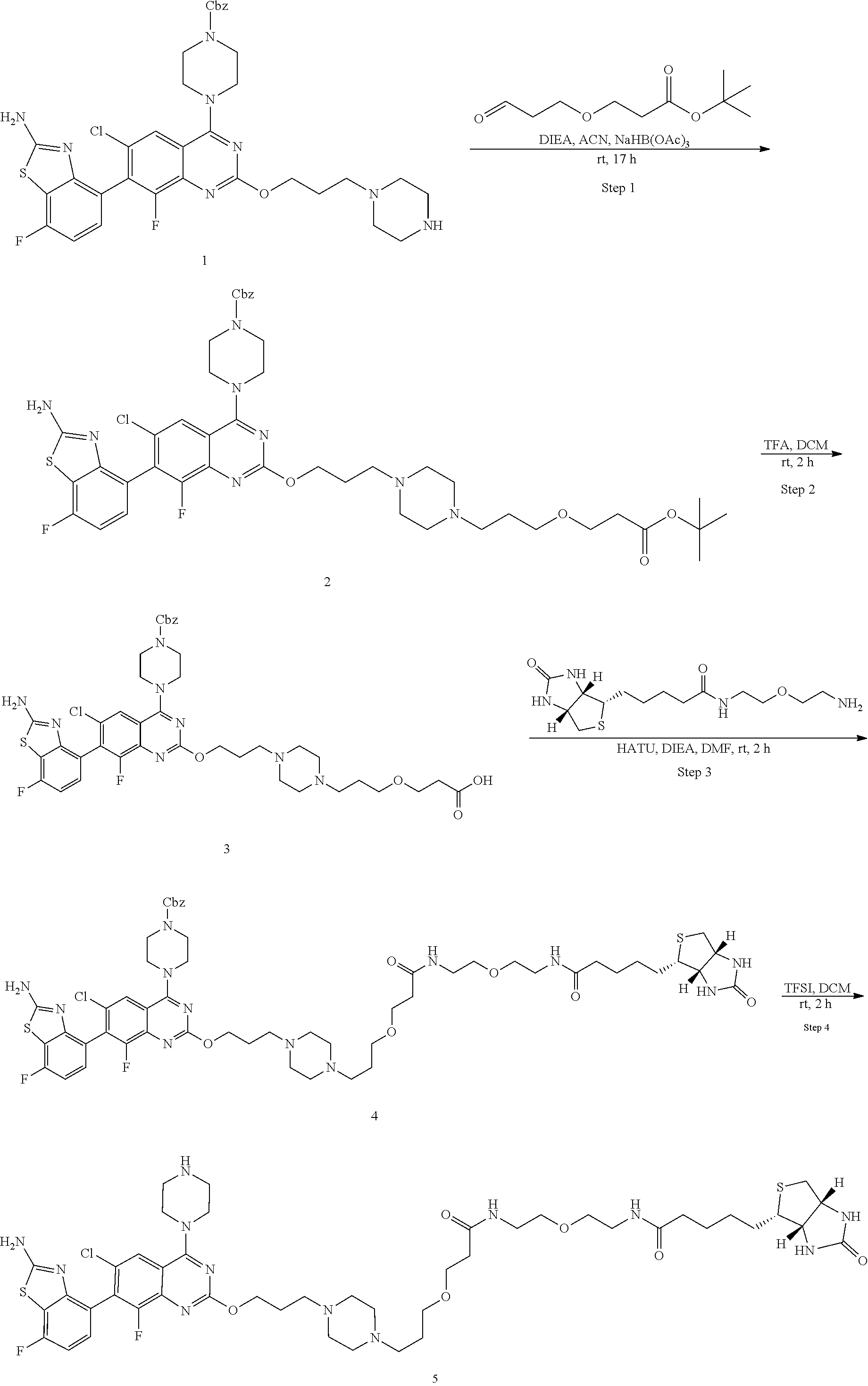

### Step 1: benzyl 4-(7-(2-amino-7-fluorobenzo[d]thiazol-4-yl)-2-(3-(4-(3-(3-(tert-butoxy)-3-oxopropoxy)propyl)piperazin-1-yl)propoxy)-6-chloro-8-fluoroquinazolin-4-yl)piperazine-1-carboxylate

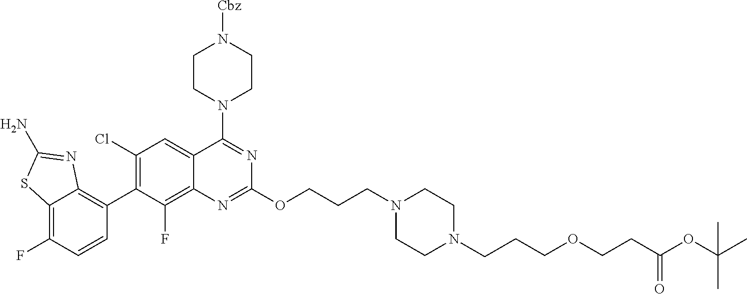 [SMILES: CC(C)(C)OC(=O)CCOCCCN1CCN(CCCOc2nc(N3CCN(C(=O)OCc4ccccc4)CC3)c3cc(Cl)c(-c4ccc(F)c5sc(N)nc45)c(F)c3n2)CC1]

To a mixture of benzyl 4-(7-(2-amino-7-fluorobenzo[d]thiazol-4-yl)-6-chloro-8-fluoro-2-(3-(piperazin-1-yl)propoxy)quinazolin-4-yl)piperazine-1-carboxylate (1 g, 1.4 mmol) in CH₃CN (100 mL) was added DIPEA (300 mg, 2.3 mmol), and NaHB(AcO)₃ (600 mg, 2.8 mmol), stirred at room temperature for 17 hours. The resulting mixture was filtered with celite and the filtrate was concentrated and purified by combi-flash (DCM/MeOH/NH₃H₂O=10/1/0.1) to give the title product (800 mg, 64%). MS (ESI, m/e) [M+H]⁺ 895.7

### Step 2: 3-(3-(4-(3-((7-(2-amino-7-fluorobenzo[d]thiazol-4-yl)-4-(4-((benzyloxy)carbonyl)piperazin-1-yl)-6-chloro-8-fluoroquinazolin-2-yl)oxy)propyl)piperazin-1-yl)propoxy)propanoic acid

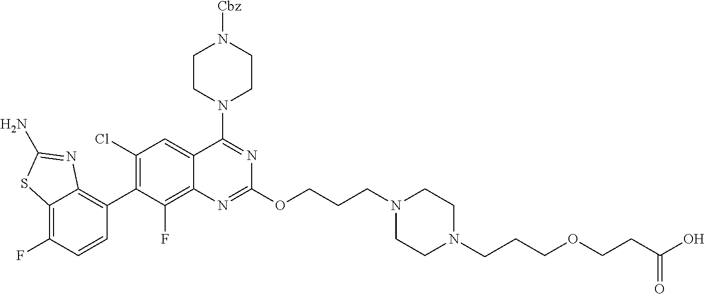 [SMILES: Nc1nc2c(-c3c(Cl)cc4c(N5CCN(C(=O)OCc6ccccc6)CC5)nc(OCCCN5CCN(CCCOCCC(=O)O)CC5)nc4c3F)ccc(F)c2s1]

To a solution of benzyl 4-(7-(2-amino-7-fluorobenzo[d]thiazol-4-yl)-2-(3-(4-(3-(3-(tert-butoxy)-3-oxopropoxy)propyl)piperazin-1-yl)propoxy)-6-chloro-8-fluoroquinazolin-4-yl)piperazine-1-carboxylate (800 mg, 0.9 mmol) in DCM (50 mL) was added TFA (10 mL) and stirred at room temperature for 2 hours. The resulting solution was concentrated to give the title product (1 g, crude) as thick brown oil used directly in next step. MS (ESI, m/e) [M+H]⁺ 839.6

### Step 3: benzyl 4-(7-(2-amino-7-fluorobenzo[d]thiazol-4-yl)-6-chloro-2-(3-(4-(7,15-dioxo-19-((3aS,4S,6aR)-2-oxohexahydro-1H-thieno[3,4-d]imidazol-4-yl)-4,11-dioxa-8,14-diazanonadecyl)piperazin-1-yl)propoxy)-8-fluoroquinazolin-4-yl)piperazine-1-carboxylate

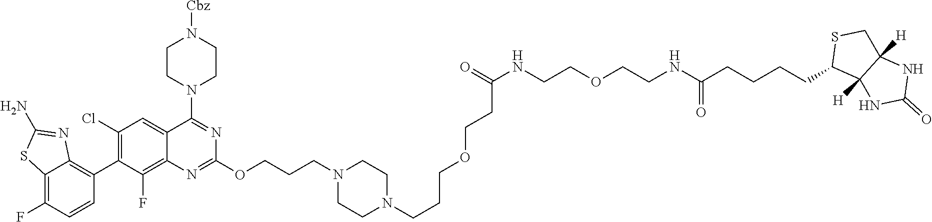 [SMILES: Nc1nc2c(-c3c(Cl)cc4c(N5CCN(C(=O)OCc6ccccc6)CC5)nc(OCCCN5CCN(CCCOCCC(=O)NCCOCCNC(=O)CCCC[C@@H]6SC[C@@H]7NC(=O)N[C@@H]76)CC5)nc4c3F)ccc(F)c2s1]

To a solution of 3-(3-(4-(3-((7-(2-amino-7-fluorobenzo[d]thiazol-4-yl)-4-(4-((benzyloxy)carbonyl)piperazin-1-yl)-6-chloro-8-fluoroquinazolin-2-yl)oxy)propyl)piperazin-1-yl)propoxy)propanoic acid (200 mg, 0.24 mmol), N-(2-(2-aminoethoxy)ethyl)-5-((3aS,4S,6aR)-2-oxohexahydro-1H-thieno[3,4-d]imidazol-4-yl)pentanamide (80 mg, 0.24 mmol) and HATU (136 mg, 0.36 mmol) in DMF (15 mL) was added DIPEA (92 mg, 0.72 mmol) and stirred at room temperature for 2 hours. The resulting solution was concentrated and purified by Pre-TLC (DCM/MeOH/NH₃H₂O=10/1/0.1) to give the title product (100 mg, 36%) as brown solid. MS (ESI, m/e) [M/2+H]⁺ 576.5

### Step 4: N-(2-(2-(3-(3-(4-(3-((7-(2-amino-7-fluorobenzo[d]thiazol-4-yl)-6-chloro-8-fluoro-4-(piperazin-1-yl)quinazolin-2-yl)oxy)propyl)piperazin-1-yl)propoxy)propanamido)ethoxy)ethyl)-5-((3aS,4S,6aR)-2-oxohexahydro-1H-thieno[3,4-d]imidazol-4-yl)pentanamide

A solution of benzyl 4-(7-(2-amino-7-fluorobenzo[d]thiazol-4-yl)-6-chloro-2-(3-(4-(7,15-dioxo-19-((3aS,4S,6aR)-2-oxohexahydro-1H-thieno[3,4-d]imidazol-4-yl)-4,11-dioxa-8,14-diazanonadecyl)piperazin-1-yl)propoxy)-8-fluoroquinazolin-4-yl)piperazine-1-carboxylate (90 mg, 0.08 mmol) in DCM (20 mL) was added TMSI (200 mg, 1 mmol), stirred at 25° C. for 2 hours. The resulting solution was concentrated and purified by Prep-HPLC to give the title product (44 mg, 54%, FA salt) as white solid. ¹H NMR (500 MHz, CD₃OD) δ 8.36 (s, 1H), 7.93 (s, 1H), 7.24-7.21 (m, 1H), 7.02-6.99 (m, 1H), 4.59-4.51 (m, 2H), 4.49-4.46 (m, 1H), 4.30-4.27 (m, 1H), 4.07-4.05 (m, 4H), 3.69-3.67 (m, 2H), 3.59-3.56 (m, 2H), 3.53-3.50 (m, 4H), 3.47-3.45 (m, 4H), 3.38-3.33 (m, 4H), 3.20-3.06 (m, 7H), 2.93-2.78 (m, 7H), 2.70-2.66 (m, 1H), 2.48-2.45 (m, 2H), 2.22-2.19 (m, 2H), 2.12-2.07 (m, 2H), 1.96-1.91 (m, 2H), 1.75-1.53 (m, 4H), 1.45-1.39 (m, 2H). MS (ESI, m/e) [M+H]⁺ 1017.7.

### Intermediate 157: methyl 3-(3-((S)-2-(((7-(8-chloronaphthalen-1-yl)-4-((S)-3-(cyanomethyl)piperazin-1-yl)-5,6,7,8-tetrahydropyrido[3,4-d]pyrimidin-2-yl)oxy)methyl)pyrrolidin-1-yl)propoxy)propanoate

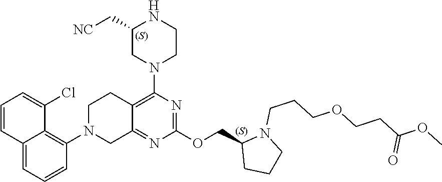

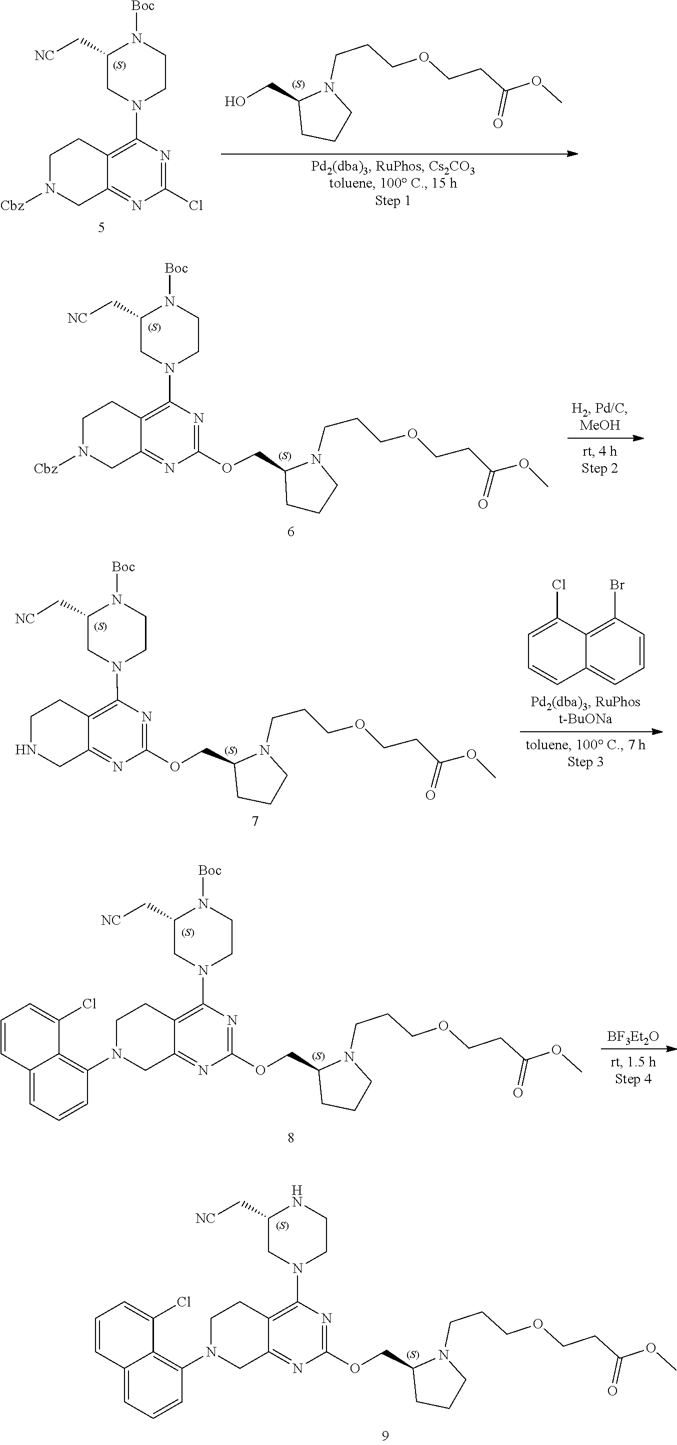

### Step 1: benzyl 4-((S)-4-(tert-butoxycarbonyl)-3-(cyanomethyl)piperazin-1-yl)-2-(((S)-1-(3-(3-methoxy-3-oxopropoxy)propyl)pyrrolidin-2-yl)methoxy)-5,8-dihydropyrido[3,4-d]pyrimidine-7(6H)-carboxylate

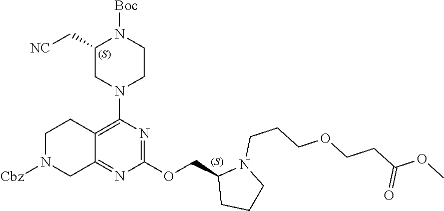

A mixture of benzyl (S)-4-(4-(tert-butoxycarbonyl)-3-(cyanomethyl)piperazin-1-yl)-2-chloro-5,8-dihydropyrido[3,4-d]pyrimidine-7(6H)-carboxylate (2.63 g, 5 mmol), methyl (S)-3-(3-(2-(hydroxymethyl)pyrrolidin-1-yl)propoxy)propanoate (1.84 g, 7.5 mmol), Pd₂(dba)₃ (916 mg, 1 mmol), RuPhOS (916 mg, 2 mmol) and Cs₂CO₃ (4.89 g, 15 mmol) was added toluene (50 mL), degassed with N₂ gas three times, stirred at 100° C. for 14 hours. The cooled resulting mixture was filtered and the filtrate was concentrated and purified by combi-flash (DCM/MeOH=10/1) to give the title product (2.2 g, 60%) as brown foam. MS (ESI, m/e) [M+H]⁺ 736.6.

### Step 2: tert-butyl (S)-2-(cyanomethyl)-4-(2-(((S)-1-(3-(3-methoxy-3-oxopropoxy)propyl)pyrrolidin-2-yl)methoxy)-5,6,7,8-tetrahydropyrido[3,4-d]pyrimidin-4-yl)piperazine-1-carboxylate

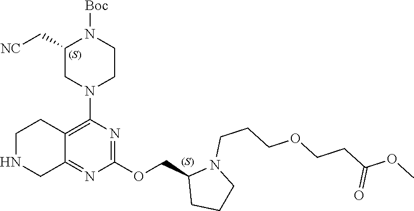

A solution of benzyl 4-((S)-4-(tert-butoxycarbonyl)-3-(cyanomethyl)piperazin-1-yl)-2-(((S)-1-(3-(3-methoxy-3-oxopropoxy)propyl)pyrrolidin-2-yl)methoxy)-5,8-dihydropyrido[3,4-d]pyrimidine-7(6H)-carboxylate (2.2 g, 3 mmol) in MeOH (70 mL) was added Pd/C (10% wet, 1 g) and degassed with H₂ gas three times, stirred at 25° C. for 4 hours under H₂ balloon. The resulting mixture was filtered and the filtrate was concentrated to give the title product (1.68 g, 93%) as brown foam. MS (ESI, m/e) [M+H]⁺ 602.6.

### Step 3: tert-butyl (S)-4-(7-(8-chloronaphthalen-1-yl)-2-(((S)-1-(3-(3-methoxy-3-oxopropoxy)propyl)pyrrolidin-2-yl)methoxy)-5,6,7,8-tetrahydropyrido[3,4-d]pyrimidin-4-yl)-2-(cyanomethyl)piperazine-1-carboxylate

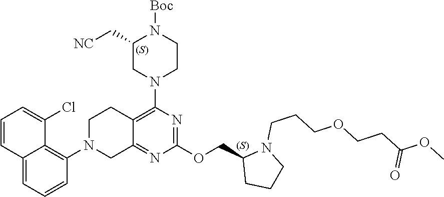

A mixture of tert-butyl (S)-2-(cyanomethyl)-4-(2-(((S)-1-(3-(3-methoxy-3-oxopropoxy)propyl)pyrrolidin-2-yl)methoxy)-5,6,7,8-tetrahydropyrido[3,4-d]pyrimidin-4-yl)piperazine-1-carboxylate (200 mg, 0.33 mmol), 1-bromo-8-chloronaphthalene (400 mg, 1.66 mmol), Pd₂(dba)₃ (60 mg, 0.066 mmol), RuPhOS (62 mg, 0.132 mmol) and t-BuONa (64 g, 0.67 mmol) was added toluene (20 mL), degassed with N₂ gas three times, stirred at 100° C. for 5 hours. The cooled resulting mixture was filtered and the filtrate was concentrated and purified by combi-flash (DCM/MeOH=10/1) to give the title product (153 mg, 61%) as brown foam. MS (ESI, m/e) [M+H]⁺ 762.5.

### Step 4: methyl 3-(3-((S)-2-(((7-(8-chloronaphthalen-1-yl)-4-((S)-3-(cyanomethyl)piperazin-1-yl)-5,6,7,8-tetrahydropyrido[3,4-d]pyrimidin-2-yl)oxy)methyl)pyrrolidin-1-yl)propoxy)propanoate

A solution of tert-butyl (S)-4-(7-(8-chloronaphthalen-1-yl)-2-(((S)-1-(3-(3-methoxy-3-oxopropoxy)propyl)pyrrolidin-2-yl)methoxy)-5,6,7,8-tetrahydropyrido[3,4-d]pyrimidin-4-yl)-2-(cyanomethyl)piperazine-1-carboxylate (153 mg, 0.2 mmol) in DCM (20 mL) was added BF₃Et₂O (100 mg, 0.7 mmol), stirred at 25° C. for 1 hour. The resulting solution was concentrated to give the title product (275 mg, crude) as brown foam. MS (ESI, m/e) [M+H]⁺ 662.4.

### Example 57: N-(2-(2-(3-(3-((S)-2-(((7-(8-chloronaphthalen-1-yl)-4-((S)-3-(cyanomethyl)-4-(methylsulfonyl)piperazin-1-yl)-5,6,7,8-tetrahydropyrido[3,4-d]pyrimidin-2-yl)oxy)methyl)pyrrolidin-1-yl)propoxy)propanamido)ethoxy)ethyl)-5-((3aS,4S,6aR)-2-oxohexahydro-1H-thieno[3,4-d]imidazol-4-yl)pentanamide

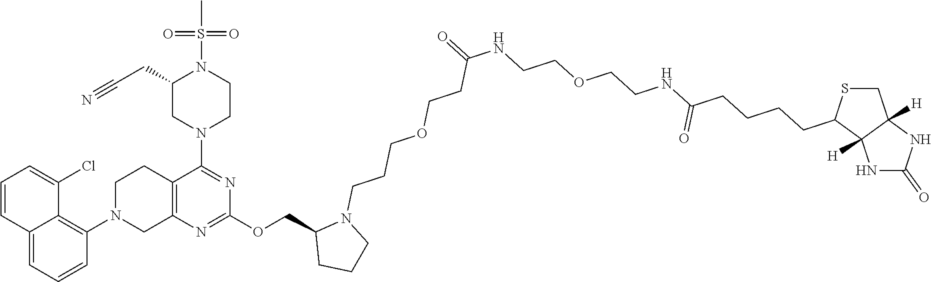 [SMILES: CS(=O)(=O)N1CCN(c2nc(OC[C@@H]3CCCN3CCCOCCC(=O)NCCOCCNC(=O)CCCCC3SC[C@@H]4NC(=O)N[C@H]34)nc3c2CCN(c2cccc4cccc(Cl)c24)C3)C[C@@H]1CC#N]

Synthetic Route

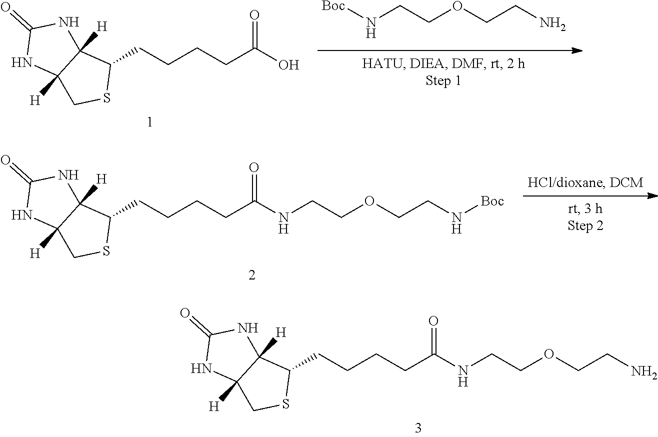 [SMILES: O=C(O)CCCC[C@@H]1SC[C@@H]2NC(=O)N[C@@H]21 ; CC(C)(C)OC(=O)NCCOCCN ; CC(C)(C)OC(=O)NCCOCCNC(=O)CCCC[C@@H]1SC[C@@H]2NC(=O)N[C@@H]21 ; NCCOCCNC(=O)CCCC[C@@H]1SC[C@@H]2NC(=O)N[C@@H]21]

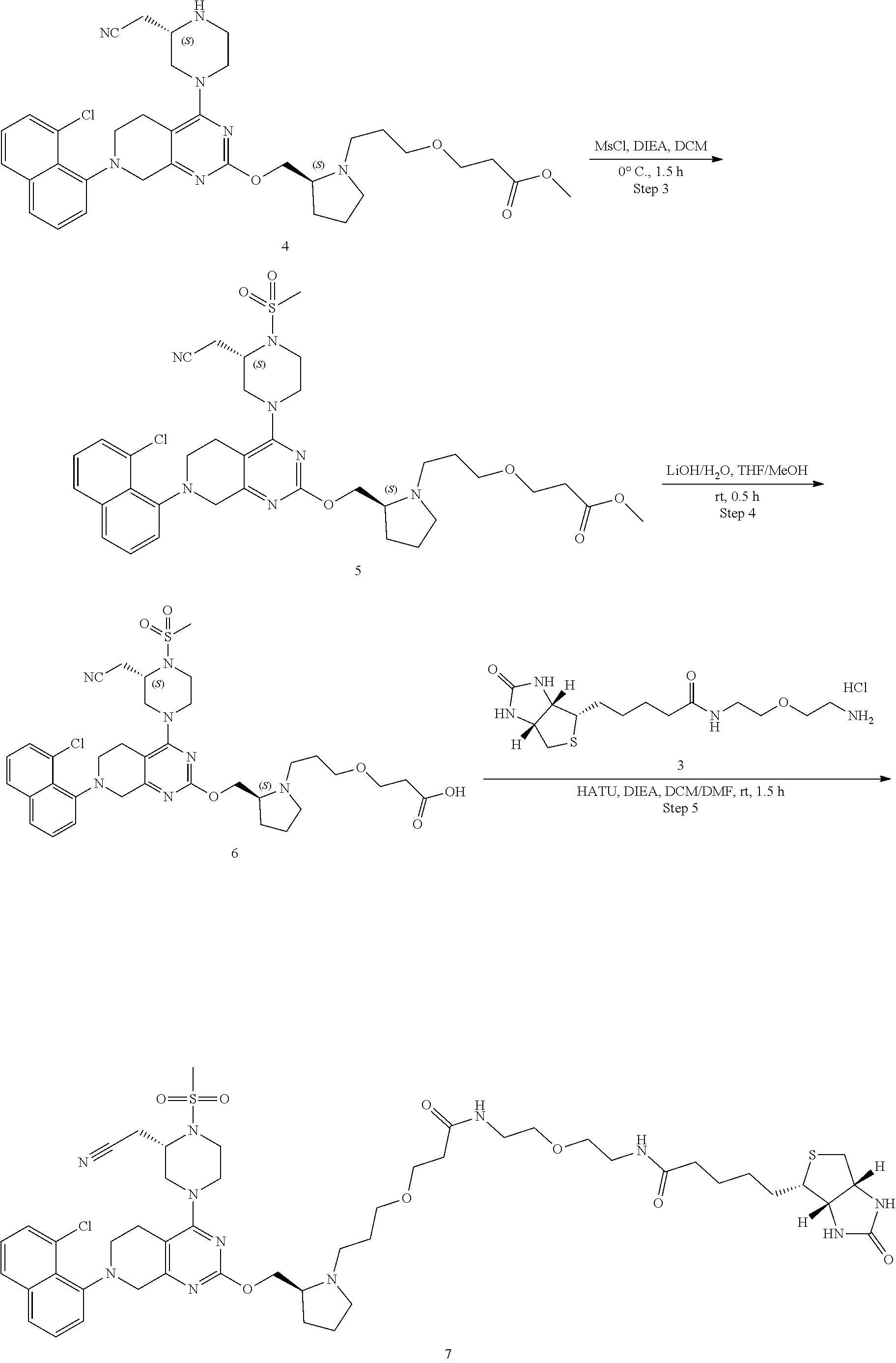

### Step 1: tert-butyl (2-(2-(5-((3aS,4S,6aR)-2-oxohexahydro-1H-thieno[3,4-d]imidazol-4-yl)pentanamido)ethoxy)ethyl)carbamate

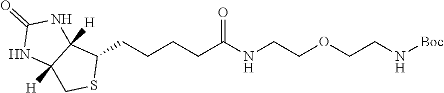 [SMILES: CC(C)(C)OC(=O)NCCOCCNC(=O)CCCC[C@@H]1SC[C@@H]2NC(=O)N[C@@H]21]

To a solution of 5-((3aS,4S,6aR)-2-oxohexahydro-1H-thieno[3,4-d]imidazol-4-yl)pentanoic acid (244 mg, 1 mmol) in DMF (20 mL) was added HATU (380 mg, 1.2 mmol), DIPEA (380 mg, 3 mmol) and tert-butyl (2-(2-aminoethoxy)ethyl)carbamate (204 mg, 1 mmol), stirred at room temperature for 2 hours. The resulting solution was concentrated and purified by combi-flash (DCM/MeOH/NH₃H₂O=10/1/0.1) to give the title product (430 mg, 100%). MS (ESI, m/e) [M+14]⁺431.3.

### Step 2: N-(2-(2-aminoethoxy)ethyl)-5-43aS,4S,6aR)-2-oxohexahydro-1H-thieno[3,4-d]imidazol-4-yl)pentanamide

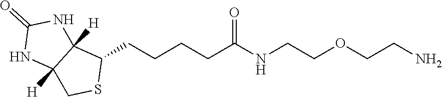 [SMILES: NCCOCCNC(=O)CCCC[C@@H]1SC[C@@H]2NC(=O)N[C@@H]21]

To a solution of tert-butyl (2-(2-(5-((3aS,4S,6aR)-2-oxohexahydro-1H-thieno[3,4-d]imidazol-4-yl)pentanamido)ethoxy)ethyl)carbamate (100 mg, 0.23 mmol) in DCM (5 mL) was added 4M HCl/dioxane (2.5 mL) and stirred at room temperature for 1 hour. The resulting solution was concentrated to give the title product (100 mg, 30%, HCl salt) as white solid. MS (ESI, m/e) [M+H]⁺331.2.

### Step 3: methyl 3-(3-((S)-2-(((7-(8-chloronaphthalen-1-yl)-4-((S)-3-(cyanomethyl)-4-(methylsulfonyl)piperazin-1-yl)-5,6,7,8-tetrahydropyrido[3,4-d]pyrimidin-2-yl)oxy)methyl)pyrrolidin-1-yl)propoxy)propanoate

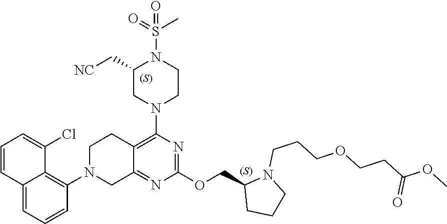

To a solution of methyl 3-(3-((S)-2-(((7-(8-chloronaphthalen-1-yl)-4-((S)-3-(cyanomethyl)piperazin-1-yl)-5,6,7,8-tetrahydropyrido[3,4-d]pyrimidin-2-yl)oxy)methyl)pyrrolidin-1-yl)propoxy)propanoate (290 mg, 0.44 mmol) in DCM was added DIPEA (375 mg, 2.91 mmol) and MsCl (200 mg, 1.75 mmol), stirred at 0° C. for 1.5 hours. The resulting solution was washed with brine (30 mL*2) and dried over Na₂SO₄, the solution was concentrated and purified by Pre-TLC (DCM/MeOH/NH₃H₂O=10/1/0.1) to give the title product (130 mg, 40%). MS (ESI, m/e) [M/2+H]⁺740.4.

### Step 4: 3-(3-((S)-2-(((7-(8-chloronaphthalen-1-yl)-4-((S)-3-(cyanomethyl)-4-(methylsulfonyl)piperazin-1-yl)-5,6,7,8-tetrahydropyrido[3,4-d]pyrimidin-2-yl)oxy)methyl)pyrrolidin-1-yl)propoxy)propanoic acid

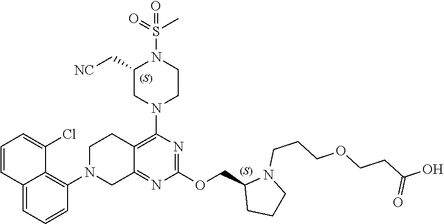

To a solution of methyl 3-(3-((S)-2-(((7-(8-chloronaphthalen-1-yl)-4-((S)-3-(cyanomethyl)-4-(methylsulfonyl)piperazin-1-yl)-5,6,7,8-tetrahydropyrido[3,4-d]pyrimidin-2-yl)oxy)methyl)pyrrolidin-1-yl)propoxy)propanoate (130 mg, 0.18 mmol) in methanol (5 mL) was added THF (5 mL) and LiOH/H₂O (1M, 2.5 mL), stirred at room temperature for 0.5 hour. Then it was neutralized by HCl/H₂O (1M) to pH=5-6, and the solution was evaporated, dissolved in DCM (10 mL) and filtered the solid, the filtrate was concentrated and dried to give the title product (127 mg, 99%). MS (ESI, m/e) [M+H]⁺ 726.4.

### Step 5: N-(2-(2-(3-(3-((S)-2-(((7-(8-chloronaphthalen-1-yl)-4-((S)-3-(cyanomethyl)-4-(methylsulfonyl)piperazin-1-yl)-5,6,7,8-tetrahydropyrido[3,4-d]pyrimidin-2-yl)oxy)methyl)pyrrolidin-1-yl)propoxy)propanamido)ethoxy)ethyl)-5-((3aS,4S,6aR)-2-oxohexahydro-1H-thieno[3,4-d]imidazol-4-yl)pentanamide

To a mixture of 3-(3-((S)-2-(((7-(8-chloronaphthalen-1-yl)-4-((S)-3-(cyanomethyl)-4-(methylsulfonyl)piperazin-1-yl)-5,6,7,8-tetrahydropyrido[3,4-d]pyrimidin-2-yl)oxy)methyl)pyrrolidin-1-yl)propoxy)propanoic acid (100 mg, 0.14 mmol), N-(2-(2-aminoethoxy)ethyl)-5-((3aS,4S,6aR)-2-oxohexahydro-1H-thieno[3,4-d]imidazol-4-yl)pentanamide hydrochloride (46 mg, 0.14 mmol) and HATU (79 mg, 0.21 mmol) was added DCM (10 mL) and DMF (5 mL), then DIPEA (225 mg, 1.74 mmol) was added and stirred at room temperature for 1.5 hours. The resulting solution was washed with water (20 mL each, 3 times) and dried over Na₂SO₄, the solution was concentrated and purified by Pre-HPLC to give the title product (5.92 mg, 4%) as a FA salt. ¹H NMR (500 MHz, CD₃OD) δ 8.35 (bs, 2H), 7.84-7.83 (m, 1H), 7.70-7.68 m, 1H), 7.554-7.48 (m, 2H), 7.40-7.31 (m, 2H), 4.74-4.70 (m, 2H), 4.50-4.39 (m, 2H), 4.37-4.31 (m, 1H), 4.28-4.16 (m, 2H), 4.06-4.04 (m, 1H), 3.96-3.90 (m, 1H), 3.82-3.56 (m, 10H), 3.52-3.42 (m, 5H), 3.38-23.33 (m, 2H), 3.28-3.11 (m, 7H), 3.10-3.04 (m, 4H), 2.96-2.95 (m, 1H), 2.91-2.88 (m, 1H), 2.76-2.65 (m, 2H), 2.49-2.29 (m, 3H), 2.25-2.09 (m, 4H), 2.09-1.92 (m, 4H), 1.77-1.49 (m, 4H), 1.44-1.38 (m, 2H). MS (ESI, m/e) [M/2+H]⁺ 520.0.

### Example 58: 7-fluoro-4-(8-fluoro-2-(((2R,7aS)-2-fluorotetrahydro-1H-pyrrolizin-7a(5H)-yl) methoxy)-4-((1S,4R)-2-((2-methoxyethyl)amino)-7-azabicyclo[2.2.1]heptan-7-yl)-6-(trifluoromethyl) quinazolin-7-yl)benzo[d]thiazol-2-amine

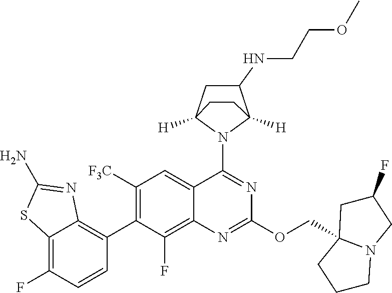 [SMILES: COCCNC1C[C@H]2CC[C@@H]1N2c1nc(OC[C@@]23CCCN2C[C@H](F)C3)nc2c(F)c(-c3ccc(F)c4sc(N)nc34)c(C(F)(F)F)cc12]

Synthetic Route

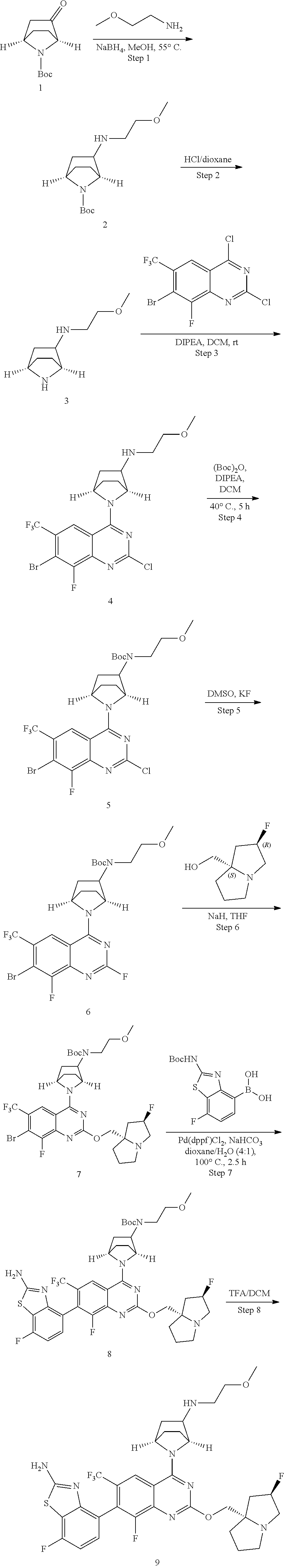

### Step 1: tert-butyl (1S,4R)-2-((2-methoxyethyl) amino)-7-azabicyclo [2.2.1] heptane-7-carboxylate

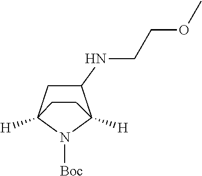 [SMILES: COCCNC1C[C@H]2CC[C@@H]1N2C(=O)OC(C)(C)C]

To a solution of tert-butyl (1S,4R)-2-oxo-7-azabicyclo [2.2.1] heptane-7-carboxylate (422 mg, 2 mmol) in MeOH (30 mL) was added 2-methoxyethan-1-amine (195 mg, 2.6 mmol) at room temperature, and the mixture was stirred at 55° C. for overnight. Upon completion, the mixture was cooled to room temperature and NaBH₄ (304 mg, 8 mmol) was added. The resulting mixture was stirred at room temperature for 1 h. Solvents were concentrated, and the residue was diluted with water (20 mL). Aqueous layer was extracted with DCM (30 mL *3), and the combined organic layer was concentrated to give a residue as the title compound (520 mg). MS (ESI, m/e) [M+1]⁺271.2.

### Step 2: (1S,4R)—N-(2-methoxyethyl)-7-azabicyclo [2.2.1] heptan-2-amine

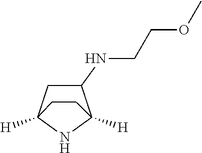

To a solution of tert-butyl (1S,4R)-2-((2-methoxyethyl) amino)-7-azabicyclo [2.2.1]heptane-7-carboxylate (520 mg, 1.92 mmol) in DCM (25 mL) was added HCl/dioxane (20 mL) at room temperature, and the mixture was stirred at room temperature for 1 h. The resulting mixture was concentrated to give a residue as the title compound (330 mg). MS (ESI, m/e) [M+1]⁺171.1.

### Step 3: (1S,4R)-7-(7-bromo-2-chloro-8-fluoro-6-(trifluoromethyl) quinazolin-4-yl)-N-(2-methoxyethyl)-7-azabicyclo [2.2.1] heptan-2-amine

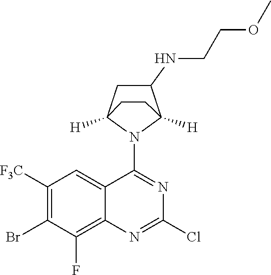 [SMILES: COCCNC1C[C@H]2CC[C@@H]1N2c1nc(Cl)nc2c(F)c(Br)c(C(F)(F)F)cc12]

To a solution of 7-bromo-2,4-dichloro-8-fluoro-6-(trifluoromethyl)quinazoline (470 mg, 1.3 mmol) in DCM (30 mL) was added (1S,4R)—N-(2-methoxyethyl)-7-azabicyclo [2.2.1] heptan-2-amine (278 mg, 1.625 mmol) and DIPEA (840 mg, 6.5 mmol) at room temperature, and the mixture was stirred at room temperature for 1 h. Solvent was concentrated and the residue was purified by chromatography column on silica (eluting with PE/EA=5/1) to give the title product (366 mg). MS (ESI, m/e) [M+1]⁺497.1.

### Step 4: tert-butyl ((1S,4R)-7-(7-bromo-2-chloro-8-fluoro-6-(trifluoromethyl) quinazolin-4-yl)-7-azabicyclo [2.2.1] heptan-2-yl) (2-methoxyethyl) carbamate

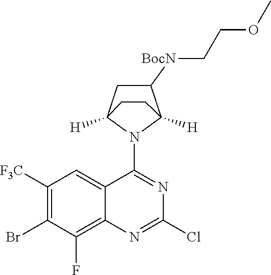 [SMILES: COCCN(C(=O)OC(C)(C)C)C1C[C@H]2CC[C@@H]1N2c1nc(Cl)nc2c(F)c(Br)c(C(F)(F)F)cc12]

To a solution of (1S,4R)-7-(7-bromo-2-chloro-8-fluoro-6-(trifluoromethyl) quinazolin-4-yl)-N-(2-methoxyethyl)-7-azabicyclo [2.2.1] heptan-2-amine (366 mg, 0.738 mmol) in DCM (20 mL) was added DIPEA (476 mg, 3.69 mmol) and (Boc)₂O (217 mg, 0.996 mmol) at room temperature, and the mixture was stirred at room temperature for 2 hrs. Solvent was concentrated and the residue was purified by chromatography column on silica (eluting with PE/EA=10/1) to give the title product (393 mg). MS (ESI, m/e) [M+1]⁺597.1.

### Step 5: tert-butyl ((1S,4R)-7-(7-bromo-2,8-difluoro-6-(trifluoromethyl) quinazolin-4-yl)-7-azabicyclo [2.2.1] heptan-2-yl) (2-methoxyethyl) carbamate

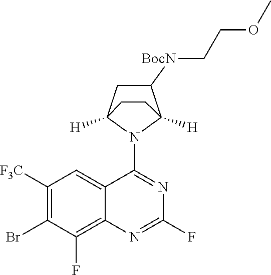 [SMILES: COCCN(C(=O)OC(C)(C)C)C1C[C@H]2CC[C@@H]1N2c1nc(F)nc2c(F)c(Br)c(C(F)(F)F)cc12]

To a solution of tert-butyl ((1S,4R)-7-(7-bromo-2-chloro-8-fluoro-6-(trifluoromethyl) quinazolin-4-yl)-7-azabicyclo [2.2.1] heptan-2-yl) (2-methoxyethyl) carbamate (393 mg, 0.66 mmol) in DMSO (15 mL) was added FK (573 mg, 9.89 mmol) at room temperature, and the mixture was stirred at 100° C. for overnight. The resulting mixture was diluted with water (20 mL) and extracted with DCM (30 mL *3), and the combined organic layer was concentrated to give a residue which was purified by chromatography column on silica (eluting with PE/EA=10/1) to give the title product (379 mg). MS (ESI, m/e) [M+1]⁺581.1.

### Step 6: tert-butyl ((1S,4R)-7-(7-bromo-8-fluoro-2-(((2R,7aS)-2-fluorotetrahydro-1H-pyrrolizin-7a(5H)-yl)methoxy)-6-(trifluoromethyl)quinazolin-4-yl)-7-azabicyclo[2.2.1]heptan-2-yl) (2-methoxyethyl) carbamate

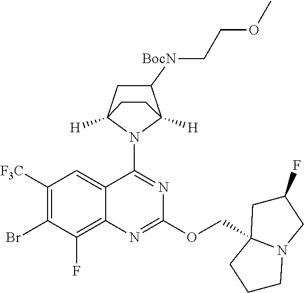 [SMILES: COCCN(C(=O)OC(C)(C)C)C1C[C@H]2CC[C@@H]1N2c1nc(OC[C@@]23CCCN2C[C@H](F)C3)nc2c(F)c(Br)c(C(F)(F)F)cc12]

NaH (30 mg, 0.75 mmol) was added to a solution of ((2R,7aS)-2-fluorotetrahydro-1H-pyrrolizin-7a(5H)-yl)methanol (71.55 mg, 0.45 mmol) in THF (10 mL), and the mixture was stirred at 0° C. for 0.5 h. Then, a solution of tert-butyl ((1S,4R)-7-(7-bromo-2,8-difluoro-6-(trifluoromethyl) quinazolin-4-yl)-7-azabicyclo [2.2.1] heptan-2-yl) (2-methoxyethyl) carbamate (174 mg, 0.3 mmol) in THF (10 mL) was added in and the mixture was stirred at room temperature for 1 h. The resulting mixture was quenched with ice water (5 mL) and extracted with EtOAc (10 mL*3), and the combined organic layer was concentrated to give a residue which was purified by chromatography column on silica (eluting with DCM/MeOH=10/1) to give the title product (143 mg). MS (ESI, m/e) [M+1]⁺720.2.

### Step 7: tert-butyl ((1S,4R)-7-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-7-azabicyclo [2.2.1]heptan-2-yl) (2-methoxyethyl)carbamate

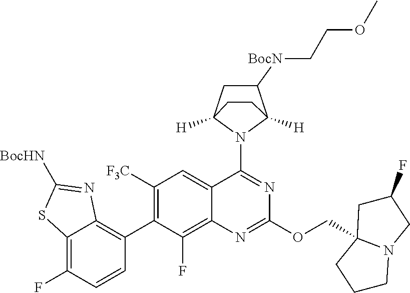 [SMILES: COCCN(C(=O)OC(C)(C)C)C1C[C@H]2CC[C@@H]1N2c1nc(OC[C@@]23CCCN2C[C@H](F)C3)nc2c(F)c(-c3ccc(F)c4sc(NC(=O)OC(C)(C)C)nc34)c(C(F)(F)F)cc12]

To a solution of tert-butyl ((1S,4R)-7-(7-bromo-8-fluoro-2-(((2R,7aS)-2-fluorotetrahydro-1H-pyrrolizin-7a(5H)-yl)methoxy)-6-(trifluoromethyl)quinazolin-4-yl)-7-azabicyclo[2.2.1]heptan-2-yl) (2-methoxyethyl) carbamate (143 mg, 0.199 mmol) in dioxane (15 mL) was added tert-butyl (7-fluoro-4-(4,4,5,5-tetramethyl-1,3,2-dioxaborolan-2-yl)benzo[d]thiazol-2-yl) carbamate (93 mg, 0.298 mmol), Pd(dppf)Cl₂ (16 mg, 0.02 mmol), NaHCO₃ (50 mg, 0.597 mmol) and water (3 mL), and the mixture was stirred at 100° C. for 2 hrs. The resulting mixture was concentrated and the crude product was purified by chromatography column on silica (eluting with DCM/MeOH=20/1) to give the crude product (85 mg). MS (ESI, m/e) [M+1]⁺808.3.

### Step 8: 7-fluoro-4-(8-fluoro-2-(((2R,7aS)-2-fluorotetrahydro-1H-pyrrolizin-7a(5H)-yl)methoxy)-4-((1S,4R)-2-((2-methoxyethyl)amino)-7-azabicyclo[2.2.1]heptan-7-yl)-6-(trifluoromethyl)quinazolin-7-yl) benzo[d]thiazol-2-amine

To a solution of tert-butyl ((1S,4R)-7-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl) quinazolin-4-yl)-7-azabicyclo [2.2.1]heptan-2-yl) (2-methoxyethyl)carbamate (85 mg, 0.094 mmol) in DCM (8 mL) was added TFA (4 mL) at room temperature, and the mixture was stirred at room temperature for 1 h. The resulting mixture was concentrated and pH was adjusted to 7 with Na₂CO₃. The organic layer was separated and solvents were removed to give a residue which was further purified by Prep-HPLC to give title product (28.27 mg, FA salt). ¹H NMR (500 MHz, MeOD) δ 8.22 (s, 1H), 7.21-7.18 (m, 1H), 6.99-6.96 (m, 1H), 5.55-5.44 (m, 1H), 5.08-5.07 (m, 1H), 4.96-4.93 (m, 1H), 4.62-4.55 (m, 2H), 3.87-3.64 (m, 4H), 3.61-3.59 (m, 2H), 3.42-3.33 (m, 4H), 3.08-2.98 (m, 2H), 2.66-2.45 (m, 3H), 2.40-2.20 (m, 3H), 2.16-1.96 (m, 4H), 1.81-1.78 (m, 1H), 1.43-1.40 (m, 1H). MS (ESI, m/e) [M+1]⁺708.5.

### Example 59: 4-(6-chloro-8-fluoro-2-(((2S,4R)-4-fluoro-1-methylpyrrolidin-2-yl)methoxy)-4-((1S,4R)-2-(methylamino)-7-azabicyclo[2.2.1]heptan-7-yl)quinazolin-7-yl)-7-fluorobenzo[d]thiazol-2-amine

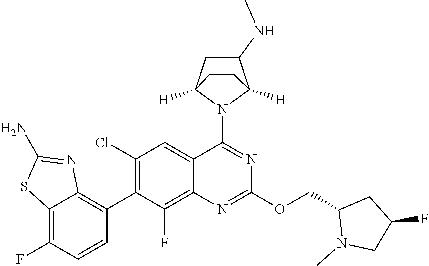 [SMILES: CNC1C[C@H]2CC[C@@H]1N2c1nc(OC[C@@H]2C[C@@H](F)CN2C)nc2c(F)c(-c3ccc(F)c4sc(N)nc34)c(Cl)cc12]

Synthetic Route

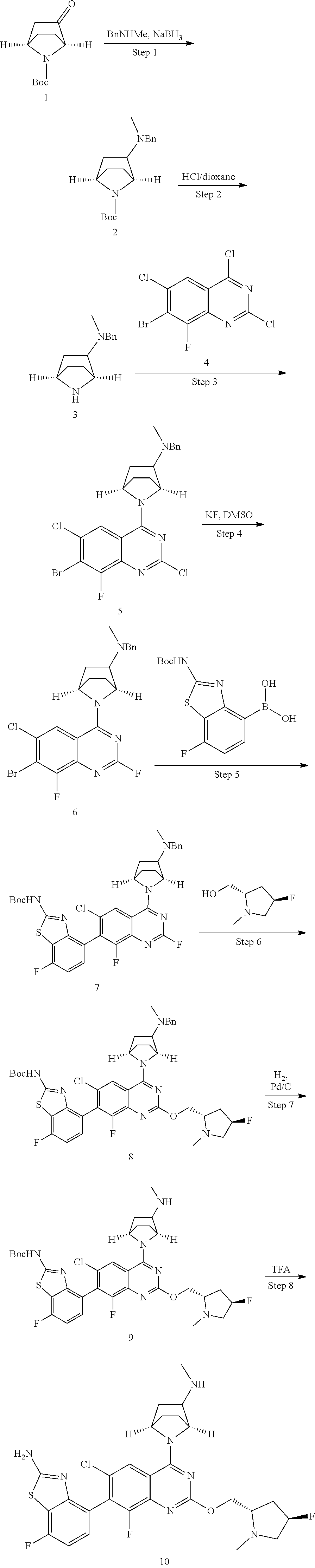

### Step 1: tert-butyl (1S,4R)-2-(benzyl(methyl)amino)-7-azabicyclo [2.2.1] heptane-7-carboxylate

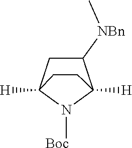 [SMILES: CN(Cc1ccccc1)C1C[C@H]2CC[C@@H]1N2C(=O)OC(C)(C)C]

To a solution of tert-butyl (1S,4R)-2-oxo-7-azabicyclo[2.2.1] heptane-7-carboxylate (600 mg, 2.84 mmol) in MeOH (30 mL) was N-methyl-1-phenylmethanamine (860 mg, 7.1 mmol) at room temperature, and the mixture was stirred at room temperature for 5 hrs. Then, NaBH₄ (304 mg, 8 mmol) was added in the resulting mixture and was stirred at room temperature for 1 h. The resulting mixture was concentrated, diluted with water (20 mL) and extracted with DCM (30 mL *3), and the combined organic layer was concentrated to give a residue (580 mg). MS (ESI, m/e) [M+1]⁺317.2.

### Step 2: (1S,4R)—N-benzyl-N-methyl-7-azabicyclo [2.2.1] heptan-2-amine

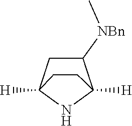 [SMILES: CN(Cc1ccccc1)C1C[C@H]2CC[C@@H]1N2]

To a solution of tert-butyl (1S,4R)-2-(benzyl(methyl)amino)-7-azabicyclo [2.2.1]heptane-7-carboxylate (580 mg, 1.83 mmol) in DCM (18 mL) was added TFA (6 mL) at room temperature, and the mixture was stirred at room temperature for 3 hrs. The resulting mixture was concentrated to give a residue (1300 mg). MS (ESI, m/e) [M+1]⁺217.1.

### Step 3: (1S,4R)—N-benzyl-7-(7-bromo-2,6-dichloro-8-fluoroquinazolin-4-yl)-N-methyl-7-azabicyclo [2.2.1] heptan-2-amine

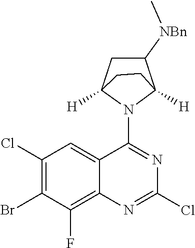 [SMILES: CN(Cc1ccccc1)C1C[C@H]2CC[C@@H]1N2c1nc(Cl)nc2c(F)c(Br)c(Cl)cc12]

To a solution of 7-bromo-2,4,6-trichloro-8-fluoroquinazoline (372 mg, 1.13 mmol) in DCM (35 mL) was added (1S,4R)—N-benzyl-N-methyl-7-azabicyclo [2.2.1] heptan-2-amine (1170 mg, 1.8 mmol) and DIPEA (1450 mg, 11.3 mmol) at room temperature, and the mixture was stirred at room temperature for 1 h. The resulting mixture was concentrated and the residue was purified by chromatography column on silica (eluting with PE/EA=5/1) to give the title product (148 mg). MS (ESI, m/e) [M+1]⁺509.0.

### Step 4: (1S,4R)—N-benzyl-7-(7-bromo-6-chloro-2,8-difluoroquinazolin-4-yl)-N-methyl-7-azabicyclo [2.2.1] heptan-2-amine

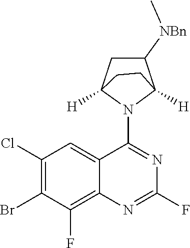 [SMILES: CN(Cc1ccccc1)C1C[C@H]2CC[C@@H]1N2c1nc(F)nc2c(F)c(Br)c(Cl)cc12]

To a solution of (1S,4R)—N-benzyl-7-(7-bromo-2,6-dichloro-8-fluoroquinazolin-4-yl)-N-methyl-7-azabicyclo [2.2.1] heptan-2-amine (148 mg, 0.29 mmol) in DMSO (15 mL) was added KF (253 mg, 4.37 mmol) at room temperature, and the mixture was stirred at 100° C. for overnight. The resulting mixture was diluted with water (20 mL) and extracted with DCM (30 mL *3), and the combined organic layer was concentrated to give a residue which was purified by chromatography column on silica (eluting with PE/EA=10/1) to give the title product (136 mg). MS (ESI, m/e) [M+1]⁺493.0.

### Step 5: tert-butyl (4-(4-((1S,4R)-2-(benzyl(methyl)amino)-7-azabicyclo[2.2.1]heptan-7-yl)-6-chloro-2,8-difluoroquinazolin-7-yl)-7-fluorobenzo[d]thiazol-2-yl) carbamate

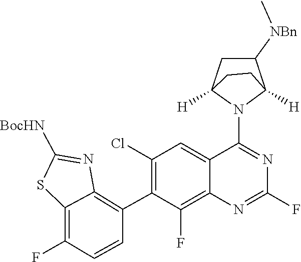 [SMILES: CN(Cc1ccccc1)C1C[C@H]2CC[C@@H]1N2c1nc(F)nc2c(F)c(-c3ccc(F)c4sc(NC(=O)OC(C)(C)C)nc34)c(Cl)cc12]

To a solution of (1S,4R)—N-benzyl-7-(7-bromo-6-chloro-2,8-difluoroquinazolin-4-yl)-N-methyl-7-azabicyclo [2.2.1] heptan-2-amine (126 mg, 0.256 mmol) in dioxane (8 mL) was added tert-butyl (7-fluoro-4-(4,4,5,5-tetramethyl-1,3,2-dioxaborolan-2-yl)benzo[d]thiazol-2-yl) carbamate (120 mg, 0.384 mmol), Pd(dppf)Cl₂ (21 mg, 0.0256 mmol), NaHCO₃ (64.5 mg, 0.768 mmol) and water (2 mL) at room temperature, and the mixture was stirred at 100° C. for 2 hrs. The resulting mixture was concentrated and the crude product was purified by chromatography column on silica (eluting with DCM/MeOH=20/1) to give the crude (94 mg). MS (ESI, m/e) [M+1]⁺681.2

### Step 6: tert-butyl (4-(4-((1S,4R)-2-(benzyl(methyl)amino)-7-azabicyclo[2.2.1]heptan-7-yl)-6-chloro-8-fluoro-2-(((2S,4R)-4-fluoro-1-methylpyrrolidin-2-yl)methoxy)quinazolin-7-yl)-7-fluorobenzo[d]thiazol-2-yl)carbamate

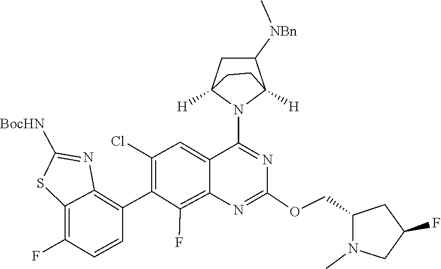 [SMILES: CN(Cc1ccccc1)C1C[C@H]2CC[C@@H]1N2c1nc(OC[C@@H]2C[C@@H](F)CN2C)nc2c(F)c(-c3ccc(F)c4sc(NC(=O)OC(C)(C)C)nc34)c(Cl)cc12]

NaH (8.3 mg, 0.207 mmol) was added to a solution of ((2S,4R)-4-fluoro-1-methylpyrrolidin-2-yl)methanol (13.8 mg, 0.104 mmol) in THF (8 mL), and the mixture was stirred at 0° C. for 0.5 h. Then, a solution of tert-butyl (4-(4-((1S,4R)-2-(benzyl(methyl)amino)-7-azabicyclo[2.2.1]heptan-7-yl)-6-chloro-2,8-difluoroquinazolin-7-yl)-7-fluorobenzo[d]thiazol-2-yl) carbamate (47 mg, 0.069 mmol) in THF (7 mL) was added in and the mixture was stirred at room temperature for 1 h. The resulting mixture was quenched with ice water (5 mL) and extracted with EtOAc (10 mL*3), and the combined organic layer was concentrated to give a residue which was purified by chromatography column on silica (eluting with DCM/MeOH=10/1) to give the title product (32 mg). MS (ESI, m/e) [M+1]⁺794.3.

### Step 7: tert-butyl (4-(6-chloro-8-fluoro-2-(((2S,4R)-4-fluoro-1-methylpyrrolidin-2-yl)methoxy)-4-((1S,4R)-2-(methylamino)-7-azabicyclo[2.2.1]heptan-7-yl)quinazolin-7-yl)-7-fluorobenzo[d]thiazol-2-yl)carbamate

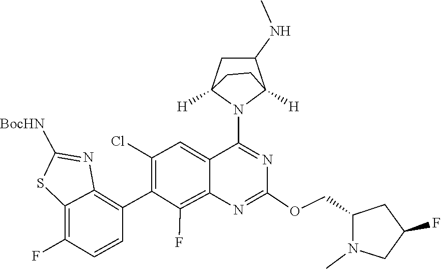 [SMILES: CNC1C[C@H]2CC[C@@H]1N2c1nc(OC[C@@H]2C[C@@H](F)CN2C)nc2c(F)c(-c3ccc(F)c4sc(NC(=O)OC(C)(C)C)nc34)c(Cl)cc12]

To a solution of tert-butyl (4-(4-((1S,4R)-2-(benzyl(methyl)amino)-7-azabicyclo[2.2.1]heptan-7-yl)-6-chloro-8-fluoro-2-(((2S,4R)-4-fluoro-1-methylpyrrolidin-2-yl)methoxy)quinazolin-7-yl)-7-fluorobenzo[d]thiazol-2-yl)carbamate (32 mg, 0.04 mmol) in MeOH (20 mL) was added Pd/C (16 mg) and 3 drops of HCl/dioxane at room temperature, and the mixture was stirred under hydrogen at room temperature for 3 h. The resulting mixture was concentrated to give a residue (35 mg). MS (ESI, m/e) [M+1]⁺704.2.

### Step 8: 4-(6-chloro-8-fluoro-2-(((2S,4R)-4-fluoro-1-methylpyrrolidin-2-yl)methoxy)-4-((1S,4R)-2-(methylamino)-7-azabicyclo[2.2.1]heptan-7-yl)quinazolin-7-yl)-7-fluorobenzo[d]thiazol-2-amine

To a solution of tert-butyl (4-(6-chloro-8-fluoro-2-(((2S,4R)-4-fluoro-1-methylpyrrolidin-2-yl)methoxy)-4-((1S,4R)-2-(methylamino)-7-azabicyclo[2.2.1]heptan-7-yl)quinazolin-7-yl)-7-fluorobenzo[d]thiazol-2-yl)carbamate (35 mg, 0.0498 mmol) in DCM (10 mL) was added TFA (2 mL) at room temperature, the mixture was stirred at room temperature for 1 h. The resulting mixture was concentrated at room temperature and pH was adjusted to 7 with Na₂CO₃, and the organic layer was concentrated to give a residue which was further purified by Prep-HPLC to give title product (10.88 mg, FA salt). ¹H NMR (500 MHz, MeOD) δ 8.02 (s, 1H), 7.23-7.20 (m, 1H), 7.02-6.98 (m, 1H), 5.35-5.24 (m, 1H), 5.11-5.10 (m, 1H), 4.96-4.95 (m, 1H), 4.71-4.68 (m, 1H), 4.63-4.57 (m, 1H), 3.87-3.82 (m, 1H), 3.75-3.69 (m, 1H), 3.62-3.54 (m, 1H), 3.09-3.01 (m, 1H), 2.80-2.78 (m, 6H), 2.60-2.55 (m, 1H), 2.50-2.39 (m, 1H), 2.22-2.08 (m, 3H), 1.97-1.93 (m, 1H), 1.81-1.78 (m, 1H), 1.59-1.50 (m, 1H). MS (ESI, m/e) [M+1]⁺604.3.

### Example 60: 4-(4-(3,8-diazabicyclo[3.2.1]octan-8-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

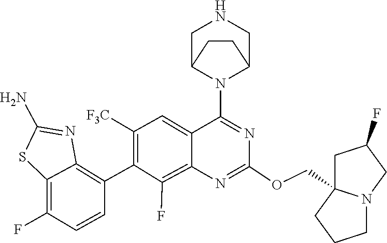 [SMILES: Nc1nc2c(-c3c(C(F)(F)F)cc4c(N5C6CCC5CNC6)nc(OC[C@@]56CCCN5C[C@H](F)C6)nc4c3F)ccc(F)c2s1]

Synthetic Route

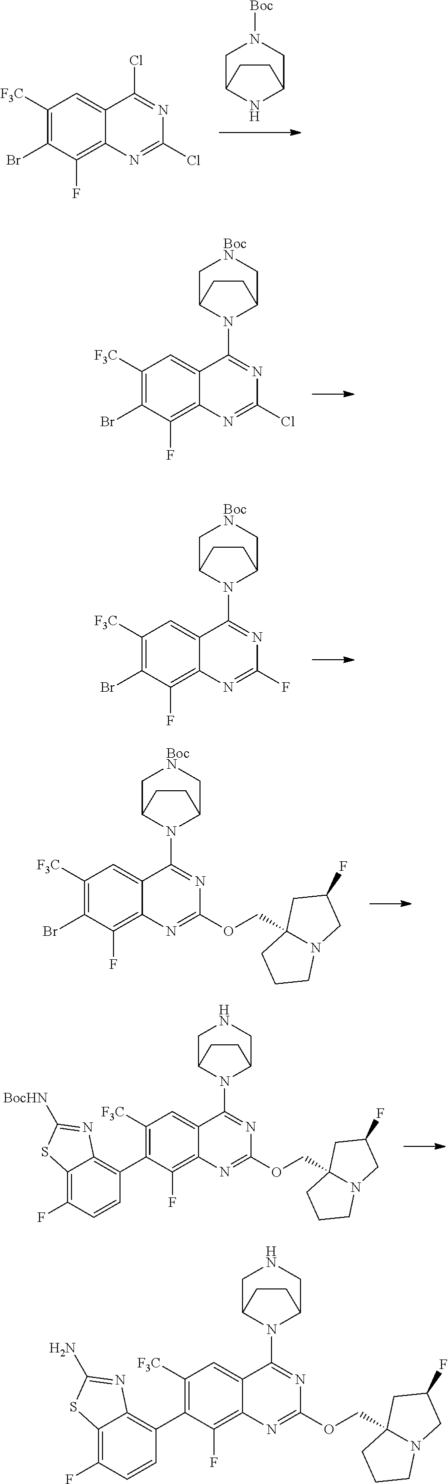

### Step 1: tert-butyl 8-(7-bromo-2-chloro-8-fluoro-6-(trifluoromethyl)quinazolin-4-yl)-3,8-diazabicyclo[3.2.1]octane-3-carboxylate

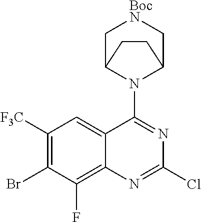 [SMILES: CC(C)(C)OC(=O)N1CC2CCC(C1)N2c1nc(Cl)nc2c(F)c(Br)c(C(F)(F)F)cc12]

To a solution of 7-bromo-2,4-dichloro-8-fluoro-6-(trifluoromethyl)quinazoline (400 mg, 1.1 mmol), tert-butyl 3,8-diazabicyclo[3.2.1]octane-3-carboxylate (280 mg, 1.3 mmol) in DCM (5 mL) was added TEA (222 mg, 2.2 mmol) at room temperature. The resulting mixture was stirred at room temperature for 16 hrs. After completion. The reaction mixture was purified by Prep-TLC (PE:EA=1:1) to give the title product (550 mg, crude). MS (ESI, m/e) [M+1]⁺539.1, 541.2.

### Step 2: tert-butyl 8-(7-bromo-2,8-difluoro-6-(trifluoromethyl)quinazolin-4-yl)-3,8-diazabicyclo[3.2.1]octane-3-carboxylate

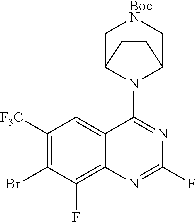 [SMILES: CC(C)(C)OC(=O)N1CC2CCC(C1)N2c1nc(F)nc2c(F)c(Br)c(C(F)(F)F)cc12]

To a solution of tert-butyl 8-(7-bromo-2-chloro-8-fluoro-6-(trifluoromethyl)quinazolin-4-yl)-3,8-diazabicyclo[3.2.1]octane-3-carboxylate (539 mg, 1.0 mmol) in DMSO (15 mL) was added KF (580 mg, 10 mmol) at room temperature. The resulting solution was stirred for 16 hrs at 110° C. After completion. The reaction mixture was diluted with EA (50 mL), washed with saturated NaCl (10 mL×3). The organic layer was dried over anhydrous Na₂SO₄, filtered and the filtrate was concentrated to give a residue. the residue was purified by silica gel column chromatography, eluted with EA to give the title product (200 mg). MS (ESI, m/e) [M+1]⁺523.2, 525.2.

### Step 3: tert-butyl 8-(7-bromo-8-fluoro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-3-carboxylate

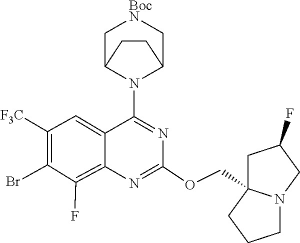 [SMILES: CC(C)(C)OC(=O)N1CC2CCC(C1)N2c1nc(OC[C@@]23CCCN2C[C@H](F)C3)nc2c(F)c(Br)c(C(F)(F)F)cc12]

To a solution of ((2R,7aS)-2-fluorotetrahydro-1H-pyrrolizin-7a(5H)-yl)methanol (121.1 mg, 0.76 mmol) in THF (5 mL) was added sodium hydride (43.8 mg, 1.14 mmol) at room temperature. The resulting mixture was stirred at room temperature for 1 hrs, Then tert-butyl 8-(7-bromo-2,8-difluoro-6-(trifluoromethyl)quinazolin-4-yl)-3,8-diazabicyclo[3.2.1]octane-3-carboxylate (200 mg, 0.38 mmol) was added to reaction mixture and stirred for 16 hrs at room temperature. After completion. The reaction mixture was purified by silica gel column chromatography, eluted with EtOAc to give the title product (70 mg). MS (ESI, m/e) [M+1]⁺662.3, 664.3.

### Step 4: tert-butyl (4-(4-(3,8-diazabicyclo[3.2.1]octan-8-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-yl)carbamate

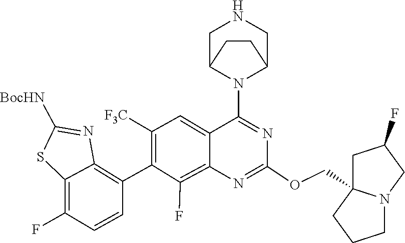 [SMILES: CC(C)(C)OC(=O)Nc1nc2c(-c3c(C(F)(F)F)cc4c(N5C6CCC5CNC6)nc(OC[C@@]56CCCN5C[C@H](F)C6)nc4c3F)ccc(F)c2s1]

To a solution of tert-butyl 8-(7-bromo-8-fluoro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-3-carboxylate (68.7 mg, 0.1 mmol), (2-amino-7-fluorobenzo[d]thiazol-4-yl)boronic acid (93.6 mg, 0.3 mmol) and Pd(dtbpf)Cl₂ (6.51 mg, 0.01 mmol) in dioxane (5 mL) and H₂O (1 mL) was added K₃PO₄ (63.6 mg, 0.3 mmol) at room temperature, The resulting mixture was stirred at 100° C. for 16 hrs under N₂ atmosphere. After completion. The reaction mixture was purified by Prep-TLC (DCM:MeOH=10:1) to give the title product (50 mg), MS (ESI, m/e) [M+1]⁺750.4.

### Step 5: 4-(4-(3,8-diazabicyclo[3.2.1]octan-8-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine, formic acid salt

To a solution of tert-butyl (4-(4-(3,8-diazabicyclo[3.2.1]octan-8-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-yl)carbamate (50 mg, 0.017 mmol) in DCM (5 mL) was added TFA (2 mL) at room temperature. The resulting mixture was stirred at room temperature for 6 hrs. After completion. The reaction mixture was concentrated to give the residue. The residue was purified by High-HPLC to give the title product (8.0 mg). ¹H NMR (500 MHz, CD₃OD) δ 8.14 (s, 1H), 8.09 (s, 1H), 7.89 (s, 2H), 7.20-7.17 (m, 1H), 7.06-7.03 (m, 1H), 5.36-5.25 (m, 1H), 4.94 (s, 2H), 4.18-4.09 (m, 2H), 3.32-3.29 (m, 2H), 3.16-3.11 (m, 5H), 2.88-2.87 (m, 1H), 2.16-2.07 (m, 2H), 2.02-2.00 (m, 5H), 1.88-1.81 (m, 3H). 6 MS (ESI, m/e) [M+1]⁺650.4.

### Example 61: 4-(4-(3,8-diazabicyclo [3.2.1] octan-3-yl)-6-chloro-2-((1,2-dimethylpyrrolidin-2-yl) methoxy)-8-fluoroquinazolin-7-yl)-7-fluorobenzo[d]thiazol-2-amine

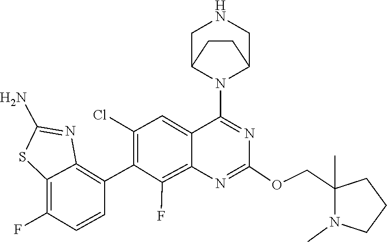 [SMILES: CN1CCCC1(C)COc1nc(N2C3CCC2CNC3)c2cc(Cl)c(-c3ccc(F)c4sc(N)nc34)c(F)c2n1]

Synthetic Route

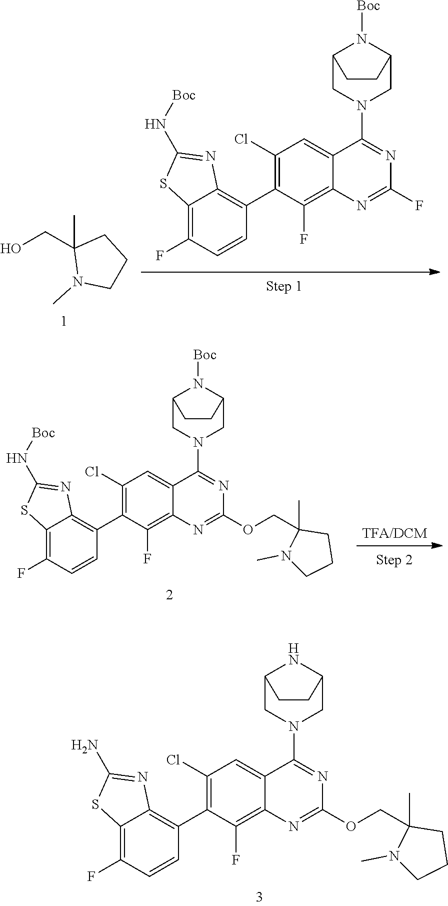

### Step 1: tert-butyl-3-(7-(2-((tert-butoxycarbonyl)amino)-7-fluorobenzo[d]thiazol-4-yl)-6-chloro-2-((1,2-dimethylpyrrolidin-2-yl)methoxy)-8-fluoroquinazolin-4-yl)-3,8-diazabicyclo[3.2.1]octane-8-carboxylate

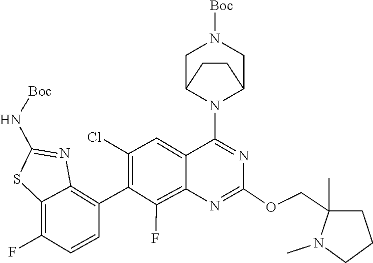 [SMILES: CN1CCCC1(C)COc1nc(N2C3CCC2CN(C(=O)OC(C)(C)C)C3)c2cc(Cl)c(-c3ccc(F)c4sc(NC(=O)OC(C)(C)C)nc34)c(F)c2n1]

NaH (6.7 mg, 0.168 mmol) was added to a solution of (1,2-dimethylpyrrolidin-2-yl) methanol (12.9 mg, 0.1 mmol) in THF (5 mL), and the mixture was stirred at 0° C. for 0.5 h. Then a solution of tert-butyl 3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (45 mg, 0.067 mmol) in THF (5 mL) was added in and the mixture was stirred at room temperature for 1 h. The resulting mixture was quenched with ice water (10 mL) and extracted with EtOAc (15 mL*3), and the combined organic layer was concentrated to give a residue which was purified by chromatography column on silica (eluting with DCM/MeOH=10/1) to give the title product (29 mg). MS (ESI, m/e) [M+1]⁺786.3.

### Step 2: 4-(4-(3,8-diazabicyclo [3.2.1] octan-3-yl)-6-chloro-2-((1,2-dimethylpyrrolidin-2-yl) methoxy)-8-fluoroquinazolin-7-yl)-7-fluorobenzo[d]thiazol-2-amine

To a solution of t tert-butyl-3-(7-(2-((tert-butoxycarbonyl)amino)-7-fluorobenzo[d]thiazol-4-yl)-6-chloro-2-((1,2-dimethylpyrrolidin-2-yl)methoxy)-8-fluoroquinazolin-4-yl)-3,8-diazabicyclo [3.2.1] octane-8-carboxylate (29 mg, 0.037 mmol) in DCM (10 mL) was added TFA (4 mL) at room temperature, and the mixture was stirred at room temperature for 1 h. The resulting mixture was concentrated at room temperature and pH was adjusted to 7 with Na₂CO₃, and the organic layer was concentrated to give a residue which was further purified by Prep-HPLC to give the title product (11.58 mg, FA salt). ¹H NMR (500 MHz, MeOD) δ 7.93 (s, 1H), 7.23-7.20 (m, 1H), 7.02-6.99 (m, 1H), 4.73-4.61 (m, 4H), 4.21 (s, 2H), 3.90-3.85 (m, 2H), 3.70-3.65 (m, 1H), 3.44-3.42 (m, 1H), 2.95 (s, 3H), 2.40-2.31 (m, 1H), 2.22-2.06 (m, 7H), 1.52 (s, 3H). MS (ESI, m/e) [M+1]⁺586.3.

### Example 62: 4-(4-(3,8-diazabicyclo[3.2.1]octan-3-yl)-6-chloro-8-fluoro-2-(3-methyl-3-(piperidin-1-yl)azetidin-1-yl)quinazolin-7-yl)-7-fluorobenzo[d]thiazol-2-amine

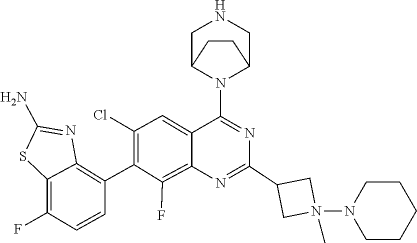

Synthetic Route

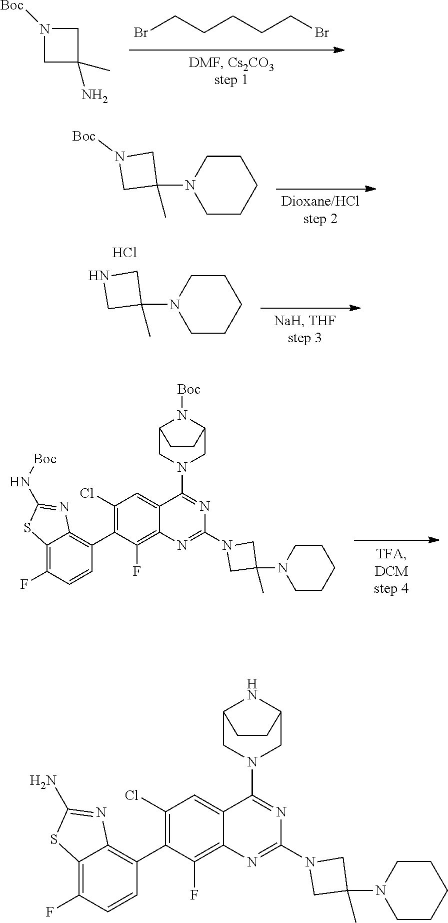

### Step 1: tert-butyl 3-methyl-3-(piperidin-1-yl)azetidine-1-carboxylate

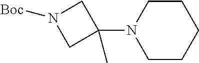 [SMILES: CC(C)(C)OC(=O)N1CC(C)(N2CCCCC2)C1]

A mixture of tert-butyl 3-amino-3-methylazetidine-1-carboxylate (930 mg, 5 mmol), 1,5-dibromopentane (1.15 g, 5 mmol) and Cs₂CO₃ (6.4 g, 19 mmol) in DMF (20 mL) was stirred at room temperature for overnight. After completion, the resulting mixture was poured into water and extracted with EtOAc. The organic layer was concentrated under vacuum and crude product (800 mg) was used directly in the next step. MS (ESI, m/e) [M+1]⁺255.4

### Step 2: 1-(3-methylazetidin-3-yl)piperidine hydrogen chloride

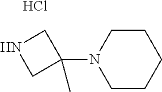

A solution of tert-butyl 3-methyl-3-(piperidin-1-yl)azetidine-1-carboxylate (800 mg) in dioxane/HCl (4M) (10 mL) was stirred at room temperature for 2 hours. After completion, the mixture was concentrated under vacuum and crude product (600 mg) was used directly in the next step. MS (ESI, m/e) [M+1]⁺155.3

### Step 3: tert-butyl-3-(7-(2-((tert-butoxycarbonyl)amino)-7-fluorobenzo[d]thiazol-4-yl)-6-chloro-8-fluoro-2-(3-methyl-3-(piperidin-1-yl)azetidin-1-yl)quinazolin-4-yl)-3,8-diazabicyclo[3.2.1]octane-8-carboxylate

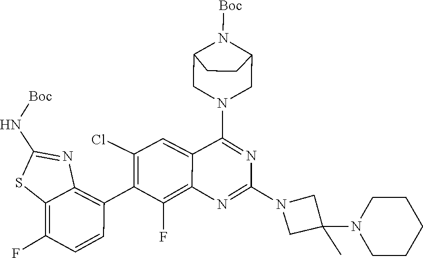 [SMILES: CC(C)(C)OC(=O)Nc1nc2c(-c3c(Cl)cc4c(N5CC6CCC(C5)N6C(=O)OC(C)(C)C)nc(N5CC(C)(N6CCCCC6)C5)nc4c3F)ccc(F)c2s1]

To a solution of 1-(3-methylazetidin-3-yl)piperidine hydrogen chloride (600 mg) in THF (10 mL) was added NaH (50 mg). The mixture was stirred at room temperature for 30 mins and then tert-butyl-3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (70 mg, 0.1 mmol) was added. The reaction mixture was stirred for 2 hours at room temperature. After completion, the reaction was quenched by water. The resulting mixture was evaporated and the crude product was purified by silica column (eluting with DCM/MeOH=20/1) to give the title product (30 mg). MS (ESI, m/e) MS (ESI, m/e) 811.4

### Step 4: 4-(4-(3,8-diazabicyclo[3.2.1]octan-3-yl)-6-chloro-8-fluoro-2-(3-methyl-3-(piperidin-1-yl)azetidin-1-yl)quinazolin-7-yl)-7-fluorobenzo[d]thiazol-2-amine

A mixture of tert-butyl-3-(7-(2-((tert-butoxycarbonyl)amino)-7-fluorobenzo[d]thiazol-4-yl)-6-chloro-8-fluoro-2-(3-methyl-3-(piperidin-1-yl)azetidin-1-yl)quinazolin-4-yl)-3,8-diazabicyclo[3.2.1]octane-8-carboxylate (30 mg, 0.037 mmol) in DCM (2 mL) and TFA (1 mL) was stirred at room temperature for 2 hours. After completion, the resulting mixture was directly concentrated and the residues adjusted to pH=9 with DIPEA. Then the residues were purified by C18 column (eluting with H₂O/CH₃CN=3/1) to afford the title product (21.7 mg, FA salt). ¹H NMR (500 MHz, CD₃OD) δ 8.49 (s, 1H), 7.71 (s, 1H), 7.20-7.16 (m, 1H), 7.02-6.96 (m, 1H), 4.51-4.35 (m, 2H), 4.16-3.99 (m, 4H), 3.86-3.92 (m, 2H), 3.41-3.72 (m, 2H), 2.62 (s, 4H), 2.07 (s, 4H), 1.81-1.67 (m, 4H), 1.54 (s, 2H), 1.46 (s, 3H). MS (ESI, m/e) [M+H]⁺ 611.40.

### Example 63: 4-(4-(3,8-diazabicyclo[3.2.1]octan-3-yl)-6,8-dichloro-2-(((2R,7aS)-2-fluorotetrahydro-1H-pyrrolizin-7a(5H)-yl)methoxy)quinazolin-7-yl)-7-fluorobenzo[d]thiazol-2-amine

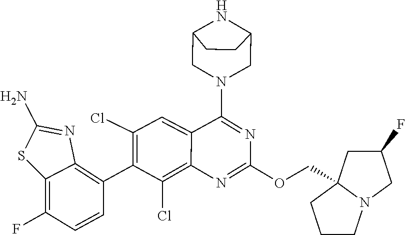 [SMILES: Nc1nc2c(-c3c(Cl)cc4c(N5CC6CCC(C5)N6)nc(OC[C@@]56CCCN5C[C@H](F)C6)nc4c3Cl)ccc(F)c2s1]

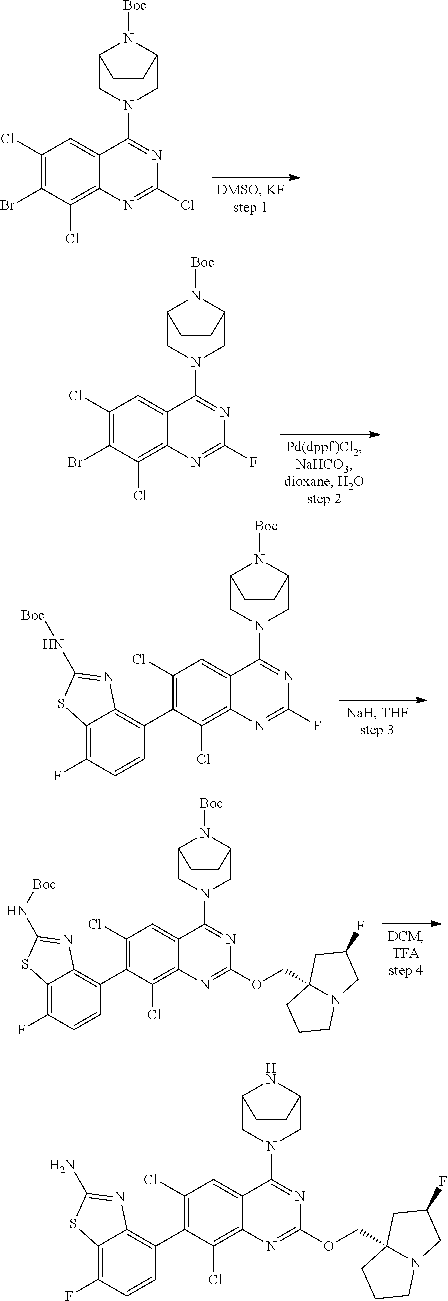

### Step 1: tert-butyl-3-(7-bromo-6,8-dichloro-2-fluoroquinazolin-4-yl)-3,8-diazabicyclo[3.2.1]octane-8-carboxylate

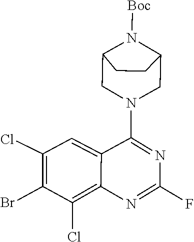 [SMILES: CC(C)(C)OC(=O)N1C2CCC1CN(c1nc(F)nc3c(Cl)c(Br)c(Cl)cc13)C2]

To a mixture of tert-butyl-3-(7-bromo-2,6,8-trichloroquinazolin-4-yl)-3,8-diazabicyclo[3.2.1]octane-8-carboxylate (1 g, 2 mmol) in DMSO (10 mL) was added KF (1 g) and the mixture was stirred at 100° C. for 8 hours. After completion, the resulting mixture was poured into water and extracted with EtOAc. The organic layer was combined and concentrated. Then the crude product was further purified by silica column (eluting with PE/EA=2/1) to give the title product (800 mg). MS (ESI, m/e) [M+1]⁺505.3

### Step 2: tert-butyl-3-(7-(2-((tert-butoxycarbonyl)amino)-7-fluorobenzo[d]thiazol-4-yl)-6,8-dichloro-2-fluoroquinazolin-4-yl)-3,8-diazabicyclo[32.1]octane-8-carboxylate

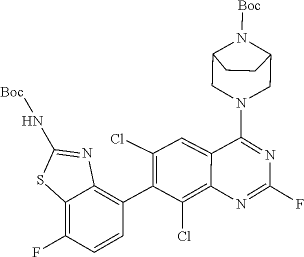 [SMILES: CC(C)(C)OC(=O)Nc1nc2c(-c3c(Cl)cc4c(N5CC6CCC(C5)N6C(=O)OC(C)(C)C)nc(F)nc4c3Cl)ccc(F)c2s1]

To a solution of tert-butyl-3-(7-bromo-6,8-dichloro-2-fluoroquinazolin-4-yl)-3,8-diazabicyclo[3.2.1]octane-8-carboxylate (600 mg, 1.2 mmol) in dioxane (50 mL) and H₂O (10 mL) was added Pd(dppf)Cl₂ (300 mg), NaHCO₃ (600 mg) and (2-((tert-butoxycarbonyl)amino)-7-fluorobenzo[d]thiazol-4-yl)boronic acid (400 mg, 1.2 mmol) stepwise. The reaction mixture was stirred at 100° C. for 2 h. After completion, solvents were evaporated and the crude product was purified by silica column (eluting with PE/EtOAc=2/1) to give the title product (500 mg). MS (ESI, m/e) [M+1]⁺693.3

### Step 3: tert-butyl-3-(7-(2-((tert-butoxycarbonyl)amino)-7-fluorobenzo[d]thiazol-4-yl)-6,8-dichloro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

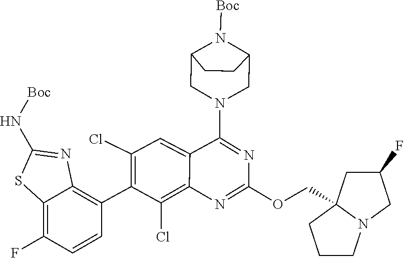 [SMILES: CC(C)(C)OC(=O)Nc1nc2c(-c3c(Cl)cc4c(N5CC6CCC(C5)N6C(=O)OC(C)(C)C)nc(OC[C@@]56CCCN5C[C@H](F)C6)nc4c3Cl)ccc(F)c2s1]

To a solution of ((2S,4R)-4-fluoro-1-methylpyrrolidin-2-yl)methanol (60 mg, 0.38 mmol) in THF (10 mL) was added NaH (60 mg). The mixture was stirred for 30 mins at room temperature and then tert-butyl-3-(7-(2-((tert-butoxycarbonyl)amino)-7-fluorobenzo[d]thiazol-4-yl)-6,8-dichloro-2-fluoroquinazolin-4-yl)-3,8-diazabicyclo[3.2.1]octane-8-carboxylate (200 mg, 0.29 mmol) was added. The reaction mixture was stirred at room temperature for 2 hours. After completion, the reaction was quenched by water. Solvents were evaporated and the crude product was purified by silica column (eluting with DCM/MeOH=9/1) to give the title product (120 mg). MS (ESI, m/e) [M+1]⁺832.4.

### Step 4: 4-(4-(3,8-diazabicyclo[3.2.1]octan-3-yl)-6,8-dichloro-2-(((2R,7aS)-2-fluorotetrahydro-1H-pyrrolizin-7a(5H)-yl)methoxy)quinazolin-7-yl)-7-fluorobenzo[d]thiazol-2-amine

A mixture of tert-butyl-3-(7-(2-((tert-butoxycarbonyl)amino)-7-fluorobenzo[d]thiazol-4-yl)-6,8-dichloro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (120 mg, 0.14 mmol) in DCM (4 mL) and TFA (2 mL) was stirred for 2 hours at room temperature. After completion, the resulting mixture was evaporated and the residue was adjusted to pH=9 with DIPEA. Then the residues were purified by C18 column (eluting with H₂O/CH₃CN=3/1) to afford the title product (52 mg, FA salt). ¹H NMR (400 MHz, CD₃OD) δ 8.46 (s, 1H), 8.04 (s, 1H), 7.16-7.10 (m, 1H), 7.04-6.98 (m, 1H), 5.30-5.49 (m, 1H), 4.66-4.49 (m, 4H), 4.03 (s, 2H), 3.86-3.62 (m, 5H), 2.63-1.88 (m, 11H). MS (ESI, m/e) [M+H]⁺ 632.3.

### Example 64: 4-(4-(3,8-diazabicyclo[3.2.1]octan-3-yl)-8-chloro-2-(((2R,7aS)-2-fluorotetrahydro-1H-pyrrolizin-7a(5H)-yl)methoxy)-6-(trifluoromethyl)quinazolin-7-yl)-7-fluorobenzoI[d]thiazol-2-amine

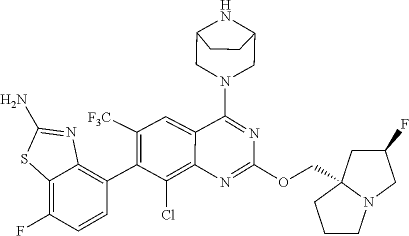 [SMILES: Nc1nc2c(-c3c(C(F)(F)F)cc4c(N5CC6CCC(C5)N6)nc(OC[C@@]56CCCN5C[C@H](F)C6)nc4c3Cl)ccc(F)c2s1]

Synthetic Route

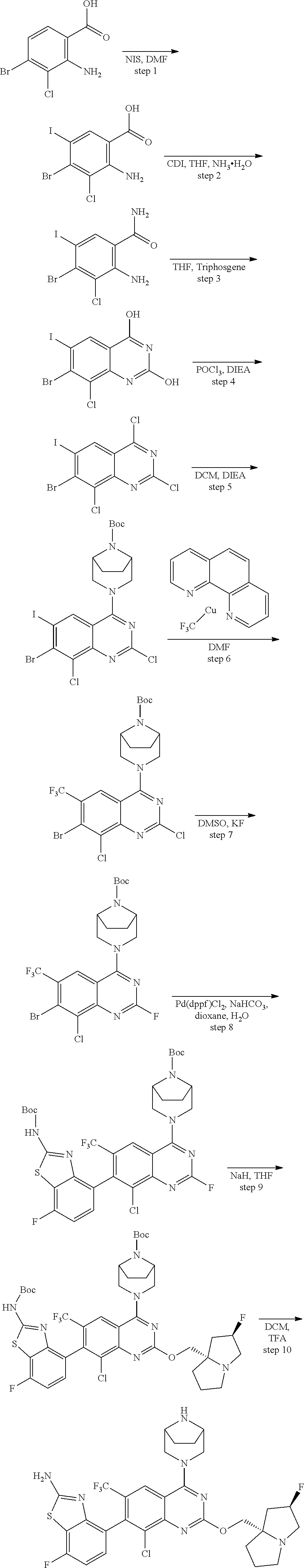

### Step 1: 2-amino-4-bromo-3-chloro-5-iodobenzoic acid

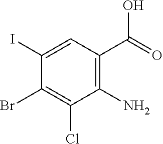 [SMILES: Nc1c(C(=O)O)cc(I)c(Br)c1Cl]

To a mixture of 2-amino-4-bromo-3-chlorobenzoic acid (13 g, 52 mmol) in DMF (100 mL) was added NIS (20 g, 89 mmol) and the mixture was stirred for 2 hours at 70° C. After completion, the resulting mixture was poured into water and filtered. The solid was washed with PE/EtOAc=1/1 to afford the title product (18 g). MS (ESI, m/e) [M+1]⁺375.9.

### Step 2: 2-amino-4-bromo-3-chloro-5-iodobenzamide

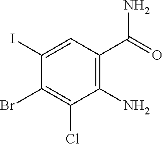 [SMILES: NC(=O)c1cc(I)c(Br)c(Cl)c1N]

To a solution of 2-amino-4-bromo-3-chloro-5-iodobenzoic acid (6 g, 16 mmol) in THF (100 mL) was added CDI (6 g). The mixture was stirred for 3 hours at room temperature and then NH₃·H₂O (20 mL) was added. The reaction mixture was stirred for 1 hour at room temperature. After completion, solvents were concentrated and the crude product was purified by silica column (eluting with EtOAc) to give the title product (5 g). MS (ESI, m/e) [M+1]⁺374.9.

### Step 3: 7-bromo-8-chloro-6-iodoquinazoline-2,4-diol

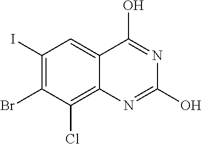 [SMILES: Oc1nc(O)c2cc(I)c(Br)c(Cl)c2n1]

To a mixture of 2-amino-4-bromo-3-chloro-5-iodobenzamide (5 g, 13 mmol) in THF (100 mL) was added triphosgene (2 g). The reaction mixture was stirred for 2 hours at room temperature. After completion, the reaction was quenched by water and filtered. The solid was evaporated under vacuum to give the title product (5.2 g). MS (ESI, m/e) [M+H]⁺ 401.1.

### Step 4: 7-bromo-2,4,8-trichloro-6-iodoquinazoline

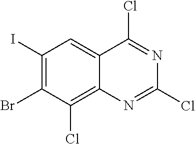 [SMILES: Clc1nc(Cl)c2cc(I)c(Br)c(Cl)c2n1]

To a mixture of 7-bromo-6,8-dichloroquinazoline-2,4-diol (5.2 g, 13 mmol) in POCl₃ (50 mL) was added DIPEA (10 mL) and the mixture was stirred for 2 hours at 120° C. After completion, the resulting mixture was poured into ice-water and extracted with DCM. The organic layer was concentrated, and the crude product was further purified by silica column (eluting with PE/EtOAc=5/1) to afford the title product (5 g). MS (ESI, m/e) [M+1]⁺346.8.

### Step 5: tert-butyl-3-(7-bromo-2,8-dichloro-6-iodoquinazolin-4-yl)-3,8-diazabicyclo[3.2.1]octane-8-carboxylate

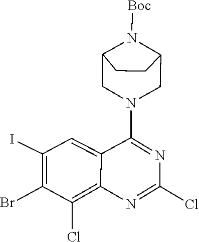 [SMILES: CC(C)(C)OC(=O)N1C2CCC1CN(c1nc(Cl)nc3c(Cl)c(Br)c(I)cc13)C2]

A mixture of 7-bromo-2,4,8-trichloro-6-iodoquinazoline (3 g, 8.7 mmol), tert-butyl-3,8-diazabicyclo[3.2.1]octane-8-carboxylate (1.5 g, 8.7 mmoL) in DCM (100 mL) and DIPEA (6 mL) was stirred for 2 hours at room temperature. After completion, solvents were evaporated and the crude product was purified by silica column (eluting with PE/EtOAc=3/1) to afford the title product (3 g). MS (ESI, m/e) [M+1]⁺613.2.

### Step 6: tert-butyl-3-(7-bromo-2,8-dichloro-6-(trifluoromethyl)quinazolin-4-yl)-3,8-diazabicyclo[3.2.1]octane-8-carboxylate

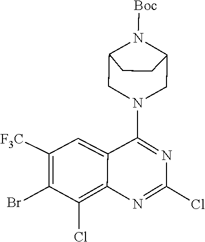 [SMILES: CC(C)(C)OC(=O)N1C2CCC1CN(c1nc(Cl)nc3c(Cl)c(Br)c(C(F)(F)F)cc13)C2]

A mixture of tert-butyl-3-(7-bromo-2,8-dichloro-6-iodoquinazolin-4-yl)-3,8-diazabicyclo[3.2.1]octane-8-carboxylate (3 g, 4.9 mmol), 1,10-phenanthroline (trifluoromethyl)copper (4.5 g, 14.4 mmol) in DMF (50 mL) was stirred for 30 mins at 80° C. After completion, the resulting mixture was poured into water and extracted with EtOAc. The organic layer was concentrated, and the residue was further purified by silica column (eluting with PE/EtOAc=3/1) to afford the title product (1.5 g). MS (ESI, m/e) [M+1]⁺555.3

### Step 7: tert-butyl-3-(7-bromo-8-chloro-2-fluoro-6-(trifluoromethyl)quinazolin-4-yl)-3,8-diazabicyclo[3.2.1]octane-8-carboxylate

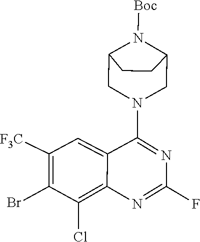 [SMILES: CC(C)(C)OC(=O)N1C2CCC1CN(c1nc(F)nc3c(Cl)c(Br)c(C(F)(F)F)cc13)C2]

To a mixture of tert-butyl-3-(7-bromo-2,8-dichloro-6-(trifluoromethyl)quinazolin-4-yl)-3,8-diazabicyclo[3.2.1]octane-8-carboxylate (1.5 g, 2.7 mmol) in DMSO (20 mL) was added KF (3 g) and the mixture was stirred for 3 hours at 100° C. After completion, the resulting mixture was poured into water and extracted with EtOAc. The organic layer was combined and concentrated. Then the residue was further purified by silica column (eluting with PE/EA=4/1) to give the title product (1.2 g). MS (ESI, m/e) [M+1]⁺539.2

### Step 8: tert-butyl-3-(7-(2-((tert-butoxycarbonyl)amino)-7-fluorobenzo[d]thiazol-4-yl)-8-chloro-2-fluoro-6-(trifluoromethyl)quinazolin-4-yl)-3,8-diazabicyclo[3.2.1]octane-8-carboxylate

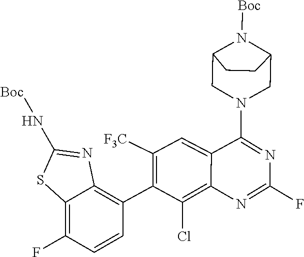 [SMILES: CC(C)(C)OC(=O)Nc1nc2c(-c3c(C(F)(F)F)cc4c(N5CC6CCC(C5)N6C(=O)OC(C)(C)C)nc(F)nc4c3Cl)ccc(F)c2s1]

To a solution of tert-butyl-3-(7-bromo-8-chloro-2-fluoro-6-(trifluoromethyl)quinazolin-4-yl)-3,8-diazabicyclo[3.2.1]octane-8-carboxylate (200 mg, 0.4 mmol) in dioxane (10 mL) and H₂O (2 mL) was added Pd(dppf)Cl₂ (100 mg), NaHCO₃ (300 mg) and (2-((tert-butoxycarbonyl)amino)-7-fluorobenzo[d]thiazol-4-yl)boronic acid (180 mg, 0.6 mmol) stepwise. The reaction mixture was stirred for 40 mins at 100° C. After completion, solvents were concentrated and the residues was purified by silica column (eluting with PE/EtOAc=3/1) to give the title product (100 mg). MS (ESI, m/e) [M+1]⁺727.3

### Step 9: tert-butyl-3-(7-(2-((tert-butoxycarbonyl)amino)-7-fluorobenzo[d]thiazol-4-yl)-8-chloro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate

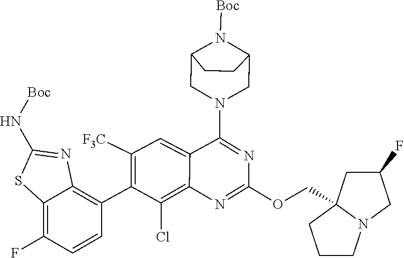 [SMILES: CC(C)(C)OC(=O)Nc1nc2c(-c3c(C(F)(F)F)cc4c(N5CC6CCC(C5)N6C(=O)OC(C)(C)C)nc(OC[C@@]56CCCN5C[C@H](F)C6)nc4c3Cl)ccc(F)c2s1]

To a solution of ((2S,4R)-4-fluoro-1-methylpyrrolidin-2-yl)methanol (20 mg, 0.13 mmol) in THF (10 mL) was added NaH (30 mg). The mixture was stirred for 30 mins at room temperature and then tert-butyl-3-(7-(2-((tert-butoxycarbonyl)amino)-7-fluorobenzo[d]thiazol-4-yl)-8-chloro-2-fluoro-6-(trifluoromethyl)quinazolin-4-yl)-3,8-diazabicyclo[3.2.1]octane-8-carboxylate (76 mg, 0.1 mmol) was added. The reaction mixture was stirred for 2 hours at room temperature. After completion, the reaction was quenched by H₂O. Solvents were evaporated and the crude product was purified by silica column (eluting with DCM/MeOH=20/1) to give the title product (60 mg). MS (ESI, m/e) [M+1]⁺866.3.

### Step 9: 4-(4-(3,8-diazabicyclo[3.2.1]octan-3-yl)-8-chloro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

A mixture of tert-butyl-3-(7-(2-((tert-butoxycarbonyl)amino)-7-fluorobenzo[d]thiazol-4-yl)-8-chloro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate (60 mg, 0.07 mmol) in DCM (4 mL) and TFA (2 mL) was stirred for 2 hours at room temperature. After completion, the resulting mixture was directly concentrated and the residue was adjusted to pH=9 with DIPEA. Then the residues were purified by C18 column (eluting with H₂O/CH₃CN=2/1) to afford the title product (35 mg, FA salt). ¹H NMR (500 MHz, CD₃OD) δ 8.52 (s, 1H), 8.02 (s, 1H), 7.13-7.08 (m, 1H), 7.03-6.97 (m, 1H), 5.38-5.50 (m, 1H), 4.59-4.39 (m, 4H), 3.88 (s, 2H), 3.82-3.62 (m, 2H), 3.36-3.32 (m, 1H), 2.52-2.24 (m, 3H), 2.22-2.05 (m, 2H), 1.96 (s, 5H). MS (ESI, m/e) [M+1]⁺666.4.

### Example 65: 6-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-4-chloro-5-(trifluoromethyl)pyridin-2-amine

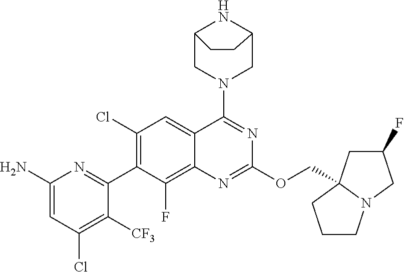 [SMILES: Nc1cc(Cl)c(C(F)(F)F)c(-c2c(Cl)cc3c(N4CC5CCC(C4)N5)nc(OC[C@@]45CCCN4C[C@H](F)C5)nc3c2F)n1]

Synthetic Route

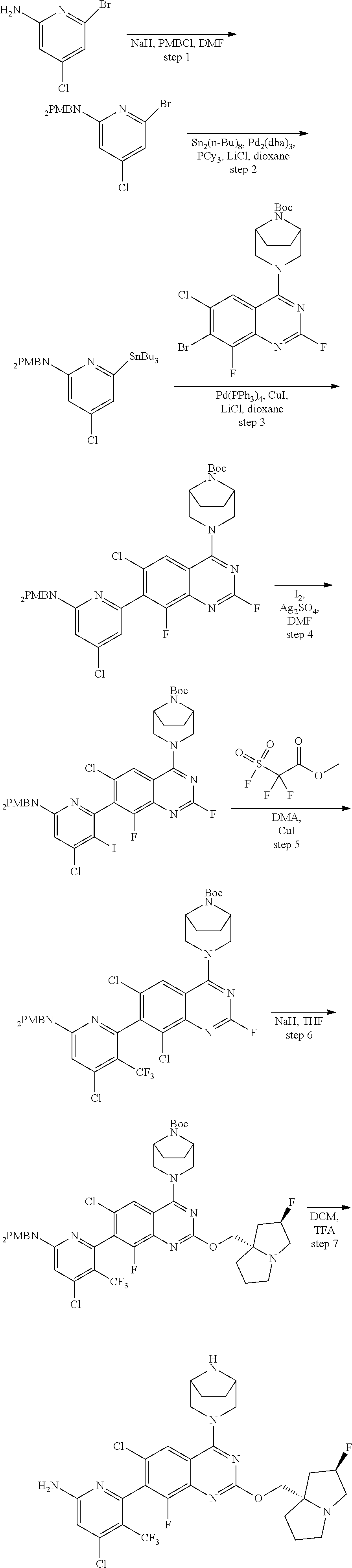

### Step 1: 6-bromo-4-chloro-N,N-bis(4-methoxybenzyl)pyridin-2-amine

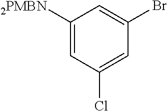

To a solution of 6-bromo-4-chloropyridin-2-amine (5 g, 24 mmol) in DMF (50 mL) was added NaH (3.7 g). The mixture was stirred for 1 h at room temperature and then PMBCl (8.4 g, 53 mmol) was added. The reaction mixture was stirred for 2 hours at room temperature. After completion, the reaction was quenched by H₂O. The mixture was poured into water and filtered To obtain solid as the title product (10 g). MS (ESI, m/e) [M+1]⁺447.2.

### Step 2: 4-chloro-N,N-bis(4-methoxybenzyl)-6-(tributylstannyl)pyridin-2-amine

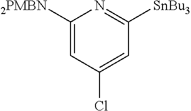

To a mixture of 6-bromo-4-chloro-N,N-bis(4-methoxybenzyl)pyridin-2-amine (4.47 g, 10 mmol), Sn₂(n-Bu)₆ (6.5 mL, 12 mmol), Pd₂(dba)₃ (915 mg, 1 mmol), LiCl (2.1 g) in dioxane (100 mL) was added PCy₃ (560 mg, 4 mmol) and the mixture was stirred for 16 hours at 110° C. After completion, the resulting mixture was directly concentrated. Then, the residue was further purified by silica column (eluting with PE/EtOAc=10/1) to give the title product (6 g). MS (ESI, m/e) [M+1]⁺659.2

### Step 3: tert-butyl-3-(7-(6-(bis(4-methoxybenzyl)amino)-4-chloropyridin-2-yl)-6-chloro-2,8-difluoroquinazolin-4-yl)-3,8-diazabicyclo[3.2.1]octane-8-carboxylate

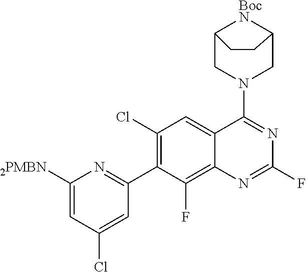

A mixture of 4-chloro-N,N-bis(4-methoxybenzyl)-6-(tributylstannyl)pyridin-2-amine (6 g, 9 mmol), tert-butyl-3-(7-bromo-6-chloro-2,8-difluoroquinazolin-4-yl)-3,8-diazabicyclo[3.2.1]octane-8-carboxylate (4 g, 9 mmol), Pd(PPh₃)₄ (946 mg, 1.8 mmol), CuI (624 mg), LiCl (1.03 g) in dioxane (60 mL) was stirred for 16 hours at 110° C. After completion, the resulting mixture was directly concentrated. Then the crude product was further purified by silica column (eluting with PE/EtOAc=3/2) to give the title product (1 g). MS (ESI, m/e) [M+1]⁺777.3.

### Step 4: tert-butyl-3-(7-(6-(bis(4-methoxybenzyl)amino)-4-chloro-3-iodopyridin-2-yl)-6-chloro-2,8-difluoroquinazolin-4-yl)-3,8-diazabicyclo[3.2.1]octane-8-carboxylate

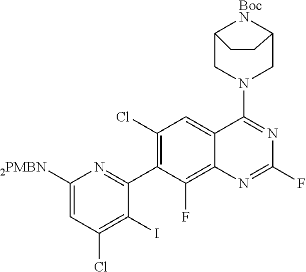

A mixture of tert-butyl-3-(7-(6-(bis(4-methoxybenzyl)amino)-4-chloropyridin-2-yl)-6-chloro-2,8-difluoroquinazolin-4-yl)-3,8-diazabicyclo[3.2.1]octane-8-carboxylate (430 mg, 0.6 mmol), Ag₂SO₄ (370 mg, 1.2 mmol), I₂ (310 mg, 1.2 mmol) in DMF (10 mL) was stirred for 1 hour at room temperature. After completion, the resulting mixture was directly poured into water and extracted with EtOAc. Then the crude product was further purified by silica column to give the title product (300 mg). MS (ESI, m/e) [M+1]⁺903.4

### Step 5: tert-butyl-3-(7-(6-(bis(4-methoxybenzyl)amino)-4-chloro-3-(trifluoromethyl)pyridin-2-yl)-6-chloro-2,8-difluoroquinazolin-4-yl)-3,8-diazabicyclo[3.2.1]octane-8-carboxylate

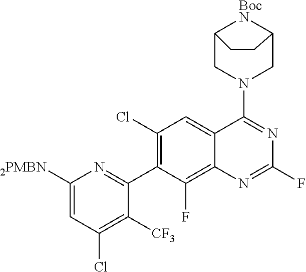

A mixture of tert-butyl-3-(7-(6-(bis(4-methoxybenzyl)amino)-4-chloro-3-iodopyridin-2-yl)-6-chloro-2,8-difluoroquinazolin-4-yl)-3,8-diazabicyclo[3.2.1]octane-8-carboxylate (90 mg, 0.1 mmol), CuI (95 mg, 0.5 mmol), methyl 2,2-difluoro-2-(fluorosulfonyl)acetate (192 mg, 1 mmol) in DMA (10 mL) was stirred for 2 hours at 90° C. After completion, the resulting mixture was directly poured into water and extracted with EtOAc. Then the crude product was further purified by silica column to give the title product (80 mg). MS (ESI, m/e) [M+1]⁺845.2

### Step 6: tert-butyl-3-(7-(6-(bis(4-methoxybenzyl)amino)-4-chloro-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

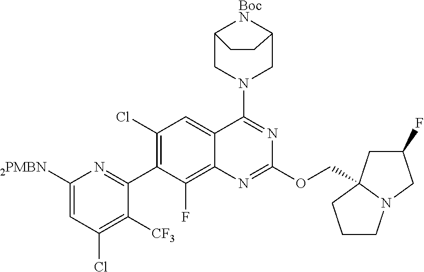

To a solution of ((2S,4R)-4-fluoro-1-methylpyrrolidin-2-yl)methanol (48 mg, 0.3 mmol) in THF (10 mL) was added NaH (11.2 mg) and the mixture was stirred for 30 mins at room temperature. Then, tert-butyl-3-(7-(6-(bis(4-methoxybenzyl)amino)-4-chloro-3-(trifluoromethyl)pyridin-2-yl)-6-chloro-2,8-difluoroquinazolin-4-yl)-3,8-diazabicyclo[3.2.1]octane-8-carboxylate (83 mg, 0.1 mmol) was added. The reaction mixture was stirred for 2 hours at room temperature. After completion, the reaction was quenched by H₂O. Solvents were evaporated and the crude product was purified by silica column (eluting with DCM/MeOH=10/1) to give the title product (27 mg). MS (ESI, m/e) [M+1]⁺984.4.

### Step 7: 6-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-4-chloro-5-(trifluoromethyl)pyridin-2-amine

A mixture of tert-butyl-3-(7-(6-(bis(4-methoxybenzyl)amino)-4-chloro-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (27 mg, 0.03 mmol) in TFA (4 mL) was stirred for 8 hours at 40° C. After completion, the resulting mixture was directly concentrated and the residue was adjusted to pH=9 with DIPEA. Then the residues were purified by C18 column (eluting with H₂O/CH₃CN=3/1) to afford the title product (2.4 mg, FA salt). ¹H NMR (500 MHz, CD₃OD) δ 8.51 (s, 1H), 7.87 (s, 1H), 6.85-6.82 (m, 1H), 5.31-5.46 (m, 1H), 4.58-4.36 (m, 4H), 3.85 (s, 2H), 3.76-3.64 (m, 2H), 3.59-3.39 (m, 3H), 3.24-3.10 (m, 1H), 2.49-2.18 (m, 3H), 2.18-1.87 (m, 7H). MS (ESI, m/e) [M+1]⁺644.4.

### Example 66: 4-(4-((3,8-diazabicyclo[3.2.1]octan-3-yl)-8-fluoro-2-(((2S,4R)-4-fluoro-1-methylpyrrolidin-)-yl)methoxy)-6-vinylquinazolin-7-yl)-7-fluorobenzo[d]thiazol-2-amine

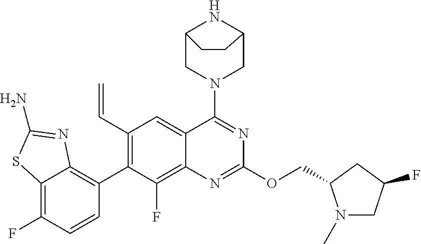 [SMILES: C=Cc1cc2c(N3CC4CCC(C3)N4)nc(OC[C@@H]3C[C@@H](F)CN3C)nc2c(F)c1-c1ccc(F)c2sc(N)nc12]

Synthetic Route

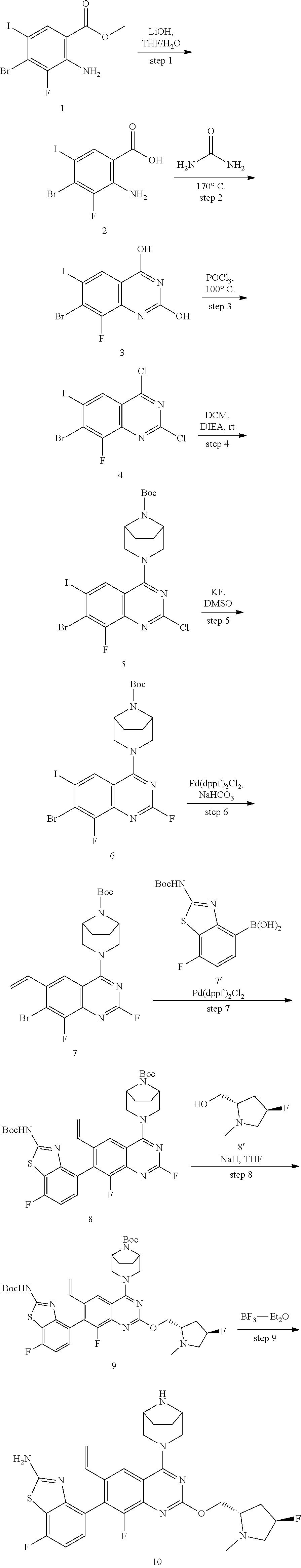

### Step 1: 2-amino-4-bromo-3-fluoro-5-iodobenzoic acid

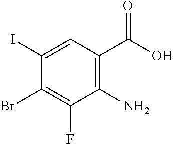 [SMILES: Nc1c(C(=O)O)cc(I)c(Br)c1F]

To a solution of methyl 2-amino-4-bromo-3-fluoro-5-iodobenzoate (1.87 g, 5.0 mmol) in tetrahydrofuran (8 mL) was added lithium hydroxide (8 mL, 1M in water). After the addition, the mixture was stirred at room temperature for 4 hrs. pH was adjusted to 3 with hydrogen chloride in water (2 N). The mixture was diluted with ethyl acetate and water. Combined organic layer was dried over sodium sulfate and evaporated to give the crude title compound (1.31 g) which was used directly in the next step. MS (ESI, m/e) [M+H]⁺ 359.6.

### Step 2: 7-bromo-8-fluoro-6-iodoquinazoline-2,4-diol

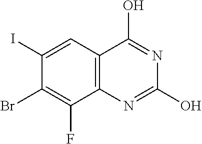 [SMILES: Oc1nc(O)c2cc(I)c(Br)c(F)c2n1]

2-amino-4-bromo-3-fluoro-5-iodobenzoic acid (1.30 g, 3.61 mmol) and urea (6.0 g, 100 mmol) was stirred at 170° C. for 7 hrs. Then, the mixture was cooled to 60° C. and hot water (60° C., 50 mL) was added and the mixture was stirred at room temperature for 0.5 h. Solid was filtered and washed with ethyl acetate for 6 times. Combined organic layer was dried over sodium sulfate and evaporate to give the crude title compound (900 mg) which was used directly in the next step. MS (ESI, m/e) [M+H]⁺ 384.5.

### Step 3: 7-bromo-2,4-dichloro-8-fluoro-6-iodoquinazoline

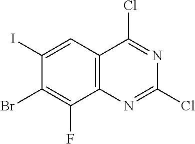 [SMILES: Fc1c(Br)c(I)cc2c(Cl)nc(Cl)nc12]

To a solution of 7-bromo-8-fluoro-6-iodoquinazoline-2,4-diol (900 mg, 234 mmol) in phosphorus oxychloride (10 mL) was added N,N-Diisopropylethylamine (2.13 g, 16.4 mmol) at room temperature. After addition, the mixture was stirred at 100° C. for 3 hrs. The mixture was cooled to room temperature and poured into ice water. The mixture was diluted with dichloromethane and water. Combined organic layer was dried over sodium sulfate and evaporated to give the title compound (880 mg) which was used directly in the next step. MS (ESI, m/e) [M+H]⁺ 420.4.

### Step 4: tert-butyl-3-(7-bromo-2-chloro-8-fluoro-6-iodoquinazolin-4-yl)-3,8-diazabicyclo[3.2.1]octane-8-carboxylate

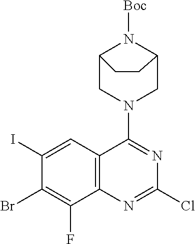 [SMILES: CC(C)(C)OC(=O)N1C2CCC1CN(c1nc(Cl)nc3c(F)c(Br)c(I)cc13)C2]

To a solution of 7-bromo-2,4-dichloro-8-fluoro-6-iodoquinazoline (800 mg, 2.01 mmol) in dichloromethane (10 mL) was added DIPEA (1 mL) and tert-butyl-3,8-diazabicyclo[3.2.1]octane-8-carboxylate (500 mg, 2.35 mmol) at 0° C. . After the addition, the mixture was stirred at 0° C. for 1 h. Then the mixture was diluted with dichloromethane and water. Combined organic layer was dried over sodium sulfate and evaporated. The residue was purified by column chromatography on silica to give the title compound (800 mg). MS (ESI, m/e) [M+H]⁺ 596.5.

### Step 5: tert-butyl-3-(7-bromo-2,8-difluoro-6-iodoquinazolin-4-yl)-3,8-diazabicyclo[3.2.1]octane-8-carboxylate

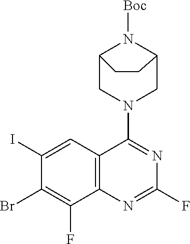 [SMILES: CC(C)(C)OC(=O)N1C2CCC1CN(c1nc(F)nc3c(F)c(Br)c(I)cc13)C2]

To a solution of tert-butyl-3-(7-bromo-2-chloro-8-fluoro-6-iodoquinazolin-4-yl)-3,8-diazabicyclo[3.2.1]octane-8-carboxylate (800 mg, 1.34 mmol) in dimethylsulfoxide (10 mL) was added potassium fluoride (580 mg, 10.0 mmol). After the addition, the mixture was stirred at 100° C. for 3 hrs. Then it was cooled to room temperature and diluted with dichloromethane and water. Combined organic layer was dried over sodium sulfate and evaporated. The residue was purified by column chromatography on silica to give the title compound (500 mg). MS (ESI, m/e) [M+H]⁺ 580.7.

### Step 6: tert-butyl-3-(7-bromo-2,8-difluoro-6-vinylquinazolin-4-yl)-3,8-diazabicyclo[3.2.1]octane-8-carboxylate

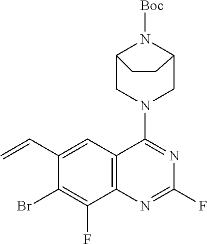 [SMILES: C=Cc1cc2c(N3CC4CCC(C3)N4C(=O)OC(C)(C)C)nc(F)nc2c(F)c1Br]

To a solution of tert-butyl-3-(7-bromo-2,8-difluoro-6-iodoquinazolin-4-yl)-3,8-diazabicyclo[3.2.1]octane-8-carboxylate (350 mg, 0.60 mmol) in dioxane:water (10 mL: 2 mL) was added potassium vinyltrifluoroborate (230 mg, 1.73 mmol), 1,1′-bis(diphenylphosphino)ferrocene-palladium(II)dichloride dichloromethane complex (50 mg, 0.06 mmol) and sodium bicarbonate (140 mg, 167 mmol) at room temperature. After the addition, the mixture was stirred at 75° C. for 4 hrs. Then, the mixture was cooled to room temperature, and diluted with dichloromethane and water. Combined organic layer was dried over sodium sulfate and evaporated. The residue was purified by column chromatography on silica to give the title compound (240 mg). MS (ESI, m/e) [M+H]⁺ 480.8

### Step 7: tert-butyl (3-(7-(2-((tert-butoxycarbonyl)amino)-7-fluorobenzo[d]thiazol-4-yl)-2,8-difluoro-6-vinylquinazolin-4-yl)-3,8-diazabicyclo[3.2.1]octane-8-carboxylate

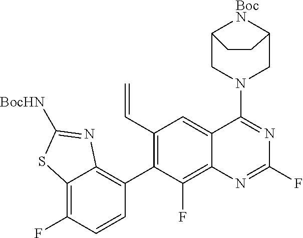 [SMILES: C=Cc1cc2c(N3CC4CCC(C3)N4C(=O)OC(C)(C)C)nc(F)nc2c(F)c1-c1ccc(F)c2sc(NC(=O)OC(C)(C)C)nc12]

To a solution of tert-butyl-3-(7-bromo-2,8-difluoro-6-vinylquinazolin-4-yl)-3,8-diazabicyclo[3.2.1]octane-8-carboxylate (240 mg, 0.50 mmol) in dioxane:water (15 mL: 3 mL) was added (2-((tert-butoxycarbonyl)amino)-7-fluorobenzo[d]thiazol-4-yl)boronic acid (190 mg, 0.61 mmol), 1,1′-bis(diphenylphosphino)ferrocene-palladium(II)dichloride (37 mg, 0.05 mmol) and sodium bicarbonate (124 mg, 148 mmol) at room temperature. After the addition, the mixture was stirred at 95° C. for 3 hrs. Then it was cooled to room temperature, and the mixture was diluted with dichloromethane and water. Combined organic layer was dried over sodium sulfate and evaporated. The residue was purified by column chromatography on silica to give the title compound (170 mg). MS (ESI, m/e) [M+H]⁺ 669.5.

### Step 8: tert-butyl-3-(7-(2-((tert-butoxycarbonyl)amino)-7-fluorobenzo[d]thiazol-4-yl)-8-fluoro-2-(((2S,4R)-4-fluoro-1-methylpyrrolidin-2-yl)methoxy)-6-vinylquinazolin-4-yl)-3,8-diazabicyclo[3.2.1]octane-8-carboxylate

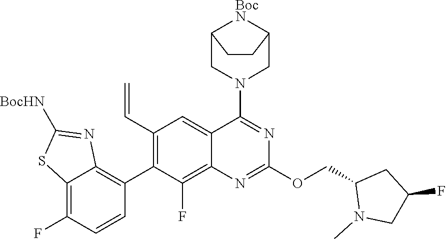 [SMILES: C=Cc1cc2c(N3CC4CCC(C3)N4C(=O)OC(C)(C)C)nc(OC[C@@H]3C[C@@H](F)CN3C)nc2c(F)c1-c1ccc(F)c2sc(NC(=O)OC(C)(C)C)nc12]

To a solution of ((2S,4R)-4-fluoro-1-methylpyrrolidin-2-yl)methanol (52 mg, 0.40 mmol) was add sodium hydride (60%) (16 mg, 0.40 mmol) at 0° C. and the mixture was stirred at room temperature for 0.5 h. Then, a solution of tert-butyl (3-(7-(2-((tert-butoxycarbonyl)amino)-7-fluorobenzo[d]thiazol-4-yl)-2,8-difluoro-6-vinylquinazolin-4-yl)-3,8-diazabicyclo[3.2.1]octane-8-carboxylate (70 mg, 0.10 mmol) in tetrahydrofuran (1 mL) was added dropwise. The mixture was stirred at room temperature for another 1 h and then quenched with aq ammonium chloride at 0° C. The mixture was diluted with dichloromethane and water and the organic layer was combined, dried over sodium sulfate and evaporated. The residue was purified by preparative thin layer chromatography on silica to give the title compound (30 mg). MS (ESI, m/e) [M+H]⁺ 782.7.

### Step 9: 4-(4-((3,8-diazabicyclo[3.2.1]octan-3-yl)-8-fluoro-2-(((2S,4R)-4-fluoro-1-methylpyrrolidin-2-yl)methoxy)-6-vinylquinazolin-7-yl)-7-fluorobenzo[d]thiazol-2-amine

To a solution of tert-butyl-3-(7-(2-((tert-butoxycarbonyl)amino)-7-fluorobenzo[d]thiazol-4-yl)-8-fluoro-2-(((2S,4R)-4-fluoro-1-methylpyrrolidin-2-yl)methoxy)-6-vinylquinazolin-4-yl)-3,8-diazabicyclo[3.2.1]octane-8-carboxylate (30 mg, 0.038 mmol) in dichloromethane (8 mL) was added boron trifluoride etherate (0.1 mL) at room temperature. After the addition, the mixture was stirred at room temperature for 1.5 hrs. Then, the mixture was quenched with methanol and then solvents were evaporated. The residue was purified by Prep-HPLC to give the title compound (10 mg). ¹H NMR (500 MHz, CD₃OD) δ 7.96 (s, 1H), 7.17-7.10 (m, 1H), 7.01-6.94 (m, 1H), 6.46-6.36 (m, 1H), 5.72-5.63 (m, 1H), 5.20-5.09 (m, 2H), 4.61-4.42 (m, 4H), 3.83-3.61 (m, 4H), 3.55-3.43 (m, 1H), 3.26-3.17 (m, 1H), 2.75-2.62 (m, 1H), 2.57 (s, 3H), 2.37-2.22 (m, 1H), 2.07-1.79 (m, 5H). MS (ESI, m/e) [M+H]⁺ 582.4.

### Example 67: 4-(4-(3,8-diazabicyclo[3.2.1]octan-3-yl)-6-ethyl-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine

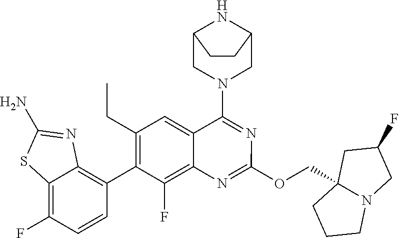 [SMILES: CCc1cc2c(N3CC4CCC(C3)N4)nc(OC[C@@]34CCCN3C[C@H](F)C4)nc2c(F)c1-c1ccc(F)c2sc(N)nc12]

Synthetic Route

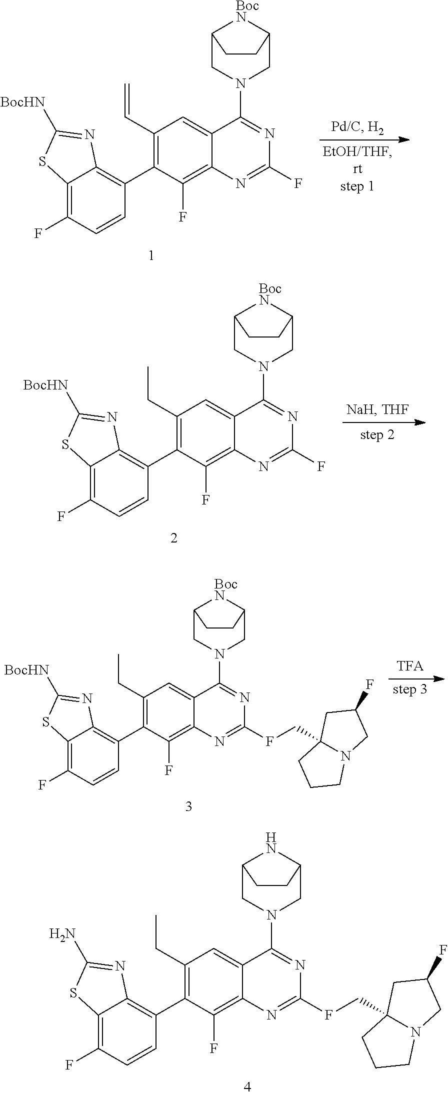

### Step 1: tert-buty-3-(7-(2-((tert-butoxycarbonyl)amino)-7-fluorobenzo[d]thiazol-4-yl)-6-ethyl-2,8-difluoroquinazolin-4-yl)-3,8-diazabicyclo[3.2.1]octane-8-carboxylate

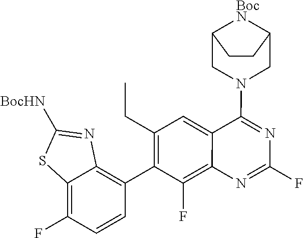 [SMILES: CCc1cc2c(N3CC4CCC(C3)N4C(=O)OC(C)(C)C)nc(F)nc2c(F)c1-c1ccc(F)c2sc(NC(=O)OC(C)(C)C)nc12]

To a solution of tert-butyl-3-(7-(2-((tert-butoxycarbonyl)amino)-7-fluorobenzo[d]thiazol-4-yl)-2,8-difluoro-6-vinylquinazolin-4-yl)-3,8-diazabicyclo[3.2.1]octane-8-carboxylate (60 mg, 0.09 mmol) in ethanol/tetrahydrofuran (2 mL/6 mL) was added Palladium on carbon (30 mg), and the mixture was stirred at room temperature for 4 hrs. Solid was filtered off, and the filtrate was evaporated to give the crude title compound (50 mg) which was used directly in the next step. MS (ESI, m/e) [M+H]⁺ 671.5.

### Step 2: tert-butyl-3-(7-(2-((tert-butoxycarbonyl)amino)-7-fluorobenzo[d]thiazol-4-yl)-6-ethyl-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

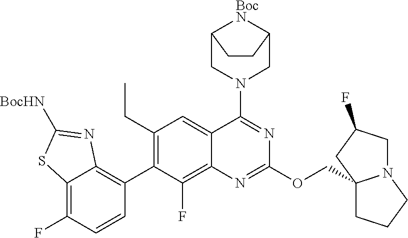 [SMILES: CCc1cc2c(N3CC4CCC(C3)N4C(=O)OC(C)(C)C)nc(OC[C@@]34CCCN3C[C@H](F)C4)nc2c(F)c1-c1ccc(F)c2sc(NC(=O)OC(C)(C)C)nc12]

To a solution of ((2R,7aS)-2-fluorotetrahydro-1H-pyrrolizin-7a(5H)-yl)methanol (40 mg, 0.25 mmol) was add sodium hydride (60%) (11 mg, 0.27 mmol) at 0° C. and the mixture was stirred at room temperature for 0.5 h. Then, a solution of tert-buty-3-(7-(2-((tert-butoxycarbonyl)amino)-7-fluorobenzo[d]thiazol-4-yl)-6-ethyl-2,8-difluoroquinazolin-4-yl)-3,8-diazabicyclo[3.2.1]octane-8-carboxylate (50 mg, 0.07 mmol) in tetrahydrofuran (1 mL) was added dropwise. The mixture was stirred at room temperature for another 1 h and then quenched with aq ammonium chloride at 0° C. The mixture was diluted with dichloromethane and water and the organic layer was combined, dried over sodium sulfate and evaporated. The residue was purified by preparative thin layer chromatography on silica to give the title compound (30 mg). MS (ESI, m/e) [M+H]⁺ 810.7.

### Step 3: 4-(4-(3,8-diazabicyclo[3.2.1]octan-3-yl)-6-ethyl-8-fluoro-2-(((2R,7aS)-2-fluorotetrahydro-1H-pyrrolizin-7a(5H)-yl)methoxy)quinazolin-7-yl)-7-fluorobenzo[d]thiazol-2-amine

To a solution of tert-butyl-3-(7-(2-((tert-butoxycarbonyl)amino)-7-fluorobenzo[d]thiazol-4-yl)-6-ethyl-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (30 mg, 0.037 mmol) in dichloromethane (2 mL) was added trifluoroacetic acid (1.5 mL) at room temperature. After the addition, the mixture was stirred at room temperature for 2.5 hrs. Then the solution was evaporated, and the residue was adjusted to pH=10 with aq sodium carbonate. The mixture was mixed with dichloromethane:methanol=10:1, and the organic layer was dried over sodium sulfate and evaporated. The residue was lyophilized to give the title compound (10 mg). ¹H NMR (500 MHz, CD₃OD) δ 7.60 (s, 1H), 7.19-7.12 (m, 1H), 7.01-6.94 (m, 1H), 5.39-5.20 (m, 2H), 4.56-4.40 (m, 2H), 4.31-4.15 (m, 2H), 3.68-3.54 (m, 4H), 3.26-3.12 (m, 3H), 3.05-2.94 (m, 2H), 2.75-2.62 (m, 1H), 2.57-2.41 (m, 2H), 2.37-2.07 (m, 3H), 1.90-1.78 (m, 4H), 1.05-1.02 (m, 3H). MS (ESI, m/e) [M+H]⁺ 610.4.

### Example 68: 4-(4-(3,8-diazabicyclo[3.2.1]octan-3-yl)-6-chloro-2-((1-((dimethylamino)-methyl)cyclobutyl)methoxy)-8-fluoroquinazolin-7-yl)-7-fluorobenzo[d]thiazol-2-amine

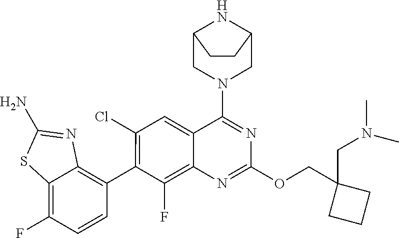 [SMILES: CN(C)CC1(COc2nc(N3CC4CCC(C3)N4)c3cc(Cl)c(-c4ccc(F)c5sc(N)nc45)c(F)c3n2)CCC1]

Synthetic Route

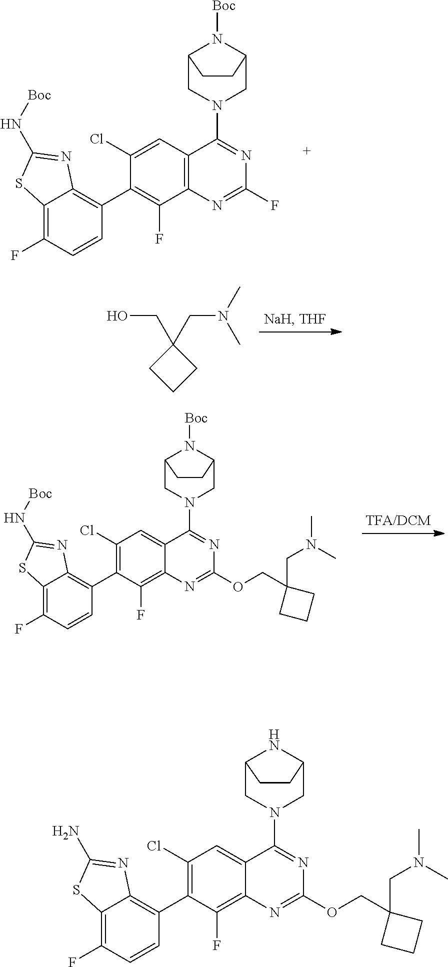

### Step 1: tert-butyl 3-(7-(2-((tert-butoxycarbonyl)amino)-7-fluorobenzo[d]thiazol-4-yl)-6-chloro-2-((1-((dimethylamino)methyl)cyclobutyl)methoxy)-8-fluoroquinazolin-4-yl)-3,8-diazabicyclo[3.2.1]octane-8-carboxylate

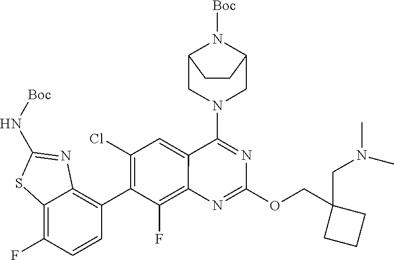 [SMILES: CN(C)CC1(COc2nc(N3CC4CCC(C3)N4C(=O)OC(C)(C)C)c3cc(Cl)c(-c4ccc(F)c5sc(NC(=O)OC(C)(C)C)nc45)c(F)c3n2)CCC1]

To a solution of (1-((dimethylamino)methyl)cyclobutyl)methanol (64 mg, 0.446 mmol) in THF (3 mL) was added NaH (40 mg, 1.0 mmol), and the mixture was stirred at room temperature for 0.5 hour. Then, a solution of tert-butyl 3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (200 mg, 0.296 mmol) in THF (1.0 mL) was added, and the mixture was stirred for more 4 hours at room temperature. The reaction mixture was diluted with EtOAc and water, extracted with EtOAc twice, and the combined organic layer was dried over sodium sulfate and evaporated. The residue was purified by chromatography column on silica (eluting with MeOH/DCM=5-7%) to give the title product (150 mg). MS (ESI, m/e) [M+1]⁺801.

### Step 2: 4-(4-(3,8-diazabicyclo[3.2.1]octan-3-yl)-6-chloro-2-((1-((dimethylamino)methyl)cyclobutyl)methoxy)-8-fluoroquinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-6-chloro-2-((1-((dimethylamino)methyl)cyclobutyl)methoxy)-8-fluoroquinazolin-4-yl)-3,8-diazabicyclo[3.2.1]octane-8-carboxylate (150 mg, 0.188 mmol) in DCM (8 mL) was added TFA (2 mL) and the mixture was stirred for 4 hours at room temperature. Then, solvent was evaporated to give the crude product, which was dissolved with DCM (20 mL)/H₂O (10 mL), and the pH was adjusted to 9 and extracted with DCM/MeOH (v=10/1) twice. Combined organic layer was concentrated and the residue was purified by chromatography column on silica (eluting with MeOH/DCM=20%) to give the title product (64.9 mg). ¹H NMR (500 MHz, MeOD) δ 7.86 (s, 1H), 7.21 (dd, J=8.4, 5.5 Hz, 1H), 6.98 (t, J=8.8 Hz, 1H), 4.61-4.40 (m, 4H), 3.70-3.54 (m, 4H), 2.58 (s, 2H), 2.22 (s, 6H), 2.10-1.88 (m, 6H), 1.87-1.77 (m, 4H). MS (ESI, m/e) [M+H]⁺ 600.4.

### Example 69: 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(prop-1-en-2-yl)quinazolin-7-yl)-7-fluorobenzo[d]thiazol-2-amine

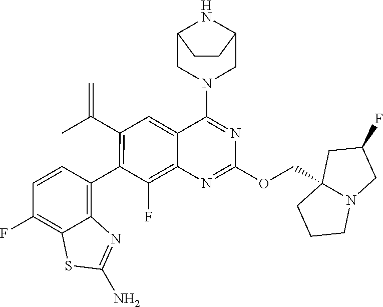 [SMILES: C=C(C)c1cc2c(N3CC4CCC(C3)N4)nc(OC[C@@]34CCCN3C[C@H](F)C4)nc2c(F)c1-c1ccc(F)c2sc(N)nc12]

Synthetic Route

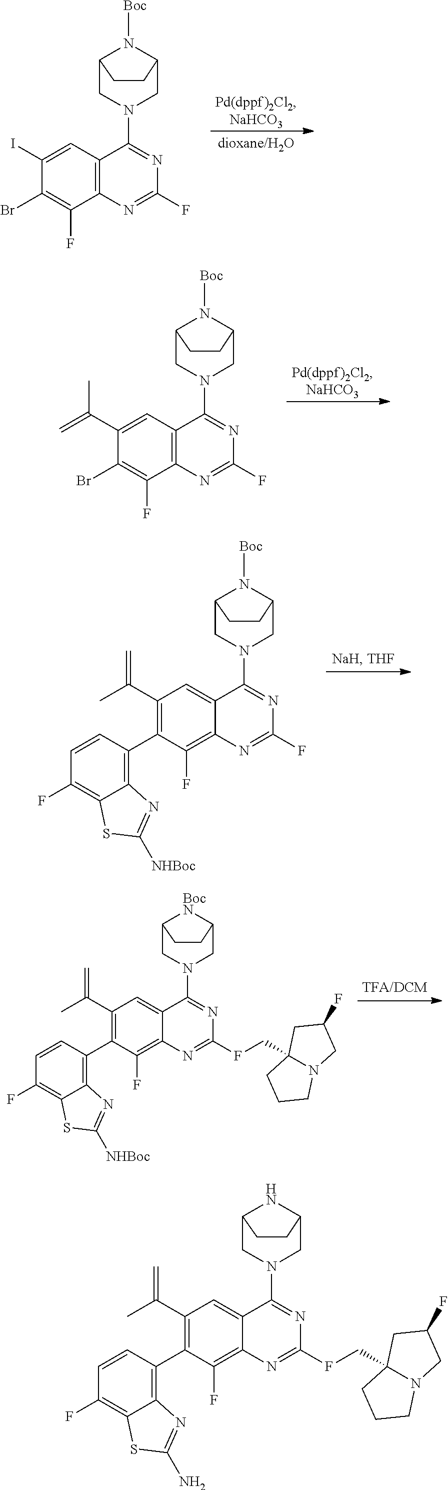

### Step 1: tert-butyl 3-(7-bromo-2,8-difluoro-6-(prop-1-en-2-yl)quinazolin-4-yl)-3,8-diazabicyclo[3.2.1]octane-8-carboxylate

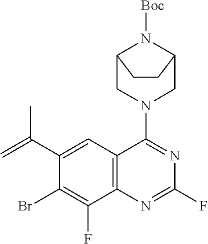 [SMILES: C=C(C)c1cc2c(N3CC4CCC(C3)N4C(=O)OC(C)(C)C)nc(F)nc2c(F)c1Br]

A mixture of tert-butyl 3-(7-bromo-2,8-difluoro-6-iodoquinazolin-4-yl)-3,8-diazabicyclo[3.2.1]octane-8-carboxylate (300 mg, 0.63 mmol), Potassium isopropenyltrifluoroborate (270 mg, 1.82 mmol), Pd(dppf)Cl₂ (50 mg, 0.07 mmol), dioxane and NaHCO₃ (50 mg, 0.07 mmol) in H₂O (2.0 mL) was stirred at 75° C. for 4 hours. The resulting mixture was concentrated. The residue was diluted with EtOAc and water, extracted with EtOAc twice, and the organic layer was combined, dried over sodium sulfate and evaporated. The residue was purified by chromatography column on silica (eluting with MeOH/DCM=5-7%) to give the title product (160 mg). MS (ESI, m/e) [M+1]⁺496.4.

### Step 2: tert-butyl 3-(7-(2-((tert-butoxycarbonyl)amino)-7-fluorobenzo[d]thiazol-4-yl)-2,8-difluoro-6-(prop-1-en-2-yl)quinazolin-4-yl)-3,8-diazabicyclo[3.2.1]octane-8-carboxylate

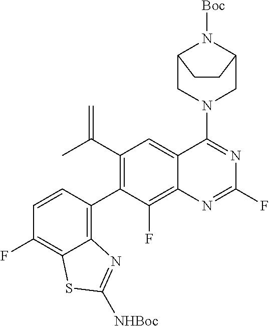 [SMILES: C=C(C)c1cc2c(N3CC4CCC(C3)N4C(=O)OC(C)(C)C)nc(F)nc2c(F)c1-c1ccc(F)c2sc(NC(=O)OC(C)(C)C)nc12]

A mixture of tert-butyl 3-(7-bromo-2,8-difluoro-6-(prop-1-en-2-yl)quinazolin-4-yl)-3,8-diazabicyclo[3.2.1]octane-8-carboxylate (200 mg, 0.40 mmol), (2-((tert-butoxycarbonyl)amino)-7-fluorobenzo[d]thiazol-4-yl)boronic acid (250 mg, 0.80 mmol), Pd(dppf)Cl₂ (33 mg, 0.045 mmol), dioxane and NaHCO₃ (100 mg, 1.20 mmol) in H₂O (2.0 mL) was stirred at 95° C. for 4 hours. The resulting mixture was concentrated. The residue was diluted with EtOAc and water, extracted with EtOAc twice, and the organic layer was combined, dried over sodium sulfate and evaporated. The residue was purified by chromatography column on silica (eluting with EA/PE=20-30%) to give the title product (120 mg). MS (ESI, m/e) [M+1]⁺683.3.

### Step 3: tert-butyl 3-(7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-(prop-1-en-2-yl)quinazolin-4-yl)-3,8-diazabicyclo[3.2.1]octane-8-carboxylate

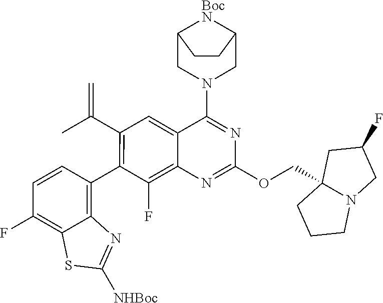 [SMILES: C=C(C)c1cc2c(N3CC4CCC(C3)N4C(=O)OC(C)(C)C)nc(OC[C@@]34CCCN3C[C@H](F)C4)nc2c(F)c1-c1ccc(F)c2sc(NC(=O)OC(C)(C)C)nc12]

To a solution of ((2R,7aS)-2-fluorotetrahydro-1H-pyrrolizin-7a(5H)-yl)methanol (50 mg, 0.314 mmol) in THF (3 mL) was added NaH (15 mg, 0.375 mmol), and the mixture was stirred at room temperature for 0.5 hour, then added tert-butyl 3-(7-(2-((tert-butoxycarbonyl)amino)-7-fluorobenzo[d]thiazol-4-yl)-2,8-difluoro-6-(prop-1-en-2-yl)quinazolin-4-yl)-3,8-diazabicyclo[3.2.1]octane-8-carboxylate (100 mg, 0.146 mmol) in THF (1.0 mL), then the mixture was stirred for 4 hours at room temperature. The reaction mixture was diluted with EtOAc and water, extracted with EtOAc twice, and the organic layer was combined, dried over sodium sulfate and evaporated. The residue was purified by chromatography column on silica (eluting with MeOH/DCM=5-7%) to give the title product (50 mg). MS (ESI, m/e) [M+1]⁺822.3

### Step 4: 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(prop-1-en-2-yl)quinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-(prop-1-en-2-yl)quinazolin-4-yl)-3,8-diazabicyclo[3.2.1]octane-8-carboxylate (30 mg, 0.036 mmol) in DCM (6 mL) was added TFA (1.5 mL ) and the mixture was stirred for 4 hours at room temperature. Then, the resulting mixture was evaporated at room temperature to give the crude product, which was dissolved with DCM (20 mL)/H₂O (10 mL), and the pH was adjusted to 9 and extracted with DCM/MeOH (v=10/1) twice, and the organic layer was combined, the residue was purified by chromatography column on silica (eluting with MeOH/DCM=20%) to give the title product (2.9 mg). ¹H NMR (500 MHz, CD₃OD) δ 7.60 (d, J=6.5 Hz, 1H), 7.18-7.08 (m, 1H), 6.92 (q, J=9.0 Hz, 1H), 5.30 (d, J=53.9 Hz, 1H), 4.97 (s, 1H), 4.88 (s, 1H), 4.57-4.41 (m, 2H), 4.30-4.17 (m, 2H), 3.67-3.50 (m, 4H), 3.26-2.89 (m, 4H), 2.39-2.09 (m, 3H), 2.04-1.95 (m, 2H), 1.93-1.74 (m, 5H), 1.65 (s, 3H), 1.43 (s, 1H). MS (ESI, m/e) [M+H]⁺ 622.5.

### Example 70: 4-(4-(3,8-diazabicyclo[3.2.1]octan-3-yl)-6-chloro-2-((1-((dimethylamino)methyl)cyclopropyl)methoxy)-8-fluoroquinazolin-7-yl)-7-fluorobenzo[d]thiazol-2-amine

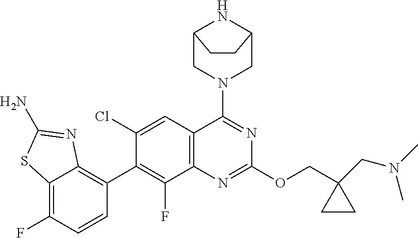 [SMILES: CN(C)CC1(COc2nc(N3CC4CCC(C3)N4)c3cc(Cl)c(-c4ccc(F)c5sc(N)nc45)c(F)c3n2)CC1]

Synthetic Route

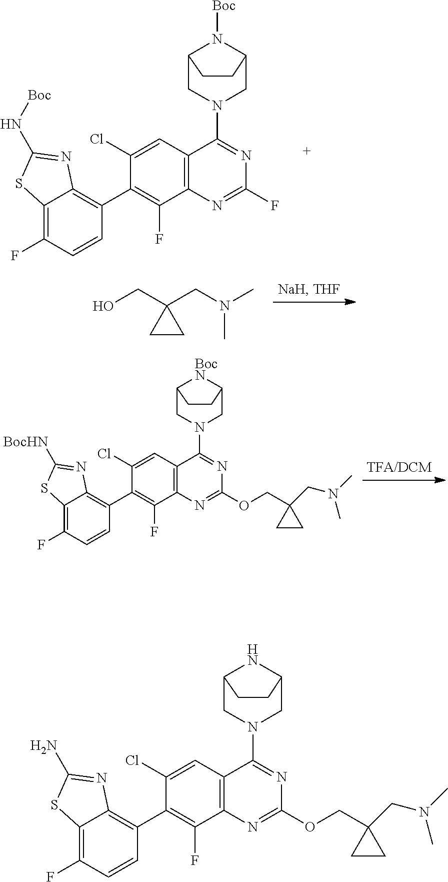

### Step 1: tert-butyl 3-(7-(2-((tert-butoxycarbonyl)amino)-7-fluorobenzo[d]thiazol-4-yl)-6-chloro-2-((1-((dimethylamino)methyl)cyclopropyl)methoxy)-8-fluoroquinazolin-4-yl)-3,8-diazabicyclo[3.2.1]octane-8-carboxylate

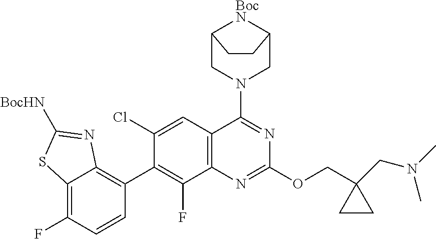 [SMILES: CN(C)CC1(COc2nc(N3CC4CCC(C3)N4C(=O)OC(C)(C)C)c3cc(Cl)c(-c4ccc(F)c5sc(NC(=O)OC(C)(C)C)nc45)c(F)c3n2)CC1]

To a solution of (1-((dimethylamino)methyl)cyclopropyl)methanol (17 mg, 0.134 mmol) in THF (3 mL) was added NaH (11 mg, 0.267 mmol), and the mixture was stirred at room temperature for 0.5 hour, then a solution of tert-butyl-3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (60 mg, 0.089 mmol) in THF (1.0 mL) was added. The mixture was stirred for 4 hours at room temperature. The reaction mixture was diluted with EtOAc and water, extracted with EtOAc twice, and the organic layer was combined, dried over sodium sulfate and evaporated. The residue was purified by chromatography column on silica (eluting with MeOH/DCM=5-10%) to give the title product (40 mg). MS (ESI, m/e) [M+1]⁺787.

### Step 2: 4-(4-(3,8-diazabicyclo[3.2.1]octan-3-yl)-6-chloro-2-((1-((dimethylamino)methyl)cyclopropyl)methoxy)-8-fluoroquinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-6-chloro-2-((1-((dimethylamino)methyl)cyclopropyl)methoxy)-8-fluoroquinazolin-4-yl)-3,8-diazabicyclo[3.2.1]octane-8-carboxylate (40 mg, 0.051 mmol) in DCM (6 mL) was added TFA (1.5 mL) and the mixture was stirred for 4 hours at room temperature. Then, the resulting mixture was evaporated at room temperature to give the crude product, This crude product was dissolved with DCM (20 mL)/H₂O (10 mL), and the pH was adjusted to 9 and extracted with DCM/MeOH (v=10/1) twice, and the organic layer was combined, the residue was purified by chromatography column on silica (eluting with MeOH/DCM=20%) to give the title product (24.7 mg). ¹H NMR (500 MHz, CD₃OD) δ 7.85 (d, J=1.2 Hz, 1H), 7.21 (td, J=8.7, 5.5 Hz, 1H), 7.01-6.93 (m, 1H), 4.57-4.27 (m, 4H), 3.72-3.52 (m, 4H), 2.46 (s, 2H), 2.31 (s, 6H), 1.88-1.74 (m, 4H), 0.71 (t, J=5.1 Hz, 2H), 0.52 (t, J=5.1 Hz, 2H), MS (ESI, m/e) [M+H]⁺ 586.3.

### Example 71: 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-isopropylquinazolin-7-yl)-7-fluorobenzo[d]thiazol-2-amine

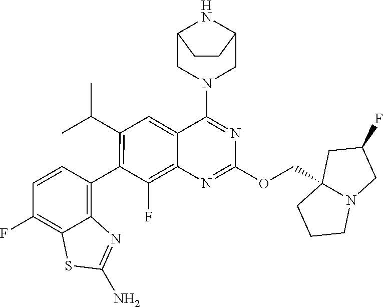 [SMILES: CC(C)c1cc2c(N3CC4CCC(C3)N4)nc(OC[C@@]34CCCN3C[C@H](F)C4)nc2c(F)c1-c1ccc(F)c2sc(N)nc12]

Synthetic Route

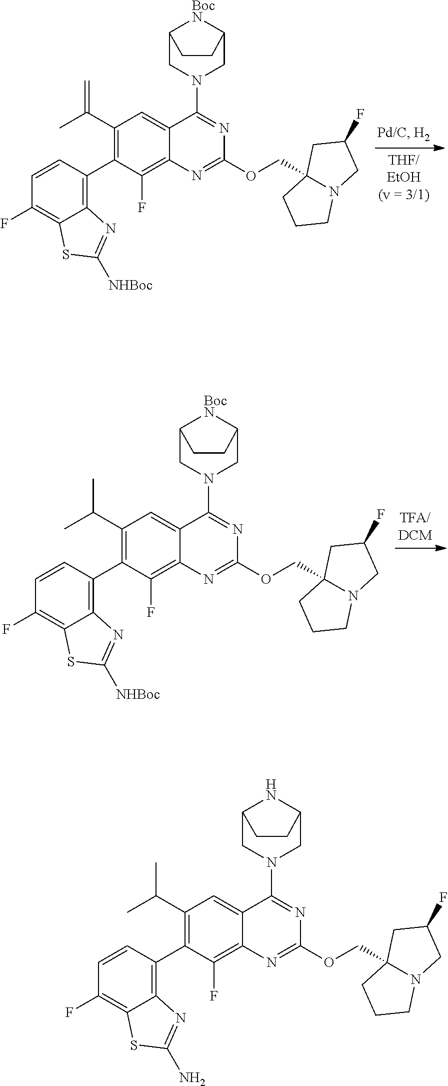 [SMILES: C=C(C)c1cc2c(N3CC4CCC(C3)N4C(=O)OC(C)(C)C)nc(OC[C@@]34CCCN3C[C@H](F)C4)nc2c(F)c1-c1ccc(F)c2sc(NC(=O)OC(C)(C)C)nc12 ; CC(C)c1cc2c(N3CC4CCC(C3)N4C(=O)OC(C)(C)C)nc(OC[C@@]34CCCN3C[C@H](F)C4)nc2c(F)c1-c1ccc(F)c2sc(NC(=O)OC(C)(C)C)nc12 ; CC(C)c1cc2c(N3CC4CCC(C3)N4)nc(OC[C@@]34CCCN3C[C@H](F)C4)nc2c(F)c1-c1ccc(F)c2sc(N)nc12]

### Step 1: tert-butyl 3-(7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-isopropylquinazolin-4-yl)-3,8-diazabicyclo[3.2.1]octane-8-carboxylate

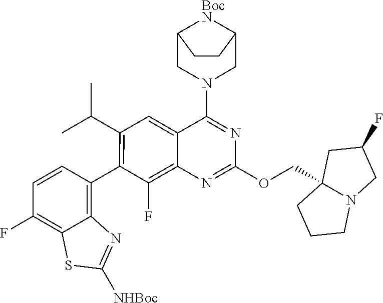 [SMILES: CC(C)c1cc2c(N3CC4CCC(C3)N4C(=O)OC(C)(C)C)nc(OC[C@@]34CCCN3C[C@H](F)C4)nc2c(F)c1-c1ccc(F)c2sc(NC(=O)OC(C)(C)C)nc12]

To a solution of tert-butyl 3-(7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-(prop-1-en-2-yl)quinazolin-4-yl)-3,8-diazabicyclo[3.2.1]octane-8-carboxylate (100 mg, 0.12 mmol) in THF (6 mL) and ethanol (2 mL) was added 10% Pd/C, (wet, 20 mg), and the mixture was stirred at room temperature for 24 hours under hydrogen atmosphere. Then, the mixture was filtered and the filtrate was evaporated to give the title product (70 mg). MS (ESI, m/e) [M+H]⁺ 824.3.

### Step 2: 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-isopropylquinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-isopropylquinazolin-4-yl)-3,8-diazabicyclo[3.2.1]octane-8-carboxylate (70 mg, 0.085 mmol) in DCM (6 mL) was added TFA (1.5 mL ) and the mixture was stirred for 4 hours at room temperature. Then, the resulting mixture was evaporated at room temperature to give the crude product, which was dissolved with DCM (20 mL)/H₂O (10 mL), and the pH was adjusted to 9 and extracted with DCM/MeOH (v=10/1) twice. Combined organic layer was concentrated and the residue was purified by chromatography column on silica (eluting with MeOH/DCM=20%) to give the title product (45 mg). ¹H NMR (500 MHz, MeOD) δ 7.67 (s, 1H), 7.20-7.13 (m, 1H), 7.00 (t, J=8.8 Hz, 1H), 5.50 (d, J=52.4 Hz, 1H), 4.72-4.52 (m, 4H), 4.13 (s, 2H), 3.92-3.65 (m, 5H), 3.38 (d, J=5.9 Hz, 1H), 2.83 (dt, J=13.4, 6.6 Hz, 1H), 2.66-2.22 (m, 5H), 2.15-2.02 (m, 5H), 1.16 (d, J=6.8 Hz, 6H). MS (ESI, m/e) [M+H]⁺ 624.4.

### Example 72: 4-(4-(3,8-diazabicyclo[3.2.1]octan-3-yl)-8-fluoro-2-(((2S)-1-(4-fluoro-2,2-dimethylpiperidin-1-yl)propan-2-yl)oxy)-6-(trifluoromethyl)quinazolin-7-yl)-7-fluorobenzo[d]thiazol-2-amine

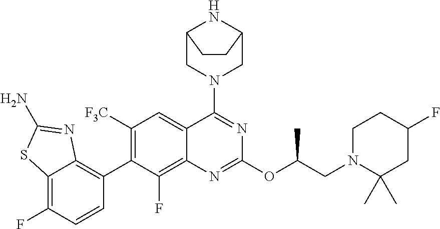 [SMILES: C[C@@H](CN1CCC(F)CC1(C)C)Oc1nc(N2CC3CCC(C2)N3)c2cc(C(F)(F)F)c(-c3ccc(F)c4sc(N)nc34)c(F)c2n1]

Synthetic Route

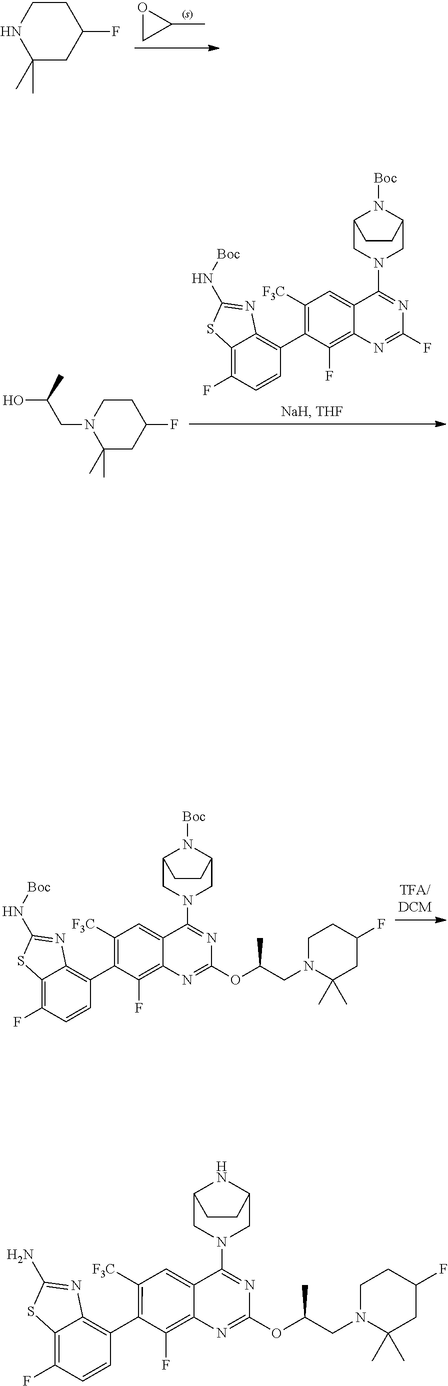

### Step 1: (2S)-1-(4-fluoro-2,2-dimethylpiperidin-1-yl)propan-2-ol

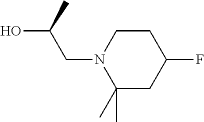

To a mixture of (S)-2-methyloxirane (60 mg, 1.03 mmol) in H₂O (5 mL) was added 4-fluoro-2,2-dimethylpiperidine (130 mg, 0.992 mmol), The mixture was stirred for 16 hours at room temperature. The reaction mixture was extracted with DCM/MeOH (v=10/1), and the organic layer was combined, dried over sodium sulfate and evaporated to give the crude product (100 mg) used directly in next step. MS (ESI, m/e) [M+1]⁺190.

### Step 2: tert-butyl 3-(7-(2-((tert-butoxycarbonyl)amino)-7-fluorobenzo[d]thiazol-4-yl)-8-fluoro-2-(((2S)-1-(4-fluoro-2,2-dimethylpiperidin-1-yl)propan-2-yl)oxy)-6-(trifluoromethyl)quinazolin-4-yl)-3,8-diazabicyclo[3.2.1]octane-8-carboxylate

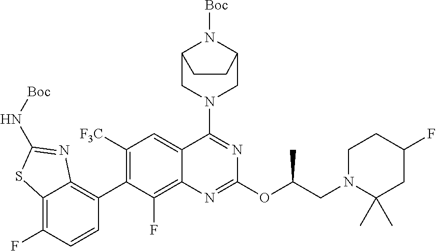 [SMILES: C[C@@H](CN1CCC(F)CC1(C)C)Oc1nc(N2CC3CCC(C2)N3C(=O)OC(C)(C)C)c2cc(C(F)(F)F)c(-c3ccc(F)c4sc(NC(=O)OC(C)(C)C)nc34)c(F)c2n1]

To a solution of (2S)-1-(4-fluoro-2,2-dimethylpiperidin-1-yl)propan-2-ol (50 mg, 0.26 mmol) in THF (3 mL) was added NaH (10 mg, 0.25 mmol), and the mixture was stirred at room temperature for 0.5 hour, then a solution of tert-butyl 3-(7-(2-((tert-butoxycarbonyl)amino)-7-fluorobenzo[d]thiazol-4-yl)-2,8-difluoro-6-(trifluoromethyl)quinazolin-4-yl)-3,8-diazabicyclo[3.2.1]octane-8-carboxylate (50 mg, 0.07 mmol) in THF (0.5 mL) was added. The mixture was stirred for 4 hours at room temperature. The reaction mixture was diluted with EtOAc and water, extracted with EtOAc twice, and the organic layer was combined, dried over sodium sulfate and evaporated. The residue was purified by chromatography column on silica (eluting with MeOH/DCM=5-7%) to give the title product (30 mg). MS (ESI, m/e) [M+1]⁺880.

### Step 3: 4-(4-(3,8-diazabicyclo[3.2.1]octan-3-yl)-8-fluoro-2-(((2S)-1-(4-fluoro-2,2-dimethylpiperidin-1-yl)propan-2-yl)oxy)-6-(trifluoromethyl)quinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-8-fluoro-2-(((2S)-1-(4-fluoro-2,2-dimethylpiperidin-1-yl)propan-2-yl)oxy)-6-(trifluoromethyl)quinazolin-4-yl)-3,8-diazabicyclo[3.2.1]octane-8-carboxylate (30 mg, 0.034 mmol) in DCM (6 mL) was added TFA (1.5 mL) and stirred for 4 hours at room temperature. Then the resulting mixture was evaporated at room temperature to give the crude product, which was dissolved with DCM (20 mL)/H2O (10 mL), and the pH was adjusted to 9 and extracted with DCM/MeOH (v=10/1) twice, and the organic layer was combined, the residue was purified by Prep-HPLC to give the title product (14.7 mg). ¹H NMR (500 MHz, CD₃OD) δ 8.52 (s, 1H), 8.12 (d, J=8.9 Hz, 1H), 7.18 (dd, J=8.2, 4.3 Hz, 1H), 6.96 (t, J=8.8 Hz, 1H), 5.69-5.38 (m, 2H), 4.78-4.44 (m, 2H), 4.00-3.67 (m, 4H), 3.21-2.45 (m, 3H), 2.24-2.10 (m, 1H), 2.06-1.83 (m, 4H), 1.80-1.57 (m, 1H), 1.54-1.37 (m, 3H), 1.35-1.16 (m, 4H), 1.12-0.97 (m, 2H). MS (ESI, m/e) [M+H]⁺ 680.4.

### Example 73: 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-methylbenzo[d]thiazol-2-amine

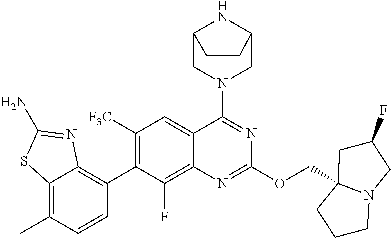 [SMILES: Cc1ccc(-c2c(C(F)(F)F)cc3c(N4CC5CCC(C4)N5)nc(OC[C@@]45CCCN4C[C@H](F)C5)nc3c2F)c2nc(N)sc12]

Synthetic Route

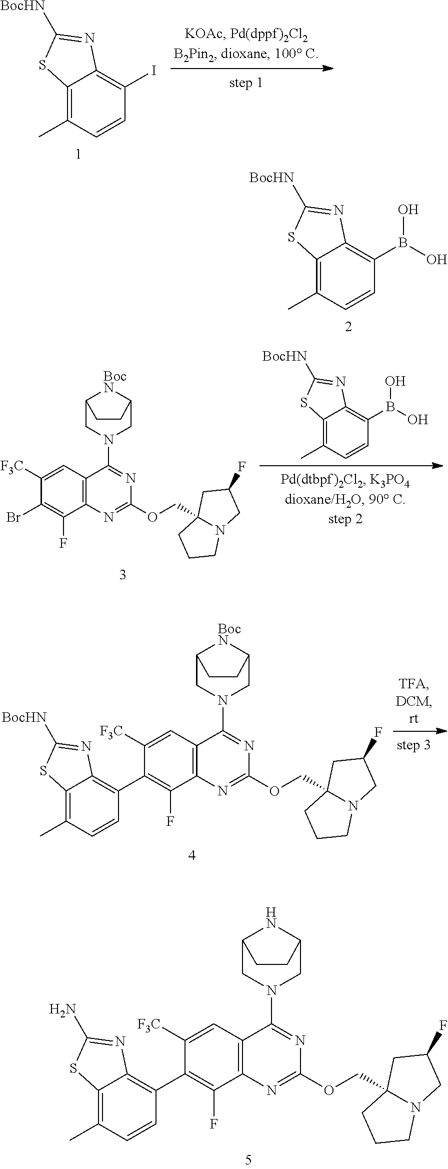 [SMILES: Cc1ccc(I)c2nc(NC(=O)OC(C)(C)C)sc12 ; Cc1ccc(B(O)O)c2nc(NC(=O)OC(C)(C)C)sc12 ; CC(C)(C)OC(=O)N1C2CCC1CN(c1nc(OC[C@@]34CCCN3C[C@H](F)C4)nc3c(F)c(Br)c(C(F)(F)F)cc13)C2 ; Cc1ccc(B(O)O)c2nc(NC(=O)OC(C)(C)C)sc12 ; Cc1ccc(-c2c(C(F)(F)F)cc3c(N4CC5CCC(C4)N5C(=O)OC(C)(C)C)nc(OC[C@@]45CCCN4C[C@H](F)C5)nc3c2F)c2nc(NC(=O)OC(C)(C)C)sc12 ; Cc1ccc(-c2c(C(F)(F)F)cc3c(N4CC5CCC(C4)N5)nc(OC[C@@]45CCCN4C[C@H](F)C5)nc3c2F)c2nc(N)sc12]

### Step 1: (2-((tert-butoxycarbonyl)amino)-7-methylbenzo[d]thiazol-4-yl)boronic acid

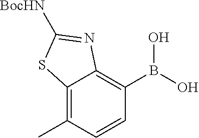 [SMILES: Cc1ccc(B(O)O)c2nc(NC(=O)OC(C)(C)C)sc12]

To a solution of tert-butyl (4-iodo-7-methylbenzo[d]thiazol-2-yl)carbamate (180 mg, 0.46 mmol) in dioxane (23 mL) was subsequentially added KOAc (140 mg, 1.43 mmol), B₂Pin₂ (965 mg, 3.80 mmol) and Pd(dppf)₂Cl₂ (64 mg, 0.087 mmol) at room temperature. The solution was stirred at 100° C. for overnight. Water was added to the reaction and the solution was extracted by ethyl acetate, washed by brine, dried over anhydrous Na₂SO₄, filtered and concentrated in vacuo. The residual was purified by chromatography (100% PE to 100% EA) to give 232 mg crude product which was used directly in the next step. MS (ESI, m/e) [M+H]⁺ 309.3.

### Step 2: tert-butyl 3-(7-(2-((tert-butoxycarbonyl)amino)-7 methyl benzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate

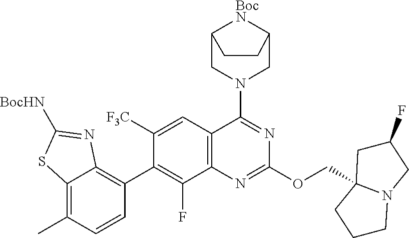 [SMILES: Cc1ccc(-c2c(C(F)(F)F)cc3c(N4CC5CCC(C4)N5C(=O)OC(C)(C)C)nc(OC[C@@]45CCCN4C[C@H](F)C5)nc3c2F)c2nc(NC(=O)OC(C)(C)C)sc12]

To a solution of tert-butyl 3-(7-bromo-8-fluoro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate (49 mg, 0.074 mmol) in dioxane/H₂O (20 mL+6 mL) was subsequentially added crude (2-((tert-butoxycarbonyl)amino)-7-methylbenzo[d]thiazol-4-yl)boronic acid (232 mg), Pd(dtbpf)₂Cl₂ (15 mg, 0.023 mmol), K₃PO₄ (127 mg, 0.60 mmol) at room temperature. The mixture was stirred at 90° C. for 2 hrs. After cooled to room temperature, Pd(dtbpf)₂Cl₂ (10 mg, 0.015 mmol) was added to the solution and the solution was stirred at 90° C. for overnight. Water was added to the reaction and the solution was extracted by ethyl acetate, washed by brine, dried over anhydrous Na₂SO₄, filtered and concentrated in vacuo. The residual was purified by chromatography (general eluent gradient PE/EA=2/1 to DCM/MeOH=4/1, then 100% MeOH) to give crude product (144 mg). MS (ESI, m/e) [M+H]⁺ 846.7.

### Step 3: 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-methylbenzoI[d]thiazol-2-amine

To a solution of crude tert-butyl 3-(7-(2-((tert-butoxycarbonyl)amino)-7-methylbenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate (144 mg) in DCM (20 mL) was added TFA (4 mL) at room temperature. The mixture was stirred at room temperature for about 4.5 h. Then, the mixture was evaporated in vacuo. The residue was redissolved in MeOH, and 7 M NH₃ in MeOH was added to adjust pH >7. Next, the solvent was removed in vacuo and the residue was purified by Pre-HPLC to give the product (1.18 mg, FA salt). ¹H NMR (500 MHz, CD₃OD) δ 8.47 (s, 2H), 8.14 (s, 1H), 7.10 (d, J=7.4 Hz, 1H), 7.02 (d, J=7.7 Hz, 1H), 5.42 (d, J=52.6 Hz, 1H), 4.66 (d, J=7.0 Hz, 1H), 4.59 (d, J=12.8 Hz, 1H), 4.50 (d, J=11.6 Hz, 1H), 4.43 (d, J=11.5 Hz, 1H), 3.98-3.88 (m, 2H), 3.85-3.73 (m, 2H), 3.64 (d, J=11.7 Hz, 1H), 3.59-3.50 (m, 2H), 3.27-3.20 (m, 1H), 2.50 (s, 3H), 2.46-2.38 (m, 2H), 2.30-2.24 (m, 1H), 2.22-2.13 (m, 2H), 2.10-1.85 (m, 5H). MS (ESI, m/e) [M+H]⁺ 646.4.

### Example 74: 4-(4-(3,6-diazabicyclo[3.1.1]heptan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

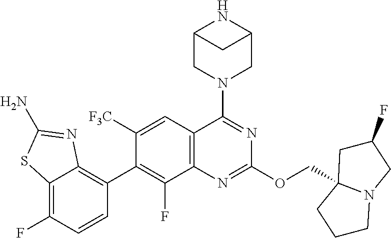 [SMILES: Nc1nc2c(-c3c(C(F)(F)F)cc4c(N5CC6CC(C5)N6)nc(OC[C@@]56CCCN5C[C@H](F)C6)nc4c3F)ccc(F)c2s1]

Synthetic Route

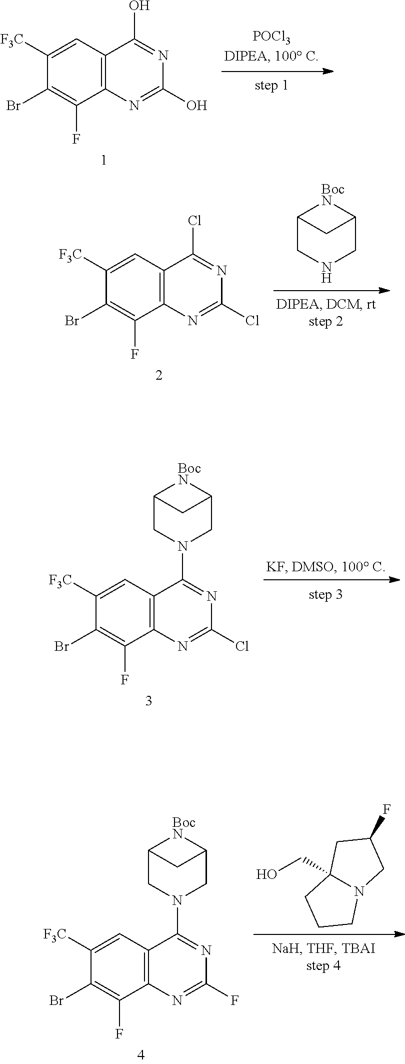 [SMILES: Oc1nc(O)c2cc(C(F)(F)F)c(Br)c(F)c2n1 ; Fc1c(Br)c(C(F)(F)F)cc2c(Cl)nc(Cl)nc12 ; CC(C)(C)OC(=O)N1C2CNCC1C2 ; CC(C)(C)OC(=O)N1C2CC1CN(c1nc(Cl)nc3c(F)c(Br)c(C(F)(F)F)cc13)C2 ; CC(C)(C)OC(=O)N1C2CC1CN(c1nc(F)nc3c(F)c(Br)c(C(F)(F)F)cc13)C2 ; OC[C@@]12CCCN1C[C@H](F)C2]

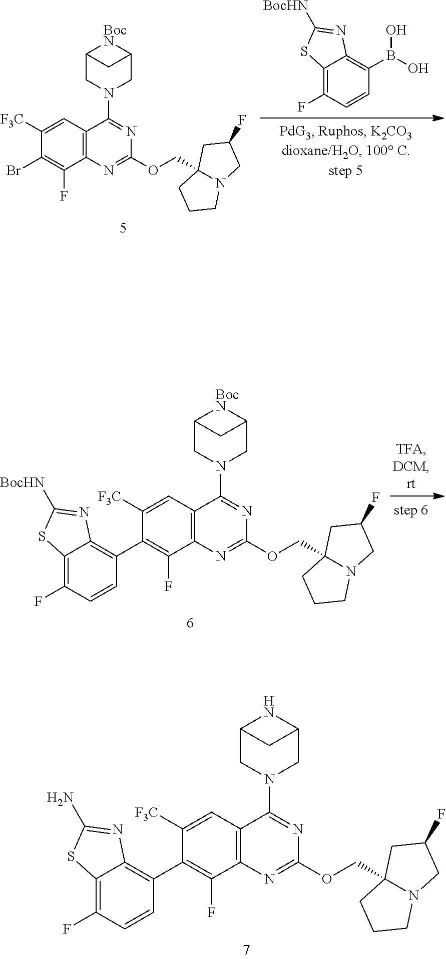 [SMILES: CC(C)(C)OC(=O)N1C2CC1CN(c1nc(OC[C@@]34CCCN3C[C@H](F)C4)nc3c(F)c(Br)c(C(F)(F)F)cc13)C2 ; CC(C)(C)OC(=O)Nc1nc2c(B(O)O)ccc(F)c2s1 ; CC(C)(C)OC(=O)Nc1nc2c(-c3c(C(F)(F)F)cc4c(N5CC6CC(C5)N6C(=O)OC(C)(C)C)nc(OC[C@@]56CCCN5C[C@H](F)C6)nc4c3F)ccc(F)c2s1 ; Nc1nc2c(-c3c(C(F)(F)F)cc4c(N5CC6CC(C5)N6)nc(OC[C@@]56CCCN5C[C@H](F)C6)nc4c3F)ccc(F)c2s1]

### Step 1: tert-butyl 3-(7-bromo-2-chloro-8-fluoro-6-(trifluoromethyl)quinazolin-4-yl)-3,6-diazabicyclo[3.1.1]heptane-6-carboxylate

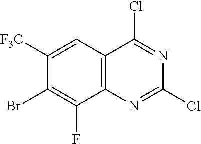 [SMILES: Fc1c(Br)c(C(F)(F)F)cc2c(Cl)nc(Cl)nc12]

To a solution of 7-bromo-8-fluoro-6-(trifluoromethyl)quinazoline-2,4-diol (363 mg, 1.11 mmol) in POCl₃ (10 mL) was added DIPEA (2 mL) at room temperature. The mixture was stirred at 100° C. for about 2.5 hrs. The mixture was evaporated in vacuo to remove POCl₃ and ethyl acetate was added to dilute the reaction. The mixture was poured into ice water, extracted by ethyl acetate, washed by brine, dried over anhydrous Na₂SO₄, filtered and evaporated in vacuo to afford the crude product used directly in the next step. MS (ESI, m/e) [M+H]⁺ 363.0.

### Step 2: tert-butyl 3-(7-bromo-2-chloro-8-fluoro-6-(trifluoromethyl)quinazolin-4-yl)-3,6-diazabicyclo[3.1.1]heptane-6-carboxylate

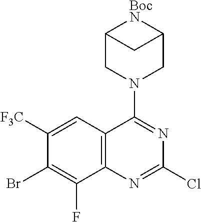 [SMILES: CC(C)(C)OC(=O)N1C2CC1CN(c1nc(Cl)nc3c(F)c(Br)c(C(F)(F)F)cc13)C2]

To a solution of crude tert-butyl 3-(7-bromo-2-chloro-8-fluoro-6-(trifluoromethyl) quinazolin-4-yl)-3,6-diazabicyclo[3.1.1]heptane-6-carboxylate in DCM (22 mL) was added tert-butyl 3,6-diazabicyclo[3.1.1]heptane-6-carboxylate (220 mg, 1.11 mmol) and DIPEA (1.2 mL, 6.66 mmol) at room temperature, respectively. The mixture was stirred at room temperature for about 3.5 hrs. Silical gel was added to the reaction and the solution was evaporated in vacuo. The solid silical gel mixture was purified by chromatography to give the product tert-butyl 3-(7-bromo-2-chloro-8-fluoro-6-(trifluoromethyl)quinazolin-4-yl)-3,6-diazabicyclo[3.1.1]heptane-6-carboxylate (414 mg) used directly in the next step. MS (ESI, m/e) [M+H]⁺ 525.3.

### Step 3: tert-butyl 3-(7-bromo-2,8-difluoro-6-(trifluoromethyl)quinazolin-4-yl)-3,6-diazabicyclo[3.1.1]heptane-6-carboxylate

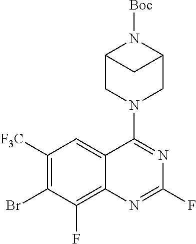 [SMILES: CC(C)(C)OC(=O)N1C2CC1CN(c1nc(F)nc3c(F)c(Br)c(C(F)(F)F)cc13)C2]

To a solution of tert-butyl 3-(7-bromo-2-chloro-8-fluoro-6-(trifluoromethyl)quinazolin-4-yl)-3,6-diazabicyclo[3.1.1]heptane-6-carboxylate (413 mg, 0.79 mmol) in DMSO (25 mL) was added KF (455 mg, 7.85 mmol) at room temperature. The mixture was stirred at 100° C. for overnight. After cooled to room temperature, the mixture was filtered and evaporated in vacuo to give the crude product tert-butyl 3-(7-bromo-2,8-difluoro-6-(trifluoromethyl)quinazolin-4-yl)-3,6-diazabicyclo[3.1.1] heptane-6-carboxylate used directly in the next step. MS (ESI, m/e) [M+H]⁺ 509.2.

### Step 4: tert-butyl 3-(7-bromo-8-fluoro-2-(((2R,7aS)-2-fluorotetrahydro-1H-pyrrolizin-7a(5H)-yl)methoxy)-6-(trifluoromethyl)quinazolin-4-yl)-3,6-diazabicyclo[3.1.1]heptane-6-carboxylate

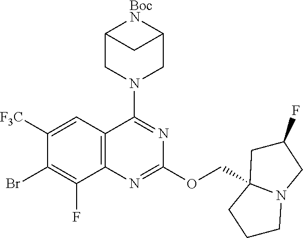 [SMILES: CC(C)(C)OC(=O)N1C2CC1CN(c1nc(OC[C@@]34CCCN3C[C@H](F)C4)nc3c(F)c(Br)c(C(F)(F)F)cc13)C2]

To a solution of 1-(piperidin-1-yl)propan-2-ol (127 mg, 0.80 mmol) in THF (20 mL) was added TBAI (29 mg, 0.079 mmol) and NaH (94 mg, 2.36 mmol) at room temperature. The mixture was stirred at room temperature for about 50 mins. Then, crude tert-butyl 3-(7-bromo-2,8-difluoro-6-(trifluoromethyl)quinazolin-4-yl)-3,6-diazabicyclo[3.1.1] heptane-6-carboxylate in THF (20 mL) was added to the mixture. The mixture was stirred at 40° C. for 1 h and quenched by saturated NH₄Cl aqueous solution, extracted by ethyl acetate. The organic layers were washed by brine, dried over anhydrous Na₂SO₄, evaporated in vacuo. The residue was purified by chromatography (PE/EA=2:1 to DCM/MeOH=4/1) to give the product (342 mg). MS (ESI, m/e) [M+H]⁺ 648.4.

### Step 5: tert-butyl 3-(7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl) quinazolin-4-yl-3,6-diazabicyclo[3.1.1]heptane-6-carboxylate

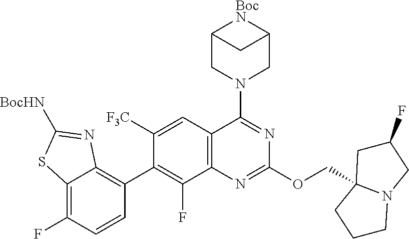 [SMILES: CC(C)(C)OC(=O)Nc1nc2c(-c3c(C(F)(F)F)cc4c(N5CC6CC(C5)N6C(=O)OC(C)(C)C)nc(OC[C@@]56CCCN5C[C@H](F)C6)nc4c3F)ccc(F)c2s1]

To a solution of tert-butyl 3-(7-bromo-8-fluoro-2-(((2R,7aS)-2-fluorotetrahydro-1H-pyrrolizin-7a(5H)-yl)methoxy)-6-(trifluoromethyl)quinazolin-4-yl)-3,6-diazabicyclo[3.1.1] heptane-6-carboxylate (20 mg, 0.031 mmol) in dioxane/H₂O (10 mL/3 mL) was added tert-butyl (7-fluoro-4-(4,4,5,5-tetramethyl-1,3,2-dioxaborolan-2-yl) benzo[d] thiazol-2-yl) carbamate (19 mg, 0.062 mmol), Ruphos-PdG₃ (3 mg, 0.0031 mmol), Ruphos (5 mg, 0.011 mmol), K₂CO₃ (16 mg, 0.12 mmol) at room temperature. The mixture was stirred at 100° C. for overnight. The mixture was evaporated in vacuo and purified by chromatography (general gradient: DCM to 5% MeOH in DCM, 10% MeOH in DCM, 20% MeOH in DCM, then 100% MeOH) to give the title product (33 mg). MS (ESI, m/e) [M+H]⁺ 836.6.

### Step 5: 4-(4-(3,6-diazabicyclo[3.1.1]heptan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-(trifluoro methyl) quinazolin-4-yl)-3,6-diazabicyclo[3.1.1]heptane-6-carboxylate (90 mg, 0.11 mmol) in DCM (20 mL) was added TFA (4 mL) at room temperature. The mixture was stirred at room temperature for about 24 hrs. Then, the mixture was evaporated in vacuo. The residue was redissolved in MeOH, and 7 M NH₃ in MeOH was added to adjust pH >7. Then, the solvent was removed in vacuo and the residue was purified by Pre-HPLC to give the product (14.18 mg, FA salt). ¹H NMR (500 MHz, CD₃OD) δ 8.61 (s, 1H), 8.43 (s, 1H), 7.27-7.15 (m, 1H), 6.98 (t, J=8.8 Hz, 1H), 5.47 (d, J=52.5 Hz, 1H), 4.70 (d, J=13.1 Hz, 2H), 4.60 (dd, J=11.9, 1.7 Hz, 1H), 4.52 (dd, J=13.7, 7.9 Hz, 3H), 4.37 (d, J=6.1 Hz, 2H), 3.85-3.58 (m, 3H), 3.50-3.33 (m, 1H), 3.07-2.95 (m, 1H), 2.62-2.41 (m, 2H), 2.38-2.28 (m, 1H), 2.28-2.18 (m, 2H), 2.14-2.01 (m, 1H), 1.91 (d, J=10.2 Hz, 1H). MS (ESI, m/e) [M+H]⁺ 636.4.

### Example 75: 4-(4-(3,8-diazabicyclo[3.2.1]octan-3-yl)-6-chloro-8-fluoro-2-(((2R,7aS)-2-fluorotetrahydro-H-pyrrolizin-7a(5H)-yl)methoxy)quinazolin-7-yl)-5,7-difluorobenzo[d]thiazol-2-amine

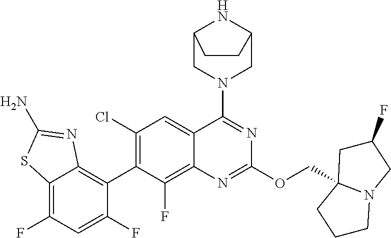 [SMILES: Nc1nc2c(-c3c(Cl)cc4c(N5CC6CCC(C5)N6)nc(OC[C@@]56CCCN5C[C@H](F)C6)nc4c3F)c(F)cc(F)c2s1]

Synthetic Route

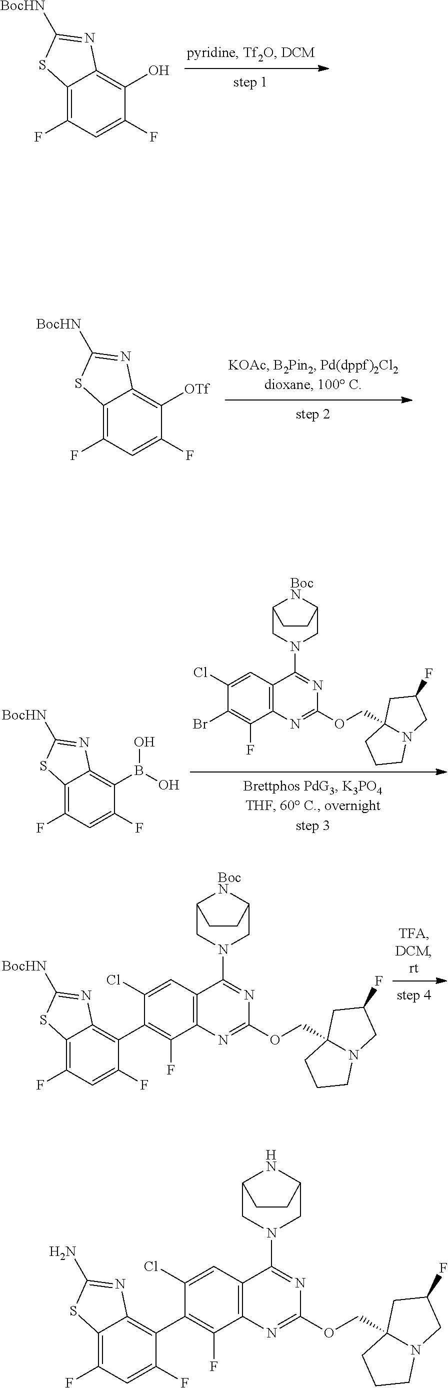 [SMILES: CC(C)(C)OC(=O)Nc1nc2c(O)c(F)cc(F)c2s1 ; CC(C)(C)OC(=O)Nc1nc2c(OS(=O)(=O)C(F)(F)F)c(F)cc(F)c2s1 ; CC(C)(C)OC(=O)Nc1nc2c(B(O)O)c(F)cc(F)c2s1 ; CC(C)(C)OC(=O)N1C2CCC1CN(c1nc(OC[C@@]34CCCN3C[C@H](F)C4)nc3c(F)c(Br)c(Cl)cc13)C2 ; CC(C)(C)OC(=O)Nc1nc2c(-c3c(Cl)cc4c(N5CC6CCC(C5)N6C(=O)OC(C)(C)C)nc(OC[C@@]56CCCN5C[C@H](F)C6)nc4c3F)c(F)cc(F)c2s1 ; Nc1nc2c(-c3c(Cl)cc4c(N5CC6CCC(C5)N6)nc(OC[C@@]56CCCN5C[C@H](F)C6)nc4c3F)c(F)cc(F)c2s1]

### Step 1: 2-((tert-butoxycarbonyl)amino)-5,7-difluorobenzo[d]thiazol-4-yl trifluoro methanesulfonate

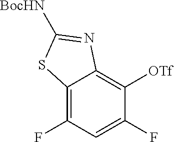 [SMILES: CC(C)(C)OC(=O)Nc1nc2c(OS(=O)(=O)C(F)(F)F)c(F)cc(F)c2s1]

To a solution of tert-butyl (5,7-difluoro-4-hydroxybenzo[d]thiazol-2-yl)carbamate (6.04 g, 20 mmol) and pyridine (3.2 mL, 40 mmol) in DCM (100 mL) was added dropwise Tf₂O (6.77 g, 24 mmol) at 0° C. The resulting mixture was stirred at room temperature for 2 hrs. After completion, the reaction mixture was diluted with DCM, washed with water. The organic layer was dried over anhydrous Na₂SO₄, filtered and the filtrate was concentrated to give a residue. the residue was purified by silica gel column chromatography, eluted with (PE:EA=4:1) to give the title product (7.70 g). MS (ESI, m/e) [M−56]⁺379.

### Step 2: (2-((tert-butoxycarbonyl)amino)-5,7-difluorobenzo[d]thiazol-4-yl)boronic acid

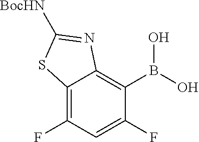 [SMILES: CC(C)(C)OC(=O)Nc1nc2c(B(O)O)c(F)cc(F)c2s1]

To a solution of 2-((tert-butoxycarbonyl)amino)-5,7-difluorobenzo[d]thiazol-4-yl trifluoromethanesulfonate (2 g, 4.63 mmol) in dioxane (46 mL) was subsequentially added KOAc (1.32 g, 13.43 mmol), B₂Pin₂ (9.50 g, 37.5 mmol) and Pd(dppf)₂Cl₂.DCM (577 mg, 0.71 mmol) at room temperature. The solution was stirred at 100° C. for overnight. After cooled to ambident temperature, B₂Pin₂ (2.94 g, 11.6 mmol) and Pd(dppf)₂Cl₂.DCM (561 mg, 0.69 mmol) was subsequentially added to the solution. The solution was stirred at 100° C. for 24 h. Then, water was added to the reaction. The solution was extracted by ethyl acetate, washed by brine, dried over anhydrous Na₂SO₄, filtered and concentrated in vacuo. The residual was purified by chromatography (the general eluent gradient: 100% PE to 100% EA) to give the product (2-((tert-butoxycarbonyl)amino)-5,7-difluorobenzo[d]thiazol-4-yl) boronic acid (418 mg). MS (ESI, m/e) [M+H]⁺ 331.3.

### Step 3: tert-butyl 3-(7-(2-((tert-butoxycarbonyl)amino)-5,7-difluorobenzo[d] thiazol-4-yl)-6-chloro-8-fluoro-2-(((2R,7aS)-2-fluorotetrahydro-1H-pyrrolizin-7a(5H)-yl) methoxy)quinazolin-4-yl)-3,8-diazabicyclo[3.2.1]octane-8-carboxylate

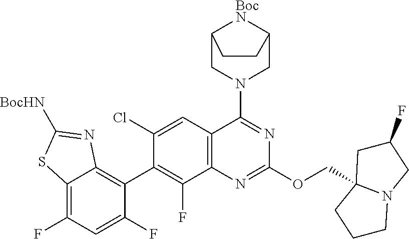 [SMILES: CC(C)(C)OC(=O)Nc1nc2c(-c3c(Cl)cc4c(N5CC6CCC(C5)N6C(=O)OC(C)(C)C)nc(OC[C@@]56CCCN5C[C@H](F)C6)nc4c3F)c(F)cc(F)c2s1]

To a solution of tert-butyl 3-(7-bromo-6-chloro-8-fluoro-2-(((2R,7aS)-2-fluoro tetrahydro-1H-pyrrolizin-7a(5H)-yl)methoxy)quinazolin-4-yl)-3,8-diazabicyclo[3.2.1] octane-8-carboxylate (50 mg, 0.08 mmol) in THF (8 mL) was subsequentially added (2-((tert-butoxycarbonyl)amino)-5,7-difluorobenzo[d]thiazol-4-yl)boronic acid (37 mg, 0.112 mmol), Brettphos-PdG₃ (7 mg, 0.0077 mmol), K₃PO₄ (51 mg, 0.24 mmol) at room temperature. The mixture was stirred at 60° C. for about 21 hrs. After cooled to room temperature, Brettphos-PdG3 (11 mg, 0.012 mmol), (2-((tert-butoxy carbonyl)amino)-5,7-difluorobenzo[d]thiazol-4-yl)boronic acid (13 mg, 0.039 mmol) and THF (8 mL) was subsequentially added to the solution. The solution was stirred at 60° C. for 20.5 h. The mixture was filtered and the filtrate was evaporated in vacuo. The residual was purified by chromatography (general eluent gradient: PE/EA=2/1 to DCM/MeOH=4/1, then 100% MeOH) to give crude product (25 mg). MS (ESI, m/e) [M+H]⁺ 834.4.

### Step 4: 4-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-5,7 difluorobenzo[d] thiazol-2-amine

To a solution of crude tert-butyl 3-(7-(2-((tert-butoxycarbonyl)amino)-5,7-difluorobenzo[d] thiazol-4-yl)-6-chloro-8-fluoro-2-(((2R,7aS)-2-fluorotetrahydro-1H-pyrrolizin-7a(5H)-yl) methoxy)quinazolin-4-yl)-3,8-diazabicyclo[3.2.1]octane-8-carboxylate (25 mg) in DCM (20 mL) was added TFA (4 mL) at room temperature. The mixture was stirred at room temperature for about 24 hrs. Then, the mixture was evaporated in vacuo. The residue was redissolved in MeOH, and 7 M NH₃ in MeOH was added to adjust pH >7. Then, the solvent was removed in vacuo and the residue was purified by Pre-HPLC to give the product (0.74 mg, FA salt). ¹H NMR (500 MHz, CD₃OD) δ 8.48 (s, 2H), 7.93 (s, 1H), 6.92 (t, J=9.7 Hz, 1H), 5.42 (d, J=52.9 Hz, 1H), 4.64-4.55 (m, 2H), 4.43 (dd, J=31.9, 11.4 Hz, 2H), 3.95-3.85 (m, 2H), 3.74 (dd, J=17.8, 5.9 Hz, 2H), 3.62 (d, J=13.8 Hz, 1H), 3.57-3.42 (m, 2H), 3.25-3.20 (m, 1H), 2.53-2.33 (m, 2H), 2.30-2.23 (m, 1H), 2.19-2.11 (m, 2H), 2.06-1.91 (m, 5H). MS (ESI, m/e) [M+H]⁺ 634.3.

### Example 76: 4-(4-(3,8-diazabicyclo[3.2.1]octan-3-yl)-2-(((2R,7aS)-2-fluorotetrahydro-1H-pyrrolizin-7a(5H)-yl)methoxy)-8-methyl-6-(trifluoromethyl)quinazolin-7-yl)-7-fluorobenzo[d]thiazol-2-amine

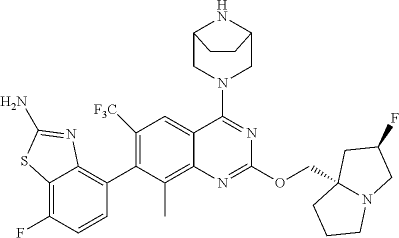 [SMILES: Cc1c(-c2ccc(F)c3sc(N)nc23)c(C(F)(F)F)cc2c(N3CC4CCC(C3)N4)nc(OC[C@@]34CCCN3C[C@H](F)C4)nc12]

Synthetic Route

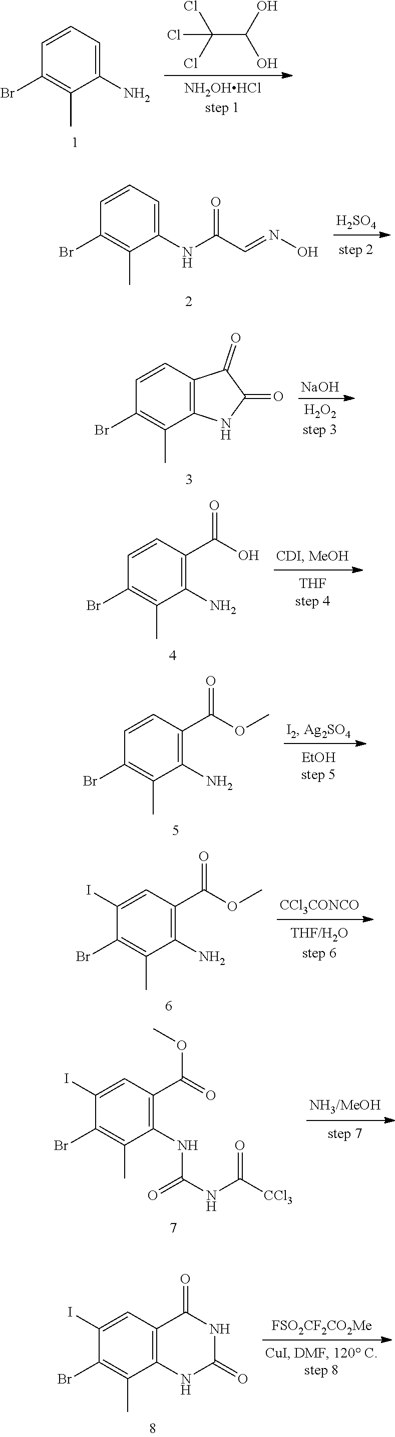

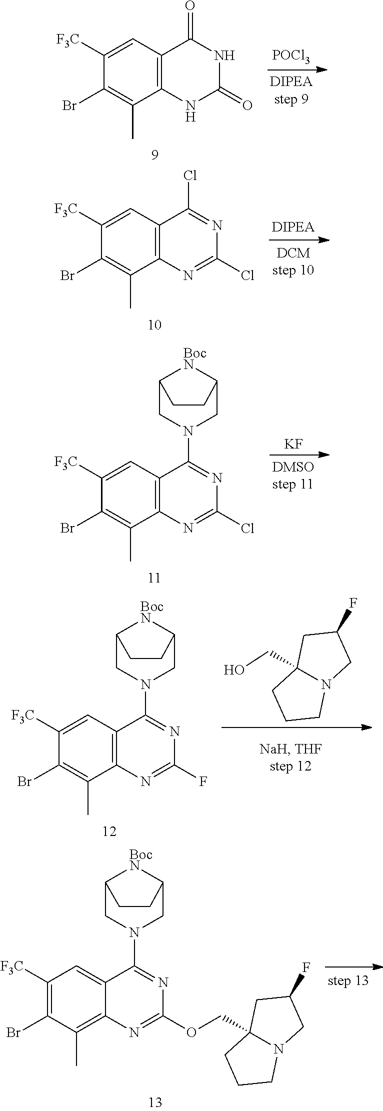

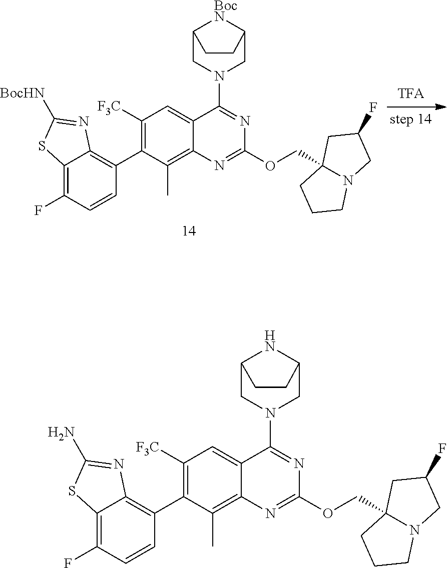 [SMILES: Cc1c(-c2ccc(F)c3sc(NC(=O)OC(C)(C)C)nc23)c(C(F)(F)F)cc2c(N3CC4CCC(C3)N4C(=O)OC(C)(C)C)nc(OC[C@@]34CCCN3C[C@H](F)C4)nc12 ; Cc1c(-c2ccc(F)c3sc(N)nc23)c(C(F)(F)F)cc2c(N3CC4CCC(C3)N4)nc(OC[C@@]34CCCN3C[C@H](F)C4)nc12]

### Step 1: (N-(3-bromo-2-methylphenyl)-2-(hydroxyimino)acetamide

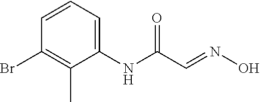

Chloral hydrate (5.35 g, 32.8 mmol) and Na₂SO₄ (1 g) were dissolved in H₂O (70 mL) in a 500 mL glass-lined reactor and heated to 35° C., then 3-bromo-2-methylaniline (5 g, 27.3 mmol) in H₂O (30 mL) and concentrated HCl (3 mL) were warmed and added to the reaction mixture. The resulting mixture was stirred at 35° C. for 2 hours. NH₂OH—HCl (7.83 g, 111.9 mmol) was added to the reaction mixture in portions. The result mixture was heated to 50° C. and stirred for 16 hours, The reaction mixture was cooled to room temperature and filtered. The solid was washed with H₂O (2 L) and dried to give the title compound (4.1 g). MS (ESI, m/e) [M+1]⁺257.0

### Step 2: 6-bromo-7-methylindoline-2,3-dione

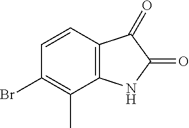

To a solution of (N-(3-bromo-2-methylphenyl)-2-(hydroxyimino)acetamide (4 g, 15.56 mmol) in concentrated H₂SO₄ (30 mL) was stirred at 50° C. for 2 hrs. The reaction mixture was cooled to room temperature and poured into ice water. The solid was collected by filtration and washed with H₂O and dried to give the title compound (3.0 g). MS (ESI, m/e) [M+1]⁺240.0.

### Step 3: 2-amino-4-bromo-3-methylbenzoic acid

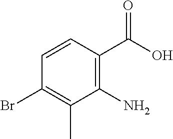

NaOH (680 mg, 21.2 mmol) was added into H₂O (10 mL) was added to 6-bromo-7-methylindoline-2,3-dione (2 g, 9 mmol) at room temperature. The result mixture was cooled to 0-5° C., H₂O₂ (4.4 mL) was added drop-wise to the mixture at 0-5° C. After the complete of the addition, the reaction was warmed to room temperature and stirred overnight. The reaction mixture was extracted with MTBE (50 mL×2), The aqueous layer was acidified to pH=6.0 with con.HCl, and the solid was collected by filtration and dried to obtain the title compound (1.3 g). MS (ESI, m/e) [M+1]⁺230.0

### Step 4: methyl 2-amino-4-bromo-3-methylbenzoate

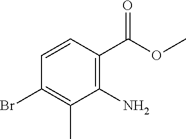

A solution of 2-amino-4-bromo-3-methylbenzoic acid (1.3 g, 5.65 mmol) in THF (50 mL) was stirred at 50° C. overnight. To the reaction mixture was added MeOH (30 mL) and stirred at room temperature for 5 hrs. The solution was evaporated and purified by chromatography column on silica gel (eluting with PE/EA=5/1) to give the title compound (1.2 g) MS (ESI, m/e) [M+1]⁺ 244.0

### Step 5: methyl 2-amino-4-bromo-5-iodo-3-methylbenzoate

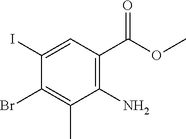

To a solution of methyl 2-amino-4-bromo-methylbenzoate (1.2 g, 4.9 mmol) and Ag₂SO₄ (1.5 g, 4.9 mmol) in EtOH (50 mL) was added I₂ (1.2 g, 4.9 mmol) at room temperature and the mixture was stirred for 5 hrs. The solution was evaporated in vacuo and purified with chromatography column on silica (eluting with PE/EA=5/1) to give the title compound (1.56 g). MS (ESI, m/e) [M+1]⁺370.0.

### Step 6: methyl 4-bromo-5-iodo-3-methyl-2-(3-(2,2,2-trichloroacetyl)ureido)benzoate

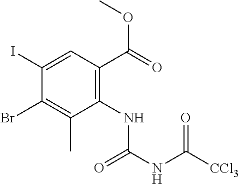

To a solution of methyl 2-amino-4-bromo-5-iodo-3-methylbenzoate (1.5 g, 4.1 mmol) in THF (50 mL) was added 2,2,2-trichloroacetyl isocyanate (1.1 g, 6.1 mmol) at room temperature and stirred for 1 h. The resulting solution was concentrated and the solid was washed with MTBE (50 mL×2) to obtain the title compound (1.85 g). MS (ESI, m/e) [M+1]⁺370.0.

### Step 7: 7-bromo-6-iodo-8-methylquinazoline-2,4(1H,3H)-dione

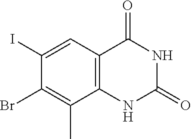

To a solution of methyl 4-bromo-5-iodo-3-methyl-2-(3-(2,2,2-trichloroacetyl)ureido)benzoate (1.6 g, 2.87 mmol) in MeOH (20 mL) was added NH₃/MeOH (3 mL) at room temperature. The reaction mixture was stirred at room temperature for 2 h. The resulting solid was filtered and washed with MTBE (50 mL×2) to obtain the title compound (1.0 g). MS (ESI, m/e) [M+1]⁺381.0.

### Step 8: 7-bromo-8-methyl-6-(trifluoromethyl)quinazoline-2,4(1H,3H)-dione

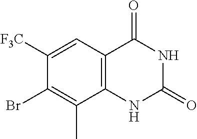 [SMILES: Cc1c(Br)c(C(F)(F)F)cc2c(=O)[nH]c(=O)[nH]c12]

To a solution of 7-bromo-6-iodo-8-methylquinazoline-2,4(1H,3H)-dione (1 g, 2.63 mmol), FSO₂CF₂CO₂Me (1.25 g, 6.58 mmol) and CuI (0.5 g, 2.63 mmol) in DMF (50 mL) was added and the mixture was stirred at 100° C. overnight. The resulting solution was concentrated and purified by chromatography column on silica (eluting with DCM/MeOH=20/1) to give the title compound (0.3 g). MS (ESI, m/e) [M+1]⁺323.0.

### Step 9: 7-bromo-2,4-dichloro-8-methyl-6-(trifluoromethyl)quinazoline

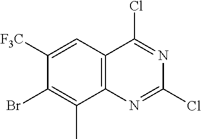 [SMILES: Cc1c(Br)c(C(F)(F)F)cc2c(Cl)nc(Cl)nc12]

To a solution of 7-bromo-8-methyl-6-(trifluoromethyl)quinazoline-2,4(1H,3H)-dione (0.3 g, 0.93 mmol) in POCl₃ (10 mL) was added one drop of DIPEA at room temperature. The reaction mixture was stirred at 100° C. for 5 h. The resulting solution was concentrated and added DCM (30 mL) and H₂O (30 mL), the aqueous layer was extracted with DCM (20 mL) and dried over Na₂SO₄, The solvent was evaporated to obtain the title compound (308 mg). MS (ESI, m/e) [M+1]⁺ no

### Step 10: tert-butyl 3-(7-bromo-2-chloro-8-methyl-6-(trifluoromethyl)quinazolin-4-yl)-3,8-diazabicyclo[3.2.1]octane-8-carboxylate

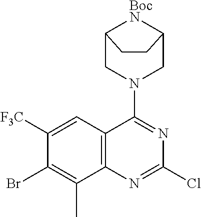 [SMILES: Cc1c(Br)c(C(F)(F)F)cc2c(N3CC4CCC(C3)N4C(=O)OC(C)(C)C)nc(Cl)nc12]

To a solution of 7-bromo-2,4-dichloro-8-methyl-6-(trifluoromethyl)quinazoline (0.2 g, 0.55 mmol) and DIPEA (0.5 mL) was added tert-butyl 3,8-diazabicyclo[3.2.1]octane-8-carboxylate (129 mg, 0.61 mmol) at room temperature. The resulting solution was stirred at room temperature for 3 hrs. The solvent was concentrated and purified by chromatography column on silica (eluting with DCM/MeOH=40/1) to give the title compound (110 mg). MS (ESI, m/e) [M+1]⁺535.0.

### Step 11: tert-butyl 3-(7-bromo-2-fluoro-8-methyl-6-(trifluoromethyl)quinazolin-4-yl)-3,8-diazabicyclo[3.2.1]octane-8-carboxylate

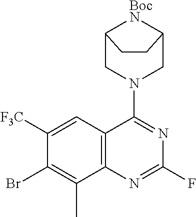 [SMILES: Cc1c(Br)c(C(F)(F)F)cc2c(N3CC4CCC(C3)N4C(=O)OC(C)(C)C)nc(F)nc12]

To a solution of tert-butyl 3-(7-bromo-2-chloro-8-methyl-6-(trifluoromethyl)quinazolin-4-yl)-3,8-diazabicyclo[3.2.1]octane-8-carboxylate (110 mg, 0.20 mmol) and KF (120 mg, 2.0 mmol) in DMSO (10 mL) at 100° C. overnight. The solvent was concentrated and purified by chromatography column on silica (eluting with DCM/MeOH=20/1) to give the title compound (35 mg). MS (ESI, m/e) [M+1]⁺519.0.

### Step 12: tert-butyl 3-(7-bromo-2-(((2R,7aS)-2-fluorotetrahydro-1H-pyrrolizin-7a(5H)-yl-methoxy)-8-methyl-6-(trifluoromethyl)quinazolin-4-yl)-3,8-diazabicyclo[3.2.1]octane-8-carboxylate

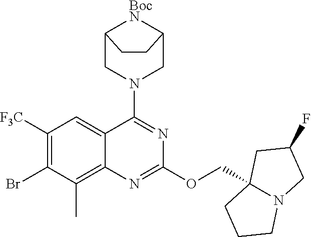 [SMILES: Cc1c(Br)c(C(F)(F)F)cc2c(N3CC4CCC(C3)N4C(=O)OC(C)(C)C)nc(OC[C@@]34CCCN3C[C@H](F)C4)nc12]

To a solution of ((2R,7aS)-2-fluorotetrahydro-1H-pyrrolizin-7a(5H)-yl)methanol (21 mg, 0.13 mmol) in THF (10 mL) was added NaH (6 mg, 0.13 mmol) at room temperature. The reaction mixture was stirred at room temperature for 1 h, tert-butyl 3-(7-bromo-2-fluoro-8-methyl-6-(trifluoromethyl)quinazolin-4-yl)-3,8-diazabicyclo[3.2.1]octane-8-carboxylate (35 mg, 0.067 mmol) was added and stirred for 1 h. The solvent was concentrated and purified by pre-TLC (eluting with DCM/MeOH=20/1) to give the title compound (25 mg). MS (ESI, m/e) [M+1]⁺658.2.

### Step 13: tert-butyl 3-(7-(2-((tert-butoxycarbonyl)amino)-7-fluorobenzo[d]thiazol-4-yl)-2-(((2R,7aS)-2-fluorotetrahydro-1H-pyrrolizin-7a(5H)-yl)methoxy)-8-methyl-6-(trifluoromethyl)quinazolin-4-yl)-3,8-diazabicyclo[3.2.1]octane-8-carboxylate

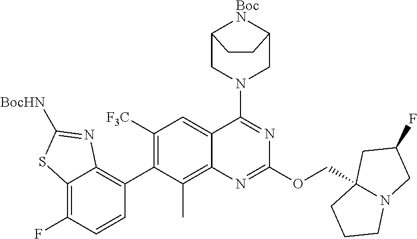 [SMILES: Cc1c(-c2ccc(F)c3sc(NC(=O)OC(C)(C)C)nc23)c(C(F)(F)F)cc2c(N3CC4CCC(C3)N4C(=O)OC(C)(C)C)nc(OC[C@@]34CCCN3C[C@H](F)C4)nc12]

A mixture of tert-butyl 3-(7-bromo-2-(((2R,7aS)-2-fluorotetrahydro-1H-pyrrolizin-7a(5H)-yl-methoxy)-8-methyl-6-(trifluoromethyl)quinazolin-4-yl)-3,8-diazabicyclo[3.2.1]octane-8-carboxylate (25 mg, 0.038 mmol), (2-((tert-butoxycarbonyl)amino)-7-fluorobenzo[d]thiazol-4-yl)boronic acid (35 mg, 0.11 mmol), Pd(dppf)₂Cl₂ (10 mg, 0.011 mmol) and NaHCO₃ (10 mg, 0.12 mmol) in dioxane/H₂O (10 mL/1 mL) was stirred at 100° C. overnight. The solvent was concentrated and purified by pre-TLC (eluting with DCM/MeOH=10/1) to give the title compound (12 mg). MS (ESI, m/e) [M+1]⁺ 846.2.

### Step 14: 4-(4-(3,8-diazabicyclo[3.2.1]octan-3-yl)-2-(((2R,7aS)-2-fluorotetrahydro-1H-pyrrolizin-7a(5H)-yl)methoxy)-8-methyl-6-(trifluoromethyl)quinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-2-(((2R,7aS)-2-fluorotetrahydro-1H-pyrrolizin-7a(5H)-yl)methoxy)-8-methyl-6-(trifluoromethyl)quinazolin-4-yl)-3,8-diazabicyclo[3.2.1]octane-8-carboxylate (12 mg, 0.014 mmol) in DCM (2 mL) was added TFA (1 mL) at room temperature. The reaction mixture was stirred at room temperature for 1 h. The solvent was concentrated and purified by pre-HPLC to give the title compound (2 mg). ¹H NMR (400 MHz, CD₃OD) δ 8.17 (s, 2H), 7.08-7.06 (m, 1H), 6.98-6.05 (m, 1H), 5.50-5.40 (m, 1H), 4.59-4.44 (m, 4H), 4.11-4.03 (m, 1H), 3.83-3.75 (m, 2H), 3.61-3.57 (m, 3H), 3.30-3.25 (m, 1H), 2.57-2.47 (m, 2H), 2.22-2.19 (m, 4H), 2.10-2.03 (m, 6H). MS (ESI, m/e) [M+1]⁺646.4.

### Example 77: 6-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-4-methyl-5-(trifluoromethyl)pyridin-2-amine

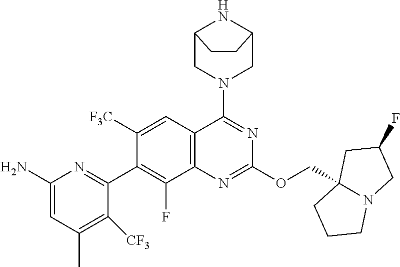 [SMILES: Cc1cc(N)nc(-c2c(C(F)(F)F)cc3c(N4CC5CCC(C4)N5)nc(OC[C@@]45CCCN4C[C@H](F)C5)nc3c2F)c1C(F)(F)F]

Synthetic Route

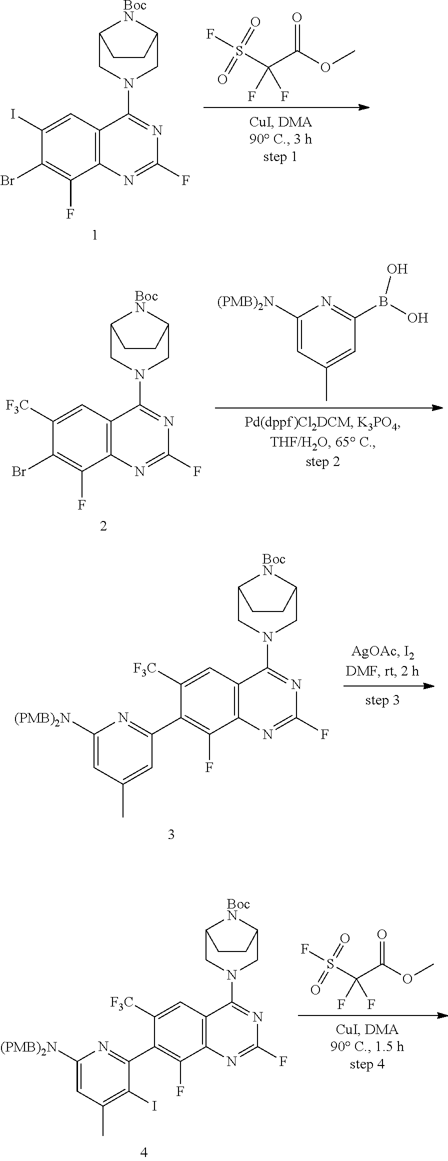

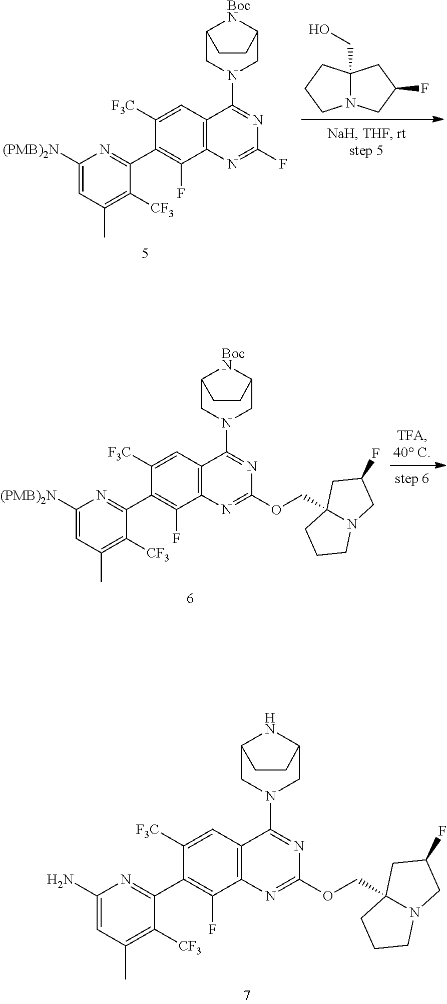

### Step 1: tert-butyl 3-(7-bromo-2,8-difluoro-6-(trifluoromethyl)quinazolin-4-yl)-3,8-diazabicyclo[3.2.1]octane-8-carboxylate

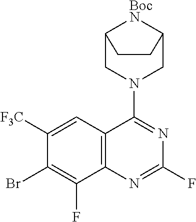 [SMILES: CC(C)(C)OC(=O)N1C2CCC1CN(c1nc(F)nc3c(F)c(Br)c(C(F)(F)F)cc13)C2]

A mixture of tert-butyl 3-(7-bromo-2,8-difluoro-6-iodoquinazolin-4-yl)-3,8-diaza bicyclo [3.2.1]octane-8-carboxylate (500 mg, 0.86 mmol), CuI (655 mg, 3.45 mmol) and methyl 2,2-difluoro-2-sulfoacetate (661 mg, 3.44 mmol) in DMA (5 mL) was stirred for 3 hrs at 90° C. The resulting mixture was extracted with EtOAc (2×200 mL). The combined organic layers were washed with brine (2×100 mL) and dried over anhydrous Na₂SO₄. After filtration, the filtrate was concentrated under reduced pressure. The residue was purified by Prep-TLC (PE/EtOAc=3:1) to afford the title product. MS (ESI, m/e) [M+H]⁺ 523.2.

### Step 2: tert-butyl 3-(7-(6-(bis(4-methoxybenzyl)amino)-4-methylpyridin-2-yl)-2,8-difluoro-6-(trifluoromethyl)quinazolin-4-yl)-3,8-diazabicyclo[3.2.1]octane-8-carboxylate

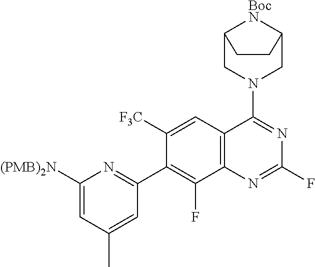

A mixture of tert-butyl 3-(7-bromo-2,8-difluoro-6-(trifluoromethyl)quinazolin-4-yl)-3,8-diazabicyclo[3.2.1]octane-8-carboxylate (100 mg, 0.19 mmol), (6-(bis(4-methoxybenzyl) amino)-4-methylpyridin-2-yl)boronic acid (149 mg, 0.38 mmol), K₃PO₄ (81 mg, 0.38 mmol) and Pd(dppf)Cl₂.DCM (15.6 mg, 0.02 mmol) in THF/H₂O (1 mL/0.2 mL) was stirred for 2 hrs at 65° C. The resulting mixture was concentrated under vacuum. The residue was purified by Prep-TLC (PE/EtOAc=3:1) to give the title product (99 mg). MS (ESI, m/e) [M+H]⁺ 791.5.

### Step 3: tert-butyl 3-(7-(6-(bis(4-methoxybenzyl)amino)-3-iodo-4-methylpyridin-2-yl)-2,8-difluoro-6-(trifluoromethyl)quinazolin-4-yl)-3,8-diazabicyclo[3.2.1]octane-8-carboxylate

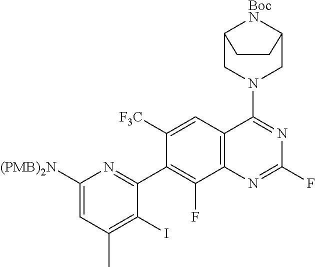

A mixture of tert-butyl 3-[7-(6-{bis[(4-methoxyphenyl)methyl]amino}-4-methylpyridin-2-yl)-2,8-difluoro-6-(trifluoromethyl)quinazolin-4-yl]-3,8-diazabicyclo[3.2.1]octane-8-carboxylate (90 mg, 0.11 mmol), AgOAc (47 mg, 0.28 mmol) and I₂ (87 mg, 0.34 mmol) in DMF (2 mL) was stirred for 2 hrs at room temperature. The resulting mixture was filtered, and the filter cake was washed with ethyl acetate. The filtrate was concentrated under reduced pressure. The residue was purified by Prep-TLC (PE/EtOAc=3:1) to give the title product (50 mg). MS (ESI, m/e) [M+H]⁺ 917.7.

### Step 4: tert-butyl 3-(7-(6-(bis(4-methoxybenzyl)amino)-4-methyl-3-(trifluoromethyl) pyridin-2-yl)-2,8-difluoro-6-(trifluoromethyl)quinazolin-4-yl)-3,8-diazabicyclo[3.2.1]octane-8-carboxylate

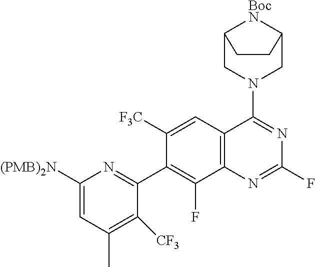

A mixture of tert-butyl 3-(7-(6-(bis(4-methoxybenzyl)amino)-3-iodo-4-methylpyridin-2-yl)-2,8-difluoro-6-(trifluoromethyl)quinazolin-4-yl)-3,8-diazabicyclo[3.2.1]octane-8-carboxylate (50 mg, 0.05 mmol), CuI (42 mg, 0.22 mmol) and methyl 2,2-difluoro-2-sulfoacetate (104.8 mg, 0.55 mmol) in DMA (1 mL) was stirred for 1.5 hrs at 90° C. The resulting mixture was filtered. The filter cake was washed with ethyl acetate. The filtrate was concentrated under reduced pressure. The residue was purified by Prep-TLC (PE/EtOAc=3:1) to give the title product (35 mg). MS (ESI, m/e) [M+H]⁺ 859.7.

### Step 5: tert-butyl 3-(7-(6-(bis(4-methoxybenzyl)amino)-4-methyl-3-(trifluoromethyl) pyridin-2-yl)-8-fluoro-2-(((2R,7aS)-2-fluorotetrahydro-1H-pyrrolizin-7a(5H)-yl)methoxy)-6-(trifluoromethyl)quinazolin-4-yl)-3,8-diazabicclo[3.2.1]octane-8-carboxylate

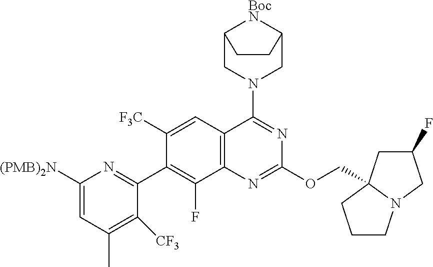

A mixture of ((2R,7aS)-2-fluoro-hexahydropyrrolizin-7a-yl)methanol (7 mg, 0.04 mmol), tert-butyl 3-(7-(6-(bis(4-methoxybenzyl)amino)-4-methyl-3-(trifluoromethyl) pyridin-2-yl)-2,8-difluoro-6-(trifluoromethyl)quinazolin-4-yl)-3,8-diazabicyclo[3.2.1]octane-8-carboxylate (35 mg, 0.04 mmol) and NaH (60 wt % in oil, 3 mg, 0.08 mmol) in THF (2 mL) was stirred for 2 hrs at rt. The resulting mixture was extracted with EtOAc (1×100 mL). The combined organic layers were washed with water (1×100 mL) and dried over anhydrous Na₂SO₄. After filtration, the filtrate was concentrated under reduced pressure. The residue was purified by Prep-TLC (DCM/MeOH=10:1) to give the title product (30 mg). MS (ESI, m/e) [M+H]⁺ 998.9.

### Step 6: 6-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-4-methyl-5-(trifluoro methyl)pyridin-2-amine

A solution of tert-butyl 3-(7-(6-(bis(4-methoxybenzyl)amino)-4-methyl-3-(trifluoro methyl) pyridin-2-yl)-8-fluoro-2-(((2R,7aS)-2-fluorotetrahydro-1H-pyrrolizin-7a(5H)-yl) methoxy)-6-(trifluoromethyl)quinazolin-4-yl)-3,8-diazabicyclo[3.2.1]octane-8-carboxylate (30 mg, 0.03 mmol) in TFA (0.5 mL) was stirred for one night at 40° C. The resulting mixture was concentrated under vacuum. The crude product (10 mg) was purified by Prep-HPLC to give the title product (5 mg). ¹H NMR (400 MHz, DMSO-d₆) δ 8.04 (s, 1H), 6.79 (s, 2H), 6.48 (s, 1H), 5.34-5.20 (m, 1H), 4.36 (d, J=12.2 Hz, 1H), 4.22 (d, J=12.0 Hz, 1H), 4.10 (dd, J=10.4, 7.5 Hz, 1H), 4.00 (dd, J=10.3, 6.4 Hz, 1H), 3.60-3.52 (m, 4H), 3.20-3.08 (m, 4H), 2.82 (d, J=7.0 Hz, 1H), 2.45 (s, 3H), 2.20-1.95 (m, 3H), 1.95-1.75 (m, 3H), 1.70-1.55 (m, 3H). MS (ESI, m/e) [M+H]⁺ 658.4.

### Example 78: 6-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-3-fluoro-4-methyl-5-(trifluoromethyl)pyridin-2-amine

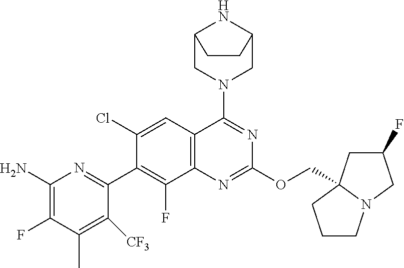 [SMILES: Cc1c(F)c(N)nc(-c2c(Cl)cc3c(N4CC5CCC(C4)N5)nc(OC[C@@]45CCCN4C[C@H](F)C5)nc3c2F)c1C(F)(F)F]

Synthetic Route

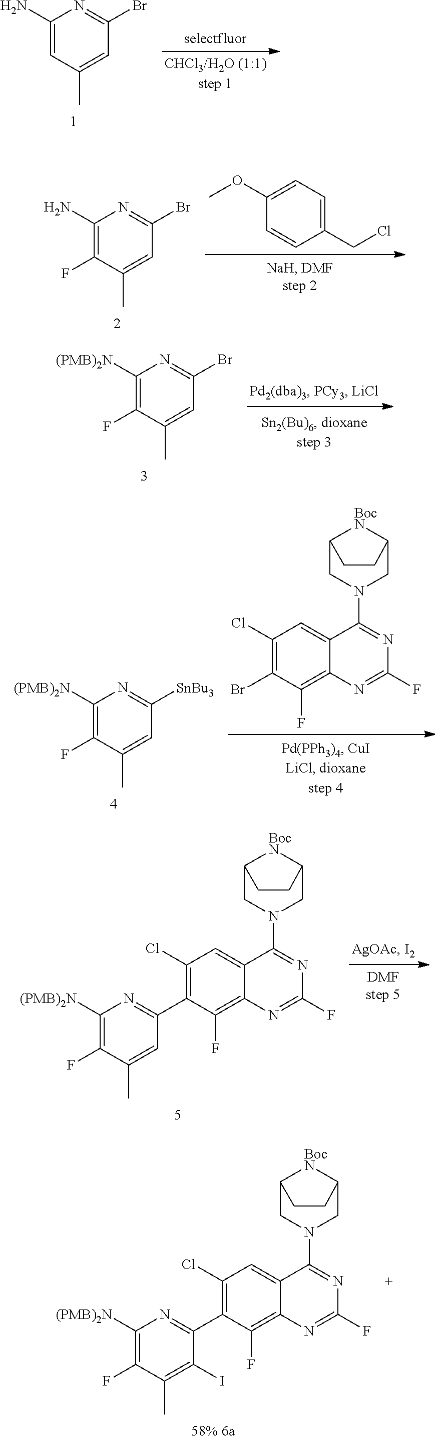

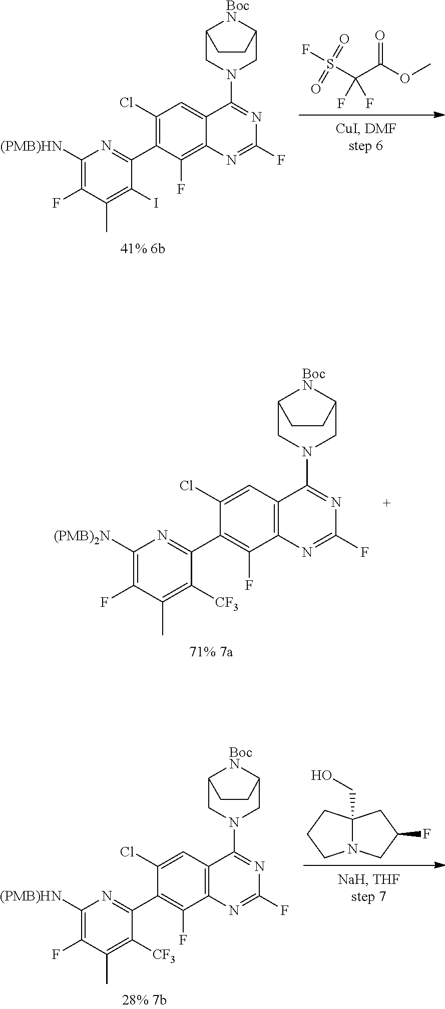

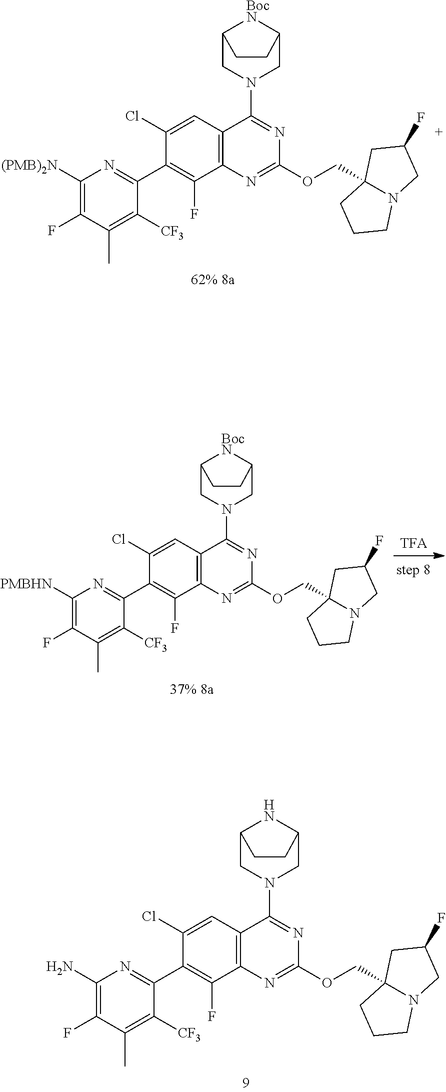

### Step 1: 6-bromo-3-fluoro-4-methylpyridin-2-amine

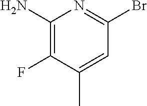

To a stirred mixture of 6-bromo-4-methylpyridin-2-amine (10 g, 53.46 mmol) in CHCl₃/H₂O (300 mL/300 mL) was added Select-Fluor (15.15 g, 42.77 mmol) in portions at 0° C., the resulting mixture was stirred overnight at room temperature. The aqueous layer was extracted by DCM (3×400 mL). The organic layer was concentrated under reduced pressure, the residue was purified by silica gel column chromatography, eluted with EtOAc/PE=(15%˜20%) to afford the title product (2.2 g). MS (ESI, m/e) [M+H]⁺ 204.9.

### Step 2: 6-bromo-3-fluoro-N,N-bis(4-methoxybenzyl)-4-methylpyridin-2-amine

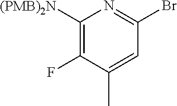

A mixture of 6-bromo-3-fluoro-4-methylpyridin-2-amine (2.2 g, 10.73 mmol) and NaH (0.64 g, 60% wt/wt, 26.83 mmol) in DMF (30 mL) was stirred for 30 min at 0° C. To the above mixture was added 4-methoxybenzyl chloride (3.70 g, 23.61 mmol) at 0° C., the resulting mixture was stirred overnight at room temperature under N₂ atmosphere. The reaction was quenched by H₂O (30 mL) and extracted with EtOAc (3×50 mL). The residue was purified by silica gel column chromatography, eluted with EtOAc/PE (8%-13%) to afford the title product (4 g). MS (ESI, m/e) [M+H]⁺ 445, 447.

### Step 3: 3-fluoro-N,N-bis(4-methoxybenzyl)-4-methyl-6-(tributylstannyl)pyridin-2-amine

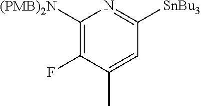

A mixture of 6-bromo-3-fluoro-N,N-bis[(4-methoxyphenyl)methyl]-4-methylpyridin-2-amine (1.5 g, 3.37 mmol), Pd₂(dba)₃ (0.31 g, 0.34 mmol), PCy₃ (0.19 g, 0.67 mmol), Sn₂(Bu)₆ (2.93 g, 5.05 mmol) and LiCl (0.71 g, 16.84 mmol) in dioxane (20 mL) was stirred overnight at 110° C. The resulting mixture was filtered, the filter cake was washed with EtOAc (3×10 mL). The filtrate was diluted by the addition of water (30 mL) and extracted with EtOAc. The organic layer was concentrated under reduced pressure and purified by silica gel column chromatography, eluted with EtOAc/PE (8%-13%) to afford the title product (2 g). MS (ESI, m/e) [M+H]⁺ 655.4.

### Step 4: tert-butyl 3-(7-(6-(bis(4-methoxybenzyl)amino)-5-fluoro-4-methylpyridin-2-yl)-6-chloro-2,8-difluoroquinazolin-4-yl)-3,8-diazabicyclo[3.2.1]octane-8-carboxylate

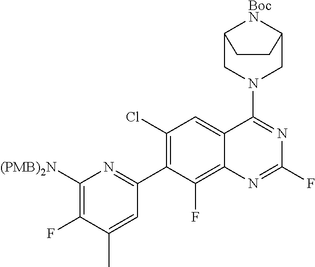

A solution of 3-fluoro-N,N-bis(4-methoxybenzyl)-4-methyl-6-(tributylstannyl)pyridin-2-amine (2 g, 3.05 mmol), tert-butyl 3-(7-bromo-6-chloro-2,8-difluoro quinazolin-4-yl)-3,8-diazabicyclo[3.2.1]octane-8-carboxylate (0.75 g, 1.53 mmol), Pd(PPh₃)₄ (0.88 g, 0.76 mmol), CuI (0.17 g, 0.870 mmol) and LiCl (0.16 g, 3.814 mmol) in dioxane (30 mL) was stirred overnight at 100° C. The resulting mixture was filtered, and the filter cake was washed with EtOAc (3×20 mL). The filtrate was concentrated under reduced pressure and purified by silica gel column chromatography, eluted with EtOAc/PE=(15%-20%) to afford the title product (500 mg). MS (ESI, m/e) [M+H]⁺ 775, 777.

### Step 5: tert-butyl 3-(7-(6-(bis(4-methoxybenzyl)amino)-5-fluoro-3-iodo-4-methyl pyridin-2-yl)-6-chloro-2,8-difluoroquinazolin-4-yl)-3,8-diazabicyclo[3.2.1]octane-8-carboxylate and tert-butyl 3-(6-chloro-2,8-difluoro-7-(5-fluoro-3-iodo-6-((4-methoxy benzyl)amino)-4-methyl pyridin-2-yl)quinazolin-4-yl)-3,8-diazabicyclo[3.2.1]octane-8-carboxylate

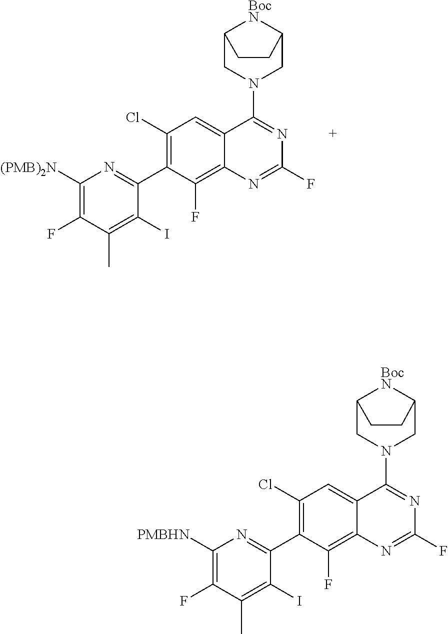

A solution of tert-butyl 3-(7-(6-(bis(4-methoxybenzyl)amino)-5-fluoro-4-methylpyridin-2-yl)-6-chloro-2,8-difluoroquinazolin-4-yl)-3,8-diazabicyclo[3.2.1]octane-8-carboxylate (0.5 g, 0.65 mmol) in DMF (5 mL) was added silver acetate (1.08 g, 6.45 mmol) and I₂ (1.31 g, 5.16 mmol) at room temperature. After stirring overnight at room temperature. The reaction was quenched with saturated Na₂S₂O₃ aqueous solution at rt. The resulting mixture was filtered, and the filter cake was washed with EtOAc (3×10 mL). The filtrate was concentrated under reduced pressure. The organic layer was concentrated under reduced pressure and purified by Prep-TLC (PE:EtOAc=3:1). This resulted in a mixture of both title products (330 mg). MS (ESI, m/e) [M+H]⁺ 901.4, MS (ESI, m/e) [M+2H]²⁺/2 392.0.

### Step 6: tert-butyl 3-(7-(6-(bis(4-methoxybenzyl)amino)-5-fluoro-4-methyl-3-(trifluoro methyl)pyridin-2-yl)-6-chloro-2,8-difluoroquinazolin-4-yl)-3,8-diazabicyclo[3.2.1] octane-8-carboxylate and tert-butyl 3-(6-chloro-2,8-difluoro-7-(5-fluoro-6-((4-methoxybenzyl) amino)-4-methyl-3-(trifluoromethyl)pyridin-2-yl)quinazolin-4-yl)-3,8-diazabicyclo[3.2.1] octane-8-carboxylate

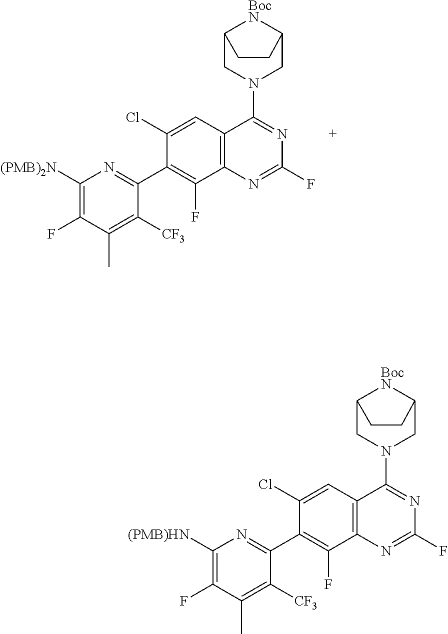

A solution of tert-butyl 3-(7-(6-(bis(4-methoxybenzyl)amino)-5-fluoro-3-iodo-4-methyl pyridin-2-yl)-6-chloro-2,8-difluoroquinazolin-4-yl)-3,8-diazabicyclo[3.2.1]octane-8-carboxylate and tert-butyl 3-(6-chloro-2,8-difluoro-7-(5-fluoro-3-iodo-6-((4-methoxy benzyl) amino)-4-methylpyridin-2-yl)quinazolin-4-yl)-3,8-diazabicyclo[3.2.1]octane-8-carboxylate (330 mg) in DMF (5 mL) was added methyl 2,2-difluoro-2-sulfoacetate (704 mg, 3.66 mmol) and CuI (277.0 mg, 1.45 mmol) at room temperature. After stirring for 3 h at 90° C., the resulting mixture was filtered, and the filter cake was washed with EtOAc (3×5 mL). The filtrate was diluted by the addition of water (25 mL) and extracted with EtOAc. The organic layer was concentrated under reduced pressure and purified by Prep-TLC (PE:EtOAc=3:1). This resulted in a mixture of both products (160 mg). MS (ESI, m/e) [M+H]⁺ 843.5, 723.4.

### Step 7: tert-butyl 3-(7-(6-(bis(4-methoxybenzyl)amino)-5-fluoro-4-methyl-3-(trifluoro methyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate and tert-butyl 3-(6-chloro-8-fluoro-7-(5-fluoro-6-((4-methoxybenzyl)amino)-4-methyl-3-(trifluoromethyl)pyridin-2-yl)-2-(((2R,7aS)-2-fluorotetrahydro-1H-pyrrolizin-7a(5H)-yl) methoxy)quinazolin-4-yl)-3,8-diazabicyclo[3.2.1]octane-8-carboxylate

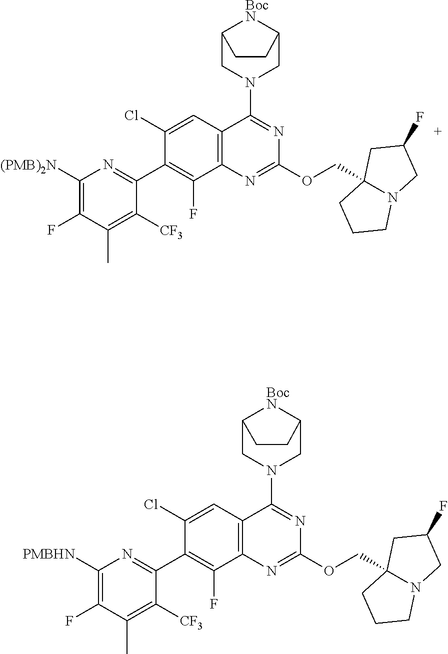

A solution of ((2R,7aS)-2-fluoro-hexahydropyrrolizin-7a-yl)methanol (57 mg, 0.36 mmol) and NaH (17 mg, 60% wt/wt, 0.72 mmol) in THF (1.5 mL) was stirred for 1 h at 0° C. To the mixture was added the above mixture (150 mg). The resulting mixture was stirred for additional 2 hrs at rt. The reaction was quenched with H₂O (5 mL) at 0° C. and extracted with EtOAc (3×5 mL). The organic layer was concentrated under reduced pressure and purified by Prep-TLC (PE:EtOAc=5:1). This resulted in a mixture of both products (100 mg). MS (ESI, m/e) [M+H]⁺ 862.6, MS (ESI, m/e) [M+2H]²⁺/2 492.0.

### Step 8: 6-(4-(3,8-diazabicyclo[3.2.1]octan-3-yl)-6-chloro-8-fluoro-2-(((2R,7aS)-2-fluoro tetrahydro-1H-pyrrolizin-7a(5H)-yl)methoxy)quinazolin-7-yl)-3-fluoro-4-methyl-5-(trifluoro methyl)pyridin-2-amine

A solution of the product above (90 mg) in TFA (2 mL) was stirred for 2 hrs at 50° C. The resulting mixture was concentrated under reduced pressure. The crude product was purified by Prep-HPLC to afford the title product (24.9 mg). MS (ESI, m/e) [M+H]⁺ 642.2. ¹H NMR (400 MHz, DMSO-d₆) δ 7.82 (s, 1H), 7.23 (s, 2H), 5.46-5.04 (m, 1H), 4.31 (d, J=12.0 Hz, 1H), 4.17 (d, J=12.0 Hz, 1H), 4.06 (dd, J=10.4, 7.1 Hz, 1H), 3.97 (dd, J=10.4, 6.4 Hz, 1H), 3.59-3.39 (m, 4H), 3.12-2.96 (m, 3H), 2.86-2.75 (m, 1H), 2.42-2.28 (m, 3H), 2.20-1.93 (m, 3H), 1.91-1.68 (m, 3H), 1.67-1.50 (m, 4H).

### Example 79: 6-(4-(3,8-diazabicyclo[3.2.1]octan-3-yl)-6,8-difluoro-2-(((2R,7aS)-2-fluoro tetrahydro-1H-pyrrolizin-7a(5H)-yl)methoxy)quinazolin-7-yl)-4-methyl-5-(trifluoro methyl) pyridin-2-amine

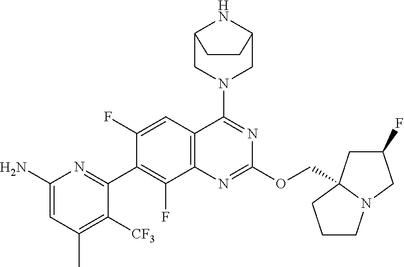 [SMILES: Cc1cc(N)nc(-c2c(F)cc3c(N4CC5CCC(C4)N5)nc(OC[C@@]45CCCN4C[C@H](F)C5)nc3c2F)c1C(F)(F)F]

Synthetic Route

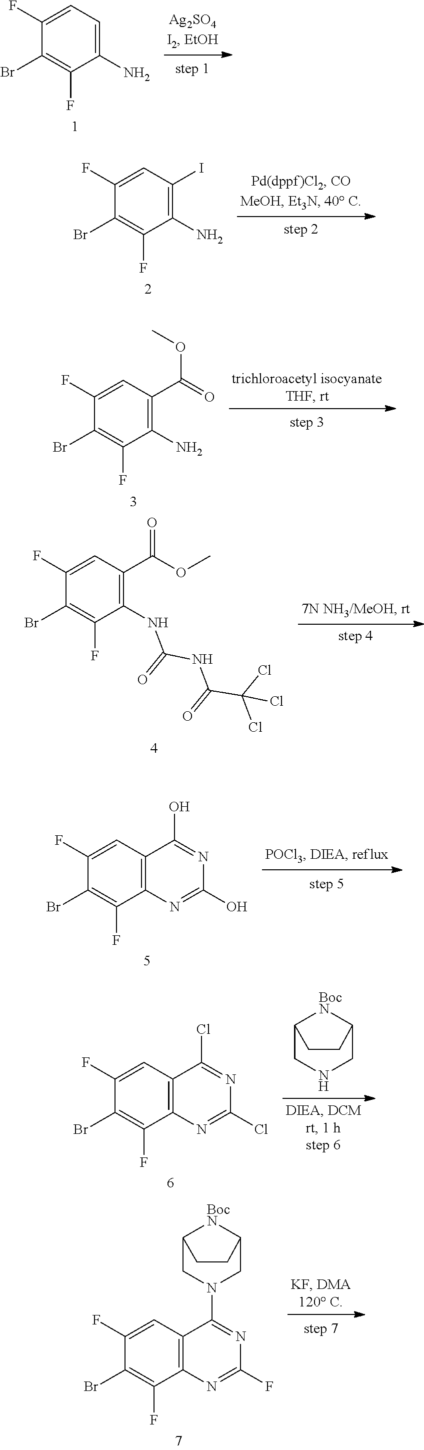

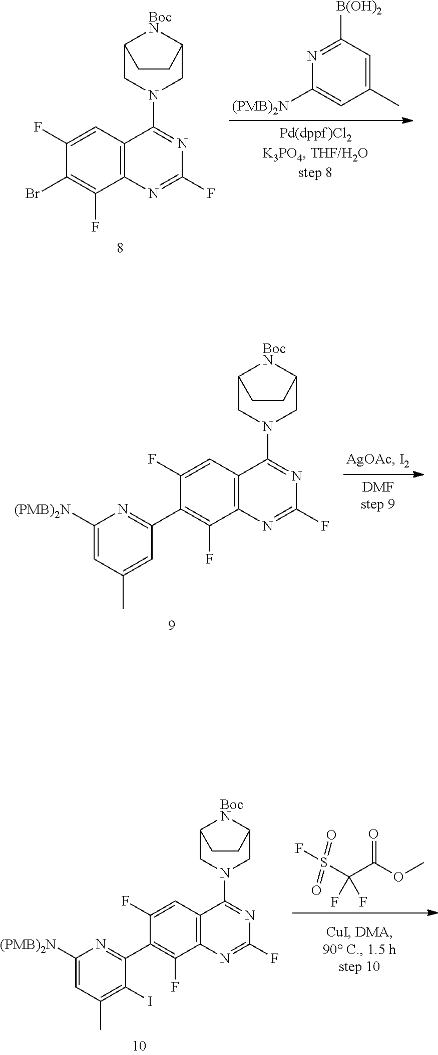

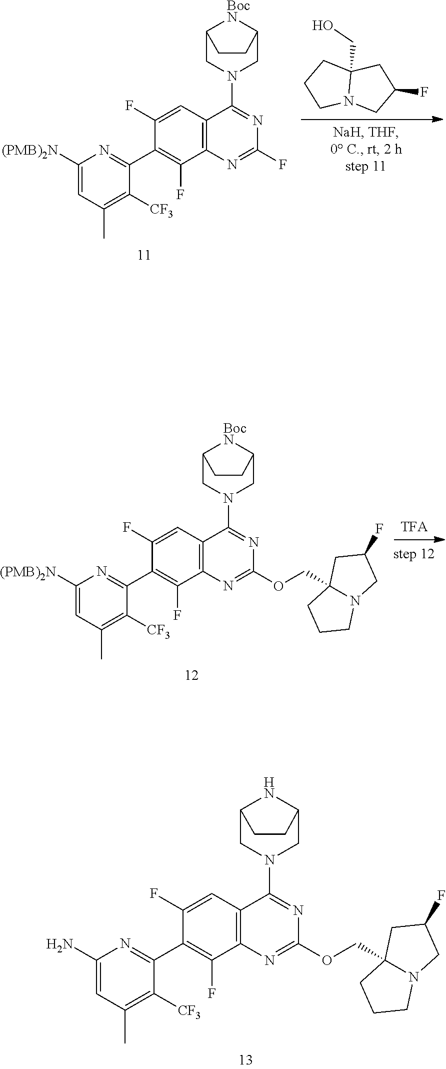

### Step 1: 3-bromo-2,4-difluoro-6-iodoaniline

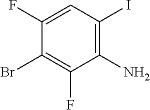 [SMILES: Nc1c(I)cc(F)c(Br)c1F]

A solution of 3-bromo-2,4-difluoroaniline (10 g, 48.07 mmol) in EtOH (300 mL) was added Ag₂SO₄ (14.99 g, 48.07 mmol) and I2 (13.42 g, 52.88 mmol) at 0° C. The mixture was stirred for 2 hrs at rt. The resulting mixture was concentrated under reduced pressure. The residue was purified by combi-flash (PE:EtOAc=3:1) to give the title product (14 g). MS (ESI, m/e) [M+H]⁺ 333.9.

### Step 2: methyl 2-amino-4-bromo-3,5-difluorobenzoate

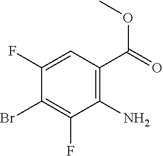

To a stirred mixture of 3-bromo-2,4-difluoro-6-iodoaniline (9 g, 26.95 mmol) and TEA (8.18 g, 80.86 mmol) in MeOH (270 mL) was added Pd(dppf)Cl₂ (3.94 g, 5.39 mmol) in portions at rt. The resulting mixture was stirred overnight at 40° C. under CO atmosphere at 40 psi. The resulting mixture was extracted with EtOAc. The combined organic layers were washed with brine, dried over anhydrous Na₂SO₄. After filtration, the filtrate was concentrated under reduced pressure. The residue was purified by combi-flash (PE/EtOAc=3:1) to give the title product (7 g). MS (ESI, m/e) [M+H]⁺ 266.0.

### Step 3: methyl 4-bromo-3,5-difluoro-2-(3-(2,2,2-trichloroacetyl)ureido)benzoate

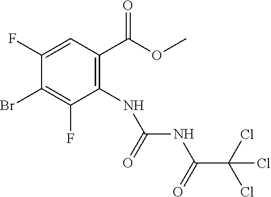 [SMILES: COC(=O)c1cc(F)c(Br)c(F)c1NC(=O)NC(=O)C(Cl)(Cl)Cl]

A mixture of methyl 2-amino-4-bromo-3,5-difluorobenzoate (6 g, 22.55 mmol) and trichloroethanecarbonyl isocyanate (4.67 g, 24.80 mmol) in THF (70 mL) was stirred for 2 hrs at rt. The resulting mixture was extracted with EtOAc. The combined organic layers were washed with brine and dried over anhydrous Na₂SO₄. After filtration, the filtrate was concentrated under reduced pressure. The crude product was used in the next step directly without further purification.

### Step 4: 7-bromo-6,8-difluoroquinazoline-2,4-diol

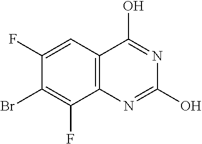 [SMILES: Oc1nc(O)c2cc(F)c(Br)c(F)c2n1]

A mixture of methyl 4-bromo-3,5-difluoro-2-(3-(2,2,2-trichloroacetyl)ureido)benzoate (6 g, 13.20 mmol) in 7 M NH₃ MeOH solution (70 mL) was stirred for 2 hrs at rt. The resulting mixture was concentrated under reduced pressure. The crude product was used in the next step directly without further purification.

### Step 5: 7-bromo-2,4-dichloro-6,8-difluoroquinazoline

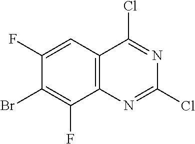 [SMILES: Fc1cc2c(Cl)nc(Cl)nc2c(F)c1Br]

A solution of 7-bromo-6,8-difluoroquinazoline-2,4-diol (6 g, 21.65 mmol) in POCl₃ (60 mL) was added DIEA (6 mL, 34.44 mmol) dropwise portions at 0° C. The resulting mixture was stirred overnight at 120° C. The resulting mixture was concentrated under reduced pressure. The residue was purified by combi-flash (PE/EtOAc=10:1) to give the title product (5.6 g).

### Step 6: tert-butyl 3-(7-bromo-2-chloro-6,8-difluoroquinazolin-4-yl)-3,8-diazabicyclo [3.2.1]octane-8-carboxylate

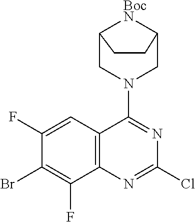 [SMILES: CC(C)(C)OC(=O)N1C2CCC1CN(c1nc(Cl)nc3c(F)c(Br)c(F)cc13)C2]

To a stirred solution of 7-bromo-2,4-dichloro-6,8-difluoroquinazoline (2.6 g, 8.28 mmol) and tert-butyl 3,8-diazabicyclo[3.2.1]octane-8-carboxylate (1.93 g, 9.11 mmol) in DCM (30 mL) was added DIEA (1.07 g, 8.28 mmol) dropwise at rt. The resulting mixture was stirred for 1 h at rt. The resulting mixture was extracted with DCM. The combined organic layers were washed with brine and dried over anhydrous Na₂SO₄. After filtration, the filtrate was concentrated under reduced pressure. The residue was purified by combi-flash (PE/EtOAc=10:1) to give the title product (4 g). MS (ESI, m/e) [M+H]⁺ 491.2.

### Step 7: tert-butyl 3-(7-bromo-2,6,8-trifluoroquinazolin-4-yl)-3,8-diazabicyclo [3.2.1]octane-8-carboxylate

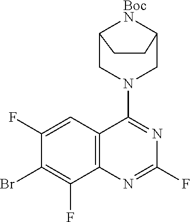 [SMILES: CC(C)(C)OC(=O)N1C2CCC1CN(c1nc(F)nc3c(F)c(Br)c(F)cc13)C2]

To a stirred solution of tert-butyl 3-(7-bromo-2-chloro-6,8-difluoroquinazolin-4-yl)-3,8-diazabicyclo[3.2.1]octane-8-carboxylate (4 g, 8.16 mmol) in DMA (40 mL) was added KF (7.12 g, 122.50 mmol) in portions at rt. The resulting mixture was stirred overnight at 120° C. The resulting mixture was extracted with EtOAc. The combined organic layers were washed with brine and dried over anhydrous Na₂SO₄. After filtration, the filtrate was concentrated under reduced pressure. The residue was purified by combi-flash (PE/EtOAc=10:1) to give the title product (3 g). MS (ESI, m/e) [M+H]⁺ 473.1.

### Step 8: tert-butyl 3-(7-(6-(bis(4-methoxybenzyl)amino)-4-methylpyridin-2-yl)-2,6,8-trifluoroquinazolin-4-yl)-3,8-diazabicyclo[3.2.1]octane-8-carboxylate

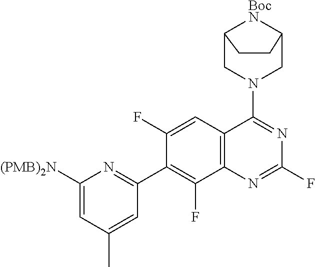

To a stirred solution of tert-butyl 3-(7-bromo-2,6,8-trifluoroquinazolin-4-yl)-3,8-diaza bicyclo[3.2.1]octane-8-carboxylate (500 mg, 1.05 mmol) and 6-(bis((4-methoxyphenyl) methyl)amino)-4-methylpyridin-2-ylboronic acid (1.66 g, 4.22 mmol) in THF (10 mL) and H₂O (2 mL) were added K₃PO₄ (449 mg, 2.11 mmol) and Pd(dppf)Cl₂ (309 mg, 0.42 mmol) in portions at room temperature. The resulting mixture was stirred for 2 hrs at 65° C. The resulting mixture was extracted with EtOAc. The combined organic layers were washed with brine and dried over anhydrous Na₂SO₄. After filtration, the filtrate was concentrated under reduced pressure. The residue was purified by combi-flash (PE/EtOAc=3:1) to give the title product (775 mg). MS (ESI, m/e) [M+H]⁺ 741.3.

### Step 9: tert-butyl 3-(7-(6-(bis(4-methoxybenzyl)amino)-3-iodo-4-methylpyridin-2-yl)-2,6,8-trifluoroquinazolin-4-yl-3,8-diazabicyclo[3.2.1]octane-8-carboxylate

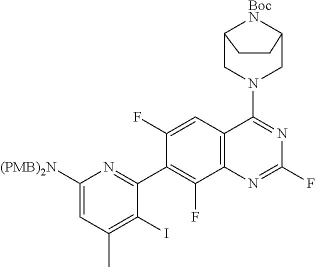

A solution of tert-butyl 3-(7-(6-(bis((4-methoxyphenyl) methyl)amino)-4-methylpyridin-2-yl)-2,6,8-trifluoroquinazolin-4-yl)-3,8-diazabicyclo[3.2.1] octane-8-carboxylate (775 mg, 1.04 mmol) in DMF (10 mL) was added silver acetate (437 mg, 2.61 mmol) and I₂ (797 mg, 3.13 mmol) in portions at rt. The resulting mixture was stirred for 2 hrs at rt. The reaction was quenched with Na₂S₂O₃ aqueous solution at 0° C. The resulting mixture was extracted with EtOAc. The combined organic layers were washed with brine and dried over anhydrous Na₂SO₄. After filtration, the filtrate was concentrated under reduced pressure. The residue was purified by combi-flash (PE/EtOAc=3:1) to give the title product (750 mg). MS (ESI, m/e) [M+H]⁺ 867.4.

### Step 10: tert-butyl 3-(7-(6-(bis(4-methoxybenzyl)amino)-4-methyl-3-(trifluoro methyl) pyridin-2-yl)-2,6,8-trifluoroquinazolin-4-yl)-3,8-diazabicyclo[3.2.1]octane-8-carboxylate

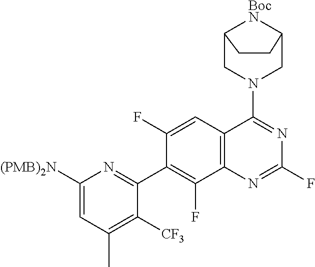

A mixture of tert-butyl 3-(7-(6-(bis(4-methoxy benzyl)amino)-3-iodo-4-methyl pyridin-2-yl)-2,6,8-trifluoroquinazolin-4-yl)-3,8-diazabicyclo [3.2.1]octane-8-carboxylate (200 mg, 0.23 mmol) and CuI (175.8 mg, 0.92 mmol) in DMA was stirred for 15 min at rt. To the above mixture was added methyl 2,2-difluoro-2-sulfoacetate (443 mg, 2.31 mmol) dropwise portions at rt. The resulting mixture was stirred for additional 90 min at 90° C. The resulting mixture was extracted with EtOAc. The combined organic layers were washed with brine and dried over anhydrous Na₂SO₄. After filtration, the filtrate was concentrated under reduced pressure. The residue was purified by combi-flash (PE/EtOAc=3:1) to give the title product (180 mg). MS (ESI, m/e) [M+H]⁺ 809.4.

### Step 11: tert-butyl 3-(7-(6-(bis(4-methoxybenzyl)amino)-4-methyl-3-(trifluoro methyl) pyridin-2-yl)-6,8-difluoro-2-(((2R,7aS)-2-fluorotetrahydro-1H-pyrrolizin-7a(5H)-yl) methoxy) quinazolin-4-yl)-3,8-diazabicyclo[3.2.1]octane-8-carboxylate

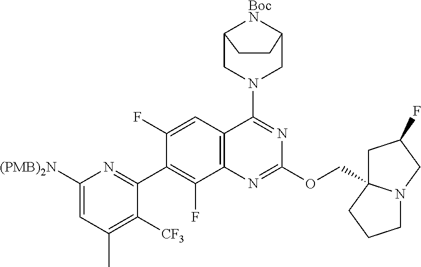

To a solution of tert-butyl 3-(7-(6-(bis(4-methoxy benzyl)amino)-4-methyl-3-(trifluoro methyl)pyridin-2-yl)-2,6,8-trifluoroquinazolin-4-yl)-3,8-diazabicyclo[3.2.1]octane-8-carboxylate (180 mg, 0.22 mmol) and ((2R,7aS)-2-fluoro-hexahydropyrrolizin-7a-yl)methanol (43 mg, 0.26 mmol) in THF (4.5 mL) was added sodium hydride (60 wt % in oil, 11 mg, 0.26 mmol) at 0° C. The mixture was stirred for 1 h at rt. The reaction was quenched with water/ice at 0° C. The resulting mixture was extracted with EtOAc. The combined organic layers were washed with brine and dried over anhydrous Na₂SO₄. After filtration, the filtrate was concentrated under reduced pressure. The residue was purified by combi-flash (PE/EtOAc=1:1) to give the title product (170 mg). MS (ESI, m/e) [M+H]⁺ 948.5.

### Step 12: 6-(4-(3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-4-methyl-5-(trifluoro methyl)pyridin-2-amine

A mixture of tert-butyl 3-(7-(6-(bis(4-methoxybenzyl)amino)-4-methyl-3-(trifluoro methyl)pyridin-2-yl)-6,8-difluoro-2-(((2R,7aS)-2-fluorotetrahydro-1H-pyrrolizin-7a(5H)-yl) methoxy)quinazolin-4-yl)-3,8-diazabicyclo[3.2.1]octane-8-carboxylate (170 mg, 0.17 mmol) in TFA (5 mL) was stirred for 3 h at 50° C. The resulting mixture was concentrated under reduced pressure. The crude product was purified by Chiral-Prep-HPLC to afford the title product (48 mg). MS (ESI, m/e) [M+H]⁺ 608.2. ¹H NMR (400 MHz, CDCl₃) δ 7.30 (s, 1H), 6.55-6.40 (m, 1H), 5.54-5.29 (m, 1H), 5.00-4.80 (m, 1H), 4.60-4.30 (m, 4H), 4.10-3.80 (m, 3H), 3.80-3.60 (m, 2H), 3.50-3.30 (m, 1H), 3.20-3.00 (m, 1H), 2.60-2.25 (m, 6H), 2.20-2.00 (m, 6H), 1.95-1.90 (m, 1H).

### Example 80: 3-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-5-chloro-4-(trifluoro methyl)aniline

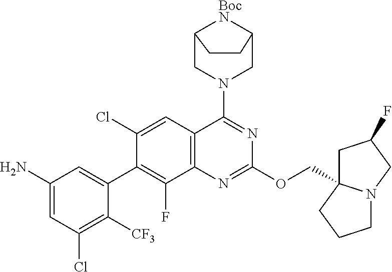 [SMILES: CC(C)(C)OC(=O)N1C2CCC1CN(c1nc(OC[C@@]34CCCN3C[C@H](F)C4)nc3c(F)c(-c4cc(N)cc(Cl)c4C(F)(F)F)c(Cl)cc13)C2]

Synthetic Route

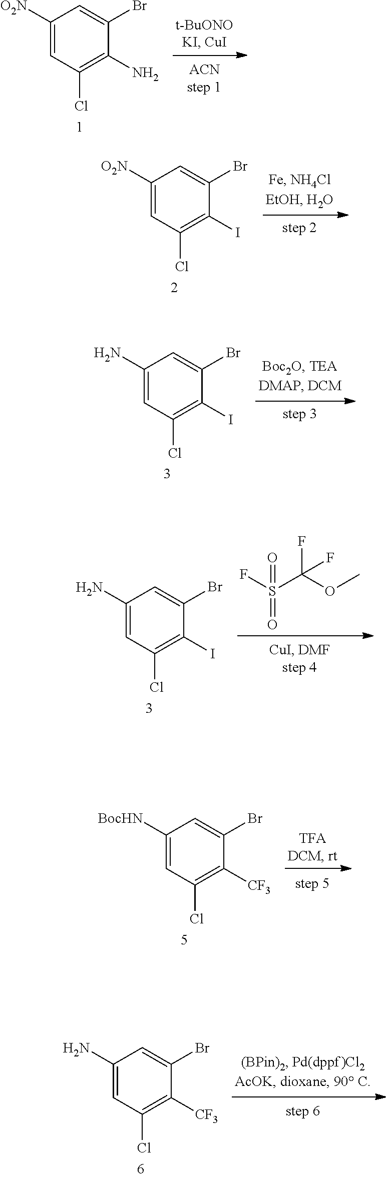

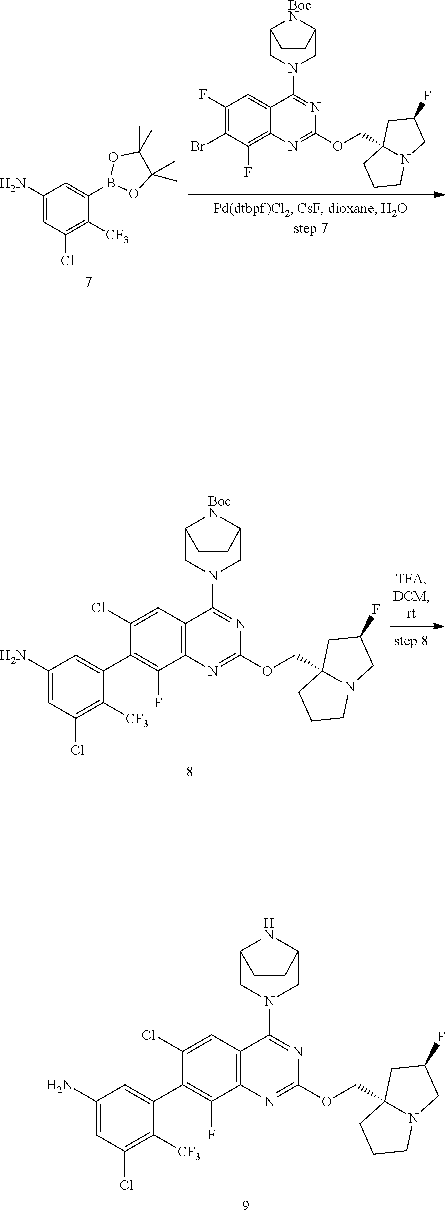

### Step 1: 1-bromo-3-chloro-2-iodo-5-nitrobenzene

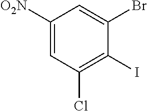 [SMILES: O=[N+]([O-])c1cc(Cl)c(I)c(Br)c1]

In a 250 mL round bottom ask was added 2-bromo-6-chloro-4-nitroaniline (5.0 g, 19.88 mmol), ^{t}BuONO (11.83 g, 114.72 mmol), CuI (4.56 g, 24 mmol), KI (16.50 g, 99.42 mol) and MeCN (50 mL). The reaction was stirred at 80° C. for 12 hrs. The reaction mixture was filtered. The filtrate was concentrated under vacuum. The residue was dissolved in EtOAc (200 mL). EtOAc phase was washed by saturated Na₂S₂O₃ (50 mL), water (50 mL) and brine (50 mL). EtOAc phase was dried over anhydrous Na₂SO₄, and then evaporated under vacuum. The resulting residue was applied on to a silica-gel column and eluted with PE:EtOAc=12:1 to afford the title product (6.20 g) which was used directly in the next step.

### Step 2: 3-bromo-5-chloro-4-iodoaniline

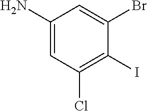

In a 250 mL round bottom flask was added 1-bromo-3-chloro-2-iodo-5-nitrobenzene (1.0 g, 2.76 mmol), Fe (1.55 g, 27.60 mmol), NH₄Cl (1.48 g, 27.66 mmol), EtOH (20 mL) and water (10 mL). The reaction was stirred at 100° C. for 1 h. The reaction mixture was filtered. The filtrate was concentrated under vacuum to remove EtOH. The resulting mixture was diluted by water (100 mL). The solution was extracted by EtOAc (100 mL×3). Organic layer was dried over anhydrous Na₂SO₄ and concentrated under vacuum. This affords the title product (871 mg). MS (ESI, m/e) [M+H]⁺ 331.7, 333.7.

### Step 3: tert-butyl (3-bromo-5-chloro-4-iodophenyl)carbamate

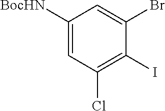 [SMILES: CC(C)(C)OC(=O)Nc1cc(Cl)c(I)c(Br)c1]

Into a 250 mL round bottom flask was added 3-bromo-5-chloro-4-iodoaniline (1.0 g, 3.00 mmol), DCM (10 mL), Boc₂O (985 mg, 4.5 mmol), TEA (456 mg, 4.5 mmol) and DMAP (37 mg, 0.3 mmol). The reaction was stirred at 50° C. overnight. The reaction mixture was concentrated under vacuum. The resulting residue was applied into a silica-gel column and eluted with EtOAc/PE=0-6% in 15 min to give the title product (850 mg) which was used directly in the next step.

Step 4: tert-butyl 3-bromo-5-chloro-4-(trifluoromethyl)phenyl)carbamate

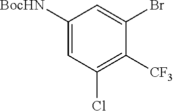 [SMILES: CC(C)(C)OC(=O)Nc1cc(Cl)c(C(F)(F)F)c(Br)c1]

In a 100 mL round bottom flask was added tert-butyl (3-bromo-5-chloro-4-(trifluoro methyl)phenyl)carbamate (750 mg, 1.73 mmol), dimethylformamide (20 mL), methyl 2,2-difluoro-2-sulfoacetate (1.99 g, 12.13 mmol) and CuI (1.98 g, 10.40 mmol). The reaction was stirred at 90° C. for 3 hrs. The reaction mixture was filtered. The filtrate was diluted by EtOAc (100 mL) and washed by brine (50 mL×3). Organic phase was dried over anhydrous Na₂SO₄ and was then concentrated under vacuum. The residue was purified by TLC with PE:EtOAc=3:1 (Rf=0.6) to afford the title product (400 mg) which was used directly in the next step.

### Step 5: 3-bromo-5-chloro-4-(trifluoromethyl)aniline

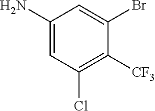 [SMILES: Nc1cc(Cl)c(C(F)(F)F)c(Br)c1]

Into a 25 ml round bottom flask was added tert-butyl (3-bromo-5-chloro-4-(trifluoro methyl)phenyl)carbamate (200 mg, 0.53 mmol), TFA (3 mL) and DCM (3 mL). The reaction mixture was stirred for 2 hrs at rt. The reaction mixture was concentrated under vacuum. This afforded the title product (133 mg). MS (ESI, m/e) [M+MeCN]⁺ 315, 317.

### Step 6: 3-chloro-5-(4,4,5,5-tetramethyl-1,3,2-dioxaborolan-2-yl)-4-(trifluoromethyl) aniline

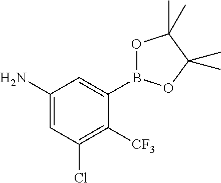 [SMILES: CC1(C)OB(c2cc(N)cc(Cl)c2C(F)(F)F)OC1(C)C]

In a 100 mL round bottom flask was added 3-bromo-5-chloro-4-(trifluoromethyl)aniline (1.0 g, 3.64 mmol), bis(pinacolato)diboron (1.02 g, 4.00 mmol), Pd(dppf)Cl₂.DCM (0.13 g, 0.18 mmol), AcOK (1.07 g, 10.93 mmol) and dioxane (50 mL). The reaction was stirred at 90° C. overnight. The reaction mixture was filtered. The filtrate was concentrated under vacuum. The residue was dissolved in EtOAc (100 mL) and washed with brine (30 mL×5). The organic layer was dried by anhydrous Na₂SO₄ and was then concentrated under vacuum. The residue was applied on to a silica-gel column and eluted with EtOAc/PE=0%-12% in 20 min to afford the title product (832 mg). MS (ESI, m/e) [M+H]⁺ 322.0.

### Step 7: tert-butyl 3-(7-(5-amino-3-chloro-2-(trifluoromethyl)phenyl)-6-chloro-8-fluoro-2-(((2R,7aS)-2-fluorotetrahydro-1H-pyrrolizin-7a(5H)-yl)methoxy)quinazolin-4-yl)-3,8-diaza bicyclo[3.2.1]octane-8-carboxylate

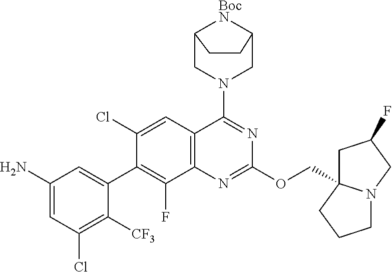 [SMILES: CC(C)(C)OC(=O)N1C2CCC1CN(c1nc(OC[C@@]34CCCN3C[C@H](F)C4)nc3c(F)c(-c4cc(N)cc(Cl)c4C(F)(F)F)c(Cl)cc13)C2]

To a 25 mL round bottom flask was added 3-chloro-5-(4,4,5,5-tetramethyl-1,3,2-dioxaborolan-2-yl)-4-(trifluoromethyl)aniline (153 mg, 0.47 mmol), tert-butyl 3-(2-(((2R,7aS)-2-fluoro-hexahydropyrrolizin-7a-yl)methoxy)-7-bromo-6-chloro-8-fluoroquinazolin-4-yl)-3,8-diazabicyclo[3.2.1]octane-8-carboxylate (100 mg, 0.16 mmol), dioxane (5 mL), H₂O (1 mL), CsF (72.5 mg, 0.48 mmol) and Pd(dtbpf)Cl₂ (20.7 mg, 0.03 mmol). The reaction was stirred at 90° C. for 12 hrs. The reaction mixture was filtered. The filtrate was diluted with EtOAc (100 mL) and washed with brine (30 ml×3). EA phase was dried over anhydrous Na₂SO₄, and then concentrated under vacuum. The residue was purified by TLC with DCM:MeOH=10:1 (Rf=0.4) to afford the title product (52 mg). MS (ESI, m/e) [M+H]⁺ 743.5.

### Step 8: 6-(4-(3,8-diazabicyclo[3.2.1]octan-3-yl)-6-chloro-8-fluoro-2-(((2R,7aS)-2-fluoro tetrahydro-1H-pyrrolizin-7a(5H)-yl)methoxy)quinazolin-7-yl)-3-fluoro-4-methyl-5-(trifluoro methyl)pyridin-2-amine

To a 25 ml round bottom flask was added tert-butyl 3-(2-(((2R,7aS)-2-fluoro-hexahydro pyrrolizin-7a-yl)methoxy)-7-(5-amino-3-chloro-2-(trifluoromethyl)phenyl)-6-chloro-8-fluoro quinazolin-4-yl)-3,8-diazabicyclo[3.2.1]octane-8-carboxylate (52 mg, 0.070 mmol), DCM (2 mL) and TFA (2 mL). The reaction was stirred at rt for 4 hrs. The reaction was concentrated under vacuum. The crude product (80 mg) was purified by Prep-HPLC to afford the title product (2.7 mg, TFA salt). MS (ESI, m/e) [M+H]⁺ 643.3. ¹H NMR (300 MHz, DMSO-d₆) δ 7.84 (s, 1H), 6.88 (d, J=2.5 Hz, 1H), 6.33 (d, J=6.4 Hz, 2H), 5.26 (d, J=54.1 Hz, 1H), 4.56-4.14 (m, 2H), 4.12-3.84 (m, 2H), 3.55 (m, 4H), 3.08 (m, 2H), 2.98 (m, 1H), 2.81 (m, 1H), 2.12 (m, 1H), 2.02 (m, 2H), 1.76 (m, 3H), 1.63 (m, 3H), 1.13 (m, 1H).

### Example 81: 2-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-3-fluorophenol

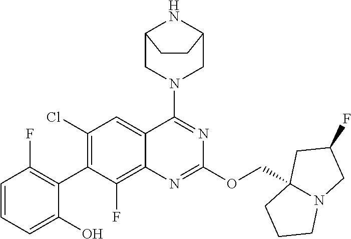 [SMILES: Oc1cccc(F)c1-c1c(Cl)cc2c(N3CC4CCC(C3)N4)nc(OC[C@@]34CCCN3C[C@H](F)C4)nc2c1F]

Synthetic Route:

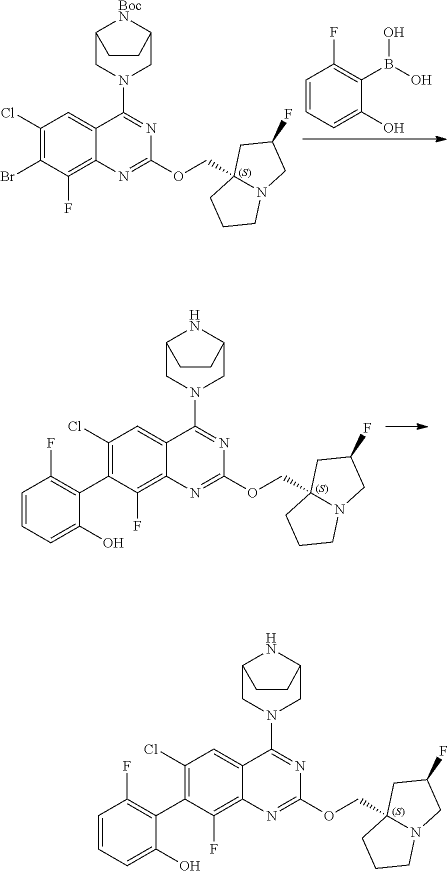

### Step 1: tert-butyl 3-(6-chloro-8-fluoro-7-(2-fluoro-6-hydroxyphenyl)-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

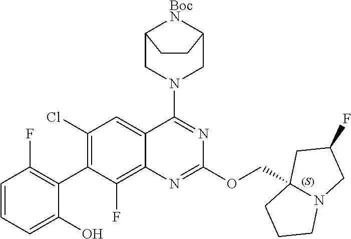

The mixture of tert-butyl 3-(7-bromo-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (126 mg, 0.2 mmol), (2-fluoro-6-hydroxyphenyl)boronic acid (94.7 mg, 0.6 mmol) and Pd₂(dba)₃ (36 mg, 0.04 mmol) (R)-Antphos (30 mg, 0.08 mmol) and K₃PO₄ (127 mg, 0.6 mmol) in toluene (5 mL) and H₂O (1 mL) was stirred at 100° C. for 5 hrs. Then it was added Pd-Ruphos-G3 (33 mg, 0.04 mmol), Ruphos (36 mg, 0.08 mmol), and the mixture was stirred at 100° C. for 15 hrs. After completion. The reaction mixture was purified by Prep-TLC (DCM:MeOH=10:1) to give the titled product (50 mg). MS (ESI, m/e) [M+1]⁺660.

### Step 2: 2-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-3-fluorophenol, formic acid salt

To a solution of tert-butyl 3-(6-chloro-8-fluoro-7-(2-fluoro-6-hydroxyphenyl)-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (50 mg, 0.0759 mmol) in DCM (5 mL) was added TFA (1 mL) at room temperature. The resulting mixture was stirred at room temperature for 3 hrs. After completion, the reaction mixture was concentrated and the residue was purified by prep-HPLC to give the title product (8.0 mg). ¹H NMR (500 MHz, CD₃OD) δ 8.37 (s, 1H), 7.92 (s, 1H), 7.35-7.31 (m, 1H), 6.77 (d, J=8.3 Hz, 1H), 6.72 (t, J=8.7 Hz, 1H), 5.52-5.42 (m, 1H), 4.68-4.43 (m, 4H), 4.01 (s, 2H), 3.82-3.56 (m, 5H), 3.33-3.28 (m, 1H), 2.61-2.38 (m, 2H), 2.35-2.30 (m, 1H), 2.28-2.18 (m, 2H), 2.13-1.95 (m, 5H). MS (ESI, m/e) [M+1]⁺560.4.

### Example 82: 4-(4-(3,8-diazabicyclo[3.2.1]octan-3-yl)-8-chloro-2-(((2R,7aS)-2-fluorotetrahydro-1H-pyrrolizin-7a(5H)-yl)methoxy)-6-(trifluoromethyl)quinazolin-7-yl)-7-chlorobenzo[d]thiazol-2-amine

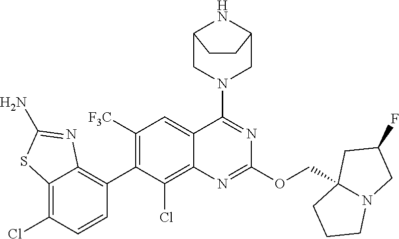 [SMILES: Nc1nc2c(-c3c(C(F)(F)F)cc4c(N5CC6CCC(C5)N6)nc(OC[C@@]56CCCN5C[C@H](F)C6)nc4c3Cl)ccc(Cl)c2s1]

Synthetic Route:

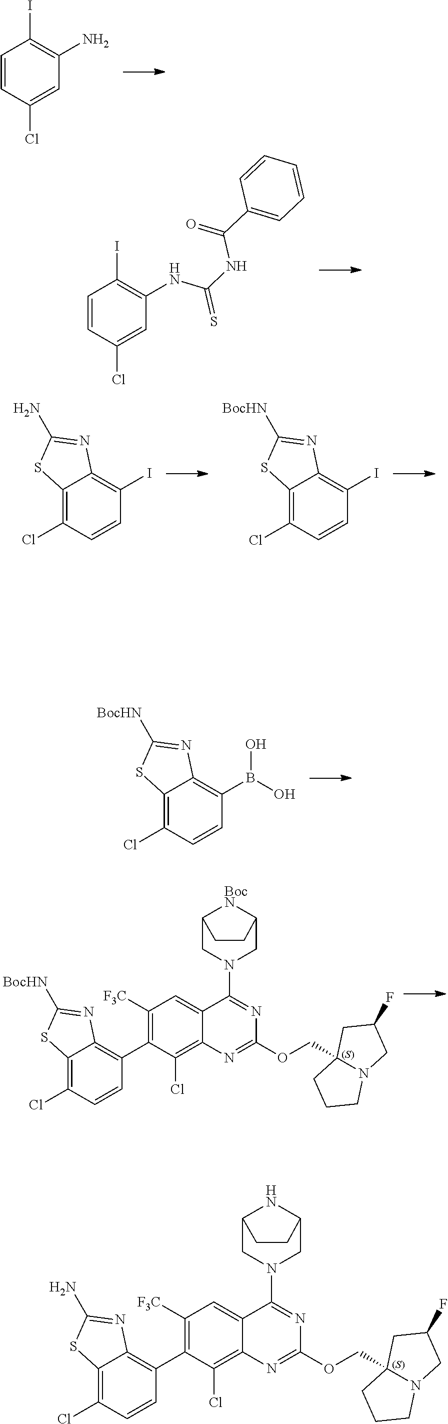

### Intermediate 82a: tert-butyl 3-(7-bromo-8-chloro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate

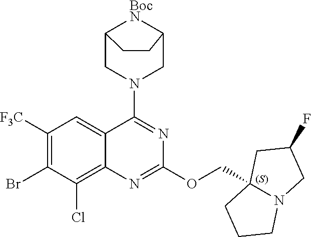

To a solution of ((2R,7aS)-2-fluorotetrahydro-1H-pyrrolizin-7a(5H)-yl)methanol (318 mg, 2 mmol) in THF (5 mL) was added sodium hydride (96 mg, 4 mmol) at room temperature. The resulting mixture was stirred at room temperature for 1 h, then tert-butyl 3-(7-bromo-8-chloro-2-fluoro-6-(trifluoromethyl)quinazolin-4-yl)-3,8-diazabicyclo[3.2.1]octane-8-carboxylate (1080 mg, 2 mmol) was added to reaction mixture and stirred for 2 hrs at room temperature. After completion. The reaction mixture was purified by silica gel column chromatography, eluted with EtOAc to give the title product (1100 mg). MS (ESI, m/e) [M+1]⁺678.3, 680.3.

### Step 1: N-((5-chloro-2-iodophenyl)carbamothioyl)benzamide

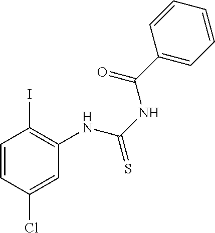 [SMILES: O=C(NC(=S)Nc1cc(Cl)ccc1I)c1ccccc1]

To a solution of 5-chloro-2-iodoaniline (7590 mg, 30 mmol) in THF (60 mL) was added benzoyl isothiocyanate (4890 mg, 30 mmol) at room temperature. The resulting mixture was stirred at room temperature for 16 hrs. After completion, the reaction mixture was concentrated to give the title product (12.5 g). MS (ESI, m/e) [M+1]⁺417.

### Step 2: 7-chloro-4-iodobenzo[d]thiazol-2-amine

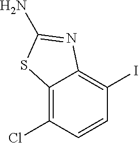 [SMILES: Nc1nc2c(I)ccc(Cl)c2s1]

To a solution of N-((5-chloro-2-iodophenyl)carbamothioyl)benzamide (12.0 g, 28.8 mmol) in conc. H₂SO₄ (50 mL) was added NaBr (595 mg, 5.77 mmol) at 0° C. The resulting mixture was heated at 80° C. for 5 hrs. After completion, the mixture was decanted into ice-water, adjusted pH to 9 with 12N NaOH solution, filtered, the solid was washed with water to give 3.5 g of product. MS (ESI, m/e) [M+1]⁺311.

### Step 3: tert-butyl (7-chloro-4-iodobenzo[d]thiazol-2-yl)carbamate

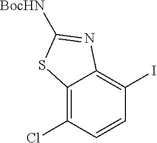 [SMILES: CC(C)(C)OC(=O)Nc1nc2c(I)ccc(Cl)c2s1]

To a solution of 7-chloro-4-iodobenzo[d]thiazol-2-amine (3100 mg, 10 mmol), Boc₂O (2180 mg, 10 mmol), DIEA (2580 mg, 20 mmol) and DMAP (61 mg, 0.5 mmol) in DCM (50 mL) was stirred at room temperature for 2 hrs. After completion. The reaction mixture was added water, extracted with DCM, the combined organic layer was dried over Na₂SO₄, filtered, concentrated and purified by silica gel (PE:EtOAc=4:1) to give the title product (3.8 g). MS (ESI, m/e) [M−55]⁺355.

### Step 4: (2-((tert-butoxycarbonyl)amino)-7-chlorobenzo[d]thiazol-4-yl)boronic acid

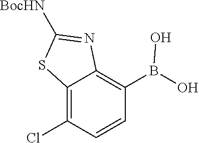 [SMILES: CC(C)(C)OC(=O)Nc1nc2c(B(O)O)ccc(Cl)c2s1]

The mixture of tert-butyl (7-chloro-4-iodobenzo[d]thiazol-2-yl)carbamate (520 mg, 1.27 mmol), B₂Pin₂ (3.22 g, 12.7 mmol), PdCl₂(dppf)₂DCM (103 mg, 0.127 mmol) and KOAc (380 mg, 3.8 mmol) in dioxane (50 mL) was stirred at 100° C. for 15 hrs. After completion. The reaction mixture was purified by silica gel (PE:EtOAc=3:1) to give the title product (200 mg, 48%), MS (ESI, m/e) [M+1]⁺329

### Step 5: tert-butyl 3-(7-(2-((tert-butoxycarbonyl)amino)-7-chlorobenzo[d]thiazol-4-yl)-8-chloro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate

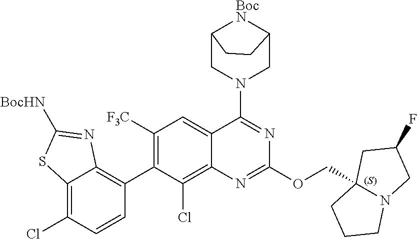

The mixture of tert-butyl 3-(7-bromo-8-chloro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate (226 mg, 0.333 mmol), (2-((tert-butoxycarbonyl)amino)-7-chlorobenzo[d]thiazol-4-yl)boronic acid (410 mg, 1.0 mmol) and Pd₂(dba)₃ (61 mg, 0.0666 mmol), (R)-Antphos (49 mg, 0.133 mmol) and K₃PO₄ (212 mg, 1.0 mmol) in toluene (10 mL) and H₂O (2 mL) was stirred at 110° C. for 5 hrs. After completion, the reaction mixture was purified by Prep-TLC (PE:EtOAc=1:2) to give the title product (200 mg). MS (ESI, m/e) [M+1]⁺882.

### Step 6: 4-(4-(3,8-diazabicyclo[3.2.1]octan-3-yl)-8-chloro-2-(((2R,7aS)-2-fluorotetrahydro-1H-pyrrolizin-7a(5H)-yl)methoxy)-6-(trifluoromethyl)quinazolin-7-yl)-7-chlorobenzo[d]thiazol-2-amine, formic acid salt

To a solution of tert-butyl 3-(7-(2-((tert-butoxycarbonyl)amino)-7-chlorobenzo[d]thiazol-4-yl)-8-chloro-2-((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate (200 mg, 0.227 mmol) in DCM (10 mL) was added TFA (3 mL) at room temperature. The resulting mixture was stirred at room temperature for 4 hrs. After completion. The reaction mixture was concentrated to give the residue. The residue was purified by Prep-HPLC to give the title product (45.0 mg). ¹H NMR (500 MHz, CD₃OD) δ 8.48 (s, 1H), 8.27 (s, 1H), 7.20 (d, J=8.1 Hz, 1H), 7.10 (d, J=8.1 Hz, 1H), 5.46-5.36 (m, 1H), 4.64-4.62 (m 1H), 4.55-4.52 (m, 2H), 4.46-4.42 (m, 1H), 3.89 (s, 2H), 3.83-3.81 (m, 1H), 3.76-3.74 (m, 1H), 3.68-3.41 (m, 3H), 3.32-3.20 (m, 1H), 2.56-2.36 (m, 2H), 2.33-2.22 (m, 1H), 2.18-2.12 (m, 2H), 2.05-1.79 (m, 5H). MS (ESI, m/e) [M+1]⁺682.4.

### Example 83: 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-chlorobenzo[d]thiazol-2-amine

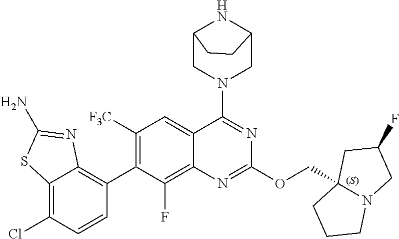

Synthetic Route:

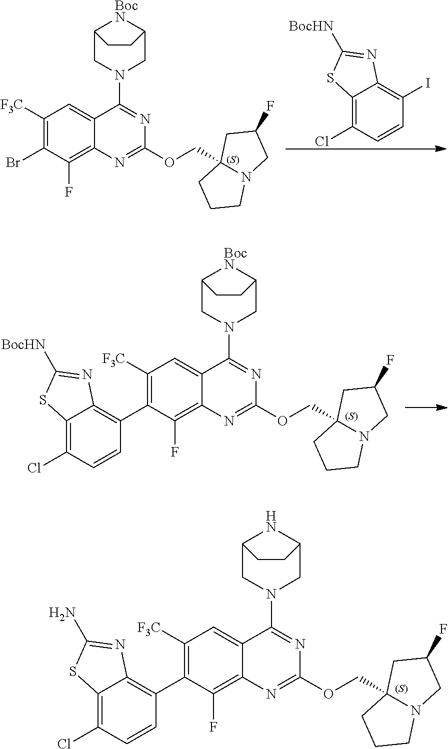

### Step 1: tert-butyl 3-(7-(2-((tert-butoxycarbonyl)amino)-7-chlorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate

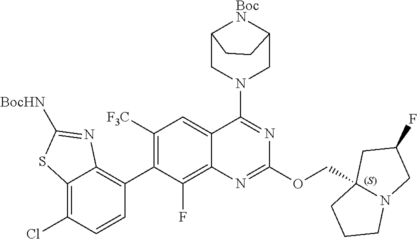

The mixture of tert-butyl 3-(7-bromo-8-fluoro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate (100 mg, 0.15 mmol), (2-((tert-butoxycarbonyl)amino)-7-chlorobenzo[d]thiazol-4-yl)boronic acid (123 mg, 0.3 mmol) and Pd-Ruphos-G3 (12 mg, 0.015 mmol), Ruphos (15 mg, 0.03 mmol), K₃CO₃ (63 mg, 0.45 mmol) in dioxane (5 mL) and H₂O (1 mL) was stirred at 100° C. for 15 hrs. After completion, the reaction mixture was purified by Prep-TLC (PE:EtOAc=1:3) to give the title product (100 mg, crude). MS (ESI, m/e) [M+1]⁺866.

### Step 2: 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-chlorobenzo[d]thiazol-2-amine, formic acid salt

To a solution of tert-butyl 3-(7-(2-((tert-butoxycarbonyl)amino)-7-chlorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate (100 mg, 0.116 mmol) in DCM (4 mL) was added TFA (2 mL) at room temperature. The resulting mixture was stirred at room temperature for 4 hrs. After completion, the reaction mixture was concentrated and the residue was purified by Prep-HPLC to give the title product (1.1 mg). ¹H NMR (500 MHz, CD₃OD) δ 8.44 (s, 1H), 8.15 (s, 1H), 7.21-7.19 (d, J=8.1 Hz, 1H), 7.19-7.17 (d, J=8.1 Hz, 1H), 5.47-5.37 (m, 1H), 4.72-4.43 (m, 5H), 3.88 (s, 2H), 3.81-3.76 (m, 2H), 3.71-3.51 (m, 3H), 2.49-2.35 (m, 2H), 2.31-2.26 (m, 1H), 2.20-2.11 (m, 2H), 2.06-1.85 (m, 5H). MS (ESI, m/e) [M+1]⁺666.2.

### Example 84: 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-5,7-difluorobenzo[d]thiazol-2-amine

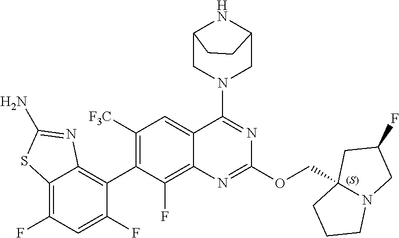

Synthetic Route:

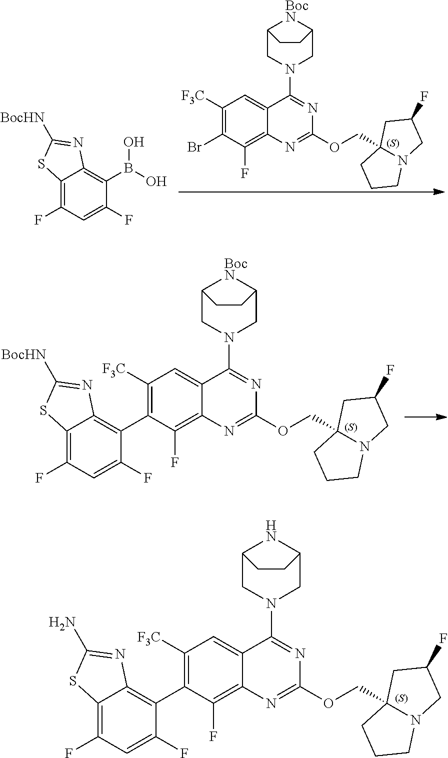

### Step 1: tert-butyl 3-(7-(2-((tert-butoxycarbonyl)amino)-5,7-difluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-38-diazabicyclo[3.2.1]octane-8-carboxylate

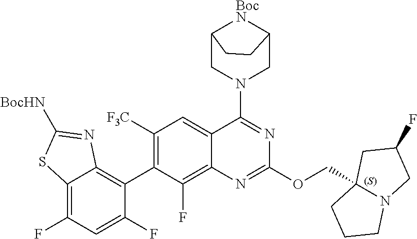

The mixture of tert-butyl 3-(7-bromo-8-fluoro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate (120 mg, 0.181 mmol), (2-((tert-butoxycarbonyl)amino)-5,7-difluorobenzo[d]thiazol-4-yl)boronic acid (120 mg, 0.362 mmol), Pd₂(dba)₃ (33 mg, 0.0362 mmol), (R)-Antphos (27 mg, 0.0724 mmol) and K₃PO₄ (120 mg, 0.566 mmol) in toluene (10 mL) and H₂O (2 mL) was stirred at 110° C. for 5 hrs. After completion. The reaction mixture was purified by Prep-TLC (PE:EtOAc=1:2) to give the title product (30 mg). MS (ESI, m/e) [M+1]⁺868.

### Step 2: 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-5,7-difluorobenzo[d]thiazol-2-amine, formic acid salt

To a solution of tert-butyl 3-(7-(2-((tert-butoxycarbonyl)amino)-5,7-difluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate (30 mg, 0.0346 mmol) in DCM (10 mL) was added TFA (3 mL) at room temperature. The resulting mixture was stirred at room temperature for 4 hrs. After completion, the reaction mixture was concentrated and the residue was purified by prep-HPLC to give the title product (3.45 mg). ¹H NMR (500 MHz, CD₃OD) δ 8.42 (s, 1H), 8.17 (s, 1H), 6.90 (t, J=9.7 Hz, 1H), 5.50-5.39 (m, 1H), 4.72-4.57 (m, 2H), 4.56-4.39 (m, 2H), 3.96 (s, 2H), 3.87-3.77 (m, 2H), 3.75-3.51 (m, 3H), 3.29-3.23 (m, 1H), 2.60-2.36 (m, 2H), 2.30-2.26 (m, 1H), 2.18-2.10 (m, 2H), 2.09-1.90 (m, 5H). MS (ESI, m/e) [M+1]⁺668.2.

### Example 85: 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-6,7-difluorobenzo[d]thiazol-2-amine

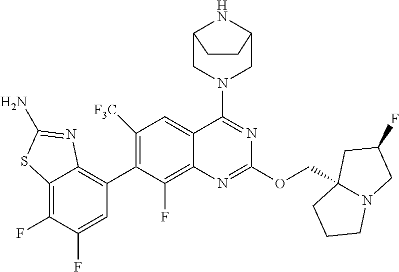 [SMILES: Nc1nc2c(-c3c(C(F)(F)F)cc4c(N5CC6CCC(C5)N6)nc(OC[C@@]56CCCN5C[C@H](F)C6)nc4c3F)cc(F)c(F)c2s1]

Synthetic Route

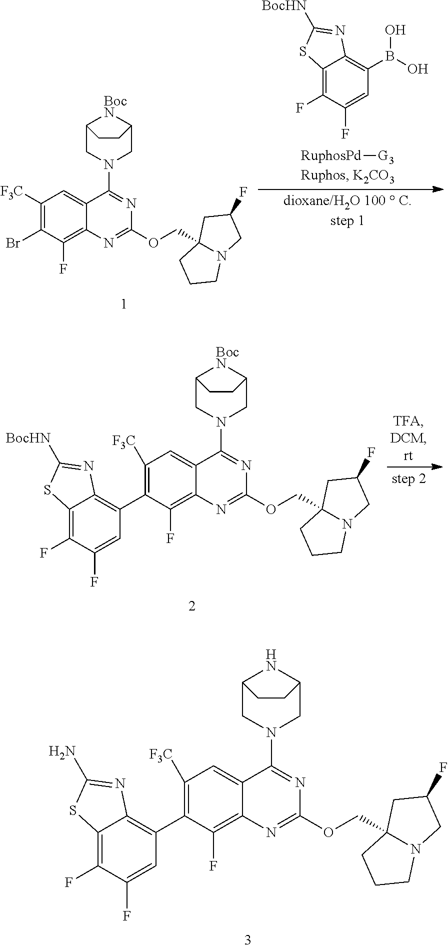

### Step 1: tert-butyl 3-(7-(2-((tert-butoxycarbonyl)amino)-6,7-difluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3,8-diazabicyclo [3.2.1]octane-8-carboxylate

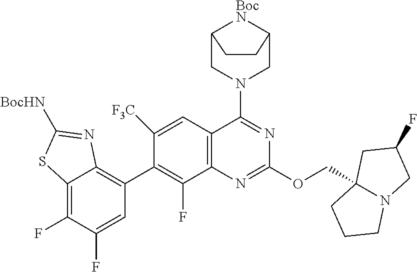 [SMILES: CC(C)(C)OC(=O)Nc1nc2c(-c3c(C(F)(F)F)cc4c(N5CC6CCC(C5)N6C(=O)OC(C)(C)C)nc(OC[C@@]56CCCN5C[C@H](F)C6)nc4c3F)cc(F)c(F)c2s1]

To a solution of tert-butyl 3-(7-bromo-8-fluoro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate (108 mg, 0.16 mmol) in dioxane/H₂O (20 mL+6 mL) was subsequentially added (2-((tert-butoxycarbonyl)amino)-6,7-difluorobenzo[d]thiazol-4-yl)boronic acid (121 mg, 0.37 mmol), RuphosPd-G3 (16 mg, 0.019 mmol), Ruphos (20 mg, 0.043 mmol) and K₂CO₃ (116 mg, 0.84 mmol) at room temperature. The mixture was stirred at 100° C. for overnight. After cooled to room temperature and LC-MS detection, the solution was filtered through a pad of celite. The filtrate was evaporated in vacuo to give 325 mg crude product which was used directly in the next step. MS (ESI, m/e) [M+H]⁺ 867.8 and 767.9.

### Step 2: 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-6,7-difluorobenzo[d]thiazol-2-amine

To a solution of crude tert-butyl 3-(7-(2-((tert-butoxycarbonyl)amino)-6,7-difluorobenzo[d] thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate (324 mg) in DCM (20 mL) was added TFA (4 mL) at room temperature. The mixture was stirred at room temperature for about 20 h. Then, the mixture was evaporated in vacuo. The residue was diluted with MeOH and 7 M NH₃ in MeOH was added to adjust PH >7. Next, the solvent was removed in vacuo and the residue was purified by pre-HPLC to give the title product (39.57 mg, FA salt). MS (ESI, m/e) [M+H]⁺ 667.8. ¹H NMR (500 MHz, CD₃OD) δ 8.47 (s, 1H), 8.16 (s, 1H), 7.17 (dd, J=10.8, 7.9 Hz, 1H), 5.61-5.31 (m, 1H), 4.77-4.60 (m, 2H), 4.53 (q, J=11.9 Hz, 2H), 4.05 (s, 2H), 3.88 (td, J=13.2, 5.6 Hz, 2H), 3.81-3.73 (m, 3H), 3.63 (dd, J=29.5, 11.6 Hz, 3H), 3.30-3.26 (m, 1H), 2.49 (m, 2H), 2.37-2.29 (m, 1H), 2.22 (dd, J=6.6, 4.6 Hz, 2H), 2.03 (m, 5H).

### Example 86: 7-fluoro-4-(8-fluoro-4-((1S,4R)-2-((2-fluoropropyl)amino)-7-azabicyclo[2.2.1]heptan-7-yl)-2-(((2R,7aS)-2-fluorotetrahydro-1H-pyrrolizin-7a(5H)-yl)methoxy)-6-(trifluoromethyl)quinazolin-7-yl)benzo[d]thiazol-2-amine

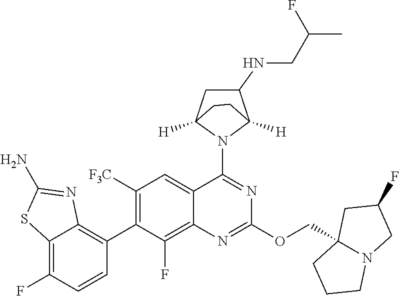 [SMILES: CC(F)CNC1C[C@H]2CC[C@@H]1N2c1nc(OC[C@@]23CCCN2C[C@H](F)C3)nc2c(F)c(-c3ccc(F)c4sc(N)nc34)c(C(F)(F)F)cc12]

Synthetic Route

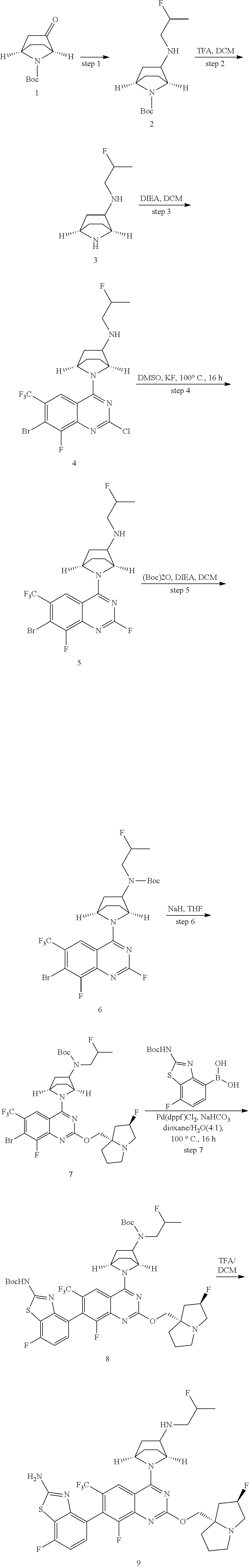

### Step 1 tert-butyl (1S,4R)-2-((2-fluoropropyl)amino)-7-azabicyclo[2.2.1]heptane-7-carboxylate

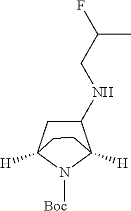 [SMILES: CC(F)CNC1C[C@H]2CC[C@@H]1N2C(=O)OC(C)(C)C]

The reaction mixture of tert-butyl (1S,4R)-2-oxo-7-azabicyclo[2.2.1]heptane-7-carboxylate (650 mg, 2.84 mmol ), 2-fluoropropan-1-amine (656 mg, 8.52 mmol) in MeOH (5 mL) was stirred for 3 hrs at rt. Sodium triacetoxyborohydride (1.2 g, 5.68 mmol) was added and the reaction mixture was stirred for 16 hrs at rt. The reaction solution was concentrated and used in next step directly without further purification. MS (ESI, m/e) [M+1]⁺273.

### Step 2: (1S,4R)—N-(2-fluoropropyl)-7-azabicyclo[2.2.1]heptan-2-amine

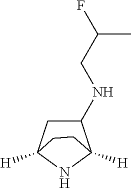

To a solution of tert-butyl (1S,4R)-2-((2-fluoropropyl)amino)-7-azabicyclo[2.2.1]heptane-7-carboxylate (crude) in DCM (16 mL) was added TFA (4 mL) and the solution was stirred for 2 hrs at rt. The reaction solution was concentrated and used in next step directly without further purification. MS (ESI, m/e) [M+1]⁺173.

### Step 3: (1S,4R)-7-(7-bromo-2-chloro-8-fluoro-6-(trifluoromethyl)quinazolin-4-yl)-N-(2-fluoropropyl)-7-azabicyclo[2.2.1]heptan-2-amine

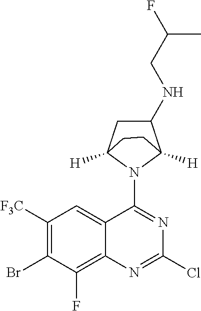 [SMILES: CC(F)CNC1C[C@H]2CC[C@@H]1N2c1nc(Cl)nc2c(F)c(Br)c(C(F)(F)F)cc12]

To the solution of tert-butyl (1S,4R)—N-(2-fluoropropyl)-7-azabicyclo[2.2.1]heptan-2-amine (crude) and 7-bromo-2,4-dichloro-8-fluoro-6-(trifluoromethyl)quinazoline (0.8 g, 2.2 mmol) in DCM (20 mL) was added DIPEA (1.4 g, 4.4 mmol). The mixture was stirred for 2 hrs at rt. The reaction solution was washed with water and dried with Na₂SO₄. The crude product was purified by silica gel column with PE:EA (1:1) to afford the title compound. MS (ESI, m/e) [M+1]⁺499.

### Step 4: (1S,4R)-7-(7-bromo-2,8-difluoro-6-(trifluoromethyl)quinazolin-4-yl)-N-(2-fluoropropyl)-7-azabicyclo[2.2.1]heptan-2-amine

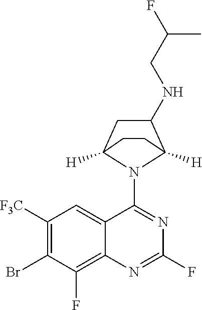 [SMILES: CC(F)CNC1C[C@H]2CC[C@@H]1N2c1nc(F)nc2c(F)c(Br)c(C(F)(F)F)cc12]

To the solution of (1S,4R)-7-(7-bromo-2-chloro-8-fluoro-6-(trifluoromethyl)quinazolin-4-yl)-N-(2-fluoropropyl)-7-azabicyclo[2.2.1]heptan-2-amine (470 mg, 0.94 mmol) in DMSO (20 mL) was added KF (546 mg, 9.4 mmol). The mixture was stirred for 16 hrs at 100° C. The reaction solution was diluted with water and extracted with EtOAc. The organic layer was dried with Na₂SO₄ and purified by silica gel column with PE:EA (1:3) to afford the title product. MS (ESI, m/e) [M+1]⁺483.

### Step 5: tert-butyl ((1S,4R)-7-(7-bromo-2,8-difluoro-6-(trifluoromethyl)quinazolin-4-yl)-7-azabicyclo[2.2.1]heptan-2-yl)(2-fluoropropyl)carbamate

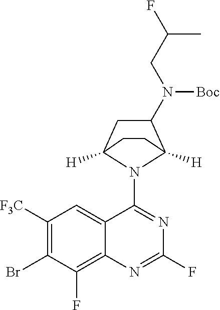 [SMILES: CC(F)CN(C(=O)OC(C)(C)C)C1C[C@H]2CC[C@@H]1N2c1nc(F)nc2c(F)c(Br)c(C(F)(F)F)cc12]

To the solution of (1S,4R)-7-(7-bromo-2,8-difluoro-6-(trifluoromethyl)quinazolin-4-yl)-N-(2-fluoropropyl)-7-azabicyclo[2.2.1]heptan-2-amine (200 mg, 0.41 mmol) in DCE (20 mL) was added DIPEA (270 mg, 2.1 mmol) and Di-tert-butyl dicarbonate (451 mg, 2.1 mmol). The mixture was stirred for 16 hrs at 50° C. The reaction solution was diluted with water and extracted with EtOAc. The organic layer was dried with Na₂SO₄ and purified by silica gel column with PE:EtOAc (1:1) to afford the title product. MS (ESI, m/e) [M+1]⁺583.

### Step 6: tert-butyl ((1S,4R)-7-(7-bromo-8-fluoro-2-(((2R,7aS)-2-fluorotetrahydro-1H-pyrrolizin-7a(5H)-yl)methoxy)-6-(trifluoromethyl)quinazolin-4-yl)-7-azabicyclo[2.2.1]heptan-2-yl)(2-fluoropropyl)carbamate

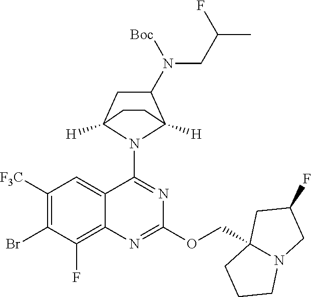 [SMILES: CC(F)CN(C(=O)OC(C)(C)C)C1C[C@H]2CC[C@@H]1N2c1nc(OC[C@@]23CCCN2C[C@H](F)C3)nc2c(F)c(Br)c(C(F)(F)F)cc12]

To the solution of ((2R,7aS)-2-fluorotetrahydro-1H-pyrrolizin-7a(5H)-yl)methanolin (53 mg, 0.33 mmol) in THF (20 mL) was added NaH (13.4 mg, 0.33 mmol, 60% in oil) at rt. The mixture was stirred for 0.5 h at rt, then tert-butyl ((1S,4R)-7-(7-bromo-2,8-difluoro-6-(trifluoromethyl)quinazolin-4-yl)-7-azabicyclo[2.2.1]heptan-2-yl)(2-fluoropropyl)carbamate (130 mg, 0.22 mmol) was added. The mixture was stirred for 16 hrs at rt. The reaction solution was diluted with water and extracted with EtOAc. The organic layer was dried with Na₂SO₄ and purified by Prep-HPLC to afford the title product. MS (ESI, m/e) [M+1]⁺722.

### Step 7: tert-butyl ((1S,4R)-7-(7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-7-azabicyclo[2.2.1]heptan-2-yl)(2-fluoropropyl)carbamate

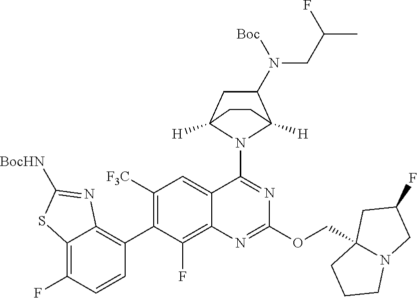 [SMILES: CC(F)CN(C(=O)OC(C)(C)C)C1C[C@H]2CC[C@@H]1N2c1nc(OC[C@@]23CCCN2C[C@H](F)C3)nc2c(F)c(-c3ccc(F)c4sc(NC(=O)OC(C)(C)C)nc34)c(C(F)(F)F)cc12]

To the solution of tert-butyl ((1S,4R)-7-(7-bromo-8-fluoro-2-(((2R,7aS)-2-fluorotetrahydro-1H-pyrrolizin-7a(5H)-yl)methoxy)-6-(trifluoromethyl)quinazolin-4-yl)-7-azabicyclo[2.2.1]heptan-2-yl)(2-fluoropropyl)carbamate (30 mg, 0.04 mmol), (2-((tert-butoxycarbonyl)amino)-7-fluorobenzo[d]thiazol-4-yl)boronic acid (19.5 mg, 0.06 mmol) and K₃PO₄ (26.4 mg, 0.12 mmol) in 1,4-dioxane (20 mL) and water (4 mL) was added Pd(dtbpf)Cl₂ (5.4 mg, 0.008 mmol) at rt. The mixture was stirred for 16 hrs at 100° C. The reaction solution was diluted with water and extracted with EtOAc. The organic layer was dried with Na₂SO₄ and purified by Prep-HPLC (EtOAc) to afford the title product. MS (ESI, m/e) [M+1]⁺910.

### Step 8: 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethoxy)quinazolin-7-yl)-7-fluorobenzo[d]thiazol-2-amine

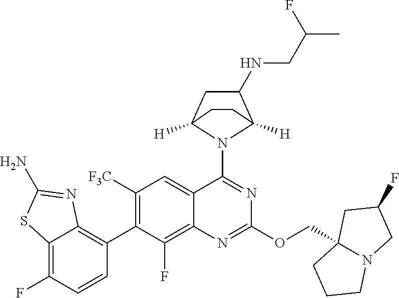 [SMILES: CC(F)CNC1C[C@H]2CC[C@@H]1N2c1nc(OC[C@@]23CCCN2C[C@H](F)C3)nc2c(F)c(-c3ccc(F)c4sc(N)nc34)c(C(F)(F)F)cc12]

To the solution of tert-butyl ((1S,4R)-7-(7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-7-azabicyclo[2.2.1]heptan-2-yl)(2-fluoropropyl)carbamate (30 mg, 0.03 mmol) in DCM (6 mL) was added TFA (2 mL). The mixture was stirred for 2 hrs at rt. The reaction solution was removed and diluted with DCM (5 mL), then two drops of DIPEA was added and the solution was removed. The crude product was purified by Prep-HPLC to afford the title product. MS (ESI, m/e) [M+1]⁺710. ¹H NMR (500 MHz, MeOD) δ 8.54-8.43 (m, 2H), 8.20 (s, 1H), 7.24-7.14 (m, 1H), 7.02-6.91 (m, 1H), 5.41 (d, J=53.5 Hz, 1H), 5.00-4.95 (m, 1H), 4.91-4.88 (m, 1H), 4.79-4.69 (m, 1H), 4.55-4.38 (m, 2H), 3.61-3.40 (m, 4H), 3.27-3.17 (m, 1H), 2.85-2.69 (m, 2H), 2.53-2.33 (m, 3H), 2.30-2.06 (m, 4H), 2.04-1.81 (m, 3H), 1.77-1.69 (m, 1H), 1.38-1.29 (m, 3H), 1.28-1.19 (m, 1H).

### Example 87: 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethoxy)quinazolin-7-yl)-7-fluorobenzo[d]thiazol-2-amine

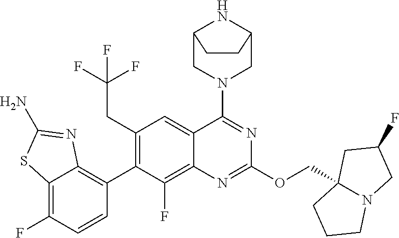 [SMILES: Nc1nc2c(-c3c(CC(F)(F)F)cc4c(N5CC6CCC(C5)N6)nc(OC[C@@]56CCCN5C[C@H](F)C6)nc4c3F)ccc(F)c2s1]

Synthetic Route

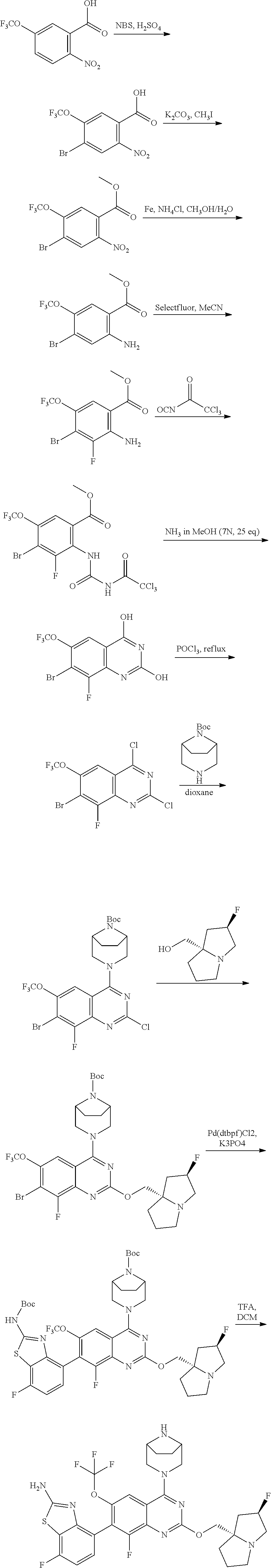

### Step 1: 4-bromo-2-nitro-5-(trifluoromethoxy)benzoic acid

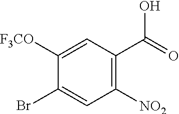 [SMILES: O=C(O)c1cc(OC(F)(F)F)c(Br)cc1[N+](=O)[O-]]

A solution of 2-nitro-5-(trifluoromethoxy)benzoic acid (5 g, 19.91 mmol, 1 equiv) and NBS (3.54 g, 19.91 mmol, 1.0 equiv) in H₂SO₄ (conc., 50 mL) were stirred for 5 hrs at 80° C. The mixture was allowed to cool down to room temperature. The resulting mixture was diluted with ice water (30 mL). The resulting mixture was filtered, and the filter cake was washed with water (3×300 mL). The filtrate was concentrated under reduced pressure to afford the crude title product. MS (ESI, m/e) [M+1]⁺330.0.

### Step 2: methyl 4-bromo-2-nitro-5-(trifluoromethoxy)benzoate

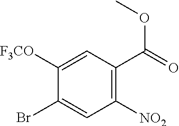 [SMILES: COC(=O)c1cc(OC(F)(F)F)c(Br)cc1[N+](=O)[O-]]

To a stirred solution of 4-bromo-2-nitro-5-(trifluoromethoxy)benzoic acid (3.36 g, 10.18 mmol, 1 equiv) and K₂CO₃ (4.22 g, 30.54 mmol, 3.0 equiv) in DMF was added CH₃I (2.89 g, 20.36 mmol, 2.0 equiv) dropwise at room temperature. The resulting mixture was stirred for 2 hrs at room temperature. The resulting mixture was extracted with EtOAc (3×100 mL). The combined organic layers were washed with brine (3×200 mL), dried over anhydrous Na₂SO₄. After filtration, the filtrate was concentrated under reduced pressure. The residue was purified by silica gel column chromatography, eluted with PE/EtOAc (12:1) to afford the title product.

### Step 3: methyl 2-amino-4-bromo-5-(trifluoromethoxy)benzoate

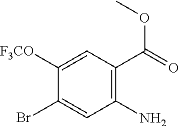 [SMILES: COC(=O)c1cc(OC(F)(F)F)c(Br)cc1N]

To a stirred solution of methyl 4-bromo-2-nitro-5-(trifluoromethoxy)benzoate (1.6 g, 4.65 mmol, 1 equiv) and Fe (1.35 g, 24.18 mmol, 5.2 equiv) in MeOH (20 mL) and water (4 mL) was added NH₄Cl (4.98 g, 93.02 mmol, 20 equiv) in portions at room temperature. The resulting mixture was stirred for 2 hrs at 100° C. under nitrogen atmosphere. The resulting mixture was extracted with EtOAc (3×60 mL). The combined organic layers were washed with brine (3×150 mL), dried over anhydrous Na2SO4. After filtration, the filtrate was concentrated under reduced pressure. The residue was purified by silica gel column chromatography, eluted with PE/EtOAc (10:1) to afford the title product. MS (ESI, m/e) [M+1]⁺314.1.

### Step 4: methyl 2-amino-4-bromo-3-fluoro-5-(trifluoromethoxy)benzoate

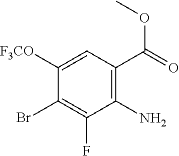 [SMILES: COC(=O)c1cc(OC(F)(F)F)c(Br)c(F)c1N]

A solution of methyl 2-amino-4-bromo-5-(trifluoromethoxy)benzoate (570 mg, 1.81 mmol, 1 equiv) and selectfluor (771.5 mg, 2.17 mmol, 1.2 equiv) in MeCN (10 mL) was stirred overnight at room temperature. The resulting mixture was extracted with EtOAc (3×40 mL). The combined organic layers were washed with brine (3×100 mL), dried over anhydrous Na₂SO₄. After filtration, the filtrate was concentrated under reduced pressure. The residue was purified by Prep-TLC (PE/EtOAc=12:1) to afford the title product. MS (ESI, m/e) [M+1]⁺332.0.

### Step 5: methyl 4-bromo-3-fluoro-2-(3-(2,2,2-trichloroacetyl)ureido)-5-(trifluoromethoxy)benzoate

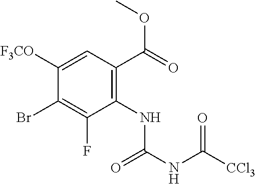

A solution of methyl 2-amino-4-bromo-3-fluoro-5-(trifluoromethoxy)benzoate (120 mg, 0.36 mmol, 1 equiv) and trichloroethanecarbonyl isocyanate (74.8 mg, 0.39 mmol, 1.1 equiv) in THF (2 mL) was stirred for 2 hrs at room temperature. Then, the resulting mixture was concentrated under reduced pressure. The crude product was used in the next step directly without further purification. MS (ESI, m/e) [M+1]⁺200.1.

### Step 6: 7-bromo-8-fluoro-6-(trifluoromethoxy)quinazoline-2,4-diol

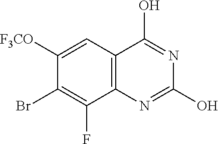 [SMILES: Oc1nc(O)c2cc(OC(F)(F)F)c(Br)c(F)c2n1]

Into a 25 mL round-bottom flask were added methyl 4-bromo-3-fluoro-2-{[(2,2,2-trichloroacetyl)carbamoyl]amino}-5-(trifluoromethoxy)benzoate (90 mg, 0.17 mmol, 1 equiv) and NH3 (g) in MeOH (0.7 mL, 4.45 mmol, 25.7 equiv) at room temperature. The resulting mixture was stirred for 1 h at room temperature. The resulting mixture was extracted with EtOAc (3×20 mL), dried over anhydrous Na2SO4. After filtration, the filtrate was concentrated under reduced pressure. The precipitated solids were collected by filtration and washed with ethyl ether (3×10 mL) to afford the title product. MS (ESI, m/e) [M+1]⁺343.0.

### Step 7: 7-bromo-2,4-dichloro-8-fluoro-6-(trifluoromethoxy)quinazoline

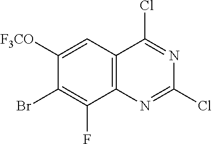 [SMILES: Fc1c(Br)c(OC(F)(F)F)cc2c(Cl)nc(Cl)nc12]

A solution of 7-bromo-8-fluoro-6-(trifluoromethoxy)quinazoline-2,4-diol (150 mg, 0.43 mmol, 1 equiv) and DIEA (0.3 mL) in POCl₃ (3 mL) was stirred for 2 hrs at 110° C. under nitrogen atmosphere. The resulting mixture was concentrated under reduced pressure. The residue was purified by Prep-TLC (PE/EtOAc=5:1) to afford the title product. MS (ESI, m/e) [M+1]⁺379.9.

### Step 8: tert-butyl 3-[7-bromo-2-chloro-8-fluoro-6-(trifluoromethoxy)quinazolin-4-yl]-3,8-diazabicyclo[3.2.1]octane-8-carboxylate

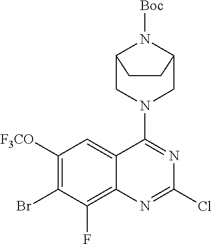 [SMILES: CC(C)(C)OC(=O)N1C2CCC1CN(c1nc(Cl)nc3c(F)c(Br)c(OC(F)(F)F)cc13)C2]

A solution/mixture of 7-bromo-2,4-dichloro-8-fluoro-6-(trifluoromethoxy)quinazoline (55 mg, 0.15 mmol, 1 equiv) and tert-butyl 3,8-diazabicyclo[3.2.1]octane-8-carboxylate (32.27 mg, 0.15 mmol, 1.05 equiv) in 1,4-dioxane (1 mL) was stirred for 1 h at room temperature. The residue was purified by Prep-TLC (PE/EtOAc=5:1) to afford the title product. MS (ESI, m/e) [M+1]⁺ 557.0. ¹H NMR (400 MHz, CDCl₃) δ 7.49 (s, 1H), 4.30 (s, 4H), 3.57 (s, 2H), 1.91-1.88 (m, 2H), 1.63 (s, 2H), 1.45 (s, 9H).

### Step 9: tert-butyl 3-(7-bromo-8-fluoro-2-(((2R,7aS)-2-fluorotetrahydro-1H-pyrrolizin-7a(5H)-yl)methoxy)-6-(trifluoromethoxy)quinazolin-4-yl)-3,8-diazabicyclo[3.2.1]octane-8-carboxylate

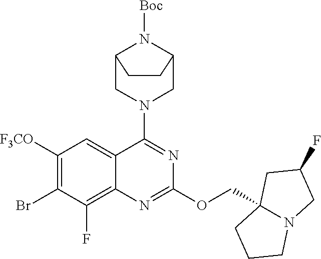 [SMILES: CC(C)(C)OC(=O)N1C2CCC1CN(c1nc(OC[C@@]34CCCN3C[C@H](F)C4)nc3c(F)c(Br)c(OC(F)(F)F)cc13)C2]

The reaction mixture of tert-butyl 3-(7-bromo-2-chloro-8-fluoro-6-(trifluoromethoxy)quinazolin-4-yl)-3,8-diazabicyclo[3.2.1]octane-8-carboxylate (18 mg, 0.032 mmol), ((2R,7aS)-2-fluorotetrahydro-1H-pyrrolizin-7a(5H)-yl)methanol (30.8 mg, 0.192 mmol) and potassium fluoride (18.8 mg, 0.32 mmol) in DMSO (5 mL) was stirred for 16 hrs at 100° C. Water (10 mL) was added to the reaction and the resulting mixture was extracted with EtOAc (30 mL). The organic phase was washed with brine (3×20 mL), dried over anhydrous Na2SO4 and the solvent was removed to afford crude product which was used in next step directly. MS (ESI, m/e) [M+1]⁺678.

### Step 10: tert-butyl 3-(7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethoxy)quinazolin-4-yl)-3,8-diazabicyclo[3.2.1]octane-8-carboxylate

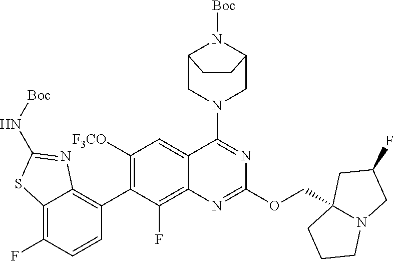 [SMILES: CC(C)(C)OC(=O)Nc1nc2c(-c3c(OC(F)(F)F)cc4c(N5CC6CCC(C5)N6C(=O)OC(C)(C)C)nc(OC[C@@]56CCCN5C[C@H](F)C6)nc4c3F)ccc(F)c2s1]

To a solution of 3 tert-butyl 3-(7-bromo-8-fluoro-2-(((2R,7aS)-2-fluorotetrahydro-1H-pyrrolizin-7a(5H)-yl)methoxy)-6-(trifluoromethoxy)quinazolin-4-yl)-3,8-diazabicyclo[3.2.1]octane-8-carboxylate (14 mg, 0.021 mmol, 1 eq) in 1,4-dioxane (20 mL) and water (4 mL) was added Pd(dtbpf)Cl₂ (2.7 mg, 0.004 mmol), (2-((tert-butoxycarbonyl)amino)-7-fluorobenzo[d]thiazol-4-yl)boronic acid (7.9 mg, 0.025 mmol), and Potassium phosphate tribasic (13.3 mg, 0.063 mmol). The resulting mixture was stirred for 16 hrs at 100° C. Water (30 mL) was added and the resulting mixture was extracted with EtOAc (100 mL). The organic phase was washed with brine (3×50 mL), dried over anhydrous Na₂SO₄ and the solvent was removed to afford crude title product which used in next step directly. MS (ESI, m/e) [M+1]⁺866.

### Step 11: 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethoxy)quinazolin-7-yl)-7-fluorobenzo[d]thiazol-2-amine

To the solution of tert-butyl 3-(7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-(trifluoromethoxy)quinazolin-4-yl)-3,8-diazabicyclo[3.2.1]octane-8-carboxylate (10 mg, 0.012 mmol) in DCM (6 mL) was added TFA (2 mL). The mixture was stirred for 2 hrs at rt. The reaction solution was removed and diluted with DCM (5 mL), then two drops of DIPEA was added and the solution was removed. The crude product was purified with Prep-HPLC to afford the title product. MS (ESI, m/e) [M+1]⁺666. ¹H NMR (500 MHz, CD₃OD) δ 8.57-8.46 (m, 1H), 7.72 (s, 1H), 7.25 (dd, J=8.4, 5.5 Hz, 1H), 6.99 (t, J=8.8 Hz, 1H), 5.50-5.23 (m, 1H), 4.58 (s, 1H), 4.55-4.49 (m, 1H), 4.44-4.36 (m, 2H), 4.34-4.28 (m, 1H), 3.79-3.67 (m, 3H), 3.65-3.58 (m, 1H), 3.48-3.42 (m, 1H), 3.19-3.09 (m, 1H), 2.44-2.27 (m, 2H), 2.24-2.17 (m, 1H), 2.11-2.03 (m, 2H), 1.96-1.89 (m, 3H), 1.87-1.81 (m, 1H), 1.32-1.27 (m, 2H).

### Example 88: 6-(4-(3-oxa-7,9-diazabicyclo[3.3.1]nonan-9-yl)-6-chloro-8-fluoro-2-((2-fluorotetrahydro-1H-pyrrolizin-7a(5H)-yl)methoxy)quinazolin-7-yl)-4-methyl-5-(trifluoromethyl)pyridin-2-amine

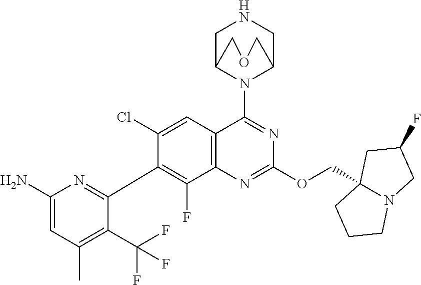 [SMILES: Cc1cc(N)nc(-c2c(Cl)cc3c(N4C5CNCC4COC5)nc(OC[C@@]45CCCN4C[C@H](F)C5)nc3c2F)c1C(F)(F)F]

Synthetic Route

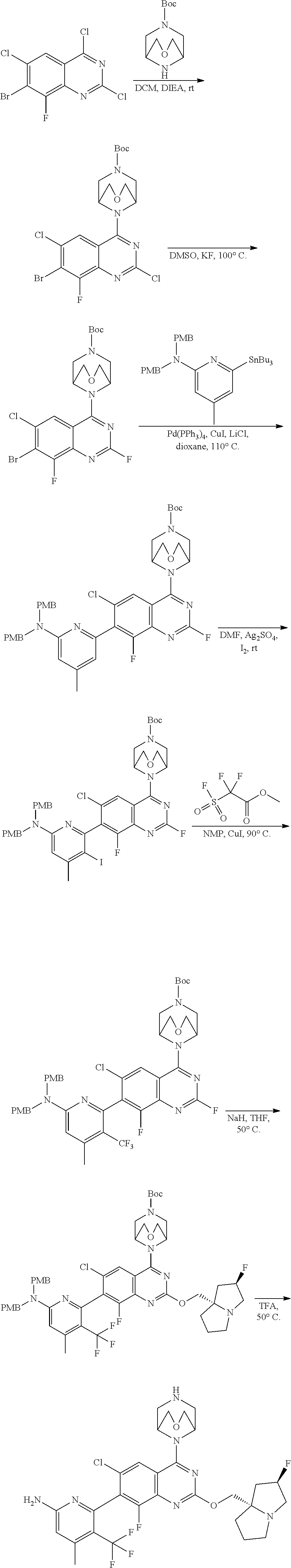

### Step 1: tert-butyl 9-(7-bromo-2,6-dichloro-8-fluoroquinazolin-4-yl)-3-oxa-7,9-diazabicyclo[3.3.1]nonane-7-carboxylate

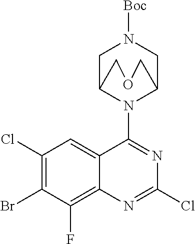 [SMILES: CC(C)(C)OC(=O)N1CC2COCC(C1)N2c1nc(Cl)nc2c(F)c(Br)c(Cl)cc12]

A mixture of 7-bromo-2,4,8-trichloro-6-iodoquinazoline (850 mg, 2.6 mmol), tert-butyl 3-oxa-7,9-diazabicyclo[33.1]nonane-7-carboxylate (590 g, 2.6 mmoL) in DCM (50 mL) and DIPEA (1 mL) was stirred for 2 hours at room temperature. After completion, solvents were directly concentrated and the crude product was purified by silica column (eluting with PE/EtOAc=3/2) to afford the title product (1 g). MS (ESI, m/e) [M+1]⁺ 520.90.

### Step 2: tert-butyl 9-(7-bromo-6-chloro-2,8-difluoroquinazolin-4-yl)-3-oxa-7,9-diazabicyclo[3.3.1]nonane-7-carboxylate

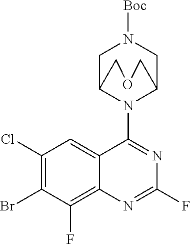 [SMILES: CC(C)(C)OC(=O)N1CC2COCC(C1)N2c1nc(F)nc2c(F)c(Br)c(Cl)cc12]

To a mixture of tert-butyl 9-(7-bromo-2,6-dichloro-8-fluoroquinazolin-4-yl)-3-oxa-7,9-diazabicyclo[33.1-]nonane-7-carboxylate (1 g, 2.5 mmol) in DMSO (30 mL) was added KF (1 g) and the mixture was stirred for 6 hours at 100 VC. After completion, the resulting mixture was poured into water and extracted with EtOAc. The organic layer was combined and concentrated. Then the residue was further purified by silica column (eluting with PE/EA=1/1) to give the title product (900 mg). MS (ESI, m/e) [M+1]⁺505.2.

### Step 3: tert-butyl 9-(7-(6-(bis(4-methoxybenzyl)amino)-4-methylpyridin-2-yl)-6-chloro-2,8-difluoroquinazolin-4-yl)-3-oxa-7,9-diazabicyclo[3.3.1]nonane-7-carboxylate

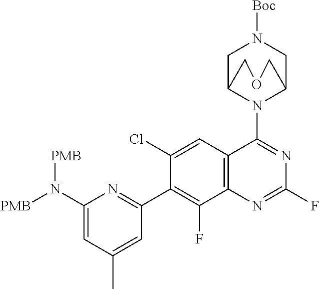

A mixture of N,N-bis(4-methoxybenzyl)-4-methyl-6-(tributylstannyl)pyridin-2-amine (1 g, 1.6 mmol), tert-butyl 9-(7-bromo-6-chloro-2,8-difluoroquinazolin-4-yl)-3-oxa-7,9-diazabicyclo[3.3.1]nonane-7-carboxylate (500 mg, 1 mmol), Pd(PPh₃)₄ (116 mg, 0.1 mmol), CuI (80 mg), LiCl (130 mg) in dioxane (20 mL) was stirred for 4 hours at 110° C. After completion, the resulting mixture was directly concentrated. Then, the crude product was further purified by silica column (eluting with PE/EtOAc=3/2) to give the title product (360 mg). MS (ESI, m/e) [M+1]⁺773.2.

### Step 4: tert-butyl 9-(7-(6-(bis(4-methoxybenzyl)amino)-3-iodo-4-methylpyridin-2-yl)-6-chloro-2,8-difluoroquinazolin-4-yl)-3-oxa-7,9-diazabicyclo[3.3.1]nonane-7-carboxylate

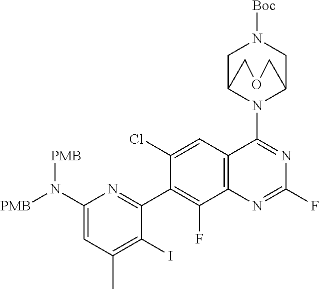

A mixture of tert-butyl 9-(7-(6-(bis(4-methoxybenzyl)amino)-4-methylpyridin-2-yl)-6-chloro-2,8-difluoroquinazolin-4-yl)-3-oxa-7,9-diazabicyclo[3.3.1]nonane-7-carboxylate (360 mg, 0.5 mmol), Ag₂SO₄ (80 mg, 0.25 mmol), I₂ (500 mg, 2 mmol) in DMF (10 mL) was stirred for 3 hour at room temperature. After completion, the resulting mixture was directly poured into water and extracted with EtOAc. The organic layer was concentrated. Then the crude product was further purified by silica column to give the title product (100 mg). MS (ESI, m/e) [M+1]⁺ 899.9.

### Step 5: tert-butyl 9-(7-(6-(bis(4-methoxybenzyl)amino)-3-iodo-4-methylpyridin-2-yl)-6-chloro-2,8-difluoroquinazolin-4-yl)-3-oxa-7,9-diazabicyclo[3.3.1]nonane-7-carboxylate

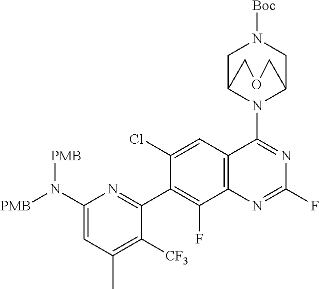

A mixture of tert-butyl 9-(7-(6-(bis(4-methoxybenzyl)amino)-3-iodo-4-methylpyridin-2-yl)-6-chloro-2,8-difluoroquinazolin-4-yl)-3-oxa-7,9-diazabicyclo[3.3.1]nonane-7-carboxylate (100 mg, 0.1 mmol), CuI (200 mg), methyl 2,2-difluoro-2-(fluorosulfonyl)acetate (1 mL) in NMP (8 mL) was stirred for 4 hours at 90° C. After completion, the resulting mixture was directly poured into water and extracted with EtOAc. The organic layer concentrated and further purified by silica column to give the title product (48 mg). MS (ESI, m/e) [M+1]⁺841.2.

### Step 6: tert-butyl 9-(7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3-oxa-7,9-diazabicyclo[3.3.1]nonane-7-carboxylate

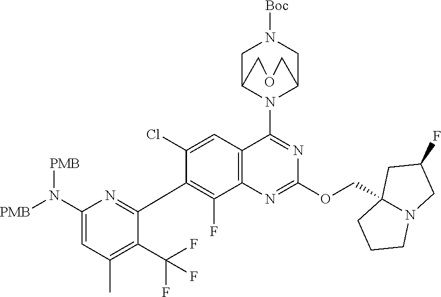

To a solution of ((2S,4R)-4-fluoro-1-methylpyrrolidin-2-yl)methanol (8 mg) in THF (8 mL) was added NaH (20 mg) and the mixture was stirred for 30 mins at room temperature. Then, tert-butyl 9-(7-(6-(bis(4-methoxybenzyl)amino)-3-iodo-4-methylpyridin-2-yl)-6-chloro-2,8-difluoroquinazolin-4-yl)-3-oxa-7,9-diazabicyclo[3.3.1]nonane-7-carboxylate (48 mg, 0.6 mmol) was added. The reaction mixture was stirred for 2 hours at room temperature. After completion, the reaction was quenched by H₂O. Solvents were evaporated and the crude product was purified by silica column (eluting with PE/EtOAc=1/9) to give the title product (50 mg). MS (ESI, m/e) [M+1]⁺980.3.

### Step 7: 6-(4-(3-oxa-7,9-diazabicyclo[3.3.1]nonan-9-yl)-6-chloro-8-fluoro-2-((2-fluorotetrahydro-1H-pyrrolizin-7a(5H)-yl)methoxy)quinazolin-7-yl)-4-methyl-5-(trifluoromethyl)pyridin-2-amine

A mixture of tert-butyl 9-(7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3-oxa-7,9-diazabicyclo[3.3.1]nonane-7-carboxylate (50 mg, 0.05 mmol) in TFA (6 mL) was stirred for 4 hours at 55° C. After completion, the resulting mixture was directly concentrated and the residue was adjusted to pH=9 with DIPEA. Then, the residues were purified by C18 column (eluting with H₂O/CH₃CN=3/1) to afford the title product (9.9 mg, FA salt). ¹H NMR (500 MHz, CD₃OD) δ 8.47 (s, 1H), 7.77 (s, 1H), 6.61 (s, 1H), 5.41-5.51 (m, 1H), 4.70-4.76 (m, 2H), 4.43-4.56 (m, 2H), 4.20-4.31 (m, 4H), 3.56-3.76 (m, 7H), 3.25-3.30 (m, 1H), 2.03-2.51 (m, 9H). MS (ESI, m/e) [M+1]⁺640.0.

### Example 89: 4-(4-(3,8-diazabicyclo[3.2.1]octan-3-yl)-6-chloro-8-fluoro-2-(((4R)-4-fluoro-1,2-dimethylpyrrolidin-2-yl)methoxy)quinazolin-7-yl)-7-fluorobenzo[d]thiazol-2-amine

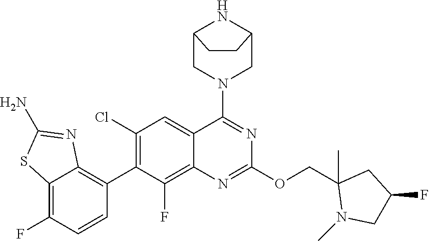 [SMILES: CN1C[C@H](F)CC1(C)COc1nc(N2CC3CCC(C2)N3)c2cc(Cl)c(-c3ccc(F)c4sc(N)nc34)c(F)c2n1]

Synthetic Route

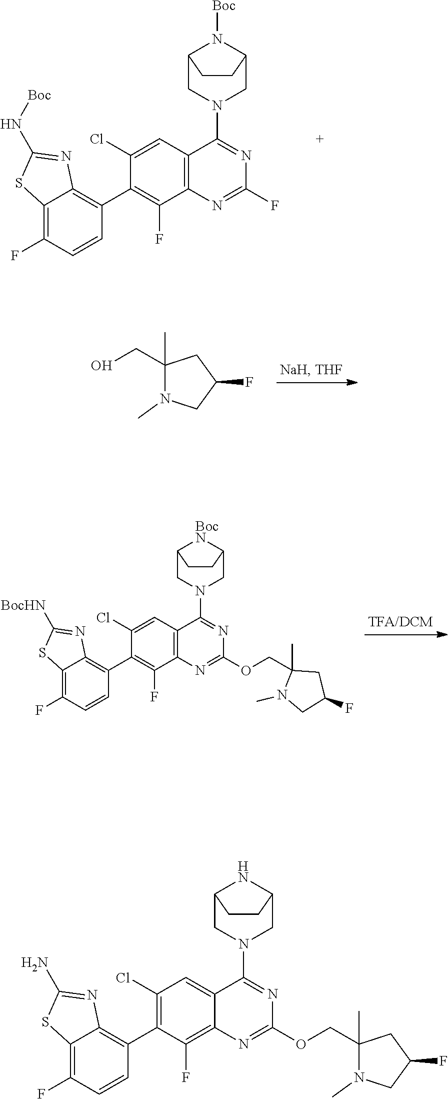

### Step 1: tert-butyl 3-(7-(2-((tert-butoxycarbonyl)amino)-7-fluorobenzo[d]thiazol-4-yl)-6-chloro-8-fluoro-2-(((4R)-4-fluoro-1,2-dimethylpyrrolidin-2-yl)methoxy)quinazolin-4-yl)-3,8-diazabicyclo[3.2.1]octane-8-carboxylate

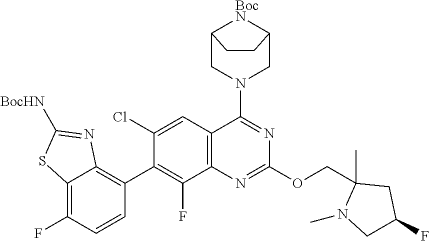 [SMILES: CN1C[C@H](F)CC1(C)COc1nc(N2CC3CCC(C2)N3C(=O)OC(C)(C)C)c2cc(Cl)c(-c3ccc(F)c4sc(NC(=O)OC(C)(C)C)nc34)c(F)c2n1]

To a solution of ((4R)-4-fluoro-1,2-dimethylpyrrolidin-2-yl)methanol (20 mg, 0.134 mmol) in THF (3 mL) was added NaH (11 mg, 0.267 mmol), and the mixture was stirred at room temperature for 0.5 hour, then tert-butyl 3-(7-(2-((tert-butoxycarbonyl)amino)-7-fluorobenzo[d]thiazol-4-yl)-6-chloro-2,8-difluoroquinazolin-4-yl)-3,8-diazabicyclo[3.2.1]octane-8-carboxylate (60 mg, 0.089 mmol) in THF (1.0 mL) was added, and then the mixture was stirred for 4 hours at room temperature. The reaction mixture was diluted with EtOAc and water, extracted with EtOAc twice, and the organic layer was combined, dried over sodium sulfate and evaporated. The residue was purified by chromatography column on silica (eluting with MeOH/DCM=5-10%) to give the title product. MS (ESI, m/e) [M+1]⁺804.

### Step 2: 4-(4-(3,8-diazabicyclo[3.2.1]octan-3-yl)-6-chloro-8-fluoro-2-(((4R)-4-fluoro-1,2-dimethylpyrrolidin-2-yl)methoxy)quinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-6-chloro-8-fluoro-2-(((4R)-4-fluoro-1,2-dimethylpyrrolidin-2-yl)methoxy)quinazolin-4-yl)-3,8-diazabicyclo[3.2.1]octane-8-carboxylate (40 mg, 0.05 mmol) in DCM (6 mL) was added TFA (1.5 mL) and the mixture was stirred for 4 hours at room temperature. Then, the resulting mixture was evaporated at room temperature to give the crude product. This crude product was mixed with DCM (20 mL)/H₂O (10 mL), and the pH was adjusted to 9. The mixture was extracted with DCM/MeOH (v=10/1) twice, and the organic layer was combined and concentrated. The residue was purified by chromatography column on silica (eluting with MeOH/DCM=20%) to give the title product. ¹H NMR (500 MHz, CD₃OD) δ 7.86 (s, 1H), 7.21 (dd, J=8.3, 5.4 Hz, 1H), 6.98 (t, J=8.8 Hz, 1H), 5.16 (dt, J=55.1, 5.2 Hz, 1H), 4.59-4.33 (m, 4H), 3.77-3.55 (m, 4H), 3.23-3.11 (m, 1H), 3.04-2.84 (m, 1H), 2.43 (s, 3H), 2.36-2.05 (m, 2H), 1.93-1.77 (m, 4H), 1.19 (s, 3H), MS (ESI, m/e) [M+H]⁺ 604.3.

### Example 90: 4-(4-(3,9-diazabicyclo[3.3.1]nonan-3-yl)-8-fluoro-2-(((2R,7aS)-2-fluorotetrahydro-1H-pyrrolizin-7a(5H)-yl)methoxy)-6-(trifluoromethyl) quinazolin-7-yl)-7-fluorobenzo[d]thiazol-2-amine

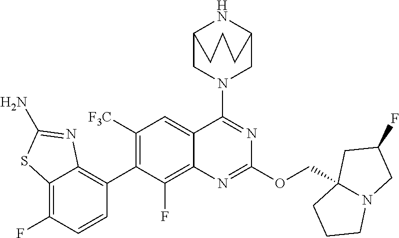 [SMILES: Nc1nc2c(-c3c(C(F)(F)F)cc4c(N5CC6CCCC(C5)N6)nc(OC[C@@]56CCCN5C[C@H](F)C6)nc4c3F)ccc(F)c2s1]

Synthetic Route

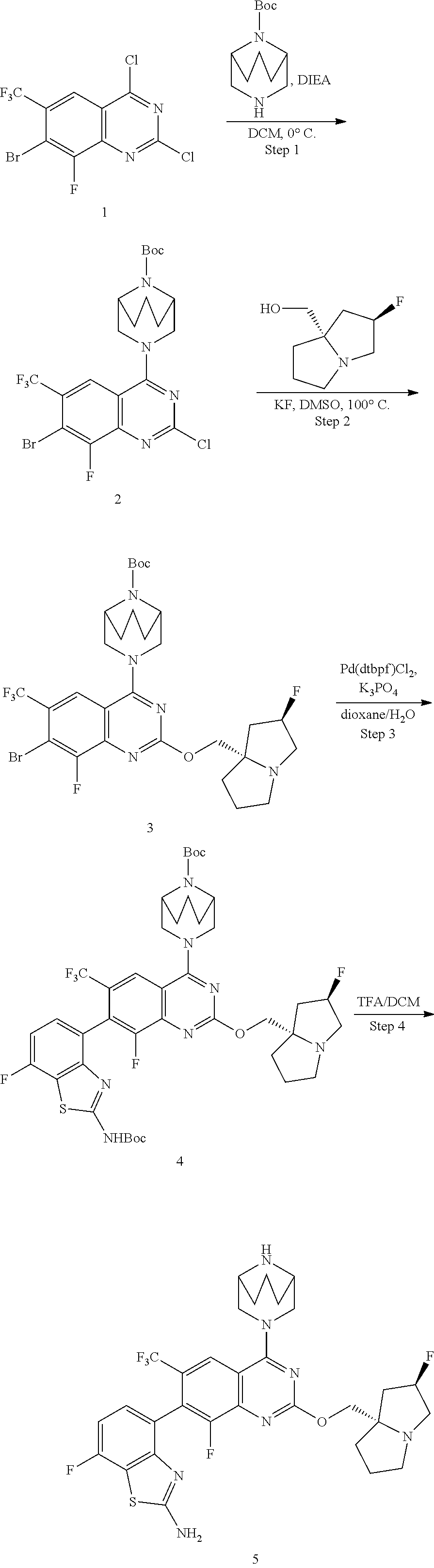

### Step 1: tert-butyl 3-(7-bromo-2-chloro-8-fluoro-6-(trifluoromethyl) quinazolin-4-yl)-3,9-diazabicyclo[3.3.1]nonane-9-carboxylate

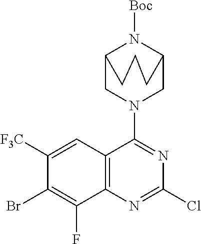 [SMILES: CC(C)(C)OC(=O)N1C2CCCC1CN(c1nc(Cl)nc3c(F)c(Br)c(C(F)(F)F)cc13)C2]

To a solution of 7-bromo-2,4-dichloro-8-fluoro-6-(trifluoromethyl) quinazoline (400 mg, 1.1 mmol) in DCM (10 mL) was added DIEA (427 mg, 3.3 mmol), then to the above mixture was added the solution of tert-butyl 3,9-diazabicyclo[3.3.1] nonane-9-carboxylateoxyborohydride (274 mg, 1.2 mmol) in DCM dropwise at 0° C. . The reaction mixture was stirred for 1 h at 0° C. . The resulting mixture was concentrated and purified by flash column chromatography (eluting with PE/EA=5/1) to give the desired product (470 mg, 77% ). MS (ESI, m/e) [M+1]⁺554.8.

### Step 2: tert-butyl 3-(7-bromo-8-fluoro-2-(((2R,7aS)-2-fluorotetrahydro-1H-pyrrolizin-7a(5H)-yl)methoxy)-6-(trifluoromethyl)quinazolin-4-yl)-3,9-diazabicyclo[13.3.1] nonane-9-carboxylate

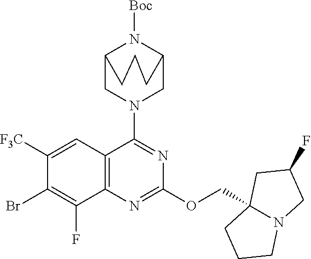 [SMILES: CC(C)(C)OC(=O)N1C2CCCC1CN(c1nc(OC[C@@]34CCCN3C[C@H](F)C4)nc3c(F)c(Br)c(C(F)(F)F)cc13)C2]

To a solution of tert-butyl 3-(7-bromo-2-chloro-8-fluoro-6-(trifluoro methyl)quinazolin-4-yl)-3,9-diazabicyclo[3.3.1]nonane-9-carboxylate (300 mg, 0.54 mmol) in DMSO (5 mL) was added KF (189 mg, 3.24 mmol) and ((2R,7aS)-2-fluoro tetrahydro-1H-pyrrolizin-7a(5H)-yl)methanol (258 mg, 1.62 mmol), the resulting mixture was stirred for overnight at 100° C. The resulting cooled mixture was extracted with EA, combined organic phases and concentrated under reduced pressure. The crude was purified by flash column chromatography on silica (eluting with DCM/MeOH=20/1) to give the title product (295 mg, 80%). MS (ESI, m/e) [M+1]⁺676.4.

### Step 3: tert-butyl 3-(7-(2-((tert-butoxycarbonyl)amino)-7-fluorobenzo[d] thiazol-4-yl)-8-fluoro-2-(((2R,7aS)-2-fluorotetrahydro-1H-pyrrolizin-7a(5H-yl)methoxy)-6-(trifluoromethyl)quinazolin-4-yl)-3,9-diazabicyclo[n3.3.1]nonane-9-carboxylate

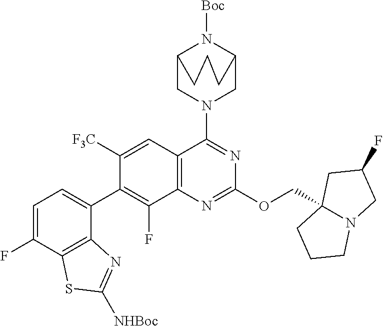 [SMILES: CC(C)(C)OC(=O)Nc1nc2c(-c3c(C(F)(F)F)cc4c(N5CC6CCCC(C5)N6C(=O)OC(C)(C)C)nc(OC[C@@]56CCCN5C[C@H](F)C6)nc4c3F)ccc(F)c2s1]

To a mixture of tert-butyl 3-(7-bromo-8-fluoro-2-(((2R,7aS)-2-fluoro tetrahydro-1H-pyrrolizin-7a(5H)-yl)methoxy)-6-(trifluoromethyl)quinazolin-4-yl)-3,9-diazabicyclo[3.3.1]nonane-9-carboxylate (290 mg, 0.43 mmol), (2-((tert-butoxycarbonyl) amino)-7-fluorobenzo[d]thiazol-4-yl)boronic acid (200 mg, 0.64 mmol), K₃PO₄ (270 mg, 3.0 mmol), Pd(dtbpf)Cl₂ (60 mg, 0.09 mmol) was added dioxane (5.0 mL) and H₂O (1.0 mL), and stirred at 100° C. for 2 hours. The resulting cooled mixture was concentrated and purified by flash column chromatography (DCM/MeOH=20/1) to give the title product (280 mg, 75%). MS (ESI, m/e) [M+1]⁺864.5.

### Step 4: 4-(4-(3,9-diazabicyclo[3.3.1]nonan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

To a solution of tert-butyl 3-(7-(2-((tert-butoxycarbonyl)amino)-7-fluoro benzo[d]thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3,9-diazabicyclo[3.3.1]nonane-9-carboxylate (100 mg, 0.12 mmol) in DCM (6.0 mL) was added TFA (3.0 mL), the resulting mixture was stirred for 2 hours at room temperature. The resulting solution was washed with NaHCO₃/H₂O (Sat. 20 mL) and concentrated to give a residue which was further purified by Prep-HPLC to give the title compound (25.4 mg, 33%). ¹H NMR (500 MHz, CD₃OD) δ 8.22 (s, 1H), 7.20 (dd, J=8.3, 5.4 Hz, 1H), 6.97 (t, J=8.8 Hz, 1H), 5.39 (d, J=53.1 Hz, 1H), 4.74 (dd, J=24.5, 13.7 Hz, 2H), 4.43 (dd, J=32.0, 11.2 Hz, 2H), 3.89 (dd, J=21.2, 13.8 Hz, 2H), 3.51 (d, J=51.6 Hz, 5H), 3.17 (td, J=10.0, 5.8 Hz, 1H), 2.49-2.28 (m, 2H), 2.27-2.19 (m, 1H), 2.09 (m, 8H), 1.66 (d, J=6.5 Hz, 1H). MS (ESI, m/e) [M+H]⁺ 663.90.

### Example 91: 4-(4-(3,8-diazabicyclo[3.2.1]octan-3-yl)-6-(2,2-difluorocyclopropyl)-8-fluoro-2-((2-fluorotetrahydro-1H-pyrrolizin-7a(5H)-yl)methoxy)quinazolin-7-yl)-7-fluorobenzo[d]thiazol-2-amine

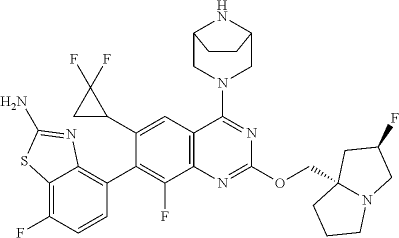 [SMILES: Nc1nc2c(-c3c(C4CC4(F)F)cc4c(N5CC6CCC(C5)N6)nc(OC[C@@]56CCCN5C[C@H](F)C6)nc4c3F)ccc(F)c2s1]

Synthetic Route

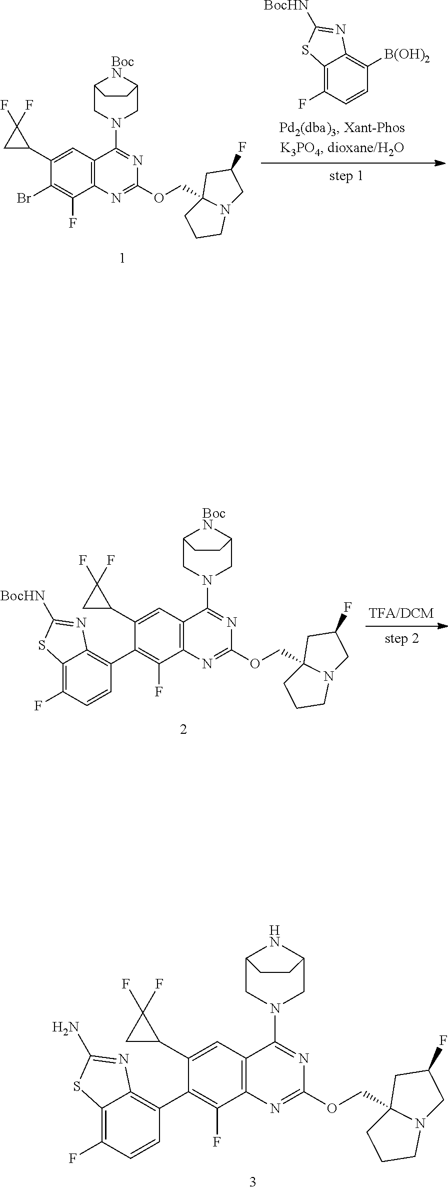 [SMILES: CC(C)(C)OC(=O)N1C2CCC1CN(c1nc(OC[C@@]34CCCN3C[C@H](F)C4)nc3c(F)c(Br)c(C4CC4(F)F)cc13)C2 ; CC(C)(C)OC(=O)Nc1nc2c(B(O)O)ccc(F)c2s1 ; CC(C)(C)OC(=O)Nc1nc2c(-c3c(C4CC4(F)F)cc4c(N5CC6CCC(C5)N6C(=O)OC(C)(C)C)nc(OC[C@@]56CCCN5C[C@H](F)C6)nc4c3F)ccc(F)c2s1 ; Nc1nc2c(-c3c(C4CC4(F)F)cc4c(N5CC6CCC(C5)N6)nc(OC[C@@]56CCCN5C[C@H](F)C6)nc4c3F)ccc(F)c2s1]

### Step 1: tert-butyl 3-(7-(2-((tert-butoxycarbonyl)amino)-7-fluoro benzo[d]thiazol-4-yl)-6-(2,2-difluorocyclopropyl)-8-fluoro-2-((2-fluorotetrahydro-1H-pyrrolizin-7a(5H)-yl)methoxy)quinazolin-4-yl)-3,8-diazabicyclo[3.2.1]octane-8carboxylate

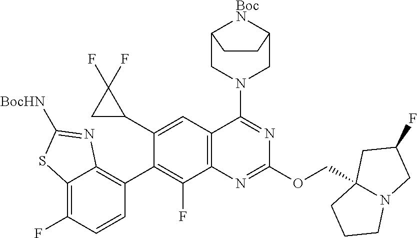 [SMILES: CC(C)(C)OC(=O)Nc1nc2c(-c3c(C4CC4(F)F)cc4c(N5CC6CCC(C5)N6C(=O)OC(C)(C)C)nc(OC[C@@]56CCCN5C[C@H](F)C6)nc4c3F)ccc(F)c2s1]

To a solution of tert-butyl 3-(7-bromo-6-(2,2-difluorocyclopropyl)-8-fluoro-2-((2-fluorotetrahydro-1H-pyrrolizin-7a(5H)-yl)methoxy)quinazolin-4-yl)-3,8-diazabicyclo[3.2.1]octane-8-carboxylate (70 mg, 0.10 mmol) and (2-((tert-butoxy carbonyl)amino)-7-fluorobenzo[d]thiazol-4-yl)boronic acid (70 mg, 0.22 mmol) in 1,4-dioxane/water (2.5 mL/0.5 mL ) was added tris(dibenzylideneacetone)dipalladium (28 mg, 0.03 mmol), 9,9-dimethyl-4,5-bis(diphenylphosphino)xanthene (28 mg, 0.05 mmol) and tripotassium orthophosphate (105 mg, 0.49 mmol). The reaction was stirred at reflux for 2 h, then it was cooled to room temperature. The mixture was diluted with dichloromethane and water and the organic layer was combined, dried over sodium sulfate and evaporated. The residue was purified by chromatography on silica to give the title compound (80 mg, 90%). MS (ESI, m/e) [M+H]⁺ 858.2.

### Step 2: 4-(4-(3,8-diazabicyclo[3.2.1]octan-3-yl)-6-(2,2-difluoro cyclopropyl)-8-fluoro-2-((2-fluorotetrahydro-1H-pyrrolizin-7a(5H)-yl)methoxy)quinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-6-(2,2-difluorocyclopropyl)-8-fluoro-2-((2-fluorotetrahydro-1H-pyrrolizin-7a(5H)-yl)methoxy)quinazolin-4-yl)-3,8-diazabicyclo[3.2.1]octane-8-carboxylate (70 mg, 0.08 mmol) in dichloromethane (3 mL) was added trifluoroacetic acid (2 mL) at room temperature. After the addition, the mixture was stirred at room temperature for 2 h. Then the solution was evaporated, and the residue was adjusted to pH=11 with aq sodium carbonate. The mixture was mixed with dichloromethane:methanol=10:1, and the organic layer was dried over sodium sulfate and evaporated. The residue was purified by Prep-HPLC to give the title compound (32 mg, 61%). ¹H NMR (500 MHz, CD₃OD) δ 7.62-7.60 (d, 1H, J=9.5 Hz), 7.26-7.20 (m, 1H), 7.02-6.97 (m, 1H), 5.43-5.32 (m, 1H), 4.58-4.30 (m, 4H), 3.79-3.59 (m, 4H), 3.55-3.33 (m, 3H), 3.19-3.11 (m, 1H), 2.70-2.59 (m, 1H), 2.48-2.18 (m, 3H), 2.13-2.06 (m, 2H), 2.00-1.80 (m, 5H), 1.72-1.39 (m, 2H). MS (ESI, m/e) [M+H]⁺ 657.9.

### Example 92: 6-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-((trifluoromethyl)thio) quinazolin-7-yl)-4-methyl-5-(trifluoromethyl)pyridin-2-amine

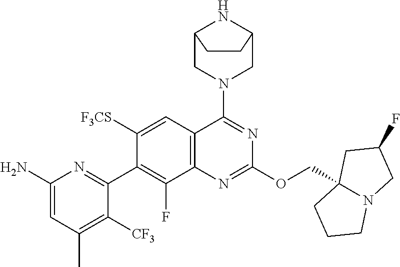

Synthetic Route

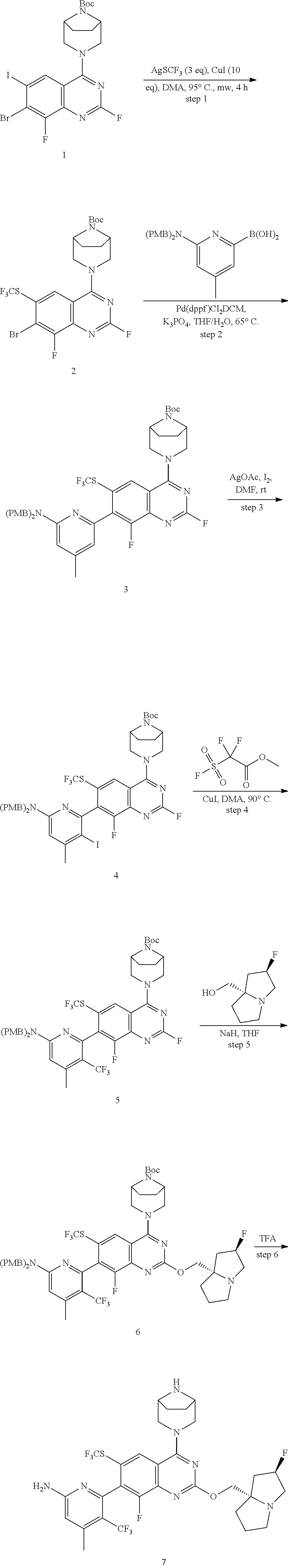

### Step 1: tert-butyl 3-(7-bromo-2,8-difluoro-6-((trifluoromethyl)thio) quinazolin-4-yl)-3,8-diazabicyclo[3.2.1]octane-8-carboxylate

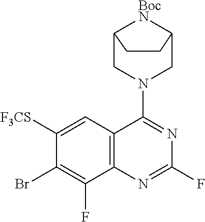

A mixture of tert-butyl 3-(7-bromo-2,8-difluoro-6-iodoquinazolin-4-yl)-3,8-diazabicyclo[3.2.1]octane-8-carboxylate (200 mg, 0.34 mmol), [(trifluoromethyl) sulfanyl]silver (215 mg, 1.03 mmol) and CuI (655 mg, 3.44 mmol) in DMA (13 mL) was stirred for 4 h at 95° C. under microwave. The resulting mixture was filtered. The resulting mixture was extracted with EtOAc. The combined organic layers were washed with brine, dried over anhydrous Na₂SO₄. After filtration, the filtrate was concentrated under reduced pressure. The residue was purified by Prep-TLC (PE/EA=3:1) to give the title product (268 mg, 80% yield). MS (ESI, m/e) [M+H]⁺ 555.05.

### Step 2: tert-butyl 3-(7-(6-(bis(4-methoxybenzyl)amino)-4-methyl pyridin-2-yl)-2,8-difluoro-6-((trifluoromethyl)thio)quinazolin-4-yl)-3,8-diazabicyclo [3.2.1]octane-8-carboxylate

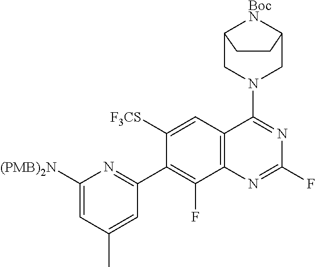

To a stirred mixture of tert-butyl 3-(7-bromo-2,8-difluoro-6-((trifluoro methyl)sulfanyl)quinazolin-4-yl)-3,8-diazabicyclo[3.2.1]octane-8-carboxylate (248 mg, 0.44 mmol) and 6-(bis((4-methoxyphenyl)methyl)amino)-4-methylpyridin-2-ylboronic acid (525 mg, 1.34 mmol) in THF (6.5 mL) and H₂O (1.3 mL) was added K₃PO₄ (189 mg, 0.89 mmol) and Pd(dppf)Cl₂—CH₂Cl₂ (36 mg, 0.04 mmol) in portions at rt. The resulting mixture was stirred for 2 h at 65° C. under nitrogen atmosphere. The resulting mixture was extracted with EtOAc. The combined organic layers were washed with brine, dried over anhydrous Na₂SO₄. After filtration, the filtrate was concentrated under reduced pressure. The residue was purified by combi-flash (PE/EA=7:1) to give the title product (279 mg, 75% yield). MS (ESI, m/e) [M+H]⁺ 823.30.

### Step 3: tert-butyl 3-(7-(6-(bis(4-methoxybenzyl)amino)-3-iodo-4-methyl pyridin-2-yl)-2,8-difluoro-6-((trifluoromethyl)thio)quinazolin-4-yl)-3,8-diazabicyclo [3.2.1]octane-8-carboxylate

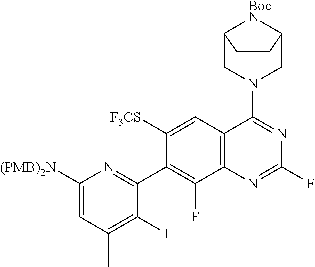

To a stirred mixture of tert-butyl 3-(7-(6-(bis((4-methoxyphenyl)methyl) amino)-4-methylpyridin-2-yl)-2,8-difluoro-6-((trifluoromethyl)sulfanyl)quinazolin-4-yl)-3,8-diazabicyclo[3.2.1]octane-8-carboxylate (259 mg, 0.31 mmol) and AgOAc (118 mg, 0.70 mmol) in DMF (6.5 mL) was added I₂ (239 mg, 0.94 mmol) in portions at rt. The resulting mixture was stirred for 2 h at rt. The resulting mixture was extracted with EtOAc. The combined organic layers were washed with Na₂S₂O₃ and brine, dried over anhydrous Na₂SO₄. After filtration, the filtrate was concentrated under reduced pressure. The residue was purified by Prep-TLC (PE/EA=3:1) to give the title product (213 mg, 71% yield). MS (ESI, m/e) [M+H]⁺ 949.15.

### Step 4: tert-butyl 3-(7-(6-(bis(4-methoxybenzyl)amino)-4-methyl-3-(tri fluoromethyl)pyridin-2-yl)-2,8-difluoro-6-((trifluoromethyl)thio)quinazolin-4-yl)-3,8-diazabicyclo[3.2.1]octane-8-carboxylate

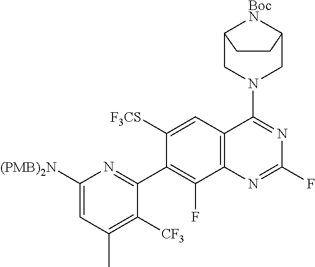

A solution of tert-butyl 3-(7-(6-(bis((4-methoxyphenyl)methyl) amino)-3-iodo-4-methylpyridin-2-yl)-2,8-difluoro-6-((trifluoromethyl)sulfanyl) quinazolin-4-yl)-3,8-diazabicyclo[3.2.1]octane-8-carboxylate (213 mg, 0.22 mmol) and CuI (171 mg, 0.89 mmol) in DMA (8 mL) was stirred for 10 min at rt. To the above mixture was added methyl 2,2-difluoro-2-sulfoacetate (431 mg, 2.24 mmol) dropwise portions. The resulting mixture was stirred for 4 h at 90° C. The resulting mixture was extracted with EtOAc. The combined organic layers were washed with brine, dried over anhydrous Na₂SO₄. After filtration, the filtrate was concentrated under reduced pressure. The residue was purified by Prep-TLC (PE/EA=3:1) to give the title product (177 mg, 88% yield). MS (ESI, m/e) [M+H]⁺ 891.50.

### Step 5: tert-butyl 3-(7-(6-(bis(4-methoxybenzyl)amino)-4-methyl-3-(tri fluoromethyl)pyridin-2-yl)-8-fluoro-2-(((2R,7aS)-2-fluorotetrahydro-1H-pyrrolizin-7a(5H)-yl)methoxy)-6-((trifluoromethyl)thio)quinazolin-4-yl)-3,8-diazabicyclo[3.2.1] octane-8-carboxylate

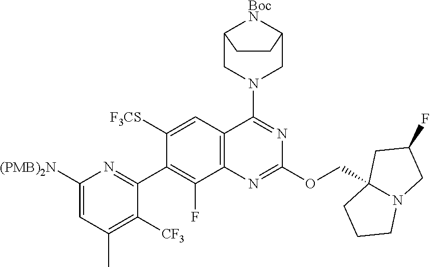

To a solution of ((2R,7aS)-2-fluoro-hexahydropyrrolizin-7a-yl)methanol (34 mg, 0.21 mmol) in THF (4 mL) was added NaH (60% in oil, 11.92 mg, 0.29 mmol) at 0° C. The mixture was stirred for 15 min. tert-butyl 3-(7-(6-(bis((4-methoxyphenyl) methyl)amino)-4-methyl-3-(trifluoromethyl)pyridin-2-yl)-2,8-difluoro-6-((trifluoro methyl)sulfanyl)quinazolin-4-yl)-3,8-diazabicyclo[3.2.1]octane-8-carboxylate (177 mg, 0.19 mmol) was added and the mixture was allowed to warm to rt and stirred for 2 h at rt. The reaction mixture was quenched by ice water. The resulting mixture was extracted with EtOAc. The combined organic layers were washed with brine, dried over anhydrous Na₂SO₄. After filtration, the filtrate was concentrated under reduced pressure. The residue was purified by Prep-TLC (EA=100%) to give the title product (150 mg, 73% yield). MS (ESI, m/e) [M+H]⁺ 1030.75.

### Step 6: 6-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-((trifluoromethyl)thio) quinazolin-7-yl)-4-methyl-5-(trifluoromethyl)pyridin-2-amine

A solution of tert-butyl 3-(7-(6-(bis(4-methoxybenzyl)amino)-4-methyl-3-(trifluoromethyl) pyridin-2-yl)-8-fluoro-2-(((2R,7aS)-2-fluorotetrahydro-1H-pyrrolizin-7a(5H)-yl)methoxy)-6-((trifluoro methyl)thio)quinazolin-4-yl)-3,8-diazabicyclo[3.2.1] octane-8-carboxylate (150 mg, 0.146 mmol, 1 equiv) in trifluoroacetaldehyde (4 mL) was stirred for 2 h at 50° C. The resulting mixture was concentrated under reduced pressure. The residue was purified by PREP-HPLC (Mobile Phase A: Water (10 mmol/L NH₄HCO₃+0.1% NH₃·H₂O), Mobile Phase B: ACN; Flow rate: 60 mL/min; Gradient: 25% B to 55% B in 8 min, 55% B) to give the title product (16.3 mg, 16% yield). ¹H NMR (400 MHz, DMSO-d₆) δ 8.13 (s, 1H), 6.84 (s, 2H), 6.49 (s, 1H), 5.34 (m, 1H), 4.26-4.17 (m, 2H), 4.10 (dd, J=10.3, 7.8 Hz, 1H), 4.00 (dd, J=10.4, 6.9 Hz, 1H), 3.70-3.50 (m, 4H), 3.12-3.05 (m, 2H), 3.02 (d, J=12.2 Hz, 1H), 2.82 (q, J=9.0, 8.6 Hz, 1H), 2.37 (dd, J=2.9, 1.4 Hz, 3H), 2.16-1.98 (m, 3H), 1.79 (dd, J=25.1, 11.7 Hz, 3H), 1.64 (m, 4H). MS (ESI, m/e) [M+H]⁺ 690.10.

### Example 93: 4-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-5,7-difluorobenzo[d] thiazol-2-amine

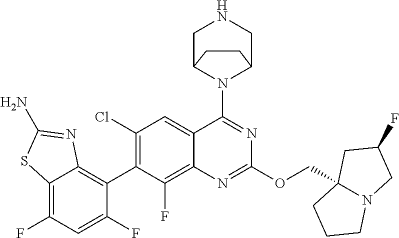 [SMILES: Nc1nc2c(-c3c(Cl)cc4c(N5C6CCC5CNC6)nc(OC[C@@]56CCCN5C[C@H](F)C6)nc4c3F)c(F)cc(F)c2s1]

Synthetic Route

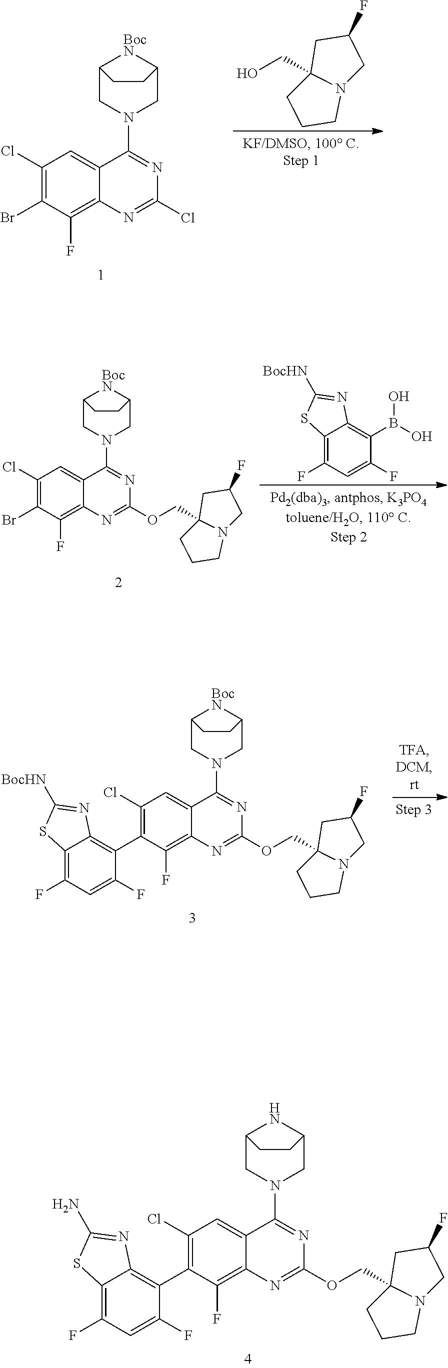 [SMILES: CC(C)(C)OC(=O)N1C2CCC1CN(c1nc(Cl)nc3c(F)c(Br)c(Cl)cc13)C2 ; OC[C@@]12CCCN1C[C@H](F)C2 ; CC(C)(C)OC(=O)N1C2CCC1CN(c1nc(OC[C@@]34CCCN3C[C@H](F)C4)nc3c(F)c(Br)c(Cl)cc13)C2 ; CC(C)(C)OC(=O)Nc1nc2c(B(O)O)c(F)cc(F)c2s1 ; CC(C)(C)OC(=O)Nc1nc2c(-c3c(Cl)cc4c(N5CC6CCC(C5)N6C(=O)OC(C)(C)C)nc(OC[C@@]56CCCN5C[C@H](F)C6)nc4c3F)c(F)cc(F)c2s1 ; Nc1nc2c(-c3c(Cl)cc4c(N5CC6CCC(C5)N6)nc(OC[C@@]56CCCN5C[C@H](F)C6)nc4c3F)c(F)cc(F)c2s1]

### Step 1: tert-butyl 8-(7-bromo-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-3-carboxylate

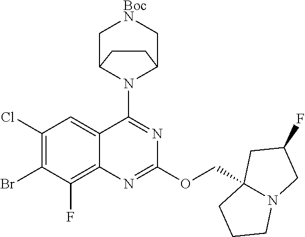 [SMILES: CC(C)(C)OC(=O)N1CC2CCC(C1)N2c1nc(OC[C@@]23CCCN2C[C@H](F)C3)nc2c(F)c(Br)c(Cl)cc12]

To a solution of crude tert-butyl 8-(7-bromo-2,6-dichloro-8-fluoro quinazolin-4-yl)-3,8-diazabicyclo[3.2.1]octane-3-carboxylate (11.35 g, 22.43 mmol) from last step in DMSO (200 mL) was added KF (13.1 g, 224.3 mmol) and ((2R,7aS)-2-fluoro tetrahydro-1H-pyrrolizin-7a(5H)-yl)methanol (7.3 g, 44.86 mmol) at room temperature. The resulting mixture was stirred at 100° C. for about 2 days. After completion, the reaction mixture was diluted with ethyl acetate, washed with water and brine. The organic layer was dried over anhydrous Na₂SO₄, filtered and the filtrate was concentrated to give a residue. The residue was purified by silica gel column chromatography, eluted with gradient (PE to PE/EA=1/1 to DCM to DCM/MeOH=20/1 to MeOH ) to give the title product (3.71 g, 26%). MS (ESI, m/e) [M+H]⁺ 627.9.

### Step 2: tert-butyl 8-(7-(2-((tert-butoxycarbonyl)amino)-5,7-difluorobenzo[d]thiazol-4-yl)-6-chloro-8-fluoro-2-(((2R,7aS)-2-fluorotetrahydro-1H-pyrrolizin-7a(5H)-yl)methoxy) quinazolin-4-yl)-3,8-diazabicyclo[3.2.1]octane-3-carboxylate

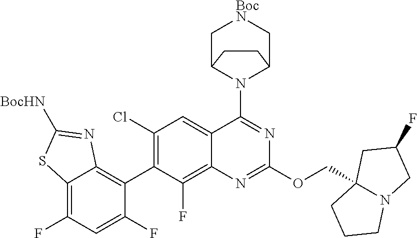 [SMILES: CC(C)(C)OC(=O)Nc1nc2c(-c3c(Cl)cc4c(N5C6CCC5CN(C(=O)OC(C)(C)C)C6)nc(OC[C@@]56CCCN5C[C@H](F)C6)nc4c3F)c(F)cc(F)c2s1]

To a solution of tert-butyl 8-(7-bromo-6-chloro-8-fluoro-2-(((2R,7aS)-2-fluorotetrahydro-1H-pyrrolizin-7a(5H)-yl)methoxy)quinazolin-4-yl)-3,8-diazabicyclo [3.2.1]octane-3-carboxylate (102 mg, 0.16 mmol) in toluene/H₂O/dioxane (15 mL/3 mL/15 mL) was subsequentially added Ruphos-Pd-G₃ (26 mg, 0.032 mmol), (2-((tert-butoxycarbonyl) amino)-5,7-difluorobenzo[d]thiazol-4-yl)boronic acid (112 mg, 0.34 mmol), Ruphos (39 mg, 0.084 mmol) and K₂CO₃ (215 mg, 1.56 mmol) at room temperature. The solution was stirred at 100° C. for overnight. After cooled to ambident temperature, the mixture was filtered via a pad of celite and washed with ethyl acetate. Then, silical gel was added to the solution and the solvent was removed in vacuo. The residual was purified by chromatography (the general eluent gradient: 100% DCM to DCM/MeOH=1/1 to MeOH) to give the crude product (135 mg, 27%). MS (ESI, m/e) [M+H]⁺ 834.1.

### Step 4: 4-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-5,7 difluorobenzo[d] thiazol-2-amine

To a solution of crude tert-butyl 8-(7-(2-((tert-butoxycarbonyl)amino)-5,7-difluorobenzo[d]thiazol-4-yl)-6-chloro-8-fluoro-2-(((2R,7aS)-2-fluorotetrahydro-1H-pyrrolizin-7a(5H)-yl) methoxy) quinazolin-4-yl)-3,8-diazabicyclo[3.2.1]octane-3-carboxylate (135 mg) in DCM (20 mL) was added TFA (4 mL) at room temperature. The mixture was stirred at room temperature for overnight and then add more TFA (1 mL). The solution was stirred at room temperature for further 5 h. Then, the mixture was evaporated in vacuo. The residue was redissolved in MeOH and 7 M NH₃ in MeOH was added to adjust PH >7. Then, the solvent was removed in vacuo and the residue was purified by Pre-HPLC to give the title product (0.41 mg, 0.31% for 2 steps, FA salt). 1H NMR (500 MHz, CD₃OD) δ 8.44 (s, 1H), 7.98 (s, 1H), 6.92 (t, J=9.7 Hz, 1H), 5.46 (d, J=52.6 Hz, 1H), 5.03 (m, 3H), 4.52 (ddd, J=33.0, 15.3, 11.2 Hz, 2H), 3.78-3.59 (m, 4H), 3.45 (m, 1H), 3.06 (m, 2H), 2.58-2.41 (m, 2H), 2.31 (m, 1H), 2.24-1.99 (m, 7H). MS (ESI, m/e) [M+H]⁺ 634.1.

### Example 94: 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-((trifluoromethyl) thio)quinazolin-7-yl)-7-fluorobenzo[d]thiazol-2-amine

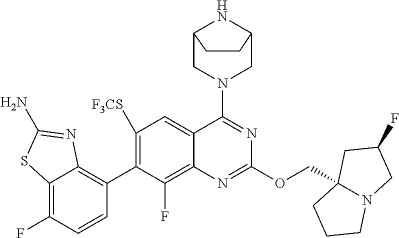

Synthetic Route

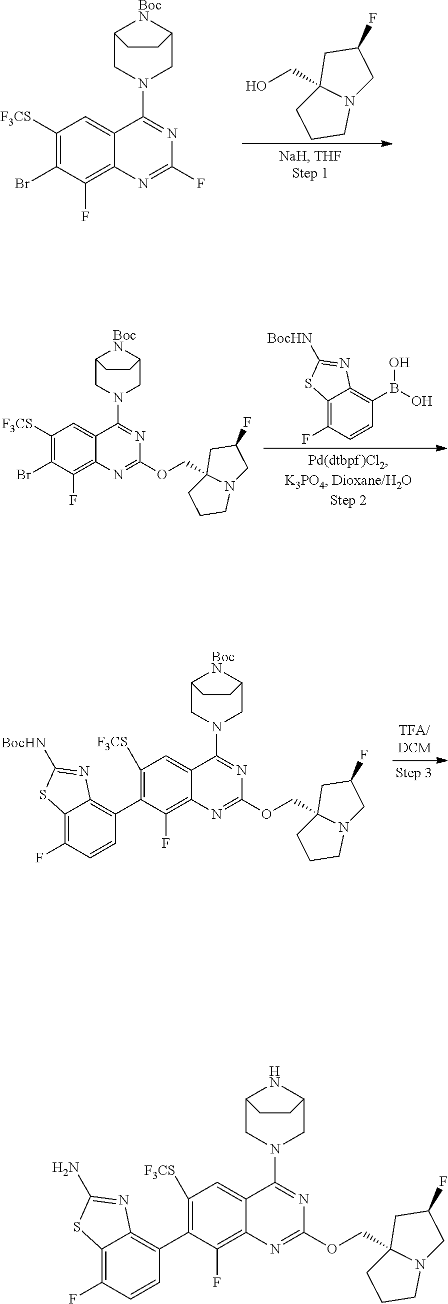

### Step 1: tert-butyl 3-(7-bromo-8-fluoro-2-(((2R,7aS)-2-fluorotetrahydro-1H-pyrrolizin-7a(5H)-yl)methoxy)-6-((trifluoromethyl)thio)quinazolin-4-yl)-3,8-diazabicyclo[3.2.1]octane-8-carboxylate

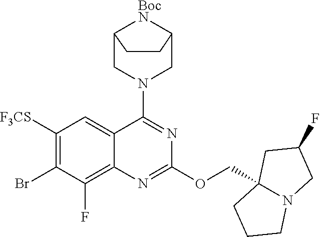

To a solution of ((2R,7aS)-2-fluorotetrahydro-1H-pyrrolizin-7a(5H)-yl) methanol (53.38 mg, 0.33 mmol) in THF (5 mL) was added sodium hydride (13.42 mg, 0.33 mmol) at room temperature. The resulting mixture was stirred at room temperature for 1 hrs. Then tert-butyl (1R,5S)-3-(7-bromo-2,8-difluoro-6-((trifluoromethyl)thio) quinazolin-4-yl)-3,8-diazabicyclo[3.2.1]octane-8-carboxylate (155 mg, 0.28 mmol) was added to the reaction mixture and stirred at room temperature for 1 hrs. After completion. The reaction mixture was diluted with EA (50 mL), washed with saturated NaCl (15 mL×3). The organic layer was dried over anhydrous Na₂SO₄, filtered and the filtrate was concentrated to give the residue. The residue was purified by Prep-TLC (PE:EA=3:1) to give the title product (150 mg, 0.22 mmol). MS (ESI, m/e) [M+H]⁺ 694.1, 696.1.

### Step 2: tert-butyl 3-(7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-((trifluoro methyl)thio)quinazolin-4-yl)-3,8-diazabicyclo[3.2.1]octane-8-carboxylate

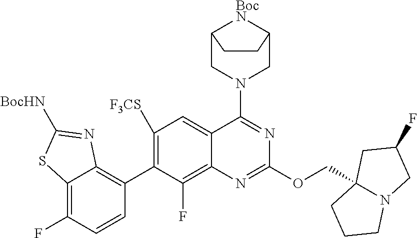

To a solution of tert-butyl 3-(7-bromo-8-fluoro-2-(((2R,7aS)-2-fluorotetrahydro-1H-pyrrolizin-7a(5H)-yl)methoxy)-6-((trifluoromethyl)thio)quinazolin-4-yl)-3,8-diazabicyclo [3.2.1]octane-8-carboxylate (150 mg, 0.22 mmol), (2-amino-7-fluorobenzo[d]thiazol-4-yl) boronic acid (93.6 mg, 0.3 mmol) and Pd(dtbpf)Cl₂ (12.9 mg, 0.02 mmol) in dioxane (5 mL) and H₂O (1 mL) was added K₃PO₄ (127.2 mg, 0.60 mmol) at room temperature. The resulting mixture was stirred at 100° C. for 16 hrs. After completion. The reaction mixture was diluted with EA (30 mL), washed with saturated NaCl (250 mL×3). The organic layer was dried over anhydrous Na₂SO₄, filtered and the filtrate was concentrated to give the residue. The residue was purified by Prep-TLC (pure EA) to give the title product (150 mg, 0.17 mmol), MS (ESI, m/e) [M+H]⁺ 882.3.

### Step 3: 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-((trifluoromethyl) thio)quinazolin-7-yl)-7-fluorobenzo[d]thiazol-2-amine

To a solution of tert-butyl 3-(7-(2-((tert-butoxycarbonyl)amino)-7-fluorobenzo[d]thiazol-4-yl)-8-fluoro-2-(((2R,7aS)-2-fluorotetrahydro-1H-pyrrolizin-7a(5H)-yl)methoxy)-6-((trifluoromethyl)thio)quinazolin-4-yl)-3,8-diazabicyclo[3.2.1] octane-8-carboxylate (150 mg, 0.17 mmol) in DCM (5 mL) was added TFA (5 mL) at room temperature. The resulting mixture was stirred at room temperature for 3 hrs. After completion. The reaction mixture was concentrated to give the residue. The residue was purified by High-HPLC to give the title product (52.44 mg). ¹H NMR (500 MHz, DMSO-d₆) δ 8.17 (s, 1H), 7.92 (s, 2H), 7.27-7.24 (m, 1H), 7.10-7.06 (m, 1H), 5.37-5.26 (m, 1H), 4.42-4.33 (m, 2H), 4.18-4.14 (m, 4H), 3.80-3.71 (m, 2H), 3.18-3.15 (m, 3H), 2.90-2.87 (m, 1H), 2.16-2.03 (m, 3H), 1.92-1.81 (m, 7H). MS (ESI, m/e) [M+1]⁺681.9.

### Example 95: 4-(4-((1S,4R)-2-((3,3-difluoropropyl) amino)-7-azabicyclo [2.2.1] heptan-7-yl)-8-fluoro-2-(((2R,7As)-2-fluorotetrahydro-1H-pyrrolizin-7a(5H)-yl) methoxy)-6-(trifluoromethyl) quinazolin-7-yl)-7-fluorobenzo[d]thiazol-2-amine

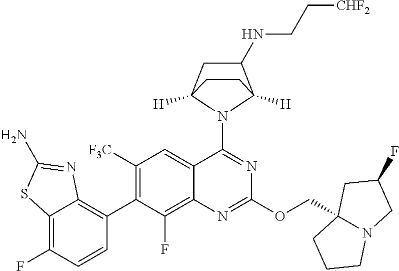 [SMILES: Nc1nc2c(-c3c(C(F)(F)F)cc4c(N5[C@@H]6CC[C@H]5C(NCCC(F)F)C6)nc(OC[C@@]56CCCN5C[C@H](F)C6)nc4c3F)ccc(F)c2s1]

Synthetic Route

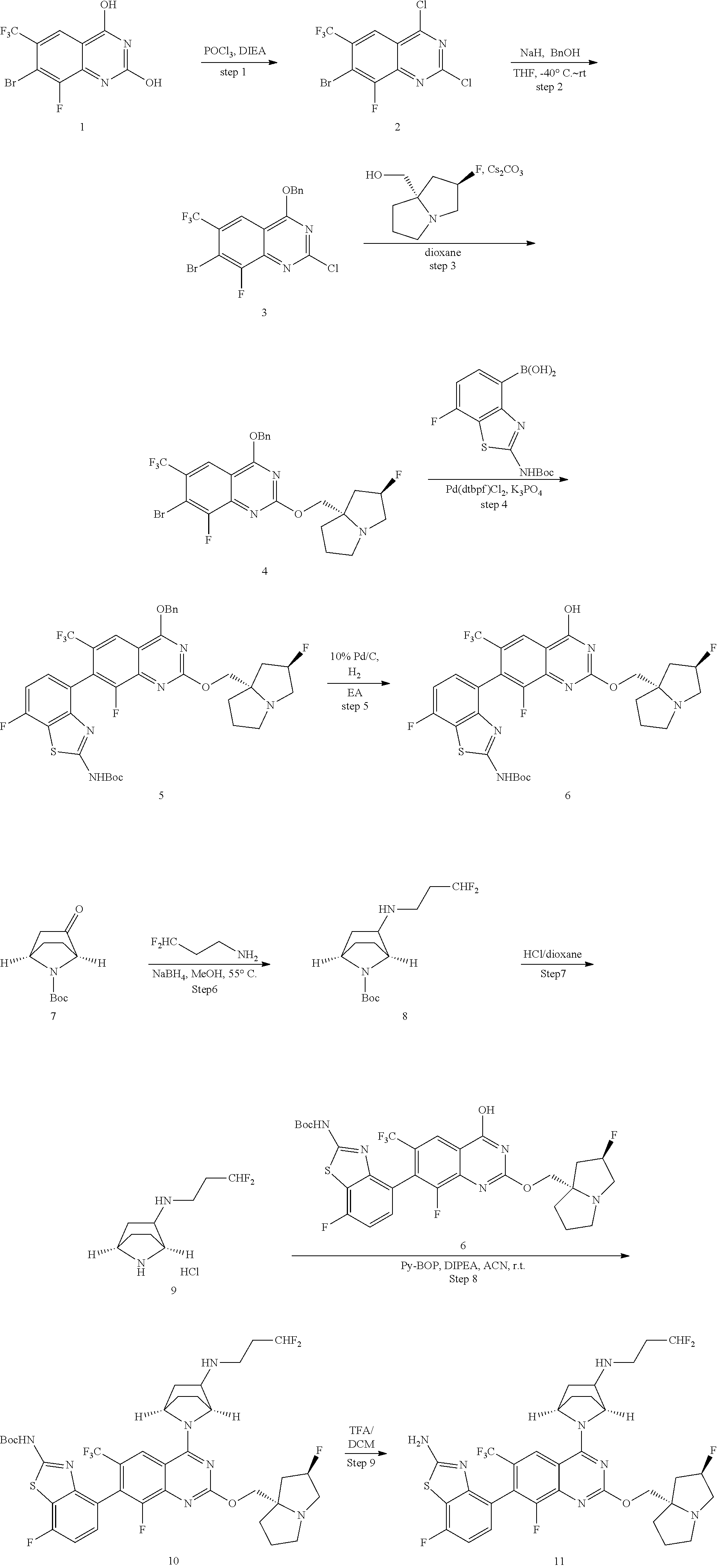

### Step 1: 7-bromo-2,4-dichloro-8-fluoro-6-(trifluoromethyl)quinazoline

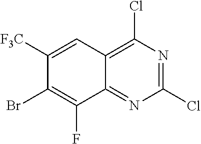 [SMILES: Fc1c(Br)c(C(F)(F)F)cc2c(Cl)nc(Cl)nc12]

To a stirred the solution of 7-bromo-8-fluoro-6-(trifluoromethyl)quinazoline-2,4-diol (20.0 g, 61.2 mmol) in 120 mL POCl₃ was added N,N-diisopropylethylamine (40.0 mL). The resulting mixture was stirred at 100° C. for 2 h. The cooled reaction mixture was evaporated under vacuo. The residue was purified by flash chromatography column (PE/DCM=1/1) to give the title compound (16.5 g, 74%). MS (ESI, m/e) [M+H]⁺ 364.7.

### Step 2: 4-(benzyloxy)-7-bromo-2-chloro-8-fluoro-6-(trifluoromethyl)quinazoline

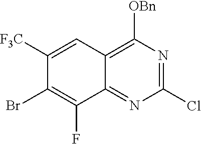 [SMILES: Fc1c(Br)c(C(F)(F)F)cc2c(OCc3ccccc3)nc(Cl)nc12]

To a stirred the solution of NaH (2.20 g, 54.4 mmol, 60%) in 50 mL THF was added BnOH (5.15 g, 47.6 mmol) at 0° C. and it was stirred for 30 min at 0° C. under N₂. Then, to the stirred solution of 7-bromo-2,4-dichloro-8-fluoro-6-(trifluoromethyl)quinazoline (16.5 g, 45.3 mmol) in 150 mL THF was added the above mixture at −40° C. The reaction mixture was stirred for 3 h at room temperature. The reaction was quenched with water and extracted with EA. The organic layer was combined, dried over sodium sulfate and evaporated. The residue was purified by flash chromatography column to give the title product (16.7 g, 84%). MS (ESI, m/e) [M+H]⁺ 436.7.

### Step 3: 4-(benzyloxy)-7-bromo-8-fluoro-2-(((2R,7aS)-2-fluorotetrahydro-1H-pyrrolizin-7a(5H)-yl)methoxy)-6-(trifluoromethyl)quinazoline

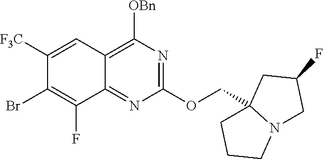

To a stirred solution of 4-(benzyloxy)-7-bromo-2-chloro-8-fluoro-6-(trifluoro methyl)quinazoline (12.5 g, 28.7 mmol) in 120 mL dioxane was added Cs₂CO₃ (23.4 g, 71.8 mmol) and ((2R,7aS)-2-fluorotetrahydro-1H-pyrrolizin-7a(5H)-yl)methanol (6.85 g, 43.1 mmol). The resulting mixture was stirred for overnight at 80° C. under N₂. The cooled reaction mixture was filtered and filtrate was concentrated under reduced pressure. The residue was purified by flash chromatography column (eluting with DCM/MeOH=20/1) give the title product (13.1 g, 82%). MS (ESI, m/e) [M+H]⁺ 557.8, 559.9.

### Step 4: tert-butyl (4-(4-(benzyloxy)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-yl) carbamate

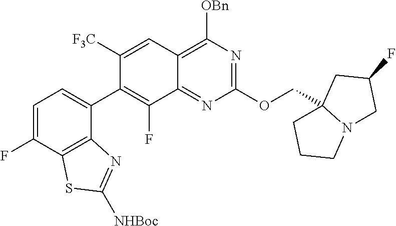

To a 250 mL round-bottomed flask was added 4-(benzyloxy)-7-bromo-8-fluoro-2-(((2R,7aS)-2-fluorotetrahydro-1H-pyrrolizin-7a(5H)-yl)methoxy)-6-(trifluoromethyl)quinazoline (10.0 g, 17.9 mmol), (2-((tert-butoxycarbonyl)amino)-7-fluorobenzo[d]thiazol-4-yl)boronic acid (11.2 g, 35.8 mmol), K₃PO₄ (15.2 g, 71.6 mmol), Pd(dtbpf)Cl₂ (1.17 g, 1.79 mmol), dioxane (120 mL) and H₂O (24 mL). The reaction mixture was degassed with N₂ for 3 times and stirred at 100° C. for 3 hours. The resulting cooled mixture was concentrated and purified by flash column chromatography (DCM/MeOH=20/1) to give the title product (8.67 g, 65%). MS (ESI, m/e) [M+H]⁺ 745.9.

### Step 5: tert-butyl (7-fluoro-4-(8-fluoro-2-(((2R,7aS)-2-fluorotetrahydro-1H-pyrrolizin-7a(5H)-yl)methoxy)-4-hydroxy-6-(trifluoromethyl)quinazolin-7-yl)benzo[d]thiazol-2-yl)carbamate

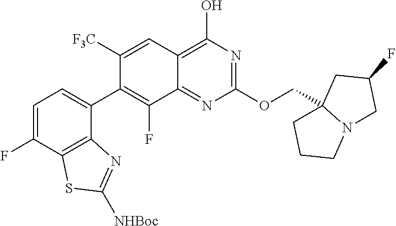

To a stirred solution of tert-butyl (4-(4-(benzyloxy)-8-fluoro-2-(((2R,7aS)-2-fluorotetrahydro-1H-pyrrolizin-7a(5H)-yl)methoxy)-6-(trifluoromethyl)quinazolin-7-yl)-7-fluoro benzo[d]thiazol-2-yl)carbamate (8.67 g, 11.6 mmol) in 120 mL EA was added 10% Pd/C (2.90 g) under N₂. The resulting mixture was degassed with H₂ for 3 times and stirred for 5 h at room temperature under H₂ atmosphere. The reaction mixture was filtered and filtrate was concentrated under reduced pressure. The residue was purified by flash chromatography column (eluting with DCM/MeOH=10/1) to give the title product (6.54 g, 86%). MS (ESI, m/e) [M+1]⁺656.3.

### Step 6: tert-butyl (1S,4R)-2-((3,3-difluoropropyl) amino)-7-azabicyclo [2.2.1] heptane-7-carboxylate

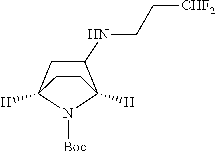 [SMILES: CC(C)(C)OC(=O)N1[C@@H]2CC[C@H]1C(NCCC(F)F)C2]

To a solution of tert-butyl (1S,4R)-2-oxo-7-azabicyclo [2.2.1] heptane-7-carboxylate (300 mg, 1.42 mmol) in MeOH (20 mL) was added 3,3-difluoropropan-1-amine (233 mg, 1.775 mmol) at room temperature, and the mixture was stirred at 55° C. for overnight. Upon completion, the mixture was cooled to room temperature and NaBH₄ (329 mg, 5.68 mmol) was added. The resulting mixture was stirred at room temperature for 1 h. Solvents were concentrated, and the residue was diluted with water (20 mL). Aqueous layer was extracted with DCM (30 mL *3), and the combined organic layer was concentrated to give a residue as the title compound (416 mg). MS (ESI, m/e) [M+1]⁺291.

### Step 7: (1S,4R)—N-(3,3-difluoropropyl)-7-azabicyclo [2.2.1] heptan-2-amine

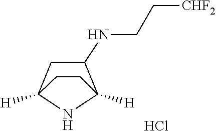 [SMILES: FC(F)CCNC1C[C@H]2CC[C@@H]1N2 ; Cl]

To a solution of tert-butyl (1S,4R)-2-((3,3-difluoropropyl) amino)-7-aza bicyclo[2.2.1]heptane-7-carboxylate (416 mg, 1.43 mmol) in DCM (10 mL) was added HCl/dioxane (15 mL) at room temperature, and the mixture was stirred at room temperature for 1 h. The resulting mixture was concentrated to give a residue as the title compound (382 mg). MS (ESI, m/e) [M+H]⁺ 191.

### Step 8: tert-butyl (4-(4-((1S,4R)-2-((3,3-difluoropropyl) amino)-7-aza bicyclo [2.2.1]heptan-7-yl)-8-fluoro-2-(((2R,7aS)-2-fluorotetrahydro-1H-pyrrolizin-7a(5H)-yl) methoxy)-6-(trifluoromethyl) quinazolin-7-yl)-7-fluorobenzo[d]thiazol-2-yl) carbamate

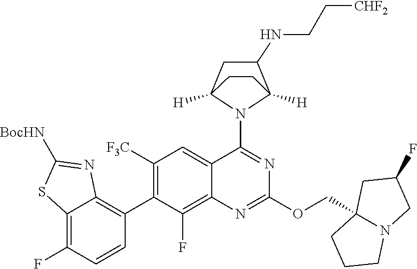 [SMILES: CC(C)(C)OC(=O)Nc1nc2c(-c3c(C(F)(F)F)cc4c(N5[C@@H]6CC[C@H]5C(NCCC(F)F)C6)nc(OC[C@@]56CCCN5C[C@H](F)C6)nc4c3F)ccc(F)c2s1]

To a solution of tert-butyl (7-fluoro-4-(8-fluoro-2-(((2R,7aS)-2-fluorotetra hydro-1H-pyrrolizin-7a(5H)-yl)methoxy)-4-hydroxy-6-(trifluorom ethyl)quinazolin-7yl) benzo[d]thiazol-2-yl)carbamate (65 mg, 0.1 mmol) in ACN (15 mL) was added (1S,4R)—N-(3,3-difluoropropyl)-7-azabicyclo [2.2.1] heptan-2-amine (56.6 mg, 0.25 mmol), benzotriazole-1-yl-oxytripyrrolidinophosphonium hexafluorophosphate (78 mg, 0.15 mmol) and DIPEA (77.4 mg, 0.6 mmol) at room temperature, and the mixture was stirred at room temperature for overnight. Solvent was concentrated and the residue was purified by chromatography column on silica (eluting with DCM/MeOH=25/1) to give the title product (61 mg). MS (ESI, m/e) [M+H]⁺ 828.

### Step 9: 4-(4-((1S,4R)-2-((3,3-difluoropropyl) amino)-7-azabicyclo [2.2.1] heptan-7-yl)-8-fluoro-2-(((2R,7aS)-2-fluorotetrahydro-1H-pyrrolizin-7a(5H)-yl) methoxy)-6-(trifluoromethyl) quinazolin-7-yl)-7-fluorobenzo[d]thiazol-2-amine

To a solution of tert-butyl (4-(4-((1S,4R)-2-((3,3-difluoropropyl) amino)-7-azabicyclo [2.2.1] heptan-7-yl)-8-fluoro-2-(((2R,7aS)-2-fluorotetrahydro-1H-pyrrolizin-7a(5H)-yl) methoxy)-6-(trifluoromethyl) quinazolin-7-yl)-7-fluorobenzo[d]thiazol-2-yl) carbamate (61 mg, 0.074 mmol) in DCM (6 mL) was added TFA (6 mL) at room temperature, and the mixture was stirred at room temperature for 1 h. The resulting mixture was concentrated and pH was adjusted to 7 with Na₂CO₃. The organic layer was separated and solvents were removed to give a residue which was further purified by Prep-HPLC to give title product (12.14 mg, FA salt). ¹H NMR (500 MHz, DMSO-d₆) δ 8.15-8.14 (m, 1H), 7.92-7.88 (s, 2H), 7.23-7.16 (m, 1H), 7.07-7.05 (m, 1H), 6.54-6.09 (m, 1H), 5.50-5.40 (m, 1H), 4.91-4.84 (m, 2H), 4.52-4.38 (s, 2H), 3.55-3.35 (m, 5H), 3.11-2.85 (m, 4H), 2.37-1.66 (m, 13H). MS (ESI, m/e) [M+H]⁺ 728.

### Example 96: 6-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl) quinazolin-7-yl)-4-methyl-5-(trifluoromethyl)pyridin-2-amine

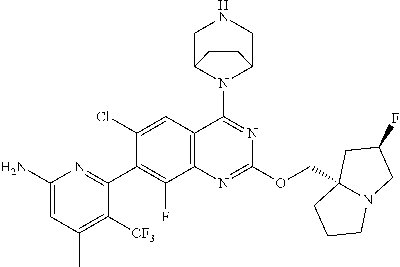 [SMILES: Cc1cc(N)nc(-c2c(Cl)cc3c(N4C5CCC4CNC5)nc(OC[C@@]45CCCN4C[C@H](F)C5)nc3c2F)c1C(F)(F)F]

Example 96 was prepared by similar procedure as described in Example 35 by replacing tert-butyl 3,8-diazabicyclo[3.2.1]octane-8-carboxylate with tert-butyl 3,8-diazabicyclo [3.2.1]octane-3-carboxylate to give the title product (6.5 mg). ¹H NMR (500 MHz, DMSO-d₆) δ 7.83 (s, 1H), 6.85 (s, 2H), 6.49 (s, 1H), 5.32-5.22 (m, 1H), 4.82-4.77 (m, 2H), 4.07-3.96 (m, 2H), 3.32-3.08 (m, 6H), 2.84-2.78 (m, 3H), 2.37 (s, 3H), 2.12-1.76 (m, 10H). MS (ESI, m/e) [M+H]⁺ 623.9.

### Example 97: 7-fluoro-4-(8-fluoro-2-(((2R,7aS)-2-fluorotetrahydro-1H-pyrrolizin-7a(5H)-yl) methoxy)-4-((1S,4R)-2-((3-methoxypropyl) amino)-7-azabicyclo [2.2.1] heptan-7-yl)-6-(trifluoromethyl) quinazolin-7-yl) benzo[d]thiazol-2-amine

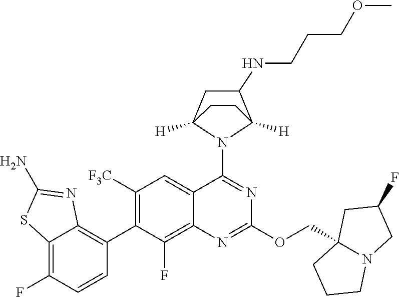 [SMILES: COCCCNC1C[C@H]2CC[C@@H]1N2c1nc(OC[C@@]23CCCN2C[C@H](F)C3)nc2c(F)c(-c3ccc(F)c4sc(N)nc34)c(C(F)(F)F)cc12]

Synthetic Route

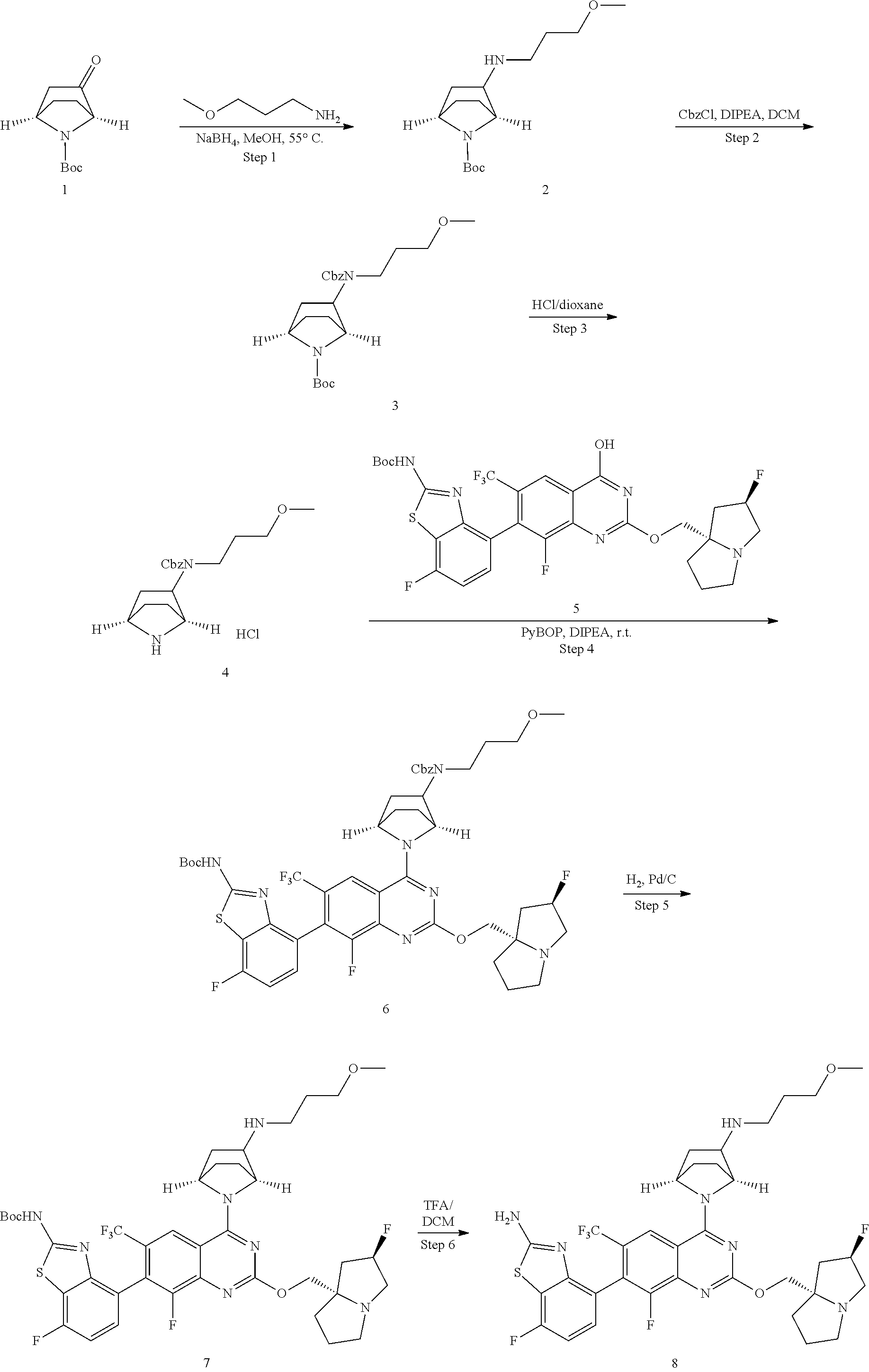

### Step 1: tert-butyl (1S,4R)-2-((3-methoxypropyl) amino)-7-azabicyclo [2.2.1] heptane-7-carboxylate

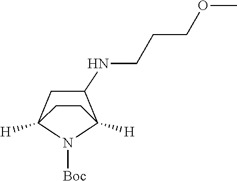 [SMILES: COCCCNC1C[C@H]2CC[C@@H]1N2C(=O)OC(C)(C)C]

To a solution of tert-butyl (1S,4R)-2-oxo-7-azabicyclo [2.2.1] heptane-7-carboxylate (211 mg, 1 mmol) in MeOH (15 mL) was added 3-methoxypropan-1-amine (111 mg, 1.25 mmol) at room temperature, and the mixture was stirred at 55° C. for overnight. Upon completion, the mixture was cooled to room temperature and NaBH₄ (232 mg, 4 mmol) was added. The resulting mixture was stirred at room temperature for 1 h. Solvents were concentrated, and the residue was diluted with water (20 mL). Aqueous layer was extracted with DCM (30 mL *3), and the combined organic layer was concentrated to give a residue as the title compound (275 mg). MS (ESI, m/e) [M+H]⁺ 285.

### Step 2: tert-butyl (1S,4R)-2-(((benzyloxy) carbonyl) (3-methoxypropyl) amino)-7-azabicyclo [2.2.1] heptane-7-carboxylate

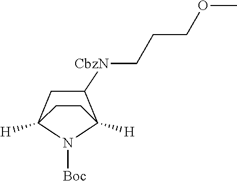 [SMILES: COCCCN(C(=O)OCc1ccccc1)C1C[C@H]2CC[C@@H]1N2C(=O)OC(C)(C)C]

To a solution of tert-butyl (1S,4R)-2-((3-methoxypropyl) amino)-7-aza bicyclo [2.2.1]heptane-7-carboxylate (275 mg, 0.968 mmol) in DCM (20 mL) was added benzyl carbonochloridate (181.65 mg, 1.065 mmol) and DIPEA (250 mg, 1.936 mmol) at 0° C., and the mixture was stirred at room temperature for 1 h. The resulting mixture was quenched with water (20 mL) and extracted with DCM (30 mL *3), and the combined organic layer was concentrated to give a crude (421 mg) which was not purified and directly used next step. MS (ESI, m/e) [M+H]⁺ 419.

### Step 3: benzyl ((1S,4R)-7-azabicyclo [2.2.1] heptan-2-yl) (3-methoxy propyl) carbamate

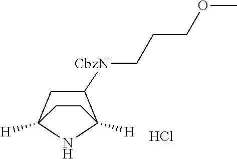 [SMILES: COCCCN(C(=O)OCc1ccccc1)C1C[C@H]2CC[C@@H]1N2 ; Cl]

To a solution of tert-butyl (1S,4R)-2-(((benzyloxy) carbonyl) (3-methoxy propyl) amino)-7-azabicyclo [2.2.1] heptane-7-carboxylate (421 mg, 1.0 mmol) in DCM (6 mL) was added HCl/dioxane (12 mL) at room temperature, and the mixture was stirred at room temperature for 1 h. The resulting mixture was concentrated to give a residue as the title compound (406 mg). MS (ESI, m/e) [M+H]⁺ 319.

### Step 4: benzyl ((1S,4R)-7-(7-(2-((tert-butoxycarbonyl) amino)-7-fluoro benzo[d]thiazol-4-yl)-8-fluoro-2-(((2R,7aS)-2-fluorotetrahydro-1H-pyrrolizin-7a(5H)-yl) methoxy)-6-(trifluoromethyl) quinazolin-4-yl)-7-azabicyclo [2.2.1] heptan-2-yl) (3-methoxypropyl) carbamate

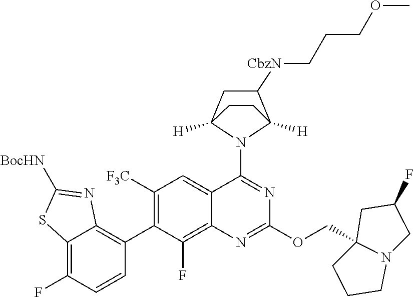 [SMILES: COCCCN(C(=O)OCc1ccccc1)C1C[C@H]2CC[C@@H]1N2c1nc(OC[C@@]23CCCN2C[C@H](F)C3)nc2c(F)c(-c3ccc(F)c4sc(NC(=O)OC(C)(C)C)nc34)c(C(F)(F)F)cc12]

To a solution of tert-butyl (7-fluoro-4-(8-fluoro-2-(((2R,7aS)-2-fluorotetra hydro-1H-pyrrolizin-7a(5H)-yl)methoxy)-4-hydroxy-6-(trifluoromethyl)quinazolin-7-yl)benzo[d]thiazol-2-yl)carbamate (100 mg, 0.152 mmol) in ACN (15 mL) was added (benzyl ((1S,4R)-7-azabicyclo [2.2.1]heptan-2-yl) (3-methoxypropyl) carbamate (135.5 mg, 0.381 mmol), benzotriazole-1-yl-oxytripyrrolidinophosphonium hexafluorophosphate (118.6 mg, 0.228 mmol) and DIPEA (118 mg, 0.912 mmol) at room temperature, and the mixture was stirred at room temperature for overnight. Solvent was concentrated and the residue was purified by chromatography column on silica (eluting with DCM/MeOH=25/1) to give the title product (116 mg). MS (ESI, m/e) [M+H]⁺ 956.

### Step 5: tert-butyl (7-fluoro-4-(8-fluoro-2-(((2R,7aS)-2-fluorotetrahydro-1H-pyrrolizin-7a(5H)-yl) methoxy)-4-((1S,4R)-2-((3-methoxypropyl) amino)-7-azabicyclo [2.2.1] heptan-7-yl)-6-(trifluoromethyl) quinazolin-7-yl) benzo[d]thiazol-2-yl) carbamate

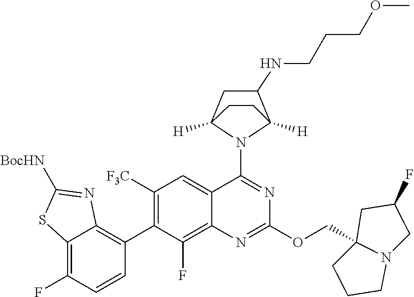 [SMILES: COCCCNC1C[C@H]2CC[C@@H]1N2c1nc(OC[C@@]23CCCN2C[C@H](F)C3)nc2c(F)c(-c3ccc(F)c4sc(NC(=O)OC(C)(C)C)nc34)c(C(F)(F)F)cc12]

To a solution of benzyl ((1S,4R)-7-(7-(2-((tert-butoxycarbonyl) amino)-7-fluorobenzo[d]thiazol-4-yl)-8-fluoro-2-(((2R,7aS)-2-fluorotetrahydro-1H-pyrrolizin-7a(5H)-yl) methoxy)-6-(trifluoromethyl) quinazolin-4-yl)-7-azabicyclo [2.2.1] heptan-2-yl) (3-methoxypropyl) carbamate (116 mg, 0.12 mmol) in MeOH (20 mL) was added Pd/C (60 mg) at room temperature, and the mixture was stirred under hydrogen at room temperature for 3 h. The resulting mixture was concentrated to give a residue (87 mg). MS (ESI, m/e) [M+H]⁺ 822.

### Step 6: 7-fluoro-4-(8-fluoro-2-(((2R,7aS)-2-fluorotetrahydro-1H-pyrrolizin-7a(5H)-yl) methoxy)-4-((1S,4R)-2-((3-methoxypropyl) amino)-7-azabicyclo [2.2.1] heptan-7-yl)-6-(trifluoromethyl) quinazolin-7-yl) benzo[d]thiazol-2-amine

To a solution of tert-butyl (7-fluoro-4-(8-fluoro-2-(((2R,7aS)-2-fluorotetra hydro-1H-pyrrolizin-7a(5H)-yl) methoxy)-4-((1S,4R)-2-((3-methoxypropyl) amino)-7-azabicyclo [2.2.1] heptan-7-yl)-6-(trifluoromethyl) quinazolin-7-yl) benzo[d]thiazol-2-yl) carbamate (87 mg, 0.106 mmol) in DCM (6 mL) was added TFA (4 mL) at room temperature, and the mixture was stirred at room temperature for 1 h. The resulting mixture was concentrated and pH was adjusted to 7 with Na₂CO₃. The organic layer was separated and solvents were removed to give a residue which was further purified by Prep-HPLC to give title product (30.21 mg). ¹H NMR (500 MHz, CD₃OD) δ 8.23 (s, 1H), 7.24-7.14 (m, 1H), 7.00-6.96 (m, 1H), 5.56-5.45 (m, 1H), 5.13-4.97 (m, 2H), 4.62-4.59 (m, 2H), 3.81-3.69 (m, 4H), 3.54-3.52 (m, 2H), 3.40-3.33 (m, 4H), 3.09-3.05 (m, 2H), 2.65-2.51 (m, 3H), 2.37-2.35 (m, 1H), 2.27-2.25 (m, 2H), 2.18-2.02 (m, 4H), 2.00-1.92 (m, 2H), 1.83-1.81 (m, 1H), 1.55-1.53 (m, 1H). MS (ESI, m/e) [M+H]⁺ 721.9.

### Example 98: 4-(4-(3,9-diazabicyclo[3.3.1]nonan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

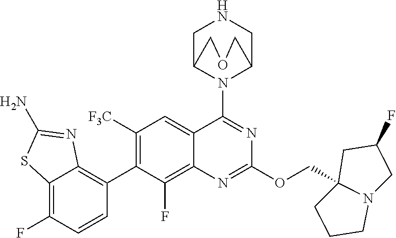 [SMILES: Nc1nc2c(-c3c(C(F)(F)F)cc4c(N5C6CNCC5COC6)nc(OC[C@@]56CCCN5C[C@H](F)C6)nc4c3F)ccc(F)c2s1]

Synthetic Route

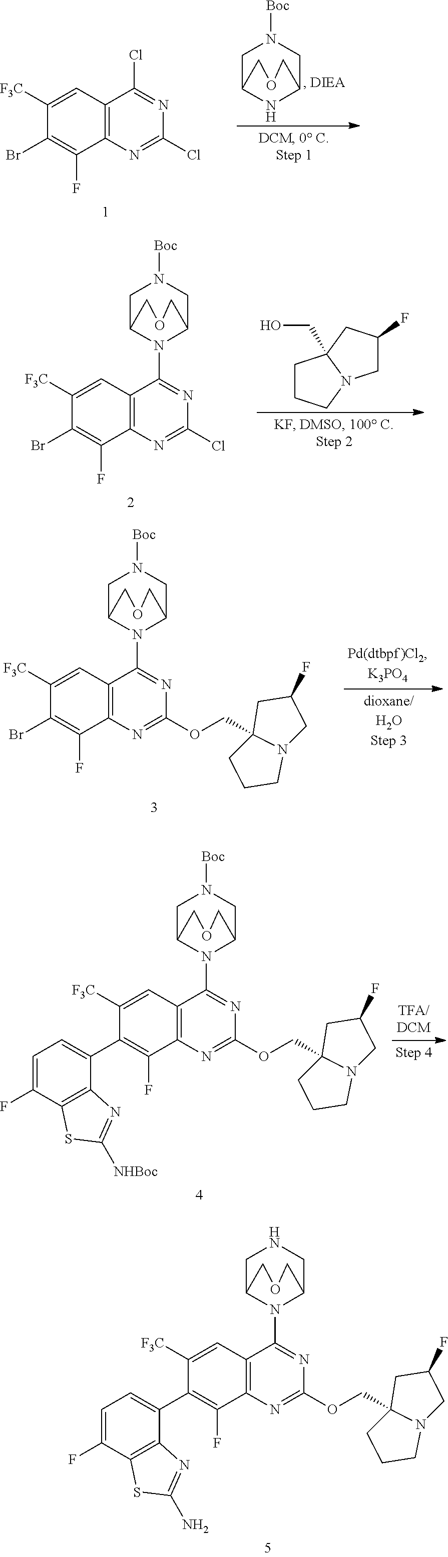

### Step 1: tert-butyl 9-(7-bromo-2-chloro-8-fluoro-6-(trifluoromethyl) quinazolin-4-yl)-3-oxa-7,9-diazabicyclo[3.3.1]nonane-7-carboxylate

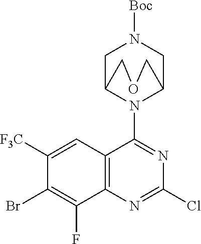 [SMILES: CC(C)(C)OC(=O)N1CC2COCC(C1)N2c1nc(Cl)nc2c(F)c(Br)c(C(F)(F)F)cc12]

To a solution of 7-bromo-2,4-dichloro-8-fluoro-6-(trifluoromethyl) quinazoline (500 mg, 1.4 mmol) in DCM (8 mL) was added DIEA (543 mg, 4.2 mmol), then to the above mixture was added the solution of tert-butyl 3-oxa-7,9-diazabicyclo [3.3.1]nonane-7-carboxylate (320 mg, 1.4 mmol) in DCM dropwise at 0° C. The reaction mixture was stirred for 1 h at 0° C. The resulting mixture was concentrated and purified by flash column chromatography (eluting with PE/EA=5/1) to give the desired product (700 mg, 90%). MS (ESI, m/e) [M+H]⁺ 556.8.

### Step 2: tert-butyl 9-(7-bromo-8-fluoro-2-(((2R,7aS)-2-fluorotetrahydro-1H-pyrrolizin-7a(5H)-yl)methoxy)-6-(trifluoromethyl)quinazolin-4-yl)-3-oxa-7,9-diazabicyclo[3.3.1]nonane-7-carboxylate

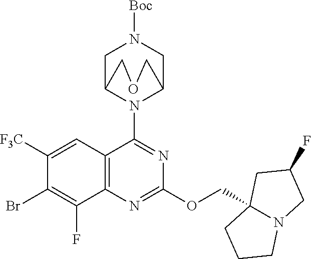 [SMILES: CC(C)(C)OC(=O)N1CC2COCC(C1)N2c1nc(OC[C@@]23CCCN2C[C@H](F)C3)nc2c(F)c(Br)c(C(F)(F)F)cc12]

To a solution of tert-butyl 9-(7-bromo-2-chloro-8-fluoro-6-(trifluoro methyl)quinazolin-4-yl)-3-oxa-7,9-diazabicyclo[3.3.1]nonane-7-carboxylate (500 mg, 0.9 mmol) in DMSO (10 mL) was added KF (314 mg, 5.4 mmol) and ((2R,7aS)-2-fluoro tetrahydro-1H-pyrrolizin-7a(5H)-yl)methanol (430 mg, 2.7 mmol), the resulting mixture was stirred for overnight at 100° C. The resulting cooled mixture was extracted with EA, combined organic phases and concentrated under reduced pressure. The crude was purified by flash column chromatography on silica (eluting with DCM/MeOH=20/1) to give the title product (490 mg, 80%). MS (ESI, m/e) [M+H]⁺ 677.9, 679.8.

### Step 3: tert-butyl 9-(7-(2-((tert-butoxycarbonyl)amino)-7-fluorobenzo[d] thiazol-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3-oxa-7,9-diazabicyclo[3.3.1]nonane-7-carboxylate

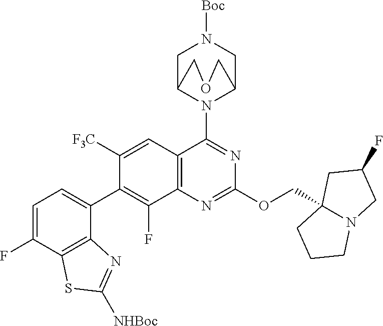 [SMILES: CC(C)(C)OC(=O)Nc1nc2c(-c3c(C(F)(F)F)cc4c(N5C6COCC5CN(C(=O)OC(C)(C)C)C6)nc(OC[C@@]56CCCN5C[C@H](F)C6)nc4c3F)ccc(F)c2s1]

To a mixture of tert-butyl 9-(7-bromo-8-fluoro-2-(((2R,7aS)-2-fluorotetra hydro-1H-pyrrolizin-7a(5H)-yl)methoxy)-6-(trifluoromethyl)quinazolin-4-yl)-3-oxa-7,9-diazabicyclo[3.3.1]nonane-7-carboxylate (490 mg, 0.72 mmol), (2-((tert-butoxycarbonyl) amino)-7-fluorobenzo[d]thiazol-4-yl)boronic acid (450 mg, 1.44 mmol), K₃PO₄ (610 mg, 2.88 mmol), Pd(dtbpf)Cl₂ (91 mg, 0.14 mmol) was added dioxane (10.0 mL) and H₂O (2.0 mL), the reaction mixture was stirred at 100° C. for 3 hours. The resulting cooled mixture was concentrated and purified by flash column chromatography (DCM/MeOH=20/1) to give the title product (435 mg, 70%). MS (ESI, m/e) [M+1]⁺865.9.

### Step 4: 4-(4-(3-oxa-7,9-diazabicyclo[3.3.1]nonan-9-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

To a solution of tert-butyl 9-(7-(2-((tert-butoxycarbonyl)amino)-7-fluoro benzo[d]thiazol-4-yl)-8-fluoro-2-(((2R,7aS)-2-fluorotetrahydro-1H-pyrrolizin-7a(5H)-yl) methoxy)-6-(trifluoromethyl)quinazolin-4-yl)-3-oxa-7,9-diazabicyclo[3.3.1]nonane-7-carboxylate (100 mg, 0.12 mmol) in DCM (6.0 mL) was added TFA (3.0 mL), the resulting mixture was stirred for 2 hour at room temperature. The resulting solution was washed with NaHCO₃/H₂O (Sat. 20 mL) and concentrated to give a residue which was further purified by Prep-HPLC to give the title product (14.5 mg, FA salt, 19%). ¹H NMR (500 MHz, CD₃OD) δ 8.50 (s, 1H), 8.01 (s, 1H), 7.22-7.15 (m, 1H), 6.97 (t, J=8.8 Hz, 1H), 5.38 (d, J=53.0 Hz, 1H), 4.64 (s, 2H), 4.48-4.34 (m, 2H), 4.32-4.20 (m, 4H), 3.70-3.55 (m, 4H), 3.40 (dt, J=18.1, 12.8 Hz, 3H), 3.21-3.10 (m, 1H), 2.49-2.28 (m, 2H), 2.26-2.17 (m, 1H), 2.09 (dd, J=17.1, 6.4 Hz, 2H), 1.97 (s, 1H). MS (ESI, m/e) [M+H]⁺ 666.4.

### Example 99: 3-(((1S,4R)-7-(7-(2-amino-7-fluorobenzo[d]thiazol-4-yl)-8-fluoro-2-(((2R,7aS)-2-fluorotetrahydro-1H-pyrrolizin-7a(5H)-yl) methoxy)-6-(trifluoro methyl) quinazolin-4-yl)-7-azabicyclo [2.2.1] heptan-2-yl) amino)-N,N-dimethyl propanamide

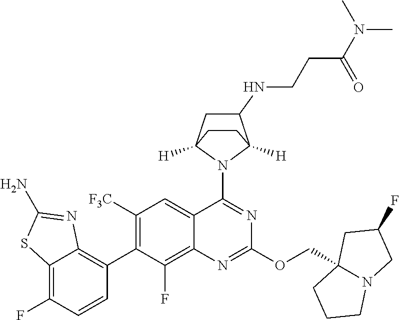 [SMILES: CN(C)C(=O)CCNC1C[C@H]2CC[C@@H]1N2c1nc(OC[C@@]23CCCN2C[C@H](F)C3)nc2c(F)c(-c3ccc(F)c4sc(N)nc34)c(C(F)(F)F)cc12]

Synthetic Route

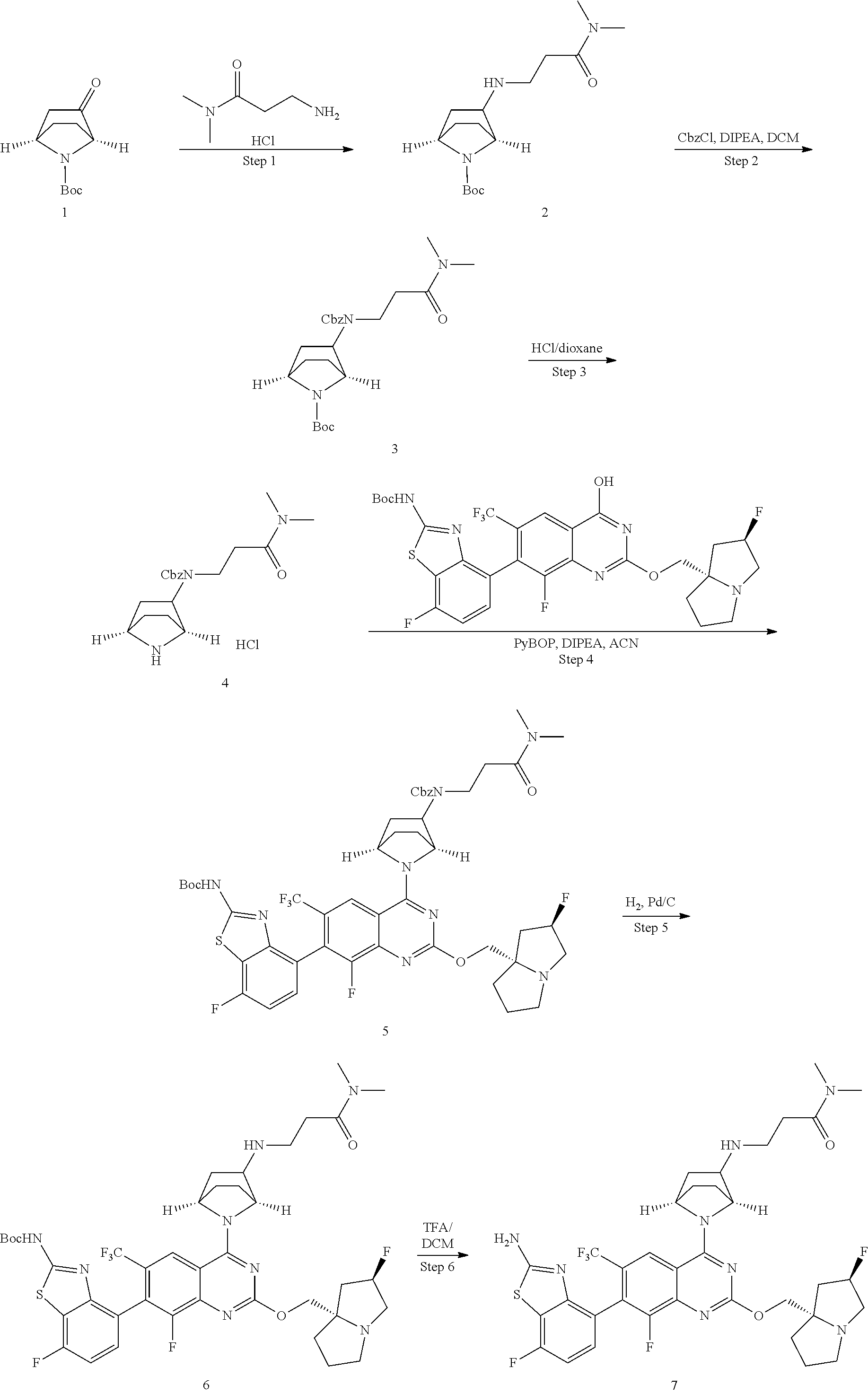

### Step 1: tert-butyl (1S,4R)-2-((3-(dimethylamino)-3-oxopropyl) amino)-7-azabicyclo [2.2.1]heptane-7-carboxylate

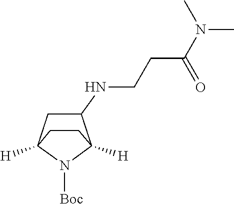 [SMILES: CN(C)C(=O)CCNC1C[C@H]2CC[C@@H]1N2C(=O)OC(C)(C)C]

To a solution of tert-butyl (1S,4R)-2-oxo-7-azabicyclo [2.2.1] heptane-7-carboxylate (211 mg, 1 mmol) in MeOH (15 mL) was added 3-amino-N,N-dimethyl propanamide (145 mg, 1.25 mmol) at room temperature, and the mixture was stirred at 55° C. for overnight. Upon completion, the mixture was cooled to room temperature and NaBH₄ (232 mg, 4 mmol) was added. The resulting mixture was stirred at room temperature for 1 h. Solvents were concentrated, and the residue was diluted with water (20 mL). Aqueous layer was extracted with DCM (30 mL *3), and the combined organic layer was concentrated to give a residue as the title compound (312 mg). MS (ESI, m/e) [M+H]⁺ 312.

### Step 2: tert-butyl (1S,4R)-2-(((benzyloxy)carbonyl) (3-(dimethylamino)-3-oxopropyl) amino)-7-azabicyclo [2.2.1] heptane-7-carboxylate

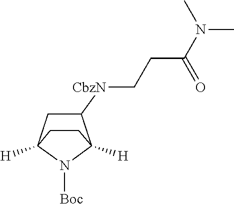 [SMILES: CN(C)C(=O)CCN(C(=O)OCc1ccccc1)C1C[C@H]2CC[C@@H]1N2C(=O)OC(C)(C)C]

To a solution of tert-butyl (1S,4R)-2-((3-(dimethylamino)-3-oxopropyl) amino)-7-azabicyclo [2.2.1] heptane-7-carboxylate (300 mg, 0.965 mmol) in DCM (20 mL) was added benzyl carbonochloridate (181.65 mg, 1.065 mmol) and DIPEA (250 mg, 1.936 mmol) at 0° C., and the mixture was stirred at room temperature for 1 h. The resulting mixture was quenched with water (20 mL) and extracted with DCM (30 mL *3), and the combined organic layer was concentrated to give a title crude product (452 mg) which was not purified and used next step. MS (ESI, m/e) [M+H]⁺ 446.

### Step 3: benzyl ((1S,4R)-7-azabicyclo [2.2.1] heptan-2-yl) (3-(dimethyl amino)-3-oxopropyl) carbamate

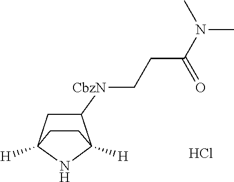 [SMILES: CN(C)C(=O)CCN(C(=O)OCc1ccccc1)C1C[C@H]2CC[C@@H]1N2 ; Cl]

To a solution of tert-butyl (1S,4R)-2-(((benzyloxy)carbonyl) (3-(dimethyl amino)-3-oxopropyl) amino)-7-azabicyclo [2.2.1] heptane-7-carboxylate (452 mg, 1.0 mmol) in DCM (5 mL) was added HCl/dioxane (8 mL) at room temperature, and the mixture was stirred at room temperature for 1 h. The resulting mixture was concentrated to give a residue as the title compound (462 mg). MS (ESI, m/e) [M+H]⁺ 382.

### Step 4: benzyl ((1S,4R)-7-(7-(2-((tert-butoxycarbonyl) amino)-7-fluoro benzo[d]thiazol-4-yl)-8-fluoro-2-(((2R,7aS)-2-fluorotetrahydro-1H-pyrrolizin-7a(5H)-yl) methoxy)-6-(trifluoromethyl) quinazolin-4-yl)-7-azabicyclo [2.2.1] heptan-2-yl) (3-(di methylamino)-3-oxopropyl) carbamate

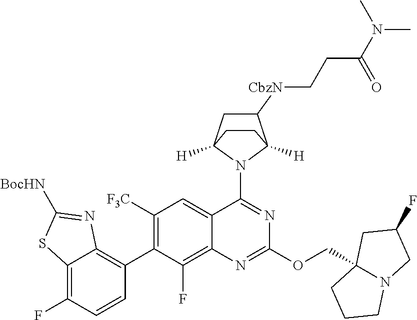 [SMILES: CN(C)C(=O)CCN(C(=O)OCc1ccccc1)C1C[C@H]2CC[C@@H]1N2c1nc(OC[C@@]23CCCN2C[C@H](F)C3)nc2c(F)c(-c3ccc(F)c4sc(NC(=O)OC(C)(C)C)nc34)c(C(F)(F)F)cc12]

To a solution of tert-butyl (7-fluoro-4-(8-fluoro-2-(((2R,7aS)-2-fluorotetra hydro-1H-pyrrolizin-7a(5H)-yl)methoxy)-4-hydroxy-6-(trifluoromethyl)quinazolin-7-yl) benzo[d]thiazol-2-yl)carbamate (100 mg, 0.152 mmol) in ACN (15 mL) was added benzyl ((1S,4R)-7-azabicyclo [2.2.1]heptan-2-yl) (3-(dimethylamino)-3-oxopropyl) carbamate (145 mg, 0.381 mmol), benzotriazole-1-yl-oxytripyrrolidinophosphonium hexafluoro phosphate (118.6 mg, 0.228 mmol) and DIPEA (118 mg, 0.912 mmol) at room temperature, and the mixture was stirred at room temperature for overnight. Solvent was concentrated and the residue was purified by chromatography column on silica (eluting with DCM/MeOH=25/1) to give the title product (120 mg). MS (ESI, m/e) [M+H]⁺ 983.

### Step 5: tert-butyl (4-(4-((1S,4R)-2-((3-(dimethylamino)-3-oxopropyl) amino)-7-azabicyclo [2.2.1] heptan-7-yl)-8-fluoro-2-(((2R,7aS)-2-fluorotetrahydro-1H-pyrrolizin-7a(5H)-yl) methoxy)-6-(trifluoromethyl) quinazolin-7-yl)-7-fluorobenzo[d] thiazol-2-yl) carbamate

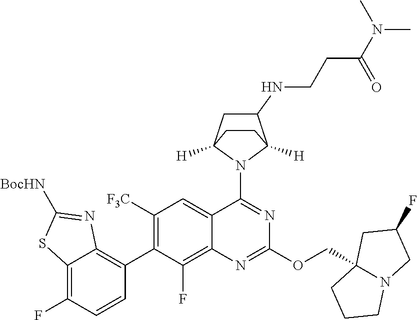 [SMILES: CN(C)C(=O)CCNC1C[C@H]2CC[C@@H]1N2c1nc(OC[C@@]23CCCN2C[C@H](F)C3)nc2c(F)c(-c3ccc(F)c4sc(NC(=O)OC(C)(C)C)nc34)c(C(F)(F)F)cc12]

To a solution of benzyl ((1S,4R)-7-(7-(2-((tert-butoxycarbonyl) amino)-7-fluorobenzo[d]thiazol-4-yl)-8-fluoro-2-(((2R,7aS)-2-fluorotetrahydro-1H-pyrrolizin-7a(5H)-yl) methoxy)-6-(trifluoromethyl) quinazolin-4-yl)-7-azabicyclo [2.2.1] heptan-2-yl) (3-(dimethylamino)-3-oxopropyl) carbamate (120 mg, 0.12 mmol) in MeOH (20 mL) was added Pd/C (60 mg) at room temperature, and the mixture was stirred under hydrogen at room temperature for 3 h. The resulting mixture was concentrated to give a residue (100 mg). MS (ESI, m/e) [M+H]⁺ 849.

### Step 6: 3-(((1S,4R)-7-(7-(2-amino-7-fluorobenzo[d]thiazol-4-yl)-8-fluoro-2-(((2R,7aS)-2-fluorotetrahydro-1H-pyrrolizin-7a(5H)-yl) methoxy)-6-(trifluoromethyl) quinazolin-4-yl)-7-azabicyclo [2.2.1] heptan-2-yl) amino)-N,N-dimethylpropanamide

To a solution of tert-butyl (4-(4-((1S,4R)-2-((3-(dimethylamino)-3-oxo propyl) amino)-7-azabicyclo [2.2.1] heptan-7-yl)-8-fluoro-2-(((2R,7aS)-2-fluorotetrahydro-1H-pyrrolizin-7a(5H)-yl) methoxy)-6-(trifluoromethyl) quinazolin-7-yl)-7-fluorobenzo [d]thiazol-2-yl) carbamate (100 mg, 0.118 mmol) in DCM (6 mL) was added TFA (8 mL) at room temperature, and the mixture was stirred at room temperature for 1 h. The resulting mixture was concentrated and pH was adjusted to 7 with Na₂CO₃. The organic layer was separated and solvents were removed to give a residue which was further purified by Prep-HPLC to give title product (18.93 mg). ¹H NMR (500 MHz, CD₃OD) δ 8.24 (s, 1H), 7.21-7.18 (m, 1H), 6.99-6.96 (m, 1H), 5.58-5.47 (m, 1H), 5.16-5.12 (m, 1H), 4.99-5.01 (m, 1H), 4.64 (m, 2H), 3.94-3.71 (m, 4H), 3.41-3.36 (m, 1H), 3.18-3.11 (m, 2H), 3.07 (s, 3H), 2.97 (s, 3H), 2.79-2.78 (m, 2H), 2.68-2.49 (s, 3H), 2.41-2.37 (m, 1H), 2.30-2.06 (m, 6H), 1.84-1.83 (m, 1H), 1.56-1.51 (m, 1H). MS (ESI, m/e) [M+H]⁺ 749.1.

### Example 100: 6-(4-(3,8-diazabicyclo[3.2.1]octan-3-yl)-6-(difluoromethyl)-8-fluoro-2-(((2R,7aS)-2-fluorotetrahydro-1H-pyrrolizin-7a(5H)-yl)methoxy)quinazolin-7-yl)-4-methyl-5-(trifluoromethyl)pyridin-2-amine

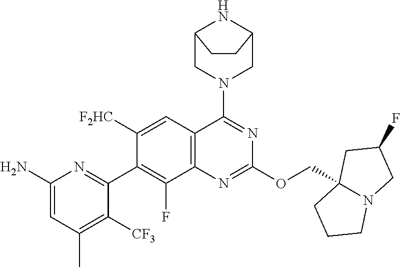 [SMILES: Cc1cc(N)nc(-c2c(C(F)F)cc3c(N4CC5CCC(C4)N5)nc(OC[C@@]45CCCN4C[C@H](F)C5)nc3c2F)c1C(F)(F)F]

Synthetic Route

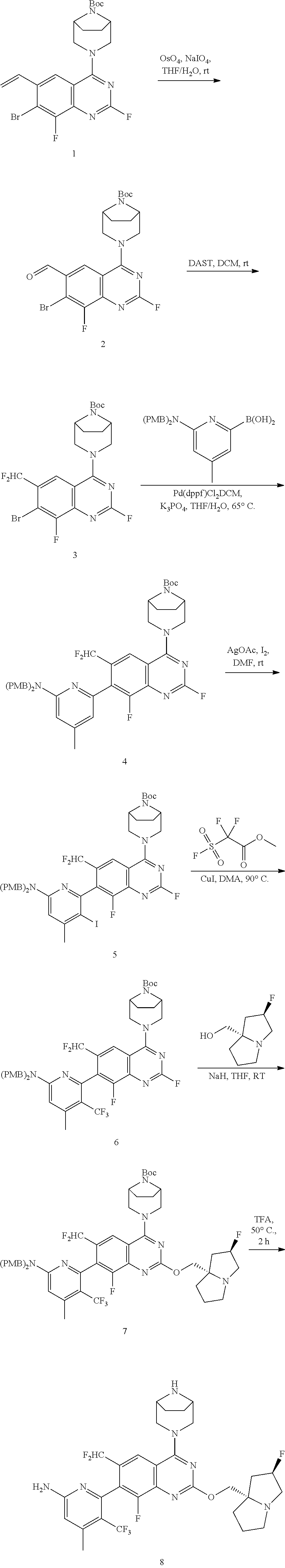

### Step 1: tert-butyl 3-(7-bromo-2,8-difluoro-6-formylquinazolin-4-yl)-3,8-diazabicyclo[3.2.1]octane-8-carboxylate

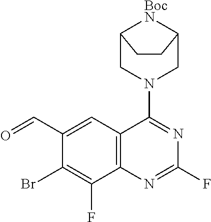 [SMILES: CC(C)(C)OC(=O)N1C2CCC1CN(c1nc(F)nc3c(F)c(Br)c(C=O)cc13)C2]

A solution of tert-butyl 3-(7-bromo-6-ethenyl-2,8-difluoroquinazolin-4-yl)-3,8-diazabicyclo[3.2.1]octane-8-carboxylate (1.01 g, 2.09 mmol) in THF (20 mL) and H₂O (2 mL) was added NaIO₄ (1.35 g, 6.29 mmol) and OsO₄ (21.34 mg, 0.08 mmol) in portions at 0° C. The resulting mixture was stirred for 2 h at rt. The resulting mixture was extracted with EtOAc. The combined organic layers were washed with brine, dried over anhydrous Na₂SO₄. After filtration, the filtrate was concentrated under reduced pressure. The residue was purified by Prep-TLC (CH₂Cl₂/MeOH=20:1) to give the titled product (675 mg, 66% yield). MS (ESI, m/e) [M+H]⁺ 483.05.

### Step 2: tert-butyl 3-(7-bromo-6-(difluoromethyl)-2,8-difluoroquinazolin-4-yl)-3,8-diazabicyclo[3.2.1]octane-8-carboxylate

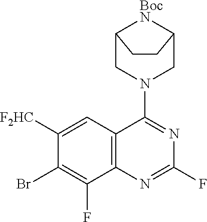 [SMILES: CC(C)(C)OC(=O)N1C2CCC1CN(c1nc(F)nc3c(F)c(Br)c(C(F)F)cc13)C2]

To a Teflon test tube was added tert-butyl 3-(7-bromo-2,8-difluoro-6-formyl quinazolin-4-yl)-3,8-diazabicyclo[3.2.1]octane-8-carboxylate (675 mg, 1.39 mmol) in DCM (20 mL). To the above mixture was added DAST (450.24 mg, 2.79 mmol) dropwise portions at 0° C. The resulting mixture was stirred for 3 h at rt. The reaction was quenched with NaHCO₃ (aq) at 0° C. The resulting mixture was extracted with CH₂Cl₂. The combined organic layers were washed with brine, dried over anhydrous Na₂SO₄. After filtration, the filtrate was concentrated under reduced pressure. The residue was purified by Prep-TLC (CH₂Cl₂/MeOH=20:1) to give the title product (300 mg, 42% yield). MS (ESI, m/e) [M+H]⁺ 505.05.

### Step 3: tert-butyl 3-(7-(6-(bis(4-methoxybenzyl)amino)-4-methylpyridin-2-yl)-6-(difluoromethyl)-2,8-difluoroquinazolin-4-yl)-3,8-diazabicyclo[3.2.1]octane-8-carboxylate

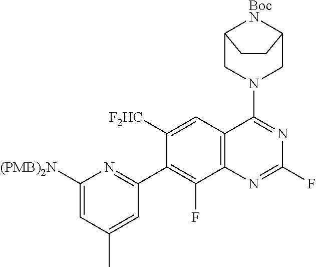

To a stirred solution of tert-butyl 3-(7-bromo-6-(difluoromethyl)-2,8-difluoro quinazolin-4-yl)-3,8-diazabicyclo[3.2.1]octane-8-carboxylate (300 mg, 0.59 mmol) and 6-(bis((4-methoxyphenyl)methyl)amino)-4-methylpyridin-2-ylboronic acid (582.20 mg, 1.48 mmol) in THF (8 mL) and H₂O (1.5 mL) was added K₃PO₄ (252.04 mg, 1.18 mmol) and Pd(dppf)Cl₂·CH₂Cl₂ (48.36 mg, 0.05 mmol) in portions at rt. The resulting mixture was stirred for 2 h at 65° C. The resulting mixture was extracted with EtOAc. The combined organic layers were washed with brine, dried over anhydrous Na₂SO₄. After filtration, the filtrate was concentrated under reduced pressure. The residue was purified by Prep-TLC (PE/EA=3:1) to give the title product (340 mg, 74% yield). MS (ESI, m/e) [M+H]⁺ 773.35.

### Step 4: tert-butyl 3-(7-(6-(bis(4-methoxybenzyl)amino)-3-iodo-4-methylpyridin-2-yl)-6-(difluoromethyl)-2,8-difluoroquinazolin-4-yl)-3,8-diazabicyclo[3.2.1]octane-8-carboxylate

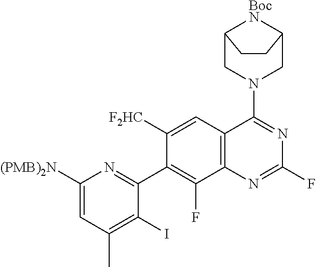

A solution of tert-butyl 3-(7-(6-(bis((4-methoxyphenyl)methyl]amino)-4-methylpyridin-2-yl)-6-(difluoromethyl)-2,8-difluoroquinazolin-4-yl)-3,8-diazabicyclo[3.2.1] octane-8-carboxylate (320 mg, 0.41 mmol), silver acetate (172.78 mg, 1.03 mmol) and I₂ (315.27 mg, 1.24 mmol) in DMF (5 mL) was stirred for 2 h at rt. The resulting mixture was extracted with EtOAc. The combined organic layers were washed with Na₂S₂O₃ (aq) and brine, dried over anhydrous Na₂SO₄. After filtration, the filtrate was concentrated under reduced pressure. The residue was purified by Prep-TLC (PE/EA=3:1) to give the title product (320 mg, 85% yield). MS (ESI, m/e) [M+H]⁺ 899.23.

### Step 5: tert-butyl 3-(7-(6-(bis(4-methoxybenzyl)amino)-4-methyl-3-(trifluoro methyl)pyridin-2-yl)-6-(difluoromethyl)-2,8-difluoroquinazolin-4-yl)-3,8-diazabicyclo[3.2.1] octane-8-carboxylate

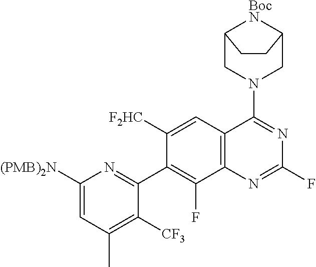

A solution of tert-butyl 3-(7-(6-(bis((4-methoxyphenyl)methyl)amino)-3-iodo-4-methylpyridin-2-yl)-6-(difluoromethyl)-2,8-difluoroquinazolin-4-yl)-3,8-diazabicyclo[3.2.1] octane-8-carboxylate (310 mg, 0.34 mmol) and CuI (262.77 mg, 1.38 mmol) in DMA (6 mL) was stirred for 15 min at rt under nitrogen atmosphere. To the above mixture was added methyl 2,2-difluoro-2-sulfoacetate (993.96 mg, 5.17 mmol) dropwise portions at rt. The resulting mixture was stirred for 2 h at 90° C. under nitrogen atmosphere. The resulting mixture was extracted with EtOAc. The combined organic layers were washed with brine, dried over anhydrous Na₂SO₄. After filtration, the filtrate was concentrated under reduced pressure. The residue was purified by Prep-TLC (PE/EA=3:1) to give the title product (256 mg, 88% yield). MS (ESI, m/e) [M+H]⁺ 841.35.

### Step 5: tert-butyl 3-(7-(6-(bis(4-methoxybenzyl)amino)-4-methyl-3-(trifluoro methyl)pyridin-2-yl)-6-(difluoromethyl)-8-fluoro-2-(((2R,7aS)-2-fluorotetrahydro-1H-pyrrolizin-7a(5H)-yl)methoxy)quinazolin-4-yl)-3,8-diazabicyclo[32._l]octane-8-carboxylate

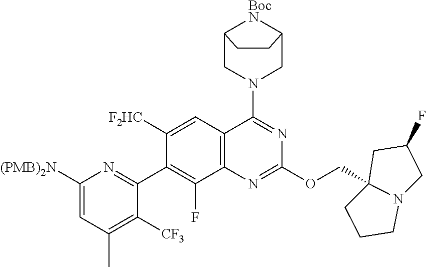

To a stirred solution of tert-butyl 3-(7-(6-(bis((4-methoxyphenyl)methyl)amino)-4-methyl-3-(trifluoromethyl)pyridin-2-yl)-6-(difluoromethyl)-2,8-difluoroquinazolin-4-yl)-3,8-diazabicyclo[3.2.1] octane-8-carboxylate (256 mg, 0.30 mmol) and ((2R,7aS)-2-fluoro-hexa hydropyrrolizin-7a-yl)methanol (53.32 mg, 0.33 mmol) in THF (6 mL) was added NaH (18.27 mg, 0.45 mmol, 60% in oil) in portions at 0° C. The resulting mixture was stirred for 2 h at rt. The reaction was quenched with Water/Ice at 0° C. The resulting mixture was extracted with EtOAc. The combined organic layers were washed with brine, dried over anhydrous Na₂SO₄. After filtration, the filtrate was concentrated under reduced pressure. The residue was purified by Prep-TLC (EA %=100%) to give the title product (230 mg, 77% yield). MS (ESI, m/e) [M+H]⁺ 980.45.

### Step 5: 6-(4-(3,8-diazabicyclo[3.2.1]octan-3-yl)-6-(difluoromethyl)-8-fluoro-2-(((2R,7aS)-2-fluorotetrahydro-1H-pyrrolizin-7a(5H)-yl)methoxy)quinazolin-7-yl)-4-methyl-5-(trifluoromethyl)pyridin-2-amine

A solution of tert-butyl 3-(2-(((2R,7aS)-2-fluoro-hexahydropyrrolizin-7a-yl) methoxy)-7-(6-(bis((4-methoxy phenyl)methyl)amino)-4-methyl-3-(trifluoromethyl)pyridin-2-yl)-6-(difluoromethyl)-8-fluoro quinazolin-4-yl)-3,8-diazabicyclo[3.2.1]octane-8-carboxylate (230 mg, 0.23 mmol) in TFA (2 mL) was stirred for 2 h at 50° C. The resulting mixture was concentrated under reduced pressure. The residue was purified by Prep-HPLC (Mobile Phase A: Water (0.1% FA), Mobile Phase B: ACN; Flow rate: 60 mL/min; Gradient: 5% B to 30% B in 8 min, 30% B) to give the titled product (10.3 mg, 6% yield). ¹H NMR (400 MHz, DMSO-d₆) δ 8.14 (d, J=4.4 Hz, 1H), 7.00-6.70 (m, 3H), 6.49 (s, 1H), 5.30 (d, J=54.1 Hz, 1H), 4.40 (dd, J=29.2, 13.4 Hz, 2H), 4.20-4.00 (m, 1H), 3.80-3.62 (m, 1H), 3.14 (m, 3H), 3.07 (m, 1H), 2.86 (m, 1H), 2.45 (m, 1H), 2.36 (d, J=2.0 Hz, 3H), 2.15 (m, 1H), 2.08 (m, 1H), 2.03 (m, 1H), 1.89 (m, 8H), 1.80 (m, 2H). MS (ESI, m/e) [M+H]⁺ 640.20.

### Example 101: 4-(4-(3,8-diazabicyclo[3.2.1]octan-8-yl)-8-chloro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

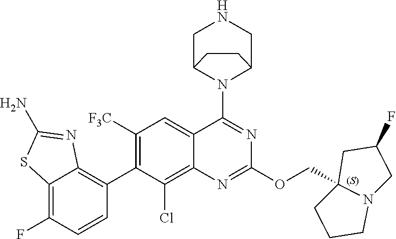

Synthetic Route

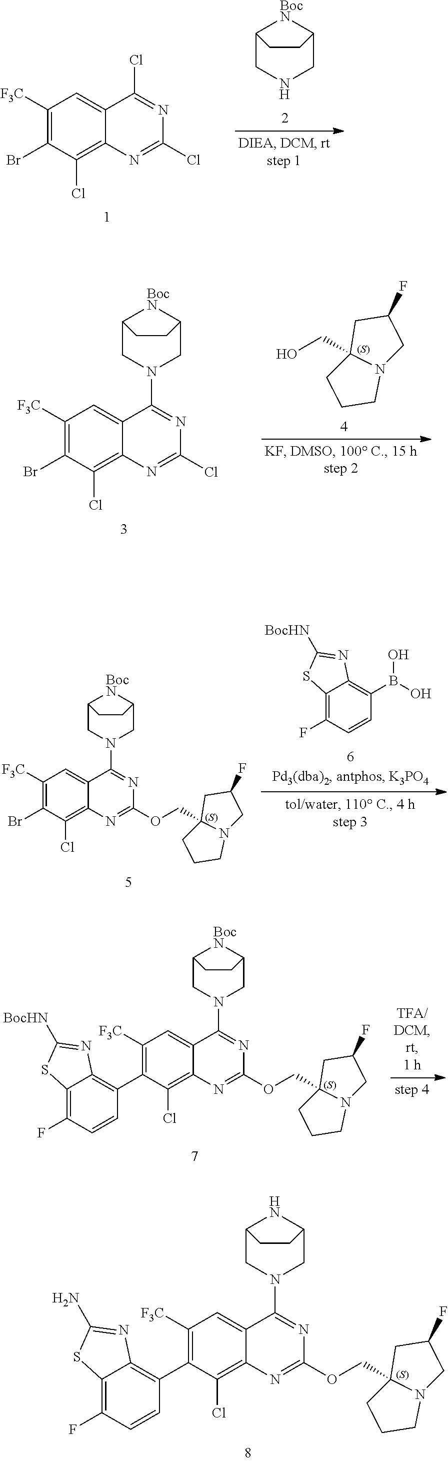

### Step 1: tert-butyl 8-(7-bromo-2,8-dichloro-6-(trifluoromethyl)quinazolin-4-yl)-3,8-diazabicyclo[3.2.1]octane-3-carboxylate

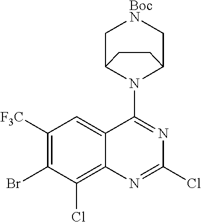 [SMILES: CC(C)(C)OC(=O)N1CC2CCC(C1)N2c1nc(Cl)nc2c(Cl)c(Br)c(C(F)(F)F)cc12]

To a solution of 7-bromo-2,4,8-trichloro-6-(trifluoromethyl)quinazoline (2800 mg, 7.4 mmol) and DIEA (2870 mg, 22.2 mmol) in DCM (50 mL) was added tert-butyl 3,8-diazabicyclo[3.2.1]octane-3-carboxylate (1570 mg, 7.4 mmol) at room temperature. The resulting mixture was stirred at room temperature for 1 hr. After completion, the reaction mixture was concentrated and purified by silica gel (PE:EA=4:1) to give the title product (3.1 g, 75.4%). MS (ESI, m/e) [M−55]⁺555.

### Step 2: tert-butyl 8-(7-bromo-8-chloro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1] octane-3-carboxylate

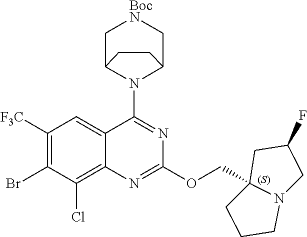

To a solution of tert-butyl 8-(7-bromo-2,8-dichloro-6-(trifluoromethyl)quinazolin-4-yl)-3,8-diazabicyclo[3.2.1]octane-3-carboxylate (1.55 g, 2.79 mmol) and ((2R,7aS)-2-fluoro tetrahydro-1H-pyrrolizin-7a(5H)-yl)methanol (1330 mg, 8.37 mmol) in DMSO (50 mL) was added KF (809 mg, 14.0 mmol) at 25° C. The resulting mixture was heated at 100° C. for 15 hrs. After completion, the mixture was decanted into ice-water and filtered. The solid was washed with water to give the title product (560 mg, 29.7%). MS (ESI, m/e) [M−55]⁺678, 680.

### Step 3: tert-butyl 8-(7-(2-((tert-butoxycarbonyl)amino)-7-fluorobenzo[d]thiazol-4-yl)-8-chloro-2-(((2R,7aS)-2-fluorotetrahydro-1H-pyrrolizin-7a(5H)-yl)methoxy)-6-(trifluoro methyl)quinazolin-4-yl)-3,8-diazabicyclo[3.2.1]octane-3-carboxylate

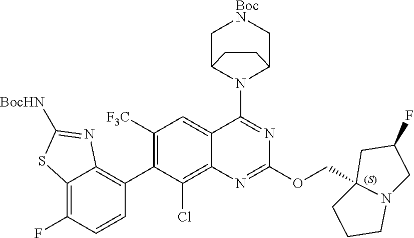

The mixture of tert-butyl 8-(7-bromo-8-chloro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1] octane-3-carboxylate (226 mg, 0.333 mmol), (2-((tert-butoxycarbonyl)amino)-7-fluorobenzo[d] thiazol-4-yl)boronic acid (312 mg, 1.0 mmol) and Pd₂(dba)₃ (31 mg, 0.0333 mmol), (R)-Antphos (25 mg, 0.0666 mmol) and K₃PO₄ (212 mg, 1.0 mmol) in dioxane (10 mL) and H₂O (2 mL) was stirred at 100° C. for 5 hrs under N₂ atmosphere. After completion, the reaction mixture was purified by Prep-TLC (PE:EA=1:2) to give the title product (120 mg, 41.7%). MS (ESI, m/e) [M+H]⁺ 866.

### Step 4: 4-(4-(3,8-diazabicyclo[3.2.1]octan-8-yl)-8-chloro-2-(((2R,7aS)-2-fluoro tetrahydro-1H-pyrrolizin-7a(5H)-yl)methoxy)-6-(trifluoromethyl)quinazolin-7-yl)-7-fluorobenzo [d]thiazol-2-amine

To a solution of tert-butyl 8-(7-(2-((tert-butoxycarbonyl)amino)-7-fluorobenzo[d]thiazol-4-yl)-8-chloro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-3-carboxylate (120 mg, 0.139 mmol) in DCM (5 mL) was added TFA (2.5 mL) at room temperature. The resulting mixture was stirred at room temperature for 1 hr. After completion, the reaction mixture was concentrated to give the residue. The residue was purified by Prep-HPLC to give the title product (40.8 mg, 44.0%). ¹H NMR (500 MHz, CD₃OD) δ 8.33 (s, 1H), 7.20-7.04 (m, 1H), 7.00-6.97 (m, 1H), 5.55-5.45 (m, 1H), 5.20-5.01 (m, 2H), 4.70-4.59 (m, 2H), 3.92-3.73 (m, 3H), 3.51-3.40 (m, 3H), 3.22-3.16 (m, 2H), 2.66-2.47 (m, 2H), 2.44-2.25 (m, 3H), 2.23-1.99 (m, 5H). MS (ESI, m/e) [M+H]⁺ 665.9.

### Example 102: 6-(4-(3,8-diazabicyclo[3.2.1]octan-8-yl)-6-chloro-8-fluoro-2-(((2R,7aS)-2-fluorotetrahydro-1H-pyrrolizin-7a(5H)-yl)methoxy)quinazolin-7-yl)-4-chloro-5-(trifluoromethyl)pyridin-2-amine

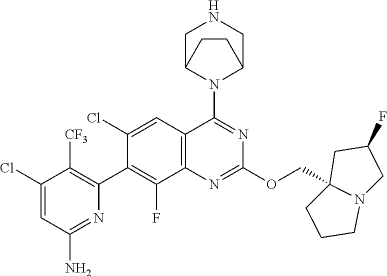 [SMILES: Nc1cc(Cl)c(C(F)(F)F)c(-c2c(Cl)cc3c(N4C5CCC4CNC5)nc(OC[C@@]45CCCN4C[C@H](F)C5)nc3c2F)n1]

Synthetic Route

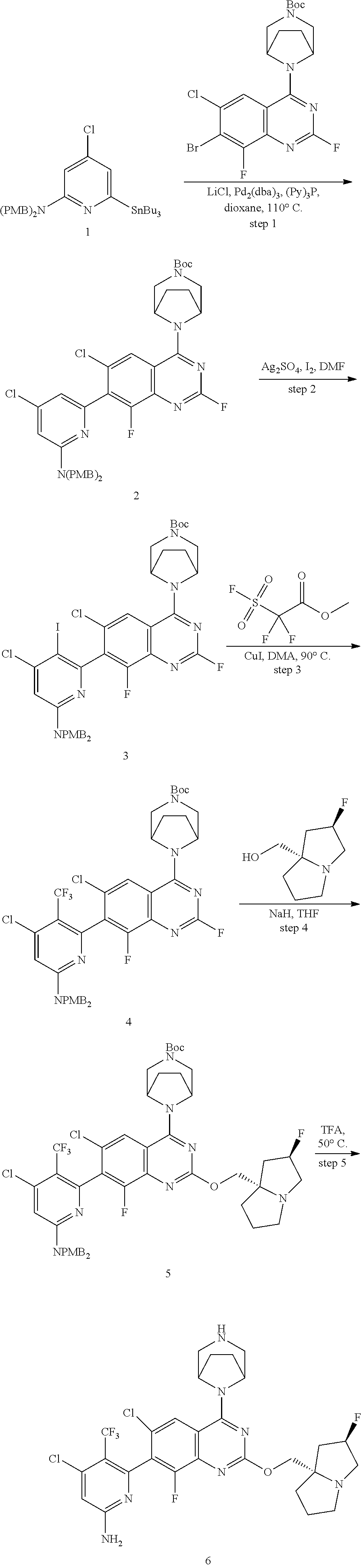

### Step 1: tert-butyl 8-(7-(6-(bis(4-methoxybenzyl)amino)-4-chloropyridin-2-yl)-6-chloro-2,8-difluoroquinazolin-4-yl)-3,8-diazabicyclo[3.2.1]octane-3-carboxylate

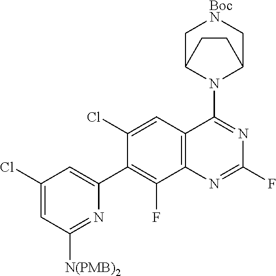

Into a 50-mL round-bottom flask were placed tert-butyl 8-(7-bromo-6-chloro-2,8-difluoroquinazolin-4-yl)-3,8-diaza bicyclo[3.2.1]octane-3-carboxylate (500 mg, 0.759 mmol, 1.00 equiv), 1,4-dioxane (5 mL), 4-chloro-N, N-bis(4-methoxybenzyl)-6-(tributylstannyl)pyridin-2-amine (400 mg, 0.819 mmol, 1.1 equiv), Pd₂(dba)₃ (140 mg, 0.153 mmol, 0.2 equiv), Cy₃P (90 mg, 0.321 mmol, 0.4 equiv), LiCl (60 mg, 1.428 mmol, 2.0 equiv). The resulting solution was stirred for one night at 110° C. in an oil bath. The resulting solution was diluted with 100 mL of water. The resulting solution was extracted with 100 mL of ethyl acetate. The resulting mixture was washed with 3×800 mL of brine. The resulting mixture was concentrated under vacuum. The crude product was purified by combi-Flash with the following conditions: Column, silica gel; mobile phase, PE:EA=100:0 increasing to PE:EA=90:10 within 1 hr; Detector, 254/280 nm. This resulted in 294 mg (49.9% yield) of the title product. MS (ESI, m/e) [M+H]⁺ 777.17.

### Step 2: tert-butyl 8-(7-(6-(bis(4-methoxybenzyl)amino)-4-chloro-3-iodopyridin-2-yl)-6-chloro-2,8-difluoroquinazolin-4-yl)-3,8-diazabicyclo [3.2.1]octane-3-carboxylate

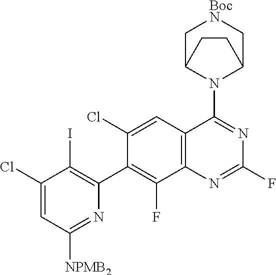

Into a 50-mL round-bottom flask, were placed tert-butyl 8-(7-(6-(bis(4-methoxy benzyl)amino)-4-chloropyridin-2-yl)-6-chloro-2,8-difluoroquinazolin-4-yl)-3,8-diazabicyclo [3.2.1]octane-3-carboxylate (294 mg, 0.378 mmol, 1.00 equiv), DMF (3 mL). This was followed by the addition of Ag₂SO₄ (153 mg, 0.753 mmol, 2.0 equiv) in portions at rt in 10 min. The resulting solution was stirred for 5 min at room temperature. To this was added I₂ (115 mg, 0.452 mmol, 1.2 equiv) at rt. The resulting solution was allowed to react with stirring for 2 h at room temperature. The resulting solution was diluted with 100 ml of EA. The resulting mixture was filtered, the filter cake was washed with EA. The resulting solution was extracted with EA. The resulting mixture was washed with 3×50 mL of brine. The resulting mixture was concentrated under vacuum. The crude product was purified by combi-Flash with the following conditions: Column, silica gel; mobile phase, PE:EA=100:0 increasing to PE:EA=75:25 within 1 h; Detector, 254/280 nm. This resulted in 158 mg (46.33% yield) of the title product. MS (ESI, m/e) [M+H]⁺ 903.20.

### Step 3: tert-butyl 8-(7-(6-(bis(4-methoxybenzyl)amino)-4-chloro-3-(trifluoro methyl)pyridin-2-yl)-6-chloro-2,8-difluoroquinazolin-4-yl)-3,8-diazabicyclo[3.2.1]octane-3-carboxylate

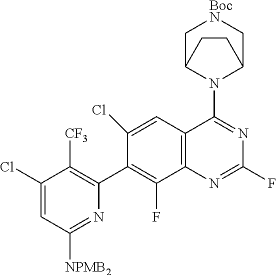

Into a 50-mL round-bottom flask were placed tert-butyl 8-(7-(6-(bis(4-methoxybenzyl)amino)-4-chloro-3-iodopyridin-2-yl)-6-chloro-2,8-difluoroquinazolin-4-yl)-3,8-diazabicyclo[3.2.1]octane-3-carboxylate (158 mg, 0.175 mmol, 1.00 equiv), DMA (3 mL), CuI (133 mg, 0.700 mmol, 4 equiv), methyl 2,2-difluoro-2-sulfoacetate (672 mg, 3.500 mmol, 20 equiv). The resulting solution was stirred 2 h at 90° C. in an oil bath. The resulting solution was diluted with 50 mL of EA. The resulting mixture was filtered, the filter cake was washed with EA. The resulting solution was extracted with EA. The resulting mixture was washed with 3×50 mL of brine. The resulting mixture was concentrated under vacuum. The crude product was purified by combi-Flash with the following conditions: Column, silica gel; mobile phase, PE:EA=100:0 increasing to PE:EA=65:35 within 1 h; Detector, 254/280 nm. This resulted in 80 mg (54.42% yield) of the title product. MS (ESI, m/e) [M+H]⁺ 845.10.

### Step 4: tert-butyl 8-(7-(6-(bis(4-methoxybenzyl)amino)-4-chloro-3-(trifluoro methyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-3-carboxylate

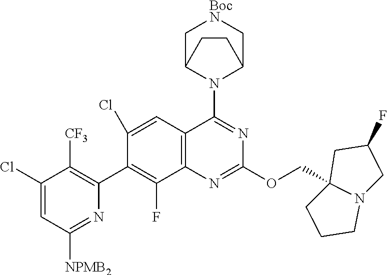

To a stirred solution of tert-butyl 8-(7-(6-(bis(4-methoxybenzyl)amino)-4-chloro-3-(trifluoromethyl)pyridin-2-yl)-6-chloro-2,8-difluoroquinazolin-4-yl)-3,8-diazabicyclo[3.2.1] octane-3-carboxylate (80 mg, 0.094 mmol, 1.00 equiv) and ((2R,7aS)-2-fluorotetrahydro-1H-pyrrolizin-7a(5H)-yl)methanol (18 mg, 0.113 mmol, 1.2 equiv) in THF (2 mL) were added NaH (60% in oil, 8 mg, 0.2 mmol) in portions at 0° C. under N₂ atmosphere. The resulting solution was stirred for 2 h at rt. The reaction was quenched with water at 0° C. The resulting solution was extracted with EA. The resulting mixture was washed with 3×20 mL of brine. The resulting mixture was concentrated under vacuum. The residue was purified by Prep-TLC (PE/EA=1:1) to afford the title product (31 mg, 54.4% yield). MS (ESI, m/e) [M+H]⁺ 984.40.

### Step 5: 6-(4-(3,8-diazabicyclo[3.2.1]octan-8-yl)-6-chloro-8-fluoro-2-(((2R,7aS)-2-fluorotetrahydro-1H-pyrrolizin-7a(5H)-yl)methoxy)quinazolin-7-yl)-4-chloro-5-(trifluoromethyl) pyridin-2-amine

The resulting mixture of tert-butyl 8-(7-(6-(bis(4-methoxybenzyl)amino)-4-chloro-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-3-carboxylate (31 mg, 0.031 mmol, 1.0 equiv) in TFA (1 ml) was stirred for 5 h at 50° C. The resulting mixture was concentrated under vacuum. The residue was dissolved in EA. The organic layer was basified by Na₂CO₃ (aq.). The resulting mixture was stirred for 30 min at rt. The resulting mixture was extracted with EA. The combined organic layers were washed with water, dried over anhydrous Na₂SO₄. After filtration, the filtrate was concentrated under reduced pressure. The crude product (10 mg) was purified by Prep-HPLC to give the titled product (7.20 mg, FA salt, 36% yield). ¹H NMR (400 MHz, CD₃OD) δ 8.48 (s, 1H), 7.93 (d, J=1.6 Hz, 1H), 6.84 (s, 1H), 5.55-5.42 (m, 1H), 5.05 (s, 2H), 4.60-4.46 (m, 2H), 3.82 (dd, J=13.9, 2.9 Hz, 1H), 3.76-3.59 (m, 3H), 3.45 (s, 2H), 3.44-3.32 (m, 2H), 3.15 (d, J=12.8 Hz, 2H), 2.40-2.00 (m, 8H). MS (ESI, m/e) [M+H]⁺ 644.20.

### Example 103: 4-(4-(3,8-diazabicyclo[3.2.1]octan-3-yl)-8-fluoro-2-(((2R,4R)-4-fluoro-1,2-dimethylpyrrolidin-2-yl)methoxy)-6-(trifluoromethyl)quinazolin-7-yl)-7-fluorobenzo [d]thiazol-2-amine

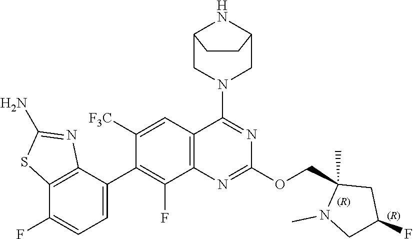

Synthetic Route

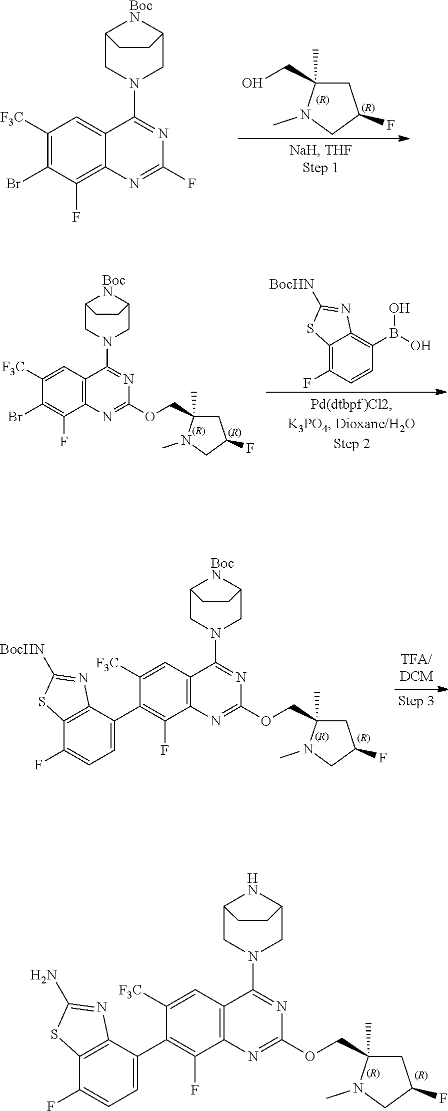

### Step 1: tert-butyl 3-(7-bromo-8-fluoro-2-(((2R,4R)-4-fluoro-1,2-dimethyl pyrrolidin-2-yl)methoxy)-6-(trifluoromethyl)quinazolin-4-yl)-3,8-diazabicyclo[3.2.1]octane-8-carboxylate

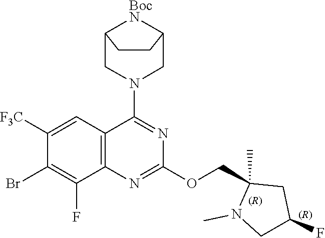

To a solution of ((2R,4R)-4-fluoro-1,2-dimethylpyrrolidin-2-yl)methanol (176.5 mg, 1.2 mmol) in THF (5 mL) was added sodium hydride (48.0 mg, 1.2 mmol) at room temperature. The resulting mixture was stirred at room temperature for 1 hrs. Then tert-butyl 3-(7-bromo-2,8-difluoro-6-(trifluoromethyl)quinazolin-4-yl)-3,8-diazabicyclo [3.2.1]octane-8-carboxylate (522.1 mg, 1.0 mmol) was added to the reaction mixture and stirred at room temperature for 1 hrs. After completion. The reaction mixture was diluted with EA (50 mL), washed with brine (15 mL×3). The organic layer was dried over anhydrous Na₂SO₄, filtered and the filtrate was concentrated to give the residue. The residue was purified by Prep-TLC (PE:EA=3:1) to give the title product (250 mg, 0.39 mmol). MS (ESI, m/e) [M+H]⁺ 650.2, 652.2.

### Step 2: tert-butyl 3-(7-(2-((tert-butoxycarbonyl)amino)-7-fluorobenzo[d]thiazol-4-yl)-8-fluoro-2-(((2R,4R)-4-fluoro-1,2-dimethylpyrrolidin-2-yl)methoxy)-6-(trifluoromethyl) quinazolin-4-yl)-3,8-diazabicyclo[3.2.1]octane-8-carboxylate

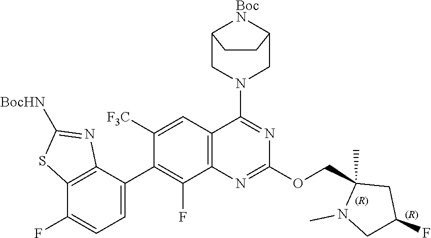

To a solution of tert-butyl 3-(7-bromo-8-fluoro-2-(((2R,4R)-4-fluoro-1,2-dimethyl pyrrolidin-2-yl)methoxy)-6-(trifluoromethyl)quinazolin-4-yl)-3,8-diazabicyclo [3.2.1] octane-8-carboxylate (150 mg, 0.23 mmol), (2-amino-7-fluorobenzo[d]thiazol-4-yl) boronic acid (93.6 mg, 0.3 mmol) and Pd(dtbpf)Cl₂ (12.9 mg, 0.02 mmol) in dioxane (5 mL) and H₂O (1 mL) was added K₃PO₄ (127.2 mg, 0.60 mmol) at room temperature, The resulting mixture was stirred at 100° C. for 16 hrs. After completion. The reaction mixture was diluted with EA (30 mL), washed with saturated brine (250 mL×3). The organic layer was dried over anhydrous Na₂SO₄, filtered and the filtrate was concentrated to give the residue. The residue was purified by Prep-TLC (pure EA) to give the title product (150 mg, 0.18 mmol). MS (ESI, m/e) [M+1]⁺838.3.

### Step 3: 4-(4-(3,8-diazabicyclo[3.2.1]octan-3-yl)-8-fluoro-2-(((2R,4R)-4-fluoro-1,2-dimethylpyrrolidin-2-yl)methoxy)-6-(trifluoromethyl)quinazolin-7-yl)-7-fluorobenzo[d] thiazol-2-amine

To a solution of tert-butyl 3-(7-(2-((tert-butoxycarbonyl)amino)-7-fluorobenzo [d]thiazol-4-yl)-8-fluoro-2-(((2R,4R)-4-fluoro-1,2-dimethylpyrrolidin-2-yl)methoxy)-6-(tri fluoromethyl)quinazolin-4-yl)-3,8-diazabicyclo[3.2.1]octane-8-carboxylate (150 mg, 0.18 mmol) in DCM (5 mL) was added TFA (5 mL) at room temperature. The resulting mixture was stirred at room temperature for 3 hrs. After completion. The reaction mixture was concentrated to give the residue. The residue was purified by High-HPLC to give the title product (47.56 mg). ¹H NMR (500 MHz, DMSO-d₆) δ 7.89 (s, 1H), 7.17 (s, 2H), 7.18-7.16 (m, 1H), 7.07-7.05 (m, 1H), 5.35-5.25 (m, 1H), 4.62-4.52 (m, 4H), 4.22-4.19 (m, 4H), 3.84-3.75 (m, 4H), 2.81 (s, 3H), 2.35-2.20 (m, 1H), 1.97-1.92 (m, 6H). MS (ESI, m/e) [M+1]⁺638.4.

### Example 104: 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(trifluoromethyl)quinazolin-7-yl)-2-amino-7-fluorobenzo[b]thiophene-3-carbonitrile

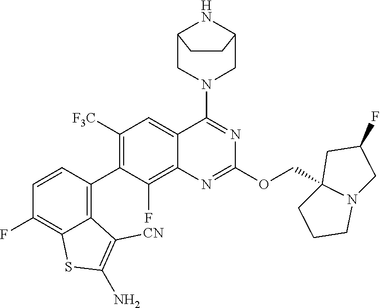 [SMILES: N#Cc1c(N)sc2c(F)ccc(-c3c(C(F)(F)F)cc4c(N5CC6CCC(C5)N6)nc(OC[C@@]56CCCN5C[C@H](F)C6)nc4c3F)c12]

Synthetic Route

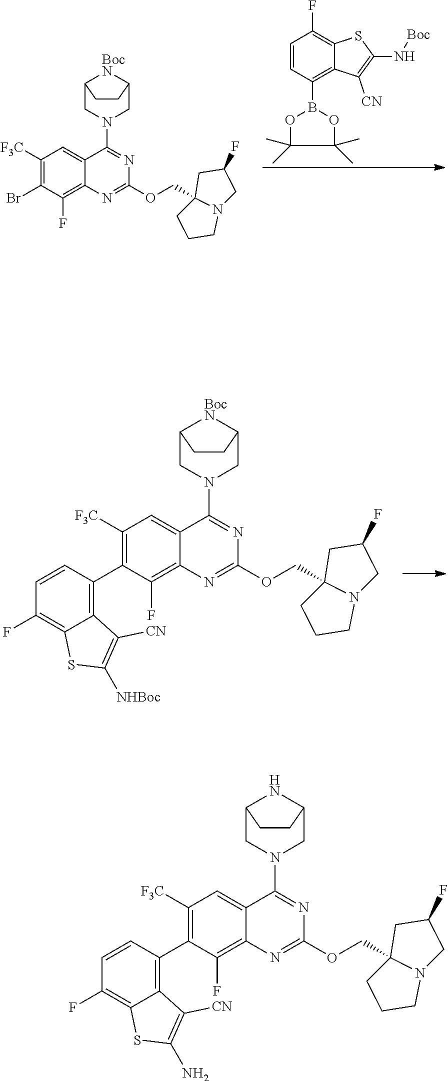

### Step 1: tert-butyl 3-(7-(2-((tert-butoxycarbonyl)amino)-3-cyano-7-fluorobenzo [b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3,8-diazabicyclo [3.2.1] octane-8-carboxylate

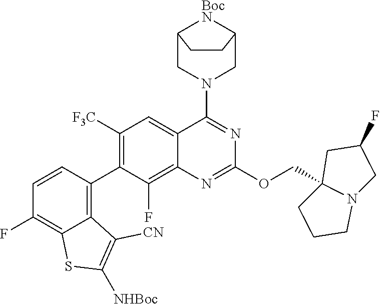 [SMILES: CC(C)(C)OC(=O)Nc1sc2c(F)ccc(-c3c(C(F)(F)F)cc4c(N5CC6CCC(C5)N6C(=O)OC(C)(C)C)nc(OC[C@@]56CCCN5C[C@H](F)C6)nc4c3F)c2c1C#N]

The mixture of tert-butyl 3-(7-bromo-8-fluoro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1] octane-8-carboxylate (132 mg, 0.2 mmol), tert-butyl (3-cyano-7-fluoro-4-(4,4,5,5-tetramethyl-1,3,2-dioxaborolan-2-yl)benzo[b]thiophen-2-yl)carbamate (167 mg, 0.4 mmol) and PdCl₂DPEphos (22 mg, 0.03 mmol), Cs₂CO₃ (196 mg, 0.6 mmol) in toluene (5 mL) was stirred at 100° C. for 15 hrs. After completion. The reaction mixture was purified by Prep-TLC (DCM:MeOH=10:1) to give the titled product (152 mg, crude product). MS (ESI, m/e) [M+H]⁺ 874.4.

### Step 2: 4-(4-(3,8-diazabicyclo[3.2.1]octan-3-yl)-8-fluoro-2-(((2R,7aS)-2-fluoro tetrahydro-1H-pyrrolizin-7a(5H)-yl)methoxy)-6-(trifluoromethyl)quinazolin-7-yl)-2-amino-7-fluorobenzo [b]thiophene-3-carbonitrile

To a solution of tert-butyl 3-(7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1]octane-8-carboxylate (152 mg, 0.174 mmol) in DCM (6 mL) was added TFA (3 mL) at room temperature. The resulting mixture was stirred at room temperature for 3 hrs. After completion, the reaction mixture was concentrated and the residue was purified by prep-HPLC to give the title product (80.0 mg, 23.8%). ¹H NMR (500 MHz, CD₃OD) δ 8.14 (s, 1H), 7.27-7.15 (m, 1H), 7.04-7.00 (m, 1H), 5.46-5.36 (m, 1H), 4.71-4.51 (m, 3H), 4.51-4.34 (m, 2H), 3.96-3.71 (m, 4H), 3.65-3.46 (m, 2H), 3.25-3.18 (m, 1H), 2.55-2.22 (m, 3H), 2.21-2.08 (m, 2H), 2.07-1.84 (m, 5H). MS (ESI, m/e) [M+H]⁺ 674.4.

### Example 105: 3-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-5-chloro-4-(trifluoromethyl)phenol

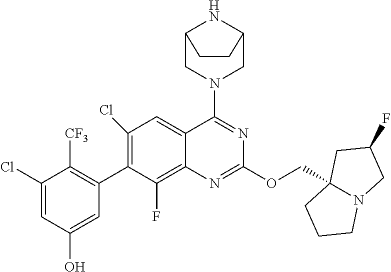 [SMILES: Oc1cc(Cl)c(C(F)(F)F)c(-c2c(Cl)cc3c(N4CC5CCC(C4)N5)nc(OC[C@@]45CCCN4C[C@H](F)C5)nc3c2F)c1]

Synthetic Route

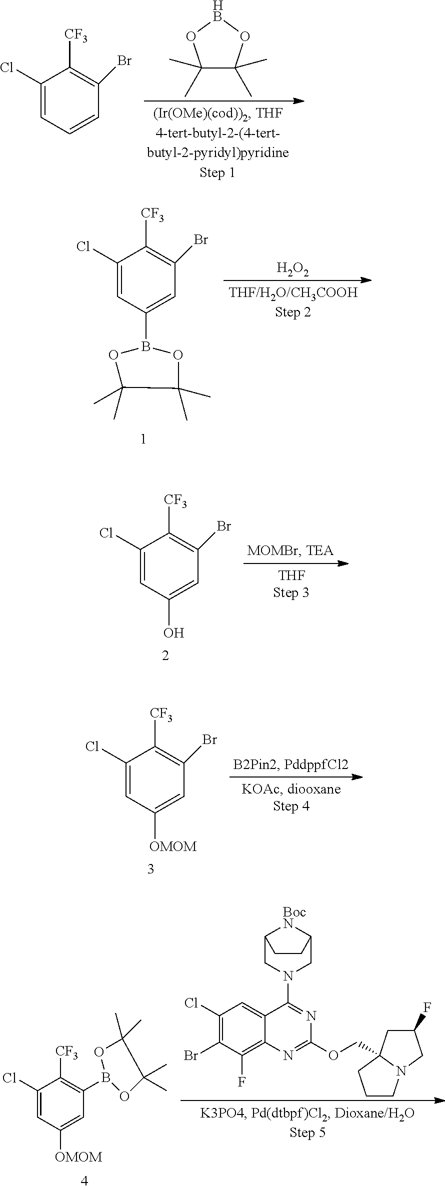

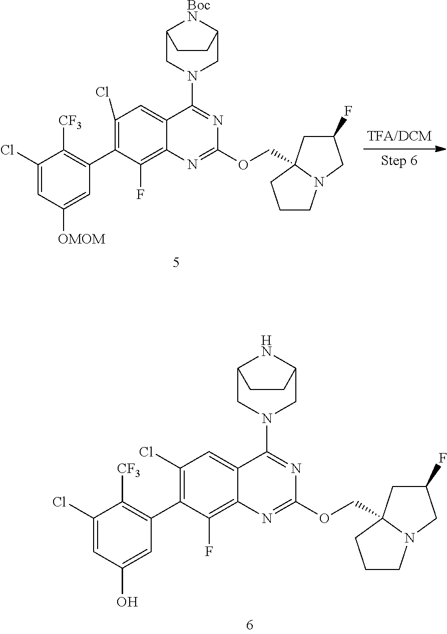

### Step 1: 2-(3-bromo-5-chloro-4-(trifluoromethyl)phenyl)-4,4,5,5-tetramethyl-1,3,2-dioxaborolane

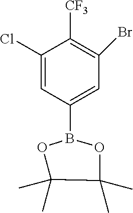 [SMILES: CC1(C)OB(c2cc(Cl)c(C(F)(F)F)c(Br)c2)OC1(C)C]

To a mixture of 1-bromo-3-chloro-2-(trifluoromethyl)benzene (2.58 g, 10 mmol) in THF (50 mL) was added 4,4,5,5-tetramethyl-1,3,2-dioxaborolane (3.84 g, 30 mmol), (Ir(OMe)(cod))₂ (664.2 mg, 1 mmol), 4-tert-butyl-2-(4-tert-butyl-2-pyridyl)pyridine (268.4 mg, 1 mmol) at room temperature. The resulting mixture was stirred at 60° C. for 2 hrs. After completion. The reaction mixture was concentrated to give the title compound (4.0 g, crude).

### Step 2: 3-bromo-5-chloro-4-(trifluoromethyl)phenol

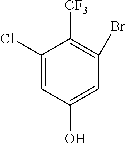 [SMILES: Oc1cc(Cl)c(C(F)(F)F)c(Br)c1]

To a mixture of 2-(3-bromo-5-chloro-4-(trifluoromethyl)phenyl)-4,4,5,5-tetramethyl-1,3,2-dioxaborolane (4 g, crude) in THF (30 mL) and H₂O (15 mL) were added CH₃COOH (30 mL) and 30% H₂O₂ (15 mL) at 0° C., The resulting mixture was stirred at 0° C. for 1 hrs. After completion, the reaction mixture was diluted with EA (150 mL), washed with saturated NaCl (250 mL×3). The organic layer was dried over anhydrous Na₂SO₄, filtered and the filtrate was concentrated to give the residue. The residue was purified by silica gel column chromatography, eluted with 0-30% EA in PE to give the title product (1.0 g, 0.37 mmol). MS (ESI, m/e) [M−1]⁺ 272.9.

### Step 3: 1-bromo-3-chloro-5-(methoxymethoxy)-2-(trifluoromethyl)benzene

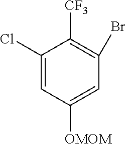

To a solution of 3-bromo-5-chloro-4-(trifluoromethyl)phenol (1 g, 3.65 mmol) and TEA (1.11 g, 10.95 mmol) in THF (25 mL) was added bromo(methoxy)methane (905.2 mg, 7.3 mmol) at room temperature. The resulting mixture was stirred at room temperature for 3 hrs. After completion. The reaction mixture was diluted with EA (150 mL), washed with brine (250 mL×3). The organic layer was dried over anhydrous Na₂SO₄, filtered and the filtrate was concentrated to give the residue. The residue was purified by silica gel column chromatography, eluted with 0-30% EA in PE to give the title product (1.0 g, 3.14 mmol).

### Step 4: 2-(3-chloro-5-(methoxymethoxy)-2-(trifluoromethyl)phenyl)-4,4,5,5-tetramethyl-1,3,2-dioxaborolane

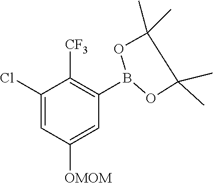

To a solution of 1-bromo-3-chloro-5-(methoxymethoxy)-2-(trifluoromethyl) benzene (317.9 mg, 1.0 mmol), PddppfCl₂ (73.1 mg, 0.1 mmol), 4,4,4′,4′,5,5,5′,5′-octamethyl-2,2′-bi(1,3,2-dioxaborolane) (762.6 mg, 3.0 mmol) in dioxane (5 mL) was added KOAc (294.0 mg, 3.0 mmol) at room temperature. The resulting mixture was stirred at 100° C. for 3 hrs. After completion, the reaction mixture was diluted with EA (150 mL), washed with brine (250 mL×3). The organic layer was dried over anhydrous Na₂SO₄, filtered and the filtrate was concentrated to give the residue. The residue was purified by silica gel column chromatography, eluted with 0-30% EA in PE to give the title product (200 mg, 0.54 mmol).

### Step 5: tert-butyl 3-(6-chloro-7-(3-chloro-5-(methoxymethoxy)-2-(trifluoro methyl)phenyl)-8-fluoro-2-(((2R,7aS)-2-fluorotetrahydro-1H-pyrrolizin-7a(5H)-yl)methoxy) quinazolin-4-yl)-3,8-diazabicyclo[3.2.1]octane-8-carboxylate

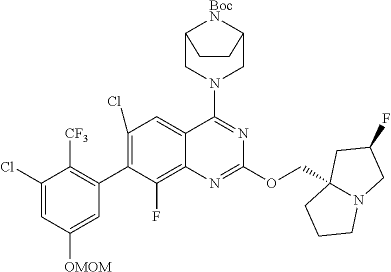

To a solution of tert-butyl 3-(7-bromo-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1] octane-8-carboxylate (62.7 mg, 0.1 mmol), 2-(3-chloro-5-(methoxymethoxy)-2-(trifluoro methyl)phenyl)-4,4,5,5-tetramethyl-1,3,2-dioxaborolane (73.22 mg, 0.2 mmol) and Pd(dtbpf)Cl₂ (6.5 mg, 0.01 mmol) in dioxane (5 mL) and H₂O (1 mL) was added K₃PO₄ (63.6 mg, 0.30 mmol) at room temperature, The resulting mixture was stirred at 100° C. for 16. After completion. The reaction mixture was diluted with EA (30 mL), washed with brine (250 mL×3). The organic layer was dried over anhydrous Na₂SO₄, filtered and the filtrate was concentrated to give the residue. The residue was purified by Prep-TLC (pure EA) to give the title product (50 mg, 0.06 mmol). MS (ESI, m/e) [M+1]⁺788.2.

### Step 6: 3-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-5-chloro-4-(trifluoromethyl) phenol

To a solution of tert-butyl 3-(6-chloro-7-(3-chloro-5-(methoxymethoxy)-2-(tri fluoromethyl)phenyl)-8-fluoro-2-(((2R,7aS)-2-fluorotetrahydro-1H-pyrrolizin-7a(5H)-yl) methoxy)quinazolin-4-yl)-3,8-diazabicyclo[3.2.1]octane-8-carboxylate (50 mg, 0.06 mmol) in DCM (5 mL) was added TFA (5 mL) at room temperature. The resulting mixture was stirred at room temperature for 3 hrs. After completion, the reaction mixture was concentrated to give the residue. The residue was purified by High-HPLC to give the title product (13.1 mg). ¹H NMR (500 MHz, DMSO-d₆) δ 7.89 (s, 1H), 7.21 (s, 1H), 6.68 (s, 1H), 5.33-5.22 (m, 1H), 4.40-4.30 (m, 2H), 4.00-4.38 (m, 2H), 3.66-3.62 (m, 2H), 3.35-3.30 (m, 2H), 3.14-2.97 (m, 3H), 2.83-2.81 (m, 1H), 2.06-2.00 (m, 3H), 1.86-1.77 (m, 7H). MS (ESI, m/e) [M+H]⁺ 644.4.

### Example 106: 4-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile

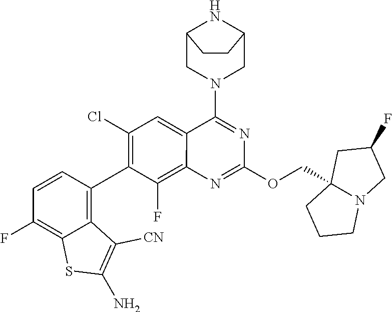 [SMILES: N#Cc1c(N)sc2c(F)ccc(-c3c(Cl)cc4c(N5CC6CCC(C5)N6)nc(OC[C@@]56CCCN5C[C@H](F)C6)nc4c3F)c12]

Synthetic Route

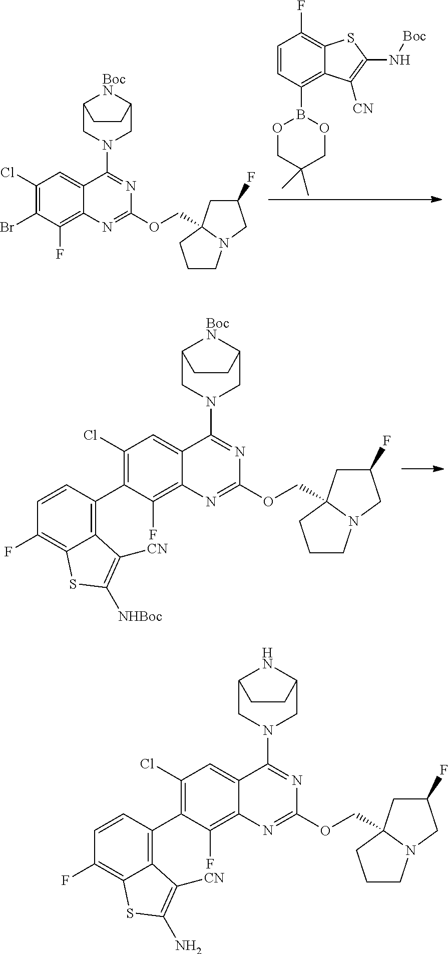

### Step 1: tert-butyl 3-(7-(2-((tert-butoxycarbonyl)amino)-3-cyano-7-fluorobenzo [b]thiophen-4-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

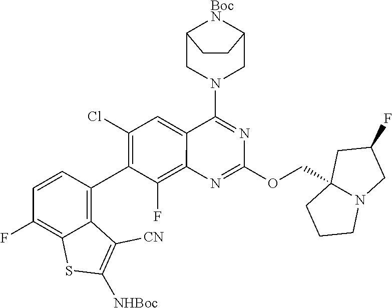 [SMILES: CC(C)(C)OC(=O)Nc1sc2c(F)ccc(-c3c(Cl)cc4c(N5CC6CCC(C5)N6C(=O)OC(C)(C)C)nc(OC[C@@]56CCCN5C[C@H](F)C6)nc4c3F)c2c1C#N]

The mixture of tert-butyl 3-(7-bromo-6-chloro-8-fluoro-2-(((2R,7aS)-2-fluoro tetrahydro-1H-pyrrolizin-7a(5H)-yl)methoxy)quinazolin-4-yl)-3,8-diazabicyclo[3.2.1] octane-8-carboxylate (126 mg, 0.2 mmol), tert-butyl (3-cyano-4-(5,5-dimethyl-1,3,2-dioxaborinan-2-yl)-7-fluorobenzo[b]thiophen-2-yl)carbamate (120 mg, 0.3 mmol) and PdCl₂DPEphos (21 mg, 0.03 mmol), Cs₂CO₃ (196 mg, 0.6 mmol) in toluene (5 mL) was stirred at 100° C. for 15 hrs. After completion, the reaction mixture was purified by Prep-TLC (DCM:MeOH=10:1) to give the titled product (120 mg, crude). MS (ESI, m/e) [M+H]⁺ 840.

### Step 2: 4-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile

To a solution of tert-butyl 3-(7-(2-((tert-butoxycarbonyl)amino)-3-cyano-7-fluoro benzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (120 mg, 0.143 mmol) in DCM (4 mL) was added TFA (2 mL) at room temperature. The resulting mixture was stirred at room temperature for 3 hrs. After completion, the reaction mixture was concentrated and the residue was purified by prep-HPLC to give the title product (16.0 mg, FA salt, 17.6%). ¹H NMR (500 MHz, CD₃OD) δ 8.37 (s, 1H), 7.93 (s, 1H), 7.22-7.19 (m, 1H), 7.07-7.03 (m, 1H), 5.53-5.43 (m, 1H), 4.71-4.46 (m, 4H), 4.05 (s, 2H), 3.86-3.61 (m, 5H), 2.64-2.41 (m, 2H), 2.33-2.23 (m, 3H), 2.13-1.95 (m, 5H). MS (ESI, m/e) [M+H]⁺ 640.5.

### Example 107: 6-(4-(3-oxa-7,9-diazabicyclo[3.3.1]nonan-9-yl)-6-chloro-8-fluoro-2-(((2R,7aS)-2-fluorotetrahydro-1H-pyrrolizin-7a(5H)-yl)methoxy)quinazolin-7-yl)-4-chloro-5-(trifluoromethyl)pyridin-2-amine

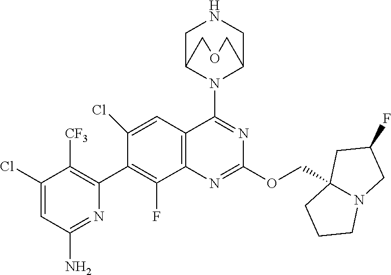 [SMILES: Nc1cc(Cl)c(C(F)(F)F)c(-c2c(Cl)cc3c(N4C5CNCC4COC5)nc(OC[C@@]45CCCN4C[C@H](F)C5)nc3c2F)n1]

Synthetic Route

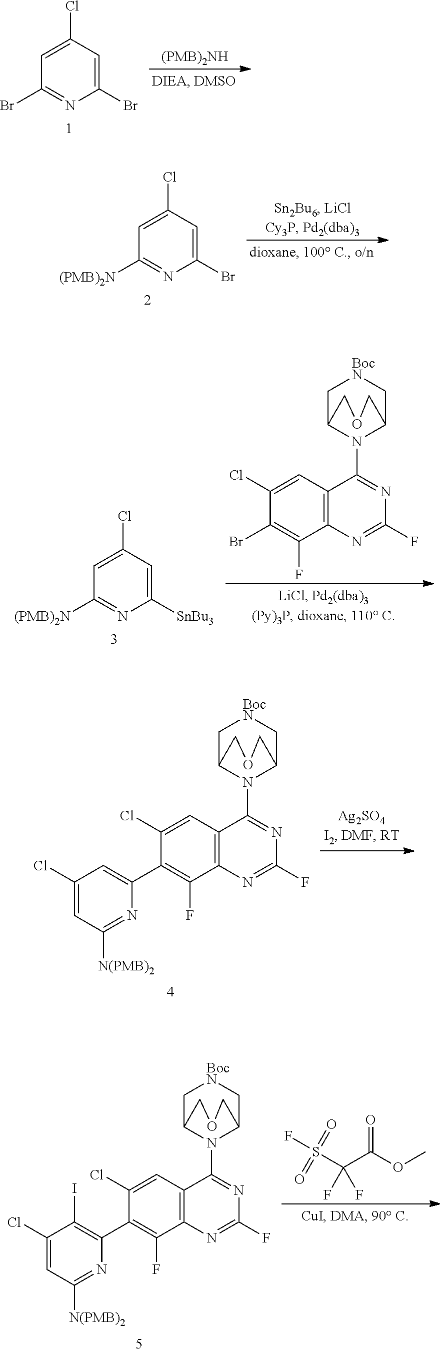

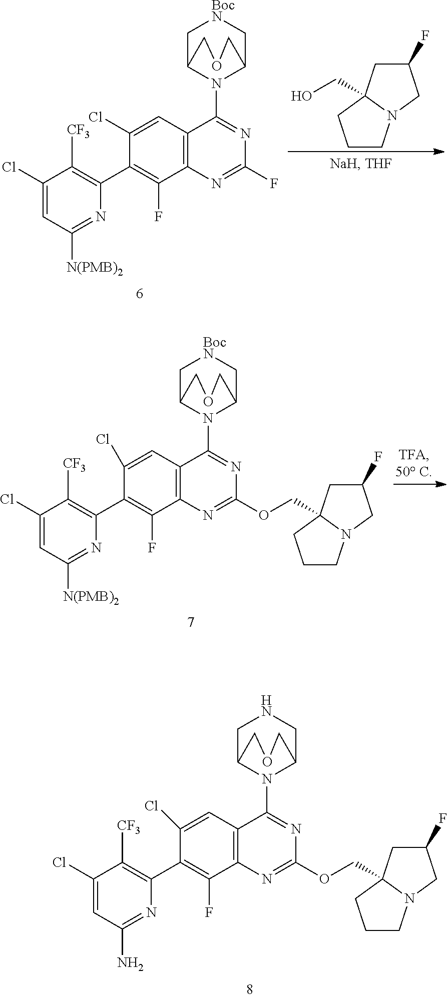

### Step 1: 6-bromo-4-chloro-N,N-bis(4-methoxybenzyl)pyridin-2-amine

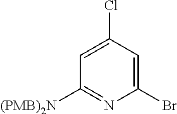

Into a 2000-mL round-bottom flask were placed 2,6-dibromo-4-chloropyridine (34 g, 0.126 mol, 1.00 equiv), DMSO (340 mL), PMB₂NH (30.8 g, 0.119 mol, 0.95 equiv), DIEA (244 g, 1.891 mol, 15.0 equiv). The resulting solution was stirred for one night at 120° C. in an oil bath. The resulting solution was diluted with 2000 mL of water. The resulting solution was extracted with 2000 mL of ethyl acetate. The resulting mixture was washed with 3×800 mL of brine. The resulting mixture was concentrated under vacuum. The crude product was purified by combi-Flash with the following conditions: Column, silica gel; mobile phase, PE:EA=100:0 increasing to PE:EA=75:25 within 1 hr; Detector, 254/280 nm. This resulted in 14 g (25.0% yield) of the title product. MS (ESI, m/e) [M+H]⁺ 447.05.

### Step 2: 4-chloro-N,N-his(4-methoxybenzyl)-6-(tributylstannyl)pyridin-2-amine

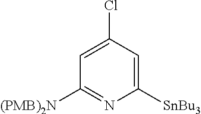

Into a 100-mL round-bottom flask were placed 6-bromo-4-chloro-N,N-bis(4-methoxybenzyl)pyridin-2-amine (2 g, 0.004 mol, 1.00 equiv), 1,4-dioxane (20 mL), Sn₂Bu₆ (3.8 g, 0.006 mol, 1.5 equiv), Cy₃P (0.5 g, 0.002 mol, 0.4 equiv), Pd₂(dba)₃ (0.8 g, 0.001 mol, 0.2 equiv), LiCl (0.37 g, 0.009 mol, 2.0 equiv). The resulting solution was stirred for one night at 90° C. The resulting mixture was concentrated under vacuum. The crude product was purified by combi-Flash with the following conditions: Column, Al₂O₃ gel; mobile phase, PE:EA=100:0 increasing to PE:EA=90:10 within 1 h; Detector, 254/280 nm. This resulted in 1.5 g (51.72% yield) of the title product. MS (ESI, m/e) [M+H]⁺ 658.90.

### Step 3: tert-butyl 9-(7-(6-(bis(4-methoxybenzyl)amino)-4-chloropyridin-2-yl)-6-chloro-2,8-difluoroquinazolin-4-yl)-3-oxa-7,9-diazabicyclo[3.3.1]nonane-7-carboxylate

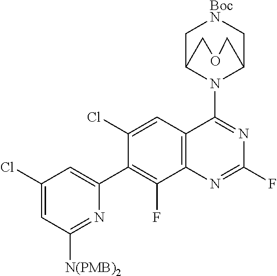

Into a 100-mL round-bottom flask were placed at 4-chloro-N,N-bis(4-methoxybenzyl)-6-(tributylstannyl) pyridin-2-amine (800 mg, 1.185 mmol, 1.20 equiv), 1,4-dioxane (10 mL), tert-butyl 9-(7-bromo-6-chloro-2,8-difluoroquinazolin-4-yl)-3-oxa-7,9-diazabicyclo [3.3.1]nonane-7-carboxylate (500 mg, 0.992 mmol, 1.00 equiv), Cy₃P (111 mg, 0.396 mmol, 0.4 equiv), Pd₂(dba)₃ (181 mg, 0.197 mol, 0.2 equiv), LiCl (83 mg, 1.976 mol, 2.0 equiv). The resulting solution was stirred for one night at 110° C. in an oil bath. The resulting solution was diluted with 50 mL of water. The resulting solution was extracted with EA. The resulting mixture was washed with 3×50 mL of brine. The resulting mixture was concentrated under vacuum. The crude product was purified by combi-Flash with the following conditions: Column, silica gel; mobile phase, PE:EA=100:0 increasing to PE:EA=75:25 within 1 h; Detector, 254/280 nm. This resulted in 350 mg (44.58% yield) of the title product. MS (ESI, m/e) [M+H]⁺ 793.10.

### Step 4: tert-butyl 9-(7-(6-(bis(4-methoxybenzyl)amino)-4-chloro-3-iodopyridin-2-yl)-6-chloro-2,8-difluoroquinazolin-4-yl)-3-oxa-7,9-diazabicyclo[3.3.1]nonane-7-carboxylate

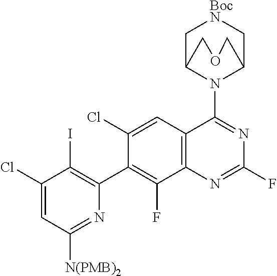

Into a 50-mL round-bottom flask, were placed tert-butyl 9-(7-(6-(bis(4-methoxy benzyl)amino)-4-chloropyridin-2-yl)-6-chloro-2,8-difluoroquinazolin-4-yl)-3-oxa-7,9-diaza bicyclo[3.3.1]nonane-7-carboxylate (320 mg, 0.404 mmol, 1.00 equiv), DMF (3 mL). This was followed by the addition of Ag₂SO₄ (164 mg, 0.807 mmol, 2.0 equiv) in portions at rt in 10 min. The resulting solution was stirred for 5 min at room temperature. To this was added I2 (123 mg, 0.484 mmol, 1.2 equiv) at rt. The resulting solution was allowed to react with stirring for 2 h at room temperature. The resulting solution was diluted with 100 mL of EA. The resulting mixture was filtered, the filter cake was washed with EA. The resulting solution was washed with 3×50 mL of brine. The resulting mixture was concentrated under vacuum. The crude product was purified by combi-Flash with the following conditions: Column, silica gel; mobile phase, PE:EA=100:0 increasing to PE:EA=75:25 within 1 h; Detector, 254/280 nm. This resulted in 140 mg (37.8% yield) of the title product. MS (ESI, m/e) [M+H]⁺ 920.85.

### Step 5: tert-butyl 9-(7-(6-(bis(4-methoxybenzyl)amino)-4-chloro-3-(trifluoro methyl)pyridin-2-yl)-6-chloro-2,8-difluoroquinazolin-4-yl)-3-oxa-7,9-diazabicyclo[3.3.1] nonane-7-carboxylate

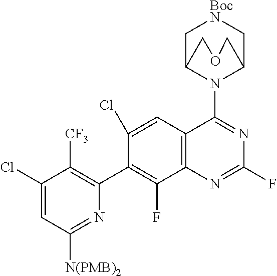

Into a 50-mL round-bottom flask was placed tert-butyl 9-(7-(6-(bis(4-methoxybenzyl)amino)-4-chloro-3-iodopyridin-2-yl)-6-chloro-2,8-difluoroquinazolin-4-yl)-3-oxa-7,9-diazabicyclo[3.3.1]nonane-7-carboxylate (140 mg, 0.152 mmol, 1.00 equiv), DMA (3 mL), CuI (112 mg, 0.589 mmol, 4 equiv), methyl 2,2-difluoro-2-sulfoacetate (294 mg, 1.531 mmol, 20 equiv). The resulting solution was stirred 2 h at 90° C. in an oil bath. The resulting solution was diluted with 50 mL of EA. The resulting mixture was filtered, the filter cake was washed with EA. The resulting solution was washed with 3×50 mL of brine. The resulting mixture was concentrated under vacuum. The crude product was purified by combi-Flash with the following conditions: Column, silica gel; mobile phase, PE:EA=100:0 increasing to PE:EA=65:35 within 1 h; Detector, 254/280 nm. This resulted in 64 mg (48.85% yield) of the title product. MS (ESI, m/e) [M+H]⁺ 861.25.

### Step 6: tert-butyl 9-(7-(6-(bis(4-methoxybenzyl)amino)-4-chloro-3-(trifluoro methyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3-oxa-7,9-diazabicyclo[3.3.1]nonane-7-carboxylate

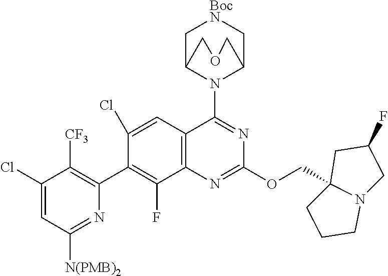

To a stirred solution of tert-butyl 9-(7-(6-(bis(4-methoxybenzyl)amino)-4-chloro-3-(trifluoromethyl)pyridin-2-yl)-6-chloro-2,8-difluoroquinazolin-4-yl)-3-oxa-7,9-diazabicyclo [3.3.1]nonane-7-carboxylate (54 mg, 0.062 mmol, 1.00 equiv) and ((2R,7aS)-2-fluorotetrahydro-1H-pyrrolizin-7a(5H)-yl)methanol (12 mg, 0.075 mmol, 1.2 equiv) in THF (2 mL) were added NaH (60% in oil, 5 mg, 0.125 mmol, 2.0 equiv) in portions at 0° C. The resulting solution was stirred for 2 h at rt. The reaction was quenched with water at 0° C. The resulting solution was diluted with 100 mL of water. The resulting solution was extracted with EA. The resulting mixture was washed with 3×20 mL of brine. The resulting mixture was concentrated under vacuum. The residue was purified by Prep-TLC (PE/EA=1:1) to afford the title product (40 mg, 54.1% yield). MS (ESI, m/e) [M+H]⁺ 1000.20.

### Step 7: 6-(4-(3-oxa-7,9-diazabicyclo[3.3.1]nonan-9-yl)-6-chloro-8-fluoro-2-(((2R,7aS)-2-fluorotetrahydro-1H-pyrrolizin-7a(5H)-yl)methoxy)quinazolin-7-yl)-4-chloro-5-(trifluoromethyl)pyridin-2-amine

The resulting mixture of tert-butyl 9-(7-(6-(bis(4-methoxybenzyl)amino)-4-chloro-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3-oxa-7,9-diazabicyclo[3.3.1]nonane-7-carboxylate (40 mg, 0.040 mmol, 1.0 equiv) in TFA (1 mL) was stirred for 5 h at 50° C. The resulting mixture was concentrated under reduced pressure. The crude product (10 mg) was purified by Prep-HPLC to give the title product (9.3 mg, TFA salt, 35.7% yield). ¹H NMR (300 MHz, CD₃OD) δ 7.81 (d, J=1.7 Hz, 1H), 6.85 (d, J=0.9 Hz, 1H), 5.70-5.41 (m, 1H), 4.76 (s, 2H), 4.69 (s, 2H), 4.32-4.10 (m, 4H), 4.14-4.00 (m, 1H), 3.99-3.60 (m, 6H), 3.47 (td, J=10.6, 5.7 Hz, 1H), 2.90-2.10 (m, 6H). MS (ESI, m/e) [M+H]⁺ 660.15.

### Example 108: 6-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-4-ethynyl-5-(trifluoromethyl)pyridin-2-aminp

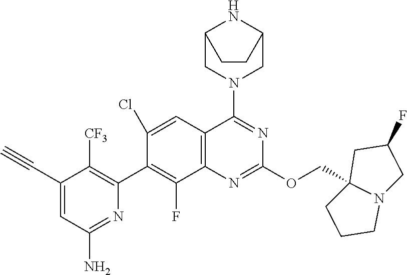 [SMILES: C#Cc1cc(N)nc(-c2c(Cl)cc3c(N4CC5CCC(C4)N5)nc(OC[C@@]45CCCN4C[C@H](F)C5)nc3c2F)c1C(F)(F)F]

Synthetic Route

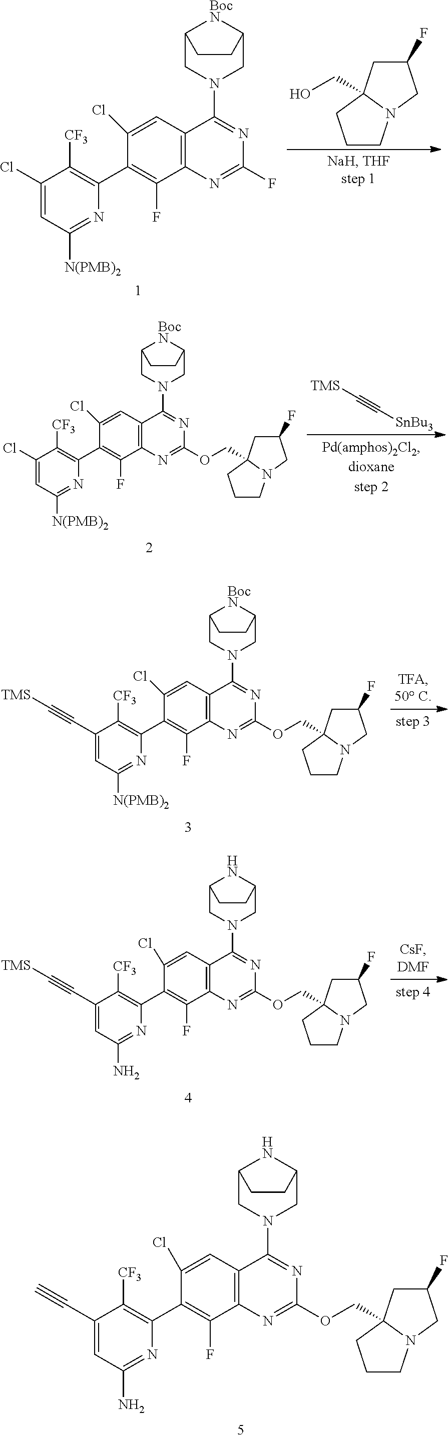

### Step 1: tert-butyl 3-(7-(6-(bis(4-methoxybenzyl)amino)-4-chloro-3-(trifluoro methyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

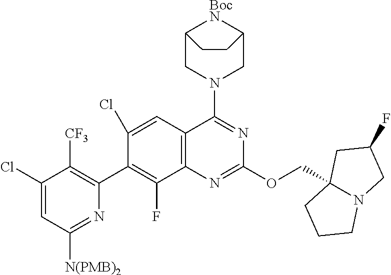

To a solution of ((2R,7aS)-2-fluoro-hexahydropyrrolizin-7a-yl)methanol (124 mg, 0.78 mmol) in THF (8 mL) was added sodium hydride (60% in oil, 42 mg, 1.08 mmol) at 0° C. The mixture was stirred for 15 min. tert-butyl 3-(7-(6-(bis((4-methoxyphenyl)methyl)amino)-4-chloro-3-(trifluoromethyl)pyridin-2-yl)-6-chloro-2,8-difluoroquinazolin-4-yl)-3,8-diazabicyclo [3.2.1]octane-8-carboxylate (600 mg, 0.70 mmol) was added and the mixture was allowed to warm to rt and stirred for 2 h. The reaction mixture was quenched by water and extracted with EtOAc. The resulting mixture was washed with brine and concentrated under reduced pressure. The residue was purified by Prep-TLC (PE /EA=1:1) to give the title product (450 mg, 64% yield). MS (ESI, m/e) [M+H]⁺ 845.35.

### Step 2: tert-butyl 3-(7-(6-(bis(4-methoxybenzyl)amino)-3-(trifluoromethyl)-4-((trimethylsilyl)ethynyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

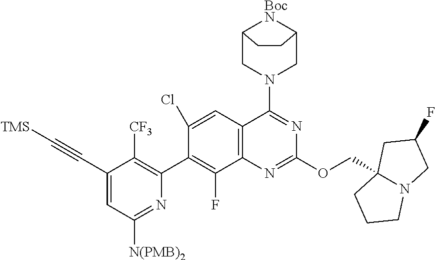

To a stirred solution of tert-butyl 3-(2-(((2R,7aS)-2-fluoro-hexahydropyrrolizin-7a-yl)methoxy)-7-(6-(bis((4-methoxyphenyl)methyl]amino)-4-chloro-3-(trifluoromethyl) pyridin-2-yl)-6-chloro-8-fluoroquinazolin-4-yl)-3,8-diazabicyclo[3.2.1]octane-8-carboxylate (390 mg, 0.39 mmol) and trimethyl(2-(tributylstannyl)ethynyl)silane (460 mg, 1.18 mmol) in 1,4-dioxane (8 mL) was added Pd(amphos)₂Cl₂ (56.08 mg, 0.07 mmol) in portions. The resulting mixture was stirred for 4 h at 100° C. The resulting mixture was concentrated under reduced pressure. The residue was purified by Prep-TLC (PE/EA=1:1) to give the title product (350 mg, 84% yield). MS (ESI, m/e) [M+H]⁺ 1046.45.

### Step 3: 6-(4-((1R,5S)-3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-5-(trifluoro methyl)-4-((trimethylsilyl)ethynyl)pyridin-2-amine

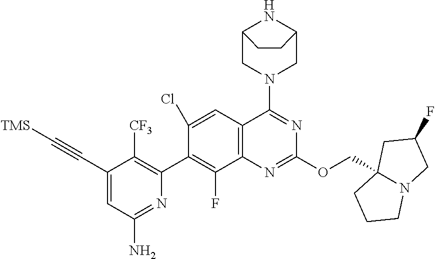 [SMILES: C[Si](C)(C)C#Cc1cc(N)nc(-c2c(Cl)cc3c(N4CC5CCC(C4)N5)nc(OC[C@@]45CCCN4C[C@H](F)C5)nc3c2F)c1C(F)(F)F]

A solution of tert-butyl 3-(2-(((2R,7aS)-2-fluoro-hexahydropyrrolizin-7a-yl)methoxy)-7-(6-(bis((4-methoxyphenyl)methyl)amino)-3-(trifluoromethyl)-4-(2-(trimethyl silyl)ethynyl)pyridin-2-yl)-6-chloro-8-fluoroquinazolin-4-yl)-3,8-diazabicyclo [3.2.1]octane-8-carboxylate (330 mg, 0.31 mmol) in TFA (5 mL) was stirred for 2 h at 50° C. The mixture was basified to pH>7 with saturated Na₂CO₃ (aq.). The resulting mixture was extracted with EtOAc. The combined organic layers were washed with brine, dried over anhydrous Na₂SO₄. After filtration, the filtrate was concentrated under reduced pressure. The crude product was directly used in the next step without further purification. MS (ESI, m/e) [M+H]⁺ 706.30.

### Step 4: 6-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-4-ethynyl-5-(trifluoro methyl)pyridin-2-amine

To a stirred mixture of 6-(2-(((2R,7aS)-2-fluoro-hexahydropyrrolizin-7a-yl)methoxy)-6-chloro-4-(3,8-diazabicyclo[3.2.1]octan-3-yl)-8-fluoroquinazolin-7-yl)-5-(trifluoromethyl)-4-(2-(trimethylsilyl) ethynyl)pyridin-2-amine (210 mg, 0.29 mmol) in DMF (5 mL) was added CsF (90 mg, 0.59 mmol) in portions at rt. The resulting mixture was stirred for 2 h at rt. The resulting mixture was extracted with EtOAc. The combined organic layers were washed with brine, dried over anhydrous Na₂SO₄. After filtration, the filtrate was concentrated under reduced pressure. The residue was purified to give the titled product (19.1 mg, 10% yield). ¹H NMR (400 MHz, CD₃OD) δ 7.85 (d, J=1.7 Hz, 1H), 6.87 (d, J=0.9 Hz, 1H), 5.32 (d, J=53.7 Hz, 1H), 4.54-4.40 (m, 2H), 4.29 (d, J=10.7 Hz, 1H), 4.22 (d, J=10.5 Hz, 1H), 4.10 (s, 1H), 3.64 (m, 4H), 3.59 (m, 3H), 3.09-2.98 (m, 1H), 2.38 (m, J=15.1, 4.7 Hz, 2H), 2.27 (td, J=10.2, 5.1 Hz, 1H), 2.24-2.11 (m, 2H), 1.85 (m, 5H). MS (ESI, m/e) [M+H]⁺ 634.05.

### Example 109: 2-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-6-amino-3-(trifluoromethyl)isonicotinonitrile

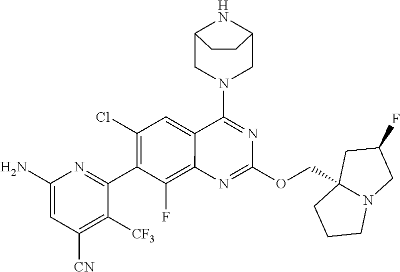 [SMILES: N#Cc1cc(N)nc(-c2c(Cl)cc3c(N4CC5CCC(C4)N5)nc(OC[C@@]45CCCN4C[C@H](F)C5)nc3c2F)c1C(F)(F)F]

Synthetic Route

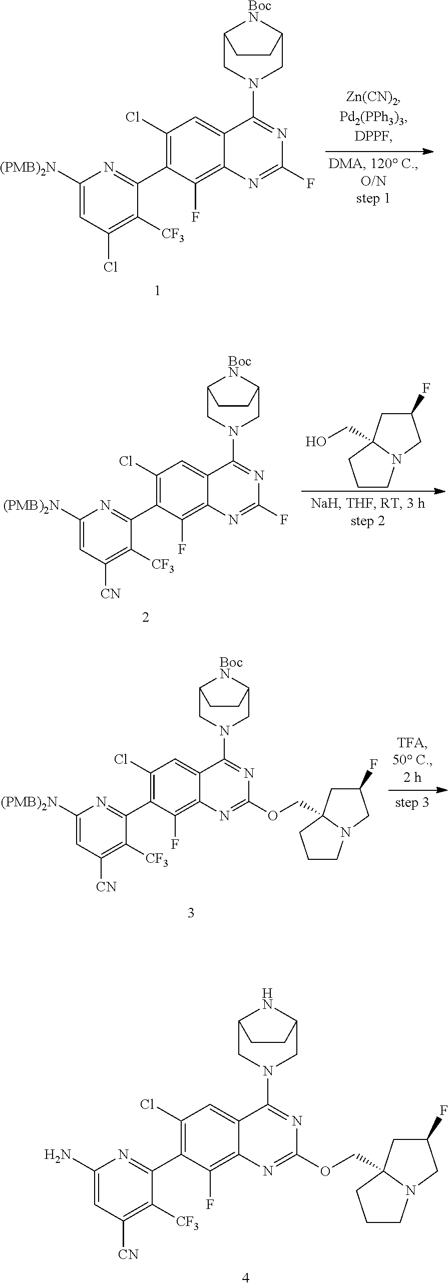

### Step 1: tert-butyl 3-(7-(6-(bis(4-methoxybenzyl)amino)-4-cyano-3-(trifluoro methyl)pyridin-2-yl)-6-chloro-2,8-difluoroquinazolin-4-yl)-3,8-diazabicyclo[3.2.1]octane-8-carboxylate

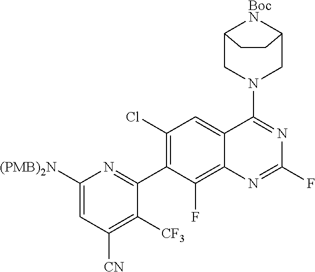

Into a 50-mL round-bottom flask purged and maintained with an inert atmosphere of nitrogen, was placed tert-butyl 3-(7-(6-(bis(4-methoxybenzyl)amino)-4-chloro-3-(trifluoro methyl)pyridin-2-yl)-6-chloro-2,8-difluoroquinazolin-4-yl)-3,8-diazabicyclo[3.2.1] octane-8-carboxylate (240 mg, 0.284 mmol, 1.00 equiv), DMA (4 mL), Zn(CN)₂ (49.4 mg, 0.426 mmol, 1.5 equiv), Pd₂(dba)₃ (52.0 mg, 0.057 mmol, 0.2 equiv), DPPF (62.9 mg, 0.114 mmol, 0.4 equiv). The resulting solution was stirred for 18 h at 120° C. in an oil bath. The resulting solution was diluted with 50 mL of water. The resulting solution was extracted with 100 mL of EA. The resulting mixture was washed with 3×40 mL brine. The resulting mixture was concentrated under vacuum. The crude product was purified by Prep-TLC (PE:EA=3:1). This resulted in 140 mg (58.9% yield) of the title product. MS (ESI, m/e) [M+H]⁺ 836.

### Step 2: tert-butyl 3-(7-(6-(bis(4-methoxybenzyl)amino)-4-cyano-3-(trifluoro methyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

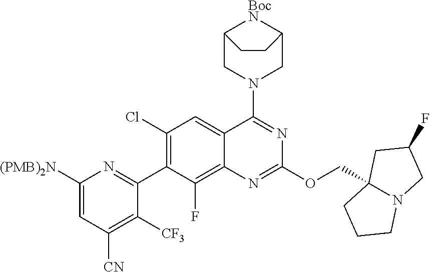

To a stirred solution of tert-butyl 3-(7-(6-(bis(4-methoxybenzyl)amino)-4-cyano-3-(trifluoromethyl)pyridin-2-yl)-6-chloro-2,8-difluoroquinazolin-4-yl)-3,8-diazabicyclo[3.2.1] octane-8-carboxylate (140 mg, 0.167 mmol, 1.00 equiv) and ((2R,7aS)-2-fluorotetrahydro-1H-pyrrolizin-7a(5H)-yl)methanol (53.3 mg, 0.335 mmol, 2 equiv) in THF (4 mL) were added NaH (60% in oil, 20 mg, 0.501 mmol, 3 equiv) in portions at 0° C. The resulting solution was stirred for 2 h at rt. The reaction was quenched with water at 0° C., diluted with 50 mL of water, extracted with EA, washed with 3×50 mL brine and concentrated under vacuum. The crude product was purified by Prep-TLC (PE:EA=1:1) to give 60 mg (36.8% yield) of the title product. MS (ESI, m/e) [M+H]⁺ 975.

### Step 3: 2-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-6-amino-3-(trifluoromethyl) isonicotinonitrile

A solution of tert-butyl 3-(7-(6-(bis(4-methoxybenzyl)amino)-4-cyano-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (60 mg, 0.061 mmol, 1.0 equiv) in TFA (1 mL) was stirred for 2 h at 50° C. The resulting mixture was concentrated under vacuum. The residue was dissolved in EA. The organic layer was basified by Na₂CO₃ (aq.). The resulting mixture was stirred for 30 min at rt, diluted with 50 mL of water and 50 mL of EA. The aqueous phase was extracted with EA. The combined organic layers were dried over anhydrous Na₂SO₄. After filtration, the filtrate was concentrated under reduced pressure. The residue (50 mg crude) was purified by Prep-HPLC to give the titled product (16.4 mg, 42.1% yield). ¹H NMR (400 MHz, CD₃OD) δ 7.87 (d, J=1.7 Hz, 1H), 7.09 (s, 1H), 5.43-5.21 (m, 1H), 4.62 (s, 1H), 4.52-4.40 (m, 2H), 4.30-4.18 (m, 2H), 3.63 (q, J=5.9, 5.3 Hz, 4H), 3.27-3.16 (m, 2H), 3.02 (m, 1H), 2.41-2.19 (m, 2H), 2.18-2.10 (m, 1H), 2.07-1.92 (m, 2H), 1.83 (s, 5H). MS (ESI, m/e) [M+H]⁺ 635.05.

### Example 110: 3-(4-(3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-fluoro-4-(trifluoromethyl)phenol

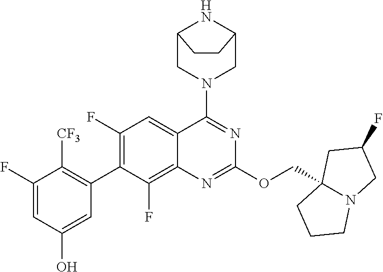 [SMILES: Oc1cc(F)c(C(F)(F)F)c(-c2c(F)cc3c(N4CC5CCC(C4)N5)nc(OC[C@@]45CCCN4C[C@H](F)C5)nc3c2F)c1]

Synthetic Route

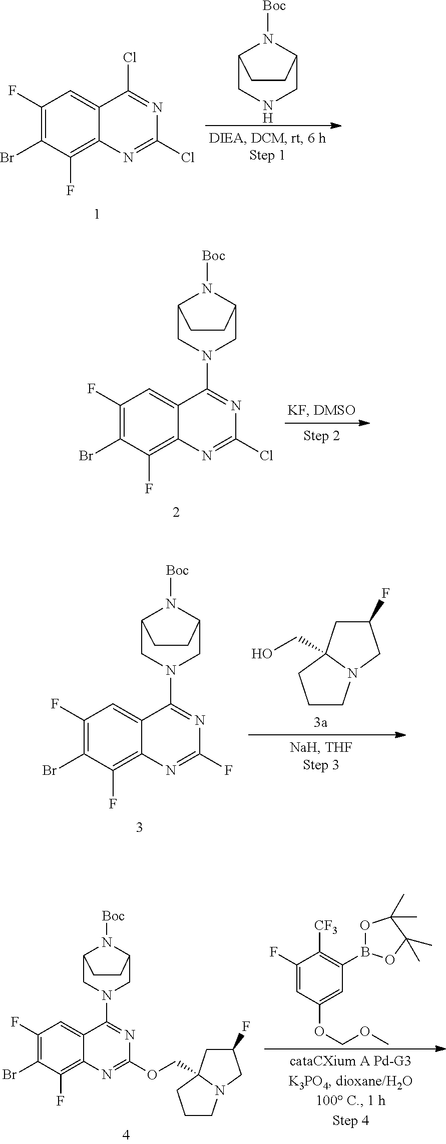

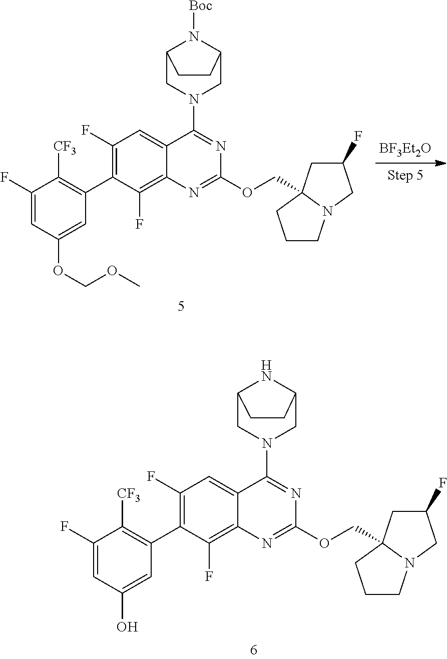 [SMILES: COCOc1cc(F)c(C(F)(F)F)c(-c2c(F)cc3c(N4CC5CCC(C4)N5C(=O)OC(C)(C)C)nc(OC[C@@]45CCCN4C[C@H](F)C5)nc3c2F)c1 ; Oc1cc(F)c(C(F)(F)F)c(-c2c(F)cc3c(N4CC5CCC(C4)N5)nc(OC[C@@]45CCCN4C[C@H](F)C5)nc3c2F)c1]

### Step 1: tert-butyl 3-(7-bromo-2-chloro-6,8-difluoroquinazolin-4-yl)-3,8-diazabicyclo [3.2.1]octane-8-carboxylate

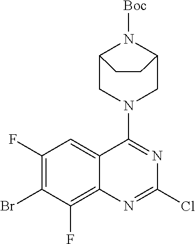 [SMILES: CC(C)(C)OC(=O)N1C2CCC1CN(c1nc(Cl)nc3c(F)c(Br)c(F)cc13)C2]

To a solution of 7-bromo-2,4-dichloro-6,8-difluoroquinazoline (1 g, 3.2 mmol) in DCM (25 mL) was added tert-butyl 3,8-diazabicyclo[3.2.1]octane-8-carboxylate (700 mg, 3.3 mmol) and DIPEA (1.28 g, 9.9 mmol), stirred at room temperature for 6 hrs. The reaction solution was concentrated and purified by combi-flash (PE/EA=3/1) to give the titled product (1.56 g, 99% yield). MS (ESI, m/e) [M+H]⁺ 488.8, 490.8.

### Step 2: tert-butyl 3-(7-bromo-2,6,8-trifluoroquinazolin-4-yl)-3,8-diazabicyclo [3.2.1]octane-8-carboxylate

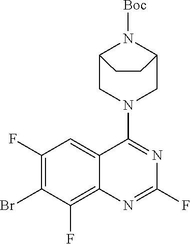 [SMILES: CC(C)(C)OC(=O)N1C2CCC1CN(c1nc(F)nc3c(F)c(Br)c(F)cc13)C2]

To a solution of tert-butyl 3-(7-bromo-2-chloro-6,8-difluoroquinazolin-4-yl)-3,8-diazabicyclo[3.2.1]octane-8-carboxylate (800 mg, 1.65 mmol) in DMSO (25 mL) was added KF (1.43 g, 24.7 mmol) and stirred at 100° C. for 16 hrs. The cooled resulting mixture was diluted with water (50 mL) and extracted with DCM (30 mL) for 3 times. The combined DCM phase was washed with brine (50 mL) for 2 times and dried over Na₂SO₄, concentrated to give a residue (1.5 g) which was directly used in next step. MS (ESI, m/e) [M+1]⁺473.3, 475.3.

### Step 3: tert-butyl 3-(7-bromo-6,8-difluoro-2-(((2R,7aS)-2-fluorotetrahydro-1H-pyrrolizin-7a(5H)-yl)methoxy)quinazolin-4-yl)-38-diazabicyclo[3.2.1]octane-8-carboxylate

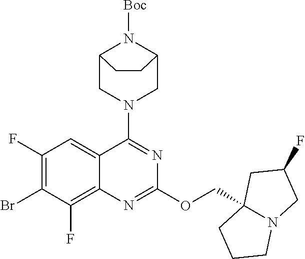 [SMILES: CC(C)(C)OC(=O)N1C2CCC1CN(c1nc(OC[C@@]34CCCN3C[C@H](F)C4)nc3c(F)c(Br)c(F)cc13)C2]

To a solution of ((2R,7aS)-2-fluorotetrahydro-1H-pyrrolizin-7a(5H)-yl)methanol (405 mg, 2.55 mmol) in THF (20 mL) was added 60% NaH (214 mg, 5.35 mmol) and stirred at 0° C. for 0.5 hr. The resulting solution was added tert-butyl 3-(7-bromo-2,6,8-trifluoro quinazolin-4-yl)-3,8-diazabicyclo[3.2.1]octane-8-carboxylate (1.24 g, crude) and stirred at 0° C. for another 1 hr. The resulting solution was added water (40 mL) and extracted with EA (20 mL) for 2 times. The combined EA phase was dried over Na₂SO₄ and concentrated to give a residue which was further purified by combi-flash (DCM/MeOH=10/1) to give the title product (448 mg, 54% yield). MS (ESI, m/e) [M+1]⁺612.4, 614.5.

### Step 4: tert-butyl 3-(6,8-difluoro-7-(3-fluoro-5-(methoxymethoxy)-2-(trifluoro methyl)phenyl)-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

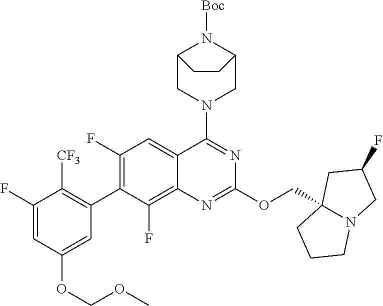 [SMILES: COCOc1cc(F)c(C(F)(F)F)c(-c2c(F)cc3c(N4CC5CCC(C4)N5C(=O)OC(C)(C)C)nc(OC[C@@]45CCCN4C[C@H](F)C5)nc3c2F)c1]

To a mixture of 2-(3-fluoro-5-(methoxymethoxy)-2-(trifluoromethyl)phenyl)-4,4,5,5-tetramethyl-1,3,2-dioxaborolane (200 mg, 0.57 mmol), tert-butyl 3-(7-bromo-6,8-difluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (100 mg, 0.16 mmol), cataCXium-A-Pd-G3 (40 mg, 0.055 mmol) and K₃PO₄ (100 mg, 0.47 mmol) was added 1,4-dioxane (20 mL) and H₂O (4 mL), and the resulting mixture was stirred at 100° C. for 2 hrs. After completion, the reaction mixture was diluted with water (20 mL) and extracted with DCM (15 mL) for 3 times. The combined DCM phase was dried over Na₂SO₄, concentrated to give a residue which was purified by Prep-TLC (DCM/MeOH=10/1) to give the title product (95 mg, 79% yield). MS (ESI, m/e) [M+1]⁺756.6.

### Step 5: 3-(4-(3,8-diazabicyclo[3.2.1]octan-3-yl)-6,8-difluoro-2-(((2R,7aS)-2-fluorotetrahydro-1H-pyrrolizin-7a(5H)-yl)methoxy)quinazolin-7-yl)-5-fluoro-4-(trifluoromethyl) phenol

A solution of tert-butyl 3-(6,8-difluoro-7-(3-fluoro-5-(methoxymethoxy)-2-(trifluoromethyl)phenyl)-2-(((2R,7aS)-2-fluorotetrahydro-1H-pyrrolizin-7a(5H)-yl)methoxy) quinazolin-4-yl)-3,8-diazabicyclo[3.2.1]octane-8-carboxylate (95 mg, 0.13 mmol) in DCM (10 mL) was added BF₃-Et₂O (100 mg, 0.7 mmol) and stirred at room temperature for 2 hrs, the resulting solution was added MeOH (5 mL), concentrated and purified by Prep-HPLC to give the title product (25 mg, FA salt, 29% yield). ¹H NMR (500 MHz, CD₃OD) δ 8.49 (s, 1H), 7.61 (d, J=9.7 Hz, 1H), 6.83 (d, J=12.9 Hz, 1H), 6.60 (s, 1H), 5.41 (d, J=52.9 Hz, 1H), 4.56-4.36 (m, 4H), 3.91 (s, 2H), 3.71-3.44 (m, 5H), 3.23-3.16 (m, 1H), 2.52-2.33 (m, 2H), 2.28-2.24 (m, 1H), 2.17-2.11 (m, 2H), 2.05-1.93 (m, 5H). MS (ESI, m/e) [M+H]⁺ 612.5.

### Example 111: 4-(4-(3,8-diazabicyclo[3.2.1]octan-3-yl)-8-fluoro-2-(((2S,4R)-4-fluoro-1-methylpyrrolidin-2-yl)methoxy)-6-(trifluoromethyl)quinazolin-7-yl)-2-amino-7-fluoro benzo[b]thiophene-3-carbonitrile

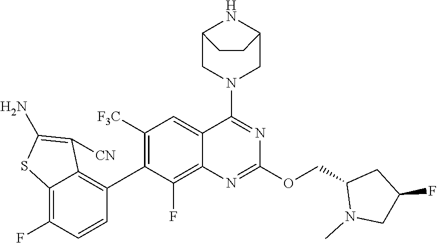 [SMILES: CN1C[C@H](F)C[C@H]1COc1nc(N2CC3CCC(C2)N3)c2cc(C(F)(F)F)c(-c3ccc(F)c4sc(N)c(C#N)c34)c(F)c2n1]

Synthetic Route

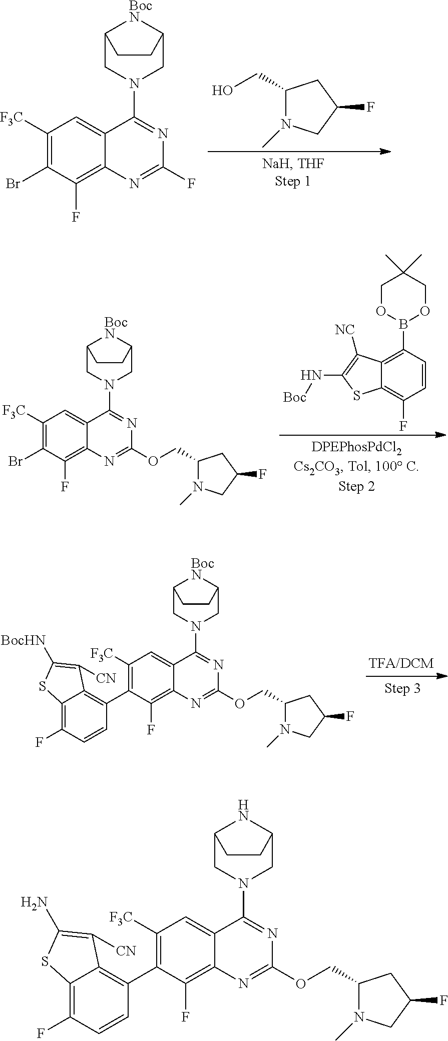

### Step 1: tert-butyl 3-(7-bromo-8-fluoro-2-(((2S,4R)-4-fluoro-1-methylpyrrolidin-2-yl)methoxy)-6-(trifluoromethyl)quinazolin-4-yl)-3,8-diazabicyclo [3.2.1]octane-8-carboxylate

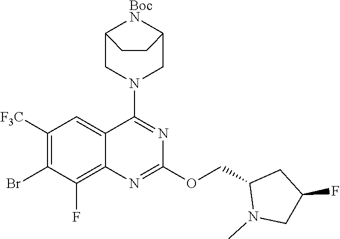 [SMILES: CN1C[C@H](F)C[C@H]1COc1nc(N2CC3CCC(C2)N3C(=O)OC(C)(C)C)c2cc(C(F)(F)F)c(Br)c(F)c2n1]

To a solution of ((2S,4R)-4-fluoro-1-methylpyrrolidin-2-yl)methanol (319.4 mg, 2.4 mmol) in THF (10 mL) was added sodium hydride (96.0 mg, 2.4 mmol) at room temperature. The resulting mixture was stirred at room temperature for 1 hrs. Then tert-butyl 3-(7-bromo-2,8-difluoro-6-(trifluoromethyl)quinazolin-4-yl)-3,8-diazabicyclo[3.2.1]octane-8-carboxylate (1044.2 mg, 2.0 mmol) was added to the reaction mixture and stirred at room temperature for 1 hrs. After completion. The reaction mixture was quenched with water (50 mL), diluted with EA (50 mL), washed with brine (50 mL×3). The organic layer was dried over anhydrous Na₂SO₄, filtered and the filtrate was concentrated to give the residue. The residue was purified by silica gel column chromatography, eluted with 0-100% ethyl acetate in petroleum ether to give the title product (1000.0 mg, 1.57 mmol). MS (ESI, m/e) [M+1]⁺636.2, 638.2.

### Step 2: tert-butyl 3-(7-(2-((tert-butoxycarbonyl)amino)-3-cyano-7-fluorobenzo [b]thiophen-4-yl)-8-fluoro-2-(((2S,4R)-4-fluoro-1-methylpyrrolidin-2-yl)methoxy)-6-(trifluoro methyl)quinazolin-4-yl)-3,8-diazabicyclo[3.2.1]octane-8-carboxylate

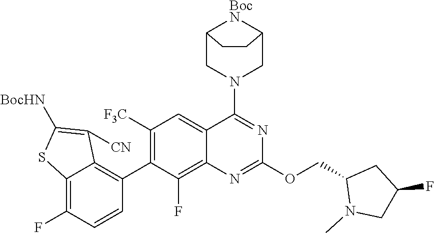 [SMILES: CN1C[C@H](F)C[C@H]1COc1nc(N2CC3CCC(C2)N3C(=O)OC(C)(C)C)c2cc(C(F)(F)F)c(-c3ccc(F)c4sc(NC(=O)OC(C)(C)C)c(C#N)c34)c(F)c2n1]

To a solution of tert-butyl 3-(7-bromo-8-fluoro-2-(((2S,4R)-4-fluoro-1-methylpyrrolidin-2-yl)methoxy)-6-(trifluoromethyl)quinazolin-4-yl)-3,8-diazabicyclo[3.2.1] octane-8-carboxylate (317.6 mg, 0.5 mmol), tert-butyl (3-cyano-4-(5,5-dimethyl-1,3,2-dioxaborinan-2-yl)-7-fluorobenzo[b]thiophen-2-yl)carbamate (404.1 mg, 1.0 mmol) and DPEPhosPdCl₂ (71.6 mg, 0.1 mmol) in Tol (15 mL) was added Cs₂CO₃ (325.8 mg, 1.0 mmol) at room temperature, The resulting mixture was stirred at 100° C. for 16 hrs. After completion. The reaction mixture was diluted with EA (30 mL), washed with brine (250 mL×3). The organic layer was dried over anhydrous Na₂SO₄, filtered and the filtrate was concentrated to give the residue. The residue was purified by Prep-TLC (pure EA) to give the title product (42.4 mg, 0.05 mmol). MS (ESI, m/e) [M+1]⁺848.3.

### Step 3: 4-(4-(3,8-diazabicyclo[3.2.1]octan-3-yl)-8-fluoro-2-(((2S,4R)-4-fluoro-1-methylpyrrolidin-2-yl)methoxy)-6-(trifluoromethyl)quinazolin-7-yl)-2-amino-7-fluorobenzo [b]thiophene-3-carbonitrile

To a solution of tert-butyl 3-(7-(2-((tert-butoxycarbonyl)amino)-3-cyano-7-fluoro benzo[b]thiophen-4-yl)-8-fluoro-2-(((2S,4R)-4-fluoro-1-methylpyrrolidin-2-yl)methoxy)-6-(trifluoromethyl)quinazolin-4-yl)-3,8-diazabicyclo[3.2.1]octane-8-carboxylate (42.4 mg, 0.05 mmol) in DCM (5 mL) was added TFA (5 mL) at room temperature. The resulting mixture was stirred at room temperature for 3 hrs. After completion. The reaction mixture was concentrated to give the residue. The residue was purified by High-HPLC to give the title product (8.86 mg). ¹H NMR (500 MHz, DMSO-d₆) δ 8.14-8.10 (m, 3H), 7.24-7.22 (m, 1H), 7.17-7.14 (m, 1H), 5.53-5.42 (m, 1H), 4.81-4.60 (m, 4H), 4.24-4.19 (m, 3H), 3.98-3.95 (m, 2H), 3.79-3.77 (m, 1H), 3.62-3.52 (m, 2H), 3.07-3.03 (m, 3H), 2.52-2.50 (m, 2H), 1.95-1.84 (m, 4H). MS (ESI, m/e) [M+1]⁺648.4.

### Example 112: 4-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-6-methyl-5-(trifluoromethyl)pyrimidin-2-amine

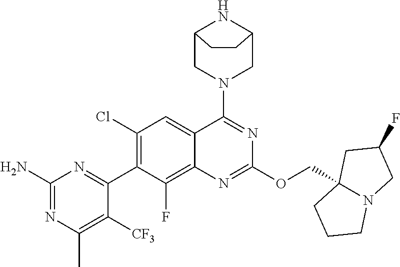 [SMILES: Cc1nc(N)nc(-c2c(Cl)cc3c(N4CC5CCC(C4)N5)nc(OC[C@@]45CCCN4C[C@H](F)C5)nc3c2F)c1C(F)(F)F]

Synthetic Route

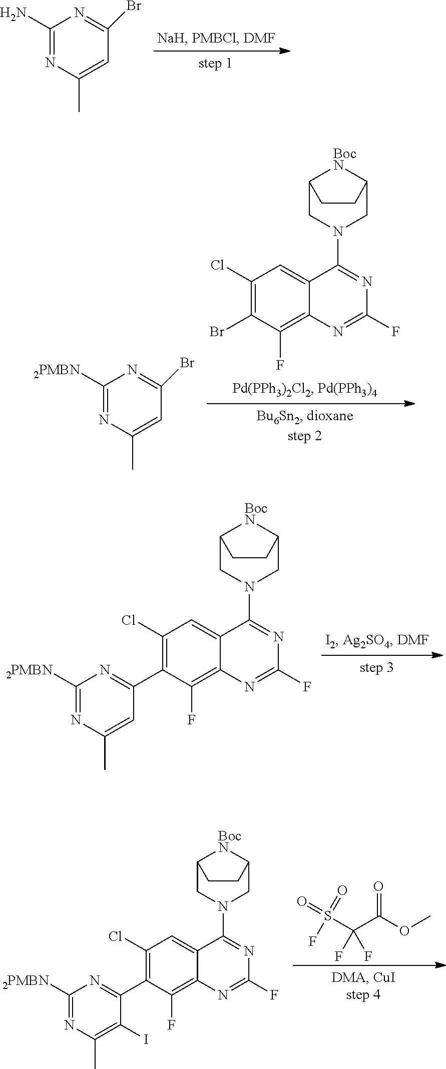

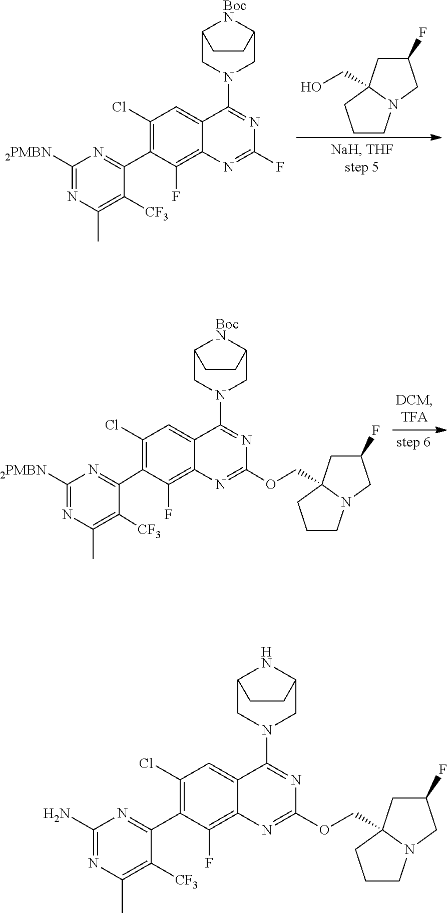

### Step 1: 4-bromo-N,N-bis(4-methoxybenzyl)-6-methylpyrimidin-2-amine

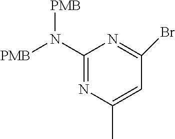

To a solution of 4-bromo-6-methylpyrimidin-2-amine (5.0 g, 26.7 mmol) in DMF (50 mL) was added NaH (3.2 g, 80.2 mmol). The mixture was stirred for 1 h at room temperature and then PMBCl (9.18 g, 58.8 mmol) was added. The reaction mixture was stirred for 2 hours at room temperature. After completion, the reaction was quenched by H₂O. The mixture was poured into water and filtered to obtain solid as the title product (10 g, 23.4 mmol). MS (ESI, m/e) [M+H]⁺ 428.1, 430.1.

### Step 2: tert-butyl 3-(7-(2-(bis(4-methoxybenzyl)amino)-6-methylpyrimidin-4-yl)-6-chloro-2,8-difluoroquinazolin-4-yl)-3,8-diazabicyclo[3.2.1]octane-8-carboxylate

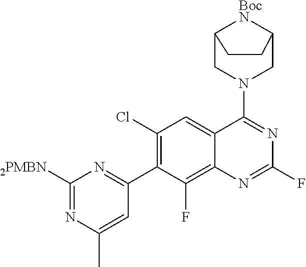

A mixture of 4-bromo-N,N-bis(4-methoxybenzyl)-6-methylpyrimidin-2-amine (1.28 g, 3 mmol), tert-butyl 3-(7-bromo-6-chloro-2,8-difluoroquinazolin-4-yl)-3,8-diazabicyclo [3.2.1]octane-8-carboxylate (976 mg, 2.0 mmol), Pd(PPh₃)₄ (230.8 mg, 0.2 mmol), Pd(PPh₃)₂Cl₂ (140.0 mg, 0.2 mmol), Sn₂(ⁿBu)₆ (1.75 g, 3 mmol) in dioxane (20 mL) was stirred for 16 hours at 110° C. After completion, the resulting mixture was directly concentrated. Then the crude product was further purified by silica column (eluting with PE/EtOAc=3/2) to give the title product (400 mg, 0.53 mmol). MS (ESI, m/e) [M+1]⁺758.3.

### Step 3: tert-butyl 3-(7-(2-(bis(4-methoxybenzyl)amino)-5-iodo-6-methyl pyrimidin-4-yl)-6-chloro-2,8-difluoroquinazolin-4-yl)-3,8-diazabicyclo[3.2.1]octane-8-carboxylate

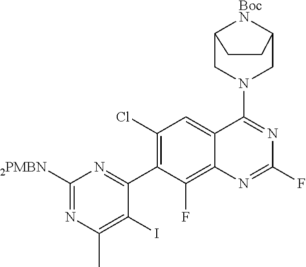

A mixture of tert-butyl 3-(7-(2-(bis(4-methoxybenzyl)amino)-6-methyl pyrimidin-4-yl)-6-chloro-2,8-difluoroquinazolin-4-yl)-3,8-diazabicyclo[3.2.1]octane-8-carboxylate (400 mg, 0.53 mmol), Ag₂SO₄ (370 mg, 1.2 mmol), I₂ (310 mg, 1.2 mmol) in DMF (10 mL) was stirred for 1 hour at room temperature. After completion, the resulting mixture was directly poured into water and extracted with EtOAc. Then the crude product was further purified by silica column to give the title product (300 mg, 0.34 mmol). MS (ESI, m/e) [M+1]⁺884.2

### Step 4: tert-butyl 3-(7-(2-(bis(4-methoxybenzyl)amino)-6-methyl-5-(trifluoro methyl)pyrimidin-4-yl)-6-chloro-2,8-difluoroquinazolin-4-yl)-3,8-diazabicyclo[3.2.1]octane-8-carboxylate

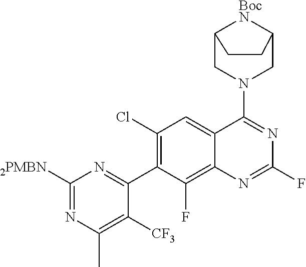

A mixture of tert-butyl 3-(7-(2-(bis(4-methoxybenzyl)amino)-5-iodo-6-methyl pyrimidin-4-yl)-6-chloro-2,8-difluoroquinazolin-4-yl)-3,8-diazabicyclo[3.2.1]octane-8-carboxylate (88.3 mg, 0.1 mmol), CuI (95 mg, 0.5 mmol), methyl 2,2-difluoro-2-(fluoro sulfonyl)acetate (192 mg, 1 mmol) in DMA (10 mL) was stirred for 2 hours at 90° C. After completion, the resulting mixture was directly poured into water and extracted with EtOAc. Then the crude product was further purified by silica column to give the title product (70 mg, 0.08 mmol). MS (ESI, m/e) [M+H]⁺ 826.3.

### Step 5: tert-butyl 3-(7-(2-(bis(4-methoxybenzyl)amino)-6-methyl-5-(trifluoro methyl)pyrimidin-4-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

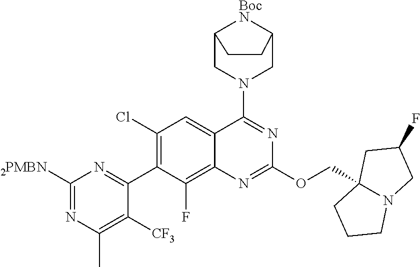

To a solution of ((2S,4R)-4-fluoro-1-methylpyrrolidin-2-yl)methanol (48 mg, 0.3 mmol) in THF (10 mL) was added NaH (24.0 mg, 0.6 mmol) and the mixture was stirred for 30 mins at room temperature. Then, tert-butyl 3-(7-(2-(bis(4-methoxybenzyl)amino)-6-methyl-5-(trifluoromethyl)pyrimidin-4-yl)-6-chloro-2,8-difluoroquinazolin-4-yl)-3,8-diazabicyclo [3.2.1]octane-8-carboxylate (70 mg, 0.08 mmol) was added. The reaction mixture was stirred for 2 hours at room temperature. After completion, the reaction was quenched by H₂O. Solvents were evaporated and the crude product was purified by silica column (eluting with DCM/MeOH=10/1) to give the title product (27 mg, 0.03 mmol). MS (ESI, m/e) [M+1]⁺965.4.

### Step 6: 4-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-6-methyl-5-(trifluoro methyl) pyrimidin-2-amine

A mixture of tert-butyl 3-(7-(2-(bis(4-methoxybenzyl)amino)-6-methyl-5-(trifluoromethyl)pyrimidin-4-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (27 mg, 0.03 mmol) in TFA (4 mL) was stirred for 8 hours at 40° C. After completion, the resulting mixture was directly concentrated and the residue was adjusted to pH=9 with DIPEA. Then the residues were purified by C18 column (eluting with H₂O/CH₃CN=3/1) to afford the title product (7.46 mg). ¹H NMR (500 MHz, DMSO-d₆) δ 7.87 (s, 1H), 7.71-7.62 (m, 2H), 5.33-5.22 (m, 1H), 4.40-4.37 (m, 2H), 4.29-4.27 (m, 2H), 4.01-3.97 (m, 2H), 3.55-3.52 (m, 3H), 3.14-3.02 (m, 3H), 2.83-2.81 (m, 1H), 2.06-2.00 (m, 3H), 1.86-1.77 (m, 7H). MS (ESI, m/e) [M+H]⁺ 625.4.

### Example 113: 6-(4-(3,8-diazabicyclo[3.2.1]octan-8-yl)-6-chloro-8-fluoro-2-(((2R,7aS)-2-fluorotetrahydro-1H-pyrrolizin-7a(5H)-yl)methoxy)quinazolin-7-yl)-4-ethynyl-5-(trifluoromethyl)pyridin-2-amine

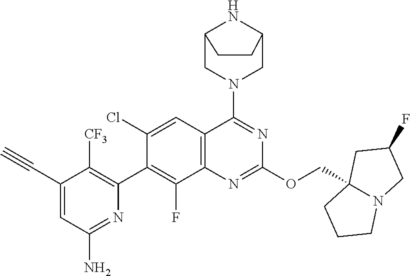 [SMILES: C#Cc1cc(N)nc(-c2c(Cl)cc3c(N4CC5CCC(C4)N5)nc(OC[C@@]45CCCN4C[C@H](F)C5)nc3c2F)c1C(F)(F)F]

Synthetic Route

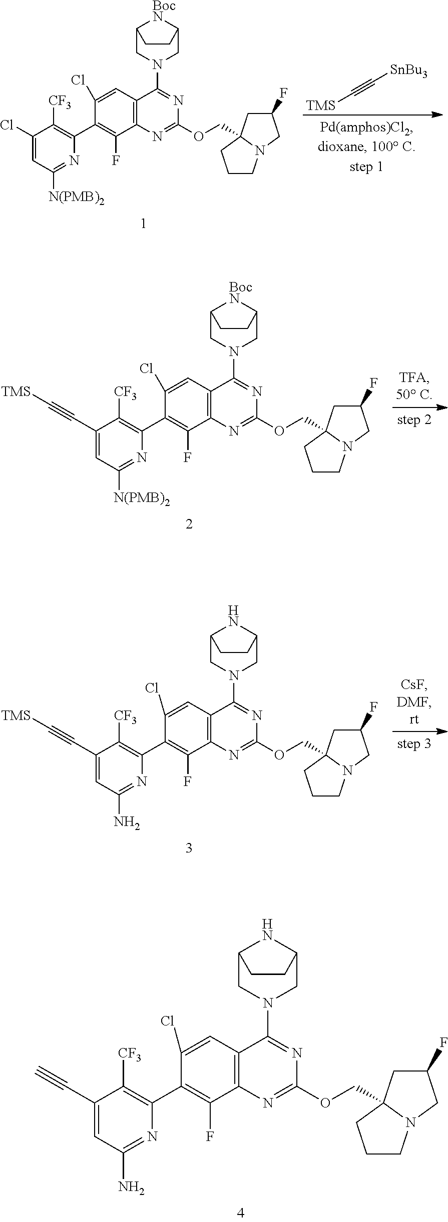

### Step 1: tert-butyl 8-(7-(6-(bis(4-methoxybenzyl)amino)-3-(trifluoromethyl)-4-((trimethylsilyl)ethynyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-3-carboxylate

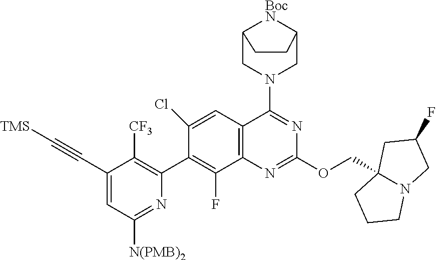

Into a 25 mL round-bottom flask were placed tert-butyl 8-(7-(6-(bis(4-methoxybenzyl)amino)-4-chloro-3-(trifluoro methyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-3-carboxylate (0.30 g, 0.31 mmol, 1.00 equiv), dioxane (3 mL), trimethyl((tributylstannyl)ethynyl)silane (0.35 g, 0.91 mmol, 3.00 equiv), Pd(amphos)Cl₂ (0.043 g, 0.06 mmol, 0.2 equiv) at rt. The resulting solution was stirred for 3 h at 100° C., then was extracted with 100 mL of ethyl acetate. concentrated under vacuum. The crude product was purified by TLC, mobile phase, PE:EA=1:2. Detector, 254/280 nm. This resulted in 0.31 g (97. % yield) of the title product. MS (ESI, m/e) [M+H]⁺ 1046/1048.

### Step 2: 6-(4-(3,8-diazabicyclo[3.2.1]octan-8-yl)-6-chloro-8-fluoro-2-(((2R,7aS)-2-fluorotetrahydro-1H-pyrrolizin-7a(5H)-yl)methoxy)quinazolin-7-yl)-5-(trifluoromethyl)-4-((trimethylsilyl)ethynyl)pyridin-2-amine

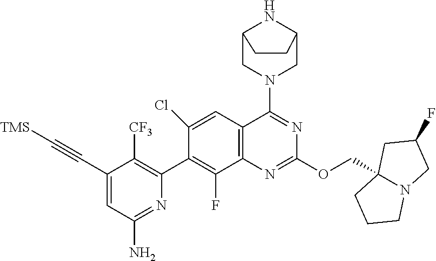 [SMILES: C[Si](C)(C)C#Cc1cc(N)nc(-c2c(Cl)cc3c(N4CC5CCC(C4)N5)nc(OC[C@@]45CCCN4C[C@H](F)C5)nc3c2F)c1C(F)(F)F]

Into a 25 mL round-bottom flask, were placed tert-butyl 8-(7-(6-(bis(4-methoxy benzyl)amino)-3-(trifluoromethyl)-4-((trimethylsilyl)ethynyl)pyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diaza bicyclo[3.2.1]octane-3-carboxylate (0.03 g, 0.29 mmol, 1.00 equiv), TFA (3 mL). The resulting solution was stirred for 2 h at 50° C. The resulting solution was basified to pH=8 with Na₂CO₃ solution, stirred for 30 min at rt, extracted with 100 mL of ethyl acetate and concentrated under vacuum. The crude product was purified by TLC, mobile phase, DCM:MeOH=10:1; Detector, 254/280 nm. This resulted in 0.1 g (49.5% yield) of the title product. MS (ESI, m/e) [M+H]⁺ 706/708.

### Step 3: 6-(4-(3,8-diazabicyclo[3.2.1]octan-8-yl)-6-chloro-8-fluoro-2-(((2R,7aS)-2-fluorotetrahydro-1H-pyrrolizin-7a(5H)-yl)methoxy)quinazolin-7-yl)-4-ethynyl-5-(trifluoro methyl)pyridin-2-amine

Into a 25 mL round-bottom flask, was placed 6-(4-(3,8-diazabicyclo[3.2.1]octan-8-yl)-6-chloro-8-fluoro-2-(((2R,7aS)-2-fluorotetrahydro-1H-pyrrolizin-7a(5H)-yl)methoxy) quinazolin-7-yl)-5-(trifluoromethyl)-4-((trimethylsilyl)ethynyl)pyridin-2-amine (0.1 g, 0.14 mmol, 1.00 equiv), DMF (2 mL), CsF (0.043 g, 0.28 mmol, 2.0 equiv). The resulting solution was stirred at rt for 1 h. Then, the solution was extracted with 100 mL of ethyl acetate, concentrated under vacuum, purified by Prep-HPLC to give the title product (13.4 mg, 14.92% yield). 1H NMR (400 MHz, CD₃OD) δ 8.51 (s, 1H), 7.90 (s, 1H), 6.87 (s, 1H), 5.55-5.28 (m, 1H), 4.98 (s, 2H), 4.50-4.30 (m, 2H), 4.09 (s, 1H), 3.70-3.41 (m, 4H), 3.23-3.15 (m, 2H), 2.99 (d, J=12.8 Hz, 2H), 2.6-2.0 (m, 10H). MS (ESI, m/e) [M+H]⁺ 634.05.

### Example 114: 3-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-5-methyl-4-(trifluoromethyl)phenol

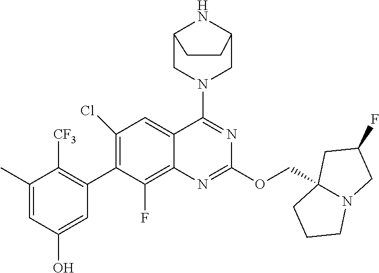 [SMILES: Cc1cc(O)cc(-c2c(Cl)cc3c(N4CC5CCC(C4)N5)nc(OC[C@@]45CCCN4C[C@H](F)C5)nc3c2F)c1C(F)(F)F]

Synthetic Route

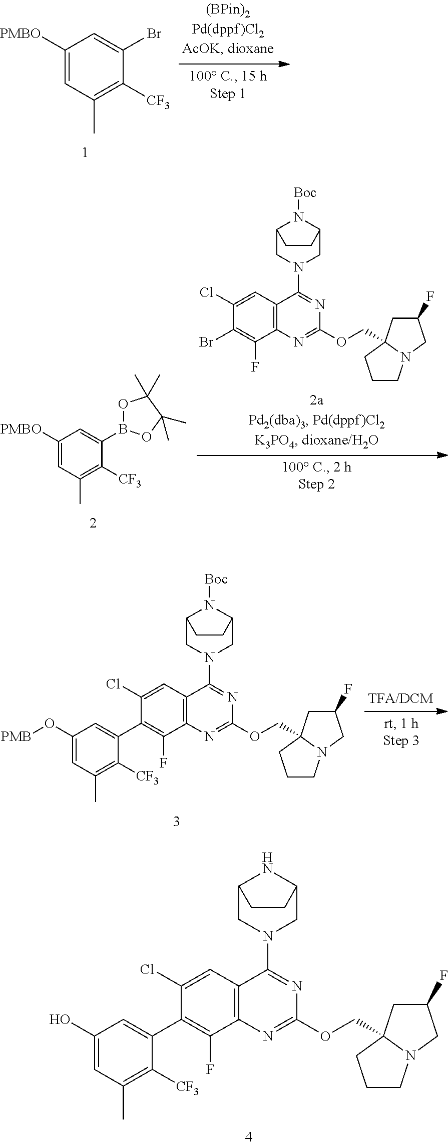

### Step 1: 2-(5-((4-methoxybenzyl)oxy)-3-methyl-2-(trifluoromethyl)phenyl)-4,4,5,5-tetramethyl-1,3,2-dioxaborolane

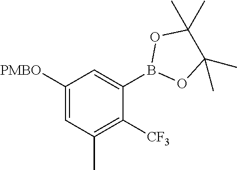

A mixture of 1-bromo-5-((4-methoxybenzyl)oxy)-3-methyl-2-(trifluoromethyl) benzene (197 mg, 0.53 mmol), 4,4,4′,4′,5,5,5′,5′-octamethyl-2,2′-bi(1,3,2-dioxaborolane) (267 mg, 1.05 mmol), Pd(dppf)Cl₂ (45 mg, 0.06 mmol) and AcOK (156 mg, 1.59 mmol) in dioxane (15 mL) was stirred at 100° C. for 16 hrs. The reaction mixture was filtered with a pad of celite and the filtrate was concentrated and purified by Prep-TLC (PE:EA=10:1) to give the title product (108 mg, 48% yield). ¹H NMR (500 MHz, CDCl₃) δ 7.33 (d, J=8.6 Hz, 2H), 6.91 (d, J=8.6 Hz, 2H), 6.86 (s, 1H), 6.83 (s, 1H), 4.99 (s, 2H), 3.82 (s, 3H), 2.43 (s, 3H), 1.36 (s, 12H).

### Step 2: tert-butyl 3-(6-chloro-8-fluoro-2-(((2R,7aS)-2-fluorotetrahydro-1H-pyrrolizin-7a(5H)-yl)methoxy)-7-(5-((4-methoxybenzyl)oxy)-3-methyl-2-(trifluoromethyl) phenyl)quinazolin-4-yl)-3,8-diazabicyclo [3.2.1]octane-8-carboxylate

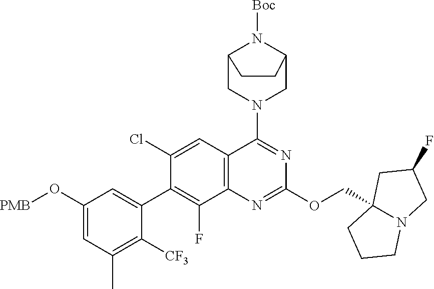

To a mixture of 2-(5-((4-methoxybenzyl)oxy)-3-methyl-2-(trifluoromethyl) phenyl)-4,4,5,5-tetramethyl-1,3,2-dioxaborolane (108 mg, 0.26 mmol), tert-butyl 3-(7-bromo-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (107 mg, 0.17 mmol), Pd₂(dba)₃ (45 mg, 0.05 mmol), Pd(dbpf)Cl₂.DCM (45 mg, 0.06 mmol), AntPhos (45 mg, 0.1 mmol) and K₃PO₄ (108 mg, 0.51 mmol) was added dioxane (25 mL) and H₂O (5 mL). The resulting mixture was stirred at 100° C. for 2 hrs. After completion, the reaction mixture was filtered via a pad of celite and the filtrate was concentrated to give the residue. The residue was purified by combi-flash (DCM/MeOH=10/1) to give the title product (146 mg, 48% yield). MS (ESI, m/e) [M+H]⁺ 844.5.

### Step 3: 3-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-5-methyl-4-(trifluoro methyl)phenol

A solution of tert-butyl 3-(6-chloro-8-fluoro-2-(((2R,7aS)-2-fluorotetrahydro-1H-pyrrolizin-7a(5H)-yl)methoxy)-7-(5-((4-methoxybenzyl)oxy)-3-methyl-2-(trifluoromethyl) phenyl)quinazolin-4-yl)-3,8-diazabicyclo[3.2.1]octane-8-carboxylate (100 mg, 0.12 mmol) in TFA (6 mL) was stirred at room temperature for 2 hrs, the resulting solution was concentrated and treated with NH₃/MeOH (7M, 10 mL). Then, the solution was concentrated to give a residue. The residue was purified by Prep-HPLC to give the title product (50 mg, 67% yield). ¹H NMR (500 MHz, CD₃OD) δ 7.91 (s, 1H), 6.90 (s, 1H), 6.45 (s, 1H), 5.57-5.46 (m, 1H), 4.69-4.55 (m, 5H), 4.12 (s, 2H), 3.89-3.71 (m, 5H), 2.65-2.47 (m, 2H), 2.39-2.35 (m, 1H), 2.30-2.24 (m, 2H), 2.15-2.03 (m, 5H). MS (ESI, m/e) [M+H]⁺ 628.4.

### Example 115: 8-((2S)-2-((7-(2-amino-3-cyano-7-fluorobenzo[b]thiophen-4-yl)-4-(3,8-diazabicyclo[3.2.1]octan-3-yl)-6-chloro-8-fluoroquinazolin-2-yl)oxy)propyl)-8-azabicyclo [3.2.1]octane-3-carbonitrile

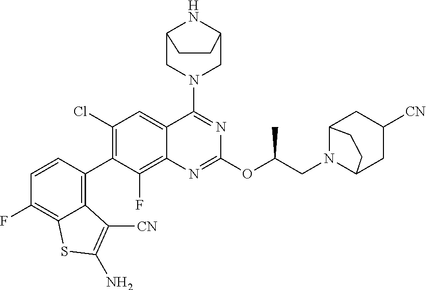 [SMILES: C[C@@H](CN1C2CCC1CC(C#N)C2)Oc1nc(N2CC3CCC(C2)N3)c2cc(Cl)c(-c3ccc(F)c4sc(N)c(C#N)c34)c(F)c2n1]

Synthetic Route

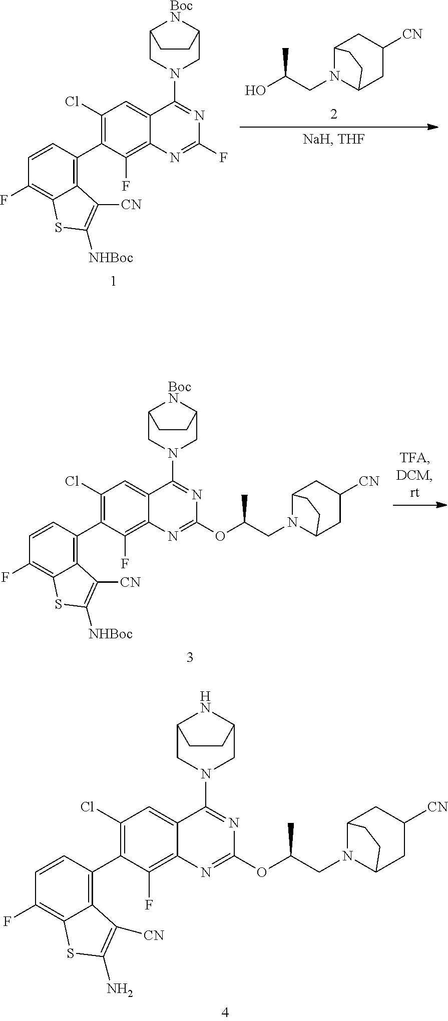 [SMILES: CC(C)(C)OC(=O)Nc1sc2c(F)ccc(-c3c(Cl)cc4c(N5CC6CCC(C5)N6C(=O)OC(C)(C)C)nc(F)nc4c3F)c2c1C#N ; C[C@H](O)CN1C2CCC1CC(C#N)C2 ; C[C@@H](CN1C2CCC1CC(C#N)C2)Oc1nc(N2CC3CCC(C2)N3C(=O)OC(C)(C)C)c2cc(Cl)c(-c3ccc(F)c4sc(NC(=O)OC(C)(C)C)c(C#N)c34)c(F)c2n1 ; C[C@@H](CN1C2CCC1CC(C#N)C2)Oc1nc(N2CC3CCC(C2)N3)c2cc(Cl)c(-c3ccc(F)c4sc(N)c(C#N)c34)c(F)c2n1]

### Step 1: tert-butyl 3-(7-(2-((tert-butoxycarbonyl)amino)-3-cyano-7-fluorobenzo [b]thiophen-4-yl)-6-chloro-2-(((2S)-1-(3-cyano-8-azabicyclo[3.2.1]octan-8-yl)propan-2-yl)oxy)-8-fluoroquinazolin-4-yl)-3,8-diazabicyclo[3.2.1]octane-8-carboxylate

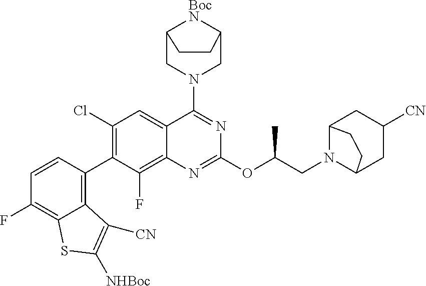 [SMILES: C[C@@H](CN1C2CCC1CC(C#N)C2)Oc1nc(N2CC3CCC(C2)N3C(=O)OC(C)(C)C)c2cc(Cl)c(-c3ccc(F)c4sc(NC(=O)OC(C)(C)C)c(C#N)c34)c(F)c2n1]

To a solution of (S)-1-(4-fluoropiperidin-1-yl)propan-2-ol (58 mg, 0.3 mmol) and NaH (7.2 mg, 0.3 mmol) in THF (2 mL) was added tert-butyl 3-(7-(2-((tert-butoxycarbonyl) amino)-3-cyano-7-fluorobenzo[b]thiophen-4-yl)-6-chloro-2,8-difluoroquinazolin-4-yl)-3,8-diazabicyclo[3.2.1]octane-8-carboxylate (70 mg, 0.1 mmol) at 0° C. The resulting mixture was stirred at room temperature for 2 hrs. After completion, the reaction mixture was added water, extracted with EA. The combined organic layers were dried over Na₂SO₄, filtered, concentrated and the residue was purified by Prep-TLC (PE:EA=1:1) to give the title product (44 mg, 50.3%), MS (ESI, m/e) [M+1]⁺875.5.

### Step 2: 8-((2S)-2-((7-(2-amino-3-cyano-7-fluorobenzo[b]thiophen-4-yl)-4-(3,8-diazabicyclo[3.2.1]octan-3-yl)-6-chloro-8-fluoroquinazolin-2-yl)oxy)propyl)-8-azabicyclo [3.2.1]octane-3-carbonitrile

To a solution of tert-butyl 3-(7-(2-((tert-butoxycarbonyl)amino)-3-cyano-7-fluoro benzo[b]thiophen-4-yl)-6-chloro-2-(((2S)-1-(3-cyano-8-azabicyclo[3.2.1]octan-8-yl)propan-2-yl) oxy)-8-fluoroquinazolin-4-yl)-3,8-diazabicyclo[3.2.1]octane-8-carboxylate (44 mg, 0.05 mmol) in DCM (4 mL) was added TFA (2 mL) at room temperature. The resulting mixture was stirred at room temperature for 3 hrs. After completion, the reaction mixture was concentrated and the residue was purified by prep-HPLC to give the title product (8.5 mg, FA salt, 23.6%). ¹H NMR (500 MHz, CD₃OD) δ 8.37 (s, 1H), 7.88 (s, 1H), 7.22-7.18 (m, 1H), 7.06-7.03 (m, 1H), 5.61-5.51 (m, 1H), 4.69-4.47 (m, 2H), 4.15-4.09 (m, 2H), 3.85-3.54 (m, 4H), 3.02-2.65 (m, 3H), 2.24-1.65 (m, 12H), 1.44 (d, J=6.1 Hz, 3H). MS (ESI, m/e) [M+H]⁺ 675.5.

### Example 116: 4-(4-(3,8-diazabicyclo[3.2.1]octan-3-yl)-6-chloro-8-fluoro-2-(((S)-1-(4-fluoropiperidin-1-yl)propan-2-yl)oxy)quinazolin-7-yl)-2-amino-7-fluorobenzo[b]thiophene-3-carbonitrile

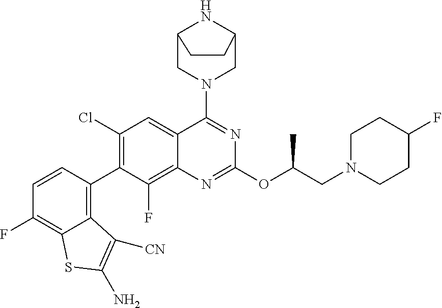 [SMILES: C[C@@H](CN1CCC(F)CC1)Oc1nc(N2CC3CCC(C2)N3)c2cc(Cl)c(-c3ccc(F)c4sc(N)c(C#N)c34)c(F)c2n1]

Synthetic Route

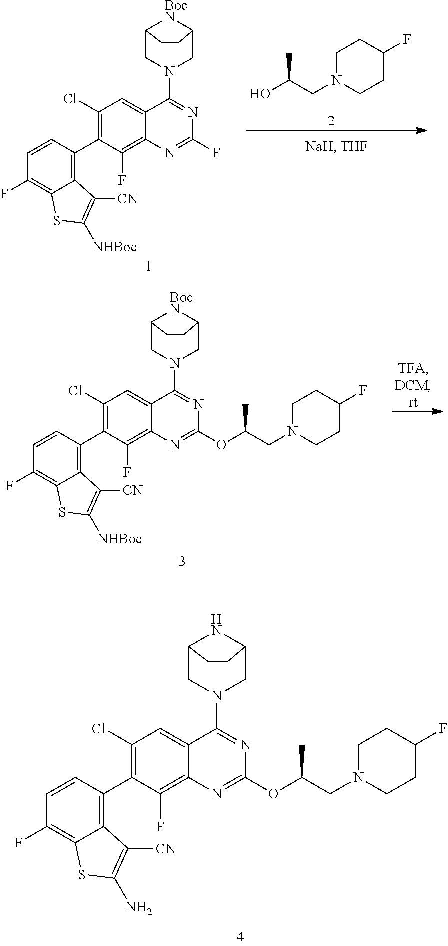 [SMILES: CC(C)(C)OC(=O)Nc1sc2c(F)ccc(-c3c(Cl)cc4c(N5CC6CCC(C5)N6C(=O)OC(C)(C)C)nc(F)nc4c3F)c2c1C#N ; C[C@H](O)CN1CCC(F)CC1 ; C[C@@H](CN1CCC(F)CC1)Oc1nc(N2CC3CCC(C2)N3C(=O)OC(C)(C)C)c2cc(Cl)c(-c3ccc(F)c4sc(NC(=O)OC(C)(C)C)c(C#N)c34)c(F)c2n1 ; C[C@@H](CN1CCC(F)CC1)Oc1nc(N2CC3CCC(C2)N3)c2cc(Cl)c(-c3ccc(F)c4sc(N)c(C#N)c34)c(F)c2n1]

### Step 1: tert-butyl 3-(7-(2-((tert-butoxycarbonyl)amino)-3-cyano-7-fluorobenzo[b]thiophen-4-yl)-6-chloro-8-fluoro-2-(((S)-1-(4-fluoropiperidin-1-yl)propan-2-yl)oxy)quinazolin-4-yl)-3,8-diazabicyclo[3.2.1]octane-8-carboxylate

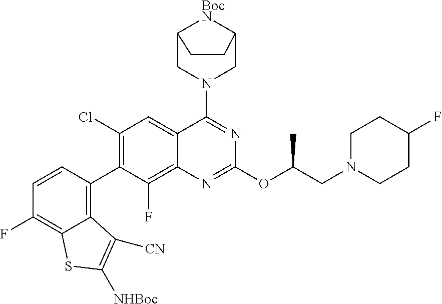 [SMILES: C[C@@H](CN1CCC(F)CC1)Oc1nc(N2CC3CCC(C2)N3C(=O)OC(C)(C)C)c2cc(Cl)c(-c3ccc(F)c4sc(NC(=O)OC(C)(C)C)c(C#N)c34)c(F)c2n1]

To a solution of (S)-1-(4-fluoropiperidin-1-yl)propan-2-ol (49 mg, 0.3 mmol) and NaH (7.2 mg, 0.3 mmol) in THF (2 mL) was added tert-butyl 3-(7-(2-((tert-butoxycarbonyl) amino)-3-cyano-7-fluorobenzo[b]thiophen-4-yl)-6-chloro-2,8-difluoroquinazolin-4-yl)-3,8-diaza bicyclo[3.2.1]octane-8-carboxylate (70 mg, 0.1 mmol) at 0° C. The resulting mixture was stirred at room temperature for 2 hrs. After completion, the reaction mixture was added water, extracted with EA. Then, the combined organic layers were dried over Na₂SO₄, filtered, concentrated and the residue was purified by Prep-TLC (PE:EA=1:1) to give the title product (42 mg, 50%), MS (ESI, m/e) [M+1]⁺842.

### Step 2: 4-(4-(3,8-diazabicyclo[3.2.1]octan-3-yl)-6-chloro-8-fluoro-2-(((S)-1-(4-fluoropiperidin-1-yl)propan-2-yl)oxy)quinazolin-7-yl)-2-amino-7-fluorobenzo[b]thiophene-3-carbonitrile

To a solution of tert-butyl 3-(7-(2-((tert-butoxycarbonyl)amino)-3-cyano-7-fluorobenzo[b]thiophen-4-yl)-6-chloro-8-fluoro-2-(((S)-1-(4-fluoropiperidin-1-yl)propan-2-yl)oxy)quinazolin-4-yl)-3,8-diazabicyclo[3.2.1]octane-8-carboxylate (42 mg, 0.05 mmol) in DCM (2 mL) was added TFA (2 mL) at room temperature. The resulting mixture was stirred at room temperature for 3 hrs. After completion, the reaction mixture was concentrated and the residue was purified by prep-HPLC to give the title product (6.0 mg, 18.8%). ¹H NMR (500 MHz, CD₃OD) δ 7.90 (s, 1H), 7.22-7.20 (m, 1H), 7.07-7.03 (m, 1H), 5.72-5.64 (m, 1H), 4.82-4.47 (m, 4H), 4.19-4.13 (m, 2H), 3.82-3.75 (m, 2H), 3.31-2.95 (m, 5H), 2.14-1.91 (m, 8H), 1.44 (d, J=6.3 Hz, 3H). MS (ESI, m/e) [M+1]⁺642.4.

### Example 117: 6-(4-(3,8-diazabicyclo[3.2.1]octan-3-yl)-6-chloro-2-(((2R,7aS)-2-fluorotetrahydro-1H-pyrrolizin-7a(5H)-yl)methoxy)quinazolin-7-yl)-4-methyl-5-(trifluoro methyl)pyridin-2-amine

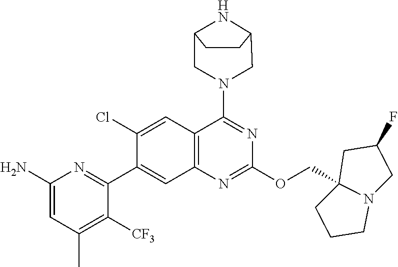 [SMILES: Cc1cc(N)nc(-c2cc3nc(OC[C@@]45CCCN4C[C@H](F)C5)nc(N4CC5CCC(C4)N5)c3cc2Cl)c1C(F)(F)F]

Synthetic Route

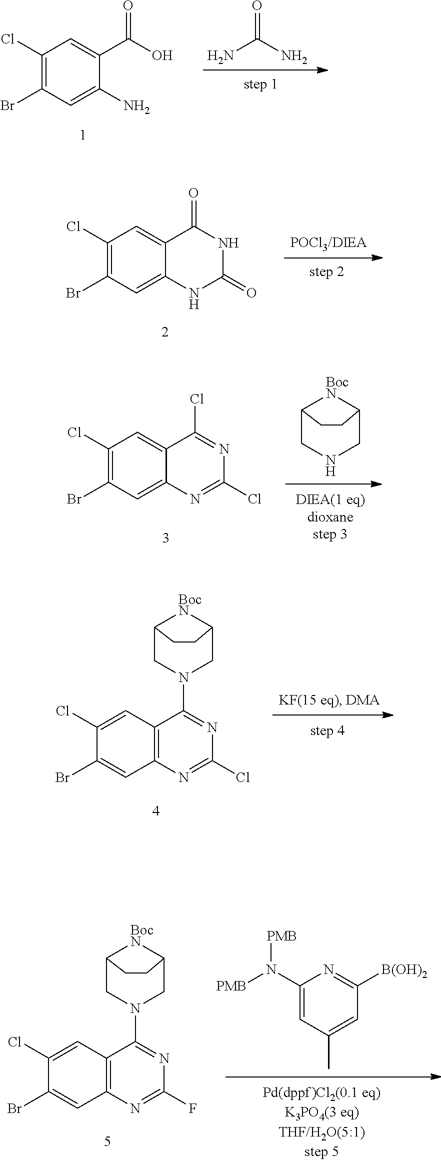

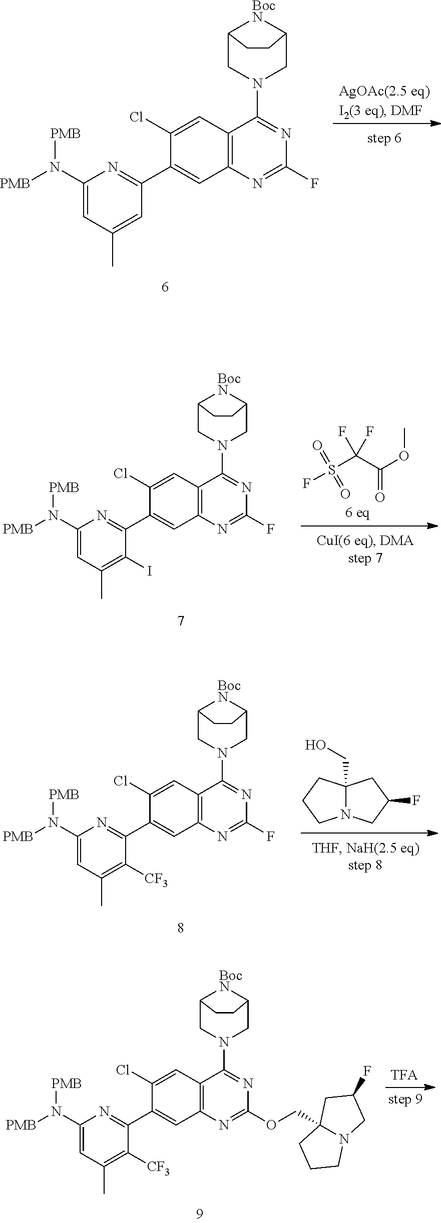

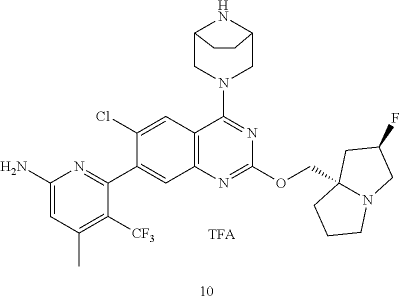 [SMILES: Cc1cc(N)nc(-c2cc3nc(OC[C@@]45CCCN4C[C@H](F)C5)nc(N4CC5CCC(C4)N5)c3cc2Cl)c1C(F)(F)F]

### Step 1: 7-bromo-6-chloroquinazoline-24(1H,3H)-dione

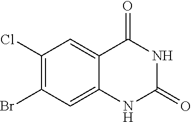 [SMILES: O=c1[nH]c(=O)c2cc(Cl)c(Br)cc2[nH]1]

A mixture of 2-amino-4-bromo-5-chlorobenzoic acid (10 g, 39.92 mmol) and urea (47.95 g, 798.5 mmol) was stirred overnight at 140° C. The hot mixture was poured into H₂O and filtered to give the solid 7-bromo-6-chloro-1,3-dihydroquinazoline-2,4-dione (11 g, crude).

### Step 2: 7-bromo-2,4,6-trichloroquinazoline

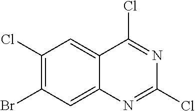 [SMILES: Clc1nc(Cl)c2cc(Cl)c(Br)cc2n1]

A solution of 7-bromo-6-chloro-1,3-dihydroquinazoline-2,4-dione (11 g, 39.93 mmol) in POCl₃ (110 mL) and DIEA (11 mL) was stirred overnight at 110° C. The resulting solution was concentrated and purified by silica gel column to give the title 7-bromo-2,4,6-trichloroquinazoline (7 g, 56%).

### Step 3: tert-butyl 3-(7-bromo-2,6-dichloroquinazolin-4-yl)-3,8-diazabicyclo [3.2.1]octane-8-carboxylate

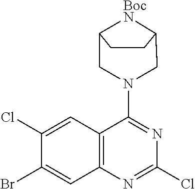 [SMILES: CC(C)(C)OC(=O)N1C2CCC1CN(c1nc(Cl)nc3cc(Br)c(Cl)cc13)C2]

To a solution of 7-bromo-2,4,6-trichloroquinazoline (7 g, 22.41 mmol) and tert-butyl 3,8-diazabicyclo[3.2.1]octane-8-carboxylate (4.76 g, 22.41 mmol) in dioxane (80 mL) was added DEIA (2.89 g, 22.41 mmol) at 0° C. After stirring 1 h at room temperature, the resulting solution was concentrated and purified by silica gel column to give the title product (9 g, 82%).

### Step 4: tert-butyl 3-(7-bromo-6-chloro-2-fluoroquinazolin-4-yl)-3,8-diazabicyclo [3.2.1]octane-8-carboxylate

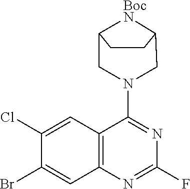 [SMILES: CC(C)(C)OC(=O)N1C2CCC1CN(c1nc(F)nc3cc(Br)c(Cl)cc13)C2]

To a solution of tert-butyl 3-(7-bromo-2,6-dichloroquinazolin-4-yl)-3,8-diazabicyclo[3.2.1] octane-8-carboxylate (1 g, 2.05 mmol) in DMA (15 mL) was added KF (1.78 g, 30.72 mmol) at room temperature. After stirring overnight at 140° C., the resulting solution was quenched with H₂O at 0° C. and extracted with EA. The organic layer was concentrated and purified by silica gel column to give the title product (600 mg, 62%). MS (ESI, m/e) [M+H]⁺ 471.05/473.05.

### Step 5: tert-butyl 3-(7-(6-(bis(4-methoxybenzyl)amino)-4-methylpyridin-2-yl)-6-chloro-2-fluoroquinazolin-4-yl)-3,8-diazabicyclo[3.2.1]octane-8-carboxylate

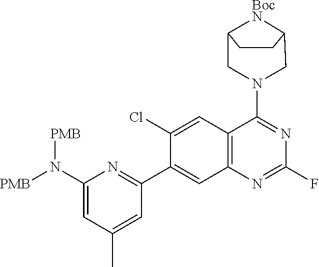

To a solution of tert-butyl 3-(7-bromo-6-chloro-2-fluoroquinazolin-4-yl)-3,8-diazabicyclo[3.2.1]octane-8-carboxylate (600 mg, 1.27 mmol) in THF (15 mL) and H₂O (3 mL) was added 6-(bis((4-methoxyphenyl)methyl)amino)-4-methylpyridin-2-ylboronic acid (1.50 g, 3.82 mmol), Pd(PPh₃)₄ (147 mg, 0.13 mmol) and K₃PO₄ (0.81 g, 3.82 mmol) at room temperature. After stirring overnight at 65° C. for 3 h, the resulting solution was extracted with EA. The organic layer was concentrated and purified by silica gel column to give the title product (630 mg, 67%). MS (ESI, m/e) [M+H]⁺ 739.35.

### Step 6: tert-butyl 3-(7-(6-(bis(4-methoxybenzyl)amino)-3-iodo-4-methylpyridin-2-yl)-6-chloro-2-fluoroquinazolin-4-yl)-3,8-diazabicyclo[3.2.1]octane-8-carboxylate

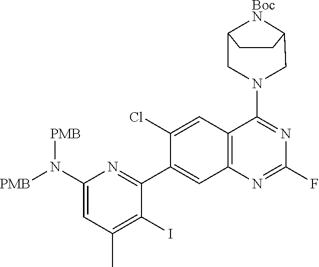

To a solution of tert-butyl 3-(7-(6-(bis((4-methoxyphenyl)methyl)amino)-4-methylpyridin-2-yl)-6-chloro-2-fluoroquinazolin-4-yl)-3,8-diazabicyclo[3.2.1]octane-8-carboxylate (250 mg, 0.35 mmol) in DMF (5 mL) was added silver acetate (140.3 mg, 0.85 mmol) and I₂ (257.5 mg, 1.01 mmol) at 0° C. The mixture was stirred for 4 h at room temperature. The resulting mixture was diluted with H₂O at 0° C. The aqueous layer was extracted with EA. The organic layer was concentrated. The residue was applied onto a silica gel column with ethyl acetate/hexane (0:100-20:80) to give the title product (220 mg, 75%). MS (ESI, m/e) [M+H]⁺ 865.40.

### Step 7: tert-butyl 3-(7-(6-(bis(4-methoxybenzyl)amino)-4-methyl-3-(trifluoro methyl)pyridin-2-yl)-6-chloro-2-fluoroquinazolin-4-yl)-3,8-diazabicyclo[3.2.1]octane-8-carboxylate

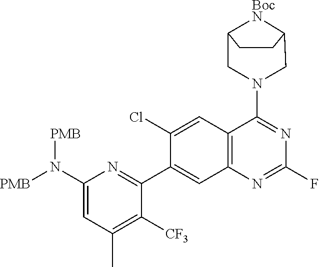

To a solution of tert-butyl 3-(7-(6-(bis((4-methoxyphenyl)methyl)amino)-3-iodo-4-methylpyridin-2-yl)-6-chloro-2-fluoroquinazolin-4-yl)-3,8-diazabicyclo[3.2.1]octane-8-carboxylate (200 mg, 0.23 mmol) in DMA (5 mL) was added methyl 2,2-difluoro-2-sulfoacetate (266.4 mg, 1.37 mmol) and CuI (264.1 mg, 1.37 mmol) at room temperature. The mixture was stirred for 2 h at 90° C. The resulting mixture was filtered, the filtrate was diluted with H₂O at 0° C. The aqueous layer was extracted with EA. The organic layer was concentrated and applied onto a silica gel column to give the title product (100 mg, 53%). MS (ESI, m/e) [M+H]⁺ 807.50.

### Step 8: tert-butyl 3-(7-(6-(bis(4-methoxybenzyl)amino)-4-methyl-3-(trifluoro methyl)pyridin-2-yl)-6-chloro-2-(((2R,7aS)-2-fluorotetrahydro-1H-pyrrolizin-7a(5H)-yl) methoxy)quinazolin-4-yl)-3,8-diazabicyclo [3.2.1]octane-8-carboxylate

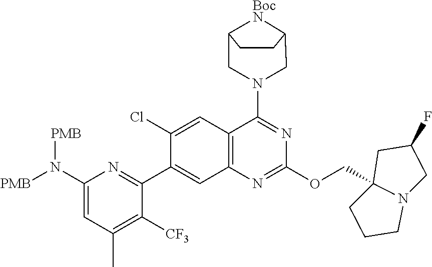

To a solution of ((2R,7aS)-2-fluoro-hexahydropyrrolizin-7a-yl)methanol (59.2 mg, 0.37 mmol) in THF (2 mL) was added NaH (12.4 mg, 0.310 mmol, 60% purity) at 0° C. The mixture was stirred for 1 h at room temperature. Tert-butyl 3-(7-(6-(bis((4-methoxy phenyl) methyl)amino)-4-methyl-3-(trifluoromethyl)pyridine-2-yl)-6-chloro-2-fluoroquinazolin-4-yl)-3,8-diazabicyclo [3.2.1]octane-8-carboxylate (100 mg, 0.12 mmol) was added and the mixture was stirred for 2 h at rt. The reaction mixture was quenched by water and extracted with EA. The organic layer was concentrated. The residue was purified by Prep-TLC (DCM:MeOH=10:1) to give the title product (70 mg, 59%). MS (ESI, m/e) [M+H]⁺ 946.45.

### Step 9: 6-(4-(3,8-diazabicyclo[3.2.1]octan-3-yl)-6-chloro-2-(((2R,7aS)-2-fluoro tetrahydro-1H-pyrrolizin-7a(5H)-yl)methoxy)quinazolin-7-yl)-4-methyl-5-(trifluoromethyl) pyridin-2-amine

A solution of tert-butyl 3-(2-(((2R,7aS)-2-fluoro-hexahydropyrrolizin-7a-yl) methoxy)-7-(6-(bis((4-methoxyphenyl)methyl)amino)-4-methyl-3-(trifluoromethyl)pyridin-2-yl)-6-chloroquinazolin-4-yl)-3,8-diazabicyclo[3.2.1]octane-8-carboxylate (35 mg, 0.04 mmol) in TFA (2 mL) was stirred for 2 h at 50° C. The resulting mixture was concentrated and purified by Prep-HPLC to give title product (9.4 mg, TFA salt, 35%). ¹H NMR (400 MHz, CD₃OD) δ 8.10 (s, 1H), 7.63 (s, 1H), 6.72 (s, 1H), 5.75-5.46 (m, 1H), 4.76-4.65 (m, 4H), 4.24 (s, 2H), 4.03-3.84 (m, 5H), 3.54-3.45 (m, 1H), 2.79-2.62 (m, 2H), 2.60-2.55 (m, 4H), 2.484-2.32 (m, 2H), 2.24-2.01 (m, 5H). MS (ESI, m/e) [M-TFA+H]⁺ 606.25.

### Example 118: 2-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-6-amino-3-iodo-4-methylbenzonitrile

Synthetic Route

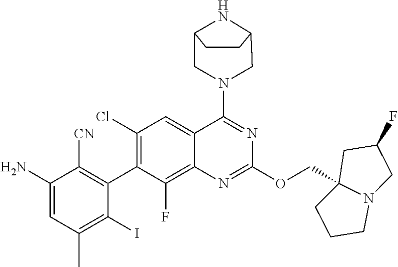 [SMILES: Cc1cc(N)c(C#N)c(-c2c(Cl)cc3c(N4CC5CCC(C4)N5)nc(OC[C@@]45CCCN4C[C@H](F)C5)nc3c2F)c1I]

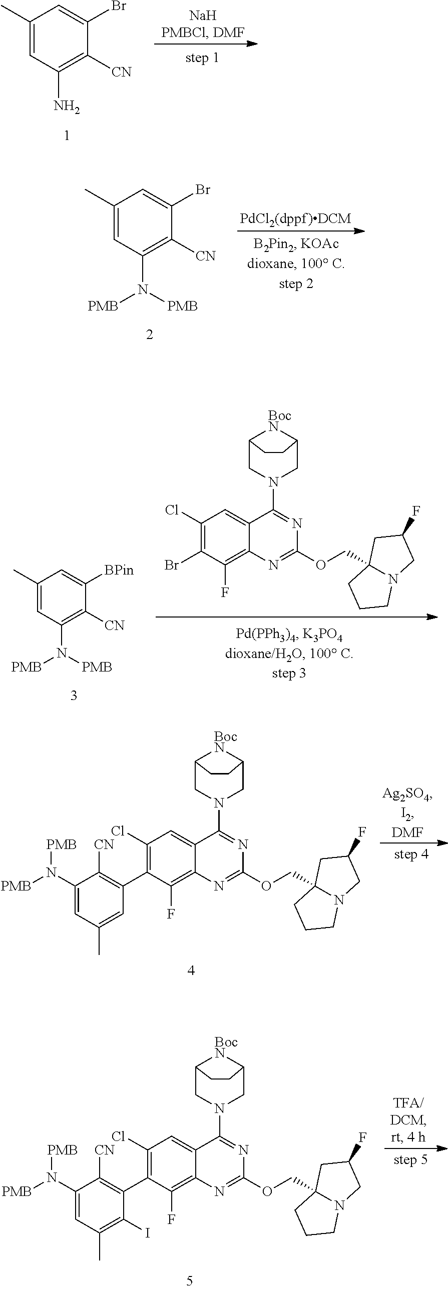

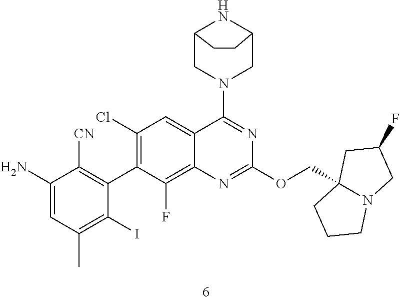 [SMILES: Cc1cc(N)c(C#N)c(-c2c(Cl)cc3c(N4CC5CCC(C4)N5)nc(OC[C@@]45CCCN4C[C@H](F)C5)nc3c2F)c1I]

### Step 1: 2-(bis(4-methoxybenzyl)amino)-6-bromo-4-methylbenzonitrile

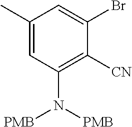

To a solution of 2-amino-6-bromo-4-methylbenzonitrile (1050 mg, 5.0 mmol), and NaH (252 mg, 10.5 mmol) in DMF (10 mL) was added dropwise PMBCl (1730 mg, 11 mmol) at 0° C. for 10 mins. The resulting mixture was stirred at room temperature for 2 hrs. After completion, the reaction mixture was quenched by water and extracted with EA. The combined organic layer was dried over Na₂SO₄, filtered and concentrated. The residue was purified by silica gel (PE:EA=3:1) to give the title product (1000 mg, 44.2%). MS (ESI, m/e) [M+1]⁺451, 453.

### Step 2: 2-(bis(4-methoxybenzyl)amino)-4-methyl-6-(4,4,5,5-tetramethyl-1,3,2-dioxaborolan-2-yl)benzonitrile

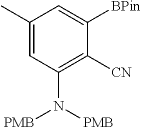

The mixture of 2-(bis(4-methoxybenzyl)amino)-6-bromo-4-methylbenzonitrile (225 mg, 0.5 mmol), B₂Pin₂ (254 mg, 1.0 mmol) and PdCl₂(dppf)DCM (41 mg, 0.05 mmol), KOAc (150 mg, 1.5 mmol) in dioxane (5 mL) was stirred at 100° C. for 15 hrs. After completion, the reaction mixture was purified by Prep-TLC (PE:EA=5:1) to give the titled product (202 mg, 81.1%). MS (ESI, m/e) [M+1]⁺499.

### Step 3: tert-butyl 3-(7-(3-(bis(4-methoxybenzyl)amino)-2-cyano-5-methylphenyl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

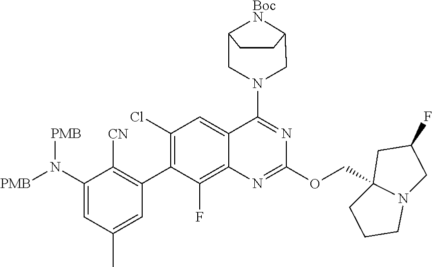

The mixture of tert-butyl 3-(7-bromo-6-chloro-8-fluoro-2-(((2R,7aS)-2-fluoro tetrahydro-1H-pyrrolizin-7a(5H)-yl)methoxy)quinazolin-4-yl)-3,8-diazabicyclo [3.2.1]octane-8-carboxylate (62.7 mg, 0.1 mmol), 2-(bis(4-methoxybenzyl)amino)-4-methyl-6-(4,4,5,5-tetra methyl-1,3,2-dioxaborolan-2-yl)benzonitrile (100 mg, 0.2 mmol), Pd(PPh₃)₄ (12 mg, 0.01 mmol) and K₃PO₄ (63 mg, 0.3 mmol) in dioxane (4 mL) and water (2 mL) was stirred at 100° C. for 2 hrs. After completion, the reaction mixture was purified by Prep-TLC (PE:EA=1:3) to give the titled product (40 mg, crude). MS (ESI, m/e) [M+1]⁺920.

### Step 4: tert-butyl 3-(7-(3-(bis(4-methoxybenzyl)amino)-2-cyano-6-iodo-5-methyl phenyl)-6-chloro-8-fluoro-2-(((2R,7aS)-2-fluorotetrahydro-1H-pyrrolizin-7a(5H)-yl)methoxy) quinazolin-4-yl)-3,8-diazabicyclo[3.2.1]octane-8-carboxylate

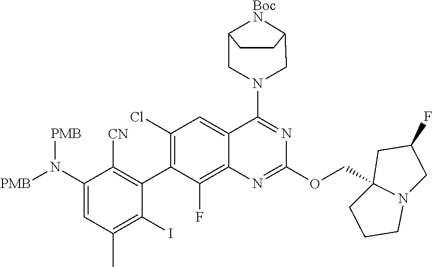

The mixture of tert-butyl 3-(7-(3-(bis(4-methoxybenzyl)amino)-2-cyano-5-methyl phenyl)-6-chloro-8-fluoro-2-(((2R,7aS)-2-fluorotetrahydro-1H-pyrrolizin-7a(5H)-yl)methoxy) quinazolin-4-yl)-3,8-diazabicyclo[3.2.1]octane-8-carboxylate (380 mg, 0.413 mmol), Ag₂SO₄ (65 mg, 0.207 mmol) and I₂ (105 mg, 0.413 mmol) in DMF (10 mL) was stirred at room temperature for 15 hrs. After completion, the reaction mixture was added water, extracted with EA. The combined organic layer was dried over Na₂SO₄, filtered, concentrated and the residue was purified by Prep-TLC (PE:EA=1:5) to give the title product (270 mg, crude). MS (ESI, m/e) [M+1]⁺1046.

### Step 5: 2-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-6-amino-3-iodo-4-methyl benzonitrile

To a solution of tert-butyl 3-(7-(3-(bis(4-methoxybenzyl)amino)-2-cyano-6-iodo-5-methylphenyl)-6-chloro-8-fluoro-2-(((2R,7aS)-2-fluorotetrahydro-1H-pyrrolizin-7a(5H)-yl) methoxy)quinazolin-4-yl)-3,8-diazabicyclo[3.2.1]octane-8-carboxylate (31.5 mg, 0.03 mmol) in DCM (4 mL) was added TFA (2 mL) at room temperature. The resulting mixture was stirred at room temperature for 3 hrs. After completion, the reaction mixture was concentrated and the residue was purified by prep-HPLC to give the title product (1.72 mg, FA salt, 7.5%). ¹H NMR (500 MHz, CD₃OD) δ 8.47 (s, 1H), 7.97 (s, 1H), 6.93 (s, 1H), 5.47-5.36 (m, 1H), 4.62-4.38 (m, 4H), 3.90 (s, 2H), 3.72-3.69 (m, 2H), 3.62-3.44 (m, 3H), 3.22-3.16 (m, 1H), 2.46 (s, 3H), 2.44-1.89 (m, 10H). MS (ESI, m/e) [M+1]⁺706.5.

### Example 119: 7-(6-amino-4-methyl-3-(trifluoromethyl)pyridin-2-yl)-4-(3,8-diazabicyclo [3.2.1]octan-3-yl)-8-fluoro-2-(((2R,7aS)-2-fluorotetrahydro-1H-pyrrolizin-7a(5H)-yl)methoxy) quinazoline-6-carbonitrile

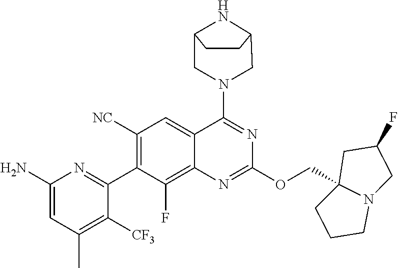 [SMILES: Cc1cc(N)nc(-c2c(C#N)cc3c(N4CC5CCC(C4)N5)nc(OC[C@@]45CCCN4C[C@H](F)C5)nc3c2F)c1C(F)(F)F]

Synthetic Route

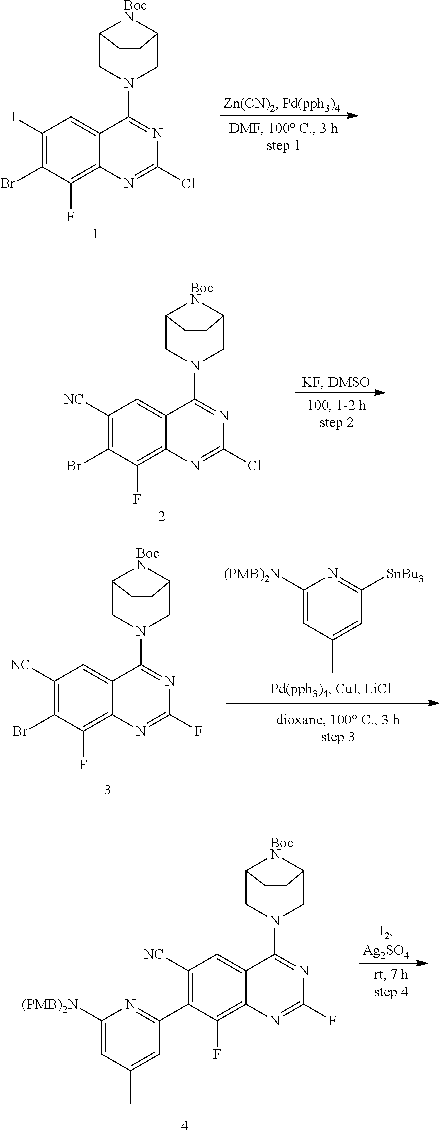

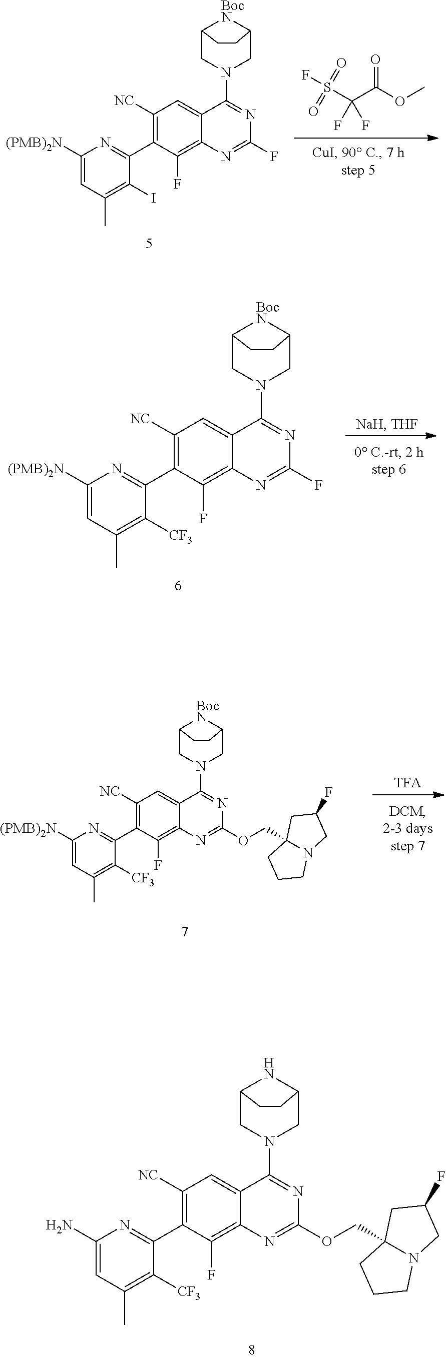

### Step 1: tert-butyl 3-(7-bromo-2-chloro-6-cyano-8-fluoroquinazolin-4-yl)-3,8-diazabicyclo[3.2.1]octane-8-carboxylate

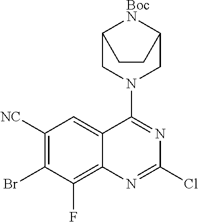 [SMILES: CC(C)(C)OC(=O)N1C2CCC1CN(c1nc(Cl)nc3c(F)c(Br)c(C#N)cc13)C2]

To a mixture of tert-butyl 3-(7-bromo-2-chloro-8-fluoro-6-iodoquinazolin-4-yl)-3,8-diazabicyclo[3.2.1]octane-8-carboxylate (0.5 g, 0.84 mmol) in DMF (100 mL) was added Zn(CN)₂ (0.1 g, 0.88 mmol) and Pd(PPh₃)₄ (0.096 g, 0.08 mmol). The mixture was stirred at 100° C. for 3 hours. The cooled resulting mixture was filtered, the filtrate was concentrated and purified by chromatography column on silica to give the title product (287 mg, 69.1%). MS (ESI, m/e) [M+H]⁺ 496.0, 498.0.

### Step 2: tert-butyl 3-(7-bromo-6-cyano-2,8-difluoroquinazolin-4-yl)-3,8-diazabicyclo[3.2.1]octane-8-carboxylate

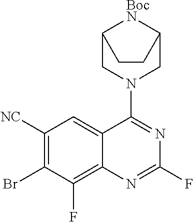 [SMILES: CC(C)(C)OC(=O)N1C2CCC1CN(c1nc(F)nc3c(F)c(Br)c(C#N)cc13)C2]

To a solution of tert-butyl 3-(7-bromo-2-chloro-6-cyano-8-fluoroquinazolin-4-yl)-3,8-diazabicyclo[3.2.1]octane-8-carboxylate (87 mg, 0.176 mmol) in DMSO (6 mL) was added KF (100 mg, 1.76 mmol). Then, the mixture was stirred at 100° C. for 2 hours. Next, the mixture was cooled to room temperature, diluted with EA (20 mL), washed with brine (10 mL), repeated operation 3 times and filtered. The filtrate was concentrated and purified by chromatography column on silica to give the title product (67 mg, 79.7%). MS (ESI, m/e) [M+H]⁺ 480.3, 482.2.

### Step 3: tert-butyl 3-(7-(6-(bis(4-methoxybenzyl)amino)-4-methylpyridin-2-yl)-6-cyano-2,8-difluoroquinazolin-4-yl)-3,8-diazabicyclo[3.2.1]octane-8-carboxylate

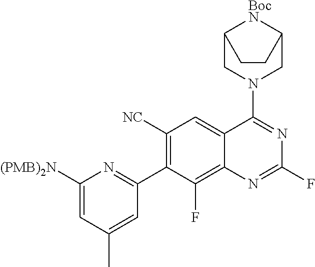

A mixture of tert-butyl 3-(7-(6-(bis(4-methoxybenzyl)amino)-4-methylpyridin-2-yl)-6-cyano-2,8-difluoroquinazolin-4-yl)-3,8-diazabicyclo[3.2.1]octane-8-carboxylate (67 mg, 0.14 mmol), N,N-bis(4-methoxybenzyl)-4-methyl-6-(tributylstannyl)pyridin-2-amine (140 mg, 0.21 mmol), Pd(PPh₃)₄ (17 mg, 0.014 mmol), CuI (11 mg, 0.06 mmol) and LiCl (18 mg, 0.42 mmol) in 1,4-dioxane (10 mL) was stirred at 110° C. for 4 hours. The resulting mixture was filtered, the filtrate was concentrated and purified by column chromatography to give the title product (81 mg, 77.1%). MS (ESI, m/e) [M+1]⁺748.3.

### Step 4: tert-butyl 3-(7-(6-(bis(4-methoxybenzyl)amino)-3-iodo-4-methylpyridin-2-yl)-6-cyano-2,8-difluoroquinolin-4-yl)-3,8-diazabicyclo[3.2.1]octane-8-carboxylate

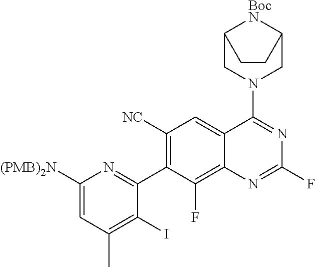

To a mixture of tert-butyl 3-(7-(6-(bis(4-methoxybenzyl)amino)-4-methylpyridin-2-yl)-6-cyano-2,8-difluoroquinazolin-4-yl)-3,8-diazabicyclo[3.2.1]octane-8-carboxylate (200 mg, 0.27 mmol) and Ag₂SO₄ (167 mg, 0.53 mmol) in DMF (5 mL) was added I2 (68 mg, 0.27 mmol). The solution was stirred at room temperature for 8 hours. The resulting mixture was diluted with EA (20 mL), washed with brine (10 mL), repeated operation 3 times, dried overNa₂SO₄ and filtered. The filtrate was concentrated and purified by column chromatography to give the title product (125 mg, 77.1%). MS (ESI, m/e) [M+1]⁺874.2.

### Step 5: tert-butyl 3-(7-(6-(bis(4-methoxybenzyl)amino)-4-methyl-3-(trifluoro methyl)pyridin-2-yl)-6-cyano-2,8-difluoroquinazolin-4-yl)-3,8-diazabicyclo[3.2.1]octane-8-carboxylate

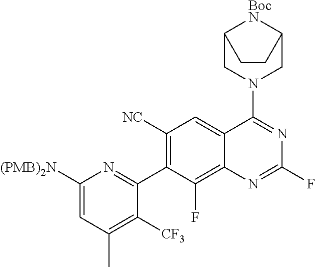

To a mixture of tert-butyl 3-(7-(6-(bis(4-methoxybenzyl)amino)-3-iodo-4-methylpyridin-2-yl)-6-cyano-2,8-difluoroquinolin-4-yl)-3,8-diazabicyclo[3.2.1]octane-8-carboxylate (125 mg, 0.14 mmol) and CuI (272 mg, 1.43 mmol) in NMP (10 mL) was added methyl 2,2-difluoro-2-(fluorosulfonyl)acetate (549 mg, 2.86 mmol). The mixture was stirred at 90° C. for 8 hours. The resulting mixture was cooled to room temperature, then diluted with EA (100 mL), washed with brine (20 mL), repeated operation 3 times, dried over Na₂SO₄ and filtered. The filtrate was concentrated and purified by column chromatography to give the title product (100 mg, 86.2%). MS (ESI, m/e) [M+1]⁺816.3.

### Step 6: tert-butyl 3-(7-(6-(bis(4-methoxybenzyl)amino)-4-methyl-3-(trifluoro methyl)pyridin-2-yl)-6-cyano-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

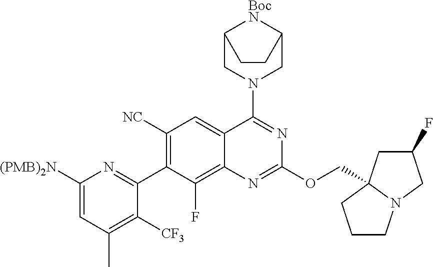

To a solution of ((2R,7aS)-2-fluorotetrahydro-1H-pyrrolizin-7a(5H)-yl)methanol (97 mg, 0.61 mmol) in THF was cooled to 0° C. and NaH (19 mg, 0.49 mmol) was added one portion. The mixture was stirred at 0° C. for 30 min. Then, the solution was added dropwise into the solution of tert-butyl 3-(7-(6-(bis(4-methoxybenzyl)amino)-4-methyl-3-(trifluoromethyl) pyridin-2-yl)-6-cyano-2,8-difluoroquinazolin-4-yl)-3,8-diazabicyclo[3.2.1]octane-8-carboxylate (100 mg, 0.12 mmol) in THF at 0° C. The resulting mixture was stirred at 0° C. to room temperature for 1.5 hours, then cooled to 0° C., diluted with EA (100 mL), washed with brine (20 mL), repeated operation 3 times, dried over Na₂SO₄ and filtered. The filtrate was concentrated and purified by column chromatography to give the title product (60 mg, 51.2%). MS (ESI, m/e) [M+H]⁺ 955.6.

### Step 7: 7-(6-amino-4-methyl-3-(trifluoromethyl)pyridin-2-yl)-4-(3,8-diazabicyclo [3.2.1]octan-3-yl)-8-fluoro-2-(((2R,7aS)-2-fluorotetrahydro-1H-pyrrolizin-7a(5H)-yl)methoxy) quinazoline-6-carbonitrile

To a solution of tert-butyl 3-(7-(6-(bis(4-methoxybenzyl)amino)-4-methyl-3-(trifluoromethyl)pyridin-2-yl)-6-cyano-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (60 mg, 0.06 mmol) in TFA was stirred at room temperature for 3 days, then concentrated and purified by pre-HPLC to give the title product (15 mg, 39.4%). ¹H NMR (500 MHz, CD₃OD) δ 8.26 (s, 1H), 6.63 (s, 1H), 5.40 (d, J=42.4 Hz, 1H), 4.60 (dd, J=10.0 Hz, J=19.2 Hz, 2H), 4.46-4.43 (m, 1H), 4.40-4.37 (d, J=12.0 Hz, 1H), 3.88 (s, 2H), 3.80-3.74 (m, 2H), 3.59-3.43 (m, 3H), 3.22-3.17 (m, 1H), 2.50-2.32 (m, 5H), 2.26-2.23 (m, 1H), 2.15-2.09 (m, 2H), 2.03-1.88 (m, 5H). MS (ESI, m/e) [M+H]⁺ 614.9.

### Example 120: 4-(3,8-diazabicyclo[3.2.1]octan-3-yl)-6-chloro-7-(5-chloro-6-methyl-1H-indazol-4-yl)-8-fluoro-2-(((2R,7aS)-2-fluorotetrahydro-1H-pyrrolizin-7a(5H)-yl)methoxy)quinazoline

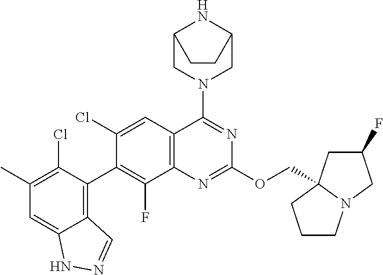 [SMILES: Cc1cc2[nH]ncc2c(-c2c(Cl)cc3c(N4CC5CCC(C4)N5)nc(OC[C@@]45CCCN4C[C@H](F)C5)nc3c2F)c1Cl]

Synthetic Route:

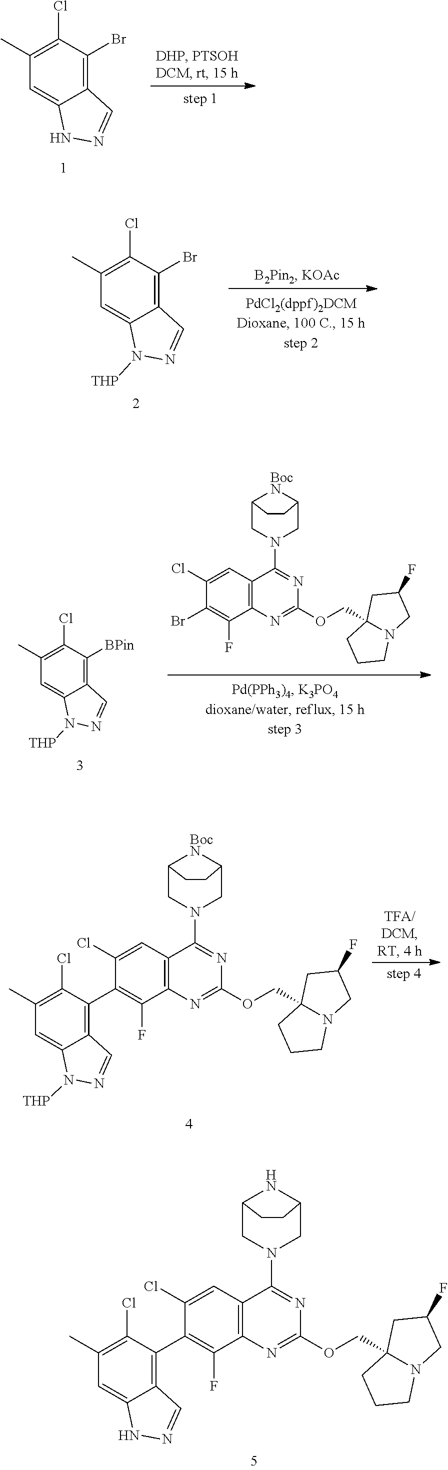

### Step 1: 4-bromo-5-chloro-6-methyl-1-(tetrahydro-2H-pyran-2-yl)-1H-indazole

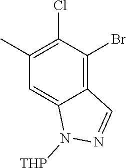

The solution of 4-bromo-5-chloro-6-methyl-1H-indazole (980 mg, 4.0 mmol), PPTS (69 mg, 0.4 mmol) and DHP (403 mg, 4.8 mmol) in DCM (10 mL) was stirred at room temperature for 15 hrs. After completion, the reaction mixture was concentrated, the residue was purified by silica gel (PE:EA=3:1) to give the title product (1200 mg, 90.9%). MS (ESI, m/e) [M+H]⁺ 329, 331.

### Step 2: 5-chloro-6-methyl-1-(tetrahydro-2H-pyran-2-yl)-4-(4,4,5,5-tetramethyl-1,3,2-dioxaborolan-2-yl)-1H-indazole

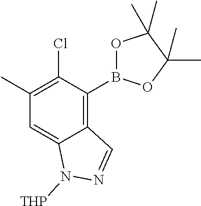

The mixture of 4-bromo-5-chloro-6-methyl-1-(tetrahydro-2H-pyran-2-yl)-1H-indazole (1200 mg, 3.65 mmol), B₂Pin₂ (4620 mg, 1.0 mmol), PdCl₂(dppf)-DCM (300 mg, 0.365 mmol) and KOAc (1070 mg, 11.0 mmol) in dioxane (15 mL) was stirred at 100° C. for 15 hrs. After completion, the reaction mixture was purified by silica gel (PE:EA=1:1) to give the title product (1100 mg, 80.1%), MS (ESI, m/e) [M+H]⁺ 377.

### Step 3: tert-butyl 3-(6-chloro-7-(5-chloro-6-methyl-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy) quinazolin-4-yl)-3,8-diazabicyclo[3.2.1]octane-8-carboxylate

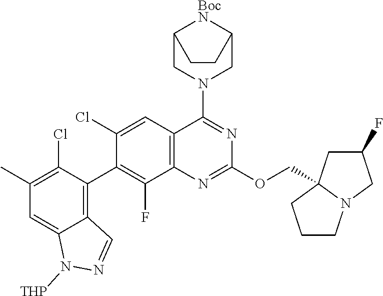

The mixture of tert-butyl 3-(7-bromo-6-chloro-8-fluoro-2-(((2R,7aS)-2-fluoro tetrahydro-1H-pyrrolizin-7a(5H)-yl)methoxy)quinazolin-4-yl)-3,8-diazabicyclo[3.2.1] octane-8-carboxylate (126 mg, 0.2 mmol), 5-chloro-6-methyl-1-(tetrahydro-2H-pyran-2-yl)-4-(4,4,5,5-tetramethyl-1,3,2-dioxaborolan-2-yl)-1H-indazole (266 mg, 0.6 mmol), Pd(PPh₃)₄ (23 mg, 0.02 mmol) and K₃PO₄ (127 mg, 0.6 mmol) in dioxane/H₂O (5 mL/1 mL) was stirred at 100° C. for 15 hrs. After completion, the reaction mixture was purified by Prep-TLC (PE:EA=1:3) to give the title product (226 mg, crude). MS (ESI, m/e) [M+1]⁺798.5.

### Step 4: 4-(3,8-diazabicyclo[3.2.1]octan-3-yl)-6-chloro-7-(5-chloro-6-methyl-1H-indazol-4-yl)-8-fluoro-2-(((2R,7aS)-2-fluorotetrahydro-1H-pyrrolizin-7a(5H)-yl)methoxy)quinazoline

To a solution of tert-butyl 3-(6-chloro-7-(5-chloro-6-methyl-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (226 mg, 0.283 mmol) in DCM (6 mL) was added TFA (3 mL) at room temperature. The resulting mixture was stirred at room temperature for 3 hrs. After completion, the reaction mixture was concentrated and the residue was purified by prep-HPLC to give the title product (26.7 mg, FA salt, 14.4%). ¹H NMR (500 MHz, DMSO-d₆) δ 13.37 (s, 1H), 8.13 (s, 1H), 8.00 (s, 1H), 7.71 (s, 1H), 7.60 (s, 1H), 5.36-5.25 (m, 1H), 4.48-4.46 (m, 2H), 4.49-4.10 (m, 4H), 3.74-3.71 (m, 2H), 3.20-2.81 (m, 4H), 2.54 (s, 3H), 2.25-1.61 (m, 10H). MS (ESI, m/e) [M+H]⁺ 613.9.

### Example 121: 2-(2-(((7-(2-amino-7-fluorobenzo[d]thiazol-4-yl)-4-(3,8-diaza bicyclo[3.2.1]octan-3-yl)-8-fluoro-6-(trifluoromethyl)quinazolin-2-yl)oxy)methyl)-1-methyl pyrrolidin-2-yl)acetonitrile

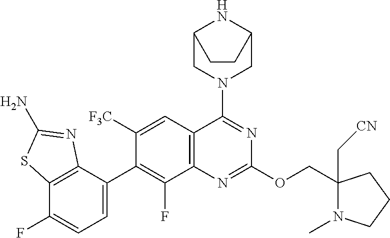 [SMILES: CN1CCCC1(CC#N)COc1nc(N2CC3CCC(C2)N3)c2cc(C(F)(F)F)c(-c3ccc(F)c4sc(N)nc34)c(F)c2n1]

Synthetic Route

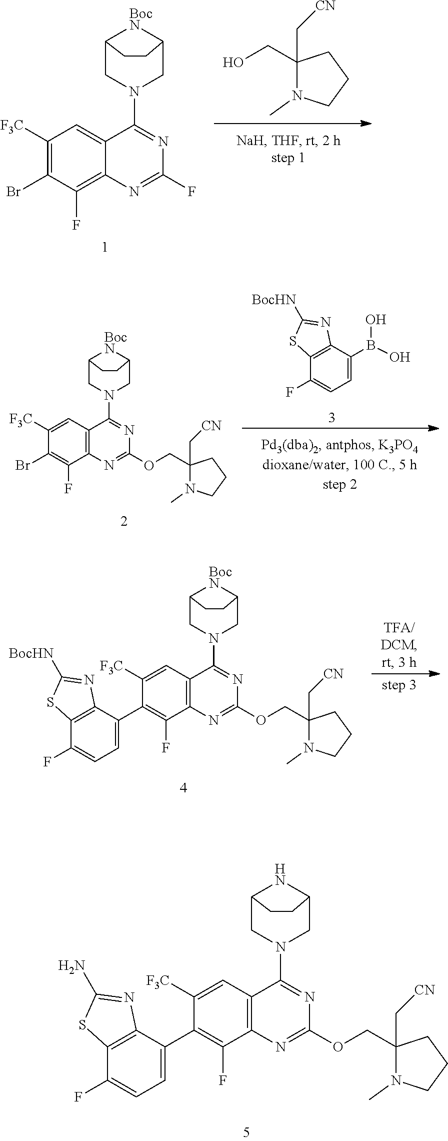 [SMILES: CC(C)(C)OC(=O)N1C2CCC1CN(c1nc(F)nc3c(F)c(Br)c(C(F)(F)F)cc13)C2 ; CN1CCCC1(CO)CC#N ; CN1CCCC1(CC#N)COc1nc(N2CC3CCC(C2)N3C(=O)OC(C)(C)C)c2cc(C(F)(F)F)c(Br)c(F)c2n1 ; CC(C)(C)OC(=O)Nc1nc2c(B(O)O)ccc(F)c2s1 ; CN1CCCC1(CC#N)COc1nc(N2CC3CCC(C2)N3C(=O)OC(C)(C)C)c2cc(C(F)(F)F)c(-c3ccc(F)c4sc(NC(=O)OC(C)(C)C)nc34)c(F)c2n1 ; CN1CCCC1(CC#N)COc1nc(N2CC3CCC(C2)N3)c2cc(C(F)(F)F)c(-c3ccc(F)c4sc(N)nc34)c(F)c2n1]

### Step 1: tert-butyl 3-(7-bromo-2-((2-(cyanomethyl)-1-methylpyrrolidin-2-yl) methoxy)-8-fluoro-6-(trifluoromethyl)quinazolin-4-yl)-3,8-diazabicyclo[3.2.1]octane-8-carboxylate

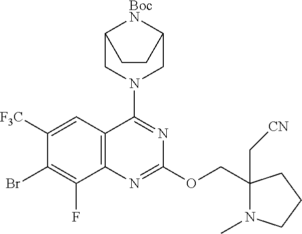 [SMILES: CN1CCCC1(CC#N)COc1nc(N2CC3CCC(C2)N3C(=O)OC(C)(C)C)c2cc(C(F)(F)F)c(Br)c(F)c2n1]

To a solution of 2-(2-(hydroxymethyl)-1-methylpyrrolidin-2-yl)acetonitrile (31 mg, 0.2 mmol) in THF (5 mL) was added sodium hydride (10 mg, 0.4 mmol) at room temperature. The resulting mixture was stirred at room temperature for 0.3 hrs, Then, tert-butyl 3-(7-bromo-2,8-difluoro-6-(trifluoromethyl)quinazolin-4-yl)-3,8-diazabicyclo[3.2.1]octane-8-carboxylate (132 mg, 0.2 mmol) was added to reaction mixture and stirred for 2 hrs at room temperature. After completion, the reaction mixture was purified by Prep-TLC (PE:EA=1:2) to give the title product (80 mg, 61.1%). MS (ESI, m/e) [M+H]⁺ 657.5, 659,5.

### Step 2: tert-butyl 3-(7-(2-((tert-butoxycarbonyl)amino)-7-fluorobenzo[d]thiazol-4-yl)-2-((2-(cyanomethyl)-1-methylpyrrolidin-2-yl)methoxy)-8-fluoro-6-(trifluoromethyl) quinazolin-4-yl)-3,8-diazabicyclo[3.2.1]octane-8-carboxylate

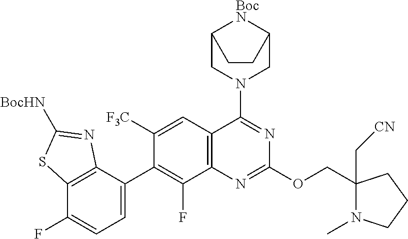 [SMILES: CN1CCCC1(CC#N)COc1nc(N2CC3CCC(C2)N3C(=O)OC(C)(C)C)c2cc(C(F)(F)F)c(-c3ccc(F)c4sc(NC(=O)OC(C)(C)C)nc34)c(F)c2n1]

The mixture of tert-butyl 3-(7-bromo-2-((2-(cyanomethyl)-1-methylpyrrolidin-2-yl)methoxy)-8-fluoro-6-(trifluoromethyl)quinazolin-4-yl)-3,8-diazabicyclo[3.2.1]octane-8-carboxylate (66 mg, 0.1 mmol), (2-((tert-butoxycarbonyl)amino)-7-fluorobenzo[d]thiazol-4-yl)boronic acid (94 mg, 0.3 mmol), Antphos (7.4 mg, 0.02 mmol), Pd₂(dba)₃ (9 mg, 0.01 mmol), and K₃PO₄ (64 mg, 0.3 mmol) in dioxane/H₂O (5 mL/1 mL) was stirred at 100° C. for 5 hrs under N₂ atmosphere. After completion, the reaction mixture was purified by Prep-TLC (PE:EA=1:2) to give the title product (25 mg, 29.6%). MS (ESI, m/e) [M+H]⁺ 844.5.

### Step 3: 2-(2-(((7-(2-amino-7-fluorobenzo[d]thiazol-4-yl)-4-(3,8-diazabicyclo [3.2.1]octan-3-yl)-8-fluoro-6-(trifluoromethyl)quinazolin-2-yl)oxy)methyl)-1-methylpyrrolidin-2-yl)acetonitrile

To a solution of tert-butyl 3-(7-(2-((tert-butoxycarbonyl)amino)-7-fluorobenzo [d]thiazol-4-yl)-2-((2-(cyanomethyl)-1-methylpyrrolidin-2-yl)methoxy)-8-fluoro-6-(trifluoro methyl)quinazolin-4-yl)-3,8-diazabicyclo[3.2.1]octane-8-carboxylate (25 mg, 0.0296 mmol) in DCM (4 mL) was added TFA (2 mL) at room temperature. The resulting mixture was stirred at room temperature for 3 hrs. After completion, the reaction mixture was concentrated and the residue was purified by prep-HPLC to give the title product (12.0 mg, FA salt, 60.0%). ¹H NMR (500 MHz, CD₃OD) δ 8.46 (s, 1H), 8.13 (s, 1H), 7.25-7.13 (m, 1H), 6.99-6.95 (m, 1H), 4.68-4.64 (m, 2H), 4.52-4.43 (m, 2H), 4.05 (s, 2H), 3.85-3.80 (m, 2H), 3.01-2.86 (m, 3H), 2.80-2.76 (m, 1H), 2.52 (s, 3H), 2.14-1.79 (m, 8H). MS (ESI, m/e) [M+H]⁺ 645.0.

### Example 122: 4-(4-(3-oxa-7,9-diazabicyclo[3.3.1]nonan-9-yl)-8-fluoro-2-(((2R,4R)-4-fluoro-1,2-dimethylpyrrolidin-2-yl)methoxy)-6-(trifluoromethyl)quinazolin-7-yl)-7-fluorobenzo[d]thiazol-2-amine

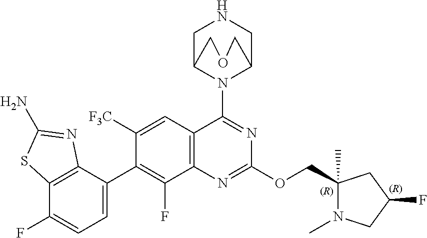

Synthetic Route

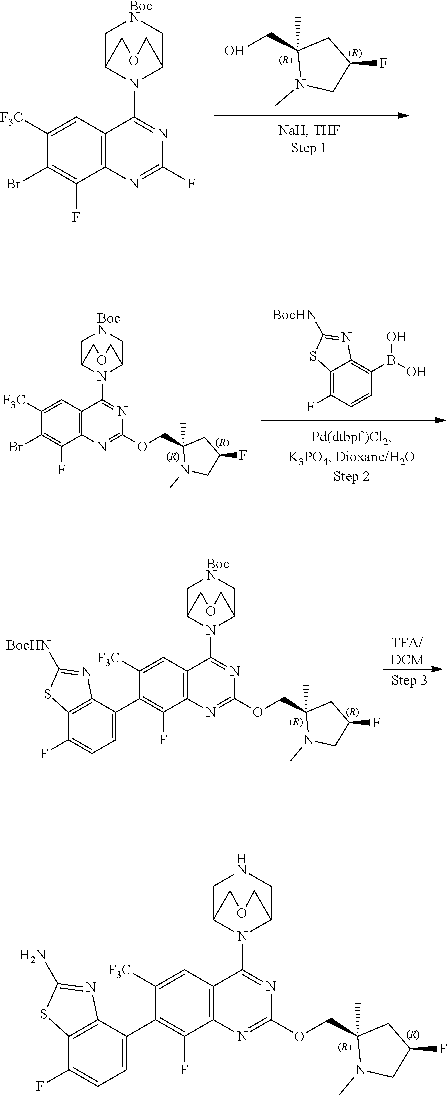

### Step 1: tert-butyl 9-(7-bromo-8-fluoro-2-(((2R,4R)-4-fluoro-1,2-dimethyl pyrrolidin-20 yl)methoxy)-6-(trifluoromethyl)quinazolin-4-yl)-3-oxa-7,9-diazabicyclo[3.3.1] nonane-7-carboxylate

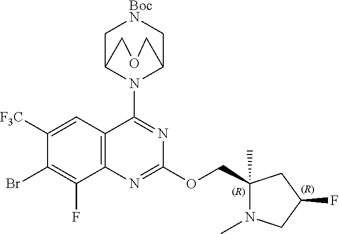

To a solution of ((2R,4R)-4-fluoro-1,2-dimethylpyrrolidin-2-yl)methanol (48.54 mg, 0.33 mmol) in THF (5 mL) was added sodium hydride (13.42 mg, 0.33 mmol) at room temperature. The resulting mixture was stirred at room temperature for 1 hrs. Then tert-butyl 9-(7-bromo-2,8-difluoro-6-(trifluoromethyl)quinazolin-4-yl)-3-oxa-7,9-diazabicyclo[3.3.1]nonane-7-carboxylate (150.7 mg, 0.28 mmol) was added to the reaction mixture and stirred at room temperature for 1 hrs. After completion, the reaction mixture was diluted with EA (50 mL), washed with saturated NaCl (15 mL×3). The organic layer was dried over anhydrous Na₂SO₄, filtered and the filtrate was concentrated to give the residue. The residue was purified by Prep-TLC (PE:EA=3:1) to give the title product (150 mg, 0.23 mmol). MS (ESI, m/e) [M+H]⁺ 666.2, 668.2.

### Step 2: tert-butyl 9-(7-(2-((tert-butoxycarbonyl)amino)-7-fluorobenzo[d]thiazol-4-yl)-8-fluoro-2-(((2R,4R)-4-fluoro-1,2-dimethylpyrrolidin-2-yl)methoxy)-6-(trifluoromethyl) quinazolin-4-yl)-3-oxa-7,9-diazabicyclo [3.3.1]nonane-7-carboxylate

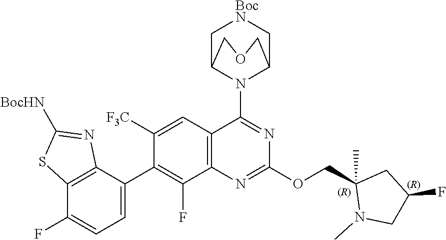

To a solution of tert-butyl 9-(7-bromo-8-fluoro-2-(((2R,4R)-4-fluoro-1,2-dimethylpyrrolidin-2-yl)methoxy)-6-(trifluoromethyl)quinazolin-4-yl)-3-oxa-7,9-diaza bicyclo[3.3.1]nonane-7-carboxylate (150 mg, 0.23 mmol), (2-amino-7-fluorobenzo[d]thiazol-4-yl)boronic acid (93.6 mg, 0.3 mmol) and Pd(dtbpf)Cl₂ (12.9 mg, 0.02 mmol) in dioxane/H₂O (5 mL/1 mL) was added K₃PO₄ (127.2 mg, 0.60 mmol) at room temperature. The resulting mixture was stirred at 100° C. for 16 hrs. After completion, the reaction mixture was diluted with EA (30 mL), washed with saturated NaCl (250 mL×3). The organic layer was dried over anhydrous Na₂SO₄, filtered and the filtrate was concentrated to give the residue. The residue was purified by Prep-TLC (pure EA) to give the title product (100 mg, 0.12 mmol). MS (ESI, m/e) [M+1]⁺854.3.

### Step 3: 4-(4-(3-oxa-7,9-diazabicyclo[3.3.1]nonan-9-yl)-8-fluoro-2-(((2R,4R)-4-fluoro-1,2-dimethylpyrrolidin-2-yl)methoxy)-6-(trifluoromethyl)quinazolin-7-yl)-7-fluorobenzo[d]thiazol-2-amine

To a solution of tert-butyl 9-(7-(2-((tert-butoxycarbonyl)amino)-7-fluorobenzo [d]thiazol-4-yl)-8-fluoro-2-(((2R,4R)-4-fluoro-1,2-dimethylpyrrolidin-2-yl)methoxy)-6-(trifluoromethyl)quinazolin-4-yl)-3-oxa-7,9-diazabicyclo[3.3.1]nonane-7-carboxylate (100 mg, 0.12 mmol) in DCM (5 mL) was added TFA (5 mL) at room temperature. The resulting mixture was stirred at room temperature for 3 hrs. After completion, the reaction mixture was concentrated to give the residue. The residue was purified by Pre-HPLC to give the title product (31.12 mg). 1H NMR (500 MHz, DMSO-d₆) δ 7.93-7.88 (m, 3H), 7.20-7.17 (m, 1H), 7.06-7.02 (m, 1H), 5.26-5.14 (m, 1H), 4.36-4.05 (m, 10H), 3.28-3.23 (m, 4H), 2.90-2.85 (m, 1H), 2.31-2.27 (s, 4H), 1.87-1.81 (m, 1H), 1.14 (s, 3H). MS (ESI, m/e) [M+H]⁺ 654.1.

### Example 123: 3-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-5-chloro-4-cyclopropylphenol

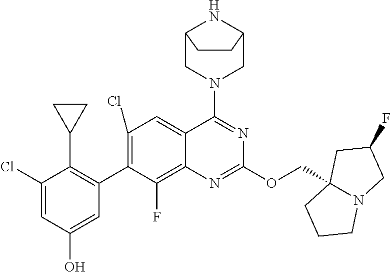 [SMILES: Oc1cc(Cl)c(C2CC2)c(-c2c(Cl)cc3c(N4CC5CCC(C4)N5)nc(OC[C@@]45CCCN4C[C@H](F)C5)nc3c2F)c1]

Synthetic Route

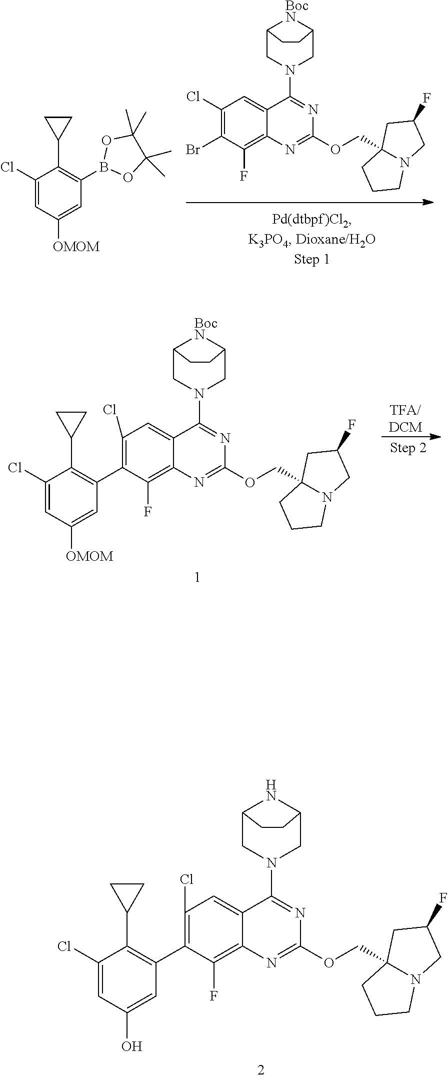

### Step 1: tert-butyl 3-(6-chloro-7-(3-chloro-2-cyclopropyl-5-(methoxymethoxy) phenyl)-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

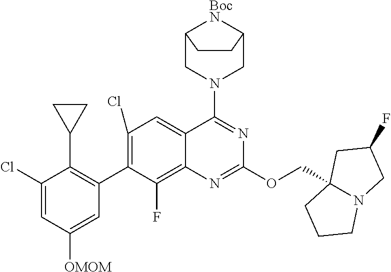

To a solution of tert-butyl 3-(7-bromo-6-chloro-8-fluoro-2-(((2R,7aS)-2-fluoro tetrahydro-1H-pyrrolizin-7a(5H)-yl)methoxy)quinazolin-4-yl)-3,8-diazabicyclo[3.2.1] octane-8-carboxylate (138.0 mg, 0.22 mmol), (2-amino-7-fluorobenzo[d]thiazol-4-yl)boronic acid (93.6 mg, 0.3 mmol) and Pd(dtbpf)Cl₂ (12.9 mg, 0.02 mmol) in dioxane/H₂O (5 mL/1 mL) was added K₃PO₄ (127.2 mg, 0.60 mmol) at room temperature. The resulting mixture was stirred at 100° C. for 16 hrs. After completion, the reaction mixture was diluted with EA (30 mL), washed with brine (250 mL×3). The organic layer was dried over anhydrous Na₂SO₄, filtered and the filtrate was concentrated to give the residue. The residue was purified by Prep-TLC (pure EA) to give the title product (80.0 mg, 0.11 mmol). MS (ESI, m/e) [M+H]⁺ 760.3.

### Step 2: 3-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-5-chloro-4-cyclopropyl phenol

To a solution of tert-butyl 3-(6-chloro-7-(3-chloro-2-cyclopropyl-5-(methoxy methoxy)phenyl)-8-fluoro-2-(((2R,7aS)-2-fluorotetrahydro-1H-pyrrolizin-7a(5H)-yl)methoxy) quinazolin-4-yl)-3,8-diazabicyclo[3.2.1]octane-8-carboxylate (80.0 mg, 0.11 mmol) in DCM (4 mL) was added TFA (4 mL) at room temperature. The resulting mixture was stirred at room temperature for 3 hrs. After completion, the reaction mixture was concentrated to give the residue. The residue was purified by Pre-HPLC to give the title product (8.38 mg). ¹H NMR (500 MHz, DMSO-d₆) δ 8.19 (s, 1H), 7.88 (s, 1H), 6.94 (s, 1H), 6.56 (s, 1H), 5.33-5.22 (m, 1H), 4.36-4.23 (m, 2H), 4.10-3.98 (m, 2H), 3.66-3.59 (m, 5H), 3.09-3.02 (m, 3H), 2.83-2.82 (m, 1H), 2.11-2.00 (m, 3H), 1.85-1.62 (m, 8H), 0.58-0.52 (m, 2H), 0.15-014 (m, 2H). MS (ESI, m/e) [M+H]⁺ 616.4.

### Example 124: 2-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-6-amino-3-iodo-4-methylbenzonitrile

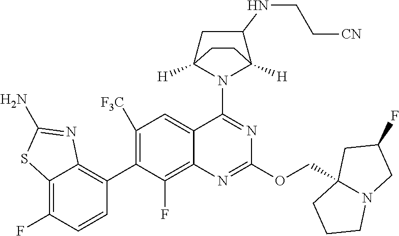 [SMILES: N#CCCNC1C[C@H]2CC[C@@H]1N2c1nc(OC[C@@]23CCCN2C[C@H](F)C3)nc2c(F)c(-c3ccc(F)c4sc(N)nc34)c(C(F)(F)F)cc12]

Synthetic Route:

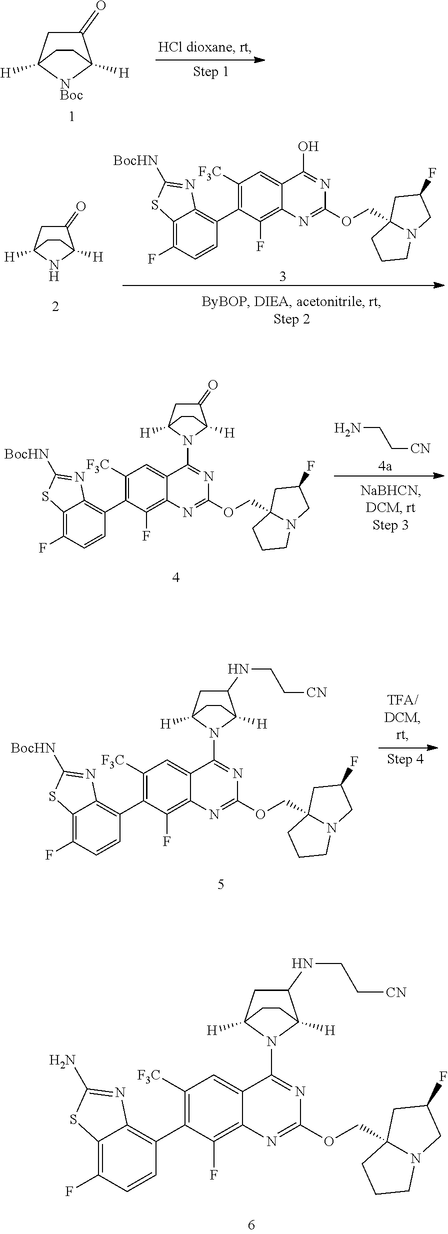

### Step 1: (1S,4R)-7-azabicyclo[2.2.1]heptan-2-one

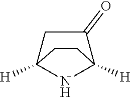

The solution of tert-butyl (1S,4R)-2-oxo-7-azabicyclo[2.2.1]heptane-7-carboxylate (426 mg, 2.0 mmol) in HCl gas dioxane (4N, 2 mL) and DCM (2 mL) was stirred at room temperature for 2 hrs. After completion, the reaction mixture was concentrated and the title product was directly used next step without further purification. MS (ESI, m/e) [M+H]⁺ 112.4.

### Step 2: tert-butyl (7-fluoro-4-(8-fluoro-2-(((2R,7aS)-2-fluorotetrahydro-1H-pyrrolizin-7a(5H)-yl)methoxy)-4-((1S,4R)-2-oxo-7-azabicyclo[2.2.1]heptan-7-yl)-6-(trifluoro methyl)quinazolin-7-yl)benzo[d]thiazol-2-yl)carbamate

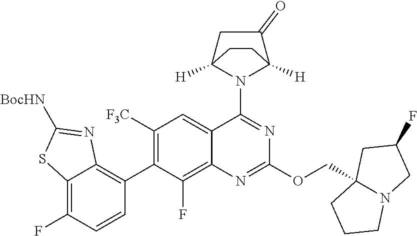 [SMILES: CC(C)(C)OC(=O)Nc1nc2c(-c3c(C(F)(F)F)cc4c(N5[C@@H]6CC[C@H]5C(=O)C6)nc(OC[C@@]56CCCN5C[C@H](F)C6)nc4c3F)ccc(F)c2s1]

The mixture of tert-butyl (7-fluoro-4-(8-fluoro-2-(((2R,7aS)-2-fluorotetra hydro-1H-pyrrolizin-7a(5H)-yl)methoxy)-4-hydroxy-6-(trifluoromethyl)quinazolin-7-yl)benzo[d] thiazol-2-yl)carbamate (66 mg, 0.1 mmol), (1S,4R)-7-azabicyclo[2.2.1] heptan-2-one (30 mg, 0.2 mmol), ByBOP (78 mg, 0.15 mmol) and DIEA (52 mg, 0.4 mmol) in CH₃CN (5 mL) was stirred at 25° C. for 15 hrs under N₂ atmosphere. After completion, the reaction mixture was purified by Prep-TLC (PE:EA=1:1) to give the title product (40 mg, 53.5%). MS (ESI, m/e) [M+H]⁺ 749.5.

### Step 3: tert-butyl (4-(4-((1S,4R)-2-((2-cyanoethyl)amino)-7-azabicyclo [2.2.1] heptan-7-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-yl) carbamate

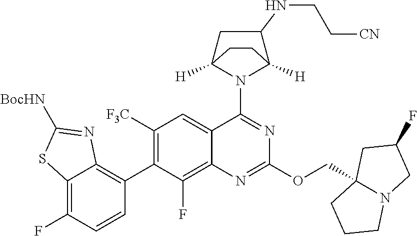 [SMILES: CC(C)(C)OC(=O)Nc1nc2c(-c3c(C(F)(F)F)cc4c(N5[C@@H]6CC[C@H]5C(NCCC#N)C6)nc(OC[C@@]56CCCN5C[C@H](F)C6)nc4c3F)ccc(F)c2s1]

The mixture of tert-butyl (7-fluoro-4-(8-fluoro-2-(((2R,7aS)-2-fluorotetrahydro-1H-pyrrolizin-7a(5H)-yl)methoxy)-4-((1S,4R)-2-oxo-7-azabicyclo[2.2.1]heptan-7-yl)-6-(trifluoromethyl)quinazolin-7-yl)benzo[d]thiazol-2-yl)carbamate (75.0 mg, 0.1 mmol), 3-amino propanenitrile (140 mg, 2.0 mmol) and NaBH₃CN (126 mg, 2.0 mmol) in DCM/H₂O (4 mL/2 mL) was stirred at 25° C. for 12 hrs. After completion, the reaction mixture was purified by Prep-TLC (PE:EA=1:1) to give the title product (60 mg, 75.2%). MS (ESI, m/e) [M+H]⁺ 803.4.

### Step 4: 3-(((1S,4R)-7-(7-(2-amino-7-fluorobenzo[d]thiazol-4-yl)-8-fluoro-2-(((2R,7aS)-2-fluorotetrahydro-1H-pyrrolizin-7a(5H)-yl)methoxy)-6-(trifluoro methyl) quinazolin-4-yl)-7-azabicyclo[2.2.1]heptan-2-yl)amino)propanenitrile

To a solution of tert-butyl (4-(4-((1S,4R)-2-((2-cyanoethyl)amino)-7-azabicyclo [2.2.1]heptan-7-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-yl) carbamate (60 mg, 0.0647 mmol) in DCM (2 mL) was added TFA (2 mL) at room temperature. The resulting mixture was stirred at room temperature for 3 hrs. After completion, the reaction mixture was concentrated and the residue was purified by prep-HPLC to give the title product (14.5 mg, 32.2%). ¹H NMR (500 MHz, CD₃OD) δ 8.21 (s, 1H), 7.22-7.19 (m, 1H), 6.99-6.96 (m, 1H), 5.52-5.42 (m, 1H), 4.97-4.90 (m, 2H), 4.57-4.52 (m, 2H), 3.75-3.61 (m, 3H), 3.51-3.46 (m, 2H), 2.89-2.87 (m, 2H), 2.62-2.51 (m, 5H), 2.49-2.21 (m, 4H), 2.10-1.74 (m, 4H), 1.35-1.16 (m, 1H). MS (ESI, m/e) [M+H]⁺ 703.5.

### Example 125: 6-(4-(3,8-diazabicyclo[3.2.1]octan-3-yl)-6-chloro-8-fluoro-2-(((2R,7aS)-2-fluoro tetrahydro-1H-pyrrolizin-7a(5H)-yl)methoxy)quinazolin-7-yl)-5-(trifluoro methyl)-4-vinylpyridin-2-amine

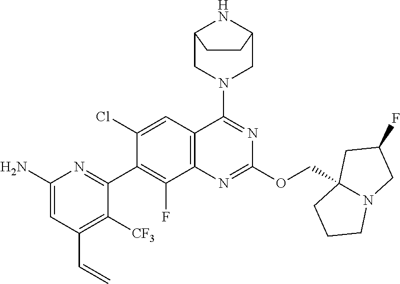 [SMILES: C=Cc1cc(N)nc(-c2c(Cl)cc3c(N4CC5CCC(C4)N5)nc(OC[C@@]45CCCN4C[C@H](F)C5)nc3c2F)c1C(F)(F)F]

Synthetic Route

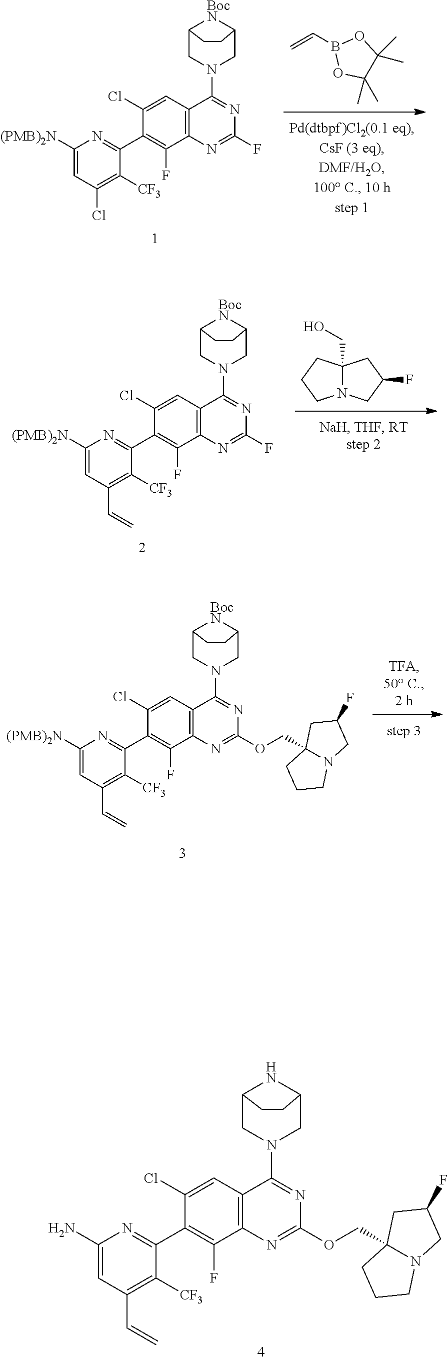

### Step 1: tert-butyl 3-(7-(6-(bis(4-methoxybenzyl)amino)-3-(trifluoromethyl)-4-vinylpyridin-2-yl)-6-chloro-2,8-difluoroquinazolin-4-yl)-3,8-diazabicyclo[3.2.1]octane-8-carboxylate

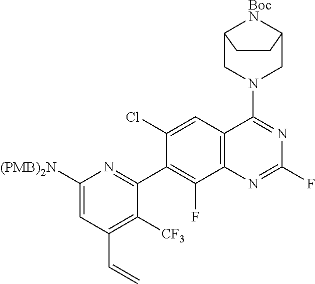

Into a 50-mL round-bottom flask was placed tert-butyl 3-(7-(6-(bis(4-methoxy benzyl)amino)-4-chloro-3-(trifluoromethyl)pyridin-2-yl)-6-chloro-2,8-difluoroquinazolin-4-yl)-3,8-diazabicyclo[3.2.1]octane-8-carboxylate (200.0 mg, 0.24 mmol), DMF (10 mL), H₂O (2.5 mL), CsF (108.0 mg, 0.71 mmol), 4,4,5,5-tetramethyl-2-vinyl-1,3,2-dioxaborolane (55.0 mg, 0.36 mmol) and Pd(dtbpf)Cl₂ (15.3 mg, 0.024 mmol). The resulting solution was stirred for 10 h at 100° C. The resulting mixture was concentrated under vacuum. The crude product was purified by Prep-TLC PE:EA=2:1 within 45; Detector, 254/280 nm. This resulted in 130 mg (65.65%) of the title product. MS (ESI, m/e) [M+H]⁺ 837.

### Step 2: tert-butyl 3-(7-(6-(bis(4-methoxybenzyl)amino)-3-(trifluoromethyl)-4-vinylpyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

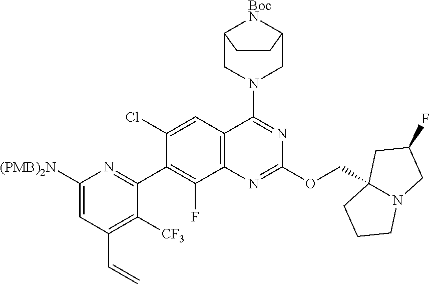

To a solution of (2R,7aS)-2-fluorotetrahydro-1H-pyrrolizin-7a(5H)-yl)methanol (49.4 mg, 0.311 mmol) in THF (extra dry, 4 mL) was added NaH (24.8 mg, 0.62 mmol) at rt and stirred for 30 min. The tert-butyl 3-(7-(6-(bis(4-methoxybenzyl)amino)-3-(trifluoromethyl)-4-vinylpyridin-2-yl)-6-chloro-2,8-difluoroquinazolin-4-yl)-3,8-diazabicyclo[3.2.1]octane-8-carboxylate (130 mg, 0.155 mmol) was added in it at rt and stirred for 2 h. The resulting solution was diluted with 50 mL H₂O, extracted with 80 mL ethyl acetate, washed with 3×50 ml of brine, concentrated under vacuum and purified by Prep-TLC with PE:EA=2:1; Detector, 254/280 nm. This resulted in 100 mg (66.22%) of the title product. MS (ESI, m/e) [M+H]⁺ 976.

### Step 3: 6-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-5-(trifluoromethyl)-4-vinyl pyridin-2-amine

Into a 25-mL round-bottom flask was placed tert-butyl 3-(7-(6-(bis(4-methoxy benzyl)amino)-3-(trifluoromethyl)-4-vinylpyridin-2-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1] octane-8-carboxylate (80 mg, 0.082 mmol) and TFA (2 mL). The resulting solution was stirred at 50° C. for 2 h in an oil bath. Then, the mixture was concentrated under vacuum. The crude product was purified by Prep-HPLC to give the title product (22.4 mg, 52%). ¹H NMR (300 MHz, CD₃OD) δ 7.97 (d, J=1.8 Hz, 1H), 7.11-6.94 (m, 2H), 5.97 (d, J=17.1 Hz, 1H), 5.71-5.59 (m, 1H), 4.71 (d, J=7.8 Hz, 4H), 4.26 (s, 2H), 4.04-3.81 (m, 5H), 3.50 (ddt, J=16.2, 10.6, 5.7 Hz, 1H), 2.89-2.72 (m, 1H), 2.70-2.57 (m, 1H), 2.38 (ddt, J=16.5, 11.2, 7.1 Hz, 4H), 2.14 (s, 5H). MS (ESI, m/e) [M+H]⁺ 636.10.

### Example 126: 3-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-5-fluoro-4-(trifluoromethyl)phenol

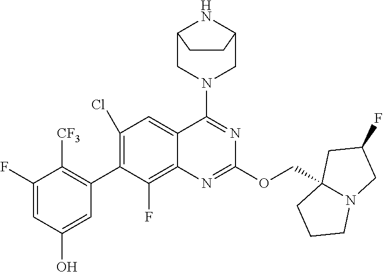 [SMILES: Oc1cc(F)c(C(F)(F)F)c(-c2c(Cl)cc3c(N4CC5CCC(C4)N5)nc(OC[C@@]45CCCN4C[C@H](F)C5)nc3c2F)c1]

Example 126 (25 mg, FA salt) can be delivered via the same procedures as Example 105 by using 1-bromo-3-fluoro-2-(trifluoromethyl)benzene instead of 1-bromo-3-chloro-2-(trifluoromethyl) benzene. ¹H NMR (400 MHz, CD₃OD) δ 7.92 (s, 1H), 6.83 (d, J=13.3 Hz, 1H), 6.52 (s, 1H), 5.47 (d, J=53.1 Hz, 1H), 4.64-4.47 (m, 5H), 4.04 (s, 2H), 3.84-3.58 (m, 5H), 2.61-2.43 (m, 2H), 2.40-2.31 (m, 1H), 2.28-2.15 (m, 2H), 2.14-1.96 (m, 5H). MS (ESI, m/e) [M+H]⁺ 628.4.

### Example 127: 4-(4-(3,6-diazabicyclo[3.1.1]heptan-6-yl)-8-fluoro-2-(((2R,7aS)-2-fluorotetrahydro-1H-pyrrolizin-7a(5H)-yl)methoxy)-6-(trifluoromethyl)quinazolin-7-yl)-7-fluoro benzo[d]thiazol-2-amine

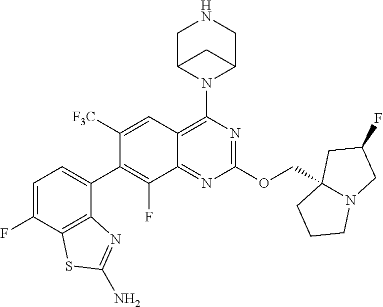 [SMILES: Nc1nc2c(-c3c(C(F)(F)F)cc4c(N5C6CNCC5C6)nc(OC[C@@]56CCCN5C[C@H](F)C6)nc4c3F)ccc(F)c2s1]

Example 127 (3 mg) was delivered via the same procedure as in Example 95 by using tert-butyl 3,6-diazabicyclo[3.1.1]heptane-3-carboxylate instead of (1S,4R)—N-(3,3-difluoropropyl)-7-aza bicyclo [2.2.1] heptan-2-amine. ¹H NMR (400 MHz, CD₃OD) δ 8.04 (s, 1H), 7.08-7.06 (m, 1H), 7.21-7.18 (m, 1H), 7.00-6.96 (m, 1H), 5.53 (d, J=52.1 Hz, 1H), 5.22-5.10 (m, 2H), 4.64 (s, 2H), 4.06-3.70 (m, 5H), 3.61-3.57 (m, 3H), 3.30-3.25 (m, 1H), 2.62-2.50 (m, 2H), 2.44-2.24 (m, 3H), 2.16-2.08 (m, 2H). MS (ESI, m/e) [M+H]⁺ 636.4.

### Example 128: 4-(4-(3,8-diazabicyclo[3.2.1]octan-3-yl)-2-(((2R,7aS)-2-fluorotetra hydro-1H-pyrrolizin-7a(5H)-yl)methoxy)-6-(trifluoromethyl)quinazolin-7-yl)-7-fluorobenzo[d] thiazol-2-amine

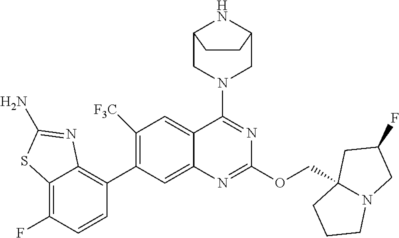 [SMILES: Nc1nc2c(-c3cc4nc(OC[C@@]56CCCN5C[C@H](F)C6)nc(N5CC6CCC(C5)N6)c4cc3C(F)(F)F)ccc(F)c2s1]

Synthetic Route

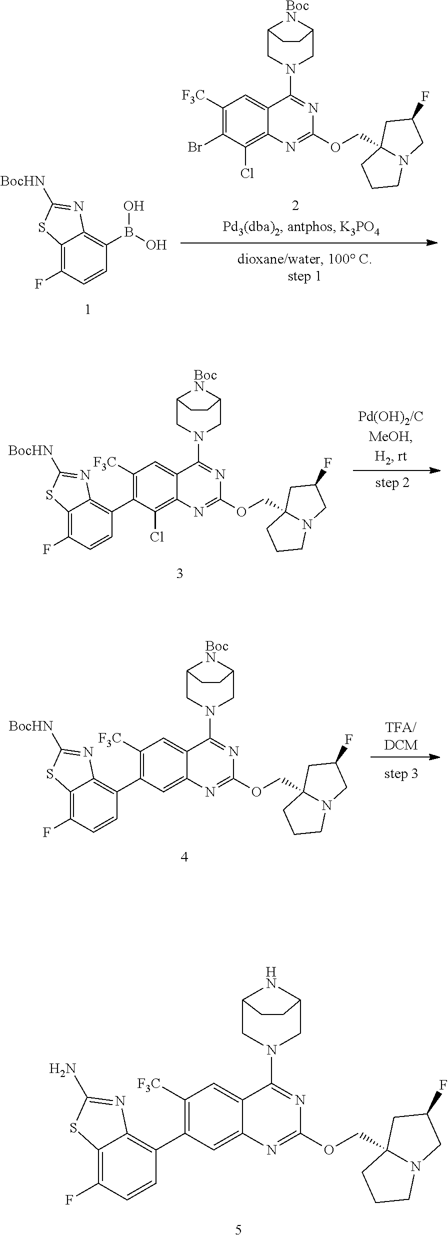 [SMILES: CC(C)(C)OC(=O)Nc1nc2c(B(O)O)ccc(F)c2s1 ; CC(C)(C)OC(=O)N1C2CCC1CN(c1nc(OC[C@@]34CCCN3C[C@H](F)C4)nc3c(Cl)c(Br)c(C(F)(F)F)cc13)C2 ; CC(C)(C)OC(=O)Nc1nc2c(-c3c(C(F)(F)F)cc4c(N5CC6CCC(C5)N6C(=O)OC(C)(C)C)nc(OC[C@@]56CCCN5C[C@H](F)C6)nc4c3Cl)ccc(F)c2s1 ; CC(C)(C)OC(=O)Nc1nc2c(-c3cc4nc(OC[C@@]56CCCN5C[C@H](F)C6)nc(N5CC6CCC(C5)N6C(=O)OC(C)(C)C)c4cc3C(F)(F)F)ccc(F)c2s1 ; Nc1nc2c(-c3cc4nc(OC[C@@]56CCCN5C[C@H](F)C6)nc(N5CC6CCC(C5)N6)c4cc3C(F)(F)F)ccc(F)c2s1]

### Step 1: tert-butyl 3-(7-(2-((tert-butoxycarbonyl)amino)-7-fluorobenzo[d]thiazol-4-yl)-8-chloro-2-(((2R,7aS)-2-fluorotetrahydro-1H-pyrrolizin-7a(5H)-yl)methoxy)-6-(trifluoro methyl)quinazolin-4-yl)-3,8-diazabicyclo[3.2.1]octane-8-carboxylate

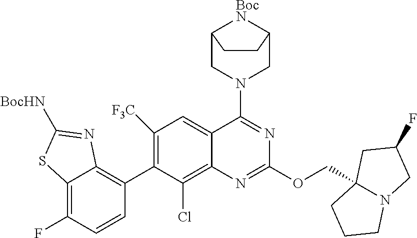 [SMILES: CC(C)(C)OC(=O)Nc1nc2c(-c3c(C(F)(F)F)cc4c(N5CC6CCC(C5)N6C(=O)OC(C)(C)C)nc(OC[C@@]56CCCN5C[C@H](F)C6)nc4c3Cl)ccc(F)c2s1]

The mixture of tert-butyl 3-(7-bromo-8-chloro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1] octane-8-carboxylate (200 mg, 0.295 mmol), (2-((tert-butoxycarbonyl)amino)-7-fluorobenzo[d] thiazol-4-yl)boronic acid (460 mg, 1.5 mmol), Pd₂(dba)₃ (81 mg, 0.0885 mmol), (R)-Antphos (65 mg, 0.177 mmol) and K₃PO₄ (212 mg, 1.0 mmol) in dioxane/H₂O (10 mL/2 mL) was stirred at 100° C. for 4 hrs. After completion, the reaction mixture was purified by Prep-TLC (PE:EA=1:1) to give the title product (170 mg, 56.7%), MS (ESI, m/e) [M+1]⁺866.5.

### Step 2: tert-butyl 3-(7-(2-((tert-butoxycarbonyl)amino)-7-fluorobenzo[d]thiazol-4-yl)-2-(((2R,7aS)-2-fluorotetrahydro-1H-pyrrolizin-7a(5H)-yl)methoxy)-6-(trifluoromethyl) quinazolin-4-yl)-3,8-diazabicyclo[3.2.1]octane-8-carboxylate

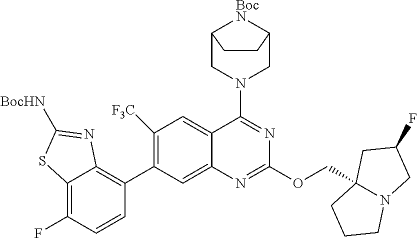 [SMILES: CC(C)(C)OC(=O)Nc1nc2c(-c3cc4nc(OC[C@@]56CCCN5C[C@H](F)C6)nc(N5CC6CCC(C5)N6C(=O)OC(C)(C)C)c4cc3C(F)(F)F)ccc(F)c2s1]

The solution of Tert-butyl 3-(7-(2-((tert-butoxycarbonyl)amino)-7-fluorobenzo[d]thiazol-4-yl)-8-chloro-2-(((2R,7aS)-2-fluorotetrahydro-1H-pyrrolizin-7a(5H)-yl)methoxy)-6-(trifluoromethyl)quinazolin-4-yl)-3,8-diazabicyclo[3.2.1] octane-8-carboxylate (20 mg, 0.023 mmol) and Pd(OH)₂ under H₂ gas in methanol (5 mL) was stirred at room temperature for 4 hrs. After completion, the reaction mixture was filtered and concentrated. The title product was directly used next step without further purification. MS (ESI, m/e) [M+H]⁺ 832.4.

### Step 3: 4-(4-(3,8-diazabicyclo[3.2.1]octan-3-yl)-2-(((2R,7aS)-2-fluorotetrahydro-1H-pyrrolizin-7a(5H)-yl)methoxy)-6-(trifluoromethyl)quinazolin-7-yl)-7-fluorobenzo[d]thiazol-2-amine

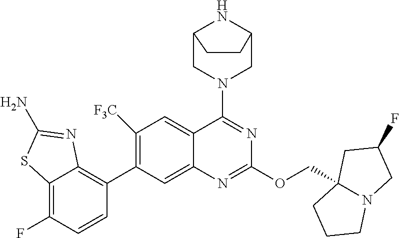 [SMILES: Nc1nc2c(-c3cc4nc(OC[C@@]56CCCN5C[C@H](F)C6)nc(N5CC6CCC(C5)N6)c4cc3C(F)(F)F)ccc(F)c2s1]

To a solution of tert-butyl 3-(7-(2-((tert-butoxycarbonyl)amino)-7-fluorobenzo[d]thiazol-4-yl)-2-(((2R,7aS)-2-fluorotetrahydro-1H-pyrrolizin-7a(5H)-yl)methoxy)-6-(trifluoro methyl)quinazolin-4-yl)-3,8-diazabicyclo[3.2.1]octane-8-carboxylate (6.0 mg, 0.00722 mmol) in DCM (4 mL) was added TFA (2 mL) at room temperature. The resulting mixture was stirred at room temperature for 4 hrs. After completion, the reaction mixture was concentrated to give the residue. The residue was purified by Prep-HPLC to give the title product (0.55 mg, 12.1%). ¹H NMR (500 MHz, CD₃OD) δ 8.37 (s, 1H), 7.57 (s, 1H), 7.24-7.11 (m, 1H), 6.96-6.93 (m, 1H), 5.56-5.46 (m, 1H), 5.06-4.97 (m, 2H), 4.58-4.50 (m, 6H), 3.71 (s, 2H), 3.46-3.40 (m, 2H), 2.58-1.99 (m, 10H). MS (ESI, m/e) [M+1]⁺632.4.

### Example 129: 6-(4-(3,8-diazabicyclo[3.2.1]octan-3-yl)-6-cyclopropyl-8-fluoro-2-(((2R,7aS)-2-fluorotetrahydro-1H-pyrrolizin-7a(5H)-yl)methoxy)quinazolin-7-yl)-4-methyl-5-(trifluoromethyl)pyridin-2-amine

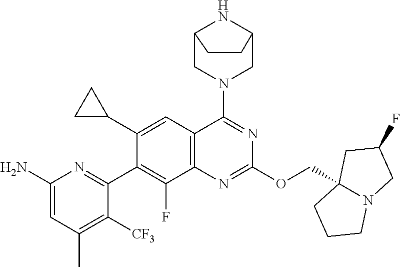 [SMILES: Cc1cc(N)nc(-c2c(C3CC3)cc3c(N4CC5CCC(C4)N5)nc(OC[C@@]45CCCN4C[C@H](F)C5)nc3c2F)c1C(F)(F)F]

Synthetic Route

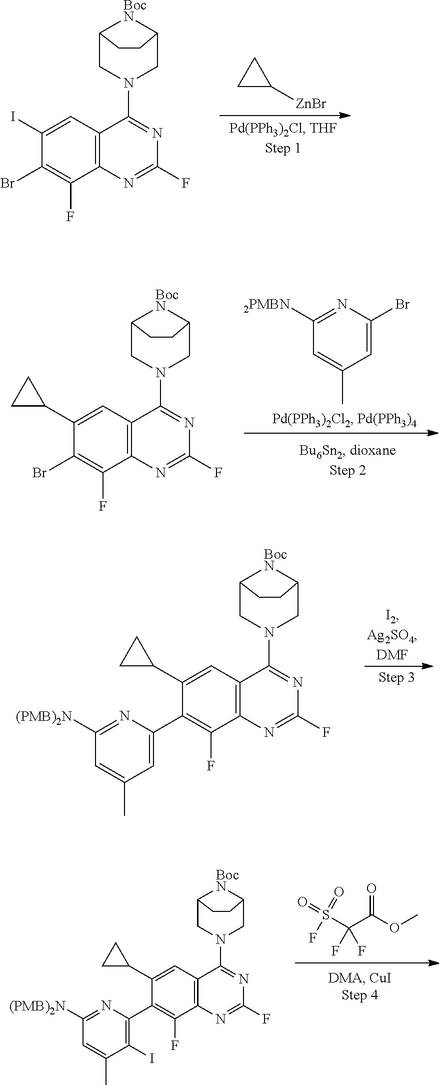

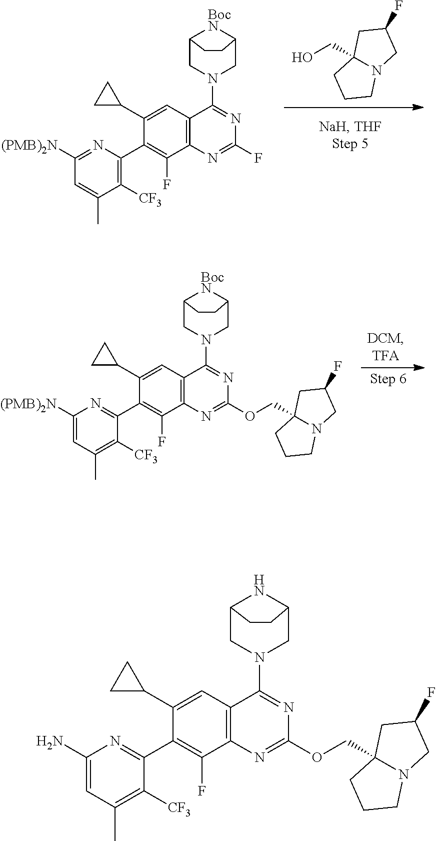

### Step 1: tert-butyl 3-(7-bromo-6-cyclopropyl-2,8-difluoroquinazolin-4-yl)-3,8-diazabicyclo[3.2.1]octane-8-carboxylate

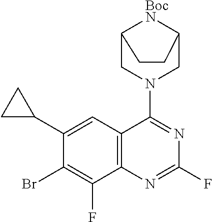 [SMILES: CC(C)(C)OC(=O)N1C2CCC1CN(c1nc(F)nc3c(F)c(Br)c(C4CC4)cc13)C2]

To a solution of tert-butyl 3-(7-bromo-2,8-difluoro-6-iodoquinazolin-4-yl)-3,8-diazabicyclo[3.2.1]octane-8-carboxylate (2.9 g, 5.0 mmol) and Pd(PPh₃)₂Cl (350 mg, 0.5 mmol) in THF (50 mL) was added cyclopropylzinc(II) bromide (12 mL, 6.0 mmol) at room temperature. The reaction mixture was stirred for 2 hours at 65° C. After completion, the reaction was directly concentrated to give the residue, the residue was purified by silica gel column chromatography, eluted with 0-30% EA in PE to give the title product (1.48 g, 3.0 mmol). MS (ESI, m/e) [M+H]⁺ 495.1, 497.1.

### Step 2: tert-butyl 3-(7-(6-(bis(4-methoxybenzyl)amino)-4-methylpyridin-2-yl)-6-cyclopropyl-2,8-difluoroquinazolin-4-yl)-3,8-diazabicyclo[3.2.1]octane-8-carboxylate

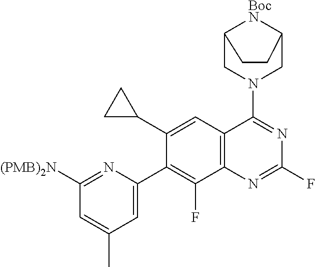

A mixture of 4-bromo-N,N-bis(4-methoxybenzyl)-6-methylpyrimidin-2-amine (1.28 g, 3 mmol), tert-butyl 3-(7-bromo-6-cyclopropyl-2,8-difluoroquinazolin-4-yl)-3,8-diazabicyclo[3.2.1]octane-8-carboxylate (988.2 mg, 2.0 mmol), Pd(PPh₃)₄ (230.8 mg, 0.2 mmol), Pd(PPh₃)₂Cl₂ (140.0 mg, 0.2 mmol) and Sn₂(ⁿBu)₆ (1.75 g, 3 mmol) in dioxane (20 mL) was stirred for 16 hours at 110° C. After completion, the resulting mixture was directly concentrated. Then the crude product was further purified by silica column (eluting with PE/EtOAc=3/2) to give the title product (400 mg, 0.52 mmol). MS (ESI, m/e) [M+1]⁺763.4.

### Step 3: tert-butyl 3-(7-(6-(bis(4-methoxybenzyl)amino)-3-iodo-4-methylpyridin-2-yl)-6-cyclopropyl-2,8-difluoroquinazolin-4-yl)-3,8-diazabicyclo [3.2.1]octane-8-carboxylate

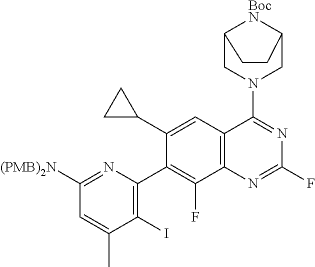

A mixture of tert-butyl 3-(7-(6-(bis(4-methoxybenzyl)amino)-4-methylpyridin-2-yl)-6-cyclopropyl-2,8-difluoroquinazolin-4-yl)-3,8-diazabicyclo[3.2.1]octane-8-carboxylate (400 mg, 0.52 mmol), Ag₂SO₄ (370 mg, 1.2 mmol) and I₂ (310 mg, 1.2 mmol) in DMF (10 mL) was stirred for 1 hour at room temperature. After completion, the resulting mixture was directly poured into water and extracted with EtOAc. Then, the crude product was further purified by silical gel column to give the title product (300 mg, 0.34 mmol). MS (ESI, m/e) [M+H]⁺ 889.3.

### Step 4: tert-butyl 3-(7-(6-(bis(4-methoxybenzyl)amino)-4-methyl-3-(trifluoro methyl)pyridin-2-yl)-6-cyclopropyl-2,8-difluoroquinazolin-4-yl)-3,8-diazabicyclo[3.2.1]octane-8-carboxylate

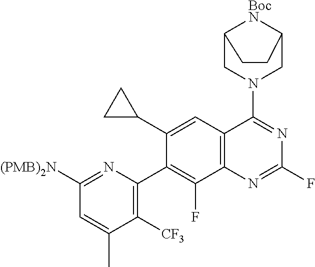

A mixture of tert-butyl 3-(7-(6-(bis(4-methoxybenzyl)amino)-3-iodo-4-methyl pyridin-2-yl)-6-cyclopropyl-2,8-difluoroquinazolin-4-yl)-3,8-diazabicyclo[3.2.1]octane-8-carboxylate (88.9 mg, 0.1 mmol), CuI (95 mg, 0.5 mmol) and methyl 2,2-difluoro-2-(fluoro sulfonyl) acetate (192 mg, 1 mmol) in DMA (10 mL) was stirred for 2 hours at 90° C. After completion, the resulting mixture was directly poured into water and extracted with EtOAc. After concentrated in vacuo, the crude product was further purified by silica column to give the title product (60 mg, 0.07 mmol). MS (ESI, m/e) [M+H]⁺ 831.4.

### Step 5: tert-butyl 3-(7-(6-(bis(4-methoxybenzyl)amino)-4-methyl-3-(trifluoro methyl)pyridin-2-yl)-6-cyclopropyl-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate

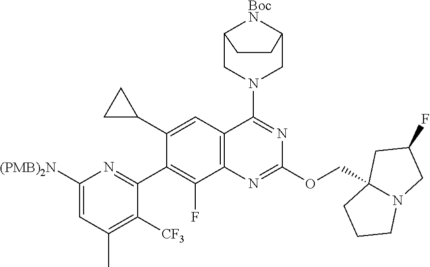

To a solution of ((2S,4R)-4-fluoro-1-methylpyrrolidin-2-yl)methanol (48 mg, 0.3 mmol) in THF (10 mL) was added NaH (24.0 mg, 0.6 mmol) and the mixture was stirred for 30 mins at room temperature. Then, tert-butyl 3-(7-(6-(bis(4-methoxybenzyl)amino)-4-methyl-3-(trifluoromethyl)pyridin-2-yl)-6-cyclopropyl-2,8-difluoroquinazolin-4-yl)-3,8-diazabicyclo [3.2.1]octane-8-carboxylate (60 mg, 0.07 mmol) was added. The reaction mixture was stirred for 2 hours at room temperature. After completion, the reaction was quenched by H₂O. Solvent was evaporated and the crude product was purified by silica column (eluting with DCM/MeOH=10/1) to give the title product (40 mg, 0.04 mmol). MS (ESI, m/e) [M+H]⁺ 970.5.

### Step 6: 6-(4-(3,8-diazabicyclo[3.2.1]octan-3-yl)-6-cyclopropyl-8-fluoro-2-(((2R,7aS)-2-fluorotetrahydro-1H-pyrrolizin-7a(5H)-yl)methoxy)quinazolin-7-yl)-4-methyl-5-(trifluoromethyl)pyridin-2-amine

To a solution of tert-butyl 3-(7-(6-(bis(4-methoxybenzyl)amino)-4-methyl-3-(trifluoromethyl)pyridin-2-yl)-6-cyclopropyl-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (40 mg, 0.04 mmol) in TFA (4 mL) was stirred for 8 hours at 40° C. After completion, the resulting mixture was directly concentrated and the residue was adjusted to pH=9 with DIPEA. Then the residues were purified by C18 column (eluting with H₂O/CH₃CN=3/1) to afford the title product (4.02 mg, FA salt). ¹H NMR (500 MHz, DMSO-d₆) δ 7.14 (s, 1H), 6.76 (s, 2H), 6.46 (s, 1H), 5.32-5.21 (m, 1H), 4.24-4.21 (m, 1H), 4.13-4.10 (m, 1H), 4.08-4.04 (m, 1H), 3.98-3.94 (m, 1H), 3.44-3.41 (m, 3H), 3.09-3.02 (m, 5H), 2.84-2.80 (m, 2H), 2.37 (s, 3H), 2.12-1.99 (m, 4H), 1.74-1.62 (m, 5H), 1.57-1.50 (m, 1H), 0.79-0.73 (m, 3H), 0.53-0.50 (s, 1H). MS (ESI, m/e) [M+H]⁺ 630.5.

### Example 130: 6-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-4-chloro-5-(1,1-difluoroethyl)pyridin-2-amine

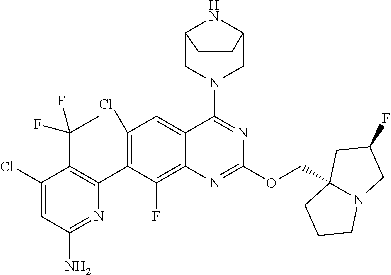 [SMILES: CC(F)(F)c1c(Cl)cc(N)nc1-c1c(Cl)cc2c(N3CC4CCC(C3)N4)nc(OC[C@@]34CCCN3C[C@H](F)C4)nc2c1F]

Synthetic Route

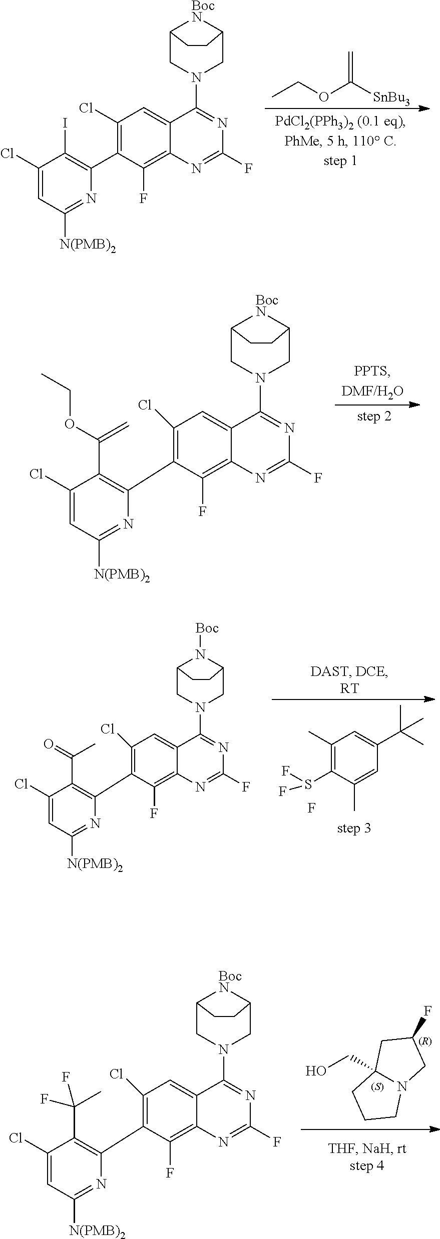

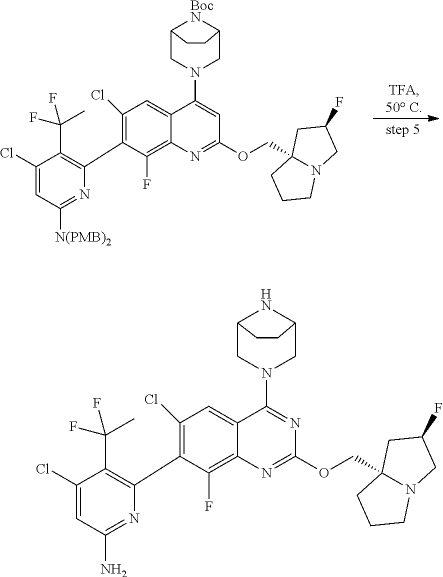

### Step 1: tert-butyl 3-(7-(6-(bis(4-methoxybenzyl)amino)-4-chloro-3-(1-ethoxy vinyl)pyridin-2-yl)-6-chloro-2,8-difluoroquinazolin-4-yl)-3,8-diazabicyclo[3.2.1]octane-8-carboxylate

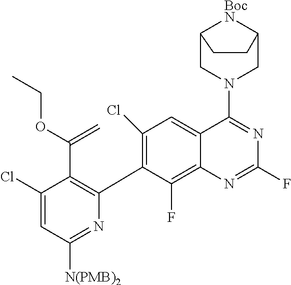

To a solution of tert-butyl 3-(7-(6-(bis(4-methoxybenzyl)amino)-4-chloro-3-iodopyridin-2-yl)-6-chloro-2,8-difluoroquinazolin-4-yl)-3,8-diazabicyclo[3.2.1]octane-8-carboxylate (1.00 g, 1.11 mmol) in Toluene (30 mL) was added tributyl(1-ethoxyvinyl)stannane (600 mg, 1.66 mmol) and PdCl₂(PPh₃)₂ (77.00 mg, 0.11 mmol). The resulting mixture was stirred for 2 h at 110° C. The resulting mixture was concentrated under reduced pressure. The residue was purified by Prep-TLC (PE/EA=4:1) to give the title product (200 mg, 21.34% yield). MS (ESI, m/e) [M+H]⁺ 847.

### Step 2: tert-butyl 3-(7-(3-acetyl-6-(bis(4-methoxybenzyl)amino)-4-chloropyridin-2-yl)-6-chloro-2,8-difluoroquinazolin-4-yl)-3,8-diazabicyclo[3.2.1]octane-8-carboxylate

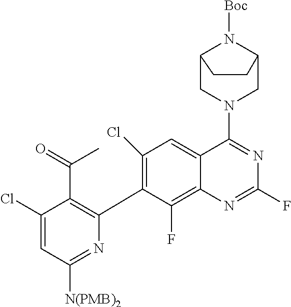

To a solution of tert-butyl 3-(7-(6-(bis(4-methoxy benzyl)amino)-4-chloro-3-(1-ethoxyvinyl)pyridin-2-yl)-6-chloro-2,8-difluoroquinazolin-4-yl)-3,8-diazabicyclo[3.2.1]octane-8-carboxylate (300 mg, 0.354 mmol) in DMF/H₂O (5 mL/2 mL) was added PPTS (890 mg, 3.54 mmol) at rt and stirred for overnight. The reaction was quenched with H₂O. The resulting mixture was extracted with EA. The combined organic layers were washed with brine, dried over anhydrous Na₂SO₄ and concentrated under reduced pressure. The residue was purified by Prep-TLC (PE/EA=3:2) to give the title product (85 mg, 29.3% yield). MS (ESI, m/e) [M+H]⁺ 819.15.

### Step 3: tert-butyl 3-(7-(6-(bis(4-methoxybenzyl)amino)-4-chloro-3-(1,1-difluoro ethyl)pyridin-2-yl)-6-chloro-2,8-difluoroquinazolin-4-yl)-3,8-diazabicyclo[3.2.1]octane-8-carboxylate

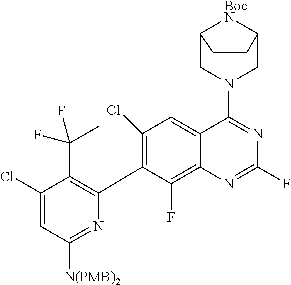

To a Teflon test tube of tert-butyl 3-(7-(3-acetyl-6-(bis(4-methoxybenzyl)amino)-4-chloropyridin-2-yl)-6-chloro-2,8-difluoroquinazolin-4-yl)-3,8-diazabicyclo[3.2.1]octane-8-carboxylate (85 mg, 0.103 mmol) in DAST (1 mL). The resulting mixture was stirred for 2 h at 90° C. in a microwave. The resulting mixture was quenched with ice-water, extracted with EA, washed with brine, dried over anhydrous Na₂SO₄ and concentrated under reduced pressure. The residue was purified by Prep-TLC (DCM/MeOH=15:1) to the title product (20 mg, 23% yield). MS (ESI, m/e) [M+H]⁺ 841.05.

### Step 4: tert-butyl 3-(7-(6-(bis(4-methoxybenzyl)amino)-4-chloro-3-(1,1-difluoro ethyl)pyridin-2-yl)-6-chloro-8-fluoro-2-(((2R,7aS)-2-fluorotetrahydro-1H-pyrrolizin-7a(5H)-yl)methoxy)quinolin-4-yl)-3,8-diazabicyclo[3.2.1]octane-8-carboxylate

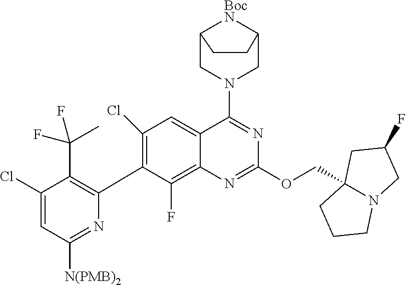

To a solution of (2R,7aS)-2-fluorotetrahydro-1H-pyrrolizin-7a(5H)-yl)methanol (7.5 mg, 0.048 mmol) in THF (extra dry, 2 mL) was added NaH (3.8 mg, 0.095 mmol, 60% purity) at rt for 30 min. Then, tert-butyl 3-(7-(6-(bis(4-methoxybenzyl)amino)-4-chloro-3-(1,1-difluoroethyl)pyridin-2-yl)-6-chloro-2,8-difluoroquinazolin-4-yl)-3,8-diazabicyclo[3.2.1]octane-8-carboxylate (20 mg, 0.024 mmol) was added in it at rt and stirred for 2 h. The resulting solution was diluted with 15 mL of H₂O, extracted with 25 mL ethyl acetate, washed with 3×15 ml brine, concentrated under vacuum. The crude product was purified by Prep-TLC (DCM:MeOH=10:1; Detector, 254/280 nm) to give the title product (20 mg, 87%). MS (ESI, m/e) [M+H]⁺ 980.15.

### Step 5: 6-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-4-chloro-5-(1,1-difluoroethyl)pyridin-2-amine

A solution of tert-butyl 3-(7-(6-(bis(4-methoxybenzyl)amino)-4-chloro-3-(1,1-difluoroethyl)pyridin-2-yl)-6-chloro-8-fluoro-2-(((2R,7aS)-2-fluorotetrahydro-1H-pyrrolizin-7a(5H)-yl)methoxy)quinolin-4-yl)-3,8-diazabicyclo[3.2.1]octane-8-carboxylate (20 mg, 0.02 mmol) in TFA (2 mL) was stirred for 2 h at 50° C. The resulting mixture was concentrated under reduced pressure. The residue was purified by Prep-HPLC to give the title product (1.5 mg, 6% yield). 1H NMR (400 MHz, CD₃OD) δ 7.90 (s, 1H), 6.82 (s, 1H), 5.64 (s, 1H), 4.66 (s, 4H), 4.23 (s, 2H), 4.08-3.78 (m, 5H), 3.64 (s, 1H), 3.38 (d, J=12.4 Hz, 1H), 2.68-2.52 (m, 2H), 2.38 (d, J=31.6 Hz, 3H), 2.12 (s, 4H), 1.95 (t, J=18.3 Hz, 3H). MS (ESI, m/e) [M+H]⁺ 640.0.

### Example 131: 4-(4-(3,8-diazabicyclo[3.2.1]octan-3-yl)-8-fluoro-2-((3-(methoxy methyl)tetrahydro-1H-pyrrolizin-7a(5H)-yl)methoxy)-6-(trifluoromethyl)quinazolin-7-yl)-7-fluorobenzo[d]thiazol-2-amine

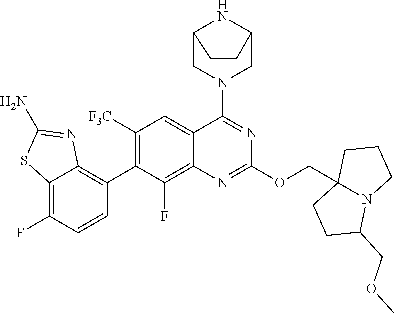 [SMILES: COCC1CCC2(COc3nc(N4CC5CCC(C4)N5)c4cc(C(F)(F)F)c(-c5ccc(F)c6sc(N)nc56)c(F)c4n3)CCCN12]

Synthetic Route

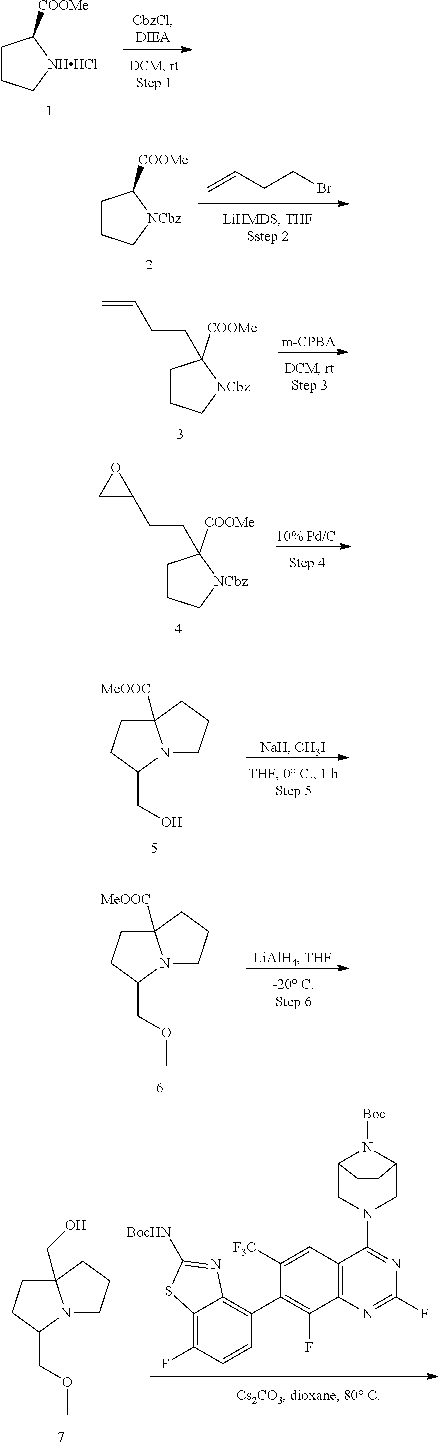

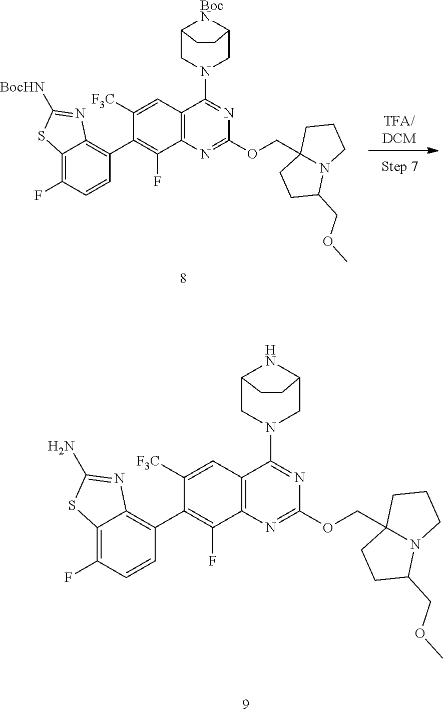 [SMILES: COCC1CCC2(COc3nc(N4CC5CCC(C4)N5C(=O)OC(C)(C)C)c4cc(C(F)(F)F)c(-c5ccc(F)c6sc(NC(=O)OC(C)(C)C)nc56)c(F)c4n3)CCCN12 ; COCC1CCC2(COc3nc(N4CC5CCC(C4)N5)c4cc(C(F)(F)F)c(-c5ccc(F)c6sc(N)nc56)c(F)c4n3)CCCN12]

### Step 1: 1-benzyl 2-methyl (S)-pyrrolidine-1,2-dicarboxylate

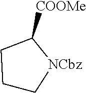 [SMILES: COC(=O)[C@@H]1CCCN1C(=O)OCc1ccccc1]

To a solution of methyl L-prolinate hydrochloride (10.0 g, 60.4 mmol) in DCM (200 mL) was added DIEA (23.4 g, 181 mmol). Then, to the above mixture was added benzyl carbonochloridate (12.4 g, 72.5 mmol) dropwise at 0° C. The reaction mixture was stirred for 3 h at room temperature. The resulting mixture was concentrated and purified by flash column chromatography (eluting with PE/EA=5/1) to give the title product (12.8 g, 81%). MS (ESI, m/e) [M+H]⁺ 264.2.

### Step 2: 1-benzyl 2-methyl 2-but-3-en-1-yl)pyrrolidine-1,2-dicarboxylate

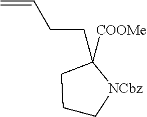 [SMILES: C=CCCC1(C(=O)OC)CCCN1C(=O)OCc1ccccc1]

To a solution of 1-benzyl 2-methyl (S)-pyrrolidine-1,2-dicarboxylate (5.00 g, 19.0 mmol) in THF (10 mL) was added dropwise a solution of LiHMDS (1 M, 22.8 mL, 1.2 eq) at −78° C. The mixture was allowed to stir at this temperature for 1 h prior to the addition of 4-bromobut-1-ene (5.13 g, 38.0 mmol). The mixture was stirred at 25° C. for 12 h. Then, the mixture was quenched by addition of saturated aq NH₄Cl (25.0 mL) and extracted with ethyl acetate (3×50 mL). The combined organic layer was washed with brine (2×25 mL), dried over anhydrous sodium sulfate and filtered. The filtrate was concentrated under reduced pressure to provide the crude residue. The residue was purified by flash column chromatography (eluting PE/EA=2:1) to give the title product (4.25 g, 70%). MS (ESI, m/e) [M+H]⁺ 318.3.

### Step 3: 1-benzyl 2-methyl 2-(2-(oxiran-2-yl)ethyl)pyrrolidine-1,2-dicarboxylate

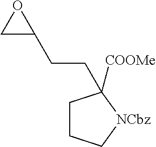 [SMILES: COC(=O)C1(CCC2CO2)CCCN1C(=O)OCc1ccccc1]

To a solution of 1-benzyl 2-methyl 2-(but-3-en-1-yl)pyrrolidine-1,2-dicarboxylate (4.00 g, 12.6 mmol) in DCM (50.0 mL) was added m-CPBA (5.44 g, 31.5 mmol, 80%). The resulting mixture was stirred for 2 hours at room temperature. The reaction was quenched by the addition of saturated aq Na₂S₂O₃ (35.0 mL). The aqueous layer was extracted with DCM (3×50 mL). The combined organic layer was washed with brine (2×30 mL), dried over anhydrous sodium sulfate and filtered. The filtrate was concentrated and purified by flash column chromatography (PE/EA=2/1) to give the title product (3.36 g, 80%). MS (ESI, m/e) [M+H]⁺ 334.3.

### Step 4: methyl 3-(hydroxymethyl)tetrahydro-1H-pyrrolizine-7a(5H)-carboxylate

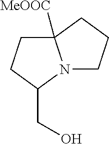 [SMILES: COC(=O)C12CCCN1C(CO)CC2]

To a solution of 1-benzyl 2-methyl 2-(2-(oxiran-2-yl)ethyl)pyrrolidine-1,2-dicarboxylate (3.36 g, 10.1 mmol) in methanol (30.0 mL) was added 10% Pd/C (0.34 g, 10.1 mmol). The suspension was desgassed by H₂ for several times. The mixture was stirred for 4 hours at room temperature under H₂ balloon. After completion, the mixture was filtered. The filtrate was concentrated under reduced pressure to provide the crude residue. The residue was purified by SiO₂ column chromatography (DCM/MeOH eluting from 100:1 to 10:1, 0.1% NEt₃) to afford the title product (605 mg, 30%). MS (ESI, m/e) [M+H]⁺ 200.3.

### Step 5: methyl 3-(methoxymethyl)tetrahydro-1H-pyrrolizine-7a(5H)-carboxylate

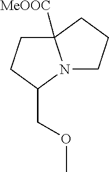

To a solution of methyl 3-(hydroxymethyl)tetrahydro-1H-pyrrolizine-7a(5H)-carboxylate (320 mg, 1.60 mmol) in THF (5.00 mL) was added NaH (128 mg, 3.20 mmol, 60%) at 0° C., the resulting mixture was stirred for 15 mins at this temperature. Then, to the above mixture was added CH₃I (273 mg, 1.92 mmol) dropwise and the mixture was stirred for another 1 hour at 0° C. The reaction was quenched with saturated aqueous NH₄Cl, and extracted with DCM (3×50 mL). The combined organic layer was washed with brine (2×30 mL), dried over anhydrous sodium sulfate and filtered. The filtrate was concentrated and purified by SiO₂ column chromatography (DCM/MeOH eluting from 100:1 to 50:1, 0.1% NEt₃) to afford the title product. MS (ESI, m/e) [M+1]⁺214.3.

### Step 6: (3-(methoxymethyl)tetrahydro-1H-pyrrolizin-7a(5H)-yl)methanol

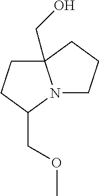

To a mixture of LiAlH₄ (99.0 mg, 2.60 mmol) in THF (7.00 mL) was added the solution of methyl 3-(methoxymethyl)tetrahydro-1H-pyrrolizine-7a(5H)-carboxylate (280 mg, 1.30 mmol) in THF (3.0 mL) dropwise at −20° C. The resulting mixture was stirred for 1 hour at this temperature. The reaction was quenched with water (0.1 mL), 15% NaOH (0.1 mL) and water (0.3 mL) at 0° C. The mixture was filtered and washed with EA (50 mL). The combined filtrate was concentrated under reduced pressure to provide the crude product. The residue was purified by SiO₂ column chromatography (DCM/MeOH eluting from 100:1 to 10:1, 0.1% NEt₃) to afford the title product (180 mg, 75%). MS (ESI, m/e) [M+H]⁺ 186.3.

### Step 7: tert-butyl 3-(7-(2-((tert-butoxycarbonyl)amino)-7-fluorobenzo[d]thiazol-4-yl)-8-fluoro-2-((3-(methoxymethyl)tetrahydro-1H-pyrrolizin-7a(5H)-yl)methoxy)-6-(trifluoro methyl)quinazolin-4-yl)-3,8-diazabicyclo[3.2.1]octane-8-carboxylate

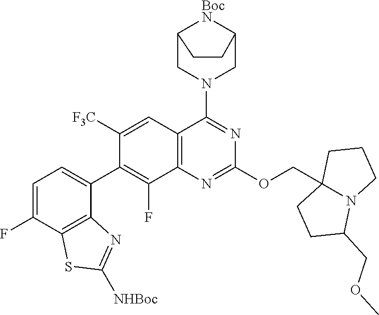 [SMILES: COCC1CCC2(COc3nc(N4CC5CCC(C4)N5C(=O)OC(C)(C)C)c4cc(C(F)(F)F)c(-c5ccc(F)c6sc(NC(=O)OC(C)(C)C)nc56)c(F)c4n3)CCCN12]

To a solution of tert-butyl 3-(7-(2-((tert-butoxycarbonyl)amino)-7-fluorobenzo[d]thiazol-4-yl)-2,8-difluoro-6-(trifluoromethyl)quinazolin-4-yl)-3,8-diazabicyclo[3.2.1]octane-8-carboxylate (35 mg, 0.05 mmol) in dioxane (1.5 mL) was added (3-(methoxymethyl)tetrahydro-1H-pyrrolizin-7a(5H)-yl)methanol (19 mg, 0.10 mmol) and Cs₂CO₃ (49 mg, 0.15 mmol). The resulting mixture was stirred for 4 hour at 80° C. The mixture was purified by prep-TLC with DCM/MeOH=20:1 to afford the title product (35 mg, 80%). MS (ESI, m/e) [M+H]⁺ 876.7.

### Step 8: 4-(4-(3,8-diazabicyclo[3.2.1]octan-3-yl)-8-fluoro-2-((3-(methoxymethyl) tetrahydro-1H-pyrrolizin-7a(5H)-yl)methoxy)-6-(trifluoromethyl)quinazolin-7-yl)-7-fluorobenzo [d]thiazol-2-amine

To a solution of tert-butyl 3-(7-(2-((tert-butoxycarbonyl)amino)-7-fluorobenzo[d]thiazol-4-yl)-8-fluoro-2-((3-(methoxymethyl)tetrahydro-1H-pyrrolizin-7a(5H)-yl)methoxy)-6-(trifluoromethyl)quinazolin-4-yl)-3,8-diazabicyclo[3.2.1]octane-8-carboxylate (35 mg, 0.04 mmol) in DCM (2.0 mL) was added TFA (1.0 mL). The resulting mixture was stirred for 2 hour at room temperature, concentrated and basified with DIEA to give a residue which was further purified by Prep-HPLC to afford the title product (12 mg, 44%). ¹H NMR (500 MHz, CD₃OD) δ 8.17 (s, 1H), 7.25-7.13 (m, 1H), 6.98 (t, J=8.4 Hz, 1H), 4.77-4.54 (m, 4H), 4.20-4.01 (m, 3H), 3.95-3.81 (m, 2H), 3.72 (ddd, J=9.9, 5.8, 3.9 Hz, 1H), 3.68-3.61 (m, 1H), 3.53 (d, J=8.1 Hz, 1H), 3.39 (t, J=9.5 Hz, 4H), 2.44-2.29 (m, 2H), 2.27-1.87 (m, 10H). MS (ESI, m/e) [M+H]⁺ 676.50.

### Example 132: 4-(4-(3,8-diazabicyclo[3.2.1]octan-3-yl)-6-chloro-8-fluoro-2-((1-((3-fluoro pyrrolidin-1-yl)methyl)cyclobutyl)methoxy)quinazolin-7-yl)-2-amino-7-fluorobenzo [b]thiophene-3-carbonitrile

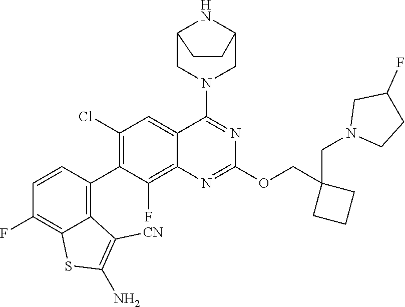 [SMILES: N#Cc1c(N)sc2c(F)ccc(-c3c(Cl)cc4c(N5CC6CCC(C5)N6)nc(OCC5(CN6CCC(F)C6)CCC5)nc4c3F)c12]

Synthetic Route

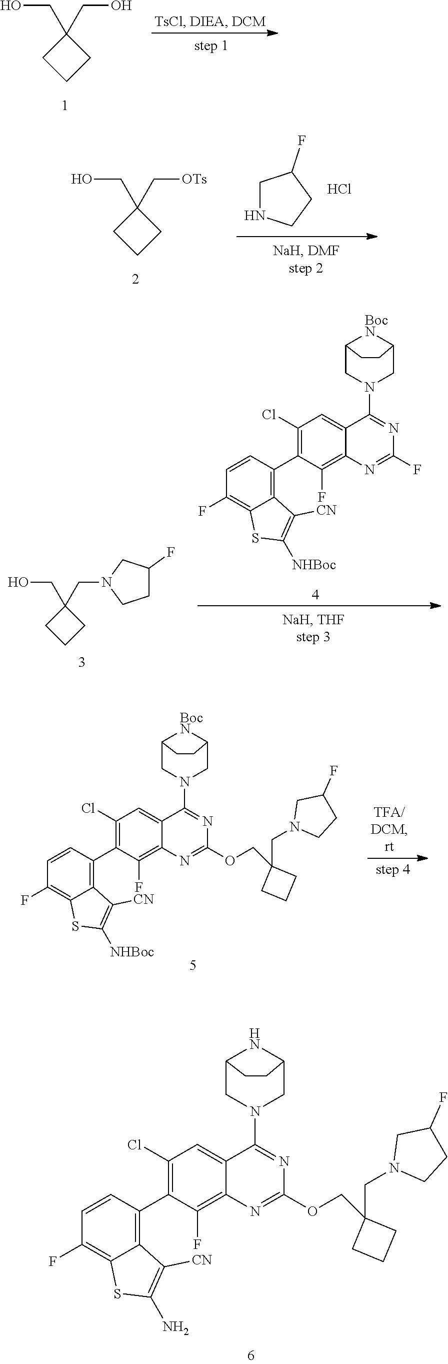

### Step 1: (1-(hydroxymethyl)cyclobutyl)methyl 4-methylbenzenesulfonate

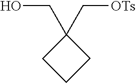 [SMILES: Cc1ccc(S(=O)(=O)OCC2(CO)CCC2)cc1]

The solution of cyclobutane-1,1-diyldimethanol (580 mg, 5.0 mmol), TsCl (950 mg, 5.0 mmol) and DIEA (1.29 g, 10 mmol) in DCM (20 mL) was stirred at 25° C. for 15 hrs. After completion, the reaction mixture was added water, extracted with DCM. The combined organic layer was dried over Na₂SO₄, filtered and concentrated. The residue was purified by Prep-TLC (PE:EA=1:1) to give the title product (900 mg, 66.7%). MS (ESI, m/e) [M+H]⁺ 271.5.

### Step 2: (1-((3-fluoropyrrolidin-1-yl)methyl)cyclobutyl)methanol

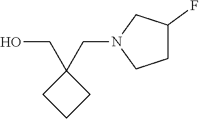 [SMILES: OCC1(CN2CCC(F)C2)CCC1]

The solution of (1-(hydroxymethyl)cyclobutyl)methyl 4-methylbenzenesulfonate (270 mg, 1.0 mmol), 3-fluoropyrrolidine, hydrogen chloride salt (126 mg, 1.0 mmol) and K₂CO₃ (420 g, 3.0 mmol) in DMF (10 mL) was stirred at 90° C. for 15 hrs under N₂ atmosphere. After completion, the reaction mixture was added water, extracted with DCM. The combined organic layer was dried over Na₂SO₄, filtered and concentrated. The residue was purified by Prep-TLC (PE:EA=1:4) to give the title product (72 mg, 38.5%). MS (ESI, m/e) [M+H]⁺ 188.4.

### Step 3: tert-butyl 3-(7-(2-((tert-butoxycarbonyl)amino)-3-cyano-7-fluorobenzo[b]thiophen-4-yl)-6-chloro-8-fluoro-2-((1-((3-fluoropyrrolidin-1-yl)methyl)cyclobutyl)methoxy) quinazolin-4-yl)-3,8-diazabicyclo[3.2.1]octane-8-carboxylate

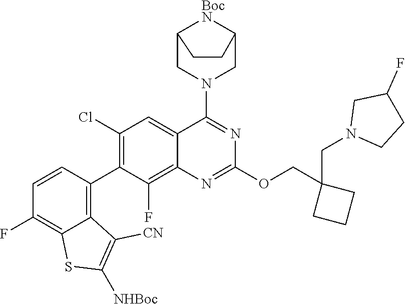 [SMILES: CC(C)(C)OC(=O)Nc1sc2c(F)ccc(-c3c(Cl)cc4c(N5CC6CCC(C5)N6C(=O)OC(C)(C)C)nc(OCC5(CN6CCC(F)C6)CCC5)nc4c3F)c2c1C#N]

To a solution of (1-((3-fluoropyrrolidin-1-yl)methyl)cyclobutyl)methanol (38 mg, 0.2 mmol) and NaH (12 mg, 0.5 mmol) in THF (2 mL) was added tert-butyl 3-(7-(2-((tert-butoxy carbonyl)amino)-3-cyano-7-fluorobenzo[b]thiophen-4-yl)-6-chloro-2,8-difluoroquinazolin-4-yl)-3,8-diazabicyclo[3.2.1]octane-8-carboxylate (70 mg, 0.1 mmol) at 0° C. The resulting mixture was stirred at room temperature for 2 hrs. After completion, the reaction mixture was added water, extracted with EA. The combined organic layer was dried over Na₂SO₄, filtered and concentrated. The residue was purified by Prep-TLC (PE:EA=1:4) to give the title product (40 mg, 46.1%). MS (ESI, m/e) [M+H]⁺ 868.5.

### Step 4: 4-(4-(3,8-diazabicyclo[3.2.1]octan-3-yl)-6-chloro-8-fluoro-2-((1-((3-fluoropyrrolidin-1-yl)methyl)cyclobutyl)methoxy)quinazolin-7-yl)-2-amino-7-fluorobenzo[b] thiophene-3-carbonitrile

To a solution of tert-butyl 3-(7-(2-((tert-butoxycarbonyl)amino)-3-cyano-7-fluoro benzo[b]thiophen-4-yl)-6-chloro-8-fluoro-2-((1-((3-fluoropyrrolidin-1-yl)methyl)cyclobutyl) methoxy)quinazolin-4-yl)-3,8-diazabicyclo[3.2.1]octane-8-carboxylate (40 mg, 0.046 mmol) in DCM (4 mL) was added TFA (2 mL) at room temperature. The resulting mixture was stirred at room temperature for 3 hrs. After completion, the reaction mixture was concentrated and the residue was purified by prep-HPLC to give the title product (0.72 mg, 2.32%). ¹H NMR (500 MHz, CD₃OD) δ 7.89 (s, 1H), 7.24-7.20 (m, 1H), 7.05-7.03 (m, 1H), 5.25-5.15 (m, 1H), 4.70-4.54 (m, 5H), 4.25-4.05 (m, 4H), 3.82-3.68 (m, 5H), 2.25-1.89 (m, 12H). MS (ESI, m/e) [M+H]⁺ 667.9.

### Example 133: 6-(4-(3,8-diazabicyclo[3.2.1]octan-3-yl)-6-chloro-8-fluoro-2-((3-(fluoromethyl)tetrahydro-1H-pyrrolizin-7a(5H)-yl)methoxy)quinazolin-7-yl)-4-methyl-5-(trifluoromethyl)pyridin-2-amine

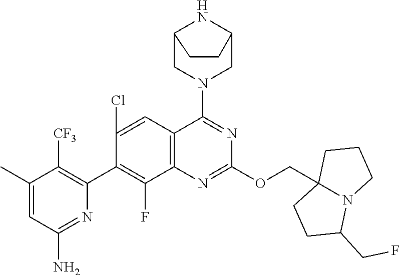 [SMILES: Cc1cc(N)nc(-c2c(Cl)cc3c(N4CC5CCC(C4)N5)nc(OCC45CCCN4C(CF)CC5)nc3c2F)c1C(F)(F)F]

Synthetic Route

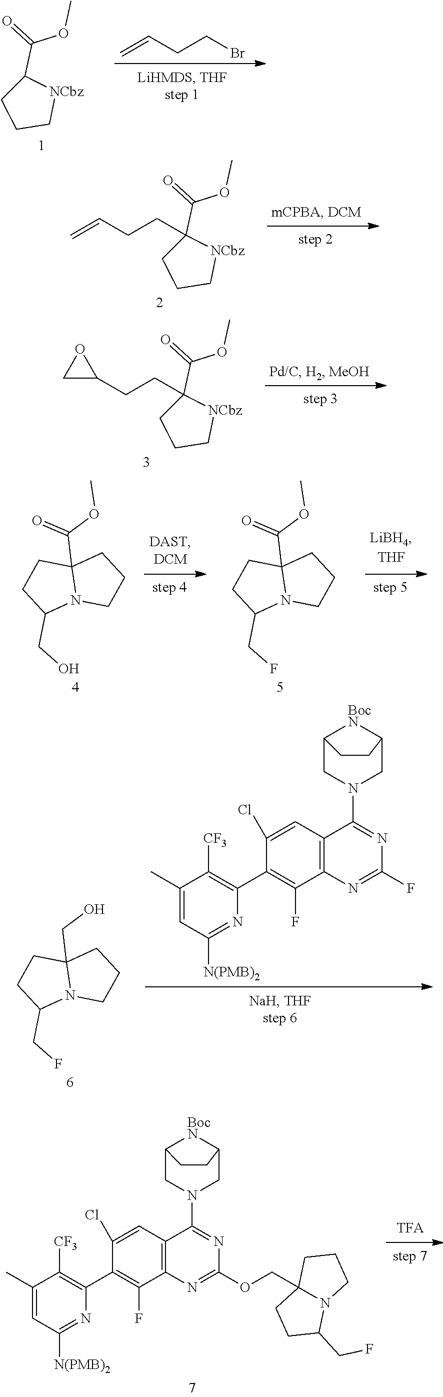

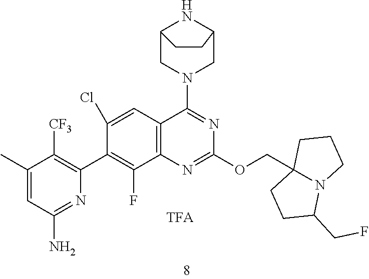 [SMILES: Cc1cc(N)nc(-c2c(Cl)cc3c(N4CC5CCC(C4)N5)nc(OCC45CCCN4C(CF)CC5)nc3c2F)c1C(F)(F)F]

### Step 1: 1-benzyl 2-methyl 2-(but-3-en-1-yl)pyrrolidine-1,2-dicarboxylate

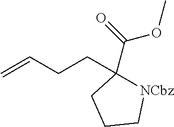 [SMILES: C=CCCC1(C(=O)OC)CCCN1C(=O)OCc1ccccc1]

A solution of 1-benzyl 2-methyl pyrrolidine-1,2-dicarboxylate (300 mg, 1.14 mmol) in THF (5 mL) was treated with LiHMDS (381.3 mg, 2.28 mmol) for 1 h at −78° C. followed by the addition of 4-bromo-1-butene (153.8 mg, 1.14 mmol) dropwise at −78° C. The resulting mixture was stirred for 2 h at room temperature. The reaction was quenched with sat. NH₄Cl (aq.) (5 mL) at 0° C. The aqueous layer was extracted with EtOAc (3×5 mL). The resulting mixture was concentrated under vacuum. The residue was purified by Prep-TLC (PE/EA=1:1) to afford the title product (200.0 mg, 55%). MS (ESI, m/e) [M+H]⁺ 317.39.

### Step 2: 1-benzyl 2-methyl 2-(2-(oxiran-2-yl)ethyl)pyrrolidine-1,2-dicarboxylate

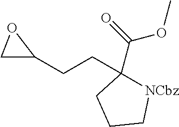 [SMILES: COC(=O)C1(CCC2CO2)CCCN1C(=O)OCc1ccccc1]

A solution of 1-benzyl 2-methyl 2-(but-3-en-1-yl)pyrrolidine-1,2-dicarboxylate (200 mg, 0.63 mmol) and m-CPBA (217.5 mg, 1.26 mmol) in DCM (2 mL) was stirred for 5 h at room temperature. The reaction was quenched with water at room temperature. The aqueous layer was extracted with DCM (3×5 mL). The organic layer was concentrated and purified by Prep-TLC (PE/EA=1:2) to afford the title product (150.0 mg, 71%). MS (ESI, m/e) [M+H]⁺ 334.15.

### Step 3: methyl 3-(hydroxymethyl)tetrahydro-1H-pyrrolizine-7a(5H)-carboxylate

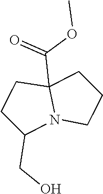

A mixture of 1-benzyl 2-methyl 2-[2-(oxiran-2-yl)ethyl]pyrrolidine-1,2-dicarboxylate (150.0 mg, 0.45 mmol) and 10% Pd/C (95.8 mg) in MeOH (2 mL) was stirred for 4 h at room temperature under hydrogen atmosphere. The resulting mixture was filtered and the filter cake was washed with MeOH (3×5 mL). The filtrate was concentrated under reduced pressure. The crude product (100.0 mg, brown oil) was used in the next step directly without further purification. MS (ESI, m/e) [M+H]⁺ 200.05.

### Step 4: methyl 3-(fluoromethyl)tetrahydro-1H-pyrrolizine-7a(5H)-carboxylate

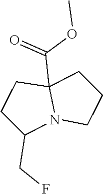

A solution of methyl 3-(hydroxymethyl)-hexahydropyrrolizine-7a-carboxylate (100.0 mg, 0.50 mmol) in THF (3 ml) was treated with DAST (404.4 mg, 2.50 mmol) at −78° C. . After stirred overnight at room temperature, the reaction was quenched by the addition of sat. Na₂CO₃ (aq.) (10 mL) at 0° C. The aqueous layer was extracted with EA (3×10 mL). The resulting mixture was concentrated under vacuum. The residue was purified by C18 column (ACN/H₂O (0.1% TFA)=0:100˜50:50) to give the title product (100.0 mg, crude). MS (ESI, m/e) [M+H]⁺ 202.00.

### Step 5: (3-(fluoromethyl)tetrahydro-1H-pyrrolizin-7a(5H)-yl)methanol

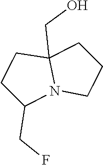 [SMILES: OCC12CCCN1C(CF)CC2]

A solution of methyl 3-(fluoromethyl)-hexahydropyrrolizine-7a-carboxylate (100.0 mg, 0.50 mmol) in MeOH/THF (4:1, 1.2 ml) was treated with LiBH₄ (10.8 mg, 0.50 mmol) at 0° C. The resulting mixture was stirred overnight at room temperature. The reaction was quenched by the addition of sat. NH₄Cl (aq.) (2 mL) at 0° C. and concentrated under vacuum. The residue was purified by C18 column (ACN/H₂O (0.1% TFA)=0:100˜50:50) to give the title product (50.0 mg, crude). MS (ESI, m/e) [M+H]⁺ 174.20.

### Step 6: tert-butyl 3-(7-(6-(bis(4-methoxybenzyl)amino)-4-methyl-3-(trifluoro methyl)pyridin-2-yl)-6-chloro-8-fluoro-2-((3-(fluoromethyl)tetrahydro-1H-pyrrolizin-7a(5H)-yl)methoxy)quinazolin-4-yl)-3,8-diazabicyclo[3.2.1]octane-8-carboxylate

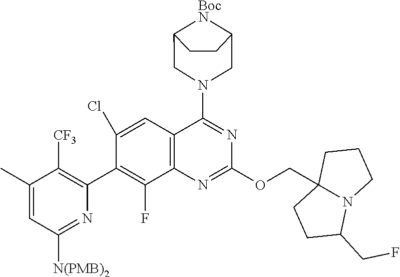

A solution of (3-(fluoromethyl)tetrahydro-1H-pyrrolizin-7a(5H)-yl)methanol (25.0 mg, 0.15 mmol) in THF (2 mL) was treated with NaH (6.9 mg, 0.30 mmol) for 1 h at 0° C. under nitrogen atmosphere followed by the addition of tert-butyl 3-(7-(6-(bis((4-methoxy phenyl)methyl)amino)-4-methyl-3-(trifluoromethyl)pyridin-2-yl)-6-chloro-2,8-difluoro quinazolin-4-yl)-3,8-diazabicyclo [3.2.1]octane-8-carboxylate (142.9 mg, 0.18 mmol) in portions at 0° C. The resulting mixture was stirred overnight at room temperature. The reaction was quenched with sat. NH₄Cl (aq.) (5 ml) at 0° C. The aqueous layer was extracted with EA (3×5 mL). The organic layer was concentrated under vacuum. The residue was purified by Prep-TLC (PE/EA=1:1) to afford the title product (25.0 mg, crude). MS (ESI, m/e) [M+H]⁺ 978.50.

### Step 7: 6-(4-(3,8-diazabicyclo[3.2.1]octan-3-yl)-6-chloro-8-fluoro-2-((3-(fluoromethyl)tetrahydro-1H-pyrrolizin-7a(5H)-yl)methoxy)quinazolin-7-yl)-4-methyl-5-(trifluoromethyl)pyridin-2-amine

A mixture of tert-butyl 3-(7-(6-(bis[(4-methoxyphenyl)methyl]amino)-4-methyl-3-(trifluoromethyl)pyridin-2-yl)-6-chloro-8-fluoro-2-((3-(fluoromethyl)-hexahydropyrrolizin-7a-yl)methoxy)quinazolin-4-yl)-3,8-diazabicyclo[3.2.1]octane-8-carboxylate (25.0 mg, 0.03 mmol) in TFA (2 mL) was stirred for 2 h at 50° C. . The residue was basified to pH 7-8 with saturated NaHCO₃ (aq.) (5 mL). The aqueous layer was extracted with EA (3×5 mL). The resulting mixture was concentrated under vacuum. The crude product was purified by Prep-HPLC to afford the title product (6.3 mg, TFA salt, 39%). ¹H NMR (300 MHz, CD₃OD) δ 7.92 (d, J=1.8 Hz, 1H), 6.65 (s, 1H), 5.15-5.01 (m, 1H), 4.69-4.50 (m, 3H), 4.24 (s, 2H), 3.89 (m, 3H), 3.74-3.57 (m, 1H), 3.54-3.33 (m, 3H), 2.46 (m, 4H), 2.37-2.24 (m, 3H), 2.23-1.89 (m, 8H). MS (ESI, m/e) [M+H-TFA]⁺638.30.

### Example 134: 4-(4-(3,8-diazabicyclo[3.2.1]octan-3-yl)-8-fluoro-2-((3-fluoro-7-(methoxy methyl)-1-azabicyclo[3.2.0]heptan-5-yl)methoxy)-6-(trifluoromethyl)quinazolin-7-yl)-7-fluorobenzo[d]thiazol-2-amine

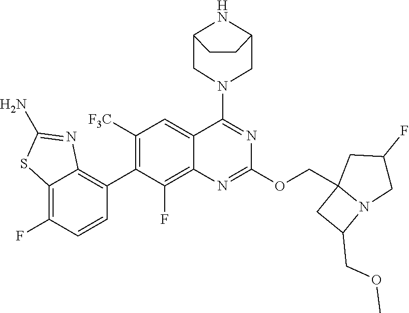 [SMILES: COCC1CC2(COc3nc(N4CC5CCC(C4)N5)c4cc(C(F)(F)F)c(-c5ccc(F)c6sc(N)nc56)c(F)c4n3)CC(F)CN12]

Synthetic Route

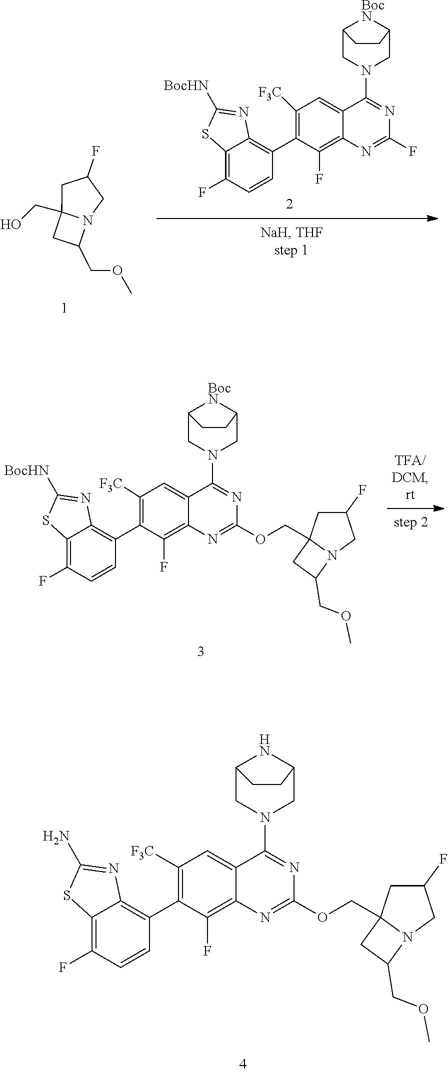 [SMILES: COCC1CC2(CO)CC(F)CN12 ; CC(C)(C)OC(=O)Nc1nc2c(-c3c(C(F)(F)F)cc4c(N5CC6CCC(C5)N6C(=O)OC(C)(C)C)nc(F)nc4c3F)ccc(F)c2s1 ; COCC1CC2(COc3nc(N4CC5CCC(C4)N5C(=O)OC(C)(C)C)c4cc(C(F)(F)F)c(-c5ccc(F)c6sc(NC(=O)OC(C)(C)C)nc56)c(F)c4n3)CC(F)CN12 ; COCC1CC2(COc3nc(N4CC5CCC(C4)N5)c4cc(C(F)(F)F)c(-c5ccc(F)c6sc(N)nc56)c(F)c4n3)CC(F)CN12]

### Step 1: tert-butyl 3-(7-(2-((tert-butoxycarbonyl)amino)-3-cyano-7-fluorobenzo [b]thiophen-4-yl)-6-chloro-8-fluoro-2-(((S)-1-(4-fluoropiperidin-1-yl)propan-2-yl)oxy) quinazolin-4-yl)-3,8-diazabicyclo[3.2.1]octane-8-carboxylate

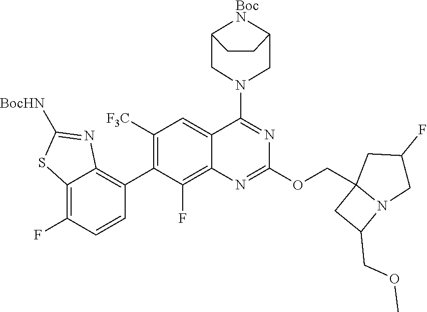 [SMILES: COCC1CC2(COc3nc(N4CC5CCC(C4)N5C(=O)OC(C)(C)C)c4cc(C(F)(F)F)c(-c5ccc(F)c6sc(NC(=O)OC(C)(C)C)nc56)c(F)c4n3)CC(F)CN12]

To a solution of (3-fluoro-7-(methoxymethyl)-1-azabicyclo[3.2.0]heptan-5-yl)methanol (38 mg, 0.2 mmol) and NaH (12 mg, 0.5 mmol) in THF (2 mL) was added tert-butyl 3-(7-(2-((tert-butoxycarbonyl)amino)-7-fluorobenzo[d]thiazol-4-yl)-2,8-difluoro-6-(trifluoromethyl)quinazolin-4-yl)-3,8-diazabicyclo[3.2.1]octane-8-carboxylate (71 mg, 0.1 mmol) at 0° C. The resulting mixture was stirred at room temperature for 2 hrs. After completion, the reaction mixture was added water and extracted with EA. The combined organic layer was dried over Na₂SO₄, filtered and concentrated. The target product was directly used next step without more purification (88.0 mg, crude). MS (ESI, m/e) [M+1]⁺880.5.

### Step 2: 4-(4-(3,8-diazabicyclo[3.2.1]octan-3-yl)-8-fluoro-2-((3-fluoro-7-(methoxy methyl)-1-azabicyclo[3.2.0]heptan-5-yl)methoxy)-6-(trifluoromethyl) quinazolin-7-yl)-7-fluoro benzo[d]thiazol-2-amine

To a solution of tert-butyl 3-(7-(2-((tert-butoxycarbonyl)amino)-3-cyano-7-fluoro benzo[b]thiophen-4-yl)-6-chloro-8-fluoro-2-(((S)-1-(4-fluoropiperidin-1-yl)propan-2-yl)oxy) quinazolin-4-yl)-3,8-diazabicyclo[3.2.1]octane-8-carboxylate (88.0 mg, 0.01 mmol) in DCM (4 mL) was added TFA (2 mL) at room temperature. The resulting mixture was stirred at room temperature for 3 hrs. After completion, the reaction mixture was concentrated and the residue was purified by prep-HPLC to give the title product (21.0 mg, FA salt, 30.9%). ¹H NMR (500 MHz, CD₃OD) δ 8.15 (s, 1H), 7.25-7.16 (m, 1H), 6.99-6.96 (m, 1H), 6.69-6.59 (m, 1H), 5.50-5.39 (m, 1H), 4.80-4.57 (m, 4H), 4.23-4.18 (m, 3H), 3.93-3.90 (m, 2H), 3.79-3.75 (m, 1H), 3.57-3.52 (m, 1H), 3.35-3.16 (m, 5H), 2.70-2.63 (m, 1H), 2.49-2.47 (m, 1H), 2.31-2.10 (m, 6H). MS (ESI, m/e) [M+H]⁺ 680.5.

### Example 135: 4-(4-(3,8-diazabicyclo[3.2.1]octan-3-yl)-6-chloro-2-(((2R,7aS)-2-fluorotetrahydro-1H-pyrrolizin-7a(5H)-yl)methoxy)quinazolin-7-yl)-5-cyclopropyl-1-methyl-1H-pyrazol-3-amine

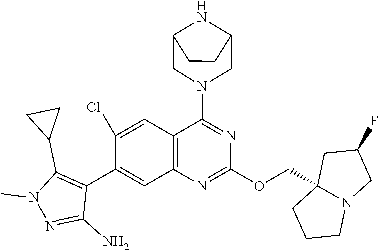 [SMILES: Cn1nc(N)c(-c2cc3nc(OC[C@@]45CCCN4C[C@H](F)C5)nc(N4CC5CCC(C4)N5)c3cc2Cl)c1C1CC1]

Synthetic Route

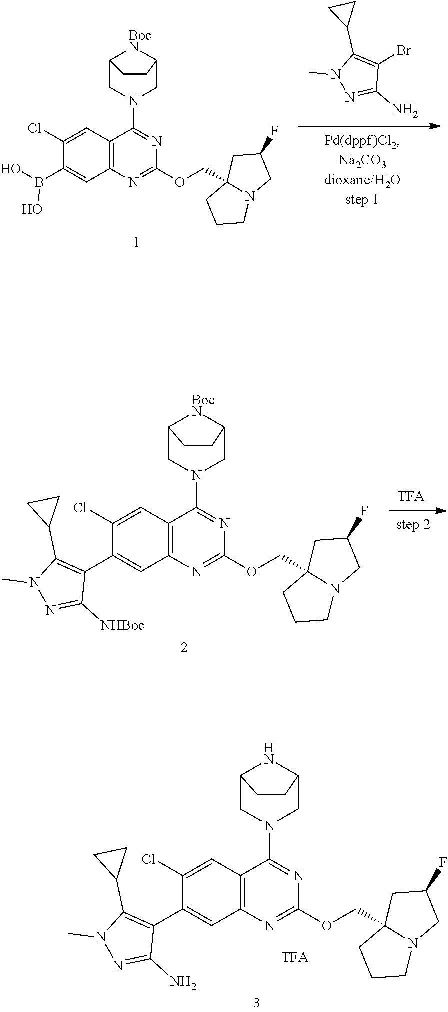 [SMILES: CC(C)(C)OC(=O)N1C2CCC1CN(c1nc(OC[C@@]34CCCN3C[C@H](F)C4)nc3cc(B(O)O)c(Cl)cc13)C2 ; Cn1nc(N)c(Br)c1C1CC1 ; Cn1nc(NC(=O)OC(C)(C)C)c(-c2cc3nc(OC[C@@]45CCCN4C[C@H](F)C5)nc(N4CC5CCC(C4)N5C(=O)OC(C)(C)C)c3cc2Cl)c1C1CC1 ; Cn1nc(N)c(-c2cc3nc(OC[C@@]45CCCN4C[C@H](F)C5)nc(N4CC5CCC(C4)N5)c3cc2Cl)c1C1CC1]

### Step 1: tert-butyl 3-(7-(3-((tert-butoxycarbonyl)amino)-5-cyclopropyl-1-methyl-1H-pyrazol-4-yl)-6-chloro-2-(((2R,7aS)-2-fluorotetrahydro-1H-pyrrolizin-7a(5H)-yl)methoxy) quinazolin-4-yl)-3,8-diazabicyclo[3.2.1]octane-8-carboxylate

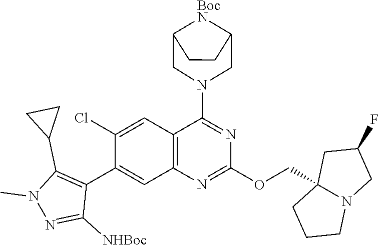 [SMILES: Cn1nc(NC(=O)OC(C)(C)C)c(-c2cc3nc(OC[C@@]45CCCN4C[C@H](F)C5)nc(N4CC5CCC(C4)N5C(=O)OC(C)(C)C)c3cc2Cl)c1C1CC1]

Into a 10 mL vial was placed 4-bromo-5-cyclopropyl-1-methyl-1H-pyrazol-3-amine (40 mg, 0.19 mmol), dioxane (1 mL), H₂O (0.2 mL), (4-(8-(tert-butoxycarbonyl)-3,8-diazabicyclo[3.2.1]octan-3-yl)-6-chloro-2-(((2R,7aS)-2-fluorotetrahydro-1H-pyrrolizin-7a(5H)-yl)methoxy)quinazolin-7-yl)boronic acid (128 mg, 0.22 mmol), Na₂CO₃ (98 mg, 0.92 mmol) and Pd(dppf)Cl₂ (15 mg, 0.02 mmol) at rt. The mixture was stirred for 2 h at 100° C. The resulting solution was diluted with 30 mL H₂O. The reaction mixture was extracted with EtOAc. The organic phase was washed with brine and concentrated under reduced pressure. The residue was purified by Prep-TLC (DCM/MeOH=10:1, Detector, 254/280 nm) to give the title product (20 mg, 16% yield). MS (ESI, m/e) [M+H]⁺ 667.35.

### Step 2: 4-(4-(3,8-diazabicyclo[3.2.1]octan-3-yl)-6-chloro-2-(((2R,7aS)-2-fluoro tetrahydro-1H-pyrrolizin-7a(5H)-yl)methoxy)quinazolin-7-yl)-5-cyclopropyl-1-methyl-1H-pyrazol-3-amine

Into a 8 Ml vial was placed tert-butyl 3-(7-(3-((tert-butoxycarbonyl)amino)-5-cyclopropyl-1-methyl-1H-pyrazol-4-yl)-6-chloro-2-(((2R,7As)-2-fluorotetrahydro-1H-pyrrolizin-7a(5H)-yl)methoxy)quinazolin-4-yl)-3,8-diazabicyclo[3.2.1]octane-8-carboxylate (20 mg, 0.03 mmol) and TFA (1 Ml). The resulting solution was stirred for 2 h at 50° C. The crude product was concentrated and purified by Prep-HPLC to give the title product (2.1 mg, TFA salt, 12.3% yield). ¹H NMR (400 MHz, CD₃OD) δ 8.11 (s, 1H), 7.68 (s, 1H), 5.72-5.48 (m, 1H), 4.67 (m, 4H), 4.23 (m, 2H), 4.00-3.80 (m, 5H), 3.65 (s, 3H), 3.48 (m, 1H), 2.79-2.53 (m, 2H), 2.50-2.35 (m, 3H), 2.12 (m, 5H), 1.66 (m, 1H), 0.85 (m, 4H). MS (ESI, m/e) [M+H]⁺ 567.20.

### Example 136: 4-(3,8-diazabicyclo[3.2.1]octan-3-yl)-6-chloro-7-(7-chloro-1H-indazol-1-yl)-2-(((2R,7aS)-2-fluorotetrahydro-1H-pyrrolizin-7a(5H)-yl)methoxy)quinazoline

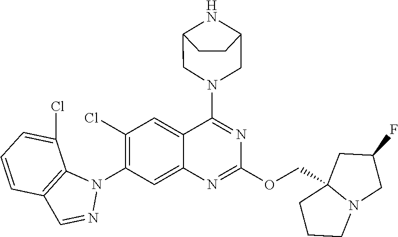 [SMILES: F[C@H]1CN2CCC[C@@]2(COc2nc(N3CC4CCC(C3)N4)c3cc(Cl)c(-n4ncc5cccc(Cl)c54)cc3n2)C1]

Synthetic Route

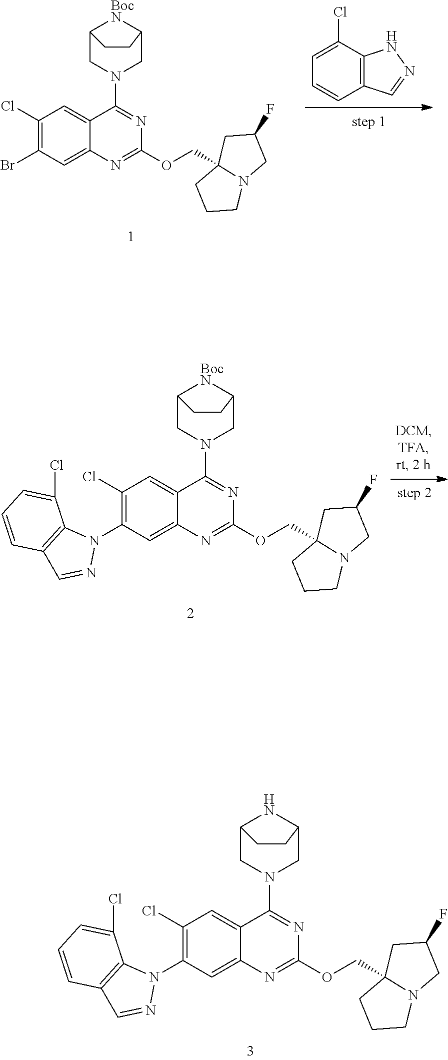 [SMILES: CC(C)(C)OC(=O)N1C2CCC1CN(c1nc(OC[C@@]34CCCN3C[C@H](F)C4)nc3cc(Br)c(Cl)cc13)C2 ; Clc1cccc2cn[nH]c12 ; CC(C)(C)OC(=O)N1C2CCC1CN(c1nc(OC[C@@]34CCCN3C[C@H](F)C4)nc3cc(-n4ncc5cccc(Cl)c54)c(Cl)cc13)C2 ; F[C@H]1CN2CCC[C@@]2(COc2nc(N3CC4CCC(C3)N4)c3cc(Cl)c(-n4ncc5cccc(Cl)c54)cc3n2)C1]

### Step 1: tert-butyl 3-(6-chloro-7-(7-chloro-1H-indazol-1-yl)-2-(((2R,7aS)-2-fluoro tetrahydro-1H-pyrrolizin-7a(5H)-yl)methoxy)quinazolin-4-yl)-3,8-diazabicyclo[3.2.1] octane-8-carboxylate

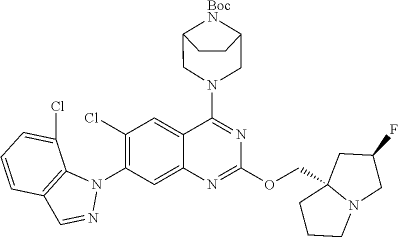 [SMILES: CC(C)(C)OC(=O)N1C2CCC1CN(c1nc(OC[C@@]34CCCN3C[C@H](F)C4)nc3cc(-n4ncc5cccc(Cl)c54)c(Cl)cc13)C2]

Into a 50-mL round-bottom flask purged and maintained with an inert atmosphere of nitrogen, was placed tert-butyl 3-(7-bromo-6-chloro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (100 mg, 0.164 mmol), DMF (2 mL), 7-chloro-1H-indazole (37 mg, 0.243 mmol), BrettPhos Pd G3 (30 mg, 0.033 mmol), BrettPhos (18 mg, 0.033 mmol) and K₂CO₃ (50 mg, 0.362 mmol). The resulting solution was stirred for overnight at 100° C. in an oil bath. Three parallel batches were combined for workup. The resulting solution was diluted with 100 mL H₂O, extracted with 100 mL ethyl acetate. The resulting mixture was washed with 3×10 mL brine. The resulting organic phase was concentrated under vacuum. The residue was purified by Prep-TLC (PE/EA=3:1) to give the title product (100 mg, 30% yield). MS (ESI, m/e) [M+H]⁺ 682.50.

### Step 2: 4-(3,8-diazabicyclo[3.2.1]octan-3-yl)-6-chloro-7-(7-chloro-1H-indazol-1-yl)-2-(((2R,7aS)-2-fluorotetrahydro-1H-pyrrolizin-7a(5H)-yl)methoxy)quinazoline

The resulting mixture of tert-butyl 3-(6-chloro-7-(7-chloro-1H-indazol-1-yl)-2-(((2R,7aS)-2-fluorotetrahydro-1H-pyrrolizin-7a(5H)-yl)methoxy)quinazolin-4-yl)-3,8-diaza bicyclo[3.2.1]octane-8-carboxylate (100 mg, 0.146 mmol) in TFA (2 ml) was stirred for 5 h at 50° C. The resulting mixture was concentrated under reduced pressure. The residue was purified by Prep-HPLC to give the title product (23 mg, trifluoroacetate salt, 27% yield). 1H NMR (300 MHz, CDCl₃) δ 8.46 (s, 1H), 8.11 (s, 1H), 7.95 (s, 1H), 7.68 (d, J=8.5 Hz, 1H), 7.40 (d, J=7.1 Hz, 1H), 7.09 (dd, J=8.5, 7.2 Hz, 1H), 5.63-5.46 (m, 1H), 4.82 (d, J=11.6 Hz, 1H), 4.55 (m, 3H), 4.21-3.90 (m, 6H), 3.61 (m, 1H), 3.31 (m, 1H), 3.00-2.74 (m, 1H), 2.74-2.59 (m, 1H), 2.58-2.44 (m, 2H), 2.40-2.22 (m, 4H), 2.15-2.03 (m, 2H). MS (ESI, m/e) [M+H]⁺ 582.10.

### Example 137: 6-(4-(3-oxa-7,9-diazabicyclo[3.3.1]nonan-9-yl)-6,8-difluoro-2-(((2R,4R)-4-fluoro-2-(methoxymethyl)-1-methylpyrrolidin-2-yl)methoxy)quinazolin-7-yl)-4-methyl-5-(trifluoromethyl)pyridin-2-amine

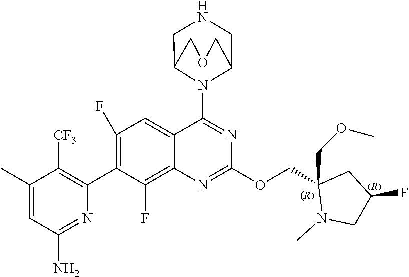

Synthetic Route

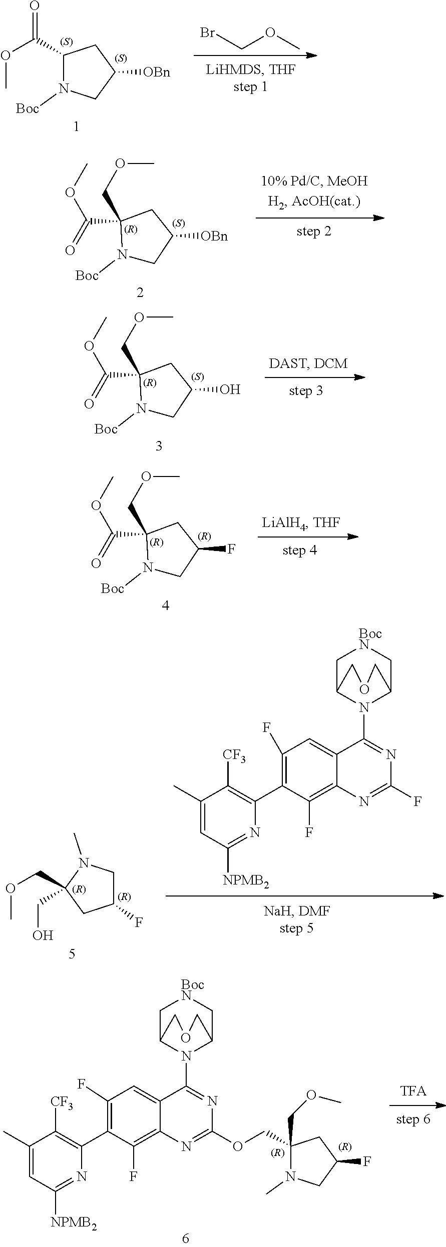

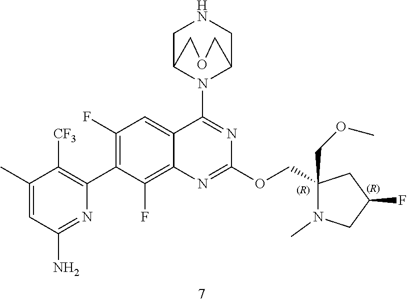

### Step 1: 1-(tert-butyl) 2-methyl (2R,4S)-4-(benzyloxy)-2-(methoxymethyl) pyrrolidine-1,2-dicarboxylate

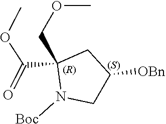

To a stirred solution of 1-tert-butyl 2-methyl (2S,4S)-4-(benzyloxy)pyrrolidine-1,2-dicarboxylate (10 g, 29.80 mmol) in THF (150 ml) was added LiHMDS (44.8 mL, 44.75 mmol) in portions at −78° C. The resulting mixture was stirred for 3 h at −40° C. To the above mixture was added bromo(methoxy)methane (5.59 g, 44.75 mmol) in portions at −78° C. The resulting mixture was stirred overnight at room temperature. The reaction was quenched with sat. NH₄Cl (aq.), extracted with EtOAc (3×300 mL). The combined organic layers were washed with brine (2×500 mL), dried over anhydrous Na₂SO₄. After filtration, the filtrate was concentrated under reduced pressure. The residue was purified by silica gel column chromatography, eluted with PE/EA (5:1) to afford the title product (6.6 g, 58%). MS (ESI, m/e) [M+H]⁺ 379.45.

### Step 2: 1-(tert-butyl) 2-methyl (2R,4S)-4-hydroxy-2-(methoxymethyl)pyrrolidine-1,2-dicarboxylate

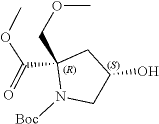

A solution of 1-tert-butyl 2-methyl (2R,4S)-4-(benzyloxy)-2-(methoxymethyl) pyrrolidine-1,2-dicarboxylate (6.3 g, 16.62 mmol), 10% Pd/C (6 g) and AcOH (20 l, cat.) in MeOH (100 ml) was stirred overnight at room temperature under hydrogen atmosphere. The resulting mixture was filtered and the filter cake was washed with MeOH (3×200 mL). The filtrate was concentrated under reduced pressure. The crude product (4.6 g) was directly used in the next step without further purification. MS (ESI, m/e) [M+H]⁺ 289.33.

### Step 3: 1-(tert-butyl) 2-methyl (2R,4R)-4-fluoro-2-(methoxymethyl)pyrrolidine-1,2-dicarboxylate

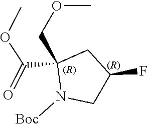

To a stirred solution of 1-tert-butyl 2-methyl (2R,4S)-4-hydroxy-2-(methoxy methyl)pyrrolidine-1,2-dicarboxylate (4.26 g, 14.68 mmol) in DCM (80 ml) was added DAST (10.6 mL, 80.74 mmol) in portions at −78° C. The reaction was quenched with sat. NH₄Cl (aq.) at room temperature, extracted with EtOAc (3×100 mL), washed with brine (100 mL) and dried over anhydrous Na₂SO₄. After filtration, the filtrate was concentrated under reduced pressure. The residue was purified by silica gel column chromatography, eluted with PE/EA (1:1) to afford the title product (2 g, 47%). MS (ESI, m/e) [M+H]⁺ 291.32.

### Step 4: ((2R,4R)-4-fluoro-2-(methoxymethyl)-1-methylpyrrolidin-2-yl)methanol

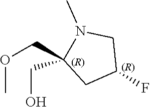

To a stirred solution of 1-tert-butyl 2-methyl (2R,4R)-4-fluoro-2-(methoxymethyl) pyrrolidine-1,2-dicarboxylate (1.2 g, 4.11 mmol) in THF (20 ml) was added LiAlH₄ (0.63 g, 16.42 mmol) dropwise at 0° C. The resulting mixture was stirred for 2 h at room temperature. The reaction was quenched by Na₂SO₄.10H₂O at 0° C. The resulting mixture was filtered, the filter cake was washed with MeOH (3×30 mL). The filtrate was concentrated under reduced pressure. The residue was purified by silica gel column chromatography, eluted with DCM/MeOH (6:1) to afford the title product (500 mg, 68%). MS (ESI, m/e) [M+H]⁺ 177.22.

### Step 5: tert-butyl 9-(7-(6-(bis(4-methoxybenzyl)amino)-4-methyl-3-(trifluoro methyl)pyridin-2-yl)-6,8-difluoro-2-(((2R,4R)-4-fluoro-2-(methoxymethyl)-1-methylpyrrolidin-2-yl)methoxy)quinazolin-4-yl)-3-oxa-7,9-diazabicyclo[3.3.1]nonane-7-carboxylate

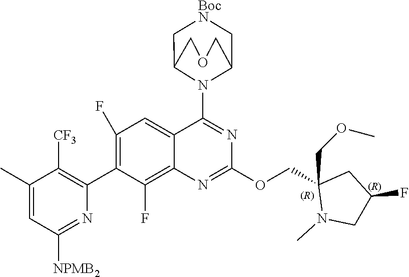

A solution of ((2R,4R)-4-fluoro-2-(methoxymethyl)-1-methylpyrrolidin-2-yl) methanol (30.0 mg, 0.17 mmol) and NaH (8.1 mg, 0.34 mmol) in THF (1.4 ml) was stirred for 30 min at room temperature. To the above mixture was added tert-butyl 9-(7-(6-(bis((4-methoxyphenyl)methyl)amino)-4-methyl-3-(trifluoromethyl)pyridin-2-yl)-2,6,8-trifluoro quinazolin-4-yl)-3-oxa-7,9-diazabicyclo[3.3.1]nonane-7-carboxylate (70 mg, 0.09 mmol) in portions at room temperature. The resulting mixture was stirred for additional 1.5 h at room temperature. The reaction was quenched with NH₄HCl (aq.) at room temperature, extracted with EtOAc (3×30 mL). The combined organic layers were washed with brine (50 mL) and dried over anhydrous Na₂SO₄. After filtration, the filtrate was concentrated under reduced pressure. The residue was purified by Prep-TLC (PE/EA 1:1) to afford the title product (50 mg, 59%). MS (ESI, m/e) [M+H]⁺ 980.04.

### Step 6: 6-(4-(3-oxa-7,9-diazabicyclo[3.3.1]nonan-9-yl)-6,8-difluoro-2-(((2R,4R)-4-fluoro-2-(methoxymethyl)-1-methylpyrrolidin-2-yl)methoxy)quinazolin-7-yl)-4-methyl-5-(trifluoromethyl)pyridin-2-amine

A solution of tert-butyl 9-(7-(6-(bis(4-methoxybenzyl)amino)-4-methyl-3-(trifluoromethyl)pyridin-2-yl)-6,8-difluoro-2-(((2R,4R)-4-fluoro-2-(methoxymethyl)-1-methyl pyrrolidin-2-yl)methoxy)quinazolin-4-yl)-3-oxa-7,9-diazabicyclo[3.3.1]nonane-7-carboxylate (50 mg, 0.05 mmol)) in TFA (1 ml) was stirred overnight at 50° C. The resulting mixture was concentrated under reduced pressure. The residue was purified by Prep-HPLC to afford the title product (10.2 mg, 31.22%). 1H NMR (400 MHz, CD₃OD) δ 7.30 (d, J=9.6 Hz, 1H), 6.50 (s, 1H), 5.14-4.95 (m, 1H), 4.42-4.30 (m, 3H), 4.26 (d, J=10.6 Hz, 1H), 4.10 (s, 4H), 3.58 (d, J=9.8 Hz, 1H), 3.46 (d, J=9.8 Hz, 1H), 3.34 (d, J=5.7 Hz, 2H), 3.27-3.19 (m, 5H), 3.07-2.89 (m, 2H), 2.45 (s, 3H), 2.38-2.32 (m, 3H), 2.25 (dd, J=15.0, 6.0 Hz, 1H), 2.14-1.98 (m, 1H). MS (ESI, m/e) [M+H]⁺ 641.25.

### Example 138: 6-(4-(3-oxa-7,9-diazabicyclo[3.3.1]nonan-9-yl)-6,8-difluoro-2-((2-fluoro-6-methoxytetrahydro-1H-pyrrolizin-7a(5H)-yl)methoxy)quinazolin-7-yl)-4-methyl-5-(trifluoromethyl)pyridin-2-amine

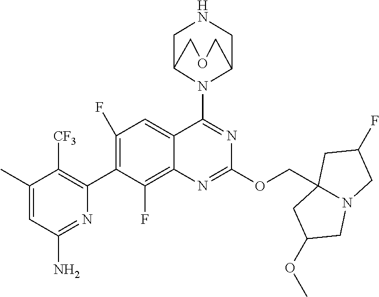 [SMILES: COC1CN2CC(F)CC2(COc2nc(N3C4CNCC3COC4)c3cc(F)c(-c4nc(N)cc(C)c4C(F)(F)F)c(F)c3n2)C1]

Synthetic Route

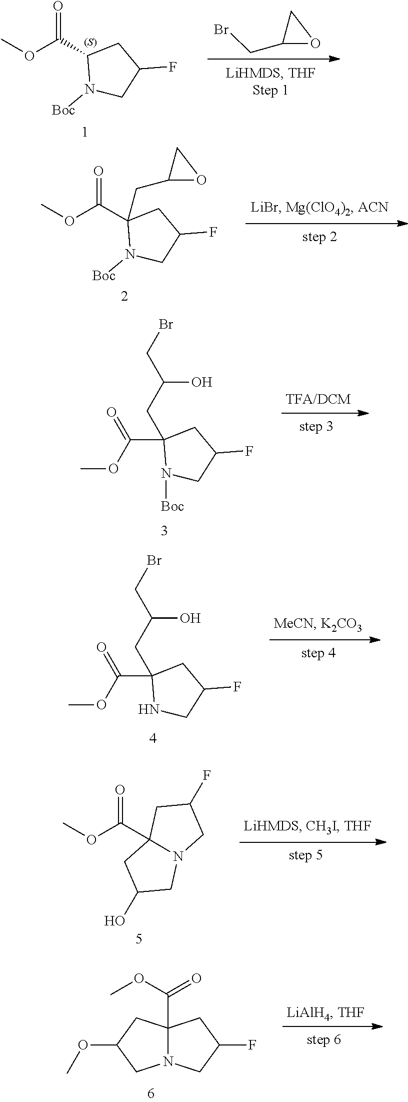

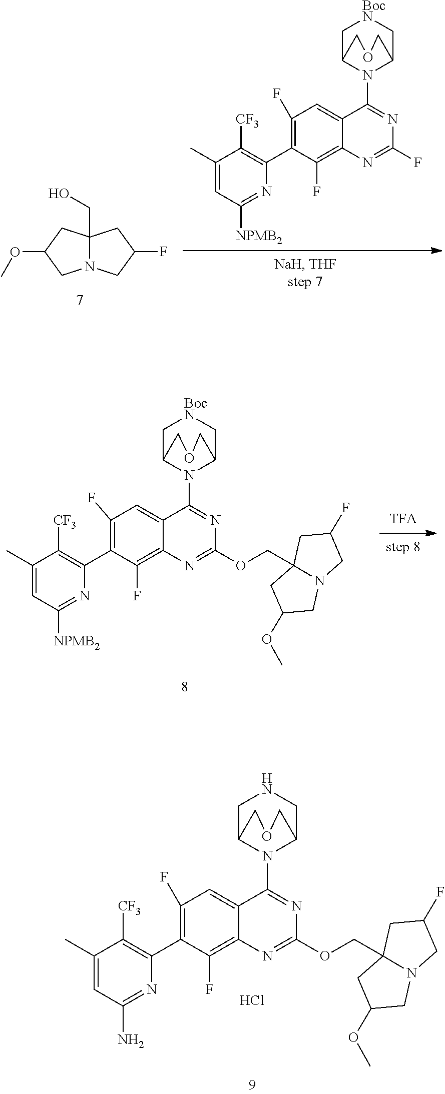

### Step 1

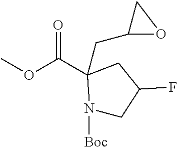 [SMILES: COC(=O)C1(CC2CO2)CC(F)CN1C(=O)OC(C)(C)C]

To a solution of 1-tert-butyl 2-methyl (2S)-4-fluoropyrrolidine-1,2-dicarboxylate (10 g, 40.44 mmol) in THF (150 mL) was added dropwise LiHMDS solution (2 M in THF, 30.33 mL) at −78° C. under N₂ atmosphere. The reaction mixture was stirred at −78° C. for 30 min. Then a solution of 2-(bromomethyl)oxirane (8.3 g, 60.66 mmol) in 15 mL THF was added dropwise and the mixture was stirred overnight at room temperature. The reaction was quenched with NH₄Cl (aq), and then the mixture was extracted with EA (2*250 mL). The combined organic extracts were washed with brine (100 mL), dried over anhydrous Na₂SO₄ and concentrated. The residue was purified by silica gel column chromatography to afford the title product (4 g, 32%). MS (ESI, m/e) [M+Na]⁺326.05.

### Step 2: 1-(tert-butyl) 2-methyl 2-(3-bromo-2-hydroxypropyl)-4-fluoropyrrolidine-1,2-dicarboxylate

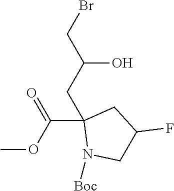 [SMILES: COC(=O)C1(CC(O)CBr)CC(F)CN1C(=O)OC(C)(C)C]

To a stirred solution of 1-tert-butyl 2-methyl 4-fluoro-2-(oxiran-2-ylmethyl) pyrrolidine-1,2-dicarboxylate (4 g, 13.18 mmol) in anhydrous CH₃CN (100 mL) at 0° C. (ice bath) was added LiBr (3.44 g, 39.56 mmol) and Mg(ClO₄)₂ (7.32 g, 32.97 mmol). The ice bath was removed and the reaction was allowed to stir at 25° C. for 1 h. The mixture was diluted with DCM and washed with 1M HCl(aq), then the aqueous phase was extracted with DCM. The combined organic extracts were washed with H₂O, dried over anhydrous Na₂SO₄ and concentrated under reduced pressure to give crude title product (3.5 g, crude) which was used in the next step without further purification.

### Step 3: methyl 2-(3-bromo-2-hydroxypropyl)-4-fluoropyrrolidine-2-carboxylate

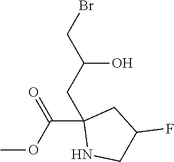

Into a 100-mL round-bottom flask was placed methyl 1-tert-butyl 2-methyl 2-(3-bromo-2-hydroxypropyl)-4-fluoropyrrolidine-1,2-dicarboxylate (3.5 g, crude), DCM (30 mL) and TFA (15 mL). The resulting solution was stirred for 1 h at room temperature. The resulting mixture was concentrated under reduced pressure to give crude title product (2.3 g) which was used in the next step without further purification. MS (ESI, m/e) [M+H]⁺ 284.05.

### Step 4: methyl 2-fluoro-6-hydroxytetrahydro-1H-pyrrolizine-7a(5H)-carboxylate

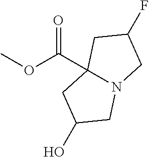

Into a 250-mL round-bottom flask was placed methyl 2-(3-bromo-2-hydroxypropyl)-4-fluoropyrrolidine-2-carboxylate (2.3 g, 8.09 mmol), K₂CO₃ (3.36 g, 24.28 mmol) and ACN (50 mL). The resulting solution was stirred for 3 h at 80° C. The resulting mixture was diluted with H₂O at 0° C. and extracted with EA. The organic layer was concentrated. The residue was applied onto a silica gel column eluted with MeOH/DCM (0:100-10:90) to give the title product (1 g, 60%). MS (ESI, m/e) [M+H]⁺ 204.15.

### Step 5: methyl 2-fluoro-6-methoxytetrahydro-1H-pyrrolizine-7a(5H)-carboxylate

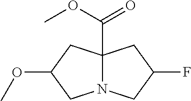

To a solution of methyl 2-fluoro-6-hydroxy-hexahydropyrrolizine-7a-carboxylate (500 mg, 2.46 mmol) in THF (10 mL) was added dropwise LiHMDS solution (2 M in THF, 1.85 mL) at 0° C. The reaction mixture was stirred at 0° C. for 30 min. Then a solution of CH₃I (523.8 mg, 3.69 mmol) in 15 mL THF was added dropwise and the mixture was stirred overnight at room temperature. The reaction was quenched with NH₄Cl (aq), and then the mixture was extracted with EA (2*50 mL). The combined organic extracts were washed with brine (10 mL), dried over anhydrous Na₂SO₄ and concentrated. The residue was purified by silica gel column chromatography to afford the title product (100 mg, 18%). MS (ESI, m/e) [M+H]⁺ 218.10.

### Step 6: (2-fluoro-6-methoxytetrahydro-1H-pyrrolizin-7a(5H)-yl)methanol

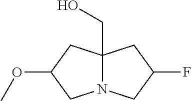

To a solution of methyl 2-fluoro-6-methoxy-hexahydropyrrolizine-7a-carboxylate (100 mg, 0.46 mmol) in THF (5 mL) was added LiAlH₄ (26.2 mg, 0.69 mmol) at 0° C. After stirred for 1 h at 0° C. The resulting mixture was quenched with Na₂SO₄10H₂O at 0° C. The resulting mixture was filtered and the filter cake was washed with MeOH. The filtrate was concentrated under reduced pressure. The residue was applied onto a silica gel column with MeOH/DCM (0:100-10:90) to give the title product (50 mg, 57%). MS (ESI, m/e) [M+H]⁺ 190.05.

### Step 7: tert-butyl 9-(7-(6-(bis(4-methoxybenzyl)amino)-4-methyl-3-(trifluoro methyl)pyridin-2-yl)-6,8-difluoro-2-((2-fluoro-6-methoxytetrahydro-1H-pyrrolizin-7a(5H)-yl)methoxy)quinazolin-4-yl)-3-oxa-7,9-diazabicyclo[3.3.1]nonane-7-carboxylate

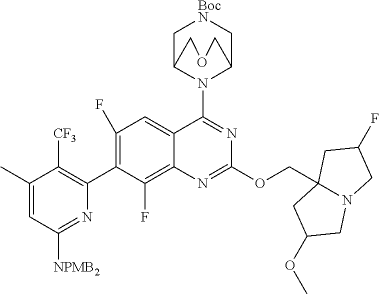

To a solution of (2-fluoro-6-methoxy-hexahydropyrrolizin-7a-yl)methanol (40 mg, 0.21 mmol) in THF (3 mL) was added NaH (16.9 mg, 0.42 mmol, 60%) at 0° C. The mixture was stirred for 15 min. Tert-butyl 9-(7-(6-(bis((4-methoxyphenyl)methyl)amino)-4-methyl-3-(trifluoromethyl) pyridin-2-yl)-2,6,8-trifluoroquinazolin-4-yl)-3-oxa-7,9-diazabicyclo [3.3.1]nonane-7-carboxylate (174.3 mg, 0.21 mmol) was added and the mixture was allowed to warm to room temperature and stirred for 2 hrs. The reaction mixture was quenched by NH₄HCl (aq) at 0° C. and extracted with EA (3*25 mL). The organic layer was concentrated and purified by Prep-TLC MeOH/DCM (1:15) to give the title product (80 mg, 38%). MS (ESI, m/e) [M+H]⁺ 994.85.

### Step 8: 6-(4-(3-oxa-7,9-diazabicyclo[3.3.1]nonan-9-yl)-6,8-difluoro-2-((2-fluoro-6-methoxytetrahydro-1H-pyrrolizin-7a(5H)-yl)methoxy)quinazolin-7-yl)-4-methyl-5-(trifluoro methyl)pyridin-2-amine

A solution of tert-butyl 9-(7-(6-(bis(4-methoxybenzyl)amino)-4-methyl-3-(trifluoromethyl)pyridin-2-yl)-6,8-difluoro-2-((2-fluoro-6-methoxytetrahydro-1H-pyrrolizin-7a(5H)-yl)methoxy)quinazolin-4-yl)-3-oxa-7,9-diazabicyclo[3.3.1]nonane-7-carboxylate (70 mg, 0.07 mmol) and TFA (2 mL) was stirred for 2 h at 50° C. . The resulting mixture was concentrated under reduced pressure. The crude product was purified by Prep-HPLC to give the title product (12.7 mg, HCl salt, 26%). ¹H NMR (400 MHz, CD₃OD) δ 7.77 (d, J=9.5 Hz, 1H), 7.13 (s, 1H), 5.63-5.50 (m, 1H), 4.95-4.92 (m, 1H), 4.85-4.83 (m, 1H), 4.43-4.35 (m, 1H), 4.35-4.20 (m, 4H), 4.16-4.00 (m, 1H), 3.91-3.70 (m, 6H), 3.70-3.55 (m, 2H), 3.38 (d, J=2.5 Hz, 3H), 2.86-2.77 (m, 1H), 2.67-2.57 (m, 5H), 2.55-2.49 (m, 2H). MS (ESI, m/e) [M+H]⁺ 654.2.

### Example 139: 4-(4-(3-oxa-7,9-diazabicyclo[3.3.1]nonan-9-yl)-2-((2,6-difluoro tetrahydro-1H-pyrrolizin-7a(5H)-yl)methoxy)-8-fluoro-6-(trifluoromethyl)quinazolin-7-yl)-7-fluorobenzo[d]thiazol-2-amine

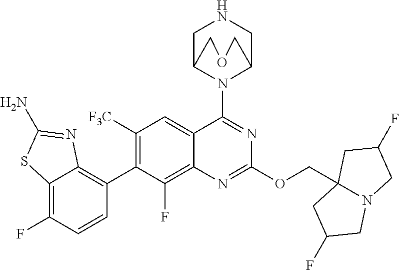 [SMILES: Nc1nc2c(-c3c(C(F)(F)F)cc4c(N5C6CNCC5COC6)nc(OCC56CC(F)CN5CC(F)C6)nc4c3F)ccc(F)c2s1]

Synthetic Route

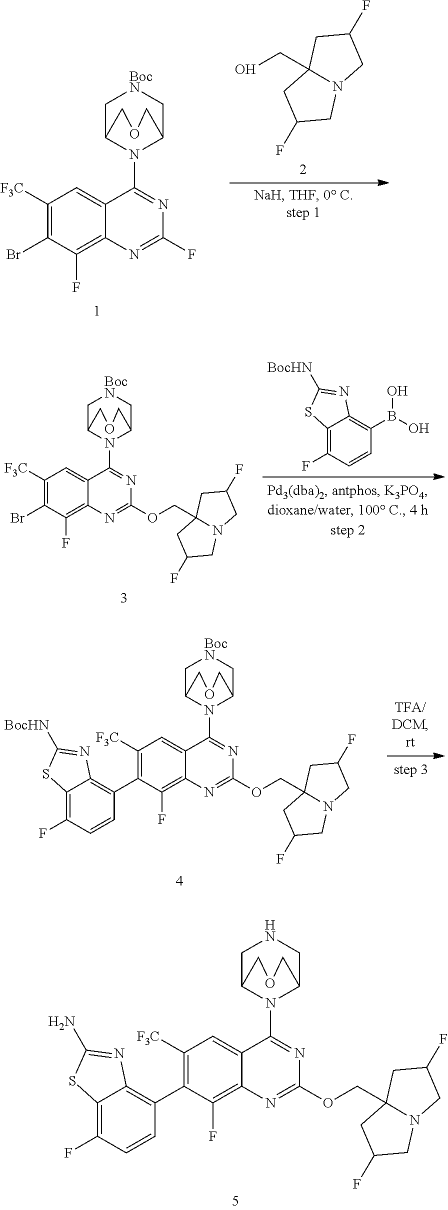 [SMILES: CC(C)(C)OC(=O)N1CC2COCC(C1)N2c1nc(F)nc2c(F)c(Br)c(C(F)(F)F)cc12 ; OCC12CC(F)CN1CC(F)C2 ; CC(C)(C)OC(=O)N1CC2COCC(C1)N2c1nc(OCC23CC(F)CN2CC(F)C3)nc2c(F)c(Br)c(C(F)(F)F)cc12 ; CC(C)(C)OC(=O)Nc1nc2c(B(O)O)ccc(F)c2s1 ; CC(C)(C)OC(=O)Nc1nc2c(-c3c(C(F)(F)F)cc4c(N5C6COCC5CN(C(=O)OC(C)(C)C)C6)nc(OCC56CC(F)CN5CC(F)C6)nc4c3F)ccc(F)c2s1 ; Nc1nc2c(-c3c(C(F)(F)F)cc4c(N5C6CNCC5COC6)nc(OCC56CC(F)CN5CC(F)C6)nc4c3F)ccc(F)c2s1]

### Step 1: tert-butyl 9-(7-bromo-2-((2,6-difluorotetrahydro-1H-pyrrolizin-7a(5H)-yl)methoxy)-8-fluoro-6-(trifluoromethyl)quinazolin-4-yl)-3-oxa-7,9-diazabicyclo[3.3.1]nonane-7-carboxylate

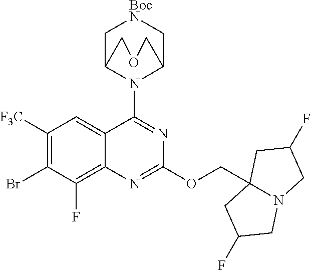 [SMILES: CC(C)(C)OC(=O)N1CC2COCC(C1)N2c1nc(OCC23CC(F)CN2CC(F)C3)nc2c(F)c(Br)c(C(F)(F)F)cc12]

To a solution of (2,6-difluorotetrahydro-1H-pyrrolizin-7a(5H)-yl)methanol (36 mg, 0.2 mmol) in THF (5 mL) was added sodium hydride (10 mg, 0.4 mmol) at room temperature. The resulting mixture was stirred at room temperature for 0.3 hrs, Then, tert-butyl 9-(7-bromo-2,8-difluoro-6-(trifluoromethyl)quinazolin-4-yl)-3-oxa-7,9-diazabicyclo[3.3.1] nona-7-carboxylate (108 mg, 0.2 mmol) was added to the reaction mixture and stirred for 2 hrs at room temperature. After completion, the reaction mixture was purified by Prep-TLC (PE:EA=1:1) to give the title product (70 mg, 50.4%). MS (ESI, m/e) [M+1]⁺696.5, 698,5.

### Step 2: tert-butyl 9-(7-(2-((tert-butoxycarbonyl)amino)-7-fluorobenzo[d]thiazol-4-yl)-2-((2,6-difluorotetrahydro-1H-pyrrolizin-7a(5H)-yl)methoxy)-8-fluoro-6-(trifluoromethyl) quinazolin-4-yl)-3-oxa-7,9-diazabicyclo[3.3.1]nonane-7-carboxylate

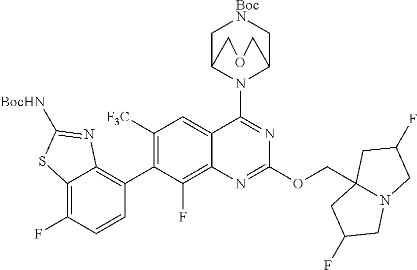 [SMILES: CC(C)(C)OC(=O)Nc1nc2c(-c3c(C(F)(F)F)cc4c(N5C6COCC5CN(C(=O)OC(C)(C)C)C6)nc(OCC56CC(F)CN5CC(F)C6)nc4c3F)ccc(F)c2s1]

To a solution of tert-butyl 9-(7-bromo-2-((2,6-difluorotetrahydro-1H-pyrrolizin-7a(5H)-yl)methoxy)-8-fluoro-6-(trifluoromethyl)quinazolin-4-yl)-3-oxa-7,9-diazabicyclo[3.3.1] nonane-7-carboxylate (69.6 mg, 0.1 mmol), (2-((tert-butoxycarbonyl)amino)-7-fluorobenzo[d] thiazol-4-yl)boronic acid (94 mg, 0.3 mmol), Antphos (7.4 mg, 0.02 mmol), Pd₂(dba)₃ (9 mg, 0.01 mmol) and K₃PO₄ (64 mg, 0.3 mmol) in dioxane/H₂O (5 mL/1 mL) was stirred at 100° C. for 5 hrs. After completion, the reaction mixture was purified by Prep-TLC (PE:EA=1:3) to give the title product (80 mg, crude). MS (ESI, m/e) [M+1]⁺884.6.

### Step 3: 4-(4-(3-oxa-7,9-diazabicyclo[3.3.1]nonan-9-yl)-2-((2,6-difluorotetrahydro-1H-pyrrolizin-7a(5H)-yl)methoxy)-8-fluoro-6-(trifluoromethyl)quinazolin-7-yl)-7-fluorobenzo [d]thiazol-2-amine

To a solution of tert-butyl 9-(7-(2-((tert-butoxycarbonyl)amino)-7-fluorobenzo[d]thiazol-4-yl)-2-((2,6-difluorotetrahydro-1H-pyrrolizin-7a(5H)-yl)methoxy)-8-fluoro-6-(trifluoro methyl)quinazolin-4-yl)-3-oxa-7,9-diazabicyclo[3.3.1]nonane-7-carboxylate (80 mg, 0.09 mmol) in DCM (4 mL) was added TFA (2 mL) at room temperature. The resulting mixture was stirred at room temperature for 3 hrs. After completion, the reaction mixture was concentrated and the residue was purified by prep-HPLC to give the title product (33.4 mg, 40.0%). ¹H NMR (500 MHz, CD₃OD) δ 8.03 (s, 1H), 7.20-7.17 (m, 1H), 7.00-6.96 (m, 1H), 5.52-5.37 (m, 2H), 4.80-4.75 (m, 2H), 4.57 (s, 2H), 4.35-4.20 (m, 4H), 3.87-3.69 (m, 5H), 3.65-3.39 (m, 3H), 2.63-2.38 (m, 4H). MS (ESI, m/e) [M+1]⁺683.8.

### Example 140: 4-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-6-chlorobenzo [d]thiazol-2-amine

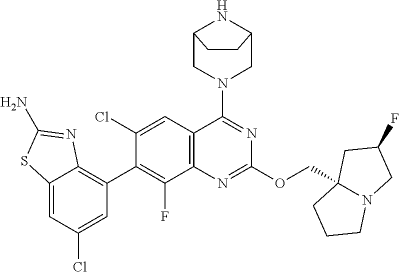 [SMILES: Nc1nc2c(-c3c(Cl)cc4c(N5CC6CCC(C5)N6)nc(OC[C@@]56CCCN5C[C@H](F)C6)nc4c3F)cc(Cl)cc2s1]

Synthetic Route

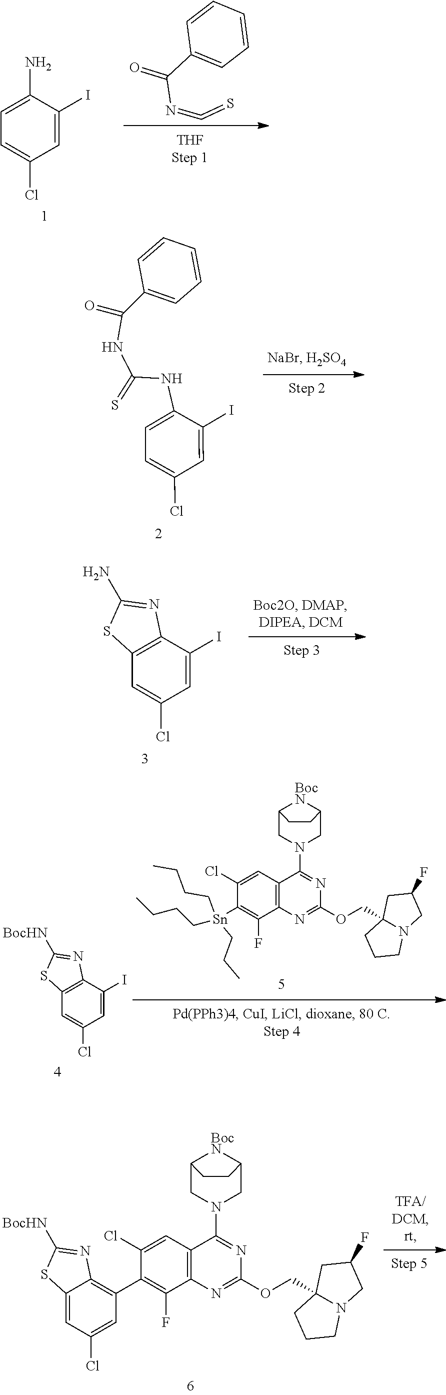

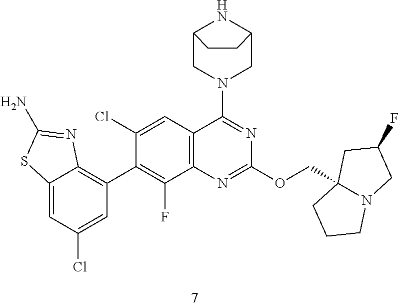 [SMILES: Nc1nc2c(-c3c(Cl)cc4c(N5CC6CCC(C5)N6)nc(OC[C@@]56CCCN5C[C@H](F)C6)nc4c3F)cc(Cl)cc2s1]

### Step 1: N-((4-chloro-2-iodophenyl)carbamothioyl)benzamide

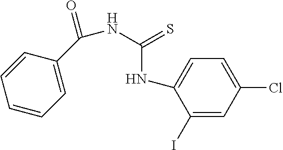 [SMILES: O=C(NC(=S)Nc1ccc(Cl)cc1I)c1ccccc1]

To a solution of 4-chloro-2-iodoaniline (7.58 g, 30.0 mmol) in THF (150 mL) was added benzoyl isothiocyanate (4.89 g, 30.0 mmol) at room temperature. The resulting mixture was stirred at room temperature for 16 hrs. After completion, the reaction mixture was concentrated to give the crude product (10 g). MS (ESI, m/e) [M+H]⁺ 416.9.

### Step 2: 6-chloro-4-iodobenzo[d]thiazol-2-amine

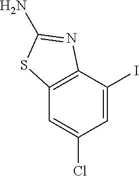 [SMILES: Nc1nc2c(I)cc(Cl)cc2s1]

To a solution of N-((4-chloro-2-iodophenyl)carbamothioyl)benzamide (4.16 g) in H₂SO₄ (50 mL) was added NaBr (206 mg) at room temperature. The resulting mixture was stirred at 85° C. for 16 hrs. After completion, the reaction mixture was poured into ice water, neutralized with NaOH solution, and filtered to give the title product (3 g). MS (ESI, m/e) [M+H]⁺ 310.9.

### Step 3: tert-butyl (6-chloro-4-iodobenzo[d]thiazol-2-yl)carbamate

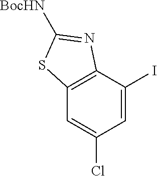 [SMILES: CC(C)(C)OC(=O)Nc1nc2c(I)cc(Cl)cc2s1]

To a solution of 6-chloro-4-iodobenzo[d]thiazol-2-amine (1.55 g) in DCM (25 mL) was added di-tert-butyl dicarbonate (1.08 g), DMAP (30.5 mg) and DIPEA (1.2 g) at room temperature. The resulting mixture was stirred at room temperature for 3 hrs. After completion, the reaction mixture was directly concentrated to give the residue. The residue was purified by silica gel column chromatography, eluted with 0-30% EA in PE to give the title product (1.0 g). MS (ESI, m/e) [M+H]⁺ 410.9.

### Step 4: tert-butyl 3-(7-(2-((tert-butoxycarbonyl)amino)-6-chlorobenzo[d]thiazol-4-yl)-6-chloro-8-fluoro-2-(((2R,7aS)-2-fluorotetrahydro-1H-pyrrolizin-7a(5H)-yl)methoxy) quinazolin-4-yl)-3,8-diazabicyclo[3.2.1]octane-8-carboxylate

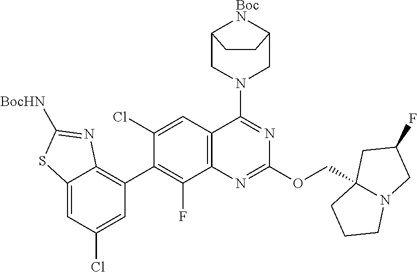 [SMILES: CC(C)(C)OC(=O)Nc1nc2c(-c3c(Cl)cc4c(N5CC6CCC(C5)N6C(=O)OC(C)(C)C)nc(OC[C@@]56CCCN5C[C@H](F)C6)nc4c3F)cc(Cl)cc2s1]

A solution of tert-butyl 3-(6-chloro-7-(dibutyl(propyl)stannyl)-8-fluoro-2-(((2R,7aS)-2-fluorotetrahydro-1H-pyrrolizin-7a(5H)-yl)methoxy)quinazolin-4-yl)-3,8-diaza bicyclo[3.2.1]octane-8-carboxylate (314 mg, 0.375 mmol), tert-butyl (6-chloro-4-iodobenzo [d]thiazol-2-yl)carbamate (103 mg, 0.25 mmol), Pd(PPh₃)₄ (144 mg, 0.125 mmol), CuI (29 mg, 0.15 mmol) and LiCl (32 mg, 0.75 mmol) in dioxane (10 mL) was stirred at 100° C. for 5 hrs. After completion, the reaction mixture was purified by Prep-TLC (PE:EA=1:2) to give the title product (30 mg, 9.9%). MS (ESI, m/e) [M+H]⁺ 832.

### Step 5: 4-(4-(3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-6-chlorobenzo[d]thiazol-2-amine

To a solution of tert-butyl 3-(7-(2-((tert-butoxycarbonyl)amino)-6-chlorobenzo[d]thiazol-4-yl)-6-chloro-8-fluoro-2-(((2R,7aS)-2-fluorotetrahydro-1H-pyrrolizin-7a(5H)-yl) methoxy)quinazolin-4-yl)-3,8-diazabicyclo[3.2.1]octane-8-carboxylate (30 mg, 0.0361 mmol) in DCM (5 mL) was added TFA (1 mL) at room temperature. The resulting mixture was stirred at room temperature for 4 hrs. After completion, the reaction mixture was concentrated and the residue was purified by prep-HPLC to give the title product (2.67 mg, FA salt, 10.9%). ¹H NMR (500 MHz, CD₃OD) δ 8.46 (s, 1H), 7.91 (s, 1H), 7.77 (d, J=2.1 Hz, 1H), 7.20 (d, J=2.1 Hz, 1H), 5.47-5.36 (m, 1H), 4.66-4.34 (m, 4H), 3.88 (s, 2H), 3.73-3.68 (m, 2H), 3.53-3.44 (m, 3H), 3.28-3.16 (m, 1H), 2.59-1.83 (m, 10H). MS (ESI, m/e) [M+H]⁺ 631.9.

### Example 141: 3-((7-(2-amino-7-fluorobenzo[d]thiazol-4-yl)-4-(3,8-diazabicyclo [3.2.1]octan-3-yl)-8-fluoro-6-(trifluoromethyl)quinazolin-2-yl)oxy)propane-1,2-diol

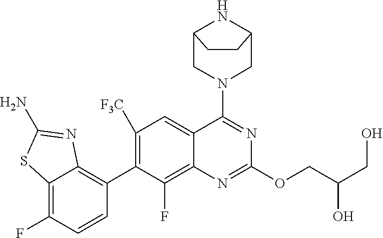 [SMILES: Nc1nc2c(-c3c(C(F)(F)F)cc4c(N5CC6CCC(C5)N6)nc(OCC(O)CO)nc4c3F)ccc(F)c2s1]

Example 141 was prepared by similar procedure as described in Example 145 by replacing (tetrahydrofuran-3-yl)methanol with (2,2-dimethyl-1,3-dioxolan-4-yl)methanol to give the title product (2.46 mg). 1H NMR (500 MHz, DMSO-d₆) δ 8.07 (s, 1H), 7.93 (s, 2H), 7.19-7.18 (m, 1H), 7.06-7.02 (m, 1H), 5.03-5.02 (m, 1H), 4.74-4.73 (m, 1H), 4.59-4.56 (m, 1H), 4.43-4.41 (m, 2H), 4.27-4.17 (m, 3H), 3.85-3.73 (m, 4H), 3.43-3.38 (m, 2H), 1.94-1.92 (m, 4H). MS (ESI, m/e) [M+H]⁺ 583.10.

### Example 142: 3-((7-(2-amino-7-fluorobenzo[d]thiazol-4-yl)-4-(3,8-diazabicyclo [3.2.1]octan-3-yl)-8-fluoro-6-(trifluoromethyl)quinazolin-2-yl)oxy)-2-methylpropan-1-ol

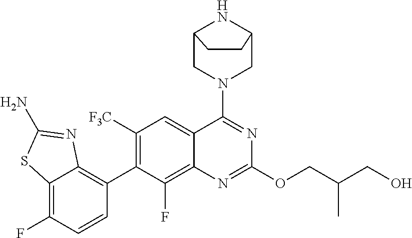 [SMILES: CC(CO)COc1nc(N2CC3CCC(C2)N3)c2cc(C(F)(F)F)c(-c3ccc(F)c4sc(N)nc34)c(F)c2n1]

Example 142 was prepared by similar procedure as described in Example 145 by replacing (tetrahydrofuran-3-yl)methanol with 2-methyl propane-1,3-diol to give the title product (5 mg, FA salt, 26% yield). 1H NMR (400 MHz, CD₃OD) δ 8.46 (s, 1H), 8.11 (s, 1H), 7.20-7.17 (m, 1H), 6.99-6.95 (m, 1H), 4.68-4.65 (m, 2H), 4.53-4.42 (m, 1H), 4.42-4.30 (m, 1H), 4.06 (s, 2H), 3.85-3.80 (m, 2H), 3.64-3.53 (m, 2H), 2.18-2.10 (m, 1H), 2.07-2.00 (m, 4H), 1.07 (d, J=6.9 Hz, 3H). MS (ESI, m/e) [M+H]⁺ 581.4.

### Example 143: 4-(4-(3,8-diazabicyclo[3.2.1]octan-3-yl)-8-fluoro-2-((tetrahydrofuran-3-yl)oxy-6-(trifluoromethyl)quinazolin-7-yl)-7-fluorobenzo[d]thiazol-2-amine

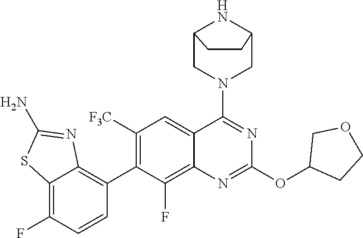 [SMILES: Nc1nc2c(-c3c(C(F)(F)F)cc4c(N5CC6CCC(C5)N6)nc(OC5CCOC5)nc4c3F)ccc(F)c2s1]

Example 143 was prepared by similar procedure as described in Example 145 by replacing (tetrahydrofuran-3-yl)methanol with tetrahydrofuran-3-ol to give the title product (4 mg, 12% yield). ¹H NMR (400 MHz, CD₃OD) δ 8.12 (s, 1H), 7.20-7.17 (m, 1H), 6.98-6.94 (m, 1H), 5.69-5.67 (m, 1H), 4.60-4.53 (m, 2H), 4.09-3.72 (m, 8H), 2.33-2.19 (m, 2H), 1.95-1.89 (m, 4H). MS (ESI, m/e) [M+H]⁺ 579.5.

### Example 144: 4-(4-(3,8-diazabicyclo[3.2.1]octan-3-yl)-8-fluoro-2-((tetrahydro-2H-pyran-4-yl)methoxy)-6-(trifluoromethyl)quinazolin-7-yl)-7-fluorobenzo[d]thiazol-2-amine

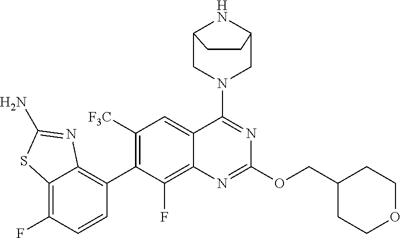 [SMILES: Nc1nc2c(-c3c(C(F)(F)F)cc4c(N5CC6CCC(C5)N6)nc(OCC5CCOCC5)nc4c3F)ccc(F)c2s1]

Example 144 was prepared by similar procedure as described in Example 145 by replacing (tetrahydrofuran-3-yl)methanol with (tetrahydro-2H-pyran-4-yl)methanol to give the title product (8 mg, FA salt, 16% yield). 1H NMR (400 MHz, CD₃OD) δ 8.48 (s, 1H), 8.11 (s, 1H), 7.20-7.17 (m, 1H), 6.98-6.95 (m, 1H), 4.58-5.54 (m, 2H), 4.34-4.30 (m, 2H), 4.10-3.90 (m, 4H), 3.80-3.70 (m, 2H), 3.46-3.44 (m, 2H), 2.18-2.10 (m, 1H), 2.04-1.95 (m, 4H), 1.79-1.77 (m, 2H), 1.49-1.45 (m, 2H). MS (ESI, m/e) [M+H]⁺ 607.4.

### Example 145: 4-(4-(3,8-diazabicyclo[3.2.1]octan-3-yl)-8-fluoro-2-((tetrahydrofuran-3-yl)methoxy)-6-(trifluoromethyl)quinazolin-7-yl)-7-fluorobenzo [d]thiazol-2-amine

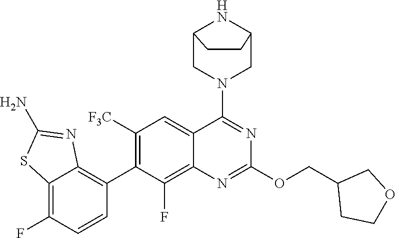 [SMILES: Nc1nc2c(-c3c(C(F)(F)F)cc4c(N5CC6CCC(C5)N6)nc(OCC5CCOC5)nc4c3F)ccc(F)c2s1]

Synthetic Route

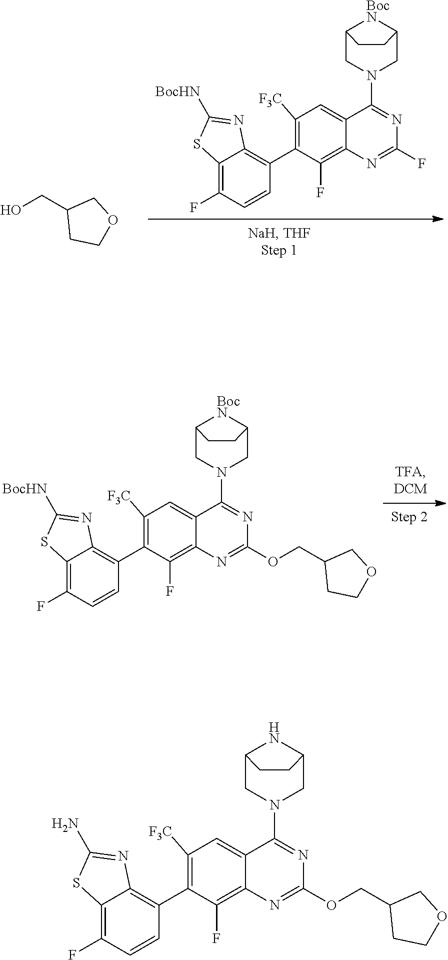 [SMILES: OCC1CCOC1 ; CC(C)(C)OC(=O)Nc1nc2c(-c3c(C(F)(F)F)cc4c(N5CC6CCC(C5)N6C(=O)OC(C)(C)C)nc(F)nc4c3F)ccc(F)c2s1 ; CC(C)(C)OC(=O)Nc1nc2c(-c3c(C(F)(F)F)cc4c(N5CC6CCC(C5)N6C(=O)OC(C)(C)C)nc(OCC5CCOC5)nc4c3F)ccc(F)c2s1 ; Nc1nc2c(-c3c(C(F)(F)F)cc4c(N5CC6CCC(C5)N6)nc(OCC5CCOC5)nc4c3F)ccc(F)c2s1]

### Step 1: tert-butyl 3-(7-(2-((tert-butoxycarbonyl)amino)-7-fluorobenzo[d]thiazol-4-yl)-8-fluoro-2-((tetrahydrofuran-3-yl)methoxy)-6-(trifluoromethyl)quinazolin-4-yl)-3,8-diaza bicyclo[3.2.1]octane-8-carboxylate

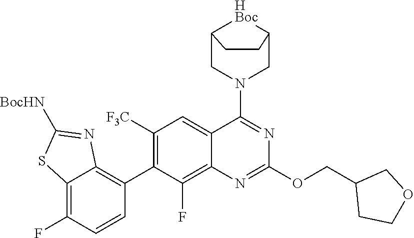

To a solution of (tetrahydrofuran-3-yl)methanol (30.6 mg, 0.30 mmol) in THF (5 mL) was added sodium hydride (13.42 mg, 0.33 mmol) at room temperature. The resulting mixture was stirred at room temperature for 1 hrs. Then, tert-butyl 3-(7-(2-((tert-butoxy carbonyl)amino)-7-fluorobenzo[d]thiazol-4-yl)-2,8-difluoro-6-(trifluoromethyl)quinazolin-4-yl)-3,8-diazabicyclo[3.2.1]octane-8-carboxylate (71.0 mg, 0.10 mmol) was added to the reaction mixture and stirred at room temperature for 1 hrs. After completion, the reaction mixture was diluted with EA (50 mL) and washed with brine (15 mL×3). The organic layer was dried over anhydrous Na₂SO₄, filtered and the filtrate was concentrated to give the residue. The residue was purified by Prep-TLC (PE:EA=3:1) to give the title product (40 mg, 0.05 mmol). MS (ESI, m/e) [M+H]⁺ 793.3.

### Step 2: 4-(4-(3,8-diazabicyclo[3.2.1]octan-3-yl)-8-fluoro-2-((tetrahydrofuran-3-yl)methoxy)-6-(trifluoromethyl)quinazolin-7-yl)-7-fluorobenzo[d]thiazol-2-amine

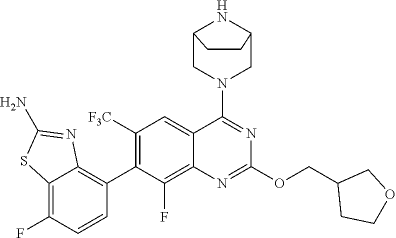 [SMILES: Nc1nc2c(-c3c(C(F)(F)F)cc4c(N5CC6CCC(C5)N6)nc(OCC5CCOC5)nc4c3F)ccc(F)c2s1]

To a solution of tert-butyl 3-(7-(2-((tert-butoxycarbonyl)amino)-7-fluorobenzo[d]thiazol-4-yl)-8-fluoro-2-((tetrahydrofuran-3-yl)methoxy)-6-(trifluoromethyl)quinazolin-4-yl)-3,8-diazabicyclo[3.2.1]octane-8-carboxylate (40 mg, 0.05 mmol) in DCM (5 mL) was added TFA (5 mL) at room temperature. The resulting mixture was stirred at room temperature for 3 hrs. After completion, the reaction mixture was concentrated to give the residue. The residue was purified by pre-HPLC to give the title product (6.29 mg). ¹H NMR (500 MHz, DMSO-d₆) δ 8.07 (s, 1H), 7.90 (s, 2H), 7.19-7.17 (m, 1H), 7.05-7.02 (m, 1H), 4.41-4.24 (m, 4H), 3.79-3.75 (m, 4H), 3.65-3.52 (m, 5H), 2.71-2.66 (m, 1H), 2.03-1.96 (m, 1H), 1.72-1.67 (m, 5H). MS (ESI, m/e) [M+H]⁺ 593.6.

### Example 146: 4-(4-(3,8-diazabicyclo[3.2.1]octan-3-yl)-8-fluoro-2-((tetrahydro-2H-pyran-2-yl)methoxy)-6-(trifluoromethyl)quinazolin-7-yl)-7-fluorobenzo[d]thiazol-2-amine

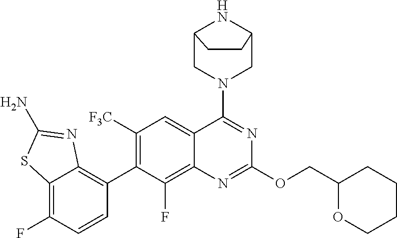 [SMILES: Nc1nc2c(-c3c(C(F)(F)F)cc4c(N5CC6CCC(C5)N6)nc(OCC5CCCCO5)nc4c3F)ccc(F)c2s1]

Example 146 was prepared by similar procedure as described in Example 145 by replacing (tetrahydrofuran-3-yl)methanol with (tetrahydro-2H-pyran-2-yl)methanol to give the title product (16.8 mg). ¹H NMR (500 MHz, DMSO-d₆) δ 8.07 (s, 1H), 7.91 (s, 2H), 7.20-7.17 (m, 1H), 7.05-7.02 (m, 1H), 4.41-4.38 (m, 1H), 4.30-4.27 (m, 3H), 3.89-3.87 (m, 1H), 3.66-3.64 (m, 4H), 3.27-3.55 (m, 1H), 3.40-3.36 (m, 2H), 1.81-1.79 (m, 1H), 1.68-1.61 (m, 5H), 1.52-1.45 (m, 3H), 1.36-1.30 (m, 1H). MS (ESI, m/e) [M+H]⁺ 607.4.

### Example 147: 1-(((7-(2-amino-7-fluorobenzo[d]thiazol-4-yl)-4-(3,8-diazabicyclo [3.2.1]octan-3-yl)-8-fluoro-6-(trifluoromethyl)quinazolin-2-yl)oxy)methyl)cyclopropane-1-carbonitrile

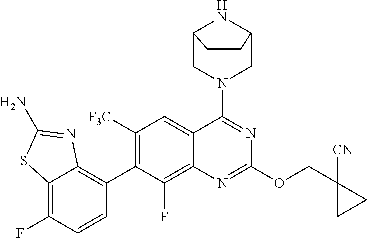 [SMILES: N#CC1(COc2nc(N3CC4CCC(C3)N4)c3cc(C(F)(F)F)c(-c4ccc(F)c5sc(N)nc45)c(F)c3n2)CC1]

Example 147 was prepared by similar procedure as described in Example 145 by replacing (tetrahydrofuran-3-yl)methanol with 1-(hydroxymethyl)cyclopropane-1-carbonitrile to give the title product (7.36 mg, FA salt). 1H NMR (500 MHz, DMSO-d₆) δ 8.22 (s, 1H), 8.10 (s, 1H), 7.90 (s, 2H), 7.21-7.18 (m, 1H), 7.06-7.02 (m, 1H), 4.43-4.30 (m, 5H), 3.66-3.54 (m, 4H), 1.68-1.64 (m, 4H), 1.39-1.35 (m, 2H), 1.25-1.24 (m, 2H). MS (ESI, m/e) [M+H]⁺ 588.4.

### Example 148: 3-((7-(2-amino-7-fluorobenzo[d]thiazol-4-yl)-4-(3,8-diazabicyclo [3.2.1]octan-3-yl)-8-fluoro-6-(trifluoromethyl)quinazolin-2-yl)oxy)thietane 1,1-dioxide

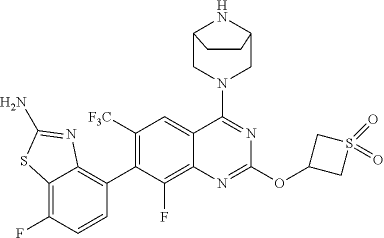 [SMILES: Nc1nc2c(-c3c(C(F)(F)F)cc4c(N5CC6CCC(C5)N6)nc(OC5CS(=O)(=O)C5)nc4c3F)ccc(F)c2s1]

Example 148 was prepared by similar procedure as described in Example 145 by replacing (tetrahydrofuran-3-yl)methanol with 3-hydroxy thietane 1,1-dioxide to give the title product (2 mg, 11% yield). 1H NMR (400 MHz, CD₃OD) δ 8.14 (s, 1H), 7.20-7.17 (m, 1H), 6.99-6.95 (m, 1H), 5.66-5.63 (m, 1H), 4.79-4.76 (m, 2H), 4.67-4.63 (m, 2H), 4.36-4.30 (m, 2H), 4.10-4.06 (m, 2H), 3.86-3.82 (m, 2H), 2.10-2.00 (m, 4H). MS (ESI, m/e) [M+H]⁺ 613.4.

### Example 149: (1-(((7-(2-amino-7-fluorobenzo[d]thiazol-4-yl)-4-(3,8-diaza bicyclo[3.2.1]octan-3-yl)-8-fluoro-6-(trifluoromethyl)quinazolin-2-yl)oxy)methyl) cyclopropyl) methanol

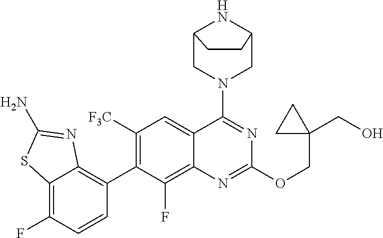 [SMILES: Nc1nc2c(-c3c(C(F)(F)F)cc4c(N5CC6CCC(C5)N6)nc(OCC5(CO)CC5)nc4c3F)ccc(F)c2s1]

Example 149 was prepared by similar procedure as described in Example 145 by replacing (tetrahydrofuran-3-yl)methanol with cyclopropane-1,1-diyldimethanol to give the title product (3 mg, FA salt, 13% yield). ¹H NMR (400 MHz, CD₃OD) δ 8.48 (s, 1H), 8.11 (s, 1H), 7.20-7.17 (m, 1H), 6.98-6.95 (m, 1H), 4.64-6.57 (m, 2H), 4.45-4.40 (m, 2H), 3.97-3.89 (m, 2H), 3.80-3.75 (m, 2H), 3.44 (s, 2H), 2.04-1.93 (m, 4H), 0.66-0.60 (m, 4H). MS (ESI, m/e) [M+H]⁺ 593.4.

### Example 150: 4-(4-(3,8-diazabicyclo[3.2.1]octan-3-yl)-8-fluoro-2-((3-methoxy cyclobutyl)methoxy)-6-(trifluoromethyl)quinazolin-7-yl)-7-fluorobenzo[d]thiazol-2-amine

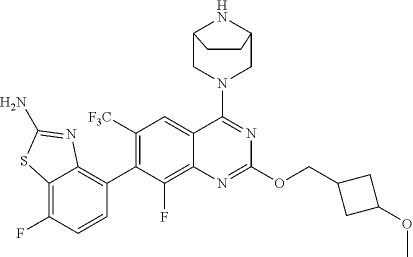 [SMILES: COC1CC(COc2nc(N3CC4CCC(C3)N4)c3cc(C(F)(F)F)c(-c4ccc(F)c5sc(N)nc45)c(F)c3n2)C1]

Example 150 was prepared by similar procedure as described in Example 145 by replacing (tetrahydrofuran-3-yl)methanol with (3-methoxycyclobutyl)methanol to give the title product (4.68 mg, FA salt)¹H NMR (500 MHz, CD₃OD) δ 8.49 (s, 1H), 8.11 (s, 1H), 7.24-7.15 (m, 1H), 6.98-6.94 (m, 1H), 4.66-4.53 (m, 2H), 4.48-4.39 (m, 2H), 3.98-3.81 (m, 3H), 3.78-3.73 (m, 2H), 3.23 (s, 3H), 2.50-2.35 (m, 2H), 2.23-2.15 (m, 1H), 2.05-1.88 (m, 4H), 1.82-1.77 (m, 2H). MS (ESI, m/e) [M+H]⁺ 607.4.

### Example 151: 3-((7-(2-amino-7-fluorobenzo[d]thiazol-4-yl)-4-(3,8-diaza bicyclo[3.2.1]octan-3-yl)-8-fluoro-6-(trifluoromethyl)quinazolin-2-yl)oxy)-1-fluorocyclo butane-1-carbonitrile

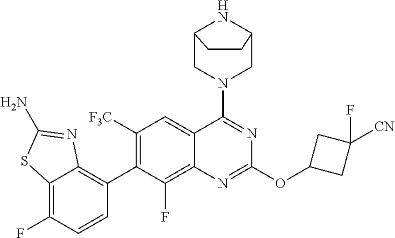 [SMILES: N#CC1(F)CC(Oc2nc(N3CC4CCC(C3)N4)c3cc(C(F)(F)F)c(-c4ccc(F)c5sc(N)nc45)c(F)c3n2)C1]

Example 151 was prepared by similar procedure as described in Example 145 by replacing (tetrahydrofuran-3-yl)methanol with 1-fluoro-3-hydroxycyclobutane-1-carbonitrile to give the title product (1.17 mg). ¹H NMR (500 MHz, CD₃OD) δ 8.13 (s, 1H), 7.22-7.12 (m, 1H), 6.99-6.95 (m, 1H), 5.57-5.51 (m, 1H), 4.67-4.63 (m, 2H), 4.13-4.09 (m, 2H), 3.86-3.83 (m, 2H), 3.34-3.20 (m, 3H), 3.08-3.01 (m, 1H), 2.10-2.02 (m, 4H). MS (ESI, m/e) [M+H]⁺ 606.4.

### Example 152: 4-(2-((2-oxabicyclo[2.1.1]hexan-4-yl)methoxy)-4-(3,8-diaza bicyclo[3.2.1]octan-3-yl)-8-fluoro-6-(trifluoromethyl)quinazolin-7-yl)-7-fluorobenzo[d] thiazol-2-amine

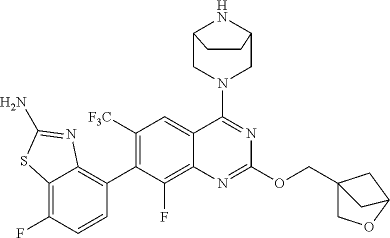 [SMILES: Nc1nc2c(-c3c(C(F)(F)F)cc4c(N5CC6CCC(C5)N6)nc(OCC56COC(C5)C6)nc4c3F)ccc(F)c2s1]

Example 152 was prepared by similar procedure as described in Example 145 by replacing (tetrahydrofuran-3-yl)methanol with (2-oxabicyclo[2.1.1]hexan-4-yl)methanol to give the title product (6.63 mg). ¹H NMR (500 MHz, CD₃OD) δ 8.47 (s, 1H), 8.12 (s, 1H), 7.26-7.15 (m, 1H), 6.98-6.95 (m, 1H), 4.78-4.75 (m, 2H), 4.65-4.55 (m, 3H), 3.94 (s, 2H), 3.81-3.71 (m, 4H), 2.04-1.93 (m, 6H), 1.64-1.60 (m, 2H). MS (ESI, m/e) [M+H]⁺ 605.4.

### Example 153: 3-((7-(2-amino-7-fluorobenzo[d]thiazol-4-yl)-4-(3,8-diazabicyclo [3.2.1]octan-3-yl)-8-fluoro-6-(trifluoromethyl)quinazolin-2-yl)oxy)-2,2-difluoropropan-1-ol

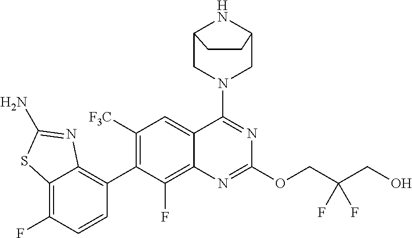 [SMILES: Nc1nc2c(-c3c(C(F)(F)F)cc4c(N5CC6CCC(C5)N6)nc(OCC(F)(F)CO)nc4c3F)ccc(F)c2s1]

Example 153 was prepared by similar procedure as described in Example 145 by replacing (tetrahydrofuran-3-yl)methanol with 2,2-difluoropropane-1,3-diol to give the title product (5.5 mg, FA salt). 1H NMR (500 MHz, DMSO-d₆) δ 8.17 (s, 1H), 8.11 (s, 1H), 7.91 (s, 2H), 7.21-7.18 (m, 1H), 7.06-7.02 (m, 1H), 5.69-5.67 (m, 1H), 4.72-4.67 (m, 2H), 4.43-4.28 (m, 2H), 3.78-3.52 (m, 7H), 1.65-1.63 (m, 4H). MS (ESI, m/e) [M+H]⁺ 603.4.

### Example 154: (1-(((7-(2-amino-7-fluorobenzo[d]thiazol-4-yl)-4-(3,8-diaza bicyclo[3.2.1]octan-3-yl)-8-fluoro-6-(trifluoromethyl)quinazolin-2-yl)oxy)methyl)cyclobutyl) methanol

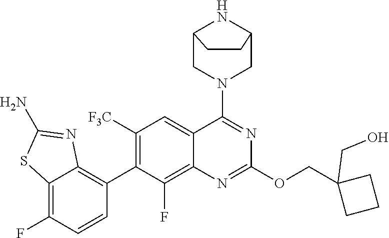 [SMILES: Nc1nc2c(-c3c(C(F)(F)F)cc4c(N5CC6CCC(C5)N6)nc(OCC5(CO)CCC5)nc4c3F)ccc(F)c2s1]

Example 154 was prepared by similar procedure as described in Example 145 by replacing (tetrahydrofuran-3-yl)methanol with cyclobutane-1,1-diyldimethanol to give the title product (14.23 mg, FA salt). 1H NMR (500 MHz, DMSO-d₆) δ 8.22 (s, 1H), 8.07 (s, 1H), 7.91 (s, 2H), 7.21-7.18 (m, 1H), 7.06-7.02 (m, 1H), 4.40-4.25 (m, 5H), 3.64-3.48 (m, 6H), 1.89-1.84 (m, 6H), 1.67-1.63 (m, 4H). MS (ESI, m/e) [M+H]⁺ 607.4.

### Example 155: 1-(2-((7-(2-amino-7-fluorobenzo[d]thiazol-4-yl)-4-(3,8-diaza bicyclo[3.2.1]octan-3-yl)-6-chloro-8-fluoroquinazolin-2-yl)oxy)ethyl)cyclopropane-1-carbonitrile

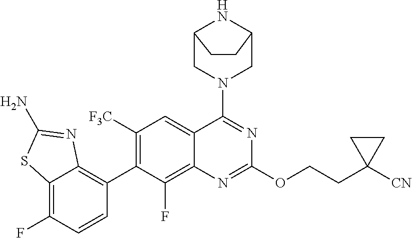 [SMILES: N#CC1(CCOc2nc(N3CC4CCC(C3)N4)c3cc(C(F)(F)F)c(-c4ccc(F)c5sc(N)nc45)c(F)c3n2)CC1]

Example 155 was prepared by similar procedure as described in Example 145 by replacing (tetrahydrofuran-3-yl)methanol with 1-(2-hydroxyethyl)cyclopropane-1-carbonitrile to give the title product (12.98 mg, 27% yield for 2 steps). ¹H NMR (500 MHz, CD₃OD) δ 8.13 (s, 1H), 7.20 (dd, J=8.1, 5.5 Hz, 1H), 6.97 (t, J=8.8 Hz, 1H), 4.75-4.65 (m, 4H), 4.11 (d, J=14.4 Hz, 2H), 3.87 (dd, J=13.7, 6.1 Hz, 2H), 2.18-1.96 (m, 6H), 1.33-1.18 (m, 2H), 1.11-0.97 (m, 2H). MS (ESI, m/e) [M+H]⁺ 602.4.

### Example 156: 4-(4-(3,8-diazabicyclo[3.2.1]octan-3-yl)-6-chloro-8-fluoro-2-((1-methoxycyclopropyl)methoxy)quinazolin-7-yl)-7-fluorobenzo[d]thiazol-2-amine

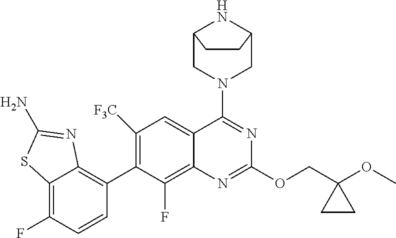 [SMILES: COC1(COc2nc(N3CC4CCC(C3)N4)c3cc(C(F)(F)F)c(-c4ccc(F)c5sc(N)nc45)c(F)c3n2)CC1]

Example 156 was prepared by similar procedure as described in Example 145 by replacing (tetrahydrofuran-3-yl)methanol with (1-methoxycyclopropyl)methanol to give the title product (6.24 mg, 14% yield for 2 steps). H NMR (500 MHz, CD₃OD) δ 8.12 (s, 1H), 7.27-7.14 (m, 1H), 6.97 (t, J=8.8 Hz, 1H), 4.73-4.44 (m, 4H), 3.91 (s, 2H), 3.77 (t, J=13.7 Hz, 2H), 3.42 (s, 3H), 1.97 (s, 4H), 0.91 (d, J=5.3 Hz, 2H), 0.80 (d, J=5.1 Hz, 2H). MS (ESI, m/e) [M+H]⁺ 593.4.

### Example 157: 1-(((7-(2-amino-7-fluorobenzo[d]thiazol-4-yl)-4-(3,8-diazabicyclo [3.2.1]octan-3-yl)-8-fluoro-6-(trifluoromethyl)quinazolin-2-yl)oxy)methyl)cyclohexane-1-carbonitrile

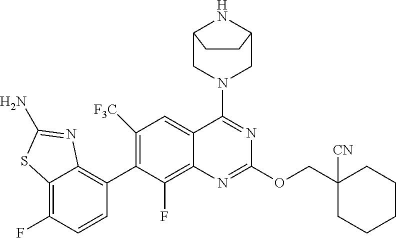 [SMILES: N#CC1(COc2nc(N3CC4CCC(C3)N4)c3cc(C(F)(F)F)c(-c4ccc(F)c5sc(N)nc45)c(F)c3n2)CCCCC1]

Example 157 was prepared by similar procedure as described in Example 145 by replacing (tetrahydrofuran-3-yl)methanol with 1-(hydroxymethyl)cyclohexane-1-carbonitrile to give the title product (4 mg, 16% yield). 1H NMR (400 MHz, CD₃OD) δ 8.44 (s, 1H), 8.14 (s, 1H), 7.21-7.18 (m, 1H), 6.99-6.95 (m, 1H), 4.69-4.65 (m, 2H), 4.57-4.53 (m, 2H), 4.05-3.99 (m, 4H), 3.85-3.82 (m, 2H), 3.76-3.69 (m, 2H), 2.05-1.99 (m, 6H), 1.88-1.82 (m, 2H). MS (ESI, m/e) [M+H]⁺ 632.4.

### Example 158: (1-(((7-(2-amino-7-fluorobenzo[d]thiazol-4-yl)-4-(3,8-diaza bicyclo[3.2.1]octan-3-yl)-8-fluoro-6-(trifluoromethyl)quinazolin-2-yl)oxy)methyl)-3-fluoro cyclobutyl)methanol

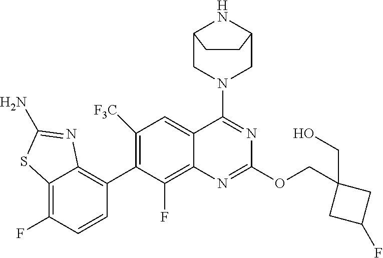 [SMILES: Nc1nc2c(-c3c(C(F)(F)F)cc4c(N5CC6CCC(C5)N6)nc(OCC5(CO)CC(F)C5)nc4c3F)ccc(F)c2s1]

Example 158 was prepared by similar procedure as described in Example 145 by replacing (tetrahydrofuran-3-yl)methanol with (3-fluorocyclobutane-1,1-diyl)dimethanol to give the title product. (2 mg, FA salt, 12% yield). 1H NMR (400 MHz, CD₃OD) δ 8.46 (s, 1H), 8.12 (s, 1H), 7.21-7.18 (m, 1H), 6.98-6.95 (m, 1H), 5.24-5.00 (m, 1H), 4.66-4.61 (m, 2H), 4.51-4.44 (m, 2H), 4.03-3.96 (m, 2H), 3.66-3.62 (m, 2H), 2.44-2.39 (m, 2H), 2.21-2.13 (m, 2H), 2.05-1.95 (m, 4H). MS (ESI, m/e) [M+H]⁺ 625.5.

### Example 159: 4-(4-(3,8-diazabicyclo[3.2.1]octan-3-yl)-8-fluoro-2-((3-fluorotetra hydro-2H-pyran-4-yl)oxy)-6-(trifluoromethyl)quinazolin-7-yl)-7-fluorobenzo[d]thiazol-2-amine

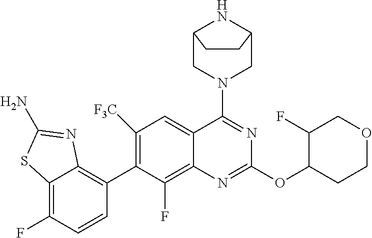 [SMILES: Nc1nc2c(-c3c(C(F)(F)F)cc4c(N5CC6CCC(C5)N6)nc(OC5CCOCC5F)nc4c3F)ccc(F)c2s1]

Example 159 was prepared by similar procedure as described in Example 145 by replacing (tetrahydrofuran-3-yl)methanol with 3-fluoro tetrahydro-2H-pyran-4-ol to give the title product (2 mg, FA salt, 12% yield). ¹H NMR (400 MHz, CD₃OD) δ 8.49 (s, 1H), 8.12 (s, 1H), 7.20-7.17 (m, 1H), 6.98-6.95 (m, 1H), 5.49-5.42 (m, 1H), 5.00-4.85 (m, 1H), 4.56-4.49 (m, 2H), 4.07-3.94 (m, 4H), 3.78-3.62 (m, 4H), 2.25-2.17 (m, 1H), 2.05-1.95 (m, 5H). MS (ESI, m/e) [M+H]⁺ 611.5.

### Example 160: 4-((7-(2-amino-7-fluorobenzo[d]thiazol-4-yl)-4-(3,8-diaza bicyclo[3.2.1]octan-3-yl)-6-chloro-8-fluoroquinazolin-2-yl)oxy)tetrahydro-2H-thiopyran 1,1-dioxide

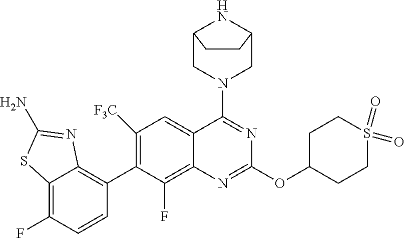 [SMILES: Nc1nc2c(-c3c(C(F)(F)F)cc4c(N5CC6CCC(C5)N6)nc(OC5CCS(=O)(=O)CC5)nc4c3F)ccc(F)c2s1]

Example 160 was prepared by similar procedure as described in Example 145 by replacing (tetrahydrofuran-3-yl)methanol with 4-hydroxytetrahydro-2H-thiopyran 1,1-dioxide to give the title product (16.49 mg, FA salt, 36% yield for 2 steps). ¹H NMR (500 MHz, CD₃OD) δ 8.49 (s, 1H), 8.14 (s, 1H), 7.21 (ddd, J=14.5, 8.0, 5.6 Hz, 1H), 6.97 (t, J=8.8 Hz, 1H), 5.66-5.44 (m, 1H), 4.64 (t, J=12.0 Hz, 2H), 4.07 (s, 2H), 3.86 (dd, J=13.5, 7.2 Hz, 2H), 3.50-3.34 (m, 2H), 3.11 (d, J=13.9 Hz, 2H), 2.57-2.37 (m, 4H), 2.16-1.95 (m, 4H). MS (ESI, m/e) [M+H]⁺ 641.3.

### Example 161

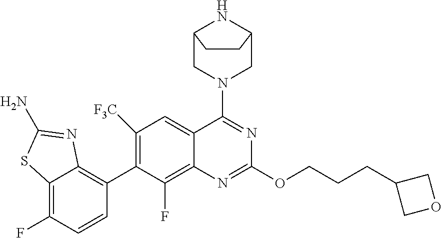 [SMILES: Nc1nc2c(-c3c(C(F)(F)F)cc4c(N5CC6CCC(C5)N6)nc(OCCCC5COC5)nc4c3F)ccc(F)c2s1]

Example 161 was prepared by similar procedure as described in Example 145 by replacing (tetrahydrofuran-3-yl)methanol with 3-(oxetan-3-yl)propan-1-ol to give the title product (6.05 mg, 14% yield for 2 steps). ¹H NMR (500 MHz, CD₃OD) δ 8.12 (s, 1H), 7.19 (dd, J=8.0, 5.7 Hz, 1H), 6.97 (t, J=8.8 Hz, 1H), 5.00-4.77 (m, 2H), 4.73-4.55 (m, 2H), 4.43 (dt, J=28.9, 6.2 Hz, 4H), 4.07 (s, 2H), 3.84 (dd, J=13.6, 6.2 Hz, 2H), 3.19-2.97 (m, 1H), 2.04 (t, J=7.6 Hz, 4H), 1.89 (dd, J=15.3, 7.5 Hz, 2H), 1.84-1.71 (m, 2H). MS (ESI, m/e) [M+H]⁺ 607.4.

### Example 162: (3-(((7-(2-amino-7-fluorobenzo[d]thiazol-4-yl)-4-(3,8-diaza bicyclo[3.2.1]octan-3-yl)-8-fluoro-6-(trifluoromethyl)quinazolin-2-yl)oxy)methyl)bicyclo[1.1.1] pentan-1-yl)methanol

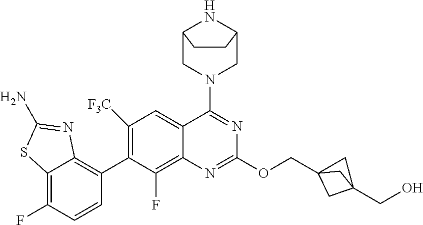 [SMILES: Nc1nc2c(-c3c(C(F)(F)F)cc4c(N5CC6CCC(C5)N6)nc(OCC56CC(CO)(C5)C6)nc4c3F)ccc(F)c2s1]

Example 162 was prepared by similar procedure as described in Example 145 by replacing (tetrahydrofuran-3-yl)methanol with bicyclo[1.1.1]pentane-1,3-diyldimethanol to give the title product (5.03 mg). 1H NMR (500 MHz, DMSO-d₆) δ 8.07 (s, 1H), 7.90 (s, 2H), 7.20-7.17 (m, 1H), 7.05-7.01 (m, 1H), 4.45-4.28 (m, 5H), 3.6-3.54 (m, 5H), 3.37-3.32 (m, 2H), 1.68-1.61 (m, 10H). MS (ESI, m/e) [M+H]⁺ 619.4.

### Example 163: 4-(2-((8-oxabicyclo[3.2.1]octan-3-yl)methoxy)-4-(3,8-diaza bicyclo[3.2.1]octan-3-yl)-8-fluoro-6-(trifluoromethyl)quinazolin-7-yl)-7-fluorobenzo[d]thiazol-2-amine

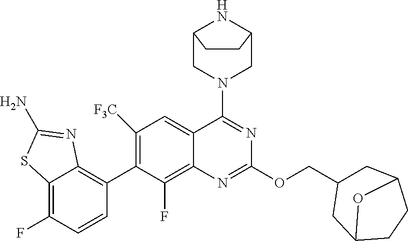 [SMILES: Nc1nc2c(-c3c(C(F)(F)F)cc4c(N5CC6CCC(C5)N6)nc(OCC5CC6CCC(C5)O6)nc4c3F)ccc(F)c2s1]

Example 163 was prepared by similar procedure as described in Example 145 by replacing (tetrahydrofuran-3-yl)methanol with (8-oxabicyclo[3.2.1]octan-3-yl)methanol to give the title product (11.76 mg, FA salt). 1H NMR (500 MHz, DMSO-d₆) δ 8.21 (s, 1H), 8.07 (s, 1H), 7.90 (s, 2H), 7.20-7.17 (m, 1H), 7.05-7.01 (m, 1H), 4.37-4.24 (m, 4H), 4.15-4.14 (m, 2H), 3.62-3.51 (m, 5H), 2.28-2.23 (m, 1H), 1.81-1.41 (m, 12H). MS (ESI, m/e) [M+H]⁺ 633.3.

### Example 164: 1-(3-(((7-(2-amino-7-fluorobenzo[d]thiazol-4-yl)-4-(3,8-diaza bicyclo[3.2.1]octan-3-yl)-8-fluoro-6-(trifluoromethyl)quinazolin-2-yl)oxy)methyl)azetidin-1-yl)ethan-1-one

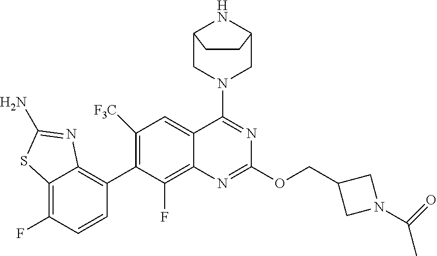 [SMILES: CC(=O)N1CC(COc2nc(N3CC4CCC(C3)N4)c3cc(C(F)(F)F)c(-c4ccc(F)c5sc(N)nc45)c(F)c3n2)C1]

Example 164 was prepared by similar procedure as described in Example 145 by replacing (tetrahydrofuran-3-yl)methanol with 1-(3-(hydroxymethyl)azetidin-1-yl)ethan-1-one to give the title product (11.9 mg). 1H NMR (500 MHz, DMSO-d₆) δ 8.07 (s, 1H), 7.91 (s, 2H), 7.19-7.18 (m, 1H), 7.06-7.02 (m, 1H), 4.53-4.48 (m, 3H), 4.38-4.37 (m, 1H), 4.24-4.21 (m, 1H), 3.97-3.93 (m, 4H), 3.81-3.65 (m, 3H), 3.04-3.02 (m, 1H), 1.83-1.74 (m, 7H). MS (ESI, m/e) [M+H]⁺ 620.3.

### Example 165: 4-(2-((2-oxabicyclo[2.2.2]octan-4-yl)methoxy)-4-(3,8-diaza bicyclo[3.2.1]octan-3-yl)-8-fluoro-6-(trifluoromethyl)quinazolin-7-yl)-7-fluorobenzo[d]thiazol-2-amine

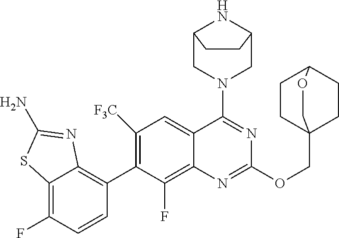 [SMILES: Nc1nc2c(-c3c(C(F)(F)F)cc4c(N5CC6CCC(C5)N6)nc(OCC56CCC(CC5)OC6)nc4c3F)ccc(F)c2s1]

Example 165 was prepared by similar procedure as described in Example 145 by replacing (tetrahydrofuran-3-yl)methanol with (2-oxabicyclo[2.2.2]octan-4-yl)methanol to give the title product (7.61 mg). 1H NMR (500 MHz, DMSO-d₆) δ 8.23 (s, 1H), 8.07 (s, 1H), 7.90 (s, 2H), 7.19-7.18 (m, 1H), 7.06-7.02 (m, 1H), 4.37-4.23 (m, 2H), 4.04-4.01 (m, 2H), 3.71-3.69 (m, 4H), 3.62-3.50 (m, 4H), 1.91-1.86 (m, 2H), 1.67-1.52 (m, 10H). MS (ESI, m/e) [M+H]⁺ 633.4.

### Example 166: 4-(4-(3,8-diazabicyclo[3.2.1]octan-3-yl)-8-fluoro-2-((2-methyl oxetan-2-yl)methoxy)-6-(trifluoromethyl)quinazolin-7-yl)-7-fluorobenzo[d]thiazol-2-amine

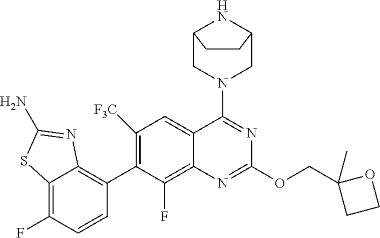 [SMILES: CC1(COc2nc(N3CC4CCC(C3)N4)c3cc(C(F)(F)F)c(-c4ccc(F)c5sc(N)nc45)c(F)c3n2)CCO1]

Synthetic Route

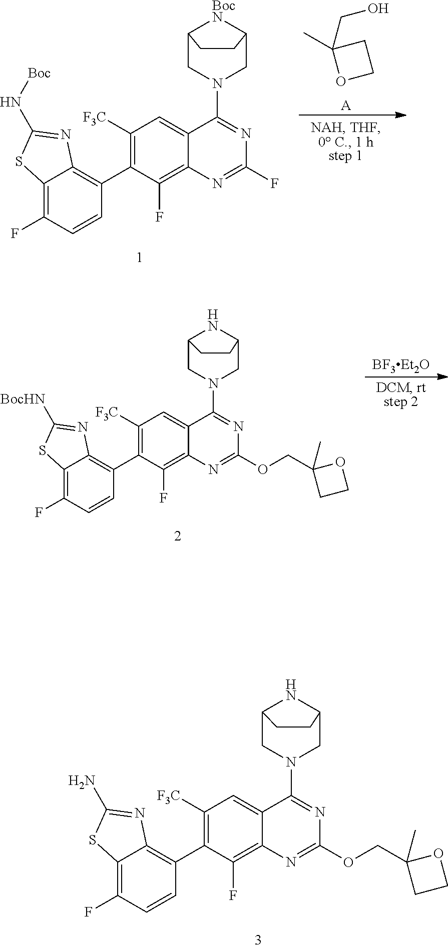

### Step 1: tert-butyl 3-(7-(2-((tert-butoxycarbonyl)amino)-7-fluorobenzo[d]thiazol-4-yl)-8-fluoro-2-((2-methyloxetan-2-yl)methoxy)-6-(trifluoromethyl)quinazolin-4-yl)-3,8-diaza bicyclo[3.2.1]octane-8-carboxylate

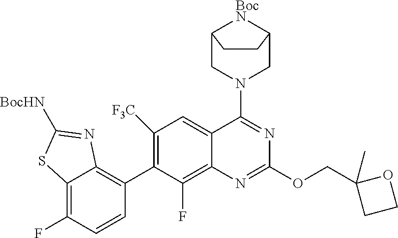 [SMILES: CC(C)(C)OC(=O)Nc1nc2c(-c3c(C(F)(F)F)cc4c(N5CC6CCC(C5)N6C(=O)OC(C)(C)C)nc(OCC5(C)CCO5)nc4c3F)ccc(F)c2s1]

A mixture of (2-methyloxetan-2-yl)methanol (50 mg, 0.49 mmol) in THF (5 mL) was cooled to 0° C. and was added NaH (16 mg, 0.39 mmol) one portion. The mixture was stirred at 0° C. for 30 min and the solution of tert-butyl 3-(7-(2-((tertbutoxy carbonyl)amino)-7-fluoro benzo[d]thiazol-4-yl)-2,8-difluoro-6(trifluoromethyl)quinazolin-4-yl)-3,8-diazabicyclo [3.2.1]octane-8-carboxylate (87 mg, 0.12 mmol) in THE was added dropwise at 0° C. The resulting mixture was stirred at 0° C. for 1 hour, then diluted with EA (100 mL), washed with brine (20 mL), repeated the operation 3 times, dried over anhydrous Na₂SO₄ and filtered. The filtrate was concentrated and purified by column chromatography to give the title product (62 mg, 64.5%). MS (ESI, m/e) [M+H]⁺ 793.6.

### Step 2: 4-(4-(3,8-diazabicyclo[3.2.1]octan-3-yl)-8-fluoro-2-((2-methyl oxetan-2-yl)methoxy)-6-(trifluoromethyl)quinazolin-7-yl)-7-fluorobenzo[d]thiazol-2-amine

To a solution of tert-butyl 3-(7-(2-((tert-butoxycarbonyl)amino)-7-fluoro benzo[d]thiazol-4-yl)-8-fluoro-2-((2-methyloxetan-2-yl)methoxy)-6-(trifluoromethyl) quinazolin-4-yl)-3,8-diazabicyclo[3.2.1]octane-8-carboxylate (20 mg, 0.025 mmol) in DCM (5 mL) was added 2 drops of BF₃-Et₂O. The mixture was stirred at room temperature for 2-3 hours and the resulting solution was concentrated and purified by pre-HPLC to give the title product (4 mg, FA salt, 26.7%). 1H NMR (500 MHz, CD₃OD) δ 8.13 (s, 1H), 7.32 (s, 1H), 7.24-7.08 (m, 1H), 6.97 (t, J=8.8 Hz, 1H), 4.69-4.57 (m, 3H), 4.08 (s, 2H), 3.86 (d, J=13.9 Hz, 2H), 3.69 (t, J=6.5 Hz, 2H), 3.27 (m, 2H), 2.29 (t, J=6.3 Hz, 2H), 2.05 (s, 4H), 1.83 (s, 3H). MS (ESI, m/e) [M+H]⁺ 593.4.

### Example 167:4-(4-(3,8-diazabicyclo[3.2.1]octan-3-yl)-8-fluoro-2-(3-methoxy cyclobutoxy)-6-(trifluoromethyl)quinazolin-7-yl)-7-fluorobenzo[d]thiazol-2-amine

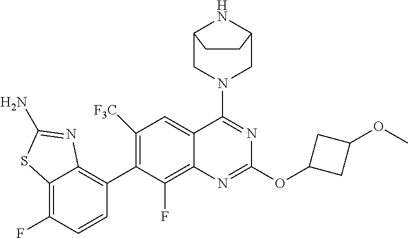 [SMILES: COC1CC(Oc2nc(N3CC4CCC(C3)N4)c3cc(C(F)(F)F)c(-c4ccc(F)c5sc(N)nc45)c(F)c3n2)C1]

Example 167 was prepared by similar procedure as described in Example 145 by replacing (tetrahydrofuran-3-yl)methanol with 3-methoxycyclobutan-1-ol to give the title product (4 mg, TFA salt, 16% yield). ¹H NMR (400 MHz, CD₃OD) δ 8.12 (s, 1H), 7.21-7.18 (m, 1H), 7.00-6.97 (m, 1H), 5.03-5.02 (m, 1H), 4.72-4.69 (m, 2H), 4.27-4.23 (m, 2H), 3.92-3.89 (m, 2H), 3.73-3.71 (m, 1H), 3.16 (s, 3H), 2.97-2.95 (m, 2H), 2.17-2.06 (m, 6H). MS (ESI, m/e) [M+H]⁺ 593.4.

### Example 168: 4-(4-(3,8-diazabicyclo[3.2.1]octan-3-yl)-8-fluoro-2-(((1S,2R)-2-methoxycyclobutyl)methoxy)-6-(trifluoromethyl)quinazolin-7-yl)-7-fluorobenzo[d]thiazol-2-amine

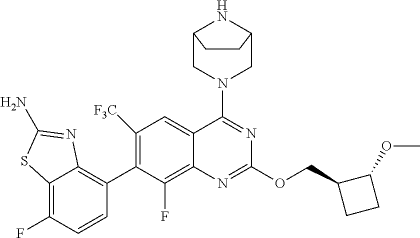 [SMILES: CO[C@@H]1CC[C@H]1COc1nc(N2CC3CCC(C2)N3)c2cc(C(F)(F)F)c(-c3ccc(F)c4sc(N)nc34)c(F)c2n1]

Example 168 was prepared by similar procedure as described in Example 145 by replacing (tetrahydrofuran-3-yl)methanol with ((1S,2R)-2-methoxycyclobutyl)methanol to give the title product (2 mg, 15% yield). ¹H NMR (400 MHz, CD₃OD) δ 8.11 (s, 1H), 7.20-7.17 (m, 1H), 6.98-6.94 (m, 1H), 4.54-4.44 (m, 4H), 3.85-3.72 (m, 5H), 3.28 (s, 3H), 2.69-2.64 (m, 1H), 2.22-2.18 (m, 1H), 1.94-1.75 (m, 6H), 1.53-1.46 (m, 1H). MS (ESI, m/e) [M+H]⁺ 607.5.

### Example 169: 3-((7-(2-amino-7-fluorobenzo[d]thiazol-4-yl)-4-(3,8-diazabicyclo [3.2.1]octan-3-yl)-8-fluoro-6-(trifluoromethyl)quinazolin-2-yl)oxy)-2-methoxy-2-(methoxy methyl)propan-1-ol

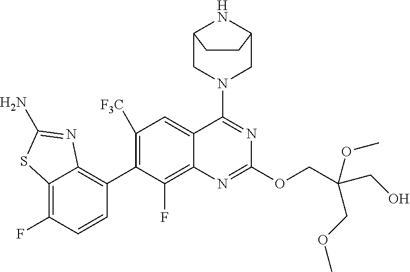 [SMILES: COCC(CO)(COc1nc(N2CC3CCC(C2)N3)c2cc(C(F)(F)F)c(-c3ccc(F)c4sc(N)nc34)c(F)c2n1)OC]

Example 169 was prepared by similar procedure as described in Example 166 by replacing (2-methyloxetan-2-yl)methanol with (3-methoxyoxetan-3-yl)methanol to give the title product (5 mg). ¹H NMR (500 MHz, CD₃OD) 1H NMR (500 MHz, CD₃OD) δ 8.13 (s, 1H), 7.32 (s, 1H), 7.26-7.15 (m, 1H), 6.98 (t, J=8.8 Hz, 1H), 4.65 (t, J=30.6 Hz, 3H), 4.08 (s, 2H), 3.87 (d, J=13.9 Hz, 2H), 3.69 (t, J=6.5 Hz, 2H), 3.27 (m, 2H), 2.30 (t, J=6.3 Hz, 2H), 2.05 (s, 4H), 1.83 (s, 3H). MS (ESI, m/e) [M+H]⁺ 641.4.

### Example 170: 4-(((7-(2-amino-7-fluorobenzo[d]thiazol-4-yl)-4-(3,8-diazabicyclo [3.2.1]octan-3-yl)-8-fluoro-6-(trifluoromethyl)quinazolin-2-yl)oxy)methyl)oxazolidin-2-one

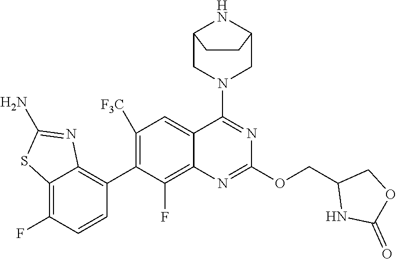 [SMILES: Nc1nc2c(-c3c(C(F)(F)F)cc4c(N5CC6CCC(C5)N6)nc(OCC5COC(=O)N5)nc4c3F)ccc(F)c2s1]

Example 170 was prepared by similar procedure as described in Example 145 by replacing (tetrahydrofuran-3-yl)methanol with 4-(hydroxymethyl)oxazolidin-2-one to give the title product (3.24 mg). 1H NMR (500 MHz, DMSO-d₆) δ 8.10 (s, 1H), 7.95-7.91 (m, 2H), 7.19-7.18 (m, 1H), 7.06-7.02 (m, 1H), 5.14-5.13 (m, 1H), 4.63-4.57 (m, 2H), 4.45-4.20 (m, 4H), 3.93-3.91 (m, 1H), 3.64-3.50 (m, 4H), 1.42-1.41 (m, 4H). MS (ESI, m/e) [M+H]⁺ 608.4.

### Example 171: 3-(((7-(2-amino-7-fluorobenzo[d]thiazol-4-yl)-4-(3,8-diazabicyclo [3.2.1]octan-3-yl)-8-fluoro-6-trifluoromethyl)quinazolin-2-yl)oxy)methyl)thietane 1,1-dioxide

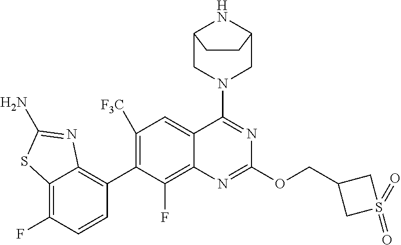 [SMILES: Nc1nc2c(-c3c(C(F)(F)F)cc4c(N5CC6CCC(C5)N6)nc(OCC5CS(=O)(=O)C5)nc4c3F)ccc(F)c2s1]

Example 171 was prepared by similar procedure as described in Example 166 by replacing (tetrahydrofuran-3-yl)methanol with 3-(hydroxymethyl)thietane 1,1-dioxide to give the title product (10.77 mg). 1H NMR (500 MHz, DMSO-d₆) δ 8.19 (s, 1H), 8.09 (s, 1H), 7.91 (s, 2H), 7.19-7.18 (m, 1H), 7.06-7.02 (m, 1H), 4.53-4.52 (m, 2H), 4.41-4.30 (m, 4H), 4.08-4.06 (m, 2H), 3.68-3.57 (m, 5H), 3.01-3.00 (m, 1H), 1.70-1.68 (m, 4H). MS (ESI, m/e) [M+H]⁺ 627.4.

### Example 172: 2-(3-(2-((7-(2-amino-7-fluorobenzo[d]thiazol-4-yl)-4-(3,8-diaza bicyclo[3.2.1]octan-3-yl)-8-fluoro-6-(trifluoromethyl)quinazolin-2-yl)oxy)ethyl)oxetan-3-yl) ethan-1-ol

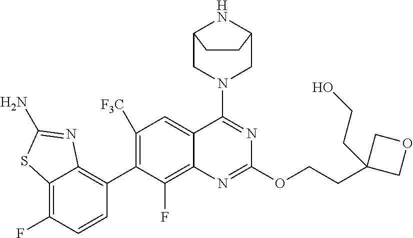 [SMILES: Nc1nc2c(-c3c(C(F)(F)F)cc4c(N5CC6CCC(C5)N6)nc(OCCC5(CCO)COC5)nc4c3F)ccc(F)c2s1]

Example 172 was prepared by similar procedure as described in Example 145 by replacing (tetrahydrofuran-3-yl)methanol with 2,2′-(oxetane-3,3-diyl)bis(ethan-1-ol) to give the title product (1 mg, 9% yield). 1H NMR (400 MHz, CD₃OD) δ 8.12 (s, 1H), 7.20-7.18 (m, 1H), 6.99-6.95 (m, 1H), 4.67-4.64 (m, 2H), 4.45-4.39 (m, 2H), 4.12-4.05 (m, 2H), 3.91-3.81 (m, 5H), 3.71-3.69 (m, 2H), 3.58-3.56 (m, 1H), 2.06-1.98 (m, 5H), 1.93-1.88 (m, 3H). MS (ESI, m/e) [M+H]⁺ 637.3.

### Example 173: 2-(3-(2-((7-(2-amino-7-fluorobenzo[d]thiazol-4-yl)-4-(3,8-diaza bicyclo[3.2.1]octan-3-yl)-8-fluoro-6-(trifluoromethyl)quinazolin-2-yl)oxy)ethyl)oxetan-3-yl) ethan-1-ol

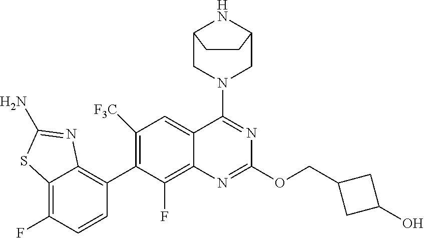 [SMILES: Nc1nc2c(-c3c(C(F)(F)F)cc4c(N5CC6CCC(C5)N6)nc(OCC5CC(O)C5)nc4c3F)ccc(F)c2s1]

Example 173 was prepared by similar procedure as described in Example 145 by replacing (tetrahydrofuran-3-yl)methanol with 3-(hydroxymethyl)cyclobutan-1-ol to give the title product (1 mg, 9% yield). 1H NMR (400 MHz, CD₃OD) δ 8.10 (s, 1H), 7.21-7.17 (m, 1H), 6.98-6.95 (m, 1H), 5.23-5.20 (m, 1H), 4.63-4.60 (m, 2H), 4.47-4.42 (m, 2H), 4.14-4.05 (m, 2H), 3.84-3.79 (m, 2H), 3.58-3.56 (m, 1H), 2.65-2.58 (m, 1H), 2.47-2.40 (m, 1H), 2.31-2.19 (m, 1H), 2.04-1.96 (m, 5H), 1.83-1.77 (m, 1H). MS (ESI, m/e) [M+H]⁺ 593.2.

### Example 174: 4-(4-(3,8-diazabicyclo[3.2.1]octan-3-yl)-8-fluoro-2-((1-methyl-2-oxabicyclo[2.1.1]hexan-4-yl)methoxy)-6-(trifluoromethyl)quinazolin-7-yl)-7-fluorobenzo[d] thiazol-2-amine

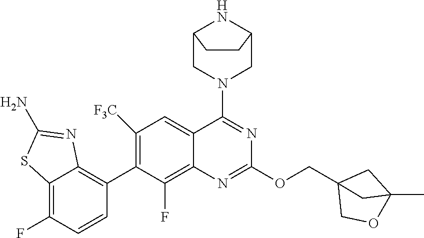 [SMILES: CC12CC(COc3nc(N4CC5CCC(C4)N5)c4cc(C(F)(F)F)c(-c5ccc(F)c6sc(N)nc56)c(F)c4n3)(CO1)C2]

Example 174 was prepared by similar procedure as described in Example 145 by replacing (tetrahydrofuran-3-yl)methanol with (1-methyl-2-oxabicyclo[2.1.1]hexan-4-yl) methanol to give the title product (4 mg, 16% yield). ¹H NMR (400 MHz, CD₃OD) δ 8.12 (s, 1H), 7.20-7.17 (m, 1H), 6.99-6.95 (m, 1H), 4.76-4.71 (m, 2H), 4.66-4.60 (m, 2H), 4.08-3.99 (m, 2H), 3.84-3.78 (m, 4H), 2.07-2.00 (m, 4H), 1.83-1.78 (m, 2H), 1.63-1.61 (m, 2H), 1.43 (s, 3H). MS (ESI, m/e) [M+H]⁺ 619.5.

### Example 175: 4-(4-(3,8-diazabicyclo[3.2.1]octan-3-yl)-8-fluoro-2-((1-(methyl sulfonyl)azetidin-3-yl)methoxy)-6-(trifluoromethyl)quinazolin-7-yl)-7-fluorobenzo[d]thiazol-2-amine

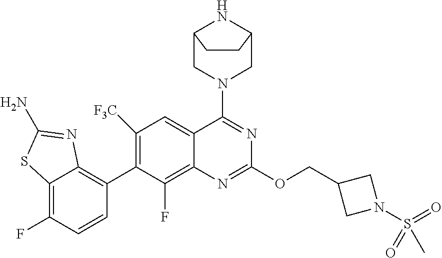 [SMILES: CS(=O)(=O)N1CC(COc2nc(N3CC4CCC(C3)N4)c3cc(C(F)(F)F)c(-c4ccc(F)c5sc(N)nc45)c(F)c3n2)C1]

Example 175 was prepared by similar procedure as described in Example 166 by replacing (2-methyloxetan-2-yl)methanol with (1-(methylsulfonyl)azetidin-3-yl)methanol to give the title product (10 mg). ¹H NMR (500 MHz, CD₃OD) δ 8.13 (s, 1H), 7.32-7.06 (m, 1H), 6.97 (t, J=8.8 Hz, 1H), 4.72-4.41 (m, 4H), 4.19 (s, 2H), 4.07 (t, J=8.3 Hz, 2H), 4.01-3.78 (m, 4H), 3.16-3.12 (m, 1H), 2.99 (s, 3H), 2.11-2.03 (m, 4H). MS (ESI, m/e) [M+H]⁺ 656.4.

### Example 176: 4-(4-(3,8-diazabicyclo[3.2.1]octan-3-yl)-8-fluoro-2-((3-methoxyoxetan-3-yl)methoxy)-6-(trifluoromethyl)quinazolin-7-yl)-7-fluorobenzo[d] thiazol-2-amine

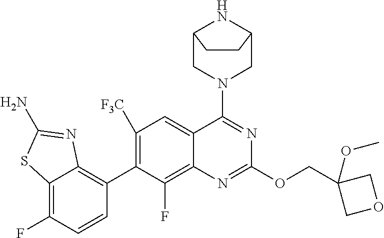 [SMILES: COC1(COc2nc(N3CC4CCC(C3)N4)c3cc(C(F)(F)F)c(-c4ccc(F)c5sc(N)nc45)c(F)c3n2)COC1]

Example 176 was prepared by similar procedure as described in Example 166 by replacing (2-methyloxetan-2-yl)methanol with (3-methoxyoxetan-3-yl)methanol to give the title product (5 mg). ¹H NMR (500 MHz, DMSO-d₆) δ 8.09 (s, 1H), 7.91 (s, 2H), 7.27-7.10 (m, 1H), 7.04 (t, J=8.8 Hz, 1H), 4.75-4.69 (m, 2H), 4.54 (dd, J=49.0, 7.0 Hz, 3H), 4.40 (d, J=11.5 Hz, 1H), 3.64-4.26 (m, 1H), 3.72-3.44 (m, 5H), 3.30 (d, J=15.3 Hz, 4H), 1.65 (s, 4H). MS (ESI, m/e) [M+H]⁺ 609.4.

### Example 177: 3-((7-(2-amino-7-fluorobenzo[d]thiazol-4-yl)-4-(3,8-diazabicyclo [3.2.1]octan-3-yl)-8-fluoro-6-(trifluoromethyl)quinazolin-2-yl)oxy)cyclobutan-1-ol

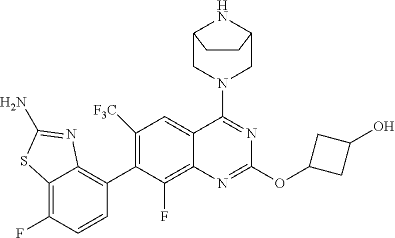 [SMILES: Nc1nc2c(-c3c(C(F)(F)F)cc4c(N5CC6CCC(C5)N6)nc(OC5CC(O)C5)nc4c3F)ccc(F)c2s1]

Example 177 was prepared by similar procedure as described in Example 145 by replacing cyclobutane-1,1-diyldimethanol with cyclobutane-1,3-diol to give the title product (4 mg, FA salt, 15% yield). ¹H NMR (400 MHz, CD₃OD) δ 8.45 (s, 1H), 8.10 (s, 1H), 7.20-7.17 (m, 1H), 6.98-6.95 (m, 1H), 4.92-4.89 (m, 1H), 4.61-4.58 (m, 2H), 4.03-4.00 (m, 3H), 3.79-3.77 (m, 2H), 2.97-2.93 (m, 2H), 2.15-2.09 (m, 2H), 2.05-1.95 (m, 4H). MS (ESI, m/e) [M+H]⁺ 579.3.

### Example 178: 3-(((7-(2-amino-7-fluorobenzo[d]thiazol-4-yl)-4-(3,8-diazabicyclo [3.2.1]octan-3-yl)-6-chloro-8-fluoroquinazolin-2-yl)oxy)methyl)-1-methylpiperidine-3-carbonitrile

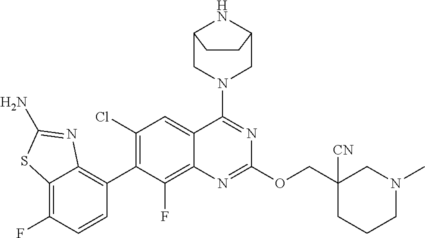 [SMILES: CN1CCCC(C#N)(COc2nc(N3CC4CCC(C3)N4)c3cc(Cl)c(-c4ccc(F)c5sc(N)nc45)c(F)c3n2)C1]

Synthetic Route

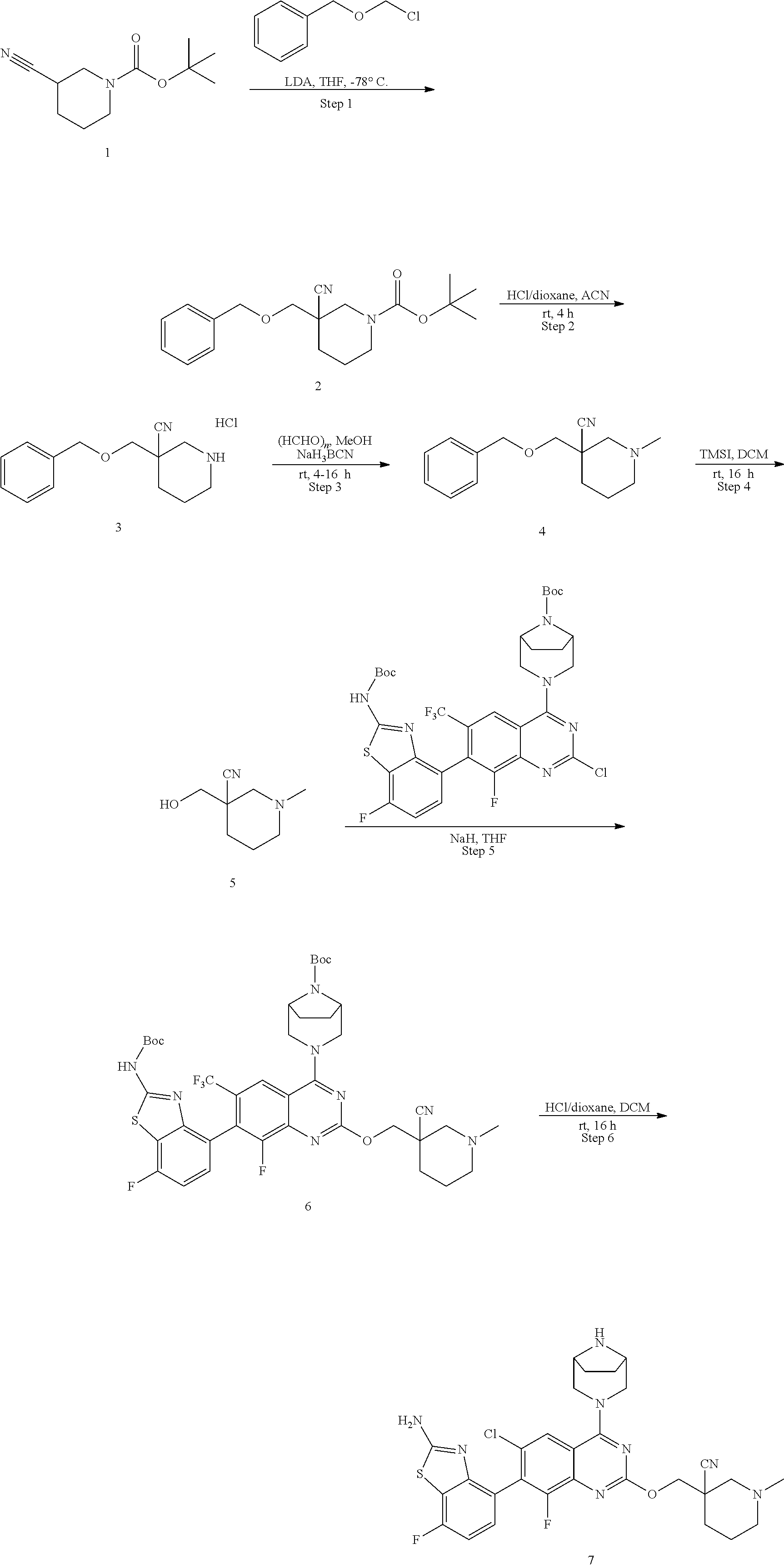

### Step 1: tert-butyl 3-((benzyloxy)methyl)-3-cyanopiperidine-1-carboxylate

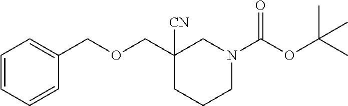 [SMILES: CC(C)(C)OC(=O)N1CCCC(C#N)(COCc2ccccc2)C1]

To a solution of tert-butyl 3-cyanopiperidine-1-carboxylate (4.2 g, 20 mmol) in THF (200 mL) was added LDA (2M in THF, 11 mL) at −65° C. After stirring for 1 hour, the ((chloromethoxy)methyl)benzene (3.45 g, 22 mmol) was added drop-wise and stirred for 5 hours at −60° C. The resulting solution was quenched by saturated NH₄Cl aqueous solution (100 mL) and exacted with EA (100 mL) for 3 times. The combined EA phase was dried over anhydrous Na₂SO₄ and concentrated to give a residue which was further purified by combi-flash (PE/EA=2/1) to give the title product (6.8 g, 100% yield). MS (ESI, m/e) [M+Na]⁺353.1.

### Step 2: 3-((benzyloxy)methyl)piperidine-3-carbonitrile

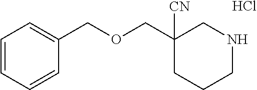 [SMILES: Cl ; N#CC1(COCc2ccccc2)CCCNC1]

A solution of tert-butyl 3-((benzyloxy)methyl)-3-cyanopiperidine-1-carboxylate (3.4 g, 10 mmol) in ACN (60 mL) was added 4M HCl/1,4-dioxane (20 mL) and stirred at room temperature for 4 hours. The resulting reaction solution was concentrated to give the title product (3 g, 100% yield). MS (ESI, m/e) [M+H]⁺ 231.1.

### Step 3: 3-((benzyloxy)methyl)-1-methylpiperidine-3-carbonitrile

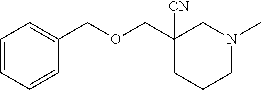 [SMILES: CN1CCCC(C#N)(COCc2ccccc2)C1]

To a mixture of 3-((benzyloxy)methyl)piperidine-3-carbonitrile (3 g, 10 mmol) in MeCN (50 mL) and MeOH (100 mL) was added DIEA (4 g, 30 mmol), (HCHO)ₙ (3.16 g, 105 mmol) and NaBH₃CN (1.99 g, 33 mmol). The mixture was stirred at room temperature for 6 hours. Then, the reaction mixture was concentrated to give the residue. The residue was purified by combi-flash (DCM/MeOH=10/1) to give the title product (1.84 g, 74% yield). MS (ESI, m/e) [M+H]⁺ 245.3.

### Step 4: 3-(hydroxymethyl)-1-methylpiperidine-3-carbonitrile

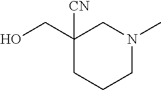

A solution of 3-((benzyloxy)methyl)-1-methylpiperidine-3-carbonitrile (70 mg, 0.29 mmol) in DCM (4 mL) was added TMSI (200 mg, 1 mmol) and stirred at room temperature for 24 hours. The resulting solution was concentrated and treated with Na₂CO₃/MeOH. After filtering, the filtrate was concentrated to give the residue. The residue was purified by combi-flash (DCM/MeOH=10/1) to give the title product (37 mg, 77% yield). MS (ESI, m/e) [M+H]⁺ 155.3.

### Step 5: tert-butyl 3-(7-(2-((tert-butoxycarbonyl)amino)-7-fluorobenzo[d]thiazol-4-yl)-2-((3-cyano-1-methylpiperidin-3-yl)methoxy)-8-fluoro-6-(trifluoromethyl)quinazolin-4-yl)-3,8-diazabicyclo[3.2.1]octane-8-carboxylate

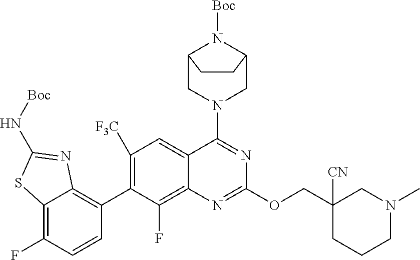 [SMILES: CN1CCCC(C#N)(COc2nc(N3CC4CCC(C3)N4C(=O)OC(C)(C)C)c3cc(C(F)(F)F)c(-c4ccc(F)c5sc(NC(=O)OC(C)(C)C)nc45)c(F)c3n2)C1]

To a solution of 3-(hydroxymethyl)-1-methylpiperidine-3-carbonitrile (37 mg, 0.24 mmol) in THF (8 mL) was added 60% NaH (20 mg, 0.5 mmol) and stirred at 0° C. for 0.5 hour. The resulting solution was added tert-butyl 3-(7-(2-((tert-butoxycarbonyl)amino)-7-fluorobenzo[d]thiazol-4-yl)-2-chloro-8-fluoro-6-(trifluoromethyl)quinazolin-4-yl)-3,8-diaza bicyclo[3.2.1]octane-8-carboxylate (18 mg, 0.025 mmol) and stirred at room temperature for 16 hours. The resulting solution was added water (20 mL) and extracted with EA (10 mL) for 3 times. The combined EA phase was dried over anhydrous Na₂SO₄ and concentrated to give the title product (22 mg, 100% yield). MS (ESI, m/e) [M+H]⁺ 845.5.

### Step 6: 3-(((7-(2-amino-7-fluorobenzo[d]thiazol-4-yl)-4-(3,8-diazabicyclo[3.2.1] octan-3-yl)-6-chloro-8-fluoroquinazolin-2-yl)oxy)methyl)-1-methylpiperidine-3-carbonitrile

A solution of tert-butyl 3-(7-(2-((tert-butoxycarbonyl)amino)-7-fluorobenzo[d] thiazol-4-yl)-2-((3-cyano-1-methylpiperidin-3-yl)methoxy)-8-fluoro-6-(trifluoromethyl) quinazolin-4-yl)-3,8-diazabicyclo[3.2.1]octane-8-carboxylate (22 mg, 0.026 mmol) in DCM (2.5 mL) and MeCN (2.5 mL) was added 4M HCl/1,4-dioxane (2.5 mL). The mixture was stirred at room temperature for 16 hours. The resulting reaction solution was concentrated and purified by Prep-HPLC to give the title product (7.4 mg, FA salt). 1H NMR (500 MHz, CD₃OD) δ 8.48 (s, 1H), 8.14 (s, 1H), 7.21-7.18 (m, 1H), 6.99-6.95 (m, 1H), 4.68-4.64 (m, 3H), 4.53-4.49 (m, 1H), 4.04 (s, 2H), 3.86-3.82 (m, 2H), 3.19-3.16 (m, 1H), 2.81-2.79 (m, 1H), 2.31 (s, 3H), 2.23-2.21 (m, 1H), 2.10-2.03 (m, 6H), 1.94-1.87 (m, 1H), 1.82-1.79 (m, 1H), 1.59-1.55 (m, 1H). MS (ESI, m/e) [M+H]⁺ 645.2.

### Example 179: (6R)-6-(((7-(2-amino-7-fluorobenzo[d]thiazol-4-yl)-4-(3,8-diaza bicyclo[3.2.1]octan-3-yl)-8-fluoro-6-(trifluoromethyl)quinazolin-2-yl)oxy)methyl)morpholin-3-one

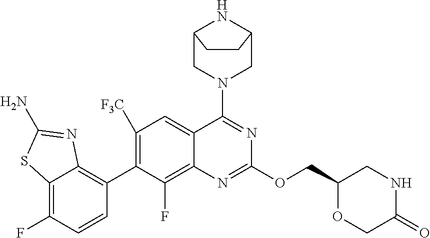 [SMILES: Nc1nc2c(-c3c(C(F)(F)F)cc4c(N5CC6CCC(C5)N6)nc(OC[C@H]5CNC(=O)CO5)nc4c3F)ccc(F)c2s1]

Example 179 was prepare by similar procedure as escribed in Example 166 by replacing (2-methyloxetan-2-yl)methanol with (R)-6-(hydroxymethyl)morpholin-3-one to give the title product (6 mg). ¹H NMR (500 MHz, DMSO-d₆) δ 8.08 (s, 1H), 8.03 (s, 1H), 7.91 (s, 2H), 7.26-7.13 (m, 1H), 7.04 (t, J=8.8 Hz, 1H), 4.54-4.38 (m, 3H), 4.32 (d, J=12.6 Hz, 1H), 4.18-4.01 (m, 3H), 3.89-3.64 (m, 3H), 3.58 (d, J=12.4 Hz, 1H), 3.24 (d, J=11.9 Hz, 2H), 2.54 (s, 1H), 1.70 (s, 4H). MS (ESI, m/e) [M+H]⁺ 622.2.

### Example 180: 4-(4-(3,8-diazabicyclo[3.2.1]octan-3-yl)-2-((3-(1,1-difluoroethyl) oxetan-3-yl)methoxy)-8-fluoro-6-(trifluoromethyl)quinazolin-7-yl)-7-fluorobenzo[d]thiazol-2-amine

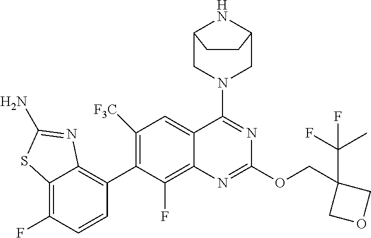 [SMILES: CC(F)(F)C1(COc2nc(N3CC4CCC(C3)N4)c3cc(C(F)(F)F)c(-c4ccc(F)c5sc(N)nc45)c(F)c3n2)COC1]

Example 180 was prepared by similar procedure as described in Example 166 by replacing (2-methyloxetan-2-yl)methanol with (3-(1,1-difluoroethyl)oxetan-3-yl)methanol to give the title product (9 mg). ¹H NMR (500 MHz, CD₃OD) δ 8.14 (s, 1H), 7.32-7.10 (m, 1H), 6.97 (t, J=8.8 Hz, 1H), 4.81 (s, 2H), 4.78 (d, J=6.6 Hz, 2H), 4.70 (d, J=6.4 Hz, 2H), 4.62 (t, J=12.5 Hz, 2H), 3.95 (s, 2H), 3.83-3.79 (m, 2H), 1.97 (t, J=9.5 Hz, 4H), 1.73 (t, J=19.4 Hz, 3H). MS (ESI, m/e) [M+H]⁺ 643.3.

### Example 181: 4-(4-(3,8-diazabicyclo[3.2.1]octan-3-yl)-8-fluoro-2-((3-methyl tetrahydrofuran-3-yl)methoxy)-6-(trifluoromethyl)quinazolin-7-yl)-7-fluorobenzo[d]thiazol-2-amine

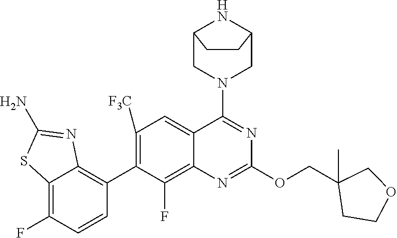 [SMILES: CC1(COc2nc(N3CC4CCC(C3)N4)c3cc(C(F)(F)F)c(-c4ccc(F)c5sc(N)nc45)c(F)c3n2)CCOC1]

Example 181 was prepared by similar procedure as described in Example 145 by replacing (tetrahydrofuran-3-yl)methanol with (3-methyltetrahydrofuran-3-yl)methanol to give the title product (14.40 mg). 1H NMR (500 MHz, CD₃OD) δ 8.11 (s, 1H), 7.19-7.18 (m, 1H), 6.97-6.95 (m, 1H), 4.58-4.56 (m, 2H), 4.36-4.34 (m, 2H), 3.91-3.84 (m, 4H), 3.79-3.75 (m, 3H), 3.49-3.47 (m, 1H), 2.04-2.00 (m, 5H), 1.97-1.93 (m, 1H), 1.27 (s, 3H). MS (ESI, m/e) [M+H]⁺ 607.4.

### Example 182: 4-(4-(3,8-diazabicyclo[3.2.1]octan-3-yl)-8-fluoro-2-((4-methyltetra hydro-2H-pyran-4-yl)methoxy)-6-(trifluoromethyl)quinazolin-7-yl)-7-fluorobenzo[d]thiazol-2-amine

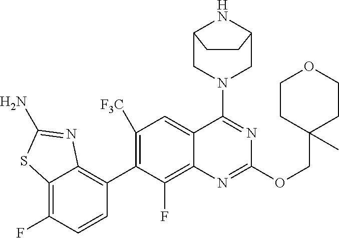 [SMILES: CC1(COc2nc(N3CC4CCC(C3)N4)c3cc(C(F)(F)F)c(-c4ccc(F)c5sc(N)nc45)c(F)c3n2)CCOCC1]

Example 182 was prepared by similar procedure as described in Example 145 by replacing (tetrahydrofuran-3-yl)methanol with (4-methyltetrahydro-2H-pyran-4-yl)methanol to give the title product (8.35 mg). 1H NMR (500 MHz, CD₃OD) δ 8.11 (s, 1H), 7.19-7.18 (m, 1H), 6.97-6.95 (m, 1H), 4.58-4.56 (m, 2H), 4.28 (s, 2H), 3.77-3.76 (m, 2H), 3.75-3.70 (m, 6H), 1.97-1.93 (m, 4H), 1.75-1.72 (m, 2H), 1.47-1.44 (m, 2H), 1.19 (s, 3H). MS (ESI, m/e) [M+H]⁺ 621.4.

### Example 183: 4-(4-(3,8-diazabicyclo[3.2.1]octan-3-yl)-8-fluoro-2-((3-methyltetra hydro-2H-pyran-3-yl)methoxy)-6-(trifluoromethyl)quinazolin-7-yl)-7-fluorobenzo[d]thiazol-2-amine

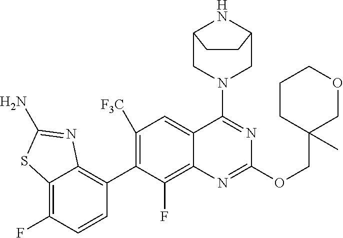 [SMILES: CC1(COc2nc(N3CC4CCC(C3)N4)c3cc(C(F)(F)F)c(-c4ccc(F)c5sc(N)nc45)c(F)c3n2)CCCOC1]

Example 183 was prepared by similar procedure as described in Example 145 by replacing (tetrahydrofuran-3-yl)methanol with (3-methyltetrahydro-2H-pyran-3-yl)methanol to give the title product (5.36 mg). 1H NMR (500 MHz, CD₃OD) δ 8.11 (s, 1H), 7.19-7.18 (m, 1H), 6.97-6.95 (m, 1H), 4.58-4.56 (m, 2H), 4.37 (s, 2H), 3.81-3.68 (m, 8H), 1.93-1.88 (m, 5H), 1.70-1.64 (m, 2H), 1.49-1.48 (m, 1H), 1.06 (s, 3H). MS (ESI, m/e) [M+H]⁺ 621.5.

### Example 184: 4-(4-(3,8-diazabicyclo[3.2.1]octan-3-yl)-8-fluoro-2-(2-(pyridin-3-yl)ethoxy)-6-(trifluoromethyl)quinazolin-7-yl)-7-fluorobenzo [d]thiazol-2-amine

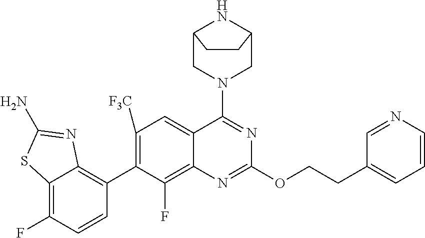 [SMILES: Nc1nc2c(-c3c(C(F)(F)F)cc4c(N5CC6CCC(C5)N6)nc(OCCc5cccnc5)nc4c3F)ccc(F)c2s1]

Example 184 was prepared by similar procedure as described in Example 145 by replacing (tetrahydrofuran-3-yl)methanol with 2-(pyridin-3-yl)ethan-1-ol to give the title product (8 mg, FA salt, 17% yield). 1H NMR (400 MHz, CD₃OD) δ 8.51 (s, 1H), 8.48 (s, 1H), 8.38 (d, J=4.7 Hz, 1H), 8.09 (s, 1H), 7.85 (d, J=7.8 Hz, 1H), 7.44-7.31 (m, 1H), 7.29-7.12 (m, 1H), 6.96 (t, J=8.8 Hz, 1H), 4.72 (t, J=6.3 Hz, 2H), 4.58 (t, J=13.4 Hz, 2H), 3.97 (s, 2H), 3.77 (t, J=11.8 Hz, 2H), 3.19 (t, J=6.3 Hz, 2H), 2.11-1.83 (m, 4H). MS (ESI, m/e) [M+H]⁺ 614.4.

### Example 185: 4-(2-(2-(1H-pyrazol-1-yl)ethoxy)-4-(3,8-diazabicyclo[3.2.1]octan-3-yl)-8-fluoro-6-(trifluoromethyl)quinazolin-7-yl)-7-fluorobenzo[d]thiazol-2-amine

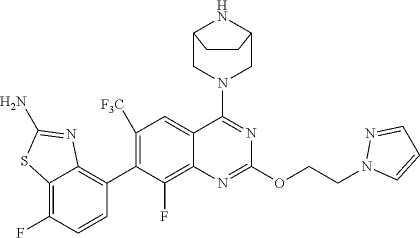 [SMILES: Nc1nc2c(-c3c(C(F)(F)F)cc4c(N5CC6CCC(C5)N6)nc(OCCn5cccn5)nc4c3F)ccc(F)c2s1]

Example 185 was prepared by similar procedure as described in Example 166 by replacing (2-methyloxetan-2-yl)methanol with 2-(1H-pyrazol-1-yl)ethan-1-ol to give the title product (8 mg). ¹H NMR (500 MHz, CD₃OD) δ 8.10 (s, 1H), 7.71 (s, 1H), 7.48 (s, 1H), 7.24-7.09 (m, 1H), 6.96 (t, J=8.8 Hz, 1H), 6.25 (s, 1H), 4.81 (s, 2H), 4.60 (dd, J=14.3, 9.5 Hz, 4H), 3.98 (s, 2H), 3.80 (t, J=11.9 Hz, 2H), 2.00 (s, 4H). MS (ESI, m/e) [M+H]⁺ 603.4.

### Example 186: 3-(((7-(2-amino-7-fluorobenzo[d]thiazol-4-yl)-4-(3,8-diazabicyclo [3.2.1]octan-3-yl)-8-fluoro-6-(trifluoromethyl)quinazolin-2-yl)oxy)methyl)bicyclo[1.1.1] pentane-1-carbonitrile

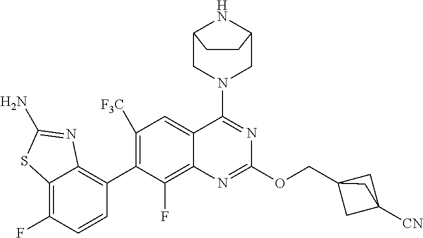 [SMILES: N#CC12CC(COc3nc(N4CC5CCC(C4)N5)c4cc(C(F)(F)F)c(-c5ccc(F)c6sc(N)nc56)c(F)c4n3)(C1)C2]

Example 186 was prepare by similar procedure as described in Example 145 by replacing (tetrahydrofuran-3-yl)methanol with 3-(hydroxymethyl)bicyclo[1.1.1]pentane-1-carbonitrile to give the title product (2 mg, 11% yield). 1H NMR (400 MHz, CD₃OD) δ 8.11 (s, 1H), 7.20-7.17 (m, 1H), 6.99-6.95 (m, 1H), 4.66-4.61 (m, 2H), 4.48-4.30 (m, 2H), 4.14-4.06 (m, 2H), 3.85-3.80 (m, 2H), 2.31 (s, 6H), 2.08-2.03 (m, 4H). MS (ESI, m/e) [M+H]⁺ 614.6.

### Example 187: (4-(((7-(2-amino-7-fluorobenzo[d]thiazol-4-yl)-4-(3,8-diaza bicyclo[3.2.1]octan-3-yl)-8-fluoro-6-(trifluoromethyl)quinazolin-2-yl)oxy)methyl)tetrahydro-2H-pyran-4-yl)methanol

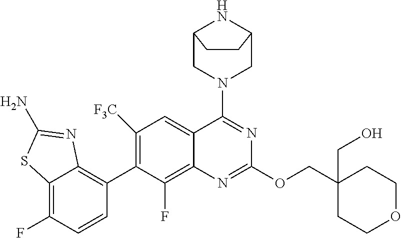 [SMILES: Nc1nc2c(-c3c(C(F)(F)F)cc4c(N5CC6CCC(C5)N6)nc(OCC5(CO)CCOCC5)nc4c3F)ccc(F)c2s1]

Example 187 was prepared by similar procedure as described in Example 145 by replacing (tetrahydrofuran-3-yl)methanol with (tetrahydro-2H-pyran-4,4-diyl)dimethanol to give the title product (3 mg, FA salt, 10% yield). ¹H NMR (400 MHz, CD₃OD) δ 8.45 (s, 1H), 8.12 (s, 1H), 7.19-7.17 (m, 1H), 6.99-6.97 (m, 1H), 4.67-4.62 (m, 3H), 4.51-4.47 (m, 2H), 4.08-3.98 (m, 2H), 3.84-3.79 (m, 2H), 3.76-3.70 (m, 3H), 3.64-3.61 (m, 2H), 2.03-1.88 (m, 6H), 1.74-1.60 (m, 2H). MS (ESI, m/e) [M+H]⁺ 637.4. MS (ESI, m/e) [M+H]⁺ 637.4.

### Example 188: 5-(2-((7-(2-amino-7-fluorobenzo[d]thiazol-4-yl)-4-(3,8-diazabicyclo[3.2.1]octan-3-yl)-8-fluoro-6-(trifluoromethyl)quinazolin-2-yl)oxy)ethyl) nicotinonitrile

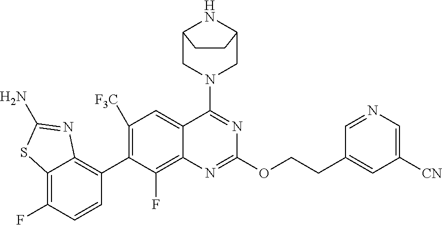 [SMILES: N#Cc1cncc(CCOc2nc(N3CC4CCC(C3)N4)c3cc(C(F)(F)F)c(-c4ccc(F)c5sc(N)nc45)c(F)c3n2)c1]

Example 188 was prepared by similar procedure as described in Example 145 by replacing (tetrahydrofuran-3-yl)methanol with 5-(2-hydroxyethyl)nicotinonitrile to give the title product (5.71 mg). ¹H NMR (500 MHz, CD₃OD) δ 8.79 (s, 1H), 8.75 (s, 1H), 8.22 (s, 1H), 8.10 (s, 1H), 7.23-7.15 (m, 1H), 6.98-6.94 (m, 1H), 4.74 (t, J=6.0 Hz, 2H), 4.58-4.51 (m, 2H), 3.86-3.82 (m, 2H), 3.78-3.68 (m, 2H), 3.22-3.18 (m, 2H), 1.95-1.85 (m, 4H). MS (ESI, m/e) [M+H]⁺ 639.5.

### Example 189: 4-(4-(3,8-diazabicyclo[3.2.1]octan-3-yl)-8-fluoro-2-(2-(oxetan-3-yl)ethoxy)-6-(trifluoromethyl)quinazolin-7-yl)-7-fluorobenzo[d]thiazol-2-amine

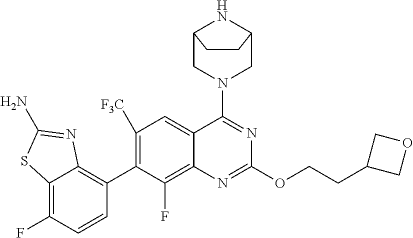 [SMILES: Nc1nc2c(-c3c(C(F)(F)F)cc4c(N5CC6CCC(C5)N6)nc(OCCC5COC5)nc4c3F)ccc(F)c2s1]

Example 189 was prepared by similar procedure as described in Example 145 by replacing (tetrahydrofuran-3-yl)methanol with 2-(oxetan-3-yl)ethan-1-ol to give the title product (7.47 mg). ¹H NMR (500 MHz, CD₃OD) δ 8.11 (s, 1H), 7.24-7.15 (m, 1H), 6.98-6.95 (m, 1H), 4.66-4.43 (m, 7H), 3.98-3.94 (m, 3H), 3.80-3.76 (m, 2H), 2.23-2.19 (m, 1H), 2.08-1.95 (m, 6H). MS (ESI, m/e) [M+H]⁺ 593.4.

### Example 190: (1-(((7-(2-amino-7-fluorobenzo[d]thiazol-4-yl)-4-(3,8-diaza bicyclo[3.2.1]octan-3-yl)-8-fluoro-6-(trifluoromethyl)quinazolin-2-yl)oxy)methyl)cyclo pentyl)methanol

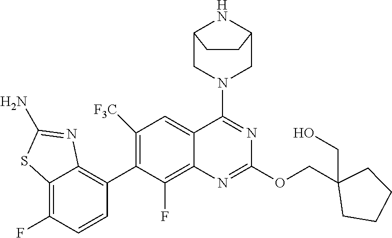 [SMILES: Nc1nc2c(-c3c(C(F)(F)F)cc4c(N5CC6CCC(C5)N6)nc(OCC5(CO)CCCC5)nc4c3F)ccc(F)c2s1]

Example 190 was prepared by similar procedure as described in Example 166 by replacing (2-methyloxetan-2-yl)methanol with cyclopentane-1,1-diyldimethanol to give the title product (27 mg). ¹H NMR (500 MHz, DMSO-d₆) δ 8.06 (s, 1H), 7.90 (s, 2H), 7.25-7.12 (m, 1H), 7.04 (t, J=8.8 Hz, 1H), 4.72-4.61 (m, 1H), 4.47 (d, J=12.9 Hz, 1H), 4.35 (d, J=12.9 Hz, 1H), 4.22 (s, 2H), 3.98-3.81 (m, 3H), 3.73 (d, J=13.0 Hz, 1H), 3.63 (d, J=13.0 Hz, 1H), 3.22 (d, J=4.9 Hz, 1H), 2.54 (s, 2H), 1.81 (s, 3H), 1.64-1.24 (m, 8H). MS (ESI, m/e) [M+H]⁺ 621.4.

### Example 191: (1-(((7-(2-amino-7-fluorobenzo[d]thiazol-4-yl)-4-(3,8-diaza bicyclo[3.2.1]octan-3-yl)-8-fluoro-6-(trifluoromethyl)quinazolin-2-yl)oxy)methyl)cyclohexyl) methanol

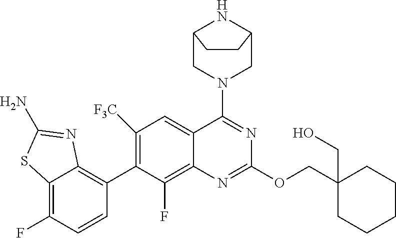 [SMILES: Nc1nc2c(-c3c(C(F)(F)F)cc4c(N5CC6CCC(C5)N6)nc(OCC5(CO)CCCCC5)nc4c3F)ccc(F)c2s1]

Example 191 was prepared by similar procedure as described in Example 166 by replacing (2-methyloxetan-2-yl)methanol with cyclohexane-1,1-diyldimethanol to give the title product (6 mg). ¹H NMR (500 MHz, CD₃OD) δ 8.11 (s, 1H), 7.31-7.09 (m, 1H), 6.97 (t, J=8.9 Hz, 1H), 4.65 (t, J=13.1 Hz, 2H), 4.39 (s, 2H), 4.08 (s, 2H), 3.92-3.77 (m, 2H), 3.55 (s, 2H), 2.06 (s, 4H), 1.52-1.49 (m, 10H). MS (ESI, m/e) [M+H]⁺ 635.5.

### Example 192: 1-(((7-(2-amino-7-fluorobenzo[d]thiazol-4-yl)-4-(3,8-diazabicyclo [3.2.1]octan-3-yl)-8-fluoro-6-(trifluoromethyl)quinazolin-2-yl)oxy)methyl)cyclopentane-1-carbonitrile

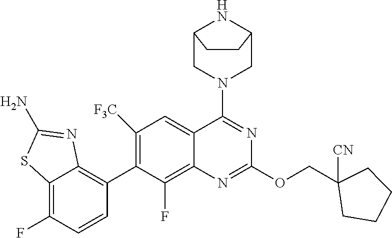 [SMILES: N#CC1(COc2nc(N3CC4CCC(C3)N4)c3cc(C(F)(F)F)c(-c4ccc(F)c5sc(N)nc45)c(F)c3n2)CCCC1]

Example 192 was prepared by similar procedure as described in Example 166 by replacing (2-methyloxetan-2-yl)methanol with 1-(hydroxymethyl)cyclopentane-1-carbonitrile to give the title product (6 mg). ¹H NMR (500 MHz, CD₃OD) δ 8.14 (s, 1H), 7.34-7.12 (m, 1H), 6.97 (t, J=8.8 Hz, 1H), 4.71 (d, J=13.2 Hz, 2H), 4.60-4.48 (m, 2H), 4.23 (s, 2H), 3.96 (s, 2H), 2.25-2.07 (m, 6H), 2.00-1.93 (m, 2H), 1.88-1.84 (m, 4H). MS (ESI, m/e) [M+H]⁺ 616.4.

### Example 193: 3-((7-(6-amino-4-methyl-3-(trifluoromethyl)pyridin-2-yl)-4-(3,8-diazabicyclo[3.2.1]octan-3-yl)-6-chloro-8-fluoroquinazolin-2-yl)oxy)-2,2-dimethylpropan-1-ol

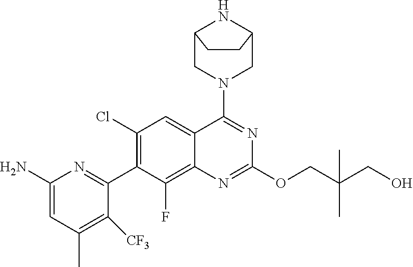 [SMILES: Cc1cc(N)nc(-c2c(Cl)cc3c(N4CC5CCC(C4)N5)nc(OCC(C)(C)CO)nc3c2F)c1C(F)(F)F]

Example 193 was prepared by similar procedure as described in Example 198 by replacing cyclopentane-1,1-diyldimethanol with 2,2-dimethylpropane-1,3-diol to give the title product (8.4 mg). ¹H NMR (400 MHz, CD₃OD) δ 7.83 (d, J=1.7 Hz, 1H), 6.60 (s, 1H), 4.61-4.41 (m, 3H), 4.23 (s, 2H), 3.71-3.60 (m, 3H), 3.44 (s, 2H), 2.44 (d, J=2.4 Hz, 3H), 1.93-1.84 (m, 4H), 1.02 (s, 6H). MS (ESI, m/e) [M+H]⁺ 569.20.

### Example 194: 3-((7-(6-amino-4-methyl-3-(trifluoromethyl)pyridin-2-yl)-4-(3,8-diazabicyclo[3.2.1]octan-3-yl)-6-chloro-8-fluoroquinazolin-2-yl)oxy)-2,2-dimethylpropanenitrile

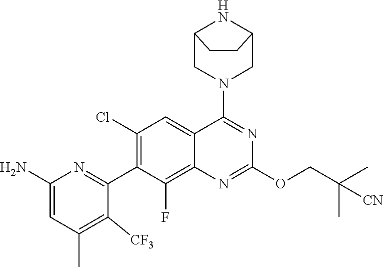 [SMILES: Cc1cc(N)nc(-c2c(Cl)cc3c(N4CC5CCC(C4)N5)nc(OCC(C)(C)C#N)nc3c2F)c1C(F)(F)F]

Example 194 was prepared by similar procedure as described in Example 206 by replacing 3-(hydroxymethyl)tetrahydrofuran-3-carbonitrile with 3-hydroxy-2,2-dimethyl propanenitrile to give the title product (11.9 mg). ¹H NMR (400 MHz, DMSO-d₆) δ 7.85 (d, J=1.6 Hz, 1H), 6.86 (s, 2H), 6.49 (s, 1H), 4.38-3.31 (m, 3H), 4.20 (d, J=12.0 Hz, 1H), 3.61-3.42 (m, 4H), 2.37 (s, 3H), 1.90 (s, 1H), 1.71-1.54 (m, 4H), 1.43 (s, 6H). MS (ESI, m/e) [M+H]⁺ 564.20.

### Example 195: 4-(((7-(6-amino-4-methyl-3-(trifluoromethyl)pyridin-2-yl)-4-(3,8-diazabicyclo[3.2.1]octan-3-yl)-6-chloro-8-fluoroquinazolin-2-yl)oxy)methyl)tetrahydro-2H-pyran-4-carbonitrile

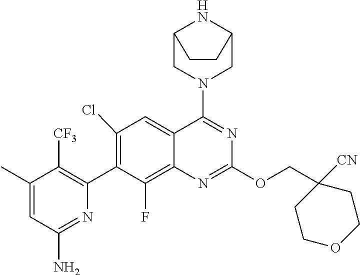 [SMILES: Cc1cc(N)nc(-c2c(Cl)cc3c(N4CC5CCC(C4)N5)nc(OCC4(C#N)CCOCC4)nc3c2F)c1C(F)(F)F]

Example 195 was prepared by similar procedure as described in Example 198 by replacing cyclopentane-1,1-diyldimethanol with 4-(hydroxymethyl) tetrahydro-2H-pyran-4-carbonitrile to give the title product (7.3 mg). ¹H NMR (400 MHz, CD₃OD) δ 7.86 (d, J=1.8 Hz, 1H), 6.60 (s, 1H), 4.52-4.43 (m, 4H), 4.09-3.92 (m, 2H), 3.81-3.59 (m, 6H), 2.51-2.41 (m, 3H), 2.14-2.01 (m, 2H), 1.94-1.77 (m, 6H). MS (ESI, m/e) [M+H]⁺ 606.25.

### Example 196: (4-(((7-(6-amino-4-methyl-3-(trifluoromethyl)pyridin-2-yl)-4-(3,8-diazabicyclo[3.2.1]octan-3-yl)-6-chloro-8-fluoroquinazolin-2-yl)oxy)methyl)tetrahydro-2H-pyran-4-yl)methanol

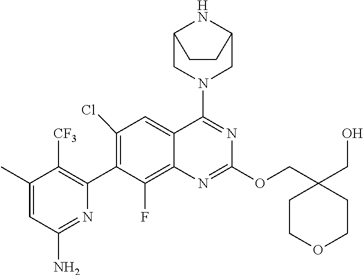 [SMILES: Cc1cc(N)nc(-c2c(Cl)cc3c(N4CC5CCC(C4)N5)nc(OCC4(CO)CCOCC4)nc3c2F)c1C(F)(F)F]

Example 196 was prepared by similar procedure as described in Example 198 by replacing cyclopentane-1,1-diyldimethanol with (tetrahydro-2H-pyran-4,4-diyl) dimethanol to give the title product (8.9 mg). 1H NMR (400 MHz, CD₃OD) δ 7.83 (d, J=1.7 Hz, 1H), 6.60 (s, 1H), 4.58-4.35 (m, 4H), 3.73 (t, J=5.7 Hz, 4H), 3.67-3.59 (m, 6H), 2.44 (s, 3H), 1.89-1.80 (m, 4H), 1.70-1.58 (m, 4H). MS (ESI, m/e) [M+H]⁺ 611.25.

### Example 197: (1-(((7-(6-amino-4-methyl-3-(trifluoromethyl)pyridin-2-yl)-4-(3,8-diazabicyclo[3.2.1]octan-3-yl)-6-chloro-8-fluoroquinazolin-2-yl)oxy)methyl)cyclopropyl) methanol

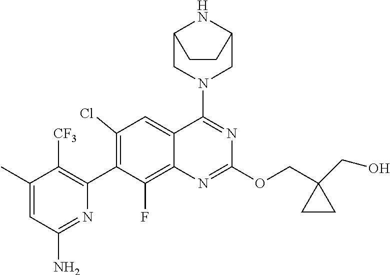 [SMILES: Cc1cc(N)nc(-c2c(Cl)cc3c(N4CC5CCC(C4)N5)nc(OCC4(CO)CC4)nc3c2F)c1C(F)(F)F]

Example 197 was prepared by similar procedure as described in Example 206 by replacing cyclopentane-1,1-diyldimethanol with cyclopropane-1,1-diyldimethanol to give the title product (1.9 mg). ¹H NMR (400 MHz, DMSO-d₆) δ 7.80 (d, J=1.6 Hz, 1H), 6.85 (s, 2H), 6.48 (s, 1H), 4.62 (t, J=5.7 Hz, 1H), 4.34-4.13 (m, 4H), 3.57-3.39 (m, 4H), 3.37 (m, 2H), 2.37 (s, 3H), 1.62 (m, 4H), 0.57-0.45 (m, 4H). MS (ESI, m/e) [M+H]⁺ 567.10.

### Example 198: (1-(((7-(6-amino-4-methyl-3-(trifluoromethyl)pyridin-2-yl)-4-(3,8-diazabicyclo[3.2.1]octan-3-yl)-6-chloro-8-fluoroquinazolin-2-yl)oxy)methyl)cyclopentyl) methanol

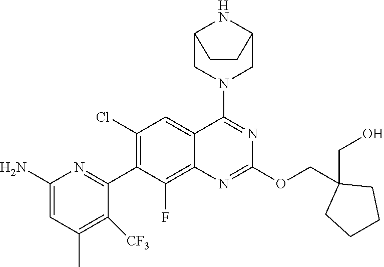 [SMILES: Cc1cc(N)nc(-c2c(Cl)cc3c(N4CC5CCC(C4)N5)nc(OCC4(CO)CCCC4)nc3c2F)c1C(F)(F)F]

Example 198 was prepared by similar procedure as described in Example 206 by replacing 3-(hydroxymethyl)tetrahydrofuran-3-carbonitrile with cyclopentane-1,1-diyldi methanol to give the title product (8.4 mg, 17% yield). ¹H NMR (400 MHz, CDCl₃) δ 7.66 (d, J=1.7 Hz, 1H), 6.46 (s, 1H), 4.80 (s, 2H), 4.55 (d, J=11.4 Hz, 1H), 4.45 (t, J=14.1 Hz, 2H), 4.30 (d, J=11.4 Hz, 1H), 3.82 (s, 2H), 3.73 (t, J=12.0 Hz, 2H), 3.49 (s, 1H), 3.35 (t, J=8.9 Hz, 2H), 2.47 (d, J=2.2 Hz, 3H), 2.05-1.85 (m, 4H), 1.75-1.55 (m, 5H), 1.55-1.40 (m, 3H). MS (ESI, m/e) [M+H]⁺ 595.25.

### Example 199: 1-(((7-(6-amino-4-methyl-3-(trifluoromethyl)pyridin-2-yl)-4-(3,8-diazabicyclo[3.2.1]octan-3-yl)-6-chloro-8-fluoroquinazolin-2-yl)oxy)methyl)cyclopentane-1-carbonitrile

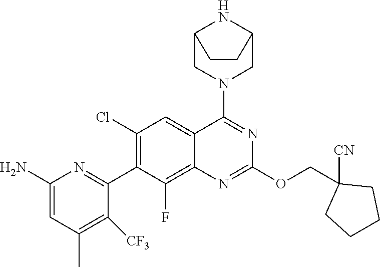 [SMILES: Cc1cc(N)nc(-c2c(Cl)cc3c(N4CC5CCC(C4)N5)nc(OCC4(C#N)CCCC4)nc3c2F)c1C(F)(F)F]

Example 199 was prepared by similar procedure as described in Example 206 by replacing 3-(hydroxymethyl)tetrahydrofuran-3-carbonitrile with 1-(hydroxymethyl)cyclopentane-1-carbonitrile to give the title product (9.8 mg). ¹H NMR (400 MHz, CD₃OD) δ 7.85 (s, 1H), 6.62 (s, 1H), 4.48 (m, 4H), 3.61 (m, 4H), 2.45 (s, 3H), 2.25-2.14 (m, 2H), 2.00 (m, 2H), 1.84 (m, 8H). MS (ESI, m/e) [M+H]⁺ 590.10.

### Example 200: (1-(((7-(6-amino-4-methyl-3-(trifluoromethyl)pyridin-2-yl)-4-(3,8-diazabicyclo[3.2.1]octan-3-yl)-6-chloro-8-fluoroquinazolin-2-yl)oxy)methyl)cyclobutyl) methanol

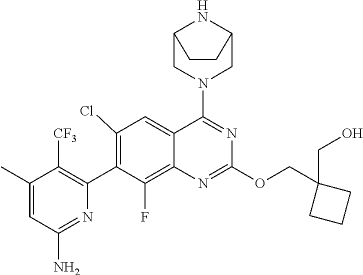 [SMILES: Cc1cc(N)nc(-c2c(Cl)cc3c(N4CC5CCC(C4)N5)nc(OCC4(CO)CCC4)nc3c2F)c1C(F)(F)F]

Example 200 was prepared by similar procedure as described in Example 198 by replacing cyclopentane-1,1-diyldimethanol with cyclobutane-1,1-diyldimethanol to give the title product (2.1 mg). ¹H NMR (400 MHz, CD₃OD) δ 7.83 (d, J=1.8 Hz, 1H), 6.73-6.49 (m, 1H), 4.46 (d, J=3.4 Hz, 4H), 4.01-3.55 (m, 6H), 2.45-2.41 (m, 3H), 2.20-1.52 (m, 10H). MS (ESI, m/e) [M+H]⁺ 581.25.

### Example 201: 6-(4-(3,8-diazabicyclo[3.2.1]octan-3-yl)-6-chloro-8-fluoro-2-((3-methyltetrahydro-2H-pyran-3-yl)methoxy)quinazolin-7-yl)-4-methyl-5-(trifluoromethyl)pyridin-2-amine

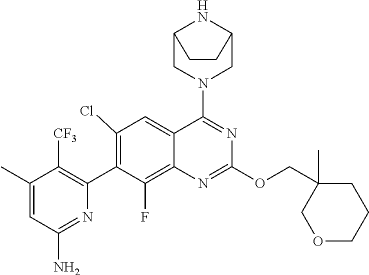 [SMILES: Cc1cc(N)nc(-c2c(Cl)cc3c(N4CC5CCC(C4)N5)nc(OCC4(C)CCCOC4)nc3c2F)c1C(F)(F)F]

Example 201 was prepared by similar procedure as described in Example 206 by replacing 3-(hydroxymethyl)tetrahydrofuran-3-carbonitrile with (3-methyltetrahydro-2H-pyran-3-yl)methanol to give the title product (14.7 mg). ¹H NMR (400 MHz, CD₃OD) δ 7.83 (s, 1H), 6.62 (s, 1H), 4.45 (m, 2H), 4.39-4.28 (m, 2H), 3.76-3.60 (m, 7H), 3.39 (d, J=11.3 Hz, 1H), 2.47 (s, 3H), 1.85 (m, 5H), 1.78-1.60 (m, 2H), 1.55-1.45 (m, 1H), 1.06 (s, 3H). MS (ESI, m/e) [M+H]⁺ 595.15.

### Example 202: 6-(4-(3,8-diazabicyclo[3.2.1]octan-3-yl)-6-chloro-8-fluoro-2-((4-methyltetrahydro-2H-pyran-4-yl)methoxy)quinazolin-7-yl)-4-methyl-5-(trifluoromethyl)pyridin-2-amine

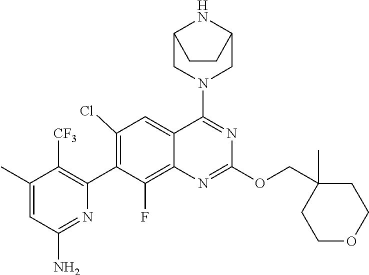 [SMILES: Cc1cc(N)nc(-c2c(Cl)cc3c(N4CC5CCC(C4)N5)nc(OCC4(C)CCOCC4)nc3c2F)c1C(F)(F)F]

Example 202 was prepared by similar procedure as described in Example 198 by replacing cyclopentane-1,1-diyldimethanol with (4-methyltetrahydro-2H-pyran-4-yl)methanol to give the title product (15.9 mg). ¹H NMR (300 MHz, CD₃OD) δ 7.85 (d, J=1.8 Hz, 1H), 6.61 (s, 1H), 4.59 (d, J=13.9 Hz, 2H), 4.27 (s, 2H), 4.21 (s, 2H), 3.91-3.59 (m, 6H), 2.45 (s, 3H), 2.15 (s, 4H), 1.80-1.74 (m, 2H), 1.49-1.40 (m, 2H), 1.19 (s, 3H). MS (ESI, m/e) [M+H]⁺ 595.35.

### Example 203: 6-(4-(3,8-diazabicyclo[3.2.1]octan-3-yl)-6-chloro-8-fluoro-2-((3-methyltetrahydrofuran-3-yl)methoxy)quinazolin-7-yl)-4-methyl-5-(trifluoromethyl)pyridin-2-amine

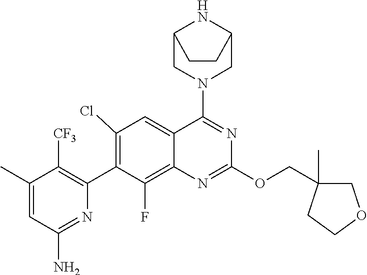 [SMILES: Cc1cc(N)nc(-c2c(Cl)cc3c(N4CC5CCC(C4)N5)nc(OCC4(C)CCOC4)nc3c2F)c1C(F)(F)F]

Example 203 was prepared by similar procedure as described in Example 206 by replacing 3-(hydroxymethyl)tetrahydrofuran-3-carbonitrile with (3-methyltetrahydrofuran-3-yl)methanol to give the title product (10.3 mg). 1H NMR (300 MHz, CD₃OD) δ 7.83 (s, 1H), 6.60 (s, 1H), 4.49-4.42 (m, 2H), 4.36-4.28 (m, 2H), 3.98-3.81 (m, 3H), 3.79-3.38 (m, 5H), 2.44 (s, 3H), 2.08-1.99 (m, 1H), 1.89-1.66 (m, 5H), 1.27 (s, 3H). MS (ESI, m/e) [M+H]⁺ 581.25.

### Example 204: 3-((7-(6-amino-4-methyl-3-(trifluoromethyl)pyridin-2-yl)-4-(3,8-diazabicyclo [3.2.1]octan-3-yl)-6-chloro-8-fluoroquinazolin-2-yl)oxy)-2-(hydroxymethyl)-2-(methoxymethyl)propanenitrile

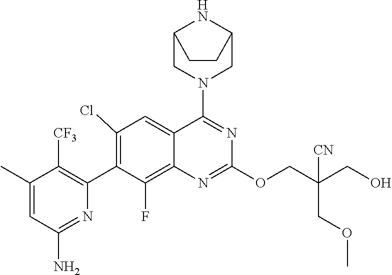 [SMILES: COCC(C#N)(CO)COc1nc(N2CC3CCC(C2)N3)c2cc(Cl)c(-c3nc(N)cc(C)c3C(F)(F)F)c(F)c2n1]

Synthetic Route

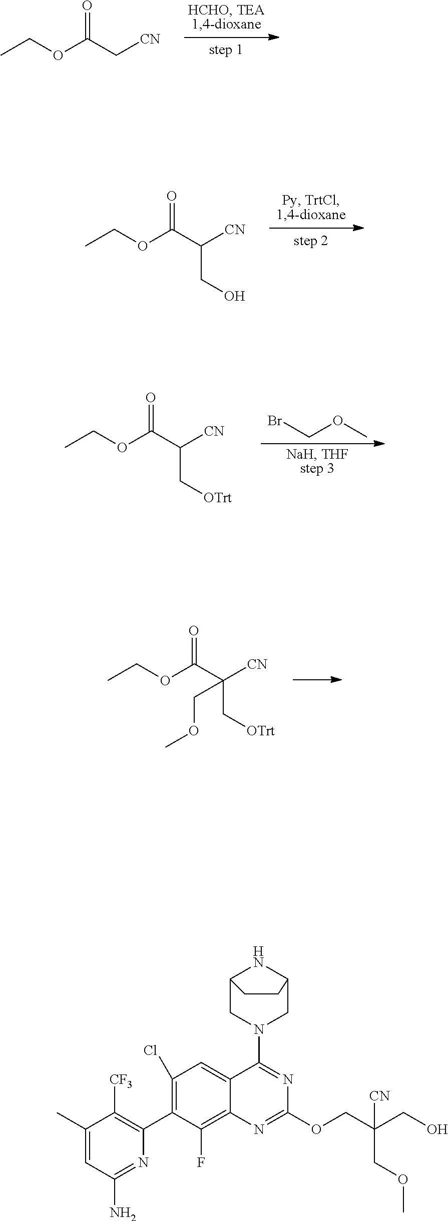

### Step 1: ethyl 2-cyano-3-hydroxypropanoate

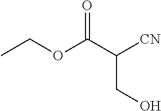

To a stirred solution of ethyl cyanoacetate (8 g, 70.72 mmol) in 1,4-dioxane (80 mL) was added HCHO (11.68 g, 155.59 mmol, 40%) and TEA (0.20 mL, 1.41 mmol) dropwise at 0° C. under nitrogen atmosphere. The resulting mixture was stirred for 6 h at rt under nitrogen atmosphere. The resulting mixture was concentrated under reduced pressure. The residue was purified by combi-flash (CH₂Cl₂/EA=1:1) to give the title product (7 g, 69% yield). MS (ESI, m/e) [M−H]⁻ 142.06.

### Step 2: ethyl 2-cyano-3-(trityloxy)propanoate

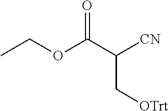

To a stirred solution of ethyl 2-cyano-3-hydroxypropanoate (3 g, 20.95 mmol) and Trt-Cl (8.76 g, 31.43 mmol) in 1,4-dioxane (60 mL) was added Pyridine (3.32 g, 41.91 mmol) dropwise at 0° C. under nitrogen atmosphere. The resulting mixture was stirred for overnight at rt under nitrogen atmosphere. The resulting mixture was concentrated under reduced pressure. The residue was purified by Combi-flash (PE/EA=3:1) to give the title product (2.4 g, 29% yield). MS (ESI, m/e) [M−H]⁻ 384.15.

### Step 3: ethyl 2-cyano-3-methoxy-2-((trityloxy)methyl)propanoate

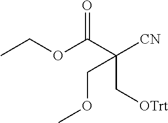

To a solution of ethyl 2-cyano-3-(triphenylmethoxy)propanoate (130 mg, 0.33 mmol) and methane, bromomethoxy- (84.29 mg, 0.67 mmol) in THF (2 mL) was added sodium hydride (60% in oil, 26.98 mg, 0.67 mmol ) at 0° C. The resulting mixture was stirred for 2 h at rt under nitrogen atmosphere. The reaction was quenched by the addition of water/ice at 0° C. The resulting mixture was concentrated under reduced pressure. The residue was purified by Prep-TLC (PE/EA=10:1) to give the title product (50 mg, 34% yield).

Example 204 was prepared by similar procedure as described in Example 205 by replacing ethyl 2-cyano-3-hydroxy-2-((trityloxy)methyl)propanoate with ethyl 2-cyano-3-methoxy-2-((trityloxy)methyl)propanoate to give the title product (4.5 mg, TFA salt). ¹H NMR (300 MHz, CD₃OD) δ 7.90 (d, J=1.8 Hz, 1H), 6.68 (s, 1H), 4.71-4.61 (m, 4H), 4.23 (m, 2H), 3.90-3.80 (m, 4H), 3.70 (m, 2H), 3.43 (s, 3H), 2.47 (s, 3H), 2.21-2.07 (m, 4H). MS (ESI, m/e) [M+H]⁺ 610.10.

### Example 205: 3-((7-(6-amino-4-methyl-3-(trifluoromethyl)pyridin-2-yl)-4-(3,8-diazabicyclo [3.2.1]octan-3-yl)-6-chloro-8-fluoroquinazolin-2-yl)oxy)-2,2-bis(hydroxymethyl) propanenitrile

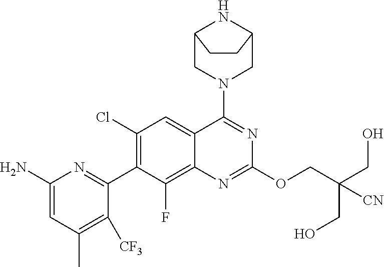 [SMILES: Cc1cc(N)nc(-c2c(Cl)cc3c(N4CC5CCC(C4)N5)nc(OCC(C#N)(CO)CO)nc3c2F)c1C(F)(F)F]

Synthetic Route

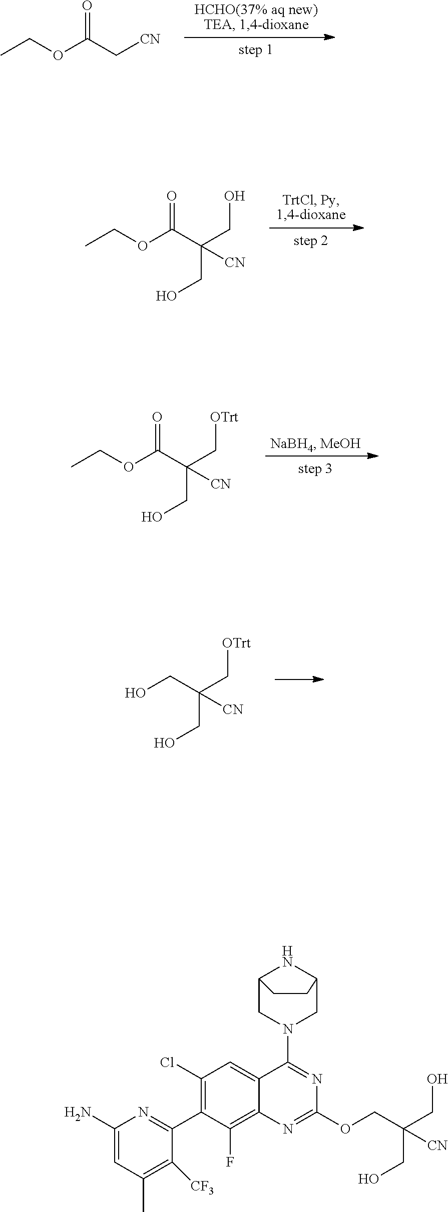

### Step 1: ethyl 2-cyano-3-hydroxy-2-(hydroxymethyl)propanoate

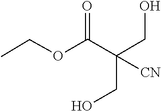

To a stirred solution of ethyl cyanoacetate (8 g, 70.72 mmol) in 1,4-dioxane (80 mL) was added HCHO (11.68 g, 155.5 mmol, 37% new) and TEA (0.20 mL, 1.41 mmol) dropwise at 0° C. under nitrogen atmosphere. The resulting mixture was stirred for 6 h at rt under. The resulting mixture was concentrated under reduced pressure. The crude product was used in the next step without purification.

### Step 2: ethyl 2-cyano-3-hydroxy-2-((trityloxy)methyl)propanoate

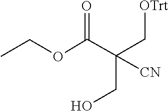

To a stirred solution of ethyl 2-cyano-3-hydroxy-2-(hydroxymethyl)propanoate (8 g, 46.19 mmol) and Trt-Cl (25.76 g, 92.39 mmol) in 1,4-dioxane (80 mL) was added Pyridine (7.31 g, 92.39 mmol) dropwise at 0° C. The resulting mixture was stirred overnight at rt. The resulting mixture was concentrated under reduced pressure. The residue was purified by combi-flash (PE/EA=5:1) to give the title product (215 mg, 1% yield).

### Step 3: 3-hydroxy-2-(hydroxymethyl)-2-((trityloxy)methyl)propanenitrile

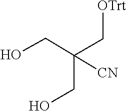

To a stirred solution of ethyl 2-cyano-3-hydroxy-2-((triphenylmethoxy)methyl) propanoate (215 mg, 0.51 mmol) in MeOH (2 mL) was added NaBH₄ (39.15 mg, 1.03 mmol) in portions at 0° C. under nitrogen atmosphere. The resulting mixture was stirred for 2 h at rt under nitrogen atmosphere. The reaction was quenched with 1 M HCl (aq) at 0° C. The resulting mixture was extracted with EtOAc. The combined organic layers were washed with brine and dried over anhydrous Na₂SO₄. After filtration, the filtrate was concentrated under reduced pressure. The residue was purified by Prep-TLC (PE/EA=3:1) to give the title product (25 mg, 13% yield)

Example 205 was prepared by similar procedure as described in Example 206 by replacing 3-(hydroxymethyl)tetrahydrofuran-3-carbonitrile with 3-hydroxy-2-(hydroxymethyl)-2-((trityloxy)methyl)propanenitrile to give the title product (8.4 mg, TFA salt). ¹H NMR (300 MHz, CD₃OD) δ 7.89 (d, J=1.7 Hz, 1H), 6.66 (s, 1H), 4.66 (d, J=11.6 Hz, 4H), 4.23 (s, 2H), 3.90-3.80 (m, 6H), 2.50 (s, 3H), 2.20-2.10 (m, 4H). MS (ESI, m/e) [M+H]⁺ 596.10.

### Example 206: 3-(((7-(6-amino-4-methyl-3-(trifluoromethyl)pyridin-2-yl)-4-(3,8-diazabicyclo[3.2.1]octan-3-yl)-6-chloro-8-fluoroquinazolin-2-yl)oxy)methyl)tetrahydrofuran-3-carbonitrile

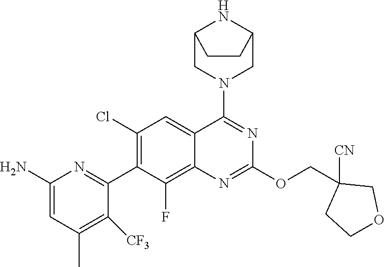 [SMILES: Cc1cc(N)nc(-c2c(Cl)cc3c(N4CC5CCC(C4)N5)nc(OCC4(C#N)CCOC4)nc3c2F)c1C(F)(F)F]

Synthetic Route

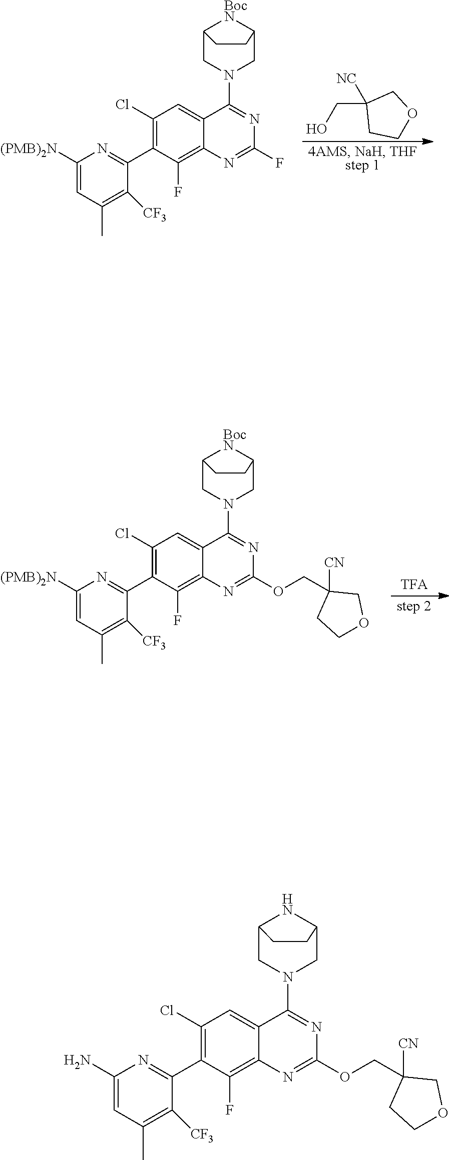

### Step 1: tert-butyl 3-(7-(6-(bis(4-methoxybenzyl)amino)-4-methyl-3-(trifluoro methyl) pyridin-2-yl)-6-chloro-2-((3-cyanotetrahydrofuran-3-yl)methoxy)-8-fluoroquinazolin-4-yl)-3,8-diazabicyclo[3.2.1]octane-8-carboxylate

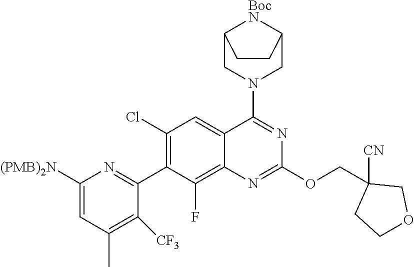

To a 25-mL round bottom flask was added tert-butyl 3-(7-(6-(bis((4-methoxy phenyl)methyl)amino)-4-methyl-3-(trifluoromethyl)pyridin-2-yl)-6-chloro-2,8-difluoro quinazolin-4-yl)-3,8-diazabicyclo[3.2.1]octane-8-carboxylate (100 mg, 0.121 mmol), 3-(hydroxymethyl)oxolane-3-carbonitrile (15.41 mg, 0.121 mmol), THF (1 mL) and molecular sieves (4A) (30 mg). NaH (7 mg, 0.181 mmol, 60%) was added at 0° C. under N₂ atmosphere. The reaction was stirred at R.T. for 1.5 h. The reaction was quenched by NH₄Cl (100 mL), extracted by EA (30 mL*3). The combined organic phase was dried by anhydrous Na₂SO₄ and concentrated under vacuum. The residue was purified by TLC (PE:EA=1:1, ref=0.3) to give the title product (92 mg, 81% yield). MS (ESI, m/e) [M+H]⁺ 932.65.

### Step 2: 3-(((7-(6-amino-4-methyl-3-(trifluoromethyl)pyridin-2-yl)-4-(3,8-diaza bicyclo[3.2.1]octan-3-yl)-6-chloro-8-fluoroquinazolin-2-yl)oxy)methyl)tetrahydrofuran-3-carbonitrile

To a 25-mL round bottom flask was added tert-butyl 3-(7-(6-(bis((4-methoxyphenyl)methyl)amino)-4-methyl-3-(trifluoromethyl)pyridin-2-yl)-6-chloro-2-((3-cyanooxolan-3-yl)methoxy)-8-fluoroquinazolin-4-yl)-3,8-diazabicyclo[3.2.1]octane-8-carboxylate (92 mg, 0.099 mmol) and TFA (3 mL). The reaction was stirred at 50° C. for 2 h. The reaction was concentrated under vacuum. The residue was solved in EA (100 mL) and saturated Na₂CO₃ aqueous solution (100 mL) was added. The solution was stirred at rt for 10 min and extracted by EA (50 mL*3). The combined organic phase was washed with brine (50 mL*3), dried by Na₂SO₄, and then concentrated under vacuum. The residue was purified by Prep-HPLC to give the title product (13.2 mg, 22% yield). ¹H NMR (400 MHz, CD₃OD) δ 7.86 (d, J=1.7 Hz, 1H), 6.62-6.57 (m, 1H), 4.64 (dd, J=10.8, 1.6 Hz, 1H), 4.49 (dd, J=33.4, 11.3 Hz, 3H), 4.09 (d, J=9.3 Hz, 1H), 4.05-3.93 (m, 3H), 3.66-3.55 (m, 4H), 2.52-2.41 (m, 4H), 2.30 (m, 1H), 1.82 (m, 4H). MS (ESI, m/e) [M+H]⁺ 592.2.

### Example 207: 5-(((7-(6-amino-4-methyl-3-(trifluoromethyl)pyridin-2-yl)-4-(3,8-diazabicyclo[3.2.1]octan-3-yl)-6-chloro-8-fluoroquinazolin-2-yl)oxy)methyl)-5-methylpyrrolidin-2-one

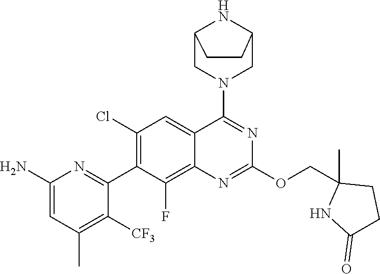 [SMILES: Cc1cc(N)nc(-c2c(Cl)cc3c(N4CC5CCC(C4)N5)nc(OCC4(C)CCC(=O)N4)nc3c2F)c1C(F)(F)F]

Example 207 was prepared by similar procedure as described in Example 206 by replacing 3-(hydroxymethyl)tetrahydrofuran-3-carbonitrile with 5-(hydroxymethyl)-5-methylpyrrolidin-2-one to give the title product (17.4 mg). ¹H NMR (300 MHz, CD₃OD) δ 7.84 (d, J=1.8 Hz, 1H), 6.60 (s, 1H), 4.55-4.27 (m, 4H), 3.66-3.55 (s, 4H), 2.65-2.51 (m, 1H), 2.50-2.17 (m, 5H), 2.07-1.70 (m, 5H), 1.40 (s, 3H). MS (ESI, m/e) [M+H]*594.10.

### Example 208: 4-(((7-(6-amino-4-methyl-3-(trifluoromethyl)pyridin-2-yl)-4-(3,8-diazabicyclo[3.2.1]octan-3-yl)-6-chloro-8-fluoroquinazolin-2-yl)oxy)methyl)-4-methylpyrrolidin-2-one

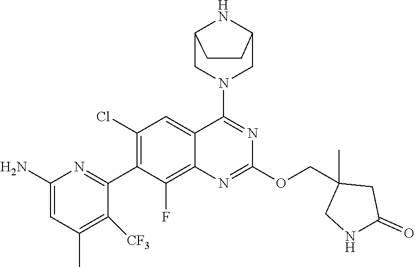 [SMILES: Cc1cc(N)nc(-c2c(Cl)cc3c(N4CC5CCC(C4)N5)nc(OCC4(C)CNC(=O)C4)nc3c2F)c1C(F)(F)F]

Example 208 was prepared by similar procedure as described in Example 206 by replacing 3-(hydroxymethyl)tetrahydrofuran-3-carbonitrile with 4-(hydroxymethyl)-4-methyl pyrrolidin-2-one to give the title product (8.2 mg). ¹H NMR (300 MHz, CD₃OD) δ 7.85 (s, 1H), 6.60 (s, 1H), 4.53 (s, 2H), 4.36 (s, 2H), 3.94 (s, 2H), 3.72-3.67 (d, J=12.8 Hz, 2H), 3.52-3.49 (d, J=10.2 Hz, 1H), 3.22-3.13 (d, J=10.2 Hz, 1H), 2.60-2.37 (m, 4H), 2.21 (s, 1H), 2.15 (s, 1H), 1.99 (s, 3H), 1.33 (s, 3H). MS (ESI, m/e) [M+H]⁺ 594.10.

### Example 209: 4-(((7-(2-amino-7-fluorobenzo[d]thiazol-4-yl)-4-(3,8-diazabicyclo [3.2.1]octan-3-yl)-8-fluoro-6-(trifluoromethyl)quinazolin-2-yl)oxy)methyl)tetrahydrofuran-3-ol

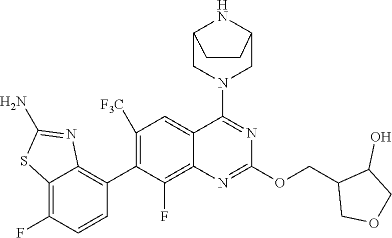 [SMILES: Nc1nc2c(-c3c(C(F)(F)F)cc4c(N5CC6CCC(C5)N6)nc(OCC5COCC5O)nc4c3F)ccc(F)c2s1]

Synthetic Route

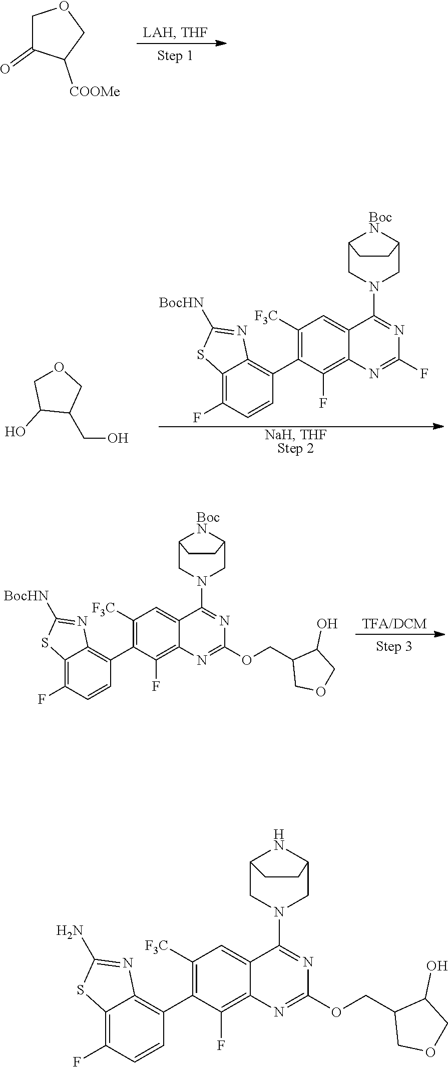

### Step 1: 4-(hydroxymethyl)tetrahydrofuran-3-ol

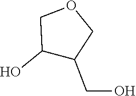

To a solution of methyl 4-oxotetrahydrofuran-3-carboxylate (288.0 mg, 2.0 mmol) in THF (15 mL) was added sodium hydride (76.0 mg, 2.0 mmol) at 0° C. The resulting mixture was stirred at room temperature for 1 hrs. After completion, the reaction mixture was quenched with Na₂SO₄·10H₂O, filtered and the filtrate was concentrated to give the title product (100 mg, crude).

### Step 2: tert-butyl 3-(7-(2-((tert-butoxycarbonyl)amino)-7-fluorobenzo[d] thiazol-4-yl)-8-fluoro-2-((4-hydroxytetrahydrofuran-3-yl)methoxy)-6-(trifluoromethyl) quinazolin-4-yl)-3,8-diazabicyclo[3.2.1]octane-8-carboxylate

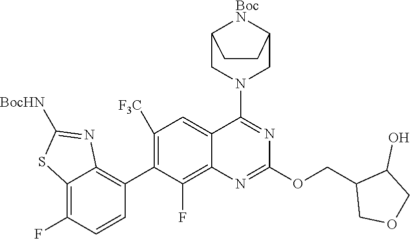 [SMILES: CC(C)(C)OC(=O)Nc1nc2c(-c3c(C(F)(F)F)cc4c(N5CC6CCC(C5)N6C(=O)OC(C)(C)C)nc(OCC5COCC5O)nc4c3F)ccc(F)c2s1]

To a solution of 4-(hydroxymethyl)tetrahydrofuran-3-ol (35.4 mg, 0.30 mmol) in THF (5 mL) was added sodium hydride (13.42 mg, 0.33 mmol) at room temperature. The resulting mixture was stirred at room temperature for 1 hrs. Then, tert-butyl 3-(7-(2-((tert-butoxycarbonyl)amino)-7-fluorobenzo[d]thiazol-4-yl)-2,8-difluoro-6-(trifluoromethyl) quinazolin-4-yl)-3,8-diazabicyclo[3.2.1]octane-8-carboxylate (71.0 mg, 0.10 mmol) was added to the reaction mixture and the mixture was stirred at room temperature for 1 hrs. After completion, the reaction mixture was diluted with EA (50 mL) and washed with saturated NaCl (15 mL×3). The organic layer was dried over anhydrous Na₂SO₄, filtered and the filtrate was concentrated to give the residue. The residue was purified by Prep-TLC (PE:EA=1:3) to give the title product (40.5 mg, 0.05 mmol). MS (ESI, m/e) [M+1]⁺809.3.

### Step 3: 4-(((7-(2-amino-7-fluorobenzo[d]thiazol-4-yl)-4-(3,8-diazabicyclo [3.2.1]octan-3-yl)-8-fluoro-6-(trifluoromethyl)quinazolin-2-yl)oxy)methyl)tetrahydrofuran-3-ol

To a solution of tert-butyl 3-(7-(2-((tert-butoxycarbonyl)amino)-7-fluoro benzo[d]thiazol-4-yl)-8-fluoro-2-((4-hydroxytetrahydrofuran-3-yl)methoxy)-6-(trifluoro methyl)quinazolin-4-yl)-3,8-diazabicyclo[3.2.1]octane-8-carboxylate (40.5 mg, 0.05 mmol) in DCM (5 mL) was added TFA (5 mL) at room temperature. The resulting mixture was stirred at room temperature for 3 hrs. After completion, the reaction mixture was concentrated to give the residue. The residue was purified by High-HPLC to give the title product (2.37 mg, FA salt). ¹H NMR (500 MHz, CD₃OD) δ 8.50 (s, 1H), 8.12 (s, 1H), 7.19-7.18 (m, 1H), 6.97-6.95 (m, 1H), 4.55-4.46 (m, 3H), 4.10-3.93 (m, 5H), 3.81-3.68 (m, 5H), 1.99-1.98 (m, 5H). MS (ESI, m/e) [M+1]⁺609.7.

### Example 210: 4-(2-((3-oxabicyclo[3.1.0]hexan-6-yl)methoxy)-4-(3,8-diaza bicyclo[3.2.1]octan-3-yl)-8-fluoro-6-(trifluoromethyl)quinazolin-7-yl)-7-fluorobenzo[d]thiazol-2-amine

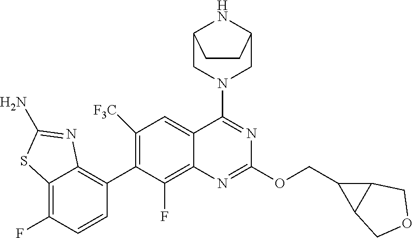 [SMILES: Nc1nc2c(-c3c(C(F)(F)F)cc4c(N5CC6CCC(C5)N6)nc(OCC5C6COCC65)nc4c3F)ccc(F)c2s1]

Example 210 was prepared by similar procedure as described in Example 145 by replacing (tetrahydrofuran-3-yl)methanol with (3-oxabicyclo[3.1.0]hexan-6-yl) methanol to give the title product (2 mg, FA salt, 13% yield). 1H NMR (400 MHz, CD₃OD) δ 8.48 (s, 1H), 8.11-8.03 (m, 1H), 7.19-7.11 (m, 1H), 6.98-6.95 (m, 1H), 4.56-4.40 (m, 3H), 4.10-3.31 (m, 9H), 2.00-1.91 (m, 5H), 1.51-0.77 (m, 2H). MS (ESI, m/e) [M+H]⁺ 605.4.

### Example 211: 3-(((7-(2-amino-7-fluorobenzo[d]thiazol-4-yl)-4-(3,8-diazabicyclo [3.2.1]octan-3-yl)-8-fluoro-6-(trifluoromethyl)quinazolin-2-yl)oxy)methyl)tetrahydrofuran-3-carbonitrile

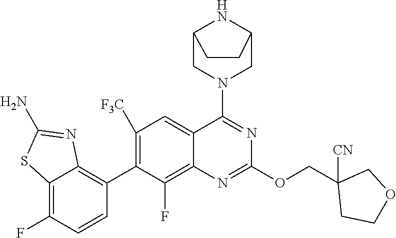 [SMILES: N#CC1(COc2nc(N3CC4CCC(C3)N4)c3cc(C(F)(F)F)c(-c4ccc(F)c5sc(N)nc45)c(F)c3n2)CCOC1]

Example 211 was prepared by similar procedure as described in Example 166 by replacing (2-methyloxetan-2-yl)methanol with 3-(hydroxymethyl)tetrahydrofuran-3-carbonitrile to give the title product (16.34 mg). ¹H NMR (500 MHz, CD₃OD) δ 8.14 (s, 1H), 7.30-7.10 (m, 1H), 6.97 (t, J=8.8 Hz, 1H), 4.79-4.63 (m, 3H), 4.60-4.56 (m, 1H), 4.24 (s, 2H), 4.13-3.90 (m, 6H), 2.47-2.44 (m, 1H), 2.32-2.28 (m, 1H), 2.12 (d, J=11.3 Hz, 4H). MS (ESI, m/e) [M+H]⁺ 618.4.

### Example 212: 5-(((7-(2-amino-7-fluorobenzo[d]thiazol-4-yl)-4-(3,8-diaza bicyclo[3.2.1]octan-3-yl)-8-fluoro-6-(trifluoromethyl)quinazolin-2-yl)oxy)methyl)-5-methylpyrrolidin-2-one

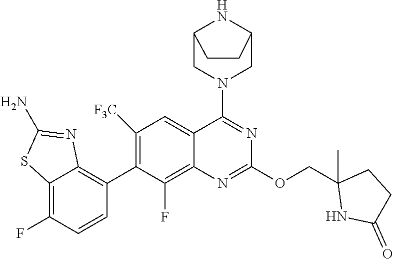 [SMILES: CC1(COc2nc(N3CC4CCC(C3)N4)c3cc(C(F)(F)F)c(-c4ccc(F)c5sc(N)nc45)c(F)c3n2)CCC(=O)N1]

Example 212 was prepared by similar procedure as described in Example 166 by replacing (2-methyloxetan-2-yl)methanol with 5-(hydroxymethyl)-5-methylpyrrolidin-2-one to give the title product (16.78 mg). ¹H NMR (500 MHz, DMSO-d₆) δ 8.08 (s, 1H), 7.90 (s, 2H), 7.77 (s, 1H), 7.27-7.13 (m, 1H), 7.04 (t, J=8.7 Hz, 1H), 4.50 (d, J=10.8 Hz, 1H), 4.39 (s, 1H), 4.23 (d, J=12.9 Hz, 2H), 4.02 (s, 2H), 3.78-3.67 (m, 2H), 2.59 (s, 2H), 2.37-2.06 (m, 3H), 1.85-1.77 (m, 4H), 1.27 (s, 3H). MS (ESI, m/e) [M+H]⁺ 620.2.

### Example 213: 2-(((7-(2-amino-7-fluorobenzo[d]thiazol-4-yl)-4-(3,8-diaza bicyclo[3.2.1]octan-3-yl)-8-fluoro-6-(trifluoromethyl)quinazolin-2-yl)oxy)methyl)cyclohexan-1-ol

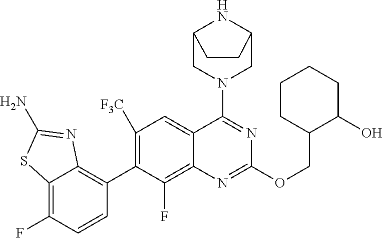 [SMILES: Nc1nc2c(-c3c(C(F)(F)F)cc4c(N5CC6CCC(C5)N6)nc(OCC5CCCCC5O)nc4c3F)ccc(F)c2s1]

Example 213 was prepared by similar procedure as described in Example 209 by replacing methyl 4-oxotetrahydrofuran-3-carboxylate with methyl 2-oxocyclo hexane-1-carboxylate to give the title product (2.29 mg). ¹H NMR (500 MHz, CD₃OD) δ 8.11 (s, 1H), 7.19-7.18 (m, 1H), 6.97-6.95 (m, 1H), 4.61-4.52 (m, 4H), 4.06-3.74 (m, 5H), 1.99-1.95 (m, 6H), 1.83-1.31 (m, 7H). MS (ESI, m/e) [M+H]⁺ 621.7.

### Example 214: 1-(((7-(2-amino-7-fluorobenzo[d]thiazol-4-yl)-4-(3,8-diazabicyclo [3.2.1]octan-3-yl)-8-fluoro-6-(trifluoromethyl)quinazolin-2-yl)oxy)methyl)cyclobutane-1-carbonitrile

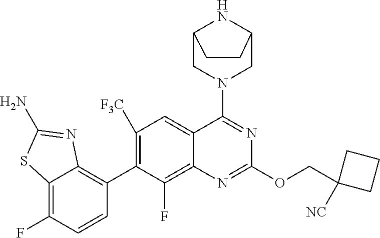 [SMILES: N#CC1(COc2nc(N3CC4CCC(C3)N4)c3cc(C(F)(F)F)c(-c4ccc(F)c5sc(N)nc45)c(F)c3n2)CCC1]

Example 214 was prepared by similar procedure as described in Step 2 and step 3 in Example 209 by replacing 4-(hydroxymethyl)tetrahydrofuran-3-ol with 1-(hydroxymethyl)cyclobutane-1-carbonitrile to give the title product (9.17 mg). ¹HNMR (500 MHz, CD₃OD) δ 8.14 (s, 1H), 7.21-7.18 (m, 1H), 6.98-6.95 (m, 1H), 4.71-4.59 (m, 4H), 3.93-3.78 (m, 4H), 2.60-2.56 (m, 2H), 2.43-2.40 (m, 2H), 2.22-2.19 (m, 2H), 2.03-1.95 (m, 4H). MS (ESI, m/e) [M+H]⁺ 602.6.

### Example 215: 4-(4-(3,8-diazabicyclo[3.2.1]octan-3-yl)-8-fluoro-2-((2-methyl-1,4-dioxan-2-yl)methoxy)-6-(trifluoromethyl)quinazolin-7-yl)-7-fluorobenzo[d]thiazol-2-amine

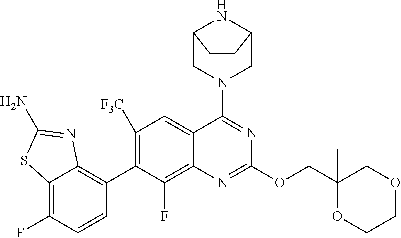 [SMILES: CC1(COc2nc(N3CC4CCC(C3)N4)c3cc(C(F)(F)F)c(-c4ccc(F)c5sc(N)nc45)c(F)c3n2)COCCO1]

Example 215 was prepared by similar procedure as described in Example 166 by replacing (2-methyloxetan-2-yl)methanol with (2-methyl-1,4-dioxan-2-yl)methanol to give the title product (14.23 mg). ¹H NMR (500 MHz, CD₃OD) δ 8.12 (s, 1H), 7.32-7.06 (m, 1H), 6.96 (t, J=8.7 Hz, 1H), 4.74-4.57 (m, 3H), 4.52-4.49 (m, 1H), 4.06 (s, 2H), 3.78-3.67 (m, 7H), 3.52 (d, J=11.6 Hz, 1H), 2.03 (s, 4H), 1.32 (s, 3H). MS (ESI, m/e) [M+H]⁺ 623.3.

### Example 216: 4-(4-(3,8-diazabicyclo[3.2.1]octan-3-yl)-8-fluoro-2-((tetrahydro-2H-pyran-3-yl)methoxy)-6-(trifluoromethyl)quinazolin-7-yl)-7-fluorobenzo[d]thiazol-2-amine

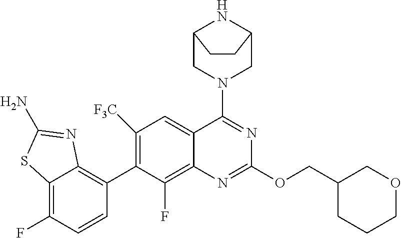 [SMILES: Nc1nc2c(-c3c(C(F)(F)F)cc4c(N5CC6CCC(C5)N6)nc(OCC5CCCOC5)nc4c3F)ccc(F)c2s1]

Example 216 was prepared by similar procedure as described in Example 166 by replacing (2-methyloxetan-2-yl)methanol with (tetrahydro-2H-pyran-3-yl)methanol to give the title product (16.73 mg). ¹H NMR (500 MHz, DMSO-d₆) δ 8.07 (s, 1H), 7.90 (s, 2H), 7.18 (d, J=5.8 Hz, 1H), 7.03 (t, J=8.7 Hz, 1H), 4.40 (d, J=12.5 Hz, 1H), 4.35-4.12 (m, 3H), 3.86 (d, J=10.2 Hz, 1H), 3.79-3.62 (m, 4H), 3.56 (d, J=11.9 Hz, 1H), 3.36 (d, J=11.0 Hz, 2H), 3.26 (d, J=10.0 Hz, 1H), 2.03 (s, 1H), 1.82 (s, 1H), 1.78-1.34 (m, 7H). MS (ESI, m/e) [M+H]⁺ 607.5.

### Example 217: 4-(2-((3-oxabicyclo[3.1.0]hexan-1-yl)methoxy)-4-(3,8-diaza bicyclo[3.2.1]octan-3-yl)-8-fluoro-6-(trifluoromethyl)quinazolin-7-yl)-7-fluorobenzo[d]thiazol-2-amine

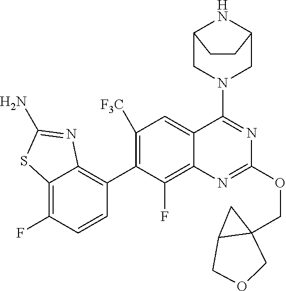 [SMILES: Nc1nc2c(-c3c(C(F)(F)F)cc4c(N5CC6CCC(C5)N6)nc(OCC56COCC5C6)nc4c3F)ccc(F)c2s1]

Example 217 was prepared by similar procedure as described in Example 166 by replacing (2-methyloxetan-2-yl)methanol with (3-oxabicyclo[3.1.0]hexan-1-yl)methanol to give the title product (6.33 mg). ¹H NMR (500 MHz, DMSO-d₆) δ 8.07 (s, 1H), 7.89 (s, 2H), 7.17 (s, 1H), 7.04 (t, J=8.8 Hz, 1H), 4.76-4.62 (m, 1H), 4.51-4.43 (m, 4H), 4.14 (s, 2H), 3.81 (d, J=7.7 Hz, 2H), 3.74-3.63 (m, 3H), 2.54 (s, 1H), 1.90 (s, 4H), 1.70 (s, 1H), 0.87 (d, J=4.0 Hz, 1H), 0.59 (s, 1H). MS (ESI, m/e) [M+H]⁺ 605.5.

### Example 218: 4-(4-(3,8-diazabicyclo[3.2.1]octan-3-yl)-8-fluoro-2-((4-fluorotetra hydro-2H-pyran-4-yl)methoxy)-6-(trifluoromethyl)quinazolin-7-yl)-7-fluorobenzo[d]thiazol-2-amine

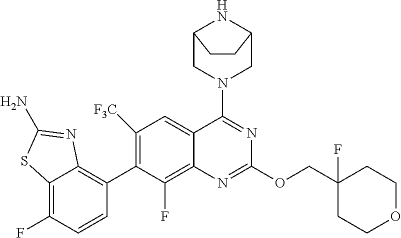 [SMILES: Nc1nc2c(-c3c(C(F)(F)F)cc4c(N5CC6CCC(C5)N6)nc(OCC5(F)CCOCC5)nc4c3F)ccc(F)c2s1]

Example 218 was prepared by similar procedure as described in Example 166 by replacing (2-methyloxetan-2-yl)methanol with (1-fluorocyclohexyl)methanol to give the title product (14.93 mg). ¹H NMR (500 MHz, CD₃OD) δ 8.13 (s, 1H), 7.23-7.12 (m, 1H), 6.97 (t, J=8.8 Hz, 1H), 4.67 (d, J=11.8 Hz, 2H), 4.56 (d, J=20.1 Hz, 2H), 4.11 (s, 2H), 3.97-3.79 (m, 4H), 3.77-3.73 (m, 2H), 2.16-1.81 (m, 8H). MS (ESI, m/e) [M+H]⁺ 625.4.

### Example 219: 4-(2-(((S)-1,4-dioxan-2-yl)methoxy)-4-(3,8-diazabicyclo[3.2.1] octan-3-yl)-8-fluoro-6-(trifluoromethyl)quinazolin-7-yl)-7-fluorobenzo[d]thiazol-2-amine

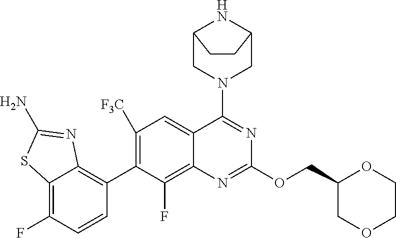 [SMILES: Nc1nc2c(-c3c(C(F)(F)F)cc4c(N5CC6CCC(C5)N6)nc(OC[C@@H]5COCCO5)nc4c3F)ccc(F)c2s1]

Example 219 was prepared by similar procedure as described in Example 166 by replacing (2-methyloxetan-2-yl)methanol with (S)-(1,4-dioxan-2-yl)methanol to give the title product (16.3 mg). ¹H NMR (500 MHz, CD₃OD) δ 8.12 (s, 1H), 7.33-7.11 (m, 1H), 6.96 (t, J=8.8 Hz, 1H), 4.69-4.52 (m, 2H), 4.46-4.41 (m, 2H), 3.97-3.88 (m, 4H), 3.85-3.68 (m, 5H), 3.67-3.49 (m, 2H), 1.97 (s, 4H). MS (ESI, m/e) [M+H]⁺ 609.4.

### Example 220: 2-(((7-(2-amino-7-fluorobenzo[d]thiazol-4-yl)-4-(3,8-diazabicyclo [3.2.1]octan-3-yl)-8-fluoro-6-trifluoromethyl)quinazolin-2-yl)oxy)methyl)cyclopentan-1-ol

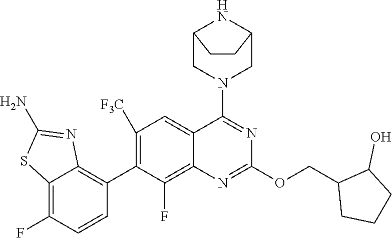 [SMILES: Nc1nc2c(-c3c(C(F)(F)F)cc4c(N5CC6CCC(C5)N6)nc(OCC5CCCC5O)nc4c3F)ccc(F)c2s1]

Example 220 was prepared by similar procedure as described in Example 145 by replacing (tetrahydrofuran-3-yl)methanol with 2-(hydroxymethyl)cyclopentan-1-ol to give the title product (4 mg, 16% yield). ¹H NMR (500 MHz, CD₃OD) δ 8.25 (s, 1H), 7.25 (s, 1H), 7.04 (t, J=8.7 Hz, 1H), 5.72 (d, J=123.2 Hz, 1H), 5.16-4.90 (m, 2H), 4.35-4.28 (m, 2H), 4.18-4.03 (m, 2H), 3.87-3.42 (m, 2H), 2.54-2.30 (m, 1H), 2.28-1.55 (m, 10H). MS (ESI, m/e) [M+H]⁺ 607.4.

### Example 221: 4-(((7-(2-amino-7-fluorobenzo[d]thiazol-4-yl)-4-(3,8-diazabicyclo [3.2.1]octan-3-yl)-8-fluoro-6-(trifluoromethyl)quinazolin-2-yl)oxy)methyl)tetrahydro-2H-pyran-4-ol

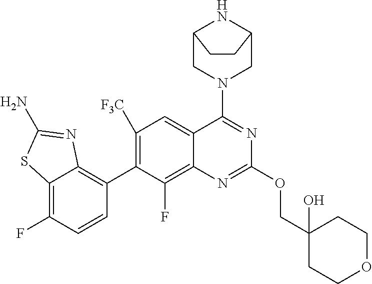 [SMILES: Nc1nc2c(-c3c(C(F)(F)F)cc4c(N5CC6CCC(C5)N6)nc(OCC5(O)CCOCC5)nc4c3F)ccc(F)c2s1]

Example 221 was prepared by similar procedure as described in Example 209 by replacing methyl 4-oxotetrahydrofuran-3-carboxylate with 4-hydroxytetrahydro-2H-pyran-4-carboxylic acid to give the title product (2.54 mg). ¹HNMR (500 MHz, CD₃OD) δ 8.12 (s, 1H), 7.19-7.18 (m, 1H), 6.98-6.95 (m, 1H), 4.56-4.55 (m, 2H), 4.33 (s, 2H), 3.85-3.73 (m, 8H), 1.93-1.84 (m, 6H), 1.65-1.62 (m, 2H). MS (ESI, m/e) [M+H]⁺ 623.4.

### Example 222: 3-(((7-(2-amino-7-fluorobenzo[d]thiazol-4-yl)-4-(3,8-diazabicyclo [3.2.1]octan-3-yl)-8-fluoro-6-(trifluoromethyl)quinazolin-2-yl)oxy)methyl)bicyclo[1.1.1]pentan-1-ol

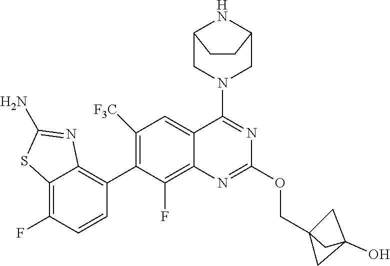 [SMILES: Nc1nc2c(-c3c(C(F)(F)F)cc4c(N5CC6CCC(C5)N6)nc(OCC56CC(O)(C5)C6)nc4c3F)ccc(F)c2s1]

Example 222 was prepared by similar procedure as described in Example 209 by replacing methyl 4-oxotetrahydrofuran-3-carboxylate with 1,3-hydroxybicyclo [1.1.1]pentane-1-carboxylic acid to give the title product (2.29 mg). ¹H NMR (500 MHz, CD₃OD) δ 8.12 (s, 1H), 7.19-7.18 (m, 1H), 7.00-6.95 (m, 1H), 4.71-4.63 (m, 4H), 4.24 (s, 2H), 3.90-3.87 (m, 2H), 2.15-2.14 (m, 4H), 1.91-1.89 (m, 6H). MS (ESI, m/e) [M+H]⁺ 605.4.

### Example 223: 1-(3-(((7-(2-amino-7-fluorobenzo[d]thiazol-4-yl)-4-(3,8-diaza bicyclo[3.2.1]octan-3-yl)-8-fluoro-6-(trifluoromethyl)quinazolin-2-yl)oxy)methyl) piperidin-1-yl)ethan-1-one

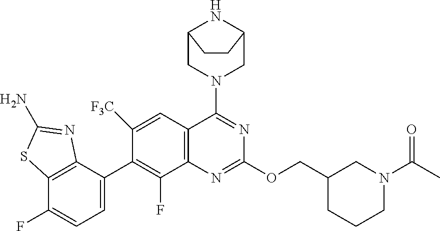 [SMILES: CC(=O)N1CCCC(COc2nc(N3CC4CCC(C3)N4)c3cc(C(F)(F)F)c(-c4ccc(F)c5sc(N)nc45)c(F)c3n2)C1]

Synthetic Route

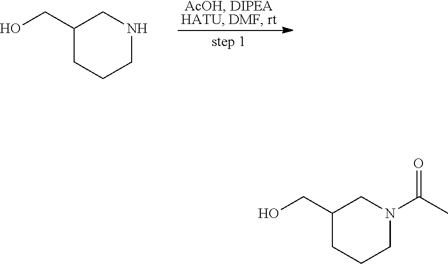

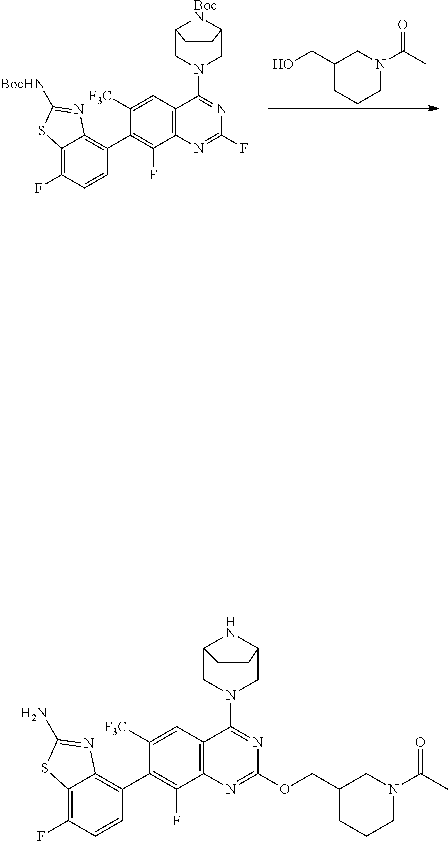 [SMILES: CC(C)(C)OC(=O)Nc1nc2c(-c3c(C(F)(F)F)cc4c(N5CC6CCC(C5)N6C(=O)OC(C)(C)C)nc(F)nc4c3F)ccc(F)c2s1 ; CC(=O)N1CCCC(CO)C1 ; CC(=O)N1CCCC(COc2nc(N3CC4CCC(C3)N4)c3cc(C(F)(F)F)c(-c4ccc(F)c5sc(N)nc45)c(F)c3n2)C1]

### Step 1:1-(3-(hydroxymethyl)piperidin-1-yl)ethan-1-one

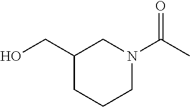

To a 25-mL round bottom flask was added piperidin-3-ylmethanol (460 mg, 4.0 mmol), acetic acid (240 mg, 4.0 mmol), HATU (1.5 g, 4.0 mmol), N,N-diisopropyl ethylamine (1.6 g, 4.0 mmol) and DMF (20 mL). The reaction was stirred at rt for 16 h. The reaction was quenched by brine (20 mL), extracted by EA (30 mL*2) and washed by brine (20 mL*3). The combined organic phase was dried by anhydrous Na₂SO₄ and concentrated under vacuum to give the crude title product (300 mg, 64.6% yield). MS (ESI, m/e) [M+H]⁺ 158.5.

Example 223 was prepared by similar procedure as described in Example 145 by replacing (tetrahydrofuran-3-yl)methanol with 1-(3-(hydroxymethyl)piperidin-1-yl)ethan-1-one to give the title product (2.5 mg). ¹H NMR (500 MHz, CD₃OD) δ 8.11 (d, J=4.3 Hz, 1H), 7.24-7.13 (m, 1H), 6.97 (t, J=8.7 Hz, 1H), 4.64 (t, J=11.5 Hz, 2H), 4.55-4.41 (m, 1H), 4.41-4.27 (m, 1H), 4.22-3.91 (m, 3H), 3.88-3.75 (m, 2H), 3.18 (dd, J=23.9, 12.2 Hz, 1H), 2.14-1.92 (m, 8H), 1.87-1.67 (m, 1H), 1.65-1.41 (m, 2H), 1.38-1.19 (m, 3H). MS (ESI, m/e) [M+H]⁺ 648.5.

### Example 224: 6-(2-((2-oxabicyclo[2.1.1]hexan-4-yl)methoxy)-4-(3,8-diaza bicyclo[3.2.1]octan-3-yl)-6-chloro-8-fluoroquinazolin-7-yl)-4-methyl-5-(trifluoromethyl)pyridin-2-amine

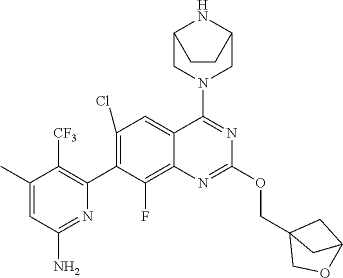 [SMILES: Cc1cc(N)nc(-c2c(Cl)cc3c(N4CC5CCC(C4)N5)nc(OCC45COC(C4)C5)nc3c2F)c1C(F)(F)F]

Example 224 was prepared by similar procedure as described in Example 206 by replacing 3-(hydroxymethyl)tetrahydrofuran-3-carbonitrile with (2-oxabicyclo [2.1.1]hexan-4-yl)methanol to give the title product (7.9 mg). ¹H NMR (400 MHz, DMSO-d₆) δ 7.82 (s, 1H), 6.85 (s, 2H), 6.49 (s, 1H), 4.68-4.57 (m, 2H), 4.49 (s, 1H), 4.31 (d, J=12.1 Hz, 1H), 4.18 (d, J=12.1 Hz, 1H), 3.72-3.38 (m, 6H), 2.67 (s, 1H), 2.37 (s, 3H), 1.82 (d, J=4.7 Hz, 2H), 1.61 (m, 4H), 1.49 (m, 2H). MS (ESI, m/e) [M+H]⁺ 579.10.

### Example 225: 5-(((7-(6-amino-4-methyl-3-(trifluoromethyl)pyridin-2-yl)-4-(3,8-diazabicyclo[3.2.1]octan-3-yl)-6-chloro-8-fluoroquinazolin-2-yl)oxy)methyl)-5-methylpiperidin-2-one

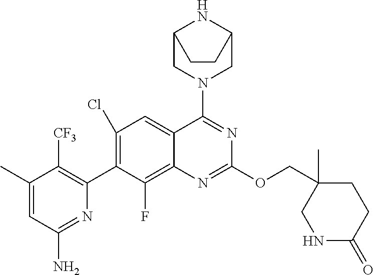 [SMILES: Cc1cc(N)nc(-c2c(Cl)cc3c(N4CC5CCC(C4)N5)nc(OCC4(C)CCC(=O)NC4)nc3c2F)c1C(F)(F)F]

Example 225 was prepared by similar procedure as described in Example 206 by replacing 3-(hydroxymethyl)tetrahydrofuran-3-carbonitrile with 5-(hydroxymethyl)-5-methyl piperidin-2-one to give the title product (3.0 mg). ¹H NMR (300 MHz, DMSO-d₆) δ 7.81 (s, 1H), 7.40 (s, 1H), 6.83 (s, 2H), 6.49 (s, 1H), 4.40-4.06 (m, 4H), 3.62-3.47 (m, 4H), 3.15 (d, J=13.0 Hz, 2H), 2.96 (d, J=12.2 Hz, 1H), 2.37 (s, 3H), 2.18 (t, J=6.7 Hz, 2H), 1.76 (dd, J=14.0, 6.9 Hz, 1H), 1.71-1.50 (m, 5H), 1.07 (s, 3H). (ESI, m/e) [M+H]⁺ 608.10.

### Example 226: 1-(3-(((7-(6-amino-4-methyl-3-(trifluoromethyl)pyridin-2-yl)-4-(3,8-diazabicyclo[3.2.1]octan-3-yl)-6-chloro-8-fluoroquinazolin-2-yl)oxy)methyl) azetidin-1-yl)ethan-1-one

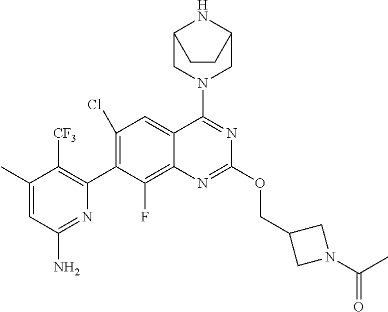 [SMILES: CC(=O)N1CC(COc2nc(N3CC4CCC(C3)N4)c3cc(Cl)c(-c4nc(N)cc(C)c4C(F)(F)F)c(F)c3n2)C1]

Example 226 was prepared by similar procedure as described in Example 206 by replacing 3-(hydroxymethyl)tetrahydrofuran-3-carbonitrile with 1-(3-(hydroxymethyl)azetidin-1-yl)ethan-1-one to give the title product (11.7 mg). ¹H NMR (300 MHz, DMSO-d₆) δ 7.82 (s, 1H), 6.85 (s, 2H), 6.49 (s, 1H), 4.46 (d, J=6.7 Hz, 2H), 4.35-4.12 (m, 3H), 4.00-3.90 (m, 2H), 3.72-3.59 (m, 1H), 3.58-3.41 (m, 3H), 3.07-2.92 (m, 2H), 2.39-2.35 (m, 3H), 2.31-2.24 (m, 1H), 1.74 (s, 3H), 1.66-1.56 (m, 4H). MS(ESI, m/e) [M+H]⁺ 616.10.

### Example 227: (1-(((7-(6-amino-4-methyl-3-(trifluoromethyl)pyridin-2-yl)-4-(3,8-diazabicyclo[3.2.1]octan-3-yl)-6-chloro-8-fluoroquinazolin-2-yl)oxy)methyl)-3-fluorocyclo butyl)methanol

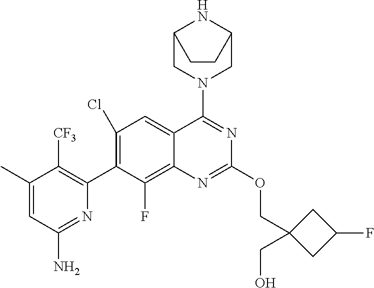 [SMILES: Cc1cc(N)nc(-c2c(Cl)cc3c(N4CC5CCC(C4)N5)nc(OCC4(CO)CC(F)C4)nc3c2F)c1C(F)(F)F]

Example 227 was prepared by similar procedure as described in Example 206 by replacing 3-(hydroxymethyl)tetrahydrofuran-3-carbonitrile with (3-fluorocyclobutane-1,1-diyl) dimethanol to give the title product (12.4 mg). ¹H NMR (400 MHz, DMSO-d₆) δ 7.81 (s, 1H), 6.84 (s, 2H), 6.49 (s, 1H), 5.30-4.96 (m, 1H), 4.88 (q, J=5.2 Hz, 1H), 4.41-4.22 (m, 3H), 4.17 (d, J=12.1 Hz, 1H), 3.57-3.39 (m, 6H), 2.76-2.58 (m, 1H), 2.37 (d, J=1.2 Hz, 3H), 2.34-2.21 (m, 2H), 2.21-2.02 (m, 2H), 1.79-1.49 (m, 4H). (ESI, m/e) [M+H]⁺ 599.10.

### Example 228: 3-(((7-(6-amino-4-methyl-3-(trifluoromethyl)pyridin-2-yl)-4-(3,8-diazabicyclo[3.2.1]octan-3-yl)-6-chloro-8-fluoroquinazolin-2-yl)oxy)methyl)thietane 1,1-dioxide

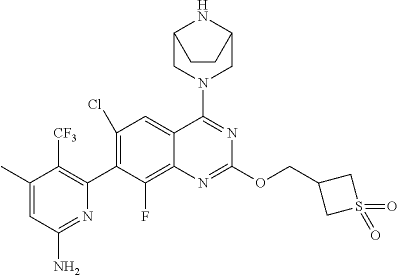 [SMILES: Cc1cc(N)nc(-c2c(Cl)cc3c(N4CC5CCC(C4)N5)nc(OCC4CS(=O)(=O)C4)nc3c2F)c1C(F)(F)F]

Example 228 was prepared by similar procedure as described in Example 206 by replacing 3-(hydroxymethyl)tetrahydrofuran-3-carbonitrile with 3-(hydroxymethyl)thietane 1,1-dioxide to give the title product (10.7 mg). ¹H NMR (400 MHz, DMSO-d₆) δ 7.83 (s, 1H), 6.84 (s, 2H), 6.49 (s, 1H), 4.49 (dd, J=7.4, 2.5 Hz, 2H), 4.40-4.26 (m, 3H), 4.19 (d, J=12.0 Hz, 1H), 4.08 (dd, J=13.5, 6.0 Hz, 2H), 3.58-3.42 (m, 4H), 2.99 (dq, J=12.3, 4.8, 3.0 Hz, 1H), 2.59-2.27 (m, 2H), 1.68-1.54 (m, 3H). MS(ESI, m/e) [M+H]⁺ 601.05.

### Example 229: 6-(2-((2-oxabicyclo[2.2.2]octan-4-yl)methoxy)-4-(3,8-diaza bicyclo[3.2.1]octan-3-yl)-6-chloro-8-fluoroquinazolin-7-yl)-4-methyl-5-(trifluoromethyl)pyridin-2-amine

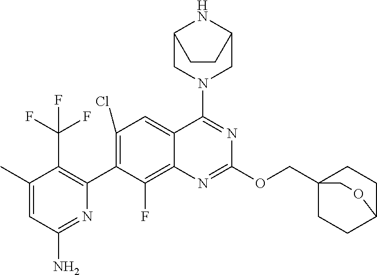 [SMILES: Cc1cc(N)nc(-c2c(Cl)cc3c(N4CC5CCC(C4)N5)nc(OCC45CCC(CC4)OC5)nc3c2F)c1C(F)(F)F]

Example 229 was prepared by similar procedure as described in Example 206 by replacing 3-(hydroxymethyl)tetrahydrofuran-3-carbonitrile with (2-oxabicyclo [2.2.2]octan-4-yl)methanol to give the title product (7.9 mg). 1H NMR (400 MHz, DMSO-d₆) δ 7.80 (s, 1H), 6.83 (s, 2H), 6.49 (s, 1H), 4.29 (d, J=12.0 Hz, 1H), 4.16 (d, J=11.7 Hz, 1H), 4.00 (q, J=10.9 Hz, 2H), 3.79-3.65 (m, 3H), 3.63-3.39 (m, 5H), 2.37 (s, 3H), 2.05-1.45 (m, 12H). MS(ESI, m/e) [M+H]⁺ 607.15.

### Example 230: (3-(((7-(6-amino-4-methyl-3-(trifluoromethyl)pyridin-2-yl)-4-(3,8-diazabicyclo[3.2.1]octan-3-yl)-6-chloro-8-fluoroquinazolin-2-yl)oxy)methyl)tetrahydrofuran-3-yl)methanol

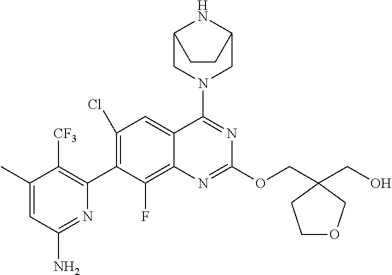 [SMILES: Cc1cc(N)nc(-c2c(Cl)cc3c(N4CC5CCC(C4)N5)nc(OCC4(CO)CCOC4)nc3c2F)c1C(F)(F)F]

Synthetic Route

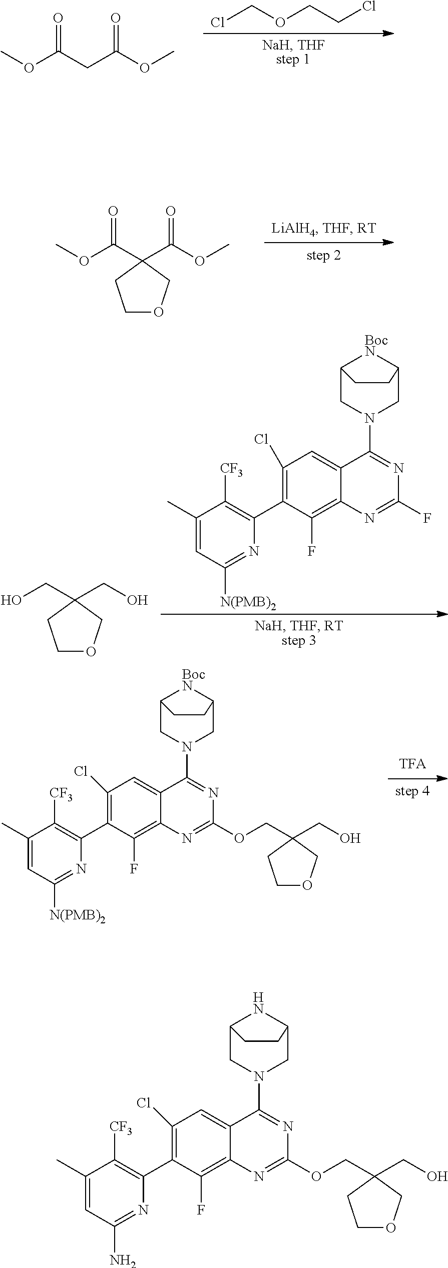

### Step 1: dimethyl dihydrofuran-3,3(2H)-dicarboxylate

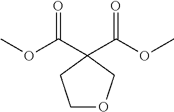

To a stirred solution of dimethyl malonate (2.0 g, 15.1 mmol) in THF (20 mL) was added NaH (1.21 g, 30.2 mmol, 60% in oil) at 0° C. The resulting mixture was stirred for 1 h at 0° C. Then 1-chloro-2-(chloromethoxy) ethane (2.34 g, 18.1 mmol) was added at 0° C. The resulting mixture was stirred for 5 h at rt. The mixture was quenched by H₂O and extracted with 2×30 mL of ethyl acetate. The organic phase was concentrated under reduced pressure. The crude product was purified by combi-Flash to give the title product (0.8 g).

### Step 2: (tetrahydrofuran-3,3-diyl)dimethanol

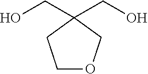

To a stirred solution of dimethyl dihydrofuran-3,3(2H)-dicarboxylate (100 mg, 0.53 mmol) in THF (2 mL) was added LiAlH₄ (40 mg, 1.05 mmol) at 0° C. The resulting mixture was stirred 4 h at rt. The mixture was quenched by Na₂SO₄-10H₂O at 0° C. The resulting mixture was concentrated under reduced pressure. The crude product was purified by combi-Flash to give the title product (30 mg).

Example 230 was prepared by similar procedure as described in Example 206 by replacing 3-(hydroxymethyl)tetrahydrofuran-3-carbonitrile with (tetrahydrofuran-3,3-diyl) dimethanol to give the title product (6.9 mg). ¹H NMR (400 MHz, CD₃OD) δ 7.83 (s, 1H), 6.60 (s, 1H), 4.51-4.38 (m, 4H), 3.96-3.82 (m, 2H), 3.77-3.56 (m, 8H), 2.44 (s, 3H), 1.97-1.85 (m, 2H), 1.85-1.75 (m, 4H). MS (ESI, m/e) [M+H]⁺ 597.10.

### Example 231: 6-(2-((3-oxabicyclo[3.1.0]hexan-6-yl)methoxy)-4-(3,8-diaza bicyclo[3.2.1]octan-3-yl)-6-chloro-8-fluoroquinazolin-7-yl)-4-methyl-5-(trifluoromethyl)pyridin-2-amine

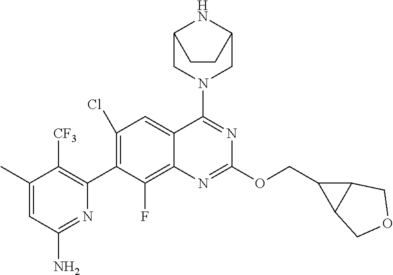 [SMILES: Cc1cc(N)nc(-c2c(Cl)cc3c(N4CC5CCC(C4)N5)nc(OCC4C5COCC54)nc3c2F)c1C(F)(F)F]

Example 231 was prepared by similar procedure as described in Example 206 by replacing 3-(hydroxymethyl)tetrahydrofuran-3-carbonitrile with (3-oxabicyclo[3.1.0]hexan-6-yl)methanol to give the title product (3.7 mg). ¹H NMR (400 MHz, DMSO-d₆) δ 8.23 (s, 1H), 7.81 (s, 1H), 6.85 (s, 2H), 6.49 (s, 1H), 4.49-4.34 (m, 1H), 4.34-4.25 (m, 1H), 4.25-4.12 (m, 2H), 3.94-3.70 (m, 3H), 3.62-3.55 (m, 3H), 3.54-3.41 (m, 2H), 2.37 (t, J=2.2 Hz, 3H), 1.90-1.84 (m, 1H), 1.75 (s, 1H), 1.65 (s, 4H), 1.46-1.32 (m, 1H), 1.16-1.05 (m, 1H). MS (ESI, m/e) [M+H]⁺ 579.10.

### Example 232: 1-(3-(((7-(6-amino-4-methyl-3-(trifluoromethyl)pyridin-2-yl)-4-(3,8-diazabicyclo[3.2.1]octan-3-yl)-6-chloro-8-fluoroquinazolin-2-yl)oxy)methyl)pyrrolidin-1-yl)ethan-1-one

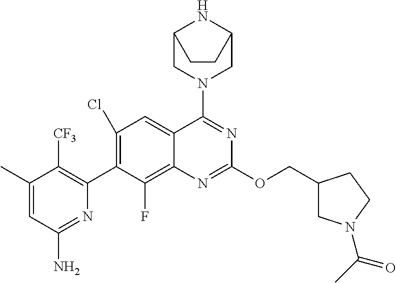 [SMILES: CC(=O)N1CCC(COc2nc(N3CC4CCC(C3)N4)c3cc(Cl)c(-c4nc(N)cc(C)c4C(F)(F)F)c(F)c3n2)C1]

Example 232 was prepared by similar procedure as described in Example 206 by replacing 3-(hydroxymethyl)tetrahydrofuran-3-carbonitrile with 1-(3-(hydroxymethyl)pyrrolidin-1-yl)ethan-1-one to give the title product (6.2 mg, FA salt). ¹H NMR (400 MHz, DMSO-d₆) δ 8.22 (s, 1H), 7.83 (s, 1H), 6.85 (s, 2H), 6.49 (s, 1H), 4.29 (m, 4H), 3.71-3.37 (m, 7H), 3.34-3.07 (m, 2H), 2.37 (s, 1H), 2.41-2.25 (m, 3H), 2.15-1.95 (m, 1H), 1.93 (s, 3H), 1.82 (m, 1H), 1.76-1.59 (m, 4H). MS (ESI, m/e) [M+H]⁺ 608.10.

### Example 233: 3-((7-(6-amino-4-methyl-3-(trifluoromethyl)pyridin-2-yl)-4-(3,8-diazabicyclo[3.2.1]octan-3-yl)-6-chloro-8-fluoroquinazolin-2-yl)oxy)-2-methoxy-2-methyl propan-1-ol

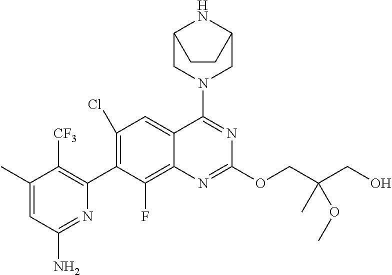 [SMILES: COC(C)(CO)COc1nc(N2CC3CCC(C2)N3)c2cc(Cl)c(-c3nc(N)cc(C)c3C(F)(F)F)c(F)c2n1]

Synthetic Route

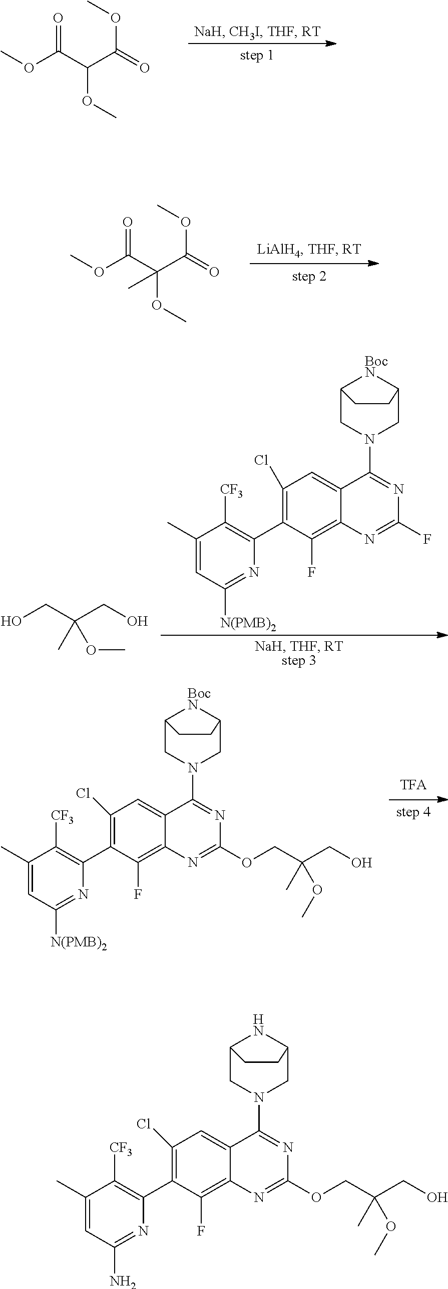

### Step 1: dimethyl 2-methoxy-2-methylmalonate

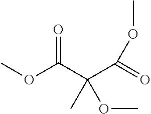

To a stirred solution of dimethyl 2-methoxymalonate (3.0 g, 18.5 mmol) in THF (30 mL) was added NaH (1.48 g, 37.0 mmol, 60% in oil) at 0° C. The resulting mixture was stirred for 1 h at 0° C. Then CH₃I (3.9 g, 27.5 mmol) was added at 0° C. The resulting mixture was stirred overnight at rt. The mixture was quenched by H₂O and extracted with 2×30 mL of ethyl acetate. The organic phase was concentrated under reduced pressure. The crude product was purified by combi-Flash with the following conditions: Column, silica gel; mobile phase, PE:EA=100:0 increasing to PE:EA=90:10 within 10 min; Detector, 254/280 nm to give the title product (1.2 g).

### Step 2: 2-methoxy-2-methylpropane-1,3-diol

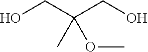

To a stirred solution of dimethyl 2-methoxy-2-methylmalonate (600 mg, 3.4 mmol) in THF (10 mL) was added LiAlH₄ (258 mg, 6.8 mmol) at 0° C. The resulting mixture was stirred 4 h at rt. The mixture was quenched by Na₂SO₄.10H₂O at 0° C. The resulting mixture was concentrated under reduced pressure. The crude product was purified by combi-Flash with the following conditions (Column, silica gel; mobile phase, DCM:MeOH=100:0 increasing to DCM:MeOH=90:10 within 15 min; Detector, 254/280 nm) to give the title product (70 mg).

Example 233 was prepared by similar procedure as described in Example 206 by replacing 3-(hydroxymethyl)tetrahydrofuran-3-carbonitrile with 2-methoxy-2-methylpropane-1,3-diol to give the title product (9.8 mg). ¹H NMR (400 MHz, CD₃OD) δ 7.83 (s, 1H), 6.59 (s, 1H), 4.55-4.40 (m, 3H), 4.35 (dd, J=11.2, 3.8 Hz, 1H), 3.69 (d, J=11.7 Hz, 1H), 3.64-3.52 (m, 5H), 3.34 (s, 3H), 2.44 (m, 3H), 1.86-1.77 (m, 4H), 1.27 (s, 3H). MS(ESI, m/e) [M+H]⁺ 585.15.

### Example 234: 1-(3-(((7-(6-amino-4-methyl-3-(trifluoromethyl) pyridine-2-yl)-4-(3,8-diazabicyclo[3.2.1]octan-3-yl)-6-chloro-8-fluoro quinazolin-2-yl)oxy)methyl)piperidin-1-yl)ethan-1-one

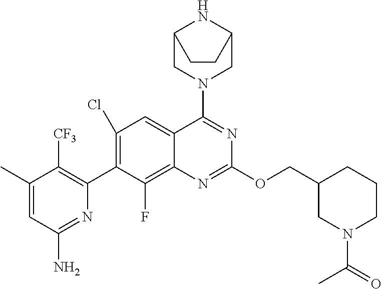 [SMILES: CC(=O)N1CCCC(COc2nc(N3CC4CCC(C3)N4)c3cc(Cl)c(-c4nc(N)cc(C)c4C(F)(F)F)c(F)c3n2)C1]

Synthetic Route

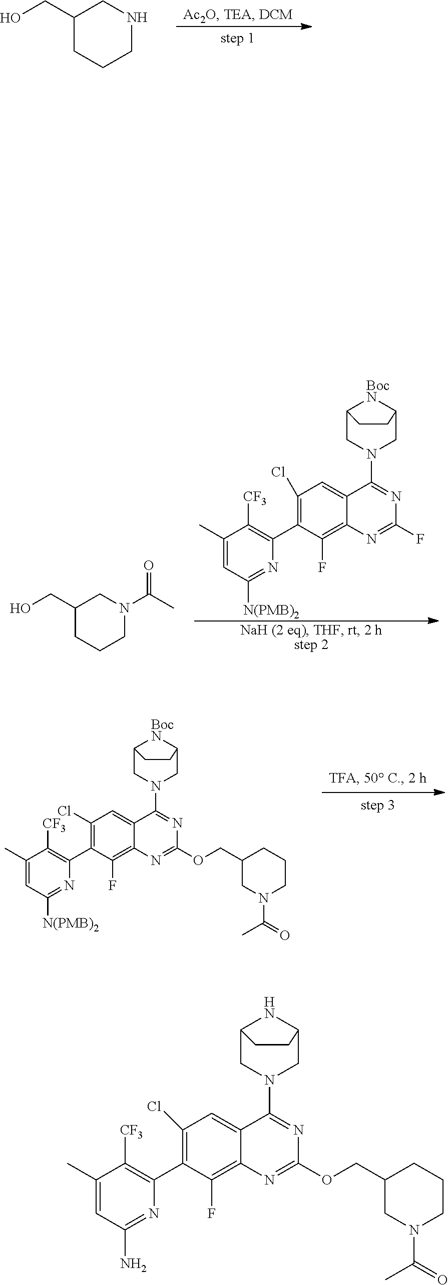

### Step 1: 1-(3-(hydroxymethyl)piperidin-1-yl)ethan-1-one

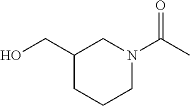

To a stirred solution of piperidin-3-ylmethanol (2 g, 17.39 mmol) in DCM (20 mL) was added Ac₂O (2.6 g, 25.49 mmol) and TEA (12.1 mL, 20.59 mmol) dropwise at 0° C. The resulting mixture was stirred for 2 h at rt. Then, the resulting mixture was concentrated under reduced pressure. The residue was purified by combi-flash (PE/EA=1:1) to give the title product (1.5 g, 56% yield). ¹H NMR (300 MHz, CDCl₃) δ 4.33-3.39 (m, 4H), 3.35-2.72 (m, 3H), 2.11 (s, 3H), 1.93-1.61 (m, 3H), 1.49 (s, 1H). MS(ESI, m/e) [M+H]⁺ 158.10.

Example 234 was prepared by similar procedure as described in Example 206 by replacing 3-(hydroxymethyl)tetrahydrofuran-3-carbonitrile with 1-(3-(hydroxymethyl)piperidin-1-yl)ethan-1-one to give the title product (11.2 mg, FA salt). ¹H NMR (300 MHz, DMSO-d₆) δ 8.22 (s, 1H), 7.82 (s, 1H), 6.85 (s, 2H), 6.49 (s, 1H), 4.49-4.01 (m, 6H), 3.95-3.59 (m, 5H), 3.08-2.95 (m, 1H), 2.87-2.65 (m, 1H), 2.36 (d, J=1.2 Hz, 3H), 1.98 (d, J=6.0 Hz, 3H), 1.92-1.77 (m, 2H), 1.76-1.53 (m, 5H), 1.51-1.24 (in, 2H). MS(ESI, m/e) [M+H]⁺ 622.10.

### Example 235: 4-(4-(3,8-diazabicyclo[3.2.1]octan-3-yl)-8-fluoro-2-((1-methyl-2-oxabicyclo[2.1.1]hexan-4-yl)methoxy)-6-(trifluoromethyl)quinazolin-7-yl)-2-amino-7-fluoro benzo[b]thiophene-3-carbonitrile

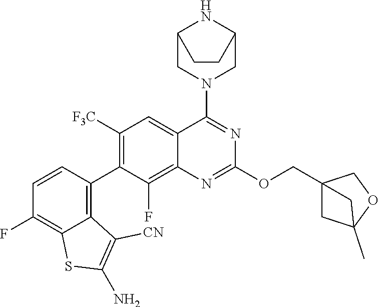 [SMILES: CC12CC(COc3nc(N4CC5CCC(C4)N5)c4cc(C(F)(F)F)c(-c5ccc(F)c6sc(N)c(C#N)c56)c(F)c4n3)(CO1)C2]

Example 235 was prepared by similar procedure as described in Example 236 by replacing (2-oxabicyclo[2.1.1]hexan-4-yl)methanol with (1-methyl-2-oxabicyclo[2.1.1]hexan-4-yl)methanol to give the title product (7.0 mg). ¹H NMR (500 MHz, CD₃OD) δ 8.11 (s, 1H), 7.22-7.20 (m, 1H), 7.03-7.00 (m, 1H), 4.77-4.71 (m, 2H), 4.68-4.53 (m, 2H), 3.94-3.73 (m, 6H), 1.98 (s, 4H), 1.81 (s, 2H), 1.64-1.62 (m, 2H), 1.42 (s, 3H). MS (ESI, m/e) [M+1]⁺643.4.

### Example 236: 4-(2-((2-oxabicyclo[2.1.1]hexan-4-yl)methoxy)-4-(3,8-diaza bicyclo[3.2.1]octan-3-yl)-8-fluoro-6-(trifluoromethyl)quinazolin-7-yl)-2-amino-7-fluorobenzo [b]thiophene-3-carbonitrile

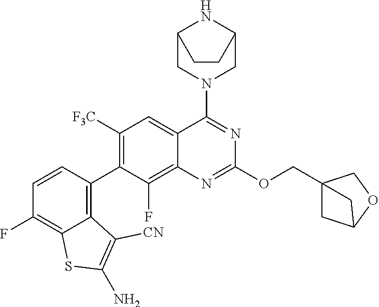 [SMILES: N#Cc1c(N)sc2c(F)ccc(-c3c(C(F)(F)F)cc4c(N5CC6CCC(C5)N6)nc(OCC56COC(C5)C6)nc4c3F)c12]

Synthetic Route

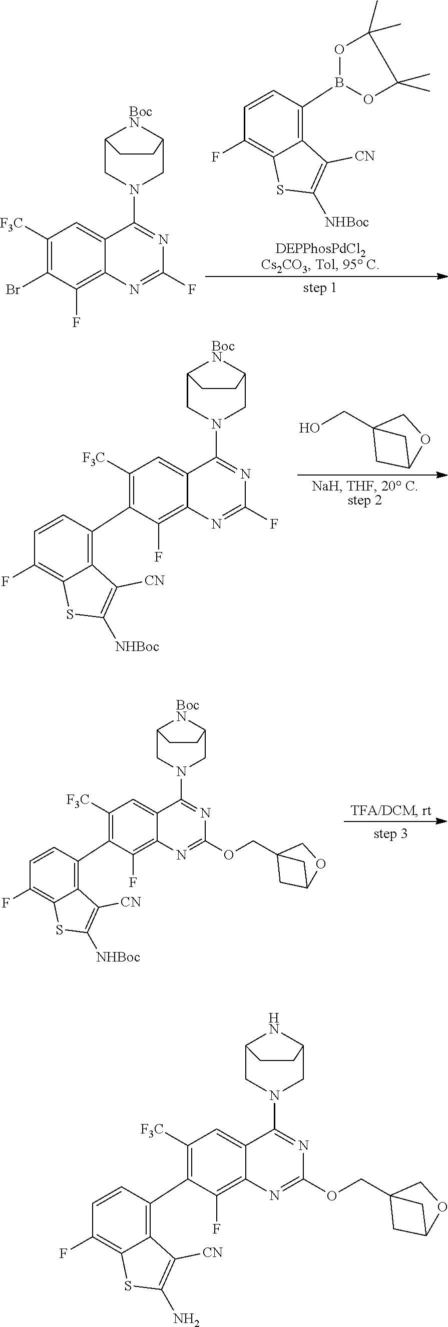

### Step 1: tert-butyl 3-(7-(2-((tert-butoxycarbonyl)amino)-3-cyano-7-fluorobenzo[b]thiophen-4-yl)-2,8-difluoro-6-(trifluoromethyl)quinazolin-4-yl)-3,8-diazabicyclo[3.2.1]octane-8-carboxylate

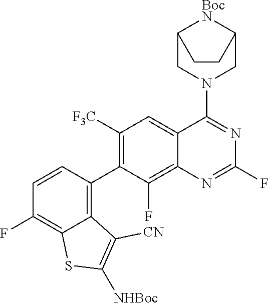 [SMILES: CC(C)(C)OC(=O)Nc1sc2c(F)ccc(-c3c(C(F)(F)F)cc4c(N5CC6CCC(C5)N6C(=O)OC(C)(C)C)nc(F)nc4c3F)c2c1C#N]

The mixture of tert-butyl 3-(7-bromo-2,8-difluoro-6-(trifluoromethyl)quinazolin-4-yl)-3,8-diazabicyclo[3.2.1]octane-8-carboxylate (2100 mg, 4.0 mmol), tert-butyl (3-cyano-7-fluoro-4-(4,4,5,5-tetramethyl-1,3,2-dioxaborolan-2-yl)benzo[b]thiophen-2-yl)carbamate (8.4 g, 20 mmol), PdCl₂DPEphos (2.9 g, 4.0 mmol) and Cs₂CO₃ (6.5 g, 20 mmol) in toluene/H₂O (50 mL/10 mL) was stirred at 95° C. for 2 hrs. After completion, the reaction mixture was purified by combi-flash (PE /EA=3:1) to give the title product (270 mg, 9.2% yield). MS (ESI, m/e) [M+1]⁺735.5.

### Step 2: tert-butyl 3-(2-((2-oxabicyclo[2.1.1]hexan-4-yl)methoxy)-7-(2-((tert-butoxycarbonyl)amino)-3-cyano-7-fluorobenzo[b]thiophen-4-yl)-8-fluoro-6-(trifluoromethyl) quinazolin-4-yl)-3,8-diazabicyclo[3.2.1]octane-8-carboxylate

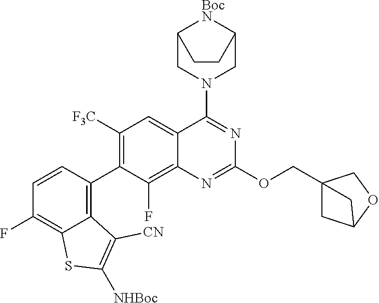 [SMILES: CC(C)(C)OC(=O)Nc1sc2c(F)ccc(-c3c(C(F)(F)F)cc4c(N5CC6CCC(C5)N6C(=O)OC(C)(C)C)nc(OCC56COC(C5)C6)nc4c3F)c2c1C#N]

To a solution of (2-oxabicyclo[2.1.1]hexan-4-yl)methanol (24 mg, 0.20 mmol) in THF (5 mL) was added sodium hydride (7.2 mg, 0.30 mmol) at room temperature. The resulting mixture was stirred at room temperature for 1 hrs. Then, tert-butyl 3-(7-(2-((tert-butoxy carbonyl)amino)-3-cyano-7-fluorobenzo[b]thiophen-4-yl)-2,8-difluoro-6-(trifluoromethyl) quinazolin-4-yl)-3,8-diazabicyclo[3.2.1]octane-8-carboxylate (73.5 mg, 0.10 mmol) was added to the mixture and the solution was stirred at room temperature for 1 hrs. After completion, the reaction mixture was diluted with EA (50 mL) and washed with brine (15 mL×3). The organic layer was dried over anhydrous Na₂SO₄, filtered and the filtrate was concentrated to give the residue. The residue was purified by Prep-TLC (PE:EA=1:1) to give the title product (60 mg, 72.3%). MS (ESI, m/e) [M+1]⁺829.5.

### Step 3: 4-(2-((2-oxabicyclo[2.1.1]hexan-4-yl)methoxy)-4-(3,8-diazabicyclo [3.2.1]octan-3-yl)-8-fluoro-6-(trifluoromethyl)quinazolin-7-yl)-2-amino-7-fluorobenzo[b] thiophene-3-carbonitrile

To a solution of tert-butyl 3-(2-((2-oxabicyclo[2.1.1]hexan-4-yl)methoxy)-7-(2-((tert-butoxycarbonyl)amino)-3-cyano-7-fluorobenzo[b]thiophen-4-yl)-8-fluoro-6-(trifluoro methyl)quinazolin-4-yl)-3,8-diazabicyclo[3.2.1]octane-8-carboxylate (60 mg, 0.072 mmol) in DCM (5 mL) was added TFA (5 mL) at room temperature. The resulting mixture was stirred at room temperature for 3 hrs. After completion, the reaction mixture was concentrated to give the residue. The residue was purified by prep-HPLC to give the title product (30 mg). ¹H NMR (500 MHz, CD₃OD) δ 8.11 (s, 1H), 7.23-7.19 (m, 1H), 7.02-6.99 (m, 1H), 4.79-4.73 (m, 2H), 4.57-4.44 (m, 3H), 3.74-3.63 (m, 6H), 1.96 (s, 2H), 1.87-1.76 (m, 4H), 1.64-1.61 (m, 2H). MS (ESI, m/e) [M+1]⁺629.5.

### Example 237: 3-(4-(3,8-diazabicyclo[3.2.1]octan-3-yl)-6-chloro-2-(((2R,7aS)-2-fluorotetrahydro-1H-pyrrolizin-7a(5H)-yl)methoxy) quinazolin-7-yl)-4,5-bis (trifluoromethyl) aniline

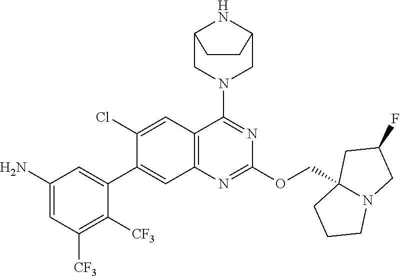 [SMILES: Nc1cc(-c2cc3nc(OC[C@@]45CCCN4C[C@H](F)C5)nc(N4CC5CCC(C4)N5)c3cc2Cl)c(C(F)(F)F)c(C(F)(F)F)c1]

Synthetic Route

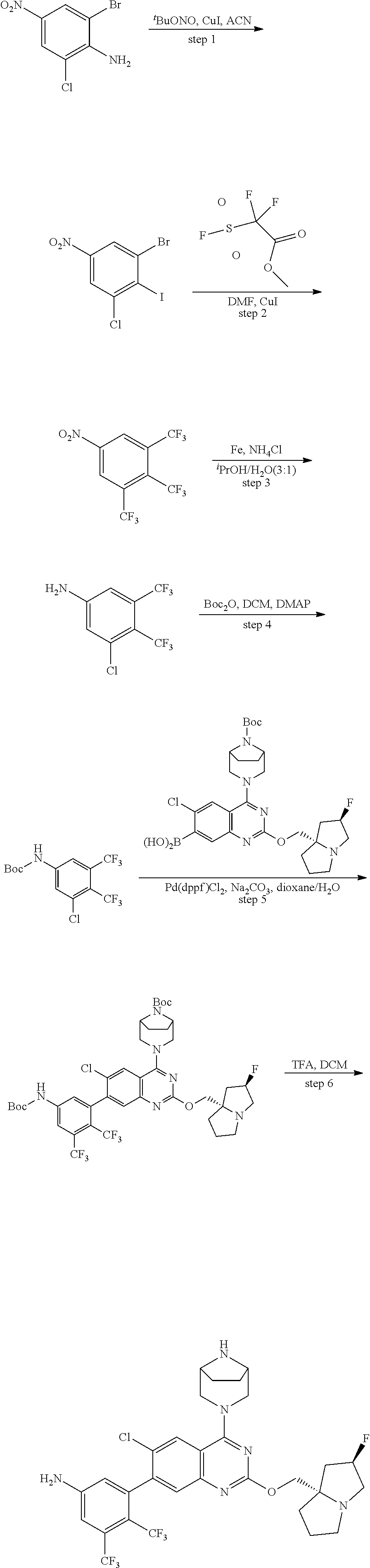

### Step 1:1-bromo-3-chloro-2-iodo-5-nitrobenzene

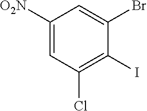 [SMILES: O=[N+]([O-])c1cc(Cl)c(I)c(Br)c1]

To a stirred solution of 2-bromo-6-chloro-4-nitroaniline (15 g, 59.65 mmol) and CuI (20.45 g, 107.37 mmol) in MeCN (300 ml) was added ^{t}BuONO (11.07 g, 107.37 mmol) dropwise at 0° C. The resulting mixture was stirred for 1 h at room temperature and 4 h at 60° C. Then, the resulting mixture was extracted with EtOAc (3×300 mL). The combined organic layers were washed with brine (2×500 mL) and dried over anhydrous Na₂SO₄. After filtration, the filtrate was concentrated under reduced pressure. The residue was purified by silica gel column chromatography, eluted with PE/EA (12:1) to give the title product (12 g, 56% yield).

### Step 2: 1-chloro-5-nitro-2,3-bis(trifluoromethyl)benzene

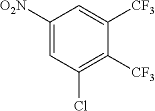 [SMILES: O=[N+]([O-])c1cc(Cl)c(C(F)(F)F)c(C(F)(F)F)c1]

To a mixture of 1-bromo-3-chloro-2-iodo-5-nitrobenzene (6 g, 16.55 mmol) and CuI (12.61 g, 66.23 mmol) in DMF (90 ml) was added methyl 2,2-difluoro-2-sulfoacetate (12.72 g, 66.23 mmol). The resulting mixture was stirred overnight at 60° C. Then, the resulting mixture was extracted with EtOAc (3×150 mL). The combined organic layers were washed with brine (3×300 mL), dried over anhydrous Na₂SO₄. After filtration, the filtrate was concentrated under reduced pressure. The residue was purified by silica gel column chromatography, eluted with PE/EA (12:1) to give the title product (2 g, 41%).

### Step 3: 3-chloro-4,5-bis(trifluoromethyl)aniline

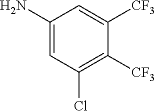 [SMILES: Nc1cc(Cl)c(C(F)(F)F)c(C(F)(F)F)c1]

To a solution of 1-chloro-5-nitro-2,3-bis(trifluoromethyl)benzene (4.27 g, 14.54 mmol) and NH₄Cl (1.95 g, 36.36 mmol) in i-PrOH/H₂O (55 ml) was added Fe (2.03 g, 36.36 mmol ). The resulting mixture was stirred overnight at 80° C. Then, the resulting mixture was filtered and the filtered cake was washed with MeOH (3×30 mL). The filtrate was concentrated under reduced pressure to remove the organic solvent. The residue was extracted with EtOAc (3×50 mL). The combined organic layers were washed with brine (100 ml), dried over anhydrous Na₂SO₄. After filtration, the filtrate was concentrated under reduced pressure. The residue was purified by silica gel column chromatography, eluted with PE/EA (5:1) to give the title product (3 g, 78%). MS (ESI, m/e) [M−H]-262.1.

### Step 4: tert-butyl (3-chloro-4,5-bis(trifluoromethyl)phenyl) carbamate

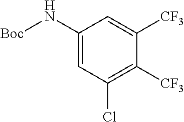 [SMILES: CC(C)(C)OC(=O)Nc1cc(Cl)c(C(F)(F)F)c(C(F)(F)F)c1]

To a mixture of 3-chloro-4,5-bis(trifluoromethyl)aniline (1.3 g, 4.93 mmol) and TEA (1.5 mL, 10.71 mmol) in DCM (20 ml) were added Boc₂O (1.61 g, 7.39 mmol) and DMAP (0.06 g, 0.49 mmol). The resulting mixture was stirred for 2 h at rt. The resulting mixture was concentrated under reduced pressure. The residue was purified by silica gel column chromatography, eluted with PE/EA (10:1) to give the title product (1 g, 56%). MS (ESI, m/e) [M−H]⁻ 362.1.

### Step 5: tert-butyl 3-(7-(5-((tert-butoxycarbonyl)amino)-2,3-bis(trifluoro methyl)phenyl)-6-chloro-2-(((2R,7aS)-2-fluorotetrahydro-1H-pyrrolizin-7a(5H)-yl) methoxy)quinazolin-4-yl)-3,8-diazabicyclo[3.2.1]octane-8-carboxylate

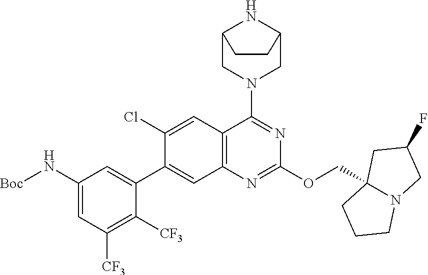 [SMILES: CC(C)(C)OC(=O)Nc1cc(-c2cc3nc(OC[C@@]45CCCN4C[C@H](F)C5)nc(N4CC5CCC(C4)N5)c3cc2Cl)c(C(F)(F)F)c(C(F)(F)F)c1]

To a solution of tert-butyl N-(3-chloro-4,5-bis(trifluoromethyl)phenyl)carbamate (130 mg, 0.35 mmol) and 2-(((2R,7aS)-2-fluoro-hexahydropyrrolizin-7a-yl)methoxy)-4-(8-(tert-butoxycarbonyl)-3,8-diazabicyclo [3.2.1]octan-3-yl)-6-chloro quinazolin-7-ylboronic acid (205.8 mg, 0.35 mmol) in 1,4-dioxane/H₂O (5 ml/1 ml) were added Na₂CO₃ (113.6 mg, 1.07 mmol) and Pd(dtbpf)Cl₂ (26.1 mg, 0.03 mmol). The resulting mixture was stirred for 2 h at 90° C. The resulting mixture was concentrated under reduced pressure. The residue was purified by Prep-TLC (CH₂Cl₂/MeOH 12:1) to give the title product (120 mg, 38% yield). MS (ESI, m/e) [M+H]⁺ 859.4.

### Step 6: 3-(4-(3,8-diazabicyclo[3.2.1]octan-3-yl)-6-chloro-2-(((2R,7aS)-2-fluoro tetrahydro-1H-pyrrolizin-7a(5H)-yl)methoxy) quinazolin-7-yl)-4,5-bis(trifluoromethyl) aniline

To a stirred solution of tert-butyl 3-(7-(5-((tert-butoxycarbonyl)amino)-2,3-bis(trifluoro methyl)phenyl)-6-chloro-2-(((2R,7aS)-2-fluorotetrahydro-1H-pyrrolizin-7a(5H)-yl) methoxy)quinazolin-4-yl)-3,8-diazabicyclo[3.2.1]octane-8-carboxylate (120 mg, 0.14 mmol) in DCM (2.5 ml) was added TFA (0.5 mL). The resulting mixture was stirred for 1 h at rt. The resulting mixture was concentrated under reduced pressure. The crude product was purified by Prep-HPLC to give the title product (3.4 mg, 3.7% yield). 1H NMR (400 MHz, CD₃OD) δ 8.01 (s, 1H), 7.40 (s, 1H), 7.19 (d, J=2.1 Hz, 1H), 6.61 (d, J=1.9 Hz, 1H), 5.30 (d, J=54.9 Hz, 1H), 4.47 (dd, J=20.8, 9.1 Hz, 2H), 4.22 (dt, J=31.3, 6.2 Hz, 2H), 3.69-3.55 (m, 4H), 3.27-3.14 (m, 3H), 3.06-2.93 (m, 1H), 2.40-2.07 (m, 3H), 2.06-1.93 (m, 2H), 1.91-1.76 (m, 5H). MS (ESI, m/e) [M+H]⁺ 659.1.

Methods

KRAS:SOS1 GDP TR-FRET Assay

This assay was used to identify compounds which competitively interact with the binding of KRAS protein to SOS1 in the presence of GDP. GST-tagged WT KRAS (amino acids 1-188), GST-tagged KRAS (amino acids 1-188) G12D and His-tagged SOS1 protein (amino acids 564-1049) was expressed in E. coli and purified in house. All protein and reaction solutions were prepared in assay buffer containing DPBS pH7.5, 0.1% BSA, and 0.05% Tween 20. Purified GST-tagged WT KRAS or KRAS G12D protein (37.5 nM final assay concentration) and GDP (Sigma, 10 μM final assay concentration) mixture, a serially diluted compound (top final concentration is 50 μM or 10 uM, 3-fold serially diluted, 10 points) and His-tagged SOS1 protein (18 nM final assay concentration) were added into the assay plates (384 well microplate, black, Corning) sequentially. Plates are incubated at 24° C. for 1 hr. Following the incubation, Mab Anti-GST-Tb cryptate (Cisbio) and Mab Anti GST-D2 (Cisbio) were added and further incubated at 24° C. for another 1 hr. The TR-FRET signals (ex337 nm, em665 nm/620 nm) were read on BMG PHERAstar FSX instrument. The inhibition percentage of KRAS protein binding with SOS1 in presence of increasing concentrations of compounds was calculated based on the ratio of fluorescence at 665 nm to that at 620 nm. The IC₅₀ value of each compound was calculated from fitting the data to the four-parameter logistic model by Dotmatics.

pERK Assay

AsPC-1 cell line was used in this study. Cells were maintained in RPMI-1640 supplemented with 10% fetal bovine serum (Thermo Fisher), 50 units/mL penicillin and streptomycin (Thermo Fisher) and kept at 37° C. in a humidified atmosphere of 5% CO₂ in air. Cells were reinstated from frozen stocks that were laid down within 30 passages from the original cells purchased. 30000 cells per well were seeded into a 96-well plate and incubated overnight. Cells were treated with a 10-point dilution series. The final compound concentration is from 0 to 10 μM. After 2 h compound treatment, cells were lysed, and the pERK1/2 (THR202/TYR204) level in the cell lysates was detected by HTRF kit (Cisbio). In brief, a total of 16 μL of cell lysate from each well of a 96-well plate was transferred to a 384-well white assay plate. Lysate from each well was incubated with 2 μL of Eu3+-cryptate (donor) labeled anti-phospho-ERK1/2 and 2 μL of D2 (acceptor) labeled anti-phospho-ERK1/2 antibodies (Cisbio) overnight in dark at room temperature. When donor and acceptor are in close proximity, excitation of the donor with laser triggers a Fluorescence Resonance Energy Transfer (FRET) towards the acceptor, which in turn fluoresces at 655 nm wavelength. FRET signals were measured using a PHERAstar FSX reader (BMG Labtech). IC₅₀ determination was performed by fitting the curve of percent inhibition versus the log of the inhibitor concentration using Dotmatics.

KRAS Probe Displacement Assay

This assay was used to identify compounds which bind to GDP-loaded KRAS protein and are able to displace a biotinylated probe occupying the KRAS binding site. GST-tagged GDP-loaded WT KRAS (amino acids 1-188) and GST-tagged GDP-loaded KRAS G12D (amino acids 1-188) were expressed in E. coli and purified in house. All protein and reaction solutions were prepared in assay buffer containing 50 mM HEPES pH7.5, 50 mM NaCl, 1 mM MgCl₂, 1 mM TCEP, 0.01% BSA, and 0.008% Brij-35. Purified WT KRAS (3 nM final concentration) or KRAS G12D protein (0.5 nM final concentration) was incubated with a 3-fold serially diluted compound in the assay plate (384 well microplate, black, Corning). Plates are incubated at 24° C. for 1 hr. Following the incubation, biotinylated probe 1 (60 nM final assay concentration) for WT KRAS and biotinylated probe 2 (4 nM final assay concentration) for KRAS G12D was added to the assay plate, respectively. After 1 hr incubation at 24° C., Mab Anti-GST-Tb cryptate (Cisbio) and Streptavidin-XL665 (Cisbio) were added and further incubated at 24° C. for another 1 hr. The TR-FRET signals (ex337 nm, em665 nm/620 nm) were read on BMG PHERAstar FSX instrument. The inhibition percentage of KRAS protein binding with biotinylated probe in presence of increasing concentrations of compounds was calculated based on the ratio of fluorescence at 665 nm to that at 620 nm. The IC₅₀ value of each compound was calculated from fitting the data to the four-parameter logistic model by Dotmatics.

TABLE 3
Data Analysis

| | | | KRAS^{G12D} | KRAS^{WT} | |
|---|---|---|---|---|---|
| | KRAS^{G12D}:SOS1 | KRAS^{WT}:SOS1 | Probe | Probe | |
| | GDP TR-FRET | GDP TR-FRET | Displacement | Displacement | |
| Example | assay | assay | Assay IC50 | Assay IC50 | pERK IC₅₀ |
| No. | IC₅₀ (nM) | IC₅₀ (nM) | (nM) | (nM) | (nM) |
| E1 | 1871 | 26863 | | | >10000 |
| E2 | 4847 | >50000 | | | N.D. |
| E3 | 68 | 3288 | | | >10000 |
| E4 | 39 | 1098 | | | >10000 |
| E5 | 20 | 402 | | | 2870 |
| E6 | 18 | 1056 | | | 7078 |
| E7 | 4.7 | 11 | | | 2635 |
| E8 | 6.7 | 50 | | | 1153 |
| E9 | 26 | 381 | | | 7687 |
| E10 | 5.7 | 387 | | | >10000 |
| E11 | 1.9 | 30 | | | 859 |
| E12 | 3.5 | 124 | | | 2022 |
| E13 | 8.3 | 537 | | | >10000 |
| E14 | 5.3 | 68 | | | 1155 |
| E15 | 4.4 | 83 | | | 574 |
| E16 | 2.8 | 27 | | | 1516 |
| E17 | 4.2 | 785 | | | 2878 |
| E18 | 2.5 | 35 | | | 528 |
| E19 | 10 | 566 | | | 3408 |
| E20 | 25 | >10000 | | | 7265 |
| E21 | 44 | 338 | | | >10000 |
| E22 | 27 | >3333 | | | 7695 |
| E23 | 3.9 | 146 | | | 5220 |
| E24 | 2.8 | 210 | | | 1267 |
| E25 | 2.2 | 88 | | | 860 |
| E26 | 2.9 | 140 | | | 819 |
| E27 | 2.9 | 57 | | | 518 |
| E28 | 3.0 | 30 | | | 8860 |
| E29 | 5.9 | 453 | | | 903 |
| E30 | 12 | 173 | | | 1807 |
| E31 | 11 | 1083 | | | 1895 |
| E32 | 81 | >10000 | | | N.D. |
| E33 | 6.1 | 143 | | | 890 |
| E34 | 15 | 1347 | | | >10000 |
| E35 | 22 | 3304 | 130 | 7300 | 4350 |
| E36 | 10 | 242 | 3.7 | 120 | 715 |
| E37 | 13 | 692 | 16 | 700 | 807 |
| E38 | 12 | 804 | 32 | 1400 | 1306 |
| E39 | 54 | 8158 | | | >10000 |
| E40 | 24 | 2207 | 41 | 1400 | >10000 |
| E41 | 11 | 438 | 11 | 230 | 1475 |
| E43 | 17 | 488 | 8.4 | 320 | 2553 |
| E49 | 4.8 | 35 | 3.1 | 49 | 84 |
| E50 | 7.6 | 140 | 4.0 | 160 | 312 |
| E51 | 6.5 | 61 | 1.1 | 33 | 202 |
| E52 | | | 0.36 | 3.0 | 48 |
| E53 | 5.1 | 132 | 1.2 | 47 | 380 |
| E54 | | | 0.35 | 7.5 | 205 |
| E55 | | | 0.25 | 2.7 | 59 |
| E56 | 21 | 370 | | | |
| E57 | >10000 | 130 | | | |
| E58 | | | 0.50 | 7.6 | 190 |
| E59 | | | 14 | 390 | 1400 |
| E60 | | | 0.27 | 2.6 | 34 |
| E61 | | | 2.6 | 240 | 2000 |
| E62 | | | 9.6 | 660 | 740 |
| E63 | | | 0.22 | 8.5 | 93 |
| E64 | | | 0.33 | 5.1 | 38 |
| E65 | | | 0.44 | 20.5 | 65 |
| E66 | | | 2.2 | 72 | 480 |
| E67 | | | 0.45 | 9.9 | 95 |
| E68 | | | 0.58 | 45 | 590 |
| E69 | | | 0.72 | 72 | 216 |
| E70 | | | 0.35 | 32 | 927 |
| E71 | | | 0.59 | 37.8 | 137 |
| E72 | | | 3.9 | 120 | 1075 |
| E73 | | | 0.57 | 15.5 | 68 |
| E74 | | | 0.42 | 4.8 | 349 |
| E75 | | | 0.23 | 1.8 | 40 |
| E76 | | | 0.12 | 4.8 | 58 |
| E77 | | | 0.52 | 33 | 122 |
| E78 | | | 0.99 | 101 | 159 |
| E79 | | | 1.1 | 178 | 153 |
| E80 | | | 0.66 | 46 | 307 |
| E81 | | | 6.9 | 990 | 2379 |
| E82 | | | 0.3 | 5 | 64 |
| E83 | | | 0.13 | 3.8 | 61 |
| E84 | | | 0.36 | 2.4 | 28 |
| E85 | | | 0.21 | 5.6 | 124 |
| E86 | | | 1.4 | 16 | 320 |
| E87 | | | 0.24 | 25 | 112 |
| E88 | | | 1.7 | 49 | 172 |
| E89 | | | 2.4 | 121 | 556 |

KRAS G12D NanoLuc Assay

HEK293 KRAS-G12D) NanoLuc cell pool was used in this study. The cells were stable expressing KRAS G12D HiBiT and LgBiT. Cells were maintained in DMEM supplemented with 1000 fetal bovine serum (Thermo Fisher), 50 units/mL penicillin and streptomycin (Thermo Fisher) and kept at 37° C. in a humidified atmosphere of 5% CO₂ in air. Cells were reinstated from frozen stocks that were laid down within 30 passages from the cell pool was constructed. 20000 cells per well were seeded a 96-well White with Clear Flat bottom plate for 4 h. Cells were treated with a 10-point dilution series. The final compound concentration is from 0 to 10 μM. After 24 h compound treatment, added 20 μL of CellTiter-Fluor reagent (Promega) to plates, shook for 2 min, incubated at least 30 min in 37° C. Added 25 μL of NanoGlo Live cell reagent (Promega) to the same plate. Shook by mini-shaker for 5 min. Measure Luminescence and Fluorescence (380-400 nmEx/505 nmEm) signals immediately using a PHERAstar FSX reader (BMG Labtech). DC50 determination was performed by filling the curve of percent inhibition versus the log of the inhibitor concentration using Excel and GraphPad Prism 8.

TABLE 4
Data Analysis

| | KRAS^{G12D} | KRAS^{G12D} |
|---|---|---|
| Example No. | NanoLuc DC50 (nM) | NanoLuc Dmax |
| E44 | 975 | 65% |
| E45 | 824 | 73% |
| E46 | 432 | 79% |
| E47 | 622 | 74% |
| E48 | 642 | 77% |

Western Blotting Assay

AsPC-1 cell line was used in this study. Cells were maintained in RPMI-1640 supplemented 10% fetal bovine serum (Thermo Fisher), 50 units/mL penicillin and streptomycin (Thermo Fisher) and kept at 37° C. In a humidified atmosphere of 5% CO₂ in air. Cells were reinstated from frozen stocks that were laid down within 30 passages from the original cells purchased. 400000 AsPC-1 cells per well in 1 mL culture medium were seed in the 12-well plate for 4 hours. Cells were treated with an appropriate dilution series of compounds. After 48 h compound treatment, medium was aspirated, the cells were washed with PBS, and then 30 μL of 1× protein lysis buffer (Cell Signaling Technology) containing protease inhibitors (Merck) and phosphatase inhibitors (Sigma) was added. The cells were lysed, and after centrifugation, the supernatants were quantified by BCA Protein assay Kit (Thermo Fisher). 4× loading Buffer (Thermo Fisher) was added to equal amounts of total protein from each sample and heated at 95° C. for 5 minutes. 30-50 g of cell lysates were loaded onto a 12% NuPAGE Bis-Tris Gel (Thermo Fisher), electro-transferred to NC membranes (Thermo Fisher), The membranes were blocked at least 1 hour with Blocking reagent (LI-COR), and then incubated overnight with anti-KRAS (LSBio, LS-C175665) or pERK (Cell Signaling Technology, 4370L) antibodies and as loading control anti-β-actin (Cell Signaling Technology, 3700S) or anti-GAPDH (Cell Signaling Technology, 97166S) at 4° C. with gentle shaking. The membranes were washed three times with TBST, and incubated at least 1 hour at room temperature with anti-mouse or anti-Rabbit secondary fluorescent antibody (Thermo Fisher, A32729; LI-COR, 926-32213). The membranes were washed three times in TBST, and one time in water. Immunoreactive bands were visualized by Odyssey CLx.

TABLE 5
Data Analysis protein level decrease

| Example No. | KRAS^{G12D} protein level decrease | Concentration (uM) |
|---|---|---|
| E44 | 70% | 3 |

KRAS^{G12D} Probe Displacement Assay IC50 data was collected with the assay provided herein and provided in the Table 6. The letter “A” refers to the KRAS G12D IC₅₀ value less than 50 nM; the letter “B” refers to the KRAS G12D IC₅₀ value between 50 nM and 100 nM; the letter “C” refers to the KRAS G12D IC₅₀ value between 100 nM and 1000 nM; the letter “D” refers to the KRAS G12D IC₅₀ value is more than 1000 nM.

TABLE 6

| Com- | | KRAS^{G12D} Probe |
|---|---|---|
| pound | | Displacement Assay |
| No. | Structure | IC50 (nM) |
| E90 | 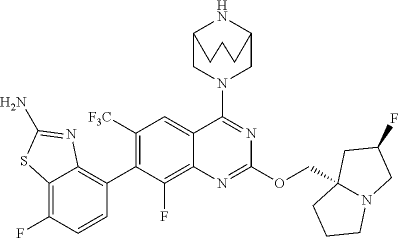 [SMILES: Nc1nc2c(-c3c(C(F)(F)F)cc4c(N5CC6CCCC(C5)N6)nc(OC[C@@]56CCCN5C[C@H](F)C6)nc4c3F)ccc(F)c2s1] | A |
| E91 | 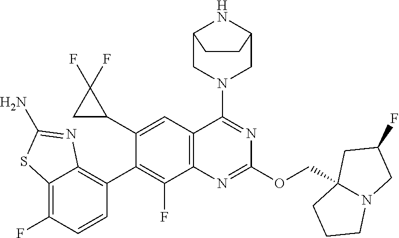 [SMILES: Nc1nc2c(-c3c(C4CC4(F)F)cc4c(N5CC6CCC(C5)N6)nc(OC[C@@]56CCCN5C[C@H](F)C6)nc4c3F)ccc(F)c2s1] | A |
| E92 | 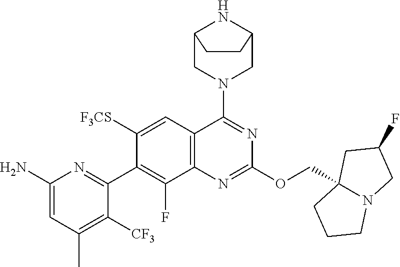 | A |
| E93 | 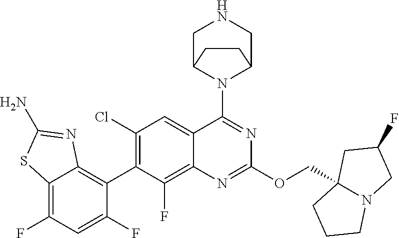 [SMILES: Nc1nc2c(-c3c(Cl)cc4c(N5C6CCC5CNC6)nc(OC[C@@]56CCCN5C[C@H](F)C6)nc4c3F)c(F)cc(F)c2s1] | A |
| E94 | 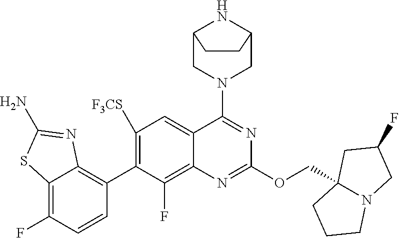 | A |
| E95 | 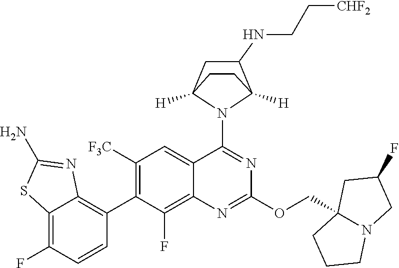 [SMILES: Nc1nc2c(-c3c(C(F)(F)F)cc4c(N5[C@@H]6CC[C@H]5C(NCCC(F)F)C6)nc(OC[C@@]56CCCN5C[C@H](F)C6)nc4c3F)ccc(F)c2s1] | A |
| E96 | 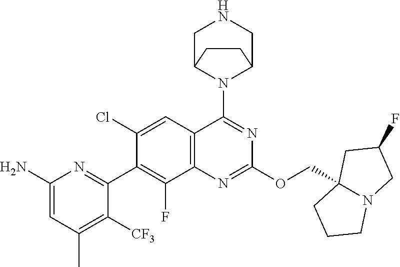 [SMILES: Cc1cc(N)nc(-c2c(Cl)cc3c(N4C5CCC4CNC5)nc(OC[C@@]45CCCN4C[C@H](F)C5)nc3c2F)c1C(F)(F)F] | A |
| E97 | 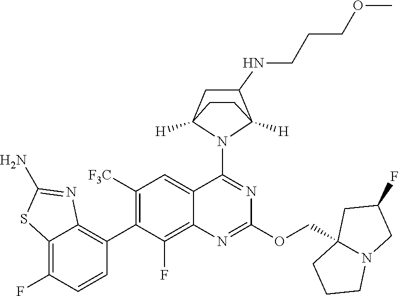 [SMILES: COCCCNC1C[C@H]2CC[C@@H]1N2c1nc(OC[C@@]23CCCN2C[C@H](F)C3)nc2c(F)c(-c3ccc(F)c4sc(N)nc34)c(C(F)(F)F)cc12] | A |
| E98 | 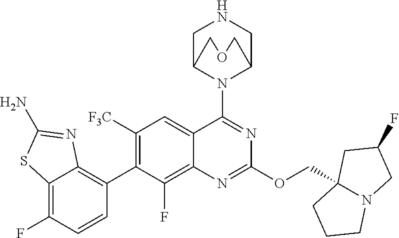 [SMILES: Nc1nc2c(-c3c(C(F)(F)F)cc4c(N5C6CNCC5COC6)nc(OC[C@@]56CCCN5C[C@H](F)C6)nc4c3F)ccc(F)c2s1] | A |
| E99 | 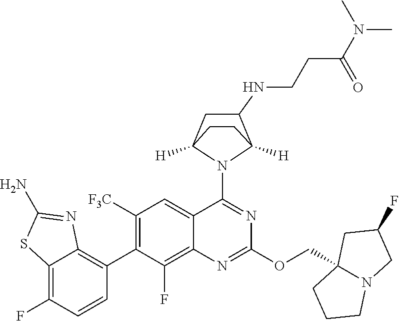 [SMILES: CN(C)C(=O)CCNC1C[C@H]2CC[C@@H]1N2c1nc(OC[C@@]23CCCN2C[C@H](F)C3)nc2c(F)c(-c3ccc(F)c4sc(N)nc34)c(C(F)(F)F)cc12] | A |
| E100 | 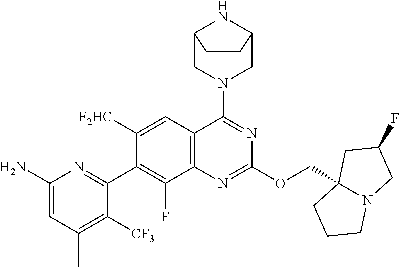 [SMILES: Cc1cc(N)nc(-c2c(C(F)F)cc3c(N4CC5CCC(C4)N5)nc(OC[C@@]45CCCN4C[C@H](F)C5)nc3c2F)c1C(F)(F)F] | A |
| E101 | 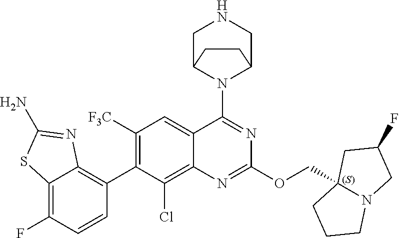 | A |
| E102 | 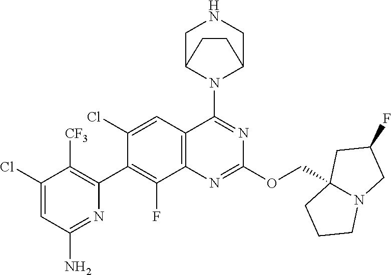 [SMILES: Nc1cc(Cl)c(C(F)(F)F)c(-c2c(Cl)cc3c(N4C5CCC4CNC5)nc(OC[C@@]45CCCN4C[C@H](F)C5)nc3c2F)n1] | A |
| E103 | 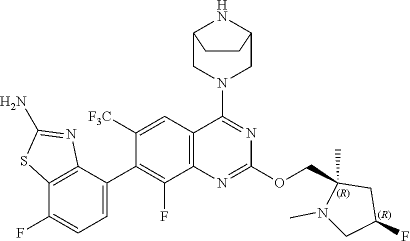 | A |
| E104 | 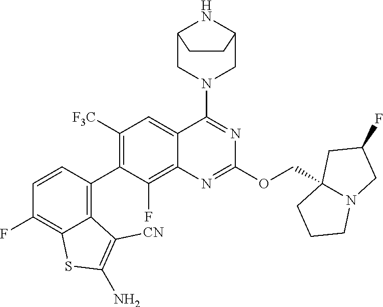 [SMILES: N#Cc1c(N)sc2c(F)ccc(-c3c(C(F)(F)F)cc4c(N5CC6CCC(C5)N6)nc(OC[C@@]56CCCN5C[C@H](F)C6)nc4c3F)c12] | A |
| E105 | 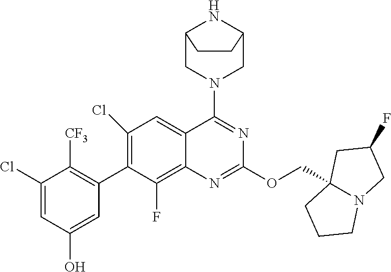 [SMILES: Oc1cc(Cl)c(C(F)(F)F)c(-c2c(Cl)cc3c(N4CC5CCC(C4)N5)nc(OC[C@@]45CCCN4C[C@H](F)C5)nc3c2F)c1] | A |
| E106 | 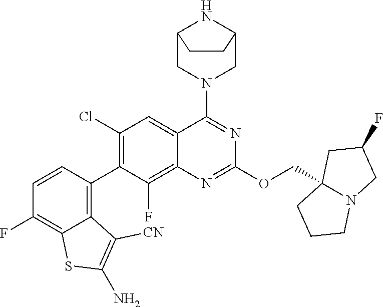 [SMILES: N#Cc1c(N)sc2c(F)ccc(-c3c(Cl)cc4c(N5CC6CCC(C5)N6)nc(OC[C@@]56CCCN5C[C@H](F)C6)nc4c3F)c12] | A |
| E107 | 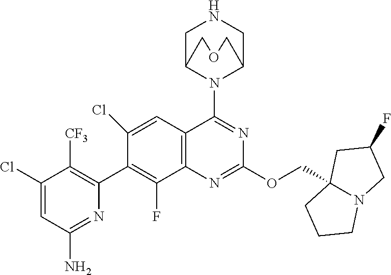 [SMILES: Nc1cc(Cl)c(C(F)(F)F)c(-c2c(Cl)cc3c(N4C5CNCC4COC5)nc(OC[C@@]45CCCN4C[C@H](F)C5)nc3c2F)n1] | A |
| E108 | 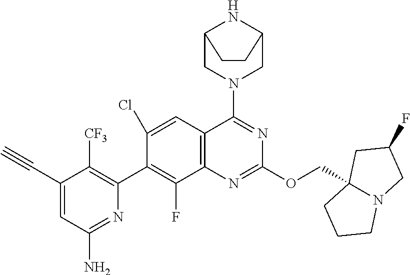 [SMILES: C#Cc1cc(N)nc(-c2c(Cl)cc3c(N4CC5CCC(C4)N5)nc(OC[C@@]45CCCN4C[C@H](F)C5)nc3c2F)c1C(F)(F)F] | A |
| E109 | 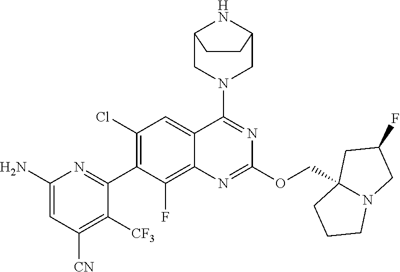 [SMILES: N#Cc1cc(N)nc(-c2c(Cl)cc3c(N4CC5CCC(C4)N5)nc(OC[C@@]45CCCN4C[C@H](F)C5)nc3c2F)c1C(F)(F)F] | A |
| E110 | 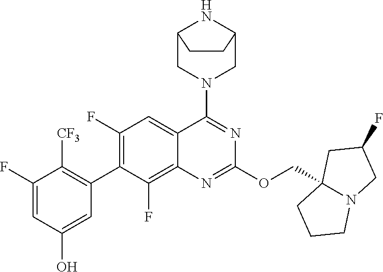 [SMILES: Oc1cc(F)c(C(F)(F)F)c(-c2c(F)cc3c(N4CC5CCC(C4)N5)nc(OC[C@@]45CCCN4C[C@H](F)C5)nc3c2F)c1] | A |
| E111 | 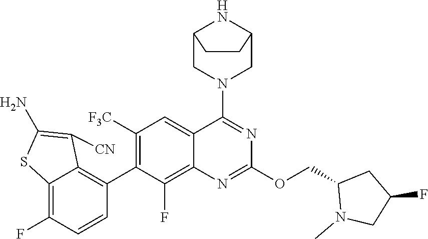 [SMILES: CN1C[C@H](F)C[C@H]1COc1nc(N2CC3CCC(C2)N3)c2cc(C(F)(F)F)c(-c3ccc(F)c4sc(N)c(C#N)c34)c(F)c2n1] | A |
| E112 | 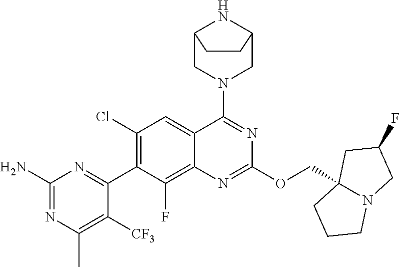 [SMILES: Cc1nc(N)nc(-c2c(Cl)cc3c(N4CC5CCC(C4)N5)nc(OC[C@@]45CCCN4C[C@H](F)C5)nc3c2F)c1C(F)(F)F] | A |
| E113 | 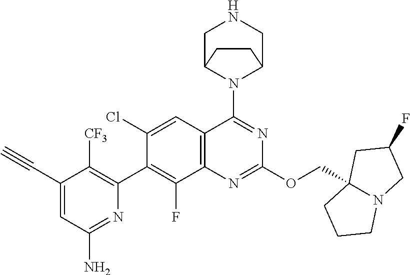 [SMILES: C#Cc1cc(N)nc(-c2c(Cl)cc3c(N4C5CCC4CNC5)nc(OC[C@@]45CCCN4C[C@H](F)C5)nc3c2F)c1C(F)(F)F] | A |
| E114 | 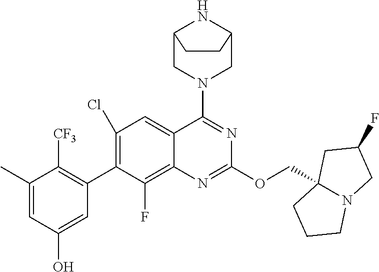 [SMILES: Cc1cc(O)cc(-c2c(Cl)cc3c(N4CC5CCC(C4)N5)nc(OC[C@@]45CCCN4C[C@H](F)C5)nc3c2F)c1C(F)(F)F] | A |
| E115 | 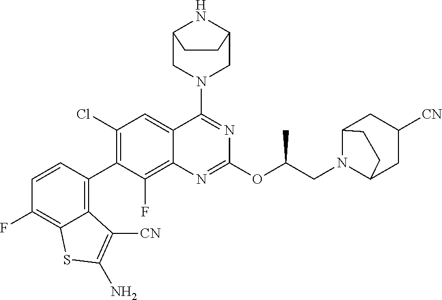 [SMILES: C[C@@H](CN1C2CCC1CC(C#N)C2)Oc1nc(N2CC3CCC(C2)N3)c2cc(Cl)c(-c3ccc(F)c4sc(N)c(C#N)c34)c(F)c2n1] | A |
| E116 | 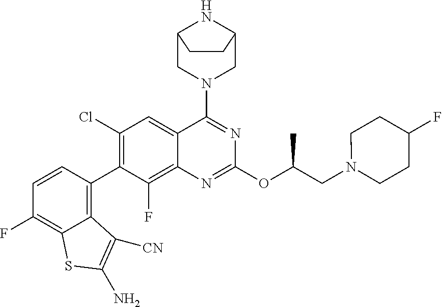 [SMILES: C[C@@H](CN1CCC(F)CC1)Oc1nc(N2CC3CCC(C2)N3)c2cc(Cl)c(-c3ccc(F)c4sc(N)c(C#N)c34)c(F)c2n1] | A |
| E117 | 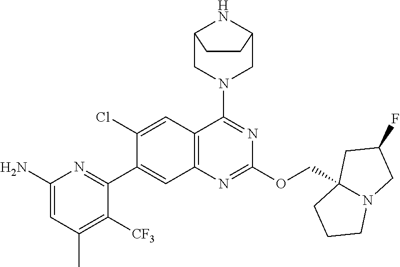 [SMILES: Cc1cc(N)nc(-c2cc3nc(OC[C@@]45CCCN4C[C@H](F)C5)nc(N4CC5CCC(C4)N5)c3cc2Cl)c1C(F)(F)F] | A |
| E118 | 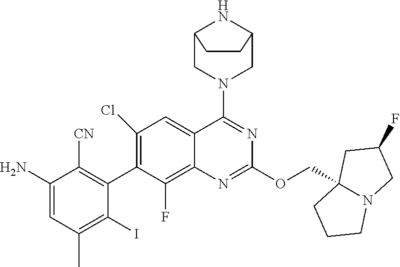 [SMILES: Cc1cc(N)c(C#N)c(-c2c(Cl)cc3c(N4CC5CCC(C4)N5)nc(OC[C@@]45CCCN4C[C@H](F)C5)nc3c2F)c1I] | A |
| E119 | 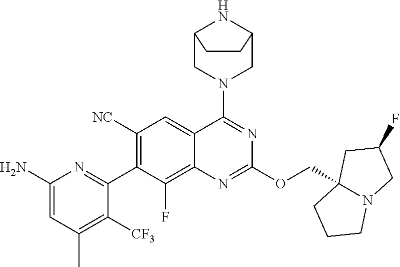 [SMILES: Cc1cc(N)nc(-c2c(C#N)cc3c(N4CC5CCC(C4)N5)nc(OC[C@@]45CCCN4C[C@H](F)C5)nc3c2F)c1C(F)(F)F] | A |
| E120 | 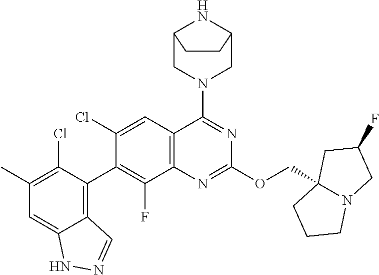 [SMILES: Cc1cc2[nH]ncc2c(-c2c(Cl)cc3c(N4CC5CCC(C4)N5)nc(OC[C@@]45CCCN4C[C@H](F)C5)nc3c2F)c1Cl] | A |
| E121 | 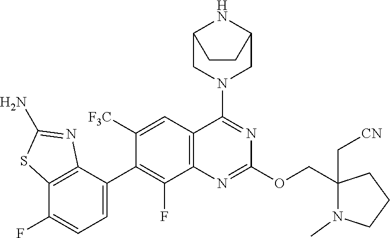 [SMILES: CN1CCCC1(CC#N)COc1nc(N2CC3CCC(C2)N3)c2cc(C(F)(F)F)c(-c3ccc(F)c4sc(N)nc34)c(F)c2n1] | A |
| E122 | 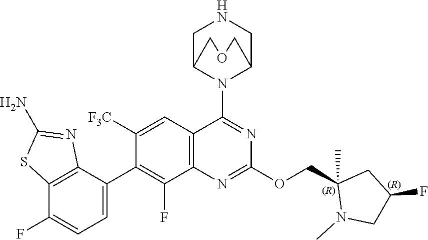 | A |
| E123 | 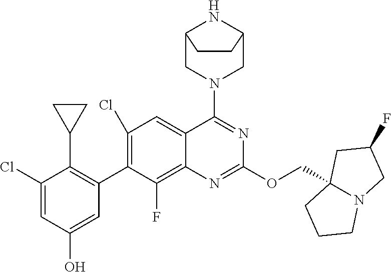 [SMILES: Oc1cc(Cl)c(C2CC2)c(-c2c(Cl)cc3c(N4CC5CCC(C4)N5)nc(OC[C@@]45CCCN4C[C@H](F)C5)nc3c2F)c1] | A |
| E124 | 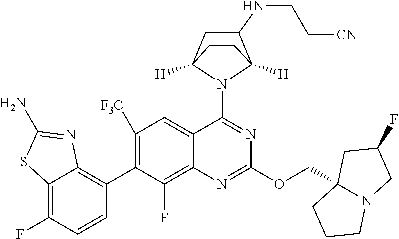 [SMILES: N#CCCNC1C[C@H]2CC[C@@H]1N2c1nc(OC[C@@]23CCCN2C[C@H](F)C3)nc2c(F)c(-c3ccc(F)c4sc(N)nc34)c(C(F)(F)F)cc12] | A |
| E125 | 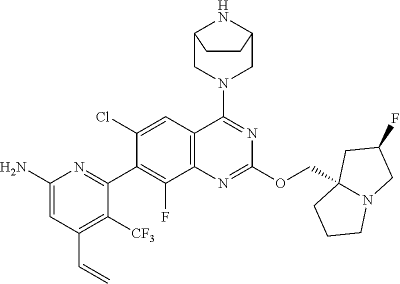 [SMILES: C=Cc1cc(N)nc(-c2c(Cl)cc3c(N4CC5CCC(C4)N5)nc(OC[C@@]45CCCN4C[C@H](F)C5)nc3c2F)c1C(F)(F)F] | A |
| E126 | 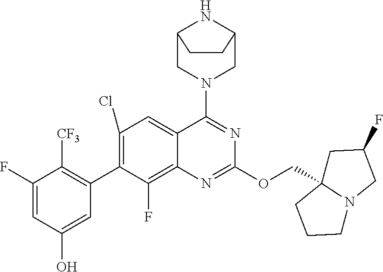 [SMILES: Oc1cc(F)c(C(F)(F)F)c(-c2c(Cl)cc3c(N4CC5CCC(C4)N5)nc(OC[C@@]45CCCN4C[C@H](F)C5)nc3c2F)c1] | A |
| E127 | 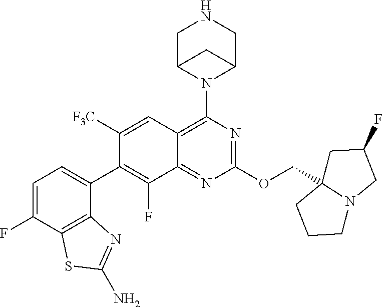 | A |
| E128 | 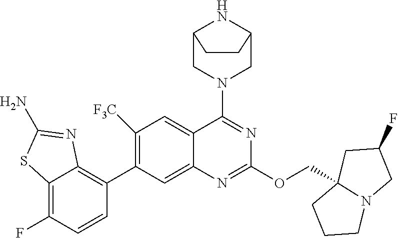 [SMILES: Nc1nc2c(-c3cc4nc(OC[C@@]56CCCN5C[C@H](F)C6)nc(N5CC6CCC(C5)N6)c4cc3C(F)(F)F)ccc(F)c2s1] | A |
| E129 | 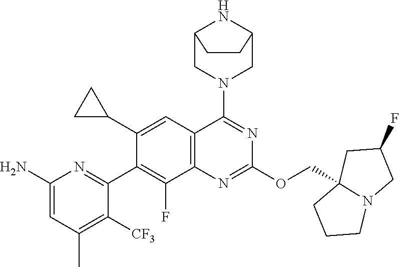 [SMILES: Cc1cc(N)nc(-c2c(C3CC3)cc3c(N4CC5CCC(C4)N5)nc(OC[C@@]45CCCN4C[C@H](F)C5)nc3c2F)c1C(F)(F)F] | A |
| E130 | 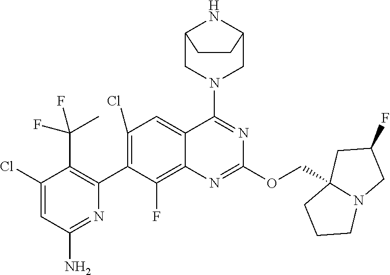 [SMILES: CC(F)(F)c1c(Cl)cc(N)nc1-c1c(Cl)cc2c(N3CC4CCC(C3)N4)nc(OC[C@@]34CCCN3C[C@H](F)C4)nc2c1F] | A |
| E131 | 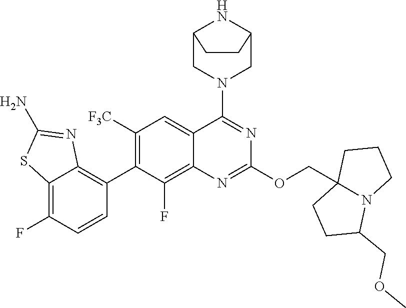 [SMILES: COCC1CCC2(COc3nc(N4CC5CCC(C4)N5)c4cc(C(F)(F)F)c(-c5ccc(F)c6sc(N)nc56)c(F)c4n3)CCCN12] | A |
| E132 | 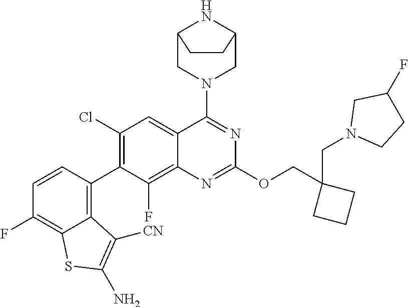 [SMILES: N#Cc1c(N)sc2c(F)ccc(-c3c(Cl)cc4c(N5CC6CCC(C5)N6)nc(OCC5(CN6CCC(F)C6)CCC5)nc4c3F)c12] | A |
| E133 | 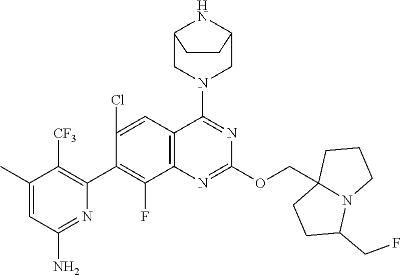 [SMILES: Cc1cc(N)nc(-c2c(Cl)cc3c(N4CC5CCC(C4)N5)nc(OCC45CCCN4C(CF)CC5)nc3c2F)c1C(F)(F)F] | A |
| E134 | 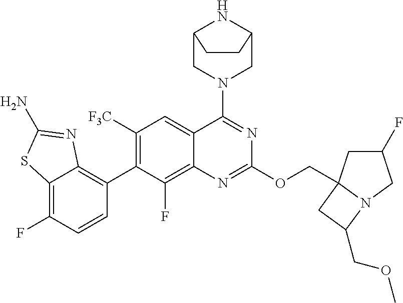 [SMILES: COCC1CC2(COc3nc(N4CC5CCC(C4)N5)c4cc(C(F)(F)F)c(-c5ccc(F)c6sc(N)nc56)c(F)c4n3)CC(F)CN12] | A |
| E135 | 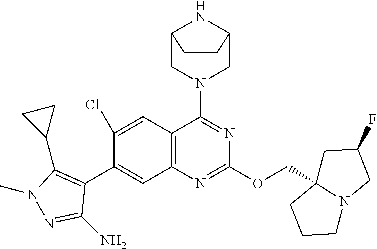 [SMILES: Cn1nc(N)c(-c2cc3nc(OC[C@@]45CCCN4C[C@H](F)C5)nc(N4CC5CCC(C4)N5)c3cc2Cl)c1C1CC1] | D |
| E136 | 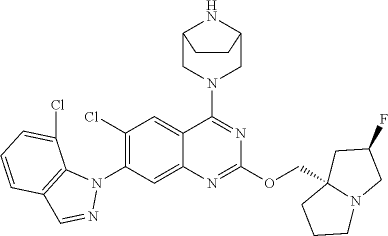 [SMILES: F[C@H]1CN2CCC[C@@]2(COc2nc(N3CC4CCC(C3)N4)c3cc(Cl)c(-n4ncc5cccc(Cl)c54)cc3n2)C1] | D |
| E137 | 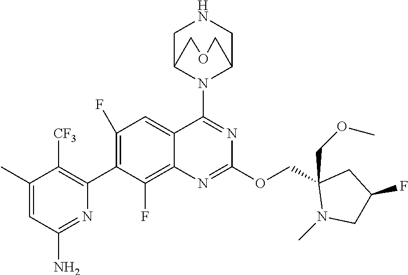 [SMILES: COC[C@@]1(COc2nc(N3C4CNCC3COC4)c3cc(F)c(-c4nc(N)cc(C)c4C(F)(F)F)c(F)c3n2)C[C@@H](F)CN1C] | C |
| E138 | 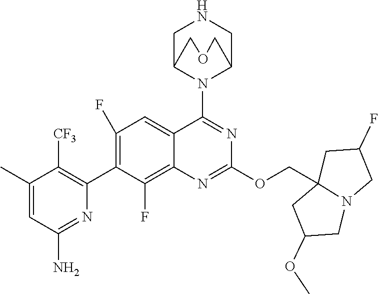 [SMILES: COC1CN2CC(F)CC2(COc2nc(N3C4CNCC3COC4)c3cc(F)c(-c4nc(N)cc(C)c4C(F)(F)F)c(F)c3n2)C1] | C |
| E139 | 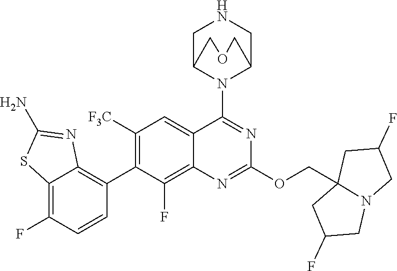 [SMILES: Nc1nc2c(-c3c(C(F)(F)F)cc4c(N5C6CNCC5COC6)nc(OCC56CC(F)CN5CC(F)C6)nc4c3F)ccc(F)c2s1] | A |
| E140 | 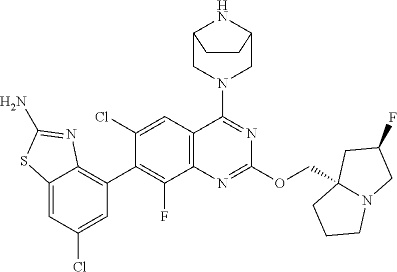 [SMILES: Nc1nc2c(-c3c(Cl)cc4c(N5CC6CCC(C5)N6)nc(OC[C@@]56CCCN5C[C@H](F)C6)nc4c3F)cc(Cl)cc2s1] | A |
| E141 | 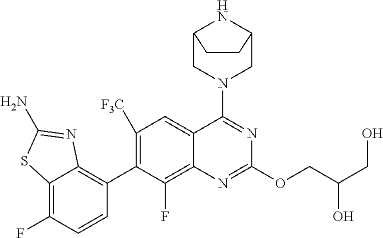 [SMILES: Nc1nc2c(-c3c(C(F)(F)F)cc4c(N5CC6CCC(C5)N6)nc(OCC(O)CO)nc4c3F)ccc(F)c2s1] | A |
| E142 | 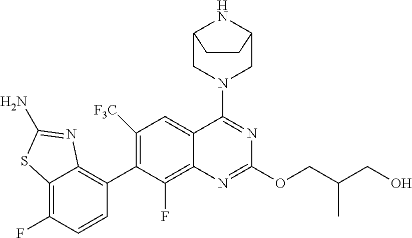 [SMILES: CC(CO)COc1nc(N2CC3CCC(C2)N3)c2cc(C(F)(F)F)c(-c3ccc(F)c4sc(N)nc34)c(F)c2n1] | A |
| E143 | 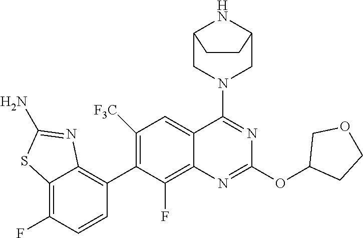 [SMILES: Nc1nc2c(-c3c(C(F)(F)F)cc4c(N5CC6CCC(C5)N6)nc(OC5CCOC5)nc4c3F)ccc(F)c2s1] | A |
| E144 | 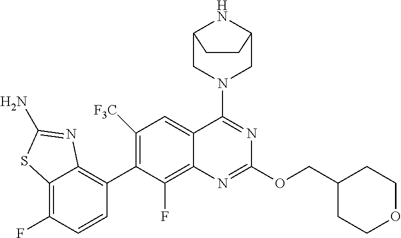 [SMILES: Nc1nc2c(-c3c(C(F)(F)F)cc4c(N5CC6CCC(C5)N6)nc(OCC5CCOCC5)nc4c3F)ccc(F)c2s1] | A |
| E145 | 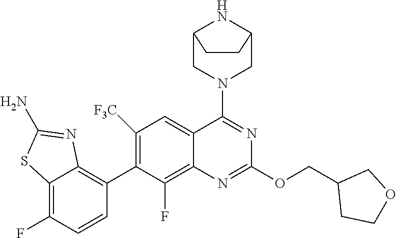 [SMILES: Nc1nc2c(-c3c(C(F)(F)F)cc4c(N5CC6CCC(C5)N6)nc(OCC5CCOC5)nc4c3F)ccc(F)c2s1] | A |
| E146 | 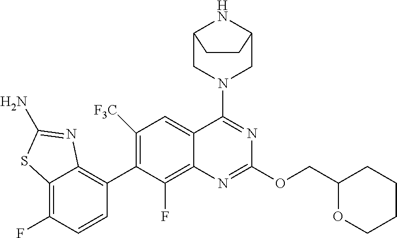 [SMILES: Nc1nc2c(-c3c(C(F)(F)F)cc4c(N5CC6CCC(C5)N6)nc(OCC5CCCCO5)nc4c3F)ccc(F)c2s1] | A |
| E147 | 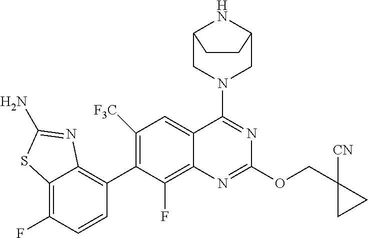 [SMILES: N#CC1(COc2nc(N3CC4CCC(C3)N4)c3cc(C(F)(F)F)c(-c4ccc(F)c5sc(N)nc45)c(F)c3n2)CC1] | A |
| E148 | 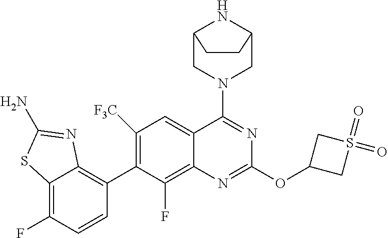 [SMILES: Nc1nc2c(-c3c(C(F)(F)F)cc4c(N5CC6CCC(C5)N6)nc(OC5CS(=O)(=O)C5)nc4c3F)ccc(F)c2s1] | A |
| E149 | 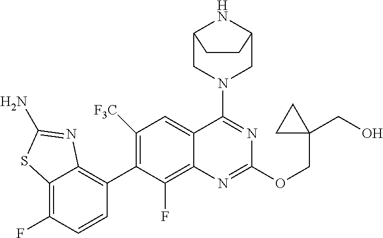 [SMILES: Nc1nc2c(-c3c(C(F)(F)F)cc4c(N5CC6CCC(C5)N6)nc(OCC5(CO)CC5)nc4c3F)ccc(F)c2s1] | A |
| E150 | 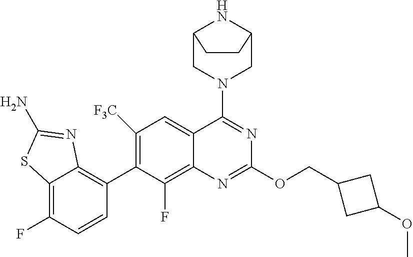 [SMILES: COC1CC(COc2nc(N3CC4CCC(C3)N4)c3cc(C(F)(F)F)c(-c4ccc(F)c5sc(N)nc45)c(F)c3n2)C1] | A |
| E151 | 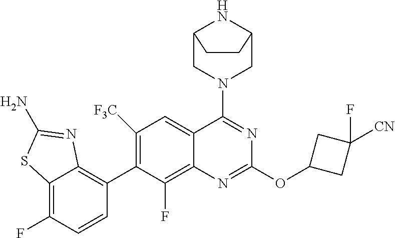 [SMILES: N#CC1(F)CC(Oc2nc(N3CC4CCC(C3)N4)c3cc(C(F)(F)F)c(-c4ccc(F)c5sc(N)nc45)c(F)c3n2)C1] | A |
| E152 | 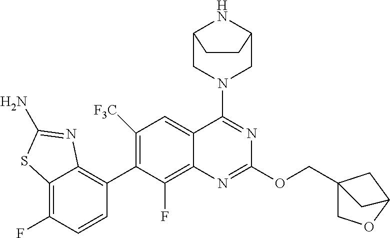 [SMILES: Nc1nc2c(-c3c(C(F)(F)F)cc4c(N5CC6CCC(C5)N6)nc(OCC56COC(C5)C6)nc4c3F)ccc(F)c2s1] | A |
| E153 | 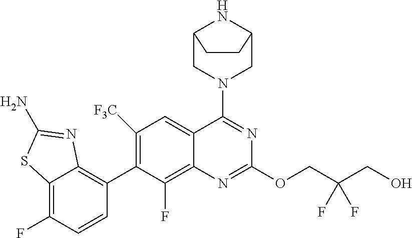 [SMILES: Nc1nc2c(-c3c(C(F)(F)F)cc4c(N5CC6CCC(C5)N6)nc(OCC(F)(F)CO)nc4c3F)ccc(F)c2s1] | A |
| E154 | 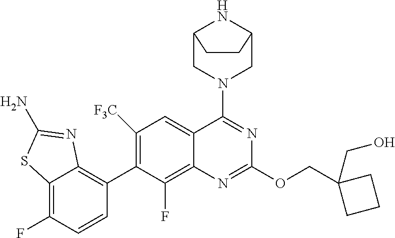 [SMILES: Nc1nc2c(-c3c(C(F)(F)F)cc4c(N5CC6CCC(C5)N6)nc(OCC5(CO)CCC5)nc4c3F)ccc(F)c2s1] | A |
| E155 | 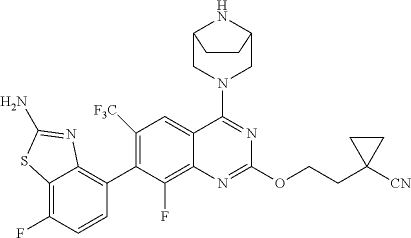 [SMILES: N#CC1(CCOc2nc(N3CC4CCC(C3)N4)c3cc(C(F)(F)F)c(-c4ccc(F)c5sc(N)nc45)c(F)c3n2)CC1] | A |
| E156 | 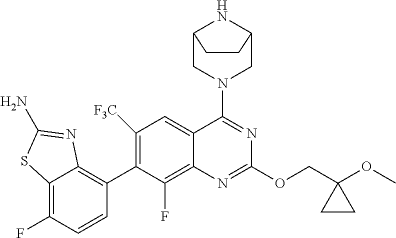 [SMILES: COC1(COc2nc(N3CC4CCC(C3)N4)c3cc(C(F)(F)F)c(-c4ccc(F)c5sc(N)nc45)c(F)c3n2)CC1] | A |
| E157 | 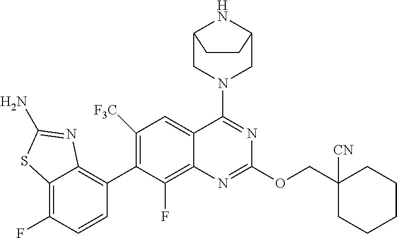 [SMILES: N#CC1(COc2nc(N3CC4CCC(C3)N4)c3cc(C(F)(F)F)c(-c4ccc(F)c5sc(N)nc45)c(F)c3n2)CCCCC1] | A |
| E158 | 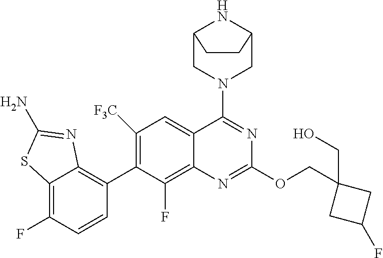 [SMILES: Nc1nc2c(-c3c(C(F)(F)F)cc4c(N5CC6CCC(C5)N6)nc(OCC5(CO)CC(F)C5)nc4c3F)ccc(F)c2s1] | A |
| E159 | 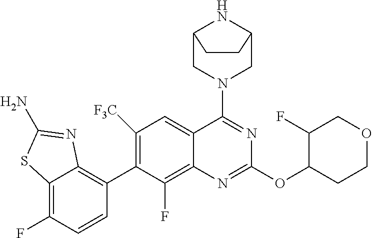 [SMILES: Nc1nc2c(-c3c(C(F)(F)F)cc4c(N5CC6CCC(C5)N6)nc(OC5CCOCC5F)nc4c3F)ccc(F)c2s1] | A |
| E160 | 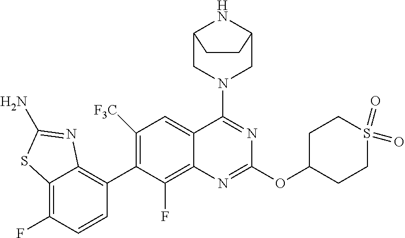 [SMILES: Nc1nc2c(-c3c(C(F)(F)F)cc4c(N5CC6CCC(C5)N6)nc(OC5CCS(=O)(=O)CC5)nc4c3F)ccc(F)c2s1] | A |
| E161 | 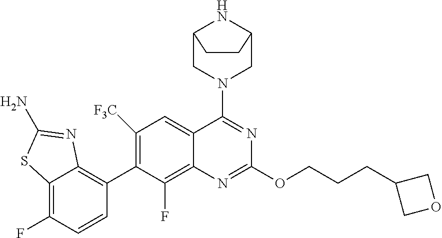 [SMILES: Nc1nc2c(-c3c(C(F)(F)F)cc4c(N5CC6CCC(C5)N6)nc(OCCCC5COC5)nc4c3F)ccc(F)c2s1] | A |
| E162 | 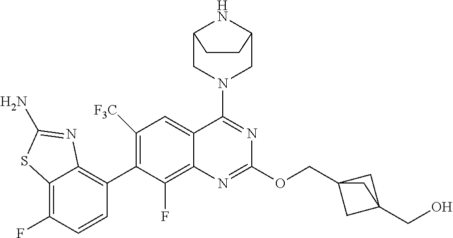 [SMILES: Nc1nc2c(-c3c(C(F)(F)F)cc4c(N5CC6CCC(C5)N6)nc(OCC56CC(CO)(C5)C6)nc4c3F)ccc(F)c2s1] | A |
| E163 | 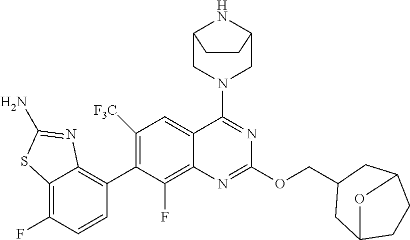 [SMILES: Nc1nc2c(-c3c(C(F)(F)F)cc4c(N5CC6CCC(C5)N6)nc(OCC5CC6CCC(C5)O6)nc4c3F)ccc(F)c2s1] | A |
| E164 | 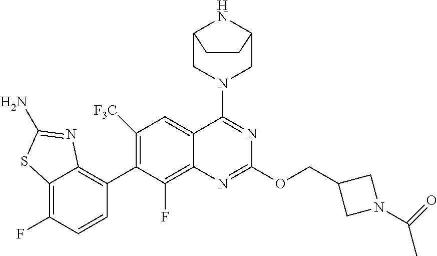 [SMILES: CC(=O)N1CC(COc2nc(N3CC4CCC(C3)N4)c3cc(C(F)(F)F)c(-c4ccc(F)c5sc(N)nc45)c(F)c3n2)C1] | A |
| E165 | 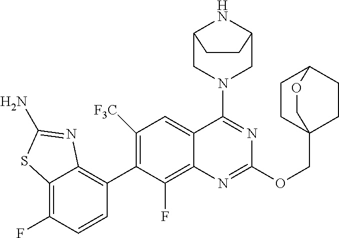 [SMILES: Nc1nc2c(-c3c(C(F)(F)F)cc4c(N5CC6CCC(C5)N6)nc(OCC56CCC(CC5)OC6)nc4c3F)ccc(F)c2s1] | A |
| E166 | 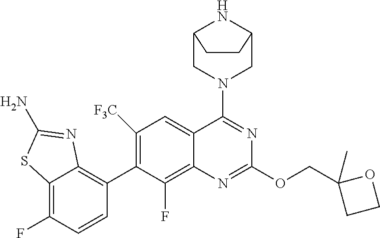 [SMILES: CC1(COc2nc(N3CC4CCC(C3)N4)c3cc(C(F)(F)F)c(-c4ccc(F)c5sc(N)nc45)c(F)c3n2)CCO1] | A |
| E167 | 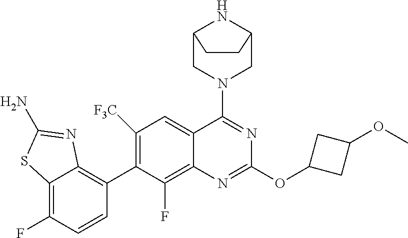 [SMILES: COC1CC(Oc2nc(N3CC4CCC(C3)N4)c3cc(C(F)(F)F)c(-c4ccc(F)c5sc(N)nc45)c(F)c3n2)C1] | B |
| E168 | 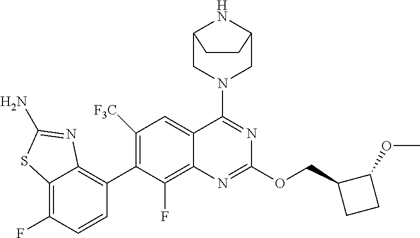 [SMILES: CO[C@@H]1CC[C@H]1COc1nc(N2CC3CCC(C2)N3)c2cc(C(F)(F)F)c(-c3ccc(F)c4sc(N)nc34)c(F)c2n1] | A |
| E169 | 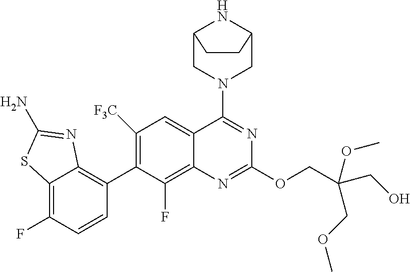 [SMILES: COCC(CO)(COc1nc(N2CC3CCC(C2)N3)c2cc(C(F)(F)F)c(-c3ccc(F)c4sc(N)nc34)c(F)c2n1)OC] | A |
| E170 | 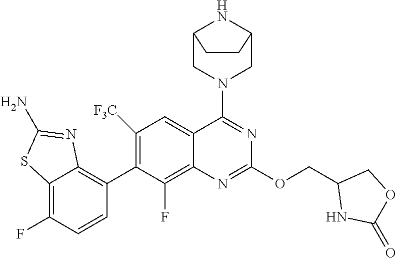 [SMILES: Nc1nc2c(-c3c(C(F)(F)F)cc4c(N5CC6CCC(C5)N6)nc(OCC5COC(=O)N5)nc4c3F)ccc(F)c2s1] | A |
| E171 | 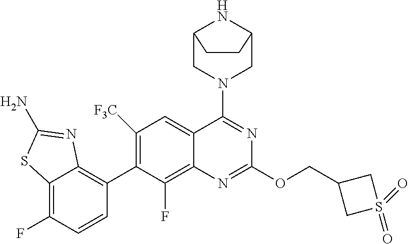 [SMILES: Nc1nc2c(-c3c(C(F)(F)F)cc4c(N5CC6CCC(C5)N6)nc(OCC5CS(=O)(=O)C5)nc4c3F)ccc(F)c2s1] | A |
| E172 | 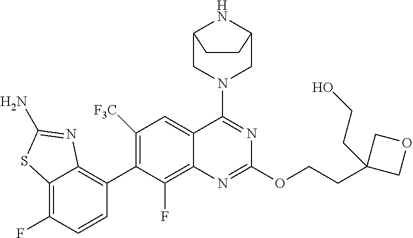 [SMILES: Nc1nc2c(-c3c(C(F)(F)F)cc4c(N5CC6CCC(C5)N6)nc(OCCC5(CCO)COC5)nc4c3F)ccc(F)c2s1] | A |
| E173 | 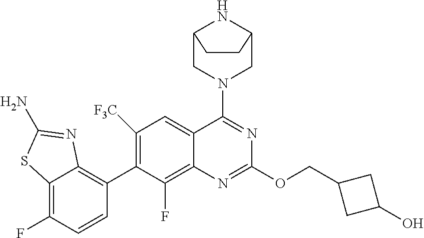 [SMILES: Nc1nc2c(-c3c(C(F)(F)F)cc4c(N5CC6CCC(C5)N6)nc(OCC5CC(O)C5)nc4c3F)ccc(F)c2s1] | A |
| E174 | 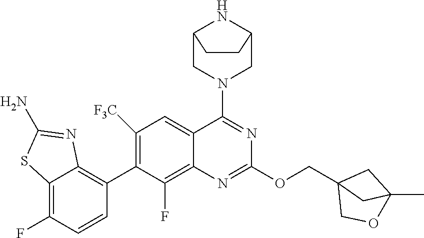 [SMILES: CC12CC(COc3nc(N4CC5CCC(C4)N5)c4cc(C(F)(F)F)c(-c5ccc(F)c6sc(N)nc56)c(F)c4n3)(CO1)C2] | A |
| E175 | 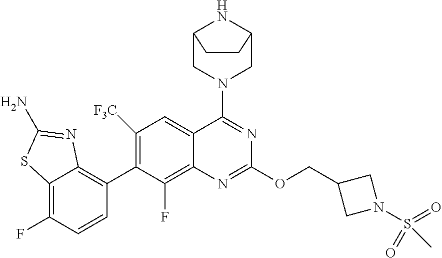 [SMILES: CS(=O)(=O)N1CC(COc2nc(N3CC4CCC(C3)N4)c3cc(C(F)(F)F)c(-c4ccc(F)c5sc(N)nc45)c(F)c3n2)C1] | A |
| E176 | 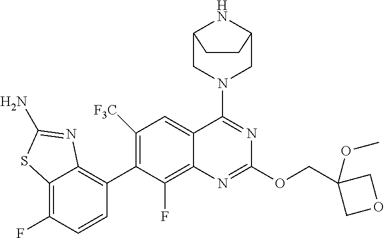 [SMILES: COC1(COc2nc(N3CC4CCC(C3)N4)c3cc(C(F)(F)F)c(-c4ccc(F)c5sc(N)nc45)c(F)c3n2)COC1] | A |
| E177 | 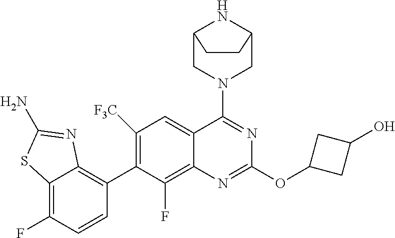 [SMILES: Nc1nc2c(-c3c(C(F)(F)F)cc4c(N5CC6CCC(C5)N6)nc(OC5CC(O)C5)nc4c3F)ccc(F)c2s1] | A |
| E178 | 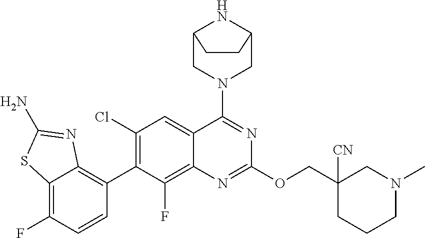 [SMILES: CN1CCCC(C#N)(COc2nc(N3CC4CCC(C3)N4)c3cc(Cl)c(-c4ccc(F)c5sc(N)nc45)c(F)c3n2)C1] | A |
| E179 | 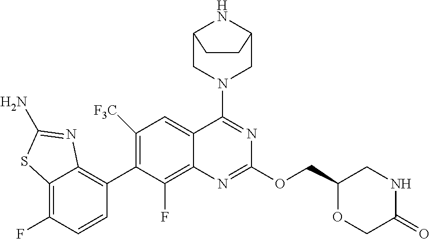 [SMILES: Nc1nc2c(-c3c(C(F)(F)F)cc4c(N5CC6CCC(C5)N6)nc(OC[C@H]5CNC(=O)CO5)nc4c3F)ccc(F)c2s1] | A |
| E180 | 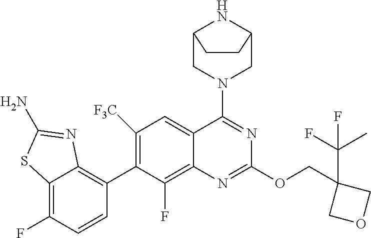 [SMILES: CC(F)(F)C1(COc2nc(N3CC4CCC(C3)N4)c3cc(C(F)(F)F)c(-c4ccc(F)c5sc(N)nc45)c(F)c3n2)COC1] | A |
| E181 | 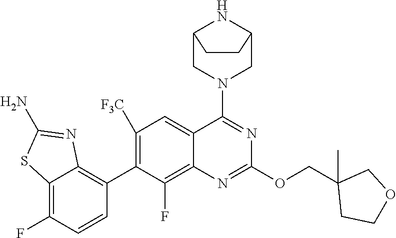 [SMILES: CC1(COc2nc(N3CC4CCC(C3)N4)c3cc(C(F)(F)F)c(-c4ccc(F)c5sc(N)nc45)c(F)c3n2)CCOC1] | A |
| E182 | 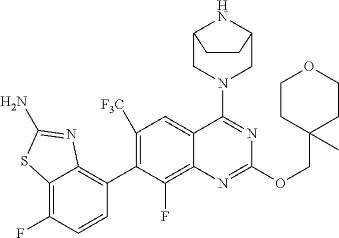 [SMILES: CC1(COc2nc(N3CC4CCC(C3)N4)c3cc(C(F)(F)F)c(-c4ccc(F)c5sc(N)nc45)c(F)c3n2)CCOCC1] | A |
| E183 | 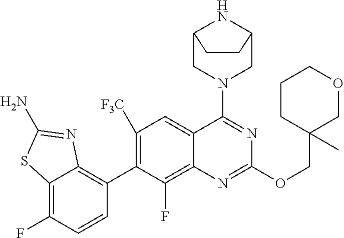 [SMILES: CC1(COc2nc(N3CC4CCC(C3)N4)c3cc(C(F)(F)F)c(-c4ccc(F)c5sc(N)nc45)c(F)c3n2)CCCOC1] | A |
| E184 | 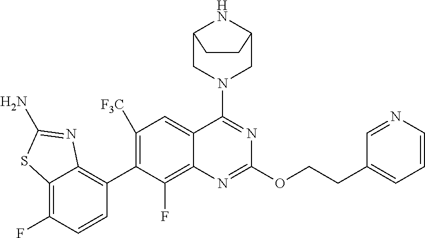 [SMILES: Nc1nc2c(-c3c(C(F)(F)F)cc4c(N5CC6CCC(C5)N6)nc(OCCc5cccnc5)nc4c3F)ccc(F)c2s1] | A |
| E185 | 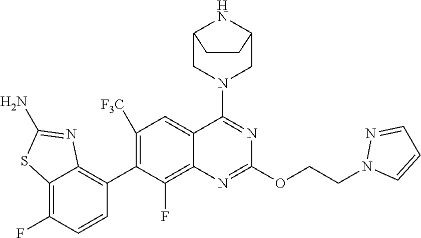 [SMILES: Nc1nc2c(-c3c(C(F)(F)F)cc4c(N5CC6CCC(C5)N6)nc(OCCn5cccn5)nc4c3F)ccc(F)c2s1] | A |
| E186 | 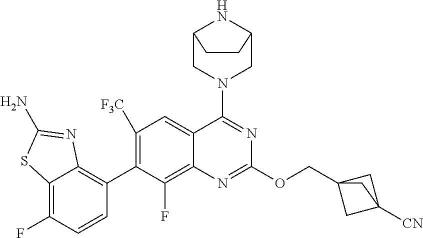 [SMILES: N#CC12CC(COc3nc(N4CC5CCC(C4)N5)c4cc(C(F)(F)F)c(-c5ccc(F)c6sc(N)nc56)c(F)c4n3)(C1)C2] | A |
| E187 | 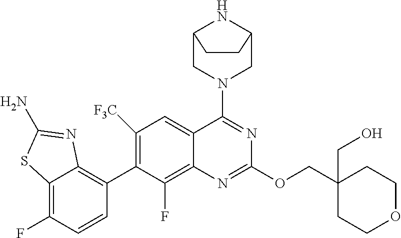 [SMILES: Nc1nc2c(-c3c(C(F)(F)F)cc4c(N5CC6CCC(C5)N6)nc(OCC5(CO)CCOCC5)nc4c3F)ccc(F)c2s1] | A |
| E188 | 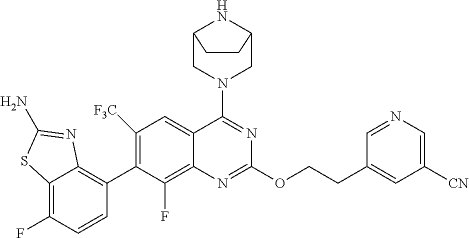 [SMILES: N#Cc1cncc(CCOc2nc(N3CC4CCC(C3)N4)c3cc(C(F)(F)F)c(-c4ccc(F)c5sc(N)nc45)c(F)c3n2)c1] | A |
| E189 | 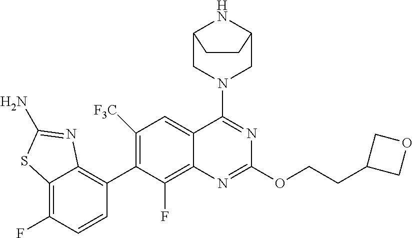 [SMILES: Nc1nc2c(-c3c(C(F)(F)F)cc4c(N5CC6CCC(C5)N6)nc(OCCC5COC5)nc4c3F)ccc(F)c2s1] | A |
| E190 | 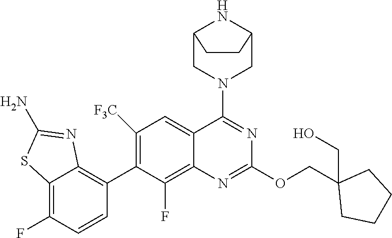 [SMILES: Nc1nc2c(-c3c(C(F)(F)F)cc4c(N5CC6CCC(C5)N6)nc(OCC5(CO)CCCC5)nc4c3F)ccc(F)c2s1] | A |
| E191 | 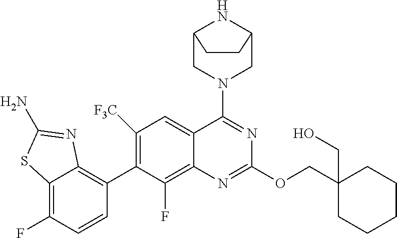 [SMILES: Nc1nc2c(-c3c(C(F)(F)F)cc4c(N5CC6CCC(C5)N6)nc(OCC5(CO)CCCCC5)nc4c3F)ccc(F)c2s1] | A |
| E192 | 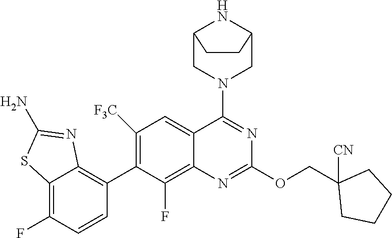 [SMILES: N#CC1(COc2nc(N3CC4CCC(C3)N4)c3cc(C(F)(F)F)c(-c4ccc(F)c5sc(N)nc45)c(F)c3n2)CCCC1] | A |
| E193 | 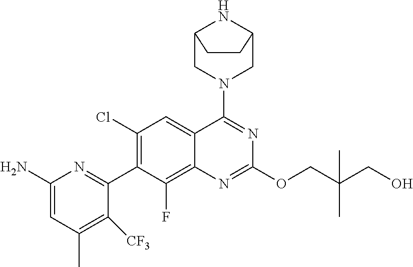 [SMILES: Cc1cc(N)nc(-c2c(Cl)cc3c(N4CC5CCC(C4)N5)nc(OCC(C)(C)CO)nc3c2F)c1C(F)(F)F] | A |
| E194 | 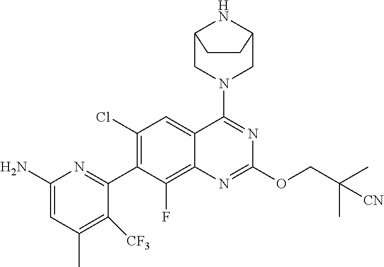 [SMILES: Cc1cc(N)nc(-c2c(Cl)cc3c(N4CC5CCC(C4)N5)nc(OCC(C)(C)C#N)nc3c2F)c1C(F)(F)F] | A |
| E195 | 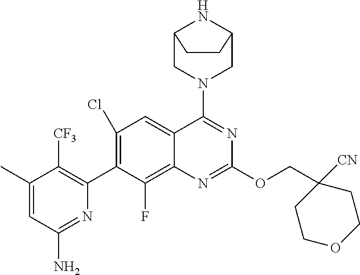 [SMILES: Cc1cc(N)nc(-c2c(Cl)cc3c(N4CC5CCC(C4)N5)nc(OCC4(C#N)CCOCC4)nc3c2F)c1C(F)(F)F] | A |
| E196 | 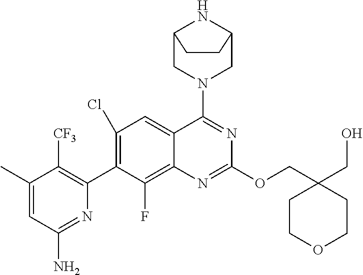 [SMILES: Cc1cc(N)nc(-c2c(Cl)cc3c(N4CC5CCC(C4)N5)nc(OCC4(CO)CCOCC4)nc3c2F)c1C(F)(F)F] | A |
| E197 | 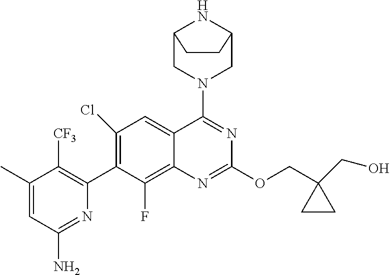 [SMILES: Cc1cc(N)nc(-c2c(Cl)cc3c(N4CC5CCC(C4)N5)nc(OCC4(CO)CC4)nc3c2F)c1C(F)(F)F] | A |
| E198 | 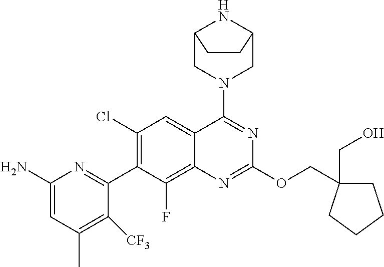 [SMILES: Cc1cc(N)nc(-c2c(Cl)cc3c(N4CC5CCC(C4)N5)nc(OCC4(CO)CCCC4)nc3c2F)c1C(F)(F)F] | B |
| E199 | 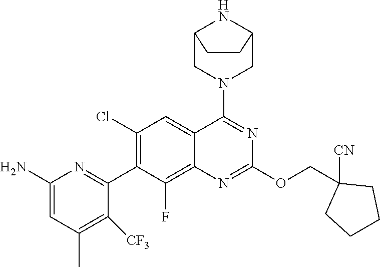 [SMILES: Cc1cc(N)nc(-c2c(Cl)cc3c(N4CC5CCC(C4)N5)nc(OCC4(C#N)CCCC4)nc3c2F)c1C(F)(F)F] | B |
| E200 | 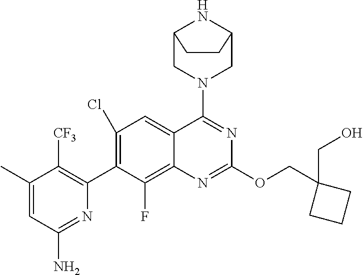 [SMILES: Cc1cc(N)nc(-c2c(Cl)cc3c(N4CC5CCC(C4)N5)nc(OCC4(CO)CCC4)nc3c2F)c1C(F)(F)F] | A |
| E201 | 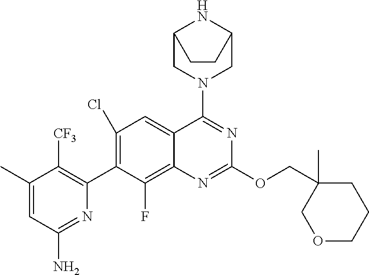 [SMILES: Cc1cc(N)nc(-c2c(Cl)cc3c(N4CC5CCC(C4)N5)nc(OCC4(C)CCCOC4)nc3c2F)c1C(F)(F)F] | C |
| E202 | 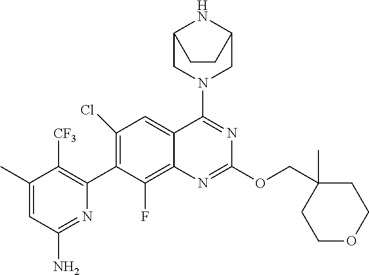 [SMILES: Cc1cc(N)nc(-c2c(Cl)cc3c(N4CC5CCC(C4)N5)nc(OCC4(C)CCOCC4)nc3c2F)c1C(F)(F)F] | B |
| E203 | 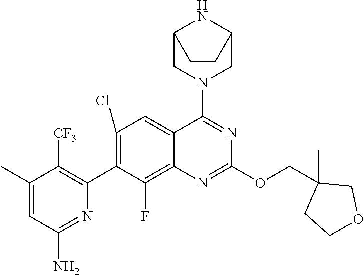 [SMILES: Cc1cc(N)nc(-c2c(Cl)cc3c(N4CC5CCC(C4)N5)nc(OCC4(C)CCOC4)nc3c2F)c1C(F)(F)F] | B |
| E204 | 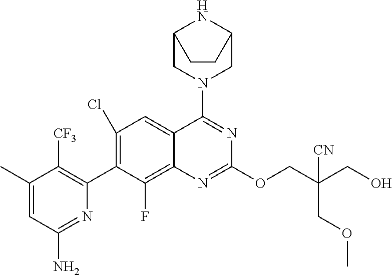 [SMILES: COCC(C#N)(CO)COc1nc(N2CC3CCC(C2)N3)c2cc(Cl)c(-c3nc(N)cc(C)c3C(F)(F)F)c(F)c2n1] | A |
| E205 | 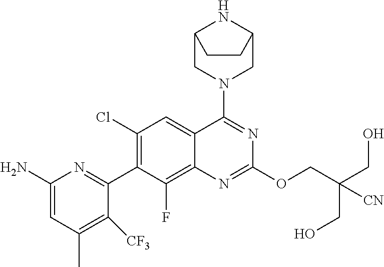 [SMILES: Cc1cc(N)nc(-c2c(Cl)cc3c(N4CC5CCC(C4)N5)nc(OCC(C#N)(CO)CO)nc3c2F)c1C(F)(F)F] | A |
| E206 | 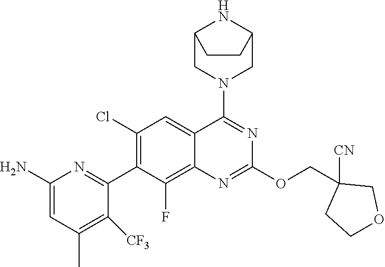 [SMILES: Cc1cc(N)nc(-c2c(Cl)cc3c(N4CC5CCC(C4)N5)nc(OCC4(C#N)CCOC4)nc3c2F)c1C(F)(F)F] | A |
| E207 | 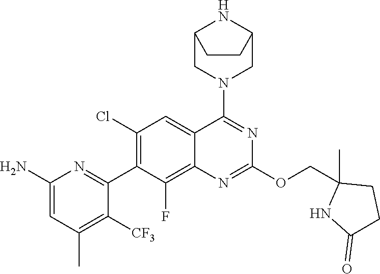 [SMILES: Cc1cc(N)nc(-c2c(Cl)cc3c(N4CC5CCC(C4)N5)nc(OCC4(C)CCC(=O)N4)nc3c2F)c1C(F)(F)F] | A |
| E208 | 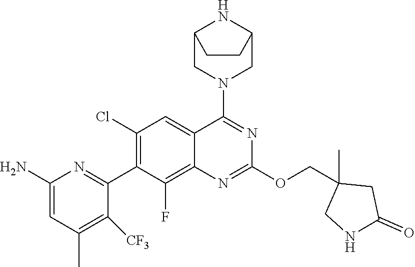 [SMILES: Cc1cc(N)nc(-c2c(Cl)cc3c(N4CC5CCC(C4)N5)nc(OCC4(C)CNC(=O)C4)nc3c2F)c1C(F)(F)F] | A |
| E209 | 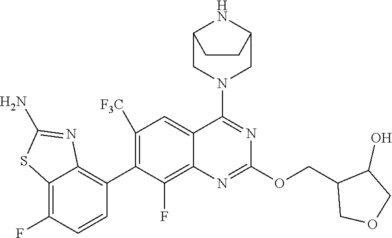 [SMILES: Nc1nc2c(-c3c(C(F)(F)F)cc4c(N5CC6CCC(C5)N6)nc(OCC5COCC5O)nc4c3F)ccc(F)c2s1] | A |
| E210 | 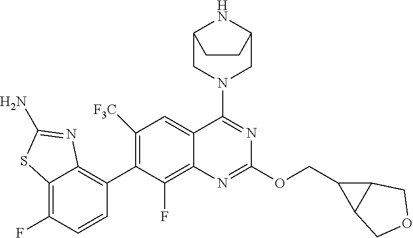 [SMILES: Nc1nc2c(-c3c(C(F)(F)F)cc4c(N5CC6CCC(C5)N6)nc(OCC5C6COCC65)nc4c3F)ccc(F)c2s1] | A |
| E211 | 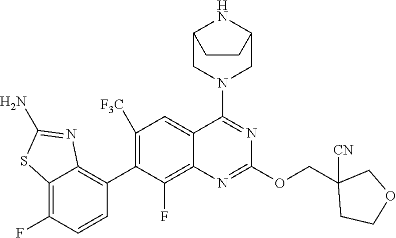 [SMILES: N#CC1(COc2nc(N3CC4CCC(C3)N4)c3cc(C(F)(F)F)c(-c4ccc(F)c5sc(N)nc45)c(F)c3n2)CCOC1] | A |
| E212 | 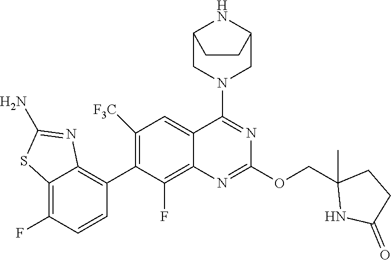 [SMILES: CC1(COc2nc(N3CC4CCC(C3)N4)c3cc(C(F)(F)F)c(-c4ccc(F)c5sc(N)nc45)c(F)c3n2)CCC(=O)N1] | A |
| E213 | 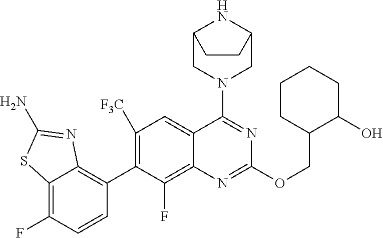 [SMILES: Nc1nc2c(-c3c(C(F)(F)F)cc4c(N5CC6CCC(C5)N6)nc(OCC5CCCCC5O)nc4c3F)ccc(F)c2s1] | A |
| E214 | 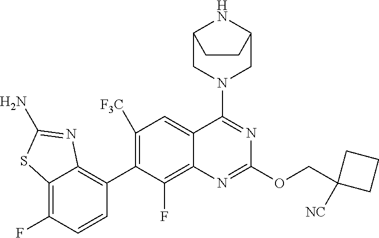 [SMILES: N#CC1(COc2nc(N3CC4CCC(C3)N4)c3cc(C(F)(F)F)c(-c4ccc(F)c5sc(N)nc45)c(F)c3n2)CCC1] | A |
| E215 | 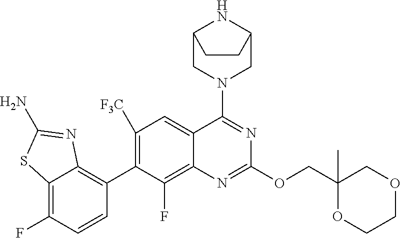 [SMILES: CC1(COc2nc(N3CC4CCC(C3)N4)c3cc(C(F)(F)F)c(-c4ccc(F)c5sc(N)nc45)c(F)c3n2)COCCO1] | A |
| E216 | 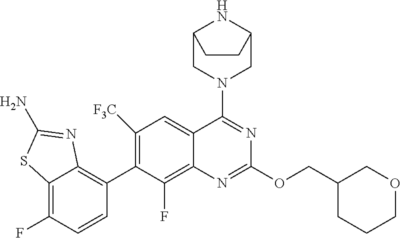 [SMILES: Nc1nc2c(-c3c(C(F)(F)F)cc4c(N5CC6CCC(C5)N6)nc(OCC5CCCOC5)nc4c3F)ccc(F)c2s1] | A |
| E217 | 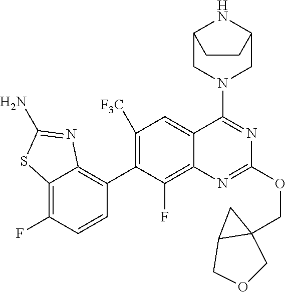 [SMILES: Nc1nc2c(-c3c(C(F)(F)F)cc4c(N5CC6CCC(C5)N6)nc(OCC56COCC5C6)nc4c3F)ccc(F)c2s1] | A |
| E218 | 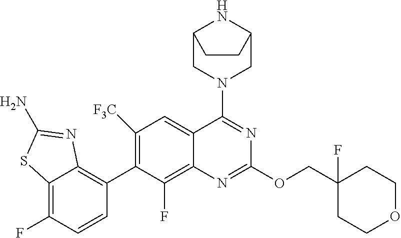 [SMILES: Nc1nc2c(-c3c(C(F)(F)F)cc4c(N5CC6CCC(C5)N6)nc(OCC5(F)CCOCC5)nc4c3F)ccc(F)c2s1] | A |
| E219 | 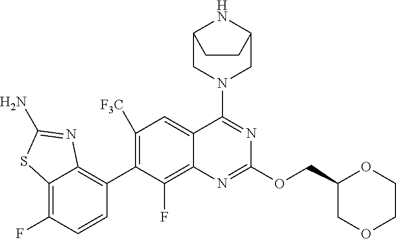 [SMILES: Nc1nc2c(-c3c(C(F)(F)F)cc4c(N5CC6CCC(C5)N6)nc(OC[C@@H]5COCCO5)nc4c3F)ccc(F)c2s1] | A |
| E220 | 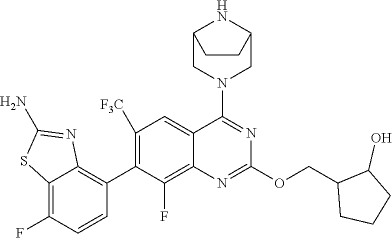 [SMILES: Nc1nc2c(-c3c(C(F)(F)F)cc4c(N5CC6CCC(C5)N6)nc(OCC5CCCC5O)nc4c3F)ccc(F)c2s1] | B |
| E221 | 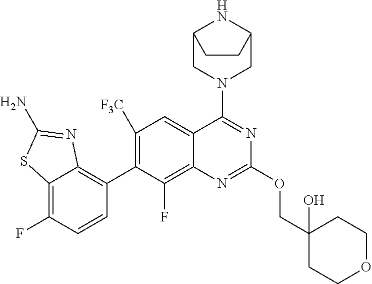 [SMILES: Nc1nc2c(-c3c(C(F)(F)F)cc4c(N5CC6CCC(C5)N6)nc(OCC5(O)CCOCC5)nc4c3F)ccc(F)c2s1] | A |
| E222 | 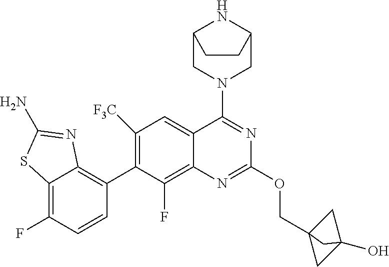 [SMILES: Nc1nc2c(-c3c(C(F)(F)F)cc4c(N5CC6CCC(C5)N6)nc(OCC56CC(O)(C5)C6)nc4c3F)ccc(F)c2s1] | A |
| E223 | 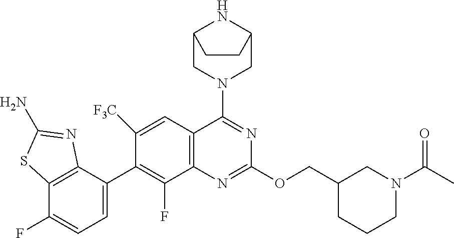 [SMILES: CC(=O)N1CCCC(COc2nc(N3CC4CCC(C3)N4)c3cc(C(F)(F)F)c(-c4ccc(F)c5sc(N)nc45)c(F)c3n2)C1] | A |
| E224 | 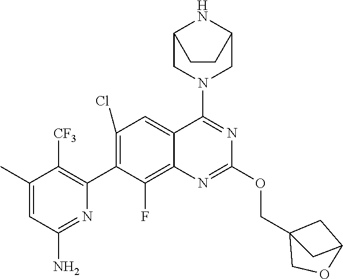 [SMILES: Cc1cc(N)nc(-c2c(Cl)cc3c(N4CC5CCC(C4)N5)nc(OCC45COC(C4)C5)nc3c2F)c1C(F)(F)F] | A |
| E225 | 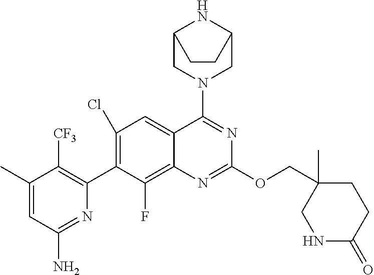 [SMILES: Cc1cc(N)nc(-c2c(Cl)cc3c(N4CC5CCC(C4)N5)nc(OCC4(C)CCC(=O)NC4)nc3c2F)c1C(F)(F)F] | A |
| E226 | 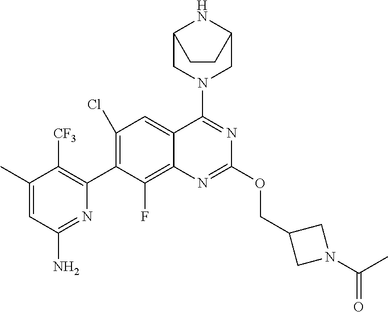 [SMILES: CC(=O)N1CC(COc2nc(N3CC4CCC(C3)N4)c3cc(Cl)c(-c4nc(N)cc(C)c4C(F)(F)F)c(F)c3n2)C1] | A |
| E227 | 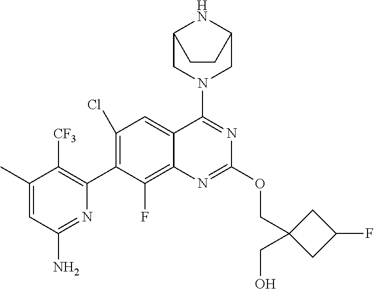 [SMILES: Cc1cc(N)nc(-c2c(Cl)cc3c(N4CC5CCC(C4)N5)nc(OCC4(CO)CC(F)C4)nc3c2F)c1C(F)(F)F] | A |
| E228 | 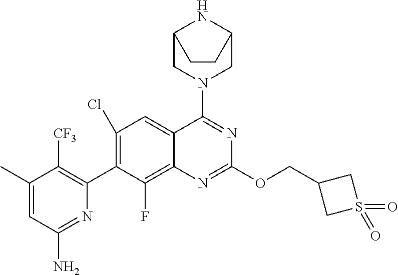 [SMILES: Cc1cc(N)nc(-c2c(Cl)cc3c(N4CC5CCC(C4)N5)nc(OCC4CS(=O)(=O)C4)nc3c2F)c1C(F)(F)F] | A |
| E229 | 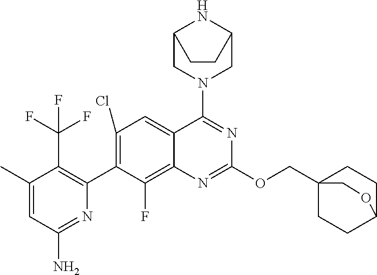 [SMILES: Cc1cc(N)nc(-c2c(Cl)cc3c(N4CC5CCC(C4)N5)nc(OCC45CCC(CC4)OC5)nc3c2F)c1C(F)(F)F] | A |
| E230 | 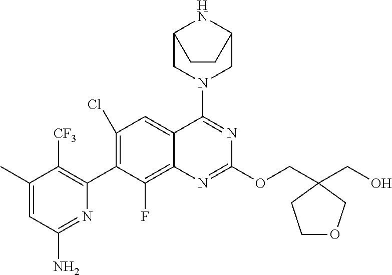 [SMILES: Cc1cc(N)nc(-c2c(Cl)cc3c(N4CC5CCC(C4)N5)nc(OCC4(CO)CCOC4)nc3c2F)c1C(F)(F)F] | A |
| E231 | 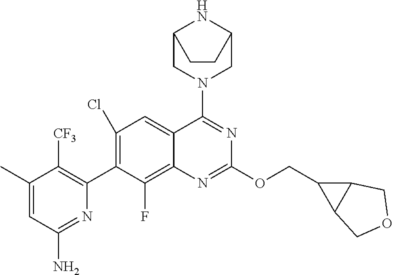 [SMILES: Cc1cc(N)nc(-c2c(Cl)cc3c(N4CC5CCC(C4)N5)nc(OCC4C5COCC54)nc3c2F)c1C(F)(F)F] | C |
| E232 | 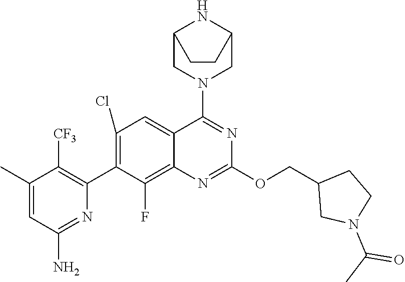 [SMILES: CC(=O)N1CCC(COc2nc(N3CC4CCC(C3)N4)c3cc(Cl)c(-c4nc(N)cc(C)c4C(F)(F)F)c(F)c3n2)C1] | A |
| E233 | 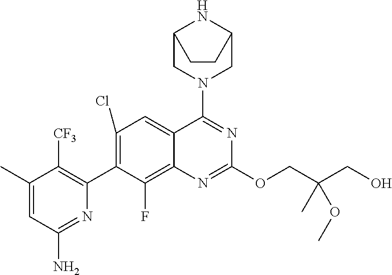 [SMILES: COC(C)(CO)COc1nc(N2CC3CCC(C2)N3)c2cc(Cl)c(-c3nc(N)cc(C)c3C(F)(F)F)c(F)c2n1] | A |
| E234 | 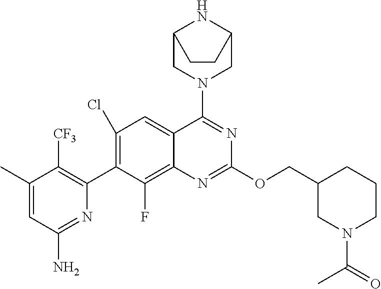 [SMILES: CC(=O)N1CCCC(COc2nc(N3CC4CCC(C3)N4)c3cc(Cl)c(-c4nc(N)cc(C)c4C(F)(F)F)c(F)c3n2)C1] | B |
| E235 | 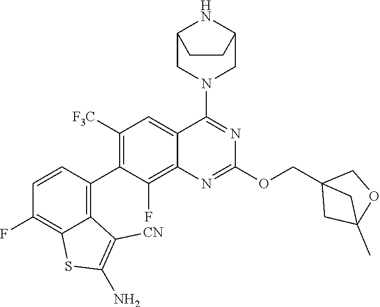 [SMILES: CC12CC(COc3nc(N4CC5CCC(C4)N5)c4cc(C(F)(F)F)c(-c5ccc(F)c6sc(N)c(C#N)c56)c(F)c4n3)(CO1)C2] | A |
| E236 | 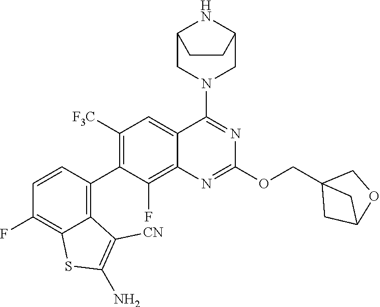 [SMILES: N#Cc1c(N)sc2c(F)ccc(-c3c(C(F)(F)F)cc4c(N5CC6CCC(C5)N6)nc(OCC56COC(C5)C6)nc4c3F)c12] | A |
| E237 | 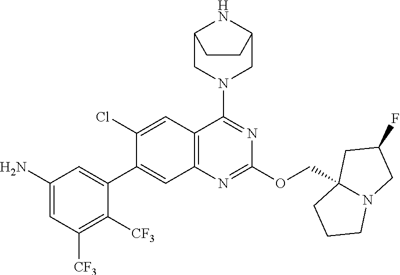 [SMILES: Nc1cc(-c2cc3nc(OC[C@@]45CCCN4C[C@H](F)C5)nc(N4CC5CCC(C4)N5)c3cc2Cl)c(C(F)(F)F)c(C(F)(F)F)c1] | A |

It is to be understood that, if any prior art publication is referred to herein; such reference does not constitute an admission that the publication forms a part of the common general knowledge in the art in any country.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1. A bridged compound of Formula (I): 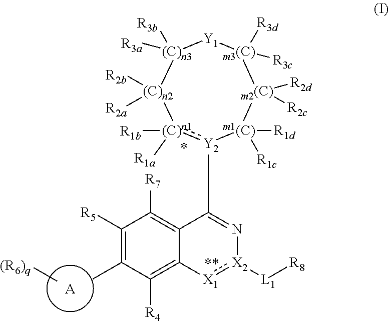
or a pharmaceutically acceptable salt thereof, or a stereoisomer thereof,
wherein:
ring A is an aryl group or a 5- to 7-membered monocyclic heteroaryl or 8- to 12-membered bicyclic heteroaryl group; [symbol]
and [symbol] are each independently a single bond or double bond;
Y₁ is —NH— or —C(R_{Y1a})(NHR_{Y1b})—;
Y₂ is C and R_{1b} is absent in the case that [symbol] is a double bond; or Y₂ is N or CR_{Y2} in the case that [symbol] is a single bond;
X₁ is N, CH or CO; X₂ is C or N;
provided that when [symbol] is a double bond X₁ is N or CH, X₂ is C; or
when [symbol] is a single bond, X₁ is CO, X₂ is N;
n1, n2, n3, m1, m2, and m3 are each independently 0 or 1, provided that at least one of n1, n2 and n3 is 1;
and at least one of m1, m2 and m3 is 1;
q is 0, 1, 2, 3, 4, 5, or 6;
R₁ₐ, R_{1b}, R₂ₐ, R_{2b}, R₃ₐ, R_{3b}, R_{1c}, R_{1d}, R_{2c}, R₂ₐ, R_{3c}, R₃ₐ, R_{Y1a}, R_{Y1b} and R_{Y2}, if present, are each independently hydrogen, halogen, —C₁₋₈-alkyl, C₃-C₈cycloalkyl, 3- to 8-membered heterocyclyl, C₆-C₁₂aryl, 5- to 12-membered heteroaryl, —CN, —OR₁ₑ, or —NR₁ₑR_{1f}, ; each of said —C₁₋₈alkyl, C₃-C₈cycloalkyl, 3- to 8-membered heterocyclyl, C₆-C₁₂aryl or 5- to 12-membered heteroaryl is optionally substituted with at least one substituent R_{1g}; and
at least one pair of (R₁ₐ and R_{1c}), (R₁ₐ and R_{2c}), (R₁ₐ and R_{3c}), (R₂ₐ and R_{1c}), (R₂ₐ and R_{2c}), (R₂ₐ and R_{3c}), (R₃ₐ and R_{1c}), (R₃ₐ and R_{2c}), (R₃ₐ and R_{3c}), (R_{Y1a} and R_{Y2}), (R_{Y1a} and R₁ₐ), (R_{Y1a} and R₂ₐ), (R_{Y1a} and R_{1c}), (R_{Y1a} and R_{2c}), (R_{Y1b} and R₁ₐ), (R_{Y1b} and R₂ₐ), (R_{Y1b} and R_{1c}), (R_{Y1b} and R_{2c}), and (R_{Y1b} and R_{Y2}) form a bridge containing one, two, three, or four —CH₂— moieties in addition to the two bridgeheads, wherein one of the —CH₂— moiety is optionally replaced with —O—, —S— or —NH— and wherein said bridge is optionally substituted with at least one substituent R_{1g};
optionally, (R_{Y1a} and R_{Y1b}), (R_{Y1a} and R₃ₐ), (R_{Y1a} and R_{3c}), (R_{Y1b} and R₃ₐ), or (R_{Y1b} and R_{3c}) form 3- to 12-membered ring, the said ring comprises 0-3 heteroatoms selected from nitrogen, sulfur and oxygen and the said bridge is optionally substituted with at least one substituent R_{1g};
R₁ₑ and R_{1f} are each independently selected from hydrogen, —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl;
R_{1g}, at each occurrence, is independently halogen, —C₁₋₈-alkyl, —C₂₋₈alkenyl, —C₂₋₈alkynyl, —C₃-C₈cycloalkyl, 3- to 8-membered heterocyclyl, —C₆-C₁₂aryl, 5- to 12-membered heteroaryl, —C₁₋₈haloalkyl, C₁₋₈alkoxy-C₁₋₈alkyl-, —CN, —OH, —NH₂, or —C₁₋₈alkoxyl;
R₆ is hydrogen, halogen, —C₁₋₈-alkyl, —C₂₋₈alkenyl, —C₂₋₈alkynyl, C₃-C₈cycloalkyl, 3- to 8-membered heterocyclyl, C₆-C₁₂aryl, 5- to 12-membered heteroaryl, —CN, oxo, —OR₆ₐ, —SR₆ₐ, —SO₂R₆ₐ, —SO₂NR₆ₐR_{6b}, —COR₆ₐ, —CO₂R₆ₐ, —CONR₆ₐR_{6b}, —NR₆ₐR_{6b}, —NR₆ₐCOR_{6b}, —NR₆ₐCO₂R_{6b}, or —NR₆ₐSO₂R_{6b}; each of said —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl is optionally substituted with at least one substituent R_{6c}; or
two R₆ together with the atoms to which they are attached, form a 5, 6, 7 or 8-membered unsaturated or saturated ring, said ring comprising 0-3 heteroatoms independently selected from nitrogen, oxygen or sulfur; said ring is optionally substituted with at least one substituent R_{6d};
R₆ₐ and R_{6b} are each independently selected from hydrogen, —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl, wherein each of said —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl is optionally substituted with at least one substituent R_{6d};
R_{6c}, at each occurrence, is independently halogen, —C₁₋₈-alkyl, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R_{6d} is hydrogen, halogen, —C₁₋₈-alkyl, —C₂₋₈alkenyl, —C₂₋₈alkynyl, C₃-C₈cycloalkyl, 3- to 8-membered heterocyclyl, C₆-C₁₂aryl, 5- to 12-membered heteroaryl, —CN, oxo, —OR₆ₑ, —SO₂R₆ₑ, —SO₂NR₆ₑR_{6f}, —COR₆ₑ, —O₂R₆ₑ, —CONR₆ₑR_{6f}, —NR₆ₑR_{6f}, —NR₆ₑCOR_{6f}, —NR₆ₑCO₂R_{6f}, or —NR₆ₑSO₂R_{6f}; each of said —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl is optionally substituted with at least one substituent R_{6g};
R₆ₑ and R_{6f} are each independently selected from hydrogen, —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl, each of said —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl is optionally substituted with at least one substituent R_{6g};
R_{6g}, at each occurrence, is independently halogen, —C₁₋₈-alkyl, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R₄, R₅, and R₇ are each independently hydrogen, halogen, —C₁₋₈-alkyl, C₃-C₈cycloalkyl, —C₂₋₈alkenyl, —C₂₋₈alkynyl, 3- to 8-membered heterocyclyl, C₆-C₁₂aryl, 5- to 12-membered heteroaryl, —CN, oxo, —OR₄ₐ, —SR₄ₐ, —SO₂R₄ₐ, —SO₂NR₄ₐR_{4b}, —COR₄ₐ, —CO₂R₄ₐ, —CONR₄ₐR_{4b}, —NR₄ₐR_{4b}, —NR₄ₐCOR_{4b}, —NR₄ₐCO₂R_{4b}, or —NR₄ₐSO₂R_{4b}; wherein each of said —C₁₋₈alkyl, C₃-C₈cycloalkyl, —C₂₋₈alkenyl, —C₂₋₈alkynyl, 3- to 8-membered heterocyclyl, C₆-C₁₂aryl or 5- to 12-membered heteroaryl is optionally substituted with at least one substituent R_{4c},
R₄ₐ and R_{4b} are each independently selected from hydrogen, —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl; wherein each of said —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl is optionally substituted with at least one substituent R_{4d};
R_{4c} and R_{4d}, at each occurrence, are each independently halogen, —C₁₋₈alkyl, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
L₁ is selected from a single bond, —O—, —S—, —NR^{L1a}—, —C(O)—, —C₁₋₈alkylene-, *^{L1}—O—C₁₋₈alkylene-**^{L1}, *^{L1}—C₁₋₈alkylene-O—**^{L1}, *^{L1}—S—C₁₋₈alkylene-**^{L1}, *^{L1}—C₁₋₈alkylene-S—**^{L1}, —C₃₋₈cycloalkylene-, *^{L1}—O—C₃-C₈cycloalkylene-**^{L1}, *^{L1}—C₃-C₈cycloalkylene-O—**^{L1}, *^{L1}—S—C₃-C₈cycloalkylene-**^{L1}, *^{L1}—C₃-C₈cycloalkylene-S—**^{L1}, *^{L1}—O—C₁₋₈alkylene-NR^{L1a}—**^{L1}, *^{L1}—NR^{L1a}—C₁₋₈alkylene-O—**^{L1}, *^{L1}—S—C₁₋₈alkylene-NR^{L1a}—**^{L1}, *^{L1}—NR^{L1a}—C₁₋₈alkylene-S—**^{L1}, *^{L1}—O—C₁₋₈alkylene-C(O)—**^{L1}, *^{L1}—C(O)—C₁₋₈alkylene-O—**^{L1}, *^{L1}—S—C₁₋₈alkylene-C(O)—**^{L1}, *^{L1}—C(O)—C₁₋₈alkylene-S—**^{L1}, *^{L1}—C(O)—C₁₋₈alkylene-**^{L1}, *^{L1}—C₁₋₈alkylene-C(O)—**^{L1}, *^{L1}—NR^{L1a}—C₁₋₈alkylene-**^{L1}, *^{L1}—C₁₋₈alkylene-NR^{L1a}—**^{L1}, *^{L1}—C(O)NR^{L1a}—C₁₋₈alkylene-**^{L1}, *^{L1}—C₁₋₈alkylene-C(O)NR^{L1a}—**^{L1}, *^{L1}—NR^{L1a}C(O)—C₁₋₈alkylene-**^{L1}, *^{L1}—C₁₋₈alkylene-NR^{L1a}C(O)—**^{L1}, *^{L1}—C(O)O—C₁₋₈alkylene-**^{L1}, *^{L1}—C₁₋₈alkylene-C(O)O—**^{L1}, *^{L1}—OC(O)—C₁₋₈alkylene-**^{L1}, *^{L1}—C₁₋₈alkylene-OC(O)—**^{L1}, 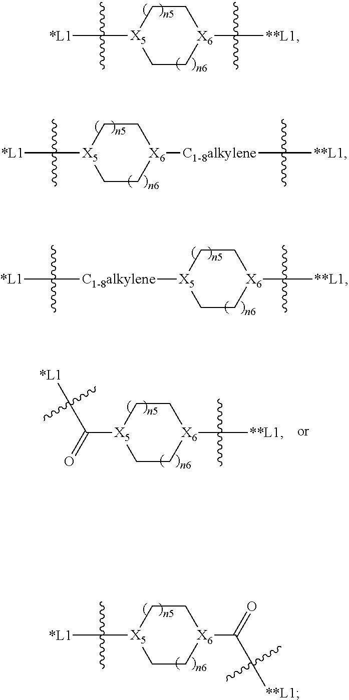
each of said single bond, —C₁₋₈alkylene-, *^{L1}—O—C₁₋₈alkylene-**^{L1}, *^{L1}—C₁₋₈alkylene-O—**^{L1}, *^{L1}—S—C₁₋₈alkylene-**^{L1}, *^{L1}—C₁₋₈alkylene-S—**^{L1}, —C₃-C₈cycloalkylene-, *^{L1}—O—C₃-C₈cycloalkylene-**^{L1}, *^{L1}—C₃-C₈cycloalkylene-O—**^{L1}, *^{L1}—S—C₃-C₈cycloalkylene-**^{L1}, *^{L1}—C₃-C₈cycloalkylene-S—**^{L1}, *^{L1}—O—C₁₋₈alkylene-NR^{L1a}—**^{L1}, *^{L1}—NR^{L1a}—C₁₋₈alkylene-O—**^{L1}, *^{L1}—S—C₁₋₈alkylene-NR^{L1a}—**^{L1}, *^{L1}—NR^{L1a}—C₁₋₈alkylene-S—**^{L1}, *^{L1}—O—C₁₋₈alkylene-C(O)—**^{L1}, *^{L1}—C(O)—C₁₋₈alkylene-O—**^{L1}, *^{L1}—S—C₁₋₈alkylene-C(O)—**^{L1}, *^{L1}—C(O)—C₁₋₈alkylene-S—**^{L1}, *^{L1}—C(O)—C₁₋₈alkylene-**^{L1}, *^{L1}—C₁₋₈alkylene-C(O)—**^{L1}, *^{L1}—NR^{L1a}—C₁₋₈alkylene-**^{L1}, *^{L1}—C₁₋₈alkylene-NR^{L1a}—**^{L1}, *^{L1}—C(O)NR^{L1a}—C₁₋₈alkylene-**^{L1}, *^{L1}—C₁₋₈alkylene-C(O)NR^{L1a}—**^{L1}, *^{L1}—NR^{L1a}C(O)—C₁₋₈alkylene-**^{L1}, *^{L1}—C₁₋₈alkylene-NR^{L1a}C(O)—**^{L1}, *^{L1}—C(O)O—C₁₋₈alkylene-**^{L1}, *^{L1}—C₁₋₈alkylene-C(O)O—**^{L1}, *^{L1}—OC(O)—C₁₋₈alkylene-**^{L1}, *^{L1}—C₁₋₈alkylene-OC(O)—**^{L1}, 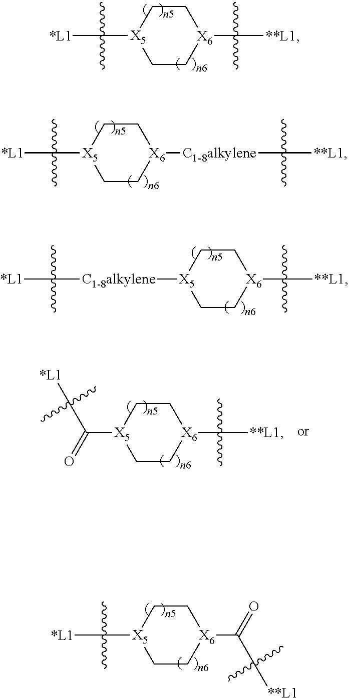
is optionally substituted with at least one R^{L1b};
wherein **^{L1} refers to the position attached to the 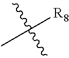
moiety, and *^{L1} refers to the position attached to the other side;
R^{L1a} is selected from hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl, each of said —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl is optionally substituted with at least one substituent R^{L1c};
each of said R^{L1b} and R^{L1c} is independently halogen, hydroxy, —C₁₋₈alkyl, —C₁₋₈alkoxy, —C₂₋₈alkenyl, —C₂₋₈alkynyl, C₃-C₈cycloalkyl, 3- to 8-membered heterocyclyl, C₆-C₁₂aryl or 5- to 12-membered heteroaryl; or
two R^{L1b} or two R^{L1c} together with the atoms to which they are attached, form a 3- to 6-membered unsaturated or saturated ring, said ring comprising 0-3 heteroatoms independently selected from nitrogen, oxygen or sulfur; said ring is optionally substituted with at least one substituent halogen, hydroxy, —C₁₋₈alkyl, —C₁₋₈alkoxy, —C₂₋₈alkenyl, —C₂₋₈alkynyl, C₃-C₈cycloalkyl, 3- to 8-membered heterocyclyl, C₆-C₁₂aryl or 5- to 12-membered heteroaryl;
each of X₅ and X₆ is CH or N;
n5 and n6 are each independently 0, 1 or 2;
R₈ is hydrogen, halogen, —C₁₋₈-alkyl, C₃-C₈cycloalkyl, 3- to 12-membered heterocyclyl, C₆-C₁₂aryl, 5- to 12-membered heteroaryl, oxo, —CN, —OR₃ₐ, —COR₈ₐ, —CO₂R₈ₐ, —CONR₈ₐR_{8b}, —NR₈ₐR_{8b}, —NR₈ₐCOR_{8b} or —NR₈ₐCO₂R_{8b}, wherein each of said —C₁₋₈alkyl, C₃-C₈cycloalkyl, 3- to 12-membered heterocyclyl, C₆-C₁₂aryl or 5- to 12-membered heteroaryl is optionally substituted with at least one substituent R_{8c};
R₈ₐ and R_{8b} are each independently selected from hydrogen, —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl, 5- to 12-membered heteroaryl or oxo; wherein each of said —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl, 5- to 12-membered heteroaryl is optionally substituted with at least one substituent R_{8d}; or
R₈ₐ and R_{8b} together with the carbon atoms to which they are attached, form a 3- to 8-membered unsaturated or saturated ring, said ring comprising 0-3 heteroatoms independently selected from nitrogen, oxygen or sulfur; said ring is optionally substituted with at least one substituent R_{8c};
R_{8c}, at each occurrence, is independently halogen, —C₁₋₈-alkyl, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl, oxo, —CN, —OR₈ₑ, —COR₈ₑ, —CO₂R₈ₑ, —CONR₈ₑR_{8f}, —NR₈ₑR_{8f}, —NR₈ₑCOR_{8f} or —NR₈ₑCO₂R_{8f}, wherein each of said —C₁₋₈alkyl, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl is optionally substituted with at least one substituent R_{8d};
two R_{8c} together with the carbon atoms to which they are attached, form a 3- to 8-membered unsaturated or saturated ring, said ring comprising 0-3 heteroatoms independently selected from nitrogen, oxygen or sulfur; said ring is optionally substituted with at least one substituent R_{8d}; or two R_{8c} together with the atoms to which they are attached form a bridge containing one, two, three, or four —CH2- moieties in addition to the two bridgeheads, wherein one of the —CH₂— moiety is optionally replaced with —O—, —S— or —NH—;
R_{8d} is hydrogen, halogen, —C₁₋₈-alkyl, C₃-C₈cycloalkyl, 3- to 8-membered heterocyclyl, C₆-C₁₂aryl, 5- to 12-membered heteroaryl, oxo, —CN, —OR_{8g}, —COR_{8g}, —CO₂R_{8g}, —CONR_{8g}R₈ₕ, —NR_{8g}R₈ₕ, —NR_{8g}COR₈ₕ or —NR_{8g}CO₂R₈ₕ, wherein each of said —C₁₋₈alkyl, C₃-C₈cycloalkyl, 3- to 8-membered heterocyclyl, C₆-C₁₂aryl or 5- to 12-membered heteroaryl is optionally substituted with at least one substituent R₈ᵢ;
R₈ₑ, R_{8f}, R_{8g}, R₈ₕ and R₈ᵢ are each independently selected from hydrogen, —C₁₋₈alkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl.

2. The compound of claim 1, wherein Y₁ is —NH— or —C(R_{Y1a})(NH₂)—.

3. The compound ofany one claim 1, wherein R₁ₐ, R_{1b}, R₂ₐ, R_{2b}, R₃ₐ, R_{3b}, R_{1c}, R_{1d}, R_{2c}, R_{2d}, R_{3c}, R₃ₐ, R_{Y1a}, R_{Y1b} and R_{Y2}, if present, are each independently hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —CN, —OR₁ₑ or —NR₁ₑR_{1f}; each of said methyl, ethyl, propyl, butyl, pentyl, hexyl, heptyl, octyl, cyclopropyl, cyclobutyl, cyclopentyl, cyclohexyl, cycloheptyl or cyclooctyl is optionally substituted with at least one substituent R_{1g},
one pair of (R₁ₐ and R_{1c}), (R₁ₐ and R_{2c}), (R₁ₐ and R_{3c}), (R₂ₐ and R_{1c}), (R₂ₐ and R_{2c}), (R₂ₐ and R_{3c}), (R₃ₐ and R_{1c}), (R₃ₐ and R_{2c}), (R₃ₐ and R_{3c}), (R_{Y1a} and R_{Y2}), and (R_{Y1b} and R_{Y2}) form a bridge containing one, two, three, or four —CH₂— moieties in addition to the two bridgeheads, wherein one of the —CH₂— moiety is optionally replaced with —O—, —S— or —NH— and wherein said bridge is optionally substituted with at least one substituent R_{1g};
R₁ₑ and R_{1f} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, —C₂₋₈alkenyl, —C₂₋₈alkynyl, C₁₋₈alkoxy-C₁₋₈alkyl-, 3- to 8-membered heterocyclyl, phenyl or 5- to 12-membered heteroaryl;
R_{1g}, at each occurrence, is independently —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, phenyl, or 5- to 12-membered heteroaryl.

4. The compound of claim 1, wherein the ring 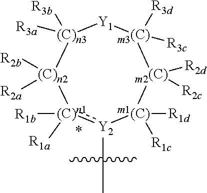
is a bridged bicyclic ring.

5. The compound of claim 1, wherein, the ring 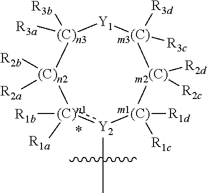
is selected from:
Y₁ is NH, Y₂ is N, [symbol] is a single bond, R₃ₐ and R_{3c} form a bridge containing one, two, or three —CH₂— moieties in addition to the two bridgeheads (nitrogen), wherein one of the —CH₂— moiety is optionally replaced with —O—, —S— or —NH— and wherein said bridge is optionally substituted with halogen or alkyl, and n3=m3=n1=m1=1 and n2=m2=0; or
Y₁ is NH, Y₂ is N, [symbol] is a single bond, R₂ₐ and R_{2c} form a bridge containing one, two, or three —CH₂— moieties in addition to the two bridgeheads (nitrogen), wherein one of the —CH₂— moiety is optionally replaced with —O—, —S— or —NH—, and n1=m1=n2=m2=n3=m3=1; or
Y₁ is NH, Y₂ is N, [symbol] is a single bond, R₁ₐ and R_{1c} form a bridge containing one, two, or three —CH₂— moieties in addition to the two bridgeheads (nitrogen), wherein one of the —CH₂— moiety is optionally replaced with —O—, —S— or —NH—, and n3=m3=n1=m1=1 and n2=m2=0; or
Y₁ is NH, Y₂ is N, [symbol] is a single bond, R₁ₐ and R_{1c} form a bridge containing one, two, or three —CH₂— moieties in addition to the two bridgeheads (nitrogen), wherein one of the —CH₂— moiety is optionally replaced with —O—, —S— or —NH—, and n2=m2=n3=m3=0 and n1=m1=1; or
Y₁ is NH, Y₂ is CH, [symbol] is a single bond, or Y₂ is C, [symbol] a double bond, R₂ₐ and R_{1c} form a bridge containing one, two, or three —CH₂— moieties in addition to the two bridgeheads (one nitrogen and one carbon atom), wherein one of the —CH₂— moiety is optionally replaced with —O—, —S— or —NH—, and n1=n2=n3=m1=1, and m2=m3=0; or
Y₁ is NH, Y₂ is CH, [symbol] is a single bond, or Y₂ is C, [symbol] is a double bond, R₃ₐ and R_{3c} form abridge containing one, two, or three —CH₂— moieties in addition to the two bridgeheads (one nitrogen and one carbon atom), wherein one of the —CH₂— moiety is optionally replaced with —O—, —S— or —NH—, and n1=m1=n3=m3=1, and n2=m2=0 or n3=m3=m1=1, and n1=n2=m2=0; or
Y₁ is CH, Y₂ is CH, [symbol] is a single bond, R_{Y1a} and R_{Y2} form a bridge containing one, two, or three —CH₂— moieties in addition to the two bridgeheads (two carbon atoms), wherein one of the —CH₂— moiety is optionally replaced with —O—, —S— or —NH—, and n1=m1=n3=m3=0, and n2=m2=1; or
Y₁ is NH, Y₂ is N, [symbol] is a single bond, R₃ₐ and R_{1c} form a bridge containing one, two, or three —CH₂— moieties in addition to the two bridgeheads (nitrogen), wherein one of the —CH₂— moiety is optionally replaced with —O—, —S— or —NH—, n1=m1=n3=m3=1, and n2=m2=0; or
Y₁ is NH, Y₂ is —CH(NH₂), [symbol] is a single bond, R₁ₐ and R_{1c} form a bridge containing one, two, or three —CH₂— moieties in addition to the two bridgeheads (one nitrogen and one carbon atom), wherein one of the —CH₂— moiety is optionally replaced with —O—, —S— or —NH—, n1=m1=n3=1, and n2=m2=m3=0.

6. The compound of claim 1, wherein the 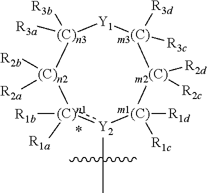
moiety is selected from 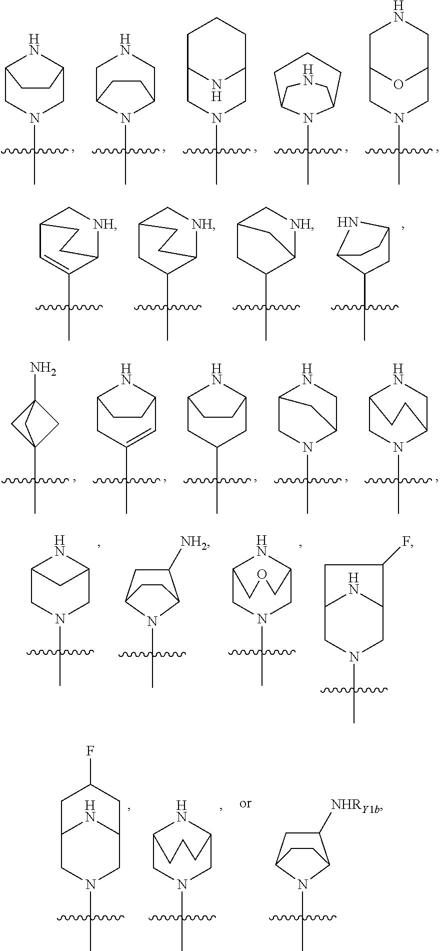
wherein R_{Y1b} is —C₁₋₅alkyl and is optionally substituted with fluoro or alkoxyl.

7. The compound of claim 1, wherein [symbol] is a double bond, X₁ is N, X₂ is C; or [symbol] is a single bond, X₁ is C═O, and X₂ is N.

8. The compound of claim 1, wherein R₆ is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl, hexynyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, —CN, oxo, —OR₆ₐ, —SR₆ₐ or —NR₆ₐR_{6b}; each of said methyl, ethyl, propyl, butyl, pentyl, hexyl, heptyl, octyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl, hexynyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with at least one substituent R_{6c};
R₆ₐ and R_{6b} are each independently selected from hydrogen, methyl, ethyl, propyl, butyl, pentyl, hexyl, heptyl, octyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl; wherein each of said methyl, ethyl, propyl, butyl, pentyl, hexyl, heptyl, octyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R_{6d};
R_{6c}, at each occurrence, is independently —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl; or
two R₆ together with the atoms to which they are attached, form a 5- or 6-membered unsaturated (preferred aromatic) or saturated ring, said ring comprising 0, 1, 2, or 3 heteroatoms independently selected from nitrogen, oxygen or sulfur; said ring is optionally substituted with at least one substituent R_{6d};
R_{6d} is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CN, oxo, —OR₆ₑ or —NR₆ₑR_{6f}; each of said methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R_{6g};
R₆ₑ and R_{6f} are each independently selected from hydrogen, methyl, ethyl, propyl, butyl, pentyl, hexyl, heptyl, octyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl;
R_{6g}, at each occurrence, is independently —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, phenyl, or 5- to 12-membered heteroaryl.

9. The compound of claim 1, wherein R₄, R₅ and R₇ are each independently hydrogen, —F, —Cl, —Br, —I, methyl, ethyl, propyl, butyl, pentyl, hexyl, heptyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl, hexynyl, —CN, oxo, —OR₄ₐ, —SR₄ₐ or —NR₄ₐR_{4b}; each of said methyl, ethyl, propyl, butyl, pentyl, hexyl, heptyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl or hexynyl is optionally substituted with at least one substituent R_{4c},
R₄ₐ and R_{4b} are each independently selected from hydrogen, methyl, ethyl, propyl, butyl, pentyl, hexyl, heptyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl, hexynyl; wherein each of said methyl, ethyl, propyl, butyl, pentyl, hexyl, heptyl, cyclopropyl, cyclobutyl, cyclopentyl, cyclohexyl, cycloheptyl, cyclooctyl, vinyl, propylenyl, allyl, butenyl, butadienyl, pentenyl, pentadienyl, hexenyl, hexadienyl, ethynyl, propynyl, butynyl, pentynyl or hexynyl is optionally substituted with at least one substituent R₄ₐ;
R_{4c} and R_{4d}, at each occurrence, are each independently —F, —Cl, —Br, —I, methyl, ethyl, propyl, butyl, pentyl, hexyl, heptyl, vinyl, propylenyl, allyl, butenyl, butadienyl, pentenyl, pentadienyl, hexenyl, hexadienyl, ethynyl, propynyl, butynyl, pentynyl, hexynyl, cyclopropyl, cyclobutyl, cyclopentyl, cyclohexyl, cycloheptyl, cyclooctyl, 3- to 8-membered heterocyclyl, —C₆-C₁₂aryl, or 5- to 12-membered heteroaryl.

10. The compound of claim 1, wherein R₄, R₅ and R₇ are each independently hydrogen, —F, —Cl, —Br, —I, methyl, ethyl, propyl, iso-propyl, iso-butyl, tert-butyl, —CF₃, —CHF₂, —CH₂F, —OCHF₂, —OCF₃, —CF₂CH₃, —CF═CH, cyclopropyl, cyclobutyl, cyclopentyl, vinyl, propylenyl, allyl, ethynyl, propynyl, —Sme, —SCF₃, —SCHF₂ or —SCH₂F.

11. The compound of claim 1, wherein the 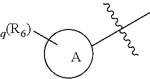
moiety is an aryl group selected from phenyl or naphthyl substituted with one or two R₆.

12. The compound of claim 1, wherein the 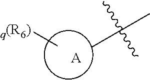
moiety is a 5- to 7-membered monocyclic heteroaryl or 8- to 12-membered bicyclic heteroaryl group substituted with one or two R₆.

13. The compound of claim 1, wherein the 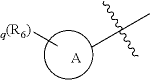
moiety is

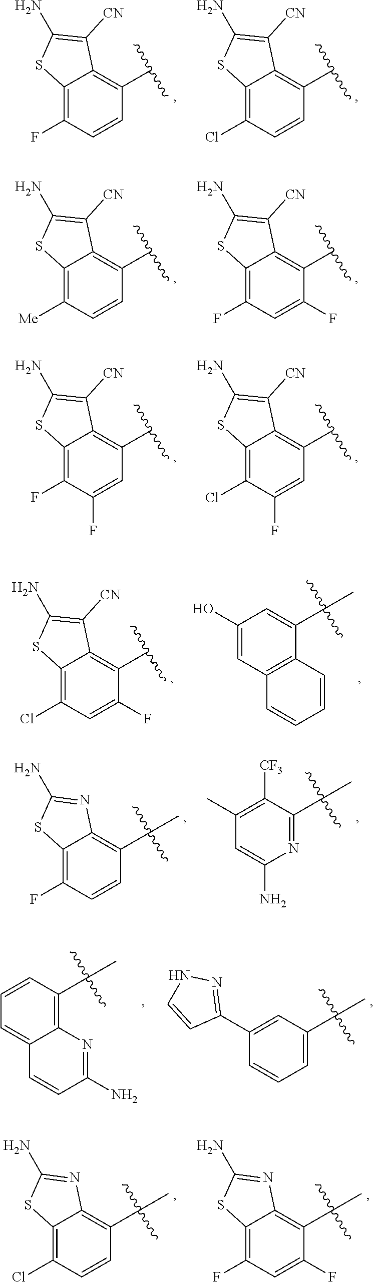

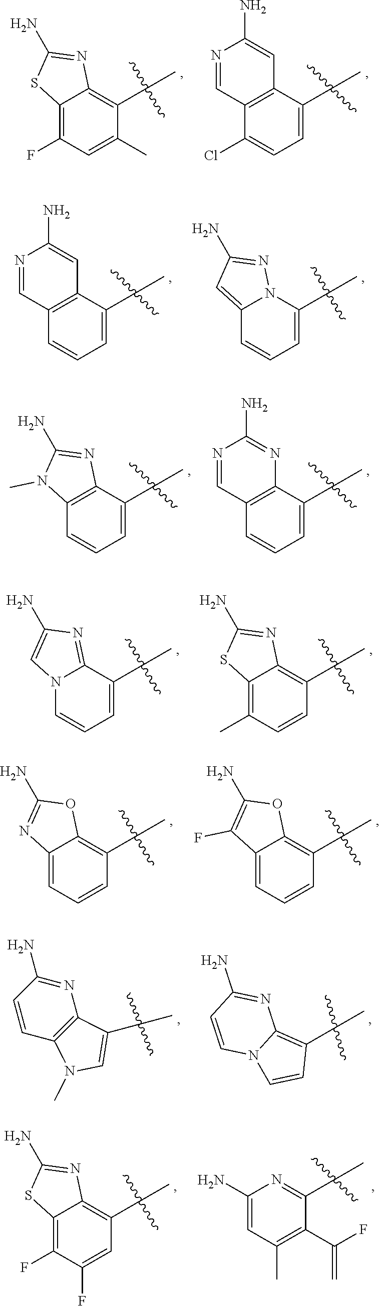

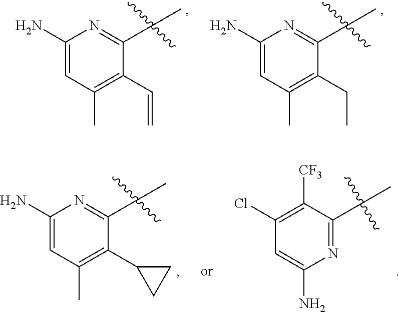 [SMILES: *c1nc(N)cc(C)c1C=C ; *c1nc(N)cc(C)c1CC ; *c1nc(N)cc(C)c1C1CC1 ; *c1nc(N)cc(Cl)c1C(F)(F)F]

14. The compound of claim 1, wherein X₁ is N and X₂ is C.

15. The compound of claim 1, wherein the 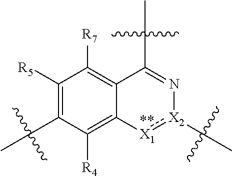
moiety is

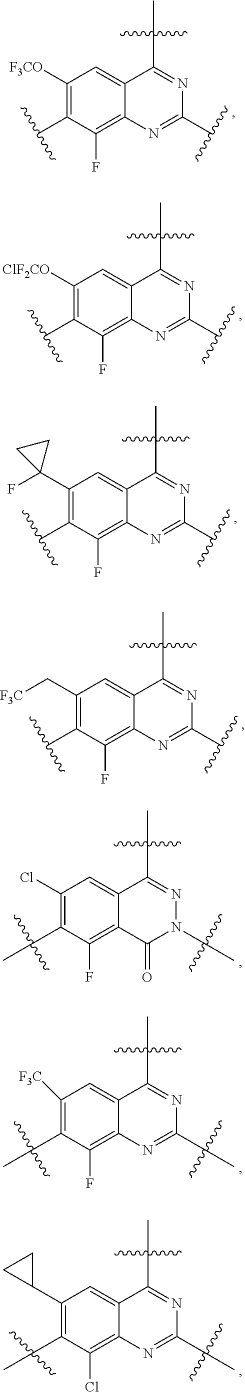

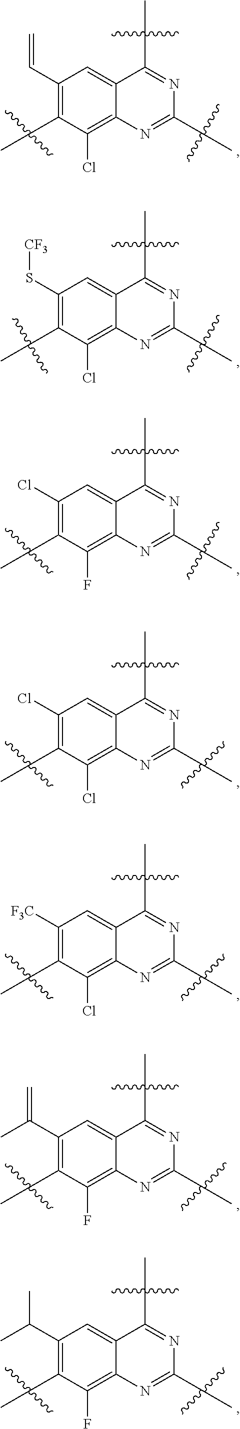

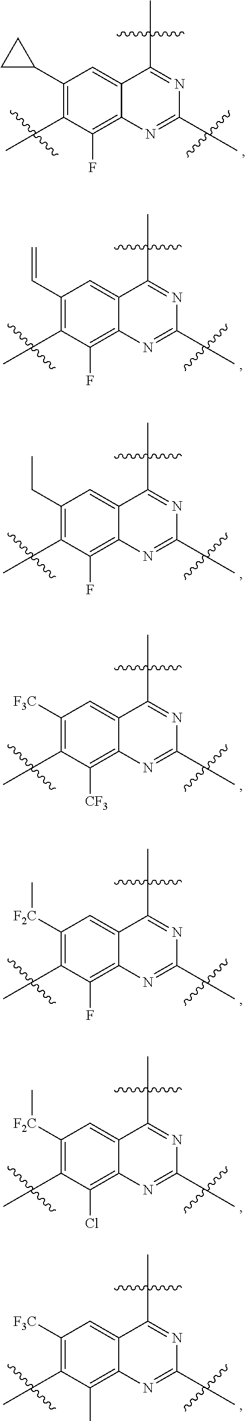

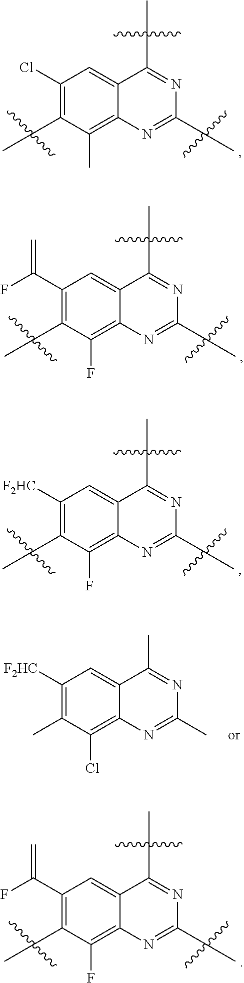

16. The compound of claim 1, wherein L₁ is selected from a single bond, —O—, —S—, —NR^{L1a}—, —C(O)—, —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH₂CH₂CH₂CH₂CH₂CH₂CH₂—, —CH₂CH₂CH₂CH₂CH₂CH₂CH₂CH₂—, —C₃-C₈cycloalkylene-, *^{L1}—CH₂—**^{L1}, *^{L1}—O—CH₂CH₂—**^{L1}, *^{L1}—O—CH₂CH₂CH₂—**^{L1}, *^{L1}—O—CH₂CH₂CH₂CH₂—**^{L1}, *^{L1}—O—CH₂CH₂CH₂CH₂CH₂—**^{L1}, *—O—CH₂CH₂CH₂CH₂CH₂CH₂—**^{L1}, *^{L1}—CH₂CH₂CH₂CH₂CH₂CH₂CH₂—**^{L1}, *^{L1}—O—CH₂CH₂CH₂CH₂CH₂CH₂CH₂CH₂—**^{L1}, *^{L1}—CH₂—O—**^{L1}, *^{L1}—CH₂CH₂—O—**^{L1}, *^{L1}—CH₂CH₂CH₂—O—**^{L1}, *^{L1}—CH₂CH₂CH₂CH₂—O—**^{L1}, *^{L1}—CH₂CH₂CH₂CH₂CH₂—O—**^{L1}, *^{L1}—CH₂CH₂CH₂CH₂CH₂CH₂—O—**^{L1}, *^{L1}—CH₂CH₂CH₂CH₂CH₂CH₂CH₂—O—**^{L1}, *^{L1}—CH₂CH₂CH₂CH₂CH₂CH₂CH₂CH₂—O—**^{L1}, *^{L1}—S—CH₂—**^{L1}, *^{L1}—S—CH₂CH₂—**^{L1}, *^{L1}—S—CH₂CH₂CH₂—**^{L1}, *^{L1}—S—CH₂CH₂CH₂CH₂—**^{L1}, *^{L1}—S—CH₂CH₂CH₂CH₂CH₂—**^{L1}, *^{L1}—S—CH₂CH₂CH₂CH₂CH₂CH₂—**^{L1}, *^{L1}—S—CH₂CH₂CH₂CH₂CH₂CH₂CH₂—**^{L1}, *^{L1}—S—CH₂CH₂CH₂CH₂CH₂CH₂CH₂CH₂—**^{L1}, *^{L1}—CH₂—S—**^{L1}, *^{L1}—CH₂CH₂—S—**^{L1}, *^{L1}—CH₂CH₂CH₂—S—**^{L1}, *^{L1}—CH₂CH₂CH₂CH₂—S—**^{L1}, *^{L1}—CH₂CH₂CH₂CH₂CH₂—S—**^{L1}, *^{L1}—CH₂CH₂CH₂CH₂CH₂CH₂—S—**^{L1}, *^{L1}—CH₂CH₂CH₂CH₂CH₂CH₂CH₂—S—**^{L1}, *^{L1}—CH₂CH₂CH₂CH₂CH₂CH₂CH₂CH₂—S—**^{L1}, *^{L1}—O—C₃-C₈cycloalkylene-**^{L1}, *^{L1}—C₃-C₈cycloalkylene-O—**^{L1}, *^{L1}—S—C₃-C₈cycloalkylene-**^{L1}, *^{L1}—C₃-C₈cycloalkylene-S—**^{L1}, *^{L1}—O—CH₂—NR^{L1a}—**^{L1}, *^{L1}—O—CH₂CH₂—NR^{L1a}—**^{L1}, *^{L1}—O—CH₂CH₂CH₂—NR^{L1a}—**^{L1}, *^{L1}—O—CH₂CH₂CH₂CH₂—NR^{L1a}—**^{L1}, *^{L1}—O—CH₂CH₂CH₂CH₂CH₂—NR^{L1a}—**^{L1}, *^{L1}—O—CH₂CH₂CH₂CH₂CH₂CH₂—NR^{L1a}—**^{L1},*^{L1}—O—CH₂CH₂CH₂CH₂CH₂CH₂CH₂—NR^{L1a}—**^{L1}, *^{L1}—CH₂CH₂CH₂CH₂CH₂CH₂CH₂CH₂—NR^{L1a}—**^{L1}, *^{L1}—NR^{L1a}—CH₂—O—**^{L1}, *^{L1}—NR^{L1a}—CH₂CH₂—O—**^{L1}, *^{L1}—NR^{L1a}—CH₂CH₂CH₂—O—**^{L1}, *^{L1}—NR^{L1a}—CH₂CH₂CH₂CH₂—O—**^{L1}, *^{L1}—NR^{L1a}—CH₂CH₂CH₂CH₂CH₂—O—**^{L1}, *^{L1}—NR^{L1a}—CH₂CH₂CH₂CH₂CH₂CH₂—O—**^{L1}, *^{L1}—NR^{L1a}—CH₂CH₂CH₂CH₂CH₂CH₂CH₂—O—**^{L1}, *^{L1}—NR^{L1a}—CH₂CH₂CH₂CH₂CH₂CH₂CH₂CH₂—O—**^{L1}, *^{L1}—S—CH₂—NR^{L1a}—**^{L1}, *^{L1}—S—CH₂CH₂—NR^{L1a}—**^{L1}, *^{L1}—S—CH₂CH₂CH₂—NR^{L1a}—**^{L1}, *^{L1}—S—CH₂CH₂CH₂CH₂—NR^{L1a}—*^{L1}, *^{L1}—S—CH₂CH₂CH₂CH₂CH₂—NR^{L1a}—**^{L1}, *^{L1}—S—CH₂CH₂CH₂CH₂CH₂CH₂—NR^{L1a}—**^{L1},*^{L1}—S—CH₂CH₂CH₂CH₂CH₂CH₂CH₂—NR^{L1a}—**^{L1}, *^{L1}—S—CH₂CH₂CH₂CH₂CH₂CH₂CH₂CH₂—NR^{L1a}—**^{L1}, *^{L1}—NR^{L1a}—CH₂—S—**^{L1}, *NR^{L1a}—CH₂CH₂—S—**^{L1}, *^{L1}—NR^{L1a}—CH₂CH₂CH₂—S—**^{L1}, *^{L1}—NR^{L1a}—CH₂CH₂CH₂CH₂—S—**^{L1}, *^{L1}—NR^{L1a}—CH₂CH₂CH₂CH₂CH₂—S—**^{L1}, *^{L1}—NR^{L1a}—CH₂CH₂CH₂CH₂CH₂CH₂—S—**^{L1}, *^{L1}—NR^{L1a}—CH₂CH₂CH₂CH₂CH₂CH₂CH₂—S—**^{L1}, *^{L1}—NR^{L1a}—CH₂CH₂CH₂CH₂CH₂CH₂CH₂CH₂—S—**^{L1}, *^{L1}—O—CH₂—CO—**^{L1}, *^{L1}—O—CH₂CH₂—CO—**^{L1}, *^{L1}—O—CH₂CH₂CH₂—CO—**^{L1}, *^{L1}—O—CH₂CH₂CH₂CH₂—CO—**^{L1}, *^{L1}—O—CH₂CH₂CH₂CH₂CH₂—CO—**^{L1}, *^{L1}—O—CH₂CH₂CH₂CH₂CH₂CH₂—CO—**^{L1}, *^{L1}—O—CH₂CH₂CH₂CH₂CH₂CH₂CH₂—CO—**^{L1}, *^{L1}—O—CH₂CH₂CH₂CH₂CH₂CH₂CH₂CH₂—CO—**^{L1}, *^{L1}—CO—CH₂—O—**^{L1}, *^{L1}—CO—CH₂CH₂—O—**^{L1}, *^{L1}—CO—CH₂CH₂CH₂—O—**^{L1}, *^{L1}—CO—CH₂CH₂CH₂CH₂—O—**^{L1}, *^{L1}—CO—CH₂CH₂CH₂CH₂CH₂—O—**^{L1}, *^{L1}—CO—CH₂CH₂CH₂CH₂CH₂CH₂—O—**^{L1}, *^{L1}—CO—CH₂CH₂CH₂CH₂CH₂CH₂CH₂—O—**^{L1}, *^{L1}—CO—CH₂CH₂CH₂CH₂CH₂CH₂CH₂CH₂—O—**^{L1}, *^{L1}—S—CH₂—CO—**^{L1}, *^{L1}—S—CH₂CH₂—CO—**^{L1}, *^{L1}—S—CH₂CH₂CH₂—CO—**^{L1}, *^{L1}—S—CH₂CH₂CH₂CH₂—CO—**^{L1}, *^{L1}—S—CH₂CH₂CH₂CH₂CH₂—CO—**^{L1}, *^{L1}—S—CH₂CH₂CH₂CH₂CH₂CH₂—CO—**^{L1}, *^{L1}—S—CH₂CH₂CH₂CH₂CH₂CH₂CH₂—CO—**^{L1}, *^{L1}—S—CH₂CH₂CH₂CH₂CH₂CH₂CH₂CH₂—CO—**^{L1}, *^{L1}—CO—CH₂—S—**^{L1}, *^{L1}—CO—CH₂CH₂—S—**^{L1}, *^{L1}—CO—CH₂CH₂CH₂—S—**^{L1}, *^{L1}—CO—CH₂CH₂CH₂CH₂—S—**^{L1}, *^{L1}—CO—CH₂CH₂CH₂CH₂CH₂—S—**^{L1}, *^{L1}—CO—CH₂CH₂CH₂CH₂CH₂CH₂—S—**^{L1}, *^{L1}—CO—CH₂CH₂CH₂CH₂CH₂CH₂CH₂—S—**^{L1}, *^{L1}—CO—CH₂CH₂CH₂CH₂CH₂CH₂CH₂CH₂—S—**^{L1}, *^{L1}—C(O)—CH₂—**^{L1}, *^{L1}—C(O)—CH₂CH₂—**^{L1}, *^{L1}—C(O)—CH₂CH₂CH₂—**^{L1}, *^{L1}—C(O)—CH₂CH₂CH₂CH₂—**^{L1}, *^{L1}—C(O)—CH₂CH₂CH₂CH₂CH₂—**^{L1}, *^{L1}—C(O)—CH₂CH₂CH₂CH₂CH₂CH₂—**^{L1}, *^{L1}—C(O)—CH₂CH₂CH₂CH₂CH₂CH₂CH₂—**^{L1}, *^{L1}—C(O)—CH₂CH₂CH₂CH₂CH₂CH₂CH₂CH₂—**^{L1}, *^{L1}—CH₂—C(O)—**^{L1}, *^{L1}—CH₂CH₂—C(O)—**^{L1}, *^{L1}—CH₂CH₂CH₂—C(O)—**^{L1}, *^{L1}—CH₂CH₂CH₂CH₂—C(O)—**^{L1}, *^{L1}—CH₂CH₂CH₂CH₂CH₂—C(O)—**^{L1}, *^{L1}—CH₂CH₂CH₂CH₂CH₂CH₂—C(O)—**^{L1}, *^{L1}—CH₂CH₂CH₂CH₂CH₂CH₂CH₂—C(O)—**^{L1}, *^{L1}—CH₂CH₂CH₂CH₂CH₂CH₂CH₂CH₂—C(O)—**^{L1}, *^{L1}—CH₂—NR^{L1a}—**^{L1}, *^{L1}—CH₂CH₂—NR^{L1a}—**^{L1}, *^{L1}—CH₂CH₂CH₂—NR^{L1a}—**^{L1}, *^{L1}—CH₂CH₂CH₂CH₂—NR^{L1a}—**^{L1}, *^{L1}—CH₂CH₂CH₂CH₂CH₂—NR^{L1a}—**^{L1}, *^{L1}—CH₂CH₂CH₂CH₂CH₂CH₂—NR^{L1a}—**^{L1}, *^{L1}—CH₂CH₂CH₂CH₂CH₂CH₂CH₂—NR^{L1a}—**^{L1}, *^{L1}—CH₂CH₂CH₂CH₂CH₂CH₂CH₂CH₂—NR^{L1a}—**^{L1}, *^{L1}—NR^{L1a}—CH₂—**^{L1}, *^{L1}—NR^{L1a}—CH₂CH₂—**^{L1}, *^{L1}—NR^{L1a}—CH₂CH₂CH₂—**^{L1}, *^{L1}—NR^{L1a}—CH₂CH₂CH₂CH₂—**^{L1}, *^{L1}—NR^{L1a}—CH₂CH₂CH₂CH₂CH₂—**^{L1}, *^{L1}—NR^{L1a}—CH₂CH₂CH₂CH₂CH₂CH₂—**^{L1}, *^{L1}—NR^{L1a}—CH₂CH₂CH₂CH₂CH₂CH₂CH₂—**^{L1}, *^{L1}—NR^{L1a}—CH₂CH₂CH₂CH₂CH₂CH₂CH₂CH₂—**^{L1}, *^{L1}—CH₂—C(O)NR^{L1a}—**^{L1}, *^{L1}—CH₂CH₂—C(O)NR^{L1a}—**^{L1}, *^{L1}—CH₂CH₂CH₂—C(O)NR^{L1a}—**^{L1}, *^{L1}—CH₂CH₂CH₂CH₂—C(O)NR^{L1a}—**^{L1}, *^{L1}—CH₂CH₂CH₂CH₂CH₂—C(O)NR^{L1a}—**^{L1}, *^{L1}—CH₂CH₂CH₂CH₂CH₂CH₂—C(O)NR^{L1a}—**^{L1}, *^{L1}—CH₂CH₂CH₂CH₂CH₂CH₂CH₂—C(O)NR^{L1a}—**^{L1}, *^{L1}—CH₂CH₂CH₂CH₂CH₂CH₂CH₂CH₂—C(O)NR^{L1a}—**^{L1}, *^{L1}—C(O)NR^{L1a}—CH₂—**^{L1}, *^{L1}—C(O)NR^{L1a}—CH₂CH₂—**^{L1}, *^{L1}—C(O)NR^{L1a}—CH₂CH₂CH₂—**^{L1}, *^{L1}—C(O)NR^{L1a}—CH₂CH₂CH₂CH₂—**^{L1}, *^{L1}—C(O)NR^{L1a}—CH₂CH₂CH₂CH₂CH₂—**^{L1}, *^{L1}—C(O)NR^{L1a}—CH₂CH₂CH₂CH₂CH₂CH₂—**^{L1}, *^{L1}—C(O)NR^{L1a}—CH₂CH₂CH₂CH₂CH₂CH₂CH₂—**^{L1}, *^{L1}—C(O)NR^{L1a}CH₂CH₂CH₂CH₂CH₂CH₂CH₂CH₂—**^{L1}, *^{L1}—CH₂—NR^{L1a}C(O)—**^{L1}, *^{L1}—CH₂CH₂—NR^{L1a}C(O)—**^{L1}, *^{L1}—CH₂CH₂CH₂—NR^{L1a} C(O)—**^{L1}, *^{L1}—CH₂CH₂CH₂CH₂—NR^{L1a}C(O)—**^{L1}, *^{L1}—CH₂CH₂CH₂CH₂CH₂—NR^{L1a}C(O)—**^{L1}, *^{L1}—CH₂CH₂CH₂CH₂CH₂CH₂—NR^{L1a}C(O)—**^{L1}, *^{L1}—CH₂CH₂CH₂CH₂CH₂CH₂CH₂—NR^{L1a}C(O)—**^{L1}, *^{L1}—CH₂CH₂CH₂CH₂CH₂CH₂CH₂CH₂—NR^{L1a}C(O)—**^{L1}, *^{L1}—NR^{L1a}C(O)—CH₂—**^{L1}, *^{L1}—NR^{L1a}C(O)—CH₂CH₂—**^{L1}, *^{L1}—NR^{L1a}C(O)—CH₂CH₂CH₂—**^{L1}, *^{L1}—NR^{L1a}C(O)—CH₂CH₂CH₂CH₂—**^{L1}, *^{L1}—NR^{L1a}C(O)—CH₂CH₂CH₂CH₂CH₂—**^{L1}, *^{L1}—NR^{L1a}C(O)—CH₂CH₂CH₂CH₂CH₂CH₂—**^{L1}, *^{L1}—NR^{L1a}C(O)—CH₂CH₂CH₂CH₂CH₂CH₂CH₂—**^{L1}, *^{L1}—NR^{L1a}C(O)—CH₂CH₂CH₂CH₂CH₂CH₂CH₂CH₂—**^{L1}, *^{L1}—C(O)O—CH₂—**^{L1}, *^{L1}—C(O)O—CH₂CH₂—**^{L1}, *^{L1}—C(O)O—CH₂CH₂CH₂—**^{L1}, *^{L1}—C(O)O—CH₂CH₂CH₂CH₂—**^{L1}, *^{L1}—C(O)O—CH₂CH₂CH₂CH₂CH₂—**^{L1}, *^{L1}—C(O)O—CH₂CH₂CH₂CH₂CH₂CH₂—**^{L1}, *^{L1}—C(O)O—CH₂CH₂CH₂CH₂CH₂CH₂CH₂—**^{L1}, *^{L1}—C(O)O—CH₂CH₂CH₂CH₂CH₂CH₂CH₂CH₂—**^{L1}, *^{L1}—CH₂—C(O)O—**^{L1}, *^{L1}—CH₂CH₂—C(O)O—**^{L1}, *^{L1}—CH₂CH₂CH₂—C(O)O—**^{L1}, *^{L1}—CH₂CH₂CH₂CH₂—C(O)O—**^{L1}, *^{L1}—CH₂CH₂CH₂CH₂CH₂—C(O)O—**^{L1}, *^{L1}—CH₂CH₂CH₂CH₂CH₂CH₂—C(O)O—**^{L1}, *^{L1}—CH₂CH₂CH₂CH₂CH₂CH₂CH₂—C(O)O**^{L1}, *^{L1}—CH₂CH₂CH₂CH₂CH₂CH₂CH₂CH₂—C(O)O—**^{L1}, *^{L1}—OC(O)—CH₂—**^{L1}, *^{L1}—OC(O)—CH₂CH₂—**^{L1}, *^{L1}—OC(O)—CH₂CH₂CH₂—**^{L1}, *^{L1}—OC(O)—CH₂CH₂CH₂CH₂—**^{L1}, *^{L1}—OC(O)—CH₂CH₂CH₂CH₂CH₂—**^{L1}, *^{L1}—OC(O)—CH₂CH₂CH₂CH₂CH₂CH₂—**^{L1}, *^{L1}—OC(O)—CH₂CH₂CH₂CH₂CH₂CH₂CH₂—**^{L1}, *^{L1}—OC(O)—CH₂CH₂CH₂CH₂CH₂CH₂CH₂CH₂—**^{L1}, *^{L1}—CH₂—OC(O)—**^{L1}, *^{L1}—CH₂CH₂—OC(O)—**^{L1}, *^{L1}—CH₂CH₂CH₂—OC(O)—**^{L1}, *^{L1}—CH₂CH₂CH₂CH₂—OC(O)—**^{L1}, *^{L1}—CH₂CH₂CH₂CH₂CH₂—OC(O)—**^{L1}, *^{L1}—CH₂CH₂CH₂CH₂CH₂CH₂—OC(O)—**^{L1}, *^{L1}—CH₂CH₂CH₂CH₂CH₂CH₂CH₂—OC(O)—^{L1}, *^{L1}—CH₂CH₂CH₂CH₂CH₂CH₂CH₂CH₂—OC(O)—**^{L1}, 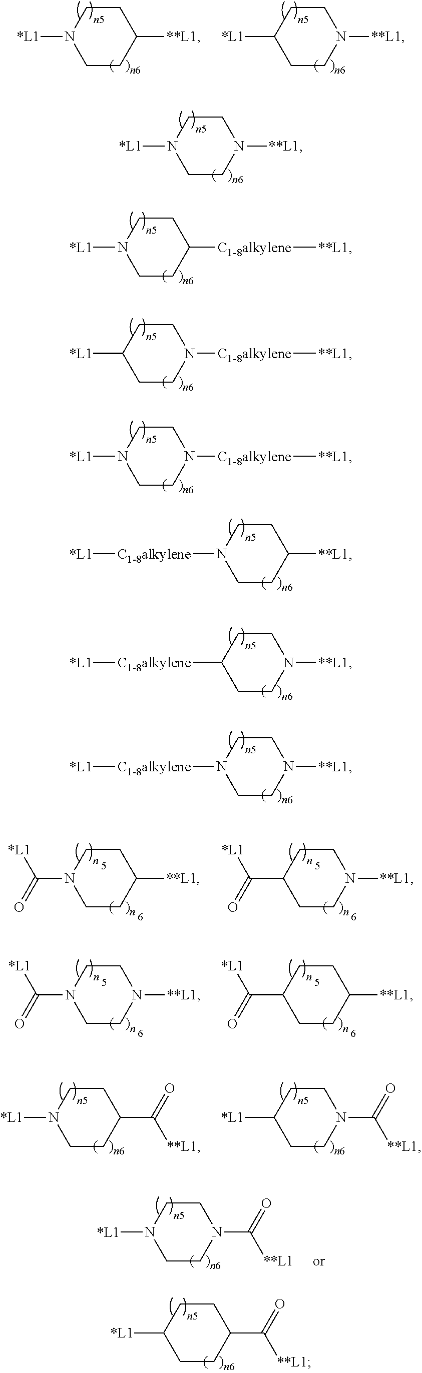
each of said —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH₂CH₂CH₂CH₂CH₂CH₂CH₂—, —CH₂CH₂CH₂CH₂CH₂CH₂CH₂CH₂—, —C₃-C₈cycloalkylene-, *^{L1}—O—CH₂—**^{L1}, *^{L1}—O—CH₂CH₂—**^{L1}, *^{L1}—O—CH₂CH₂CH₂—**^{L1}, *^{L1}—O—CH₂CH₂CH₂CH₂—**^{L1}, *^{L1}—O—CH₂CH₂CH₂CH₂CH₂—**^{L1}, *^{L1}—O—CH₂CH₂CH₂CH₂CH₂CH₂—**^{L1}, *^{L1}—O—CH₂CH₂CH₂CH₂CH₂CH₂CH₂—**^{L1}, *^{L1}—O—CH₂CH₂CH₂CH₂CH₂CH₂CH₂CH₂—**^{L1}, *^{L1}—CH₂—**^{L1}, *^{L1}—CH₂CH₂—O—**^{L1}, *^{L1}—CH₂CH₂CH₂—O—**^{L1}, *^{L1}—CH₂CH₂CH₂CH₂—O—**^{L1}, *^{L1}—CH₂CH₂CH₂CH₂CH₂—O—**^{L1}, *^{L1}—CH₂CH₂CH₂CH₂CH₂CH₂—O—**^{L1}, *^{L1}—CH₂CH₂CH₂CH₂CH₂CH₂CH₂—O—**^{L1}, *^{L1}—CH₂CH₂CH₂CH₂CH₂CH₂CH₂CH₂—O—**^{L1}, *^{L1}—S—CH₂—**^{L1}, *^{L1}—S—CH₂CH₂—**^{L1}, *^{L1}—S—CH₂CH₂CH₂—**^{L1}, *^{L1}—S—CH₂CH₂CH₂CH₂—**^{L1}, *^{L1}—S—CH₂CH₂CH₂CH₂CH₂—**^{L1}, *^{L1}—S—CH₂CH₂CH₂CH₂CH₂CH₂—**^{L1}, *^{L1}—S—CH₂CH₂CH₂CH₂CH₂CH₂CH₂—**^{L1}, *^{L1}—S—CH₂CH₂CH₂CH₂CH₂CH₂CH₂CH₂—**^{L1}, *^{L1}—CH₂—S—**^{L1}, *^{L1}—CH₂CH₂—S—**^{L1}, *^{L1}—CH₂CH₂CH₂—S—**^{L1}, *^{L1}—CH₂CH₂CH₂CH₂—S—**^{L1}, *^{L1}—CH₂CH₂CH₂CH₂CH₂—S—**^{L1}, *^{L1}—CH₂CH₂CH₂CH₂CH₂CH₂—S—**^{L1}, *^{L1}—CH₂CH₂CH₂CH₂CH₂CH₂CH₂—S—**^{L1}, *^{L1}—CH₂CH₂CH₂CH₂CH₂CH₂CH₂CH₂—S—**^{L1}, *^{L1}—O—C₃-C₈cycloalkylene-**^{L1}, *^{L1}—C₃-C₈cycloalkylene-O—**^{L1}, *^{L1}—S—C₃-C₈cycloalkylene-**^{L1}, *^{L1}—C₃-C₈cycloalkylene-S—**^{L1}, *^{L1}—O—CH₂—NR^{L1a}—**^{L1}, *^{L1}—O—CH₂CH₂—NR^{L1a}—**^{L1}, *^{L1}—O—CH₂CH₂CH₂—NR^{L1a}—**^{L1}, *^{L1}—O—CH₂CH₂CH₂CH₂—NR^{L1a}—**^{L1}, *^{L1}—O—CH₂CH₂CH₂CH₂CH₂—NR^{L1a}—**^{L1}, *^{L1}—O—CH₂CH₂CH₂CH₂CH₂CH₂—NR^{L1a}—**^{L1}, *^{L1}—O—CH₂CH₂CH₂CH₂CH₂CH₂CH₂—NR^{L1a}—**^{L1},*^{L1}—O—CH₂CH₂CH₂CH₂CH₂CH₂CH₂CH₂—NR^{L1a}—**^{L1}, *^{L1}—NR^{L1a}—CH₂—O—**^{L1}, *^{L1}—NR^{L1a}—CH₂CH₂—O—**^{L1}, *^{L1}—NR^{L1a}—CH₂CH₂CH₂—O—**^{L1}, *^{L1}—NR^{L1a}—CH₂CH₂CH₂CH₂—O—**^{L1}, *^{L1}—NR^{L1a}—CH₂CH₂CH₂CH₂CH₂—O—**^{L1}, *^{L1}—NR^{L1a}—CH₂CH₂CH₂CH₂CH₂CH₂—O—**^{L1}, *^{L1}—NR^{L1a}—CH₂CH₂CH₂CH₂CH₂CH₂CH₂—O—**^{L1}, *^{L1}—NR^{L1a}—CH₂CH₂CH₂CH₂CH₂CH₂CH₂CH₂—O—**^{L1}, *^{L1}—S—CH₂—NR^{L1a}—**^{L1}, *^{L1}—S—CH₂CH₂—NR^{L1a}—**^{L1}, *^{L1}—S—CH₂CH₂CH₂—NR^{L1a}—**^{L1}, *^{L1}—S—CH₂CH₂CH₂CH₂—NR^{L1a}—**^{L1}, *^{L1}—S—CH₂CH₂CH₂CH₂CH₂—NR^{L1a}—**^{L1}, *^{L1}—S—CH₂CH₂CH₂CH₂CH₂CH₂—NR^{L1a}—**^{L1}, *^{L1}—S—CH₂CH₂CH₂CH₂CH₂CH₂CH₂—NR^{L1a}—**^{L1}, *^{L1}—S—CH₂CH₂CH₂CH₂CH₂CH₂CH₂CH₂—NR^{L1a}**^{L1}, *^{L1}—NR^{L1a}—CH₂—S—**^{L1}, *^{L1}—NR^{L1a}—CH₂CH₂—S—**^{L1}, *^{L1}—NR^{L1a}—CH₂CH₂CH₂—S—**^{L1}, *^{L1}—NR^{L1a}—CH₂CH₂CH₂CH₂—S—**^{L1}, *^{L1}—NR^{L1a}—CH₂CH₂CH₂CH₂CH₂—S—**^{L1}, *^{L1}—NR^{L1a}—CH₂CH₂CH₂CH₂CH₂CH₂—S—**^{L1}, *^{L1}—NR^{L1a}—CH₂CH₂CH₂CH₂CH₂CH₂CH₂—S—**^{L1}, *^{L1}—NR^{L1a}—CH₂CH₂CH₂CH₂CH₂CH₂CH₂CH₂—S—**^{L1}, *^{L1}—O—CH₂—CO—**^{L1}, *^{L1}—O—CH₂CH₂—CO—**^{L1}, *^{L1}—O—CH₂CH₂CH₂—CO—**^{L1}, *^{L1}—O—CH₂CH₂CH₂CH₂—CO—**^{L1}, *^{L1}—O—CH₂CH₂CH₂CH₂CH₂—CO—**^{L1}, *^{L1}—O—CH₂CH₂CH₂CH₂CH₂CH₂—CO—**^{L1}, *^{L1}—O—CH₂CH₂CH₂CH₂CH₂CH₂CH₂—CO—**^{L1}, *^{L1}—O—CH₂CH₂CH₂CH₂CH₂CH₂CH₂CH₂—CO—**^{L1}, *^{L1}—CO—CH₂—O—**^{L1}, *^{L1}—CO—CH₂CH₂—O—**^{L1}, *^{L1}—CO—CH₂CH₂CH₂—O—**^{L1}, *^{L1}—CO—CH₂CH₂CH₂CH₂—O—**^{L1}, *^{L1}—CO—CH₂CH₂CH₂CH₂CH₂—O—**^{L1}, *^{L1}—CO—CH₂CH₂CH₂CH₂CH₂CH₂—O—**^{L1}, *^{L1}—CO—CH₂CH₂CH₂CH₂CH₂CH₂CH₂—O—**^{L1}, *^{L1}—CO—CH₂CH₂CH₂CH₂CH₂CH₂CH₂CH₂—O—**^{L1}, *^{L1}—S—CH₂—CO—**^{L1}, *^{L1}—S—CH₂CH₂—CO—**^{L1}, *^{L1}—S—CH₂CH₂CH₂—CO—**^{L1}, *^{L1}—S—CH₂CH₂CH₂CH₂—CO—**^{L1}, *^{L1}—S—CH₂CH₂CH₂CH₂CH₂—CO—**^{L1}, *^{L1}—S—CH₂CH₂CH₂CH₂CH₂CH₂—CO—**^{L1}, *^{L1}—S—CH₂CH₂CH₂CH₂CH₂CH₂CH₂—CO—**^{L1}, *^{L1}—S—CH₂CH₂CH₂CH₂CH₂CH₂CH₂CH₂—CO—**^{L1}, *^{L1}—CO—CH₂—S—**^{L1}, *^{L1}—CO—CH₂CH₂—S—**^{L1}, *^{L1}—CO—CH₂CH₂CH₂—S—**^{L1}, *^{L1}—CO—CH₂CH₂CH₂CH₂—S—**^{L1}, *^{L1}—CO—CH₂CH₂CH₂CH₂CH₂—S—**^{L1}, *^{L1}—CO—CH₂CH₂CH₂CH₂CH₂CH₂—S—**^{L1}, *^{L1}—CO—CH₂CH₂CH₂CH₂CH₂CH₂CH₂—S—**^{L1}, *^{L1}—CO—CH₂CH₂CH₂CH₂CH₂CH₂CH₂CH₂—S—**^{L1}, *^{L1}—C(O)—CH₂—**^{L1}, *^{L1}—C(O)—CH₂CH₂—**^{L1}, *^{L1}—C(O)—CH₂CH₂CH₂—**^{L1}, *^{L1}—C(O)—CH₂CH₂CH₂CH₂—**^{L1}, *^{L1}—C(O)—CH₂CH₂CH₂CH₂CH₂—**^{L1}, *^{L1}—C(O)—CH₂CH₂CH₂CH₂CH₂CH₂—**^{L1}, *^{L1}—C(O)—CH₂CH₂CH₂CH₂CH₂CH₂CH₂—**^{L1}, *^{L1}—C(O)—CH₂CH₂CH₂CH₂CH₂CH₂CH₂CH₂—**^{L1}, *^{L1}—CH₂—C(O)—**^{L1}, *^{L1}—CH₂CH₂—C(O)—**^{L1}, *^{L1}—CH₂CH₂CH₂—C(O)—**^{L1}, *^{L1}—CH₂CH₂CH₂CH₂—C(O)—**^{L1}, *^{L1}—CH₂CH₂CH₂CH₂CH₂—C(O)—**^{L1}, *^{L1}—CH₂CH₂CH₂CH₂CH₂CH₂—C(O)—**^{L1}, *^{L1}—CH₂CH₂CH₂CH₂CH₂CH₂CH₂—C(O)—**^{L1}, *^{L1}—CH₂CH₂CH₂CH₂CH₂CH₂CH₂CH₂—C(O)—**^{L1}, *^{L1}—CH₂—NR^{L1a}—**^{L1}, *^{L1}—CH₂CH₂—NR^{L1a}—**^{L1}, *^{L1}—CH₂CH₂CH₂—NR^{L1a}—**^{L1}, *^{L1}—CH₂CH₂CH₂CH₂—NR^{L1a}—**^{L1}, *^{L1}—CH₂CH₂CH₂CH₂CH₂—NR^{L1a}—**^{L1}, *^{L1}—CH₂CH₂CH₂CH₂CH₂CH₂—NR^{L1a}—**^{L1},*^{L1}—CH₂CH₂CH₂CH₂CH₂CH₂CH₂—NR^{L1a}—**^{L1}, *^{L1}—CH₂CH₂CH₂CH₂CH₂CH₂CH₂CH₂—NR^{L1a}—**^{L1}, *^{L1}—NR^{L1a}—CH₂—**^{L1}, *^{L1}—NR^{L1a}CH₂CH₂—**^{L1}, *^{L1}—NR^{L1a}—CH₂CH₂CH₂—**^{L1}, *^{L1}—NR^{L1a}—CH₂CH₂CH₂CH₂—**^{L1}, *^{L1}—NR^{L1a}—CH₂CH₂CH₂CH₂CH₂—**^{L1}, *^{L1}—NR^{L1a}—CH₂CH₂CH₂CH₂CH₂CH₂—**^{L1}, *^{L1}—NR^{L1a}—CH₂CH₂CH₂CH₂CH₂CH₂CH₂—**^{L1}, *^{L1}—NR^{L1a}—CH₂CH₂CH₂CH₂CH₂CH₂CH₂CH₂—**^{L1}, *^{L1}—CH₂—C(O)NR^{L1a}—**^{L1}, *^{L1}—CH₂CH₂—C(O)NR^{L1a}—**^{L1}, *^{L1}—CH₂CH₂CH₂—C(O)NR^{L1a}—**^{L1}, *^{L1}—CH₂CH₂CH₂CH₂—C(O)NR^{L1a}—**^{L1}, *^{L1}—CH₂CH₂CH₂CH₂CH₂—C(O)NR^{L1a}—**^{L1}, *^{L1}—CH₂CH₂CH₂CH₂CH₂CH₂—C(O)NR^{L1a}—**^{L1}, *^{L1}—CH₂CH₂CH₂CH₂CH₂CH₂CH₂—C(O)NR^{L1a}—**^{L1}, *^{L1}—CH₂CH₂CH₂CH₂CH₂CH₂CH₂CH₂—C(O)NR^{L1a}—**^{L1}, *^{L1}—C(O)NR^{L1a}—CH₂—**^{L1}, *^{L1}—C(O)NR^{L1a}—CH₂CH₂—**^{L1}, *^{L1}—C(O)NR^{L1a}—CH₂CH₂CH₂—**^{L1}, *^{L1}—C(O)NR^{L1a}—CH₂CH₂CH₂CH₂—**^{L1}, *^{L1}—C(O)NR^{L1a}—CH₂CH₂CH₂CH₂CH₂—**^{L1}, *^{L1}—C(O)NR^{L1a}—CH₂CH₂CH₂CH₂CH₂CH₂—**^{L1}, *^{L1}—C(O)NR^{L1a}—CH₂CH₂CH₂CH₂CH₂CH₂CH₂—**^{L1}, *^{L1}—C(O)NR^{L1a}CH₂CH₂CH₂CH₂CH₂CH₂CH₂CH₂—**^{L1}, *^{L1}—CH₂—NR^{L1a}C(O)—**^{L1}, *^{L1}—CH₂CH₂—NR^{L1a}C(O)—**^{L1}, *^{L1}—CH₂CH₂CH₂—NR^{L1a} C(O)—**^{L1}, *^{L1}—CH₂CH₂CH₂CH₂—NR^{L1a}C(O)—**^{L1}, *^{L1}—CH₂CH₂CH₂CH₂CH₂—NR^{L1a}C(O)—**^{L1}, *^{L1}—CH₂CH₂CH₂CH₂CH₂CH₂—NR^{L1a}C(O)—**^{L1}, *^{L1}—CH₂CH₂CH₂CH₂CH₂CH₂CH₂—NR^{L1a}C(O)—**^{L1}, *^{L1}—CH₂CH₂CH₂CH₂CH₂CH₂CH₂CH₂—NR^{L1a}C(O)—**^{L1}, *^{L1}—NR^{L1a}C(O)—CH₂—**^{L1}, *^{L1}—NR^{L1a}C(O)—CH₂CH₂—**^{L1}, *^{L1}—NR^{L1a}C(O)—CH₂CH₂CH₂—**^{L1}, *^{L1}—NR^{L1a}C(O)—CH₂CH₂CH₂CH₂—**^{L1}, *^{L1}—NR^{L1a}C(O)—CH₂CH₂CH₂CH₂CH₂—**^{L1}, *^{L1}—NR^{L1a}C(O)—CH₂CH₂CH₂CH₂CH₂CH₂—**^{L1}, *^{L1}—NR^{L1a}C(O)—CH₂CH₂CH₂CH₂CH₂CH₂CH₂—**^{L1}, *^{L1}—NR^{L1a}C(O)—CH₂CH₂CH₂CH₂CH₂CH₂CH₂CH₂—**^{L1}, *^{L1}—C(O)O—CH₂—**^{L1}, *^{L1}—C(O)O—CH₂CH₂—**^{L1}, *^{L1}—C(O)O—CH₂CH₂CH₂—**^{L1}, *^{L1}—C(O)O—CH₂CH₂CH₂CH₂—**^{L1}, *^{L1}—C(O)O—CH₂CH₂CH₂CH₂CH₂—**^{L1}, *^{L1}—C(O)O—CH₂CH₂CH₂CH₂CH₂CH₂—**^{L1}, *^{L1}—C(O)O—CH₂CH₂CH₂CH₂CH₂CH₂CH₂—**^{L1}, *^{L1}—C(O)O—CH₂CH₂CH₂CH₂CH₂CH₂CH₂CH₂—**^{L1}, *^{L1}—CH₂—C(O)O—**^{L1}, *^{L1}—CH₂CH₂—C(O)O—**^{L1}, *^{L1}—CH₂CH₂CH₂—C(O)O—**^{L1}, *^{L1}—CH₂CH₂CH₂CH₂—C(O)O—**^{L1}, *^{L1}—CH₂CH₂CH₂CH₂CH₂—C(O)O—**^{L1}, *^{L1}—CH₂CH₂CH₂CH₂CH₂CH₂—C(O)O—**^{L1}, *^{L1}—CH₂CH₂CH₂CH₂CH₂CH₂CH₂—C(O)O—**^{L1}, *^{L1}—CH₂CH₂CH₂CH₂CH₂CH₂CH₂CH₂—C(O)O—**^{L1}, *^{L1}—OC(O)—CH₂—**^{L1}, *^{L1}—OC(O)—CH₂CH₂—**^{L1}, *^{L1}—OC(O)—CH₂CH₂CH₂—**^{L1}, *^{L1}—OC(O)—CH₂CH₂CH₂CH₂—**^{L1}, *^{L1}—OC(O)—CH₂CH₂CH₂CH₂CH₂—**^{L1}, *^{L1}—OC(O)—CH₂CH₂CH₂CH₂CH₂CH₂—**^{L1}, *^{L1}—OC(O)—CH₂CH₂CH₂CH₂CH₂CH₂CH₂—**^{L1}, *^{L1}—OC(O)—CH₂CH₂CH₂CH₂CH₂CH₂CH₂CH₂—**^{L1}, *^{L1}—CH₂—OC(O)—**^{L1}, *^{L1}—CH₂CH₂—OC(O)—**^{L1}, *^{L1}—CH₂CH₂CH₂—OC(O)—**^{L1}, *^{L1}—CH₂CH₂CH₂CH₂—OC(O)—**^{L1}, *^{L1}—CH₂CH₂CH₂CH₂CH₂—OC(O)—**^{L1}, *^{L1}—CH₂CH₂CH₂CH₂CH₂CH₂—OC(O)—**^{L1}, *^{L1}—CH₂CH₂CH₂CH₂CH₂CH₂CH₂—OC(O)—**^{L1}, *^{L1}—CH₂CH₂CH₂CH₂CH₂CH₂CH₂CH₂—OC(O)—**^{L1}, 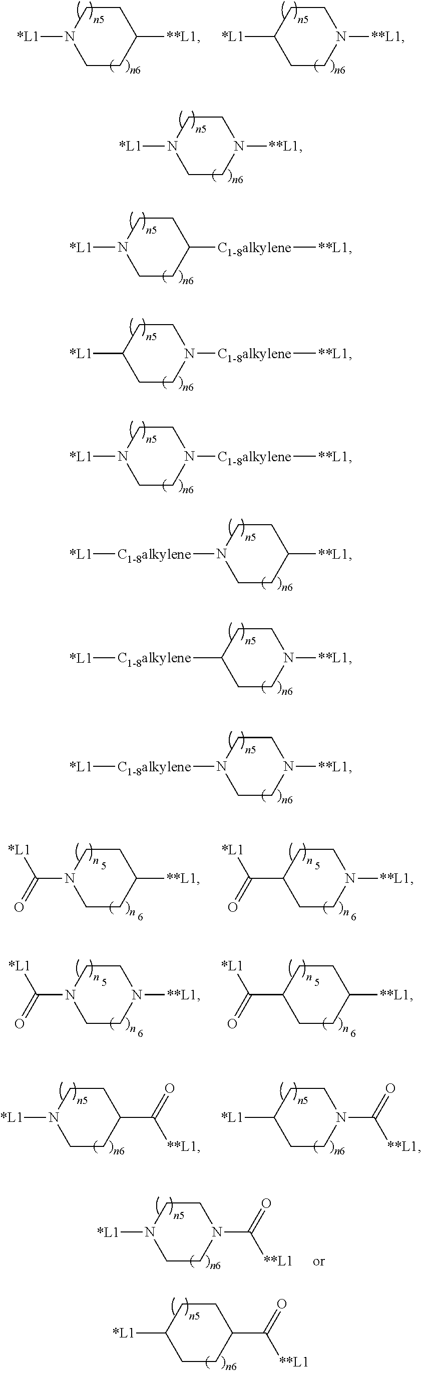
is optionally substituted with at least one R^{L1b};
n5 and n6 are each independently 0 or 1;
R^{L1a} is selected from hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl, each of said —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl is optionally substituted with at least one substituent R^{L1c};
each of said R^{L1b} and R^{L1c} is independently halogen, hydroxy, —C₁₋₈alkyl, —C₁₋₈alkoxy, —C₂₋₈alkenyl, —C₂₋₈alkynyl, C₃-C₈cycloalkyl, 3- to 8-membered heterocyclyl, C₆-C₁₂aryl or 5- to 12-membered heteroaryl; or
two R^{L1b} or two R^{L1c} together with the atoms to which they are attached, form a 3- to 6-membered unsaturated or saturated ring, said ring comprising 0-3 heteroatoms independently selected from nitrogen, oxygen or sulfur; said ring is optionally substituted with at least one substituent halogen, hydroxy, —C₁₋₈alkyl, —C₁₋₈alkoxy, —C₂₋₈alkenyl, —C₂₋₈alkynyl, C₃-C₈cycloalkyl, 3- to 8-membered heterocyclyl, C₆-C₁₂aryl or 5- to 12-membered heteroaryl.

17. The compound of claim 1, wherein L₁ is selected from a single bod,

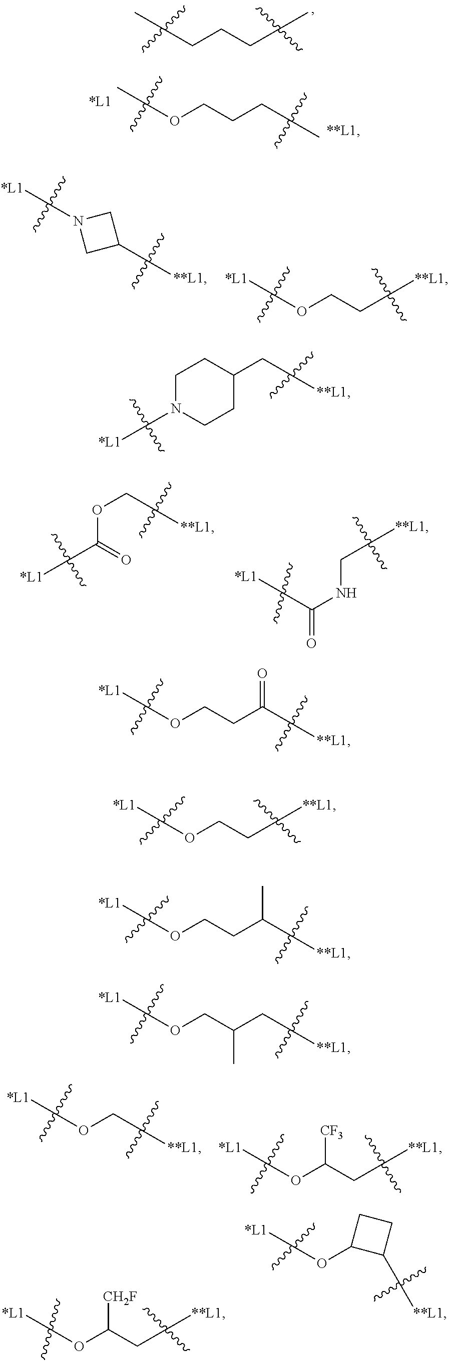

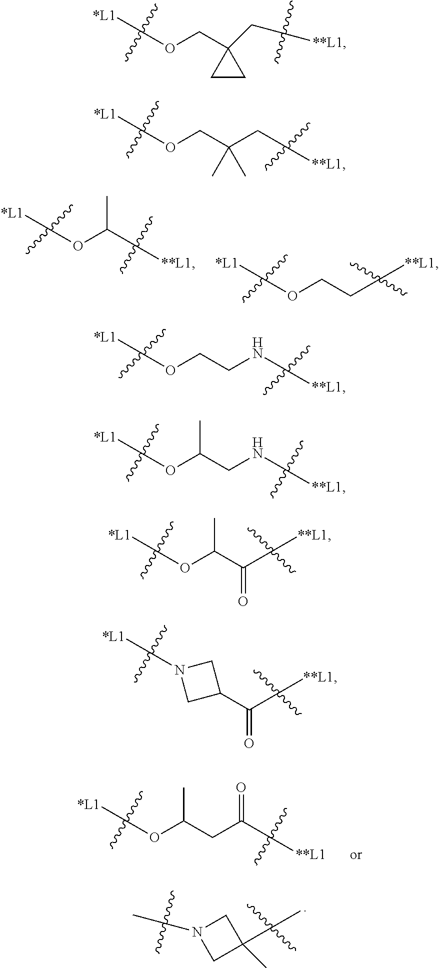

18. The compound of claim 1, wherein R₈ is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 7- to 9-membered spiro-heterocylic ring comprising one or two or three nitrogen atoms as the ring members, oxazolidinyl, imidazolidinyl, thiazolidinyl, pyrazolidinyl, morpholinyl, thiomorpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, phenyl, tetrahydropyridinyl, azetidinyl, pyrrolidinyl, octahydroindolizinyl, octahydroquinolizinyl, hexahydro-1H-pyrrolizinyl, tetrahydroisoquinolinyl, tetrahydropyridyl, oxo, —CN, —OR₈ₐ or —NR₈ₐR_{8b};
wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, 7- to 9-membered spiro-heterocylic ring comprising one or two or three nitrogen atoms as the ring members, oxazolidinyl, imidazolidinyl, thiazolidinyl, pyrazolidinyl, morpholinyl, thiomorpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, phenyl or tetrahydropyridinyl is optionally substituted with at least one substituent R_{8c};
R₈ₐ and R_{8b} are each independently selected from hydrogen, methyl, ethyl, propyl, butyl, pentyl, hexyl, heptyl, —C₂₋₈alkenyl, —C₂₋₈alkynyl, C₁₋₈alkoxy-C₁₋₈alkyl-, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl or oxo; or
R₈ₐ and R_{8b} together with the carbon atoms to which they are attached, form a 3- to 8-membered unsaturated or saturated ring, said ring comprising 0, 1, 2, or 3 heteroatoms independently selected from nitrogen, oxygen or sulfur; said ring is optionally substituted with at least one substituent R₈ₐ;
R_{8c}, at each occurrence, is independently halogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, —C₂₋₈alkenyl, —C₂₋₈alkynyl, oxo, —CN, —OR₈ₑ or —NR₈ₑR_{8f}; or
two R_{8c} together with the carbon atoms to which they are attached, form a 3- to 8-membered unsaturated or saturated ring, said ring comprising 0, 1, 2, or 3 heteroatoms independently selected from nitrogen, oxygen or sulfur; said ring is optionally substituted with at least one substituent R_{8d}; or two R_{8c} together with the atoms to which they are attached form a bridge containing one, two, three, or four —CH2- moieties in addition to the two bridgeheads, wherein one of the —CH₂— moiety is optionally replaced with —O—, —S— or —NH—;
R_{8d} is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, oxo, —CN, —OR_{8g}, —COR_{8g}, —CO₂R_{8g}, —CONR_{8g}R₈ₕ, —NR_{8g}R₈ₕ, —NR_{8g}COR₈ₕ or —NR_{8g}CO₂R₈ₕ, wherein each of methyl, ethyl, propyl, butyl, pentyl, hexyl, heptyl, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl or pyrazinyl is optionally substituted with at least one substituent R₈ᵢ;
R₈ₑ, R_{8f}, R_{8g}, R₈ₕ and R₈ᵢ are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, oxazolidinyl, imidazolidinyl, thiazolidinyl, pyrazolidinyl, morpholinyl, piperidinyl, piperazinyl, oxazinyl, imidazolyl, thiazolyl, oxazolyl, pyridyl, pyrimidinyl, pyrazinyl, pyridazinyl, phenyl, pyrrolyl, imidazolyl, pyrazolyl, thiazolyl, oxazolyl, triazolyl, thiophenyl, furanyl, pyridyl, pyrimidinyl, pyrazinyl, —C₂₋₈alkenyl, —C₂₋₈alkynyl, C₁₋₈alkoxy-C₁₋₈alkyl- or C₃-C₈cycloalkyl.

19. The compound of claim 1, wherein R₈ is

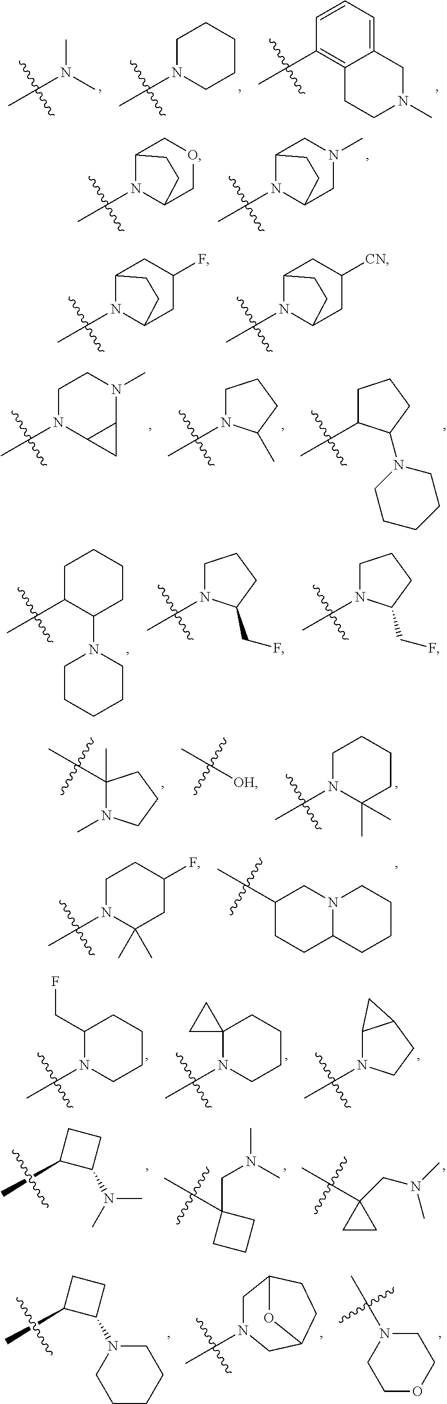

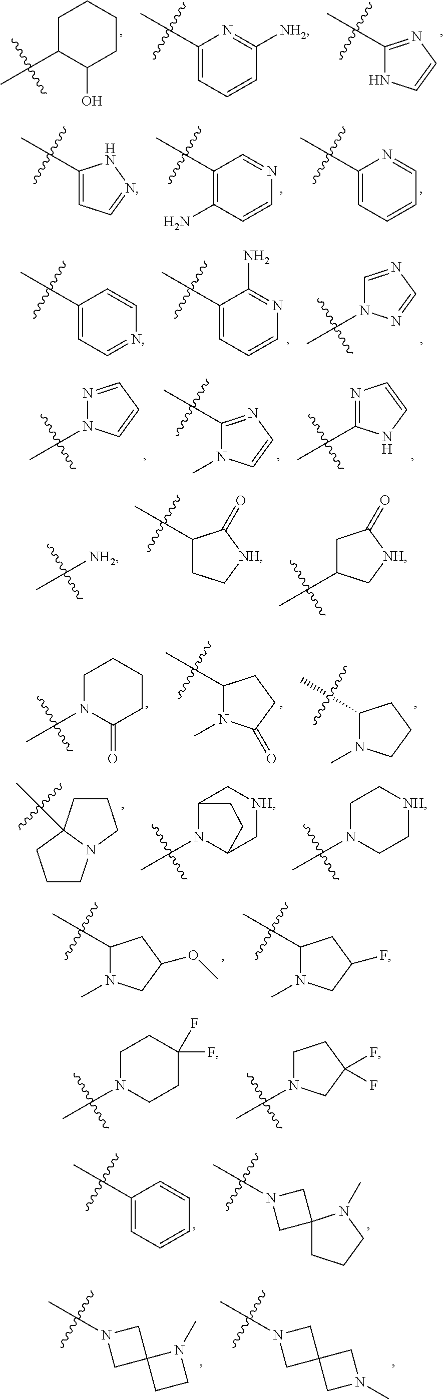

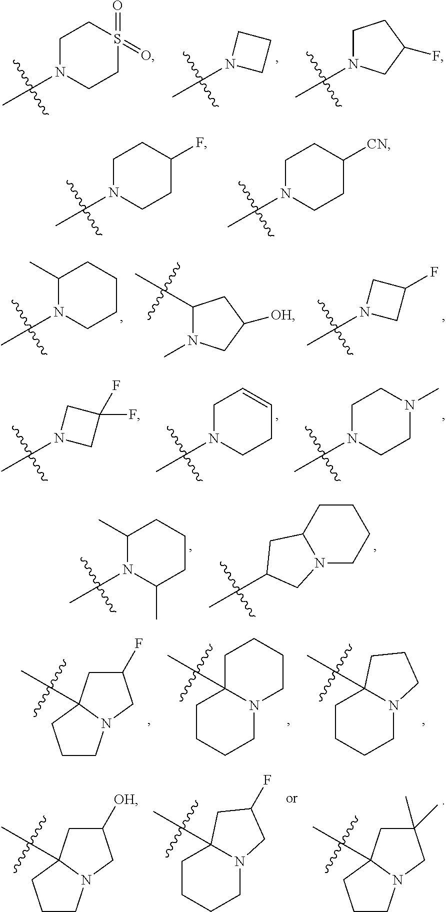

20. The compound of claim 1, wherein the compound is selected from Table 1.

21. A bridged compound having the following formula (VIII): 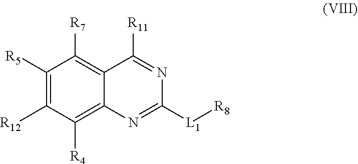
or a pharmaceutically acceptable salt, tautomer, isotopologue, stereoisomer or prodrug thereof, wherein
R₄ is methyl, or halogen;
R₅ is H, halogen, substituted or unsubstituted C₁₋₈alkyl, substituted or unsubstituted C₃₋₁₀cycloalkyl, substituted or unsubstituted C₂₋₁₀alkenyl, halogen, —CF₃, —O—CF₃ or —S—CF₃;
R₇ is hydrogen;
R₈ is substituted or unsubstituted C₃₋₁₀cycloalkyl, substituted or unsubstituted C₆₋₁₀aryl substituted or unsubstituted heterocyclyl, substituted or unsubstituted heteroaryl, —NH₂, substituted or unsubstituted —NHC₁₋₈alkyl, substituted or unsubstituted —N(C₁₋₈alkyl)₂, aminocarbonyl, hydroxyl, substituted or unsubstituted —NH-heteroaryl, —CF₃ or hydroxyl;
L₁ is —O—, substituted or unsubstituted —OC₁₋₈alkyl, substituted or unsubstituted heterocyclyl, or substituted or unsubstituted heteroaryl;
R₁₁ is substituted or unsubstituted C₃₋₁₀cycloalkyl, substituted or unsubstituted C₆₋₁₀aryl substituted or unsubstituted heterocyclyl, or substituted or unsubstituted heteroaryl; and
R₁₂ is substituted or unsubstituted C₃₋₁₀cycloalkyl, substituted or unsubstituted C₆₋₁₀aryl substituted or unsubstituted heterocyclyl, or substituted or unsubstituted heteroaryl.

22. A bridged compound having the following formula (IX): 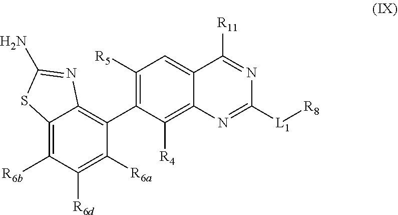
or a pharmaceutically acceptable salt, tautomer, isotopologue, stereoisomer or prodrug thereof,
wherein R₄ is F, Cl or methyl;
R₅ is —CF₃, —Cl, —OCF₃, —SCF₃, —CF₂CH₃, —CH₂CHF₂, —CN, —CHF₂, ethyl, isopropyl, 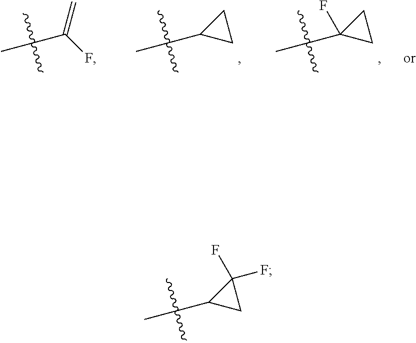
R₆ₐ is H, F, Cl or methyl;
R_{6b} is H, F, Cl or methyl;
R_{6d} is H, F, Cl or methyl;
-L₁-R₈ is 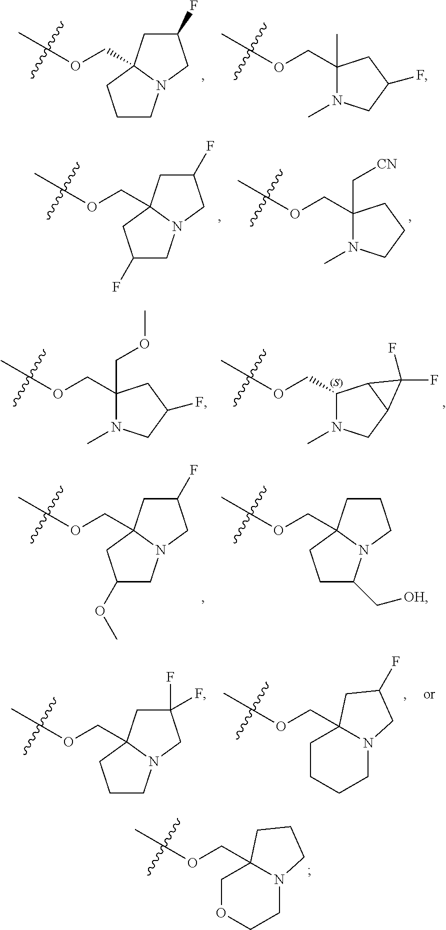
R₁₁ is 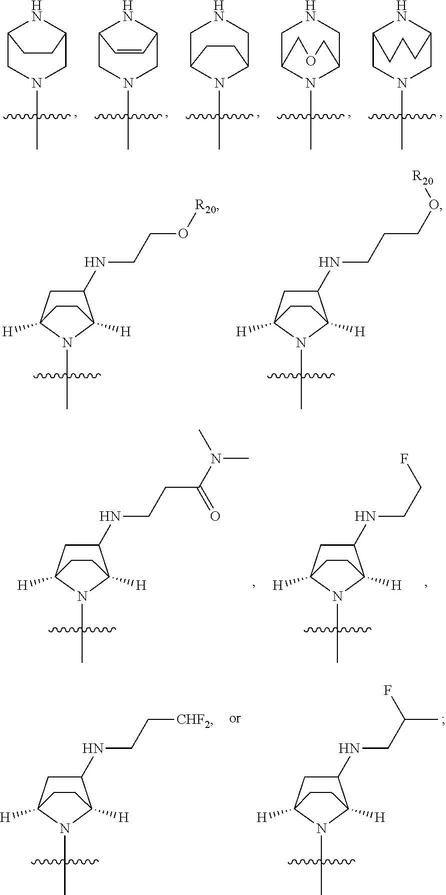
and
R₂₀ is methyl, —CHF₂, CF₃, —CH₂F, or —CD₃.

23. A bridged compound having the following formulas (Xa) or (Xb): 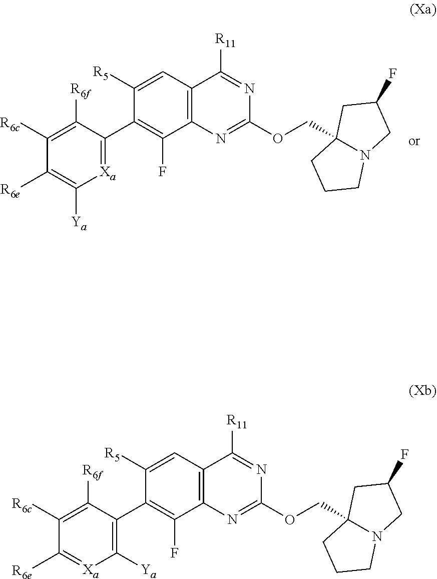
or a pharmaceutically acceptable salt, tautomer, isotopologue, stereoisomer or prodrug thereof,
wherein Xₐ is N, or CH;
Yₐ is H, F, Cl, —NH₂ or —OH;
R₅ is —CF₃, —F, —Cl, —OCF₃, —SCF₃, —CF₂CH₃, —CH₂CHF₂, —CN, —CHF₂, ethyl, isopropyl, 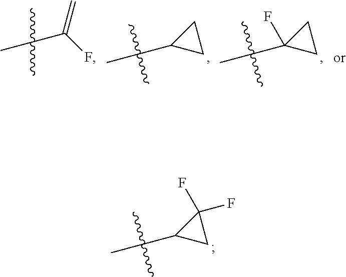
R_{6c} is H, F, Cl, methyl, ethyl, CHF₂, or CF₃;
R₆ₑ is H, F, or Cl;
R_{6f} is H, F, Cl, methyl, —CF₃, —CF₂CH₃, isopropyl, cyclopropyl, or methylcyclopropyl;
R₁₁ is 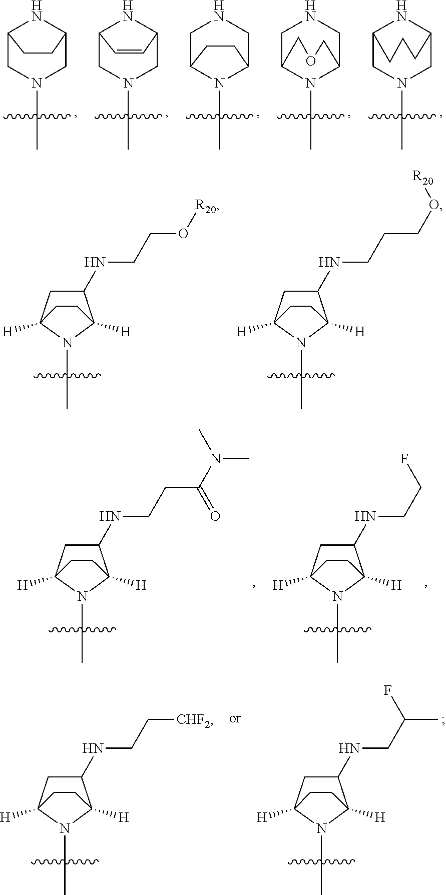
and
R₂₀ is methyl, —CHF₂, CF₃, —CH₂F, or —CD₃.

24. A bridged compound having the following formula (XI): 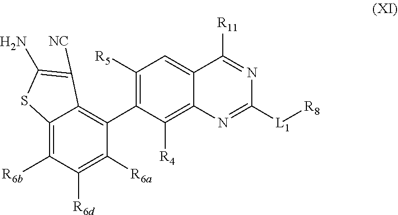
or a pharmaceutically acceptable salt, tautomer, isotopologue, stereoisomer or prodrug thereof,
wherein
R₄ is F, Cl or methyl;
R₅ is —CF₃, —Cl, —OCF₃, —SCF₃—CF₂CH₃, —CH₂CHF₂, —CN, —CHF₂, ethyl, isopropyl, 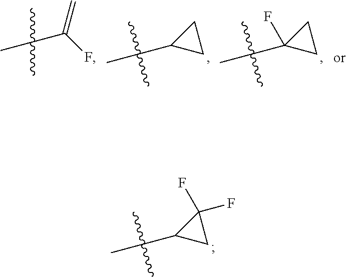
R₆ₐ is H, F, Cl or methyl;
R_{6b} is H, F, Cl or methyl;
R_{6d} is H, F, Cl or methyl;
-L₁-R₈ is 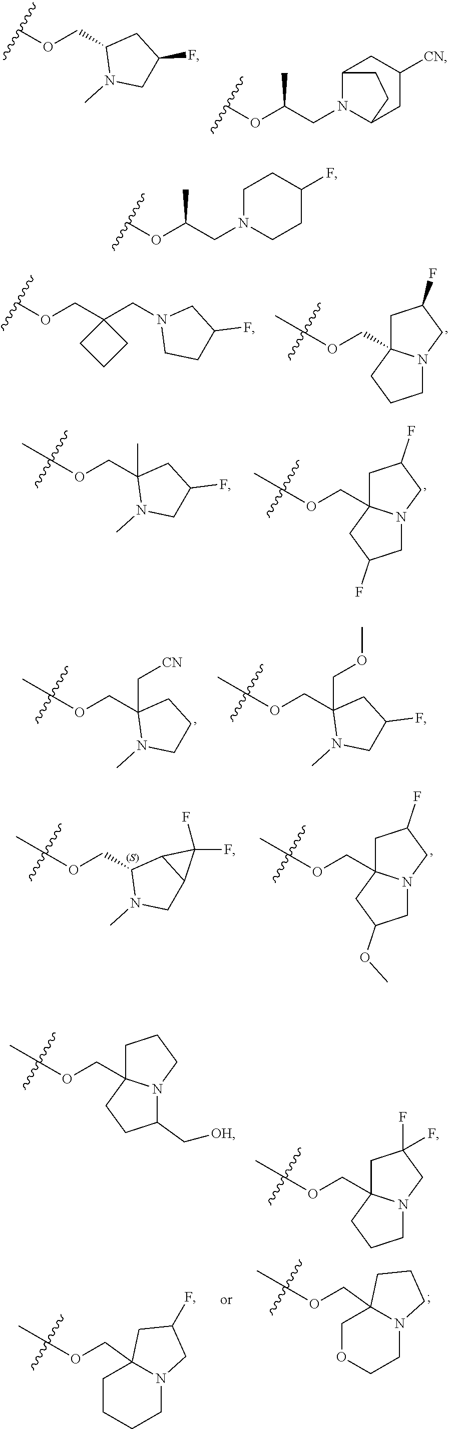
R₁₁ is 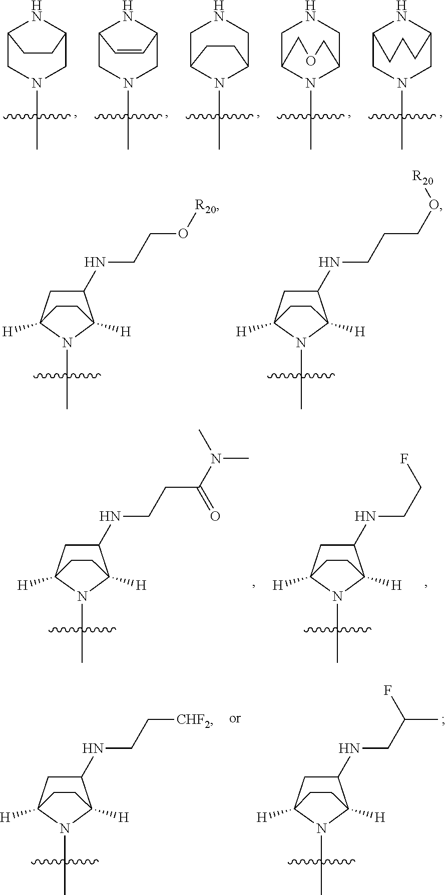
and
R₂₀ is methyl, —CHF₂, CF₃, —CH₂F, or —CD₃.

25. The bridged compound of claim 0,
wherein
R₄ is F, Cl or methyl;
R₅ is —CF₃, or —Cl;
R₆ₐ is H, F, Cl or methyl;
R_{6b} is H, F, Cl or methyl;
R_{6d} is H, F, Cl or methyl;
-L₁-R₈ is 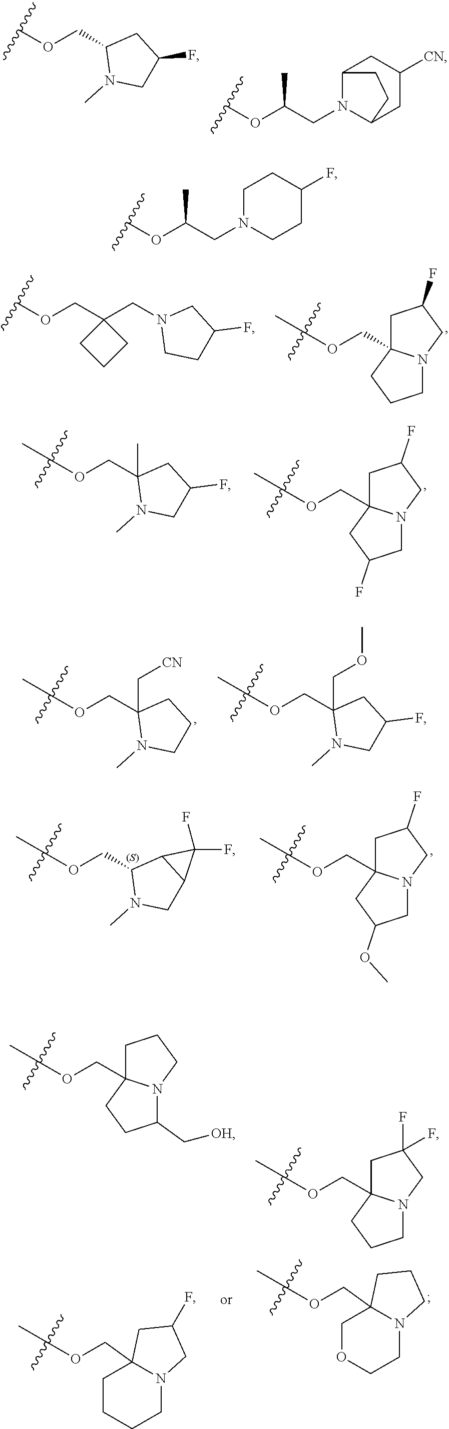
R₁₁ is 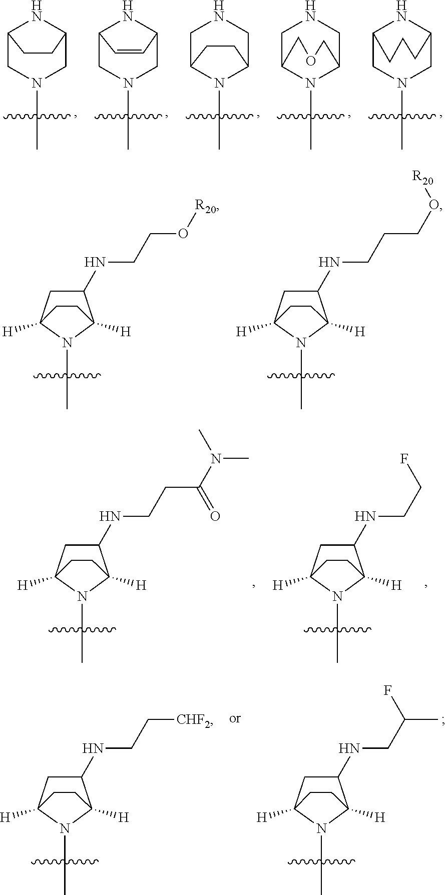
and
R₂₀ is methyl, —CHF₂, CF₃, —CH₂F, or —CD₃.

26. A bridged compound having formula (IX): 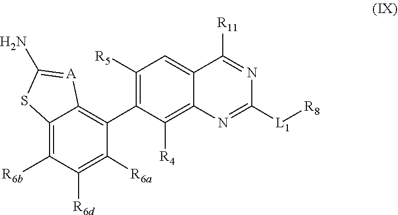
or a pharmaceutically acceptable salt, tautomer, or stereoisomer thereof,
wherein
wherein A is N or C—CN;
R₄ is H, F, Cl or methyl;
R₅ is —CF₃, —Cl, —OCF₃, —OCF₂Cl, —SCF₃, —H, —F, —Br, —I, —CF₂CH₃, —CH₂CHF₂, —CH₂CF₃, —CN, —CHF₂, ethyl, isopropyl, 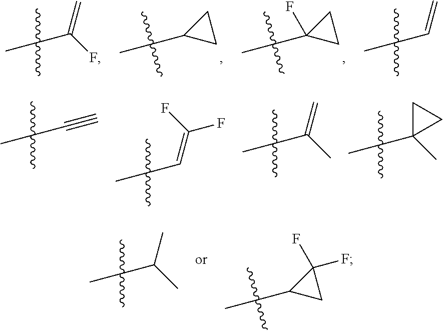
R₆ₐ is H, F, Cl or methyl;
R_{6b} is H, F, Cl or methyl;
R_{6d} is H, F, Cl or methyl;
-L₁- is —O—;
R₈ is unsubstituted or substituted alkyl, unsubstituted or substituted cycloalkyl, unsubstituted or substituted cycloalkylalkyl, unsubstituted or substituted aryl, unsubstituted or substituted aralkyl, unsubstituted or substituted heterocyclyl, unsubstituted or substituted heterocyclylalkyl, unsubstituted or substituted heteroaryl, unsubstituted or substituted heteroaralkyl, unsubstituted or substituted spirocyclic ring, or unsubstituted or substituted -alkyl-spirocyclic ring,
R₁₁ is 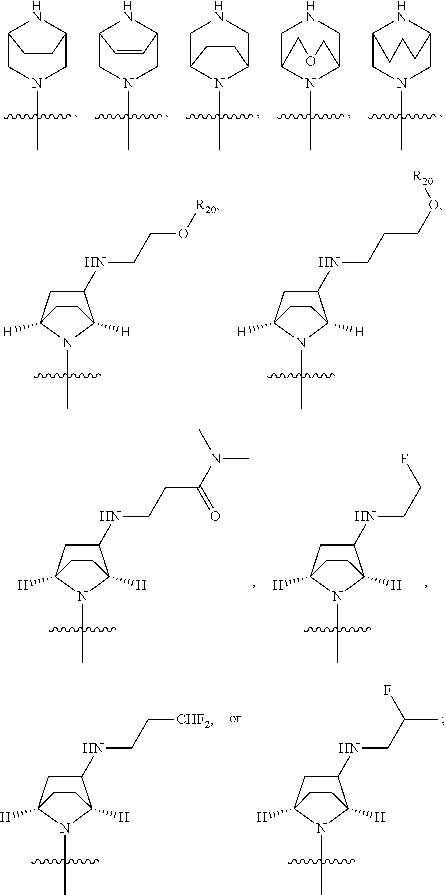
R₂₀ is methyl, —CHF₂, CF₃, —CH₂F, or —CD₃.

27. The bridged compound of claim 0,
wherein
R₄ is F, Cl;
R₅ is —CF₃;
R₆ₐ is H;
R_{6b} is F;
R_{6d} is H.

28. The bridged compound of claim 0,
wherein
R₄ is F;
R₅ is —CF₃;
R₆ₐ is H;
R_{6b} is F;
R_{6d} is H;
R₁₁ is

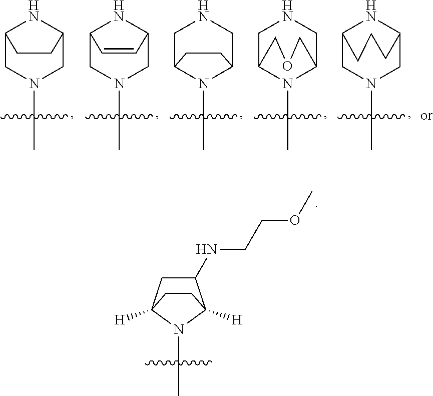

29. The bridged compound of claim 0,
wherein
R₄ is F, Cl or methyl;
R₅ is —CF₃, —Cl, —OCF₃, —OCF₂Cl, —SCF₃, —H, —F, —Br, —I, —CF₂CH₃, —CH₂CHF₂, —CH₂CF₃, —CN, —CHF₂, ethyl, isopropyl, 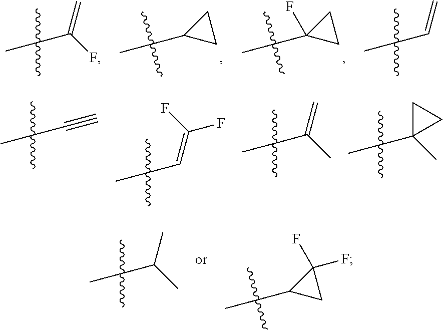
R₆ₐ is H, F, Cl or methyl;
R_{6b} is F, Cl or methyl;
R_{6d} is H;
-L₁-R₈ is 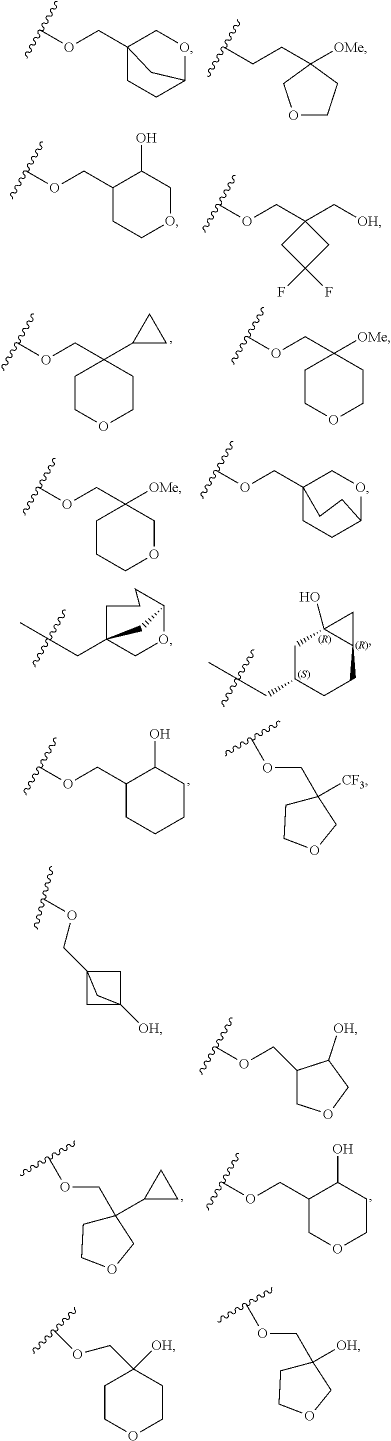 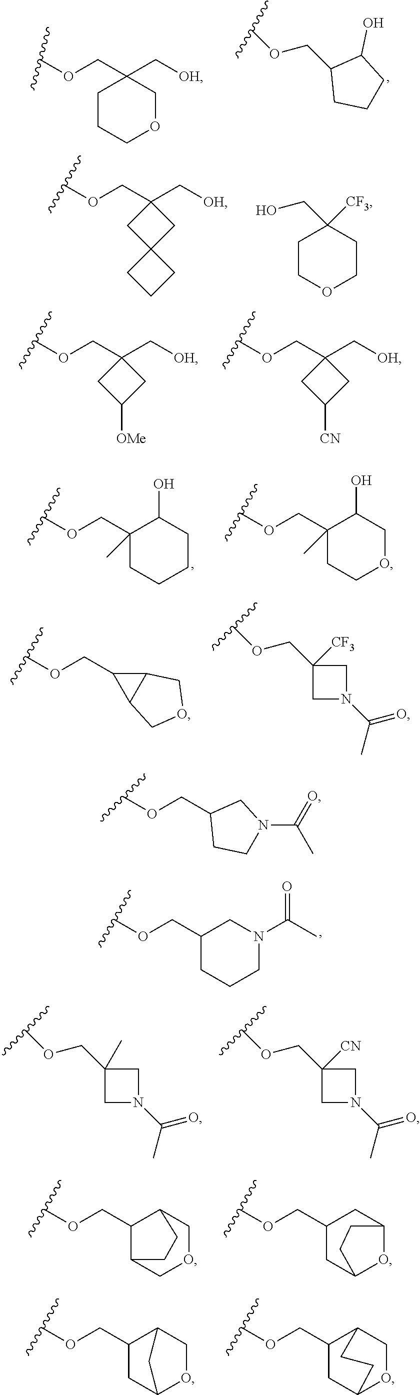 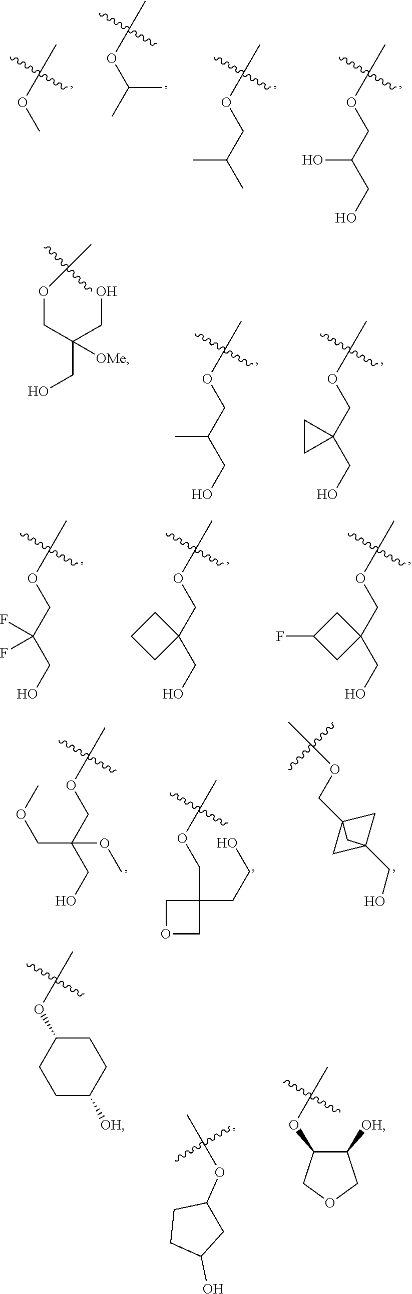 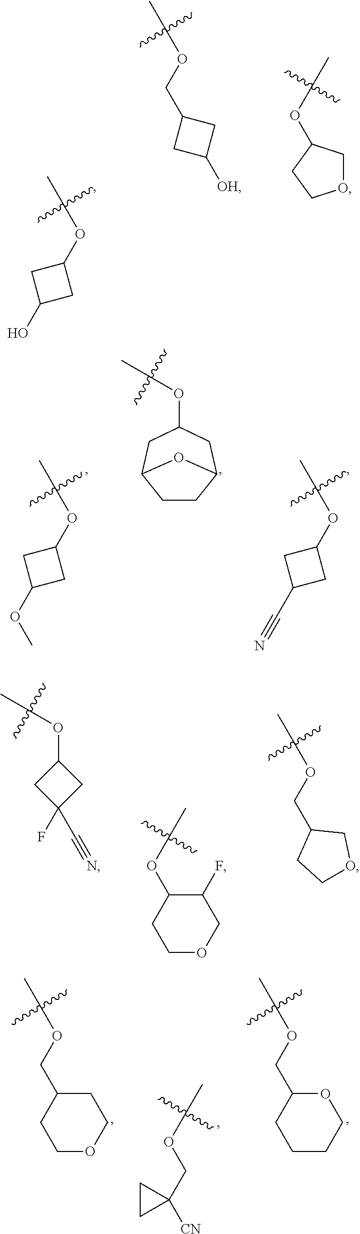 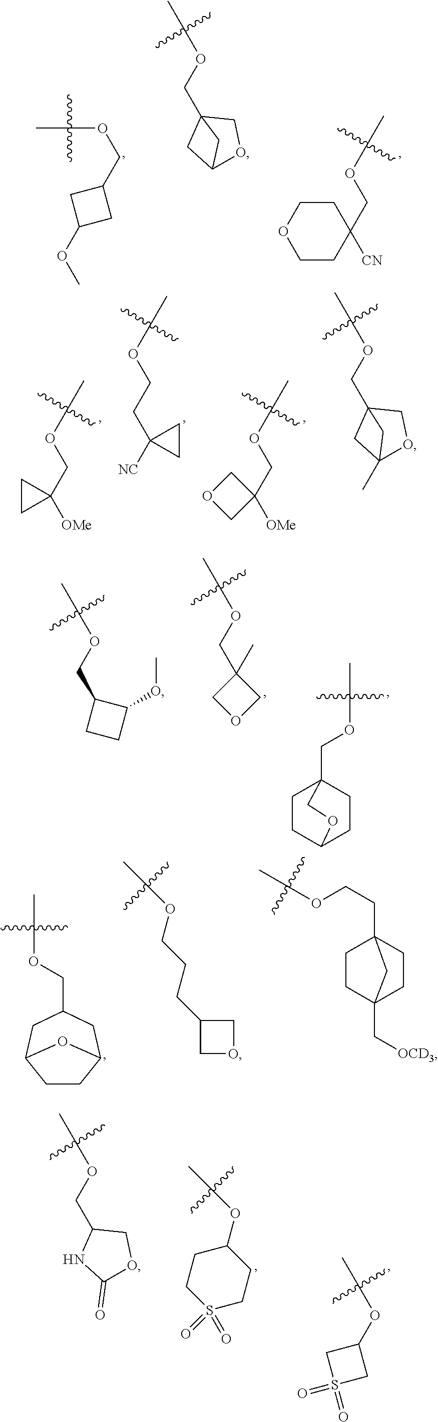 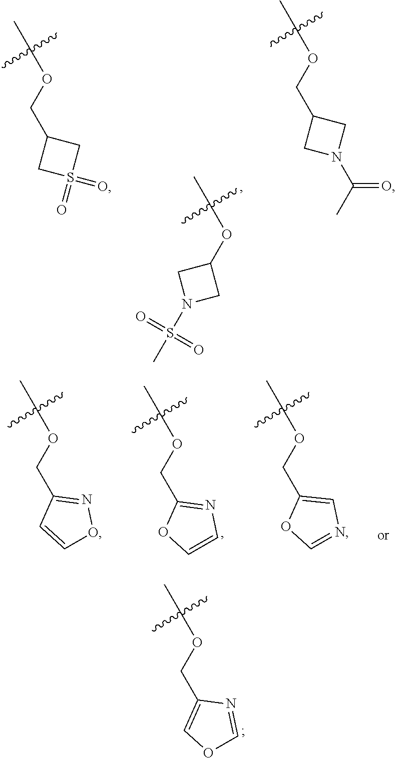
R₁₁ is 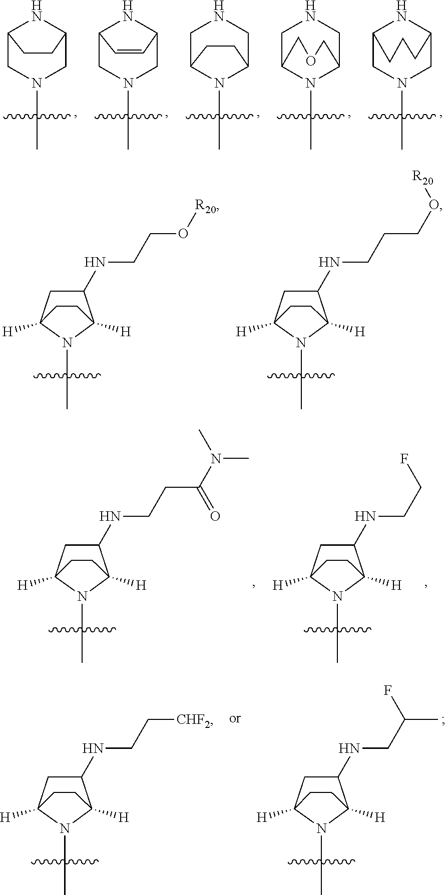
and
R₂₀ is methyl, —CHF₂, CF₃, —CH₂F, or —CD₃.

30. The bridged compound of claim 0,
wherein
R₄ is F, Cl or methyl;
R₆ₐ is H, F, Cl or methyl;
R_{6b} is F, Cl or methyl;
R_{6d} is H or F;
-L₁-R₈ is 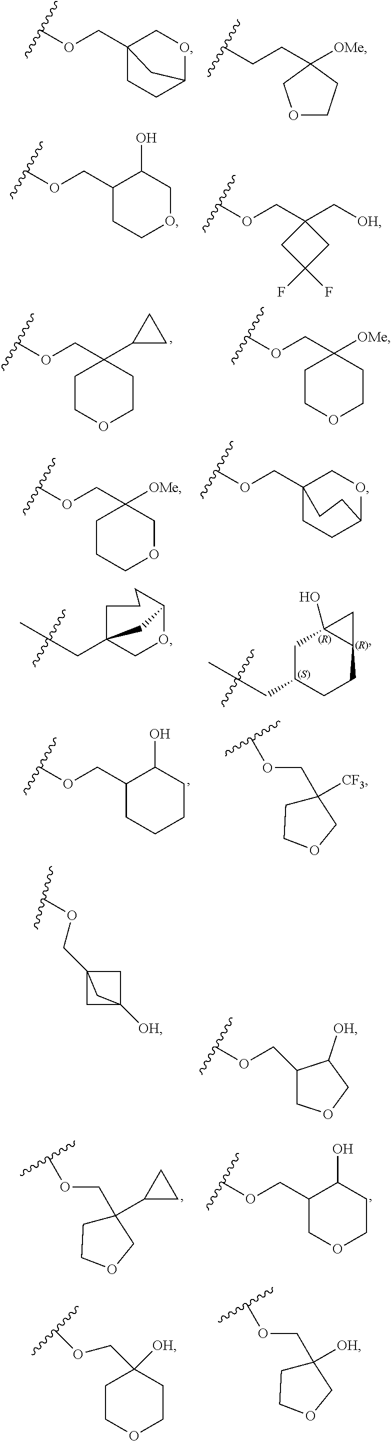 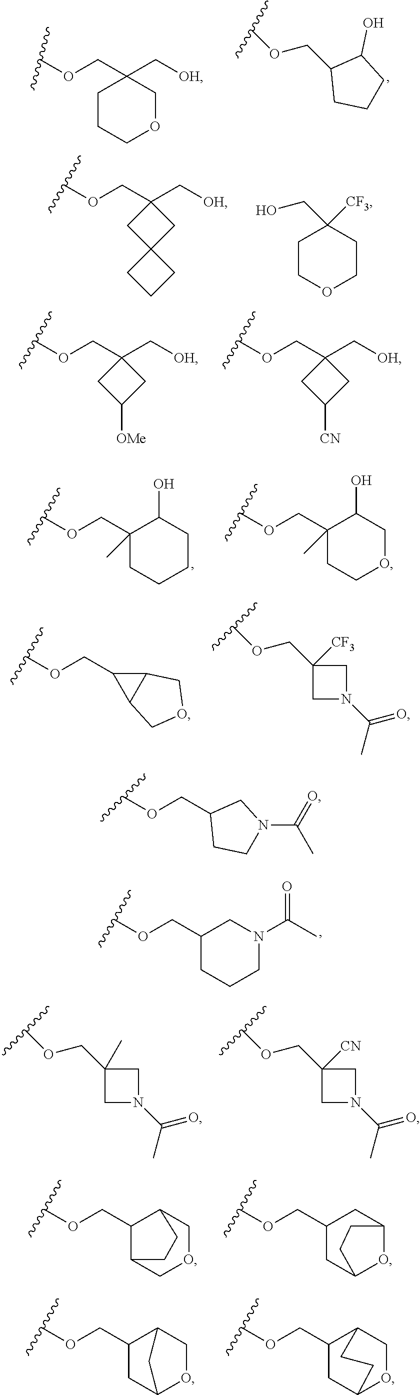 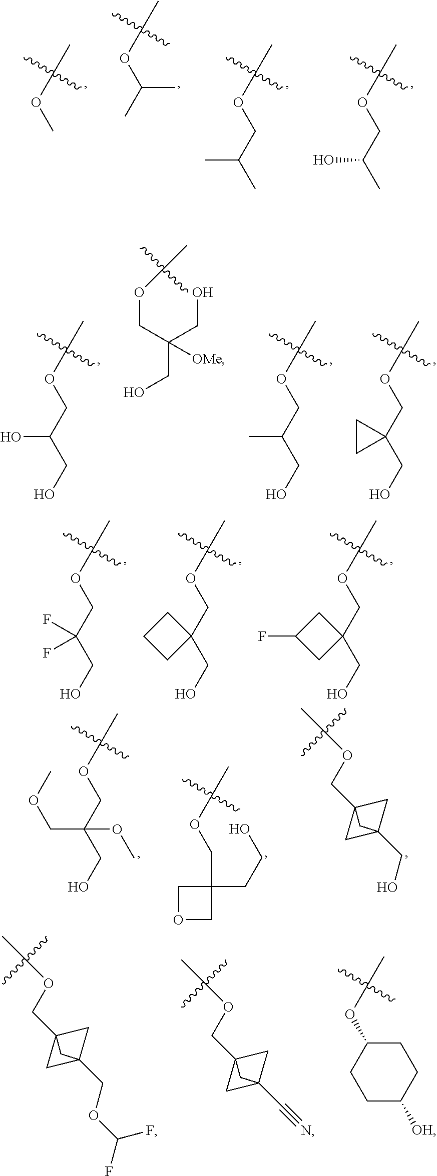 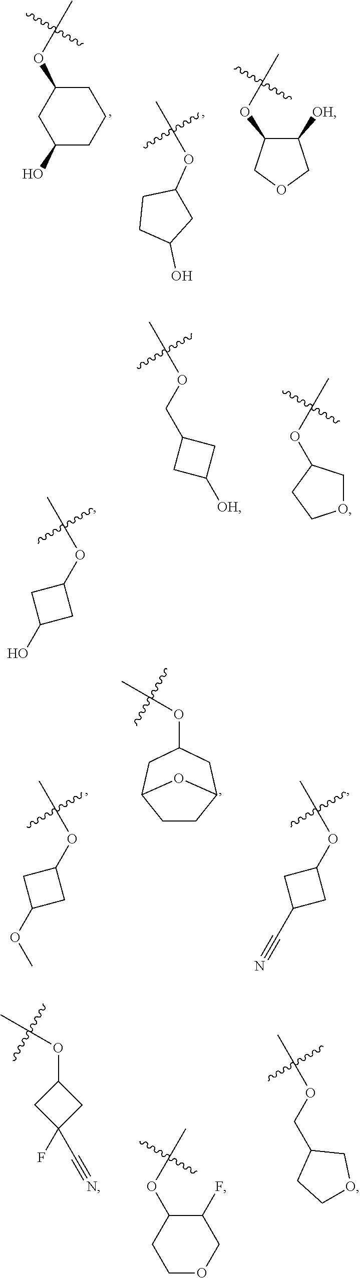 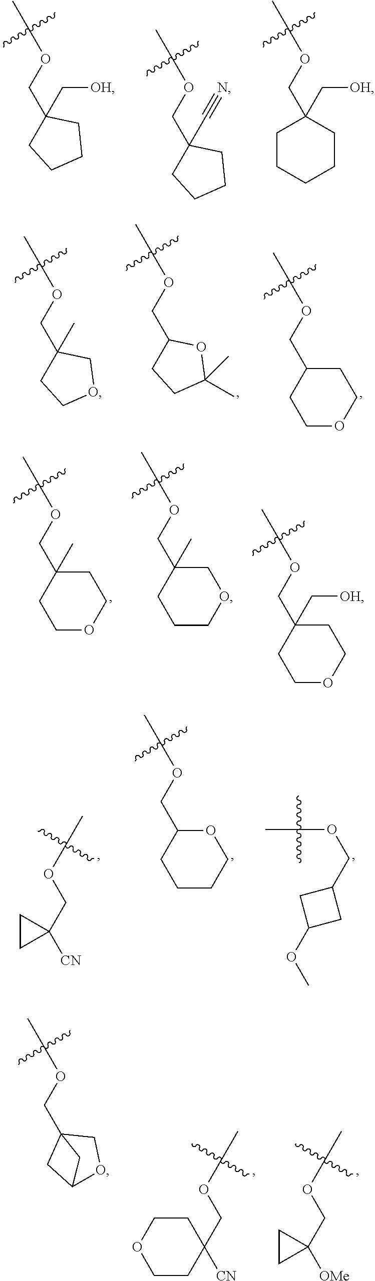 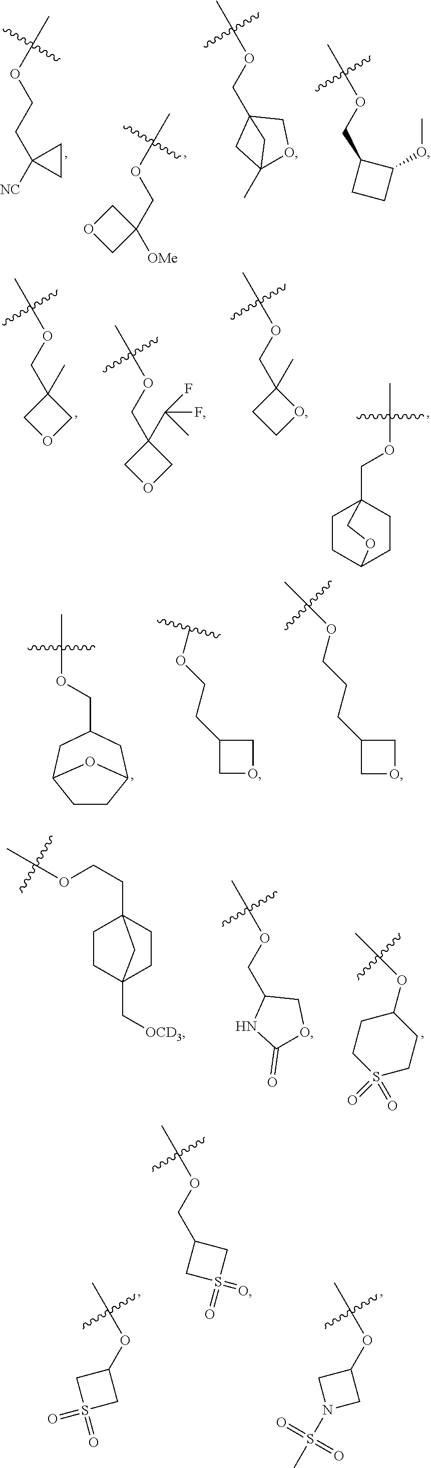 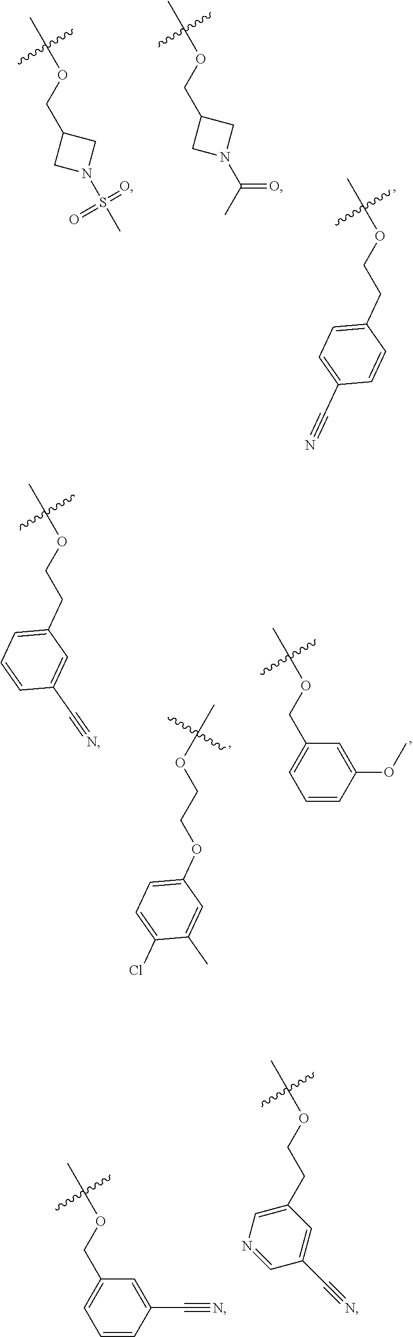 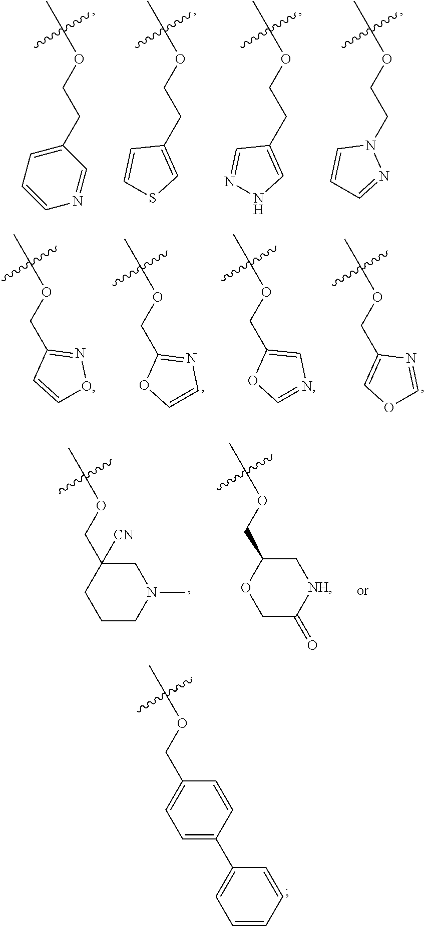
R₁₁ is 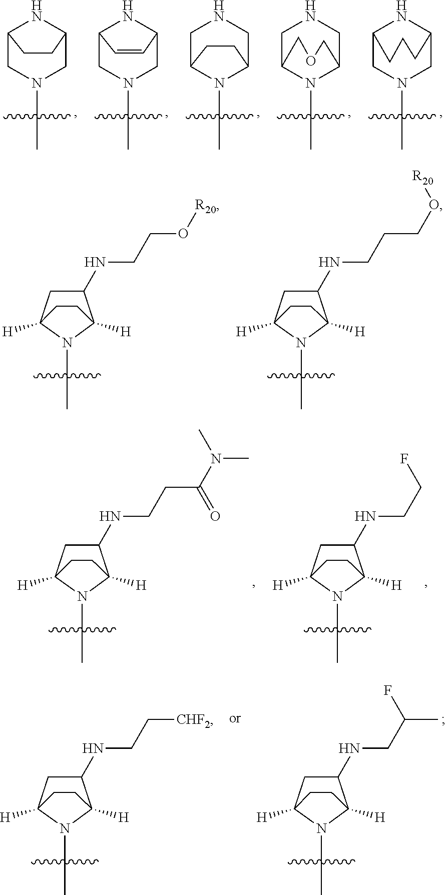
R₂₀ is methyl, —CHF₂, CF₃, —CH₂F, or —CD₃.

31. The bridged compound of claim 0,
wherein
R₄ is F;
R₅ is —CF₃;
R₆ₐ is H;
R_{6b} is F;
R_{6d} is H;
-L₁-R₈ is 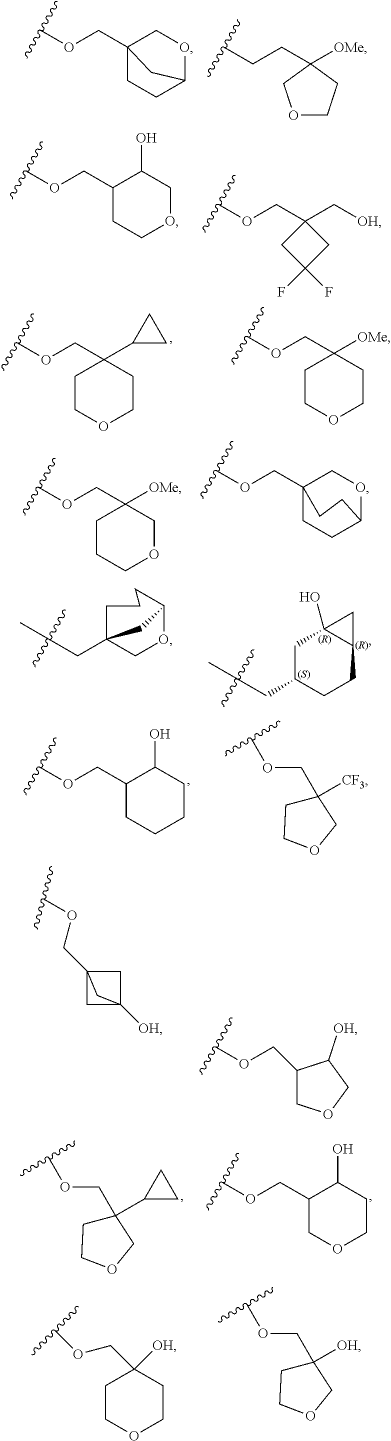 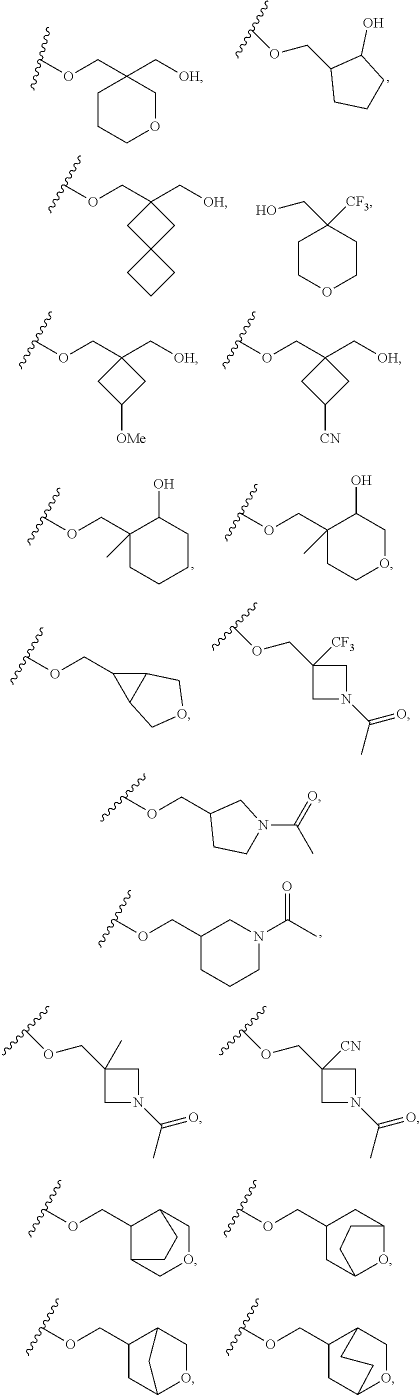 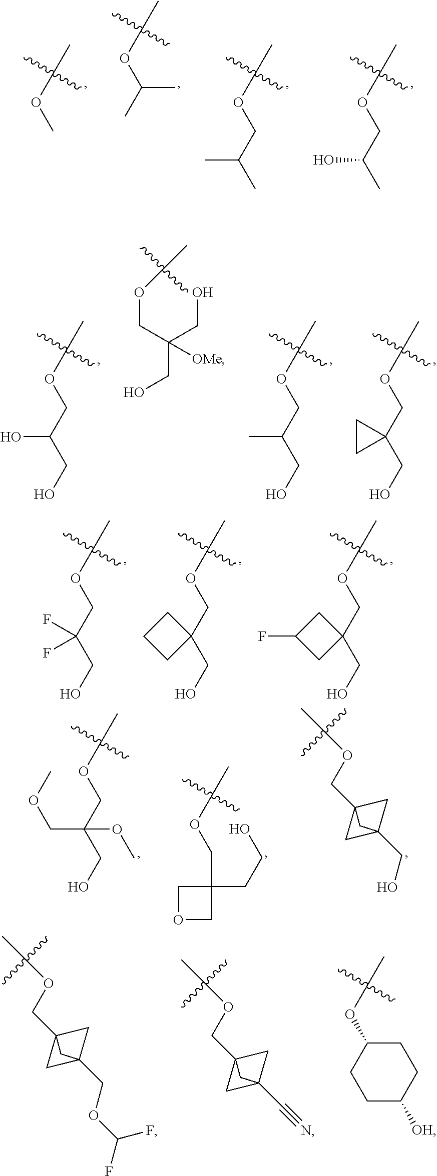 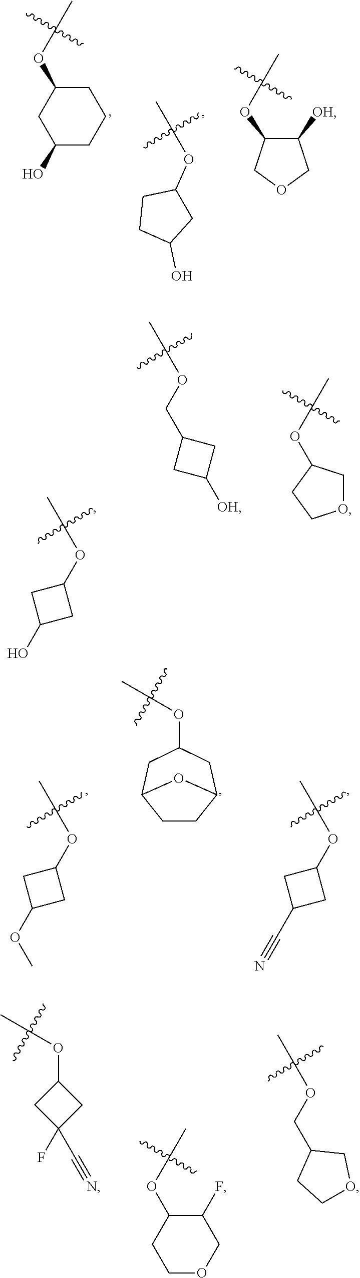 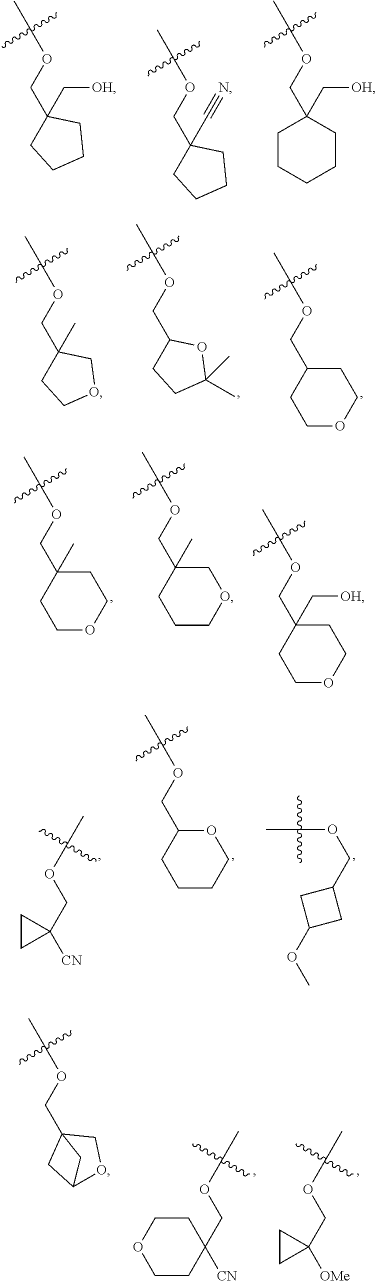 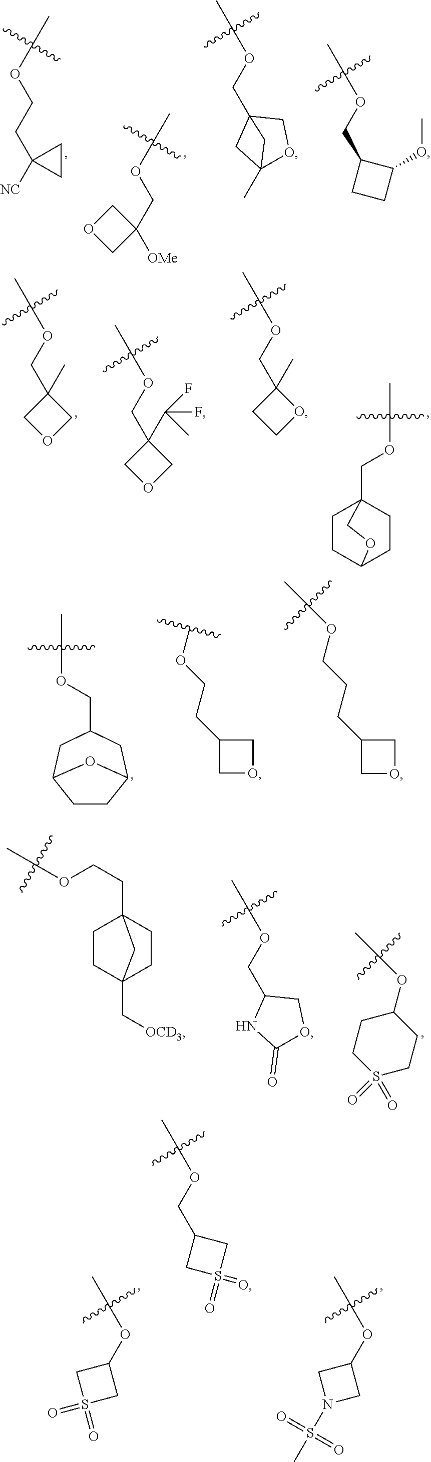 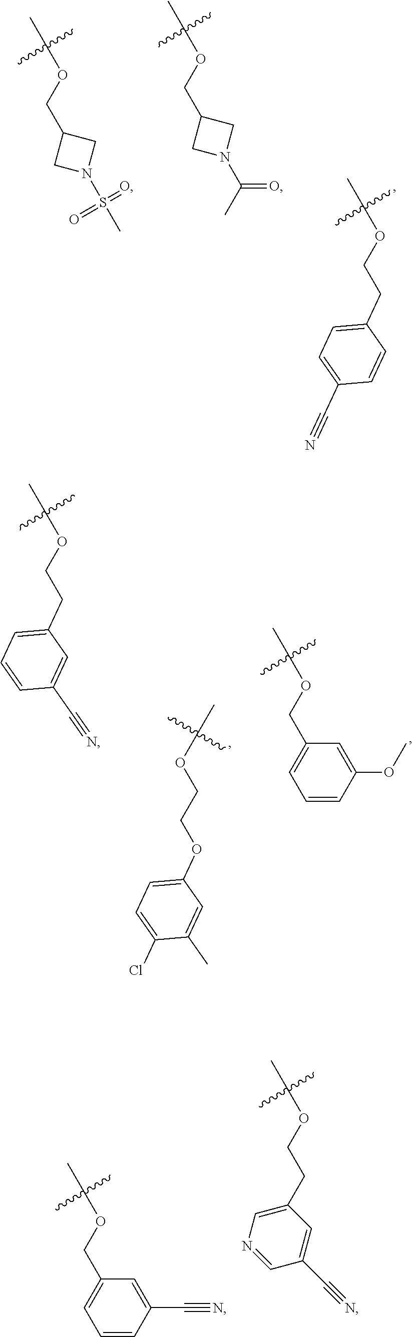 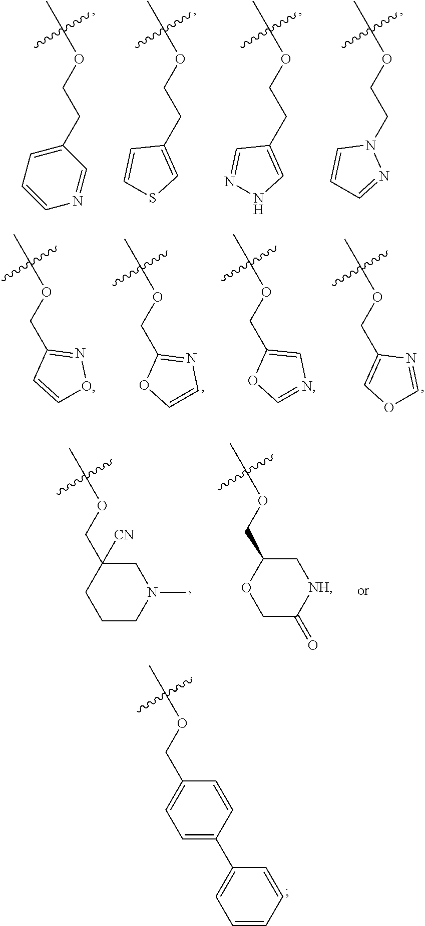
R₁₁ is

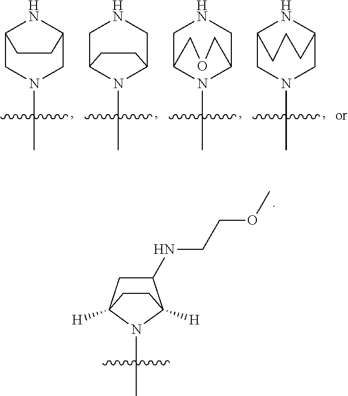

32. The bridged compound of claim 26, wherein A is C—CN.

33. A bridged compounds having formula (XIII): 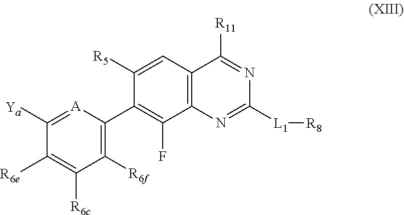
or a pharmaceutically acceptable salt, tautomer, or stereoisomer thereof,
wherein
A is N, CH or C—CN;
Yₐ is —NH₂ or —OH;
R₅ is —CF₃, —Cl, —OCF₃, —OCF₂Cl, —SCF₃, —H, —F, —Br, —I, —CF₂CH₃, —CH₂CHF₂, —CH₂CF₃, —CN, —CHF₂, ethyl, isopropyl, 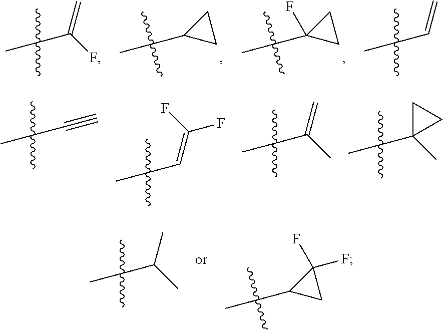
R_{6c} is H, F, Cl, methyl, ethyl, CHF₂, CN, Br, 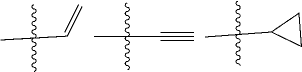
or CF₃;
R₆ₑ is H, F, or Cl;
R_{6f} is F, Cl, methyl, —CF₃, Br, I, —CF₂CH₃, —CH₂CF₃, —OCF₃, —OCF₂Cl, —SCF₃, isopropyl, cyclopropyl, or methylcyclopropyl;
-L₁- is —O—;
R₈ is unsubstituted or substituted alkyl, unsubstituted or substituted cycloalkyl, unsubstituted or substituted cycloalkylalkyl, unsubstituted or substituted aryl, unsubstituted or substituted aralkyl, unsubstituted or substituted heterocyclyl, unsubstituted or substituted heterocyclylalkyl, unsubstituted or substituted heteroaryl, unsubstituted or substituted heteroaralkyl, unsubstituted or substituted spirocyclic ring, or unsubstituted or substituted -alkyl-spirocyclic ring,
R₁₁ is

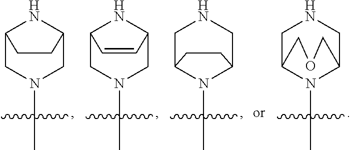

34. The bridged compound of claim 0, wherein the bridged compound is the compound having formula (XIV): 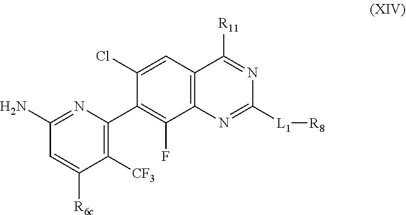
or a pharmaceutically acceptable salt, tautomer, or stereoisomer thereof,
wherein
R_{6c} is CH₃ or Cl;
-L₁- is —O—;
R₈ is unsubstituted or substituted alkyl, unsubstituted or substituted cycloalkyl, unsubstituted or substituted cycloalkylalkyl, unsubstituted or substituted aryl, unsubstituted or substituted aralkyl, unsubstituted or substituted heterocyclyl, unsubstituted or substituted heterocyclylalkyl, unsubstituted or substituted heteroaryl, unsubstituted or substituted heteroaralkyl, unsubstituted or substituted spirocyclic ring, or unsubstituted or substituted -alkyl-spirocyclic ring,
R₁₁ is

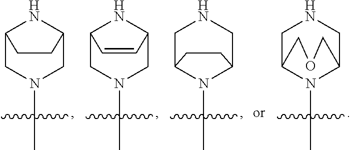

35. The bridged compound of claim 0,
wherein
R_{6c} is CH₃ or Cl;
-L₁-R₈ is 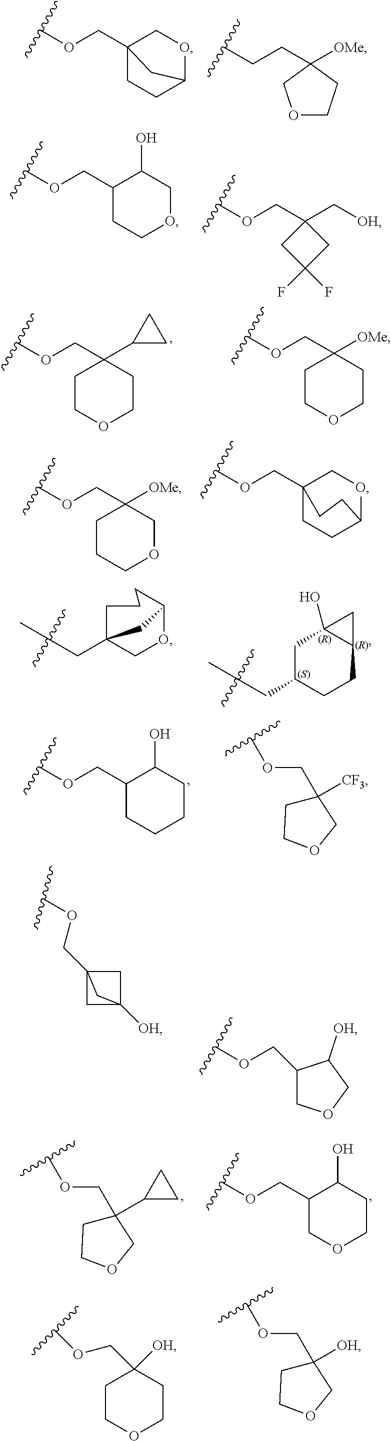 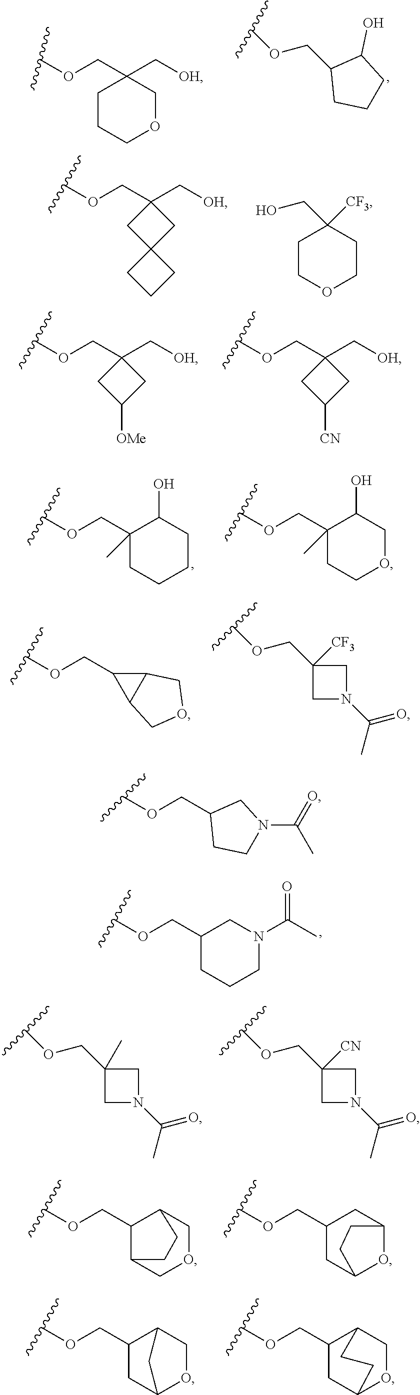 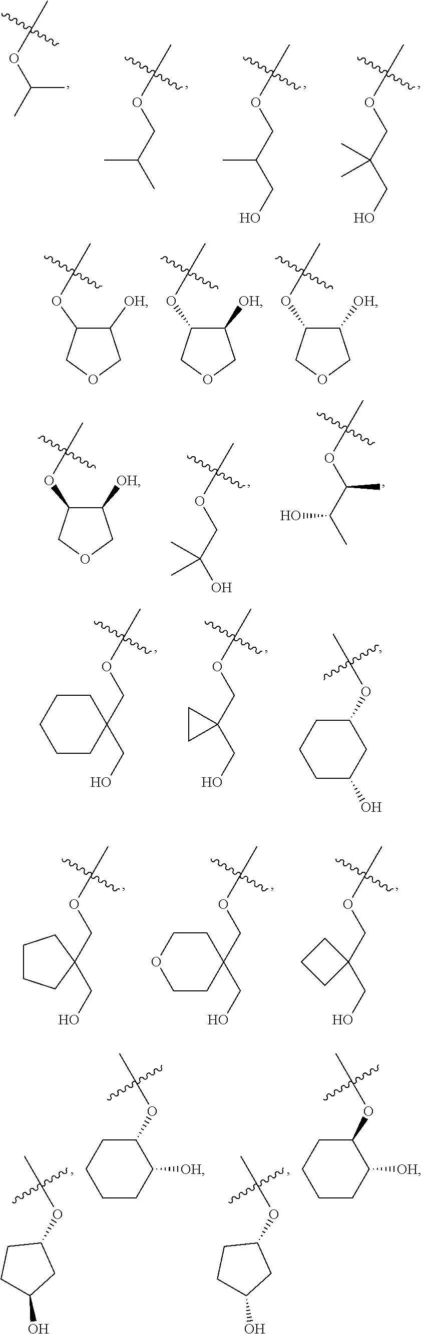 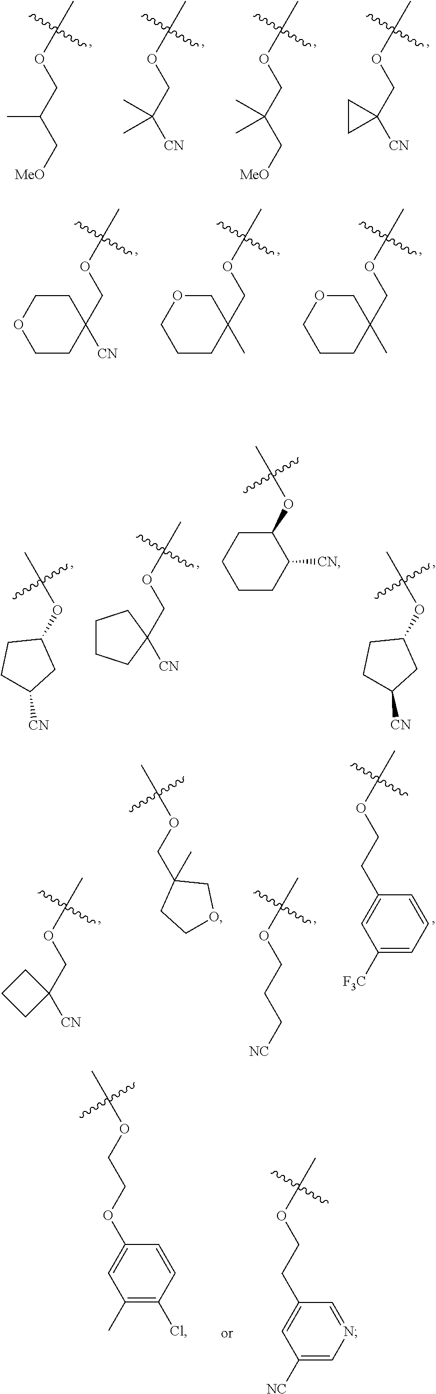
R₁₁ is

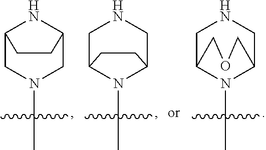

36. The bridged compound of claim 0,
wherein
R_{6c} is CH₃;
-L₁-R₈ is 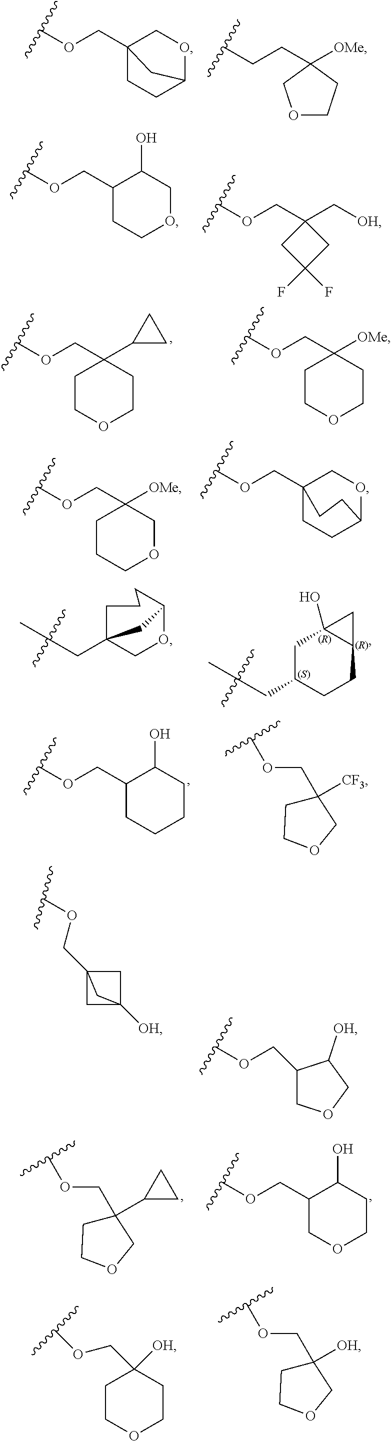 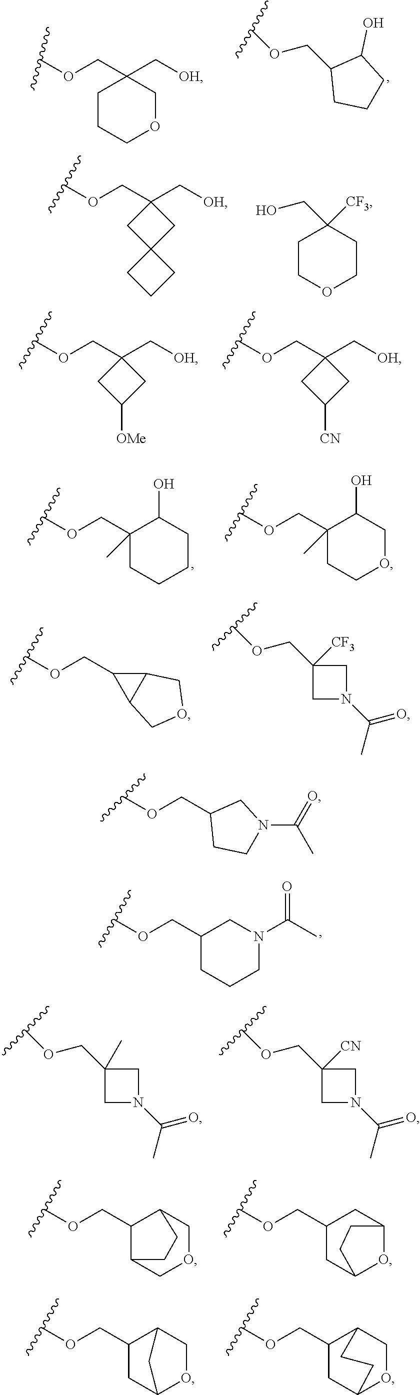 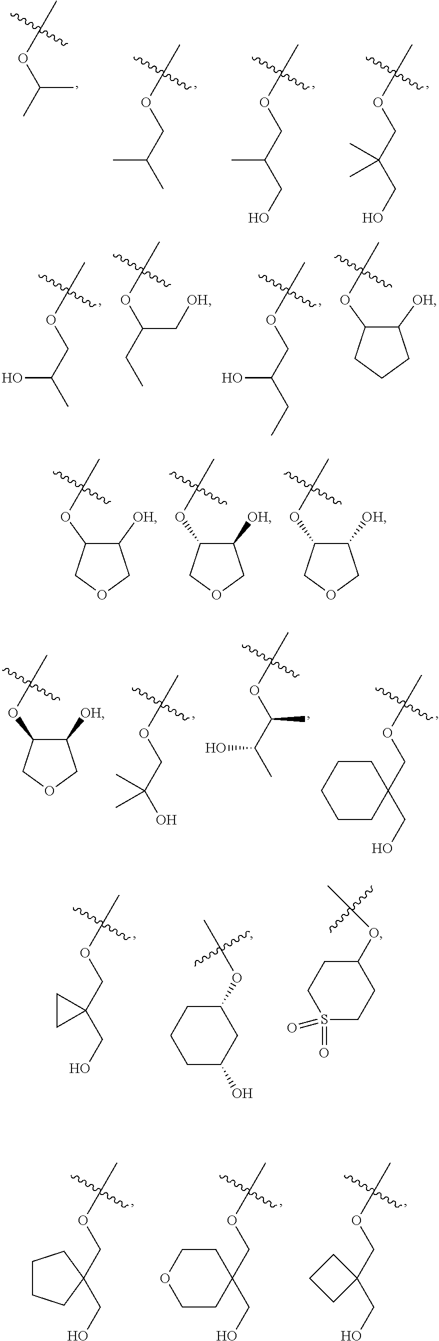 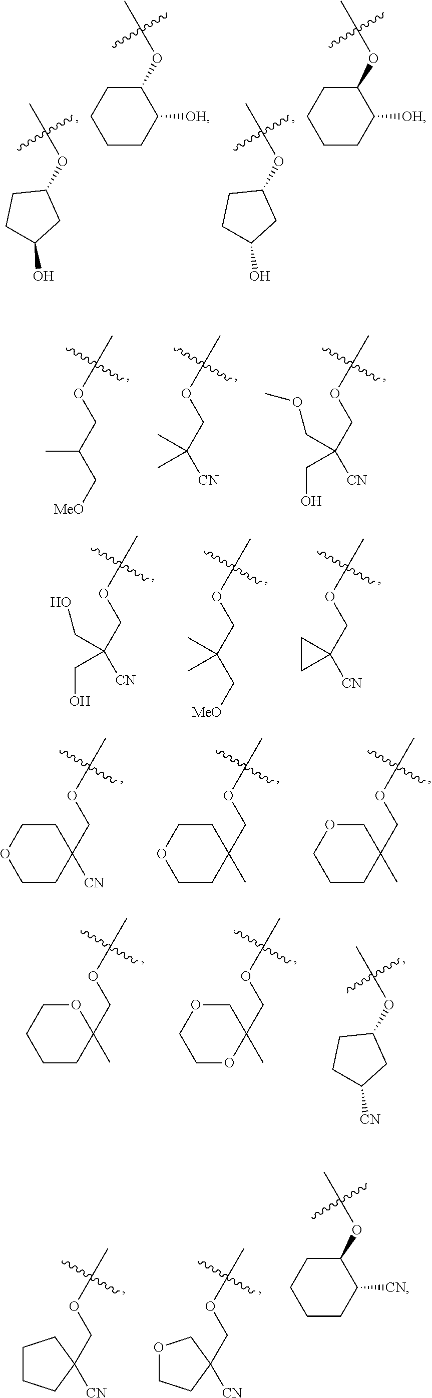 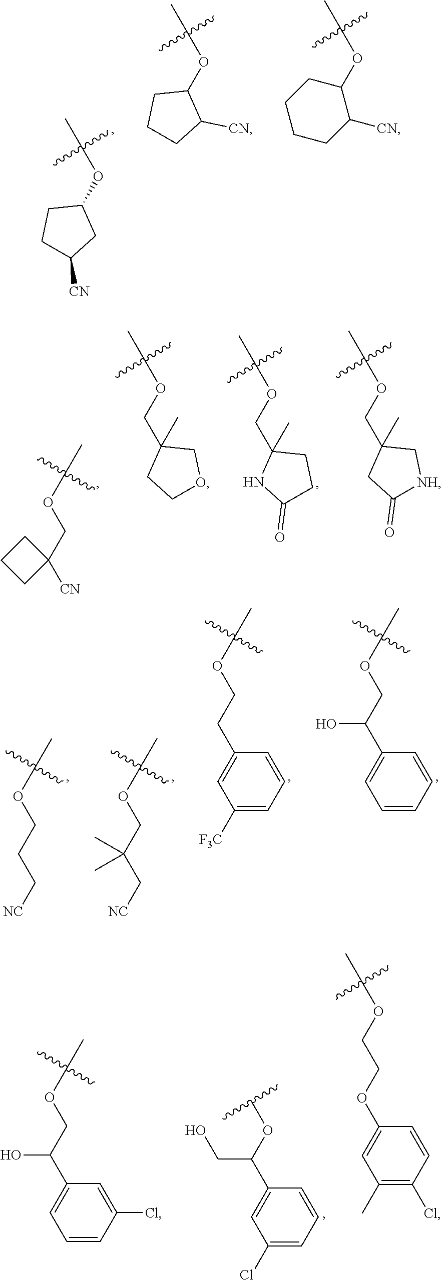 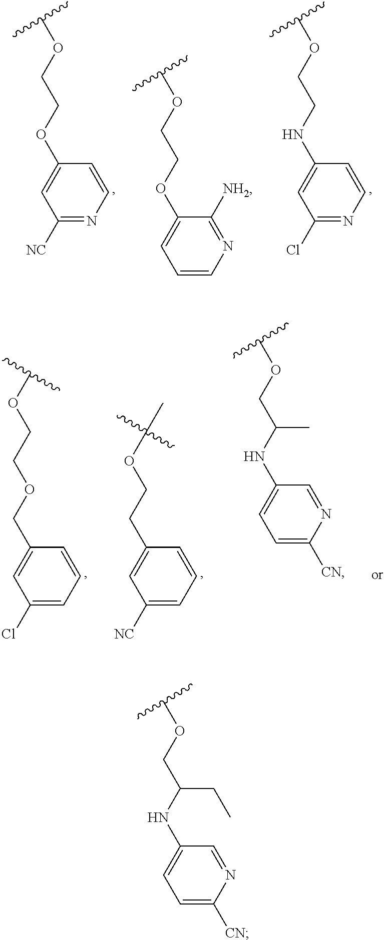
R₁₁ is

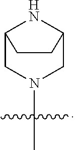 [SMILES: *N1CC2CCC(C1)N2]

37. The bridged compound of claim 0,
wherein
R_{6c} is CH₃;
-L₁-R₈ is 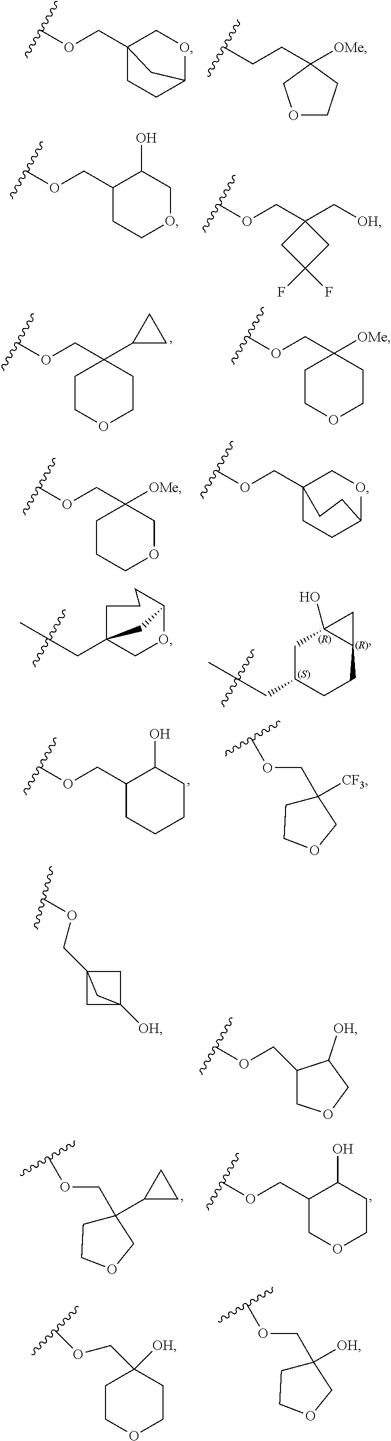 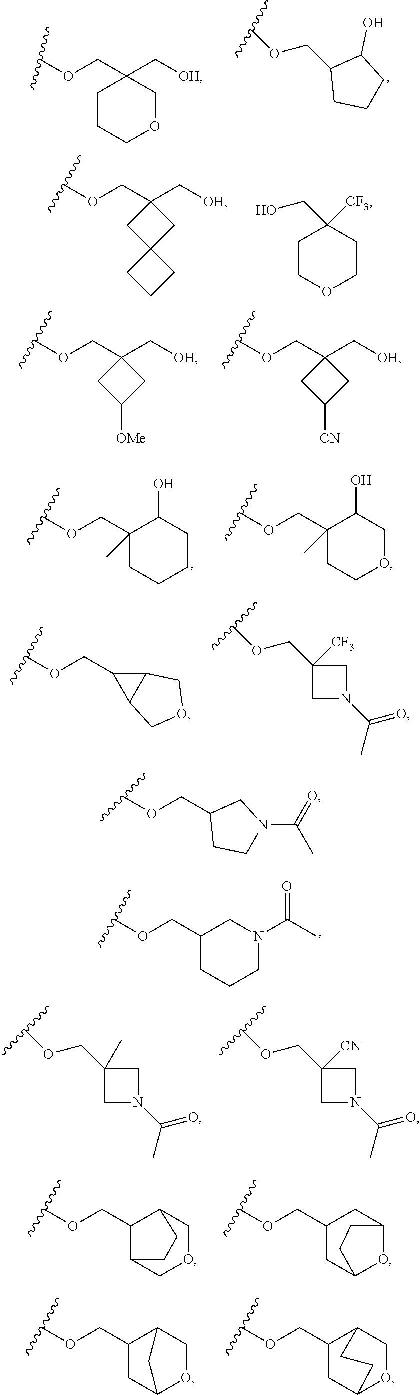 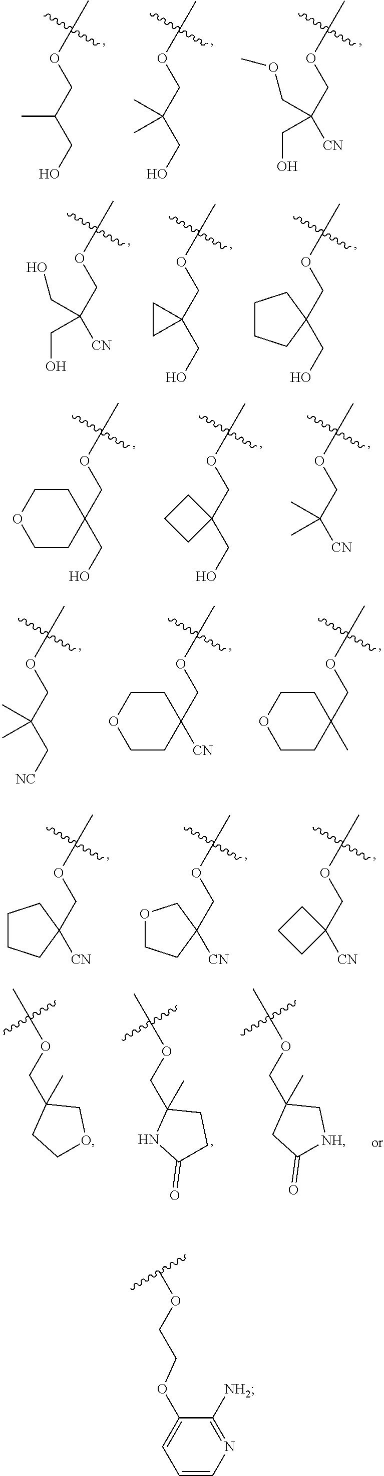
R₁₁ is

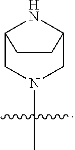 [SMILES: *N1CC2CCC(C1)N2]

38. The bridged compound of claim 1, where the bridged compound is selected from Error! Reference source not found, Error! Reference source not found, and Error! Reference source not found.

39.-42. (canceled)
